(19) United States
(12) Patent Application Publication
(10) Pub. No.: US 2024/0260455 A1
(43) Pub. Date: Aug. 1, 2024

(54) CHARGE TRANSPORT MATERIAL, COMPOUND, DELAYED FLUORESCENT MATERIAL AND ORGANIC LIGHT EMITTING ELEMENT

(71) Applicant: KYULUX, INC., Fukuoka-shi (JP)

(72) Inventors: YuSeok YANG, Fukuoka-shi (JP); Keiro NASU, Fukuoka-shi (JP); Asuka YOSHIZAKI, Fukuoka-shi (JP); Ping Kuen Daniel TSANG, Fukuoka-shi (JP); Ayataka ENDO, Fukuoka-shi (JP); Hiroko NOMURA, Fukuoka-shi (JP); Hidetoshi FUJIMURA, Fukuoka-shi (JP); Naoto NOTSUKA, Fukuoka-shi (JP)

(21) Appl. No.: 18/414,376

(22) Filed: Jan. 16, 2024

Related U.S. Application Data
(62) Division of application No. 16/304,532, filed on Nov. 26, 2018, now abandoned.

(30) Foreign Application Priority Data
Aug. 19, 2016 (JP) 2016-161561

(51) Int. Cl. H10K 85/60; C07D 405/14; C07D 409/14; C09K 11/06; H10K 50/00; H10K 50/11; H10K 101/10

(52) U.S. Cl. CPC H10K 85/654; C07D 405/14; C07D 409/14; C09K 11/06; H10K 50/00; H10K 85/6574; H10K 85/6576; C09K 2211/1018; H10K 50/11; H10K 2101/10

(57) ABSTRACT

A compound represented by the following general formula (1) is useful as a charge transport material and others in organic light emitting materials. Ar¹ to Ar³ each represents an aryl group or a heteroaryl group and at least one of Ar¹ to Ar³ contains a skeleton represented by the following general formula (2). In the general formula (2), X represents O or S. R¹ to R⁸ each represent a hydrogen atom, a substituent or a bonding position.

The entire disclosure of the prior parent application Ser. No. 16/304,532, filed Aug. 18, 2017, is considered as being part of the disclosure of the accompanying application and is hereby incorporated by reference therein.

### TECHNICAL FIELD

The present invention relates to a compound useful as a charge transport material and a delayed fluorescent material, and to an organic light emitting device using the compound.

### BACKGROUND ART

Studies for enhancing the light emission efficiency of organic light emitting devices such as organic electroluminescent devices (organic EL devices) are being made actively. In particular, various ingenious attempts to increase light emission efficiency by newly developing and combining electron transport materials, hole transport materials, light emitting materials and host materials to constitute organic electroluminescent devices have been made. Among them, studies relating to an organic electroluminescent device using a compound containing a 1,3,5-triazine structure are seen, and some proposals have heretofore been made.

For example, PTL 1 describes a technique of incorporating a compound that contains a 1,3,5-triazine structure represented by the following general formula, into the layer formed outside an electrode but not between two electrodes, to thereby improve optical efficiency. In the following general formula, Ar₂, Ar₄ and Ar₆ each represents a phenylene group or the like, b, d and f each represent an integer of 0 to 3, and R₂, R₄ and R₆ each are defined to be selected from a wide variety of groups such as a hydrogen atom, a halogen atom, an alkyl group, an aryl group and the like. However, the patent literature does not describe a group containing a dibenzofuran structure or a dibenzothiophene structure as R₂, R₄ and R₆.

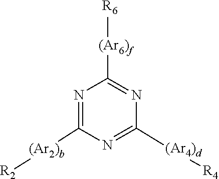

### CITATION LIST

### Patent Literature

PTL 1: JP 2010-45034 A

### SUMMARY OF INVENTION

### Technical Problem

As in the above, some investigations have heretofore been made relating to compounds containing a 1,3,5-triazine structure. However, few concrete investigations have been made relating to a compound containing a 1,3,5-triazine structure, and a dibenzofuran skeleton or a dibenzothiophene skeleton in the molecule thereof. In particular, reports disclosing examples of a compound containing both a 1,3,5-triazine structure where the 2-position, the 4-position and the 6-position are substituted with an aryl group or a heteroaryl group, and a dibenzofuran skeleton or a dibenzothiophene skeleton are limited. Consequently, it is extremely difficult to accurately anticipate as to what properties the compound having these structures as combined therein could exhibit. In particular, regarding the usefulness of such a compound as a host material in a light emitting layer, it is absolutely difficult to find out any literature that could be a ground for anticipation, as obvious from the fact that PTL 1 does not describe at all the use as a host material.

The present inventors have taken the problems in the related art into consideration, and have promoted investigations of synthesizing a compound having both a 1,3,5-triazine structure, and a dibenzofuran skeleton or a dibenzothiophene skeleton in the molecule thereof and evaluating the usefulness the compound as a material for organic light emitting devices. In addition, the inventors have further promoted assiduous studies for the purpose of deriving a general formula of a compound useful as a material for organic light emitting devices and generalizing the structure of an organic light emitting device having a high light emission efficiency.

### Solution to Problem

As a result of assiduous studies made for the purpose of attaining the above-mentioned object, the present inventors have succeeded in synthesizing compounds having a 1,3,5-triazine structure where the 2-position, the 4-position and the 6-position are substituted with an aryl group or a heteroaryl group, and a dibenzofuran skeleton or a dibenzothiophene skeleton, and have clarified for the first time that the compounds are useful as a material for organic light emitting devices. Based on these findings, the present inventors have provided the present invention described hereinunder, as a means for solving the above-mentioned problems.
[1] A charge transport material containing a compound represented by the following general formula (1):

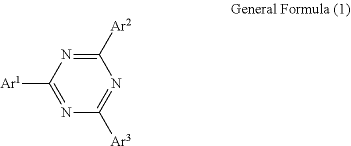

wherein Ar¹ to Ar³ each independently represent a substituted or unsubstituted aryl group or a substituted or unsubstituted heteroaryl group, and at least one of Ar¹ to Ar³ contains a skeleton represented by the following formula (2), but Ar¹ to Ar³ do not contain a 4-(benzofuran-1-yl)carbazol-9-yl group or a 4-(benzothiophen-1-yl)carbazol-9-yl group,

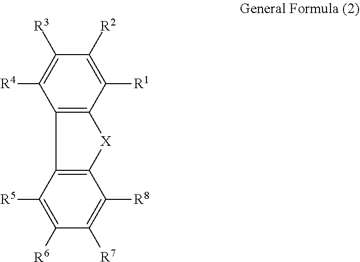

wherein X represents O or S, R¹ to R⁸ each independently represent a hydrogen atom, a substituent or a bonding position, R¹ and R², R² and R³, R³ and R⁴, R⁵ and R⁶, R⁶ and R⁷, and R⁷ and R⁸ each may bond to each other to form a cyclic structure.
[2] The charge transport material according to [1], wherein 2 or more skeletons represented by the general formula (2) exist in the molecule.
[3] The charge transport material according to [1] or [2], wherein two of Ar¹ to Ar³ in the general formula (1) contain the skeleton represented by the general formula (2).
[4] The charge transport material according to [1] or [2], wherein one of Ar¹ to Ar³ in the general formula (1) contains the skeleton represented by the general formula (2).
[5] The charge transport material according to any one of [1] to [4], wherein one of Ar¹ to Ar³ in the general formula (1) contains 2 or more skeletons represented by the general formula (2).
[6] The charge transport material according to any one of [1] to [5], wherein the group containing the skeleton represented by the general formula (2) is a group bonding at the bonding position of R¹ in the general formula (2).
[7] The charge transport material according to any one of [1] to [5], wherein the group containing the skeleton represented by the general formula (2) is a group bonding at the bonding position of R⁴ in the general formula (2).
[8] The charge transport material according to any one of [1] to [7], wherein at least one of Ar¹ to Ar³ in the general formula (1) is an aryl group substituted with a group containing the skeleton represented by the general formula (2), or a heteroaryl group substituted with a group containing the skeleton represented by the general formula (2).
[9] The charge transport material according to [8], wherein the aryl group substituted with a group containing the skeleton represented by the general formula (2) has such a structure that the skeleton represented by the general formula (2) bonds to the aryl group at the bonding position of any one of R¹ to R⁸ via a single bond therebetween.
[10] The charge transport material according to [9], wherein the skeleton represented by the general formula (2) bonds to the aryl group at the bonding position of R¹ or R⁴ via a single bond therebetween.
[11] The charge transport material according to [9] or [10], wherein the aryl group is a phenyl group, and the skeleton represented by the general formula (2) bonds to both the meta-positions of the phenyl group relative to the bonding position to the triazine ring, each via a single bond therebetween.
[12] The charge transport material according to [9] or [10], wherein the aryl group is a phenyl group, and the skeleton represented by the general formula (2) bonds to the para-position of the phenyl group relative to the bonding position to the triazine ring via a single bond therebetween.
[13] The charge transport material according to [8], wherein the heteroaryl group substituted with a group containing the skeleton represented by the general formula (2) has such a structure that the skeleton represented by the general formula (2) bonds to the heteroaryl group at the bonding position of any one of R¹ to R⁸ via a single bond therebetween.
[14] The charge transport material according to [8], wherein the heteroaryl group substituted with a group containing the skeleton represented by the general formula (2) contains a carbazole ring, and the skeleton represented by the general formula (2) bonds to the carbazole ring at the bonding position of any one of R¹ to R⁸ via a single bond therebetween.
[15] The charge transport material according to [14], wherein the group containing the skeleton represented by the formula (2) is a group represented by the following general formula (3):

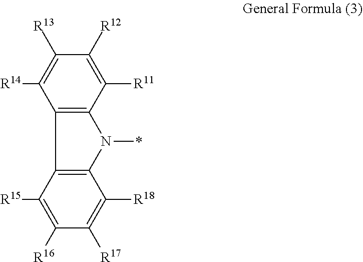

wherein * represents a bonding position, R¹¹ to R¹⁸ each independently represent a hydrogen atom or a substituent, at least one of R¹¹ to R¹⁸ is a skeleton represented by the general formula (2) and bonding to the carbazole ring at the bonding position of any one of R¹ to R⁸ via a single bond therebetween, R¹¹ and R¹², R¹² and R¹³, R¹³ and R¹⁴, R¹⁵ and R¹⁶, R¹⁶ and R¹⁷, and R¹⁷ and R¹⁸ each may bond to each other to form a cyclic structure.
[16] The charge transport material according to [15], wherein at least one of R¹³ and R¹⁶ in the general formula (3) is a skeleton represented by the general formula (2) and bonding to the carbazole ring at the bonding position of any one of R¹ to R⁸ via a single bond therebetween.
[17] The charge transport material according to [15] or [16], wherein the skeleton represented by the general formula (2) bonds to the carbazole ring of the general formula (3) at the bonding position of R¹ via a single bond therebetween.
[18] The charge transport material according to any one of [1] to [17], wherein in the group containing the skeleton represented by the general formula (2), at least one combination of R¹ and R², R² and R³, R³ and R⁴, R⁶ and R⁶, R⁶ and R⁷, and R⁷ and R⁸ bonds to each other to form an indole ring.
[19] The charge transport material according to [18], wherein the group containing the skeleton represented by the general formula (2) is a group represented by any of the following formulae (where * indicates a bonding position):

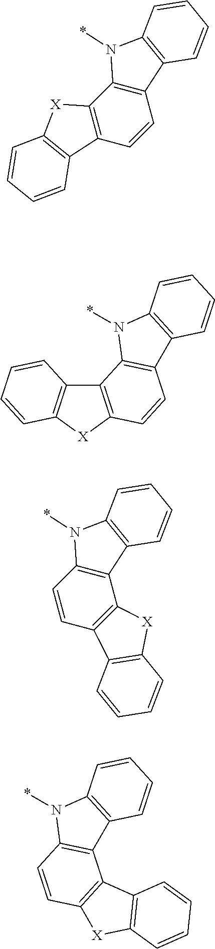

wherein X represents O or S, * represents a bonding position, and the methine group in the formulae may be substituted with a substituent.
[20] The charge transport material according to any one of [8] to [19], wherein the aryl group substituted with a group containing the skeleton represented by the general formula (2) or the heteroaryl group substituted with a group containing the skeleton represented by the general formula (2) is further substituted with an alkyl group.
[21] The charge transport material according to any one of [1] to [20], wherein the compound represented by the general formula (1) is a compound represented by the following general formula (4):

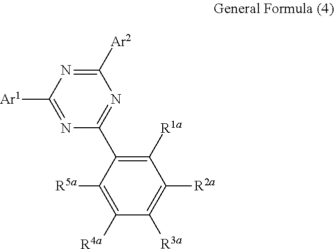

wherein Ar¹ and Ar² each independently represent a substituted or unsubstituted aryl group or a substituted or unsubstituted heteroaryl group, R^{1a} to R^{5a} each independently represent a hydrogen atom or a substituent, at least one of R^{1a}, R^{3a} and R^{5a} contains a skeleton represented by the general formula (2), however, Ar¹, Ar² and R^{1a} to R^{5a} do not contain a 4-(benzofuran-1-yl)carbazole-9-yl group or a 4-(benzothiophen-1-yl)carbazol-9-yl group, and R^{1a} and R^{2a}, R^{2a} and R^{3a}, R^{3a} and R^{4a}, and R^{4a} and R^{5a} each may independently bond to each other to form a cyclic structure.
[22] The charge transport material according to [21], wherein in the general formula (4), R^{3a} contains a skeleton represented by the general formula (2).
[23] The charge transport material according to [22], wherein in the general formula (4), R^{3a} contains a skeleton represented by the general formula (2), and R^{1a}, R^{2a}, R^{4a} and R^{5a} do not contain a skeleton represented by the general formula (2).
[24] The charge transport material according to any one of [21] to [23], wherein in the general formula (4), Ar² contains a skeleton represented by the general formula (2).
[25] The charge transport material according to any one of [1] to [20], wherein the compound represented by the general formula (1) is a compound represented by the following general formula (5):

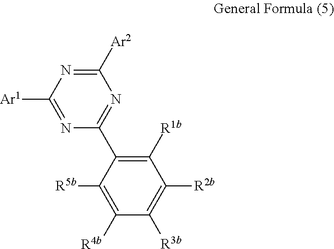

wherein Ar¹ and Ar² each independently represent a substituted or unsubstituted aryl group or a substituted or unsubstituted heteroaryl group, R^{1b} to R^{5b} each independently represent a hydrogen atom or a substituent, and at least one of Rib, R^{3b}, R^{4b} and R^{5b}, and R^{2b} each independently contain a skeleton represented by the general formula (2), but Ar¹, Ar² and R^{1b} to R^{5b} do not contain a 4-(benzofuran-1-yl)carbazol-9-yl group or a 4-(benzothiophen-1-yl)carbazol-9-yl group, and R^{1b} and R^{2b}, R^{2b} and R^{3b}, R^{3b} and R^{4b}, and R^{4b} and R^{5b} each may independently bond to each other to form a cyclic structure.
[26] The charge transport material according to [25], wherein in the general formula (5), R^{4b} contains a skeleton represented by the general formula (2).
[27] The charge transport material according to any one of [1] to [20], wherein the compound represented by the general formula (1) is a compound represented by the following general formula (6):

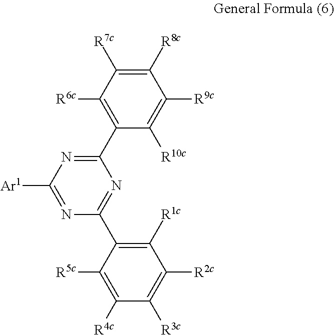

wherein Ar¹ represents a substituted or unsubstituted aryl group or a substituted or unsubstituted heteroaryl group, R^{1c} to R^{10c} each independently represent a hydrogen atom or a substituent, but at least one of R^{6c} to R^{10c}, and R^{2c} each independently contains a skeleton represented by the general formula (2), however, R^{7c} in the case where only R^{2c} and R^{7c} among R^{1c} to R^{10c} contain a skeleton represented by the general formula (2) is not the same as R^{2c}, and in the case where a dibenzofuran ring exists in R^{2c}, the group is not a group where the oxygen atom in the dibenzofuran ring is substituted with a sulfur atom, and in the case where a dibenzothiophene ring exists in R^{2c}, the group is not a group where the sulfur atom in the dibenzothiophene ring is substituted with an oxygen atom, Ar¹, Ar² and R^{1c} to R^{10c} do not contain a 4-(benzofuran-1-yl)carbazol-9-yl group or a 4-(benzothiophen-1-yl)carbazol-9-yl group, and R^{1c} and R^{2c}, R^{2c} and R^{3c}, R^{3c} and R^{4c}, R^{4c} and R^{5c}, R^{6c} and R^{7c}, R^{7c} and R^{8c}, R^{8c} and R^{9c}, and R^{9c} and R^{10c} each may independently bond to each other to form a cyclic structure.
[28] The charge transport material according to [27], wherein in the general formula (6), at least two of R^{1c} to R^{5c}, and at least two of R^{6c} to R^{10c} each independently contain a skeleton represented by the general formula (2).
[29] The charge transport material according to [27] or [28], wherein in the general formula (6), R^{2c} is a group containing a dibenzofuran-x-yl group or a dibenzothiophen-x-yl group, at least one of R^{6b} to R^{10b} is a group containing a dibenzofuran-y-yl group or a dibenzothiophen-y-yl group, x and y each are a number indicating the bonding position of the dibenzofuryl group or the dibenzothienyl group, and x and y are not the same.
[30] The charge transport material according to any one of [1] to [29], which is used in combination with a delayed fluorescent material.
[31] The charge transport material according to [30], which is a host material to be used in combination with a delayed fluorescent material.
[32] The charge transport material according to [30], which is a hole blocking material to be used in combination with a delayed fluorescent material.
[33] The charge transport material according to [30], which is an electron transport material to be used in combination with a delayed fluorescent material.
[34] A compound represented by the above-mentioned general formula (1).
[35] The compound according to [34], wherein, when only one of Ar¹ to Ar³ in the general formula (1) is a phenyl group substituted with only one group containing a skeleton represented by the general formula (2), and the group containing a skeleton represented by the general formula (2) is a group represented by the following general formula (A), and among R^{12a} to R^{16a}, only one of R^{12a} to R^{14a} is a skeleton represented by the general formula (2),
   the phenyl group substituted with only one group containing a skeleton represented by the general formula (2) is further substituted with an alkyl group, or at least one of R^{11a} to R^{18a} is an alkyl group, or excepting for the case where the phenyl group substituted with only one group containing a skeleton represented by the general formula (2) is further substituted with an alkyl group, and where at least one of R^{11a} to R^{18a} is an alkyl group, the skeleton represented by the general formula (2) bonds to the carbazole ring in the general formula (A) at the bonding position of R² or R³ via a single bond therebetween:

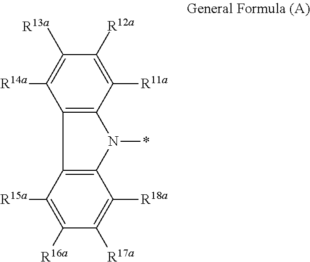

wherein * represents a bonding position, R^{11a} to R^{18a} each independently represent a hydrogen atom or a substituent, one or two of R^{12a} to R^{16a} is a skeleton represented by the general formula (2) and bonding to the carbazole ring at the bonding position of any one of R¹ to R⁸ via a single bond therebetween, but among R^{12a} to R^{16a}, only one of R₁₂ₐ to R₁₄ₐ or both R^{13a} and R^{16a} alone is/are a skeleton represented by the general formula (2), and R^{11a} and R^{12a}, R^{12a} and R¹³, R^{13a} and R₁₄ₐ, R^{15a} and R^{16a}, R^{16a} and R^{17a}, and R^{17a} and R^{18a} each may bond to each other to form a cyclic structure.
[36] The compound according to [34] or [35], wherein, when only one of Ar¹ to Ar³ in the general formula (1) is a phenyl group substituted with only one group containing a skeleton represented by the general formula (2), and when the group containing the skeleton represented by the general formula (2) is a group represented by the general formula (A), and among R^{12a} to R^{16a}, R^{13a} and R^{16a} alone are a skeleton represented by the general formula (2),
   the substituting position of the group containing the skeleton represented by the general formula (A) to the phenyl group is an ortho-position or a para-position relative to the bonding position of the triazine ring in the general formula (1).
[37] The compound according to any one of [34] to [36], wherein, when only two of Ar¹ to Ar³ in the general formula (1) are an aryl group substituted with a group containing a skeleton represented by the general formula (2), and when the aryl group is a phenyl group to which only one skeleton represented by the general formula (2) bonds at the bonding position R¹ via a single bond therebetween,
   R⁶ in the general formula (2) is not a pyrimidinyl group, and the bonding position to the phenyl group of the skeleton represented by the general formula (2) is an ortho-position or a meta-position relative to the bonding position of the triazine ring in the general formula (1).
[38] The compound according to [34], wherein the compound represented by the general formula (1) is a compound represented by the general formula (4).
[39] The compound according to [34], wherein the compound represented by the general formula (1) is a compound represented by the general formula (5).
[40] The compound according to [34], wherein the compound represented by the general formula (1) is a compound represented by the general formula (6).
[41] A delayed fluorescent material containing a compound according to any one of [34] to [40].
[42] An organic light emitting device containing a compound represented by the general formula (1).
[43] The organic light emitting device according to [42], which radiates delayed fluorescence.
[44] The organic light emitting device according to [42] or [43], which contains a compound represented by the general formula (1) and a delayed fluorescent material in the light emitting layer therein.
[45] The organic light emitting device according to [44], wherein the content of the compound in the light emitting layer is more than 50% by weight.
[46] The organic light emitting device according to [42] or [43], containing the compound represented by the general formula (1) in a layer adjacent to the light emitting layer.

### Advantageous Effects of Invention

The compounds of the present invention have high heat stability and are useful as materials for organic light emitting devices. Above all, the compounds of the present invention include compounds useful as host materials, hole blocking materials, electron transport materials and delayed fluorescent materials for organic light emitting devices. An organic light emitting material using such a compound of the present invention as a host material or a delayed fluorescent material for the light emitting layer, or as a material for the hole blocking layer or the electron transport layer therein can realize high light emission efficiency and high heat stability.

### BRIEF DESCRIPTION OF DRAWINGS

FIG. 1 This is a schematic cross-sectional view showing a layer configuration example of an organic electroluminescent device.

FIGS. 2A and 2B This includes graphs each showing device characteristics of the organic electroluminescent device produced in Example 2, as measured before and after heating the device at 80° C. for 12 hours, in which (FIG. 2A) is a graph showing the voltage-current density characteristics of the device, and (FIG. 2B) is a graph showing the current density-external quantum efficiency characteristics of the device.

FIGS. 3A and 3B This includes graphs each showing device characteristics of the organic electroluminescent device produced in Example 3, as measured before and after heating the device at 80° C. for 12 hours, in which (FIG. 3A) is a graph showing the voltage-current density characteristics of the device, and (FIG. 3B) is a graph showing the current density-external quantum efficiency characteristics of the device.

FIGS. 4A and 4B This includes graphs each showing device characteristics of the organic electroluminescent device produced in Example 4, as measured before and after heating the device at 80° C. for 12 hours, in which (FIG. 4A) is a graph showing the voltage-current density characteristics of the device, and (FIG. 4B) is a graph showing the current density-external quantum efficiency characteristics of the device.

FIGS. 5B and 5C This includes graphs each showing device characteristics of the organic electroluminescent device produced in Example 5, as measured before and after heating the device at 80° C. for 12 hours, in which (FIG. 5B) is a graph showing the voltage-current density characteristics of the device, and (FIG. 5C) is a graph showing the current density-external quantum efficiency characteristics of the device.

FIGS. 6A and 6B This includes graphs each showing device characteristics of the organic electroluminescent device produced in Example 6, as measured before and after heating the device at 80° C. for 12 hours, in which (FIG. 6A) is a graph showing the voltage-current density characteristics of the device, and (FIG. 6B) is a graph showing the current density-external quantum efficiency characteristics of the device.

FIGS. 7A and 7B This includes graphs each showing device characteristics of the organic electroluminescent device produced in Example 7, as measured before and after heating the device at 80° C. for 12 hours, in which (FIG. 7A) is a graph showing the voltage-current density characteristics of the device, and (FIG. 7B) is a graph showing the current density-external quantum efficiency characteristics of the device.

FIGS. 8A and 8B This includes graphs each showing device characteristics of the organic electroluminescent device produced in Example 8, as measured before and after heating the device at 80° C. for 12 hours, in which (FIG. 8A) is a graph showing the voltage-current density characteristics of the device, and (FIG. 8B) is a graph showing the current density-external quantum efficiency characteristics of the device.

FIGS. 9A and 9B This includes graphs each showing device characteristics of the organic electroluminescent device produced in Example 9, as measured before and after heating the device at 80° C. for 12 hours, in which (FIG. 9A) is a graph showing the voltage-current density characteristics of the device, and (FIG. 9B) is a graph showing the current density-external quantum efficiency characteristics of the device.

FIGS. 10A and 10B This includes graphs each showing device characteristics of the organic electroluminescent device produced in Comparative Example 2, as measured before and after heating the device at 80° C. for 12 hours, in which (FIG. 10A) is a graph showing the voltage-current density characteristics of the device, and (FIG. 10B) is a graph showing the current density-external quantum efficiency characteristics of the device.

### DESCRIPTION OF EMBODIMENTS

The contents of the invention will be described in detail below. The constitutional elements may be described below with reference to representative embodiments and specific examples of the invention, but the invention is not limited to the embodiments and the examples. In the description, a numerical value range expressed using “A to B” denotes a range including numerical values before and after “to” as a minimum value and a maximum value, respectively. The hydrogen atom that is present in a molecule of the compound used in the invention is not particularly limited in isotope species, and for example, all the hydrogen atoms in the molecule may be ¹H, and all or a part of them may be ²H (deuterium D).

### [Compound Represented by General Formula (1)]

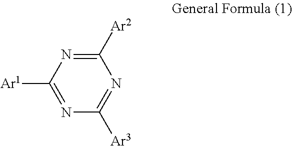

In the formula (1), Ar¹ to Ar³ each independently represent a substituted or unsubstituted aryl group or a substituted or unsubstituted heteroaryl group.

All of Ar¹ to Ar³ may be a substituted or unsubstituted aryl group, or all of them may be a substituted or unsubstituted heteroaryl group, or two of Ar¹ to Ar³ may be a substituted or unsubstituted aryl group and the remaining one may be a substituted or unsubstituted heteroaryl group, or two of Ar¹ to Ar³ may be a substituted or unsubstituted heteroaryl group and the remaining one may be a substituted or unsubstituted aryl group.

In the following description, the “aryl group” in the substituted or unsubstituted aryl group that Ar¹ to Ar³ represent, that is, the aryl group bonding to the triazine ring of the general formula (1) is referred to as “the aryl group in Ar¹ to Ar³”, and the “heteroaryl group” in the substituted or unsubstituted heteroaryl group that Ar¹ to Ar represent, that is, the heteroaryl group bonding to the triazine ring of the general formula (1) is referred to as “the heteroaryl group in Ar¹ to Ar³”, and these may be collectively referred to as “the aryl group or the heteroaryl group in Ar¹ to Ar³”.

In the general formula (1), at least one of Ar¹ to Ar³ contains a skeleton represented by the general formula (2) to be mentioned hereinunder. At least one of Ar¹ to Ar³ may be a group (heteroaryl group) bonding at any one bonding position of R¹ to R⁸ in the general formula (2), and in this case, the dibenzofuran ring or the dibenzothiophene ring directly bonds to the triazine ring in the general formula (1). At least one of Ar¹ to Ar³ may bond to the triazine ring in the general formula (1) via the group that any one of R¹ to R⁸ in the general formula (2) represents. At this time, at least one of Ar¹ to Ar³ is preferably an aryl group substituted with a group containing a skeleton represented by the general formula (2), or a heteroaryl group substituted with a group containing a skeleton represented by the general formula (2). At least one of Ar¹ to Ar³ may have such a structure that the skeleton represented by the general formula (2) is condensed with a hydrocarbon ring or a hetero ring.

Ar¹ to Ar³ do not contain a 4-(benzofuran-1-yl)carbazol-9-yl group or a 4-(benzothiophen-1-yl)carbazol-9-yl group having the structure mentioned below. In the following structure, * represents a bonding position. Preferably, the compound represented by the general formula (1) does not contain a 4-(benzofuran-1-yl)carbazole skeleton or a 4-(benzothiophen-1-yl)carbazole skeleton.

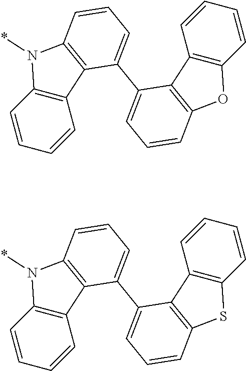

All of Ar¹ to Ar³ may contain the skeleton represented by the general formula (2), or two of Ar¹ to Ar³ may contain the skeleton represented by the general formula (2), or only one of Ar¹ to Ar³ may contain the skeleton represented by the general formula (2). At least one of Ar¹ to Ar³ may contain only one skeleton represented by the general formula (2), or may contain 2 or more skeletons represented by the general formula (2). For example, all of Ar¹ to Ar³ may contain 2 or more skeletons represented by the general formula (2), or two of Ar¹ to Ar³ may contain 2 or more skeletons represented by the general formula (2), or only one of Ar¹ to Ar³ may contain 2 or more skeletons represented by the general formula (2). In the case where 2 or more of Ar¹ to Ar³ contain a skeleton represented by the general formula (2), the groups that contain the skeleton represented by the general formula (2) may be the same as or different from each other, but are preferably the same.

The aryl group referred to in this description may be a group composed of only one aromatic hydrocarbon ring, or may be a group of an aromatic hydrocarbon ring condensed with one or more rings. In the case where the group of an aromatic hydrocarbon ring condensed with one or more rings, the group employable herein may be a group of an aromatic hydrocarbon ring condensed with one or more of an aromatic hydrocarbon ring, an aliphatic hydrocarbon ring and a non-aromatic hetero ring. The carbon number of the aryl group may be, for example, 6 or more, 10 or more, 14 or more, or 18 or more. The carbon number thereof may be 30 or less, 18 or less, 14 or less, or 10 or less. Specific examples of the aryl group include a phenyl group, a 1-naphthyl group, a 2-naphthyl group, a 1-anthracenyl group, a 2-anthracenyl group, a 9-anthracenyl group, a 1-carbazolyl group, a 2-carbazolyl group, a 3-carbazolyl group, and a 4-carbazolyl group. A preferred example of the aryl group employable for Ar¹ to Ar³ is a substituted or unsubstituted phenyl group.

The heteroaryl group referred to in this description may be a group composed of only one heteroaromatic ring, or may be a group of a heteroaromatic ring condensed with one or more rings. In the case of the group of a heteroaromatic ring condensed with one or more rings, the group employable herein may be a group of a heteroaromatic hydrocarbon ring condensed with one or more of an aromatic hydrocarbon ring, a heteroaromatic ring, an aliphatic hydrocarbon ring and a non-aromatic hetero ring. The ring skeleton constituent carbon number of the heteroaryl group may be, for example, 5 or more, 6 or more, 10 or more, 14 or more, or 18 or more. The carbon number thereof may be 30 or less, 18 or less, 14 or less, or 10 or less. The heteroaryl group may be a group bonding via the hetero atom thereof, or may be a group bonding via the carbon atom constituting the heteroaromatic ring. The heteroaromatic ring that constitutes the heteroaryl group for Ar¹ to Ar³ is preferably a 5-membered ring, a 6-membered ring, or a condensed ring having a structure of one or more 5-membered rings and one or more 6-membered rings. Preferably, the hetero atom constituting the ring skeleton of the heteroaromatic ring includes a nitrogen atom, an oxygen atom, and a sulfur atom, more preferably a nitrogen atom and an oxygen atom, and even more preferably a nitrogen atom. The number of the hetero atoms constituting the ring skeleton of the heteroaromatic ring is preferably 1 to 3, more preferably 1 or 2. Specific examples of the heteroaromatic ring include a pyridine ring, a pyridazine ring, a pyrimidine ring, a pyrazine ring, a pyrrole ring, a pyrazole ring, an imidazole ring, and a carbazol ring. Above all, a pyridine ring, a pyridazine ring, a pyrimidine ring, a pyrazine ring, an imidazole ring and a carbazole ring are preferred, and a carbazole ring is especially preferred. Also preferably, the heteroaromatic ring is a condensed ring having such a structure that the skeleton represented by the following general formula (2) is condensed with a hydrocarbon ring or a hetero ring. In this case, the condensed ring may bond to the triazine ring of the general formula (1) at the bonding position of any of R¹ to R⁸ of the skeleton represented by the general formula (2) via a single bond therebetween, or may bond to the triazine ring of the general formula (1) at a bondable position of the hydrocarbon ring or the hetero ring condensed with the skeleton represented by the general formula (2). An especially preferred example of the heteroaryl group is a heteroaryl group formed of a carbazole ring (that is, a carbazolyl group), and a carbazol-9-yl group is most preferred.

In one preferred embodiment of the present invention, at least one of Ar¹ to Ar³ is an aryl group substituted with a group containing a skeleton represented by the following general formula (2), a heteroaryl group substituted with a group containing a skeleton represented by the general formula (2), or a heteroaryl group having such a structure that a skeleton represented by the following general formula (2) is condensed with a hydrocarbon ring or a hetero ring. Regarding the specific examples and the preferred range of the aryl group and the heteroaryl group, reference may be made to the description of the specific examples and the preferred range of the aryl group and the heteroaryl group in “a substituted or unsubstituted aryl group or a substituted or unsubstituted heteroaryl group” described hereinabove.

Among Ar¹ to Ar³, the number of the aryl group substituted with a group containing a skeleton represented by the following general formula (2), the heteroaryl group substituted with a group containing a skeleton represented by the general formula (2), or the heteroaryl group having such a structure that a skeleton represented by the following general formula (2) is condensed with a hydrocarbon ring or a hetero ring may be one, or may be 2 or 3, but is preferably 1 or 2. In the case where 2 or 3 of Ar¹ to Ar³ are an aryl group substituted with a group containing a skeleton represented by the following general formula (2), a heteroaryl group substituted with a group containing a skeleton represented by the general formula (2), or a heteroaryl group having such a structure that a skeleton represented by the following general formula (2) is condensed with a hydrocarbon ring or a hetero ring, they may be the same as or different from each other, but are preferably the same. In the case where they are different from each other, the group containing a skeleton represented by the general formula (2) may differ, or the aryl group or the heteroaryl group substituted with a group containing a skeleton represented by the general formula (2) may differ, or the hydrocarbon ring or the hetero ring condensed with a skeleton represented by the general formula (2) may differ.

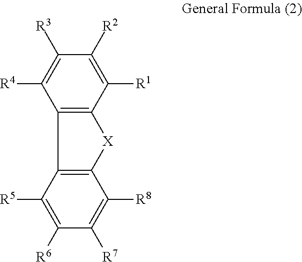

In the general formula (2), X represents O or S. When X is 0, the ring skeleton in the general formula (2) is a dibenzofuran skeleton, and when X is S, the ring skeleton in the general formula (2) is a dibenzothiophene skeleton.

R¹ to R⁸ each independently represent a hydrogen atom, a substituent or a bonding position.

Here, the “bonding position” of R¹ to R⁸ means a bonding position at which the skeleton represented by the general formula (2) bonds to the aryl group substituted with a group containing a skeleton represented by the general formula (2) or to the heteroaryl group substituted with a group containing a skeleton represented by the general formula (2), via a single bond therebetween, or a means a bonding position at which the skeleton represented by the general formula (2) bonds to a divalent linking group to be mentioned below, which the group containing a skeleton represented by the general formula (2) contains, (a divalent linking group that links the skeleton represented by the general formula (2) to the aryl group or the heteroaryl group of Ar¹ to Ar³), via a single bond therebetween. Also the bonding position means a bonding position at which the skeleton represented by the general formula (2) bonds to the triazine ring in the general formula (1) via a single bond therebetween. The group containing a skeleton represented by the genera formula (2) is preferably a group bonding to any one bonding position of R¹ to R⁸, more preferably a group bonding to any one bonding position of R¹ or R⁴, even more preferably a group bonding to the aryl group or the heteroaryl group in Ar¹ to Ar³ at any one bonding position of R¹ to R⁸, via a single bond therebetween, and further more preferably a group bonding to the aryl group or the heteroaryl group in Ar¹ to Ar³ at a bonding position of R¹ or R⁴, via a single bond therebetween.

Regarding the remaining positions of R¹ to R⁸ except the bonding position in the skeleton represented by the general formula (2), all of the remaining positions may be substituents or a part thereof may be substituents and the still remaining ones may be hydrogen atoms, or all of the remaining positions may be hydrogen atoms, but preferably, a part of the remaining positions are substituents and the still remaining ones are hydrogen atoms, or all of the remaining positions are hydrogen atoms, and more preferably, all of the remaining positions are hydrogen atoms.

Specific examples of the substituent that R¹ to R⁸ may have include a hydroxy group, a halogen atom, a cyano group, an alkyl group, an alkoxy group, a thioalkoxy group, a secondary amino group, a tertiary amino group, an acyl group, an aryl group, a heteroaryl group, an aryloxy group, a heteroaryloxy group, a thioaryloxy group, a thioheteroaryloxy group, an alkenyl group, an alkynyl group, an alkoxycarbonyl group, an alkylsulfonyl group, a haloalkyl group, an alkylamide group, an arylamide group, a silyl group, a trialkylsilylalkyl group, a trialkylsilylalkenyl group, a trialkylsilylalkynyl group, and a nitro group. Among these specific examples, substitutable ones may be further substituted with a substituent. More preferred substituents include a substituted or unsubstituted alkyl group, a substituted or unsubstituted alkoxy group, a substituted or unsubstituted thioalkoxy group, a substituted or unsubstituted aryl group, a substituted or unsubstituted heteroaryl group, a substituted or unsubstituted aryloxy group, a substituted or unsubstituted heteroaryloxy group, a substituted or unsubstituted thioaryloxy group, a substituted or unsubstituted thioheteroaryloxy group, a secondary amino group, a tertiary amino group, and a substituted or unsubstituted silyl group. Even more preferred substituents include a a substituted or unsubstituted alkyl group, a substituted or unsubstituted alkoxy group, a substituted or unsubstituted aryl group, and a substituted or unsubstituted heteroaryl group. Regarding the carbon number of these substituents, the carbon number of the substituted or unsubstituted alkyl group is preferably 1 to 20, more preferably 1 to 10, even more preferably 1 to 5, the carbon number of the substituted or unsubstituted alkoxy group and the substituted or unsubstituted thioalkoxy group is preferably 1 to 20, the carbon number of the substituted or unsubstituted aryl group, the substituted or unsubstituted aryloxy group and the substituted or unsubstituted thioaryloxy group is preferably 6 to 40, the carbon number of the substituted or unsubstituted heteroaryl group, the substituted or unsubstituted heteroaryloxy group and the substituted or unsubstituted thioheteroaryloxy group is preferably 3 to 40, the carbon number of the secondary amino group and the tertiary amino group is preferably 1 to 20, the carbon number of the silyl group substituted with an alkyl group is preferably 3 to 20. In the case where each substituent is further substituted with a substituent (for example, in the case of a substituted alkyl group), the carbon number thereof means a total carbon number including the carbon number of the substituted substituent and the carbon number of the substituent with which the substituent is substituted.

The halogen atom referred to in this description includes a fluorine atom, a chlorine atom, a bromine atom, and an iodine atom.

The alkyl group referred to in this description may be linear, branched or cyclic. The group may contain two or more linear moieties, cyclic moieties and/or branched moieties. The carbon number of the alkyl group may be, for example, 1 or more, 2 or more, 4 or more, 6 or more. The carbon number thereof may be 30 or less, 20 or less, 10 or less, 6 or less, or 4 or less. Specific examples of the alkyl group include a methyl group, an ethyl group, an n-propyl group, an isopropyl group, an n-butyl group, an isobutyl group, a tert-butyl group, an n-pentyl group, an isopentyl group, an n-hexyl group, an isohexyl group, a 2-ethylhexyl group, an n-heptyl group, an isoheptyl group, an n-octyl group, an isooctyl group, an n-nonyl group, an isononyl group, an n-decanyl group, an isodecanyl group, a cyclopentyl group, a cyclohexyl group, and a cycloheptyl group.

The alkenyl group referred to in this description may be linear, branched or cyclic. The group may contain two or more linear moieties, cyclic moieties and/or branched moieties. The carbon number of the alkenyl group may be, for example, 2 or more, 4 or more, or 6 or more. The carbon number thereof may be 30 or less, 20 or less, 10 or less, 6 or less, or 4 or less. Specific examples of the alkenyl group include an ethenyl group, an n-propenyl group, an isopropenyl group, an-butenyl group, an isobutenyl group, a tert-butenyl group, an n-pentenyl group, an isopentenyl group, an n-hexenyl group, an isohexenyl group, a 2-ethylhexenyl group, an n-heptenyl group, an isoheptenyl group, an n-octenyl group, an isooctenyl group, an n-nonenyl group, an isononenyl group, an n-decenyl group, an isodecenyl group, a cyclopentenyl group, a cyclohexenyl group, and a cycloheptenyl group.

The alkynyl group referred to in this description may be linear, branched or cyclic. The group may contain two or more linear moieties, cyclic moieties and/or branched moieties. The carbon number of the alkynyl group may be, for example, 2 or more, 4 or more, or 6 or more. The carbon number thereof may be 30 or less, 20 or less, 10 or less, 6 or less, or 4 or less. Specific examples of the alkenyl group include an ethynyl group, an n-propynyl group, an isopropynyl group, an n-butynyl group, an isobutynyl group, a tert-butynyl group, an n-pentynyl group, an isopentynyl group, an n-hexynyl group, an isohexynyl group, a 2-ethylhexynyl group, an n-heptynyl group, an isoheptynyl group, an n-octynyl group, an isooctynyl group, an n-nonynyl group, an isononynyl group, an n-decynyl group, an isodecynyl group, a cyclohexynyl group and a cycloheptynyl group.

Regarding the description and specific examples of the alkyl moiety of the alkoxy group referred to herein, the description and specific examples of the alkyl moiety of the thioalkoxy group referred to herein, the description and specific examples of the alkyl moiety of the alkylthio group referred to herein, the description and specific examples of the alkyl moiety of the secondary amino group or the tertiary amino group of an alkylamino group referred to herein, the description and specific examples of the alkyl moiety of the acyl group (the remaining moiety of the acyl group after removal of the carbonyl group therefrom) referred to herein, the description and specific examples of the alkyl moiety of the alkoxycarbonyl group referred to herein, the description and specific example of the alkyl moiety of the alkylsulfonyl group referred to herein, the description and specific examples of the alkyl moiety of the haloalkyl group referred to herein, the description and specific example of the alkyl group of the alkylamide group referred to herein, the description and specific example of the alkyl moiety of the silyl group of an alkylsilyl group referred to herein, the description and specific examples of each alkyl moiety of the trialkylsilylalkyl group referred to herein, the description and specific examples of the alkyl moiety of the trialkylsilylalkenyl group referred to herein, and the description and specific examples of the alkyl moiety of the trialkylsilylalkynyl group referred to herein, reference may be made to the description and specific examples of the alkyl group given hereinabove.

Regarding the description and specific examples of the aryl moiety of the secondary amino group or the tertiary amino group of an arylamino group referred to herein, the description and specific examples of the aryl moiety of the aryloxy group referred to herein, the description and specific examples of the aryl moiety of the thioaryloxy group referred to herein, and the description and specific examples of the silyl group or an arylsilyl group referred to herein, reference may be made to the description and specific examples of the aryl group given hereinabove.

Regarding the description and specific examples of the heteroaryl moiety of the secondary amino group and the tertiary amino group or a heteroarylamino group referred to herein, the description and specific examples of the heteroaryl moiety of the heteroaryloxy group referred to herein, the description and specific examples of the heteroaryl moiety of the thioheteroaryloxy group referred to herein, and the description and specific examples of the heteroaryl moiety of the silyl group of a heteroarylsilyl group referred to herein, reference may be made to the description and specific examples of the aryl group given hereinabove.

Regarding the description and specific examples of the alkenyl moiety of the trialkylsilylalkenyl group referred to herein, reference may be made to the description and specific examples of the alkenyl group given hereinabove.

Regarding the description and specific examples of the alkynyl moiety of the trialkylsilylalkynyl group referred to herein, reference may be made to the description and specific examples of the alkynyl group given hereinabove.

R¹ and R², R² and R³, R³ and R⁴, R⁵ and R⁶, R⁶ and R⁷, and R⁷ and R⁸ each may bond to each other to form a cyclic structure. The cyclic structure may be an aromatic ring or an aliphatic ring, or may contain a hetero atom. Further, the cyclic structure may be a condensed ring of 2 or more rings. The hetero atom as referred to herein is preferably one selected from the group consisting of a nitrogen atom, an oxygen atom and a sulfur atom. Examples of the cyclic structure to be formed include a benzene ring, a naphthalene ring, a pyridine ring, a pyridazine ring, a pyrimidine ring, a pyrazine ring, pyrrole ring, an imidazole ring, a pyrazole ring, a triazole ring, an imidazoline ring, an oxazole ring, an isoxazole ring, a thiazole ring, an isothiazole ring, an indole ring, a cyclohexadiene ring, a cyclohexene ring, a cyclopentaene ring, a cycloheptatriene ring, a cycloheptadiene ring, and a cycloheptaene ring. Preferred are a pyrrole ring and an indole ring, and more preferred is an indole ring. In the case where R¹ to R⁸ in the skeleton represented by the general formula (2) bond to each other to form a cyclic structure, the bond thereof to the aryl group or the heteroaryl group may be a bond at the bonding position of any of R¹ to R⁸ in the skeleton represented by the general formula (2), or may be a bond that bonds at a bondable position of the cyclic structure formed by bonding of R¹ to R⁸ to each other; however, in the case where the cyclic structure formed by bonding of R¹ to R⁸ to each other is a pyrrole ring or an indole ring, preferably, the cyclic structure bonds to the aryl group or the heteroaryl group at the nitrogen atom thereof. Hereinunder shown are specific examples of a group containing a skeleton represented by the general formula (2) where R¹ and R², or R³ and R⁴ each bond to each other to form an indole ring. In this, * indicates a bonding position. However, the group containing a skeleton represented by the general formula (2) that can be employed in the compounds of the present invention is not whatsoever limitatively interpreted by these specific examples.

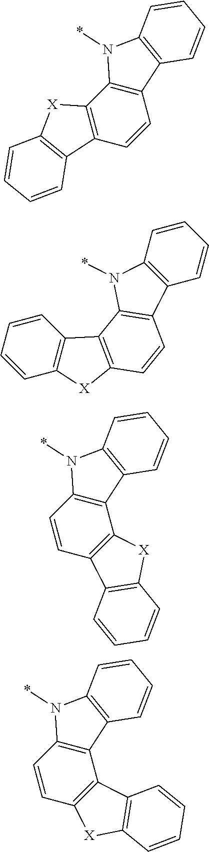

In the above-mentioned formulae, X represents O or S. The single bond from N bonds to the aryl group or the heteroaryl group in Ar¹ to Ar³ in the general formula (1). The methine group may be substituted with a substituent.

The number of the skeletons represented by the general formula (2) existing inside the molecule of the compound represented by the general formula (1) may be 1 or 2 or more, but is preferably 2 or more, more preferably 2 to 6, even more preferably 2 or 3, and especially preferably 2. In the case where the compound represented by the general formula (1) has 2 or more skeletons represented by the general formula (2) in the molecule thereof, the skeletons may be the same or different. In the case where the skeletons differ, X may differ, or R¹ to R⁸ may differ. Preferably, two or more skeletons represented by the general formula (2) in the molecule of the compound are all the same.

The group containing a skeleton represented by the general formula (2) may be composed of the skeleton represented by the general formula (2) alone, or may contain any other group. The other group includes a divalent linking group that links the skeleton represented by the general formula (2) to the aryl group or the heteroaryl group in Ar¹ to Ar³, and a divalent linking group that links to the triazine ring of the general formula (1). The linking group bonds to the skeleton represented by the general formula (2) at any one bonding position of R¹ to R⁸, via a single bond therebetween, and bonds to the bondable position of the aryl group, the heteroaryl group or the triazine ring, and the group may be formed of a single atom, or may be composed of an atomic group. Preferably, the group is composed of an atomic group. The linking group composed of an atomic group is preferably a linking group of an aromatic ring, more preferably a linking group of a heteroaromatic ring, and even more preferably a linking group of a carbazole ring. A substitutable position of the linking group may be substituted with a substituent.

The group containing a skeleton represented by the general formula (2) and a linking group includes a group represented by the following general formula (3).

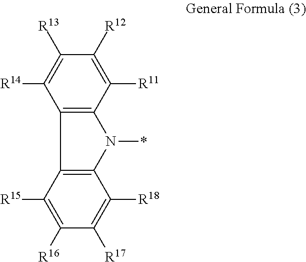

In the general formula (3), * represents a bonding position to the aryl group or the triaryl group in Ar¹ to Ar³ or to the triazine ring in the general formula (1). R¹¹ to R¹⁸ each independently represent a hydrogen atom or a substituent, at least one of R¹¹ to R¹⁸ is a skeleton represented by the general formula (2) and bonding to the carbazole ring of the general formula (3) at the bonding position of any one of R¹ to R⁸ via a single bond therebetween. R¹¹ and R¹², R¹² and R¹³, R¹³ and R¹⁴, R¹⁵ and R¹⁶, R¹⁶ and R¹⁷, and R¹⁷ and R¹⁸ each may bond to each other to form a cyclic structure.

Regarding the specific examples and the preferred ranges of the substituents that R¹¹ to R¹⁸ may have, and the specific examples and the preferred ranges of the cyclic structure to be formed by a predetermined combination among R¹¹ to R¹⁸ each bonding to each other, reference may be made to the specific examples and the preferred ranges of the substituents and the cyclic structures described for R¹ to R⁸ given hereinabove.

Preferably, in the group represented by the general formula (3), one to four of R¹¹ to R¹⁸ are the skeleton represented by the general formula (2), and more preferably, one or two thereof are the skeleton represented by the general formula (2). Among R¹¹ to R¹⁸, preferably, at least one of R¹² to R¹⁷ is a skeleton represented by the general formula (2) and R¹¹ and R¹⁸ are a hydrogen atom. Among R¹¹ to R¹⁸, at least one of R¹¹ to R¹³ and R¹⁶ to R¹⁸ may be a skeleton represented by the general formula (2) and R¹⁴ and R¹⁵ may be a hydrogen atom, or may be any other substituent than the skeleton represented by the general formula (2). Preferably, at least one or more of R¹², R¹³, R¹⁶ and R¹⁷ are a skeleton represented by the general formula (2), and more preferably, one or both of R¹³ and R¹⁶ are a skeleton of the general formula (2).

In the aryl group substituted with a group containing a skeleton represented by the general formula (2) or the heteroaryl group substituted with a group containing a skeleton represented by the general formula (2), the number of the groups containing a skeleton represented by the general formula (2) is 1 or more, and is an integer not more than the largest number of the substituents with which the aryl group or the heteroaryl group may be substituted. The substitutable position of the group containing a skeleton represented by the general formula (2) includes, for example, the methine group (—CH═) constituting an aryl group, or the methine group (—CH═) or the amino group (—NH—) constituting a heteroaryl group. The number of the substituents containing a skeleton represented by the general formula (2) is preferably 1 to 4, more preferably 1 or 2. In particular, in the case where one of Ar¹ to Ar³ is an aryl group substituted with a group containing a skeleton represented by the general formula (2) or a heteroaryl group substituted with a group containing a skeleton represented by the general formula (2), the number of the substituents containing a skeleton represented by the general formula (2) in the group is preferably 1 or 2, and in the case where 2 or 3 of Ar¹ to Ar³ are an aryl group substituted with a group containing a skeleton represented by the general formula (2) or a heteroaryl group substituted with a group containing a skeleton represented by the general formula (2), the number of the substituents containing a skeleton represented by the general formula (2) in these groups is preferably 1.

The substituting position of the group containing a skeleton represented by the general formula (2) is not specifically limited, but in the case where the aryl group to be substituted is a phenyl group and where the number of the substituent is 1, the substituting position is preferably a meta-position or a para-position relative to the bonding position to the triazine ring of the general formula (1), and in the case where the aryl group to be substituted is a phenyl group and the number of the substituents is 2, preferably, the bonding positions are both the meta-positions relative to the bonding position to the triazine ring of the general formula (1). In the case where the heteroaryl group to be substituted is a carbazol-9-yl group, the bonding positions are preferably one of the 3-position and the 6-position, or both of the 3-position and the 6-position.

Among the substitutable positions of the aryl group substituted with a group containing a skeleton represented by the general formula (2) or the heteroaryl group substituted with a group containing a skeleton represented by the general formula (2), the positions not substituted with a group containing a skeleton represented by the general formula (2) may be substituted with any other substituent than the group containing a skeleton represented by the general formula (2), or may be unsubstituted, but preferably, at least a part thereof are unsubstituted, and more preferably all are unsubstituted. Regarding the specific examples and the preferred ranges of the substituents in the case where the positions are substituted, reference may be made to the specific examples and the preferred range of the substituents that R¹ to R⁸ may have as given hereinabove. Among these substituents, an alkyl group and a carbazolyl group are preferred. The carbon number of the alkyl group referred to herein is preferably 1 to 20, more preferably 1 to 10, even more preferably 1 to 5. The alkyl group may be linear, branched or cyclic, but is preferably linear or branched. Examples of the alkyl group include a methyl group, an ethyl group, an n-propyl group, an isopropyl group, an n-butyl group, an isobutyl group, and a tert-butyl group. The carbazolyl group is preferably a carbazol-9-yl group. The substituting position of the substituents is not specifically limited. In the case where the aryl group to be substituted is a phenyl group, the group is preferably substituted at two positions, and more preferably, in the case, the group is substituted at both the meta-positions or at the ortho-position and the meta-position relative to the bonding position to the triazine ring of the general formula (1).

The substitutable positions of the heteroaryl group having such a structure that the skeleton represented by the general formula (2) is condensed with a hydrocarbon ring or a hetero ring may be substituted with a substituent, or may be unsubstituted, but preferably, at least a part thereof are unsubstituted, and more preferably all are unsubstituted. Regarding the specific examples and the preferred ranges of the substituents in the case where the substitutable positions are substituted, reference may be made to the specific examples and the preferred ranges of the substituents that R¹ to R⁸ may have, as given hereinabove. The substituent with which the heteroaryl group may be substituted may be a group containing a skeleton represented by the general formula (2).

Among the aryl group or the heteroaryl group in Ar¹ to Ar³, the substitutable positions of any others than the aryl group substituted with a group containing a skeleton represented by the general formula (2) and the heteroaryl group substituted with a group containing a skeleton represented by the general formula (2) may be substituted with any other substituent that a group containing a skeleton represented by the general formula (2), and may be unsubstituted, but preferably at least a part thereof are unsubstituted, and more preferably all are unsubstituted. Regarding the specific examples and the preferred ranges of the substituted substituents, reference may be made to the specific examples and the preferred ranges of the substituents that t R¹ to R⁸ may have, as given hereinabove.

Preferred examples of a group of the compounds represented by the general formula (1) of the present invention include a group satisfying at least one of the following requirements (a) to (c), and a group satisfying all of the following requirements (a) to (c) as a group showing preferred characteristics.

<Requirement (a)>

When only one of Ar¹ to Ar³ in the general formula (1) is an aryl group substituted with a group containing a skeleton represented by the general formula (2) and the aryl group is a phenyl group substituted with only one group containing a skeleton represented by the general formula (2), and when the group containing a skeleton represented by the general formula (2) is a group represented by the following general formula (A), and among R^{12a} to R^{16a}, only one of R^{12a} to R^{14a} is a skeleton represented by the general formula (2), the phenyl group substituted with only one group containing a skeleton represented by the general formula (2) is further substituted with an alkyl group, or at least one of R^{11a} to R^{18a} is an alkyl group, or excepting for the case where the phenyl group substituted with only one group containing a skeleton represented by the general formula (2) is further substituted with an alkyl group, and where at least one of R^{11a} to R^{18a} is an alkyl group, the skeleton represented by the general formula (2) bonds to the carbazole ring in the general formula (A) at the bonding position of R² or R³ via a single bond therebetween.

<Requirement (b)>

When only one of Ar¹ to Ar³ in the general formula (1) is an aryl group substituted with a group containing a skeleton represented by the general formula (2) and the aryl group is a phenyl group substituted with only one group containing a skeleton represented by the general formula (2), and when the group containing the skeleton represented by the general formula (2) is a group represented by the general formula (A), and among R^{12a} to R^{16a}, R^{13a} and R^{16a} alone are a skeleton represented by the general formula (2),
the substituting position of the group containing the skeleton represented by the general formula (A) to the phenyl group is an ortho-position or a para-position relative to the bonding position of the triazine ring.

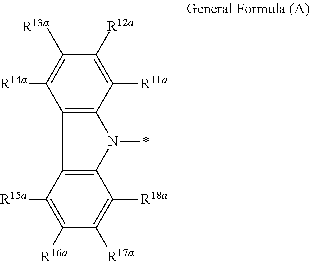

In the general formula (A), * represents a bonding position to the aryl group or the heteroaryl group of at least one of Ar¹ to Ar³ in the general formula (1). R^{11a} to R^{18a} each independently represent a hydrogen atom or a substituent, one or two of R^{12a} to R^{16a} is a skeleton represented by the general formula (2) and bonding to the carbazole ring at the bonding position of any one of R¹ to R⁸ via a single bond therebetween. However, among R^{12a} to R^{16a}, only one of R^{12a} to R₁₄ₐ or both R^{13a} and R^{16a} alone is/are a skeleton represented by the general formula (2). R^{11a} and R¹², R^{12a} and R¹³, R^{13a} and R^{14a}, R^{15a} and R^{16a}, R^{16a} and R^{17a}, and R^{17a} and R^{18a} each may bond to each other to form a cyclic structure.

The compound according to [34] or [35],

<Requirement (c)>

When only two of Ar¹ to Ar³ in the general formula (1) are an aryl group substituted with a group containing a skeleton represented by the general formula (2), and when the aryl group is a phenyl group to which only one skeleton represented by the general formula (2) bonds at the bonding position of R¹ via a single bond therebetween, R⁶ in the general formula (2) is not a pyrimidinyl group, and the bonding position to the phenyl group of the skeleton represented by the general formula (2) is an ortho-position or a meta-position relative to the bonding position of the triazine ring.

A group showing preferred characteristics among the compounds represented by the general formula (1) of the peent invention includes a compound group represented by the following general formula (4).

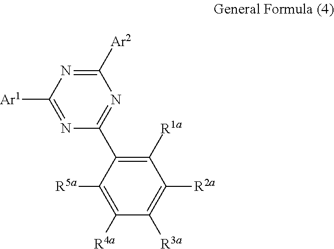

In the general formula (4), Ar¹ and Ar² each independently represent a substituted or unsubstituted aryl group or a substituted or unsubstituted heteroaryl group, R^{1a} to R^{5a} each independently represent a hydrogen atom or a substituent, at least one of R^{1a}, R^{3a} and R^{5a} contains a skeleton represented by the general formula (2). However, Ar¹, Ar² and R^{1a} to R^{5a} do not contain a 4-(benzofuran-1-yl)carbazole-9-yl group or a 4-(benzothiophen-1-yl)carbazol-9-yl group. R^{1a} and R^{2a}, R^{2a} and R^{3a}, R^{3a} and R^{4a}, and R^{4a} and R^{5a} each may independently bond to each other to form a cyclic structure.

Regarding the description, the preferred ranges and the specific examples of Ar¹ and Ar² in the general formula (4), reference may be made to the corresponding description of Ar¹ and Ar² in the general formula (1). Regarding the description, the preferred ranges and the specific examples of the substituents that R^{1a} to R^{5a} in the general formula (4) may have, reference may be made to the description of the substituents that R¹ to R⁸ may have.

Preferred embodiments include a case where R^{3a} in the general formula (4) contains a skeleton represented by the general formula (2), especially a case where R^{3a} in the general formula (4) contains a skeleton represented by the general formula (2) and R^{1a}, R^{2a}, R^{4a} and R^{5a} do not contain a skeleton represented by the general formula (2), and a case where Ar² in the general formula (4) contains a skeleton represented by the general formula (2), especially where Ar² in the general formula (2) has the same structure as the structure of

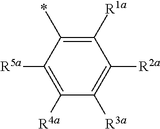

in the general formula (4), wherein * indicates a bonding position to the triazine ring.

Another group showing preferred characteristics among the compounds represented by the general formula (1) of the peent invention includes a compound group represented by the following general formula (5).

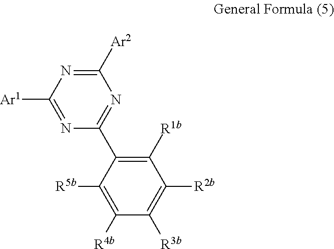

In the general formula (5), Ar¹ and Ar² each independently represent a substituted or unsubstituted aryl group or a substituted or unsubstituted heteroaryl group, R^{1b} to R^{5b} each independently represent a hydrogen atom or a substituent, and at least one of R^{1b}, R^{3b}, R^{4b} and R^{5b}, and R^{2b} each independently contain a skeleton represented by the general formula (2). However, Ar¹, Ar² and R^{1b} to R^{5b} do not contain a 4-(benzofuran-1-yl)carbazol-9-yl group or a 4-(benzothiophen-1-yl)carbazol-9-yl group. R^{1b} and R^{2b}, R^{2b} and R^{3b}, R^{3b} and R^{4b}, and R^{4b} and R^{5b} each may independently bond to each other to form a cyclic structure.

Regarding the description, the preferred ranges and the specific examples of Ar¹ and Ar² in the general formula (5), reference may be made to the corresponding description of Ar¹ and Ar² in the general formula (1). Regarding the description, the preferred ranges and the specific examples of the substituents that R^{1b} to R^{5b} in the general formula (5) may have, reference may be made to the description of the substituents that R¹ to R⁸ may have.

One preferred embodiment of a case where R^{4b} in the general formula (5) contains a skeleton represented by the general formula (2) includes a case where R^{2b} and R^{4b} in the general formula (5) are groups having the same structure.

Still another group showing preferred characteristics among the compounds represented by the general formula (1) of the peent invention includes a compound group represented by the following general formula (6).

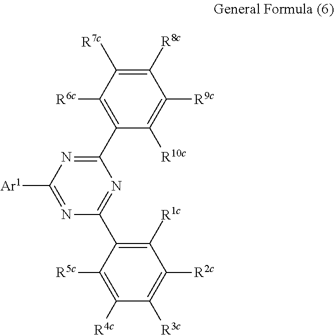

In the general formula (6), Ar¹ represents a substituted or unsubstituted aryl group or a substituted or unsubstituted heteroaryl group, R^{1c} to R^{10c} each independently represent a hydrogen atom or a substituent, but at least one of R^{6c} to R^{10c}, and R^{2c} each independently contain a skeleton represented by the general formula (2). However, R^{7c} in the case where only R^{2c} and R^{7c} among R^{1c} to R^{10c} contain a skeleton represented by the general formula (2) is not the same as R^{2c}, and in the case where R^{2c} contains a dibenzofuran ring, the group is not a group where the oxygen atom in the dibenzofuran ring is substituted with a sulfur atom, and in the case where R^{2c} contains a dibenzothiophene ring, the group is not a group where the sulfur atom in the dibenzothiophene ring is substituted with an oxygen atom. Ar¹, Ar² and R₁° to R^{1c} do not contain a 4-(benzofuran-1-yl)carbazol-9-yl group or a 4-(benzothiophen-1-yl)carbazol-9-yl group. R^{1c} and R^{2c}, R^{2c} and R^{3c}, R^{3c} and R^{4c}, R^{4c} and R^{5c}, R^{6c} and R^{7c}, R^{7c} and R^{8c}, R^{8c} and R^{9c}, and R^{9c} and R^{10c} each may independently bond to each other to form a cyclic structure.

Regarding the description, the preferred range and the specific examples of Ar¹ in the general formula (6), reference may be made to the corresponding description of Ar¹ in the general formula (1). Regarding the description, the preferred ranges and the specific examples of the substituents that R^{1c} to R^{10c} in the general formula (6) may have, reference may be made to the description of substituents that R¹ to R⁸ may have.

Preferred embodiments include a case where at least two of R^{1c} to R^{5c} and at least two of R^{6c} to R^{10c} in the general formula (6) each independently contain a skeleton represented by the general formula (2), and a case where R^{2c} in the general formula (5) is a group containing a benzofuran-x-yl group or a dibenzothiophen-x-yl group, at least one of R^{6b} to R^{10b} is a group containing a dibenzofuran-y-yl group or a dibenzothiophen-y-yl group, x and y each represent a number indicating the bonding position of the dibenzofuryl group or the dibenzothienyl group, and x and y are not the same.

Specific examples of the compounds represented by the general formula (1) are shown below. However, the compounds represented by the general formula (1) employable in the present invention are not whatsoever limitatively interpreted by the following specific examples.

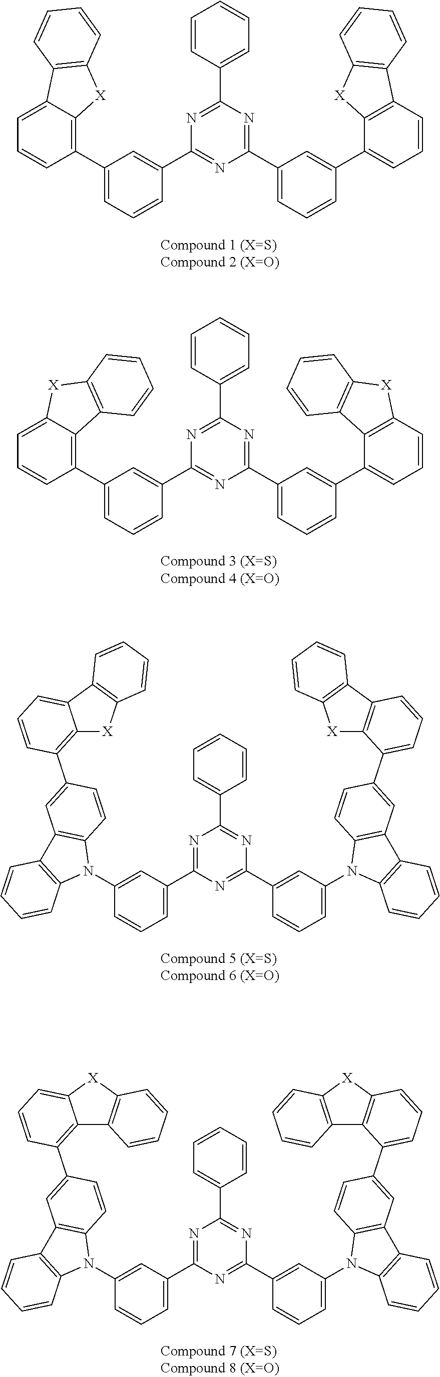

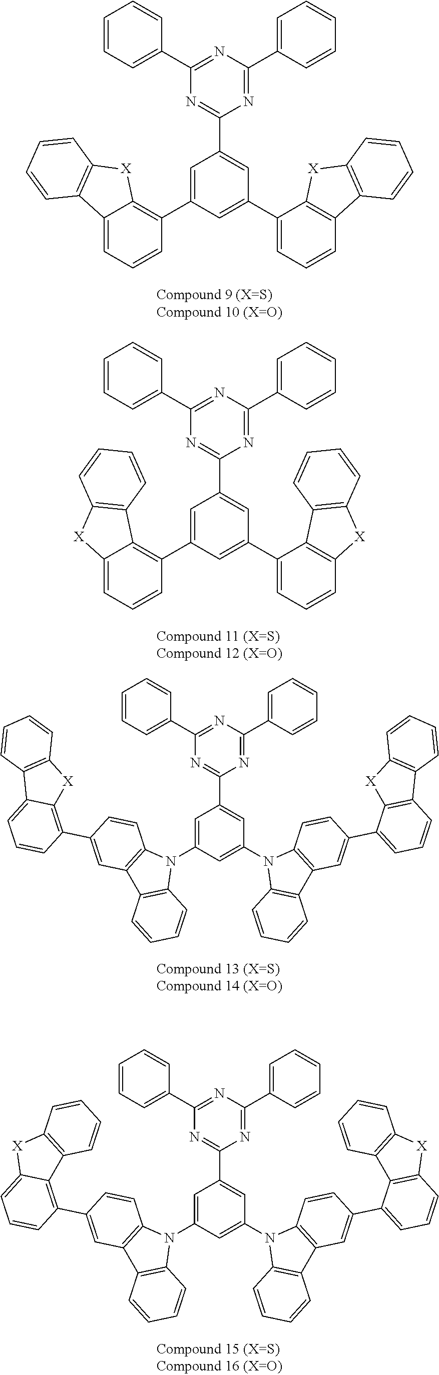

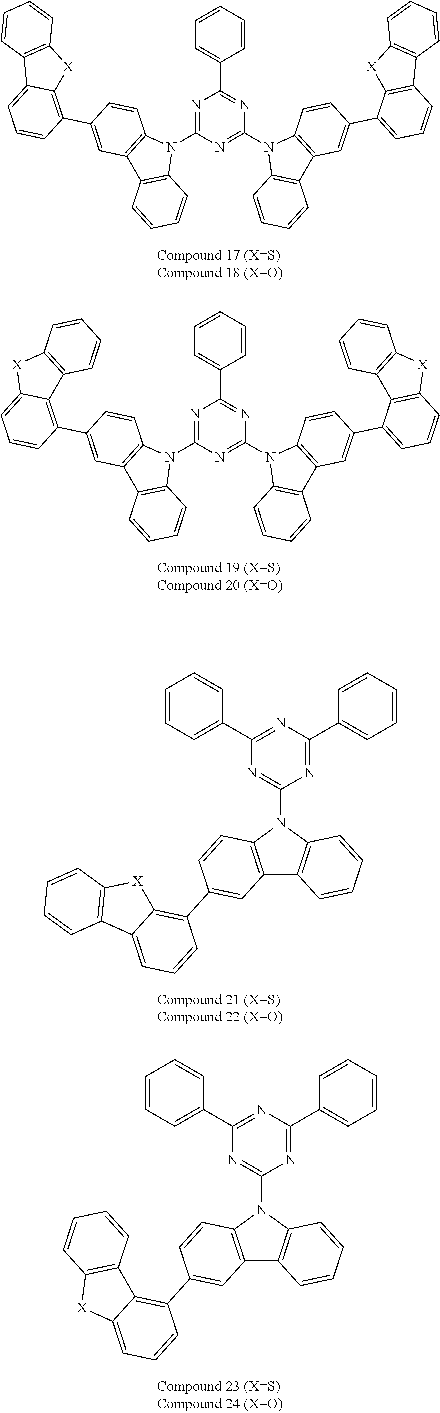

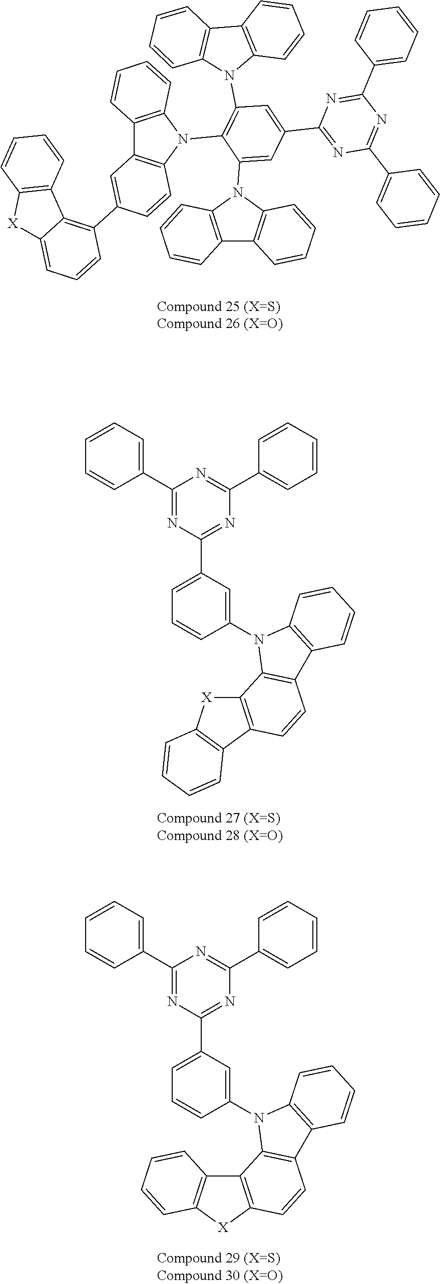

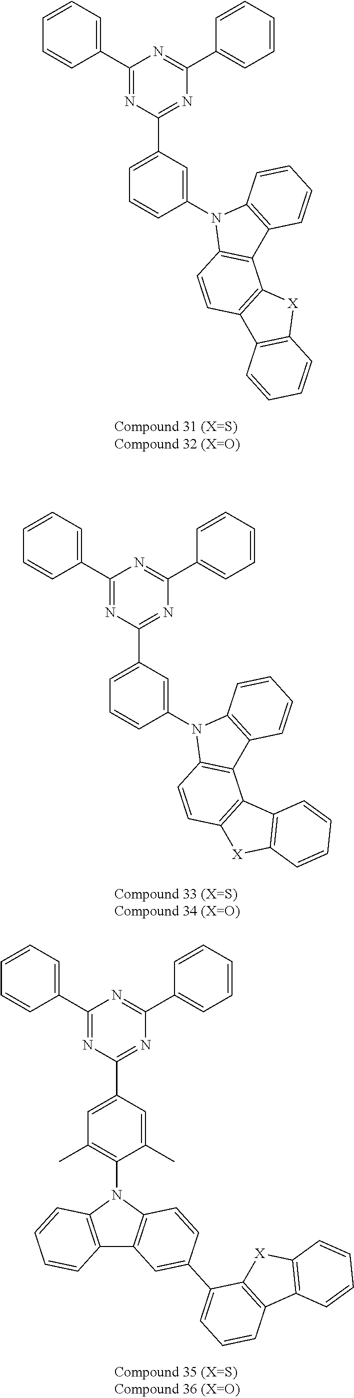

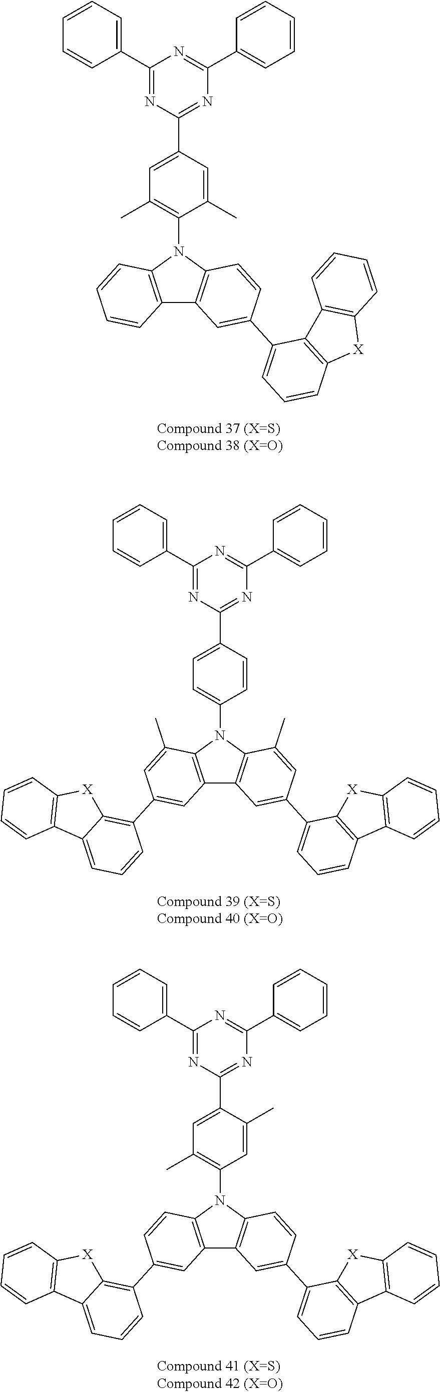

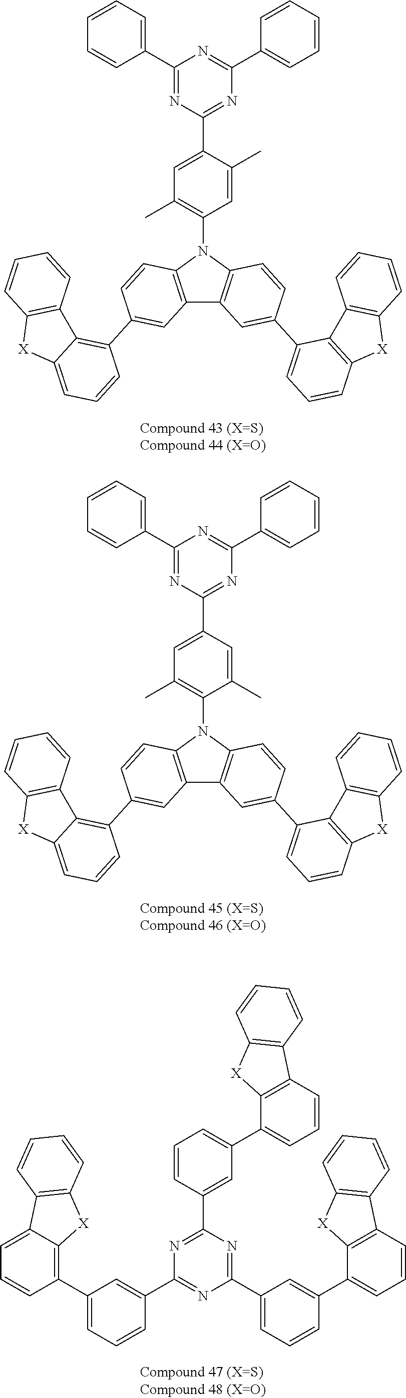

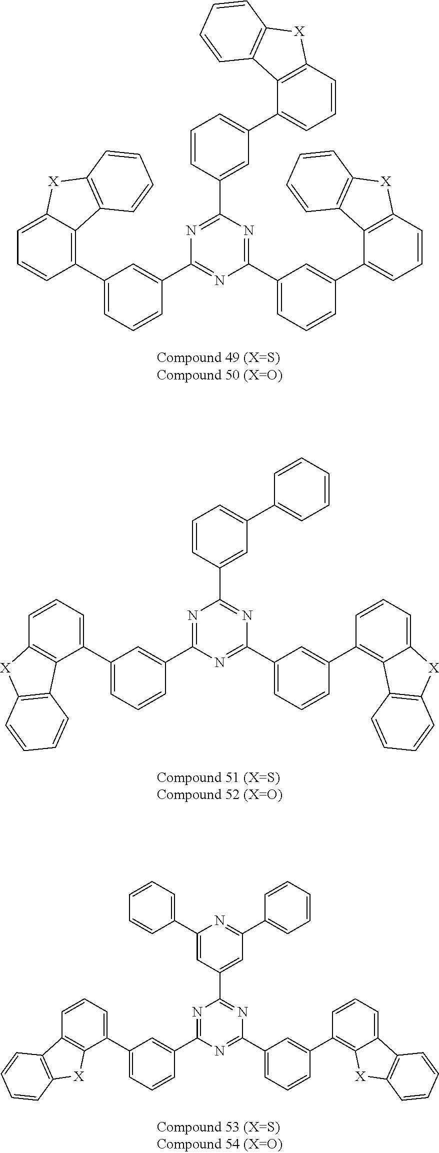

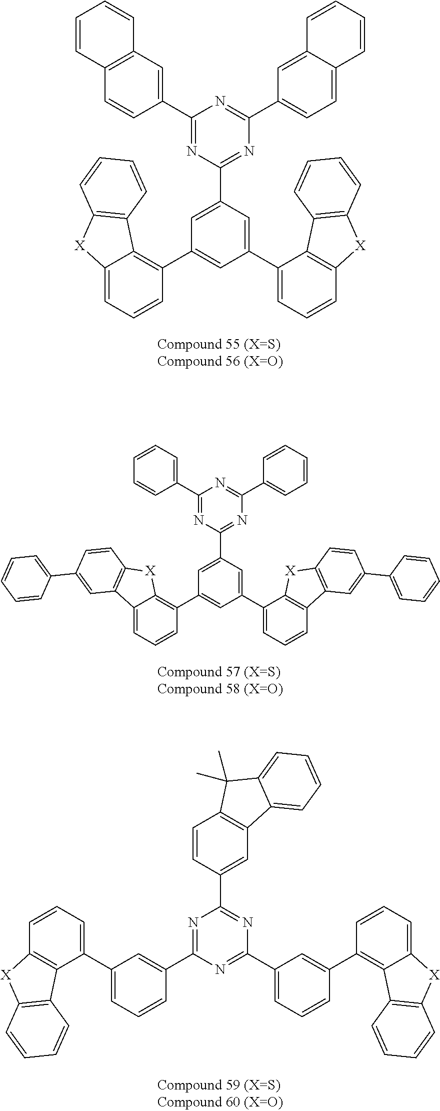

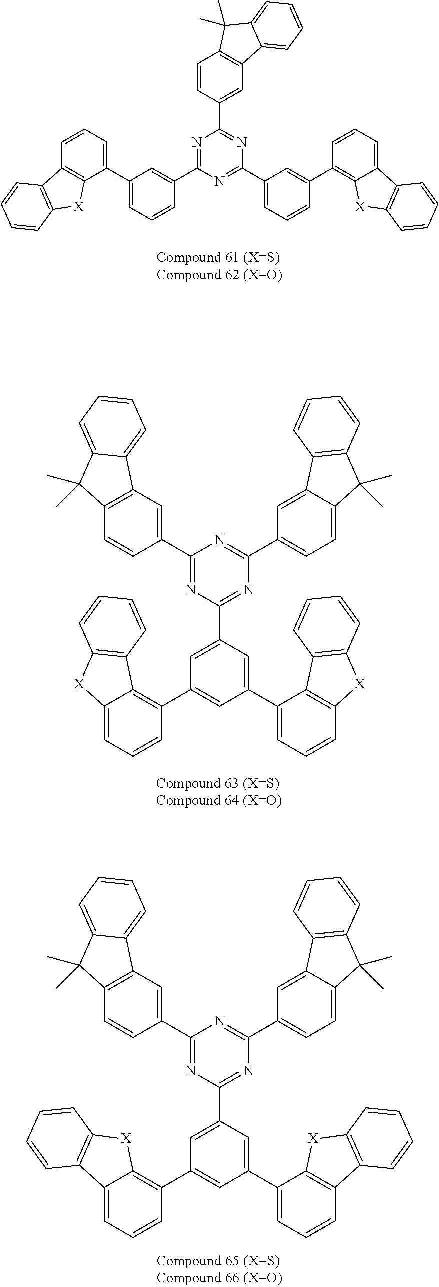

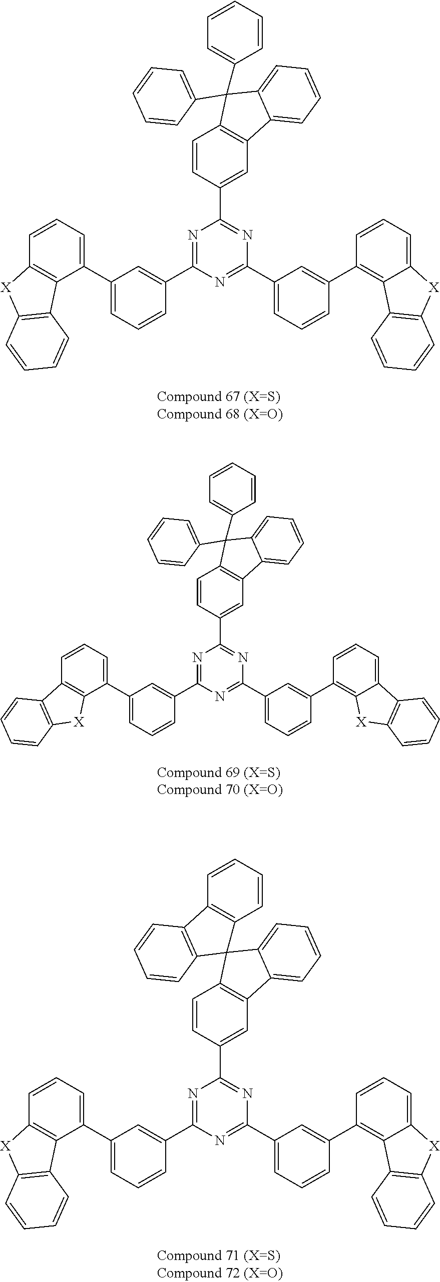

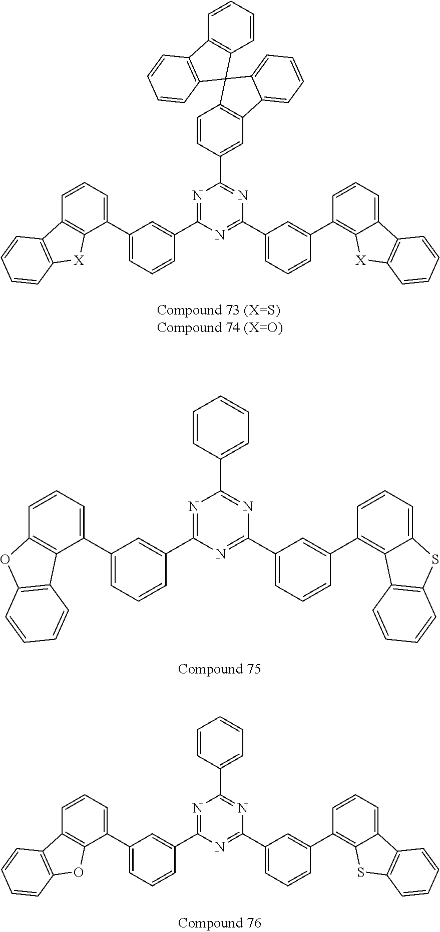

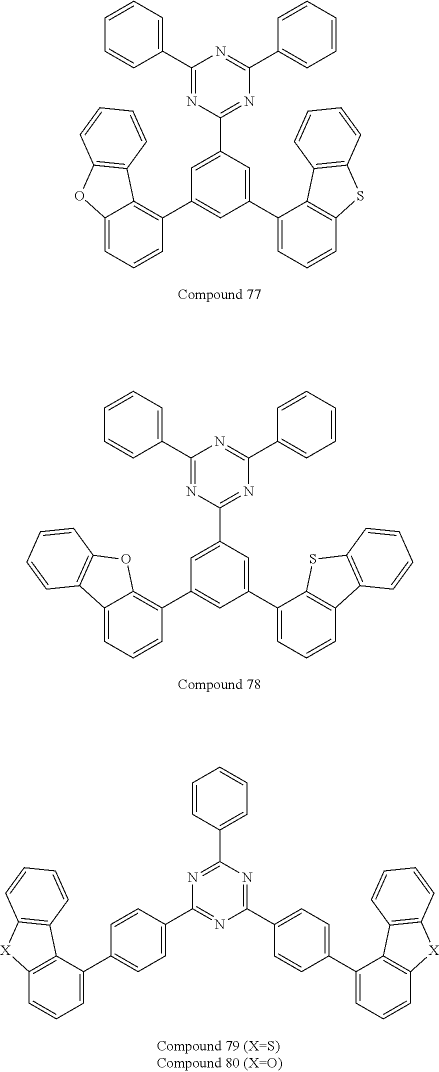

More detailed specific examples of the compounds represented by the general formula (1) are shown in the following Tables. In the Tables, the structures of Ar¹, Ar² and Ar³ are expressed as A1 to A6, L1 to L15, and B1 to B14.

In the Tables, the structures of A1 to A6 are as mentioned below. The mark *indicates the bonding position to the hydrazine ring in the general formula (1).

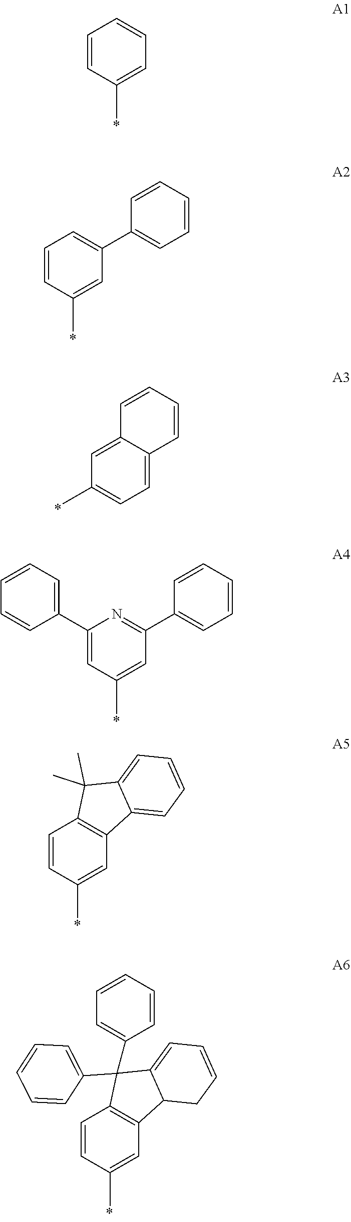

The structures of L1 to L15 in the Tables are as mentioned below. The mark *indicates the bonding position to the hydrazine ring in the general formula (1). Bn is any of the following B1 to B14 and is defined in the Tables. For example, “L1-B1” in the Tables means that Bn in the structure represented by the following L1 is BT.

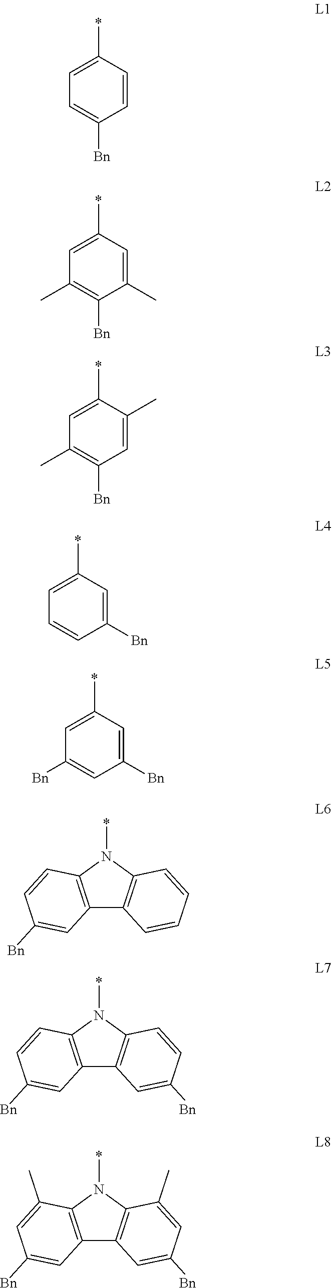

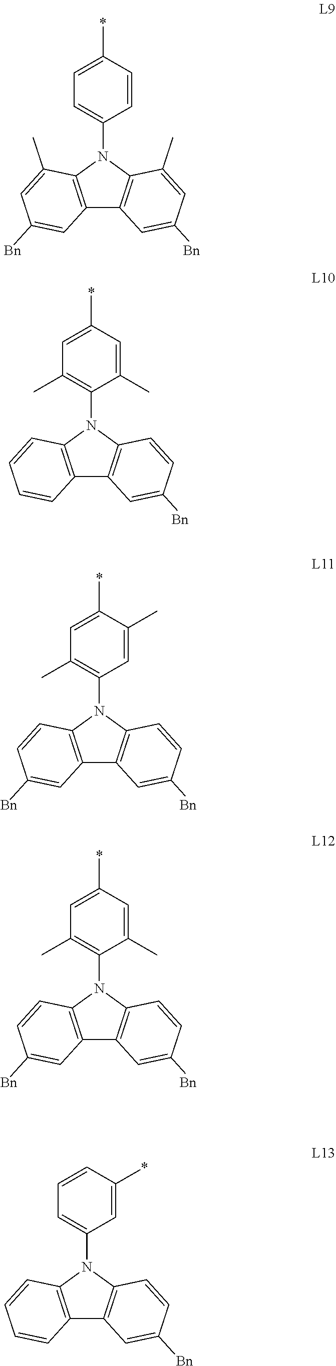

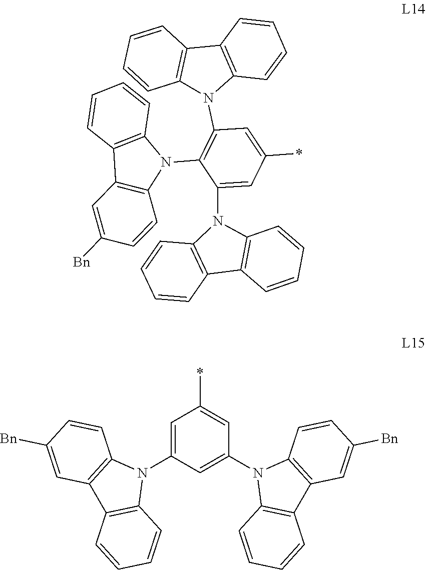

The structures of B1 to B14 in the Tab les are as mentioned below. The mark *indicates the bonding position to the hydrazine ring in the general formula (1), or the bonding position at the position of Bn in L1 to L15.

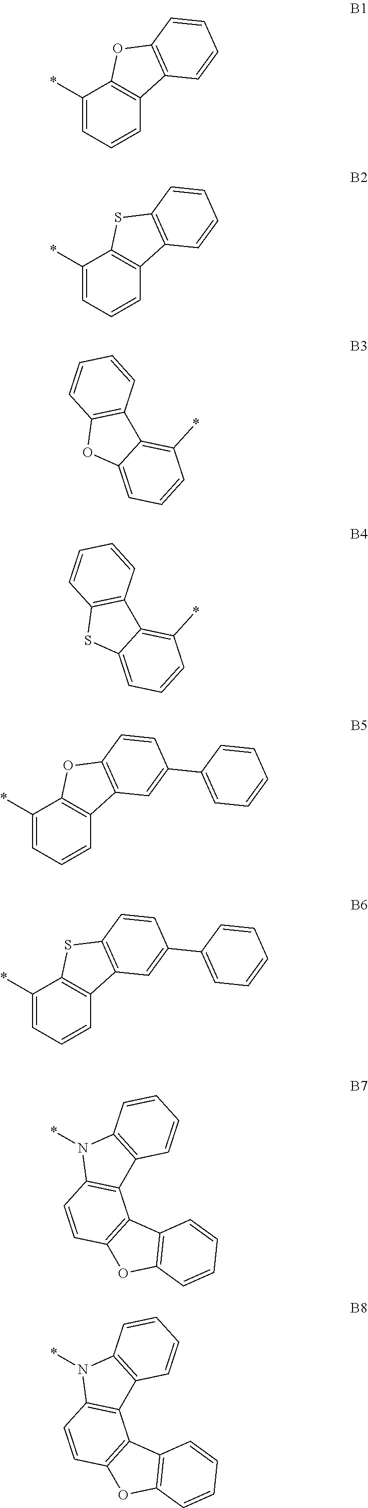

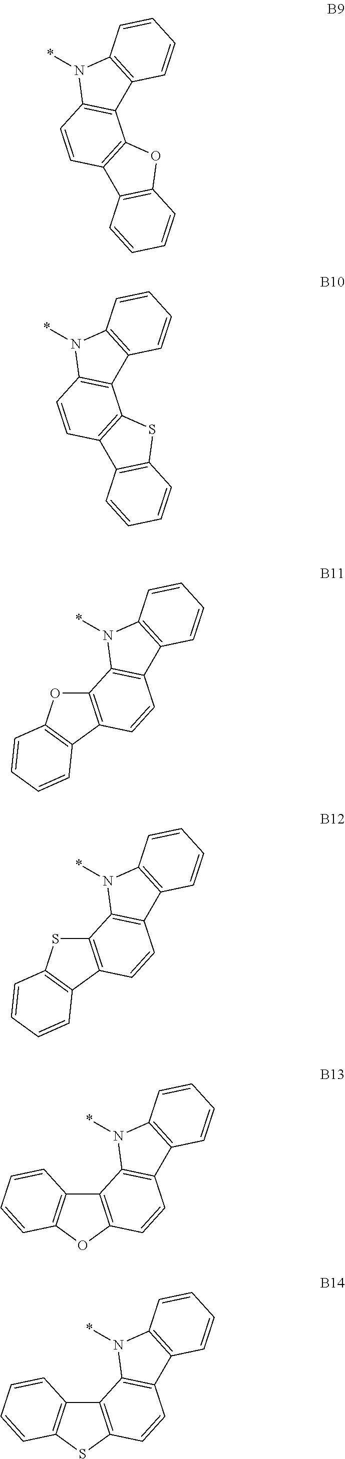

TABLE 1

| No. | Ar¹ | Ar² | Ar³ |
|---|---|---|---|
| 100001 | A1 | A1 | L1-B1 |
| 100002 | A1 | A1 | L1-B2 |
| 100003 | A1 | A1 | L1-B3 |
| 100004 | A1 | A1 | L1-B4 |
| 100005 | A1 | A1 | L1-B5 |
| 100006 | A1 | A1 | L1-B6 |
| 100007 | A1 | A1 | L1-B7 |
| 100008 | A1 | A1 | L1-B8 |
| 100009 | A1 | A1 | L1-B9 |
| 100010 | A1 | A1 | L1-B10 |
| 100011 | A1 | A1 | L1-B11 |
| 100012 | A1 | A1 | L1-B12 |
| 100013 | A1 | A1 | L1-B13 |
| 100014 | A1 | A1 | L1-B14 |
| 100015 | A1 | A1 | L2-B1 |
| 100016 | A1 | A1 | L2-B2 |
| 100017 | A1 | A1 | L2-B3 |
| 100018 | A1 | A1 | L2-B4 |
| 100019 | A1 | A1 | L2-B5 |
| 100020 | A1 | A1 | L2-B6 |
| 100021 | A1 | A1 | L2-B7 |
| 100022 | A1 | A1 | L2-B8 |
| 100023 | A1 | A1 | L2-B9 |
| 100024 | A1 | A1 | L2-B10 |
| 100025 | A1 | A1 | L2-B11 |
| 100026 | A1 | A1 | L2-B12 |
| 100027 | A1 | A1 | L2-B13 |
| 100028 | A1 | A1 | L2-B14 |
| 100029 | A1 | A1 | L3-B1 |
| 100030 | A1 | A1 | L3-B2 |
| 100031 | A1 | A1 | L3-B3 |
| 100032 | A1 | A1 | L3-B4 |
| 100033 | A1 | A1 | L3-B5 |
| 100034 | A1 | A1 | L3-B6 |
| 100035 | A1 | A1 | L3-B7 |
| 100036 | A1 | A1 | L3-B8 |
| 100037 | A1 | A1 | L3-B9 |
| 100038 | A1 | A1 | L3-B10 |
| 100039 | A1 | A1 | L3-B11 |
| 100040 | A1 | A1 | L3-B12 |
| 100041 | A1 | A1 | L3-B13 |
| 100042 | A1 | A1 | L3-B14 |
| 100043 | A1 | A1 | L4-B1 |
| 100044 | A1 | A1 | L4-B2 |
| 100045 | A1 | A1 | L4-B3 |
| 100046 | A1 | A1 | L4-B4 |
| 100047 | A1 | A1 | L4-B5 |
| 100048 | A1 | A1 | L4-B6 |
| 100049 | A1 | A1 | L4-B7 |
| 100050 | A1 | A1 | L4-B8 |
| 100051 | A1 | A1 | L4-B9 |
| 100052 | A1 | A1 | L4-B10 |
| 100053 | A1 | A1 | L4-B11 |
| 100054 | A1 | A1 | L4-B12 |
| 100055 | A1 | A1 | L4-B13 |
| 100056 | A1 | A1 | L4-B14 |
| 100057 | A1 | A1 | L5-B1 |
| 100058 | A1 | A1 | L5-B2 |
| 100059 | A1 | A1 | L5-B3 |
| 100060 | A1 | A1 | L5-B4 |
| 100061 | A1 | A1 | L5-B5 |
| 100062 | A1 | A1 | L5-B6 |
| 100063 | A1 | A1 | L5-B7 |
| 100064 | A1 | A1 | L5-B8 |
| 100065 | A1 | A1 | L5-B9 |
| 100066 | A1 | A1 | L5-B10 |
| 100067 | A1 | A1 | L5-B11 |
| 100068 | A1 | A1 | L5-B12 |
| 100069 | A1 | A1 | L5-B13 |
| 100070 | A1 | A1 | L5-B14 |
| 100071 | A1 | A1 | L6-B1 |
| 100072 | A1 | A1 | L6-B2 |
| 100073 | A1 | A1 | L6-B3 |
| 100074 | A1 | A1 | L6-B4 |
| 100075 | A1 | A1 | L6-B5 |
| 100076 | A1 | A1 | L6-B6 |
| 100077 | A1 | A1 | L6-B7 |
| 100078 | A1 | A1 | L6-B8 |
| 100079 | A1 | A1 | L6-B9 |
| 100080 | A1 | A1 | L6-B10 |
| 100081 | A1 | A1 | L6-B11 |
| 100082 | A1 | A1 | L6-B12 |
| 100083 | A1 | A1 | L6-B13 |
| 100084 | A1 | A1 | L6-B14 |
| 100085 | A1 | A1 | L7-B1 |
| 100086 | A1 | A1 | L7-B2 |
| 100087 | A1 | A1 | L7-B3 |
| 100088 | A1 | A1 | L7-B4 |
| 100089 | A1 | A1 | L7-B5 |
| 100090 | A1 | A1 | L7-B6 |
| 100091 | A1 | A1 | L7-B7 |
| 100092 | A1 | A1 | L7-B8 |
| 100093 | A1 | A1 | L7-B9 |
| 100094 | A1 | A1 | L7-B10 |
| 100095 | A1 | A1 | L7-B11 |
| 100096 | A1 | A1 | L7-B12 |
| 100097 | A1 | A1 | L7-B13 |
| 100098 | A1 | A1 | L7-B14 |
| 100099 | A1 | A1 | L8-B1 |
| 100100 | A1 | A1 | L8-B2 |
| 100101 | A1 | A1 | L8-B3 |
| 100102 | A1 | A1 | L8-B4 |
| 100103 | A1 | A1 | L8-B5 |
| 100104 | A1 | A1 | L8-B6 |
| 100105 | A1 | A1 | L8-B7 |
| 100106 | A1 | A1 | L8-B8 |
| 100107 | A1 | A1 | L8-B9 |
| 100108 | A1 | A1 | L8-B10 |
| 100109 | A1 | A1 | L8-B11 |
| 100110 | A1 | A1 | L8-B12 |
| 100111 | A1 | A1 | L8-B13 |
| 100112 | A1 | A1 | L8-B14 |
| 100113 | A1 | A1 | L9-B1 |
| 100114 | A1 | A1 | L9-B2 |
| 100115 | A1 | A1 | L9-B3 |
| 100116 | A1 | A1 | L9-B4 |
| 100117 | A1 | A1 | L9-B5 |
| 100118 | A1 | A1 | L9-B6 |
| 100119 | A1 | A1 | L9-B7 |
| 100120 | A1 | A1 | L9-B8 |
| 100121 | A1 | A1 | L9-B9 |
| 100122 | A1 | A1 | L9-B10 |
| 100123 | A1 | A1 | L9-B11 |
| 100124 | A1 | A1 | L9-B12 |
| 100125 | A1 | A1 | L9-B13 |
| 100126 | A1 | A1 | L9-B14 |
| 100127 | A1 | A1 | L10-B1 |
| 100128 | A1 | A1 | L10-B2 |
| 100129 | A1 | A1 | L10-B3 |
| 100130 | A1 | A1 | L10-B4 |
| 100131 | A1 | A1 | L10-B5 |
| 100132 | A1 | A1 | L10-B6 |
| 100133 | A1 | A1 | L10-B7 |
| 100134 | A1 | A1 | L10-B8 |
| 100135 | A1 | A1 | L10-B9 |
| 100136 | A1 | A1 | L10-B10 |
| 100137 | A1 | A1 | L10-B11 |
| 100138 | A1 | A1 | L10-B12 |
| 100139 | A1 | A1 | L10-B13 |
| 100140 | A1 | A1 | L10-B14 |
| 100141 | A1 | A1 | L11-B1 |
| 100142 | A1 | A1 | L11-B2 |
| 100143 | A1 | A1 | L11-B3 |
| 100144 | A1 | A1 | L11-B4 |
| 100145 | A1 | A1 | L11-B5 |
| 100146 | A1 | A1 | L11-B6 |
| 100147 | A1 | A1 | L11-B7 |
| 100148 | A1 | A1 | L11-B8 |
| 100149 | A1 | A1 | L11-B9 |
| 100150 | A1 | A1 | L11-B10 |
| 100151 | A1 | A1 | L11-B11 |
| 100152 | A1 | A1 | L11-B12 |
| 100153 | A1 | A1 | L11-B13 |
| 100154 | A1 | A1 | L11-B14 |
| 100155 | A1 | A1 | L12-B1 |
| 100156 | A1 | A1 | L12-B2 |
| 100157 | A1 | A1 | L12-B3 |
| 100158 | A1 | A1 | L12-B4 |
| 100159 | A1 | A1 | L12-B5 |
| 100160 | A1 | A1 | L12-B6 |
| 100161 | A1 | A1 | L12-B7 |
| 100162 | A1 | A1 | L12-B8 |
| 100163 | A1 | A1 | L12-B9 |
| 100164 | A1 | A1 | L12-B10 |
| 100165 | A1 | A1 | L12-B11 |
| 100166 | A1 | A1 | L12-B12 |
| 100167 | A1 | A1 | L12-B13 |
| 100168 | A1 | A1 | L12-B14 |
| 100169 | A1 | A1 | L13-B1 |
| 100170 | A1 | A1 | L13-B2 |
| 100171 | A1 | A1 | L13-B3 |
| 100172 | A1 | A1 | L13-B4 |
| 100173 | A1 | A1 | L13-B5 |
| 100174 | A1 | A1 | L13-B6 |
| 100175 | A1 | A1 | L13-B7 |
| 100176 | A1 | A1 | L13-B8 |
| 100177 | A1 | A1 | L13-B9 |
| 100178 | A1 | A1 | L13-B10 |
| 100179 | A1 | A1 | L13-B11 |
| 100180 | A1 | A1 | L13-B12 |
| 100181 | A1 | A1 | L13-B13 |
| 100182 | A1 | A1 | L13-B14 |
| 100183 | A1 | A1 | L14-B1 |
| 100184 | A1 | A1 | L14-B2 |
| 100185 | A1 | A1 | L14-B3 |
| 100186 | A1 | A1 | L14-B4 |
| 100187 | A1 | A1 | L14-B5 |
| 100188 | A1 | A1 | L14-B6 |
| 100189 | A1 | A1 | L14-B7 |
| 100190 | A1 | A1 | L14-B8 |
| 100191 | A1 | A1 | L14-B9 |
| 100192 | A1 | A1 | L14-B10 |
| 100193 | A1 | A1 | L14-B11 |
| 100194 | A1 | A1 | L14-B12 |
| 100195 | A1 | A1 | L14-B13 |
| 100196 | A1 | A1 | L14-B14 |
| 100197 | A1 | A1 | L15-B1 |
| 100198 | A1 | A1 | L15-B2 |
| 100199 | A1 | A1 | L15-B3 |
| 100200 | A1 | A1 | L15-B4 |
| 100201 | A1 | A1 | L15-B5 |
| 100202 | A1 | A1 | L15-B6 |
| 100203 | A1 | A1 | L15-B7 |
| 100204 | A1 | A1 | L15-B8 |
| 100205 | A1 | A1 | L15-B9 |
| 100206 | A1 | A1 | L15-B10 |
| 100207 | A1 | A1 | L15-B11 |
| 100208 | A1 | A1 | L15-B12 |
| 100209 | A1 | A1 | L15-B13 |
| 100210 | A1 | A1 | L15-B14 |
| 100211 | A1 | L1-B1 | L1-B1 |
| 100212 | A1 | L1-B2 | L1-B2 |
| 100213 | A1 | L1-B3 | L1-B3 |
| 100214 | A1 | L1-B4 | L1-B4 |
| 100215 | A1 | L1-B5 | L1-B5 |
| 100216 | A1 | L1-B6 | L1-B6 |
| 100217 | A1 | L1-B7 | L1-B7 |
| 100218 | A1 | L1-B8 | L1-B8 |
| 100219 | A1 | L1-B9 | L1-B9 |
| 100220 | A1 | L1-B10 | L1-B10 |
| 100221 | A1 | L1-B11 | L1-B11 |
| 100222 | A1 | L1-B12 | L1-B12 |
| 100223 | A1 | L1-B13 | L1-B13 |
| 100224 | A1 | L1-B14 | L1-B14 |
| 100225 | A1 | L2-B1 | L2-B1 |
| 100226 | A1 | L2-B2 | L2-B2 |
| 100227 | A1 | L2-B3 | L2-B3 |
| 100228 | A1 | L2-B4 | L2-B4 |
| 100229 | A1 | L2-B5 | L2-B5 |
| 100230 | A1 | L2-B6 | L2-B6 |
| 100231 | A1 | L2-B7 | L2-B7 |
| 100232 | A1 | L2-B8 | L2-B8 |
| 100233 | A1 | L2-B9 | L2-B9 |
| 100234 | A1 | L2-B10 | L2-B10 |
| 100235 | A1 | L2-B11 | L2-B11 |
| 100236 | A1 | L2-B12 | L2-B12 |
| 100237 | A1 | L2-B13 | L2-B13 |
| 100238 | A1 | L2-B14 | L2-B14 |
| 100239 | A1 | L3-B1 | L3-B1 |
| 100240 | A1 | L3-B2 | L3-B2 |
| 100241 | A1 | L3-B3 | L3-B3 |
| 100242 | A1 | L3-B4 | L3-B4 |
| 100243 | A1 | L3-B5 | L3-B5 |
| 100244 | A1 | L3-B6 | L3-B6 |
| 100245 | A1 | L3-B7 | L3-B7 |
| 100246 | A1 | L3-B8 | L3-B8 |
| 100247 | A1 | L3-B9 | L3-B9 |
| 100248 | A1 | L3-B10 | L3-B10 |
| 100249 | A1 | L3-B11 | L3-B11 |
| 100250 | A1 | L3-B12 | L3-B12 |
| 100251 | A1 | L3-B13 | L3-B13 |
| 100252 | A1 | L3-B14 | L3-B14 |
| 100253 | A1 | L4-B1 | L4-B1 |
| 100254 | A1 | L4-B2 | L4-B2 |
| 100255 | A1 | L4-B3 | L4-B3 |
| 100256 | A1 | L4-B4 | L4-B4 |
| 100257 | A1 | L4-B5 | L4-B5 |
| 100258 | A1 | L4-B6 | L4-B6 |
| 100259 | A1 | L4-B7 | L4-B7 |
| 100260 | A1 | L4-B8 | L4-B8 |
| 100261 | A1 | L4-B9 | L4-B9 |
| 100262 | A1 | L4-B10 | L4-B10 |
| 100263 | A1 | L4-B11 | L4-B11 |
| 100264 | A1 | L4-B12 | L4-B12 |
| 100265 | A1 | L4-B13 | L4-B13 |
| 100266 | A1 | L4-B14 | L4-B14 |
| 100267 | A1 | L5-B1 | L5-B1 |
| 100268 | A1 | L5-B2 | L5-B2 |
| 100269 | A1 | L5-B3 | L5-B3 |
| 100270 | A1 | L5-B4 | L5-B4 |
| 100271 | A1 | L5-B5 | L5-B5 |
| 100272 | A1 | L5-B6 | L5-B6 |
| 100273 | A1 | L5-B7 | L5-B7 |
| 100274 | A1 | L5-B8 | L5-B8 |
| 100275 | A1 | L5-B9 | L5-B9 |
| 100276 | A1 | L5-B10 | L5-B10 |
| 100277 | A1 | L5-B11 | L5-B11 |
| 100278 | A1 | L5-B12 | L5-B12 |
| 100279 | A1 | L5-B13 | L5-B13 |
| 100280 | A1 | L5-B14 | L5-B14 |
| 100281 | A1 | L6-B1 | L6-B1 |
| 100282 | A1 | L6-B2 | L6-B2 |
| 100283 | A1 | L6-B3 | L6-B3 |
| 100284 | A1 | L6-B4 | L6-B4 |
| 100285 | A1 | L6-B5 | L6-B5 |
| 100286 | A1 | L6-B6 | L6-B6 |
| 100287 | A1 | L6-B7 | L6-B7 |
| 100288 | A1 | L6-B8 | L6-B8 |
| 100289 | A1 | L6-B9 | L6-B9 |
| 100290 | A1 | L6-B10 | L6-B10 |
| 100291 | A1 | L6-B11 | L6-B11 |
| 100292 | A1 | L6-B12 | L6-B12 |
| 100293 | A1 | L6-B13 | L6-B13 |
| 100294 | A1 | L6-B14 | L6-B14 |
| 100295 | A1 | L7-B1 | L7-B1 |
| 100296 | A1 | L7-B2 | L7-B2 |
| 100297 | A1 | L7-B3 | L7-B3 |
| 100298 | A1 | L7-B4 | L7-B4 |
| 100299 | A1 | L7-B5 | L7-B5 |
| 100300 | A1 | L7-B6 | L7-B6 |
| 100301 | A1 | L7-B7 | L7-B7 |
| 100302 | A1 | L7-B8 | L7-B8 |
| 100303 | A1 | L7-B9 | L7-B9 |
| 100304 | A1 | L7-B10 | L7-B10 |
| 100305 | A1 | L7-B11 | L7-B11 |
| 100306 | A1 | L7-B12 | L7-B12 |
| 100307 | A1 | L7-B13 | L7-B13 |
| 100308 | A1 | L7-B14 | L7-B14 |
| 100309 | A1 | L8-B1 | L8-B1 |
| 100310 | A1 | L8-B2 | L8-B2 |
| 100311 | A1 | L8-B3 | L8-B3 |
| 100312 | A1 | L8-B4 | L8-B4 |
| 100313 | A1 | L8-B5 | L8-B5 |
| 100314 | A1 | L8-B6 | L8-B6 |
| 100315 | A1 | L8-B7 | L8-B7 |
| 100316 | A1 | L8-B8 | L8-B8 |
| 100317 | A1 | L8-B9 | L8-B9 |
| 100318 | A1 | L8-B10 | L8-B10 |
| 100319 | A1 | L8-B11 | L8-B11 |
| 100320 | A1 | L8-B12 | L8-B12 |
| 100321 | A1 | L8-B13 | L8-B13 |
| 100322 | A1 | L8-B14 | L8-B14 |
| 100323 | A1 | L9-B1 | L9-B1 |
| 100324 | A1 | L9-B2 | L9-B2 |
| 100325 | A1 | L9-B3 | L9-B3 |
| 100326 | A1 | L9-B4 | L9-B4 |
| 100327 | A1 | L9-B5 | L9-B5 |
| 100328 | A1 | L9-B6 | L9-B6 |
| 100329 | A1 | L9-B7 | L9-B7 |
| 100330 | A1 | L9-B8 | L9-B8 |
| 100331 | A1 | L9-B9 | L9-B9 |
| 100332 | A1 | L9-B10 | L9-B10 |
| 100333 | A1 | L9-B11 | L9-B11 |
| 100334 | A1 | L9-B12 | L9-B12 |
| 100335 | A1 | L9-B13 | L9-B13 |
| 100336 | A1 | L9-B14 | L9-B14 |
| 100337 | A1 | L10-B1 | L10-B1 |
| 100338 | A1 | L10-B2 | L10-B2 |
| 100339 | A1 | L10-B3 | L10-B3 |
| 100340 | A1 | L10-B4 | L10-B4 |
| 100341 | A1 | L10-B5 | L10-B5 |
| 100342 | A1 | L10-B6 | L10-B6 |
| 100343 | A1 | L10-B7 | L10-B7 |
| 100344 | A1 | L10-B8 | L10-B8 |
| 100345 | A1 | L10-B9 | L10-B9 |
| 100346 | A1 | L10-B10 | L10-B10 |
| 100347 | A1 | L10-B11 | L10-B11 |
| 100348 | A1 | L10-B12 | L10-B12 |
| 100349 | A1 | L10-B13 | L10-B13 |
| 100350 | A1 | L10-B14 | L10-B14 |
| 100351 | A1 | L11-B1 | L11-B1 |
| 100352 | A1 | L11-B2 | L11-B2 |
| 100353 | A1 | L11-B3 | L11-B3 |
| 100354 | A1 | L11-B4 | L11-B4 |
| 100355 | A1 | L11-B5 | L11-B5 |
| 100356 | A1 | L11-B6 | L11-B6 |
| 100357 | A1 | L11-B7 | L11-B7 |
| 100358 | A1 | L11-B8 | L11-B8 |
| 100359 | A1 | L11-B9 | L11-B9 |
| 100360 | A1 | L11-B10 | L11-B10 |
| 100361 | A1 | L11-B11 | L11-B11 |
| 100362 | A1 | L11-B12 | L11-B12 |
| 100363 | A1 | L11-B13 | L11-B13 |
| 100364 | A1 | L11-B14 | L11-B14 |
| 100365 | A1 | L12-B1 | L12-B1 |
| 100366 | A1 | L12-B2 | L12-B2 |
| 100367 | A1 | L12-B3 | L12-B3 |
| 100368 | A1 | L12-B4 | L12-B4 |
| 100369 | A1 | L12-B5 | L12-B5 |
| 100370 | A1 | L12-B6 | L12-B6 |
| 100371 | A1 | L12-B7 | L12-B7 |
| 100372 | A1 | L12-B8 | L12-B8 |
| 100373 | A1 | L12-B9 | L12-B9 |
| 100374 | A1 | L12-B10 | L12-B10 |
| 100375 | A1 | L12-B11 | L12-B11 |
| 100376 | A1 | L12-B12 | L12-B12 |
| 100377 | A1 | L12-B13 | L12-B13 |
| 100378 | A1 | L12-B14 | L12-B14 |
| 100379 | A1 | L13-B1 | L13-B1 |
| 100380 | A1 | L13-B2 | L13-B2 |
| 100381 | A1 | L13-B3 | L13-B3 |
| 100382 | A1 | L13-B4 | L13-B4 |
| 100383 | A1 | L13-B5 | L13-B5 |
| 100384 | A1 | L13-B6 | L13-B6 |
| 100385 | A1 | L13-B7 | L13-B7 |
| 100386 | A1 | L13-B8 | L13-B8 |
| 100387 | A1 | L13-B9 | L13-B9 |
| 100388 | A1 | L13-B10 | L13-B10 |
| 100389 | A1 | L13-B11 | L13-B11 |
| 100390 | A1 | L13-B12 | L13-B12 |
| 100391 | A1 | L13-B13 | L13-B13 |
| 100392 | A1 | L13-B14 | L13-B14 |
| 100393 | A1 | L14-B1 | L14-B1 |
| 100394 | A1 | L14-B2 | L14-B2 |
| 100395 | A1 | L14-B3 | L14-B3 |
| 100396 | A1 | L14-B4 | L14-B4 |
| 100397 | A1 | L14-B5 | L14-B5 |
| 100398 | A1 | L14-B6 | L14-B6 |
| 100399 | A1 | L14-B7 | L14-B7 |
| 100400 | A1 | L14-B8 | L14-B8 |
| 100401 | A1 | L14-B9 | L14-B9 |
| 100402 | A1 | L14-B10 | L14-B10 |
| 100403 | A1 | L14-B11 | L14-B11 |
| 100404 | A1 | L14-B12 | L14-B12 |
| 100405 | A1 | L14-B13 | L14-B13 |
| 100406 | A1 | L14-B14 | L14-B14 |
| 100407 | A1 | L15-B1 | L15-B1 |
| 100408 | A1 | L15-B2 | L15-B2 |
| 100409 | A1 | L15-B3 | L15-B3 |
| 100410 | A1 | L15-B4 | L15-B4 |
| 100411 | A1 | L15-B5 | L15-B5 |
| 100412 | A1 | L15-B6 | L15-B6 |
| 100413 | A1 | L15-B7 | L15-B7 |
| 100414 | A1 | L15-B8 | L15-B8 |
| 100415 | A1 | L15-B9 | L15-B9 |
| 100416 | A1 | L15-B10 | L15-B10 |
| 100417 | A1 | L15-B11 | L15-B11 |
| 100418 | A1 | L15-B12 | L15-B12 |
| 100419 | A1 | L15-B13 | L15-B13 |
| 100420 | A1 | L15-B14 | L15-B14 |
| 100421 | A2 | A2 | L1-B1 |
| 100422 | A2 | A2 | L1-B2 |
| 100423 | A2 | A2 | L1-B3 |
| 100424 | A2 | A2 | L1-B4 |
| 100425 | A2 | A2 | L1-B5 |
| 100426 | A2 | A2 | L1-B6 |
| 100427 | A2 | A2 | L1-B7 |
| 100428 | A2 | A2 | L1-B8 |
| 100429 | A2 | A2 | L1-B9 |
| 100430 | A2 | A2 | L1-B10 |
| 100431 | A2 | A2 | L1-B11 |
| 100432 | A2 | A2 | L1-B12 |
| 100433 | A2 | A2 | L1-B13 |
| 100434 | A2 | A2 | L1-B14 |
| 100435 | A2 | A2 | L2-B1 |
| 100436 | A2 | A2 | L2-B2 |
| 100437 | A2 | A2 | L2-B3 |
| 100438 | A2 | A2 | L2-B4 |
| 100439 | A2 | A2 | L2-B5 |
| 100440 | A2 | A2 | L2-B6 |
| 100441 | A2 | A2 | L2-B7 |
| 100442 | A2 | A2 | L2-B8 |
| 100443 | A2 | A2 | L2-B9 |
| 100444 | A2 | A2 | L2-B10 |
| 100445 | A2 | A2 | L2-B11 |
| 100446 | A2 | A2 | L2-B12 |
| 100447 | A2 | A2 | L2-B13 |
| 100448 | A2 | A2 | L2-B14 |
| 100449 | A2 | A2 | L3-B1 |
| 100450 | A2 | A2 | L3-B2 |
| 100451 | A2 | A2 | L3-B3 |
| 100452 | A2 | A2 | L3-B4 |
| 100453 | A2 | A2 | L3-B5 |
| 100454 | A2 | A2 | L3-B6 |
| 100455 | A2 | A2 | L3-B7 |
| 100456 | A2 | A2 | L3-B8 |
| 100457 | A2 | A2 | L3-B9 |
| 100458 | A2 | A2 | L3-B10 |
| 100459 | A2 | A2 | L3-B11 |
| 100460 | A2 | A2 | L3-B12 |
| 100461 | A2 | A2 | L3-B13 |
| 100462 | A2 | A2 | L3-B14 |
| 100463 | A2 | A2 | L4-B1 |
| 100464 | A2 | A2 | L4-B2 |
| 100465 | A2 | A2 | L4-B3 |
| 100466 | A2 | A2 | L4-B4 |
| 100467 | A2 | A2 | L4-B5 |
| 100468 | A2 | A2 | L4-B6 |
| 100469 | A2 | A2 | L4-B7 |
| 100470 | A2 | A2 | L4-B8 |
| 100471 | A2 | A2 | L4-B9 |
| 100472 | A2 | A2 | L4-B10 |
| 100473 | A2 | A2 | L4-B11 |
| 100474 | A2 | A2 | L4-B12 |
| 100475 | A2 | A2 | L4-B13 |
| 100476 | A2 | A2 | L4-B14 |
| 100477 | A2 | A2 | L5-B1 |
| 100478 | A2 | A2 | L5-B2 |
| 100479 | A2 | A2 | L5-B3 |
| 100480 | A2 | A2 | L5-B4 |
| 100481 | A2 | A2 | L5-B5 |
| 100482 | A2 | A2 | L5-B6 |
| 100483 | A2 | A2 | L5-B7 |
| 100484 | A2 | A2 | L5-B8 |
| 100485 | A2 | A2 | L5-B9 |
| 100486 | A2 | A2 | L5-B10 |
| 100487 | A2 | A2 | L5-B11 |
| 100488 | A2 | A2 | L5-B12 |
| 100489 | A2 | A2 | L5-B13 |
| 100490 | A2 | A2 | L5-B14 |
| 100491 | A2 | A2 | L6-B1 |
| 100492 | A2 | A2 | L6-B2 |
| 100493 | A2 | A2 | L6-B3 |
| 100494 | A2 | A2 | L6-B4 |
| 100495 | A2 | A2 | L6-B5 |
| 100496 | A2 | A2 | L6-B6 |
| 100497 | A2 | A2 | L6-B7 |
| 100498 | A2 | A2 | L6-B8 |
| 100499 | A2 | A2 | L6-B9 |
| 100500 | A2 | A2 | L6-B10 |
| 100501 | A2 | A2 | L6-B11 |
| 100502 | A2 | A2 | L6-B12 |
| 100503 | A2 | A2 | L6-B13 |
| 100504 | A2 | A2 | L6-B14 |
| 100505 | A2 | A2 | L7-B1 |
| 100506 | A2 | A2 | L7-B2 |
| 100507 | A2 | A2 | L7-B3 |
| 100508 | A2 | A2 | L7-B4 |
| 100509 | A2 | A2 | L7-B5 |
| 100510 | A2 | A2 | L7-B6 |
| 100511 | A2 | A2 | L7-B7 |
| 100512 | A2 | A2 | L7-B8 |
| 100513 | A2 | A2 | L7-B9 |
| 100514 | A2 | A2 | L7-B10 |
| 100515 | A2 | A2 | L7-B11 |
| 100516 | A2 | A2 | L7-B12 |
| 100517 | A2 | A2 | L7-B13 |
| 100518 | A2 | A2 | L7-B14 |
| 100519 | A2 | A2 | L8-B1 |
| 100520 | A2 | A2 | L8-B2 |
| 100521 | A2 | A2 | L8-B3 |
| 100522 | A2 | A2 | L8-B4 |
| 100523 | A2 | A2 | L8-B5 |
| 100524 | A2 | A2 | L8-B6 |
| 100525 | A2 | A2 | L8-B7 |
| 100526 | A2 | A2 | L8-B8 |
| 100527 | A2 | A2 | L8-B9 |
| 100528 | A2 | A2 | L8-B10 |
| 100529 | A2 | A2 | L8-B11 |
| 100530 | A2 | A2 | L8-B12 |
| 100531 | A2 | A2 | L8-B13 |
| 100532 | A2 | A2 | L8-B14 |
| 100533 | A2 | A2 | L9-B1 |
| 100534 | A2 | A2 | L9-B2 |
| 100535 | A2 | A2 | L9-B3 |
| 100536 | A2 | A2 | L9-B4 |
| 100537 | A2 | A2 | L9-B5 |
| 100538 | A2 | A2 | L9-B6 |
| 100539 | A2 | A2 | L9-B7 |
| 100540 | A2 | A2 | L9-B8 |
| 100541 | A2 | A2 | L9-B9 |
| 100542 | A2 | A2 | L9-B10 |
| 100543 | A2 | A2 | L9-B11 |
| 100544 | A2 | A2 | L9-B12 |
| 100545 | A2 | A2 | L9-B13 |
| 100546 | A2 | A2 | L9-B14 |
| 100547 | A2 | A2 | L10-B1 |
| 100548 | A2 | A2 | L10-B2 |
| 100549 | A2 | A2 | L10-B3 |
| 100550 | A2 | A2 | L10-B4 |
| 100551 | A2 | A2 | L10-B5 |
| 100552 | A2 | A2 | L10-B6 |
| 100553 | A2 | A2 | L10-B7 |
| 100554 | A2 | A2 | L10-B8 |
| 100555 | A2 | A2 | L10-B9 |
| 100556 | A2 | A2 | L10-B10 |
| 100557 | A2 | A2 | L10-B11 |
| 100558 | A2 | A2 | L10-B12 |
| 100559 | A2 | A2 | L10-B13 |
| 100560 | A2 | A2 | L10-B14 |
| 100561 | A2 | A2 | L11-B1 |
| 100562 | A2 | A2 | L11-B2 |
| 100563 | A2 | A2 | L11-B3 |
| 100564 | A2 | A2 | L11-B4 |
| 100565 | A2 | A2 | L11-B5 |
| 100566 | A2 | A2 | L11-B6 |
| 100567 | A2 | A2 | L11-B7 |
| 100568 | A2 | A2 | L11-B8 |
| 100569 | A2 | A2 | L11-B9 |
| 100570 | A2 | A2 | L11-B10 |
| 100571 | A2 | A2 | L11-B11 |
| 100572 | A2 | A2 | L11-B12 |
| 100573 | A2 | A2 | L11-B13 |
| 100574 | A2 | A2 | L11-B14 |
| 100575 | A2 | A2 | L12-B1 |
| 100576 | A2 | A2 | L12-B2 |
| 100577 | A2 | A2 | L12-B3 |
| 100578 | A2 | A2 | L12-B4 |
| 100579 | A2 | A2 | L12-B5 |
| 100580 | A2 | A2 | L12-B6 |
| 100581 | A2 | A2 | L12-B7 |
| 100582 | A2 | A2 | L12-B8 |
| 100583 | A2 | A2 | L12-B9 |
| 100584 | A2 | A2 | L12-B10 |
| 100585 | A2 | A2 | L12-B11 |
| 100586 | A2 | A2 | L12-B12 |
| 100587 | A2 | A2 | L12-B13 |
| 100588 | A2 | A2 | L12-B14 |
| 100589 | A2 | A2 | L13-B1 |
| 100590 | A2 | A2 | L13-B2 |
| 100591 | A2 | A2 | L13-B3 |
| 100592 | A2 | A2 | L13-B4 |
| 100593 | A2 | A2 | L13-B5 |
| 100594 | A2 | A2 | L13-B6 |
| 100595 | A2 | A2 | L13-B7 |
| 100596 | A2 | A2 | L13-B8 |
| 100597 | A2 | A2 | L13-B9 |
| 100598 | A2 | A2 | L13-B10 |
| 100599 | A2 | A2 | L13-B11 |
| 100600 | A2 | A2 | L13-B12 |
| 100601 | A2 | A2 | L13-B13 |
| 100602 | A2 | A2 | L13-B14 |
| 100603 | A2 | A2 | L14-B1 |
| 100604 | A2 | A2 | L14-B2 |
| 100605 | A2 | A2 | L14-B3 |
| 100606 | A2 | A2 | L14-B4 |
| 100607 | A2 | A2 | L14-B5 |
| 100608 | A2 | A2 | L14-B6 |
| 100609 | A2 | A2 | L14-B7 |
| 100610 | A2 | A2 | L14-B8 |
| 100611 | A2 | A2 | L14-B9 |
| 100612 | A2 | A2 | L14-B10 |
| 100613 | A2 | A2 | L14-B11 |
| 100614 | A2 | A2 | L14-B12 |
| 100615 | A2 | A2 | L14-B13 |
| 100616 | A2 | A2 | L14-B14 |
| 100617 | A2 | A2 | L15-B1 |
| 100618 | A2 | A2 | L15-B2 |
| 100619 | A2 | A2 | L15-B3 |
| 100620 | A2 | A2 | L15-B4 |
| 100621 | A2 | A2 | L15-B5 |
| 100622 | A2 | A2 | L15-B6 |
| 100623 | A2 | A2 | L15-B7 |
| 100624 | A2 | A2 | L15-B8 |
| 100625 | A2 | A2 | L15-B9 |
| 100626 | A2 | A2 | L15-B10 |
| 100627 | A2 | A2 | L15-B11 |
| 100628 | A2 | A2 | L15-B12 |
| 100629 | A2 | A2 | L15-B13 |
| 100630 | A2 | A2 | L15-B14 |
| 100631 | A2 | L1-B1 | L1-B1 |
| 100632 | A2 | L1-B2 | L1-B2 |
| 100633 | A2 | L1-B3 | L1-B3 |
| 100634 | A2 | L1-B4 | L1-B4 |
| 100635 | A2 | L1-B5 | L1-B5 |
| 100636 | A2 | L1-B6 | L1-B6 |
| 100637 | A2 | L1-B7 | L1-B7 |
| 100638 | A2 | L1-B8 | L1-B8 |
| 100639 | A2 | L1-B9 | L1-B9 |
| 100640 | A2 | L1-B10 | L1-B10 |
| 100641 | A2 | L1-B11 | L1-B11 |
| 100642 | A2 | L1-B12 | L1-B12 |
| 100643 | A2 | L1-B13 | L1-B13 |
| 100644 | A2 | L1-B14 | L1-B14 |
| 100645 | A2 | L2-B1 | L2-B1 |
| 100646 | A2 | L2-B2 | L2-B2 |
| 100647 | A2 | L2-B3 | L2-B3 |
| 100648 | A2 | L2-B4 | L2-B4 |
| 100649 | A2 | L2-B5 | L2-B5 |
| 100650 | A2 | L2-B6 | L2-B6 |
| 100651 | A2 | L2-B7 | L2-B7 |
| 100652 | A2 | L2-B8 | L2-B8 |
| 100653 | A2 | L2-B9 | L2-B9 |
| 100654 | A2 | L2-B10 | L2-B10 |
| 100655 | A2 | L2-B11 | L2-B11 |
| 100656 | A2 | L2-B12 | L2-B12 |
| 100657 | A2 | L2-B13 | L2-B13 |
| 100658 | A2 | L2-B14 | L2-B14 |
| 100659 | A2 | L3-B1 | L3-B1 |
| 100660 | A2 | L3-B2 | L3-B2 |
| 100661 | A2 | L3-B3 | L3-B3 |
| 100662 | A2 | L3-B4 | L3-B4 |
| 100663 | A2 | L3-B5 | L3-B5 |
| 100664 | A2 | L3-B6 | L3-B6 |
| 100665 | A2 | L3-B7 | L3-B7 |
| 100666 | A2 | L3-B8 | L3-B8 |
| 100667 | A2 | L3-B9 | L3-B9 |
| 100668 | A2 | L3-B10 | L3-B10 |
| 100669 | A2 | L3-B11 | L3-B11 |
| 100670 | A2 | L3-B12 | L3-B12 |
| 100671 | A2 | L3-B13 | L3-B13 |
| 100672 | A2 | L3-B14 | L3-B14 |
| 100673 | A2 | L4-B1 | L4-B1 |
| 100674 | A2 | L4-B2 | L4-B2 |
| 100675 | A2 | L4-B3 | L4-B3 |
| 100676 | A2 | L4-B4 | L4-B4 |
| 100677 | A2 | L4-B5 | L4-B5 |
| 100678 | A2 | L4-B6 | L4-B6 |
| 100679 | A2 | L4-B7 | L4-B7 |
| 100680 | A2 | L4-B8 | L4-B8 |
| 100681 | A2 | L4-B9 | L4-B9 |
| 100682 | A2 | L4-B10 | L4-B10 |
| 100683 | A2 | L4-B11 | L4-B11 |
| 100684 | A2 | L4-B12 | L4-B12 |
| 100685 | A2 | L4-B13 | L4-B13 |
| 100686 | A2 | L4-B14 | L4-B14 |
| 100687 | A2 | L5-B1 | L5-B1 |
| 100688 | A2 | L5-B2 | L5-B2 |
| 100689 | A2 | L5-B3 | L5-B3 |
| 100690 | A2 | L5-B4 | L5-B4 |
| 100691 | A2 | L5-B5 | L5-B5 |
| 100692 | A2 | L5-B6 | L5-B6 |
| 100693 | A2 | L5-B7 | L5-B7 |
| 100694 | A2 | L5-B8 | L5-B8 |
| 100695 | A2 | L5-B9 | L5-B9 |
| 100696 | A2 | L5-B10 | L5-B10 |
| 100697 | A2 | L5-B11 | L5-B11 |
| 100698 | A2 | L5-B12 | L5-B12 |
| 100699 | A2 | L5-B13 | L5-B13 |
| 100700 | A2 | L5-B14 | L5-B14 |
| 100701 | A2 | L6-B1 | L6-B1 |
| 100702 | A2 | L6-B2 | L6-B2 |
| 100703 | A2 | L6-B3 | L6-B3 |
| 100704 | A2 | L6-B4 | L6-B4 |
| 100705 | A2 | L6-B5 | L6-B5 |
| 100706 | A2 | L6-B6 | L6-B6 |
| 100707 | A2 | L6-B7 | L6-B7 |
| 100708 | A2 | L6-B8 | L6-B8 |
| 100709 | A2 | L6-B9 | L6-B9 |
| 100710 | A2 | L6-B10 | L6-B10 |
| 100711 | A2 | L6-B11 | L6-B11 |
| 100712 | A2 | L6-B12 | L6-B12 |
| 100713 | A2 | L6-B13 | L6-B13 |
| 100714 | A2 | L6-B14 | L6-B14 |
| 100715 | A2 | L7-B1 | L7-B1 |
| 100716 | A2 | L7-B2 | L7-B2 |
| 100717 | A2 | L7-B3 | L7-B3 |
| 100718 | A2 | L7-B4 | L7-B4 |
| 100719 | A2 | L7-B5 | L7-B5 |
| 100720 | A2 | L7-B6 | L7-B6 |
| 100721 | A2 | L7-B7 | L7-B7 |
| 100722 | A2 | L7-B8 | L7-B8 |
| 100723 | A2 | L7-B9 | L7-B9 |
| 100724 | A2 | L7-B10 | L7-B10 |
| 100725 | A2 | L7-B11 | L7-B11 |
| 100726 | A2 | L7-B12 | L7-B12 |
| 100727 | A2 | L7-B13 | L7-B13 |
| 100728 | A2 | L7-B14 | L7-B14 |
| 100729 | A2 | L8-B1 | L8-B1 |
| 100730 | A2 | L8-B2 | L8-B2 |
| 100731 | A2 | L8-B3 | L8-B3 |
| 100732 | A2 | L8-B4 | L8-B4 |
| 100733 | A2 | L8-B5 | L8-B5 |
| 100734 | A2 | L8-B6 | L8-B6 |
| 100735 | A2 | L8-B7 | L8-B7 |
| 100736 | A2 | L8-B8 | L8-B8 |
| 100737 | A2 | L8-B9 | L8-B9 |
| 100738 | A2 | L8-B10 | L8-B10 |
| 100739 | A2 | L8-B11 | L8-B11 |
| 100740 | A2 | L8-B12 | L8-B12 |
| 100741 | A2 | L8-B13 | L8-B13 |
| 100742 | A2 | L8-B14 | L8-B14 |
| 100743 | A2 | L9-B1 | L9-B1 |
| 100744 | A2 | L9-B2 | L9-B2 |
| 100745 | A2 | L9-B3 | L9-B3 |
| 100746 | A2 | L9-B4 | L9-B4 |
| 100747 | A2 | L9-B5 | L9-B5 |
| 100748 | A2 | L9-B6 | L9-B6 |
| 100749 | A2 | L9-B7 | L9-B7 |
| 100750 | A2 | L9-B8 | L9-B8 |
| 100751 | A2 | L9-B9 | L9-B9 |
| 100752 | A2 | L9-B10 | L9-B10 |
| 100753 | A2 | L9-B11 | L9-B11 |
| 100754 | A2 | L9-B12 | L9-B12 |
| 100755 | A2 | L9-B13 | L9-B13 |
| 100756 | A2 | L9-B14 | L9-B14 |
| 100757 | A2 | L10-B1 | L10-B1 |
| 100758 | A2 | L10-B2 | L10-B2 |
| 100759 | A2 | L10-B3 | L10-B3 |
| 100760 | A2 | L10-B4 | L10-B4 |
| 100761 | A2 | L10-B5 | L10-B5 |
| 100762 | A2 | L10-B6 | L10-B6 |
| 100763 | A2 | L10-B7 | L10-B7 |
| 100764 | A2 | L10-B8 | L10-B8 |
| 100765 | A2 | L10-B9 | L10-B9 |
| 100766 | A2 | L10-B10 | L10-B10 |
| 100767 | A2 | L10-B11 | L10-B11 |
| 100768 | A2 | L10-B12 | L10-B12 |
| 100769 | A2 | L10-B13 | L10-B13 |
| 100770 | A2 | L10-B14 | L10-B14 |
| 100771 | A2 | L11-B1 | L11-B1 |
| 100772 | A2 | L11-B2 | L11-B2 |
| 100773 | A2 | L11-B3 | L11-B3 |
| 100774 | A2 | L11-B4 | L11-B4 |
| 100775 | A2 | L11-B5 | L11-B5 |
| 100776 | A2 | L11-B6 | L11-B6 |
| 100777 | A2 | L11-B7 | L11-B7 |
| 100778 | A2 | L11-B8 | L11-B8 |
| 100779 | A2 | L11-B9 | L11-B9 |
| 100780 | A2 | L11-B10 | L11-B10 |
| 100781 | A2 | L11-B11 | L11-B11 |
| 100782 | A2 | L11-B12 | L11-B12 |
| 100783 | A2 | L11-B13 | L11-B13 |
| 100784 | A2 | L11-B14 | L11-B14 |
| 100785 | A2 | L12-B1 | L12-B1 |
| 100786 | A2 | L12-B2 | L12-B2 |
| 100787 | A2 | L12-B3 | L12-B3 |
| 100788 | A2 | L12-B4 | L12-B4 |
| 100789 | A2 | L12-B5 | L12-B5 |
| 100790 | A2 | L12-B6 | L12-B6 |
| 100791 | A2 | L12-B7 | L12-B7 |
| 100792 | A2 | L12-B8 | L12-B8 |
| 100793 | A2 | L12-B9 | L12-B9 |
| 100794 | A2 | L12-B10 | L12-B10 |
| 100795 | A2 | L12-B11 | L12-B11 |
| 100796 | A2 | L12-B12 | L12-B12 |
| 100797 | A2 | L12-B13 | L12-B13 |
| 100798 | A2 | L12-B14 | L12-B14 |
| 100799 | A2 | L13-B1 | L13-B1 |
| 100800 | A2 | L13-B2 | L13-B2 |
| 100801 | A2 | L13-B3 | L13-B3 |
| 100802 | A2 | L13-B4 | L13-B4 |
| 100803 | A2 | L13-B5 | L13-B5 |
| 100804 | A2 | L13-B6 | L13-B6 |
| 100805 | A2 | L13-B7 | L13-B7 |
| 100806 | A2 | L13-B8 | L13-B8 |
| 100807 | A2 | L13-B9 | L13-B9 |
| 100808 | A2 | L13-B10 | L13-B10 |
| 100809 | A2 | L13-B11 | L13-B11 |
| 100810 | A2 | L13-B12 | L13-B12 |
| 100811 | A2 | L13-B13 | L13-B13 |
| 100812 | A2 | L13-B14 | L13-B14 |
| 100813 | A2 | L14-B1 | L14-B1 |
| 100814 | A2 | L14-B2 | L14-B2 |
| 100815 | A2 | L14-B3 | L14-B3 |
| 100816 | A2 | L14-B4 | L14-B4 |
| 100817 | A2 | L14-B5 | L14-B5 |
| 100818 | A2 | L14-B6 | L14-B6 |
| 100819 | A2 | L14-B7 | L14-B7 |
| 100820 | A2 | L14-B8 | L14-B8 |
| 100821 | A2 | L14-B9 | L14-B9 |
| 100822 | A2 | L14-B10 | L14-B10 |
| 100823 | A2 | L14-B11 | L14-B11 |
| 100824 | A2 | L14-B12 | L14-B12 |
| 100825 | A2 | L14-B13 | L14-B13 |
| 100826 | A2 | L14-B14 | L14-B14 |
| 100827 | A2 | L15-B1 | L15-B1 |
| 100828 | A2 | L15-B2 | L15-B2 |
| 100829 | A2 | L15-B3 | L15-B3 |
| 100830 | A2 | L15-B4 | L15-B4 |
| 100831 | A2 | L15-B5 | L15-B5 |
| 100832 | A2 | L15-B6 | L15-B6 |
| 100833 | A2 | L15-B7 | L15-B7 |
| 100834 | A2 | L15-B8 | L15-B8 |
| 100835 | A2 | L15-B9 | L15-B9 |
| 100836 | A2 | L15-B10 | L15-B10 |
| 100837 | A2 | L15-B11 | L15-B11 |
| 100838 | A2 | L15-B12 | L15-B12 |
| 100839 | A2 | L15-B13 | L15-B13 |
| 100840 | A2 | L15-B14 | L15-B14 |
| 100841 | A3 | A3 | L1-B1 |
| 100842 | A3 | A3 | L1-B2 |
| 100843 | A3 | A3 | L1-B3 |
| 100844 | A3 | A3 | L1-B4 |
| 100845 | A3 | A3 | L1-B5 |
| 100846 | A3 | A3 | L1-B6 |
| 100847 | A3 | A3 | L1-B7 |
| 100848 | A3 | A3 | L1-B8 |
| 100849 | A3 | A3 | L1-B9 |
| 100850 | A3 | A3 | L1-B10 |
| 100851 | A3 | A3 | L1-B11 |
| 100852 | A3 | A3 | L1-B12 |
| 100853 | A3 | A3 | L1-B13 |
| 100854 | A3 | A3 | L1-B14 |
| 100855 | A3 | A3 | L2-B1 |
| 100856 | A3 | A3 | L2-B2 |
| 100857 | A3 | A3 | L2-B3 |
| 100858 | A3 | A3 | L2-B4 |
| 100859 | A3 | A3 | L2-B5 |
| 100860 | A3 | A3 | L2-B6 |
| 100861 | A3 | A3 | L2-B7 |
| 100862 | A3 | A3 | L2-B8 |
| 100863 | A3 | A3 | L2-B9 |
| 100864 | A3 | A3 | L2-B10 |
| 100865 | A3 | A3 | L2-B11 |
| 100866 | A3 | A3 | L2-B12 |
| 100867 | A3 | A3 | L2-B13 |
| 100868 | A3 | A3 | L2-B14 |
| 100869 | A3 | A3 | L3-B1 |
| 100870 | A3 | A3 | L3-B2 |
| 100871 | A3 | A3 | L3-B3 |
| 100872 | A3 | A3 | L3-B4 |
| 100873 | A3 | A3 | L3-B5 |
| 100874 | A3 | A3 | L3-B6 |
| 100875 | A3 | A3 | L3-B7 |
| 100876 | A3 | A3 | L3-B8 |
| 100877 | A3 | A3 | L3-B9 |
| 100878 | A3 | A3 | L3-B10 |
| 100879 | A3 | A3 | L3-B11 |
| 100880 | A3 | A3 | L3-B12 |
| 100881 | A3 | A3 | L3-B13 |
| 100882 | A3 | A3 | L3-B14 |
| 100883 | A3 | A3 | L4-B1 |
| 100884 | A3 | A3 | L4-B2 |
| 100885 | A3 | A3 | L4-B3 |
| 100886 | A3 | A3 | L4-B4 |
| 100887 | A3 | A3 | L4-B5 |
| 100888 | A3 | A3 | L4-B6 |
| 100889 | A3 | A3 | L4-B7 |
| 100890 | A3 | A3 | L4-B8 |
| 100891 | A3 | A3 | L4-B9 |
| 100892 | A3 | A3 | L4-B10 |
| 100893 | A3 | A3 | L4-B11 |
| 100894 | A3 | A3 | L4-B12 |
| 100895 | A3 | A3 | L4-B13 |
| 100896 | A3 | A3 | L4-B14 |
| 100897 | A3 | A3 | L5-B1 |
| 100898 | A3 | A3 | L5-B2 |
| 100899 | A3 | A3 | L5-B3 |
| 100900 | A3 | A3 | L5-B4 |
| 100901 | A3 | A3 | L5-B5 |
| 100902 | A3 | A3 | L5-B6 |
| 100903 | A3 | A3 | L5-B7 |
| 100904 | A3 | A3 | L5-B8 |
| 100905 | A3 | A3 | L5-B9 |
| 100906 | A3 | A3 | L5-B10 |
| 100907 | A3 | A3 | L5-B11 |
| 100908 | A3 | A3 | L5-B12 |
| 100909 | A3 | A3 | L5-B13 |
| 100910 | A3 | A3 | L5-B14 |
| 100911 | A3 | A3 | L6-B1 |
| 100912 | A3 | A3 | L6-B2 |
| 100913 | A3 | A3 | L6-B3 |
| 100914 | A3 | A3 | L6-B4 |
| 100915 | A3 | A3 | L6-B5 |
| 100916 | A3 | A3 | L6-B6 |
| 100917 | A3 | A3 | L6-B7 |
| 100918 | A3 | A3 | L6-B8 |
| 100919 | A3 | A3 | L6-B9 |
| 100920 | A3 | A3 | L6-B10 |
| 100921 | A3 | A3 | L6-B11 |
| 100922 | A3 | A3 | L6-B12 |
| 100923 | A3 | A3 | L6-B13 |
| 100924 | A3 | A3 | L6-B14 |
| 100925 | A3 | A3 | L7-B1 |
| 100926 | A3 | A3 | L7-B2 |
| 100927 | A3 | A3 | L7-B3 |
| 100928 | A3 | A3 | L7-B4 |
| 100929 | A3 | A3 | L7-B5 |
| 100930 | A3 | A3 | L7-B6 |
| 100931 | A3 | A3 | L7-B7 |
| 100932 | A3 | A3 | L7-B8 |
| 100933 | A3 | A3 | L7-B9 |
| 100934 | A3 | A3 | L7-B10 |
| 100935 | A3 | A3 | L7-B11 |
| 100936 | A3 | A3 | L7-B12 |
| 100937 | A3 | A3 | L7-B13 |
| 100938 | A3 | A3 | L7-B14 |
| 100939 | A3 | A3 | L8-B1 |
| 100940 | A3 | A3 | L8-B2 |
| 100941 | A3 | A3 | L8-B3 |
| 100942 | A3 | A3 | L8-B4 |
| 100943 | A3 | A3 | L8-B5 |
| 100944 | A3 | A3 | L8-B6 |
| 100945 | A3 | A3 | L8-B7 |
| 100946 | A3 | A3 | L8-B8 |
| 100947 | A3 | A3 | L8-B9 |
| 100948 | A3 | A3 | L8-B10 |
| 100949 | A3 | A3 | L8-B11 |
| 100950 | A3 | A3 | L8-B12 |
| 100951 | A3 | A3 | L8-B13 |
| 100952 | A3 | A3 | L8-B14 |
| 100953 | A3 | A3 | L9-B1 |
| 100954 | A3 | A3 | L9-B2 |
| 100955 | A3 | A3 | L9-B3 |
| 100956 | A3 | A3 | L9-B4 |
| 100957 | A3 | A3 | L9-B5 |
| 100958 | A3 | A3 | L9-B6 |
| 100959 | A3 | A3 | L9-B7 |
| 100960 | A3 | A3 | L9-B8 |
| 100961 | A3 | A3 | L9-B9 |
| 100962 | A3 | A3 | L9-B10 |
| 100963 | A3 | A3 | L9-B1 |
| 100964 | A3 | A3 | L9-B12 |
| 100965 | A3 | A3 | L9-B13 |
| 100966 | A3 | A3 | L9-B14 |
| 100967 | A3 | A3 | L10-B1 |
| 100968 | A3 | A3 | L10-B2 |
| 100969 | A3 | A3 | L10-B3 |
| 100970 | A3 | A3 | L10-B4 |
| 100971 | A3 | A3 | L10-B5 |
| 100972 | A3 | A3 | L10-B6 |
| 100973 | A3 | A3 | L10-B7 |
| 100974 | A3 | A3 | L10-B8 |
| 100975 | A3 | A3 | L10-B9 |
| 100976 | A3 | A3 | L10-B10 |
| 100977 | A3 | A3 | L10-B11 |
| 100978 | A3 | A3 | L10-B12 |
| 100979 | A3 | A3 | L10-B13 |
| 100980 | A3 | A3 | L10-B14 |
| 100981 | A3 | A3 | L11-B1 |
| 100982 | A3 | A3 | L11-B2 |
| 100983 | A3 | A3 | L11-B3 |
| 100984 | A3 | A3 | L11-B4 |
| 100985 | A3 | A3 | L11-B5 |
| 100986 | A3 | A3 | L11-B6 |
| 100987 | A3 | A3 | L11-B7 |
| 100988 | A3 | A3 | L11-B8 |
| 100989 | A3 | A3 | L11-B9 |
| 100990 | A3 | A3 | L11-B10 |
| 100991 | A3 | A3 | L11-B11 |
| 100992 | A3 | A3 | L11-B12 |
| 100993 | A3 | A3 | L11-B13 |
| 100994 | A3 | A3 | L11-B14 |
| 100995 | A3 | A3 | L12-B1 |
| 100996 | A3 | A3 | L12-B2 |
| 100997 | A3 | A3 | L12-B3 |
| 100998 | A3 | A3 | L12-B4 |
| 100999 | A3 | A3 | L12-B5 |
| 101000 | A3 | A3 | L12-B6 |
| 101001 | A3 | A3 | L12-B7 |
| 101002 | A3 | A3 | L12-B8 |
| 101003 | A3 | A3 | L12-B9 |
| 101004 | A3 | A3 | L12-B10 |
| 101005 | A3 | A3 | L12-B11 |
| 101006 | A3 | A3 | L12-B12 |
| 101007 | A3 | A3 | L12-B13 |
| 101008 | A3 | A3 | L12-B14 |
| 101009 | A3 | A3 | L13-B1 |
| 101010 | A3 | A3 | L13-B2 |
| 101011 | A3 | A3 | L13-B3 |
| 101012 | A3 | A3 | L13-B4 |
| 101013 | A3 | A3 | L13-B5 |
| 101014 | A3 | A3 | L13-B6 |
| 101015 | A3 | A3 | L13-B7 |
| 101016 | A3 | A3 | L13-B8 |
| 101017 | A3 | A3 | L13-B9 |
| 101018 | A3 | A3 | L13-B10 |
| 101019 | A3 | A3 | L13-B11 |
| 101020 | A3 | A3 | L13-B12 |
| 101021 | A3 | A3 | L13-B13 |
| 101022 | A3 | A3 | L13-B14 |
| 101023 | A3 | A3 | L14-B1 |
| 101024 | A3 | A3 | L14-B2 |
| 101025 | A3 | A3 | L14-B3 |
| 101026 | A3 | A3 | L14-B4 |
| 101027 | A3 | A3 | L14-B5 |
| 101028 | A3 | A3 | L14-B6 |
| 101029 | A3 | A3 | L14-B7 |
| 101030 | A3 | A3 | L14-B8 |
| 101031 | A3 | A3 | L14-B9 |
| 101032 | A3 | A3 | L14-B10 |
| 101033 | A3 | A3 | L14-B11 |
| 101034 | A3 | A3 | L14-B12 |
| 101035 | A3 | A3 | L14-B13 |
| 101036 | A3 | A3 | L14-B14 |
| 101037 | A3 | A3 | L15-B1 |
| 101038 | A3 | A3 | L15-B2 |
| 101039 | A3 | A3 | L15-B3 |
| 101040 | A3 | A3 | L15-B4 |
| 101041 | A3 | A3 | L15-B5 |
| 101042 | A3 | A3 | L15-B6 |
| 101043 | A3 | A3 | L15-B7 |
| 101044 | A3 | A3 | L15-B8 |
| 101045 | A3 | A3 | L15-B9 |
| 101046 | A3 | A3 | L15-B10 |
| 101047 | A3 | A3 | L15-B11 |
| 101048 | A3 | A3 | L15-B12 |
| 101049 | A3 | A3 | L15-B13 |
| 101050 | A3 | A3 | L15-B14 |
| 101051 | A3 | L1-B1 | L1-B1 |
| 101052 | A3 | L1-B2 | L1-B2 |
| 101053 | A3 | L1-B3 | L1-B3 |
| 101054 | A3 | L1-B4 | L1-B4 |
| 101055 | A3 | L1-B5 | L1-B5 |
| 101056 | A3 | L1-B6 | L1-B6 |
| 101057 | A3 | L1-B7 | L1-B7 |
| 101058 | A3 | L1-B8 | L1-B8 |
| 101059 | A3 | L1-B9 | L1-B9 |
| 101060 | A3 | L1-B10 | L1-B10 |
| 101061 | A3 | L1-B11 | L1-B11 |
| 101062 | A3 | L1-B12 | L1-B12 |
| 101063 | A3 | L1-B13 | L1-B13 |
| 101064 | A3 | L1-B14 | L1-B14 |
| 101065 | A3 | L2-B1 | L2-B1 |
| 101066 | A3 | L2-B2 | L2-B2 |
| 101067 | A3 | L2-B3 | L2-B3 |
| 101068 | A3 | L2-B4 | L2-B4 |
| 101069 | A3 | L2-B5 | L2-B5 |
| 101070 | A3 | L2-B6 | L2-B6 |
| 101071 | A3 | L2-B7 | L2-B7 |
| 101072 | A3 | L2-B8 | L2-B8 |
| 101073 | A3 | L2-B9 | L2-B9 |
| 101074 | A3 | L2-B10 | L2-B10 |
| 101075 | A3 | L2-B11 | L2-B11 |
| 101076 | A3 | L2-B12 | L2-B12 |
| 101077 | A3 | L2-B13 | L2-B13 |
| 101078 | A3 | L2-B14 | L2-B14 |
| 101079 | A3 | L3-B1 | L3-B1 |
| 101080 | A3 | L3-B2 | L3-B2 |
| 101081 | A3 | L3-B3 | L3-B3 |
| 101082 | A3 | L3-B4 | L3-B4 |
| 101083 | A3 | L3-B5 | L3-B5 |
| 101084 | A3 | L3-B6 | L3-B6 |
| 101085 | A3 | L3-B7 | L3-B7 |
| 101086 | A3 | L3-B8 | L3-B8 |
| 101087 | A3 | L3-B9 | L3-B9 |
| 101088 | A3 | L3-B10 | L3-B10 |
| 101089 | A3 | L3-B1 | L3-B11 |
| 101090 | A3 | L3-B12 | L3-B12 |
| 101091 | A3 | L3-B13 | L3-B13 |
| 101092 | A3 | L3-B14 | L3-B14 |
| 101093 | A3 | L4-B1 | L4-B1 |
| 101094 | A3 | L4-B2 | L4-B2 |
| 101095 | A3 | L4-B3 | L4-B3 |
| 101096 | A3 | L4-B4 | L4-B4 |
| 101097 | A3 | L4-B5 | L4-B5 |
| 101098 | A3 | L4-B6 | L4-B6 |
| 101099 | A3 | L4-B7 | L4-B7 |
| 101100 | A3 | L4-B8 | L4-B8 |
| 101101 | A3 | L4-B9 | L4-B9 |
| 101102 | A3 | L4-B10 | L4-B10 |
| 101103 | A3 | L4-B11 | L4-B11 |
| 101104 | A3 | L4-B12 | L4-B12 |
| 101105 | A3 | L4-B13 | L4-B13 |
| 101106 | A3 | L4-B14 | L4-B14 |
| 101107 | A3 | L5-B1 | L5-B1 |
| 101108 | A3 | L5-B2 | L5-B2 |
| 101109 | A3 | L5-B3 | L5-B3 |
| 101110 | A3 | L5-B4 | L5-B4 |
| 101111 | A3 | L5-B5 | L5-B5 |
| 101112 | A3 | L5-B6 | L5-B6 |
| 101113 | A3 | L5-B7 | L5-B7 |
| 101114 | A3 | L5-B8 | L5-B8 |
| 101115 | A3 | L5-B9 | L5-B9 |
| 101116 | A3 | L5-B10 | L5-B10 |
| 101117 | A3 | L5-B11 | L5-B11 |
| 101118 | A3 | L5-B12 | L5-B12 |
| 101119 | A3 | L5-B13 | L5-B13 |
| 101120 | A3 | L5-B14 | L5-B14 |
| 101121 | A3 | L6-B1 | L6-B1 |
| 101122 | A3 | L6-B2 | L6-B2 |
| 101123 | A3 | L6-B3 | L6-B3 |
| 101124 | A3 | L6-B4 | L6-B4 |
| 101125 | A3 | L6-B5 | L6-B5 |
| 101126 | A3 | L6-B6 | L6-B6 |
| 101127 | A3 | L6-B7 | L6-B7 |
| 101128 | A3 | L6-B8 | L6-B8 |
| 101129 | A3 | L6-B9 | L6-B9 |
| 101130 | A3 | L6-B10 | L6-B10 |
| 101131 | A3 | L6-B11 | L6-B11 |
| 101132 | A3 | L6-B12 | L6-B12 |
| 101133 | A3 | L6-B13 | L6-B13 |
| 101134 | A3 | L6-B14 | L6-B14 |
| 101135 | A3 | L7-B1 | L7-B1 |
| 101136 | A3 | L7-B2 | L7-B2 |
| 101137 | A3 | L7-B3 | L7-B3 |
| 101138 | A3 | L7-B4 | L7-B4 |
| 101139 | A3 | L7-B5 | L7-B5 |
| 101140 | A3 | L7-B6 | L7-B6 |
| 101141 | A3 | L7-B7 | L7-B7 |
| 101142 | A3 | L7-B8 | L7-B8 |
| 101143 | A3 | L7-B9 | L7-B9 |
| 101144 | A3 | L7-B10 | L7-B10 |
| 101145 | A3 | L7-B11 | L7-B11 |
| 101146 | A3 | L7-B12 | L7-B12 |
| 101147 | A3 | L7-B13 | L7-B13 |
| 101148 | A3 | L7-B14 | L7-B14 |
| 101149 | A3 | L8-B1 | L8-B1 |
| 101150 | A3 | L8-B2 | L8-B2 |
| 101151 | A3 | L8-B3 | L8-B3 |
| 101152 | A3 | L8-B4 | L8-B4 |
| 101153 | A3 | L8-B5 | L8-B5 |
| 101154 | A3 | L8-B6 | L8-B6 |
| 101155 | A3 | L8-B7 | L8-B7 |
| 101156 | A3 | L8-B8 | L8-B8 |
| 101157 | A3 | L8-B9 | L8-B9 |
| 101158 | A3 | L8-B10 | L8-B10 |
| 101159 | A3 | L8-B11 | L8-B11 |
| 101160 | A3 | L8-B12 | L8-B12 |
| 101161 | A3 | L8-B13 | L8-B13 |
| 101162 | A3 | L8-B14 | L8-B14 |
| 101163 | A3 | L9-B1 | L9-B1 |
| 101164 | A3 | L9-B2 | L9-B2 |
| 101165 | A3 | L9-B3 | L9-B3 |
| 101166 | A3 | L9-B4 | L9-B4 |
| 101167 | A3 | L9-B5 | L9-B5 |
| 101168 | A3 | L9-B6 | L9-B6 |
| 101169 | A3 | L9-B7 | L9-B7 |
| 101170 | A3 | L9-B8 | L9-B8 |
| 101171 | A3 | L9-B9 | L9-B9 |
| 101172 | A3 | L9-B10 | L9-B10 |
| 101173 | A3 | L9-B11 | L9-B11 |
| 101174 | A3 | L9-B12 | L9-B12 |
| 101175 | A3 | L9-B13 | L9-B13 |
| 101176 | A3 | L9-B14 | L9-B14 |
| 101177 | A3 | L10-B1 | L10-B1 |
| 101178 | A3 | L10-B2 | L10-B2 |
| 101179 | A3 | L10-B3 | L10-B3 |
| 101180 | A3 | L10-B4 | L10-B4 |
| 101181 | A3 | L10-B5 | L10-B5 |
| 101182 | A3 | L10-B6 | L10-B6 |
| 101183 | A3 | L10-B7 | L10-B7 |
| 101184 | A3 | L10-B8 | L10-B8 |
| 101185 | A3 | L10-B9 | L10-B9 |
| 101186 | A3 | L10-B10 | L10-B10 |
| 101187 | A3 | L10-B11 | L10-B11 |
| 101188 | A3 | L10-B12 | L10-B12 |
| 101189 | A3 | L10-B13 | L10-B13 |
| 101190 | A3 | L10-B14 | L10-B14 |
| 101191 | A3 | L11-B1 | L11-B1 |
| 101192 | A3 | L11-B2 | L11-B2 |
| 101193 | A3 | L11-B3 | L11-B3 |
| 101194 | A3 | L11-B4 | L11-B4 |
| 101195 | A3 | L11-B5 | L11-B5 |
| 101196 | A3 | L11-B6 | L11-B6 |
| 101197 | A3 | L11-B7 | L11-B7 |
| 101198 | A3 | L11-B8 | L11-B8 |
| 101199 | A3 | L11-B9 | L11-B9 |
| 101200 | A3 | L11-B10 | L11-B10 |
| 101201 | A3 | L11-B11 | L11-B11 |
| 101202 | A3 | L11-B12 | L11-B12 |
| 101203 | A3 | L11-B13 | L11-B13 |
| 101204 | A3 | L11-B14 | L11-B14 |
| 101205 | A3 | L12-B1 | L12-B1 |
| 101206 | A3 | L12-B2 | L12-B2 |
| 101207 | A3 | L12-B3 | L12-B3 |
| 101208 | A3 | L12-B4 | L12-B4 |
| 101209 | A3 | L12-B5 | L12-B5 |
| 101210 | A3 | L12-B6 | L12-B6 |
| 101211 | A3 | L12-B7 | L12-B7 |
| 101212 | A3 | L12-B8 | L12-B8 |
| 101213 | A3 | L12-B9 | L12-B9 |
| 101214 | A3 | L12-B10 | L12-B10 |
| 101215 | A3 | L12-B11 | L12-B11 |
| 101216 | A3 | L12-B12 | L12-B12 |
| 101217 | A3 | L12-B13 | L12-B13 |
| 101218 | A3 | L12-B14 | L12-B14 |
| 101219 | A3 | L13-B1 | L13-B1 |
| 101220 | A3 | L13-B2 | L13-B2 |
| 101221 | A3 | L13-B3 | L13-B3 |
| 101222 | A3 | L13-B4 | L13-B4 |
| 101223 | A3 | L13-B5 | L13-B5 |
| 101224 | A3 | L13-B6 | L13-B6 |
| 101225 | A3 | L13-B7 | L13-B7 |
| 101226 | A3 | L13-B8 | L13-B8 |
| 101227 | A3 | L13-B9 | L13-B9 |
| 101228 | A3 | L13-B10 | L13-B10 |
| 101229 | A3 | L13-B11 | L13-B11 |
| 101230 | A3 | L13-B12 | L13-B12 |
| 101231 | A3 | L13-B13 | L13-B13 |
| 101232 | A3 | L13-B14 | L13-B14 |
| 101233 | A3 | L14-B1 | L14-B1 |
| 101234 | A3 | L14-B2 | L14-B2 |
| 101235 | A3 | L14-B3 | L14-B3 |
| 101236 | A3 | L14-B4 | L14-B4 |
| 101237 | A3 | L14-B5 | L14-B5 |
| 101238 | A3 | L14-B6 | L14-B6 |
| 101239 | A3 | L14-B7 | L14-B7 |
| 101240 | A3 | L14-B8 | L14-B8 |
| 101241 | A3 | L14-B9 | L14-B9 |
| 101242 | A3 | L14-B10 | L14-B10 |
| 101243 | A3 | L14-B11 | L14-B11 |
| 101244 | A3 | L14-B12 | L14-B12 |
| 101245 | A3 | L14-B13 | L14-B13 |
| 101246 | A3 | L14-B14 | L14-B14 |
| 101247 | A3 | L15-B1 | L15-B1 |
| 101248 | A3 | L15-B2 | L15-B2 |
| 101249 | A3 | L15-B3 | L15-B3 |
| 101250 | A3 | L15-B4 | L15-B4 |
| 101251 | A3 | L15-B5 | L15-B5 |
| 101252 | A3 | L15-B6 | L15-B6 |
| 101253 | A3 | L15-B7 | L15-B7 |
| 101254 | A3 | L15-B8 | L15-B8 |
| 101255 | A3 | L15-B9 | L15-B9 |
| 101256 | A3 | L15-B10 | L15-B10 |
| 101257 | A3 | L15-B11 | L15-B11 |
| 101258 | A3 | L15-B12 | L15-B12 |
| 101259 | A3 | L15-B13 | L15-B13 |
| 101260 | A3 | L15-B14 | L15-B14 |
| 101261 | A4 | A4 | L1-B1 |
| 101262 | A4 | A4 | L1-B2 |
| 101263 | A4 | A4 | L1-B3 |
| 101264 | A4 | A4 | L1-B4 |
| 101265 | A4 | A4 | L1-B5 |
| 101266 | A4 | A4 | L1-B6 |
| 101267 | A4 | A4 | L1-B7 |
| 101268 | A4 | A4 | L1-B8 |
| 101269 | A4 | A4 | L1-B9 |
| 101270 | A4 | A4 | L1-B10 |
| 101271 | A4 | A4 | L1-B11 |
| 101272 | A4 | A4 | L1-B12 |
| 101273 | A4 | A4 | L1-B13 |
| 101274 | A4 | A4 | L1-B14 |
| 101275 | A4 | A4 | L2-B1 |
| 101276 | A4 | A4 | L2-B2 |
| 101277 | A4 | A4 | L2-B3 |
| 101278 | A4 | A4 | L2-B4 |
| 101279 | A4 | A4 | L2-B5 |
| 101280 | A4 | A4 | L2-B6 |
| 101281 | A4 | A4 | L2-B7 |
| 101282 | A4 | A4 | L2-B8 |
| 101283 | A4 | A4 | L2-B9 |
| 101284 | A4 | A4 | L2-B10 |
| 101285 | A4 | A4 | L2-B11 |
| 101286 | A4 | A4 | L2-B12 |
| 101287 | A4 | A4 | L2-B13 |
| 101288 | A4 | A4 | L2-B14 |
| 101289 | A4 | A4 | L3-B1 |
| 101290 | A4 | A4 | L3-B2 |
| 101291 | A4 | A4 | L3-B3 |
| 101292 | A4 | A4 | L3-B4 |
| 101293 | A4 | A4 | L3-B5 |
| 101294 | A4 | A4 | L3-B6 |
| 101295 | A4 | A4 | L3-B7 |
| 101296 | A4 | A4 | L3-B8 |
| 101297 | A4 | A4 | L3-B9 |
| 101298 | A4 | A4 | L3-B10 |
| 101299 | A4 | A4 | L3-B11 |
| 101300 | A4 | A4 | L3-B12 |
| 101301 | A4 | A4 | L3-B13 |
| 101302 | A4 | A4 | L3-B14 |
| 101303 | A4 | A4 | L4-B1 |
| 101304 | A4 | A4 | L4-B2 |
| 101305 | A4 | A4 | L4-B3 |
| 101306 | A4 | A4 | L4-B4 |
| 101307 | A4 | A4 | L4-B5 |
| 101308 | A4 | A4 | L4-B6 |
| 101309 | A4 | A4 | L4-B7 |
| 101310 | A4 | A4 | L4-B8 |
| 101311 | A4 | A4 | L4-B9 |
| 101312 | A4 | A4 | L4-B10 |
| 101313 | A4 | A4 | L4-B11 |
| 101314 | A4 | A4 | L4-B12 |
| 101315 | A4 | A4 | L4-B13 |
| 101316 | A4 | A4 | L4-B14 |
| 101317 | A4 | A4 | L5-B1 |
| 101318 | A4 | A4 | L5-B2 |
| 101319 | A4 | A4 | L5-B3 |
| 101320 | A4 | A4 | L5-B4 |
| 101321 | A4 | A4 | L5-B5 |
| 101322 | A4 | A4 | L5-B6 |
| 101323 | A4 | A4 | L5-B7 |
| 101324 | A4 | A4 | L5-B8 |
| 101325 | A4 | A4 | L5-B9 |
| 101326 | A4 | A4 | L5-B10 |
| 101327 | A4 | A4 | L5-B11 |
| 101328 | A4 | A4 | L5-B12 |
| 101329 | A4 | A4 | L5-B13 |
| 101330 | A4 | A4 | L5-B14 |
| 101331 | A4 | A4 | L6-B1 |
| 101332 | A4 | A4 | L6-B2 |
| 101333 | A4 | A4 | L6-B3 |
| 101334 | A4 | A4 | L6-B4 |
| 101335 | A4 | A4 | L6-B5 |
| 101336 | A4 | A4 | L6-B6 |
| 101337 | A4 | A4 | L6-B7 |
| 101338 | A4 | A4 | L6-B8 |
| 101339 | A4 | A4 | L6-B9 |
| 101340 | A4 | A4 | L6-B10 |
| 101341 | A4 | A4 | L6-B11 |
| 101342 | A4 | A4 | L6-B12 |
| 101343 | A4 | A4 | L6-B13 |
| 101344 | A4 | A4 | L6-B14 |
| 101345 | A4 | A4 | L7-B1 |
| 101346 | A4 | A4 | L7-B2 |
| 101347 | A4 | A4 | L7-B3 |
| 101348 | A4 | A4 | L7-B4 |
| 101349 | A4 | A4 | L7-B5 |
| 101350 | A4 | A4 | L7-B6 |
| 101351 | A4 | A4 | L7-B7 |
| 101352 | A4 | A4 | L7-B8 |
| 101353 | A4 | A4 | L7-B9 |
| 101354 | A4 | A4 | L7-B10 |
| 101355 | A4 | A4 | L7-B11 |
| 101356 | A4 | A4 | L7-B12 |
| 101357 | A4 | A4 | L7-B13 |
| 101358 | A4 | A4 | L7-B14 |
| 101359 | A4 | A4 | L8-B1 |
| 101360 | A4 | A4 | L8-B2 |
| 101361 | A4 | A4 | L8-B3 |
| 101362 | A4 | A4 | L8-B4 |
| 101363 | A4 | A4 | L8-B5 |
| 101364 | A4 | A4 | L8-B6 |
| 101365 | A4 | A4 | L8-B7 |
| 101366 | A4 | A4 | L8-B8 |
| 101367 | A4 | A4 | L8-B9 |
| 101368 | A4 | A4 | L8-B10 |
| 101369 | A4 | A4 | L8-B11 |
| 101370 | A4 | A4 | L8-B12 |
| 101371 | A4 | A4 | L8-B13 |
| 101372 | A4 | A4 | L8-B14 |
| 101373 | A4 | A4 | L9-B1 |
| 101374 | A4 | A4 | L9-B2 |
| 101375 | A4 | A4 | L9-B3 |
| 101376 | A4 | A4 | L9-B4 |
| 101377 | A4 | A4 | L9-B5 |
| 101378 | A4 | A4 | L9-B6 |
| 101379 | A4 | A4 | L9-B7 |
| 101380 | A4 | A4 | L9-B8 |
| 101381 | A4 | A4 | L9-B9 |
| 101382 | A4 | A4 | L9-B10 |
| 101383 | A4 | A4 | L9-B11 |
| 101384 | A4 | A4 | L9-B12 |
| 101385 | A4 | A4 | L9-B13 |
| 101386 | A4 | A4 | L9-B14 |
| 101387 | A4 | A4 | L10-B1 |
| 101388 | A4 | A4 | L10-B2 |
| 101389 | A4 | A4 | L10-B3 |
| 101390 | A4 | A4 | L10-B4 |
| 101391 | A4 | A4 | L10-B5 |
| 101392 | A4 | A4 | L10-B6 |
| 101393 | A4 | A4 | L10-B7 |
| 101394 | A4 | A4 | L10-B8 |
| 101395 | A4 | A4 | L10-B9 |
| 101396 | A4 | A4 | L10-B10 |
| 101397 | A4 | A4 | L10-B11 |
| 101398 | A4 | A4 | L10-B12 |
| 101399 | A4 | A4 | L10-B13 |
| 101400 | A4 | A4 | L10-B14 |
| 101401 | A4 | A4 | L11-B1 |
| 101402 | A4 | A4 | L11-B2 |
| 101403 | A4 | A4 | L11-B3 |
| 101404 | A4 | A4 | L11-B4 |
| 101405 | A4 | A4 | L11-B5 |
| 101406 | A4 | A4 | L11-B6 |
| 101407 | A4 | A4 | L11-B7 |
| 101408 | A4 | A4 | L11-B8 |
| 101409 | A4 | A4 | L11-B9 |
| 101410 | A4 | A4 | L11-B10 |
| 101411 | A4 | A4 | L11-B11 |
| 101412 | A4 | A4 | L11-B12 |
| 101413 | A4 | A4 | L11-B13 |
| 101414 | A4 | A4 | L11-B14 |
| 101415 | A4 | A4 | L12-B1 |
| 101416 | A4 | A4 | L12-B2 |
| 101417 | A4 | A4 | L12-B3 |
| 101418 | A4 | A4 | L12-B4 |
| 101419 | A4 | A4 | L12-B5 |
| 101420 | A4 | A4 | L12-B6 |
| 101421 | A4 | A4 | L12-B7 |
| 101422 | A4 | A4 | L12-B8 |
| 101423 | A4 | A4 | L12-B9 |
| 101424 | A4 | A4 | L12-B10 |
| 101425 | A4 | A4 | L12-B11 |
| 101426 | A4 | A4 | L12-B12 |
| 101427 | A4 | A4 | L12-B13 |
| 101428 | A4 | A4 | L12-B14 |
| 101429 | A4 | A4 | L13-B1 |
| 101430 | A4 | A4 | L13-B2 |
| 101431 | A4 | A4 | L13-B3 |
| 101432 | A4 | A4 | L13-B4 |
| 101433 | A4 | A4 | L13-B5 |
| 101434 | A4 | A4 | L13-B6 |
| 101435 | A4 | A4 | L13-B7 |
| 101436 | A4 | A4 | L13-B8 |
| 101437 | A4 | A4 | L13-B9 |
| 101438 | A4 | A4 | L13-B10 |
| 101439 | A4 | A4 | L13-B11 |
| 101440 | A4 | A4 | L13-B12 |
| 101441 | A4 | A4 | L13-B13 |
| 101442 | A4 | A4 | L13-B14 |
| 101443 | A4 | A4 | L14-B1 |
| 101444 | A4 | A4 | L14-B2 |
| 101445 | A4 | A4 | L14-B3 |
| 101446 | A4 | A4 | L14-B4 |
| 101447 | A4 | A4 | L14-B5 |
| 101448 | A4 | A4 | L14-B6 |
| 101449 | A4 | A4 | L14-B7 |
| 101450 | A4 | A4 | L14-B8 |
| 101451 | A4 | A4 | L14-B9 |
| 101452 | A4 | A4 | L14-B10 |
| 101453 | A4 | A4 | L14-B11 |
| 101454 | A4 | A4 | L14-B12 |
| 101455 | A4 | A4 | L14-B13 |
| 101456 | A4 | A4 | L14-B14 |
| 101457 | A4 | A4 | L15-B1 |
| 101458 | A4 | A4 | L15-B2 |
| 101459 | A4 | A4 | L15-B3 |
| 101460 | A4 | A4 | L15-B4 |
| 101461 | A4 | A4 | L15-B5 |
| 101462 | A4 | A4 | L15-B6 |
| 101463 | A4 | A4 | L15-B7 |
| 101464 | A4 | A4 | L15-B8 |
| 101465 | A4 | A4 | L15-B9 |
| 101466 | A4 | A4 | L15-B10 |
| 101467 | A4 | A4 | L15-B11 |
| 101468 | A4 | A4 | L15-B12 |
| 101469 | A4 | A4 | L15-B13 |
| 101470 | A4 | A4 | L15-B14 |
| 101471 | A4 | L1-B1 | L1-B1 |
| 101472 | A4 | L1-B2 | L1-B2 |
| 101473 | A4 | L1-B3 | L1-B3 |
| 101474 | A4 | L1-B4 | L1-B4 |
| 101475 | A4 | L1-B5 | L1-B5 |
| 101476 | A4 | L1-B6 | L1-B6 |
| 101477 | A4 | L1-B7 | L1-B7 |
| 101478 | A4 | L1-B8 | L1-B8 |
| 101479 | A4 | L1-B9 | L1-B9 |
| 101480 | A4 | L1-B10 | L1-B10 |
| 101481 | A4 | L1-B11 | L1-B11 |
| 101482 | A4 | L1-B12 | L1-B12 |
| 101483 | A4 | L1-B13 | L1-B13 |
| 101484 | A4 | L1-B14 | L1-B14 |
| 101485 | A4 | L2-B1 | L2-B1 |
| 101486 | A4 | L2-B2 | L2-B2 |
| 101487 | A4 | L2-B3 | L2-B3 |
| 101488 | A4 | L2-B4 | L2-B4 |
| 101489 | A4 | L2-B5 | L2-B5 |
| 101490 | A4 | L2-B6 | L2-B6 |
| 101491 | A4 | L2-B7 | L2-B7 |
| 101492 | A4 | L2-B8 | L2-B8 |
| 101493 | A4 | L2-B9 | L2-B9 |
| 101494 | A4 | L2-B10 | L2-B10 |
| 101495 | A4 | L2-B11 | L2-B11 |
| 101496 | A4 | L2-B12 | L2-B12 |
| 101497 | A4 | L2-B13 | L2-B13 |
| 101498 | A4 | L2-B14 | L2-B14 |
| 101499 | A4 | L3-B1 | L3-B1 |
| 101500 | A4 | L3-B2 | L3-B2 |
| 101501 | A4 | L3-B3 | L3-B3 |
| 101502 | A4 | L3-B4 | L3-B4 |
| 101503 | A4 | L3-B5 | L3-B5 |
| 101504 | A4 | L3-B6 | L3-B6 |
| 101505 | A4 | L3-B7 | L3-B7 |
| 101506 | A4 | L3-B8 | L3-B8 |
| 101507 | A4 | L3-B9 | L3-B9 |
| 101508 | A4 | L3-B10 | L3-B10 |
| 101509 | A4 | L3-B11 | L3-B11 |
| 101510 | A4 | L3-B12 | L3-B12 |
| 101511 | A4 | L3-B13 | L3-B13 |
| 101512 | A4 | L3-B14 | L3-B14 |
| 101513 | A4 | L4-B1 | L4-B1 |
| 101514 | A4 | L4-B2 | L4-B2 |
| 101515 | A4 | L4-B3 | L4-B3 |
| 101516 | A4 | L4-B4 | L4-B4 |
| 101517 | A4 | L4-B5 | L4-B5 |
| 101518 | A4 | L4-B6 | L4-B6 |
| 101519 | A4 | L4-B7 | L4-B7 |
| 101520 | A4 | L4-B8 | L4-B8 |
| 101521 | A4 | L4-B9 | L4-B9 |
| 101522 | A4 | L4-B10 | L4-B10 |
| 101523 | A4 | L4-B11 | L4-B11 |
| 101524 | A4 | L4-B12 | L4-B12 |
| 101525 | A4 | L4-B13 | L4-B13 |
| 101526 | A4 | L4-B14 | L4-B14 |
| 101527 | A4 | L5-B1 | L5-B1 |
| 101528 | A4 | L5-B2 | L5-B2 |
| 101529 | A4 | L5-B3 | L5-B3 |
| 101530 | A4 | L5-B4 | L5-B4 |
| 101531 | A4 | L5-B5 | L5-B5 |
| 101532 | A4 | L5-B6 | L5-B6 |
| 101533 | A4 | L5-B7 | L5-B7 |
| 101534 | A4 | L5-B8 | L5-B8 |
| 101535 | A4 | L5-B9 | L5-B9 |
| 101536 | A4 | L5-B10 | L5-B10 |
| 101537 | A4 | L5-B11 | L5-B11 |
| 101538 | A4 | L5-B12 | L5-B12 |
| 101539 | A4 | L5-B13 | L5-B13 |
| 101540 | A4 | L5-B14 | L5-B14 |
| 101541 | A4 | L6-B1 | L6-B1 |
| 101542 | A4 | L6-B2 | L6-B2 |
| 101543 | A4 | L6-B3 | L6-B3 |
| 101544 | A4 | L6-B4 | L6-B4 |
| 101545 | A4 | L6-B5 | L6-B5 |
| 101546 | A4 | L6-B6 | L6-B6 |
| 101547 | A4 | L6-B7 | L6-B7 |
| 101548 | A4 | L6-B8 | L6-B8 |
| 101549 | A4 | L6-B9 | L6-B9 |
| 101550 | A4 | L6-B10 | L6-B10 |
| 101551 | A4 | L6-B11 | L6-B11 |
| 101552 | A4 | L6-B12 | L6-B12 |
| 101553 | A4 | L6-B13 | L6-B13 |
| 101554 | A4 | L6-B14 | L6-B14 |
| 101555 | A4 | L7-B1 | L7-B1 |
| 101556 | A4 | L7-B2 | L7-B2 |
| 101557 | A4 | L7-B3 | L7-B3 |
| 101558 | A4 | L7-B4 | L7-B4 |
| 101559 | A4 | L7-B5 | L7-B5 |
| 101560 | A4 | L7-B6 | L7-B6 |
| 101561 | A4 | L7-B7 | L7-B7 |
| 101562 | A4 | L7-B8 | L7-B8 |
| 101563 | A4 | L7-B9 | L7-B9 |
| 101564 | A4 | L7-B10 | L7-B10 |
| 101565 | A4 | L7-B11 | L7-B11 |
| 101566 | A4 | L7-B12 | L7-B12 |
| 101567 | A4 | L7-B13 | L7-B13 |
| 101568 | A4 | L7-B14 | L7-B14 |
| 101569 | A4 | L8-B1 | L8-B1 |
| 101570 | A4 | L8-B2 | L8-B2 |
| 101571 | A4 | L8-B3 | L8-B3 |
| 101572 | A4 | L8-B4 | L8-B4 |
| 101573 | A4 | L8-B5 | L8-B5 |
| 101574 | A4 | L8-B6 | L8-B6 |
| 101575 | A4 | L8-B7 | L8-B7 |
| 101576 | A4 | L8-B8 | L8-B8 |
| 101577 | A4 | L8-B9 | L8-B9 |
| 101578 | A4 | L8-B10 | L8-B10 |
| 101579 | A4 | L8-B11 | L8-B11 |
| 101580 | A4 | L8-B12 | L8-B12 |
| 101581 | A4 | L8-B13 | L8-B13 |
| 101582 | A4 | L8-B14 | L8-B14 |
| 101583 | A4 | L9-B1 | L9-B1 |
| 101584 | A4 | L9-B2 | L9-B2 |
| 101585 | A4 | L9-B3 | L9-B3 |
| 101586 | A4 | L9-B4 | L9-B4 |
| 101587 | A4 | L9-B5 | L9-B5 |
| 101588 | A4 | L9-B6 | L9-B6 |
| 101589 | A4 | L9-B7 | L9-B7 |
| 101590 | A4 | L9-B8 | L9-B8 |
| 101591 | A4 | L9-B9 | L9-B9 |
| 101592 | A4 | L9-B10 | L9-B10 |
| 101593 | A4 | L9-B11 | L9-B11 |
| 101594 | A4 | L9-B12 | L9-B12 |
| 101595 | A4 | L9-B13 | L9-B13 |
| 101596 | A4 | L9-B14 | L9-B14 |
| 101597 | A4 | L10-B1 | L10-B1 |
| 101598 | A4 | L10-B2 | L10-B2 |
| 101599 | A4 | L10-B3 | L10-B3 |
| 101600 | A4 | L10-B4 | L10-B4 |
| 101601 | A4 | L10-B5 | L10-B5 |
| 101602 | A4 | L10-B6 | L10-B6 |
| 101603 | A4 | L10-B7 | L10-B7 |
| 101604 | A4 | L10-B8 | L10-B8 |
| 101605 | A4 | L10-B9 | L10-B9 |
| 101606 | A4 | L10-B10 | L10-B10 |
| 101607 | A4 | L10-B11 | L10-B11 |
| 101608 | A4 | L10-B12 | L10-B12 |
| 101609 | A4 | L10-B13 | L10-B13 |
| 101610 | A4 | L10-B14 | L10-B14 |
| 101611 | A4 | L11-B1 | L11-B1 |
| 101612 | A4 | L11-B2 | L11-B2 |
| 101613 | A4 | L11-B3 | L11-B3 |
| 101614 | A4 | L11-B4 | L11-B4 |
| 101615 | A4 | L11-B5 | L11-B5 |
| 101616 | A4 | L11-B6 | L11-B6 |
| 101617 | A4 | L11-B7 | L11-B7 |
| 101618 | A4 | L11-B8 | L11-B8 |
| 101619 | A4 | L11-B9 | L11-B9 |
| 101620 | A4 | L11-B10 | L11-B10 |
| 101621 | A4 | L11-B11 | L11-B11 |
| 101622 | A4 | L11-B12 | L11-B12 |
| 101623 | A4 | L11-B13 | L11-B13 |
| 101624 | A4 | L11-B14 | L11-B14 |
| 101625 | A4 | L12-B1 | L12-B1 |
| 101626 | A4 | L12-B2 | L12-B2 |
| 101627 | A4 | L12-B3 | L12-B3 |
| 101628 | A4 | L12-B4 | L12-B4 |
| 101629 | A4 | L12-B5 | L12-B5 |
| 101630 | A4 | L12-B6 | L12-B6 |
| 101631 | A4 | L12-B7 | L12-B7 |
| 101632 | A4 | L12-B8 | L12-B8 |
| 101633 | A4 | L12-B9 | L12-B9 |
| 101634 | A4 | L12-B10 | L12-B10 |
| 101635 | A4 | L12-B11 | L12-B11 |
| 101636 | A4 | L12-B12 | L12-B12 |
| 101637 | A4 | L12-B13 | L12-B13 |
| 101638 | A4 | L12-B14 | L12-B14 |
| 101639 | A4 | L13-B1 | L13-B1 |
| 101640 | A4 | L13-B2 | L13-B2 |
| 101641 | A4 | L13-B3 | L13-B3 |
| 101642 | A4 | L13-B4 | L13-B4 |
| 101643 | A4 | L13-B5 | L13-B5 |
| 101644 | A4 | L13-B6 | L13-B6 |
| 101645 | A4 | L13-B7 | L13-B7 |
| 101646 | A4 | L13-B8 | L13-B8 |
| 101647 | A4 | L13-B9 | L13-B9 |
| 101648 | A4 | L13-B10 | L13-B10 |
| 101649 | A4 | L13-B11 | L13-B11 |
| 101650 | A4 | L13-B12 | L13-B12 |
| 101651 | A4 | L13-B13 | L13-B13 |
| 101652 | A4 | L13-B14 | L13-B14 |
| 101653 | A4 | L14-B1 | L14-B1 |
| 101654 | A4 | L14-B2 | L14-B2 |
| 101655 | A4 | L14-B3 | L14-B3 |
| 101656 | A4 | L14-B4 | L14-B4 |
| 101657 | A4 | L14-B5 | L14-B5 |
| 101658 | A4 | L14-B6 | L14-B6 |
| 101659 | A4 | L14-B7 | L14-B7 |
| 101660 | A4 | L14-B8 | L14-B8 |
| 101661 | A4 | L14-B9 | L14-B9 |
| 101662 | A4 | L14-B10 | L14-B10 |
| 101663 | A4 | L14-B11 | L14-B11 |
| 101664 | A4 | L14-B12 | L14-B12 |
| 101665 | A4 | L14-B13 | L14-B13 |
| 101666 | A4 | L14-B14 | L14-B14 |
| 101667 | A4 | L15-B1 | L15-B1 |
| 101668 | A4 | L15-B2 | L15-B2 |
| 101669 | A4 | L15-B3 | L15-B3 |
| 101670 | A4 | L15-B4 | L15-B4 |
| 101671 | A4 | L15-B5 | L15-B5 |
| 101672 | A4 | L15-B6 | L15-B6 |
| 101673 | A4 | L15-B7 | L15-B7 |
| 101674 | A4 | L15-B8 | L15-B8 |
| 101675 | A4 | L15-B9 | L15-B9 |
| 101676 | A4 | L15-B10 | L15-B10 |
| 101677 | A4 | L15-B11 | L15-B11 |
| 101678 | A4 | L15-B12 | L15-B12 |
| 101679 | A4 | L15-B13 | L15-B13 |
| 101680 | A4 | L15-B14 | L15-B14 |
| 101681 | A5 | A5 | L1-B1 |
| 101682 | A5 | A5 | L1-B2 |
| 101683 | A5 | A5 | L1-B3 |
| 101684 | A5 | A5 | L1-B4 |
| 101685 | A5 | A5 | L1-B5 |
| 101686 | A5 | A5 | L1-B6 |
| 101687 | A5 | A5 | L1-B7 |
| 101688 | A5 | A5 | L1-B8 |
| 101689 | A5 | A5 | L1-B9 |
| 101690 | A5 | A5 | L1-B10 |
| 101691 | A5 | A5 | L1-B11 |
| 101692 | A5 | A5 | L1-B12 |
| 101693 | A5 | A5 | L1-B13 |
| 101694 | A5 | A5 | L1-B14 |
| 101695 | A5 | A5 | L2-B1 |
| 101696 | A5 | A5 | L2-B2 |
| 101697 | A5 | A5 | L2-B3 |
| 101698 | A5 | A5 | L2-B4 |
| 101699 | A5 | A5 | L2-B5 |
| 101700 | A5 | A5 | L2-B6 |
| 101701 | A5 | A5 | L2-B7 |
| 101702 | A5 | A5 | L2-B8 |
| 101703 | A5 | A5 | L2-B9 |
| 101704 | A5 | A5 | L2-B10 |
| 101705 | A5 | A5 | L2-B11 |
| 101706 | A5 | A5 | L2-B12 |
| 101707 | A5 | A5 | L2-B13 |
| 101708 | A5 | A5 | L2-B14 |
| 101709 | A5 | A5 | L3-B1 |
| 101710 | A5 | A5 | L3-B2 |
| 101711 | A5 | A5 | L3-B3 |
| 101712 | A5 | A5 | L3-B4 |
| 101713 | A5 | A5 | L3-B5 |
| 101714 | A5 | A5 | L3-B6 |
| 101715 | A5 | A5 | L3-B7 |
| 101716 | A5 | A5 | L3-B8 |
| 101717 | A5 | A5 | L3-B9 |
| 101718 | A5 | A5 | L3-B10 |
| 101719 | A5 | A5 | L3-B11 |
| 101720 | A5 | A5 | L3-B12 |
| 101721 | A5 | A5 | L3-B13 |
| 101722 | A5 | A5 | L3-B14 |
| 101723 | A5 | A5 | L4-B1 |
| 101724 | A5 | A5 | L4-B2 |
| 101725 | A5 | A5 | L4-B3 |
| 101726 | A5 | A5 | L4-B4 |
| 101727 | A5 | A5 | L4-B5 |
| 101728 | A5 | A5 | L4-B6 |
| 101729 | A5 | A5 | L4-B7 |
| 101730 | A5 | A5 | L4-B8 |
| 101731 | A5 | A5 | L4-B9 |
| 101732 | A5 | A5 | L4-B10 |
| 101733 | A5 | A5 | L4-B11 |
| 101734 | A5 | A5 | L4-B12 |
| 101735 | A5 | A5 | L4-B13 |
| 101736 | A5 | A5 | L4-B14 |
| 101737 | A5 | A5 | L5-B1 |
| 101738 | A5 | A5 | L5-B2 |
| 101739 | A5 | A5 | L5-B3 |
| 101740 | A5 | A5 | L5-B4 |
| 101741 | A5 | A5 | L5-B5 |
| 101742 | A5 | A5 | L5-B6 |
| 101743 | A5 | A5 | L5-B7 |
| 101744 | A5 | A5 | L5-B8 |
| 101745 | A5 | A5 | L5-B9 |
| 101746 | A5 | A5 | L5-B10 |
| 101747 | A5 | A5 | L5-B11 |
| 101748 | A5 | A5 | L5-B12 |
| 101749 | A5 | A5 | L5-B13 |
| 101750 | A5 | A5 | L5-B14 |
| 101751 | A5 | A5 | L6-B1 |
| 101752 | A5 | A5 | L6-B2 |
| 101753 | A5 | A5 | L6-B3 |
| 101754 | A5 | A5 | L6-B4 |
| 101755 | A5 | A5 | L6-B5 |
| 101756 | A5 | A5 | L6-B6 |
| 101757 | A5 | A5 | L6-B7 |
| 101758 | A5 | A5 | L6-B8 |
| 101759 | A5 | A5 | L6-B9 |
| 101760 | A5 | A5 | L6-B10 |
| 101761 | A5 | A5 | L6-B11 |
| 101762 | A5 | A5 | L6-B12 |
| 101763 | A5 | A5 | L6-B13 |
| 101764 | A5 | A5 | L6-B14 |
| 101765 | A5 | A5 | L7-B1 |
| 101766 | A5 | A5 | L7-B2 |
| 101767 | A5 | A5 | L7-B3 |
| 101768 | A5 | A5 | L7-B4 |
| 101769 | A5 | A5 | L7-B5 |
| 101770 | A5 | A5 | L7-B6 |
| 101771 | A5 | A5 | L7-B7 |
| 101772 | A5 | A5 | L7-B8 |
| 101773 | A5 | A5 | L7-B9 |
| 101774 | A5 | A5 | L7-B10 |
| 101775 | A5 | A5 | L7-B11 |
| 101776 | A5 | A5 | L7-B12 |
| 101777 | A5 | A5 | L7-B13 |
| 101778 | A5 | A5 | L7-B14 |
| 101779 | A5 | A5 | L8-B1 |
| 101780 | A5 | A5 | L8-B2 |
| 101781 | A5 | A5 | L8-B3 |
| 101782 | A5 | A5 | L8-B4 |
| 101783 | A5 | A5 | L8-B5 |
| 101784 | A5 | A5 | L8-B6 |
| 101785 | A5 | A5 | L8-B7 |
| 101786 | A5 | A5 | L8-B8 |
| 101787 | A5 | A5 | L8-B9 |
| 101788 | A5 | A5 | L8-B10 |
| 101789 | A5 | A5 | L8-B11 |
| 101790 | A5 | A5 | L8-B12 |
| 101791 | A5 | A5 | L8-B13 |
| 101792 | A5 | A5 | L8-B14 |
| 101793 | A5 | A5 | L9-B1 |
| 101794 | A5 | A5 | L9-B2 |
| 101795 | A5 | A5 | L9-B3 |
| 101796 | A5 | A5 | L9-B4 |
| 101797 | A5 | A5 | L9-B5 |
| 101798 | A5 | A5 | L9-B6 |
| 101799 | A5 | A5 | L9-B7 |
| 101800 | A5 | A5 | L9-B8 |
| 101801 | A5 | A5 | L9-B9 |
| 101802 | A5 | A5 | L9-B10 |
| 101803 | A5 | A5 | L9-B11 |
| 101804 | A5 | A5 | L9-B12 |
| 101805 | A5 | A5 | L9-B13 |
| 101806 | A5 | A5 | L9-B14 |
| 101807 | A5 | A5 | L10-B1 |
| 101808 | A5 | A5 | L10-B2 |
| 101809 | A5 | A5 | L10-B3 |
| 101810 | A5 | A5 | L10-B4 |
| 101811 | A5 | A5 | L10-B5 |
| 101812 | A5 | A5 | L10-B6 |
| 101813 | A5 | A5 | L10-B7 |
| 101814 | A5 | A5 | L10-B8 |
| 101815 | A5 | A5 | L10-B9 |
| 101816 | A5 | A5 | L10-B10 |
| 101817 | A5 | A5 | L10-B11 |
| 101818 | A5 | A5 | L10-B12 |
| 101819 | A5 | A5 | L10-B13 |
| 101820 | A5 | A5 | L10-B14 |
| 101821 | A5 | A5 | L11-B1 |
| 101822 | A5 | A5 | L11-B2 |
| 101823 | A5 | A5 | L11-B3 |
| 101824 | A5 | A5 | L11-B4 |
| 101825 | A5 | A5 | L11-B5 |
| 101826 | A5 | A5 | L11-B6 |
| 101827 | A5 | A5 | L11-B7 |
| 101828 | A5 | A5 | L11-B8 |
| 101829 | A5 | A5 | L11-B9 |
| 101830 | A5 | A5 | L11-B10 |
| 101831 | A5 | A5 | L11-B11 |
| 101832 | A5 | A5 | L11-B12 |
| 101833 | A5 | A5 | L11-B13 |
| 101834 | A5 | A5 | L11-B14 |
| 101835 | A5 | A5 | L12-B1 |
| 101836 | A5 | A5 | L12-B2 |
| 101837 | A5 | A5 | L12-B3 |
| 101838 | A5 | A5 | L12-B4 |
| 101839 | A5 | A5 | L12-B5 |
| 101840 | A5 | A5 | L12-B6 |
| 101841 | A5 | A5 | L12-B7 |
| 101842 | A5 | A5 | L12-B8 |
| 101843 | A5 | A5 | L12-B9 |
| 101844 | A5 | A5 | L12-B10 |
| 101845 | A5 | A5 | L12-B11 |
| 101846 | A5 | A5 | L12-B12 |
| 101847 | A5 | A5 | L12-B13 |
| 101848 | A5 | A5 | L12-B14 |
| 101849 | A5 | A5 | L13-B1 |
| 101850 | A5 | A5 | L13-B2 |
| 101851 | A5 | A5 | L13-B3 |
| 101852 | A5 | A5 | L13-B4 |
| 101853 | A5 | A5 | L13-B5 |
| 101854 | A5 | A5 | L13-B6 |
| 101855 | A5 | A5 | L13-B7 |
| 101856 | A5 | A5 | L13-B8 |
| 101857 | A5 | A5 | L13-B9 |
| 101858 | A5 | A5 | L13-B10 |
| 101859 | A5 | A5 | L13-B11 |
| 101860 | A5 | A5 | L13-B12 |
| 101861 | A5 | A5 | L13-B13 |
| 101862 | A5 | A5 | L13-B14 |
| 101863 | A5 | A5 | L14-B1 |
| 101864 | A5 | A5 | L14-B2 |
| 101865 | A5 | A5 | L14-B3 |
| 101866 | A5 | A5 | L14-B4 |
| 101867 | A5 | A5 | L14-B5 |
| 101868 | A5 | A5 | L14-B6 |
| 101869 | A5 | A5 | L14-B7 |
| 101870 | A5 | A5 | L14-B8 |
| 101871 | A5 | A5 | L14-B9 |
| 101872 | A5 | A5 | L14-B10 |
| 101873 | A5 | A5 | L14-B11 |
| 101874 | A5 | A5 | L14-B12 |
| 101875 | A5 | A5 | L14-B13 |
| 101876 | A5 | A5 | L14-B14 |
| 101877 | A5 | A5 | L15-B1 |
| 101878 | A5 | A5 | L15-B2 |
| 101879 | A5 | A5 | L15-B3 |
| 101880 | A5 | A5 | L15-B4 |
| 101881 | A5 | A5 | L15-B5 |
| 101882 | A5 | A5 | L15-B6 |
| 101883 | A5 | A5 | L15-B7 |
| 101884 | A5 | A5 | L15-B8 |
| 101885 | A5 | A5 | L15-B9 |
| 101886 | A5 | A5 | L15-B10 |
| 101887 | A5 | A5 | L15-B11 |
| 101888 | A5 | A5 | L15-B12 |
| 101889 | A5 | A5 | L15-B13 |
| 101890 | A5 | A5 | L15-B14 |
| 101891 | A5 | L1-B1 | L1-B1 |
| 101892 | A5 | L1-B2 | L1-B2 |
| 101893 | A5 | L1-B3 | L1-B3 |
| 101894 | A5 | L1-B4 | L1-B4 |
| 101895 | A5 | L1-B5 | L1-B5 |
| 101896 | A5 | L1-B6 | L1-B6 |
| 101897 | A5 | L1-B7 | L1-B7 |
| 101898 | A5 | L1-B8 | L1-B8 |
| 101899 | A5 | L1-B9 | L1-B9 |
| 101900 | A5 | L1-B10 | L1-B10 |
| 101901 | A5 | L1-B11 | L1-B11 |
| 101902 | A5 | L1-B12 | L1-B12 |
| 101903 | A5 | L1-B13 | L1-B13 |
| 101904 | A5 | L1-B14 | L1-B14 |
| 101905 | A5 | L2-B1 | L2-B1 |
| 101906 | A5 | L2-B2 | L2-B2 |
| 101907 | A5 | L2-B3 | L2-B3 |
| 101908 | A5 | L2-B4 | L2-B4 |
| 101909 | A5 | L2-B5 | L2-B5 |
| 101910 | A5 | L2-B6 | L2-B6 |
| 101911 | A5 | L2-B7 | L2-B7 |
| 101912 | A5 | L2-B8 | L2-B8 |
| 101913 | A5 | L2-B9 | L2-B9 |
| 101914 | A5 | L2-B10 | L2-B10 |
| 101915 | A5 | L2-B11 | L2-B11 |
| 101916 | A5 | L2-B12 | L2-B12 |
| 101917 | A5 | L2-B13 | L2-B13 |
| 101918 | A5 | L2-B14 | L2-B14 |
| 101919 | A5 | L3-B1 | L3-B1 |
| 101920 | A5 | L3-B2 | L3-B2 |
| 101921 | A5 | L3-B3 | L3-B3 |
| 101922 | A5 | L3-B4 | L3-B4 |
| 101923 | A5 | L3-B5 | L3-B5 |
| 101924 | A5 | L3-B6 | L3-B6 |
| 101925 | A5 | L3-B7 | L3-B7 |
| 101926 | A5 | L3-B8 | L3-B8 |
| 101927 | A5 | L3-B9 | L3-B9 |
| 101928 | A5 | L3-B10 | L3-B10 |
| 101929 | A5 | L3-B11 | L3-B11 |
| 101930 | A5 | L3-B12 | L3-B12 |
| 101931 | A5 | L3-B13 | L3-B13 |
| 101932 | A5 | L3-B14 | L3-B14 |
| 101933 | A5 | L4-B1 | L4-B1 |
| 101934 | A5 | L4-B2 | L4-B2 |
| 101935 | A5 | L4-B3 | L4-B3 |
| 101936 | A5 | L4-B4 | L4-B4 |
| 101937 | A5 | L4-B5 | L4-B5 |
| 101938 | A5 | L4-B6 | L4-B6 |
| 101939 | A5 | L4-B7 | L4-B7 |
| 101940 | A5 | L4-B8 | L4-B8 |
| 101941 | A5 | L4-B9 | L4-B9 |
| 101942 | A5 | L4-B10 | L4-B10 |
| 101943 | A5 | L4-B11 | L4-B11 |
| 101944 | A5 | L4-B12 | L4-B12 |
| 101945 | A5 | L4-B13 | L4-B13 |
| 101946 | A5 | L4-B14 | L4-B14 |
| 101947 | A5 | L5-B1 | L5-B1 |
| 101948 | A5 | L5-B2 | L5-B2 |
| 101949 | A5 | L5-B3 | L5-B3 |
| 101950 | A5 | L5-B4 | L5-B4 |
| 101951 | A5 | L5-B5 | L5-B5 |
| 101952 | A5 | L5-B6 | L5-B6 |
| 101953 | A5 | L5-B7 | L5-B7 |
| 101954 | A5 | L5-B8 | L5-B8 |
| 101955 | A5 | L5-B9 | L5-B9 |
| 101956 | A5 | L5-B10 | L5-B10 |
| 101957 | A5 | L5-B11 | L5-B11 |
| 101958 | A5 | L5-B12 | L5-B12 |
| 101959 | A5 | L5-B13 | L5-B13 |
| 101960 | A5 | L5-B14 | L5-B14 |
| 101961 | A5 | L6-B1 | L6-B1 |
| 101962 | A5 | L6-B2 | L6-B2 |
| 101963 | A5 | L6-B3 | L6-B3 |
| 101964 | A5 | L6-B4 | L6-B4 |
| 101965 | A5 | L6-B5 | L6-B5 |
| 101966 | A5 | L6-B6 | L6-B6 |
| 101967 | A5 | L6-B7 | L6-B7 |
| 101968 | A5 | L6-B8 | L6-B8 |
| 101969 | A5 | L6-B9 | L6-B9 |
| 101970 | A5 | L6-B10 | L6-B10 |
| 101971 | A5 | L6-B11 | L6-B11 |
| 101972 | A5 | L6-B12 | L6-B12 |
| 101973 | A5 | L6-B13 | L6-B13 |
| 101974 | A5 | L6-B14 | L6-B14 |
| 101975 | A5 | L7-B1 | L7-B1 |
| 101976 | A5 | L7-B2 | L7-B2 |
| 101977 | A5 | L7-B3 | L7-B3 |
| 101978 | A5 | L7-B4 | L7-B4 |
| 101979 | A5 | L7-B5 | L7-B5 |
| 101980 | A5 | L7-B6 | L7-B6 |
| 101981 | A5 | L7-B7 | L7-B7 |
| 101982 | A5 | L7-B8 | L7-B8 |
| 101983 | A5 | L7-B9 | L7-B9 |
| 101984 | A5 | L7-B10 | L7-B10 |
| 101985 | A5 | L7-B11 | L7-B11 |
| 101986 | A5 | L7-B12 | L7-B12 |
| 101987 | A5 | L7-B13 | L7-B13 |
| 101988 | A5 | L7-B14 | L7-B14 |
| 101989 | A5 | L8-B1 | L8-B1 |
| 101990 | A5 | L8-B2 | L8-B2 |
| 101991 | A5 | L8-B3 | L8-B3 |
| 101992 | A5 | L8-B4 | L8-B4 |
| 101993 | A5 | L8-B5 | L8-B5 |
| 101994 | A5 | L8-B6 | L8-B6 |
| 101995 | A5 | L8-B7 | L8-B7 |
| 101996 | A5 | L8-B8 | L8-B8 |
| 101997 | A5 | L8-B9 | L8-B9 |
| 101998 | A5 | L8-B10 | L8-B10 |
| 101999 | A5 | L8-B11 | L8-B11 |
| 102000 | A5 | L8-B12 | L8-B12 |
| 102001 | A5 | L8-B13 | L8-B13 |
| 102002 | A5 | L8-B14 | L8-B14 |
| 102003 | A5 | L9-B1 | L9-B1 |
| 102004 | A5 | L9-B2 | L9-B2 |
| 102005 | A5 | L9-B3 | L9-B3 |
| 102006 | A5 | L9-B4 | L9-B4 |
| 102007 | A5 | L9-B5 | L9-B5 |
| 102008 | A5 | L9-B6 | L9-B6 |
| 102009 | A5 | L9-B7 | L9-B7 |
| 102010 | A5 | L9-B8 | L9-B8 |
| 102011 | A5 | L9-B9 | L9-B9 |
| 102012 | A5 | L9-B10 | L9-B10 |
| 102013 | A5 | L9-B11 | L9-B11 |
| 102014 | A5 | L9-B12 | L9-B12 |
| 102015 | A5 | L9-B13 | L9-B13 |
| 102016 | A5 | L9-B14 | L9-B14 |
| 102017 | A5 | L10-B1 | L10-B1 |
| 102018 | A5 | L10-B2 | L10-B2 |
| 102019 | A5 | L10-B3 | L10-B3 |
| 102020 | A5 | L10-B4 | L10-B4 |
| 102021 | A5 | L10-B5 | L10-B5 |
| 102022 | A5 | L10-B6 | L10-B6 |
| 102023 | A5 | L10-B7 | L10-B7 |
| 102024 | A5 | L10-B8 | L10-B8 |
| 102025 | A5 | L10-B9 | L10-B9 |
| 102026 | A5 | L10-B10 | L10-B10 |
| 102027 | A5 | L10-B11 | L10-B11 |
| 102028 | A5 | L10-B12 | L10-B12 |
| 102029 | A5 | L10-B13 | L10-B13 |
| 102030 | A5 | L10-B14 | L10-B14 |
| 102031 | A5 | L11-B1 | L11-B1 |
| 102032 | A5 | L11-B2 | L11-B2 |
| 102033 | A5 | L11-B3 | L11-B3 |
| 102034 | A5 | L11-B4 | L11-B4 |
| 102035 | A5 | L11-B5 | L11-B5 |
| 102036 | A5 | L11-B6 | L11-B6 |
| 102037 | A5 | L11-B7 | L11-B7 |
| 102038 | A5 | L11-B8 | L11-B8 |
| 102039 | A5 | L11-B9 | L11-B9 |
| 102040 | A5 | L11-B10 | L11-B10 |
| 102041 | A5 | L11-B11 | LII-B11 |
| 102042 | A5 | L11-B12 | L11-B12 |
| 102043 | A5 | L11-B13 | L11-B13 |
| 102044 | A5 | L11-B14 | L11-B14 |
| 102045 | A5 | L12-B1 | L12-B1 |
| 102046 | A5 | L12-B2 | L12-B2 |
| 102047 | A5 | L12-B3 | L12-B3 |
| 102048 | A5 | L12-B4 | L12-B4 |
| 102049 | A5 | L12-B5 | L12-B5 |
| 102050 | A5 | L12-B6 | L12-B6 |
| 102051 | A5 | L12-B7 | L12-B7 |
| 102052 | A5 | L12-B8 | L12-B8 |
| 102053 | A5 | L12-B9 | L12-B9 |
| 102054 | A5 | L12-B10 | L12-B10 |
| 102055 | A5 | L12-B11 | L12-B11 |
| 102056 | A5 | L12-B12 | L12-B12 |
| 102057 | A5 | L12-B13 | L12-B13 |
| 102058 | A5 | L12-B14 | L12-B14 |
| 102059 | A5 | L13-B1 | L13-B1 |
| 102060 | A5 | L13-B2 | L13-B2 |
| 102061 | A5 | L13-B3 | L13-B3 |
| 102062 | A5 | L13-B4 | L13-B4 |
| 102063 | A5 | L13-B5 | L13-B5 |
| 102064 | A5 | L13-B6 | L13-B6 |
| 102065 | A5 | L13-B7 | L13-B7 |
| 102066 | A5 | L13-B8 | L13-B8 |
| 102067 | A5 | L13-B9 | L13-B9 |
| 102068 | A5 | L13-B10 | L13-B10 |
| 102069 | A5 | L13-B11 | L13-B11 |
| 102070 | A5 | L13-B12 | L13-B12 |
| 102071 | A5 | L13-B13 | L13-B13 |
| 102072 | A5 | L13-B14 | L13-B14 |
| 102073 | A5 | L14-B1 | L14-B1 |
| 102074 | A5 | L14-B2 | L14-B2 |
| 102075 | A5 | L14-B3 | L14-B3 |
| 102076 | A5 | L14-B4 | L14-B4 |
| 102077 | A5 | L14-B5 | L14-B5 |
| 102078 | A5 | L14-B6 | L14-B6 |
| 102079 | A5 | L14-B7 | L14-B7 |
| 102080 | A5 | L14-B8 | L14-B8 |
| 102081 | A5 | L14-B9 | L14-B9 |
| 102082 | A5 | L14-B10 | L14-B10 |
| 102083 | A5 | L14-B11 | L14-B11 |
| 102084 | A5 | L14-B12 | L14-B12 |
| 102085 | A5 | L14-B13 | L14-B13 |
| 102086 | A5 | L14-B14 | L14-B14 |
| 102087 | A5 | L15-B1 | L15-B1 |
| 102088 | A5 | L15-B2 | L15-B2 |
| 102089 | A5 | L15-B3 | L15-B3 |
| 102090 | A5 | L15-B4 | L15-B4 |
| 102091 | A5 | L15-B5 | L15-B5 |
| 102092 | A5 | L15-B6 | L15-B6 |
| 102093 | A5 | L15-B7 | L15-B7 |
| 102094 | A5 | L15-B8 | L15-B8 |
| 102095 | A5 | L15-B9 | L15-B9 |
| 102096 | A5 | L15-B10 | L15-B10 |
| 102097 | A5 | L15-B11 | L15-B11 |
| 102098 | A5 | L15-B12 | L15-B12 |
| 102099 | A5 | L15-B13 | L15-B13 |
| 102100 | A5 | L15-B14 | L15-B14 |
| 102101 | A6 | A6 | L1-B1 |
| 102102 | A6 | A6 | L1-B2 |
| 102103 | A6 | A6 | L1-B3 |
| 102104 | A6 | A6 | L1-B4 |
| 102105 | A6 | A6 | L1-B5 |
| 102106 | A6 | A6 | L1-B6 |
| 102107 | A6 | A6 | L1-B7 |
| 102108 | A6 | A6 | L1-B8 |
| 102109 | A6 | A6 | L1-B9 |
| 102110 | A6 | A6 | L1-B10 |
| 102111 | A6 | A6 | L1-B11 |
| 102112 | A6 | A6 | L1-B12 |
| 102113 | A6 | A6 | L1-B13 |
| 102114 | A6 | A6 | L1-B14 |
| 102115 | A6 | A6 | L2-B1 |
| 102116 | A6 | A6 | L2-B2 |
| 102117 | A6 | A6 | L2-B3 |
| 102118 | A6 | A6 | L2-B4 |
| 102119 | A6 | A6 | L2-B5 |
| 102120 | A6 | A6 | L2-B6 |
| 102121 | A6 | A6 | L2-B7 |
| 102122 | A6 | A6 | L2-B8 |
| 102123 | A6 | A6 | L2-B9 |
| 102124 | A6 | A6 | L2-B10 |
| 102125 | A6 | A6 | L2-B11 |
| 102126 | A6 | A6 | L2-B12 |
| 102127 | A6 | A6 | L2-B13 |
| 102128 | A6 | A6 | L2-B14 |
| 102129 | A6 | A6 | L3-B1 |
| 102130 | A6 | A6 | L3-B2 |
| 102131 | A6 | A6 | L3-B3 |
| 102132 | A6 | A6 | L3-B4 |
| 102133 | A6 | A6 | L3-B5 |
| 102134 | A6 | A6 | L3-B6 |
| 102135 | A6 | A6 | L3-B7 |
| 102136 | A6 | A6 | L3-B8 |
| 102137 | A6 | A6 | L3-B9 |
| 102138 | A6 | A6 | L3-B10 |
| 102139 | A6 | A6 | L3-B11 |
| 102140 | A6 | A6 | L3-B12 |
| 102141 | A6 | A6 | L3-B13 |
| 102142 | A6 | A6 | L3-B14 |
| 102143 | A6 | A6 | L4-B1 |
| 102144 | A6 | A6 | L4-B2 |
| 102145 | A6 | A6 | L4-B3 |
| 102146 | A6 | A6 | L4-B4 |
| 102147 | A6 | A6 | L4-B5 |
| 102148 | A6 | A6 | L4-B6 |
| 102149 | A6 | A6 | L4-B7 |
| 102150 | A6 | A6 | L4-B8 |
| 102151 | A6 | A6 | L4-B9 |
| 102152 | A6 | A6 | L4-B10 |
| 102153 | A6 | A6 | L4-B11 |
| 102154 | A6 | A6 | L4-B12 |
| 102155 | A6 | A6 | L4-B13 |
| 102156 | A6 | A6 | L4-B14 |
| 102157 | A6 | A6 | L5-B1 |
| 102158 | A6 | A6 | L5-B2 |
| 102159 | A6 | A6 | L5-B3 |
| 102160 | A6 | A6 | L5-B4 |
| 102161 | A6 | A6 | L5-B5 |
| 102162 | A6 | A6 | L5-B6 |
| 102163 | A6 | A6 | L5-B7 |
| 102164 | A6 | A6 | L5-B8 |
| 102165 | A6 | A6 | L5-B9 |
| 102166 | A6 | A6 | L5-B10 |
| 102167 | A6 | A6 | L5-B11 |
| 102168 | A6 | A6 | L5-B12 |
| 102169 | A6 | A6 | L5-B13 |
| 102170 | A6 | A6 | L5-B14 |
| 102171 | A6 | A6 | L6-B1 |
| 102172 | A6 | A6 | L6-B2 |
| 102173 | A6 | A6 | L6-B3 |
| 102174 | A6 | A6 | L6-B4 |
| 102175 | A6 | A6 | L6-B5 |
| 102176 | A6 | A6 | L6-B6 |
| 102177 | A6 | A6 | L6-B7 |
| 102178 | A6 | A6 | L6-B8 |
| 102179 | A6 | A6 | L6-B9 |
| 102180 | A6 | A6 | L6-B10 |
| 102181 | A6 | A6 | L6-B11 |
| 102182 | A6 | A6 | L6-B12 |
| 102183 | A6 | A6 | L6-B13 |
| 102184 | A6 | A6 | L6-B14 |
| 102185 | A6 | A6 | L7-B1 |
| 102186 | A6 | A6 | L7-B2 |
| 102187 | A6 | A6 | L7-B3 |
| 102188 | A6 | A6 | L7-B4 |
| 102189 | A6 | A6 | L7-B5 |
| 102190 | A6 | A6 | L7-B6 |
| 102191 | A6 | A6 | L7-B7 |
| 102192 | A6 | A6 | L7-B8 |
| 102193 | A6 | A6 | L7-B9 |
| 102194 | A6 | A6 | L7-B10 |
| 102195 | A6 | A6 | L7-B11 |
| 102196 | A6 | A6 | L7-B12 |
| 102197 | A6 | A6 | L7-B13 |
| 102198 | A6 | A6 | L7-B14 |
| 102199 | A6 | A6 | L8-B1 |
| 102200 | A6 | A6 | L8-B2 |
| 102201 | A6 | A6 | L8-B3 |
| 102202 | A6 | A6 | L8-B4 |
| 102203 | A6 | A6 | L8-B5 |
| 102204 | A6 | A6 | L8-B6 |
| 102205 | A6 | A6 | L8-B7 |
| 102206 | A6 | A6 | L8-B8 |
| 102207 | A6 | A6 | L8-B9 |
| 102208 | A6 | A6 | L8-B10 |
| 102209 | A6 | A6 | L8-B11 |
| 102210 | A6 | A6 | L8-B12 |
| 102211 | A6 | A6 | L8-B13 |
| 102212 | A6 | A6 | L8-B14 |
| 102213 | A6 | A6 | L9-B1 |
| 102214 | A6 | A6 | L9-B2 |
| 102215 | A6 | A6 | L9-B3 |
| 102216 | A6 | A6 | L9-B4 |
| 102217 | A6 | A6 | L9-B5 |
| 102218 | A6 | A6 | L9-B6 |
| 102219 | A6 | A6 | L9-B7 |
| 102220 | A6 | A6 | L9-B8 |
| 102221 | A6 | A6 | L9-B9 |
| 102222 | A6 | A6 | L9-B10 |
| 102223 | A6 | A6 | L9-B11 |
| 102224 | A6 | A6 | L9-B12 |
| 102225 | A6 | A6 | L9-B13 |
| 102226 | A6 | A6 | L9-B14 |
| 102227 | A6 | A6 | L10-B1 |
| 102228 | A6 | A6 | L10-B2 |
| 102229 | A6 | A6 | L10-B3 |
| 102230 | A6 | A6 | L10-B4 |
| 102231 | A6 | A6 | L10-B5 |
| 102232 | A6 | A6 | L10-B6 |
| 102233 | A6 | A6 | L10-B7 |
| 102234 | A6 | A6 | L10-B8 |
| 102235 | A6 | A6 | L10-B9 |
| 102236 | A6 | A6 | L10-B10 |
| 102237 | A6 | A6 | L10-B11 |
| 102238 | A6 | A6 | L10-B12 |
| 102239 | A6 | A6 | L10-B13 |
| 102240 | A6 | A6 | L10-B14 |
| 102241 | A6 | A6 | L11-B1 |
| 102242 | A6 | A6 | L11-B2 |
| 102243 | A6 | A6 | L11-B3 |
| 102244 | A6 | A6 | L11-B4 |
| 102245 | A6 | A6 | L11-B5 |
| 102246 | A6 | A6 | L11-B6 |
| 102247 | A6 | A6 | L11-B7 |
| 102248 | A6 | A6 | L11-B8 |
| 102249 | A6 | A6 | L11-B9 |
| 102250 | A6 | A6 | L11-B10 |
| 102251 | A6 | A6 | L11-B11 |
| 102252 | A6 | A6 | L11-B12 |
| 102253 | A6 | A6 | L11-B13 |
| 102254 | A6 | A6 | L11-B14 |
| 102255 | A6 | A6 | L12-B1 |
| 102256 | A6 | A6 | L12-B2 |
| 102257 | A6 | A6 | L12-B3 |
| 102258 | A6 | A6 | L12-B4 |
| 102259 | A6 | A6 | L12-B5 |
| 102260 | A6 | A6 | L12-B6 |
| 102261 | A6 | A6 | L12-B7 |
| 102262 | A6 | A6 | L12-B8 |
| 102263 | A6 | A6 | L12-B9 |
| 102264 | A6 | A6 | L12-B10 |
| 102265 | A6 | A6 | L12-B11 |
| 102266 | A6 | A6 | L12-B12 |
| 102267 | A6 | A6 | L12-B13 |
| 102268 | A6 | A6 | L12-B14 |
| 102269 | A6 | A6 | L13-B1 |
| 102270 | A6 | A6 | L13-B2 |
| 102271 | A6 | A6 | L13-B3 |
| 102272 | A6 | A6 | L13-B4 |
| 102273 | A6 | A6 | L13-B5 |
| 102274 | A6 | A6 | L13-B6 |
| 102275 | A6 | A6 | L13-B7 |
| 102276 | A6 | A6 | L13-B8 |
| 102277 | A6 | A6 | L13-B9 |
| 102278 | A6 | A6 | L13-B10 |
| 102279 | A6 | A6 | L13-B11 |
| 102280 | A6 | A6 | L13-B12 |
| 102281 | A6 | A6 | L13-B13 |
| 102282 | A6 | A6 | L13-B14 |
| 102283 | A6 | A6 | L14-B1 |
| 102284 | A6 | A6 | L14-B2 |
| 102285 | A6 | A6 | L14-B3 |
| 102286 | A6 | A6 | L14-B4 |
| 102287 | A6 | A6 | L14-B5 |
| 102288 | A6 | A6 | L14-B6 |
| 102289 | A6 | A6 | L14-B7 |
| 102290 | A6 | A6 | L14-B8 |
| 102291 | A6 | A6 | L14-B9 |
| 102292 | A6 | A6 | L14-B10 |
| 102293 | A6 | A6 | L14-B11 |
| 102294 | A6 | A6 | L14-B12 |
| 102295 | A6 | A6 | L14-B13 |
| 102296 | A6 | A6 | L14-B14 |
| 102297 | A6 | A6 | L15-B1 |
| 102298 | A6 | A6 | L15-B2 |
| 102299 | A6 | A6 | L15-B3 |
| 102300 | A6 | A6 | L15-B4 |
| 102301 | A6 | A6 | L15-B5 |
| 102302 | A6 | A6 | L15-B6 |
| 102303 | A6 | A6 | L15-B7 |
| 102304 | A6 | A6 | L15-B8 |
| 102305 | A6 | A6 | L15-B9 |
| 102306 | A6 | A6 | L15-B10 |
| 102307 | A6 | A6 | L15-B11 |
| 102308 | A6 | A6 | L15-B12 |
| 102309 | A6 | A6 | L15-B13 |
| 102310 | A6 | A6 | L15-B14 |
| 102311 | A6 | L1-B1 | L1-B1 |
| 102312 | A6 | L1-B2 | L1-B2 |
| 102313 | A6 | L1-B3 | L1-B3 |
| 102314 | A6 | L1-B4 | L1-B4 |
| 102315 | A6 | L1-B5 | L1-B5 |
| 102316 | A6 | L1-B6 | L1-B6 |
| 102317 | A6 | L1-B7 | L1-B7 |
| 102318 | A6 | L1-B8 | L1-B8 |
| 102319 | A6 | L1-B9 | L1-B9 |
| 102320 | A6 | L1-B10 | L1-B10 |
| 102321 | A6 | L1-B11 | L1-B11 |
| 102322 | A6 | L1-B12 | L1-B12 |
| 102323 | A6 | L1-B13 | L1-B13 |
| 102324 | A6 | L1-B14 | L1-B14 |
| 102325 | A6 | L2-B1 | L2-B1 |
| 102326 | A6 | L2-B2 | L2-B2 |
| 102327 | A6 | L2-B3 | L2-B3 |
| 102328 | A6 | L2-B4 | L2-B4 |
| 102329 | A6 | L2-B5 | L2-B5 |
| 102330 | A6 | L2-B6 | L2-B6 |
| 102331 | A6 | L2-B7 | L2-B7 |
| 102332 | A6 | L2-B8 | L2-B8 |
| 102333 | A6 | L2-B9 | L2-B9 |
| 102334 | A6 | L2-B10 | L2-B10 |
| 102335 | A6 | L2-B11 | L2-B11 |
| 102336 | A6 | L2-B12 | L2-B12 |
| 102337 | A6 | L2-B13 | L2-B13 |
| 102338 | A6 | L2-B14 | L2-B14 |
| 102339 | A6 | L3-B1 | L3-B1 |
| 102340 | A6 | L3-B2 | L3-B2 |
| 102341 | A6 | L3-B3 | L3-B3 |
| 102342 | A6 | L3-B4 | L3-B4 |
| 102343 | A6 | L3-B5 | L3-B5 |
| 102344 | A6 | L3-B6 | L3-B6 |
| 102345 | A6 | L3-B7 | L3-B7 |
| 102346 | A6 | L3-B8 | L3-B8 |
| 102347 | A6 | L3-B9 | L3-B9 |
| 102348 | A6 | L3-B10 | L3-B10 |
| 102349 | A6 | L3-B11 | L3-B11 |
| 102350 | A6 | L3-B12 | L3-B12 |
| 102351 | A6 | L3-B13 | L3-B13 |
| 102352 | A6 | L3-B14 | L3-B14 |
| 102353 | A6 | L4-B1 | L4-B1 |
| 102354 | A6 | L4-B2 | L4-B2 |
| 102355 | A6 | L4-B3 | L4-B3 |
| 102356 | A6 | L4-B4 | L4-B4 |
| 102357 | A6 | L4-B5 | L4-B5 |
| 102358 | A6 | L4-B6 | L4-B6 |
| 102359 | A6 | L4-B7 | L4-B7 |
| 102360 | A6 | L4-B8 | L4-B8 |
| 102361 | A6 | L4-B9 | L4-B9 |
| 102362 | A6 | L4-B10 | L4-B10 |
| 102363 | A6 | L4-B11 | L4-B11 |
| 102364 | A6 | L4-B12 | L4-B12 |
| 102365 | A6 | L4-B13 | L4-B13 |
| 102366 | A6 | L4-B14 | L4-B14 |
| 102367 | A6 | L5-B1 | L5-B1 |
| 102368 | A6 | L5-B2 | L5-B2 |
| 102369 | A6 | L5-B3 | L5-B3 |
| 102370 | A6 | L5-B4 | L5-B4 |
| 102371 | A6 | L5-B5 | L5-B5 |
| 102372 | A6 | L5-B6 | L5-B6 |
| 102373 | A6 | L5-B7 | L5-B7 |
| 102374 | A6 | L5-B8 | L5-B8 |
| 102375 | A6 | L5-B9 | L5-B9 |
| 102376 | A6 | L5-B10 | L5-B10 |
| 102377 | A6 | L5-B11 | L5-B11 |
| 102378 | A6 | L5-B12 | L5-B12 |
| 102379 | A6 | L5-B13 | L5-B13 |
| 102380 | A6 | L5-B14 | L5-B14 |
| 102381 | A6 | L6-B1 | L6-B1 |
| 102382 | A6 | L6-B2 | L6-B2 |
| 102383 | A6 | L6-B3 | L6-B3 |
| 102384 | A6 | L6-B4 | L6-B4 |
| 102385 | A6 | L6-B5 | L6-B5 |
| 102386 | A6 | L6-B6 | L6-B6 |
| 102387 | A6 | L6-B7 | L6-B7 |
| 102388 | A6 | L6-B8 | L6-B8 |
| 102389 | A6 | L6-B9 | L6-B9 |
| 102390 | A6 | L6-B10 | L6-B10 |
| 102391 | A6 | L6-B11 | L6-B11 |
| 102392 | A6 | L6-B12 | L6-B12 |
| 102393 | A6 | L6-B13 | L6-B13 |
| 102394 | A6 | L6-B14 | L6-B14 |
| 102395 | A6 | L7-B1 | L7-B1 |
| 102396 | A6 | L7-B2 | L7-B2 |
| 102397 | A6 | L7-B3 | L7-B3 |
| 102398 | A6 | L7-B4 | L7-B4 |
| 102399 | A6 | L7-B5 | L7-B5 |
| 102400 | A6 | L7-B6 | L7-B6 |
| 102401 | A6 | L7-B7 | L7-B7 |
| 102402 | A6 | L7-B8 | L7-B8 |
| 102403 | A6 | L7-B9 | L7-B9 |
| 102404 | A6 | L7-B10 | L7-B10 |
| 102405 | A6 | L7-B11 | L7-B11 |
| 102406 | A6 | L7-B12 | L7-B12 |
| 102407 | A6 | L7-B13 | L7-B13 |
| 102408 | A6 | L7-B14 | L7-B14 |
| 102409 | A6 | L8-B1 | L8-B1 |
| 102410 | A6 | L8-B2 | L8-B2 |
| 102411 | A6 | L8-B3 | L8-B3 |
| 102412 | A6 | L8-B4 | L8-B4 |
| 102413 | A6 | L8-B5 | L8-B5 |
| 102414 | A6 | L8-B6 | L8-B6 |
| 102415 | A6 | L8-B7 | L8-B7 |
| 102416 | A6 | L8-B8 | L8-B8 |
| 102417 | A6 | L8-B9 | L8-B9 |
| 102418 | A6 | L8-B10 | L8-B10 |
| 102419 | A6 | L8-B11 | L8-B11 |
| 102420 | A6 | L8-B12 | L8-B12 |
| 102421 | A6 | L8-B13 | L8-B13 |
| 102422 | A6 | L8-B14 | L8-B14 |
| 102423 | A6 | L9-B1 | L9-B1 |
| 102424 | A6 | L9-B2 | L9-B2 |
| 102425 | A6 | L9-B3 | L9-B3 |
| 102426 | A6 | L9-B4 | L9-B4 |
| 102427 | A6 | L9-B5 | L9-B5 |
| 102428 | A6 | L9-B6 | L9-B6 |
| 102429 | A6 | L9-B7 | L9-B7 |
| 102430 | A6 | L9-B8 | L9-B8 |
| 102431 | A6 | L9-B9 | L9-B9 |
| 102432 | A6 | L9-B10 | L9-B10 |
| 102433 | A6 | L9-B11 | L9-B11 |
| 102434 | A6 | L9-B12 | L9-B12 |
| 102435 | A6 | L9-B13 | L9-B13 |
| 102436 | A6 | L9-B14 | L9-B14 |
| 102437 | A6 | L10-B1 | L10-B1 |
| 102438 | A6 | L10-B2 | L10-B2 |
| 102439 | A6 | L10-B3 | L10-B3 |
| 102440 | A6 | L10-B4 | L10-B4 |
| 102441 | A6 | L10-B5 | L10-B5 |
| 102442 | A6 | L10-B6 | L10-B6 |
| 102443 | A6 | L10-B7 | L10-B7 |
| 102444 | A6 | L10-B8 | L10-B8 |
| 102445 | A6 | L10-B9 | L10-B9 |
| 102446 | A6 | L10-B10 | L10-B10 |
| 102447 | A6 | L10-B11 | L10-B11 |
| 102448 | A6 | L10-B12 | L10-B12 |
| 102449 | A6 | L10-B13 | L10-B13 |
| 102450 | A6 | L10-B14 | L10-B14 |
| 102451 | A6 | L11-B1 | L11-B1 |
| 102452 | A6 | L11-B2 | L11-B2 |
| 102453 | A6 | L11-B3 | L11-B3 |
| 102454 | A6 | L11-B4 | L11-B4 |
| 102455 | A6 | L11-B5 | L11-B5 |
| 102456 | A6 | L11-B6 | L11-B6 |
| 102457 | A6 | L11-B7 | L11-B7 |
| 102458 | A6 | L11-B8 | L11-B8 |
| 102459 | A6 | L11-B9 | L11-B9 |
| 102460 | A6 | L11-B10 | L11-B10 |
| 102461 | A6 | L11-B11 | L11-B11 |
| 102462 | A6 | L11-B12 | L11-B12 |
| 102463 | A6 | L11-B13 | L11-B13 |
| 102464 | A6 | L11-B14 | L11-B14 |
| 102465 | A6 | L12-B1 | L12-B1 |
| 102466 | A6 | L12-B2 | L12-B2 |
| 102467 | A6 | L12-B3 | L12-B3 |
| 102468 | A6 | L12-B4 | L12-B4 |
| 102469 | A6 | L12-B5 | L12-B5 |
| 102470 | A6 | L12-B6 | L12-B6 |
| 102471 | A6 | L12-B7 | L12-B7 |
| 102472 | A6 | L12-B8 | L12-B8 |
| 102473 | A6 | L12-B9 | L12-B9 |
| 102474 | A6 | L12-B10 | L12-B10 |
| 102475 | A6 | L12-B11 | L12-B11 |
| 102476 | A6 | L12-B12 | L12-B12 |
| 102477 | A6 | L12-B13 | L12-B13 |
| 102478 | A6 | L12-B14 | L12-B14 |
| 102479 | A6 | L13-B1 | L13-B1 |
| 102480 | A6 | L13-B2 | L13-B2 |
| 102481 | A6 | L13-B3 | L13-B3 |
| 102482 | A6 | L13-B4 | L13-B4 |
| 102483 | A6 | L13-B5 | L13-B5 |
| 102484 | A6 | L13-B6 | L13-B6 |
| 102485 | A6 | L13-B7 | L13-B7 |
| 102486 | A6 | L13-B8 | L13-B8 |
| 102487 | A6 | L13-B9 | L13-B9 |
| 102488 | A6 | L13-B10 | L13-B10 |
| 102489 | A6 | L13-B11 | L13-B11 |
| 102490 | A6 | L13-B12 | L13-B12 |
| 102491 | A6 | L13-B13 | L13-B13 |
| 102492 | A6 | L13-B14 | L13-B14 |
| 102493 | A6 | L14-B1 | L14-B1 |
| 102494 | A6 | L14-B2 | L14-B2 |
| 102495 | A6 | L14-B3 | L14-B3 |
| 102496 | A6 | L14-B4 | L14-B4 |
| 102497 | A6 | L14-B5 | L14-B5 |
| 102498 | A6 | L14-B6 | L14-B6 |
| 102499 | A6 | L14-B7 | L14-B7 |
| 102500 | A6 | L14-B8 | L14-B8 |
| 102501 | A6 | L14-B9 | L14-B9 |
| 102502 | A6 | L14-B10 | L14-B10 |
| 102503 | A6 | L14-B11 | L14-B11 |
| 102504 | A6 | L14-B12 | L14-B12 |
| 102505 | A6 | L14-B13 | L14-B13 |
| 102506 | A6 | L14-B14 | L14-B14 |
| 102507 | A6 | L15-B1 | L15-B1 |
| 102508 | A6 | L15-B2 | L15-B2 |
| 102509 | A6 | L15-B3 | L15-B3 |
| 102510 | A6 | L15-B4 | L15-B4 |
| 102511 | A6 | L15-B5 | L15-B5 |
| 102512 | A6 | L15-B6 | L15-B6 |
| 102513 | A6 | L15-B7 | L15-B7 |
| 102514 | A6 | L15-B8 | L15-B8 |
| 102515 | A6 | L15-B9 | L15-B9 |
| 102516 | A6 | L15-B10 | L15-B10 |
| 102517 | A6 | L15-B11 | L15-B11 |
| 102518 | A6 | L15-B12 | L15-B12 |
| 102519 | A6 | L15-B13 | L15-B13 |
| 102520 | A6 | L15-B14 | L15-B14 |
| 102521 | L1-B1 | L1-B1 | L1-B1 |
| 102522 | L1-B2 | L1-B2 | L1-B2 |
| 102523 | L1-B3 | L1-B3 | L1-B3 |
| 102524 | L1-B4 | L1-B4 | L1-B4 |
| 102525 | L1-B5 | L1-B5 | L1-B5 |
| 102526 | L1-B6 | L1-B6 | L1-B6 |
| 102527 | L1-B7 | L1-B7 | L1-B7 |
| 102528 | L1-B8 | L1-B8 | L1-B8 |
| 102529 | L1-B9 | L1-B9 | L1-B9 |
| 102530 | L1-B10 | L1-B10 | L1-B10 |
| 102531 | L1-B11 | L1-B11 | L1-B11 |
| 102532 | L1-B12 | L1-B12 | L1-B12 |
| 102533 | L1-B13 | L1-B13 | L1-B13 |
| 102534 | L1-B14 | L1-B14 | L1-B14 |
| 102535 | L2-B1 | L2-B1 | L2-B1 |
| 102536 | L2-B2 | L2-B2 | L2-B2 |
| 102537 | L2-B3 | L2-B3 | L2-B3 |
| 102538 | L2-B4 | L2-B4 | L2-B4 |
| 102539 | L2-B5 | L2-B5 | L2-B5 |
| 102540 | L2-B6 | L2-B6 | L2-B6 |
| 102541 | L2-B7 | L2-B7 | L2-B7 |
| 102542 | L2-B8 | L2-B8 | L2-B8 |
| 102543 | L2-B9 | L2-B9 | L2-B9 |
| 102544 | L2-B10 | L2-B10 | L2-B10 |
| 102545 | L2-B11 | L2-B11 | L2-B11 |
| 102546 | L2-B12 | L2-B12 | L2-B12 |
| 102547 | L2-B13 | L2-B13 | L2-B13 |
| 102548 | L2-B14 | L2-B14 | L2-B14 |
| 102549 | L3-B1 | L3-B1 | L3-B1 |
| 102550 | L3-B2 | L3-B2 | L3-B2 |
| 102551 | L3-B3 | L3-B3 | L3-B3 |
| 102552 | L3-B4 | L3-B4 | L3-B4 |
| 102553 | L3-B5 | L3-B5 | L3-B5 |
| 102554 | L3-B6 | L3-B6 | L3-B6 |
| 102555 | L3-B7 | L3-B7 | L3-B7 |
| 102556 | L3-B8 | L3-B8 | L3-B8 |
| 102557 | L3-B9 | L3-B9 | L3-B9 |
| 102558 | L3-B10 | L3-B10 | L3-B10 |
| 102559 | L3-B11 | L3-B11 | L3-B11 |
| 102560 | L3-B12 | L3-B12 | L3-B12 |
| 102561 | L3-B13 | L3-B13 | L3-B13 |
| 102562 | L3-B14 | L3-B14 | L3-B14 |
| 102563 | L4-B1 | L4-B1 | L4-B1 |
| 102564 | L4-B2 | L4-B2 | L4-B2 |
| 102565 | L4-B3 | L4-B3 | L4-B3 |
| 102566 | L4-B4 | L4-B4 | L4-B4 |
| 102567 | L4-B5 | L4-B5 | L4-B5 |
| 102568 | L4-B6 | L4-B6 | L4-B6 |
| 102569 | L4-B7 | L4-B7 | L4-B7 |
| 102570 | L4-B8 | L4-B8 | L4-B8 |
| 102571 | L4-B9 | L4-B9 | L4-B9 |
| 102572 | L4-B10 | L4-B10 | L4-B10 |
| 102573 | L4-B11 | L4-B11 | L4-B11 |
| 102574 | L4-B12 | L4-B12 | L4-B12 |
| 102575 | L4-B13 | L4-B13 | L4-B13 |
| 102576 | L4-B14 | L4-B14 | L4-B14 |
| 102577 | L5-B1 | L5-B1 | L5-B1 |
| 102578 | L5-B2 | L5-B2 | L5-B2 |
| 102579 | L5-B3 | L5-B3 | L5-B3 |
| 102580 | L5-B4 | L5-B4 | L5-B4 |
| 102581 | L5-B5 | L5-B5 | L5-B5 |
| 102582 | L5-B6 | L5-B6 | L5-B6 |
| 102583 | L5-B7 | L5-B7 | L5-B7 |
| 102584 | L5-B8 | L5-B8 | L5-B8 |
| 102585 | L5-B9 | L5-B9 | L5-B9 |
| 102586 | L5-B10 | L5-B10 | L5-B10 |
| 102587 | L5-B11 | L5-B11 | L5-B11 |
| 102588 | L5-B12 | L5-B12 | L5-B12 |
| 102589 | L5-B13 | L5-B13 | L5-B13 |
| 102590 | L5-B14 | L5-B14 | L5-B14 |
| 102591 | L6-B1 | L6-B1 | L6-B1 |
| 102592 | L6-B2 | L6-B2 | L6-B2 |
| 102593 | L6-B3 | L6-B3 | L6-B3 |
| 102594 | L6-B4 | L6-B4 | L6-B4 |
| 102595 | L6-B5 | L6-B5 | L6-B5 |
| 102596 | L6-B6 | L6-B6 | L6-B6 |
| 102597 | L6-B7 | L6-B7 | L6-B7 |
| 102598 | L6-B8 | L6-B8 | L6-B8 |
| 102599 | L6-B9 | L6-B9 | L6-B9 |
| 102600 | L6-B10 | L6-B10 | L6-B10 |
| 102601 | L6-B11 | L6-B11 | L6-B11 |
| 102602 | L6-B12 | L6-B12 | L6-B12 |
| 102603 | L6-B13 | L6-B13 | L6-B13 |
| 102604 | L6-B14 | L6-B14 | L6-B14 |
| 102605 | L7-B1 | L7-B1 | L7-B1 |
| 102606 | L7-B2 | L7-B2 | L7-B2 |
| 102607 | L7-B3 | L7-B3 | L7-B3 |
| 102608 | L7-B4 | L7-B4 | L7-B4 |
| 102609 | L7-B5 | L7-B5 | L7-B5 |
| 102610 | L7-B6 | L7-B6 | L7-B6 |
| 102611 | L7-B7 | L7-B7 | L7-B7 |
| 102612 | L7-B8 | L7-B8 | L7-B8 |
| 102613 | L7-B9 | L7-B9 | L7-B9 |
| 102614 | L7-B10 | L7-B10 | L7-B10 |
| 102615 | L7-B11 | L7-B11 | L7-B11 |
| 102616 | L7-B12 | L7-B12 | L7-B12 |
| 102617 | L7-B13 | L7-B13 | L7-B13 |
| 102618 | L7-B14 | L7-B14 | L7-B14 |
| 102619 | L8-B1 | L8-B1 | L8-B1 |
| 102620 | L8-B2 | L8-B2 | L8-B2 |
| 102621 | L8-B3 | L8-B3 | L8-B3 |
| 102622 | L8-B4 | L8-B4 | L8-B4 |
| 102623 | L8-B5 | L8-B5 | L8-B5 |
| 102624 | L8-B6 | L8-B6 | L8-B6 |
| 102625 | L8-B7 | L8-B7 | L8-B7 |
| 102626 | L8-B8 | L8-B8 | L8-B8 |
| 102627 | L8-B9 | L8-B9 | L8-B9 |
| 102628 | L8-B10 | L8-B10 | L8-B10 |
| 102629 | L8-B11 | L8-B11 | L8-B11 |
| 102630 | L8-B12 | L8-B12 | L8-B12 |
| 102631 | L8-B13 | L8-B13 | L8-B13 |
| 102632 | L8-B14 | L8-B14 | L8-B14 |
| 102633 | L9-B1 | L9-B1 | L9-B1 |
| 102634 | L9-B2 | L9-B2 | L9-B2 |
| 102635 | L9-B3 | L9-B3 | L9-B3 |
| 102636 | L9-B4 | L9-B4 | L9-B4 |
| 102637 | L9-B5 | L9-B5 | L9-B5 |
| 102638 | L9-B6 | L9-B6 | L9-B6 |
| 102639 | L9-B7 | L9-B7 | L9-B7 |
| 102640 | L9-B8 | L9-B8 | L9-B8 |
| 102641 | L9-B9 | L9-B9 | L9-B9 |
| 102642 | L9-B10 | L9-B10 | L9-B10 |
| 102643 | L9-B11 | L9-B11 | L9-B11 |
| 102644 | L9-B12 | L9-B12 | L9-B12 |
| 102645 | L9-B13 | L9-B13 | L9-B13 |
| 102646 | L9-B14 | L9-B14 | L9-B14 |
| 102647 | L10-B1 | L10-B1 | L10-B1 |
| 102648 | L10-B2 | L10-B2 | L10-B2 |
| 102649 | L10-B3 | L10-B3 | L10-B3 |
| 102650 | L10-B4 | L10-B4 | L10-B4 |
| 102651 | L10-B5 | L10-B5 | L10-B5 |
| 102652 | L10-B6 | L10-B6 | L10-B6 |
| 102653 | L10-B7 | L10-B7 | L10-B7 |
| 102654 | L10-B8 | L10-B8 | L10-B8 |
| 102655 | L10-B9 | L10-B9 | L10-B9 |
| 102656 | L10-B10 | L10-B10 | L10-B10 |
| 102657 | L10-B11 | L10-B11 | L10-B11 |
| 102658 | L10-B12 | L10-B12 | L10-B12 |
| 102659 | L10-B13 | L10-B13 | L10-B13 |
| 102660 | L10-B14 | L10-B14 | L10-B14 |
| 102661 | L11-B1 | L11-B1 | L11-B1 |
| 102662 | L11-B2 | L11-B2 | L11-B2 |
| 102663 | L11-B3 | L11-B3 | L11-B3 |
| 102664 | L11-B4 | L11-B4 | L11-B4 |
| 102665 | L11-B5 | L11-B5 | L11-B5 |
| 102666 | L11-B6 | L11-B6 | L11-B6 |
| 102667 | L11-B7 | L11-B7 | L11-B7 |
| 102668 | L11-B8 | L11-B8 | L11-B8 |
| 102669 | L11-B9 | L11-B9 | L11-B9 |
| 102670 | L11-B10 | L11-B10 | L11-B10 |
| 102671 | L11-B11 | L11-B11 | L11-B11 |
| 102672 | L11-B12 | L11-B12 | L11-B12 |
| 102673 | L11-B13 | L11-B13 | L11-B13 |
| 102674 | L11-B14 | L11-B14 | L11-B14 |
| 102675 | L12-B1 | L12-B1 | L12-B1 |
| 102676 | L12-B2 | L12-B2 | L12-B2 |
| 102677 | L12-B3 | L12-B3 | L12-B3 |
| 102678 | L12-B4 | L12-B4 | L12-B4 |
| 102679 | L12-B5 | L12-B5 | L12-B5 |
| 102680 | L12-B6 | L12-B6 | L12-B6 |
| 102681 | L12-B7 | L12-B7 | L12-B7 |
| 102682 | L12-B8 | L12-B8 | L12-B8 |
| 102683 | L12-B9 | L12-B9 | L12-B9 |
| 102684 | L12-B10 | L12-B10 | L12-B10 |
| 102685 | L12-B11 | L12-B11 | L12-B11 |
| 102686 | L12-B12 | L12-B12 | L12-B12 |
| 102687 | L12-B13 | L12-B13 | L12-B13 |
| 102688 | L12-B14 | L12-B14 | L12-B14 |
| 102689 | L13-B1 | L13-B1 | L13-B1 |
| 102690 | L13-B2 | L13-B2 | L13-B2 |
| 102691 | L13-B3 | L13-B3 | L13-B3 |
| 102692 | L13-B4 | L13-B4 | L13-B4 |
| 102693 | L13-B5 | L13-B5 | L13-B5 |
| 102694 | L13-B6 | L13-B6 | L13-B6 |
| 102695 | L13-B7 | L13-B7 | L13-B7 |
| 102696 | L13-B8 | L13-B8 | L13-B8 |
| 102697 | L13-B9 | L13-B9 | L13-B9 |
| 102698 | L13-B10 | L13-B10 | L13-B10 |
| 102699 | L13-B11 | L13-B11 | L13-B11 |
| 102700 | L13-B12 | L13-B12 | L13-B12 |
| 102701 | L13-B13 | L13-B13 | L13-B13 |
| 102702 | L13-B14 | L13-B14 | L13-B14 |
| 102703 | L14-B1 | L14-B1 | L14-B1 |
| 102704 | L14-B2 | L14-B2 | L14-B2 |
| 102705 | L14-B3 | L14-B3 | L14-B3 |
| 102706 | L14-B4 | L14-B4 | L14-B4 |
| 102707 | L14-B5 | L14-B5 | L14-B5 |
| 102708 | L14-B6 | L14-B6 | L14-B6 |
| 102709 | L14-B7 | L14-B7 | L14-B7 |
| 102710 | L14-B8 | L14-B8 | L14-B8 |
| 102711 | L14-B9 | L14-B9 | L14-B9 |
| 102712 | L14-B10 | L14-B10 | L14-B10 |
| 102713 | L14-B11 | L14-B11 | L14-B11 |
| 102714 | L14-B12 | L14-B12 | L14-B12 |
| 102715 | L14-B13 | L14-B13 | L14-B13 |
| 102716 | L14-B14 | L14-B14 | L14-B14 |
| 102717 | L15-B1 | L15-B1 | L15-B1 |
| 102718 | L15-B2 | L15-B2 | L15-B2 |
| 102719 | L15-B3 | L15-B3 | L15-B3 |
| 102720 | L15-B4 | L15-B4 | L15-B4 |
| 102721 | L15-B5 | L15-B5 | L15-B5 |
| 102722 | L15-B6 | L15-B6 | L15-B6 |
| 102723 | L15-B7 | L15-B7 | L15-B7 |
| 102724 | L15-B8 | L15-B8 | L15-B8 |
| 102725 | L15-B9 | L15-B9 | L15-B9 |
| 102726 | L15-B10 | L15-B10 | L15-B10 |
| 102727 | L15-B11 | L15-B11 | L15-B11 |
| 102728 | L15-B12 | L15-B12 | L15-B12 |
| 102729 | L15-B13 | L15-B13 | L15-B13 |
| 102730 | L15-B14 | L15-B14 | L15-B14 |

The molecular weight of the compound represented by the general formula (1) is, for example, when the compound is intended to be used in an organic layer to be formed through vapor deposition, preferably 1500 or less, more preferably 1200 or less, even more preferably 1000 or less, and especially more preferably 900 or less. The lower limit of the molecular weight is the molecular weight of the smallest compound represented by the general formula (1).

A film of the compound represented by the general formula (1) may be formed according to a coating method irrespective of the molecular weight thereof. According to a coating method, a film of the compound having a relatively large molecular weight can be formed.

Applying the present invention, use of a compound containing plural structures represented by the general formula (1) in the molecule as a host material may be taken into consideration.

For example, a polymerizable group is previously introduced into a structure represented by the general formula (1), and the polymerizable group may be polymerized to give a polymer, and the resultant polymer may be used as a light emitting material. Specifically, a monomer having a polymerizable functional group at any of Ar¹ to Ar³ and R¹ to R⁸ of the general formula (1) is prepared, and this is polymerized singly or is copolymerized with any other polymer to give a polymer having a recurring unit of the monomer, and the resultant polymer may be used as a light emitting material. Alternatively, compounds each having a structure represented by the general formula (1) are coupled to give a dimer or a trimer, which may be used as a light emitting material.

Examples of the polymer having a recurring unit containing a structure represented by the general formula (1) include polymers having a structure represented by the following general formula (11) or (12).

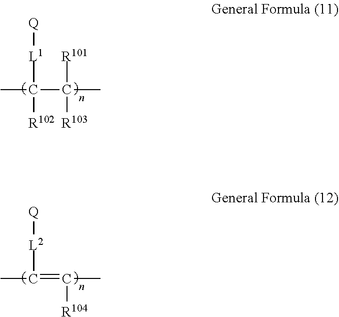

In the general formula (11) or (12), Q represents a group containing a structure represented by the general formula (1), and L¹ and L² each represent a linking group. The carbon number of the linking group is preferably 0 to 20, more preferably 1 to 15, even more preferably 2 to 10. The linking group preferably has a structure represented by —X¹¹-L¹¹-. In this, X¹¹ represents an oxygen atom or a sulfur atom, and is preferably an oxygen atom. L¹¹ represents a linking group, and is preferably a substituted or unsubstituted alkylene group, or a substituted or unsubstituted arylene group, and is more preferably a substituted or unsubstituted alkylene group having 1 to 10 carbon atoms, or a substituted or unsubstituted phenylene group.

In the general formula (11) or (12), R¹⁰¹, R¹⁰², R¹⁰³ and R¹⁰⁴ each independently represent a substituent. Preferably, the substituent is a substituted or unsubstituted alkyl group having 1 to 6 carbon atoms, a substituted or unsubstituted alkoxy group having 1 to 6 carbon atoms, or a halogen atom, more preferably an unsubstituted alkyl group having 1 to 3 carbon atoms, an unsubstituted alkoxy group having 1 to 3 carbon atoms, a fluorine atom or a chlorine atom, and even more preferably an unsubstituted alkyl group having 1 to 3 carbon atoms, or an unsubstituted alkoxy group having 1 to 3 carbon atoms.

The linking group represented by L¹ and L² may bond to any of Ar¹ to Ar³, and R¹ to R⁸ in the structure of the general formula (1) that constitutes Q. Two or more such linking groups may bond to one Q to form a crosslinked structure or a network structure.

Specific structural examples of the recurring units includes structures represented by the following formulae (13) to (16).

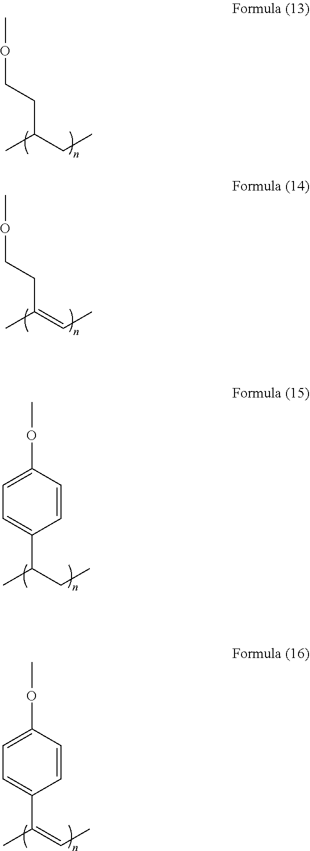

The polymer having a recurring unit containing any of these formulae (13) to (16) may be produced by previously introducing a hydroxyl group in any of Ar¹ to Ar³ and R¹ to R⁸ in the structure of the general formula (1), then introducing a polymerizable group into the hydroxyl group serving as a linker through reaction with any of the following compounds, and polymerizing the polymerizable group to give the polymer.

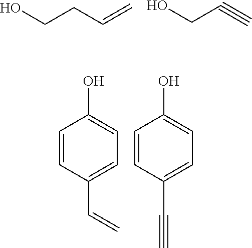

The polymer containing a structure represented by the general formula (1) in the molecule may be a polymer composed of a recurring unit alone having a structure represented by the general formula (1), or may be a polymer additionally containing a recurring unit having any other structure. One kind alone or two or more kinds of recurring units having a structure represented by the general formula (1) may be contained in the polymer. The other recurring unit not having a structure represented by the general formula (1) includes those derived from monomers to be used in ordinary copolymerization. Examples thereof include recurring units derived from monomers having an ethylenic unsaturated bond, such as ethylene and styrene.

### [Method for Synthesis of Compounds Represented by General Formula (1)]

The compounds represented by the general formula (1) are novel compounds.

The compounds represented by the general formula (1) may be synthesized by combination of known reactions. For example, a compound where Ar¹ and Ar² each are a phenyl group substituted with a group containing a skeleton represented by the general formula (2) and where the group containing a skeleton represented by the general formula (2) bonds to the meta-position of the phenyl group relative to the bonding position to the triazine ring, at the bonding position of R¹ via a single bond therebetween may be synthesized through reaction shown by the following reaction formula 1 or 2.

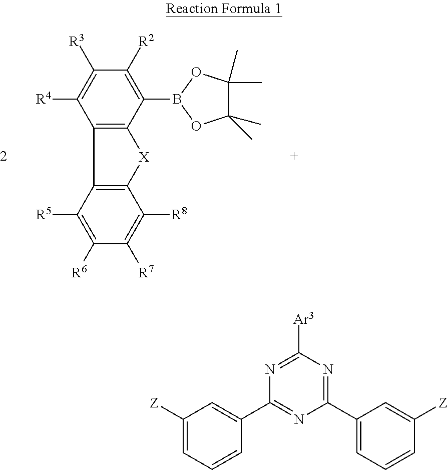

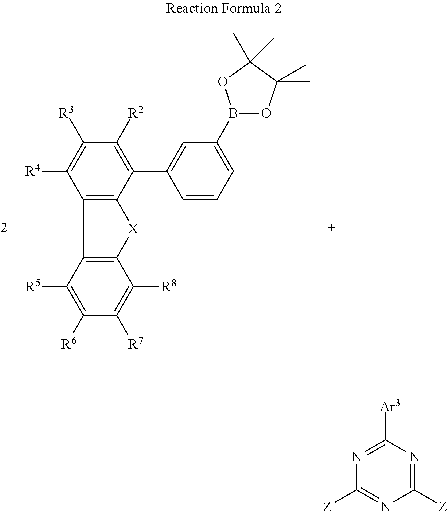

Regarding the description of Ar³, X, and R² to R⁸ in the above-mentioned reaction formulae, reference may be made to the corresponding description in the general formula (1). Z each independently represents a halogen atom, including a fluorine atom, a chlorine atom, a bromine atom and an iodine atom, and is preferably a bromine atom.

The above-mentioned reaction utilizes known coupling reaction, and can be carried out by suitably selecting known reaction conditions. Regarding the details of the above-mentioned reaction, reference may be made to Synthesis Examples given hereinunder. In addition, the compounds represented by the general formula (1) may also be synthesized by combination of any other known synthesis reactions.

### [Organic Light Emitting Device]

The compounds represented by the general formula (1) of the present invention include compounds useful as a host material for organic light emitting devices. Such compounds represented by the general formula (1) of the present invention can be effectively used as a host material in the light emitting layer of an organic light emitting device. In addition, the compounds represented by the general formula (1) of the present invention may also be used as a light emitting material (especially as a delayed fluorescent material) or an assist dopant, and further as an electron transport material or a hole transport material, or a hole blocking material or an electron blocking material. Here, the “host material” in the present invention is an organic compound contained in a light emitting layer in an amount larger than that of the light emitting material therein, and is an organic compound having a highest, lowest excited singlet state energy level among the organic compounds contained in the light emitting layer. The “assist dopant” is an organic compound which, in a light emitting layer containing at least the assist dopant, a host and a light emitting material, so acts that the light emission efficiency of the light emitting material therein can be higher than that of the light emitting material in a light emitting layer having the same composition as that of the light emitting layer but not containing an assist dopant.

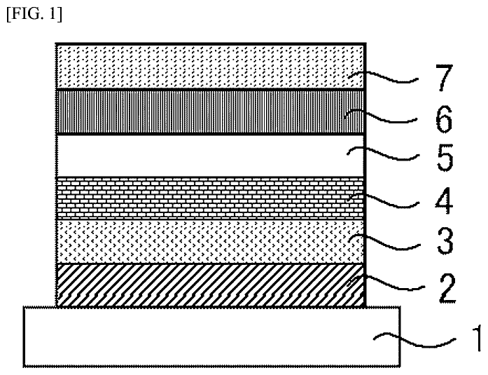

Using the compound represented by the general formula (1) of the present invention as a host material in a light emitting layer, an excellent organic light emitting device such as an excellent organic photoluminescent device (organic PL device) or organic electroluminescent device (organic EL device) can be provided. An organic photoluminescent device has a structure having at least a light emitting layer formed on a substrate. An organic electroluminescent device has a structure having at least an anode, a cathode and an organic layer formed between the anode and the cathode. The organic layer contains at least a light emitting layer, and may be formed of a light emitting layer alone, or may have one or more other organic layers than the light emitting layer. Such other organic layers include a hole transport layer, a hole injection layer, an electron blocking layer, a hole blocking layer, an electron injection layer, an electron transport layer, and an exciton blocking layer. The hole transport layer may be a hole injection transport layer having a hole injection function, and the electron transport layer may be an electron injection transport layer having an electron injection function. A specific example of a structure of an organic electroluminescent device is shown in FIG. 1. In FIG. 1, 1 is a substrate, 2 is an anode, 3 is a hole injection layer, 4 is a hole transport layer, 5 is a light emitting layer, 6 is an electron transport layer, and 7 is a cathode.

In the following, the members and the layers of the organic electroluminescent device are described. The description of the substrate and the light emitting layer may apply also to that of the substrate and the light emitting layer of an organic photoluminescent device.

### (Substrate)

Preferably, the organic electroluminescent device of the present invention is supported by a substrate. With no specific limitation, the substrate may be any one generally used in already existing organic electroluminescent devices, and for example, those formed of glass, transparent plastics, quartz or silicon may be used here.

### (Anode)

The anode of the organic electroluminescent device used is preferably formed of as an electrode material a metal, an alloy or an electroconductive compound each having a large work function (4 eV or more), or a mixture thereof. Specific examples of the electrode material include a metal, such as Au, and an electroconductive transparent material, such as CuI, indium tin oxide (ITO), SnO₂ and ZnO. A material that is amorphous and is capable of forming a transparent electroconductive film, such as IDIXO (In₂O₃—ZnO), may also be used. The anode may be formed in such a manner that the electrode material is formed into a thin film by such a method as vapor deposition or sputtering, and the film is patterned into a desired pattern by a photolithography method, or in the case where the pattern may not require high accuracy (for example, approximately 100 μm or more), the pattern may be formed with a mask having a desired shape on vapor deposition or sputtering of the electrode material. In alternative, in the case where a material capable of being applied as a coating, such as an organic electroconductive compound, is used, a wet film forming method, such as a printing method and a coating method, may be used. In the case where emitted light is to be taken out through the anode, the anode preferably has a transmittance of more than 10%, and the anode preferably has a sheet resistance of several hundred Ohm per square or less. The thickness thereof may be generally selected from a range of from 10 to 1,000 nm, and preferably from 10 to 200 nm, while depending on the material used.

### (Cathode)

The cathode is preferably formed of as an electrode material a metal having a small work function (4 eV or less) (referred to as an electron injection metal), an alloy or an electroconductive compound each having a small work function (4 eV or less), or a mixture thereof. Specific examples of the electrode material include sodium, a sodium-potassium alloy, magnesium, lithium, a magnesium-cupper mixture, a magnesium-silver mixture, a magnesium-aluminum mixture, a magnesium-indium mixture, an aluminum-aluminum oxide (Al₂O₃) mixture, indium, a lithium-aluminum mixture, and a rare earth metal. Among these, a mixture of an electron injection metal and a second metal that is a stable metal having a larger work function than the electron injection metal, for example, a magnesium-silver mixture, a magnesium-aluminum mixture, a magnesium-indium mixture, an aluminum-aluminum oxide (Al₂O₃) mixture, a lithium-aluminum mixture, and aluminum, are preferred from the standpoint of the electron injection property and the durability against oxidation and the like. The cathode may be produced by forming the electrode material into a thin film by such a method as vapor deposition or sputtering. The cathode preferably has a sheet resistance of several hundred Ohm per square or less, and the thickness thereof may be generally selected from a range of from 10 nm to 5 μm, and preferably from 50 to 200 nm. For transmitting the emitted light, any one of the anode and the cathode of the organic electroluminescent device is preferably transparent or translucent, thereby enhancing the light emission luminance.

The cathode may be formed with the electroconductive transparent materials described for the anode, thereby forming a transparent or translucent cathode, and by applying the cathode, a device having an anode and a cathode, both of which have transmittance, may be produced.

### (Light Emitting Layer)

The light emitting layer is a layer in which holes and electrons injected from an anode and a cathode are recombined to give excitons for light emission, and contains at least a light emitting material and a host material.

The light emitting material contained in the light emitting layer may be a fluorescent light emitting material or a phosphorescent light emitting material. Also the light emitting material may be a delayed fluorescent material that emits delayed fluorescence along with ordinary fluorescence. Delayed fluorescence is a fluorescent light to be emitted by a compound that has been in an excited state as given energy, in such a manner that the compound undergoes reverse intersystem crossing from the excited triplet state to an excited singlet state and thereafter returns back from the excited single state to a ground state, and is a fluorescent light that is observed later from the fluorescence from the directly occurring excited singlet state (ordinary fluorescent light). Using a light emitting material that emits such a delayed fluorescent light, a high light emission efficiency can be attained.

The host material is an organic compound having a highest, lowest excited single energy level among the organic compounds contained in the light emitting layer. The post material in the light emitting layer is preferably an organic compound having hole transportability and electron transportability, capable of preventing prolongation of the wavelength of the light emission and having a high glass transition temperature. In the present invention, one or more selected from the compound group of the compounds represented by the general formula (1) can be used. Here, the organic compounds contained in the light emitting layer at least include a light emitting material and a host material, and the other organic compound that may be in the light emitting layer is an assist dopant. When the light emitting layer contains a compound represented by the general formula (1) as a host material, the singlet-state exciton formed in the light emitting layer can be effectively confined in the molecule of the light emitting material and the energy thereof can be effectively used as an energy for light emission. As a result, an organic electroluminescence device having a high light emission efficiency can be realized. Also preferably, among the organic compounds to be contained in the light emitting layer, a compound having a highest, lowest excited singlet energy level and capable of having a highest, lowest excited triplet energy level is selected from the compound group represented by the general formula (1) and used as the host material. In that manner, along with the singlet state exciton formed in the light emitting material, the triplet state exciton can also be effectively confined in the molecule of the light emitting material, and the energy thereof can be effectively used for light emission.

In the organic electroluminescent device of the present invention, light emission occurs from the light emitting layer. The light emission may be any of fluorescent light emission, delayed fluorescent light emission or phosphorescent light emission, or may be a mixture thereof. The light emission may also be partly from a host material.

The lower limit of the content of the compound represented by the general formula (1) in the light emitting layer is, for example, more than 1% by weight, more than 5% by weight or more than 10% by weight. The upper limit is preferably less than 99.999% by weight, and may be, for example, less than 99.99% by weight, less than 99% by weight, less than 98% by weight, or less than 95% by weight. In the case where the compound represented by the general formula (1) is used as a host material, the content thereof in the light emitting layer is preferably more than 50% by weight, and is also preferably more than 70% by weight.

As described above, the light emitting material for use in the light emitting layer may be any of a fluorescent material, a phosphorescent material or a delayed fluorescent material, but from the viewpoint of attaining high light emission efficiency, a phosphorescent material or a delayed fluorescent material is preferred. The reason why a delayed fluorescent material can attain a high light emission efficiency is because of the following principle.

In an organic electroluminescent device, carriers are injected into the light emitting material from both positive and negative electrodes whereby the light emitting material is made to be in an excited state to emit light. In general, in the case of a carrier injection-type organic electroluminescent device, 25% of the formed excitons are made to be in an excited singlet state and the remaining 75% thereof are excited in an excited triplet state. Accordingly, phosphorescence emission from the excited triplet state enables a higher energy utilization efficiency. However, since the life of the excited triplet state is long, energy deactivation may occur owing to saturation of the excited state or the interaction of the exciton in an excited triplet state, and therefore the phosphorescence quantum efficiency is generally not so high in many cases. On the other hand, regarding the delayed fluorescent material, after energy transfer to the excited triplet state through intersystem crossing therein, reverse intersystem crossing to an excited single state occurs through triplet-triplet annihilation or thermal energy absorption to give fluorescent emission. In an organic electroluminescent device, above all, it is considered that a delayed fluorescent material capable of being thermally activated through thermal energy absorption would be especially useful. In the case where a delayed fluorescent material is used in an organic electroluminescent device, the exciton in an excited singlet state therein emits fluorescence in an ordinary manner. On the other hand, the exciton in an excited triplet state therein absorbs the heat generated by the device to cause intersystem crossing toward an excited singlet state, thereby emitting fluorescence. In this case, the light emission is from the excited singlet state and is therefore at the same wavelength as that of fluorescence, while, on the other hand, owing to the reverse intersystem crossing from the excited triplet state to the excited singlet state, the life of the resultant light (light emission life) is longer than that of ordinary fluorescence or phosphorescence, that is, the light is observed as a delayed fluorescent light. The phenomenon may be defined as delayed fluorescence. Using such a thermally-activating exciton transfer mechanism, the ratio of the compound in an excited singlet state, which is generally formed only in a ratio of 25%, may be increased up to 25% or more through thermal energy absorption after carrier injection. Using a compound capable of emitting strong fluorescence or delayed fluorescence even at a low temperature of lower than 100° C., intersystem crossing from the excited triplet state to an excited singlet state may occur sufficiently by heat of the device to emit delayed fluorescence, and in the case, the light emission efficiency can be markedly increased.

In addition, in the present invention, a hole blocking layer containing a compound represented by the general formula (1) is formed to be in adjacent to the light emitting layer on the cathode side, and accordingly, the exciton in an excited triplet state and the exciton in an excited singlet state forming in the light emitting layer can be prevented from diffusing toward the cathode side, and reverse intersystem crossing from the excited triplet state to the excited singlet state and radiation deactivation of the exciton in the excited singlet state occur at a high degree of probability. Consequently, the light emission efficiency can be more increased.

In the following, the light emitting material usable in the light emitting layer is described. A light emitting material is used in the light emitting layer. The light emitting material may be a delayed fluorescent material that emits delayed fluorescence or a fluorescent material that does not emit delayed fluorescence.

The kind of the delayed fluorescent material usable in the light emitting layer is not specifically limited. The compounds represented by the general formula (1) may be used as delayed fluorescent materials. Preferred examples of delayed fluorescent materials include compounds described in paragraphs 0008 to 0048 and 0095 to 0133 in WO2013/154064, paragraphs 0007 to 0047 and 0073 to 0085 in WO2013/011954, paragraphs 0007 to 0033 and 0059 to 0066 in WO2013/011955, paragraphs 0008 to 0071 and 0118 to 0133 in WO2013/081088, paragraphs 0009 to 0046 and 0093 to 0134 in JP 2013-256490 A, paragraphs 0008 to 0020 and 0038 to 0040 in JP 2013-116975 A, paragraphs 0007 to 0032 and 0079 to 0084 in WO2013/133359, paragraphs 0008 to 0054 and 0101 to 0121 in WO2013/161437, paragraphs 0007 to 0041 and 0060 to 0069 in JP 2014-9352 A, and paragraphs 0008 to 0048 and 0067 to 0076 in JP 2014-9224 A, especially exemplified compounds therein to emit delayed fluorescence. Also preferably used herein are light emitting materials capable of emitting delayed fluorescence as described in JP 2013-253121 A, WO2013/133359, WO2014/034535, WO2014/115743, WO2014/122895, WO2014/126200, WO2014/136758, WO2014/133121, WO2014/136860, WO2014/196585, WO2014/189122, WO2014/168101, WO2015/008580, WO2014/203840, WO2015/002213, WO2015/016200, WO2015/019725, WO2015/072470, WO2015/108049, WO2015/080182, WO2015/072537, WO2015/080183, JP 2015-129240 A, WO2015/129714, WO2015/129715, WO2015/133501, WO2015/136880, WO2015/137244, WO2015/137202, WO2015/137136, WO2015/146541, and WO2015/159541. The patent publications described in this section are incorporated herein as a part of this description by reference.

Further, compounds represented by the following general formulae (A) to (F) and compounds having a structure mentioned below may also be employed as light emitting materials. In particular, those emitting delayed fluorescence are preferably employed.

First, compounds represented by the following general formula (A) are described.

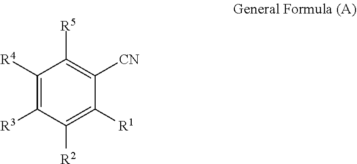

In the general formula (A), at least one of R¹ to R⁵ represents a cyano group, at least one of R¹ to R⁵ represents a group represented by the following general formula (11), and the remaining R¹ to R⁵ each represent a hydrogen atom or a substituent.

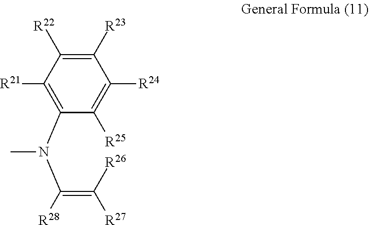

In the general formula (11), R²¹ to R²⁸ each independently represent a hydrogen atom or a substituent. However, these satisfy at least one of the following <A> or <B>.
<A> R²⁵ and R²⁶ together form a single bond.
<B> R²⁷ and R²⁸ together form an atomic group necessary for forming a substituted or unsubstituted benzene ring.

Examples of the group represented by the general formula (11) include groups represented by the following general formulae (12) to (15).

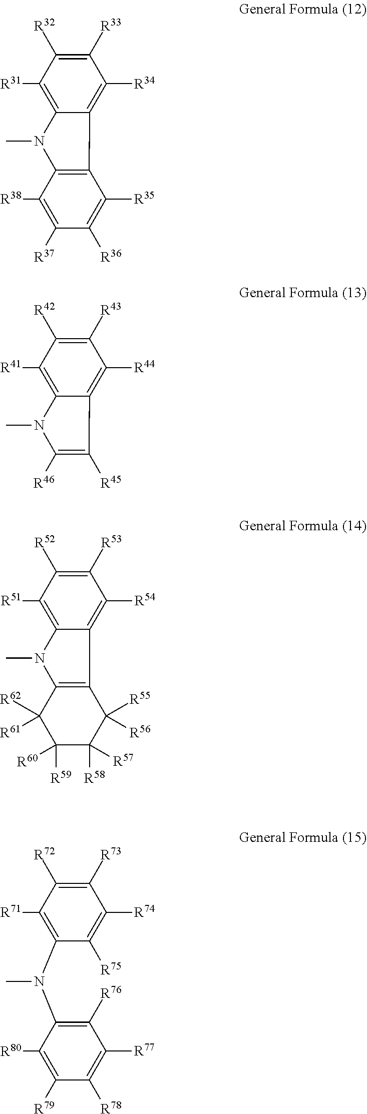

In the general formulae (12) to (15), R³¹ to R³⁸, R⁴¹ to R⁴⁶, R⁵¹ to R⁶² and R⁷¹ to R⁸⁰ each independently represent a hydrogen atom or a substituent. The substituting position and the number of the substituents, if any, in the group represented by the general formulae (12) to (15) are not specifically limited. In the case where the group has plural substituents, they may be the same as or different from each other.

Specific examples of the compounds represented by the general formula (A) includes compounds listed in the following Tables. In the Tables where the compound has two or more groups represented by any of the general formulae (12) to (15) in the molecule, these groups all have the same structure. For example, in the compound 1 of the general formula (1), R¹, R², R⁴ and R⁵ each are a group represented by the general formula (12) and these groups are all unsubstituted 9-carbazolyl groups. In the Tables, those of formulae (21) to (24) are as mentioned below. n indicates a recurring unit number and is an integer of 2 or more.

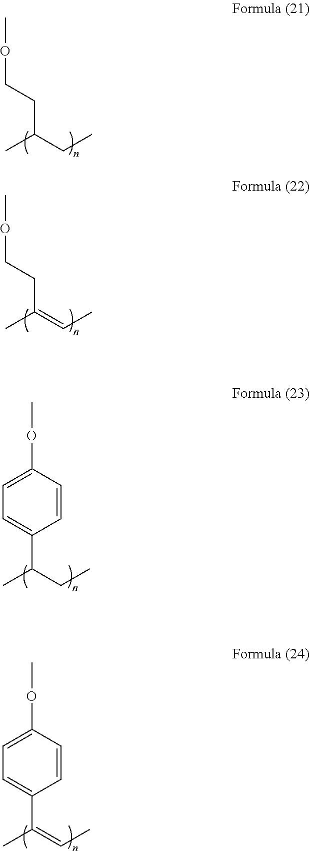

TABLE 2

| | | |
|---|---|---|
| Compound | General Formula (1) | General Formula (12) |

| No. | R¹ | R² | R³ | R⁴ | R⁵ | R³¹, R³⁸ | R³², R³⁷ | R³³, R³⁶ | R³⁴, R³⁵ |
|---|---|---|---|---|---|---|---|---|---|
| 1 | General Formula (12) | General Formula (12) | CN | General Formula (12) | General Formula (12) | H | H | H | H |
| 2 | General Formula (12) | General Formula (12) | CN | General Formula (12) | General Formula (12) | H | CH₃ | H | H |
| 3 | General Formula (12) | General Formula (12) | CN | General Formula (12) | General Formula (12) | H | CH₃O | H | H |
| 4 | General Formula (12) | General Formula (12) | CN | General Formula (12) | General Formula (12) | H | H | CH₃ | H |
| 5 | General Formula (12) | General Formula (12) | CN | General Formula (12) | General Formula (12) | H | H | CH₃O | H |
| 6 | General Formula (12) | General Formula (12) | CN | General Formula (12) | General Formula (12) | H | H | t-C₄H₉ | H |
| 7 | General Formula (12) | General Formula (12) | CN | General Formula (12) | General Formula (12) | H | H | Cl | H |
| 8 | General Formula (12) | General Formula (12) | CN | General Formula (12) | General Formula (12) | H | H | F | H |
| 9 | General Formula (12) | General Formula (12) | CN | General Formula (12) | General Formula (12) | H | H | H | CH₃ |
| 10 | General Formula (12) | General Formula (12) | CN | General Formula (12) | General Formula (12) | H | H | H | CH₃O |
| 11 | General Formula (12) | General Formula (12) | CN | General Formula (12) | H | H | H | H | H |
| 12 | General Formula (12) | General Formula (12) | CN | General Formula (12) | H | H | CH₃ | H | H |
| 13 | General Formula (12) | General Formula (12) | CN | General Formula (12) | H | H | CH₃O | H | H |
| 14 | General Formula (12) | General Formula (12) | CN | General Formula (12) | H | H | H | CH₃ | H |
| 15 | General Formula (12) | General Formula (12) | CN | General Formula (12) | H | H | H | CH₃O | H |
| 16 | General Formula (12) | General Formula (12) | CN | General Formula (12) | H | H | H | t-C₄H₉ | H |
| 17 | General Formula (12) | General Formula (12) | CN | General Formula (12) | H | H | H | Cl | H |
| 18 | General Formula (12) | General Formula (12) | CN | General Formula (12) | H | H | H | F | H |
| 19 | General Formula (12) | General Formula (12) | CN | General Formula (12) | H | H | H | H | CH₃ |
| 20 | General Formula (12) | General Formula (12) | CN | General Formula (12) | H | H | H | H | CH₃O |
| 21 | General Formula (12) | General Formula (12) | CN | H | H | H | H | H | H |
| 22 | General Formula (12) | General Formula (12) | CN | H | H | H | CH₃ | H | H |
| 23 | General Formula (12) | General Formula (12) | CN | H | H | H | CH₃O | H | H |
| 24 | General Formula (12) | General Formula (12) | CN | H | H | H | H | CH₃ | H |
| 25 | General Formula (12) | General Formula (12) | CN | H | H | H | H | CH₃O | H |
| 26 | General Formula (12) | General Formula (12) | CN | H | H | H | H | t-C₄H₉ | H |
| 27 | General Formula (12) | General Formula (12) | CN | H | H | H | H | Cl | H |
| 28 | General Formula (12) | General Formula (12) | CN | H | H | H | H | F | H |
| 29 | General Formula (12) | General Formula (12) | CN | H | H | H | H | H | CH₃ |
| 30 | General Formula (12) | General Formula (12) | CN | H | H | H | H | H | CH₃O |
| 31 | General Formula (12) | H | CN | General Formula (12) | H | H | H | H | H |
| 32 | General Formula (12) | H | CN | General Formula (12) | H | H | CH₃ | H | H |
| 33 | General Formula (12) | H | CN | General Formula (12) | H | H | CH₃O | H | H |
| 34 | General Formula (12) | H | CN | General Formula (12) | H | H | H | CH₃ | H |
| 35 | General Formula (12) | H | CN | General Formula (12) | H | H | H | CH₃O | H |
| 36 | General Formula (12) | H | CN | General Formula (12) | H | H | H | t-C₄H₉ | H |
| 37 | General Formula (12) | H | CN | General Formula (12) | H | H | H | Cl | H |
| 38 | General Formula (12) | H | CN | General Formula (12) | H | H | H | F | H |
| 39 | General Formula (12) | H | CN | General Formula (12) | H | H | H | H | CH₃ |
| 40 | General Formula (12) | H | CN | General Formula (12) | H | H | H | H | CH₃O |
| 41 | General Formula (12) | H | CN | H | General Formula (12) | H | H | H | H |
| 42 | General Formula (12) | H | CN | H | General Formula (12) | H | CH₃ | H | H |
| 43 | General Formula (12) | H | CN | H | General Formula (12) | H | CH₃O | H | H |
| 44 | General Formula (12) | H | CN | H | General Formula (12) | H | H | CH₃ | H |
| 45 | General Formula (12) | H | CN | H | General Formula (12) | H | H | CH₃O | H |
| 46 | General Formula (12) | H | CN | H | General Formula (12) | H | H | t-C₄H₉ | H |
| 47 | General Formula (12) | H | CN | H | General Formula (12) | H | H | Cl | H |
| 48 | General Formula (12) | H | CN | H | General Formula (12) | H | H | F | H |
| 49 | General Formula (12) | H | CN | H | General Formula (12) | H | H | H | CH₃ |
| 50 | General Formula (12) | H | CN | H | General Formula (12) | H | H | H | CH₃O |
| 51 | General Formula (12) | H | CN | H | H | H | H | H | H |
| 52 | General Formula (12) | H | CN | H | H | H | CH₃ | H | H |
| 53 | General Formula (12) | H | CN | H | H | H | CH₃O | H | H |
| 54 | General Formula (12) | H | CN | H | H | H | H | CH₃ | H |
| 55 | General Formula (12) | H | CN | H | H | H | H | CH₃O | H |
| 56 | General Formula (12) | H | CN | H | H | H | H | t-C₄H₉ | H |
| 57 | General Formula (12) | H | CN | H | H | H | H | Cl | H |
| 58 | General Formula (12) | H | CN | H | H | H | H | F | H |
| 59 | General Formula (12) | H | CN | H | H | H | H | H | CH₃ |
| 60 | General Formula (12) | H | CN | H | H | H | H | H | CH₃O |
| 61 | General Formula (12) | General Formula (12) | CN | General Formula (12) | F | H | H | H | H |
| 62 | General Formula (12) | General Formula (12) | CN | General Formula (12) | F | H | CH₃ | H | H |
| 63 | General Formula (12) | General Formula (12) | CN | General Formula (12) | F | H | CH₃O | H | H |
| 64 | General Formula (12) | General Formula (12) | CN | General Formula (12) | F | H | H | CH₃ | H |
| 65 | General Formula (12) | General Formula (12) | CN | General Formula (12) | F | H | H | CH₃O | H |
| 66 | General Formula (12) | General Formula (12) | CN | General Formula (12) | F | H | H | t-C₄H₉ | H |
| 67 | General Formula (12) | General Formula (12) | CN | General Formula (12) | F | H | H | Cl | H |
| 68 | General Formula (12) | General Formula (12) | CN | General Formula (12) | F | H | H | F | H |
| 69 | General Formula (12) | General Formula (12) | CN | General Formula (12) | F | H | H | H | CH₃ |
| 70 | General Formula (12) | General Formula (12) | CN | General Formula (12) | F | H | H | H | CH₃O |
| 71 | General Formula (12) | General Formula (12) | CN | F | F | H | H | H | H |
| 72 | General Formula (12) | General Formula (12) | CN | F | F | H | CH₃ | H | H |
| 73 | General Formula (12) | General Formula (12) | CN | F | F | H | CH₃O | H | H |
| 74 | General Formula (12) | General Formula (12) | CN | F | F | H | H | CH₃ | H |
| 75 | General Formula (12) | General Formula (12) | CN | F | F | H | H | CH₃O | H |
| 76 | General Formula (12) | General Formula (12) | CN | F | F | H | H | t-C₄H₉ | H |
| 77 | General Formula (12) | General Formula (12) | CN | F | F | H | H | Cl | H |
| 78 | General Formula (12) | General Formula (12) | CN | F | F | H | H | F | H |
| 79 | General Formula (12) | General Formula (12) | CN | F | F | H | H | H | CH₃ |
| 80 | General Formula (12) | General Formula (12) | CN | F | F | H | H | H | CH₃O |
| 81 | General Formula (12) | F | CN | General Formula (12) | F | H | H | H | H |
| 82 | General Formula (12) | F | CN | General Formula (12) | F | H | CH₃ | H | H |
| 83 | General Formula (12) | F | CN | General Formula (12) | F | H | CH₃O | H | H |
| 84 | General Formula (12) | F | CN | General Formula (12) | F | H | H | CH₃ | H |
| 85 | General Formula (12) | F | CN | General Formula (12) | F | H | H | CH₃O | H |
| 86 | General Formula (12) | F | CN | General Formula (12) | F | H | H | t-C₄H₉ | H |
| 87 | General Formula (12) | F | CN | General Formula (12) | F | H | H | Cl | H |
| 88 | General Formula (12) | F | CN | General Formula (12) | F | H | H | F | H |
| 89 | General Formula (12) | F | CN | General Formula (12) | F | H | H | H | CH₃ |
| 90 | General Formula (12) | F | CN | General Formula (12) | F | H | H | H | CH₃O |
| 91 | General Formula (12) | F | CN | F | General Formula (12) | H | H | H | H |
| 92 | General Formula (12) | F | CN | F | General Formula (12) | H | CH₃ | H | H |
| 93 | General Formula (12) | F | CN | F | General Formula (12) | H | CH₃O | H | H |
| 94 | General Formula (12) | F | CN | F | General Formula (12) | H | H | CH₃ | H |
| 95 | General Formula (12) | F | CN | F | General Formula (12) | H | H | CH₃O | H |
| 96 | General Formula (12) | F | CN | F | General Formula (12) | H | H | t-C₄H₉ | H |
| 97 | General Formula (12) | F | CN | F | General Formula (12) | H | H | Cl | H |
| 98 | General Formula (12) | F | CN | F | General Formula (12) | H | H | F | H |
| 99 | General Formula (12) | F | CN | F | General Formula (12) | H | H | H | CH₃ |
| 100 | General Formula (12) | F | CN | F | General Formula (12) | H | H | H | CH₃O |
| 101 | General Formula (12) | F | CN | F | F | H | H | H | H |
| 102 | General Formula (12) | F | CN | F | F | H | CH₃ | H | H |
| 103 | General Formula (12) | F | CN | F | F | H | CH₃O | H | H |
| 104 | General Formula (12) | F | CN | F | F | H | H | CH₃ | H |
| 105 | General Formula (12) | F | CN | F | F | H | H | CH₃O | H |
| 106 | General Formula (12) | F | CN | F | F | H | H | t-C₄H₉ | H |
| 107 | General Formula (12) | F | CN | F | F | H | H | Cl | H |
| 108 | General Formula (12) | F | CN | F | F | H | H | F | H |
| 109 | General Formula (12) | F | CN | F | F | H | H | H | CH₃ |
| 110 | General Formula (12) | F | CN | F | F | H | H | H | CH₃O |
| 111 | General Formula (12) | General Formula (12) | CN | General Formula (12) | OH | H | H | H | H |
| 112 | General Formula (12) | General Formula (12) | CN | General Formula (12) | OH | H | CH₃ | H | H |
| 113 | General Formula (12) | General Formula (12) | CN | General Formula (12) | OH | H | CH₃O | H | H |
| 114 | General Formula (12) | General Formula (12) | CN | General Formula (12) | OH | H | H | CH₃ | H |
| 115 | General Formula (12) | General Formula (12) | CN | General Formula (12) | OH | H | H | CH₃O | H |
| 116 | General Formula (12) | General Formula (12) | CN | General Formula (12) | OH | H | H | t-C₄H₉ | H |
| 117 | General Formula (12) | General Formula (12) | CN | General Formula (12) | OH | H | H | Cl | H |
| 118 | General Formula (12) | General Formula (12) | CN | General Formula (12) | OH | H | H | F | H |
| 119 | General Formula (12) | General Formula (12) | CN | General Formula (12) | OH | H | H | H | CH₃ |
| 120 | General Formula (12) | General Formula (12) | CN | General Formula (12) | OH | H | H | H | CH₃O |
| 121 | General Formula (12) | General Formula (12) | CN | OH | OH | H | H | H | H |
| 122 | General Formula (12) | General Formula (12) | CN | OH | OH | H | CH₃ | H | H |
| 123 | General Formula (12) | General Formula (12) | CN | OH | OH | H | CH₃O | H | H |
| 124 | General Formula (12) | General Formula (12) | CN | OH | OH | H | H | CH₃ | H |
| 125 | General Formula (12) | General Formula (12) | CN | OH | OH | H | H | CH₃O | H |
| 126 | General Formula (12) | General Formula (12) | CN | OH | OH | H | H | t-C₄H₉ | H |
| 127 | General Formula (12) | General Formula (12) | CN | OH | OH | H | H | Cl | H |
| 128 | General Formula (12) | General Formula (12) | CN | OH | OH | H | H | F | H |
| 129 | General Formula (12) | General Formula (12) | CN | OH | OH | H | H | H | CH₃ |
| 130 | General Formula (12) | General Formula (12) | CN | OH | OH | H | H | H | CH₃O |
| 131 | General Formula (12) | OH | CN | General Formula (12) | OH | H | H | H | H |
| 132 | General Formula (12) | OH | CN | General Formula (12) | OH | H | CH₃ | H | H |
| 133 | General Formula (12) | OH | CN | General Formula (12) | OH | H | CH₃O | H | H |
| 134 | General Formula (12) | OH | CN | General Formula (12) | OH | H | H | CH₃ | H |
| 135 | General Formula (12) | OH | CN | General Formula (12) | OH | H | H | CH₃O | H |
| 136 | General Formula (12) | OH | CN | General Formula (12) | OH | H | H | t-C₄H₉ | H |
| 137 | General Formula (12) | OH | CN | General Formula (12) | OH | H | H | Cl | H |
| 138 | General Formula (12) | OH | CN | General Formula (12) | OH | H | H | F | H |
| 139 | General Formula (12) | OH | CN | General Formula (12) | OH | H | H | H | CH₃ |
| 140 | General Formula (12) | OH | CN | General Formula (12) | OH | H | H | H | CH₃O |
| 141 | General Formula (12) | OH | CN | OH | General Formula (12) | H | H | H | H |
| 142 | General Formula (12) | OH | CN | OH | General Formula (12) | H | CH₃ | H | H |
| 143 | General Formula (12) | OH | CN | OH | General Formula (12) | H | CH₃O | H | H |
| 144 | General Formula (12) | OH | CN | OH | General Formula (12) | H | H | CH₃ | H |
| 145 | General Formula (12) | OH | CN | OH | General Formula (12) | H | H | CH₃O | H |
| 146 | General Formula (12) | OH | CN | OH | General Formula (12) | H | H | t-C₄H₉ | H |
| 147 | General Formula (12) | OH | CN | OH | General Formula (12) | H | H | Cl | H |
| 148 | General Formula (12) | OH | CN | OH | General Formula (12) | H | H | F | H |
| 149 | General Formula (12) | OH | CN | OH | General Formula (12) | H | H | H | CH₃ |
| 150 | General Formula (12) | OH | CN | OH | General Formula (12) | H | H | H | CH3O |
| 151 | General Formula (12) | OH | CN | OH | OH | H | H | H | H |
| 152 | General Formula (12) | OH | CN | OH | OH | H | CH₃ | H | H |
| 153 | General Formula (12) | OH | CN | OH | OH | H | CH₃O | H | H |
| 154 | General Formula (12) | OH | CN | OH | OH | H | H | CH₃ | H |
| 155 | General Formula (12) | OH | CN | OH | OH | H | H | CH₃O | H |
| 156 | General Formula (12) | OH | CN | OH | OH | H | H | t-C₄H₉ | H |
| 157 | General Formula (12) | OH | CN | OH | OH | H | H | Cl | H |
| 158 | General Formula (12) | OH | CN | OH | OH | H | H | F | H |
| 159 | General Formula (12) | OH | CN | OH | OH | H | H | H | CH₃ |
| 160 | General Formula (12) | OH | CN | OH | OH | H | H | H | CH₃O |
| 161 | General Formula (12) | General Formula (12) | CN | General Formula (12) | Cl | H | H | H | H |
| 162 | General Formula (12) | General Formula (12) | CN | General Formula (12) | Cl | H | CH₃ | H | H |
| 163 | General Formula (12) | General Formula (12) | CN | General Formula (12) | Cl | H | CH₃O | H | H |
| 164 | General Formula (12) | General Formula (12) | CN | General Formula (12) | Cl | H | H | CH₃ | H |
| 165 | General Formula (12) | General Formula (12) | CN | General Formula (12) | Cl | H | H | CH₃O | H |
| 166 | General Formula (12) | General Formula (12) | CN | General Formula (12) | Cl | H | H | t-C₄H₉ | H |
| 167 | General Formula (12) | General Formula (12) | CN | General Formula (12) | Cl | H | H | Cl | H |
| 168 | General Formula (12) | General Formula (12) | CN | General Formula (12) | Cl | H | H | F | H |
| 169 | General Formula (12) | General Formula (12) | CN | General Formula (12) | Cl | H | H | H | CH₃ |
| 170 | General Formula (12) | General Formula (12) | CN | General Formula (12) | Cl | H | H | H | CH₃O |
| 171 | General Formula (12) | General Formula (12) | CN | General Formula (12) | F | H | H | H | H |
| 172 | General Formula (12) | General Formula (12) | CN | General Formula (12) | F | H | CH₃ | H | H |
| 173 | General Formula (12) | General Formula (12) | CN | General Formula (12) | F | H | CH₃O | H | H |
| 174 | General Formula (12) | General Formula (12) | CN | General Formula (12) | F | H | H | CH₃ | H |
| 175 | General Formula (12) | General Formula (12) | CN | General Formula (12) | F | H | H | CH₃O | H |
| 176 | General Formula (12) | General Formula (12) | CN | General Formula (12) | F | H | H | t-C₄H₉ | H |
| 177 | General Formula (12) | General Formula (12) | CN | General Formula (12) | F | H | H | Cl | H |
| 178 | General Formula (12) | General Formula (12) | CN | General Formula (12) | F | H | H | F | H |
| 179 | General Formula (12) | General Formula (12) | CN | General Formula (12) | F | H | H | H | CH₃ |
| 180 | General Formula (12) | General Formula (12) | CN | General Formula (12) | F | H | H | H | CH₃O |
| 181 | General Formula (12) | General Formula (12) | CN | General Formula (12) | CH₃O | H | H | H | H |
| 182 | General Formula (12) | General Formula (12) | CN | General Formula (12) | CH₃O | H | CH₃ | H | H |
| 183 | General Formula (12) | General Formula (12) | CN | General Formula (12) | CH₃O | H | CH₃O | H | H |
| 184 | General Formula (12) | General Formula (12) | CN | General Formula (12) | CH₃O | H | H | CH₃ | H |
| 185 | General Formula (12) | General Formula (12) | CN | General Formula (12) | CH₃O | H | H | CH₃O | H |
| 186 | General Formula (12) | General Formula (12) | CN | General Formula (12) | CH₃O | H | H | t-C₄H₉ | H |
| 187 | General Formula (12) | General Formula (12) | CN | General Formula (12) | CH₃O | H | H | Cl | H |
| 188 | General Formula (12) | General Formula (12) | CN | General Formula (12) | CH₃O | H | H | F | H |
| 189 | General Formula (12) | General Formula (12) | CN | General Formula (12) | C₂H₅O | H | H | H | CH₃ |
| 190 | General Formula (12) | General Formula (12) | CN | General Formula (12) | C₂H₅O | H | H | H | CH₃O |
| 191 | General Formula (12) | General Formula (12) | CN | General Formula (12) | C₂H₅O | H | H | H | H |
| 192 | General Formula (12) | General Formula (12) | CN | General Formula (12) | C₂H₅O | H | CH₃ | H | H |
| 193 | General Formula (12) | General Formula (12) | CN | General Formula (12) | C₂H₅O | H | CH₃O | H | H |
| 194 | General Formula (12) | General Formula (12) | CN | General Formula (12) | C₂H₅O | H | H | CH₃ | H |
| 195 | General Formula (12) | General Formula (12) | CN | General Formula (12) | C₂H₅O | H | H | CH₃O | H |
| 196 | General Formula (12) | General Formula (12) | CN | General Formula (12) | C₂H₅O | H | H | t-C₄H₉ | H |
| 197 | General Formula (12) | General Formula (12) | CN | General Formula (12) | C₂H₅O | H | H | Cl | H |
| 198 | General Formula (12) | General Formula (12) | CN | General Formula (12) | C₂H₅O | H | H | F | H |
| 199 | General Formula (12) | General Formula (12) | CN | General Formula (12) | C₂H₅O | H | H | H | CH₃ |
| 200 | General Formula (12) | General Formula (12) | CN | General Formula (12) | C₂H₅O | H | H | H | CH₃O |
| 201 | General Formula (12) | General Formula (12) | CN | General Formula (12) | C₆H₅O | H | H | H | H |
| 202 | General Formula (12) | General Formula (12) | CN | General Formula (12) | C₆H₅O | H | CH₃ | H | H |
| 203 | General Formula (12) | General Formula (12) | CN | General Formula (12) | C₆H₅O | H | CH₃O | H | H |
| 204 | General Formula (12) | General Formula (12) | CN | General Formula (12) | C₆H₅O | H | H | CH₃ | H |
| 205 | General Formula (12) | General Formula (12) | CN | General Formula (12) | C₆H₅O | H | H | CH₃O | H |
| 206 | General Formula (12) | General Formula (12) | CN | General Formula (12) | C₆H₅O | H | H | t-C₄H₉ | H |
| 207 | General Formula (12) | General Formula (12) | CN | General Formula (12) | C₆H₅O | H | H | Cl | H |
| 208 | General Formula (12) | General Formula (12) | CN | General Formula (12) | C₆H₅O | H | H | F | H |
| 209 | General Formula (12) | General Formula (12) | CN | General Formula (12) | C₆H₅O | H | H | H | CH₃ |
| 210 | General Formula (12) | General Formula (12) | CN | General Formula (12) | C₆H₅O | H | H | H | CH₃O |
| 211 | General Formula (12) | General Formula (12) | CN | General Formula (12) | Formula (21) | H | H | H | H |
| 212 | General Formula (12) | General Formula (12) | CN | General Formula (12) | Formula (21) | H | CH₃ | H | H |
| 213 | General Formula (12) | General Formula (12) | CN | General Formula (12) | Formula (21) | H | CH₃O | H | H |
| 214 | General Formula (12) | General Formula (12) | CN | General Formula (12) | Formula (21) | H | H | CH₃ | H |
| 215 | General Formula (12) | General Formula (12) | CN | General Formula (12) | Formula (21) | H | H | CH₃O | H |
| 216 | General Formula (12) | General Formula (12) | CN | General Formula (12) | Formula (21) | H | H | t-C₄H₉ | H |
| 217 | General Formula (12) | General Formula (12) | CN | General Formula (12) | Formula (21) | H | H | Cl | H |
| 218 | General Formula (12) | General Formula (12) | CN | General Formula (12) | Formula (21) | H | H | F | H |
| 219 | General Formula (12) | General Formula (12) | CN | General Formula (12) | Formula (21) | H | H | H | CH₃ |
| 220 | General Formula (12) | General Formula (12) | CN | General Formula (12) | Formula (21) | H | H | H | CH₃O |
| 221 | General Formula (12) | General Formula (12) | CN | General Formula (12) | Formula (22) | H | H | H | H |
| 222 | General Formula (12) | General Formula (12) | CN | General Formula (12) | Formula (22) | H | CH₃ | H | H |
| 223 | General Formula (12) | General Formula (12) | CN | General Formula (12) | Formula (22) | H | CH₃O | H | H |
| 224 | General Formula (12) | General Formula (12) | CN | General Formula (12) | Formula (22) | H | H | CH₃ | H |
| 225 | General Formula (12) | General Formula (12) | CN | General Formula (12) | Formula (22) | H | H | CH₃O | H |
| 226 | General Formula (12) | General Formula (12) | CN | General Formula (12) | Formula (22) | H | H | t-C₄H₉ | H |
| 227 | General Formula (12) | General Formula (12) | CN | General Formula (12) | Formula (22) | H | H | Cl | H |
| 228 | General Formula (12) | General Formula (12) | CN | General Formula (12) | Formula (22) | H | H | F | H |
| 229 | General Formula (12) | General Formula (12) | CN | General Formula (12) | Formula (22) | H | H | H | CH₃ |
| 230 | General Formula (12) | General Formula (12) | CN | General Formula (12) | Formula (22) | H | H | H | CH₃O |
| 231 | General Formula (12) | General Formula (12) | CN | General Formula (12) | Formula (23) | H | H | H | H |
| 232 | General Formula (12) | General Formula (12) | CN | General Formula (12) | Formula (23) | H | CH₃ | H | H |
| 233 | General Formula (12) | General Formula (12) | CN | General Formula (12) | Formula (23) | H | CH₃O | H | H |
| 234 | General Formula (12) | General Formula (12) | CN | General Formula (12) | Formula (23) | H | H | CH₃ | H |
| 235 | General Formula (12) | General Formula (12) | CN | General Formula (12) | Formula (23) | H | H | CH₃O | H |
| 236 | General Formula (12) | General Formula (12) | CN | General Formula (12) | Formula (23) | H | H | t-C₄H₉ | H |
| 237 | General Formula (12) | General Formula (12) | CN | General Formula (12) | Formula (23) | H | H | Cl | H |
| 238 | General Formula (12) | General Formula (12) | CN | General Formula (12) | Formula (23) | H | H | F | H |
| 239 | General Formula (12) | General Formula (12) | CN | General Formula (12) | Formula (23) | H | H | H | CH₃ |
| 240 | General Formula (12) | General Formula (12) | CN | General Formula (12) | Formula (23) | H | H | H | CH₃O |
| 241 | General Formula (12) | General Formula (12) | CN | General Formula (12) | Formula (24) | H | H | H | H |
| 242 | General Formula (12) | General Formula (12) | CN | General Formula (12) | Formula (24) | H | CH₃ | H | H |
| 243 | General Formula (12) | General Formula (12) | CN | General Formula (12) | Formula (24) | H | CH₃O | H | H |
| 244 | General Formula (12) | General Formula (12) | CN | General Formula (12) | Formula (24) | H | H | CH₃ | H |
| 245 | General Formula (12) | General Formula (12) | CN | General Formula (12) | Formula (24) | H | H | CH₃O | H |
| 246 | General Formula (12) | General Formula (12) | CN | General Formula (12) | Formula (24) | H | H | t-C₄H₉ | H |
| 247 | General Formula (12) | General Formula (12) | CN | General Formula (12) | Formula (24) | H | H | Cl | H |
| 248 | General Formula (12) | General Formula (12) | CN | General Formula (12) | Formula (24) | H | H | F | H |
| 249 | General Formula (12) | General Formula (12) | CN | General Formula (12) | Formula (24) | H | H | H | CH₃ |
| 250 | General Formula (12) | General Formula (12) | CN | General Formula (12) | Formula (24) | H | H | H | CH₃O |
| 251 | General Formula (12) | General Formula (12) | CN | General Formula (12) | General Formula (12) | H | C₆H₅ | H | H |
| 252 | General Formula (12) | General Formula (12) | CN | General Formula (12) | General Formula (12) | H | H | C₆H₅ | H |
| 253 | General Formula (12) | General Formula (12) | CN | General Formula (12) | H | H | C₆H₅ | H | H |
| 254 | General Formula (12) | General Formula (12) | CN | General Formula (12) | H | H | H | C₆H₅ | H |
| 255 | General Formula (12) | General Formula (12) | CN | H | H | H | C₆H₅ | H | H |
| 256 | General Formula (12) | General Formula (12) | CN | H | H | H | H | C₆H₅ | H |
| 257 | General Formula (12) | H | CN | General Formula (12) | H | H | C₆H₅ | H | H |
| 258 | General Formula (12) | H | CN | General Formula (12) | H | H | H | C₆H₅ | H |
| 259 | General Formula (12) | H | CN | H | General Formula (12) | H | C₆H₅ | H | H |
| 260 | General Formula (12) | H | CN | H | General Formula (12) | H | H | C₆H₅ | H |
| 261 | General Formula (12) | H | CN | H | H | H | C₆H₅ | H | H |
| 262 | General Formula (12) | H | CN | H | H | H | H | C₆H₅ | H |
| 263 | General Formula (12) | General Formula (12) | CN | General Formula (12) | F | H | C₆H₅ | H | H |
| 264 | General Formula (12) | General Formula (12) | CN | General Formula (12) | F | H | H | C₆H₅ | H |
| 265 | General Formula (12) | General Formula (12) | CN | F | F | H | C₆H₅ | H | H |
| 266 | General Formula (12) | General Formula (12) | CN | F | F | H | H | C₆H₅ | H |
| 267 | General Formula (12) | F | CN | General Formula (12) | F | H | C₆H₅ | H | H |
| 268 | General Formula (12) | F | CN | General Formula (12) | F | H | H | C₆H₅ | H |
| 269 | General Formula (12) | F | CN | F | General Formula (12) | H | C₆H₅ | H | H |
| 270 | General Formula (12) | F | CN | F | General Formula (12) | H | H | C₆H₅ | H |
| 271 | General Formula (12) | F | CN | F | F | H | C₆H₅ | H | H |
| 272 | General Formula (12) | F | CN | F | F | H | H | C₆H₅ | H |
| 273 | General Formula (12) | General Formula (12) | CN | General Formula (12) | OH | H | C₆H₅ | H | H |
| 274 | General Formula (12) | General Formula (12) | CN | General Formula (12) | OH | H | H | C₆H₅ | H |
| 275 | General Formula (12) | General Formula (12) | CN | OH | OH | H | C₆H₅ | H | H |
| 276 | General Formula (12) | General Formula (12) | CN | OH | OH | H | H | C₆H₅ | H |
| 277 | General Formula (12) | OH | CN | General Formula (12) | OH | H | C₆H₅ | H | H |
| 278 | General Formula (12) | OH | CN | General Formula (12) | OH | H | H | C₆H₅ | H |
| 279 | General Formula (12) | OH | CN | OH | General Formula (12) | H | C₆H₅ | H | H |
| 280 | General Formula (12) | OH | CN | OH | General Formula (12) | H | H | C₆H₅ | H |
| 281 | General Formula (12) | OH | CN | OH | OH | H | C₆H₅ | H | H |
| 282 | General Formula (12) | OH | CN | OH | OH | H | H | C₆H₅ | H |
| 283 | General Formula (12) | General Formula (12) | CN | General Formula (12) | Cl | H | C₆H₅ | H | H |
| 284 | General Formula (12) | General Formula (12) | CN | General Formula (12) | Cl | H | H | C₆H₅ | H |
| 285 | General Formula (12) | General Formula (12) | CN | General Formula (12) | F | H | C₆H₅ | H | H |
| 286 | General Formula (12) | General Formula (12) | CN | General Formula (12) | F | H | H | C₆H₅ | H |
| 287 | General Formula (12) | General Formula (12) | CN | General Formula (12) | CH₃O | H | C₆H₅ | H | H |
| 288 | General Formula (12) | General Formula (12) | CN | General Formula (12) | CH₃O | H | H | C₆H₅ | H |
| 289 | General Formula (12) | General Formula (12) | CN | General Formula (12) | C₂H₅O | H | C₆H₅ | H | H |
| 290 | General Formula (12) | General Formula (12) | CN | General Formula (12) | C₂H₅O | H | H | C₆H₅ | H |
| 291 | General Formula (12) | General Formula (12) | CN | General Formula (12) | C₆H₅O | H | C₆H₅ | H | H |
| 292 | General Formula (12) | General Formula (12) | CN | General Formula (12) | C₆H₅O | H | H | C₆H₅ | H |
| 293 | General Formula (12) | General Formula (12) | CN | General Formula (12) | Formula (21) | H | C₆H₅ | H | H |
| 294 | General Formula (12) | General Formula (12) | CN | General Formula (12) | Formula (21) | H | H | C₆H₅ | H |
| 295 | General Formula (12) | General Formula (12) | CN | General Formula (12) | Formula (22) | H | C₆H₅ | H | H |
| 296 | General Formula (12) | General Formula (12) | CN | General Formula (12) | Formula (22) | H | H | C₆H₅ | H |
| 297 | General Formula (12) | General Formula (12) | CN | General Formula (12) | Formula (23) | H | C₆H₅ | H | H |
| 298 | General Formula (12) | General Formula (12) | CN | General Formula (12) | Formula (23) | H | H | C₆H₅ | H |
| 299 | General Formula (12) | General Formula (12) | CN | General Formula (12) | Formula (24) | H | C₆H₅ | H | H |
| 300 | General Formula (12) | General Formula (12) | CN | General Formula (12) | Formula (24) | H | H | C₆H₅ | H |

TABLE 3

| | | |
|---|---|---|
| Compound | General Formula (1) | General Formula (12) |

| No. | R¹ | R² | R³ | R⁴ | R⁵ | R³¹, R³⁸ | R³², R³⁷ | R³³, R³⁶ | R³⁴, R³⁵ |
|---|---|---|---|---|---|---|---|---|---|
| 301 | General Formula (12) | CN | General Formula (12) | General Formula (12) | General Formula (12) | H | H | H | H |
| 302 | General Formula (12) | CN | General Formula (12) | General Formula (12) | General Formula (12) | H | CH₃ | H | H |
| 303 | General Formula (12) | CN | General Formula (12) | General Formula (12) | General Formula (12) | H | CH₃O | H | H |
| 304 | General Formula (12) | CN | General Formula (12) | General Formula (12) | General Formula (12) | H | H | CH₃ | H |
| 305 | General Formula (12) | CN | General Formula (12) | General Formula (12) | General Formula (12) | H | H | CH₃O | H |
| 306 | General Formula (12) | CN | General Formula (12) | General Formula (12) | General Formula (12) | H | H | t-C₄H₉ | H |
| 307 | General Formula (12) | CN | General Formula (12) | General Formula (12) | General Formula (12) | H | H | Cl | H |
| 308 | General Formula (12) | CN | General Formula (12) | General Formula (12) | General Formula (12) | H | H | F | H |
| 309 | General Formula (12) | CN | General Formula (12) | General Formula (12) | General Formula (12) | H | H | H | CH₃ |
| 310 | General Formula (12) | CN | General Formula (12) | General Formula (12) | General Formula (12) | H | H | H | CH₃O |
| 311 | General Formula (12) | CN | General Formula (12) | General Formula (12) | H | H | H | H | H |
| 312 | General Formula (1) | CN | General Formula (12) | General Formula (12) | H | H | H | CH₃ | H |
| 313 | General Formula (1) | CN | General Formula (12) | General Formula (12) | H | H | H | CH₃O | H |
| 314 | General Formula (12) | CN | General Formula (12) | H | General Formula (12) | H | H | H | H |
| 315 | General Formula (12) | CN | General Formula (12) | H | General Formula (12) | H | H | CH₃ | H |
| 316 | General Formula (12) | CN | General Formula (12) | H | General Formula (12) | H | H | CH₃O | H |
| 317 | General Formula (12) | CN | H | General Formula (12) | General Formula (12) | H | H | H | H |
| 318 | General Formula (12) | CN | H | General Formula (12) | General Formula (12) | H | H | CH₃ | H |
| 319 | General Formula (12) | CN | H | General Formula (12) | General Formula (12) | H | H | CH₃O | H |
| 320 | H | CN | General Formula (12) | General Formula (12) | General Formula (12) | H | H | H | H |
| 321 | H | CN | General Formula (12) | General Formula (12) | General Formula (12) | H | H | CH₃ | H |
| 322 | H | CN | General Formula (12) | General Formula (12) | General Formula (12) | H | H | CH₃O | H |
| 323 | General Formula (12) | CN | General Formula (12) | H | H | H | H | H | H |
| 324 | General Formula (12) | CN | General Formula (12) | H | H | H | H | CH₃ | H |
| 325 | General Formula (12) | CN | General Formula (12) | H | H | H | H | CH₃O | H |
| 326 | General Formula (12) | CN | H | General Formula (12) | H | H | H | H | H |
| 327 | General Formula (12) | CN | H | General Formula (12) | H | H | H | CH₃ | H |
| 328 | General Formula (12) | CN | H | General Formula (12) | H | H | H | CH₃O | H |
| 329 | H | CN | General Formula (12) | General Formula (12) | H | H | H | H | H |
| 330 | H | CN | General Formula (12) | General Formula (12) | H | H | H | CH₃ | H |
| 331 | H | CN | General Formula (12) | General Formula (12) | H | H | H | CH₃O | H |
| 332 | General Formula (12) | CN | H | H | General Formula (12) | H | H | H | H |
| 333 | General Formula (12) | CN | H | H | General Formula (12) | H | H | CH₃ | H |
| 334 | General Formula (12) | CN | H | H | General Formula (12) | H | H | CH₃O | H |
| 335 | H | CN | General Formula (12) | H | General Formula (12) | H | H | H | H |
| 336 | H | CN | General Formula (12) | H | General Formula (12) | H | H | CH₃ | H |
| 337 | H | CN | General Formula (12) | H | General Formula (12) | H | H | CH₃O | H |
| 338 | H | CN | H | General Formula (12) | General Formula (12) | H | H | H | H |
| 339 | H | CN | H | General Formula (12) | General Formula (12) | H | H | CH₃ | H |
| 340 | H | CN | H | General Formula (12) | General Formula (12) | H | H | CH₃O | H |
| 341 | General Formula (12) | CN | H | H | H | H | H | H | H |
| 342 | General Formula (12) | CN | H | H | H | H | H | CH₃ | H |
| 343 | General Formula (12) | CN | H | H | H | H | H | CH₃O | H |
| 344 | H | CN | General Formula (12) | H | H | H | H | H | H |
| 345 | H | CN | General Formula (12) | H | H | H | H | CH₃ | H |
| 346 | H | CN | General Formula (12) | H | H | H | H | CH₃O | H |
| 347 | H | CN | H | General Formula (12) | H | H | H | H | H |
| 348 | H | CN | H | General Formula (12) | H | H | H | CH₃ | H |
| 349 | H | CN | H | General Formula (12) | H | H | H | CH₃O | H |
| 350 | General Formula (12) | CN | General Formula (12) | General Formula (12) | F | H | H | H | H |
| 351 | General Formula (12) | CN | General Formula (12) | General Formula (12) | F | H | H | CH₃ | H |
| 352 | General Formula (12) | CN | General Formula (12) | General Formula (12) | F | H | H | CH₃O | H |
| 353 | General Formula (12) | CN | General Formula (12) | F | General Formula (12) | H | H | H | H |
| 354 | General Formula (12) | CN | General Formula (12) | F | General Formula (12) | H | H | CH₃ | H |
| 355 | General Formula (12) | CN | General Formula (12) | F | General Formula (12) | H | H | CH₃O | H |
| 356 | General Formula (12) | CN | F | General Formula (12) | General Formula (12) | H | H | H | H |
| 357 | General Formula (12) | CN | F | General Formula (12) | General Formula (12) | H | H | CH₃ | H |
| 358 | General Formula (12) | CN | F | General Formula (12) | General Formula (12) | H | H | CH₃O | H |
| 359 | F | CN | General Formula (12) | General Formula (12) | General Formula (12) | H | H | H | H |
| 360 | F | CN | General Formula (12) | General Formula (12) | General Formula (12) | H | H | CH₃ | H |
| 361 | F | CN | General Formula (12) | General Formula (12) | General Formula (12) | H | H | CH₃O | H |
| 362 | General Formula (12) | CN | General Formula (12) | F | F | H | H | H | H |
| 363 | General Formula (12) | CN | General Formula (12) | F | F | H | H | CH₃ | H |
| 364 | General Formula (12) | CN | General Formula (12) | F | F | H | H | CH₃O | H |
| 365 | General Formula (12) | CN | F | General Formula (12) | F | H | H | H | H |
| 366 | General Formula (12) | CN | F | General Formula (12) | F | H | H | CH₃ | H |
| 367 | General Formula (12) | CN | F | General Formula (12) | F | H | H | CH₃O | H |
| 368 | F | CN | General Formula (12) | General Formula (12) | F | H | H | H | H |
| 369 | F | CN | General Formula (12) | General Formula (12) | F | H | H | CH₃ | H |
| 370 | F | CN | General Formula (12) | General Formula (12) | F | H | H | CH₃O | H |
| 371 | General Formula (12) | CN | F | F | General Formula (12) | H | H | H | H |
| 372 | General Formula (12) | CN | F | F | General Formula (12) | H | H | CH₃ | H |
| 373 | General Formula (12) | CN | F | F | General Formula (12) | H | H | CH₃O | H |
| 374 | F | CN | General Formula (12) | F | General Formula (12) | H | H | H | H |
| 375 | F | CN | General Formula (12) | F | General Formula (12) | H | H | CH₃ | H |
| 376 | F | CN | General Formula (12) | F | General Formula (12) | H | H | CH₃O | H |
| 377 | F | CN | F | General Formula (12) | General Formula (12) | H | H | H | H |
| 378 | F | CN | F | General Formula (12) | General Formula (12) | H | H | CH₃ | H |
| 379 | F | CN | F | General Formula (12) | General Formula (12) | H | H | CH₃O | H |
| 380 | General Formula (12) | CN | F | F | F | H | H | H | H |
| 381 | General Formula (12) | CN | F | F | F | H | H | CH₃ | H |
| 382 | General Formula (12) | CN | F | F | F | H | H | CH₃O | H |
| 383 | F | CN | General Formula (12) | F | F | H | H | H | H |
| 384 | F | CN | General Formula (12) | F | F | H | H | CH₃ | H |
| 385 | F | CN | General Formula (12) | F | F | H | H | CH₃O | H |
| 386 | F | CN | F | General Formula (12) | F | H | H | H | H |
| 387 | F | CN | F | General Formula (12) | F | H | H | CH₃ | H |
| 388 | F | CN | F | General Formula (12) | F | H | H | CH₃O | H |
| 389 | General Formula (12) | CN | General Formula (12) | General Formula (12) | OH | H | H | H | H |
| 390 | General Formula (12) | CN | General Formula (12) | General Formula (12) | OH | H | H | CH₃ | H |
| 391 | General Formula (12) | CN | General Formula (12) | General Formula (12) | OH | H | H | CH₃O | H |
| 392 | General Formula (12) | CN | General Formula (12) | OH | General Formula (12) | H | H | H | H |
| 393 | General Formula (12) | CN | General Formula (12) | OH | General Formula (12) | H | H | CH₃ | H |
| 394 | General Formula (12) | CN | General Formula (12) | OH | General Formula (12) | H | H | CH₃O | H |
| 395 | General Formula (12) | CN | General Formula (12) | OH | General Formula (12) | H | H | t-C₄H₉ | H |
| 396 | General Formula (12) | CN | General Formula (12) | OH | General Formula (12) | H | H | Cl | H |
| 397 | General Formula (12) | CN | General Formula (12) | OH | General Formula (12) | H | H | F | H |
| 398 | General Formula (12) | CN | OH | General Formula (12) | General Formula (12) | H | H | H | H |
| 399 | General Formula (12) | CN | OH | General Formula (12) | General Formula (12) | H | H | CH₃ | H |
| 400 | General Formula (12) | CN | OH | General Formula (12) | General Formula (12) | H | H | CH₃O | H |
| 401 | OH | CN | General Formula (12) | General Formula (12) | General Formula (12) | H | H | H | H |
| 402 | OH | CN | General Formula (12) | General Formula (12) | General Formula (12) | H | H | CH₃ | H |
| 403 | OH | CN | General Formula (12) | General Formula (12) | General Formula (12) | H | H | CH₃O | H |
| 404 | General Formula (12) | CN | General Formula (12) | OH | OH | H | H | H | H |
| 405 | General Formula (12) | CN | General Formula (12) | OH | OH | H | H | CH₃ | H |
| 406 | General Formula (12) | CN | General Formula (12) | OH | OH | H | H | CH₃O | H |
| 407 | General Formula (12) | CN | OH | General Formula (12) | OH | H | H | H | H |
| 408 | General Formula (12) | CN | OH | General Formula (12) | OH | H | H | CH₃ | H |
| 409 | General Formula (12) | CN | OH | General Formula (12) | OH | H | H | CH₃O | H |
| 410 | OH | CN | General Formula (12) | General Formula (12) | OH | H | H | H | H |
| 411 | OH | CN | General Formula (12) | General Formula (12) | OH | H | H | CH₃ | H |
| 412 | OH | CN | General Formula (12) | General Formula (12) | OH | H | H | CH₃O | H |
| 413 | General Formula (12) | CN | OH | OH | General Formula (12) | H | H | H | H |
| 414 | General Formula (12) | CN | OH | OH | General Formula (12) | H | H | CH₃ | H |
| 415 | General Formula (12) | CN | OH | OH | General Formula (12) | H | H | CH₃O | H |
| 416 | OH | CN | General Formula (12) | OH | General Formula (12) | H | H | H | H |
| 417 | OH | CN | General Formula (12) | OH | General Formula (12) | H | H | CH₃ | H |
| 418 | OH | CN | General Formula (12) | OH | General Formula (12) | H | H | CH₃O | H |
| 419 | OH | CN | OH | General Formula (12) | General Formula (12) | H | H | H | H |
| 420 | OH | CN | OH | General Formula (12) | General Formula (12) | H | H | CH₃ | H |
| 421 | OH | CN | OH | General Formula (12) | General Formula (12) | H | H | CH₃O | H |
| 422 | General Formula (12) | CN | OH | OH | OH | H | H | H | H |
| 423 | General Formula (12) | CN | OH | OH | OH | H | H | CH₃ | H |
| 424 | General Formula (12) | CN | OH | OH | OH | H | H | CH₃O | H |
| 425 | OH | CN | General Formula (12) | OH | OH | H | H | H | H |
| 426 | OH | CN | General Formula (12) | OH | OH | H | H | CH₃ | H |
| 427 | OH | CN | General Formula (12) | OH | OH | H | H | CH₃O | H |
| 428 | OH | CN | OH | General Formula (12) | OH | H | H | H | H |
| 429 | OH | CN | OH | General Formula (12) | OH | H | H | CH₃ | H |
| 430 | OH | CN | OH | General Formula (12) | OH | H | H | CH₃O | H |
| 431 | OH | CN | OH | OH | General Formula (12) | H | H | H | H |
| 432 | OH | CN | OH | OH | General Formula (12) | H | H | CH₃ | H |
| 433 | OH | CN | OH | OH | General Formula (12) | H | H | CH₃O | H |
| 434 | General Formula (12) | CN | General Formula (12) | Cl | General Formula (12) | H | H | H | H |
| 435 | General Formula (12) | CN | General Formula (12) | Cl | General Formula (12) | H | H | CH₃ | H |
| 436 | General Formula (12) | CN | General Formula (12) | Cl | General Formula (12) | H | H | CH₃O | H |
| 437 | General Formula (12) | CN | General Formula (12) | Cl | General Formula (12) | H | H | t-C₄H₉ | H |
| 438 | General Formula (12) | CN | General Formula (12) | Cl | General Formula (12) | H | H | Cl | H |
| 439 | General Formula (12) | CN | General Formula (12) | Cl | General Formula (12) | H | H | F | H |
| 440 | General Formula (12) | CN | General Formula (12) | F | General Formula (12) | H | H | H | H |
| 441 | General Formula (12) | CN | General Formula (12) | F | General Formula (12) | H | H | CH₃ | H |
| 442 | General Formula (12) | CN | General Formula (12) | F | General Formula (12) | H | H | CH₃O | H |
| 443 | General Formula (12) | CN | General Formula (12) | F | General Formula (12) | H | H | t-C₄H₉ | H |
| 444 | General Formula (12) | CN | General Formula (12) | F | General Formula (12) | H | H | Cl | H |
| 445 | General Formula (12) | CN | General Formula (12) | F | General Formula (12) | H | H | F | H |
| 446 | General Formula (12) | CN | General Formula (12) | CH₃O | General Formula (12) | H | H | H | H |
| 447 | General Formula (12) | CN | General Formula (12) | CH₃O | General Formula (12) | H | H | CH₃ | H |
| 448 | General Formula (12) | CN | General Formula (12) | CH₃O | General Formula (12) | H | H | CH₃O | H |
| 449 | General Formula (12) | CN | General Formula (12) | CH₃O | General Formula (12) | H | H | t-C₄H₉ | H |
| 450 | General Formula (12) | CN | General Formula (12) | CH₃O | General Formula (12) | H | H | Cl | H |
| 451 | General Formula (12) | CN | General Formula (12) | CH₃O | General Formula (12) | H | H | F | H |
| 452 | General Formula (12) | CN | General Formula (12) | C₂H₅O | General Formula (12) | H | H | H | H |
| 453 | General Formula (12) | CN | General Formula (12) | C₂H₅O | General Formula (12) | H | H | CH₃ | H |
| 454 | General Formula (12) | CN | General Formula (12) | C₂H₅O | General Formula (12) | H | H | CH₃O | H |
| 455 | General Formula (12) | CN | General Formula (12) | C₂H₅O | General Formula (12) | H | H | t-C₄H₉ | H |
| 456 | General Formula (12) | CN | General Formula (12) | C₂H₅O | General Formula (12) | H | H | Cl | H |
| 457 | General Formula (12) | CN | General Formula (12) | C₂H₅O | General Formula (12) | H | H | F | H |
| 458 | General Formula (12) | CN | General Formula (12) | C₆H₅O | General Formula (12) | H | H | H | H |
| 459 | General Formula (12) | CN | General Formula (12) | C₆H₅O | General Formula (12) | H | H | CH₃ | H |
| 460 | General Formula (12) | CN | General Formula (12) | C₆H₅O | General Formula (12) | H | H | CH₃O | H |
| 461 | General Formula (12) | CN | General Formula (12) | C₆H₅O | General Formula (12) | H | H | t-C₄H₉ | H |
| 462 | General Formula (12) | CN | General Formula (12) | C₆H₅O | General Formula (12) | H | H | Cl | H |
| 463 | General Formula (12) | CN | General Formula (12) | C₆H₅O | General Formula (12) | H | H | F | H |
| 464 | General Formula (12) | CN | General Formula (12) | Formula (21) | General Formula (12) | H | H | H | H |
| 465 | General Formula (12) | CN | General Formula (12) | Formula (21) | General Formula (12) | H | H | CH₃ | H |
| 466 | General Formula (12) | CN | General Formula (12) | Formula (21) | General Formula (12) | H | H | CH₃O | H |
| 467 | General Formula (12) | CN | General Formula (12) | Formula (21) | General Formula (12) | H | H | t-C₄H₉ | H |
| 468 | General Formula (12) | CN | General Formula (12) | Formula (21) | General Formula (12) | H | H | Cl | H |
| 469 | General Formula (12) | CN | General Formula (12) | Formula (21) | General Formula (12) | H | H | F | H |
| 470 | General Formula (12) | CN | General Formula (12) | Formula (22) | General Formula (12) | H | H | H | H |
| 471 | General Formula (12) | CN | General Formula (12) | Formula (22) | General Formula (12) | H | H | CH₃ | H |
| 472 | General Formula (12) | CN | General Formula (12) | Formula (22) | General Formula (12) | H | H | CH₃O | H |
| 473 | General Formula (12) | CN | General Formula (12) | Formula (22) | General Formula (12) | H | H | t-C₄H₉ | H |
| 474 | General Formula (12) | CN | General Formula (12) | Formula (22) | General Formula (12) | H | H | Cl | H |
| 475 | General Formula (12) | CN | General Formula (12) | Formula (22) | General Formula (12) | H | H | F | H |
| 476 | General Formula (12) | CN | General Formula (12) | Formula (23) | General Formula (12) | H | H | H | H |
| 477 | General Formula (12) | CN | General Formula (12) | Formula (23) | General Formula (12) | H | H | CH₃ | H |
| 478 | General Formula (12) | CN | General Formula (12) | Formula (23) | General Formula (12) | H | H | CH₃O | H |
| 479 | General Formula (12) | CN | General Formula (12) | Formula (23) | General Formula (12) | H | H | t-C₄H₉ | H |
| 480 | General Formula (12) | CN | General Formula (12) | Formula (23) | General Formula (12) | H | H | Cl | H |
| 481 | General Formula (12) | CN | General Formula (12) | Formula (23) | General Formula (12) | H | H | F | H |
| 482 | General Formula (12) | CN | General Formula (12) | Formula (24) | General Formula (12) | H | H | H | H |
| 483 | General Formula (12) | CN | General Formula (12) | Formula (24) | General Formula (12) | H | H | CH₃ | H |
| 484 | General Formula (12) | CN | General Formula (12) | Formula (24) | General Formula (12) | H | H | CH₃O | H |
| 485 | General Formula (12) | CN | General Formula (12) | Formula (24) | General Formula (12) | H | H | t-C₄H₉ | H |
| 486 | General Formula (12) | CN | General Formula (12) | Formula (24) | General Formula (12) | H | H | Cl | H |
| 487 | General Formula (12) | CN | General Formula (12) | Formula (24) | General Formula (12) | H | H | F | H |
| 488 | General Formula (12) | CN | General Formula (12) | General Formula (12) | General Formula (12) | H | C₆H₅ | H | H |
| 489 | General Formula (12) | CN | General Formula (12) | General Formula (12) | General Formula (12) | H | H | C₆H₅ | H |
| 490 | General Formula (12) | CN | General Formula (12) | General Formula (12) | H | H | C₆H₅ | H | H |
| 491 | General Formula (12) | CN | General Formula (12) | General Formula (12) | H | H | H | C₆H₅ | H |
| 492 | General Formula (12) | CN | General Formula (12) | H | General Formula (12) | H | C₆H₅ | H | H |
| 493 | General Formula (12) | CN | General Formula (12) | H | General Formula (12) | H | H | C₆H₅ | H |
| 494 | General Formula (12) | CN | H | General Formula (12) | General Formula (12) | H | C₆H₅ | H | H |
| 495 | General Formula (12) | CN | H | General Formula (12) | General Formula (12) | H | H | C₆H₅ | H |
| 496 | H | CN | General Formula (12) | General Formula (12) | General Formula (12) | H | C₆H₅ | H | H |
| 497 | H | CN | General Formula (12) | General Formula (12) | General Formula (12) | H | H | C₆H₅ | H |
| 498 | General Formula (12) | CN | General Formula (12) | H | H | H | C₆H₅ | H | H |
| 499 | General Formula (12) | CN | General Formula (12) | H | H | H | H | C₆H₅ | H |
| 500-1 | General Formula (12) | CN | H | General Formula (12) | H | H | C₆H₅ | H | H |
| 500-2 | General Formula (12) | CN | H | General Formula (12) | H | H | H | C₆H₅ | H |
| 500-3 | H | CN | General Formula (12) | General Formula (12) | H | H | C₆H₅ | H | H |
| 500-4 | H | CN | General Formula (12) | General Formula (12) | H | H | H | C₆H₅ | H |
| 500-5 | General Formula (12) | CN | H | H | General Formula (12) | H | C₆H₅ | H | H |
| 500-6 | General Formula (12) | CN | H | H | General Formula (12) | H | H | C₆H₅ | H |
| 500-7 | H | CN | General Formula (12) | H | General Formula (12) | H | C₆H₅ | H | H |
| 500-8 | H | CN | General Formula (12) | H | General Formula (12) | H | H | C₆H₅ | H |
| 500-9 | H | CN | H | General Formula (12) | General Formula (12) | H | C₆H₅ | H | H |
| 500-10 | H | CN | H | General Formula (12) | General Formula (12) | H | H | C₆H₅ | H |
| 500-11 | General Formula (12) | CN | H | H | H | H | C₆H₅ | H | H |
| 500-12 | General Formula (12) | CN | H | H | H | H | H | C₆H₅ | H |
| 500-13 | H | CN | General Formula (12) | H | H | H | C₆H₅ | H | H |
| 500-14 | H | CN | General Formula (12) | H | H | H | H | C₆H₅ | H |
| 500-15 | H | CN | H | General Formula (12) | H | H | C₆H₅ | H | H |
| 500-16 | H | CN | H | General Formula (12) | H | H | H | C₆H₅ | H |
| 500-17 | General Formula (12) | CN | General Formula (12) | General Formula (12) | F | H | H | C₆H₅ | H |
| 500-18 | General Formula (12) | CN | General Formula (12) | F | General Formula (12) | H | H | C₆H₅ | H |
| 500-19 | General Formula (12) | CN | F | General Formula (12) | General Formula (12) | H | H | C₆H₅ | H |
| 500-20 | F | CN | General Formula (12) | General Formula (12) | General Formula (12) | H | H | C₆H₅ | H |
| 500-21 | General Formula (12) | CN | General Formula (12) | F | F | H | H | C₆H₅ | H |
| 500-22 | General Formula (12) | CN | F | General Formula (12) | F | H | H | C₆H₅ | H |
| 500-23 | F | CN | General Formula (12) | General Formula (12) | F | H | H | C₆H₅ | H |
| 500-24 | General Formula (12) | CN | F | F | General Formula (12) | H | H | C₆H₅ | H |
| 500-25 | F | CN | General Formula (12) | F | General Formula (12) | H | H | C₆H₅ | H |
| 500-26 | F | CN | F | General Formula (12) | General Formula (12) | H | H | C₆H₅ | H |
| 500-27 | General Formula (12) | CN | F | F | F | H | H | C₆H₅ | H |
| 500-28 | F | CN | General Formula (12) | F | F | H | H | C₆H₅ | H |
| 500-29 | F | CN | F | General Formula (12) | F | H | H | C₆H₅ | H |
| 500-30 | General Formula (12) | CN | General Formula (12) | General Formula (12) | OH | H | H | C₆H₅ | H |
| 500-31 | General Formula (12) | CN | General Formula (12) | OH | General Formula (12) | H | H | C₆H₅ | H |
| 500-32 | General Formula (12) | CN | OH | General Formula (12) | General Formula (12) | H | H | C₆H₅ | H |
| 500-33 | OH | CN | General Formula (12) | General Formula (12) | General Formula (12) | H | H | C₆H₅ | H |
| 500-34 | General Formula (12) | CN | General Formula (12) | OH | OH | H | H | C₆H₅ | H |
| 500-35 | General Formula (12) | CN | OH | General Formula (12) | OH | H | H | C₆H₅ | H |
| 500-36 | OH | CN | General Formula (12) | General Formula (12) | OH | H | H | C₆H₅ | H |
| 500-37 | General Formula (12) | CN | OH | OH | General Formula (12) | H | H | C₆H₅ | H |
| 500-38 | OH | CN | General Formula (12) | OH | General Formula (12) | H | H | C₆H₅ | H |
| 500-39 | OH | CN | OH | General Formula (12) | General Formula (12) | H | H | C₆H₅ | H |
| 500-40 | General Formula (12) | CN | OH | OH | OH | H | H | C₆H₅ | H |
| 500-41 | OH | CN | General Formula (12) | OH | OH | H | H | C₆H₅ | H |
| 500-42 | OH | CN | OH | General Formula (12) | OH | H | H | C₆H₅ | H |
| 500-43 | OH | CN | OH | OH | General Formula (12) | H | H | C₆H₅ | H |
| 500-44 | General Formula (12) | CN | General Formula (12) | Cl | General Formula (12) | H | H | C₆H₅ | H |
| 500-45 | General Formula (12) | CN | General Formula (12) | F | General Formula (12) | H | H | C₆H₅ | H |
| 500-46 | General Formula (12) | CN | General Formula (12) | CH₃O | General Formula (12) | H | H | C₆H₅ | H |
| 500-47 | General Formula (12) | CN | General Formula (12) | C₂H₅O | General Formula (12) | H | H | C₆H₅ | H |
| 500-48 | General Formula (12) | CN | General Formula (12) | C₆H₅O | General Formula (12) | H | H | C₆H₅ | H |
| 500-49 | General Formula (12) | CN | General Formula (12) | Formula (21) | General Formula (12) | H | H | C₆H₅ | H |
| 500-50 | General Formula (12) | CN | General Formula (12) | Formula (22) | General Formula (12) | H | H | C₆H₅ | H |
| 500-51 | General Formula (12) | CN | General Formula (12) | Formula (23) | General Formula (12) | H | H | C₆H₅ | H |
| 500-52 | General Formula (12) | CN | General Formula (12) | Formula (24) | General Formula (12) | H | H | C₆H₅ | H |

TABLE 4

| | | |
|---|---|---|
| Compound | General Formula (1) | General Formula (12) |

| No. | R¹ | R² | R³ | R⁴ | R⁵ | R³¹, R³⁸ | R³², R³⁷ | R³³, R³⁶ | R³⁴, R³⁵ |
|---|---|---|---|---|---|---|---|---|---|
| 501 | CN | General Formula (12) | General Formula (12) | General Formula (12) | General Formula (12) | H | H | H | H |
| 502 | CN | General Formula (12) | General Formula (12) | General Formula (12) | General Formula (12) | H | CH₃ | H | H |
| 503 | CN | General Formula (12) | General Formula (12) | General Formula (12) | General Formula (12) | H | CH₃O | H | H |
| 504 | CN | General Formula (12) | General Formula (12) | General Formula (12) | General Formula (12) | H | H | CH₃ | H |
| 505 | CN | General Formula (12) | General Formula (12) | General Formula (12) | General Formula (12) | H | H | CH₃O | H |
| 506 | CN | General Formula (12) | General Formula (12) | General Formula (12) | General Formula (12) | H | H | t-C₄H₉ | H |
| 507 | CN | General Formula (12) | General Formula (12) | General Formula (12) | General Formula (12) | H | H | Cl | H |
| 508 | CN | General Formula (12) | General Formula (12) | General Formula (12) | General Formula (12) | H | H | F | H |
| 509 | CN | General Formula (12) | General Formula (12) | General Formula (12) | General Formula (12) | H | H | H | CH₃ |
| 510 | CN | General Formula (12) | General Formula (12) | General Formula (12) | General Formula (12) | H | H | H | CH₃O |
| 511 | CN | General Formula (12) | General Formula (12) | General Formula (12) | H | H | H | H | H |
| 512 | CN | General Formula (12) | General Formula (12) | General Formula (12) | H | H | H | CH₃ | H |
| 513 | CN | General Formula (12) | General Formula (12) | General Formula (12) | H | H | H | CH₃O | H |
| 514 | CN | General Formula (12) | General Formula (12) | H | General Formula (12) | H | H | H | H |
| 515 | CN | General Formula (12) | General Formula (12) | H | General Formula (12) | H | H | CH₃ | H |
| 516 | CN | General Formula (12) | General Formula (12) | H | General Formula (12) | H | H | CH₃O | H |
| 517 | CN | General Formula (12) | General Formula (12) | H | H | H | H | H | H |
| 518 | CN | General Formula (12) | General Formula (12) | H | H | H | H | CH₃ | H |
| 519 | CN | General Formula (12) | General Formula (12) | H | H | H | H | CH₃O | H |
| 520 | CN | General Formula (12) | H | General Formula (12) | H | H | H | H | H |
| 521 | CN | General Formula (12) | H | General Formula (12) | H | H | H | CH₃ | H |
| 522 | CN | General Formula (12) | H | General Formula (12) | H | H | H | CH₃O | H |
| 523 | CN | H | General Formula (12) | General Formula (12) | H | H | H | H | H |
| 524 | CN | H | General Formula (12) | General Formula (12) | H | H | H | CH₃ | H |
| 525 | CN | H | General Formula (12) | General Formula (12) | H | H | H | CH₃O | H |
| 526 | CN | General Formula (12) | H | H | General Formula (12) | H | H | H | H |
| 527 | CN | General Formula (12) | H | H | General Formula (12) | H | H | CH₃ | H |
| 528 | CN | General Formula (12) | H | H | General Formula (12) | H | H | CH₃O | H |
| 529 | CN | General Formula (12) | H | H | H | H | H | H | H |
| 530 | CN | General Formula (12) | H | H | H | H | H | CH₃ | H |
| 531 | CN | General Formula (12) | H | H | H | H | H | CH₃O | H |
| 532 | CN | H | General Formula (12) | H | H | H | H | H | H |
| 533 | CN | H | General Formula (12) | H | H | H | H | CH₃ | H |
| 534 | CN | H | General Formula (12) | H | H | H | H | CH₃O | H |
| 535 | CN | General Formula (12) | General Formula (12) | General Formula (12) | F | H | H | H | H |
| 536 | CN | General Formula (12) | General Formula (12) | General Formula (12) | F | H | H | CH₃ | H |
| 537 | CN | General Formula (12) | General Formula (12) | General Formula (12) | F | H | H | CH₃O | H |
| 538 | CN | General Formula (12) | General Formula (12) | F | General Formula (12) | H | H | H | H |
| 539 | CN | General Formula (12) | General Formula (12) | F | General Formula (12) | H | H | CH₃ | H |
| 540 | CN | General Formula (12) | General Formula (12) | F | General Formula (12) | H | H | CH₃O | H |
| 541 | CN | General Formula (12) | General Formula (12) | F | F | H | H | H | H |
| 542 | CN | General Formula (12) | General Formula (12) | F | F | H | H | CH₃ | H |
| 543 | CN | General Formula (12) | General Formula (12) | F | F | H | H | CH₃O | H |
| 544 | CN | General Formula (12) | F | General Formula (12) | F | H | H | H | H |
| 545 | CN | General Formula (12) | F | General Formula (12) | F | H | H | CH₃ | H |
| 546 | CN | General Formula (12) | F | General Formula (12) | F | H | H | CH₃O | H |
| 547 | CN | F | General Formula (12) | General Formula (12) | F | H | H | H | H |
| 548 | CN | F | General Formula (12) | General Formula (12) | F | H | H | CH₃ | H |
| 549 | CN | F | General Formula (12) | General Formula (12) | F | H | H | CH₃O | H |
| 550 | CN | General Formula (12) | F | F | General Formula (12) | H | H | H | H |
| 551 | CN | General Formula (12) | F | F | General Formula (12) | H | H | CH₃ | H |
| 552 | CN | General Formula (12) | F | F | General Formula (12) | H | H | CH₃O | H |
| 553 | CN | General Formula (12) | F | F | F | H | H | H | H |
| 554 | CN | General Formula (12) | F | F | F | H | H | CH₃ | H |
| 555 | CN | General Formula (12) | F | F | F | H | H | CH₃O | H |
| 556 | CN | F | General Formula (12) | F | F | H | H | H | H |
| 557 | CN | F | General Formula (12) | F | F | H | H | CH₃ | H |
| 558 | CN | F | General Formula (12) | F | F | H | H | CH₃O | H |
| 559 | CN | General Formula (12) | General Formula (12) | General Formula (12) | OH | H | H | H | H |
| 560 | CN | General Formula (12) | General Formula (12) | General Formula (12) | OH | H | H | CH₃ | H |
| 561 | CN | General Formula (12) | General Formula (12) | General Formula (12) | OH | H | H | CH₃O | H |
| 562 | CN | General Formula (12) | General Formula (12) | OH | General Formula (12) | H | H | H | H |
| 563 | CN | General Formula (12) | General Formula (12) | OH | General Formula (12) | H | H | CH₃ | H |
| 654 | CN | General Formula (12) | General Formula (12) | OH | General Formula (12) | H | H | CH₃O | H |
| 565 | CN | General Formula (12) | General Formula (12) | OH | General Formula (12) | H | H | Cl | H |
| 566 | CN | General Formula (12) | General Formula (12) | OH | General Formula (12) | H | H | F | H |
| 567 | CN | General Formula (12) | General Formula (12) | OH | OH | H | H | H | H |
| 568 | CN | General Formula (12) | General Formula (12) | OH | OH | H | H | CH₃ | H |
| 569 | CN | General Formula (12) | General Formula (12) | OH | OH | H | H | CH₃O | H |
| 570 | CN | General Formula (12) | OH | General Formula (12) | OH | H | H | H | H |
| 571 | CN | General Formula (12) | OH | General Formula (12) | OH | H | H | CH₃ | H |
| 572 | CN | General Formula (12) | OH | General Formula (12) | OH | H | H | CH₃O | H |
| 573 | CN | OH | General Formula (12) | General Formula (12) | OH | H | H | H | H |
| 574 | CN | OH | General Formula (12) | General Formula (12) | OH | H | H | CH₃ | H |
| 575 | CN | OH | General Formula (12) | General Formula (12) | OH | H | H | CH₃O | H |
| 576 | CN | General Formula (12) | OH | OH | General Formula (12) | H | H | H | H |
| 577 | CN | General Formula (12) | OH | OH | General Formula (12) | H | H | CH₃ | H |
| 578 | CN | General Formula (12) | OH | OH | General Formula (12) | H | H | CH₃O | H |
| 579 | CN | General Formula (12) | OH | OH | OH | H | H | H | H |
| 580 | CN | General Formula (12) | OH | OH | OH | H | H | CH₃ | H |
| 581 | CN | General Formula (12) | OH | OH | OH | H | H | CH₃O | H |
| 582 | CN | OH | General Formula (12) | OH | OH | H | H | H | H |
| 583 | CN | OH | General Formula (12) | OH | OH | H | H | CH₃ | H |
| 584 | CN | OH | General Formula (12) | OH | OH | H | H | CH₃O | H |
| 585 | CN | General Formula (12) | General Formula (12) | Cl | General Formula (12) | H | H | H | H |
| 586 | CN | General Formula (12) | General Formula (12) | Cl | General Formula (12) | H | H | CH₃ | H |
| 587 | CN | General Formula (12) | General Formula (12) | Cl | General Formula (12) | H | H | CH₃O | H |
| 588 | CN | General Formula (12) | General Formula (12) | Cl | General Formula (12) | H | H | t-C₄H₉ | H |
| 589 | CN | General Formula (12) | General Formula (12) | Cl | General Formula (12) | H | H | Cl | H |
| 590 | CN | General Formula (12) | General Formula (12) | Cl | General Formula (12) | H | H | F | H |
| 591 | CN | General Formula (12) | General Formula (12) | F | General Formula (12) | H | H | H | H |
| 592 | CN | General Formula (12) | General Formula (12) | F | General Formula (12) | H | H | CH₃ | H |
| 593 | CN | General Formula (12) | General Formula (12) | F | General Formula (12) | H | H | CH₃O | H |
| 594 | CN | General Formula (12) | General Formula (12) | F | General Formula (12) | H | H | t-C₄H₉ | H |
| 595 | CN | General Formula (12) | General Formula (12) | F | General Formula (12) | H | H | Cl | H |
| 596 | CN | General Formula (12) | General Formula (12) | F | General Formula (12) | H | H | F | H |
| 597 | CN | General Formula (12) | General Formula (12) | CH₃O | General Formula (12) | H | H | H | H |
| 598 | CN | General Formula (12) | General Formula (12) | CH₃O | General Formula (12) | H | H | CH₃ | H |
| 599 | CN | General Formula (12) | General Formula (12) | CH₃O | General Formula (12) | H | H | CH₃O | H |
| 600 | CN | General Formula (12) | General Formula (12) | CH₃O | General Formula (12) | H | H | t-C₄H₉ | H |
| 601 | CN | General Formula (12) | General Formula (12) | CH₃O | General Formula (12) | H | H | Cl | H |
| 602 | CN | General Formula (12) | General Formula (12) | CH₃O | General Formula (12) | H | H | F | H |
| 603 | CN | General Formula (12) | General Formula (12) | C₂H₅O | General Formula (12) | H | H | H | H |
| 604 | CN | General Formula (12) | General Formula (12) | C₂H₅O | General Formula (12) | H | H | CH₃ | H |
| 605 | CN | General Formula (12) | General Formula (12) | C₂H₅O | General Formula (12) | H | H | CH₃O | H |
| 606 | CN | General Formula (12) | General Formula (12) | C₂H₅O | General Formula (12) | H | H | t-C₄H₉ | H |
| 607 | CN | General Formula (12) | General Formula (12) | C₂H₅O | General Formula (12) | H | H | Cl | H |
| 608 | CN | General Formula (12) | General Formula (12) | C₂H₅O | General Formula (12) | H | H | F | H |
| 609 | CN | General Formula (12) | General Formula (12) | C₆H₅O | General Formula (12) | H | H | H | H |
| 610 | CN | General Formula (12) | General Formula (12) | C₆H₅O | General Formula (12) | H | H | CH₃ | H |
| 611 | CN | General Formula (12) | General Formula (12) | C₆H₅O | General Formula (12) | H | H | CH₃O | H |
| 612 | CN | General Formula (12) | General Formula (12) | C₆H₅O | General Formula (12) | H | H | t-C₄H₉ | H |
| 613 | CN | General Formula (12) | General Formula (12) | C₆H₅O | General Formula (12) | H | H | Cl | H |
| 614 | CN | General Formula (12) | General Formula (12) | C₆H₅O | General Formula (12) | H | H | F | H |
| 615 | CN | General Formula (12) | General Formula (12) | Formula (21) | General Formula (12) | H | H | H | H |
| 616 | CN | General Formula (12) | General Formula (12) | Formula (21) | General Formula (12) | H | H | CH₃ | H |
| 617 | CN | General Formula (12) | General Formula (12) | Formula (21) | General Formula (12) | H | H | CH₃O | H |
| 618 | CN | General Formula (12) | General Formula (12) | Formula (21) | General Formula (12) | H | H | t-C₄H₉ | H |
| 619 | CN | General Formula (12) | General Formula (12) | Formula (21) | General Formula (12) | H | H | Cl | H |
| 620 | CN | General Formula (12) | General Formula (12) | Formula (21) | General Formula (12) | H | H | F | H |
| 621 | CN | General Formula (12) | General Formula (12) | Formula (22) | General Formula (12) | H | H | H | H |
| 622 | CN | General Formula (12) | General Formula (12) | Formula (22) | General Formula (12) | H | H | CH₃ | H |
| 623 | CN | General Formula (12) | General Formula (12) | Formula (22) | General Formula (12) | H | H | CH₃O | H |
| 624 | CN | General Formula (12) | General Formula (12) | Formula (22) | General Formula (12) | H | H | t-C₄H₉ | H |
| 625 | CN | General Formula (12) | General Formula (12) | Formula (22) | General Formula (12) | H | H | Cl | H |
| 626 | CN | General Formula (12) | General Formula (12) | Formula (22) | General Formula (12) | H | H | F | H |
| 627 | CN | General Formula (12) | General Formula (12) | Formula (23) | General Formula (12) | H | H | H | H |
| 628 | CN | General Formula (12) | General Formula (12) | Formula (23) | General Formula (12) | H | H | CH₃ | H |
| 629 | CN | General Formula (12) | General Formula (12) | Formula (23) | General Formula (12) | H | H | CH₃O | H |
| 630 | CN | General Formula (12) | General Formula (12) | Formula (23) | General Formula (12) | H | H | t-C₄H₉ | H |
| 631 | CN | General Formula (12) | General Formula (12) | Formula (23) | General Formula (12) | H | H | Cl | H |
| 632 | CN | General Formula (12) | General Formula (12) | Formula (23) | General Formula (12) | H | H | F | H |
| 633 | CN | General Formula (12) | General Formula (12) | Formula (24) | General Formula (12) | H | H | H | H |
| 634 | CN | General Formula (12) | General Formula (12) | Formula (24) | General Formula (12) | H | H | CH₃ | H |
| 635 | CN | General Formula (12) | General Formula (12) | Formula (24) | General Formula (12) | H | H | CH₃O | H |
| 636 | CN | General Formula (12) | General Formula (12) | Formula (24) | General Formula (12) | H | H | t-C₄H₉ | H |
| 637 | CN | General Formula (12) | General Formula (12) | Formula (24) | General Formula (12) | H | H | Cl | H |
| 638 | CN | General Formula (12) | General Formula (12) | Formula (24) | General Formula (12) | H | H | F | H |
| 639 | CN | General Formula (12) | General Formula (12) | General Formula (12) | General Formula (12) | H | C₆H₅ | H | H |
| 640 | CN | General Formula (12) | General Formula (12) | General Formula (12) | General Formula (12) | H | H | C₆H₅ | H |
| 641 | CN | General Formula (12) | General Formula (12) | General Formula (12) | H | H | C₆H₅ | H | H |
| 642 | CN | General Formula (12) | General Formula (12) | General Formula (12) | H | H | H | C₆H₅ | H |
| 643 | CN | General Formula (12) | General Formula (12) | H | General Formula (12) | H | C₆H₅ | H | H |
| 644 | CN | General Formula (12) | General Formula (12) | H | General Formula (12) | H | H | C₆H₅ | H |
| 645 | CN | General Formula (12) | General Formula (12) | H | H | H | C₆H₅ | H | H |
| 646 | CN | General Formula (12) | General Formula (12) | H | H | H | H | C₆H₅ | H |
| 647 | CN | General Formula (12) | H | General Formula (12) | H | H | C₆H₅ | H | H |
| 648 | CN | General Formula (12) | H | General Formula (12) | H | H | H | C₆H₅ | H |
| 649 | CN | H | General Formula (12) | General Formula (12) | H | H | C₆H₅ | H | H |
| 650 | CN | H | General Formula (12) | General Formula (12) | H | H | H | C₆H₅ | H |
| 651 | CN | H | H | General Formula (12) | General Formula (12) | H | C₆H₅ | H | H |
| 652 | CN | H | H | General Formula (12) | General Formula (12) | H | H | C₆H₅ | H |
| 653 | CN | General Formula (12) | H | H | H | H | C₆H₅ | H | H |
| 654 | CN | General Formula (12) | H | H | H | H | H | C₆H₅ | H |
| 655 | CN | H | General Formula (12) | H | H | H | C₆H₅ | H | H |
| 656 | CN | H | General Formula (12) | H | H | H | H | C₆H₅ | H |
| 657 | CN | General Formula (12) | General Formula (12) | General Formula (12) | F | H | H | C₆H₅ | H |
| 658 | CN | General Formula (12) | General Formula (12) | F | General Formula (12) | H | H | C₆H₅ | H |
| 659 | CN | General Formula (12) | General Formula (12) | F | F | H | H | C₆H₅ | H |
| 660 | CN | General Formula (12) | F | General Formula (12) | F | H | H | C₆H₅ | H |
| 661 | CN | F | General Formula (12) | General Formula (12) | F | H | H | C₆H₅ | H |
| 662 | CN | F | F | General Formula (12) | General Formula (12) | H | H | C₆H₅ | H |
| 663 | CN | General Formula (12) | F | F | F | H | H | C₆H₅ | H |
| 664 | CN | F | General Formula (12) | F | F | H | H | C₆H₅ | H |
| 665 | CN | General Formula (12) | General Formula (12) | General Formula (12) | OH | H | H | C₆H₅ | H |
| 666 | CN | General Formula (12) | General Formula (12) | OH | General Formula (12) | H | H | C₆H₅ | H |
| 667 | CN | General Formula (12) | General Formula (12) | OH | OH | H | H | C₆H₅ | H |
| 668 | CN | General Formula (12) | OH | General Formula (12) | OH | H | H | C₆H₅ | H |
| 669 | CN | OH | General Formula (12) | General Formula (12) | OH | H | H | C₆H₅ | H |
| 670 | CN | OH | OH | General Formula (12) | General Formula (12) | H | H | C₆H₅ | H |
| 671 | CN | General Formula (12) | OH | OH | OH | H | H | C₆H₅ | H |
| 672 | CN | OH | General Formula (12) | OH | OH | H | H | C₆H₅ | H |
| 673 | CN | General Formula (12) | General Formula (12) | Cl | General Formula (12) | H | H | C₆H₅ | H |
| 674 | CN | General Formula (12) | General Formula (12) | F | General Formula (12) | H | H | C₆H₅ | H |
| 675 | CN | General Formula (12) | General Formula (12) | CH₃O | General Formula (12) | H | H | C₆H₅ | H |
| 676 | CN | General Formula (12) | General Formula (12) | C₂H₅O | General Formula (12) | H | H | C₆H₅ | H |
| 677 | CN | General Formula (12) | General Formula (12) | C₆H₅O | General Formula (12) | H | H | C₆H₅ | H |
| 678 | CN | General Formula (12) | General Formula (12) | Formula (21) | General Formula (12) | H | H | C₆H₅ | H |
| 679 | CN | General Formula (12) | General Formula (12) | Formula (22) | General Formula (12) | H | H | C₆H₅ | H |
| 680 | CN | General Formula (12) | General Formula (12) | Formula (23) | General Formula (12) | H | H | C₆H₅ | H |
| 681 | CN | General Formula (12) | General Formula (12) | Formula (24) | General Formula (12) | H | H | C₆H₅ | H |

TABLE 5

| Compound | General Formula (1) | | | | | General Formula (13) | | | | | |
|---|---|---|---|---|---|---|---|---|---|---|---|
| No. | R¹ | R² | R³ | R⁴ | R⁵ | R⁴¹ | R⁴² | R⁴³ | R⁴⁴ | R⁴⁵ | R⁴⁶ |
| 701 | General Formula (13) | General Formula (13) | CN | General Formula (13) | General Formula (13) | H | H | H | H | H | H |
| 702 | General Formula (13) | General Formula (13) | CN | General Formula (13) | General Formula (13) | H | CH₃ | H | H | H | H |
| 703 | General Formula (13) | General Formula (13) | CN | General Formula (13) | General Formula (13) | H | CH₃O | H | H | H | H |
| 704 | General Formula (13) | General Formula (13) | CN | General Formula (13) | General Formula (13) | H | H | CH₃ | H | H | H |
| 705 | General Formula (13) | General Formula (13) | CN | General Formula (13) | General Formula (13) | H | H | CH₃O | H | H | H |
| 706 | General Formula (13) | General Formula (13) | CN | General Formula (13) | General Formula (13) | H | H | t-C₄H₉ | H | H | H |
| 707 | General Formula (13) | General Formula (13) | CN | General Formula (13) | General Formula (13) | H | H | Cl | H | H | H |
| 708 | General Formula (13) | General Formula (13) | CN | General Formula (13) | General Formula (13) | H | H | F | H | H | H |
| 709 | General Formula (13) | General Formula (13) | CN | General Formula (13) | General Formula (13) | H | H | H | CH₃ | H | H |
| 710 | General Formula (13) | General Formula (13) | CN | General Formula (13) | General Formula (13) | H | H | H | CH₃O | H | H |
| 711 | General Formula (13) | General Formula (13) | CN | General Formula (13) | General Formula (13) | H | H | H | H | CH₃ | H |
| 712 | General Formula (13) | General Formula (13) | CN | General Formula (13) | General Formula (13) | H | H | H | H | CH₃O | H |
| 713 | General Formula (13) | General Formula (13) | CN | General Formula (13) | General Formula (13) | H | H | H | H | t-C₄H₉ | H |
| 714 | General Formula (13) | General Formula (13) | CN | General Formula (13) | General Formula (13) | H | H | H | H | Cl | H |
| 715 | General Formula (13) | General Formula (13) | CN | General Formula (13) | General Formula (13) | H | H | H | H | F | H |
| 716 | General Formula (13) | General Formula (13) | CN | General Formula (13) | General Formula (13) | H | H | H | H | C₆H₅ | H |
| 717 | General Formula (13) | General Formula (13) | CN | General Formula (13) | General Formula (13) | H | H | H | H | p-CH₃C₆H₄ | H |
| 718 | General Formula (13) | General Formula (13) | CN | General Formula (13) | General Formula (13) | H | H | H | H | 2,4,6-(CH₃)₃C₆H₂ | H |
| 719 | General Formula (13) | General Formula (13) | CN | General Formula (13) | General Formula (13) | H | H | H | H | p-CH₃OC₆H₄ | H |
| 720 | General Formula (13) | General Formula (13) | CN | General Formula (13) | General Formula (13) | H | H | H | H | p-(CH₃)₂NC₆H₄ | H |
| 721 | General Formula (13) | General Formula (13) | CN | General Formula (13) | General Formula (13) | H | H | H | H | p-FC₆H₄ | H |
| 722 | General Formula (13) | General Formula (13) | CN | General Formula (13) | General Formula (13) | H | H | H | H | p-CNC₆H₄ | H |
| 723 | General Formula (13) | General Formula (13) | CN | General Formula (13) | General Formula (13) | H | H | H | H | H | CH₃ |
| 724 | General Formula (13) | General Formula (13) | CN | General Formula (13) | General Formula (13) | H | H | H | H | H | CH₃O |
| 725 | General Formula (13) | General Formula (13) | CN | General Formula (13) | General Formula (13) | H | H | H | H | H | t-C₄H₉ |
| 726 | General Formula (13) | General Formula (13) | CN | General Formula (13) | General Formula (13) | H | H | H | H | H | Cl |
| 727 | General Formula (13) | General Formula (13) | CN | General Formula (13) | General Formula (13) | H | H | H | H | H | F |
| 728 | General Formula (13) | General Formula (13) | CN | General Formula (13) | General Formula (13) | H | H | H | H | H | C₆H₅ |
| 729 | General Formula (13) | General Formula (13) | CN | General Formula (13) | General Formula (13) | H | H | H | H | H | p-CH₃C₆H₄ |
| 730 | General Formula (13) | General Formula (13) | CN | General Formula (13) | General Formula (13) | H | H | H | H | H | 2,4,6-(CH₃)₃C₆H₂ |
| 731 | General Formula (13) | General Formula (13) | CN | General Formula (13) | General Formula (13) | H | H | H | H | H | p-CH₃OC₆H₄ |
| 732 | General Formula (13) | General Formula (13) | CN | General Formula (13) | General Formula (13) | H | H | H | H | H | p-(CH₃)₂NC₆H₄ |
| 733 | General Formula (13) | General Formula (13) | CN | General Formula (13) | General Formula (13) | H | H | H | H | H | p-FC₆H₄ |
| 734 | General Formula (13) | General Formula (13) | CN | General Formula (13) | General Formula (13) | H | H | H | H | H | p-CNC₆H₄ |
| 735 | General Formula (13) | General Formula (13) | CN | General Formula (13) | H | H | H | H | H | H | H |
| 736 | General Formula (13) | General Formula (13) | CN | H | General Formula (13) | H | H | H | H | H | H |
| 737 | General Formula (13) | General Formula (13) | CN | H | H | H | H | H | H | H | H |
| 738 | General Formula (13) | H | CN | General Formula (13) | H | H | H | H | H | H | H |
| 739 | H | General Formula (13) | CN | General Formula (13) | H | H | H | H | H | H | H |
| 740 | General Formula (13) | H | CN | General Formula (13) | H | H | H | H | H | H | H |
| 741 | General Formula (13) | General Formula (13) | CN | General Formula (13) | F | H | H | H | H | H | H |
| 742 | General Formula (13) | General Formula (13) | CN | F | General Formula (13) | H | H | H | H | H | H |
| 743 | General Formula (13) | General Formula (13) | CN | F | F | H | H | H | H | H | H |
| 744 | General Formula (13) | F | CN | General Formula (13) | F | H | H | H | H | H | H |
| 745 | F | General Formula (13) | CN | General Formula (13) | F | H | H | H | H | H | H |
| 746 | General Formula (13) | F | CN | F | F | H | H | H | H | H | H |
| 747 | General Formula (13) | General Formula (13) | CN | General Formula (13) | OH | H | H | H | H | H | H |
| 748 | General Formula (13) | General Formula (13) | CN | OH | General Formula (13) | H | H | H | H | H | H |
| 749 | General Formula (13) | General Formula (13) | CN | OH | OH | H | H | H | H | H | H |
| 750 | General Formula (13) | OH | CN | General Formula (13) | OH | H | H | H | H | H | H |
| 751 | OH | General Formula (13) | CN | General Formula (13) | OH | H | H | H | H | H | H |
| 752 | General Formula (13) | OH | CN | OH | OH | H | H | H | H | H | H |
| 753 | General Formula (13) | General Formula (13) | CN | Cl | General Formula (13) | H | H | H | H | H | H |
| 754 | General Formula (13) | General Formula (13) | CN | Cl | General Formula (13) | H | H | CH₃ | H | H | H |
| 755 | General Formula (13) | General Formula (13) | CN | Cl | General Formula (13) | H | H | CH₃O | H | H | H |
| 756 | General Formula (13) | General Formula (13) | CN | Cl | General Formula (13) | H | H | t-C₄H₉ | H | H | H |
| 757 | General Formula (13) | General Formula (13) | CN | Cl | General Formula (13) | H | H | Cl | H | H | H |
| 758 | General Formula (13) | General Formula (13) | CN | Cl | General Formula (13) | H | H | F | H | H | H |
| 759 | General Formula (13) | General Formula (13) | CN | F | General Formula (13) | H | H | H | H | H | H |
| 760 | General Formula (13) | General Formula (13) | CN | F | General Formula (13) | H | H | CH₃ | H | H | H |
| 761 | General Formula (13) | General Formula (13) | CN | F | General Formula (13) | H | H | CH₃O | H | H | H |
| 762 | General Formula (13) | General Formula (13) | CN | F | General Formula (13) | H | H | t-C₄H₉ | H | H | H |
| 763 | General Formula (13) | General Formula (13) | CN | F | General Formula (13) | H | H | Cl | H | H | H |
| 764 | General Formula (13) | General Formula (13) | CN | F | General Formula (13) | H | H | F | H | H | H |
| 765 | General Formula (13) | General Formula (13) | CN | CH₃O | General Formula (13) | H | H | H | H | H | H |
| 766 | General Formula (13) | General Formula (13) | CN | CH₃O | General Formula (13) | H | H | CH₃ | H | H | H |
| 767 | General Formula (13) | General Formula (13) | CN | CH₃O | General Formula (13) | H | H | CH₃O | H | H | H |
| 768 | General Formula (13) | General Formula (13) | CN | CH₃O | General Formula (13) | H | H | t-C₄H₉ | H | H | H |
| 769 | General Formula (13) | General Formula (13) | CN | CH₃O | General Formula (13) | H | H | Cl | H | H | H |
| 770 | General Formula (13) | General Formula (13) | CN | CH₃O | General Formula (13) | H | H | F | H | H | H |
| 771 | General Formula (13) | General Formula (13) | CN | C₂H₅O | General Formula (13) | H | H | H | H | H | H |
| 772 | General Formula (13) | General Formula (13) | CN | C₂H₅O | General Formula (13) | H | H | CH₃ | H | H | H |
| 773 | General Formula (13) | General Formula (13) | CN | C₂H₅O | General Formula (13) | H | H | CH₃O | H | H | H |
| 774 | General Formula (13) | General Formula (13) | CN | C₂H₅O | General Formula (13) | H | H | t-C₄H₉ | H | H | H |
| 775 | General Formula (13) | General Formula (13) | CN | C₂H₅O | General Formula (13) | H | H | Cl | H | H | H |
| 776 | General Formula (13) | General Formula (13) | CN | C₂H₅O | General Formula (13) | H | H | F | H | H | H |
| 777 | General Formula (13) | General Formula (13) | CN | C₆H₅O | General Formula (13) | H | H | H | H | H | H |
| 778 | General Formula (13) | General Formula (13) | CN | C₆H₅O | General Formula (13) | H | H | CH₃ | H | H | H |
| 779 | General Formula (13) | General Formula (13) | CN | C₆H₅O | General Formula (13) | H | H | CH₃O | H | H | H |
| 780 | General Formula (13) | General Formula (13) | CN | C₆H₅O | General Formula (13) | H | H | t-C₄H₉ | H | H | H |
| 781 | General Formula (13) | General Formula (13) | CN | C₆H₅O | General Formula (13) | H | H | Cl | H | H | H |
| 782 | General Formula (13) | General Formula (13) | CN | C₆H₅O | General Formula (13) | H | H | F | H | H | H |
| 783 | General Formula (13) | General Formula (13) | CN | Formula (21) | General Formula (13) | H | H | H | H | H | H |
| 784 | General Formula (13) | General Formula (13) | CN | Formula (21) | General Formula (13) | H | H | CH₃ | H | H | H |
| 785 | General Formula (13) | General Formula (13) | CN | Formula (21) | General Formula (13) | H | H | CH₃O | H | H | H |
| 786 | General Formula (13) | General Formula (13) | CN | Formula (21) | General Formula (13) | H | H | t-C₄H₉ | H | H | H |
| 787 | General Formula (13) | General Formula (13) | CN | Formula (21) | General Formula (13) | H | H | Cl | H | H | H |
| 788 | General Formula (13) | General Formula (13) | CN | Formula (21) | General Formula (13) | H | H | F | H | H | H |
| 789 | General Formula (13) | General Formula (13) | CN | Formula (22) | General Formula (13) | H | H | H | H | H | H |
| 790 | General Formula (13) | General Formula (13) | CN | Formula (22) | General Formula (13) | H | H | CH₃ | H | H | H |
| 791 | General Formula (13) | General Formula (13) | CN | Formula (22) | General Formula (13) | H | H | CH₃O | H | H | H |
| 792 | General Formula (13) | General Formula (13) | CN | Formula (22) | General Formula (13) | H | H | t-C₄H₉ | H | H | H |
| 793 | General Formula (13) | General Formula (13) | CN | Formula (22) | General Formula (13) | H | H | Cl | H | H | H |
| 794 | General Formula (13) | General Formula (13) | CN | Formula (22) | General Formula (13) | H | H | F | H | H | H |
| 795 | General Formula (13) | General Formula (13) | CN | Formula (23) | General Formula (13) | H | H | H | H | H | H |
| 796 | General Formula (13) | General Formula (13) | CN | Formula (23) | General Formula (13) | H | H | CH₃ | H | H | H |
| 797 | General Formula (13) | General Formula (13) | CN | Formula (23) | General Formula (13) | H | H | CH₃O | H | H | H |
| 798 | General Formula (13) | General Formula (13) | CN | Formula (23) | General Formula (13) | H | H | t-C₄H₉ | H | H | H |
| 799 | General Formula (13) | General Formula (13) | CN | Formula (23) | General Formula (13) | H | H | Cl | H | H | H |
| 800 | General Formula (13) | General Formula (13) | CN | Formula (23) | General Formula (13) | H | H | F | H | H | H |
| 801 | General Formula (13) | General Formula (13) | CN | Formula (24) | General Formula (13) | H | H | H | H | H | H |
| 802 | General Formula (13) | General Formula (13) | CN | Formula (24) | General Formula (13) | H | H | CH₃ | H | H | H |
| 803 | General Formula (13) | General Formula (13) | CN | Formula (24) | General Formula (13) | H | H | CH₃O | H | H | H |
| 804 | General Formula (13) | General Formula (13) | CN | Formula (24) | General Formula (13) | H | H | t-C₄H₉ | H | H | H |
| 805 | General Formula (13) | General Formula (13) | CN | Formula (24) | General Formula (13) | H | H | Cl | H | H | H |
| 806 | General Formula (13) | General Formula (13) | CN | Formula (24) | General Formula (13) | H | H | F | H | H | H |

TABLE 6

| | | | | | | General Formula (14) | | | | | | | |
|---|---|---|---|---|---|---|---|---|---|---|---|---|---|
| Compound | General Formula (1) | | | | | | | | | | | | R⁵¹, R⁵⁶, R⁵⁸, |
| No. | R¹ | R² | R³ | R⁴ | R⁵ | R⁵² | R⁵³ | R⁵⁴ | R⁵⁵ | R⁵⁷ | R⁵⁹ | R⁶¹ | R⁶⁰, R⁶² |
| 901 | General Formula (14) | General Formula (14) | CN | General Formula (14) | General Formula (14) | H | H | H | H | H | H | H | H |
| 902 | General Formula (14) | General Formula (14) | CN | General Formula (14) | General Formula (14) | CH₃ | H | H | H | H | H | H | H |
| 903 | General Formula (14) | General Formula (14) | CN | General Formula (14) | General Formula (14) | CH₃O | H | H | H | H | H | H | H |
| 904 | General Formula (14) | General Formula (14) | CN | General Formula (14) | General Formula (14) | H | CH₃ | H | H | H | H | H | H |
| 905 | General Formula (14) | General Formula (14) | CN | General Formula (14) | General Formula (14) | H | CH₃O | H | H | H | H | H | H |
| 906 | General Formula (14) | General Formula (14) | CN | General Formula (14) | General Formula (14) | H | t-C₄H₉ | H | H | H | H | H | H |
| 907 | General Formula (14) | General Formula (14) | CN | General Formula (14) | General Formula (14) | H | Cl | H | H | H | H | H | H |
| 908 | General Formula (14) | General Formula (14) | CN | General Formula (14) | General Formula (14) | H | F | H | H | H | H | H | H |
| 909 | General Formula (14) | General Formula (14) | CN | General Formula (14) | General Formula (14) | H | H | CH₃ | H | H | H | H | H |
| 910 | General Formula (14) | General Formula (14) | CN | General Formula (14) | General Formula (14) | H | H | CH₃O | H | H | H | H | H |
| 911 | General Formula (14) | General Formula (14) | CN | General Formula (14) | General Formula (14) | H | H | H | CH₃ | H | H | H | H |
| 912 | General Formula (14) | General Formula (14) | CN | General Formula (14) | General Formula (14) | H | H | H | CH₃O | H | H | H | H |
| 913 | General Formula (14) | General Formula (14) | CN | General Formula (14) | General Formula (14) | H | H | H | H | CH₃ | H | H | H |
| 914 | General Formula (14) | General Formula (14) | CN | General Formula (14) | General Formula (14) | H | H | H | H | CH₃O | H | H | H |
| 915 | General Formula (14) | General Formula (14) | CN | General Formula (14) | General Formula (14) | H | H | H | H | H | CH₃ | H | H |
| 916 | General Formula (14) | General Formula (14) | CN | General Formula (14) | General Formula (14) | H | H | H | H | H | CH₃O | H | H |
| 917 | General Formula (14) | General Formula (14) | CN | General Formula (14) | General Formula (14) | H | H | H | H | H | H | CH₃ | H |
| 918 | General Formula (14) | General Formula (14) | CN | General Formula (14) | General Formula (14) | H | H | H | H | H | H | CH₃O | H |
| 919 | General Formula (14) | General Formula (14) | CN | General Formula (14) | H | H | H | H | H | H | H | H | H |
| 920 | General Formula (14) | General Formula (14) | CN | H | General Formula (14) | H | H | H | H | H | H | H | H |
| 921 | General Formula (14) | General Formula (14) | CN | H | H | H | H | H | H | H | H | H | H |
| 922 | General Formula (14) | H | CN | General Formula (14) | H | H | H | H | H | H | H | H | H |
| 923 | H | General Formula (14) | CN | General Formula (14) | H | H | H | H | H | H | H | H | H |
| 924 | General Formula (14) | H | CN | H | H | H | H | H | H | H | H | H | H |
| 925 | General Formula (14) | General Formula (14) | CN | General Formula (14) | F | H | H | H | H | H | H | H | H |
| 926 | General Formula (14) | General Formula (14) | CN | F | General Formula (14) | H | H | H | H | H | H | H | H |
| 927 | General Formula (14) | General Formula (14) | CN | F | F | H | H | H | H | H | H | H | H |
| 928 | General Formula (14) | F | CN | General Formula (14) | F | H | H | H | H | H | H | H | H |
| 929 | F | General Formula (14) | CN | General Formula (14) | F | H | H | H | H | H | H | H | H |
| 930 | General Formula (14) | F | CN | F | F | H | H | H | H | H | H | H | H |
| 931 | General Formula (14) | General Formula (14) | CN | General Formula (14) | OH | H | H | H | H | H | H | H | H |
| 932 | General Formula (14) | General Formula (14) | CN | OH | General Formula (14) | H | H | H | H | H | H | H | H |
| 933 | General Formula (14) | General Formula (14) | CN | OH | OH | H | H | H | H | H | H | H | H |
| 934 | General Formula (14) | OH | CN | General Formula (14) | OH | H | H | H | H | H | H | H | H |
| 935 | OH | General Formula (14) | CN | General Formula (14) | OH | H | H | H | H | H | H | H | H |
| 936 | General Formula (14) | OH | CN | OH | OH | H | H | H | H | H | H | H | H |
| 937 | General Formula (14) | General Formula (14) | CN | Cl | General Formula (14) | H | H | H | H | H | H | H | H |
| 938 | General Formula (14) | General Formula (14) | CN | Cl | General Formula (14) | H | CH₃ | H | H | H | H | H | H |
| 939 | General Formula (14) | General Formula (14) | CN | Cl | General Formula (14) | H | CH₃O | H | H | H | H | H | H |
| 940 | General Formula (14) | General Formula (14) | CN | Cl | General Formula (14) | H | t-C₄H₉ | H | H | H | H | H | H |
| 941 | General Formula (14) | General Formula (14) | CN | Cl | General Formula (14) | H | Cl | H | H | H | H | H | H |
| 942 | General Formula (14) | General Formula (14) | CN | Cl | General Formula (14) | H | F | H | H | H | H | H | H |
| 943 | General Formula (14) | General Formula (14) | CN | F | General Formula (14) | H | H | H | H | H | H | H | H |
| 944 | General Formula (14) | General Formula (14) | CN | F | General Formula (14) | H | CH₃ | H | H | H | H | H | H |
| 945 | General Formula (14) | General Formula (14) | CN | F | General Formula (14) | H | CH₃O | H | H | H | H | H | H |
| 946 | General Formula (14) | General Formula (14) | CN | F | General Formula (14) | H | t-C₄H₉ | H | H | H | H | H | H |
| 947 | General Formula (14) | General Formula (14) | CN | F | General Formula (14) | H | Cl | H | H | H | H | H | H |
| 948 | General Formula (14) | General Formula (14) | CN | F | General Formula (14) | H | F | H | H | H | H | H | H |
| 949 | General Formula (14) | General Formula (14) | CN | CH₃O | General Formula (14) | H | H | H | H | H | H | H | H |
| 950 | General Formula (14) | General Formula (14) | CN | CH₃O | General Formula (14) | H | CH₃ | H | H | H | H | H | H |
| 951 | General Formula (14) | General Formula (14) | CN | CH₃O | General Formula (14) | H | CH₃O | H | H | H | H | H | H |
| 952 | General Formula (14) | General Formula (14) | CN | CH₃O | General Formula (14) | H | t-C₄H₉ | H | H | H | H | H | H |
| 953 | General Formula (14) | General Formula (14) | CN | CH₃O | General Formula (14) | H | Cl | H | H | H | H | H | H |
| 954 | General Formula (14) | General Formula (14) | CN | CH₃O | General Formula (14) | H | F | H | H | H | H | H | H |
| 955 | General Formula (14) | General Formula (14) | CN | C₂H₅O | General Formula (14) | H | H | H | H | H | H | H | H |
| 956 | General Formula (14) | General Formula (14) | CN | C₂H₅O | General Formula (14) | H | CH₃ | H | H | H | H | H | H |
| 957 | General Formula (14) | General Formula (14) | CN | C₂H₅O | General Formula (14) | H | CH₃O | H | H | H | H | H | H |
| 958 | General Formula (14) | General Formula (14) | CN | C₂H₅O | General Formula (14) | H | t-C₄H₉ | H | H | H | H | H | H |
| 959 | General Formula (14) | General Formula (14) | CN | C₂H₅O | General Formula (14) | H | Cl | H | H | H | H | H | H |
| 560 | General Formula (14) | General Formula (14) | CN | C₂H₅O | General Formula (14) | H | F | H | H | H | H | H | H |
| 961 | General Formula (14) | General Formula (14) | CN | C₆H₅O | General Formula (14) | H | H | H | H | H | H | H | H |
| 962 | General Formula (14) | General Formula (14) | CN | C₆H₅O | General Formula (14) | H | CH₃ | H | H | H | H | H | H |
| 963 | General Formula (14) | General Formula (14) | CN | C₆H₅O | General Formula (14) | H | CH₃O | H | H | H | H | H | H |
| 964 | General Formula (14) | General Formula (14) | CN | C₆H₅O | General Formula (14) | H | t-C₄H₉ | H | H | H | H | H | H |
| 965 | General Formula (14) | General Formula (14) | CN | C₆H₅O | General Formula (14) | H | Cl | H | H | H | H | H | H |
| 966 | General Formula (14) | General Formula (14) | CN | C₆H₅O | General Formula (14) | H | F | H | H | H | H | H | H |
| 967 | General Formula (14) | General Formula (14) | CN | Formula (21) | General Formula (14) | H | H | H | H | H | H | H | H |
| 968 | General Formula (14) | General Formula (14) | CN | Formula (21) | General Formula (14) | H | CH₃ | H | H | H | H | H | H |
| 969 | General Formula (14) | General Formula (14) | CN | Formula (21) | General Formula (14) | H | CH₃O | H | H | H | H | H | H |
| 970 | General Formula (14) | General Formula (14) | CN | Formula (21) | General Formula (14) | H | t-C₄H₉ | H | H | H | H | H | H |
| 971 | General Formula (14) | General Formula (14) | CN | Formula (21) | General Formula (14) | H | Cl | H | H | H | H | H | H |
| 972 | General Formula (14) | General Formula (14) | CN | Formula (21) | General Formula (14) | H | F | H | H | H | H | H | H |
| 973 | General Formula (14) | General Formula (14) | CN | Formula (22) | General Formula (14) | H | H | H | H | H | H | H | H |
| 974 | General Formula (14) | General Formula (14) | CN | Formula (22) | General Formula (14) | H | CH₃ | H | H | H | H | H | H |
| 975 | General Formula (14) | General Formula (14) | CN | Formula (22) | General Formula (14) | H | CH₃O | H | H | H | H | H | H |
| 976 | General Formula (14) | General Formula (14) | CN | Formula (22) | General Formula (14) | H | t-C₄H₉ | H | H | H | H | H | H |
| 977 | General Formula (14) | General Formula (14) | CN | Formula (22) | General Formula (14) | H | Cl | H | H | H | H | H | H |
| 978 | General Formula (14) | General Formula (14) | CN | Formula (22) | General Formula (14) | H | F | H | H | H | H | H | H |
| 989 | General Formula (14) | General Formula (14) | CN | Formula (23) | General Formula (14) | H | H | H | H | H | H | H | H |
| 980 | General Formula (14) | General Formula (14) | CN | Formula (23) | General Formula (14) | H | CH₃ | H | H | H | H | H | H |
| 981 | General Formula (14) | General Formula (14) | CN | Formula (23) | General Formula (14) | H | CH₃O | H | H | H | H | H | H |
| 982 | General Formula (14) | General Formula (14) | CN | Formula (23) | General Formula (14) | H | t-C₄H₉ | H | H | H | H | H | H |
| 983 | General Formula (14) | General Formula (14) | CN | Formula (23) | General Formula (14) | H | Cl | H | H | H | H | H | H |
| 984 | General Formula (14) | General Formula (14) | CN | Formula (23) | General Formula (14) | H | F | H | H | H | H | H | H |
| 985 | General Formula (14) | General Formula (14) | CN | Formula (24) | General Formula (14) | H | H | H | H | H | H | H | H |
| 986 | General Formula (14) | General Formula (14) | CN | Formula (24) | General Formula (14) | H | CH₃ | H | H | H | H | H | H |
| 987 | General Formula (14) | General Formula (14) | CN | Formula (24) | General Formula (14) | H | CH₃O | H | H | H | H | H | H |
| 988 | General Formula (14) | General Formula (14) | CN | Formula (24) | General Formula (14) | H | t-C₄H₉ | H | H | H | H | H | H |
| 989 | General Formula (14) | General Formula (14) | CN | Formula (24) | General Formula (14) | H | Cl | H | H | H | H | H | H |
| 990 | General Formula (14) | General Formula (14) | CN | Formula (24) | General Formula (14) | H | F | H | H | H | H | H | H |

TABLE 7

| Compound | General Formula (1) | | | | | General Formula (15) | | | | |
|---|---|---|---|---|---|---|---|---|---|---|
| No. | R¹ | R² | R³ | R⁴ | R⁵ | R⁷¹, R⁸⁰ | R⁷², R⁷⁹ | R⁷³, R⁷⁸ | R⁷⁴, R⁷⁷ | R⁷⁵, R⁷⁶ |
| 1001 | General Formula (15) | General Formula (15) | CN | General Formula (15) | General Formula (15) | H | H | H | H | H |
| 1002 | General Formula (15) | General Formula (15) | CN | General Formula (15) | General Formula (15) | H | CH₃ | H | H | H |
| 1003 | General Formula (15) | General Formula (15) | CN | General Formula (15) | General Formula (15) | H | CH₃O | H | H | H |
| 1004 | General Formula (15) | General Formula (15) | CN | General Formula (15) | General Formula (15) | H | C₆H₅ | H | H | H |
| 1005 | General Formula (15) | General Formula (15) | CN | General Formula (15) | General Formula (15) | H | CH₃ | H | CH₃ | H |
| 1006 | General Formula (15) | General Formula (15) | CN | General Formula (15) | General Formula (15) | H | CH₃O | H | CH₃O | H |
| 1007 | General Formula (15) | General Formula (15) | CN | General Formula (15) | General Formula (15) | H | C₆H₅ | H | C₆H₅ | H |
| 1008 | General Formula (15) | General Formula (15) | CN | General Formula (15) | General Formula (15) | H | H | CH₃ | H | H |
| 1009 | General Formula (15) | General Formula (15) | CN | General Formula (15) | General Formula (15) | H | H | CH₃O | H | H |
| 1010 | General Formula (15) | General Formula (15) | CN | General Formula (15) | General Formula (15) | H | H | t-C₄H₉ | H | H |
| 1011 | General Formula (15) | General Formula (15) | CN | General Formula (15) | General Formula (15) | H | H | Cl | H | H |
| 1012 | General Formula (15) | General Formula (15) | CN | General Formula (15) | General Formula (15) | H | H | F | H | H |
| 1013 | General Formula (15) | General Formula (15) | CN | General Formula (15) | General Formula (15) | H | H | C₆H₅ | H | H |
| 1014 | General Formula (15) | General Formula (15) | CN | General Formula (15) | General Formula (15) | H | H | p-C₆H₅—C₆H₄ | H | H |
| 1015 | General Formula (15) | General Formula (15) | CN | General Formula (15) | H | H | H | H | H | H |
| 1016 | General Formula (15) | General Formula (15) | CN | H | General Formula (15) | H | H | H | H | H |
| 1017 | General Formula (15) | General Formula (15) | CN | H | H | H | H | H | H | H |
| 1018 | General Formula (15) | H | CN | General Formula (15) | H | H | H | H | H | H |
| 1019 | H | General Formula (15) | CN | General Formula (15) | H | H | H | H | H | H |
| 1020 | General Formula (15) | H | CN | H | H | H | H | H | H | H |
| 1021 | General Formula (15) | General Formula (15) | CN | General Formula (15) | F | H | H | H | H | H |
| 1022 | General Formula (15) | General Formula (15) | CN | F | General Formula (15) | H | H | H | H | H |
| 1023 | General Formula (15) | General Formula (15) | CN | F | F | H | H | H | H | H |
| 1024 | General Formula (15) | F | CN | General Formula (15) | F | H | H | H | H | H |
| 1025 | F | General Formula (15) | CN | General Formula (15) | F | H | H | H | H | H |
| 1026 | General Formula (15) | F | CN | F | F | H | H | H | H | H |
| 1027 | General Formula (15) | General Formula (15) | CN | General Formula (15) | OH | H | H | H | H | H |
| 1028 | General Formula (15) | General Formula (15) | CN | OH | General Formula (15) | H | H | H | H | H |
| 1029 | General Formula (15) | General Formula (15) | CN | OH | OH | H | H | H | H | H |
| 1030 | General Formula (15) | OH | CN | General Formula (15) | OH | H | H | H | H | H |
| 1031 | OH | General Formula (15) | CN | General Formula (15) | OH | H | H | H | H | H |
| 1032 | General Formula (15) | OH | CN | OH | OH | H | H | H | H | H |
| 1033 | General Formula (15) | General Formula (15) | CN | Cl | General Formula (15) | H | H | H | H | H |
| 1034 | General Formula (15) | General Formula (15) | CN | Cl | General Formula (15) | H | H | CH₃ | H | H |
| 1035 | General Formula (15) | General Formula (15) | CN | Cl | General Formula (15) | H | H | CH₃O | H | H |
| 1036 | General Formula (15) | General Formula (15) | CN | Cl | General Formula (15) | H | H | t-C₄H₉ | H | H |
| 1037 | General Formula (15) | General Formula (15) | CN | Cl | General Formula (15) | H | H | Cl | H | H |
| 1038 | General Formula (15) | General Formula (15) | CN | Cl | General Formula (15) | H | H | F | H | H |
| 1039 | General Formula (15) | General Formula (15) | CN | F | General Formula (15) | H | H | H | H | H |
| 1040 | General Formula (15) | General Formula (15) | CN | F | General Formula (15) | H | H | CH₃ | H | H |
| 1041 | General Formula (15) | General Formula (15) | CN | F | General Formula (15) | H | H | CH₃O | H | H |
| 1042 | General Formula (15) | General Formula (15) | CN | F | General Formula (15) | H | H | t-C₄H₉ | H | H |
| 1043 | General Formula (15) | General Formula (15) | CN | F | General Formula (15) | H | H | Cl | H | H |
| 1044 | General Formula (15) | General Formula (15) | CN | F | General Formula (15) | H | H | F | H | H |
| 1045 | General Formula (15) | General Formula (15) | CN | CH₃O | General Formula (15) | H | H | H | H | H |
| 1046 | General Formula (15) | General Formula (15) | CN | CH₃O | General Formula (15) | H | H | CH₃ | H | H |
| 1047 | General Formula (15) | General Formula (15) | CN | CH₃O | General Formula (15) | H | H | CH₃O | H | H |
| 1048 | General Formula (15) | General Formula (15) | CN | CH₃O | General Formula (15) | H | H | t-C₄H₉ | H | H |
| 1049 | General Formula (15) | General Formula (15) | CN | CH₃O | General Formula (15) | H | H | Cl | H | H |
| 1050 | General Formula (15) | General Formula (15) | CN | CH₃O | General Formula (15) | H | H | F | H | H |
| 1051 | General Formula (15) | General Formula (15) | CN | C₂H₅O | General Formula (15) | H | H | H | H | H |
| 1052 | General Formula (15) | General Formula (15) | CN | C₂H₅O | General Formula (15) | H | H | CH₃ | H | H |
| 1053 | General Formula (15) | General Formula (15) | CN | C₂H₅O | General Formula (15) | H | H | CH₃O | H | H |
| 1054 | General Formula (15) | General Formula (15) | CN | C₂H₅O | General Formula (15) | H | H | t-C₄H₉ | H | H |
| 1055 | General Formula (15) | General Formula (15) | CN | C₂H₅O | General Formula (15) | H | H | Cl | H | H |
| 1056 | General Formula (15) | General Formula (15) | CN | C₂H₅O | General Formula (15) | H | H | F | H | H |
| 1057 | General Formula (15) | General Formula (15) | CN | C₆H₅O | General Formula (15) | H | H | H | H | H |
| 1058 | General Formula (15) | General Formula (15) | CN | C₆H₅O | General Formula (15) | H | H | CH₃ | H | H |
| 1059 | General Formula (15) | General Formula (15) | CN | C₆H₅O | General Formula (15) | H | H | CH₃O | H | H |
| 1060 | General Formula (15) | General Formula (15) | CN | C₆H₅O | General Formula (15) | H | H | t-C₄H₉ | H | H |
| 1061 | General Formula (15) | General Formula (15) | CN | C₆H₅O | General Formula (15) | H | H | Cl | H | H |
| 1062 | General Formula (15) | General Formula (15) | CN | C₆H₅O | General Formula (15) | H | H | F | H | H |
| 1063 | General Formula (15) | General Formula (15) | CN | Formula (21) | General Formula (15) | H | H | H | H | H |
| 1064 | General Formula (15) | General Formula (15) | CN | Formula (21) | General Formula (15) | H | H | CH₃ | H | H |
| 1065 | General Formula (15) | General Formula (15) | CN | Formula (21) | General Formula (15) | H | H | CH₃O | H | H |
| 1066 | General Formula (15) | General Formula (15) | CN | Formula (21) | General Formula (15) | H | H | t-C₄H₉ | H | H |
| 1067 | General Formula (15) | General Formula (15) | CN | Formula (21) | General Formula (15) | H | H | Cl | H | H |
| 1068 | General Formula (15) | General Formula (15) | CN | Formula (21) | General Formula (15) | H | H | F | H | H |
| 1069 | General Formula (15) | General Formula (15) | CN | Formula (22) | General Formula (15) | H | H | H | H | H |
| 1070 | General Formula (15) | General Formula (15) | CN | Formula (22) | General Formula (15) | H | H | CH₃ | H | H |
| 1071 | General Formula (15) | General Formula (15) | CN | Formula (22) | General Formula (15) | H | H | CH₃O | H | H |
| 1072 | General Formula (15) | General Formula (15) | CN | Formula (22) | General Formula (15) | H | H | t-C₄H₉ | H | H |
| 1073 | General Formula (15) | General Formula (15) | CN | Formula (22) | General Formula (15) | H | H | Cl | H | H |
| 1074 | General Formula (15) | General Formula (15) | CN | Formula (22) | General Formula (15) | H | H | F | H | H |
| 1075 | General Formula (15) | General Formula (15) | CN | Formula (23) | General Formula (15) | H | H | H | H | H |
| 1076 | General Formula (15) | General Formula (15) | CN | Formula (23) | General Formula (15) | H | H | CH₃ | H | H |
| 1077 | General Formula (15) | General Formula (15) | CN | Formula (23) | General Formula (15) | H | H | CH₃O | H | H |
| 1078 | General Formula (15) | General Formula (15) | CN | Formula (23) | General Formula (15) | H | H | t-C₄H₉ | H | H |
| 1079 | General Formula (15) | General Formula (15) | CN | Formula (23) | General Formula (15) | H | H | Cl | H | H |
| 1080 | General Formula (15) | General Formula (15) | CN | Formula (23) | General Formula (15) | H | H | F | H | H |
| 1081 | General Formula (15) | General Formula (15) | CN | Formula (24) | General Formula (15) | H | H | H | H | H |
| 1082 | General Formula (15) | General Formula (15) | CN | Formula (24) | General Formula (15) | H | H | CH₃ | H | H |
| 1083 | General Formula (15) | General Formula (15) | CN | Formula (24) | General Formula (15) | H | H | CH₃O | H | H |
| 1084 | General Formula (15) | General Formula (15) | CN | Formula (24) | General Formula (15) | H | H | t-C₄H₉ | H | H |
| 1085 | General Formula (15) | General Formula (15) | CN | Formula (24) | General Formula (15) | H | H | Cl | H | H |
| 1086 | General Formula (15) | General Formula (15) | CN | Formula (24) | General Formula (15) | H | H | F | H | H |

TABLE 8

| Compound | General Formula (1) | | | | |
|---|---|---|---|---|---|
| No. | R¹ | R² | R³ | R⁴ | R⁵ |
| 1101 | General Formula (12) | CN | General Formula (12) | General Formula (12) | General Formula (12) |
| 1102 | General Formula (12) | CN | General Formula (12) | General Formula (12) | General Formula (12) |
| 1103 | General Formula (12) | CN | General Formula (12) | General Formula (12) | General Formula (12) |
| 1104 | General Formula (12) | CN | General Formula (12) | General Formula (12) | General Formula (12) |
| 1105 | General Formula (12) | CN | General Formula (12) | General Formula (12) | General Formula (12) |
| 1106 | General Formula (12) | CN | General Formula (12) | General Formula (12) | General Formula (12) |
| 1107 | General Formula (12) | CN | General Formula (12) | General Formula (12) | General Formula (12) |
| 1108 | General Formula (12) | CN | General Formula (12) | General Formula (12) | General Formula (12) |
| 1109 | General Formula (12) | CN | General Formula (12) | General Formula (12) | General Formula (12) |
| 1110 | General Formula (12) | CN | General Formula (12) | General Formula (12) | General Formula (12) |
| 1111 | General Formula (12) | CN | General Formula (12) | General Formula (12) | General Formula (12) |
| 1112 | General Formula (12) | CN | General Formula (12) | General Formula (12) | General Formula (12) |

| Compound | General Formula (12) | | | |
|---|---|---|---|---|
| No. | R³¹, R³⁸ | R³², R³⁷ | R³³, R³⁶ | R³⁴, R³⁵ |
| 1101 | H | H | n-Butyl | H |
| 1102 | H | H | iso-Butyl | H |
| 1103 | H | H | n-Pentyl | H |
| 1104 | H | H | iso-Pentyl | H |
| 1105 | H | H | tert-Pentyl | H |
| 1106 | H | H | neo-Pentyl | H |
| 1107 | H | H | n-Hexyl | H |
| 1108 | H | H | 2-Ethylhexyl | H |
| 1109 | H | H | n-Octyl | H |
| 1110 | H | H | n-Decyl | H |
| 1111 | H | H | n-Undecyl | H |
| 1112 | H | H | n-Dodecyl | H |

Next, compounds represented by the following general formula (B) are described.

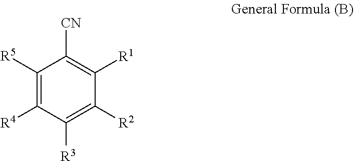

In the general formula (B), one or more of R¹, R², R³, R⁴ and R⁵ each independently represent a 9-carbazolyl group having a substituent at at least one of 1-position and 8-position, a 10-phenoxazyl group having a substituent at at least one of 1-position and 9-position, or a 10-phenothiazyl group having a substituent at at least one of 1-position and 9-position. The remaining substituents each represent a hydrogen atom or a substituent, but the substituent is not a 9-carbazolyl group having a substituent at at least one of 1-position and 8-position, a 10-phenoxazyl group having a substituent at at least one of 1-position and 9-position, or a 10-phenothiazyl group having a substituent at at least one of 1-position and 9-position. One or more carbon atoms constituting each ring skeleton of the 9-carbazolyl group, the 10-phenoxazyl group and the 10-phenothiazyl group may be substituted with a nitrogen atom.

Specific examples (m-D1 to m-D23) of the “9-carbazolyl group having a substituent at at least one of 1-position and 8-position” that one or more of R¹, R², R³, R⁴ and R⁵ represent are shown below.

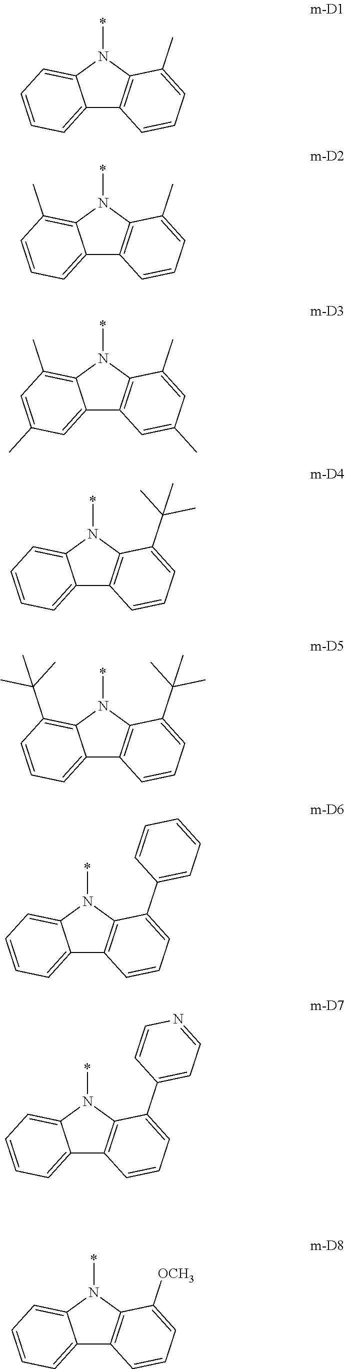

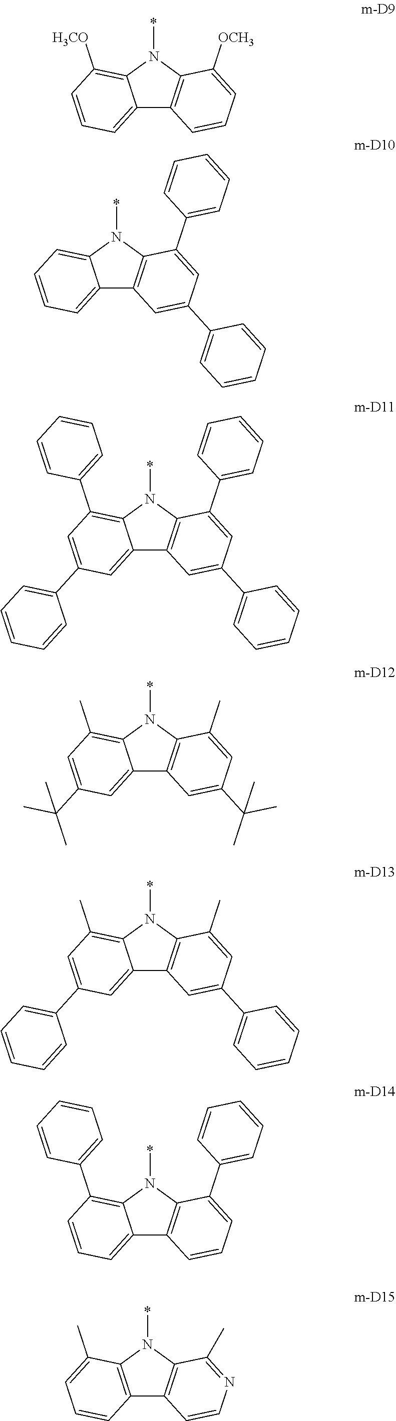

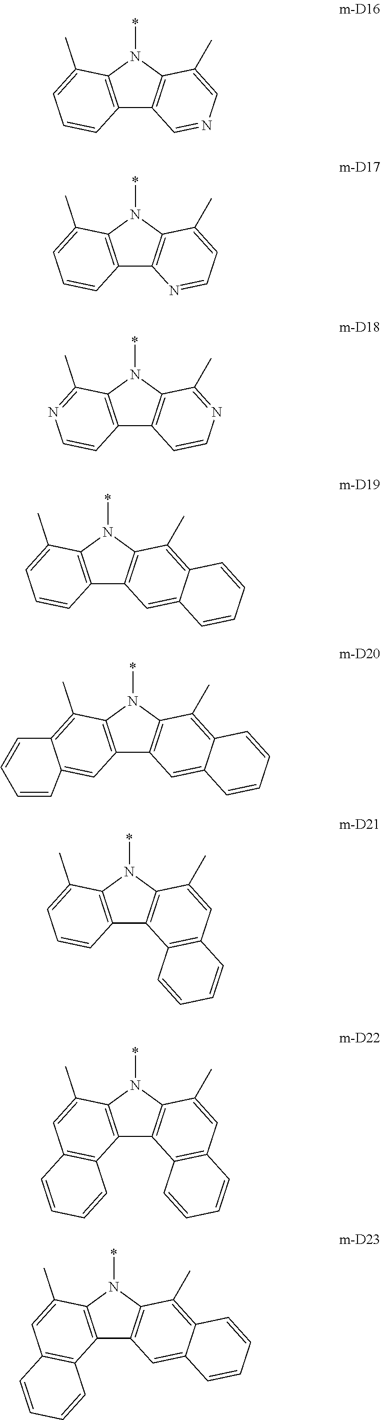

Specific examples (Cz, Cz-1 to Cz-12) of the “substituent” that the other groups than the above-mentioned “one or more” of R¹, R², R³, R⁴ and R⁵ represent are shown below.

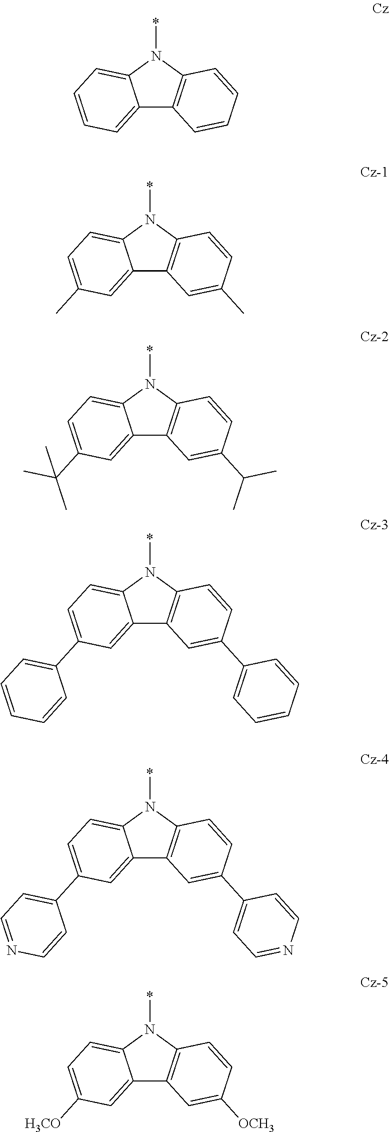

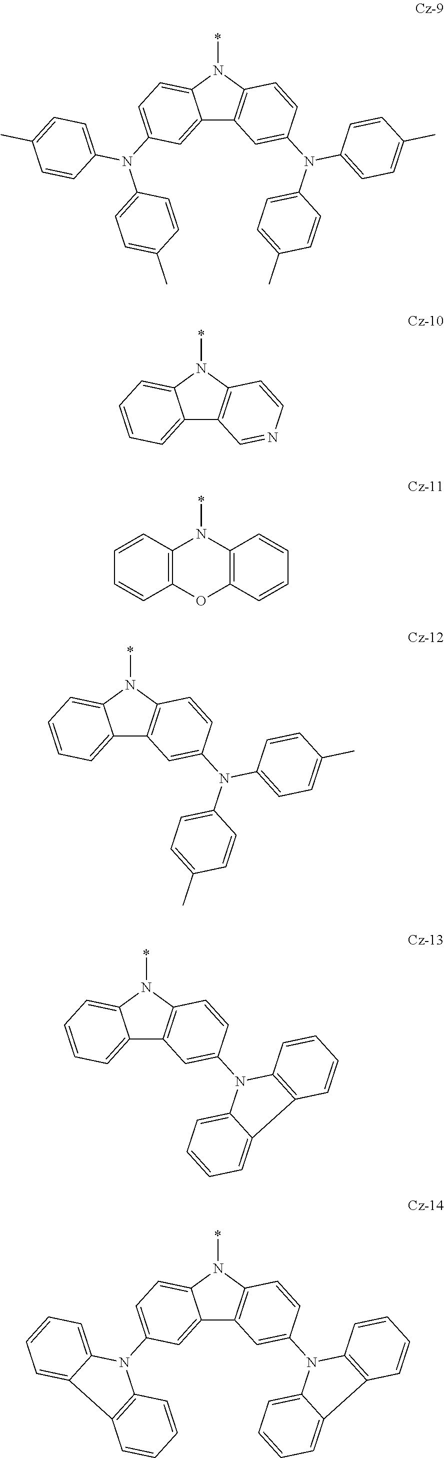

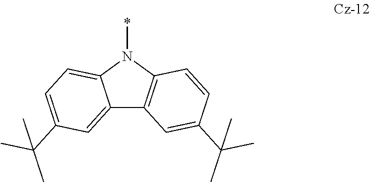 [SMILES: *n1c2ccc(C(C)(C)C)cc2c2cc(C(C)(C)C)ccc21]

Specific examples of the compounds represented by the general formula (B) are shown below.

TABLE 9

| No | R¹ | R² | R³ | R⁴ | R⁵ |
|---|---|---|---|---|---|
| 1 | m-D1 | Cz | Cz | Cz | Cz |
| 2 | Cz | m-D1 | Cz | Cz | Cz |
| 3 | Cz | Cz | m-D1 | Cz | Cz |
| 4 | m-D1 | Cz | Cz | Cz | H |
| 5 | Cz | m-D1 | Cz | Cz | H |
| 6 | Cz | Cz | m-D1 | Cz | H |
| 7 | Cz | Cz | Cz | m-D1 | H |
| 8 | m-D1 | Cz | Cz | H | Cz |
| 9 | Cz | m-D1 | Cz | H | Cz |
| 10 | Cz | Cz | m-D1 | H | Cz |
| 11 | Cz | Cz | Cz | H | m-D1 |
| 12 | m-D1 | Cz | H | Cz | Cz |
| 13 | Cz | m-D1 | H | Cz | Cz |
| 14 | m-D1 | Cz | Cz | H | H |
| 15 | Cz | m-D1 | Cz | H | H |
| 16 | Cz | Cz | m-D1 | H | H |
| 17 | m-D1 | Cz | H | Cz | H |
| 18 | Cz | m-D1 | H | Cz | H |
| 19 | Cz | Cz | H | m-D1 | H |
| 20 | m-D1 | H | Cz | Cz | H |
| 21 | Cz | H | m-D1 | Cz | H |
| 22 | Cz | H | Cz | m-D1 | H |
| 23 | H | m-D1 | Cz | Cz | H |
| 24 | H | Cz | m-D1 | Cz | H |
| 25 | m-D1 | Cz | H | H | Cz |
| 26 | Cz | m-D1 | H | H | Cz |
| 27 | Cz | Cz | H | H | m-D1 |
| 28 | m-D1 | H | Cz | H | Cz |
| 29 | Cz | H | m-D1 | H | Cz |
| 30 | m-D1 | Cz | H | H | H |
| 31 | Cz | m-D1 | H | H | H |
| 32 | m-D1 | H | Cz | H | H |
| 33 | Cz | H | m-D1 | H | H |
| 34 | H | m-D1 | Cz | H | H |
| 35 | H | Cz | m-D1 | H | H |
| 36 | m-D1 | H | H | Cz | H |
| 37 | Cz | H | H | m-D1 | H |
| 38 | H | m-D1 | H | Cz | H |
| 39 | m-D1 | H | H | H | Cz |
| 40 | m-D1 | H | H | H | H |
| 41 | H | m-D1 | H | H | H |
| 42 | H | H | m-D1 | H | H |
| 43 | m-D1 | m-D1 | Cz | Cz | H |
| 44 | m-D1 | Cz | m-D1 | Cz | H |
| 45 | m-D1 | Cz | Cz | m-D1 | H |
| 46 | Cz | m-D1 | m-D1 | Cz | H |
| 47 | Cz | m-D1 | Cz | m-D1 | H |
| 48 | Cz | Cz | m-D1 | m-D1 | H |
| 49 | m-D1 | m-D1 | Cz | H | Cz |
| 50 | m-D1 | Cz | m-D1 | H | Cz |
| 51 | m-D1 | Cz | Cz | H | m-D1 |
| 52 | Cz | m-D1 | m-D1 | H | Cz |
| 53 | Cz | m-D1 | Cz | H | m-D1 |
| 54 | Cz | Cz | m-D1 | H | m-D1 |
| 55 | m-D1 | m-D1 | H | Cz | Cz |
| 56 | m-D1 | Cz | H | m-D1 | Cz |
| 57 | m-D1 | Cz | H | Cz | m-D1 |
| 58 | Cz | m-D1 | H | m-D1 | Cz |
| 59 | m-D1 | m-D1 | Cz | H | H |
| 60 | m-D1 | Cz | m-D1 | H | H |
| 61 | Cz | m-D1 | m-D1 | H | H |
| 62 | m-D1 | m-D1 | H | Cz | H |
| 63 | m-D1 | Cz | H | m-D1 | H |
| 64 | Cz | m-D1 | H | m-D1 | H |
| 65 | m-D1 | H | m-D1 | Cz | H |
| 66 | m-D1 | H | Cz | m-D1 | H |
| 67 | Cz | H | m-D1 | m-D1 | H |
| 68 | H | m-D1 | m-D1 | Cz | H |
| 69 | H | m-D1 | Cz | m-D1 | H |
| 70 | m-D1 | m-D1 | H | H | Cz |
| 71 | m-D1 | Cz | H | H | m-D1 |
| 72 | Cz | m-D1 | H | H | m-D1 |
| 73 | m-D1 | H | m-D1 | H | Cz |
| 74 | m-D1 | H | Cz | H | m-D1 |
| 75 | m-D1 | m-D1 | H | H | H |
| 76 | m-D1 | H | m-D1 | H | H |
| 77 | H | m-D1 | m-D1 | H | H |
| 78 | m-D1 | H | H | m-D1 | H |
| 79 | H | m-D1 | H | m-D1 | H |
| 80 | m-D1 | H | H | H | m-D1 |
| 81 | m-D1 | m-D1 | m-D1 | Cz | Cz |
| 82 | m-D1 | m-D1 | Cz | m-D1 | Cz |
| 83 | m-D1 | m-D1 | Cz | Cz | m-D1 |
| 84 | m-D1 | Cz | m-D1 | m-D1 | Cz |
| 85 | m-D1 | Cz | m-D1 | Cz | m-D1 |
| 86 | m-D1 | m-D1 | m-D1 | Cz | H |
| 87 | m-D1 | m-D1 | Cz | m-D1 | H |
| 88 | m-D1 | Cz | m-D1 | Cz | H |
| 89 | Cz | m-D1 | m-D1 | m-D1 | H |
| 90 | m-D1 | m-D1 | m-D1 | H | Cz |
| 91 | m-D1 | m-D1 | Cz | H | m-D1 |
| 92 | m-D1 | Cz | m-D1 | H | m-D1 |
| 93 | Cz | m-D1 | m-D1 | H | m-D1 |
| 94 | m-D1 | m-D1 | H | m-D1 | Cz |
| 95 | m-D1 | m-D1 | H | Cz | m-D1 |
| 96 | m-D1 | m-D1 | m-D1 | H | H |
| 97 | m-D1 | m-D1 | H | m-D1 | H |
| 98 | m-D1 | H | m-D1 | m-D1 | H |
| 99 | H | m-D1 | m-D1 | m-D1 | H |
| 100 | m-D1 | m-D1 | m-D1 | m-D1 | Cz |
| 101 | m-D1 | m-D1 | m-D1 | Cz | m-D1 |
| 102 | m-D1 | m-D1 | Cz | m-D1 | m-D1 |
| 103 | m-D1 | m-D1 | m-D1 | m-D1 | H |
| 104 | m-D1 | m-D1 | m-D1 | H | m-D1 |
| 105 | m-D1 | m-D1 | H | m-D1 | m-D1 |
| 106 | m-D1 | m-D1 | m-D1 | m-D1 | m-D1 |
| 107 | m-D2 | Cz | Cz | Cz | Cz |
| 108 | Cz | m-D2 | Cz | Cz | Cz |
| 109 | Cz | Cz | m-D2 | Cz | Cz |
| 110 | m-D2 | Cz | Cz | Cz | H |
| 111 | Cz | m-D2 | Cz | Cz | H |
| 112 | Cz | Cz | m-D2 | Cz | H |
| 113 | Cz | Cz | Cz | m-D2 | H |
| 114 | m-D2 | Cz | Cz | H | Cz |
| 115 | Cz | m-D2 | Cz | H | Cz |
| 116 | Cz | Cz | m-D2 | H | Cz |
| 117 | Cz | Cz | Cz | H | m-D2 |
| 118 | m-D2 | Cz | H | Cz | Cz |
| 119 | Cz | m-D2 | H | Cz | Cz |
| 120 | m-D2 | Cz | Cz | H | H |
| 121 | Cz | m-D2 | Cz | H | H |
| 122 | Cz | Cz | m-D2 | H | H |
| 123 | m-D2 | Cz | H | Cz | H |
| 124 | Cz | m-D2 | H | Cz | H |
| 125 | Cz | Cz | H | m-D2 | H |
| 126 | m-D2 | H | Cz | Cz | H |
| 127 | Cz | H | m-D2 | Cz | H |
| 128 | Cz | H | Cz | m-D2 | H |
| 129 | H | m-D2 | Cz | Cz | H |
| 130 | H | Cz | m-D2 | Cz | H |
| 131 | m-D2 | Cz | H | H | Cz |
| 132 | Cz | m-D2 | H | H | Cz |
| 133 | Cz | Cz | H | H | m-D2 |
| 134 | m-D2 | H | Cz | H | Cz |
| 135 | Cz | H | m-D2 | H | Cz |
| 136 | m-D2 | Cz | H | H | H |
| 137 | Cz | m-D2 | H | H | H |
| 138 | m-D2 | H | Cz | H | H |
| 139 | Cz | H | m-D2 | H | H |
| 140 | H | m-D2 | Cz | H | H |
| 141 | H | Cz | m-D2 | H | H |
| 142 | m-D2 | H | H | Cz | H |
| 143 | Cz | H | H | m-D2 | H |
| 144 | H | m-D2 | H | Cz | H |
| 145 | m-D2 | H | H | H | Cz |
| 146 | m-D2 | H | H | H | H |
| 147 | H | m-D2 | H | H | H |
| 148 | H | H | m-D2 | H | H |
| 149 | m-D2 | m-D2 | Cz | Cz | H |
| 150 | m-D2 | Cz | m-D2 | Cz | H |
| 151 | m-D2 | Cz | Cz | m-D2 | H |
| 152 | Cz | m-D2 | m-D2 | Cz | H |
| 153 | Cz | m-D2 | Cz | m-D2 | H |
| 154 | Cz | Cz | m-D2 | m-D2 | H |
| 155 | m-D2 | m-D2 | Cz | H | Cz |
| 156 | m-D2 | Cz | m-D2 | H | Cz |
| 157 | m-D2 | Cz | Cz | H | m-D2 |
| 158 | Cz | m-D2 | m-D2 | H | Cz |
| 159 | Cz | m-D2 | Cz | H | m-D2 |
| 160 | Cz | Cz | m-D2 | H | m-D2 |
| 161 | m-D2 | m-D2 | H | Cz | Cz |
| 162 | m-D2 | Cz | H | m-D2 | Cz |
| 163 | m-D2 | Cz | H | Cz | m-D2 |
| 164 | Cz | m-D2 | H | m-D2 | Cz |
| 165 | m-D2 | m-D2 | Cz | H | H |
| 166 | m-D2 | Cz | m-D2 | H | H |
| 167 | Cz | m-D2 | m-D2 | H | H |
| 168 | m-D2 | m-D2 | H | Cz | H |
| 169 | m-D2 | Cz | H | m-D2 | H |
| 170 | Cz | m-D2 | H | m-D2 | H |
| 171 | m-D2 | H | m-D2 | Cz | H |
| 172 | m-D2 | H | Cz | m-D2 | H |
| 173 | Cz | H | m-D2 | m-D2 | H |
| 174 | H | m-D2 | m-D2 | Cz | H |
| 175 | H | m-D2 | Cz | m-D2 | H |
| 176 | m-D2 | m-D2 | H | H | Cz |
| 177 | m-D2 | Cz | H | H | m-D2 |
| 178 | Cz | m-D2 | H | H | m-D2 |
| 179 | m-D2 | H | m-D2 | H | Cz |
| 180 | m-D2 | H | Cz | H | m-D2 |
| 181 | m-D2 | m-D2 | H | H | H |
| 182 | m-D2 | H | m-D2 | H | H |
| 183 | H | m-D2 | m-D2 | H | H |
| 184 | m-D2 | H | H | m-D2 | H |
| 185 | H | m-D2 | H | m-D2 | H |
| 186 | m-D2 | H | H | H | m-D2 |
| 187 | m-D2 | m-D2 | m-D2 | Cz | Cz |
| 188 | m-D2 | m-D2 | Cz | m-D2 | Cz |
| 189 | m-D2 | m-D2 | Cz | Cz | m-D2 |
| 190 | m-D2 | Cz | m-D2 | m-D2 | Cz |
| 191 | m-D2 | Cz | m-D2 | Cz | m-D2 |
| 192 | m-D2 | m-D2 | m-D2 | Cz | H |
| 193 | m-D2 | m-D2 | Cz | m-D2 | H |
| 194 | m-D2 | Cz | m-D2 | Cz | H |
| 195 | Cz | m-D2 | m-D2 | m-D2 | H |
| 196 | m-D2 | m-D2 | m-D2 | H | Cz |
| 197 | m-D2 | m-D2 | Cz | H | m-D2 |
| 198 | m-D2 | Cz | m-D2 | H | m-D2 |
| 199 | Cz | m-D2 | m-D2 | H | m-D2 |
| 200 | m-D2 | m-D2 | H | m-D2 | Cz |
| 201 | m-D2 | m-D2 | H | Cz | m-D2 |
| 202 | m-D2 | m-D2 | m-D2 | H | H |
| 203 | m-D2 | m-D2 | H | m-D2 | H |
| 204 | m-D2 | H | m-D2 | m-D2 | H |
| 205 | H | m-D2 | m-D2 | m-D2 | H |
| 206 | m-D2 | m-D2 | m-D2 | m-D2 | Cz |
| 207 | m-D2 | m-D2 | m-D2 | Cz | m-D2 |
| 208 | m-D2 | m-D2 | Cz | m-D2 | m-D2 |
| 209 | m-D2 | m-D2 | m-D2 | m-D2 | H |
| 210 | m-D2 | m-D2 | m-D2 | H | m-D2 |
| 211 | m-D2 | m-D2 | H | m-D2 | m-D2 |
| 212 | m-D2 | m-D2 | m-D2 | m-D2 | m-D2 |
| 213 | m-D3 | Cz | Cz | Cz | Cz |
| 214 | Cz | m-D3 | Cz | Cz | Cz |
| 215 | Cz | Cz | m-D3 | Cz | Cz |
| 216 | m-D3 | Cz | Cz | Cz | H |
| 217 | Cz | m-D3 | Cz | Cz | H |
| 218 | Cz | Cz | m-D3 | Cz | H |
| 219 | Cz | Cz | Cz | m-D3 | H |
| 220 | m-D3 | Cz | Cz | H | Cz |
| 221 | Cz | m-D3 | Cz | H | Cz |
| 222 | Cz | Cz | m-D3 | H | Cz |
| 223 | Cz | Cz | Cz | H | m-D3 |
| 224 | m-D3 | Cz | H | Cz | Cz |
| 225 | Cz | m-D3 | H | Cz | Cz |
| 226 | m-D3 | Cz | Cz | H | H |
| 227 | Cz | m-D3 | Cz | H | H |
| 228 | Cz | Cz | m-D3 | H | H |
| 229 | m-D3 | Cz | H | Cz | H |
| 230 | Cz | m-D3 | H | Cz | H |
| 231 | Cz | Cz | H | m-D3 | H |
| 232 | m-D3 | H | Cz | Cz | H |
| 233 | Cz | H | m-D3 | Cz | H |
| 234 | Cz | H | Cz | m-D3 | H |
| 235 | H | m-D3 | Cz | Cz | H |
| 236 | H | Cz | m-D3 | Cz | H |
| 237 | m-D3 | Cz | H | H | Cz |
| 238 | Cz | m-D3 | H | H | Cz |
| 239 | Cz | Cz | H | H | m-D3 |
| 240 | m-D3 | H | Cz | H | Cz |
| 241 | Cz | H | m-D3 | H | Cz |
| 242 | m-D3 | Cz | H | H | H |
| 243 | Cz | m-D3 | H | H | H |
| 244 | m-D3 | H | Cz | H | H |
| 245 | Cz | H | m-D3 | H | H |
| 246 | H | m-D3 | Cz | H | H |
| 247 | H | Cz | m-D3 | H | H |
| 248 | m-D3 | H | H | Cz | H |
| 249 | Cz | H | H | m-D3 | H |
| 250 | H | m-D3 | H | Cz | H |
| 251 | m-D3 | H | H | H | Cz |
| 252 | m-D3 | H | H | H | H |
| 253 | H | m-D3 | H | H | H |
| 254 | H | H | m-D3 | H | H |
| 255 | m-D3 | m-D3 | Cz | Cz | H |
| 256 | m-D3 | Cz | m-D3 | Cz | H |
| 257 | m-D3 | Cz | Cz | m-D3 | H |
| 258 | Cz | m-D3 | m-D3 | Cz | H |
| 259 | Cz | m-D3 | Cz | m-D3 | H |
| 260 | Cz | Cz | m-D3 | m-D3 | H |
| 261 | m-D3 | m-D3 | Cz | H | Cz |
| 262 | m-D3 | Cz | m-D3 | H | Cz |
| 263 | m-D3 | Cz | Cz | H | m-D3 |
| 264 | Cz | m-D3 | m-D3 | H | Cz |
| 265 | Cz | m-D3 | Cz | H | m-D3 |
| 266 | Cz | Cz | m-D3 | H | m-D3 |
| 267 | m-D3 | m-D3 | H | Cz | Cz |
| 268 | m-D3 | Cz | H | m-D3 | Cz |
| 269 | m-D3 | Cz | H | Cz | m-D3 |
| 270 | Cz | m-D3 | H | m-D3 | Cz |
| 271 | m-D3 | m-D3 | Cz | H | H |
| 272 | m-D3 | Cz | m-D3 | H | H |
| 273 | Cz | m-D3 | m-D3 | H | H |
| 274 | m-D3 | m-D3 | H | Cz | H |
| 275 | m-D3 | Cz | H | m-D3 | H |
| 276 | Cz | m-D3 | H | m-D3 | H |
| 277 | m-D3 | H | m-D3 | Cz | H |
| 278 | m-D3 | H | Cz | m-D3 | H |
| 279 | Cz | H | m-D3 | m-D3 | H |
| 280 | H | m-D3 | m-D3 | Cz | H |
| 281 | H | m-D3 | Cz | m-D3 | H |
| 282 | m-D3 | m-D3 | H | H | Cz |
| 283 | m-D3 | Cz | H | H | m-D3 |
| 284 | Cz | m-D3 | H | H | m-D3 |
| 285 | m-D3 | H | m-D3 | H | Cz |
| 286 | m-D3 | H | Cz | H | m-D3 |
| 287 | m-D3 | m-D3 | H | H | H |
| 288 | m-D3 | H | m-D3 | H | H |
| 289 | H | m-D3 | m-D3 | H | H |
| 290 | m-D3 | H | H | m-D3 | H |
| 291 | H | m-D3 | H | m-D3 | H |
| 292 | m-D3 | H | H | H | m-D3 |
| 293 | m-D3 | m-D3 | m-D3 | Cz | Cz |
| 294 | m-D3 | m-D3 | Cz | m-D3 | Cz |
| 295 | m-D3 | m-D3 | Cz | Cz | m-D3 |
| 296 | m-D3 | Cz | m-D3 | m-D3 | Cz |
| 297 | m-D3 | Cz | m-D3 | Cz | m-D3 |
| 298 | m-D3 | m-D3 | m-D3 | Cz | H |
| 299 | m-D3 | m-D3 | Cz | m-D3 | H |
| 300 | m-D3 | Cz | m-D3 | Cz | H |
| 301 | Cz | m-D3 | m-D3 | m-D3 | H |
| 302 | m-D3 | m-D3 | m-D3 | H | Cz |
| 303 | m-D3 | m-D3 | Cz | H | m-D3 |
| 304 | m-D3 | Cz | m-D3 | H | m-D3 |
| 305 | Cz | m-D3 | m-D3 | H | m-D3 |
| 306 | m-D3 | m-D3 | H | m-D3 | Cz |
| 307 | m-D3 | m-D3 | H | Cz | m-D3 |
| 308 | m-D3 | m-D3 | m-D3 | H | H |
| 309 | m-D3 | m-D3 | H | m-D3 | H |
| 310 | m-D3 | H | m-D3 | m-D3 | H |
| 311 | H | m-D3 | m-D3 | m-D3 | H |
| 312 | m-D3 | m-D3 | m-D3 | m-D3 | Cz |
| 313 | m-D3 | m-D3 | m-D3 | Cz | m-D3 |
| 314 | m-D3 | m-D3 | Cz | m-D3 | m-D3 |
| 315 | m-D3 | m-D3 | m-D3 | m-D3 | H |
| 316 | m-D3 | m-D3 | m-D3 | H | m-D3 |
| 317 | m-D3 | m-D3 | H | m-D3 | m-D3 |
| 318 | m-D3 | m-D3 | m-D3 | m-D3 | m-D3 |
| 319 | Cz | Cz | m-D4 | Cz | Cz |
| 320 | m-D4 | Cz | Cz | Cz | H |
| 321 | Cz | Cz | Cz | m-D4 | H |
| 322 | m-D4 | Cz | Cz | H | Cz |
| 323 | Cz | Cz | m-D4 | H | Cz |
| 324 | Cz | m-D4 | H | Cz | Cz |
| 325 | Cz | m-D4 | Cz | H | H |
| 326 | Cz | Cz | m-D4 | H | H |
| 327 | H | m-D4 | Cz | Cz | H |
| 328 | H | Cz | m-D4 | Cz | H |
| 329 | m-D4 | H | Cz | H | Cz |
| 330 | Cz | H | m-D4 | H | Cz |
| 331 | m-D4 | Cz | H | H | H |
| 332 | H | m-D4 | H | Cz | H |
| 333 | m-D4 | m-D4 | Cz | H | Cz |
| 334 | m-D4 | Cz | m-D4 | H | Cz |
| 335 | Cz | m-D4 | m-D4 | H | Cz |
| 336 | m-D4 | m-D4 | H | Cz | Cz |
| 337 | Cz | m-D4 | H | m-D4 | Cz |
| 338 | m-D4 | Cz | m-D4 | H | H |
| 339 | H | m-D4 | m-D4 | Cz | H |
| 340 | H | m-D4 | Cz | m-D4 | H |
| 341 | m-D4 | H | m-D4 | H | Cz |
| 342 | m-D4 | H | Cz | H | m-D4 |
| 343 | H | m-D4 | m-D4 | H | H |
| 344 | m-D4 | Cz | m-D4 | Cz | m-D4 |
| 345 | m-D4 | m-D4 | m-D4 | Cz | H |
| 346 | m-D4 | m-D4 | Cz | m-D4 | H |
| 347 | m-D4 | m-D4 | m-D4 | H | Cz |
| 348 | m-D4 | m-D4 | H | m-D4 | Cz |
| 349 | m-D4 | m-D4 | H | Cz | m-D4 |
| 350 | m-D4 | m-D4 | H | m-D4 | H |
| 351 | H | m-D4 | m-D4 | m-D4 | H |
| 352 | m-D4 | m-D4 | m-D4 | m-D4 | Cz |
| 353 | m-D4 | m-D4 | m-D4 | m-D4 | H |
| 354 | m-D4 | m-D4 | H | m-D4 | m-D4 |
| 355 | m-D4 | m-D4 | m-D4 | m-D4 | m-D4 |
| 356 | Cz | Cz | m-D5 | Cz | Cz |
| 357 | m-D5 | Cz | Cz | Cz | H |
| 358 | Cz | Cz | Cz | m-D5 | H |
| 359 | m-D5 | Cz | Cz | H | Cz |
| 360 | Cz | Cz | m-D5 | H | Cz |
| 361 | Cz | m-D5 | H | Cz | Cz |
| 362 | Cz | m-D5 | Cz | H | H |
| 363 | Cz | Cz | m-D5 | H | H |
| 364 | H | m-D5 | Cz | Cz | H |
| 365 | H | Cz | m-D5 | Cz | H |
| 366 | m-D5 | H | Cz | H | Cz |
| 367 | Cz | H | m-D5 | H | Cz |
| 368 | m-D5 | Cz | H | H | H |
| 369 | H | m-D5 | H | Cz | H |
| 370 | m-D5 | m-D5 | Cz | H | Cz |
| 371 | m-D5 | Cz | m-D5 | H | Cz |
| 372 | Cz | m-D5 | m-D5 | H | Cz |
| 373 | m-D5 | m-D5 | H | Cz | Cz |
| 374 | Cz | m-D5 | H | m-D5 | Cz |
| 375 | m-D5 | Cz | m-D5 | H | H |
| 376 | H | m-D5 | m-D5 | Cz | H |
| 377 | H | m-D5 | Cz | m-D5 | H |
| 378 | m-D5 | H | m-D5 | H | Cz |
| 379 | m-D5 | H | Cz | H | m-D5 |
| 380 | H | m-D5 | m-D5 | H | H |
| 381 | m-D5 | Cz | m-D5 | Cz | m-D5 |
| 382 | m-D5 | m-D5 | m-D5 | Cz | H |
| 383 | m-D5 | m-D5 | Cz | m-D5 | H |
| 384 | m-D5 | m-D5 | m-D5 | H | Cz |
| 385 | m-D5 | m-D5 | H | m-D5 | Cz |
| 386 | m-D5 | m-D5 | H | Cz | m-D5 |
| 387 | m-D5 | m-D5 | H | m-D5 | H |
| 388 | H | m-D5 | m-D5 | m-D5 | H |
| 389 | m-D5 | m-D5 | m-D5 | m-D5 | Cz |
| 390 | m-D5 | m-D5 | m-D5 | m-D5 | H |
| 391 | m-D5 | m-D5 | H | m-D5 | m-D5 |
| 392 | m-D5 | m-D5 | m-D5 | m-D5 | m-D5 |
| 393 | Cz | Cz | m-D6 | Cz | Cz |
| 394 | m-D6 | Cz | Cz | Cz | H |
| 395 | Cz | Cz | Cz | m-D6 | H |
| 396 | m-D6 | Cz | Cz | H | Cz |
| 397 | Cz | Cz | m-D6 | H | Cz |
| 398 | Cz | m-D6 | H | Cz | Cz |
| 399 | Cz | m-D6 | Cz | H | H |
| 400 | Cz | Cz | m-D6 | H | H |
| 401 | H | m-D6 | Cz | Cz | H |
| 402 | H | Cz | m-D6 | Cz | H |
| 403 | m-D6 | H | Cz | H | Cz |
| 404 | Cz | H | m-D6 | H | Cz |
| 405 | m-D6 | Cz | H | H | H |
| 406 | H | m-D6 | H | Cz | H |
| 407 | m-D6 | m-D6 | Cz | H | Cz |
| 408 | m-D6 | Cz | m-D6 | H | Cz |
| 409 | Cz | m-D6 | m-D6 | H | Cz |
| 410 | m-D6 | m-D6 | H | Cz | Cz |
| 411 | Cz | m-D6 | H | m-D6 | Cz |
| 412 | m-D6 | Cz | m-D6 | H | H |
| 413 | H | m-D6 | m-D6 | Cz | H |
| 414 | H | m-D6 | Cz | m-D6 | H |
| 415 | m-D6 | H | m-D6 | H | Cz |
| 416 | m-D6 | H | Cz | H | m-D6 |
| 417 | H | m-D6 | m-D6 | H | H |
| 418 | m-D6 | Cz | m-D6 | Cz | m-D6 |
| 419 | m-D6 | m-D6 | m-D6 | Cz | H |
| 420 | m-D6 | m-D6 | Cz | m-D6 | H |
| 421 | m-D6 | m-D6 | m-D6 | H | Cz |
| 422 | m-D6 | m-D6 | H | m-D6 | Cz |
| 423 | m-D6 | m-D6 | H | Cz | m-D6 |
| 424 | m-D6 | m-D6 | H | m-D6 | H |
| 425 | H | m-D6 | m-D6 | m-D6 | H |
| 426 | m-D6 | m-D6 | m-D6 | m-D6 | Cz |
| 427 | m-D6 | m-D6 | m-D6 | m-D6 | H |
| 428 | m-D6 | m-D6 | H | m-D6 | m-D6 |
| 429 | m-D6 | m-D6 | m-D6 | m-D6 | m-D6 |
| 430 | Cz | Cz | m-D7 | Cz | Cz |
| 431 | m-D7 | Cz | Cz | Cz | H |
| 432 | Cz | Cz | Cz | m-D7 | H |
| 433 | m-D7 | Cz | Cz | H | Cz |
| 434 | Cz | Cz | m-D7 | H | Cz |
| 435 | Cz | m-D7 | H | Cz | Cz |
| 436 | Cz | m-D7 | Cz | H | H |
| 437 | Cz | Cz | m-D7 | H | H |
| 438 | H | m-D7 | Cz | Cz | H |
| 439 | H | Cz | m-D7 | Cz | H |
| 440 | m-D7 | H | Cz | H | Cz |
| 441 | Cz | H | m-D7 | H | Cz |
| 442 | m-D7 | Cz | H | H | H |
| 443 | H | m-D7 | H | Cz | H |
| 444 | m-D7 | m-D7 | Cz | H | Cz |
| 445 | m-D7 | Cz | m-D7 | H | Cz |
| 446 | Cz | m-D7 | m-D7 | H | Cz |
| 447 | m-D7 | m-D7 | H | Cz | Cz |
| 448 | Cz | m-D7 | H | m-D7 | Cz |
| 449 | m-D7 | Cz | m-D7 | H | H |
| 450 | H | m-D7 | m-D7 | Cz | H |
| 451 | H | m-D7 | Cz | m-D7 | H |
| 452 | m-D7 | H | m-D7 | H | Cz |
| 453 | m-D7 | H | Cz | H | m-D7 |
| 454 | H | m-D7 | m-D7 | H | H |
| 455 | m-D7 | Cz | m-D7 | Cz | m-D7 |
| 456 | m-D7 | m-D7 | m-D7 | Cz | H |
| 457 | m-D7 | m-D7 | Cz | m-D7 | H |
| 458 | m-D7 | m-D7 | m-D7 | H | Cz |
| 459 | m-D7 | m-D7 | H | m-D7 | Cz |
| 460 | m-D7 | m-D7 | H | Cz | m-D7 |
| 461 | m-D7 | m-D7 | H | m-D7 | H |
| 462 | H | m-D7 | m-D7 | m-D7 | H |
| 463 | m-D7 | m-D7 | m-D7 | m-D7 | Cz |
| 464 | m-D7 | m-D7 | m-D7 | m-D7 | H |
| 465 | m-D7 | m-D7 | H | m-D7 | m-D7 |
| 466 | m-D7 | m-D7 | m-D7 | m-D7 | m-D7 |
| 467 | Cz | Cz | m-D8 | Cz | Cz |
| 468 | m-D8 | Cz | Cz | Cz | H |
| 469 | Cz | Cz | Cz | m-D8 | H |
| 470 | m-D8 | Cz | Cz | H | Cz |
| 471 | Cz | Cz | m-D8 | H | Cz |
| 472 | Cz | m-D8 | H | Cz | Cz |
| 473 | Cz | m-D8 | Cz | H | H |
| 474 | Cz | Cz | m-D8 | H | H |
| 475 | H | m-D8 | Cz | Cz | H |
| 476 | H | Cz | m-D8 | Cz | H |
| 477 | m-D8 | H | Cz | H | Cz |
| 478 | Cz | H | m-D8 | H | Cz |
| 479 | m-D8 | Cz | H | H | H |
| 480 | H | m-D8 | H | Cz | H |
| 481 | m-D8 | m-D8 | Cz | H | Cz |
| 482 | m-D8 | Cz | m-D8 | H | Cz |
| 483 | Cz | m-D8 | m-D8 | H | Cz |
| 484 | m-D8 | m-D8 | H | Cz | Cz |
| 485 | Cz | m-D8 | H | m-D8 | Cz |
| 486 | m-D8 | Cz | m-D8 | H | H |
| 487 | H | m-D8 | m-D8 | Cz | H |
| 488 | H | m-D8 | Cz | m-D8 | H |
| 489 | m-D8 | H | m-D8 | H | Cz |
| 490 | m-D8 | H | Cz | H | m-D8 |
| 491 | H | m-D8 | m-D8 | H | H |
| 492 | m-D8 | Cz | m-D8 | Cz | m-D8 |
| 493 | m-D8 | m-D8 | m-D8 | Cz | H |
| 494 | m-D8 | m-D8 | Cz | m-D8 | H |
| 495 | m-D8 | m-D8 | m-D8 | H | Cz |
| 496 | m-D8 | m-D8 | H | m-D8 | Cz |
| 497 | m-D8 | m-D8 | H | Cz | m-D8 |
| 498 | m-D8 | m-D8 | H | m-D8 | H |
| 499 | H | m-D8 | m-D8 | m-D8 | H |
| 500 | m-D8 | m-D8 | m-D8 | m-D8 | Cz |
| 501 | m-D8 | m-D8 | m-D8 | m-D8 | H |
| 502 | m-D8 | m-D8 | H | m-D8 | m-D8 |
| 503 | m-D8 | m-D8 | m-D8 | m-D8 | m-D8 |
| 504 | Cz | Cz | m-D9 | Cz | Cz |
| 505 | m-D9 | Cz | Cz | Cz | H |
| 506 | Cz | Cz | Cz | m-D9 | H |
| 507 | m-D9 | Cz | Cz | H | Cz |
| 508 | Cz | Cz | m-D9 | H | Cz |
| 509 | Cz | m-D9 | H | Cz | Cz |
| 510 | Cz | m-D9 | Cz | H | H |
| 511 | Cz | Cz | m-D9 | H | H |
| 512 | H | m-D9 | Cz | Cz | H |
| 513 | H | Cz | m-D9 | Cz | H |
| 514 | m-D9 | H | Cz | H | Cz |
| 515 | Cz | H | m-D9 | H | Cz |
| 516 | m-D9 | Cz | H | H | H |
| 517 | H | m-D9 | H | Cz | H |
| 518 | m-D9 | m-D9 | Cz | H | Cz |
| 519 | m-D9 | Cz | m-D9 | H | Cz |
| 520 | Cz | m-D9 | m-D9 | H | Cz |
| 521 | m-D9 | m-D9 | H | Cz | Cz |
| 522 | Cz | m-D9 | H | m-D9 | Cz |
| 523 | m-D9 | Cz | m-D9 | H | H |
| 524 | H | m-D9 | m-D9 | Cz | H |
| 525 | H | m-D9 | Cz | m-D9 | H |
| 526 | m-D9 | H | m-D9 | H | Cz |
| 527 | m-D9 | H | Cz | H | m-D9 |
| 528 | H | m-D9 | m-D9 | H | H |
| 529 | m-D9 | Cz | m-D9 | Cz | m-D9 |
| 530 | m-D9 | m-D9 | m-D9 | Cz | H |
| 531 | m-D9 | m-D9 | Cz | m-D9 | H |
| 532 | m-D9 | m-D9 | m-D9 | H | Cz |
| 533 | m-D9 | m-D9 | H | m-D9 | Cz |
| 534 | m-D9 | m-D9 | H | Cz | m-D9 |
| 535 | m-D9 | m-D9 | H | m-D9 | H |
| 536 | H | m-D9 | m-D9 | m-D9 | H |
| 537 | m-D9 | m-D9 | m-D9 | m-D9 | Cz |
| 538 | m-D9 | m-D9 | m-D9 | m-D9 | H |
| 539 | m-D9 | m-D9 | H | m-D9 | m-D9 |
| 540 | m-D9 | m-D9 | m-D9 | m-D9 | m-D9 |
| 541 | m-D1 | Cz1 | Cz1 | Cz1 | Cz1 |
| 542 | Cz1 | m-D1 | Cz1 | Cz1 | Cz1 |
| 543 | Cz1 | Cz1 | m-D1 | Cz1 | Cz1 |
| 544 | m-D1 | Cz1 | Cz1 | Cz1 | H |
| 545 | Cz1 | m-D1 | Cz1 | Cz1 | H |
| 546 | Cz1 | Cz1 | m-D1 | Cz1 | H |
| 547 | Cz1 | Cz1 | Cz1 | m-D1 | H |
| 548 | m-D1 | Cz1 | Cz1 | H | Cz1 |
| 549 | Cz1 | m-D1 | Cz1 | H | Cz1 |
| 550 | Cz1 | Cz1 | m-D1 | H | Cz1 |
| 551 | Cz1 | Cz1 | Cz1 | H | m-D1 |
| 552 | m-D1 | Cz1 | H | Cz1 | Cz1 |
| 553 | Cz1 | m-D1 | H | Cz1 | Cz1 |
| 554 | m-D1 | Cz1 | Cz1 | H | H |
| 555 | Cz1 | m-D1 | Cz1 | H | H |
| 556 | Cz1 | Cz1 | m-D1 | H | H |
| 557 | m-D1 | Cz1 | H | Cz1 | H |
| 558 | Cz1 | m-D1 | H | Cz1 | H |
| 559 | Cz1 | Cz1 | H | m-D1 | H |
| 560 | m-D1 | H | Cz1 | Cz1 | H |
| 561 | Cz1 | H | m-D1 | Cz1 | H |
| 562 | Cz1 | H | Cz1 | m-D1 | H |
| 563 | H | m-D1 | Cz1 | Cz1 | H |
| 564 | H | Cz1 | m-D1 | Cz1 | H |
| 565 | m-D1 | Cz1 | H | H | Cz1 |
| 566 | Cz1 | m-D1 | H | H | Cz1 |
| 567 | Cz1 | Cz1 | H | H | m-D1 |
| 568 | m-D1 | H | Cz1 | H | Cz1 |
| 569 | Cz1 | H | m-D1 | H | Cz1 |
| 570 | m-D1 | Cz1 | H | H | H |
| 571 | Cz1 | m-D1 | H | H | H |
| 572 | m-D1 | H | Cz1 | H | H |
| 573 | Cz1 | H | m-D1 | H | H |
| 574 | H | m-D1 | Cz1 | H | H |
| 575 | H | Cz1 | m-D1 | H | H |
| 576 | m-D1 | H | H | Cz1 | H |
| 577 | Cz1 | H | H | m-D1 | H |
| 578 | H | m-D1 | H | Cz1 | H |
| 579 | m-D1 | H | H | H | Cz1 |
| 580 | m-D1 | m-D1 | Cz1 | Cz1 | H |
| 581 | m-D1 | Cz1 | m-D1 | Cz1 | H |
| 582 | m-D1 | Cz1 | Cz1 | m-D1 | H |
| 583 | Cz1 | m-D1 | m-D1 | Cz1 | H |
| 584 | Cz1 | m-D1 | Cz1 | m-D1 | H |
| 585 | Cz1 | Cz1 | m-D1 | m-D1 | H |
| 586 | m-D1 | m-D1 | Cz1 | H | Cz1 |
| 587 | m-D1 | Cz1 | m-D1 | H | Cz1 |
| 588 | m-D1 | Cz1 | Cz1 | H | m-D1 |
| 589 | Cz1 | m-D1 | m-D1 | H | Cz1 |
| 590 | Cz1 | m-D1 | Cz1 | H | m-D1 |
| 591 | Cz1 | Cz1 | m-D1 | H | m-D1 |
| 592 | m-D1 | m-D1 | H | Cz1 | Cz1 |
| 593 | m-D1 | Cz1 | H | m-D1 | Cz1 |
| 594 | m-D1 | Cz1 | H | Cz1 | m-D1 |
| 595 | Cz1 | m-D1 | H | m-D1 | Cz1 |
| 596 | m-D1 | m-D1 | Cz1 | H | H |
| 597 | m-D1 | Cz1 | m-D1 | H | H |
| 598 | Cz1 | m-D1 | m-D1 | H | H |
| 599 | m-D1 | m-D1 | H | Cz1 | H |
| 600 | m-D1 | Cz1 | H | m-D1 | H |
| 601 | Cz1 | m-D1 | H | m-D1 | H |
| 602 | m-D1 | H | m-D1 | Cz1 | H |
| 603 | m-D1 | H | Cz1 | m-D1 | H |
| 604 | Cz1 | H | m-D1 | m-D1 | H |
| 605 | H | m-D1 | m-D1 | Cz1 | H |
| 606 | H | m-D1 | Cz1 | m-D1 | H |
| 607 | m-D1 | m-D1 | H | H | Cz1 |
| 608 | m-D1 | Cz1 | H | H | m-D1 |
| 609 | Cz1 | m-D1 | H | H | m-D1 |
| 610 | m-D1 | H | m-D1 | H | Cz1 |
| 611 | m-D1 | H | Cz1 | H | m-D1 |
| 612 | m-D1 | m-D1 | m-D1 | Cz1 | Cz1 |
| 613 | m-D1 | m-D1 | Cz1 | m-D1 | Cz1 |
| 614 | m-D1 | m-D1 | Cz1 | Cz1 | m-D1 |
| 615 | m-D1 | Cz1 | m-D1 | m-D1 | Cz1 |
| 616 | m-D1 | Cz1 | m-D1 | Cz1 | m-D1 |
| 617 | m-D1 | m-D1 | m-D1 | Cz1 | H |
| 618 | m-D1 | m-D1 | Cz1 | m-D1 | H |
| 619 | m-D1 | Cz1 | m-D1 | Cz1 | H |
| 620 | Cz1 | m-D1 | m-D1 | m-D1 | H |
| 621 | m-D1 | m-D1 | m-D1 | H | Cz1 |
| 622 | m-D1 | m-D1 | Cz1 | H | m-D1 |
| 623 | m-D1 | Cz1 | m-D1 | H | m-D1 |
| 624 | Cz1 | m-D1 | m-D1 | H | m-D1 |
| 625 | m-D1 | m-D1 | H | m-D1 | Cz1 |
| 626 | m-D1 | m-D1 | H | Cz1 | m-D1 |
| 627 | m-D1 | m-D1 | m-D1 | m-D1 | Cz1 |
| 628 | m-D1 | m-D1 | m-D1 | Cz1 | m-D1 |
| 629 | m-D1 | m-D1 | Cz1 | m-D1 | m-D1 |
| 630 | m-D2 | Cz1 | Cz1 | Cz1 | Cz1 |
| 631 | Cz1 | m-D2 | Cz1 | Cz1 | Cz1 |
| 632 | Cz1 | Cz1 | m-D2 | Cz1 | Cz1 |
| 633 | m-D2 | Cz1 | Cz1 | Cz1 | H |
| 634 | Cz1 | m-D2 | Cz1 | Cz1 | H |
| 635 | Cz1 | Cz1 | m-D2 | Cz1 | H |
| 636 | Cz1 | Cz1 | Cz1 | m-D2 | H |
| 637 | m-D2 | Cz1 | Cz1 | H | Cz1 |
| 638 | Cz1 | m-D2 | Cz1 | H | Cz1 |
| 639 | Cz1 | Cz1 | m-D2 | H | Cz1 |
| 640 | Cz1 | Cz1 | Cz1 | H | m-D2 |
| 641 | m-D2 | Cz1 | H | Cz1 | Cz1 |
| 642 | Cz1 | m-D2 | H | Cz1 | Cz1 |
| 643 | m-D2 | Cz1 | Cz1 | H | H |
| 644 | Cz1 | m-D2 | Cz1 | H | H |
| 645 | Cz1 | Cz1 | m-D2 | H | H |
| 646 | m-D2 | Cz1 | H | Cz1 | H |
| 647 | Cz1 | m-D2 | H | Cz1 | H |
| 648 | Cz1 | Cz1 | H | m-D2 | H |
| 649 | m-D2 | H | Cz1 | Cz1 | H |
| 650 | Cz1 | H | m-D2 | Cz1 | H |
| 651 | Cz1 | H | Cz1 | m-D2 | H |
| 652 | H | m-D2 | Cz1 | Cz1 | H |
| 653 | H | Cz1 | m-D2 | Cz1 | H |
| 654 | m-D2 | Cz1 | H | H | Cz1 |
| 655 | Cz1 | m-D2 | H | H | Cz1 |
| 656 | Cz1 | Cz1 | H | H | m-D2 |
| 657 | m-D2 | H | Cz1 | H | Cz1 |
| 658 | Cz1 | H | m-D2 | H | Cz1 |
| 659 | m-D2 | Cz1 | H | H | H |
| 660 | Cz1 | m-D2 | H | H | H |
| 661 | m-D2 | H | Cz1 | H | H |
| 662 | Cz1 | H | m-D2 | H | H |
| 663 | H | m-D2 | Cz1 | H | H |
| 664 | H | Cz1 | m-D2 | H | H |
| 665 | m-D2 | H | H | Cz1 | H |
| 666 | Cz1 | H | H | m-D2 | H |
| 667 | H | m-D2 | H | Cz1 | H |
| 668 | m-D2 | H | H | H | Cz1 |
| 669 | m-D2 | m-D2 | Cz1 | Cz1 | H |
| 670 | m-D2 | Cz1 | m-D2 | Cz1 | H |
| 671 | m-D2 | Cz1 | Cz1 | m-D2 | H |
| 672 | Cz1 | m-D2 | m-D2 | Cz1 | H |
| 673 | Cz1 | m-D2 | Cz1 | m-D2 | H |
| 674 | Cz1 | Cz1 | m-D2 | m-D2 | H |
| 675 | m-D2 | m-D2 | Cz1 | H | Cz1 |
| 676 | m-D2 | Cz1 | m-D2 | H | Cz1 |
| 677 | m-D2 | Cz1 | Cz1 | H | m-D2 |
| 678 | Cz1 | m-D2 | m-D2 | H | Cz1 |
| 679 | Cz1 | m-D2 | Cz1 | H | m-D2 |
| 680 | Cz1 | Cz1 | m-D2 | H | m-D2 |
| 681 | m-D2 | m-D2 | H | Cz1 | Cz1 |
| 682 | m-D2 | Cz1 | H | m-D2 | Cz1 |
| 683 | m-D2 | Cz1 | H | Cz1 | m-D2 |
| 684 | Cz1 | m-D2 | H | m-D2 | Cz1 |
| 685 | m-D2 | m-D2 | Cz1 | H | H |
| 686 | m-D2 | Cz1 | m-D2 | H | H |
| 687 | Cz1 | m-D2 | m-D2 | H | H |
| 688 | m-D2 | m-D2 | H | Cz1 | H |
| 689 | m-D2 | Cz1 | H | m-D2 | H |
| 690 | Cz1 | m-D2 | H | m-D2 | H |
| 691 | m-D2 | H | m-D2 | Cz1 | H |
| 692 | m-D2 | H | Cz1 | m-D2 | H |
| 693 | Cz1 | H | m-D2 | m-D2 | H |
| 694 | H | m-D2 | m-D2 | Cz1 | H |
| 695 | H | m-D2 | Cz1 | m-D2 | H |
| 696 | m-D2 | m-D2 | H | H | Cz1 |
| 697 | m-D2 | Cz1 | H | H | m-D2 |
| 698 | Cz1 | m-D2 | H | H | m-D2 |
| 699 | m-D2 | H | m-D2 | H | Cz1 |
| 700 | m-D2 | H | Cz1 | H | m-D2 |
| 701 | m-D2 | m-D2 | m-D2 | Cz1 | Cz1 |
| 702 | m-D2 | m-D2 | Cz1 | m-D2 | Cz1 |
| 703 | m-D2 | m-D2 | Cz1 | Cz1 | m-D2 |
| 704 | m-D2 | Cz1 | m-D2 | m-D2 | Cz1 |
| 705 | m-D2 | Cz1 | m-D2 | Cz1 | m-D2 |
| 706 | m-D2 | m-D2 | m-D2 | Cz1 | H |
| 707 | m-D2 | m-D2 | Cz1 | m-D2 | H |
| 708 | m-D2 | Cz1 | m-D2 | Cz1 | H |
| 709 | Cz1 | m-D2 | m-D2 | m-D2 | H |
| 710 | m-D2 | m-D2 | m-D2 | H | Cz1 |
| 711 | m-D2 | m-D2 | Cz1 | H | m-D2 |
| 712 | m-D2 | Cz1 | m-D2 | H | m-D2 |
| 713 | Cz1 | m-D2 | m-D2 | H | m-D2 |
| 714 | m-D2 | m-D2 | H | m-D2 | Cz1 |
| 715 | m-D2 | m-D2 | H | Cz1 | m-D2 |
| 716 | m-D2 | m-D2 | m-D2 | m-D2 | Cz1 |
| 717 | m-D2 | m-D2 | m-D2 | Cz1 | m-D2 |
| 718 | m-D2 | m-D2 | Cz1 | m-D2 | m-D2 |
| 719 | m-D3 | Cz1 | Cz1 | Cz1 | Cz1 |
| 720 | Cz1 | m-D3 | Cz1 | Cz1 | Cz1 |
| 721 | Cz1 | Cz1 | m-D3 | Cz1 | Cz1 |
| 722 | m-D3 | Cz1 | Cz1 | Cz1 | H |
| 723 | Cz1 | m-D3 | Cz1 | Cz1 | H |
| 724 | Cz1 | Cz1 | m-D3 | Cz1 | H |
| 725 | Cz1 | Cz1 | Cz1 | m-D3 | H |
| 726 | m-D3 | Cz1 | Cz1 | H | Cz1 |
| 727 | Cz1 | m-D3 | Cz1 | H | Cz1 |
| 728 | Cz1 | Cz1 | m-D3 | H | Cz1 |
| 729 | Cz1 | Cz1 | Cz1 | H | m-D3 |
| 730 | m-D3 | Cz1 | H | Cz1 | Cz1 |
| 731 | Cz1 | m-D3 | H | Cz1 | Cz1 |
| 732 | m-D3 | Cz1 | Cz1 | H | H |
| 733 | Cz1 | m-D3 | Cz1 | H | H |
| 734 | Cz1 | Cz1 | m-D3 | H | H |
| 735 | m-D3 | Cz1 | H | Cz1 | H |
| 736 | Cz1 | m-D3 | H | Cz1 | H |
| 737 | Cz1 | Cz1 | H | m-D3 | H |
| 738 | m-D3 | H | Cz1 | Cz1 | H |
| 739 | Cz1 | H | m-D3 | Cz1 | H |
| 740 | Cz1 | H | Cz1 | m-D3 | H |
| 741 | H | m-D3 | Cz1 | Cz1 | H |
| 742 | H | Cz1 | m-D3 | Cz1 | H |
| 743 | m-D3 | Cz1 | H | H | Cz1 |
| 744 | Cz1 | m-D3 | H | H | Cz1 |
| 745 | Cz1 | Cz1 | H | H | m-D3 |
| 746 | m-D3 | H | Cz1 | H | Cz1 |
| 747 | Cz1 | H | m-D3 | H | Cz1 |
| 748 | m-D3 | Cz1 | H | H | H |
| 749 | Cz1 | m-D3 | H | H | H |
| 750 | m-D3 | H | Cz1 | H | H |
| 751 | Cz1 | H | m-D3 | H | H |
| 752 | H | m-D3 | Cz1 | H | H |
| 753 | H | Cz1 | m-D3 | H | H |
| 754 | m-D3 | H | H | Cz1 | H |
| 755 | Cz1 | H | H | m-D3 | H |
| 756 | H | m-D3 | H | Cz1 | H |
| 757 | m-D3 | H | H | H | Cz1 |
| 758 | m-D3 | m-D3 | Cz1 | Cz1 | H |
| 759 | m-D3 | Cz1 | m-D3 | Cz1 | H |
| 760 | m-D3 | Cz1 | Cz1 | m-D3 | H |
| 761 | Cz1 | m-D3 | m-D3 | Cz1 | H |
| 762 | Cz1 | m-D3 | Cz1 | m-D3 | H |
| 763 | Cz1 | Cz1 | m-D3 | m-D3 | H |
| 764 | m-D3 | m-D3 | Cz1 | H | Cz1 |
| 765 | m-D3 | Cz1 | m-D3 | H | Cz1 |
| 766 | m-D3 | Cz1 | Cz1 | H | m-D3 |
| 767 | Cz1 | m-D3 | m-D3 | H | Cz1 |
| 768 | Cz1 | m-D3 | Cz1 | H | m-D3 |
| 769 | Cz1 | Cz1 | m-D3 | H | m-D3 |
| 770 | m-D3 | m-D3 | H | Cz1 | Cz1 |
| 771 | m-D3 | Cz1 | H | m-D3 | Cz1 |
| 772 | m-D3 | Cz1 | H | Cz1 | m-D3 |
| 773 | Cz1 | m-D3 | H | m-D3 | Cz1 |
| 774 | m-D3 | m-D3 | Cz1 | H | H |
| 775 | m-D3 | Cz1 | m-D3 | H | H |
| 776 | Cz1 | m-D3 | m-D3 | H | H |
| 777 | m-D3 | m-D3 | H | Cz1 | H |
| 778 | m-D3 | Cz1 | H | m-D3 | H |
| 779 | Cz1 | m-D3 | H | m-D3 | H |
| 780 | m-D3 | H | m-D3 | Cz1 | H |
| 781 | m-D3 | H | Cz1 | m-D3 | H |
| 782 | Cz1 | H | m-D3 | m-D3 | H |
| 783 | H | m-D3 | m-D3 | Cz1 | H |
| 784 | H | m-D3 | Cz1 | m-D3 | H |
| 785 | m-D3 | m-D3 | H | H | Cz1 |
| 786 | m-D3 | Cz1 | H | H | m-D3 |
| 787 | Cz1 | m-D3 | H | H | m-D3 |
| 788 | m-D3 | H | m-D3 | H | Cz1 |
| 789 | m-D3 | H | Cz1 | H | m-D3 |
| 790 | m-D3 | m-D3 | m-D3 | Cz1 | Cz1 |
| 791 | m-D3 | m-D3 | Cz1 | m-D3 | Cz1 |
| 792 | m-D3 | m-D3 | Cz1 | Cz1 | m-D3 |
| 793 | m-D3 | Cz1 | m-D3 | m-D3 | Cz1 |
| 794 | m-D3 | Cz1 | m-D3 | Cz1 | m-D3 |
| 795 | m-D3 | m-D3 | m-D3 | Cz1 | H |
| 796 | m-D3 | m-D3 | Cz1 | m-D3 | H |
| 797 | m-D3 | Cz1 | m-D3 | Cz1 | H |
| 798 | Cz1 | m-D3 | m-D3 | m-D3 | H |
| 799 | m-D3 | m-D3 | m-D3 | H | Cz1 |
| 800 | m-D3 | m-D3 | Cz1 | H | m-D3 |
| 801 | m-D3 | Cz1 | m-D3 | H | m-D3 |
| 802 | Cz1 | m-D3 | m-D3 | H | m-D3 |
| 803 | m-D3 | m-D3 | H | m-D3 | Cz1 |
| 804 | m-D3 | m-D3 | H | Cz1 | m-D3 |
| 805 | m-D3 | m-D3 | m-D3 | m-D3 | Cz1 |
| 806 | m-D3 | m-D3 | m-D3 | Cz1 | m-D3 |
| 807 | m-D3 | m-D3 | Cz1 | m-D3 | m-D3 |
| 808 | m-D1 | Cz2 | Cz2 | Cz2 | Cz2 |
| 809 | Cz2 | m-D1 | Cz2 | Cz2 | Cz2 |
| 810 | Cz2 | Cz2 | m-D1 | Cz2 | Cz2 |
| 811 | m-D1 | Cz2 | Cz2 | Cz2 | H |
| 812 | Cz2 | m-D1 | Cz2 | Cz2 | H |
| 813 | Cz2 | Cz2 | m-D1 | Cz2 | H |
| 814 | Cz2 | Cz2 | Cz2 | m-D1 | H |
| 815 | m-D1 | Cz2 | Cz2 | H | Cz2 |
| 816 | Cz2 | m-D1 | Cz2 | H | Cz2 |
| 817 | Cz2 | Cz2 | m-D1 | H | Cz2 |
| 818 | Cz2 | Cz2 | Cz2 | H | m-D1 |
| 819 | m-D1 | Cz2 | H | Cz2 | Cz2 |
| 820 | Cz2 | m-D1 | H | Cz2 | Cz2 |
| 821 | m-D1 | Cz2 | Cz2 | H | H |
| 822 | Cz2 | m-D1 | Cz2 | H | H |
| 823 | Cz2 | Cz2 | m-D1 | H | H |
| 824 | m-D1 | Cz2 | H | Cz2 | H |
| 825 | Cz2 | m-D1 | H | Cz2 | H |
| 826 | Cz2 | Cz2 | H | m-D1 | H |
| 827 | m-D1 | H | Cz2 | Cz2 | H |
| 828 | Cz2 | H | m-D1 | Cz2 | H |
| 829 | Cz2 | H | Cz2 | m-D1 | H |
| 830 | H | m-D1 | Cz2 | Cz2 | H |
| 831 | H | Cz2 | m-D1 | Cz2 | H |
| 832 | m-D1 | Cz2 | H | H | Cz2 |
| 833 | Cz2 | m-D1 | H | H | Cz2 |
| 834 | Cz2 | Cz2 | H | H | m-D1 |
| 835 | m-D1 | H | Cz2 | H | Cz2 |
| 836 | Cz2 | H | m-D1 | H | Cz2 |
| 837 | m-D1 | Cz2 | H | H | H |
| 838 | Cz2 | m-D1 | H | H | H |
| 839 | m-D1 | H | Cz2 | H | H |
| 840 | Cz2 | H | m-D1 | H | H |
| 841 | H | m-D1 | Cz2 | H | H |
| 842 | H | Cz2 | m-D1 | H | H |
| 843 | m-D1 | H | H | Cz2 | H |
| 844 | Cz2 | H | H | m-D1 | H |
| 845 | H | m-D1 | H | Cz2 | H |
| 846 | m-D1 | H | H | H | Cz2 |
| 847 | m-D1 | m-D1 | Cz2 | Cz2 | H |
| 848 | m-D1 | Cz2 | m-D1 | Cz2 | H |
| 849 | m-D1 | Cz2 | Cz2 | m-D1 | H |
| 850 | Cz2 | m-D1 | m-D1 | Cz2 | H |
| 851 | Cz2 | m-D1 | Cz2 | m-D1 | H |
| 852 | Cz2 | Cz2 | m-D1 | m-D1 | H |
| 853 | m-D1 | m-D1 | Cz2 | H | Cz2 |
| 854 | m-D1 | Cz2 | m-D1 | H | Cz2 |
| 855 | m-D1 | Cz2 | Cz2 | H | m-D1 |
| 856 | Cz2 | m-D1 | m-D1 | H | Cz2 |
| 857 | Cz2 | m-D1 | Cz2 | H | m-D1 |
| 858 | Cz2 | Cz2 | m-D1 | H | m-D1 |
| 859 | m-D1 | m-D1 | H | Cz2 | Cz2 |
| 860 | m-D1 | Cz2 | H | m-D1 | Cz2 |
| 861 | m-D1 | Cz2 | H | Cz2 | m-D1 |
| 862 | Cz2 | m-D1 | H | m-D1 | Cz2 |
| 863 | m-D1 | m-D1 | Cz2 | H | H |
| 864 | m-D1 | Cz2 | m-D1 | H | H |
| 865 | Cz2 | m-D1 | m-D1 | H | H |
| 866 | m-D1 | m-D1 | H | Cz2 | H |
| 867 | m-D1 | Cz2 | H | m-D1 | H |
| 868 | Cz2 | m-D1 | H | m-D1 | H |
| 869 | m-D1 | H | m-D1 | Cz2 | H |
| 870 | m-D1 | H | Cz2 | m-D1 | H |
| 871 | Cz2 | H | m-D1 | m-D1 | H |
| 872 | H | m-D1 | m-D1 | Cz2 | H |
| 873 | H | m-D1 | Cz2 | m-D1 | H |
| 874 | m-D1 | m-D1 | H | H | Cz2 |
| 875 | m-D1 | Cz2 | H | H | m-D1 |
| 876 | Cz2 | m-D1 | H | H | m-D1 |
| 877 | m-D1 | H | m-D1 | H | Cz2 |
| 878 | m-D1 | H | Cz2 | H | m-D1 |
| 879 | m-D1 | m-D1 | m-D1 | Cz2 | Cz2 |
| 880 | m-D1 | m-D1 | Cz2 | m-D1 | Cz2 |
| 881 | m-D1 | m-D1 | Cz2 | Cz2 | m-D1 |
| 882 | m-D1 | Cz2 | m-D1 | m-D1 | Cz2 |
| 883 | m-D1 | Cz2 | m-D1 | Cz2 | m-D1 |
| 884 | m-D1 | m-D1 | m-D1 | Cz2 | H |
| 885 | m-D1 | m-D1 | Cz2 | m-D1 | H |
| 886 | m-D1 | Cz2 | m-D1 | Cz2 | H |
| 887 | Cz2 | m-D1 | m-D1 | m-D1 | H |
| 888 | m-D1 | m-D1 | m-D1 | H | Cz2 |
| 889 | m-D1 | m-D1 | Cz2 | H | m-D1 |
| 890 | m-D1 | Cz2 | m-D1 | H | m-D1 |
| 891 | Cz2 | m-D1 | m-D1 | H | m-D1 |
| 892 | m-D1 | m-D1 | H | m-D1 | Cz2 |
| 893 | m-D1 | m-D1 | H | Cz2 | m-D1 |
| 894 | m-D1 | m-D1 | m-D1 | m-D1 | Cz2 |
| 895 | m-D1 | m-D1 | m-D1 | Cz2 | m-D1 |
| 896 | m-D1 | m-D1 | Cz2 | m-D1 | m-D1 |
| 897 | m-D2 | Cz2 | Cz2 | Cz2 | Cz2 |
| 898 | Cz2 | m-D2 | Cz2 | Cz2 | Cz2 |
| 899 | Cz2 | Cz2 | m-D2 | Cz2 | Cz2 |
| 900 | m-D2 | Cz2 | Cz2 | Cz2 | H |
| 901 | Cz2 | m-D2 | Cz2 | Cz2 | H |
| 902 | Cz2 | Cz2 | m-D2 | Cz2 | H |
| 903 | Cz2 | Cz2 | Cz2 | m-D2 | H |
| 904 | m-D2 | Cz2 | Cz2 | H | Cz2 |
| 905 | Cz2 | m-D2 | Cz2 | H | Cz2 |
| 906 | Cz2 | Cz2 | m-D2 | H | Cz2 |
| 907 | Cz2 | Cz2 | Cz2 | H | m-D2 |
| 908 | m-D2 | Cz2 | H | Cz2 | Cz2 |
| 909 | Cz2 | m-D2 | H | Cz2 | Cz2 |
| 910 | m-D2 | Cz2 | Cz2 | H | H |
| 911 | Cz2 | m-D2 | Cz2 | H | H |
| 912 | Cz2 | Cz2 | m-D2 | H | H |
| 913 | m-D2 | Cz2 | H | Cz2 | H |
| 914 | Cz2 | m-D2 | H | Cz2 | H |
| 915 | Cz2 | Cz2 | H | m-D2 | H |
| 916 | m-D2 | H | Cz2 | Cz2 | H |
| 917 | Cz2 | H | m-D2 | Cz2 | H |
| 918 | Cz2 | H | Cz2 | m-D2 | H |
| 919 | H | m-D2 | Cz2 | Cz2 | H |
| 920 | H | Cz2 | m-D2 | Cz2 | H |
| 921 | m-D2 | Cz2 | H | H | Cz2 |
| 922 | Cz2 | m-D2 | H | H | Cz2 |
| 923 | Cz2 | Cz2 | H | H | m-D2 |
| 924 | m-D2 | H | Cz2 | H | Cz2 |
| 925 | Cz2 | H | m-D2 | H | Cz2 |
| 926 | m-D2 | Cz2 | H | H | H |
| 927 | Cz2 | m-D2 | H | H | H |
| 928 | m-D2 | H | Cz2 | H | H |
| 929 | Cz2 | H | m-D2 | H | H |
| 930 | H | m-D2 | Cz2 | H | H |
| 931 | H | Cz2 | m-D2 | H | H |
| 932 | m-D2 | H | H | Cz2 | H |
| 933 | Cz2 | H | H | m-D2 | H |
| 934 | H | m-D2 | H | Cz2 | H |
| 935 | m-D2 | H | H | H | Cz2 |
| 936 | m-D2 | m-D2 | Cz2 | Cz2 | H |
| 937 | m-D2 | Cz2 | m-D2 | Cz2 | H |
| 938 | m-D2 | Cz2 | Cz2 | m-D2 | H |
| 939 | Cz2 | m-D2 | m-D2 | Cz2 | H |
| 940 | Cz2 | m-D2 | Cz2 | m-D2 | H |
| 941 | Cz2 | Cz2 | m-D2 | m-D2 | H |
| 942 | m-D2 | m-D2 | Cz2 | H | Cz2 |
| 943 | m-D2 | Cz2 | m-D2 | H | Cz2 |
| 944 | m-D2 | Cz2 | Cz2 | H | m-D2 |
| 945 | Cz2 | m-D2 | m-D2 | H | Cz2 |
| 946 | Cz2 | m-D2 | Cz2 | H | m-D2 |
| 947 | Cz2 | Cz2 | m-D2 | H | m-D2 |
| 948 | m-D2 | m-D2 | H | Cz2 | Cz2 |
| 949 | m-D2 | Cz2 | H | m-D2 | Cz2 |
| 950 | m-D2 | Cz2 | H | Cz2 | m-D2 |
| 951 | Cz2 | m-D2 | H | m-D2 | Cz2 |
| 952 | m-D2 | m-D2 | Cz2 | H | H |
| 953 | m-D2 | Cz2 | m-D2 | H | H |
| 954 | Cz2 | m-D2 | m-D2 | H | H |
| 955 | m-D2 | m-D2 | H | Cz2 | H |
| 956 | m-D2 | Cz2 | H | m-D2 | H |
| 957 | Cz2 | m-D2 | H | m-D2 | H |
| 958 | m-D2 | H | m-D2 | Cz2 | H |
| 959 | m-D2 | H | Cz2 | m-D2 | H |
| 960 | Cz2 | H | m-D2 | m-D2 | H |
| 961 | H | m-D2 | m-D2 | Cz2 | H |
| 962 | H | m-D2 | Cz2 | m-D2 | H |
| 963 | m-D2 | m-D2 | H | H | Cz2 |
| 964 | m-D2 | Cz2 | H | H | m-D2 |
| 965 | Cz2 | m-D2 | H | H | m-D2 |
| 966 | m-D2 | H | m-D2 | H | Cz2 |
| 967 | m-D2 | H | Cz2 | H | m-D2 |
| 968 | m-D2 | m-D2 | m-D2 | Cz2 | Cz2 |
| 969 | m-D2 | m-D2 | Cz2 | m-D2 | Cz2 |
| 970 | m-D2 | m-D2 | Cz2 | Cz2 | m-D2 |
| 971 | m-D2 | Cz2 | m-D2 | m-D2 | Cz2 |
| 972 | m-D2 | Cz2 | m-D2 | Cz2 | m-D2 |
| 973 | m-D2 | m-D2 | m-D2 | Cz2 | H |
| 974 | m-D2 | m-D2 | Cz2 | m-D2 | H |
| 975 | m-D2 | Cz2 | m-D2 | Cz2 | H |
| 976 | Cz2 | m-D2 | m-D2 | m-D2 | H |
| 977 | m-D2 | m-D2 | m-D2 | H | Cz2 |
| 978 | m-D2 | m-D2 | Cz2 | H | m-D2 |
| 979 | m-D2 | Cz2 | m-D2 | H | m-D2 |
| 980 | Cz2 | m-D2 | m-D2 | H | m-D2 |
| 981 | m-D2 | m-D2 | H | m-D2 | Cz2 |
| 982 | m-D2 | m-D2 | H | Cz2 | m-D2 |
| 983 | m-D2 | m-D2 | m-D2 | m-D2 | Cz2 |
| 984 | m-D2 | m-D2 | m-D2 | Cz2 | m-D2 |
| 985 | m-D2 | m-D2 | Cz2 | m-D2 | m-D2 |
| 986 | m-D3 | Cz2 | Cz2 | Cz2 | Cz2 |
| 987 | Cz2 | m-D3 | Cz2 | Cz2 | Cz2 |
| 988 | Cz2 | Cz2 | m-D3 | Cz2 | Cz2 |
| 989 | m-D3 | Cz2 | Cz2 | Cz2 | H |
| 990 | Cz2 | m-D3 | Cz2 | Cz2 | H |
| 991 | Cz2 | Cz2 | m-D3 | Cz2 | H |
| 992 | Cz2 | Cz2 | Cz2 | m-D3 | H |
| 993 | m-D3 | Cz2 | Cz2 | H | Cz2 |
| 994 | Cz2 | m-D3 | Cz2 | H | Cz2 |
| 995 | Cz2 | Cz2 | m-D3 | H | Cz2 |
| 996 | Cz2 | Cz2 | Cz2 | H | m-D3 |
| 997 | m-D3 | Cz2 | H | Cz2 | Cz2 |
| 998 | Cz2 | m-D3 | H | Cz2 | Cz2 |
| 999 | m-D3 | Cz2 | Cz2 | H | H |
| 1000 | Cz2 | m-D3 | Cz2 | H | H |
| 1001 | Cz2 | Cz2 | m-D3 | H | H |
| 1002 | m-D3 | Cz2 | H | Cz2 | H |
| 1003 | Cz2 | m-D3 | H | Cz2 | H |
| 1004 | Cz2 | Cz2 | H | m-D3 | H |
| 1005 | m-D3 | H | Cz2 | Cz2 | H |
| 1006 | Cz2 | H | m-D3 | Cz2 | H |
| 1007 | Cz2 | H | Cz2 | m-D3 | H |
| 1008 | H | m-D3 | Cz2 | Cz2 | H |
| 1009 | H | Cz2 | m-D3 | Cz2 | H |
| 1010 | m-D3 | Cz2 | H | H | Cz2 |
| 1011 | Cz2 | m-D3 | H | H | Cz2 |
| 1012 | Cz2 | Cz2 | H | H | m-D3 |
| 1013 | m-D3 | H | Cz2 | H | Cz2 |
| 1014 | Cz2 | H | m-D3 | H | Cz2 |
| 1015 | m-D3 | Cz2 | H | H | H |
| 1016 | Cz2 | m-D3 | H | H | H |
| 1017 | m-D3 | H | Cz2 | H | H |
| 1018 | Cz2 | H | m-D3 | H | H |
| 1019 | H | m-D3 | Cz2 | H | H |
| 1020 | H | Cz2 | m-D3 | H | H |
| 1021 | m-D3 | H | H | Cz2 | H |
| 1022 | Cz2 | H | H | m-D3 | H |
| 1023 | H | m-D3 | H | Cz2 | H |
| 1024 | m-D3 | H | H | H | Cz2 |
| 1025 | m-D3 | m-D3 | Cz2 | Cz2 | H |
| 1026 | m-D3 | Cz2 | m-D3 | Cz2 | H |
| 1027 | m-D3 | Cz2 | Cz2 | m-D3 | H |
| 1028 | Cz2 | m-D3 | m-D3 | Cz2 | H |
| 1029 | Cz2 | m-D3 | Cz2 | m-D3 | H |
| 1030 | Cz2 | Cz2 | m-D3 | m-D3 | H |
| 1031 | m-D3 | m-D3 | Cz2 | H | Cz2 |
| 1032 | m-D3 | Cz2 | m-D3 | H | Cz2 |
| 1033 | m-D3 | Cz2 | Cz2 | H | m-D3 |
| 1034 | Cz2 | m-D3 | m-D3 | H | Cz2 |
| 1035 | Cz2 | m-D3 | Cz2 | H | m-D3 |
| 1036 | Cz2 | Cz2 | m-D3 | H | m-D3 |
| 1037 | m-D3 | m-D3 | H | Cz2 | Cz2 |
| 1038 | m-D3 | Cz2 | H | m-D3 | Cz2 |
| 1039 | m-D3 | Cz2 | H | Cz2 | m-D3 |
| 1040 | Cz2 | m-D3 | H | m-D3 | Cz2 |
| 1041 | m-D3 | m-D3 | Cz2 | H | H |
| 1042 | m-D3 | Cz2 | m-D3 | H | H |
| 1043 | Cz2 | m-D3 | m-D3 | H | H |
| 1044 | m-D3 | m-D3 | H | Cz2 | H |
| 1045 | m-D3 | Cz2 | H | m-D3 | H |
| 1046 | Cz2 | m-D3 | H | m-D3 | H |
| 1047 | m-D3 | H | m-D3 | Cz2 | H |
| 1048 | m-D3 | H | Cz2 | m-D3 | H |
| 1049 | Cz2 | H | m-D3 | m-D3 | H |
| 1050 | H | m-D3 | m-D3 | Cz2 | H |
| 1051 | H | m-D3 | Cz2 | m-D3 | H |
| 1052 | m-D3 | m-D3 | H | H | Cz2 |
| 1053 | m-D3 | Cz2 | H | H | m-D3 |
| 1054 | Cz2 | m-D3 | H | H | m-D3 |
| 1055 | m-D3 | H | m-D3 | H | Cz2 |
| 1056 | m-D3 | H | Cz2 | H | m-D3 |
| 1057 | m-D3 | m-D3 | m-D3 | Cz2 | Cz2 |
| 1058 | m-D3 | m-D3 | Cz2 | m-D3 | Cz2 |
| 1059 | m-D3 | m-D3 | Cz2 | Cz2 | m-D3 |
| 1060 | m-D3 | Cz2 | m-D3 | m-D3 | Cz2 |
| 1061 | m-D3 | Cz2 | m-D3 | Cz2 | m-D3 |
| 1062 | m-D3 | m-D3 | m-D3 | Cz2 | H |
| 1063 | m-D3 | m-D3 | Cz2 | m-D3 | H |
| 1064 | m-D3 | Cz2 | m-D3 | Cz2 | H |
| 1065 | Cz2 | m-D3 | m-D3 | m-D3 | H |
| 1066 | m-D3 | m-D3 | m-D3 | H | Cz2 |
| 1067 | m-D3 | m-D3 | Cz2 | H | m-D3 |
| 1068 | m-D3 | Cz2 | m-D3 | H | m-D3 |
| 1069 | Cz2 | m-D3 | m-D3 | H | m-D3 |
| 1070 | m-D3 | m-D3 | H | m-D3 | Cz2 |
| 1071 | m-D3 | m-D3 | H | Cz2 | m-D3 |
| 1072 | m-D3 | m-D3 | m-D3 | m-D3 | Cz2 |
| 1073 | m-D3 | m-D3 | m-D3 | Cz2 | m-D3 |
| 1074 | m-D3 | m-D3 | Cz2 | m-D3 | m-D3 |
| 1075 | m-D1 | Cz3 | Cz3 | Cz3 | Cz3 |
| 1076 | Cz3 | m-D1 | Cz3 | Cz3 | Cz3 |
| 1077 | Cz3 | Cz3 | m-D1 | Cz3 | Cz3 |
| 1078 | m-D1 | Cz3 | Cz3 | Cz3 | H |
| 1079 | Cz3 | m-D1 | Cz3 | Cz3 | H |
| 1080 | Cz3 | Cz3 | m-D1 | Cz3 | H |
| 1081 | Cz3 | Cz3 | Cz3 | m-D1 | H |
| 1082 | m-D1 | Cz3 | Cz3 | H | Cz3 |
| 1083 | Cz3 | m-D1 | Cz3 | H | Cz3 |
| 1084 | Cz3 | Cz3 | m-D1 | H | Cz3 |
| 1085 | Cz3 | Cz3 | Cz3 | H | m-D1 |
| 1086 | m-D1 | Cz3 | H | Cz3 | Cz3 |
| 1087 | Cz3 | m-D1 | H | Cz3 | Cz3 |
| 1088 | m-D1 | Cz3 | Cz3 | H | H |
| 1089 | Cz3 | m-D1 | Cz3 | H | H |
| 1090 | Cz3 | Cz3 | m-D1 | H | H |
| 1091 | m-D1 | Cz3 | H | Cz3 | H |
| 1092 | Cz3 | m-D1 | H | Cz3 | H |
| 1093 | Cz3 | Cz3 | H | m-D1 | H |
| 1094 | m-D1 | H | Cz3 | Cz3 | H |
| 1095 | Cz3 | H | m-D1 | Cz3 | H |
| 1096 | Cz3 | H | Cz3 | m-D1 | H |
| 1097 | H | m-D1 | Cz3 | Cz3 | H |
| 1098 | H | Cz3 | m-D1 | Cz3 | H |
| 1099 | m-D1 | Cz3 | H | H | Cz3 |
| 1100 | Cz3 | m-D1 | H | H | Cz3 |
| 1101 | Cz3 | Cz3 | H | H | m-D1 |
| 1102 | m-D1 | H | Cz3 | H | Cz3 |
| 1103 | Cz3 | H | m-D1 | H | Cz3 |
| 1104 | m-D1 | Cz3 | H | H | H |
| 1105 | Cz3 | m-D1 | H | H | H |
| 1106 | m-D1 | H | Cz3 | H | H |
| 1107 | Cz3 | H | m-D1 | H | H |
| 1108 | H | m-D1 | Cz3 | H | H |
| 1109 | H | Cz3 | m-D1 | H | H |
| 1110 | m-D1 | H | H | Cz3 | H |
| 1111 | Cz3 | H | H | m-D1 | H |
| 1112 | H | m-D1 | H | Cz3 | H |
| 1113 | m-D1 | H | H | H | Cz3 |
| 1114 | m-D1 | m-D1 | Cz3 | Cz3 | H |
| 1115 | m-D1 | Cz3 | m-D1 | Cz3 | H |
| 1116 | m-D1 | Cz3 | Cz3 | m-D1 | H |
| 1117 | Cz3 | m-D1 | m-D1 | Cz3 | H |
| 1118 | Cz3 | m-D1 | Cz3 | m-D1 | H |
| 1119 | Cz3 | Cz3 | m-D1 | m-D1 | H |
| 1120 | m-D1 | m-D1 | Cz3 | H | Cz3 |
| 1121 | m-D1 | Cz3 | m-D1 | H | Cz3 |
| 1122 | m-D1 | Cz3 | Cz3 | H | m-D1 |
| 1123 | Cz3 | m-D1 | m-D1 | H | Cz3 |
| 1124 | Cz3 | m-D1 | Cz3 | H | m-D1 |
| 1125 | Cz3 | Cz3 | m-D1 | H | m-D1 |
| 1126 | m-D1 | m-D1 | H | Cz3 | Cz3 |
| 1127 | m-D1 | Cz3 | H | m-D1 | Cz3 |
| 1128 | m-D1 | Cz3 | H | Cz3 | m-D1 |
| 1129 | Cz3 | m-D1 | H | m-D1 | Cz3 |
| 1130 | m-D1 | m-D1 | Cz3 | H | H |
| 1131 | m-D1 | Cz3 | m-D1 | H | H |
| 1132 | Cz3 | m-D1 | m-D1 | H | H |
| 1133 | m-D1 | m-D1 | H | Cz3 | H |
| 1134 | m-D1 | Cz3 | H | m-D1 | H |
| 1135 | Cz3 | m-D1 | H | m-D1 | H |
| 1136 | m-D1 | H | m-D1 | Cz3 | H |
| 1137 | m-D1 | H | Cz3 | m-D1 | H |
| 1138 | Cz3 | H | m-D1 | m-D1 | H |
| 1139 | H | m-D1 | m-D1 | Cz3 | H |
| 1140 | H | m-D1 | Cz3 | m-D1 | H |
| 1141 | m-D1 | m-D1 | H | H | Cz3 |
| 1142 | m-D1 | Cz3 | H | H | m-D1 |
| 1143 | Cz3 | m-D1 | H | H | m-D1 |
| 1144 | m-D1 | H | m-D1 | H | Cz3 |
| 1145 | m-D1 | H | Cz3 | H | m-D1 |
| 1146 | m-D1 | m-D1 | m-D1 | Cz3 | Cz3 |
| 1147 | m-D1 | m-D1 | Cz3 | m-D1 | Cz3 |
| 1148 | m-D1 | m-D1 | Cz3 | Cz3 | m-D1 |
| 1149 | m-D1 | Cz3 | m-D1 | m-D1 | Cz3 |
| 1150 | m-D1 | Cz3 | m-D1 | Cz3 | m-D1 |
| 1151 | m-D1 | m-D1 | m-D1 | Cz3 | H |
| 1152 | m-D1 | m-D1 | Cz3 | m-D1 | H |
| 1153 | m-D1 | Cz3 | m-D1 | Cz3 | H |
| 1154 | Cz3 | m-D1 | m-D1 | m-D1 | H |
| 1155 | m-D1 | m-D1 | m-D1 | H | Cz3 |
| 1156 | m-D1 | m-D1 | Cz3 | H | m-D1 |
| 1157 | m-D1 | Cz3 | m-D1 | H | m-D1 |
| 1158 | Cz3 | m-D1 | m-D1 | H | m-D1 |
| 1159 | m-D1 | m-D1 | H | m-D1 | Cz3 |
| 1160 | m-D1 | m-D1 | H | Cz3 | m-D1 |
| 1161 | m-D1 | m-D1 | m-D1 | m-D1 | Cz3 |
| 1162 | m-D1 | m-D1 | m-D1 | Cz3 | m-D1 |
| 1163 | m-D1 | m-D1 | Cz3 | m-D1 | m-D1 |
| 1164 | m-D2 | Cz3 | Cz3 | Cz3 | Cz3 |
| 1165 | Cz3 | m-D2 | Cz3 | Cz3 | Cz3 |
| 1166 | Cz3 | Cz3 | m-D2 | Cz3 | Cz3 |
| 1167 | m-D2 | Cz3 | Cz3 | Cz3 | H |
| 1168 | Cz3 | m-D2 | Cz3 | Cz3 | H |
| 1169 | Cz3 | Cz3 | m-D2 | Cz3 | H |
| 1170 | Cz3 | Cz3 | Cz3 | m-D2 | H |
| 1171 | m-D2 | Cz3 | Cz3 | H | Cz3 |
| 1172 | Cz3 | m-D2 | Cz3 | H | Cz3 |
| 1173 | Cz3 | Cz3 | m-D2 | H | Cz3 |
| 1174 | Cz3 | Cz3 | Cz3 | H | m-D2 |
| 1175 | m-D2 | Cz3 | H | Cz3 | Cz3 |
| 1176 | Cz3 | m-D2 | H | Cz3 | Cz3 |
| 1177 | m-D2 | Cz3 | Cz3 | H | H |
| 1178 | Cz3 | m-D2 | Cz3 | H | H |
| 1179 | Cz3 | Cz3 | m-D2 | H | H |
| 1180 | m-D2 | Cz3 | H | Cz3 | H |
| 1181 | Cz3 | m-D2 | H | Cz3 | H |
| 1182 | Cz3 | Cz3 | H | m-D2 | H |
| 1183 | m-D2 | H | Cz3 | Cz3 | H |
| 1184 | Cz3 | H | m-D2 | Cz3 | H |
| 1185 | Cz3 | H | Cz3 | m-D2 | H |
| 1186 | H | m-D2 | Cz3 | Cz3 | H |
| 1187 | H | Cz3 | m-D2 | Cz3 | H |
| 1188 | m-D2 | Cz3 | H | H | Cz3 |
| 1189 | Cz3 | m-D2 | H | H | Cz3 |
| 1190 | Cz3 | Cz3 | H | H | m-D2 |
| 1191 | m-D2 | H | Cz3 | H | Cz3 |
| 1192 | Cz3 | H | m-D2 | H | Cz3 |
| 1193 | m-D2 | Cz3 | H | H | H |
| 1194 | Cz3 | m-D2 | H | H | H |
| 1195 | m-D2 | H | Cz3 | H | H |
| 1196 | Cz3 | H | m-D2 | H | H |
| 1197 | H | m-D2 | Cz3 | H | H |
| 1198 | H | Cz3 | m-D2 | H | H |
| 1199 | m-D2 | H | H | Cz3 | H |
| 1200 | Cz3 | H | H | m-D2 | H |
| 1201 | H | m-D2 | H | Cz3 | H |
| 1202 | m-D2 | H | H | H | Cz3 |
| 1203 | m-D2 | m-D2 | Cz3 | Cz3 | H |
| 1204 | m-D2 | Cz3 | m-D2 | Cz3 | H |
| 1205 | m-D2 | Cz3 | Cz3 | m-D2 | H |
| 1206 | Cz3 | m-D2 | m-D2 | Cz3 | H |
| 1207 | Cz3 | m-D2 | Cz3 | m-D2 | H |
| 1208 | Cz3 | Cz3 | m-D2 | m-D2 | H |
| 1209 | m-D2 | m-D2 | Cz3 | H | Cz3 |
| 1210 | m-D2 | Cz3 | m-D2 | H | Cz3 |
| 1211 | m-D2 | Cz3 | Cz3 | H | m-D2 |
| 1212 | Cz3 | m-D2 | m-D2 | H | Cz3 |
| 1213 | Cz3 | m-D2 | Cz3 | H | m-D2 |
| 1214 | Cz3 | Cz3 | m-D2 | H | m-D2 |
| 1215 | m-D2 | m-D2 | H | Cz3 | Cz3 |
| 1216 | m-D2 | Cz3 | H | m-D2 | Cz3 |
| 1217 | m-D2 | Cz3 | H | Cz3 | m-D2 |
| 1218 | Cz3 | m-D2 | H | m-D2 | Cz3 |
| 1219 | m-D2 | m-D2 | Cz3 | H | H |
| 1220 | m-D2 | Cz3 | m-D2 | H | H |
| 1221 | Cz3 | m-D2 | m-D2 | H | H |
| 1222 | m-D2 | m-D2 | H | Cz3 | H |
| 1223 | m-D2 | Cz3 | H | m-D2 | H |
| 1224 | Cz3 | m-D2 | H | m-D2 | H |
| 1225 | m-D2 | H | m-D2 | Cz3 | H |
| 1226 | m-D2 | H | Cz3 | m-D2 | H |
| 1227 | Cz3 | H | m-D2 | m-D2 | H |
| 1228 | H | m-D2 | m-D2 | Cz3 | H |
| 1229 | H | m-D2 | Cz3 | m-D2 | H |
| 1230 | m-D2 | m-D2 | H | H | Cz3 |
| 1231 | m-D2 | Cz3 | H | H | m-D2 |
| 1232 | Cz3 | m-D2 | H | H | m-D2 |
| 1233 | m-D2 | H | m-D2 | H | Cz3 |
| 1234 | m-D2 | H | Cz3 | H | m-D2 |
| 1235 | m-D2 | m-D2 | m-D2 | Cz3 | Cz3 |
| 1236 | m-D2 | m-D2 | Cz3 | m-D2 | Cz3 |
| 1237 | m-D2 | m-D2 | Cz3 | Cz3 | m-D2 |
| 1238 | m-D2 | Cz3 | m-D2 | m-D2 | Cz3 |
| 1239 | m-D2 | Cz3 | m-D2 | Cz3 | m-D2 |
| 1240 | m-D2 | m-D2 | m-D2 | Cz3 | H |
| 1241 | m-D2 | m-D2 | Cz3 | m-D2 | H |
| 1242 | m-D2 | Cz3 | m-D2 | Cz3 | H |
| 1243 | Cz3 | m-D2 | m-D2 | m-D2 | H |
| 1244 | m-D2 | m-D2 | m-D2 | H | Cz3 |
| 1245 | m-D2 | m-D2 | Cz3 | H | m-D2 |
| 1246 | m-D2 | Cz3 | m-D2 | H | m-D2 |
| 1247 | Cz3 | m-D2 | m-D2 | H | m-D2 |
| 1248 | m-D2 | m-D2 | H | m-D2 | Cz3 |
| 1249 | m-D2 | m-D2 | H | Cz3 | m-D2 |
| 1250 | m-D2 | m-D2 | m-D2 | m-D2 | Cz3 |
| 1251 | m-D2 | m-D2 | m-D2 | Cz3 | m-D2 |
| 1252 | m-D2 | m-D2 | Cz3 | m-D2 | m-D2 |
| 1253 | m-D3 | Cz3 | Cz3 | Cz3 | Cz3 |
| 1254 | Cz3 | m-D3 | Cz3 | Cz3 | Cz3 |
| 1255 | Cz3 | Cz3 | m-D3 | Cz3 | Cz3 |
| 1256 | m-D3 | Cz3 | Cz3 | Cz3 | H |
| 1257 | Cz3 | m-D3 | Cz3 | Cz3 | H |
| 1258 | Cz3 | Cz3 | m-D3 | Cz3 | H |
| 1259 | Cz3 | Cz3 | Cz3 | m-D3 | H |
| 1260 | m-D3 | Cz3 | Cz3 | H | Cz3 |
| 1261 | Cz3 | m-D3 | Cz3 | H | Cz3 |
| 1262 | Cz3 | Cz3 | m-D3 | H | Cz3 |
| 1263 | Cz3 | Cz3 | Cz3 | H | m-D3 |
| 1264 | m-D3 | Cz3 | H | Cz3 | Cz3 |
| 1265 | Cz3 | m-D3 | H | Cz | Cz3 |
| 1266 | m-D3 | Cz3 | Cz3 | H | H |
| 1267 | Cz3 | m-D3 | Cz3 | H | H |
| 1268 | Cz3 | Cz3 | m-D3 | H | H |
| 1269 | m-D3 | Cz3 | H | Cz3 | H |
| 1270 | Cz3 | m-D3 | H | Cz3 | H |
| 1271 | Cz3 | Cz3 | H | m-D3 | H |
| 1272 | m-D3 | H | Cz3 | Cz3 | H |
| 1273 | Cz3 | H | m-D3 | Cz3 | H |
| 1274 | Cz3 | H | Cz3 | m-D3 | H |
| 1275 | H | m-D3 | Cz3 | Cz3 | H |
| 1276 | H | Cz3 | m-D3 | Cz3 | H |
| 1277 | m-D3 | Cz3 | H | H | Cz3 |
| 1278 | Cz3 | m-D3 | H | H | Cz3 |
| 1279 | Cz3 | Cz3 | H | H | m-D3 |
| 1280 | m-D3 | H | Cz3 | H | Cz3 |
| 1281 | Cz3 | H | m-D3 | H | Cz3 |
| 1282 | m-D3 | Cz3 | H | H | H |
| 1283 | Cz3 | m-D3 | H | H | H |
| 1284 | m-D3 | H | Cz3 | H | H |
| 1285 | Cz3 | H | m-D3 | H | H |
| 1286 | H | m-D3 | Cz3 | H | H |
| 1287 | H | Cz3 | m-D3 | H | H |
| 1288 | m-D3 | H | H | Cz3 | H |
| 1289 | Cz3 | H | H | m-D3 | H |
| 1290 | H | m-D3 | H | Cz3 | H |
| 1291 | m-D3 | H | H | H | Cz3 |
| 1292 | m-D3 | m-D3 | Cz3 | Cz3 | H |
| 1293 | m-D3 | Cz3 | m-D3 | Cz3 | H |
| 1294 | m-D3 | Cz3 | Cz3 | m-D3 | H |
| 1295 | Cz3 | m-D3 | m-D3 | Cz3 | H |
| 1296 | Cz3 | m-D3 | Cz3 | m-D3 | H |
| 1297 | Cz3 | Cz3 | m-D3 | m-D3 | H |
| 1298 | m-D3 | m-D3 | Cz3 | H | Cz3 |
| 1299 | m-D3 | Cz3 | m-D3 | H | Cz3 |
| 1300 | m-D3 | Cz3 | Cz3 | H | m-D3 |
| 1301 | Cz3 | m-D3 | m-D3 | H | Cz3 |
| 1302 | Cz3 | m-D3 | Cz3 | H | m-D3 |
| 1303 | Cz3 | Cz3 | m-D3 | H | m-D3 |
| 1304 | m-D3 | m-D3 | H | Cz3 | Cz3 |
| 1305 | m-D3 | Cz3 | H | m-D3 | Cz3 |
| 1306 | m-D3 | Cz3 | H | Cz3 | m-D3 |
| 1307 | Cz3 | m-D3 | H | m-D3 | Cz3 |
| 1308 | m-D3 | m-D3 | Cz3 | H | H |
| 1309 | m-D3 | Cz3 | m-D3 | H | H |
| 1310 | Cz3 | m-D3 | m-D3 | H | H |
| 1311 | m-D3 | m-D3 | H | Cz3 | H |
| 1312 | m-D3 | Cz3 | H | m-D3 | H |
| 1313 | Cz3 | m-D3 | H | m-D3 | H |
| 1314 | m-D3 | H | m-D3 | Cz3 | H |
| 1315 | m-D3 | H | Cz3 | m-D3 | H |
| 1316 | Cz3 | H | m-D3 | m-D3 | H |
| 1317 | H | m-D3 | m-D3 | Cz3 | H |
| 1318 | H | m-D3 | Cz3 | m-D3 | H |
| 1319 | m-D3 | m-D3 | H | H | Cz3 |
| 1320 | m-D3 | Cz3 | H | H | m-D3 |
| 1321 | Cz3 | m-D3 | H | H | m-D3 |
| 1322 | m-D3 | H | m-D3 | H | Cz3 |
| 1323 | m-D3 | H | Cz3 | H | m-D3 |
| 1324 | m-D3 | m-D3 | m-D3 | Cz3 | Cz3 |
| 1325 | m-D3 | m-D3 | Cz3 | m-D3 | Cz3 |
| 1326 | m-D3 | m-D3 | Cz3 | Cz3 | m-D3 |
| 1327 | m-D3 | Cz3 | m-D3 | m-D3 | Cz3 |
| 1328 | m-D3 | Cz3 | m-D3 | Cz3 | m-D3 |
| 1329 | m-D3 | m-D3 | m-D3 | Cz3 | H |
| 1330 | m-D3 | m-D3 | Cz3 | m-D3 | H |
| 1331 | m-D3 | Cz3 | m-D3 | Cz3 | H |
| 1332 | Cz3 | m-D3 | m-D3 | m-D3 | H |
| 1333 | m-D3 | m-D3 | m-D3 | H | Cz3 |
| 1334 | m-D3 | m-D3 | Cz3 | H | m-D3 |
| 1335 | m-D3 | Cz3 | m-D3 | H | m-D3 |
| 1336 | Cz3 | m-D3 | m-D3 | H | m-D3 |
| 1337 | m-D3 | m-D3 | H | m-D3 | Cz3 |
| 1338 | m-D3 | m-D3 | H | Cz3 | m-D3 |
| 1339 | m-D3 | m-D3 | m-D3 | m-D3 | Cz3 |
| 1340 | m-D3 | m-D3 | m-D3 | Cz3 | m-D3 |
| 1341 | m-D3 | m-D3 | Cz3 | m-D3 | m-D3 |
| 1342 | Cz4 | Cz4 | m-D1 | Cz4 | Cz4 |
| 1343 | m-D1 | Cz4 | Cz4 | Cz4 | H |
| 1344 | Cz4 | Cz4 | Cz4 | m-D1 | H |
| 1345 | m-D1 | Cz4 | Cz4 | H | Cz4 |
| 1346 | Cz4 | Cz4 | m-D1 | H | Cz4 |
| 1347 | Cz4 | m-D1 | H | Cz4 | Cz4 |
| 1348 | Cz4 | m-D1 | Cz4 | H | H |
| 1349 | Cz4 | Cz4 | m-D1 | H | H |
| 1350 | H | m-D1 | Cz4 | Cz4 | H |
| 1351 | H | Cz4 | m-D1 | Cz4 | H |
| 1352 | m-D1 | H | Cz4 | H | Cz4 |
| 1353 | Cz4 | H | m-D1 | H | Cz4 |
| 1354 | m-D1 | Cz4 | H | H | H |
| 1355 | H | m-D1 | H | Cz4 | H |
| 1356 | m-D1 | m-D1 | Cz4 | H | Cz4 |
| 1357 | m-D1 | Cz4 | m-D1 | H | Cz4 |
| 1358 | Cz4 | m-D1 | m-D1 | H | Cz4 |
| 1359 | m-D1 | m-D1 | H | Cz4 | Cz4 |
| 1360 | Cz4 | m-D1 | H | m-D1 | Cz4 |
| 1361 | m-D1 | Cz4 | m-D1 | H | H |
| 1362 | H | m-D1 | m-D1 | Cz4 | H |
| 1363 | H | m-D1 | Cz4 | m-D1 | H |
| 1364 | m-D1 | H | m-D1 | H | Cz4 |
| 1365 | m-D1 | H | Cz4 | H | m-D1 |
| 1366 | m-D1 | Cz4 | m-D1 | Cz4 | m-D1 |
| 1367 | m-D1 | m-D1 | m-D1 | Cz4 | H |
| 1368 | m-D1 | m-D1 | Cz4 | m-D1 | H |
| 1369 | m-D1 | m-D1 | m-D1 | H | Cz4 |
| 1370 | m-D1 | m-D1 | H | m-D1 | Cz4 |
| 1371 | m-D1 | m-D1 | H | Cz4 | m-D1 |
| 1372 | m-D1 | m-D1 | m-D1 | m-D1 | Cz4 |
| 1373 | Cz4 | Cz4 | m-D2 | Cz4 | Cz4 |
| 1374 | m-D2 | Cz4 | Cz4 | Cz4 | H |
| 1375 | Cz4 | Cz4 | Cz4 | m-D2 | H |
| 1376 | m-D2 | Cz4 | Cz4 | H | Cz4 |
| 1377 | Cz4 | Cz4 | m-D2 | H | Cz4 |
| 1378 | Cz4 | m-D2 | H | Cz4 | Cz4 |
| 1379 | Cz4 | m-D2 | Cz4 | H | H |
| 1380 | Cz4 | Cz4 | m-D2 | H | H |
| 1381 | H | m-D2 | Cz4 | Cz4 | H |
| 1382 | H | Cz4 | m-D2 | Cz4 | H |
| 1383 | m-D2 | H | Cz4 | H | Cz4 |
| 1384 | Cz4 | H | m-D2 | H | Cz4 |
| 1385 | m-D2 | Cz4 | H | H | H |
| 1386 | H | m-D2 | H | Cz4 | H |
| 1387 | m-D2 | m-D2 | Cz4 | H | Cz4 |
| 1388 | m-D2 | Cz4 | m-D2 | H | Cz4 |
| 1389 | Cz4 | m-D2 | m-D2 | H | Cz4 |
| 1390 | m-D2 | m-D2 | H | Cz4 | Cz4 |
| 1391 | Cz4 | m-D2 | H | m-D2 | Cz4 |
| 1392 | m-D2 | Cz4 | m-D2 | H | H |
| 1393 | H | m-D2 | m-D2 | Cz4 | H |
| 1394 | H | m-D2 | Cz4 | m-D2 | H |
| 1395 | m-D2 | H | m-D2 | H | Cz4 |
| 1396 | m-D2 | H | Cz4 | H | m-D2 |
| 1397 | m-D2 | Cz4 | m-D2 | Cz4 | m-D2 |
| 1398 | m-D2 | m-D2 | m-D2 | Cz4 | H |
| 1399 | m-D2 | m-D2 | Cz4 | m-D2 | H |
| 1400 | m-D2 | m-D2 | m-D2 | H | Cz4 |
| 1401 | m-D2 | m-D2 | H | m-D2 | Cz4 |
| 1402 | m-D2 | m-D2 | H | Cz4 | m-D2 |
| 1403 | m-D2 | m-D2 | m-D2 | m-D2 | Cz4 |
| 1404 | Cz4 | Cz4 | m-D3 | Cz4 | Cz4 |
| 1405 | m-D3 | Cz4 | Cz4 | Cz4 | H |
| 1406 | Cz4 | Cz4 | Cz4 | m-D3 | H |
| 1407 | m-D3 | Cz4 | Cz4 | H | Cz4 |
| 1408 | Cz4 | Cz4 | m-D3 | H | Cz4 |
| 1409 | Cz4 | m-D3 | H | Cz4 | Cz4 |
| 1410 | Cz4 | m-D3 | Cz4 | H | H |
| 1411 | Cz4 | Cz4 | m-D3 | H | H |
| 1412 | H | m-D3 | Cz4 | Cz4 | H |
| 1413 | H | Cz4 | m-D3 | Cz4 | H |
| 1414 | m-D3 | H | Cz4 | H | Cz4 |
| 1415 | Cz4 | H | m-D3 | H | Cz4 |
| 1416 | m-D3 | Cz4 | H | H | H |
| 1417 | H | m-D3 | H | Cz4 | H |
| 1418 | m-D3 | m-D3 | Cz4 | H | Cz4 |
| 1419 | m-D3 | Cz4 | m-D3 | H | Cz4 |
| 1420 | Cz4 | m-D3 | m-D3 | H | Cz4 |
| 1421 | m-D3 | m-D3 | H | Cz4 | Cz4 |
| 1422 | Cz4 | m-D3 | H | m-D3 | Cz4 |
| 1423 | m-D3 | Cz4 | m-D3 | H | H |
| 1424 | H | m-D3 | m-D3 | Cz4 | H |
| 1425 | H | m-D3 | Cz4 | m-D3 | H |
| 1426 | m-D3 | H | m-D3 | H | Cz4 |
| 1427 | m-D3 | H | Cz4 | H | m-D3 |
| 1428 | m-D3 | Cz4 | m-D3 | Cz4 | m-D3 |
| 1429 | m-D3 | m-D3 | m-D3 | Cz4 | H |
| 1430 | m-D3 | m-D3 | Cz4 | m-D3 | H |
| 1431 | m-D3 | m-D3 | m-D3 | H | Cz4 |
| 1432 | m-D3 | m-D3 | H | m-D3 | Cz4 |
| 1433 | m-D3 | m-D3 | H | Cz4 | m-D3 |
| 1434 | m-D3 | m-D3 | m-D3 | m-D3 | Cz4 |
| 1435 | Cz5 | Cz5 | m-D1 | Cz5 | Cz5 |
| 1436 | m-D1 | Cz5 | Cz5 | Cz5 | H |
| 1437 | Cz5 | Cz5 | Cz5 | m-D1 | H |
| 1438 | m-D1 | Cz5 | Cz5 | H | Cz5 |
| 1439 | Cz5 | Cz5 | m-D1 | H | Cz5 |
| 1440 | Cz5 | m-D1 | H | Cz5 | Cz5 |
| 1441 | Cz5 | m-D1 | Cz5 | H | H |
| 1442 | Cz5 | Cz5 | m-D1 | H | H |
| 1443 | H | m-D1 | Cz5 | Cz5 | H |
| 1444 | H | Cz5 | m-D1 | Cz5 | H |
| 1445 | m-D1 | H | Cz5 | H | Cz5 |
| 1446 | Cz5 | H | m-D1 | H | Cz5 |
| 1447 | m-D1 | Cz5 | H | H | H |
| 1448 | H | m-D1 | H | Cz5 | H |
| 1449 | m-D1 | m-D1 | Cz5 | H | Cz5 |
| 1450 | m-D1 | Cz5 | m-D1 | H | Cz5 |
| 1451 | Cz5 | m-D1 | m-D1 | H | Cz5 |
| 1452 | m-D1 | m-D1 | H | Cz5 | Cz5 |
| 1453 | Cz5 | m-D1 | H | m-D1 | Cz5 |
| 1454 | m-D1 | Cz5 | m-D1 | H | H |
| 1455 | H | m-D1 | m-D1 | Cz5 | H |
| 1456 | H | m-D1 | Cz5 | m-D1 | H |
| 1457 | m-D1 | H | m-D1 | H | Cz5 |
| 1458 | m-D1 | H | Cz5 | H | m-D1 |
| 1459 | m-D1 | Cz5 | m-D1 | Cz5 | m-D1 |
| 1460 | m-D1 | m-D1 | m-D1 | Cz5 | H |
| 1461 | m-D1 | m-D1 | Cz5 | m-D1 | H |
| 1462 | m-D1 | m-D1 | m-D1 | H | Cz5 |
| 1463 | m-D1 | m-D1 | H | m-D1 | Cz5 |
| 1464 | m-D1 | m-D1 | H | Cz5 | m-D1 |
| 1465 | m-D1 | m-D1 | m-D1 | m-D1 | Cz5 |
| 1466 | Cz5 | Cz5 | m-D2 | Cz5 | Cz5 |
| 1467 | m-D2 | Cz5 | Cz5 | Cz5 | H |
| 1468 | Cz5 | Cz5 | Cz5 | m-D2 | H |
| 1469 | m-D2 | Cz5 | Cz5 | H | Cz5 |
| 1470 | Cz5 | Cz5 | m-D2 | H | Cz5 |
| 1471 | Cz5 | m-D2 | H | Cz5 | Cz5 |
| 1472 | Cz5 | m-D2 | Cz5 | H | H |
| 1473 | Cz5 | Cz5 | m-D2 | H | H |
| 1474 | H | m-D2 | Cz5 | Cz5 | H |
| 1475 | H | Cz5 | m-D2 | Cz5 | H |
| 1476 | m-D2 | H | Cz5 | H | Cz5 |
| 1477 | Cz5 | H | m-D2 | H | Cz5 |
| 1478 | m-D2 | Cz5 | H | H | H |
| 1479 | H | m-D2 | H | Cz5 | H |
| 1480 | m-D2 | m-D2 | Cz5 | H | Cz5 |
| 1481 | m-D2 | Cz5 | m-D2 | H | Cz5 |
| 1482 | Cz5 | m-D2 | m-D2 | H | Cz5 |
| 1483 | m-D2 | m-D2 | H | Cz5 | Cz5 |
| 1484 | Cz5 | m-D2 | H | m-D2 | Cz5 |
| 1485 | m-D2 | Cz5 | m-D2 | H | H |
| 1486 | H | m-D2 | m-D2 | Cz5 | H |
| 1487 | H | m-D2 | Cz5 | m-D2 | H |
| 1488 | m-D2 | H | m-D2 | H | Cz5 |
| 1489 | m-D2 | H | Cz5 | H | m-D2 |
| 1490 | m-D2 | Cz5 | m-D2 | Cz5 | m-D2 |
| 1491 | m-D2 | m-D2 | m-D2 | Cz5 | H |
| 1492 | m-D2 | m-D2 | Cz5 | m-D2 | H |
| 1493 | m-D2 | m-D2 | m-D2 | H | Cz5 |
| 1494 | m-D2 | m-D2 | H | m-D2 | Cz5 |
| 1495 | m-D2 | m-D2 | H | Cz5 | m-D2 |
| 1496 | m-D2 | m-D2 | m-D2 | m-D2 | Cz5 |
| 1497 | Cz5 | Cz5 | m-D3 | Cz5 | Cz5 |
| 1498 | m-D3 | Cz5 | Cz5 | Cz5 | H |
| 1499 | Cz5 | Cz5 | Cz5 | m-D3 | H |
| 1500 | m-D3 | Cz5 | Cz5 | H | Cz5 |
| 1501 | Cz5 | Cz5 | m-D3 | H | Cz5 |
| 1502 | Cz5 | m-D3 | H | Cz5 | Cz5 |
| 1503 | Cz5 | m-D3 | Cz5 | H | H |
| 1504 | Cz5 | Cz5 | m-D3 | H | H |
| 1505 | H | m-D3 | Cz5 | Cz5 | H |
| 1506 | H | Cz5 | m-D3 | Cz5 | H |
| 1507 | m-D3 | H | Cz5 | H | Cz5 |
| 1508 | Cz5 | H | m-D3 | H | Cz5 |
| 1509 | m-D3 | Cz5 | H | H | H |
| 1510 | H | m-D3 | H | Cz5 | H |
| 1511 | m-D3 | m-D3 | Cz5 | H | Cz5 |
| 1512 | m-D3 | Cz5 | m-D3 | H | Cz5 |
| 1513 | Cz5 | m-D3 | m-D3 | H | Cz5 |
| 1514 | m-D3 | m-D3 | H | Cz5 | Cz5 |
| 1515 | Cz5 | m-D3 | H | m-D3 | Cz5 |
| 1516 | m-D3 | Cz5 | m-D3 | H | H |
| 1517 | H | m-D3 | m-D3 | Cz5 | H |
| 1518 | H | m-D3 | Cz5 | m-D3 | H |
| 1519 | m-D3 | H | m-D3 | H | Cz5 |
| 1520 | m-D3 | H | Cz5 | H | m-D3 |
| 1521 | m-D3 | Cz5 | m-D3 | Cz5 | m-D3 |
| 1522 | m-D3 | m-D3 | m-D3 | Cz5 | H |
| 1523 | m-D3 | m-D3 | Cz5 | m-D3 | H |
| 1524 | m-D3 | m-D3 | m-D3 | H | Cz5 |
| 1525 | m-D3 | m-D3 | H | m-D3 | Cz5 |
| 1526 | m-D3 | m-D3 | H | Cz5 | m-D3 |
| 1527 | m-D3 | m-D3 | m-D3 | m-D3 | Cz5 |
| 1528 | Cz6 | Cz6 | m-D1 | Cz6 | Cz6 |
| 1529 | m-D1 | Cz6 | Cz6 | Cz6 | H |
| 1530 | Cz6 | Cz6 | Cz6 | m-D1 | H |
| 1531 | m-D1 | Cz6 | Cz6 | H | Cz6 |
| 1532 | Cz6 | Cz6 | m-D1 | H | Cz6 |
| 1533 | Cz6 | m-D1 | H | Cz6 | Cz6 |
| 1534 | Cz6 | m-D1 | Cz6 | H | H |
| 1535 | Cz6 | Cz6 | m-D1 | H | H |
| 1536 | H | m-D1 | Cz6 | Cz6 | H |
| 1537 | H | Cz6 | m-D1 | Cz6 | H |
| 1538 | m-D1 | H | Cz6 | H | Cz6 |
| 1539 | Cz6 | H | m-D1 | H | Cz6 |
| 1540 | m-D1 | Cz6 | H | H | H |
| 1541 | H | m-D1 | H | Cz6 | H |
| 1542 | m-D1 | m-D1 | Cz6 | H | Cz6 |
| 1543 | m-D1 | Cz6 | m-D1 | H | Cz6 |
| 1544 | Cz6 | m-D1 | m-D1 | H | Cz6 |
| 1545 | m-D1 | m-D1 | H | Cz6 | Cz6 |
| 1546 | Cz6 | m-D1 | H | m-D1 | Cz6 |
| 1547 | m-D1 | Cz6 | m-D1 | H | H |
| 1548 | H | m-D1 | m-D1 | Cz6 | H |
| 1549 | H | m-D1 | Cz6 | m-D1 | H |
| 1550 | m-D1 | H | m-D1 | H | Cz6 |
| 1551 | m-D1 | H | Cz6 | H | m-D1 |
| 1552 | m-D1 | Cz6 | m-D1 | Cz6 | m-D1 |
| 1553 | m-D1 | m-D1 | m-D1 | Cz6 | H |
| 1554 | m-D1 | m-D1 | Cz6 | m-D1 | H |
| 1555 | m-D1 | m-D1 | m-D1 | H | Cz6 |
| 1556 | m-D1 | m-D1 | H | m-D1 | Cz6 |
| 1557 | m-D1 | m-D1 | H | Cz6 | m-D1 |
| 1558 | m-D1 | m-D1 | m-D1 | m-D1 | Cz6 |
| 1559 | Cz6 | Cz6 | m-D2 | Cz6 | Cz6 |
| 1560 | m-D2 | Cz6 | Cz6 | Cz6 | H |
| 1561 | Cz6 | Cz6 | Cz6 | m-D2 | H |
| 1562 | m-D2 | Cz6 | Cz6 | H | Cz6 |
| 1563 | Cz6 | Cz6 | m-D2 | H | Cz6 |
| 1564 | Cz6 | m-D2 | H | Cz6 | Cz6 |
| 1565 | Cz6 | m-D2 | Cz6 | H | H |
| 1566 | Cz6 | Cz6 | m-D2 | H | H |
| 1567 | H | m-D2 | Cz6 | Cz6 | H |
| 1568 | H | Cz6 | m-D2 | Cz6 | H |
| 1569 | m-D2 | H | Cz6 | H | Cz6 |
| 1570 | Cz6 | H | m-D2 | H | Cz6 |
| 1571 | m-D2 | Cz6 | H | H | H |
| 1572 | H | m-D2 | H | Cz6 | H |
| 1573 | m-D2 | m-D2 | Cz6 | H | Cz6 |
| 1574 | m-D2 | Cz6 | m-D2 | H | Cz6 |
| 1575 | Cz6 | m-D2 | m-D2 | H | Cz6 |
| 1576 | m-D2 | m-D2 | H | Cz6 | Cz6 |
| 1577 | Cz6 | m-D2 | H | m-D2 | Cz6 |
| 1578 | m-D2 | Cz6 | m-D2 | H | H |
| 1579 | H | m-D2 | m-D2 | Cz6 | H |
| 1580 | H | m-D2 | Cz6 | m-D2 | H |
| 1581 | m-D2 | H | m-D2 | H | Cz6 |
| 1582 | m-D2 | H | Cz6 | H | m-D2 |
| 1583 | m-D2 | Cz6 | m-D2 | Cz6 | m-D2 |
| 1584 | m-D2 | m-D2 | m-D2 | Cz6 | H |
| 1585 | m-D2 | m-D2 | Cz6 | m-D2 | H |
| 1586 | m-D2 | m-D2 | m-D2 | H | Cz6 |
| 1587 | m-D2 | m-D2 | H | m-D2 | Cz6 |
| 1588 | m-D2 | m-D2 | H | Cz6 | m-D2 |
| 1589 | m-D2 | m-D2 | m-D2 | m-D2 | Cz6 |
| 1590 | Cz6 | Cz6 | m-D3 | Cz6 | Cz6 |
| 1591 | m-D3 | Cz6 | Cz6 | Cz6 | H |
| 1592 | Cz6 | Cz6 | Cz6 | m-D3 | H |
| 1593 | m-D3 | Cz6 | Cz6 | H | Cz6 |
| 1594 | Cz6 | Cz6 | m-D3 | H | Cz6 |
| 1595 | Cz6 | m-D3 | H | Cz6 | Cz6 |
| 1596 | Cz6 | m-D3 | Cz6 | H | H |
| 1597 | Cz6 | Cz6 | m-D3 | H | H |
| 1598 | H | m-D3 | Cz6 | Cz6 | H |
| 1599 | H | Cz6 | m-D3 | Cz6 | H |
| 1600 | m-D3 | H | Cz6 | H | Cz6 |
| 1601 | Cz6 | H | m-D3 | H | Cz6 |
| 1602 | m-D3 | Cz6 | H | H | H |
| 1603 | H | m-D3 | H | Cz6 | H |
| 1604 | m-D3 | m-D3 | Cz6 | H | Cz6 |
| 1605 | m-D3 | Cz6 | m-D3 | H | Cz6 |
| 1606 | Cz6 | m-D3 | m-D3 | H | Cz6 |
| 1607 | m-D3 | m-D3 | H | Cz6 | Cz6 |
| 1608 | Cz6 | m-D3 | H | m-D3 | Cz6 |
| 1609 | m-D3 | Cz6 | m-D3 | H | H |
| 1610 | H | m-D3 | m-D3 | Cz6 | H |
| 1611 | H | m-D3 | Cz6 | m-D3 | H |
| 1612 | m-D3 | H | m-D3 | H | Cz6 |
| 1613 | m-D3 | H | Cz6 | H | m-D3 |
| 1614 | m-D3 | Cz6 | m-D3 | Cz6 | m-D3 |
| 1615 | m-D3 | m-D3 | m-D3 | Cz6 | H |
| 1616 | m-D3 | m-D3 | Cz6 | m-D3 | H |
| 1617 | m-D3 | m-D3 | m-D3 | H | Cz6 |
| 1618 | m-D3 | m-D3 | H | m-D3 | Cz6 |
| 1619 | m-D3 | m-D3 | H | Cz6 | m-D3 |
| 1620 | m-D3 | m-D3 | m-D3 | m-D3 | Cz6 |
| 1621 | Cz7 | Cz7 | m-D1 | Cz7 | Cz7 |
| 1622 | m-D1 | Cz7 | Cz7 | Cz7 | H |
| 1623 | Cz7 | Cz7 | Cz7 | m-D1 | H |
| 1624 | m-D1 | Cz7 | Cz7 | H | Cz7 |
| 1625 | Cz7 | Cz7 | m-D1 | H | Cz7 |
| 1626 | Cz7 | m-D1 | H | Cz7 | Cz7 |
| 1627 | Cz7 | m-D1 | Cz7 | H | H |
| 1628 | Cz7 | Cz7 | m-D1 | H | H |
| 1629 | H | m-D1 | Cz7 | Cz7 | H |
| 1630 | H | Cz7 | m-D1 | Cz7 | H |
| 1631 | m-D1 | H | Cz7 | H | Cz7 |
| 1632 | Cz7 | H | m-D1 | H | Cz7 |
| 1633 | m-D1 | Cz7 | H | H | H |
| 1634 | H | m-D1 | H | Cz7 | H |
| 1635 | m-D1 | m-D1 | Cz7 | H | Cz7 |
| 1636 | m-D1 | Cz7 | m-D1 | H | Cz7 |
| 1637 | Cz7 | m-D1 | m-D1 | H | Cz7 |
| 1638 | m-D1 | m-D1 | H | Cz7 | Cz7 |
| 1639 | Cz7 | m-D1 | H | m-D1 | Cz7 |
| 1640 | m-D1 | Cz7 | m-D1 | H | H |
| 1641 | H | m-D1 | m-D1 | Cz7 | H |
| 1642 | H | m-D1 | Cz7 | m-D1 | H |
| 1643 | m-D1 | H | m-D1 | H | Cz7 |
| 1644 | m-D1 | H | Cz7 | H | m-D1 |
| 1645 | m-D1 | Cz7 | m-D1 | Cz7 | m-D1 |
| 1646 | m-D1 | m-D1 | m-D1 | Cz7 | H |
| 1647 | m-D1 | m-D1 | Cz7 | m-D1 | H |
| 1648 | m-D1 | m-D1 | m-D1 | H | Cz7 |
| 1649 | m-D1 | m-D1 | H | m-D1 | Cz7 |
| 1650 | m-D1 | m-D1 | H | Cz7 | m-D1 |
| 1651 | m-D1 | m-D1 | m-D1 | m-D1 | Cz7 |
| 1652 | Cz7 | Cz7 | m-D2 | Cz7 | Cz7 |
| 1653 | m-D2 | Cz7 | Cz7 | Cz7 | H |
| 1654 | Cz7 | Cz7 | Cz7 | m-D2 | H |
| 1655 | m-D2 | Cz7 | Cz7 | H | Cz7 |
| 1656 | Cz7 | Cz7 | m-D2 | H | Cz7 |
| 1657 | Cz7 | m-D2 | H | Cz7 | Cz7 |
| 1658 | Cz7 | m-D2 | Cz7 | H | H |
| 1659 | Cz7 | Cz7 | m-D2 | H | H |
| 1660 | H | m-D2 | Cz7 | Cz7 | H |
| 1661 | H | Cz7 | m-D2 | Cz7 | H |
| 1662 | m-D2 | H | Cz7 | H | Cz7 |
| 1663 | Cz7 | H | m-D2 | H | Cz7 |
| 1664 | m-D2 | Cz7 | H | H | H |
| 1665 | H | m-D2 | H | Cz7 | H |
| 1666 | m-D2 | m-D2 | Cz7 | H | Cz7 |
| 1667 | m-D2 | Cz7 | m-D2 | H | Cz7 |
| 1668 | Cz7 | m-D2 | m-D2 | H | Cz7 |
| 1669 | m-D2 | m-D2 | H | Cz7 | Cz7 |
| 1670 | Cz7 | m-D2 | H | m-D2 | Cz7 |
| 1671 | m-D2 | Cz7 | m-D2 | H | H |
| 1672 | H | m-D2 | m-D2 | Cz7 | H |
| 1673 | H | m-D2 | Cz7 | m-D2 | H |
| 1674 | m-D2 | H | m-D2 | H | Cz7 |
| 1675 | m-D2 | H | Cz7 | H | m-D2 |
| 1676 | m-D2 | Cz7 | m-D2 | Cz7 | m-D2 |
| 1677 | m-D2 | m-D2 | m-D2 | Cz7 | H |
| 1678 | m-D2 | m-D2 | Cz7 | m-D2 | H |
| 1679 | m-D2 | m-D2 | m-D2 | H | Cz7 |
| 1680 | m-D2 | m-D2 | H | m-D2 | Cz7 |
| 1681 | m-D2 | m-D2 | H | Cz7 | m-D2 |
| 1682 | m-D2 | m-D2 | m-D2 | m-D2 | Cz7 |
| 1683 | Cz7 | Cz7 | m-D3 | Cz7 | Cz7 |
| 1684 | m-D3 | Cz7 | Cz7 | Cz7 | H |
| 1685 | Cz7 | Cz7 | Cz7 | m-D3 | H |
| 1686 | m-D3 | Cz7 | Cz7 | H | Cz7 |
| 1687 | Cz7 | Cz7 | m-D3 | H | Cz7 |
| 1688 | Cz7 | m-D3 | H | Cz7 | Cz7 |
| 1689 | Cz7 | m-D3 | Cz7 | H | H |
| 1690 | Cz7 | Cz7 | m-D3 | H | H |
| 1691 | H | m-D3 | Cz7 | Cz7 | H |
| 1692 | H | Cz7 | m-D3 | Cz7 | H |
| 1693 | m-D3 | H | Cz7 | H | Cz7 |
| 1694 | Cz7 | H | m-D3 | H | Cz7 |
| 1695 | m-D3 | Cz7 | H | H | H |
| 1696 | H | m-D3 | H | Cz7 | H |
| 1697 | m-D3 | m-D3 | Cz7 | H | Cz7 |
| 1698 | m-D3 | Cz7 | m-D3 | H | Cz7 |
| 1699 | Cz7 | m-D3 | m-D3 | H | Cz7 |
| 1700 | m-D3 | m-D3 | H | Cz7 | Cz7 |
| 1701 | Cz7 | m-D3 | H | m-D3 | Cz7 |
| 1702 | m-D3 | Cz7 | m-D3 | H | H |
| 1703 | H | m-D3 | m-D3 | Cz7 | H |
| 1704 | H | m-D3 | Cz7 | m-D3 | H |
| 1705 | m-D3 | H | m-D3 | H | Cz7 |
| 1706 | m-D3 | H | Cz7 | H | m-D3 |
| 1707 | m-D3 | Cz7 | m-D3 | Cz7 | m-D3 |
| 1708 | m-D3 | m-D3 | m-D3 | Cz7 | H |
| 1709 | m-D3 | m-D3 | Cz7 | m-D3 | H |
| 1710 | m-D3 | m-D3 | m-D3 | H | Cz7 |
| 1711 | m-D3 | m-D3 | H | m-D3 | Cz7 |
| 1712 | m-D3 | m-D3 | H | Cz7 | m-D3 |
| 1713 | m-D3 | m-D3 | m-D3 | m-D3 | Cz7 |
| 1714 | Cz8 | Cz8 | m-D1 | Cz8 | Cz8 |
| 1715 | m-D1 | Cz8 | Cz8 | Cz8 | H |
| 1716 | Cz8 | Cz8 | Cz8 | m-D1 | H |
| 1717 | m-D1 | Cz8 | Cz8 | H | Cz8 |
| 1718 | Cz8 | Cz8 | m-D1 | H | Cz8 |
| 1719 | Cz8 | m-D1 | H | Cz8 | Cz8 |
| 1720 | Cz8 | m-D1 | Cz8 | H | H |
| 1721 | Cz8 | Cz8 | m-D1 | H | H |
| 1722 | H | m-D1 | Cz8 | Cz8 | H |
| 1723 | H | Cz8 | m-D1 | Cz8 | H |
| 1724 | m-D1 | H | Cz8 | H | Cz8 |
| 1725 | Cz8 | H | m-D1 | H | Cz8 |
| 1726 | m-D1 | Cz8 | H | H | H |
| 1727 | H | m-D1 | H | Cz8 | H |
| 1728 | m-D1 | m-D1 | Cz8 | H | Cz8 |
| 1729 | m-D1 | Cz8 | m-D1 | H | Cz8 |
| 1730 | Cz8 | m-D1 | m-D1 | H | Cz8 |
| 1731 | m-D1 | m-D1 | H | Cz8 | Cz8 |
| 1732 | Cz8 | m-D1 | H | m-D1 | Cz8 |
| 1733 | m-D1 | Cz8 | m-D1 | H | H |
| 1734 | H | m-D1 | m-D1 | Cz8 | H |
| 1735 | H | m-D1 | Cz8 | m-D1 | H |
| 1736 | m-D1 | H | m-D1 | H | Cz8 |
| 1737 | m-D1 | H | Cz8 | H | m-D1 |
| 1738 | m-D1 | Cz8 | m-D1 | Cz8 | m-D1 |
| 1739 | m-D1 | m-D1 | m-D1 | Cz8 | H |
| 1740 | m-D1 | m-D1 | Cz8 | m-D1 | H |
| 1741 | m-D1 | m-D1 | m-D1 | H | Cz8 |
| 1742 | m-D1 | m-D1 | H | m-D1 | Cz8 |
| 1743 | m-D1 | m-D1 | H | Cz8 | m-D1 |
| 1744 | m-D1 | m-D1 | m-D1 | m-D1 | Cz8 |
| 1745 | Cz8 | Cz8 | m-D2 | Cz8 | Cz8 |
| 1746 | m-D2 | Cz8 | Cz8 | Cz8 | H |
| 1747 | Cz8 | Cz8 | Cz8 | m-D2 | H |
| 1748 | m-D2 | Cz8 | Cz8 | H | Cz8 |
| 1749 | Cz8 | Cz8 | m-D2 | H | Cz8 |
| 1750 | Cz8 | m-D2 | H | Cz8 | Cz8 |
| 1751 | Cz8 | m-D2 | Cz8 | H | H |
| 1752 | Cz8 | Cz8 | m-D2 | H | H |
| 1753 | H | m-D2 | Cz8 | Cz8 | H |
| 1754 | H | Cz8 | m-D2 | Cz8 | H |
| 1755 | m-D2 | H | Cz8 | H | Cz8 |
| 1756 | Cz8 | H | m-D2 | H | Cz8 |
| 1757 | m-D2 | Cz8 | H | H | H |
| 1758 | H | m-D2 | H | Cz8 | H |
| 1759 | m-D2 | m-D2 | Cz8 | H | Cz8 |
| 1760 | m-D2 | Cz8 | m-D2 | H | Cz8 |
| 1761 | Cz8 | m-D2 | m-D2 | H | Cz8 |
| 1762 | m-D2 | m-D2 | H | Cz8 | Cz8 |
| 1763 | Cz8 | m-D2 | H | m-D2 | Cz8 |
| 1764 | m-D2 | Cz8 | m-D2 | H | H |
| 1765 | H | m-D2 | m-D2 | Cz8 | H |
| 1766 | H | m-D2 | Cz8 | m-D2 | H |
| 1767 | m-D2 | H | m-D2 | H | Cz8 |
| 1768 | m-D2 | H | Cz8 | H | m-D2 |
| 1769 | m-D2 | Cz8 | m-D2 | Cz8 | m-D2 |
| 1770 | m-D2 | m-D2 | m-D2 | Cz8 | H |
| 1771 | m-D2 | m-D2 | Cz8 | m-D2 | H |
| 1772 | m-D2 | m-D2 | m-D2 | H | Cz8 |
| 1773 | m-D2 | m-D2 | H | m-D2 | Cz8 |
| 1774 | m-D2 | m-D2 | H | Cz8 | m-D2 |
| 1775 | m-D2 | m-D2 | m-D2 | m-D2 | Cz8 |
| 1776 | Cz8 | Cz8 | m-D3 | Cz8 | Cz8 |
| 1777 | m-D3 | Cz8 | Cz8 | Cz8 | H |
| 1778 | Cz8 | Cz8 | Cz8 | m-D3 | H |
| 1779 | m-D3 | Cz8 | Cz8 | H | Cz8 |
| 1780 | Cz8 | Cz8 | m-D3 | H | Cz8 |
| 1781 | Cz8 | m-D3 | H | Cz8 | Cz8 |
| 1782 | Cz8 | m-D3 | Cz8 | H | H |
| 1783 | Cz8 | Cz8 | m-D3 | H | H |
| 1784 | H | m-D3 | Cz8 | Cz8 | H |
| 1785 | H | Cz8 | m-D3 | Cz8 | H |
| 1786 | m-D3 | H | Cz8 | H | Cz8 |
| 1787 | Cz8 | H | m-D3 | H | Cz8 |
| 1788 | m-D3 | Cz8 | H | H | H |
| 1789 | H | m-D3 | H | Cz8 | H |
| 1790 | m-D3 | m-D3 | Cz8 | H | Cz8 |
| 1791 | m-D3 | Cz8 | m-D3 | H | Cz8 |
| 1792 | Cz8 | m-D3 | m-D3 | H | Cz8 |
| 1793 | m-D3 | m-D3 | H | Cz8 | Cz8 |
| 1794 | Cz8 | m-D3 | H | m-D3 | Cz8 |
| 1795 | m-D3 | Cz8 | m-D3 | H | H |
| 1796 | H | m-D3 | m-D3 | Cz8 | H |
| 1797 | H | m-D3 | Cz8 | m-D3 | H |
| 1798 | m-D3 | H | m-D3 | H | Cz8 |
| 1799 | m-D3 | H | Cz8 | H | m-D3 |
| 1800 | m-D3 | Cz8 | m-D3 | Cz8 | m-D3 |
| 1801 | m-D3 | m-D3 | m-D3 | Cz8 | H |
| 1802 | m-D3 | m-D3 | Cz8 | m-D3 | H |
| 1803 | m-D3 | m-D3 | m-D3 | H | Cz8 |
| 1804 | m-D3 | m-D3 | H | m-D3 | Cz8 |
| 1805 | m-D3 | m-D3 | H | Cz8 | m-D3 |
| 1806 | m-D3 | m-D3 | m-D3 | m-D3 | Cz8 |
| 1807 | m-D1 | Cz | Cz | Cz | methyl |
| 1808 | Cz | Cz | Cz | m-D1 | methyl |
| 1809 | m-D1 | Cz | Cz | methyl | Cz |
| 1810 | Cz | Cz | m-D1 | methyl | Cz |
| 1811 | Cz | m-D1 | methyl | Cz | Cz |
| 1812 | Cz | m-D1 | Cz | methyl | methyl |
| 1813 | Cz | Cz | m-D1 | methyl | methyl |
| 1814 | methyl | m-D1 | Cz | Cz | methyl |
| 1815 | methyl | Cz | m-D1 | Cz | methyl |
| 1816 | m-D1 | methyl | Cz | methyl | Cz |
| 1817 | Cz | methyl | m-D1 | methyl | Cz |
| 1818 | m-D1 | Cz | methyl | methyl | methyl |
| 1819 | methyl | m-D1 | methyl | Cz | methyl |
| 1820 | m-D1 | m-D1 | Cz | methyl | Cz |
| 1821 | m-D1 | Cz | m-D1 | methyl | Cz |
| 1822 | Cz | m-D1 | m-D1 | methyl | Cz |
| 1823 | m-D1 | m-D1 | methyl | Cz | Cz |
| 1824 | Cz | m-D1 | methyl | m-D1 | Cz |
| 1825 | m-D1 | Cz | m-D1 | methyl | methyl |
| 1826 | methyl | m-D1 | m-D1 | Cz | methyl |
| 1827 | methyl | m-D1 | Cz | m-D1 | methyl |
| 1828 | m-D1 | methyl | m-D1 | methyl | Cz |
| 1829 | m-D1 | methyl | Cz | methyl | m-D1 |
| 1830 | methyl | m-D1 | m-D1 | methyl | methyl |
| 1831 | m-D1 | m-D1 | m-D1 | Cz | methyl |
| 1832 | m-D1 | m-D1 | Cz | m-D1 | methyl |
| 1833 | m-D1 | m-D1 | m-D1 | methyl | Cz |
| 1834 | m-D1 | m-D1 | methyl | m-D1 | Cz |
| 1835 | m-D1 | m-D1 | methyl | Cz | m-D1 |
| 1836 | m-D1 | m-D1 | methyl | m-D1 | methyl |
| 1837 | methyl | m-D1 | m-D1 | m-D1 | methyl |
| 1838 | m-D1 | m-D1 | m-D1 | m-D1 | methyl |
| 1839 | m-D1 | m-D1 | methyl | m-D1 | m-D1 |
| 1840 | m-D2 | Cz | Cz | Cz | methyl |
| 1841 | Cz | Cz | Cz | m-D2 | methyl |
| 1842 | m-D2 | Cz | Cz | methyl | Cz |
| 1843 | Cz | Cz | m-D2 | methyl | Cz |
| 1844 | Cz | m-D2 | methyl | Cz | Cz |
| 1845 | Cz | m-D2 | Cz | methyl | methyl |
| 1846 | Cz | Cz | m-D2 | methyl | methyl |
| 1847 | methyl | m-D2 | Cz | Cz | methyl |
| 1848 | methyl | Cz | m-D2 | Cz | methyl |
| 1849 | m-D2 | methyl | Cz | methyl | Cz |
| 1850 | Cz | methyl | m-D2 | methyl | Cz |
| 1851 | m-D2 | Cz | methyl | methyl | methyl |
| 1852 | methyl | m-D2 | methyl | Cz | methyl |
| 1853 | m-D2 | m-D2 | Cz | methyl | Cz |
| 1854 | m-D2 | Cz | m-D2 | methyl | Cz |
| 1855 | Cz | m-D2 | m-D2 | methyl | Cz |
| 1856 | m-D2 | m-D2 | methyl | Cz | Cz |
| 1857 | Cz | m-D2 | methyl | m-D2 | Cz |
| 1858 | m-D2 | Cz | m-D2 | methyl | methyl |
| 1859 | methyl | m-D2 | m-D2 | Cz | methyl |
| 1860 | methyl | m-D2 | Cz | m-D2 | methyl |
| 1861 | m-D2 | methyl | m-D2 | methyl | Cz |
| 1862 | m-D2 | methyl | Cz | methyl | m-D2 |
| 1863 | methyl | m-D2 | m-D2 | methyl | methyl |
| 1864 | m-D2 | m-D2 | m-D2 | Cz | methyl |
| 1865 | m-D2 | m-D2 | Cz | m-D2 | methyl |
| 1866 | m-D2 | m-D2 | m-D2 | methyl | Cz |
| 1867 | m-D2 | m-D2 | methyl | m-D2 | Cz |
| 1868 | m-D2 | m-D2 | methyl | Cz | m-D2 |
| 1869 | m-D2 | m-D2 | methyl | m-D2 | methyl |
| 1870 | methyl | m-D2 | m-D2 | m-D2 | methyl |
| 1871 | m-D2 | m-D2 | m-D2 | m-D2 | methyl |
| 1872 | m-D2 | m-D2 | methyl | m-D2 | m-D2 |
| 1873 | m-D3 | Cz | Cz | Cz | methyl |
| 1874 | Cz | Cz | Cz | m-D3 | methyl |
| 1875 | m-D3 | Cz | Cz | methyl | Cz |
| 1876 | Cz | Cz | m-D3 | methyl | Cz |
| 1877 | Cz | m-D3 | methyl | Cz | Cz |
| 1878 | Cz | m-D3 | Cz | methyl | methyl |
| 1879 | Cz | Cz | m-D3 | methyl | methyl |
| 1880 | methyl | m-D3 | Cz | Cz | methyl |
| 1881 | methyl | Cz | m-D3 | Cz | methyl |
| 1882 | m-D3 | methyl | Cz | methyl | Cz |
| 1883 | Cz | methyl | m-D3 | methyl | Cz |
| 1884 | m-D3 | Cz | methyl | methyl | methyl |
| 1885 | methyl | m-D3 | methyl | Cz | methyl |
| 1886 | m-D3 | m-D3 | Cz | methyl | Cz |
| 1887 | m-D3 | Cz | m-D3 | methyl | Cz |
| 1888 | Cz | m-D3 | m-D3 | methyl | Cz |
| 1889 | m-D3 | m-D3 | methyl | Cz | Cz |
| 1890 | Cz | m-D3 | methyl | m-D3 | Cz |
| 1891 | m-D3 | Cz | m-D3 | methyl | methyl |
| 1892 | methyl | m-D3 | m-D3 | Cz | methyl |
| 1893 | methyl | m-D3 | Cz | m-D3 | methyl |
| 1894 | m-D3 | methyl | m-D3 | methyl | Cz |
| 1895 | m-D3 | methyl | Cz | methyl | m-D3 |
| 1896 | methyl | m-D3 | m-D3 | methyl | methyl |
| 1897 | m-D3 | m-D3 | m-D3 | Cz | methyl |
| 1898 | m-D3 | m-D3 | Cz | m-D3 | methyl |
| 1899 | m-D3 | m-D3 | m-D3 | methyl | Cz |
| 1900 | m-D3 | m-D3 | methyl | m-D3 | Cz |
| 1901 | m-D3 | m-D3 | methyl | Cz | m-D3 |
| 1902 | m-D3 | m-D3 | methyl | m-D3 | methyl |
| 1903 | methyl | m-D3 | m-D3 | m-D3 | methyl |
| 1904 | m-D3 | m-D3 | m-D3 | m-D3 | methyl |
| 1905 | m-D3 | m-D3 | methyl | m-D3 | m-D3 |
| 1906 | m-D1 | Cz | Cz | Cz | phenyl |
| 1907 | Cz | Cz | Cz | m-D1 | phenyl |
| 1908 | m-D1 | Cz | Cz | phenyl | Cz |
| 1909 | Cz | Cz | m-D1 | phenyl | Cz |
| 1910 | Cz | m-D1 | phenyl | Cz | Cz |
| 1911 | Cz | m-D1 | Cz | phenyl | phenyl |
| 1912 | Cz | Cz | m-D1 | phenyl | phenyl |
| 1913 | phenyl | m-D1 | Cz | Cz | phenyl |
| 1914 | phenyl | Cz | m-D1 | Cz | phenyl |
| 1915 | m-D1 | phenyl | Cz | phenyl | Cz |
| 1916 | Cz | phenyl | m-D1 | phenyl | Cz |
| 1917 | m-D1 | Cz | phenyl | phenyl | phenyl |
| 1918 | phenyl | m-D1 | phenyl | Cz | phenyl |
| 1919 | m-D1 | m-D1 | Cz | phenyl | Cz |
| 1920 | m-D1 | Cz | m-D1 | phenyl | Cz |
| 1921 | Cz | m-D1 | m-D1 | phenyl | Cz |
| 1922 | m-D1 | m-D1 | phenyl | Cz | Cz |
| 1923 | Cz | m-D1 | phenyl | m-D1 | Cz |
| 1924 | m-D1 | Cz | m-D1 | phenyl | phenyl |
| 1925 | phenyl | m-D1 | m-D1 | Cz | phenyl |
| 1926 | phenyl | m-D1 | Cz | m-D1 | phenyl |
| 1927 | m-D1 | phenyl | m-D1 | phenyl | Cz |
| 1928 | m-D1 | phenyl | Cz | phenyl | m-D1 |
| 1929 | phenyl | m-D1 | m-D1 | phenyl | phenyl |
| 1930 | m-D1 | m-D1 | m-D1 | Cz | phenyl |
| 1931 | m-D1 | m-D1 | Cz | m-D1 | phenyl |
| 1932 | m-D1 | m-D1 | m-D1 | phenyl | Cz |
| 1933 | m-D1 | m-D1 | phenyl | m-D1 | Cz |
| 1934 | m-D1 | m-D1 | phenyl | Cz | m-D1 |
| 1935 | m-D1 | m-D1 | phenyl | m-D1 | phenyl |
| 1936 | phenyl | m-D1 | m-D1 | m-D1 | phenyl |
| 1937 | m-D1 | m-D1 | m-D1 | m-D1 | phenyl |
| 1938 | m-D1 | m-D1 | phenyl | m-D1 | m-D1 |
| 1939 | m-D2 | Cz | Cz | Cz | phenyl |
| 1940 | Cz | Cz | Cz | m-D2 | phenyl |
| 1941 | m-D2 | Cz | Cz | phenyl | Cz |
| 1942 | Cz | Cz | m-D2 | phenyl | Cz |
| 1943 | Cz | m-D2 | phenyl | Cz | Cz |
| 1944 | Cz | m-D2 | Cz | phenyl | phenyl |
| 1945 | Cz | Cz | m-D2 | phenyl | phenyl |
| 1946 | phenyl | m-D2 | Cz | Cz | phenyl |
| 1947 | phenyl | Cz | m-D2 | Cz | phenyl |
| 1948 | m-D2 | phenyl | Cz | phenyl | Cz |
| 1949 | Cz | phenyl | m-D2 | phenyl | Cz |
| 1950 | m-D2 | Cz | phenyl | phenyl | phenyl |
| 1951 | phenyl | m-D2 | phenyl | Cz | phenyl |
| 1952 | m-D2 | m-D2 | Cz | phenyl | Cz |
| 1953 | m-D2 | Cz | m-D2 | phenyl | Cz |
| 1954 | Cz | m-D2 | m-D2 | phenyl | Cz |
| 1955 | m-D2 | m-D2 | phenyl | Cz | Cz |
| 1956 | Cz | m-D2 | phenyl | m-D2 | Cz |
| 1957 | m-D2 | Cz | m-D2 | phenyl | phenyl |
| 1958 | phenyl | m-D2 | m-D2 | Cz | phenyl |
| 1959 | phenyl | m-D2 | Cz | m-D2 | phenyl |
| 1960 | m-D2 | phenyl | m-D2 | phenyl | Cz |
| 1961 | m-D2 | phenyl | Cz | phenyl | m-D2 |
| 1962 | phenyl | m-D2 | m-D2 | phenyl | phenyl |
| 1963 | m-D2 | m-D2 | m-D2 | Cz | phenyl |
| 1964 | m-D2 | m-D2 | Cz | m-D2 | phenyl |
| 1965 | m-D2 | m-D2 | m-D2 | phenyl | Cz |
| 1966 | m-D2 | m-D2 | phenyl | m-D2 | Cz |
| 1967 | m-D2 | m-D2 | phenyl | Cz | m-D2 |
| 1968 | m-D2 | m-D2 | phenyl | m-D2 | phenyl |
| 1969 | phenyl | m-D2 | m-D2 | m-D2 | phenyl |
| 1970 | m-D2 | m-D2 | m-D2 | m-D2 | phenyl |
| 1971 | m-D2 | m-D2 | phenyl | m-D2 | m-D2 |
| 1972 | m-D3 | Cz | Cz | Cz | phenyl |
| 1973 | Cz | Cz | Cz | m-D3 | phenyl |
| 1974 | m-D3 | Cz | Cz | phenyl | Cz |
| 1975 | Cz | Cz | m-D3 | phenyl | Cz |
| 1976 | Cz | m-D3 | phenyl | Cz | Cz |
| 1977 | Cz | m-D3 | Cz | phenyl | phenyl |
| 1978 | Cz | Cz | m-D3 | phenyl | phenyl |
| 1979 | phenyl | m-D3 | Cz | Cz | phenyl |
| 1980 | phenyl | Cz | m-D3 | Cz | phenyl |
| 1981 | m-D3 | phenyl | Cz | phenyl | Cz |
| 1982 | Cz | phenyl | m-D3 | phenyl | Cz |
| 1983 | m-D3 | Cz | phenyl | phenyl | phenyl |
| 1984 | phenyl | m-D3 | phenyl | Cz | phenyl |
| 1985 | m-D3 | m-D3 | Cz | phenyl | Cz |
| 1986 | m-D3 | Cz | m-D3 | phenyl | Cz |
| 1987 | Cz | m-D3 | m-D3 | phenyl | Cz |
| 1988 | m-D3 | m-D3 | phenyl | Cz | Cz |
| 1989 | Cz | m-D3 | phenyl | m-D3 | Cz |
| 1990 | m-D3 | Cz | m-D3 | phenyl | phenyl |
| 1991 | phenyl | m-D3 | m-D3 | Cz | phenyl |
| 1992 | phenyl | m-D3 | Cz | m-D3 | phenyl |
| 1993 | m-D3 | phenyl | m-D3 | phenyl | Cz |
| 1994 | m-D3 | phenyl | Cz | phenyl | m-D3 |
| 1995 | phenyl | m-D3 | m-D3 | phenyl | phenyl |
| 1996 | m-D3 | m-D3 | m-D3 | Cz | phenyl |
| 1997 | m-D3 | m-D3 | Cz | m-D3 | phenyl |
| 1998 | m-D3 | m-D3 | m-D3 | phenyl | Cz |
| 1999 | m-D3 | m-D3 | phenyl | m-D3 | Cz |
| 2000 | m-D3 | m-D3 | phenyl | Cz | m-D3 |
| 2001 | m-D3 | m-D3 | phenyl | m-D3 | phenyl |
| 2002 | phenyl | m-D3 | m-D3 | m-D3 | phenyl |
| 2003 | m-D3 | m-D3 | m-D3 | m-D3 | phenyl |
| 2004 | m-D3 | m-D3 | phenyl | m-D3 | m-D3 |
| 2005 | m-D1 | Cz | Cz | Cz | 4-pyridyl |
| 2006 | Cz | Cz | Cz | m-D1 | 4-pyridyl |
| 2007 | m-D1 | Cz | Cz | 4-pyridyl | Cz |
| 2008 | Cz | Cz | m-D1 | 4-pyridyl | Cz |
| 2009 | Cz | m-D1 | 4-pyridyl | Cz | Cz |
| 2010 | Cz | m-D1 | Cz | 4-pyridyl | 4-pyridyl |
| 2011 | Cz | Cz | m-D1 | 4-pyridyl | 4-pyridyl |
| 2012 | 4-pyridyl | m-D1 | Cz | Cz | 4-pyridyl |
| 2013 | 4-pyridyl | Cz | m-D1 | Cz | 4-pyridyl |
| 2014 | m-D1 | 4-pyridyl | Cz | 4-pyridyl | Cz |
| 2015 | Cz | 4-pyridyl | m-D1 | 4-pyridyl | Cz |
| 2016 | m-D1 | Cz | 4-pyridyl | 4-pyridyl | 4-pyridyl |
| 2017 | 4-pyridyl | m-D1 | 4-pyridyl | Cz | 4-pyridyl |
| 2018 | m-D1 | m-D1 | Cz | 4-pyridyl | Cz |
| 2019 | m-D1 | Cz | m-D1 | 4-pyridyl | Cz |
| 2020 | Cz | m-D1 | m-D1 | 4-pyridyl | Cz |
| 2021 | m-D1 | m-D1 | 4-pyridyl | Cz | Cz |
| 2022 | Cz | m-D1 | 4-pyridyl | m-D1 | Cz |
| 2023 | m-D1 | Cz | m-D1 | 4-pyridyl | 4-pyridyl |
| 2024 | 4-pyridyl | m-D1 | m-D1 | Cz | 4-pyridyl |
| 2025 | 4-pyridyl | m-D1 | Cz | m-D1 | 4-pyridyl |
| 2026 | m-D1 | 4-pyridyl | m-D1 | 4-pyridyl | Cz |
| 2027 | m-D1 | 4-pyridyl | Cz | 4-pyridyl | m-D1 |
| 2028 | 4-pyridyl | m-D1 | m-D1 | 4-pyridyl | 4-pyridyl |
| 2029 | m-D1 | m-D1 | m-D1 | Cz | 4-pyridyl |
| 2030 | m-D1 | m-D1 | Cz | m-D1 | 4-pyridyl |
| 2031 | m-D1 | m-D1 | m-D1 | 4-pyridyl | Cz |
| 2032 | m-D1 | m-D1 | 4-pyridyl | m-D1 | Cz |
| 2033 | m-D1 | m-D1 | 4-pyridyl | Cz | m-D1 |
| 2034 | m-D1 | m-D1 | 4-pyridyl | m-D1 | 4-pyridyl |
| 2035 | 4-pyridyl | m-D1 | m-D1 | m-D1 | 4-pyridyl |
| 2036 | m-D1 | m-D1 | m-D1 | m-D1 | 4-pyridyl |
| 2037 | m-D1 | m-D1 | 4-pyridyl | m-D1 | m-D1 |
| 2038 | m-D2 | Cz | Cz | Cz | 4-pyridyl |
| 2039 | Cz | Cz | Cz | m-D2 | 4-pyridyl |
| 2040 | m-D2 | Cz | Cz | 4-pyridyl | Cz |
| 2041 | Cz | Cz | m-D2 | 4-pyridyl | Cz |
| 2042 | Cz | m-D2 | 4-pyridyl | Cz | Cz |
| 2043 | Cz | m-D2 | Cz | 4-pyridyl | 4-pyridyl |
| 2044 | Cz | Cz | m-D2 | 4-pyridyl | 4-pyridyl |
| 2045 | 4-pyridyl | m-D2 | Cz | Cz | 4-pyridyl |
| 2046 | 4-pyridyl | Cz | m-D2 | Cz | 4-pyridyl |
| 2047 | m-D2 | 4-pyridyl | Cz | 4-pyridyl | Cz |
| 2048 | Cz | 4-pyridyl | m-D2 | 4-pyridyl | Cz |
| 2049 | m-D2 | Cz | 4-pyridyl | 4-pyridyl | 4-pyridyl |
| 2050 | 4-pyridyl | m-D2 | 4-pyridyl | Cz | 4-pyridyl |
| 2051 | m-D2 | m-D2 | Cz | 4-pyridyl | Cz |
| 2052 | m-D2 | Cz | m-D2 | 4-pyridyl | Cz |
| 2053 | Cz | m-D2 | m-D2 | 4-pyridyl | Cz |
| 2054 | m-D2 | m-D2 | 4-pyridyl | Cz | Cz |
| 2055 | Cz | m-D2 | 4-pyridyl | m-D2 | Cz |
| 2056 | m-D2 | Cz | m-D2 | 4-pyridyl | 4-pyridyl |
| 2057 | 4-pyridyl | m-D2 | m-D2 | Cz | 4-pyridyl |
| 2058 | 4-pyridyl | m-D2 | Cz | m-D2 | 4-pyridyl |
| 2059 | m-D2 | 4-pyridyl | m-D2 | 4-pyridyl | Cz |
| 2060 | m-D2 | 4-pyridyl | Cz | 4-pyridyl | m-D2 |
| 2061 | 4-pyridyl | m-D2 | m-D2 | 4-pyridyl | 4-pyridyl |
| 2062 | m-D2 | m-D2 | m-D2 | Cz | 4-pyridyl |
| 2063 | m-D2 | m-D2 | Cz | m-D2 | 4-pyridyl |
| 2064 | m-D2 | m-D2 | m-D2 | 4-pyridyl | Cz |
| 2065 | m-D2 | m-D2 | 4-pyridyl | m-D2 | Cz |
| 2066 | m-D2 | m-D2 | 4-pyridyl | Cz | m-D2 |
| 2067 | m-D2 | m-D2 | 4-pyridyl | m-D2 | 4-pyridyl |
| 2068 | 4-pyridyl | m-D2 | m-D2 | m-D2 | 4-pyridyl |
| 2069 | m-D2 | m-D2 | m-D2 | m-D2 | 4-pyridyl |
| 2070 | m-D2 | m-D2 | 4-pyridyl | m-D2 | m-D2 |
| 2071 | m-D3 | Cz | Cz | Cz | 4-pyridyl |
| 2072 | Cz | Cz | Cz | m-D3 | 4-pyridyl |
| 2073 | m-D3 | Cz | Cz | 4-pyridyl | Cz |
| 2074 | Cz | Cz | m-D3 | 4-pyridyl | Cz |
| 2075 | Cz | m-D3 | 4-pyridyl | Cz | Cz |
| 2076 | Cz | m-D3 | Cz | 4-pyridyl | 4-pyridyl |
| 2077 | Cz | Cz | m-D3 | 4-pyridyl | 4-pyridyl |
| 2078 | 4-pyridyl | m-D3 | Cz | Cz | 4-pyridyl |
| 2079 | 4-pyridyl | Cz | m-D3 | Cz | 4-pyridyl |
| 2080 | m-D3 | 4-pyridyl | Cz | 4-pyridyl | Cz |
| 2081 | Cz | 4-pyridyl | m-D3 | 4-pyridyl | Cz |
| 2082 | m-D3 | Cz | 4-pyridyl | 4-pyridyl | 4-pyridyl |
| 2083 | 4-pyridyl | m-D3 | 4-pyridyl | Cz | 4-pyridyl |
| 2084 | m-D3 | m-D3 | Cz | 4-pyridyl | Cz |
| 2085 | m-D3 | Cz | m-D3 | 4-pyridyl | Cz |
| 2086 | Cz | m-D3 | m-D3 | 4-pyridyl | Cz |
| 2087 | m-D3 | m-D3 | 4-pyridyl | Cz | Cz |
| 2088 | Cz | m-D3 | 4-pyridyl | m-D3 | Cz |
| 2089 | m-D3 | Cz | m-D3 | 4-pyridyl | 4-pyridyl |
| 2090 | 4-pyridyl | m-D3 | m-D3 | Cz | 4-pyridyl |
| 2091 | 4-pyridyl | m-D3 | Cz | m-D3 | 4-pyridyl |
| 2092 | m-D3 | 4-pyridyl | m-D3 | 4-pyridyl | Cz |
| 2093 | m-D3 | 4-pyridyl | Cz | 4-pyridyl | m-D3 |
| 2094 | 4-pyridyl | m-D3 | m-D3 | 4-pyridyl | 4-pyridyl |
| 2095 | m-D3 | m-D3 | m-D3 | Cz | 4-pyridyl |
| 2096 | m-D3 | m-D3 | Cz | m-D3 | 4-pyridyl |
| 2097 | m-D3 | m-D3 | m-D3 | 4-pyridyl | Cz |
| 2098 | m-D3 | m-D3 | 4-pyridyl | m-D3 | Cz |
| 2099 | m-D3 | m-D3 | 4-pyridyl | Cz | m-D3 |
| 2100 | m-D3 | m-D3 | 4-pyridyl | m-D3 | 4-pyridyl |
| 2101 | 4-pyridyl | m-D3 | m-D3 | m-D3 | 4-pyridyl |
| 2102 | m-D3 | m-D3 | m-D3 | m-D3 | 4-pyridyl |
| 2103 | m-D3 | m-D3 | 4-pyridyl | m-D3 | m-D3 |
| 2104 | m-D1 | Cz | Cz | Cz | methoxy |
| 2105 | Cz | Cz | Cz | m-D1 | methoxy |
| 2106 | m-D1 | Cz | Cz | methoxy | Cz |
| 2107 | Cz | Cz | m-D1 | methoxy | Cz |
| 2108 | Cz | m-D1 | methoxy | Cz | Cz |
| 2109 | Cz | m-D1 | Cz | methoxy | methoxy |
| 2110 | Cz | Cz | m-D1 | methoxy | methoxy |
| 2111 | methoxy | m-D1 | Cz | Cz | methoxy |
| 2112 | methoxy | Cz | m-D1 | Cz | methoxy |
| 2113 | m-D1 | methoxy | Cz | methoxy | Cz |
| 2114 | Cz | methoxy | m-D1 | methoxy | Cz |
| 2115 | m-D1 | Cz | methoxy | methoxy | methoxy |
| 2116 | methoxy | m-D1 | methoxy | Cz | methoxy |
| 2117 | m-D1 | m-D1 | Cz | methoxy | Cz |
| 2118 | m-D1 | Cz | m-D1 | methoxy | Cz |
| 2119 | Cz | m-D1 | m-D1 | methoxy | Cz |
| 2120 | m-D1 | m-D1 | methoxy | Cz | Cz |
| 2121 | Cz | m-D1 | methoxy | m-D1 | Cz |
| 2122 | m-D1 | Cz | m-D1 | methoxy | methoxy |
| 2123 | methoxy | m-D1 | m-D1 | Cz | methoxy |
| 2124 | methoxy | m-D1 | Cz | m-D1 | methoxy |
| 2125 | m-D1 | methoxy | m-D1 | methoxy | Cz |
| 2126 | m-D1 | methoxy | Cz | methoxy | m-D1 |
| 2127 | methoxy | m-D1 | m-D1 | methoxy | methoxy |
| 2128 | m-D1 | m-D1 | m-D1 | Cz | methoxy |
| 2129 | m-D1 | m-D1 | Cz | m-D1 | methoxy |
| 2130 | m-D1 | m-D1 | m-D1 | methoxy | Cz |
| 2131 | m-D1 | m-D1 | methoxy | m-D1 | Cz |
| 2132 | m-D1 | m-D1 | methoxy | Cz | m-D1 |
| 2133 | m-D1 | m-D1 | methoxy | m-D1 | methoxy |
| 2134 | methoxy | m-D1 | m-D1 | m-D1 | methoxy |
| 2135 | m-D1 | m-D1 | m-D1 | m-D1 | methoxy |
| 2136 | m-D1 | m-D1 | methoxy | m-D1 | m-D1 |
| 2137 | m-D2 | Cz | Cz | Cz | methoxy |
| 2138 | Cz | Cz | Cz | m-D2 | methoxy |
| 2139 | m-D2 | Cz | Cz | methoxy | Cz |
| 2140 | Cz | Cz | m-D2 | methoxy | Cz |
| 2141 | Cz | m-D2 | methoxy | Cz | Cz |
| 2142 | Cz | m-D2 | Cz | methoxy | methoxy |
| 2143 | Cz | Cz | m-D2 | methoxy | methoxy |
| 2144 | methoxy | m-D2 | Cz | Cz | methoxy |
| 2145 | methoxy | Cz | m-D2 | Cz | methoxy |
| 2146 | m-D2 | methoxy | Cz | methoxy | Cz |
| 2147 | Cz | methoxy | m-D2 | methoxy | Cz |
| 2148 | m-D2 | Cz | methoxy | methoxy | methoxy |
| 2149 | methoxy | m-D2 | methoxy | Cz | methoxy |
| 2150 | m-D2 | m-D2 | Cz | methoxy | Cz |
| 2151 | m-D2 | Cz | m-D2 | methoxy | Cz |
| 2152 | Cz | m-D2 | m-D2 | methoxy | Cz |
| 2153 | m-D2 | m-D2 | methoxy | Cz | Cz |
| 2154 | Cz | m-D2 | methoxy | m-D2 | Cz |
| 2155 | m-D2 | Cz | m-D2 | methoxy | methoxy |
| 2156 | methoxy | m-D2 | m-D2 | Cz | methoxy |
| 2157 | methoxy | m-D2 | Cz | m-D2 | methoxy |
| 2158 | m-D2 | methoxy | m-D2 | methoxy | Cz |
| 2159 | m-D2 | methoxy | Cz | methoxy | m-D2 |
| 2160 | methoxy | m-D2 | m-D2 | methoxy | methoxy |
| 2161 | m-D2 | m-D2 | m-D2 | Cz | methoxy |
| 2162 | m-D2 | m-D2 | Cz | m-D2 | methoxy |
| 2163 | m-D2 | m-D2 | m-D2 | methoxy | Cz |
| 2164 | m-D2 | m-D2 | methoxy | m-D2 | Cz |
| 2165 | m-D2 | m-D2 | methoxy | Cz | m-D2 |
| 2166 | m-D2 | m-D2 | methoxy | m-D2 | methoxy |
| 2167 | methoxy | m-D2 | m-D2 | m-D2 | methoxy |
| 2168 | m-D2 | m-D2 | m-D2 | m-D2 | methoxy |
| 2169 | m-D2 | m-D2 | methoxy | m-D2 | m-D2 |
| 2170 | m-D3 | Cz | Cz | Cz | methoxy |
| 2171 | Cz | Cz | Cz | m-D3 | methoxy |
| 2172 | m-D3 | Cz | Cz | methoxy | Cz |
| 2173 | Cz | Cz | m-D3 | methoxy | Cz |
| 2174 | Cz | m-D3 | methoxy | Cz | Cz |
| 2175 | Cz | m-D3 | Cz | methoxy | methoxy |
| 2176 | Cz | Cz | m-D3 | methoxy | methoxy |
| 2177 | methoxy | m-D3 | Cz | Cz | methoxy |
| 2178 | methoxy | Cz | m-D3 | Cz | methoxy |
| 2179 | m-D3 | methoxy | Cz | methoxy | Cz |
| 2180 | Cz | methoxy | m-D3 | methoxy | Cz |
| 2181 | m-D3 | Cz | methoxy | methoxy | methoxy |
| 2182 | methoxy | m-D3 | methoxy | Cz | methoxy |
| 2183 | m-D3 | m-D3 | Cz | methoxy | Cz |
| 2184 | m-D3 | Cz | m-D3 | methoxy | Cz |
| 2185 | Cz | m-D3 | m-D3 | methoxy | Cz |
| 2186 | m-D3 | m-D3 | methoxy | Cz | Cz |
| 2187 | Cz | m-D3 | methoxy | m-D3 | Cz |
| 2188 | m-D3 | Cz | m-D3 | methoxy | methoxy |
| 2189 | methoxy | m-D3 | m-D3 | Cz | methoxy |
| 2190 | methoxy | m-D3 | Cz | m-D3 | methoxy |
| 2191 | m-D3 | methoxy | m-D3 | methoxy | Cz |
| 2192 | m-D3 | methoxy | Cz | methoxy | m-D3 |
| 2193 | methoxy | m-D3 | m-D3 | methoxy | methoxy |
| 2194 | m-D3 | m-D3 | m-D3 | Cz | methoxy |
| 2195 | m-D3 | m-D3 | Cz | m-D3 | methoxy |
| 2196 | m-D3 | m-D3 | m-D3 | methoxy | Cz |
| 2197 | m-D3 | m-D3 | methoxy | m-D3 | Cz |
| 2198 | m-D3 | m-D3 | methoxy | Cz | m-D3 |
| 2199 | m-D3 | m-D3 | methoxy | m-D3 | methoxy |
| 2200 | methoxy | m-D3 | m-D3 | m-D3 | methoxy |
| 2201 | m-D3 | m-D3 | m-D3 | m-D3 | methoxy |
| 2202 | m-D3 | m-D3 | methoxy | m-D3 | m-D3 |
| 2203 | Cz | m-D1 | Cz | H | m-D2 |
| 2204 | Cz | m-D2 | Cz | H | m-D1 |
| 2205 | m-D1 | Cz | H | m-D2 | Cz |
| 2206 | m-D2 | Cz | H | m-D3 | Cz |
| 2207 | m-D1 | m-D2 | H | Cz | H |
| 2208 | m-D2 | m-D3 | H | Cz | H |
| 2209 | m-D1 | Cz | H | m-D2 | H |
| 2210 | m-D2 | Cz | H | m-D1 | H |
| 2211 | Cz | m-D1 | H | m-D2 | H |
| 2212 | Cz | m-D2 | H | m-D1 | H |
| 2213 | m-D1 | H | m-D2 | Cz | H |
| 2214 | m-D2 | H | m-D3 | H | Cz |
| 2215 | m-D1 | H | m-D2 | H | Cz |
| 2216 | m-D1 | H | m-D3 | H | Cz |
| 2217 | m-D1 | H | Cz | H | m-D2 |
| 2218 | m-D1 | H | Cz | H | m-D3 |
| 2219 | m-D2 | H | Cz | H | m-D3 |
| 2220 | m-D1 | H | H | m-D2 | H |
| 2221 | m-D2 | H | H | m-D3 | H |
| 2222 | H | m-D1 | H | m-D3 | H |
| 2223 | m-D1 | H | H | H | m-D2 |
| 2224 | m-D1 | H | H | H | m-D3 |
| 2225 | m-D2 | H | H | H | m-D3 |
| 2226 | m-D1 | H | m-D2 | H | m-D1 |
| 2227 | m-D2 | H | m-D3 | H | m-D2 |
| 2228 | m-D2 | H | m-D3 | H | m-D2 |
| 2229 | m-D3 | H | m-D2 | H | m-D3 |
| 2230 | m-D1 | m-D2 | m-D3 | H | H |
| 2231 | m-D1 | m-D2 | m-D1 | H | H |
| 2232 | m-D1 | m-D3 | H | m-D3 | H |
| 2233 | m-D1 | m-D2 | H | m-D3 | H |
| 2234 | m-D1 | m-D2 | m-D3 | H | m-D4 |
| 2235 | m-D1 | m-D1 | m-D2 | H | m-D4 |
| 2236 | m-D2 | m-D2 | H | m-D3 | m-D2 |
| 2237 | Cz | Cz | m-D1 | H | phenyl |
| 2238 | H | m-D1 | Cz | Cz | methyl |
| 2239 | m-D1 | H | Cz | metoxy | Cz |
| 2240 | Cz | methyl | m-D1 | phenyl | Cz |
| 2241 | m-D1 | H | Cz | H | methyl |
| 2242 | Cz | methyl | m-D1 | H | H |
| 2243 | methyl | m-D1 | H | Cz | H |
| 2244 | m-D1 | H | m-D1 | Cz | methyl |
| 2245 | m-D1 | methyl | m-D1 | H | Cz |
| 2246 | m-D1 | H | Cz | methoxy | m-D1 |
| 2247 | m-D1 | H | m-D1 | H | methyl |
| 2248 | H | m-D1 | methyl | m-D1 | H |
| 2249 | H | m-D1 | phenyl | m-D1 | H |
| 2250 | m-D1 | H | methyl | H | m-D1 |
| 2251 | m-D1 | H | phenyl | H | m-D1 |
| 2252 | m-D1 | H | methoxy | H | m-D1 |
| 2253 | m-D1 | methyl | phenyl | methoxy | m-D1 |
| 2254 | phenyl | H | m-D1 | H | phneyl |
| 2255 | 4-pyridyl | H | m-D1 | H | 4-pyridyl |
| 2256 | Cz | Cz | m-D2 | H | phenyl |
| 2257 | H | m-D2 | Cz | Cz | methyl |
| 2258 | m-D2 | H | Cz | metoxy | Cz |
| 2259 | Cz | methyl | m-D2 | phenyl | Cz |
| 2260 | m-D2 | H | Cz | H | methyl |
| 2261 | Cz | methyl | m-D2 | H | H |
| 2262 | methyl | m-D2 | H | Cz | H |
| 2263 | m-D2 | H | m-D2 | Cz | methyl |
| 2264 | m-D2 | methyl | m-D2 | H | Cz |
| 2265 | m-D2 | H | Cz | methoxy | m-D2 |
| 2266 | m-D2 | H | m-D2 | H | methyl |
| 2267 | H | m-D2 | methyl | m-D2 | H |
| 2268 | H | m-D2 | phenyl | m-D2 | H |
| 2269 | m-D2 | H | methyl | H | m-D2 |
| 2270 | m-D2 | H | phenyl | H | m-D2 |
| 2271 | m-D2 | H | methoxy | H | m-D2 |
| 2272 | m-D2 | methyl | phenyl | methoxy | m-D2 |
| 2273 | phenyl | H | m-D2 | H | phneyl |
| 2274 | 4-pyridyl | H | m-D2 | H | 4-pyridyl |
| 2275 | Cz | Cz | m-D3 | H | phenyl |
| 2276 | H | m-D3 | Cz | Cz | methyl |
| 2277 | m-D3 | H | Cz | metoxy | Cz |
| 2278 | Cz | methyl | m-D3 | phenyl | Cz |
| 2279 | m-D3 | H | Cz | H | methyl |
| 2280 | Cz | methyl | m-D3 | H | H |
| 2281 | methyl | m-D3 | H | Cz | H |
| 2282 | m-D3 | H | m-D3 | Cz | methyl |
| 2283 | m-D3 | methyl | m-D3 | H | Cz |
| 2284 | m-D3 | H | Cz | methoxy | m-D3 |
| 2285 | m-D3 | H | m-D3 | H | methyl |
| 2286 | H | m-D3 | methyl | m-D3 | H |
| 2287 | H | m-D3 | phenyl | m-D3 | H |
| 2288 | m-D3 | H | methyl | H | m-D3 |
| 2289 | m-D3 | H | phenyl | H | m-D3 |
| 2290 | m-D3 | H | methoxy | H | m-D3 |
| 2291 | m-D3 | methyl | phenyl | methoxy | m-D3 |
| 2292 | phenyl | H | m-D3 | H | phneyl |
| 2293 | 4-pyridyl | H | m-D3 | H | 4-pyridyl |
| 2294 | Cz9 | Cz9 | m-D2 | Cz9 | Cz9 |
| 2295 | m-D2 | Cz9 | Cz9 | Cz9 | H |
| 2296 | Cz9 | Cz9 | Cz9 | m-D2 | H |
| 2297 | m-D2 | Cz9 | Cz9 | H | Cz9 |
| 2298 | Cz9 | Cz9 | m-D2 | H | Cz9 |
| 2299 | Cz9 | m-D2 | H | Cz9 | Cz9 |
| 2300 | Cz9 | m-D2 | Cz9 | H | H |
| 2301 | Cz9 | Cz9 | m-D2 | H | H |
| 2302 | H | m-D2 | Cz9 | Cz9 | H |
| 2303 | H | Cz9 | m-D2 | Cz9 | H |
| 2304 | m-D2 | H | Cz9 | H | Cz9 |
| 2305 | Cz9 | H | m-D2 | H | Cz9 |
| 2306 | m-D2 | Cz9 | H | H | H |
| 2307 | H | m-D2 | H | Cz9 | H |
| 2308 | m-D2 | m-D2 | Cz9 | H | Cz9 |
| 2309 | m-D2 | Cz9 | m-D2 | H | Cz9 |
| 2310 | Cz9 | m-D2 | m-D2 | H | Cz9 |
| 2311 | m-D2 | m-D2 | H | Cz9 | Cz9 |
| 2312 | Cz9 | m-D2 | H | m-D2 | Cz9 |
| 2313 | m-D2 | Cz9 | m-D2 | H | H |
| 2314 | H | m-D2 | m-D2 | Cz9 | H |
| 2315 | H | m-D2 | Cz9 | m-D2 | H |
| 2316 | m-D2 | H | m-D2 | H | Cz9 |
| 2317 | m-D2 | H | Cz9 | H | m-D2 |
| 2318 | m-D2 | Cz9 | m-D2 | Cz9 | m-D2 |
| 2319 | m-D2 | m-D2 | m-D2 | Cz9 | H |
| 2320 | m-D2 | m-D2 | Cz9 | m-D2 | H |
| 2321 | m-D2 | m-D2 | m-D2 | H | Cz9 |
| 2322 | m-D2 | m-D2 | H | m-D2 | Cz9 |
| 2323 | m-D2 | m-D2 | H | Cz9 | m-D2 |
| 2324 | m-D2 | m-D2 | m-D2 | m-D2 | Cz9 |
| 2325 | Cz9 | Cz9 | m-D3 | Cz9 | Cz9 |
| 2326 | m-D3 | Cz9 | Cz9 | Cz9 | H |
| 2327 | Cz9 | Cz9 | Cz9 | m-D3 | H |
| 2328 | m-D3 | Cz9 | Cz9 | H | Cz9 |
| 2329 | Cz9 | Cz9 | m-D3 | H | Cz9 |
| 2330 | Cz9 | m-D3 | H | Cz9 | Cz9 |
| 2331 | Cz9 | m-D3 | Cz9 | H | H |
| 2332 | Cz9 | Cz9 | m-D3 | H | H |
| 2333 | H | m-D3 | Cz9 | Cz9 | H |
| 2334 | H | Cz9 | m-D3 | Cz9 | H |
| 2335 | m-D3 | H | Cz9 | H | Cz9 |
| 2336 | Cz9 | H | m-D3 | H | Cz9 |
| 2337 | m-D3 | Cz9 | H | H | H |
| 2338 | H | m-D3 | H | Cz9 | H |
| 2339 | m-D3 | m-D3 | Cz9 | H | Cz9 |
| 2340 | m-D3 | Cz9 | m-D3 | H | Cz9 |
| 2341 | Cz9 | m-D3 | m-D3 | H | Cz9 |
| 2342 | m-D3 | m-D3 | H | Cz9 | Cz9 |
| 2343 | Cz9 | m-D3 | H | m-D3 | Cz9 |
| 2344 | m-D3 | Cz9 | m-D3 | H | H |
| 2345 | H | m-D3 | m-D3 | Cz9 | H |
| 2346 | H | m-D3 | Cz9 | m-D3 | H |
| 2347 | m-D3 | H | m-D3 | H | Cz9 |
| 2348 | m-D3 | H | Cz9 | H | m-D3 |
| 2349 | m-D3 | Cz9 | m-D3 | Cz9 | m-D3 |
| 2350 | m-D3 | m-D3 | m-D3 | Cz9 | H |
| 2351 | m-D3 | m-D3 | Cz9 | m-D3 | H |
| 2352 | m-D3 | m-D3 | m-D3 | H | Cz9 |
| 2353 | m-D3 | m-D3 | H | m-D3 | Cz9 |
| 2354 | m-D3 | m-D3 | H | Cz9 | m-D3 |
| 2355 | m-D3 | m-D3 | m-D3 | m-D3 | Cz9 |
| 2356 | Cz10 | Cz10 | m-D2 | Cz10 | Cz10 |
| 2357 | m-D2 | Cz10 | Cz10 | Cz10 | H |
| 2358 | Cz10 | Cz10 | Cz10 | m-D2 | H |
| 2359 | m-D2 | Cz10 | Cz10 | H | Cz10 |
| 2360 | Cz10 | Cz10 | m-D2 | H | Cz10 |
| 2361 | Cz10 | m-D2 | H | Cz10 | Cz10 |
| 2362 | Cz10 | m-D2 | Cz10 | H | H |
| 2363 | Cz10 | Cz10 | m-D2 | H | H |
| 2364 | H | m-D2 | Cz10 | Cz10 | H |
| 2365 | H | Cz10 | m-D2 | Cz10 | H |
| 2366 | m-D2 | H | Cz10 | H | Cz10 |
| 2367 | Cz10 | H | m-D2 | H | Cz10 |
| 2368 | m-D2 | Cz10 | H | H | H |
| 2369 | H | m-D2 | H | Cz10 | H |
| 2370 | m-D2 | m-D2 | Cz10 | H | Cz10 |
| 2371 | m-D2 | Cz10 | m-D2 | H | Cz10 |
| 2372 | Cz10 | m-D2 | m-D2 | H | Cz10 |
| 2373 | m-D2 | m-D2 | H | Cz10 | Cz10 |
| 2374 | Cz10 | m-D2 | H | m-D2 | Cz10 |
| 2375 | m-D2 | Cz10 | m-D2 | H | H |
| 2376 | H | m-D2 | m-D2 | Cz10 | H |
| 2377 | H | m-D2 | Cz10 | m-D2 | H |
| 2378 | m-D2 | H | m-D2 | H | Cz10 |
| 2379 | m-D2 | H | Cz10 | H | m-D2 |
| 2380 | m-D2 | Cz10 | m-D2 | Cz10 | m-D2 |
| 2381 | m-D2 | m-D2 | m-D2 | Cz10 | H |
| 2382 | m-D2 | m-D2 | Cz10 | m-D2 | H |
| 2383 | m-D2 | m-D2 | m-D2 | H | Cz10 |
| 2384 | m-D2 | m-D2 | H | m-D2 | Cz10 |
| 2385 | m-D2 | m-D2 | H | Cz10 | m-D2 |
| 2386 | m-D2 | m-D2 | m-D2 | m-D2 | Cz10 |
| 2387 | Cz10 | Cz10 | m-D3 | Cz10 | Cz10 |
| 2388 | m-D3 | Cz10 | Cz10 | Cz10 | H |
| 2389 | Cz10 | Cz10 | Cz10 | m-D3 | H |
| 2390 | m-D3 | Cz10 | Cz10 | H | Cz10 |
| 2391 | Cz10 | Cz10 | m-D3 | H | Cz10 |
| 2392 | Cz10 | m-D3 | H | Cz10 | Cz10 |
| 2393 | Cz10 | m-D3 | Cz10 | H | H |
| 2394 | Cz10 | Cz10 | m-D3 | H | H |
| 2395 | H | m-D3 | Cz10 | Cz10 | H |
| 2396 | H | Cz10 | m-D3 | Cz10 | H |
| 2397 | m-D3 | H | Cz10 | H | Cz10 |
| 2398 | Cz10 | H | m-D3 | H | Cz10 |
| 2399 | m-D3 | Cz10 | H | H | H |
| 2400 | H | m-D3 | H | Cz10 | H |
| 2401 | m-D3 | m-D3 | Cz10 | H | Cz10 |
| 2402 | m-D3 | Cz10 | m-D3 | H | Cz10 |
| 2403 | Cz10 | m-D3 | m-D3 | H | Cz10 |
| 2404 | m-D3 | m-D3 | H | Cz10 | Cz10 |
| 2405 | Cz10 | m-D3 | H | m-D3 | Cz10 |
| 2406 | m-D3 | Cz10 | m-D3 | H | H |
| 2407 | H | m-D3 | m-D3 | Cz10 | H |
| 2408 | H | m-D3 | Cz10 | m-D3 | H |
| 2409 | m-D3 | H | m-D3 | H | Cz10 |
| 2410 | m-D3 | H | Cz10 | H | m-D3 |
| 2411 | m-D3 | Cz10 | m-D3 | Cz10 | m-D3 |
| 2412 | m-D3 | m-D3 | m-D3 | Cz10 | H |
| 2413 | m-D3 | m-D3 | Cz10 | m-D3 | H |
| 2414 | m-D3 | m-D3 | m-D3 | H | Cz10 |
| 2415 | m-D3 | m-D3 | H | m-D3 | Cz10 |
| 2416 | m-D3 | m-D3 | H | Cz10 | m-D3 |
| 2417 | m-D3 | m-D3 | m-D3 | m-D3 | Cz10 |
| 2418 | Cz11 | Cz11 | m-D2 | Cz11 | Cz11 |
| 2419 | m-D2 | Cz11 | Cz11 | Cz11 | H |
| 2420 | Cz11 | Cz11 | Cz11 | m-D2 | H |
| 2421 | m-D2 | Cz11 | Cz11 | H | Cz11 |
| 2422 | Cz11 | Cz11 | m-D2 | H | Cz11 |
| 2423 | Cz11 | m-D2 | H | Cz11 | Cz11 |
| 2424 | Cz11 | m-D2 | Cz11 | H | H |
| 2425 | Cz11 | Cz11 | m-D2 | H | H |
| 2426 | H | m-D2 | Cz11 | Cz11 | H |
| 2427 | H | Cz11 | m-D2 | Cz11 | H |
| 2428 | m-D2 | H | Cz11 | H | Cz11 |
| 2429 | Cz11 | H | m-D2 | H | Cz11 |
| 2430 | m-D2 | Cz11 | H | H | H |
| 2431 | H | m-D2 | H | Cz11 | H |
| 2432 | m-D2 | m-D2 | Cz11 | H | Cz11 |
| 2433 | m-D2 | Cz11 | m-D2 | H | Cz11 |
| 2434 | Cz11 | m-D2 | m-D2 | H | Cz11 |
| 2435 | m-D2 | m-D2 | H | Cz11 | Cz11 |
| 2436 | Cz11 | m-D2 | H | m-D2 | Cz11 |
| 2437 | m-D2 | Cz11 | m-D2 | H | H |
| 2438 | H | m-D2 | m-D2 | Cz11 | H |
| 2439 | H | m-D2 | Cz11 | m-D2 | H |
| 2440 | m-D2 | H | m-D2 | H | Cz11 |
| 2441 | m-D2 | H | Cz11 | H | m-D2 |
| 2442 | m-D2 | Cz11 | m-D2 | Cz11 | m-D2 |
| 2443 | m-D2 | m-D2 | m-D2 | Cz11 | H |
| 2444 | m-D2 | m-D2 | Cz11 | m-D2 | H |
| 2445 | m-D2 | m-D2 | m-D2 | H | Cz11 |
| 2446 | m-D2 | m-D2 | H | m-D2 | Cz11 |
| 2445 | m-D2 | m-D2 | H | Cz11 | m-D2 |
| 2448 | m-D2 | m-D2 | m-D2 | m-D2 | Cz11 |
| 2449 | Cz11 | Cz11 | m-D3 | Cz11 | Cz11 |
| 2450 | m-D3 | Cz11 | Cz11 | Cz11 | H |
| 2451 | Cz11 | Cz11 | Cz11 | m-D3 | H |
| 2452 | m-D3 | Cz11 | Cz11 | H | Cz11 |
| 2453 | Cz11 | Cz11 | m-D3 | H | Cz11 |
| 2454 | Cz11 | m-D3 | H | Cz11 | Cz11 |
| 2455 | Cz11 | m-D3 | Cz11 | H | H |
| 2456 | Cz11 | Cz11 | m-D3 | H | H |
| 2457 | H | m-D3 | Cz11 | Cz11 | H |
| 2458 | H | Cz11 | m-D3 | Cz11 | H |
| 2459 | m-D3 | H | Cz11 | H | Cz11 |
| 2460 | Cz11 | H | m-D3 | H | Cz11 |
| 2461 | m-D3 | Cz11 | H | H | H |
| 2462 | H | m-D3 | H | Cz11 | H |
| 2463 | m-D3 | m-D3 | Cz11 | H | Cz11 |
| 2464 | m-D3 | Cz11 | m-D3 | H | Cz11 |
| 2465 | Cz11 | m-D3 | m-D3 | H | Cz11 |
| 2466 | m-D3 | m-D3 | H | Cz11 | Cz11 |
| 2467 | Cz11 | m-D3 | H | m-D3 | Cz11 |
| 2468 | m-D3 | Cz11 | m-D3 | H | H |
| 2469 | H | m-D3 | m-D3 | Cz11 | H |
| 2470 | H | m-D3 | Cz11 | m-D3 | H |
| 2471 | m-D3 | H | m-D3 | H | Cz11 |
| 2472 | m-D3 | H | Cz11 | H | m-D3 |
| 2473 | m-D3 | Cz11 | m-D3 | Cz11 | m-D3 |
| 2474 | m-D3 | m-D3 | m-D3 | Cz11 | H |
| 2475 | m-D3 | m-D3 | Cz11 | m-D3 | H |
| 2476 | m-D3 | m-D3 | m-D3 | H | Cz11 |
| 2477 | m-D3 | m-D3 | H | m-D3 | Cz11 |
| 2478 | m-D3 | m-D3 | H | Cz11 | m-D3 |
| 2479 | m-D3 | m-D3 | m-D3 | m-D3 | Cz11 |
| 2480 | m-D1 | Cz12 | Cz12 | Cz12 | Cz12 |
| 2481 | Cz12 | m-D1 | Cz12 | Cz12 | Cz12 |
| 2482 | Cz12 | Cz12 | m-D1 | Cz12 | Cz12 |
| 2483 | m-D1 | Cz12 | Cz12 | Cz12 | H |
| 2484 | Cz12 | m-D1 | Cz12 | Cz12 | H |
| 2485 | Cz12 | Cz12 | m-D1 | Cz12 | H |
| 2486 | Cz12 | Cz12 | Cz12 | m-D1 | H |
| 2487 | m-D1 | Cz12 | Cz12 | H | Cz12 |
| 2488 | Cz12 | m-D1 | Cz12 | H | Cz12 |
| 2489 | Cz12 | Cz12 | m-D1 | H | Cz12 |
| 2490 | Cz12 | Cz12 | Cz12 | H | m-D1 |
| 2491 | m-D1 | Cz12 | H | Cz12 | Cz12 |
| 2492 | Cz12 | m-D1 | H | Cz12 | Cz12 |
| 2493 | m-D1 | Cz12 | Cz12 | H | H |
| 2494 | Cz12 | m-D1 | Cz12 | H | H |
| 2495 | Cz12 | Cz12 | m-D1 | H | H |
| 2496 | m-D1 | Cz12 | H | Cz12 | H |
| 2497 | Cz12 | m-D1 | H | Cz12 | H |
| 2498 | Cz12 | Cz12 | H | m-D1 | H |
| 2499 | m-D1 | H | Cz12 | Cz12 | H |
| 2500 | Cz12 | H | m-D1 | Cz12 | H |
| 2501 | Cz12 | H | Cz12 | m-D1 | H |
| 2502 | H | m-D1 | Cz12 | Cz12 | H |
| 2503 | H | Cz12 | m-D1 | Cz12 | H |
| 2504 | m-D1 | Cz12 | H | H | Cz12 |
| 2505 | Cz12 | m-D1 | H | H | Cz12 |
| 2506 | Cz12 | Cz12 | H | H | m-D1 |
| 2507 | m-D1 | H | Cz12 | H | Cz12 |
| 2508 | Cz12 | H | m-D1 | H | Cz12 |
| 2509 | m-D1 | Cz12 | H | H | H |
| 2510 | Cz12 | m-D1 | H | H | H |
| 2511 | m-D1 | H | Cz12 | H | H |
| 2512 | Cz12 | H | m-D1 | H | H |
| 2513 | H | m-D1 | Cz12 | H | H |
| 2514 | H | Cz12 | m-D1 | H | H |
| 2515 | m-D1 | H | H | Cz12 | H |
| 2516 | Cz12 | H | H | m-D1 | H |
| 2517 | H | m-D1 | H | Cz12 | H |
| 2518 | m-D1 | H | H | H | Cz12 |
| 2519 | m-D1 | m-D1 | Cz12 | Cz12 | H |
| 2520 | m-D1 | Cz12 | m-D1 | Cz12 | H |
| 2521 | m-D1 | Cz12 | Cz12 | m-D1 | H |
| 2522 | Cz12 | m-D1 | m-D1 | Cz12 | H |
| 2523 | Cz12 | m-D1 | Cz12 | m-D1 | H |
| 2524 | Cz12 | Cz12 | m-D1 | m-D1 | H |
| 2525 | m-D1 | m-D1 | Cz12 | H | Cz12 |
| 2526 | m-D1 | Cz12 | m-D1 | H | Cz12 |
| 2527 | m-D1 | Cz12 | Cz12 | H | m-D1 |
| 2528 | Cz12 | m-D1 | m-D1 | H | Cz12 |
| 2529 | Cz12 | m-D1 | Cz12 | H | m-D1 |
| 2530 | Cz12 | Cz12 | m-D1 | H | m-D1 |
| 2531 | m-D1 | m-D1 | H | Cz12 | Cz12 |
| 2532 | m-D1 | Cz12 | H | m-D1 | Cz12 |
| 2533 | m-D1 | Cz12 | H | Cz12 | m-D1 |
| 2534 | Cz12 | m-D1 | H | m-D1 | Cz12 |
| 2535 | m-D1 | m-D1 | Cz12 | H | H |
| 2536 | m-D1 | Cz12 | m-D1 | H | H |
| 2537 | Cz12 | m-D1 | m-D1 | H | H |
| 2538 | m-D1 | m-D1 | H | Cz12 | H |
| 2539 | m-D1 | Cz12 | H | m-D1 | H |
| 2540 | Cz12 | m-D1 | H | m-D1 | H |
| 2541 | m-D1 | H | m-D1 | Cz12 | H |
| 2542 | m-D1 | H | Cz12 | m-D1 | H |
| 2543 | Cz12 | H | m-D1 | m-D1 | H |
| 2544 | H | m-D1 | m-D1 | Cz12 | H |
| 2545 | H | m-D1 | Cz12 | m-D1 | H |
| 2546 | m-D1 | m-D1 | H | H | Cz12 |
| 2547 | m-D1 | Cz12 | H | H | m-D1 |
| 2548 | Cz12 | m-D1 | H | H | m-D1 |
| 2549 | m-D1 | H | m-D1 | H | Cz12 |
| 2550 | m-D1 | H | Cz12 | H | m-D1 |
| 2551 | m-D1 | m-D1 | m-D1 | Cz12 | Cz12 |
| 2552 | m-D1 | m-D1 | Cz12 | m-D1 | Cz12 |
| 2553 | m-D1 | m-D1 | Cz12 | Cz12 | m-D1 |
| 2554 | m-D1 | Cz12 | m-D1 | m-D1 | Cz12 |
| 2555 | m-D1 | Cz12 | m-D1 | Cz12 | m-D1 |
| 2556 | m-D1 | m-D1 | m-D1 | Cz12 | H |
| 2557 | m-D1 | m-D1 | Cz12 | m-D1 | H |
| 2558 | m-D1 | Cz12 | m-D1 | Cz12 | H |
| 2559 | Cz12 | m-D1 | m-D1 | m-D1 | H |
| 2560 | m-D1 | m-D1 | m-D1 | H | Cz12 |
| 2561 | m-D1 | m-D1 | Cz12 | H | m-D1 |
| 2562 | m-D1 | Cz12 | m-D1 | H | m-D1 |
| 2563 | Cz12 | m-D1 | m-D1 | H | m-D1 |
| 2564 | m-D1 | m-D1 | H | m-D1 | Cz12 |
| 2565 | m-D1 | m-D1 | H | Cz12 | m-D1 |
| 2566 | m-D1 | m-D1 | m-D1 | m-D1 | Cz12 |
| 2567 | m-D1 | m-D1 | m-D1 | Cz12 | m-D1 |
| 2568 | m-D1 | m-D1 | Cz12 | m-D1 | m-D1 |
| 2569 | m-D2 | Cz12 | Cz12 | Cz12 | Cz12 |
| 2570 | Cz12 | m-D2 | Cz12 | Cz12 | Cz12 |
| 2571 | Cz12 | Cz12 | m-D2 | Cz12 | Cz12 |
| 2572 | m-D2 | Cz12 | Cz12 | Cz12 | H |
| 2573 | Cz12 | m-D2 | Cz12 | Cz12 | H |
| 2574 | Cz12 | Cz12 | m-D2 | Cz12 | H |
| 2575 | Cz12 | Cz12 | Cz12 | m-D2 | H |
| 2576 | m-D2 | Cz12 | Cz12 | H | Cz12 |
| 2577 | Cz12 | m-D2 | Cz12 | H | Cz12 |
| 2578 | Cz12 | Cz12 | m-D2 | H | Cz12 |
| 2579 | Cz12 | Cz12 | Cz12 | H | m-D2 |
| 2580 | m-D2 | Cz12 | H | Cz12 | Cz12 |
| 2581 | Cz12 | m-D2 | H | Cz12 | Cz12 |
| 2582 | m-D2 | Cz12 | Cz12 | H | H |
| 2583 | Cz12 | m-D2 | Cz12 | H | H |
| 2584 | Cz12 | Cz12 | m-D2 | H | H |
| 2585 | m-D2 | Cz12 | H | Cz12 | H |
| 2586 | Cz12 | m-D2 | H | Cz12 | H |
| 2587 | Cz12 | Cz12 | H | m-D2 | H |
| 2588 | m-D2 | H | Cz12 | Cz12 | H |
| 2589 | Cz12 | H | m-D2 | Cz12 | H |
| 2590 | Cz12 | H | Cz12 | m-D2 | H |
| 2591 | H | m-D2 | Cz12 | Cz12 | H |
| 2592 | H | Cz12 | m-D2 | Cz12 | H |
| 2593 | m-D2 | Cz12 | H | H | Cz12 |
| 2594 | Cz12 | m-D2 | H | H | Cz12 |
| 2595 | Cz12 | Cz12 | H | H | m-D2 |
| 2596 | m-D2 | H | Cz12 | H | Cz12 |
| 2597 | Cz12 | H | m-D2 | H | Cz12 |
| 2598 | m-D2 | Cz12 | H | H | H |
| 2599 | Cz12 | m-D2 | H | H | H |
| 2600 | m-D2 | H | Cz12 | H | H |
| 2601 | Cz12 | H | m-D2 | H | H |
| 2602 | H | m-D2 | Cz12 | H | H |
| 2603 | H | Cz12 | m-D2 | H | H |
| 2604 | m-D2 | H | H | Cz12 | H |
| 2605 | Cz12 | H | H | m-D2 | H |
| 2606 | H | m-D2 | H | Cz12 | H |
| 2607 | m-D2 | H | H | H | Cz12 |
| 2608 | m-D2 | m-D2 | Cz12 | Cz12 | H |
| 2609 | m-D2 | Cz12 | m-D2 | Cz12 | H |
| 2610 | m-D2 | Cz12 | Cz12 | m-D2 | H |
| 2611 | Cz12 | m-D2 | m-D2 | Cz12 | H |
| 2612 | Cz12 | m-D2 | Cz12 | m-D2 | H |
| 2613 | Cz12 | Cz12 | m-D2 | m-D2 | H |
| 2614 | m-D2 | m-D2 | Cz12 | H | Cz12 |
| 2615 | m-D2 | Cz12 | m-D2 | H | Cz12 |
| 2616 | m-D2 | Cz12 | Cz12 | H | m-D2 |
| 2617 | Cz12 | m-D2 | m-D2 | H | Cz12 |
| 2618 | Cz12 | m-D2 | Cz12 | H | m-D2 |
| 2619 | Cz12 | Cz12 | m-D2 | H | m-D2 |
| 2620 | m-D2 | m-D2 | H | Cz12 | Cz12 |
| 2621 | m-D2 | Cz12 | H | m-D2 | Cz12 |
| 2622 | m-D2 | Cz12 | H | Cz12 | m-D2 |
| 2623 | Cz12 | m-D2 | H | m-D2 | Cz12 |
| 2624 | m-D2 | m-D2 | Cz12 | H | H |
| 2625 | m-D2 | Cz12 | m-D2 | H | H |
| 2626 | Cz12 | m-D2 | m-D2 | H | H |
| 2627 | m-D2 | m-D2 | H | Cz12 | H |
| 2628 | m-D2 | Cz12 | H | m-D2 | H |
| 2629 | Cz12 | m-D2 | H | m-D2 | H |
| 2630 | m-D2 | H | m-D2 | Cz12 | H |
| 2631 | m-D2 | H | Cz12 | m-D2 | H |
| 2632 | Cz12 | H | m-D2 | m-D2 | H |
| 2633 | H | m-D2 | m-D2 | Cz12 | H |
| 2634 | H | m-D2 | Cz12 | m-D2 | H |
| 2635 | m-D2 | m-D2 | H | H | Cz12 |
| 2636 | m-D2 | Cz12 | H | H | m-D2 |
| 2637 | Cz12 | m-D2 | H | H | m-D2 |
| 2638 | m-D2 | H | m-D2 | H | Cz12 |
| 2639 | m-D2 | H | Cz12 | H | m-D2 |
| 2640 | m-D2 | m-D2 | m-D2 | Cz12 | Cz12 |
| 2641 | m-D2 | m-D2 | Cz12 | m-D2 | Cz12 |
| 2642 | m-D2 | m-D2 | Cz12 | Cz12 | m-D2 |
| 2643 | m-D2 | Cz12 | m-D2 | m-D2 | Cz12 |
| 2644 | m-D2 | Cz12 | m-D2 | Cz12 | m-D2 |
| 2645 | m-D2 | m-D2 | m-D2 | Cz12 | H |
| 2646 | m-D2 | m-D2 | Cz12 | m-D2 | H |
| 2647 | m-D2 | Cz12 | m-D2 | Cz12 | H |
| 2648 | Cz12 | m-D2 | m-D2 | m-D2 | H |
| 2649 | m-D2 | m-D2 | m-D2 | H | Cz12 |
| 2650 | m-D2 | m-D2 | Cz12 | H | m-D2 |
| 2651 | m-D2 | Cz12 | m-D2 | H | m-D2 |
| 2652 | Cz12 | m-D2 | m-D2 | H | m-D2 |
| 2653 | m-D2 | m-D2 | H | m-D2 | Cz12 |
| 2654 | m-D2 | m-D2 | H | Cz12 | m-D2 |
| 2655 | m-D2 | m-D2 | m-D2 | m-D2 | Cz12 |
| 2656 | m-D2 | m-D2 | m-D2 | Cz12 | m-D2 |
| 2657 | m-D2 | m-D2 | Cz12 | m-D2 | m-D2 |
| 2658 | m-D3 | Cz12 | Cz12 | Cz12 | Cz12 |
| 2659 | Cz12 | m-D3 | Cz12 | Cz12 | Cz12 |
| 2660 | Cz12 | Cz12 | m-D3 | Cz12 | Cz12 |
| 2661 | m-D3 | Cz12 | Cz12 | Cz12 | H |
| 2662 | Cz12 | m-D3 | Cz12 | Cz12 | H |
| 2663 | Cz12 | Cz12 | m-D3 | Cz12 | H |
| 2664 | Cz12 | Cz12 | Cz12 | m-D3 | H |
| 2665 | m-D3 | Cz12 | Cz12 | H | Cz12 |
| 2666 | Cz12 | m-D3 | Cz12 | H | Cz12 |
| 2667 | Cz12 | Cz12 | m-D3 | H | Cz12 |
| 2668 | Cz12 | Cz12 | Cz12 | H | m-D3 |
| 2669 | m-D3 | Cz12 | H | Cz12 | Cz12 |
| 2670 | Cz12 | m-D3 | H | Cz12 | Cz12 |
| 2671 | m-D3 | Cz12 | Cz12 | H | H |
| 2672 | Cz12 | m-D3 | Cz12 | H | H |
| 2673 | Cz12 | Cz12 | m-D3 | H | H |
| 2674 | m-D3 | Cz12 | H | Cz12 | H |
| 2675 | Cz12 | m-D3 | H | Cz12 | H |
| 2676 | Cz12 | Cz12 | H | m-D3 | H |
| 2677 | m-D3 | H | Cz12 | Cz12 | H |
| 2678 | Cz12 | H | m-D3 | Cz12 | H |
| 2679 | Cz12 | H | Cz12 | m-D3 | H |
| 2680 | H | m-D3 | Cz12 | Cz12 | H |
| 2681 | H | Cz12 | m-D3 | Cz12 | H |
| 2682 | m-D3 | Cz12 | H | H | Cz12 |
| 2683 | Cz12 | m-D3 | H | H | Cz12 |
| 2684 | Cz12 | Cz12 | H | H | m-D3 |
| 2685 | m-D3 | H | Cz12 | H | Cz12 |
| 2686 | Cz12 | H | m-D3 | H | Cz12 |
| 2687 | m-D3 | Cz12 | H | H | H |
| 2688 | Cz12 | m-D3 | H | H | H |
| 2689 | m-D3 | H | Cz12 | H | H |
| 2690 | Cz12 | H | m-D3 | H | H |
| 2691 | H | m-D3 | Cz12 | H | H |
| 2692 | H | Cz12 | m-D3 | H | H |
| 2693 | m-D3 | H | H | Cz12 | H |
| 2694 | Cz12 | H | H | m-D3 | H |
| 2695 | H | m-D3 | H | Cz12 | H |
| 2696 | m-D3 | H | H | H | Cz12 |
| 2697 | m-D3 | m-D3 | Cz12 | Cz12 | H |
| 2698 | m-D3 | Cz12 | m-D3 | Cz12 | H |
| 2699 | m-D3 | Cz12 | Cz12 | m-D3 | H |
| 2700 | Cz12 | m-D3 | m-D3 | Cz12 | H |
| 2701 | Cz12 | m-D3 | Cz12 | m-D3 | H |
| 2702 | Cz12 | Cz12 | m-D3 | m-D3 | H |
| 2703 | m-D3 | m-D3 | Cz12 | H | Cz12 |
| 2704 | m-D3 | Cz12 | m-D3 | H | Cz12 |
| 2705 | m-D3 | Cz12 | Cz12 | H | m-D3 |
| 2706 | Cz12 | m-D3 | m-D3 | H | Cz12 |
| 2707 | Cz12 | m-D3 | Cz12 | H | m-D3 |
| 2708 | Cz12 | Cz12 | m-D3 | H | m-D3 |
| 2709 | m-D3 | m-D3 | H | Cz12 | Cz12 |
| 2710 | m-D3 | Cz12 | H | m-D3 | Cz12 |
| 2711 | m-D3 | Cz12 | H | Cz12 | m-D3 |
| 2712 | Cz12 | m-D3 | H | m-D3 | Cz12 |
| 2713 | m-D3 | m-D3 | Cz12 | H | H |
| 2714 | m-D3 | Cz12 | m-D3 | H | H |
| 2715 | Cz12 | m-D3 | m-D3 | H | H |
| 2716 | m-D3 | m-D3 | H | Cz12 | H |
| 2717 | m-D3 | Cz12 | H | m-D3 | H |
| 2718 | Cz12 | m-D3 | H | m-D3 | H |
| 2719 | m-D3 | H | m-D3 | Cz12 | H |
| 2720 | m-D3 | H | Cz12 | m-D3 | H |
| 2721 | Cz12 | H | m-D3 | m-D3 | H |
| 2722 | H | m-D3 | m-D3 | Cz12 | H |
| 2723 | H | m-D3 | Cz12 | m-D3 | H |
| 2724 | m-D3 | m-D3 | H | H | Cz12 |
| 2725 | m-D3 | Cz12 | H | H | m-D3 |
| 2726 | Cz12 | m-D3 | H | H | m-D3 |
| 2727 | m-D3 | H | m-D3 | H | Cz12 |
| 2728 | m-D3 | H | Cz12 | H | m-D3 |
| 2729 | m-D3 | m-D3 | m-D3 | Cz12 | Cz12 |
| 2730 | m-D3 | m-D3 | Cz12 | m-D3 | Cz12 |
| 2731 | m-D3 | m-D3 | Cz12 | Cz12 | m-D3 |
| 2732 | m-D3 | Cz12 | m-D3 | m-D3 | Cz12 |
| 2733 | m-D3 | Cz12 | m-D3 | Cz12 | m-D3 |
| 2734 | m-D3 | m-D3 | m-D3 | Cz12 | H |
| 2735 | m-D3 | m-D3 | Cz12 | m-D3 | H |
| 2736 | m-D3 | Cz12 | m-D3 | Cz12 | H |
| 2737 | Cz12 | m-D3 | m-D3 | m-D3 | H |
| 2738 | m-D3 | m-D3 | m-D3 | H | Cz12 |
| 2739 | m-D3 | m-D3 | Cz12 | H | m-D3 |
| 2740 | m-D3 | Cz12 | m-D3 | H | m-D3 |
| 2741 | Cz12 | m-D3 | m-D3 | H | m-D3 |
| 2742 | m-D3 | m-D3 | H | m-D3 | Cz12 |
| 2743 | m-D3 | m-D3 | H | Cz12 | m-D3 |
| 2744 | m-D3 | m-D3 | m-D3 | m-D3 | Cz12 |
| 2745 | m-D3 | m-D3 | m-D3 | Cz12 | m-D3 |
| 2746 | m-D3 | m-D3 | Cz12 | m-D3 | m-D3 |
| 2747 | F | Cz | m-D1 | Cz | Cz |
| 2748 | Cz | F | m-D1 | Cz | Cz |
| 2749 | Cz | F | m-D1 | F | Cz |
| 2750 | Cz | F | m-D1 | F | F |
| 2751 | Cz | F | m-D1 | Cz | H |
| 2752 | Cz | F | m-D1 | F | H |
| 2753 | F | Cz | m-D1 | Cz | H |
| 2754 | F | F | m-D1 | Cz | H |
| 2755 | F | Cz | m-D1 | H | Cz |
| 2756 | Cz | F | m-D1 | H | Cz |
| 2757 | F | F | m-D1 | H | Cz |
| 2758 | F | Cz | m-D1 | H | F |
| 2759 | F | H | m-D1 | H | Cz |
| 2760 | F | Cz | m-D2 | Cz | Cz |
| 2761 | Cz | F | m-D2 | Cz | Cz |
| 2762 | Cz | F | m-D2 | F | Cz |
| 2763 | Cz | F | m-D2 | F | F |
| 2764 | Cz | F | m-D2 | Cz | H |
| 2765 | Cz | F | m-D2 | F | H |
| 2766 | F | Cz | m-D2 | Cz | H |
| 2767 | F | F | m-D2 | Cz | H |
| 2768 | F | Cz | m-D2 | H | Cz |
| 2769 | Cz | F | m-D2 | H | Cz |
| 2770 | F | F | m-D2 | H | Cz |
| 2771 | F | Cz | m-D2 | H | F |
| 2772 | F | H | m-D2 | H | Cz |
| 2773 | F | Cz | m-D3 | Cz | Cz |
| 2774 | Cz | F | m-D3 | Cz | Cz |
| 2775 | Cz | F | m-D3 | F | Cz |
| 2776 | Cz | F | m-D3 | F | F |
| 2777 | Cz | F | m-D3 | Cz | H |
| 2778 | Cz | F | m-D3 | F | H |
| 2779 | F | Cz | m-D3 | Cz | H |
| 2780 | F | F | m-D3 | Cz | H |
| 2781 | F | Cz | m-D3 | H | Cz |
| 2782 | Cz | F | m-D3 | H | Cz |
| 2783 | F | F | m-D3 | H | Cz |
| 2784 | F | Cz | m-D3 | H | F |
| 2785 | F | H | m-D3 | H | Cz |

Next, compounds represented by the following general formula (C) are described.

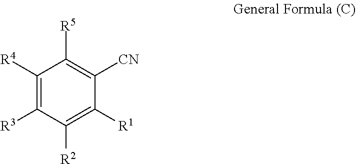

In the general formula (C), 3 or more of R¹, R², R⁴ and R⁵ each independently represent a substituted or unsubstituted 9-carbazolyl group, a substituted or unsubstituted 10-phenoxazyl group, a substituted or unsubstituted 10-phenothiazyl group, or a cyano group. The remaining substituents each represent a hydrogen atom or a substituent, but the substituent is not a substituted or unsubstituted 9-carbazolyl group, a substituted or unsubstituted 10-phenoxazyl group, or a substituted or unsubstituted 10-phenothiazyl group. One or more carbon atoms constituting each ring skeleton of the 9-carbazolyl group, the 10-phenoxazyl group and the 10-phenothiazyl group may be substituted with a nitrogen atom. R³ each independently represents a hydrogen atom or a substituent, but the substituent is not a substituted or unsubstituted 9-carbazolyl group, a substituted or unsubstituted 10-phenoxazyl group, a cyano group, a substituted or unsubstituted 10-phenothiazyl group, a substituted or unsubstituted aryl group, a substituted or unsubstituted heteroaryl group or a substituted or unsubstituted alkynyl group.

Specific examples (D1 to D42) of R¹, R², R⁴ and R⁵ in the general formula (C) are shown below.

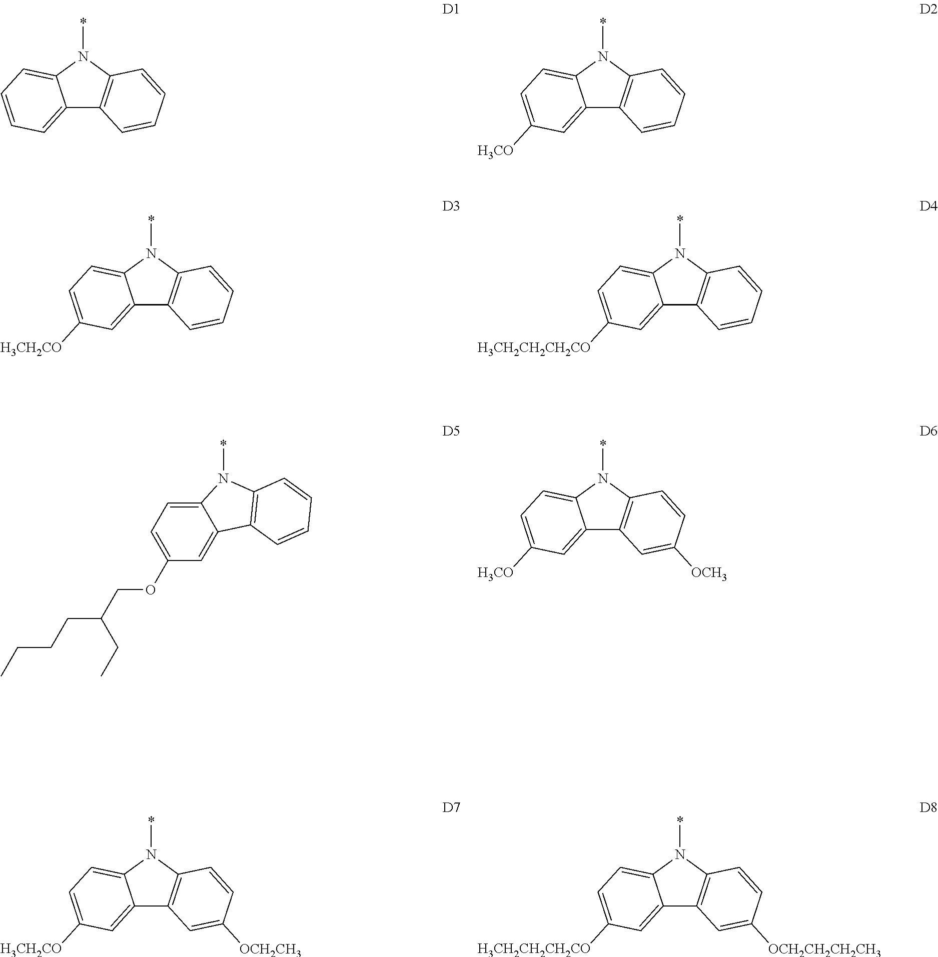

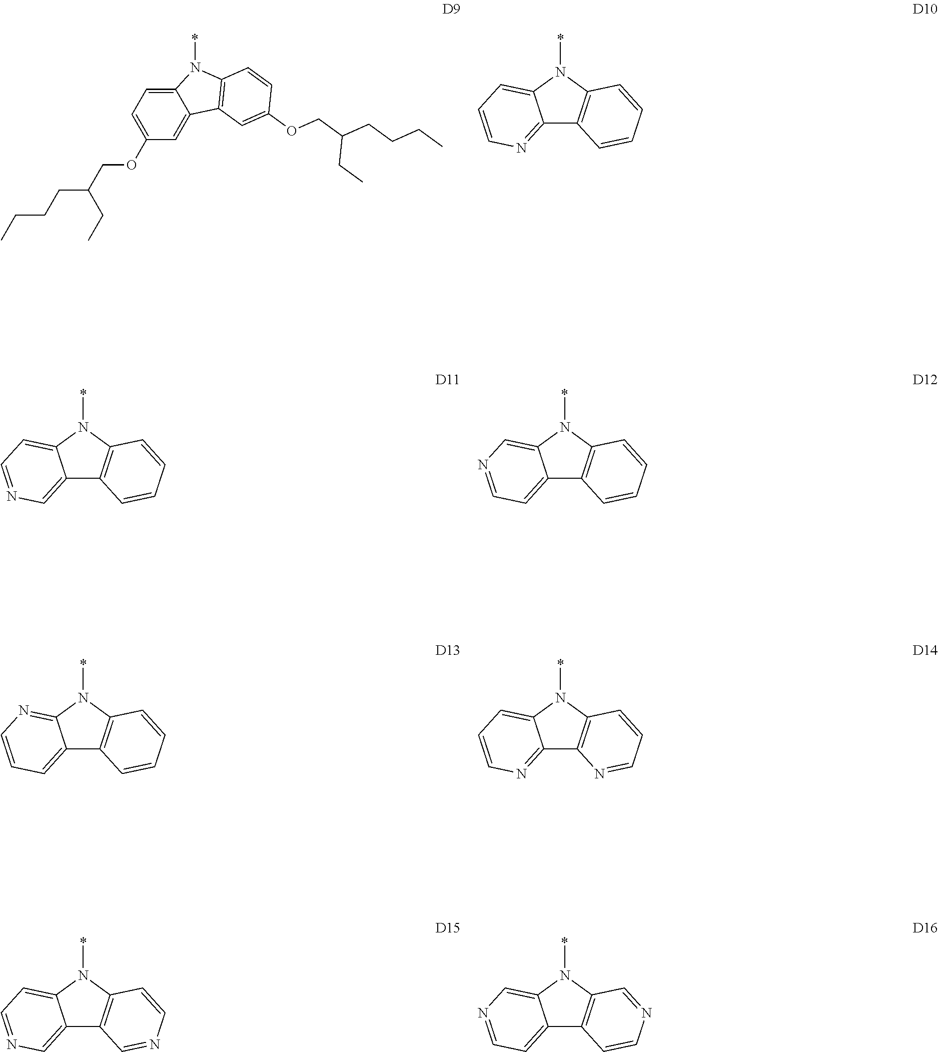

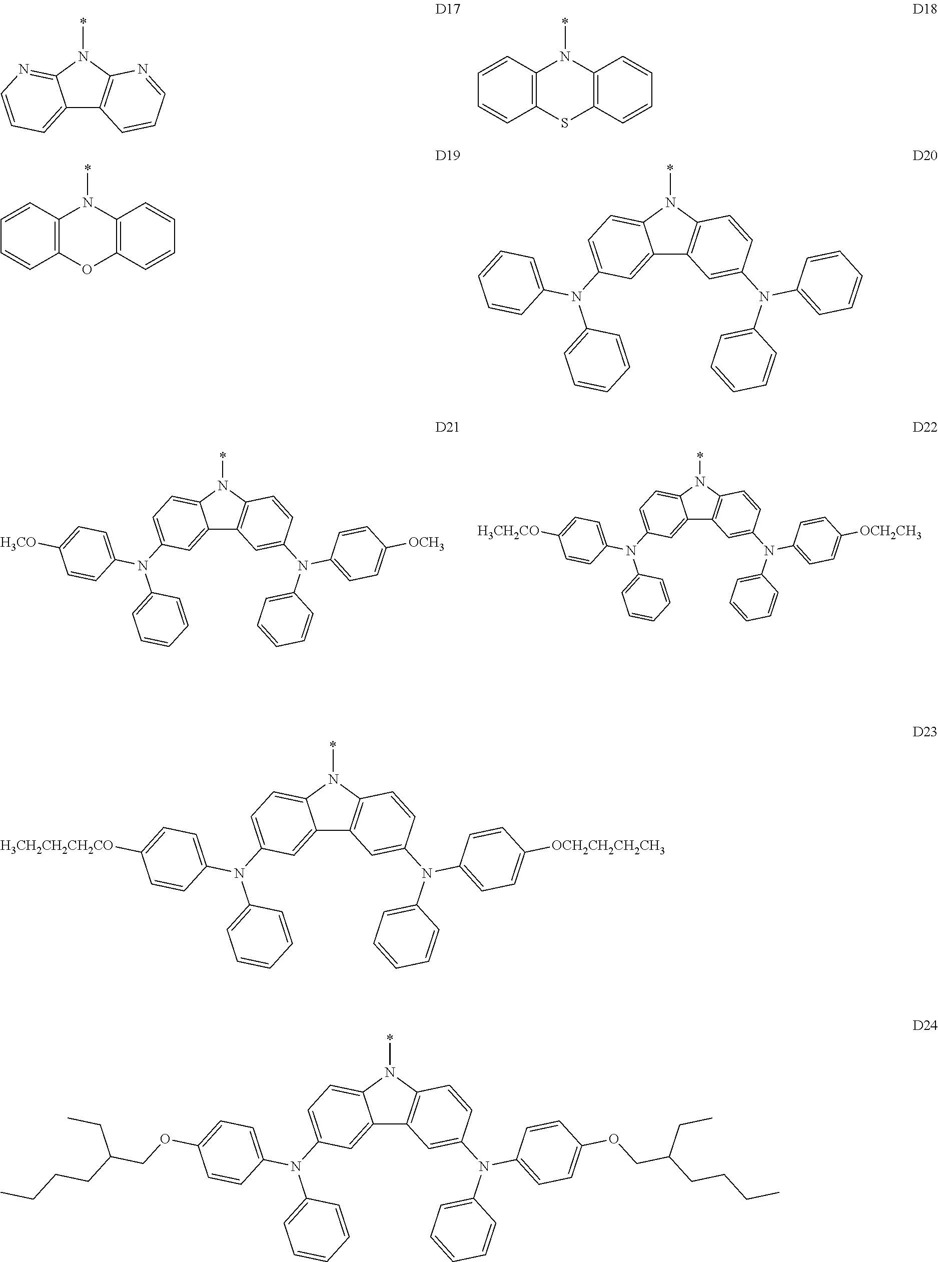

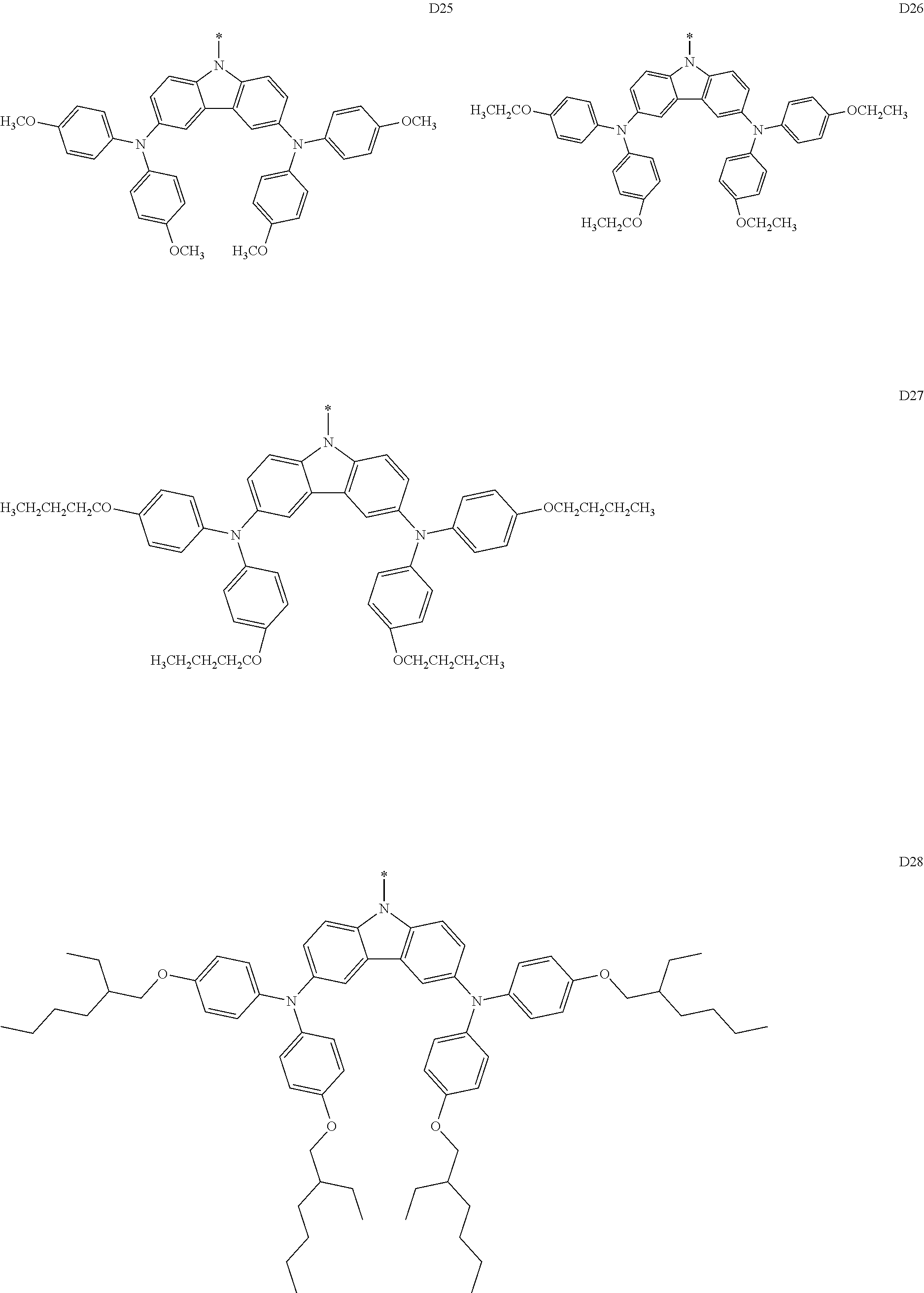

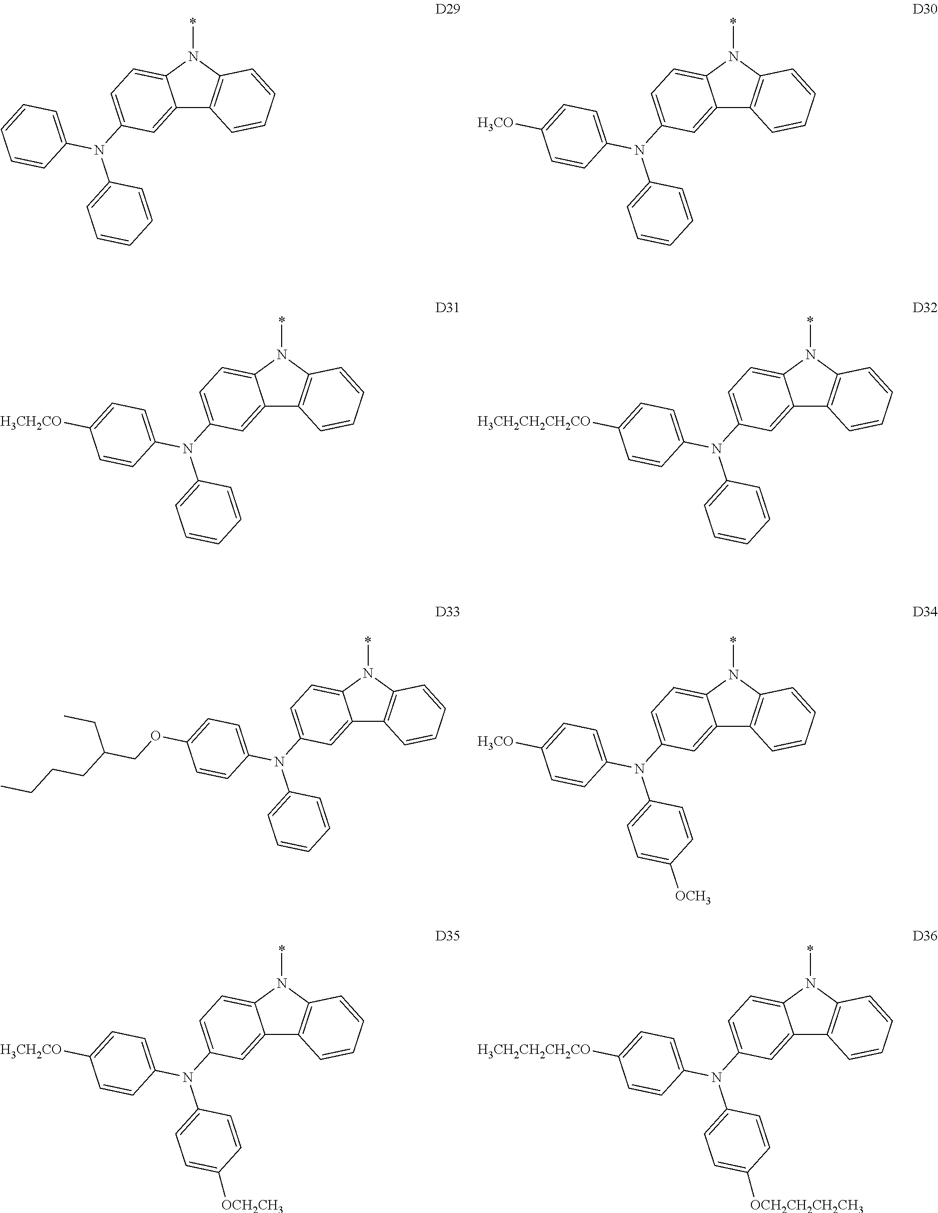

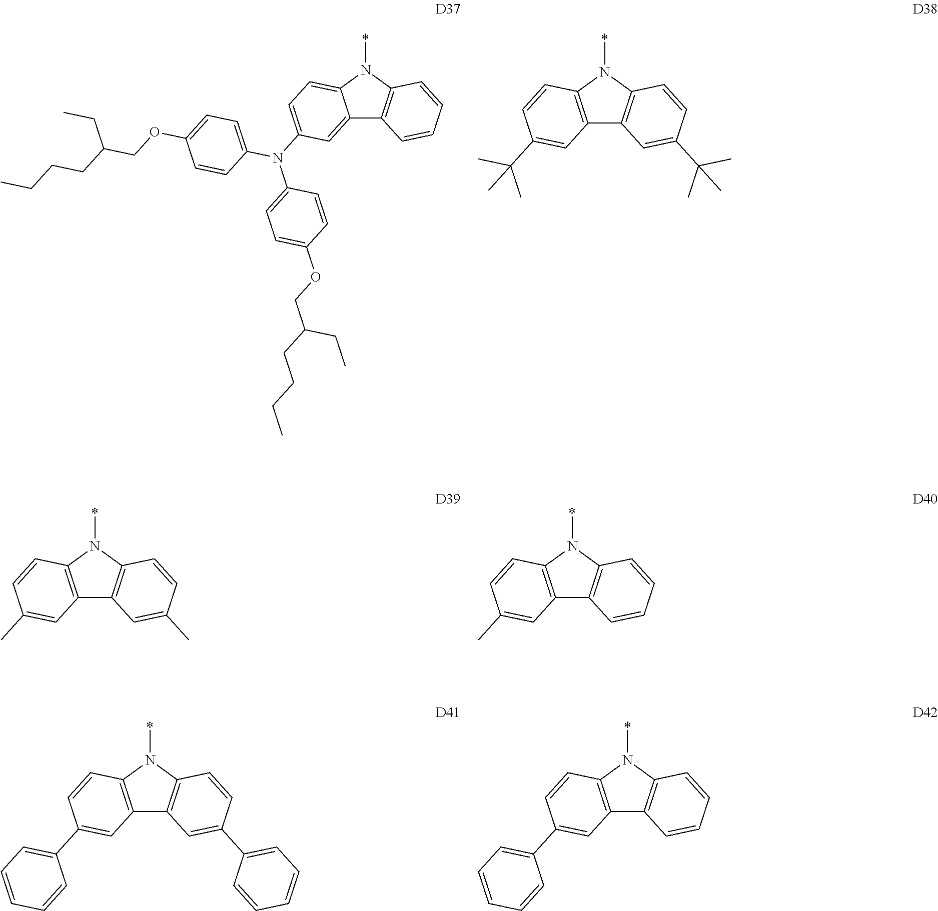

Specific examples of the compounds represented by the general formula (C) are shown below.

TABLE 10

| Compound No. | R¹ | R² | R³ | R⁴ | R⁵ |
|---|---|---|---|---|---|
| 1 | D1 | D1 | H | D1 | D1 |
| 2 | D1 | D1 | H | H | D1 |
| 3 | D1 | D1 | H | D1 | H |
| 4 | D2 | D2 | H | D2 | H |
| 5 | D2 | D2 | H | H | D2 |
| 6 | D2 | D2 | H | D2 | D2 |
| 7 | D3 | D3 | H | D3 | H |
| 8 | D3 | D3 | H | H | D3 |
| 9 | D3 | D3 | H | D3 | D3 |
| 10 | D4 | D4 | H | D4 | H |
| 11 | D4 | D4 | H | H | D4 |
| 12 | D4 | D4 | H | D4 | D4 |
| 13 | D5 | D5 | H | D5 | H |
| 14 | D5 | D5 | H | H | D5 |
| 15 | D5 | D5 | H | D5 | D5 |
| 16 | D6 | D6 | H | D6 | H |
| 17 | D6 | D6 | H | H | D6 |
| 18 | D6 | D6 | H | D6 | D6 |
| 19 | D7 | D7 | H | D7 | H |
| 20 | D7 | D7 | H | H | D7 |
| 21 | D7 | D7 | H | D7 | D7 |
| 22 | D8 | D8 | H | D8 | H |
| 23 | D8 | D8 | H | H | D8 |
| 24 | D8 | D8 | H | D8 | D8 |
| 25 | D9 | D9 | H | D9 | H |
| 26 | D9 | D9 | H | H | D9 |
| 27 | D9 | D9 | H | D9 | D9 |
| 28 | D10 | D10 | H | D10 | H |
| 29 | D10 | D10 | H | H | D10 |
| 30 | D10 | D10 | H | D10 | D10 |
| 31 | D11 | D11 | H | D11 | H |
| 32 | D11 | D11 | H | H | D11 |
| 33 | D11 | D11 | H | D11 | D11 |
| 34 | D12 | D12 | H | D12 | H |
| 35 | D12 | D12 | H | H | D12 |
| 36 | D12 | D12 | H | D12 | D12 |
| 37 | D13 | D13 | H | D13 | H |
| 38 | D13 | D13 | H | H | D13 |
| 39 | D13 | D13 | H | D13 | D13 |
| 40 | D14 | D14 | H | D14 | H |
| 41 | D14 | D14 | H | H | D14 |
| 42 | D14 | D14 | H | D14 | D14 |
| 43 | D15 | D15 | H | D15 | H |
| 44 | D15 | D15 | H | H | D15 |
| 45 | D15 | D15 | H | D15 | D15 |
| 46 | D16 | D16 | H | D16 | H |
| 47 | D16 | D16 | H | H | D16 |
| 48 | D16 | D16 | H | D16 | D16 |
| 49 | D17 | D17 | H | D17 | H |
| 50 | D17 | D17 | H | H | D17 |
| 51 | D17 | D17 | H | D17 | D17 |
| 52 | D18 | D18 | H | D18 | H |
| 53 | D18 | D18 | H | H | D18 |
| 54 | D18 | D18 | H | D18 | D18 |
| 55 | D19 | D19 | H | D19 | H |
| 56 | D19 | D19 | H | H | D19 |
| 57 | D19 | D19 | H | D19 | D19 |
| 58 | D20 | D20 | H | D20 | H |
| 59 | D20 | D20 | H | H | D20 |
| 60 | D20 | D20 | H | D20 | D20 |
| 61 | D21 | D21 | H | D21 | H |
| 62 | D21 | D21 | H | H | D21 |
| 63 | D21 | D21 | H | D21 | D21 |
| 64 | D22 | D22 | H | D22 | H |
| 65 | D22 | D22 | H | H | D22 |
| 66 | D22 | D22 | H | D22 | D22 |
| 67 | D23 | D23 | H | D23 | H |
| 68 | D23 | D23 | H | H | D23 |
| 69 | D23 | D23 | H | D23 | D23 |
| 70 | D24 | D24 | H | D24 | H |
| 71 | D24 | D24 | H | H | D24 |
| 72 | D24 | D24 | H | D24 | D24 |
| 73 | D25 | D25 | H | D25 | H |
| 74 | D25 | D25 | H | H | D25 |
| 75 | D25 | D25 | H | D25 | D25 |
| 76 | D26 | D26 | H | D26 | H |
| 77 | D26 | D26 | H | H | D26 |
| 78 | D26 | D26 | H | D26 | D26 |
| 79 | D27 | D27 | H | D27 | H |
| 80 | D27 | D27 | H | H | D27 |
| 81 | D27 | D27 | H | D27 | D27 |
| 82 | D28 | D28 | H | D28 | H |
| 83 | D28 | D28 | H | H | D28 |
| 84 | D28 | D28 | H | D28 | D28 |
| 85 | D29 | D29 | H | D29 | H |
| 86 | D29 | D29 | H | H | D29 |
| 87 | D29 | D29 | H | D29 | D29 |
| 88 | D30 | D30 | H | D30 | H |
| 89 | D30 | D30 | H | H | D30 |
| 90 | D30 | D30 | H | D30 | D30 |
| 91 | D31 | D31 | H | D31 | H |
| 92 | D31 | D31 | H | H | D31 |
| 93 | D31 | D31 | H | D31 | D31 |
| 94 | D32 | D32 | H | D32 | H |
| 95 | D32 | D32 | H | H | D32 |
| 96 | D32 | D32 | H | D32 | D32 |
| 97 | D33 | D33 | H | D33 | H |
| 98 | D33 | D33 | H | H | D33 |
| 99 | D33 | D33 | H | D33 | D33 |
| 100 | D34 | D34 | H | D34 | H |
| 101 | D34 | D34 | H | H | D34 |
| 102 | D34 | D34 | H | D34 | D34 |
| 103 | D35 | D35 | H | D35 | H |
| 104 | D35 | D35 | H | H | D35 |
| 105 | D35 | D35 | H | D35 | D35 |
| 106 | D36 | D36 | H | D36 | H |
| 107 | D36 | D36 | H | H | D36 |
| 108 | D36 | D36 | H | D36 | D36 |
| 109 | D37 | D37 | H | D37 | H |
| 110 | D37 | D37 | H | H | D37 |
| 111 | D1 | D1 | methyl | D1 | methyl |
| 112 | D1 | D1 | methyl | methyl | D1 |
| 113 | D1 | D1 | methyl | D1 | D1 |
| 114 | D2 | D2 | methyl | D2 | methyl |
| 115 | D2 | D2 | methyl | methyl | D2 |
| 116 | D2 | D2 | methyl | D2 | D2 |
| 117 | D3 | D3 | methyl | D3 | methyl |
| 118 | D3 | D3 | methyl | methyl | D3 |
| 119 | D3 | D3 | methyl | D3 | D3 |
| 120 | D4 | D4 | methyl | D4 | methyl |
| 121 | D4 | D4 | methyl | methyl | D4 |
| 122 | D4 | D4 | methyl | D4 | D4 |
| 123 | D5 | D5 | methyl | D5 | methyl |
| 124 | D5 | D5 | methyl | methyl | D5 |
| 125 | D5 | D5 | methyl | D5 | D5 |
| 126 | D6 | D6 | methyl | D6 | methyl |
| 127 | D6 | D6 | methyl | methyl | D6 |
| 128 | D6 | D6 | methyl | D6 | D6 |
| 129 | D7 | D7 | methyl | D7 | methyl |
| 130 | D7 | D7 | methyl | methyl | D7 |
| 131 | D7 | D7 | methyl | D7 | D7 |
| 132 | D8 | D8 | methyl | D8 | methyl |
| 133 | D8 | D8 | methyl | methyl | D8 |
| 134 | D8 | D8 | methyl | D8 | D8 |
| 135 | D9 | D9 | methyl | D9 | methyl |
| 136 | D9 | D9 | methyl | methyl | D9 |
| 137 | D9 | D9 | methyl | D9 | D9 |
| 138 | D10 | D10 | methyl | D10 | methyl |
| 139 | D10 | D10 | methyl | methyl | D10 |
| 140 | D10 | D10 | methyl | D10 | D10 |
| 141 | D11 | D11 | methyl | D11 | methyl |
| 142 | D11 | D11 | methyl | methyl | D11 |
| 143 | D11 | D11 | methyl | D11 | D11 |
| 144 | D12 | D12 | methyl | D12 | methyl |
| 145 | D12 | D12 | methyl | methyl | D12 |
| 146 | D12 | D12 | methyl | D12 | D12 |
| 147 | D13 | D13 | methyl | D13 | methyl |
| 148 | D13 | D13 | methyl | methyl | D13 |
| 149 | D13 | D13 | methyl | D13 | D13 |
| 150 | D14 | D14 | methyl | D14 | methyl |
| 151 | D14 | D14 | methyl | methyl | D14 |
| 152 | D14 | D14 | methyl | D14 | D14 |
| 153 | D15 | D15 | methyl | D15 | methyl |
| 154 | D15 | D15 | methyl | methyl | D15 |
| 155 | D15 | D15 | methyl | D15 | D15 |
| 156 | D16 | D16 | methyl | D16 | methyl |
| 157 | D16 | D16 | methyl | methyl | D16 |
| 158 | D16 | D16 | methyl | D16 | D16 |
| 159 | D17 | D17 | methyl | D17 | methyl |
| 160 | D17 | D17 | methyl | methyl | D17 |
| 161 | D17 | D17 | methyl | D17 | D17 |
| 162 | D18 | D18 | methyl | D18 | methyl |
| 163 | D18 | D18 | methyl | methyl | D18 |
| 164 | D18 | D18 | methyl | D18 | D18 |
| 165 | D19 | D19 | methyl | D19 | methyl |
| 166 | D19 | D19 | methyl | methyl | D19 |
| 167 | D19 | D19 | methyl | D19 | D19 |
| 168 | D20 | D20 | methyl | D20 | methyl |
| 169 | D20 | D20 | methyl | methyl | D20 |
| 170 | D20 | D20 | methyl | D20 | D20 |
| 171 | D21 | D21 | methyl | D21 | methyl |
| 172 | D21 | D21 | methyl | methyl | D21 |
| 173 | D21 | D21 | methyl | D21 | D21 |
| 174 | D22 | D22 | methyl | D22 | methyl |
| 175 | D22 | D22 | methyl | methyl | D22 |
| 176 | D22 | D22 | methyl | D22 | D22 |
| 177 | D23 | D23 | methyl | D23 | methyl |
| 178 | D23 | D23 | methyl | methyl | D23 |
| 179 | D23 | D23 | methyl | D23 | D23 |
| 180 | D24 | D24 | methyl | D24 | methyl |
| 181 | D24 | D24 | methyl | methyl | D24 |
| 182 | D24 | D24 | methyl | D24 | D24 |
| 183 | D25 | D25 | methyl | D25 | methyl |
| 184 | D25 | D25 | methyl | methyl | D25 |
| 185 | D25 | D25 | methyl | D25 | D25 |
| 186 | D26 | D26 | methyl | D26 | methyl |
| 187 | D26 | D26 | methyl | methyl | D26 |
| 188 | D26 | D26 | methyl | D26 | D26 |
| 189 | D27 | D27 | methyl | D27 | methyl |
| 190 | D27 | D27 | methyl | methyl | D27 |
| 191 | D27 | D27 | methyl | D27 | D27 |
| 192 | D28 | D28 | methyl | D28 | methyl |
| 193 | D28 | D28 | methyl | methyl | D28 |
| 194 | D28 | D28 | methyl | D28 | D28 |
| 195 | D29 | D29 | methyl | D29 | methyl |
| 196 | D29 | D29 | methyl | methyl | D29 |
| 197 | D29 | D29 | methyl | D29 | D29 |
| 198 | D30 | D30 | methyl | D30 | methyl |
| 199 | D30 | D30 | methyl | methyl | D30 |
| 200 | D30 | D30 | methyl | D30 | D30 |
| 201 | D31 | D31 | methyl | D31 | methyl |
| 202 | D31 | D31 | methyl | methyl | D31 |
| 203 | D31 | D31 | methyl | D31 | D31 |
| 204 | D32 | D32 | methyl | D32 | methyl |
| 205 | D32 | D32 | methyl | methyl | D32 |
| 206 | D32 | D32 | methyl | D32 | D32 |
| 207 | D33 | D33 | methyl | D33 | methyl |
| 208 | D33 | D33 | methyl | methyl | D33 |
| 209 | D33 | D33 | methyl | D33 | D33 |
| 210 | D34 | D34 | methyl | D34 | methyl |
| 211 | D34 | D34 | methyl | methyl | D34 |
| 212 | D34 | D34 | methyl | D34 | D34 |
| 213 | D35 | D35 | methyl | D35 | methyl |
| 214 | D35 | D35 | methyl | methyl | D35 |
| 215 | D35 | D35 | methyl | D35 | D35 |
| 216 | D36 | D36 | methyl | D36 | methyl |
| 217 | D36 | D36 | methyl | methyl | D36 |
| 218 | D36 | D36 | methyl | D36 | D36 |
| 219 | D37 | D37 | methyl | D37 | methyl |
| 220 | D37 | D37 | methyl | methyl | D37 |
| 221 | D37 | D37 | methyl | D37 | D37 |
| 222 | D1 | D1 | isopropyl | D1 | isopropyl |
| 223 | D1 | D1 | isopropyl | isopropyl | D1 |
| 224 | D1 | D1 | isopropyl | D1 | D1 |
| 225 | D2 | D2 | isopropyl | D2 | isopropyl |
| 226 | D2 | D2 | isopropyl | isopropyl | D2 |
| 227 | D2 | D2 | isopropyl | D2 | D2 |
| 228 | D3 | D3 | isopropyl | D3 | isopropyl |
| 229 | D3 | D3 | isopropyl | isopropyl | D3 |
| 230 | D3 | D3 | isopropyl | D3 | D3 |
| 231 | D4 | D4 | isopropyl | D4 | isopropyl |
| 232 | D4 | D4 | isopropyl | isopropyl | D4 |
| 233 | D4 | D4 | isopropyl | D4 | D4 |
| 234 | D5 | D5 | isopropyl | D5 | isopropyl |
| 235 | D5 | D5 | isopropyl | isopropyl | D5 |
| 236 | D5 | D5 | isopropyl | D5 | D5 |
| 237 | D6 | D6 | isopropyl | D6 | isopropyl |
| 238 | D6 | D6 | isopropyl | isopropyl | D6 |
| 239 | D6 | D6 | isopropyl | D6 | D6 |
| 240 | D7 | D7 | isopropyl | D7 | isopropyl |
| 241 | D7 | D7 | isopropyl | isopropyl | D7 |
| 242 | D7 | D7 | isopropyl | D7 | D7 |
| 243 | D8 | D8 | isopropyl | D8 | isopropyl |
| 244 | D8 | D8 | isopropyl | isopropyl | D8 |
| 245 | D8 | D8 | isopropyl | D8 | D8 |
| 246 | D9 | D9 | isopropyl | D9 | isopropyl |
| 247 | D9 | D9 | isopropyl | isopropyl | D9 |
| 248 | D9 | D9 | isopropyl | D9 | D9 |
| 249 | D10 | D10 | isopropyl | D10 | isopropyl |
| 250 | D10 | D10 | isopropyl | isopropyl | D10 |
| 251 | D10 | D10 | isopropyl | D10 | D10 |
| 252 | D11 | D11 | isopropyl | D11 | isopropyl |
| 253 | D11 | D11 | isopropyl | isopropyl | D11 |
| 254 | D11 | D11 | isopropyl | D11 | D11 |
| 255 | D12 | D12 | isopropyl | D12 | isopropyl |
| 256 | D12 | D12 | isopropyl | isopropyl | D12 |
| 257 | D12 | D12 | isopropyl | D12 | D12 |
| 258 | D13 | D13 | isopropyl | D13 | isopropyl |
| 259 | D13 | D13 | isopropyl | isopropyl | D13 |
| 260 | D13 | D13 | isopropyl | D13 | D13 |
| 261 | D14 | D14 | isopropyl | D14 | isopropyl |
| 262 | D14 | D14 | isopropyl | isopropyl | D14 |
| 263 | D14 | D14 | isopropyl | D14 | D14 |
| 264 | D15 | D15 | isopropyl | D15 | isopropyl |
| 265 | D15 | D15 | isopropyl | isopropyl | D15 |
| 266 | D15 | D15 | isopropyl | D15 | D15 |
| 267 | D16 | D16 | isopropyl | D16 | isopropyl |
| 268 | D16 | D16 | isopropyl | isopropyl | D16 |
| 269 | D16 | D16 | isopropyl | D16 | D16 |
| 270 | D17 | D17 | isopropyl | D17 | isopropyl |
| 271 | D17 | D17 | isopropyl | isopropyl | D17 |
| 272 | D17 | D17 | isopropyl | D17 | D17 |
| 273 | D18 | D18 | isopropyl | D18 | isopropyl |
| 274 | D18 | D18 | isopropyl | isopropyl | D18 |
| 275 | D18 | D18 | isopropyl | D18 | D18 |
| 276 | D19 | D19 | isopropyl | D19 | isopropyl |
| 277 | D19 | D19 | isopropyl | isopropyl | D19 |
| 278 | D19 | D19 | isopropyl | D19 | D19 |
| 279 | D20 | D20 | isopropyl | D20 | isopropyl |
| 280 | D20 | D20 | isopropyl | isopropyl | D20 |
| 281 | D20 | D20 | isopropyl | D20 | D20 |
| 282 | D21 | D21 | isopropyl | D21 | isopropyl |
| 283 | D21 | D21 | isopropyl | isopropyl | D21 |
| 284 | D21 | D21 | isopropyl | D21 | D21 |
| 285 | D22 | D22 | isopropyl | D22 | isopropyl |
| 286 | D22 | D22 | isopropyl | isopropyl | D22 |
| 287 | D22 | D22 | isopropyl | D22 | D22 |
| 288 | D23 | D23 | isopropyl | D23 | isopropyl |
| 289 | D23 | D23 | isopropyl | isopropyl | D23 |
| 290 | D23 | D23 | isopropyl | D23 | D23 |
| 291 | D24 | D24 | isopropyl | D24 | isopropyl |
| 292 | D24 | D24 | isopropyl | isopropyl | D24 |
| 293 | D24 | D24 | isopropyl | D24 | D24 |
| 294 | D25 | D25 | isopropyl | D25 | isopropyl |
| 295 | D25 | D25 | isopropyl | isopropyl | D25 |
| 296 | D25 | D25 | isopropyl | D25 | D25 |
| 297 | D26 | D26 | isopropyl | D26 | isopropyl |
| 298 | D26 | D26 | isopropyl | isopropyl | D26 |
| 299 | D26 | D26 | isopropyl | D26 | D26 |
| 300 | D27 | D27 | isopropyl | D27 | isopropyl |
| 301 | D27 | D27 | isopropyl | isopropyl | D27 |
| 302 | D27 | D27 | isopropyl | D27 | D27 |
| 303 | D28 | D28 | isopropyl | D28 | isopropyl |
| 304 | D28 | D28 | isopropyl | isopropyl | D28 |
| 305 | D28 | D28 | isopropyl | D28 | D28 |
| 306 | D29 | D29 | isopropyl | D29 | isopropyl |
| 307 | D29 | D29 | isopropyl | isopropyl | D29 |
| 308 | D29 | D29 | isopropyl | D29 | D29 |
| 309 | D30 | D30 | isopropyl | D30 | isopropyl |
| 310 | D30 | D30 | isopropyl | isopropyl | D30 |
| 311 | D30 | D30 | isopropyl | D30 | D30 |
| 312 | D31 | D31 | isopropyl | D31 | isopropyl |
| 313 | D31 | D31 | isopropyl | isopropyl | D31 |
| 314 | D31 | D31 | isopropyl | D31 | D31 |
| 315 | D32 | D32 | isopropyl | D32 | isopropyl |
| 316 | D32 | D32 | isopropyl | isopropyl | D32 |
| 317 | D32 | D32 | isopropyl | D32 | D32 |
| 318 | D33 | D33 | isopropyl | D33 | isopropyl |
| 319 | D33 | D33 | isopropyl | isopropyl | D33 |
| 320 | D33 | D33 | isopropyl | D33 | D33 |
| 321 | D34 | D34 | isopropyl | D34 | isopropyl |
| 322 | D34 | D34 | isopropyl | isopropyl | D34 |
| 323 | D34 | D34 | isopropyl | D34 | D34 |
| 324 | D35 | D35 | isopropyl | D35 | isopropyl |
| 325 | D35 | D35 | isopropyl | isopropyl | D35 |
| 326 | D35 | D35 | isopropyl | D35 | D35 |
| 327 | D36 | D36 | isopropyl | D36 | isopropyl |
| 328 | D36 | D36 | isopropyl | isopropyl | D36 |
| 329 | D36 | D36 | isopropyl | D36 | D36 |
| 330 | D37 | D37 | isopropyl | D37 | isopropyl |
| 331 | D37 | D37 | isopropyl | isopropyl | D37 |
| 332 | D37 | D37 | isopropyl | D37 | D37 |
| 333 | D1 | D1 | tBu | D1 | tBu |
| 334 | D1 | D1 | tBu | tBu | D1 |
| 335 | D1 | D1 | tBu | D1 | D1 |
| 336 | D2 | D2 | tBu | D2 | tBu |
| 337 | D2 | D2 | tBu | tBu | D2 |
| 338 | D2 | D2 | tBu | D2 | D2 |
| 339 | D3 | D3 | tBu | D3 | tBu |
| 340 | D3 | D3 | tBu | tBu | D3 |
| 341 | D3 | D3 | tBu | D3 | D3 |
| 342 | D4 | D4 | tBu | D4 | tBu |
| 343 | D4 | D4 | tBu | tBu | D4 |
| 344 | D4 | D4 | tBu | D4 | D4 |
| 345 | D5 | D5 | tBu | D5 | tBu |
| 346 | D5 | D5 | tBu | tBu | D5 |
| 347 | D5 | D5 | tBu | D5 | D5 |
| 348 | D6 | D6 | tBu | D6 | tBu |
| 349 | D6 | D6 | tBu | tBu | D6 |
| 350 | D6 | D6 | tBu | D6 | D6 |
| 351 | D7 | D7 | tBu | D7 | tBu |
| 352 | D7 | D7 | tBu | tBu | D7 |
| 353 | D7 | D7 | tBu | D7 | D7 |
| 354 | D8 | D8 | tBu | D8 | tBu |
| 355 | D8 | D8 | tBu | tBu | D8 |
| 356 | D8 | D8 | tBu | D8 | D8 |
| 357 | D9 | D9 | tBu | D9 | tBu |
| 358 | D9 | D9 | tBu | tBu | D9 |
| 359 | D9 | D9 | tBu | D9 | D9 |
| 360 | D10 | D10 | tBu | D10 | tBu |
| 361 | D10 | D10 | tBu | tBu | D10 |
| 362 | D10 | D10 | tBu | D10 | D10 |
| 363 | D11 | D11 | tBu | D11 | tBu |
| 364 | D11 | D11 | tBu | tBu | D11 |
| 365 | D11 | D11 | tBu | D11 | D11 |
| 366 | D12 | D12 | tBu | D12 | tBu |
| 367 | D12 | D12 | tBu | tBu | D12 |
| 368 | D12 | D12 | tBu | D12 | D12 |
| 369 | D13 | D13 | tBu | D13 | tBu |
| 370 | D13 | D13 | tBu | tBu | D13 |
| 371 | D13 | D13 | tBu | D13 | D13 |
| 372 | D14 | D14 | tBu | D14 | tBu |
| 373 | D14 | D14 | tBu | tBu | D14 |
| 374 | D14 | D14 | tBu | D14 | D14 |
| 375 | D15 | D15 | tBu | D15 | tBu |
| 376 | D15 | D15 | tBu | tBu | D15 |
| 377 | D15 | D15 | tBu | D15 | D15 |
| 378 | D16 | D16 | tBu | D16 | tBu |
| 379 | D16 | D16 | tBu | tBu | D16 |
| 380 | D16 | D16 | tBu | D16 | D16 |
| 381 | D17 | D17 | tBu | D17 | tBu |
| 382 | D17 | D17 | tBu | tBu | D17 |
| 383 | D17 | D17 | tBu | D17 | D17 |
| 384 | D18 | D18 | tBu | D18 | tBu |
| 385 | D18 | D18 | tBu | tBu | D18 |
| 386 | D18 | D18 | tBu | D18 | D18 |
| 387 | D19 | D19 | tBu | D19 | tBu |
| 388 | D19 | D19 | tBu | tBu | D19 |
| 389 | D19 | D19 | tBu | D19 | D19 |
| 390 | D20 | D20 | tBu | D20 | tBu |
| 391 | D20 | D20 | tBu | tBu | D20 |
| 392 | D20 | D20 | tBu | D20 | D20 |
| 393 | D21 | D21 | tBu | D21 | tBu |
| 394 | D21 | D21 | tBu | tBu | D21 |
| 395 | D21 | D21 | tBu | D21 | D21 |
| 396 | D22 | D22 | tBu | D22 | tBu |
| 397 | D22 | D22 | tBu | tBu | D22 |
| 398 | D22 | D22 | tBu | D22 | D22 |
| 399 | D23 | D23 | tBu | D23 | tBu |
| 400 | D23 | D23 | tBu | tBu | D23 |
| 401 | D23 | D23 | tBu | D23 | D23 |
| 402 | D24 | D24 | tBu | D24 | tBu |
| 403 | D24 | D24 | tBu | tBu | D24 |
| 404 | D24 | D24 | tBu | D24 | D24 |
| 405 | D25 | D25 | tBu | D25 | tBu |
| 406 | D25 | D25 | tBu | tBu | D25 |
| 407 | D25 | D25 | tBu | D25 | D25 |
| 408 | D26 | D26 | tBu | D26 | tBu |
| 409 | D26 | D26 | tBu | tBu | D26 |
| 410 | D26 | D26 | tBu | D26 | D26 |
| 411 | D27 | D27 | tBu | D27 | tBu |
| 412 | D27 | D27 | tBu | tBu | D27 |
| 413 | D27 | D27 | tBu | D27 | D27 |
| 414 | D28 | D28 | tBu | D28 | tBu |
| 415 | D28 | D28 | tBu | tBu | D28 |
| 416 | D28 | D28 | tBu | D28 | D28 |
| 417 | D29 | D29 | tBu | D29 | tBu |
| 418 | D29 | D29 | tBu | tBu | D29 |
| 419 | D29 | D29 | tBu | D29 | D29 |
| 420 | D30 | D30 | tBu | D30 | tBu |
| 421 | D30 | D30 | tBu | tBu | D30 |
| 422 | D30 | D30 | tBu | D30 | D30 |
| 423 | D31 | D31 | tBu | D31 | tBu |
| 424 | D31 | D31 | tBu | tBu | D31 |
| 425 | D31 | D31 | tBu | D31 | D31 |
| 426 | D32 | D32 | tBu | D32 | tBu |
| 427 | D32 | D32 | tBu | tBu | D32 |
| 428 | D32 | D32 | tBu | D32 | D32 |
| 429 | D33 | D33 | tBu | D33 | tBu |
| 430 | D33 | D33 | tBu | tBu | D33 |
| 431 | D33 | D33 | tBu | D33 | D33 |
| 432 | D34 | D34 | tBu | D34 | tBu |
| 433 | D34 | D34 | tBu | tBu | D34 |
| 434 | D34 | D34 | tBu | D34 | D34 |
| 435 | D35 | D35 | tBu | D35 | tBu |
| 436 | D35 | D35 | tBu | tBu | D35 |
| 437 | D35 | D35 | tBu | D35 | D35 |
| 438 | D36 | D36 | tBu | D36 | tBu |
| 439 | D36 | D36 | tBu | tBu | D36 |
| 440 | D36 | D36 | tBu | D36 | D36 |
| 441 | D37 | D37 | tBu | D37 | tBu |
| 442 | D37 | D37 | tBu | tBu | D37 |
| 443 | D37 | D37 | tBu | D37 | D37 |
| 444 | D1 | D1 | methoxy | D1 | methoxy |
| 445 | D1 | D1 | methoxy | methoxy | D1 |
| 446 | D1 | D1 | methoxy | D1 | D1 |
| 447 | D2 | D2 | methoxy | D2 | methoxy |
| 448 | D2 | D2 | methoxy | methoxy | D2 |
| 449 | D2 | D2 | methoxy | D2 | D2 |
| 450 | D3 | D3 | methoxy | D3 | methoxy |
| 451 | D3 | D3 | methoxy | methoxy | D3 |
| 452 | D3 | D3 | methoxy | D3 | D3 |
| 453 | D4 | D4 | methoxy | D4 | methoxy |
| 454 | D4 | D4 | methoxy | methoxy | D4 |
| 455 | D4 | D4 | methoxy | D4 | D4 |
| 456 | D5 | D5 | methoxy | D5 | methoxy |
| 457 | D5 | D5 | methoxy | methoxy | D5 |
| 458 | D5 | D5 | methoxy | D5 | D5 |
| 459 | D6 | D6 | methoxy | D6 | methoxy |
| 460 | D6 | D6 | methoxy | methoxy | D6 |
| 461 | D6 | D6 | methoxy | D6 | D6 |
| 462 | D7 | D7 | methoxy | D7 | methoxy |
| 463 | D7 | D7 | methoxy | methoxy | D7 |
| 464 | D7 | D7 | methoxy | D7 | D7 |
| 465 | D8 | D8 | methoxy | D8 | methoxy |
| 466 | D8 | D8 | methoxy | methoxy | D8 |
| 467 | D8 | D8 | methoxy | D8 | D8 |
| 468 | D9 | D9 | methoxy | D9 | methoxy |
| 469 | D9 | D9 | methoxy | methoxy | D9 |
| 470 | D9 | D9 | methoxy | D9 | D9 |
| 471 | D10 | D10 | methoxy | D10 | methoxy |
| 472 | D10 | D10 | methoxy | methoxy | D10 |
| 473 | D10 | D10 | methoxy | D10 | D10 |
| 474 | D11 | D11 | methoxy | D11 | methoxy |
| 475 | D11 | D11 | methoxy | methoxy | D11 |
| 476 | D11 | D11 | methoxy | D11 | D11 |
| 477 | D12 | D12 | methoxy | D12 | methoxy |
| 478 | D12 | D12 | methoxy | methoxy | D12 |
| 479 | D12 | D12 | methoxy | D12 | D12 |
| 480 | D13 | D13 | methoxy | D13 | methoxy |
| 481 | D13 | D13 | methoxy | methoxy | D13 |
| 482 | D13 | D13 | methoxy | D13 | D13 |
| 483 | D14 | D14 | methoxy | D14 | methoxy |
| 484 | D14 | D14 | methoxy | methoxy | D14 |
| 485 | D14 | D14 | methoxy | D14 | D14 |
| 486 | D15 | D15 | methoxy | D15 | methoxy |
| 487 | D15 | D15 | methoxy | methoxy | D15 |
| 488 | D15 | D15 | methoxy | D15 | D15 |
| 489 | D16 | D16 | methoxy | D16 | methoxy |
| 490 | D16 | D16 | methoxy | methoxy | D16 |
| 491 | D16 | D16 | methoxy | D16 | D16 |
| 492 | D17 | D17 | methoxy | D17 | methoxy |
| 493 | D17 | D17 | methoxy | methoxy | D17 |
| 494 | D17 | D17 | methoxy | D17 | D17 |
| 495 | D18 | D18 | methoxy | D18 | methoxy |
| 496 | D18 | D18 | methoxy | methoxy | D18 |
| 497 | D18 | D18 | methoxy | D18 | D18 |
| 498 | D19 | D19 | methoxy | D19 | methoxy |
| 499 | D19 | D19 | methoxy | methoxy | D19 |
| 500 | D19 | D19 | methoxy | D19 | D19 |
| 501 | D20 | D20 | methoxy | D20 | methoxy |
| 502 | D20 | D20 | methoxy | methoxy | D20 |
| 503 | D20 | D20 | methoxy | D20 | D20 |
| 504 | D21 | D21 | methoxy | D21 | methoxy |
| 505 | D21 | D21 | methoxy | methoxy | D21 |
| 506 | D21 | D21 | methoxy | D21 | D21 |
| 507 | D22 | D22 | methoxy | D22 | methoxy |
| 508 | D22 | D22 | methoxy | methoxy | D22 |
| 509 | D22 | D22 | methoxy | D22 | D22 |
| 510 | D23 | D23 | methoxy | D23 | methoxy |
| 511 | D23 | D23 | methoxy | methoxy | D23 |
| 512 | D23 | D23 | methoxy | D23 | D23 |
| 513 | D24 | D24 | methoxy | D24 | methoxy |
| 514 | D24 | D24 | methoxy | methoxy | D24 |
| 515 | D24 | D24 | methoxy | D24 | D24 |
| 516 | D25 | D25 | methoxy | D25 | methoxy |
| 517 | D25 | D25 | methoxy | methoxy | D25 |
| 518 | D25 | D25 | methoxy | D25 | D25 |
| 519 | D26 | D26 | methoxy | D26 | methoxy |
| 520 | D26 | D26 | methoxy | methoxy | D26 |
| 521 | D26 | D26 | methoxy | D26 | D26 |
| 522 | D27 | D27 | methoxy | D27 | methoxy |
| 523 | D27 | D27 | methoxy | methoxy | D27 |
| 524 | D27 | D27 | methoxy | D27 | D27 |
| 525 | D28 | D28 | methoxy | D28 | methoxy |
| 526 | D28 | D28 | methoxy | methoxy | D28 |
| 527 | D28 | D28 | methoxy | D28 | D28 |
| 528 | D29 | D29 | methoxy | D29 | methoxy |
| 529 | D29 | D29 | methoxy | methoxy | D29 |
| 530 | D29 | D29 | methoxy | D29 | D29 |
| 531 | D30 | D30 | methoxy | D30 | methoxy |
| 532 | D30 | D30 | methoxy | methoxy | D30 |
| 533 | D30 | D30 | methoxy | D30 | D30 |
| 534 | D31 | D31 | methoxy | D31 | methoxy |
| 535 | D31 | D31 | methoxy | methoxy | D31 |
| 536 | D31 | D31 | methoxy | D31 | D31 |
| 537 | D32 | D32 | methoxy | D32 | methoxy |
| 538 | D32 | D32 | methoxy | methoxy | D32 |
| 539 | D32 | D32 | methoxy | D32 | D32 |
| 540 | D33 | D33 | methoxy | D33 | methoxy |
| 541 | D33 | D33 | methoxy | methoxy | D33 |
| 542 | D33 | D33 | methoxy | D33 | D33 |
| 543 | D34 | D34 | methoxy | D34 | methoxy |
| 544 | D34 | D34 | methoxy | methoxy | D34 |
| 545 | D34 | D34 | methoxy | D34 | D34 |
| 546 | D35 | D35 | methoxy | D35 | methoxy |
| 547 | D35 | D35 | methoxy | methoxy | D35 |
| 548 | D35 | D35 | methoxy | D35 | D35 |
| 549 | D36 | D36 | methoxy | D36 | methoxy |
| 550 | D36 | D36 | methoxy | methoxy | D36 |
| 551 | D36 | D36 | methoxy | D36 | D36 |
| 552 | D37 | D37 | methoxy | D37 | methoxy |
| 553 | D37 | D37 | methoxy | methoxy | D37 |
| 554 | D37 | D37 | methoxy | D37 | D37 |
| 555 | D1 | D1 | methyl | H | D1 |
| 556 | D2 | D2 | methyl | H | D2 |
| 557 | D3 | D3 | methyl | H | D3 |
| 558 | D4 | D4 | methyl | H | D4 |
| 559 | D5 | D5 | methyl | H | D5 |
| 560 | D6 | D6 | methyl | H | D6 |
| 561 | D7 | D7 | methyl | H | D7 |
| 562 | D8 | D8 | methyl | H | D8 |
| 563 | D9 | D9 | methyl | H | D9 |
| 564 | D10 | D10 | methyl | H | D10 |
| 565 | D11 | D11 | methyl | H | D11 |
| 566 | D12 | D12 | methyl | H | D12 |
| 567 | D13 | D13 | methyl | H | D13 |
| 568 | D14 | D14 | methyl | H | D14 |
| 569 | D15 | D15 | methyl | H | D15 |
| 570 | D16 | D16 | methyl | H | D16 |
| 571 | D17 | D17 | methyl | H | D17 |
| 572 | D18 | D18 | methyl | H | D18 |
| 573 | D19 | D19 | methyl | H | D19 |
| 574 | D20 | D20 | methyl | H | D20 |
| 575 | D21 | D21 | methyl | H | D21 |
| 576 | D22 | D22 | methyl | H | D22 |
| 577 | D23 | D23 | methyl | H | D23 |
| 578 | D24 | D24 | methyl | H | D24 |
| 579 | D25 | D25 | methyl | H | D25 |
| 580 | D26 | D26 | methyl | H | D26 |
| 581 | D27 | D27 | methyl | H | D27 |
| 582 | D28 | D28 | methyl | H | D28 |
| 583 | D29 | D29 | methyl | H | D29 |
| 584 | D30 | D30 | methyl | H | D30 |
| 585 | D31 | D31 | methyl | H | D31 |
| 586 | D32 | D32 | methyl | H | D32 |
| 587 | D33 | D33 | methyl | H | D33 |
| 588 | D34 | D34 | methyl | H | D34 |
| 589 | D35 | D35 | methyl | H | D35 |
| 590 | D36 | D36 | methyl | H | D36 |
| 591 | D37 | D37 | methyl | H | D37 |
| 592 | D1 | D1 | H | methyl | D1 |
| 593 | D2 | D2 | H | methyl | D2 |
| 594 | D3 | D3 | H | methyl | D3 |
| 595 | D4 | D4 | H | methyl | D4 |
| 596 | D5 | D5 | H | methyl | D5 |
| 597 | D6 | D6 | H | methyl | D6 |
| 598 | D7 | D7 | H | methyl | D7 |
| 599 | D8 | D8 | H | methyl | D8 |
| 600 | D9 | D9 | H | methyl | D9 |
| 601 | D10 | D10 | H | methyl | D10 |
| 602 | D11 | D11 | H | methyl | D11 |
| 603 | D12 | D12 | H | methyl | D12 |
| 604 | D13 | D13 | H | methyl | D13 |
| 605 | D14 | D14 | H | methyl | D14 |
| 606 | D15 | D15 | H | methyl | D15 |
| 607 | D16 | D16 | H | methyl | D16 |
| 608 | D17 | D17 | H | methyl | D17 |
| 609 | D18 | D18 | H | methyl | D18 |
| 610 | D19 | D19 | H | methyl | D19 |
| 611 | D20 | D20 | H | methyl | D20 |
| 612 | D21 | D21 | H | methyl | D21 |
| 613 | D22 | D22 | H | methyl | D22 |
| 614 | D23 | D23 | H | methyl | D23 |
| 615 | D24 | D24 | H | methyl | D24 |
| 616 | D25 | D25 | H | methyl | D25 |
| 617 | D26 | D26 | H | methyl | D26 |
| 618 | D27 | D27 | H | methyl | D27 |
| 619 | D28 | D28 | H | methyl | D28 |
| 620 | D29 | D29 | H | methyl | D29 |
| 621 | D30 | D30 | H | methyl | D30 |
| 622 | D31 | D31 | H | methyl | D31 |
| 623 | D32 | D32 | H | methyl | D32 |
| 624 | D33 | D33 | H | methyl | D33 |
| 625 | D34 | D34 | H | methyl | D34 |
| 626 | D35 | D35 | H | methyl | D35 |
| 627 | D36 | D36 | H | methyl | D36 |
| 628 | D37 | D37 | H | methyl | D37 |
| 629 | D1 | D1 | H | D1 | methyl |
| 630 | D2 | D2 | H | D2 | methyl |
| 631 | D3 | D3 | H | D3 | methyl |
| 632 | D4 | D4 | H | D4 | methyl |
| 633 | D5 | D5 | H | D5 | methyl |
| 634 | D6 | D6 | H | D6 | methyl |
| 635 | D7 | D7 | H | D7 | methyl |
| 636 | D8 | D8 | H | D8 | methyl |
| 637 | D9 | D9 | H | D9 | methyl |
| 638 | D10 | D10 | H | D10 | methyl |
| 639 | D11 | D11 | H | D11 | methyl |
| 640 | D12 | D12 | H | D12 | methyl |
| 641 | D13 | D13 | H | D13 | methyl |
| 642 | D14 | D14 | H | D14 | methyl |
| 643 | D15 | D15 | H | D15 | methyl |
| 644 | D16 | D16 | H | D16 | methyl |
| 645 | D17 | D17 | H | D17 | methyl |
| 646 | D18 | D18 | H | D18 | methyl |
| 647 | D19 | D19 | H | D19 | methyl |
| 648 | D20 | D20 | H | D20 | methyl |
| 649 | D21 | D21 | H | D21 | methyl |
| 650 | D22 | D22 | H | D22 | methyl |
| 651 | D23 | D23 | H | D23 | methyl |
| 652 | D24 | D24 | H | D24 | methyl |
| 653 | D25 | D25 | H | D25 | methyl |
| 654 | D26 | D26 | H | D26 | methyl |
| 655 | D27 | D27 | H | D27 | methyl |
| 656 | D28 | D28 | H | D28 | methyl |
| 657 | D29 | D29 | H | D29 | methyl |
| 658 | D30 | D30 | H | D30 | methyl |
| 659 | D31 | D31 | H | D31 | methyl |
| 660 | D32 | D32 | H | D32 | methyl |
| 661 | D33 | D33 | H | D33 | methyl |
| 662 | D34 | D34 | H | D34 | methyl |
| 663 | D35 | D35 | H | D35 | methyl |
| 664 | D36 | D36 | H | D36 | methyl |
| 665 | D37 | D37 | H | D37 | methyl |
| 666 | D1 | D1 | methoxy | H | D1 |
| 667 | D2 | D2 | methoxy | H | D2 |
| 668 | D3 | D3 | methoxy | H | D3 |
| 669 | D4 | D4 | methoxy | H | D4 |
| 670 | D5 | D5 | methoxy | H | D5 |
| 671 | D6 | D6 | methoxy | H | D6 |
| 672 | D7 | D7 | methoxy | H | D7 |
| 673 | D8 | D8 | methoxy | H | D8 |
| 674 | D9 | D9 | methoxy | H | D9 |
| 675 | D10 | D10 | methoxy | H | D10 |
| 676 | D11 | D11 | methoxy | H | D11 |
| 677 | D12 | D12 | methoxy | H | D12 |
| 678 | D13 | D13 | methoxy | H | D13 |
| 679 | D14 | D14 | methoxy | H | D14 |
| 680 | D15 | D15 | methoxy | H | D15 |
| 681 | D16 | D16 | methoxy | H | D16 |
| 682 | D17 | D17 | methoxy | H | D17 |
| 683 | D18 | D18 | methoxy | H | D18 |
| 684 | D19 | D19 | methoxy | H | D19 |
| 685 | D20 | D20 | methoxy | H | D20 |
| 686 | D21 | D21 | methoxy | H | D21 |
| 687 | D22 | D22 | methoxy | H | D22 |
| 688 | D23 | D23 | methoxy | H | D23 |
| 689 | D24 | D24 | methoxy | H | D24 |
| 690 | D25 | D25 | methoxy | H | D25 |
| 691 | D26 | D26 | methoxy | H | D26 |
| 692 | D27 | D27 | methoxy | H | D27 |
| 693 | D28 | D28 | methoxy | H | D28 |
| 694 | D29 | D29 | methoxy | H | D29 |
| 695 | D30 | D30 | methoxy | H | D30 |
| 696 | D31 | D31 | methoxy | H | D31 |
| 697 | D32 | D32 | methoxy | H | D32 |
| 698 | D33 | D33 | methoxy | H | D33 |
| 699 | D34 | D34 | methoxy | H | D34 |
| 700 | D35 | D35 | methoxy | H | D35 |
| 701 | D36 | D36 | methoxy | H | D36 |
| 702 | D37 | D37 | methoxy | H | D37 |
| 703 | D1 | D1 | H | methoxy | D1 |
| 704 | D2 | D2 | H | methoxy | D2 |
| 705 | D3 | D3 | H | methoxy | D3 |
| 706 | D4 | D4 | H | methoxy | D4 |
| 707 | D5 | D5 | H | methoxy | D5 |
| 708 | D6 | D6 | H | methoxy | D6 |
| 709 | D7 | D7 | H | methoxy | D7 |
| 710 | D8 | D8 | H | methoxy | D8 |
| 711 | D9 | D9 | H | methoxy | D9 |
| 712 | D10 | D10 | H | methoxy | D10 |
| 713 | D11 | D11 | H | methoxy | D11 |
| 714 | D12 | D12 | H | methoxy | D12 |
| 715 | D13 | D13 | H | methoxy | D13 |
| 716 | D14 | D14 | H | methoxy | D14 |
| 717 | D15 | D15 | H | methoxy | D15 |
| 718 | D16 | D16 | H | methoxy | D16 |
| 719 | D17 | D17 | H | methoxy | D17 |
| 720 | D18 | D18 | H | methoxy | D18 |
| 721 | D19 | D19 | H | methoxy | D19 |
| 722 | D20 | D20 | H | methoxy | D20 |
| 723 | D21 | D21 | H | methoxy | D21 |
| 724 | D22 | D22 | H | methoxy | D22 |
| 725 | D23 | D23 | H | methoxy | D23 |
| 726 | D24 | D24 | H | methoxy | D24 |
| 727 | D25 | D25 | H | methoxy | D25 |
| 728 | D26 | D26 | H | methoxy | D26 |
| 729 | D27 | D27 | H | methoxy | D27 |
| 730 | D28 | D28 | H | methoxy | D28 |
| 731 | D29 | D29 | H | methoxy | D29 |
| 732 | D30 | D30 | H | methoxy | D30 |
| 733 | D31 | D31 | H | methoxy | D31 |
| 734 | D32 | D32 | H | methoxy | D32 |
| 735 | D33 | D33 | H | methoxy | D33 |
| 736 | D34 | D34 | H | methoxy | D34 |
| 737 | D35 | D35 | H | methoxy | D35 |
| 738 | D36 | D36 | H | methoxy | D36 |
| 739 | D37 | D37 | H | methoxy | D37 |
| 740 | D1 | D1 | H | D1 | methoxy |
| 741 | D2 | D2 | H | D2 | methoxy |
| 742 | D3 | D3 | H | D3 | methoxy |
| 743 | D4 | D4 | H | D4 | methoxy |
| 744 | D5 | D5 | H | D5 | methoxy |
| 745 | D6 | D6 | H | D6 | methoxy |
| 746 | D7 | D7 | H | D7 | methoxy |
| 747 | D8 | D8 | H | D8 | methoxy |
| 748 | D9 | D9 | H | D9 | methoxy |
| 749 | D10 | D10 | H | D10 | methoxy |
| 750 | D11 | D11 | H | D11 | methoxy |
| 751 | D12 | D12 | H | D12 | methoxy |
| 752 | D13 | D13 | H | D13 | methoxy |
| 753 | D14 | D14 | H | D14 | methoxy |
| 754 | D15 | D15 | H | D15 | methoxy |
| 755 | D16 | D16 | H | D16 | methoxy |
| 756 | D17 | D17 | H | D17 | methoxy |
| 757 | D18 | D18 | H | D18 | methoxy |
| 758 | D19 | D19 | H | D19 | methoxy |
| 759 | D20 | D20 | H | D20 | methoxy |
| 760 | D21 | D21 | H | D21 | methoxy |
| 761 | D22 | D22 | H | D22 | methoxy |
| 762 | D23 | D23 | H | D23 | methoxy |
| 763 | D24 | D24 | H | D24 | methoxy |
| 764 | D25 | D25 | H | D25 | methoxy |
| 765 | D26 | D26 | H | D26 | methoxy |
| 766 | D27 | D27 | H | D27 | methoxy |
| 767 | D28 | D28 | H | D28 | methoxy |
| 768 | D29 | D29 | H | D29 | methoxy |
| 769 | D30 | D30 | H | D30 | methoxy |
| 770 | D31 | D31 | H | D31 | methoxy |
| 771 | D32 | D32 | H | D32 | methoxy |
| 772 | D33 | D33 | H | D33 | methoxy |
| 773 | D34 | D34 | H | D34 | methoxy |
| 774 | D35 | D35 | H | D35 | methoxy |
| 775 | D36 | D36 | H | D36 | methoxy |
| 776 | D37 | D37 | H | D37 | methoxy |
| 777 | D1 | D1 | morpholine | D1 | D1 |
| 778 | D2 | D2 | morpholine | D2 | D2 |
| 779 | D3 | D3 | morpholine | D3 | D3 |
| 780 | D4 | D4 | morpholine | D4 | D4 |
| 781 | D5 | D5 | morpholine | D5 | D5 |
| 782 | D6 | D6 | morpholine | D6 | D6 |
| 783 | D7 | D7 | morpholine | D7 | D7 |
| 784 | D8 | D8 | morpholine | D8 | D8 |
| 785 | D9 | D9 | morpholine | D9 | D9 |
| 786 | D10 | D10 | morpholine | D10 | D10 |
| 787 | D11 | D11 | morpholine | D11 | D11 |
| 788 | D12 | D12 | morpholine | D12 | D12 |
| 789 | D13 | D13 | morpholine | D13 | D13 |
| 790 | D14 | D14 | morpholine | D14 | D14 |
| 791 | D15 | D15 | morpholine | D15 | D15 |
| 792 | D16 | D16 | morpholine | D16 | D16 |
| 793 | D17 | D17 | morpholine | D17 | D17 |
| 794 | D18 | D18 | morpholine | D18 | D18 |
| 795 | D19 | D19 | morpholine | D19 | D19 |
| 796 | D20 | D20 | morpholine | D20 | D20 |
| 797 | D21 | D21 | morpholine | D21 | D21 |
| 798 | D22 | D22 | morpholine | D22 | D22 |
| 799 | D23 | D23 | morpholine | D23 | D23 |
| 800 | D24 | D24 | morpholine | D24 | D24 |
| 801 | D25 | D25 | morpholine | D25 | D25 |
| 802 | D26 | D26 | morpholine | D26 | D26 |
| 803 | D27 | D27 | morpholine | D27 | D27 |
| 804 | D28 | D28 | morpholine | D28 | D28 |
| 805 | D29 | D29 | morpholine | D29 | D29 |
| 806 | D30 | D30 | morpholine | D30 | D30 |
| 807 | D31 | D31 | morpholine | D31 | D31 |
| 808 | D32 | D32 | morpholine | D32 | D32 |
| 809 | D33 | D33 | morpholine | D33 | D33 |
| 810 | D34 | D34 | morpholine | D34 | D34 |
| 811 | D35 | D35 | morpholine | D35 | D35 |
| 812 | D36 | D36 | morpholine | D36 | D36 |
| 813 | D37 | D37 | morpholine | D37 | D37 |
| 814 | D38 | D38 | H | D38 | H |
| 815 | D38 | D38 | H | H | D38 |
| 816 | D38 | D38 | H | D38 | D38 |
| 817 | D38 | D38 | methyl | D38 | methyl |
| 818 | D38 | D38 | methyl | methyl | D38 |
| 819 | D38 | D38 | methyl | D38 | D38 |
| 820 | D38 | D38 | isopropyl | D38 | isopropyl |
| 821 | D38 | D38 | isopropyl | isopropyl | D38 |
| 822 | D38 | D38 | isopropyl | D38 | D38 |
| 823 | D38 | D38 | tBu | D38 | tBu |
| 824 | D38 | D38 | tBu | tBu | D38 |
| 825 | D38 | D38 | tBu | D38 | D38 |
| 826 | D38 | D38 | methoxy | D38 | methoxy |
| 827 | D38 | D38 | methoxy | methoxy | D38 |
| 828 | D38 | D38 | methoxy | D38 | D38 |
| 829 | D38 | D38 | methyl | H | D38 |
| 830 | D38 | D38 | H | methyl | D38 |
| 831 | D38 | D38 | H | D38 | methyl |
| 832 | D38 | D38 | methoxy | H | D38 |
| 833 | D38 | D38 | H | methoxy | D38 |
| 834 | D38 | D38 | H | D38 | methoxy |
| 835 | D38 | D38 | morpholine | D38 | D38 |
| 836 | D39 | D39 | H | D39 | H |
| 837 | D39 | D39 | H | H | D39 |
| 838 | D39 | D39 | H | D39 | D39 |
| 839 | D39 | D39 | methyl | D39 | methyl |
| 840 | D39 | D39 | methyl | methyl | D39 |
| 841 | D39 | D39 | methyl | D39 | D39 |
| 842 | D39 | D39 | isopropyl | D39 | isopropyl |
| 843 | D39 | D39 | isopropyl | isopropyl | D39 |
| 844 | D39 | D39 | isopropyl | D39 | D39 |
| 845 | D39 | D39 | tBu | D39 | tBu |
| 846 | D39 | D39 | tBu | tBu | D39 |
| 847 | D39 | D39 | tBu | D39 | D39 |
| 848 | D39 | D39 | methoxy | D39 | methoxy |
| 849 | D39 | D39 | methoxy | methoxy | D39 |
| 850 | D39 | D39 | methoxy | D39 | D39 |
| 851 | D39 | D39 | methyl | H | D39 |
| 852 | D39 | D39 | H | methyl | D39 |
| 853 | D39 | D39 | H | D39 | methyl |
| 854 | D39 | D39 | methoxy | H | D39 |
| 855 | D39 | D39 | H | methoxy | D39 |
| 856 | D39 | D39 | H | D39 | methoxy |
| 857 | D39 | D39 | morpholine | D39 | D39 |
| 858 | D40 | D40 | H | D40 | H |
| 859 | D40 | D40 | H | H | D40 |
| 860 | D40 | D40 | H | D40 | D40 |
| 861 | D40 | D40 | methyl | D40 | methyl |
| 862 | D40 | D40 | methyl | methyl | D40 |
| 863 | D40 | D40 | methyl | D40 | D40 |
| 864 | D40 | D40 | isopropyl | D40 | isopropyl |
| 865 | D40 | D40 | isopropyl | isopropyl | D40 |
| 866 | D40 | D40 | isopropyl | D40 | D40 |
| 867 | D40 | D40 | tBu | D40 | tBu |
| 868 | D40 | D40 | tBu | tBu | D40 |
| 869 | D40 | D40 | tBu | D40 | D40 |
| 870 | D40 | D40 | methoxy | D40 | methoxy |
| 871 | D40 | D40 | methoxy | methoxy | D40 |
| 872 | D40 | D40 | methoxy | D40 | D40 |
| 873 | D40 | D40 | methyl | H | D40 |
| 874 | D40 | D40 | H | methyl | D40 |
| 875 | D40 | D40 | H | D40 | methyl |
| 876 | D40 | D40 | methoxy | H | D40 |
| 877 | D40 | D40 | H | methoxy | D40 |
| 878 | D40 | D40 | H | D40 | methoxy |
| 879 | D40 | D40 | morpholine | D40 | D40 |
| 880 | D41 | D41 | H | D41 | H |
| 881 | D41 | D41 | H | H | D41 |
| 882 | D41 | D41 | H | D41 | 041 |
| 883 | D41 | D41 | methyl | D41 | methyl |
| 884 | D41 | D41 | methyl | methyl | D41 |
| 885 | D41 | D41 | methyl | D41 | D41 |
| 886 | D41 | D41 | isopropyl | D41 | isopropyl |
| 887 | D41 | D41 | isopropyl | isopropyl | D41 |
| 888 | D41 | D41 | isopropyl | D41 | D41 |
| 889 | D41 | D41 | tBu | D41 | tBu |
| 890 | D41 | D41 | tBu | tBu | D41 |
| 891 | D41 | D41 | tBu | D41 | D41 |
| 892 | D41 | D41 | methoxy | D41 | methoxy |
| 893 | D41 | D41 | methoxy | methoxy | D41 |
| 894 | D41 | D41 | methoxy | D41 | D41 |
| 895 | D41 | D41 | methyl | H | D41 |
| 896 | D41 | D41 | H | methyl | D41 |
| 897 | D41 | D41 | H | D41 | methyl |
| 898 | D41 | D41 | methoxy | H | D41 |
| 899 | D41 | D41 | H | methoxy | D41 |
| 900 | D41 | D41 | H | D41 | methoxy |
| 901 | D41 | D41 | morpholine | D41 | D41 |

Next, compounds represented by the following general formula (D) are described below.

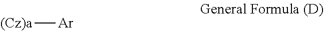

In the general formula (D):

Cz represents a 9-carbazolyl group having a substituent at at least one of 1-position and 8-position (here, at least one carbon atom at the 1- to 8-positions constituting the ring skeleton of the carbazole ring of the 9-carbazolyl group may be substituted with a nitrogen atom, but both the 1-position and the 8-position are not substituted with a nitrogen atom, and each benzene ring constituting the 9-carbazolyl group may be condensed with any other ring),
Ar represents a benzene ring having a substituent containing a structural unit having a positive Hammett constant up (but excepting a cyano group), or a biphenyl ring having a substituent containing a structural unit having a positive Hammett constant up (but excepting a cyano group),
a represents an integer of 1 or more, but is not more than the maximum number of the substituents with which the benzene ring or the biphenyl ring of Ar may be substituted. When a is 2 or more, plural Cz's may be the same as or different from each other.

The general formula (D) includes the following general formula (D1).

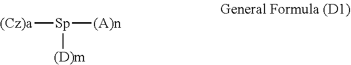

In the general formula (D1):
Sp represents a benzene ring or a biphenyl ring,
Cz represents a 9-carbazolyl group having a substituent at at least one of 1-position and 8-position (here, at least one carbon atom at the 1- to 8-positions constituting the ring skeleton of the carbazole ring of the 9-carbazolyl group may be substituted with a nitrogen atom, but both the 1-position and the 8-position are not substituted with a nitrogen atom, and each benzene ring constituting the 9-carbazolyl group may be condensed with any other ring),
D represents a substituent having a negative Hammett constant up,
A represents a substituent having a positive Hammett constant up (but excepting a cyano group),
a represents an integer of 1 or more, m represents an integer of 0 or more, n represents an integer of 1 or more, but a+m+n is not more than the maximum number of the substituents with which the benzene ring or the biphenyl ring represented by Sp may be substituted. When a is 2 or more, plural Cz's may be the same as or different from each other. When m is 2 or more, plural D's may be the same as or different from each other. When n is 2 or more, plural A's may be the same as or different from each other.

The general formula (D) also includes the following general formula (D2).

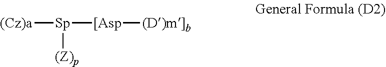

In the general formula (D2):
Sp represents a benzene ring or a biphenyl ring,
Cz represents a 9-carbazolyl group having a substituent at at least one of 1-position and 8-position (here, at least one carbon atom at the 1- to 8-positions constituting the ring skeleton of the carbazole ring of the 9-carbazolyl group may be substituted with a nitrogen atom, but both the 1-position and the 8-position are not substituted with a nitrogen atom, and each benzene ring constituting the 9-carbazolyl group may be condensed with any other ring),
Z represents a substituent except Cz and [Aₛₚ-(D′)m′],
Aₛₚ represents a substituent which may have a positive Hammett constant σₚ when all (D′)m's are substituted with a hydrogen atom,
D′ represents a substituent having a negative Hammett constant σₚ,
a represents an integer of 1 or more, b represents an integer of 1 or more, p represents an integer o 0 or more, but a+b+p is not more than the maximum number of the substituents with which the benzene ring or the biphenyl ring represented by Sp may be substituted. When a is 2 or more, plural Cz's may be the same as or different from each other. When b is 2 or more, plural Aₛₚ-(D′)m's may be the same as or different from each other. When p is 2 or more, plural Z may be the same as or different from each other. m′ represents an integer of 1 or more, but is not more than a number of the maximum number of the substituents with which Aₛₚ may be substituted, minus 1. When m′ is 2 or more, plural (D′)'s may be the same as or different from each other.

Specific examples of the “9-carbazolyl group having a substituent at at least one of 1-position and 8-position” represented by Cz include the above-mentioned m-D1 to m-D23.

Specific examples of the substituent represented by D include the above-mentioned Cz and Cz-1 to Cz-12.

Specific examples (A-1 to A-78) of the substituent represented by A are shown below. * indicates a bonding position.

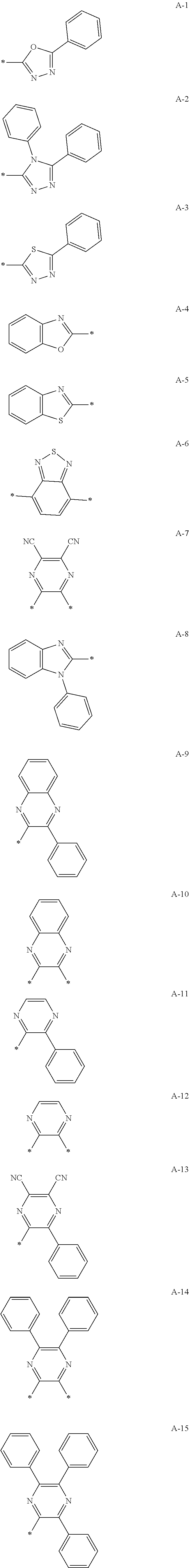

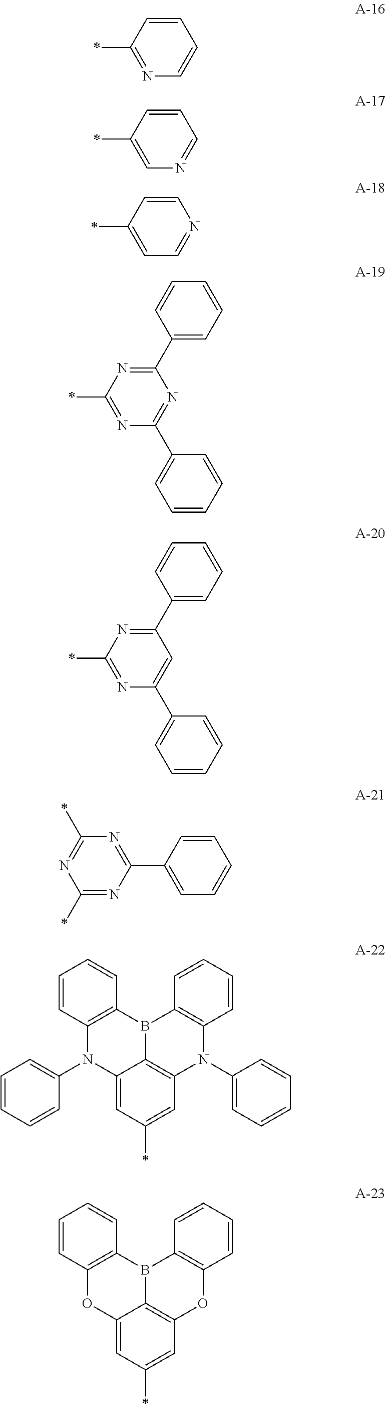

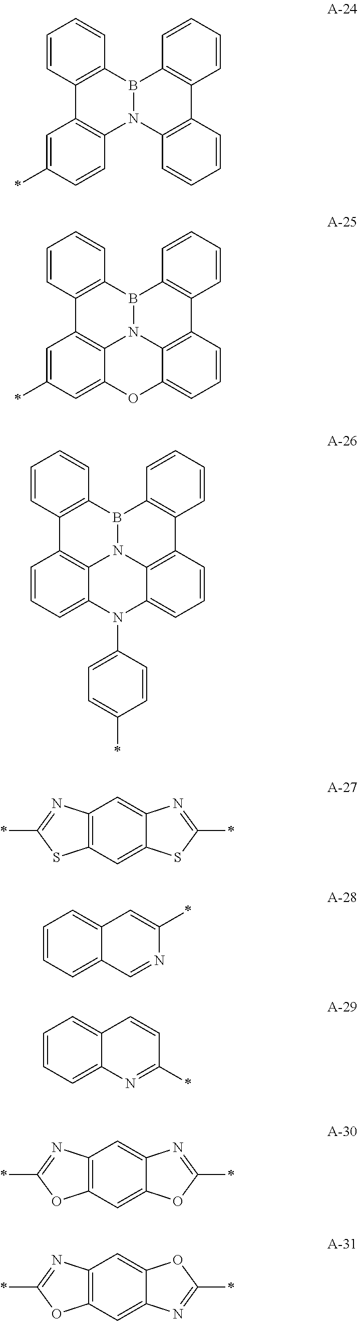

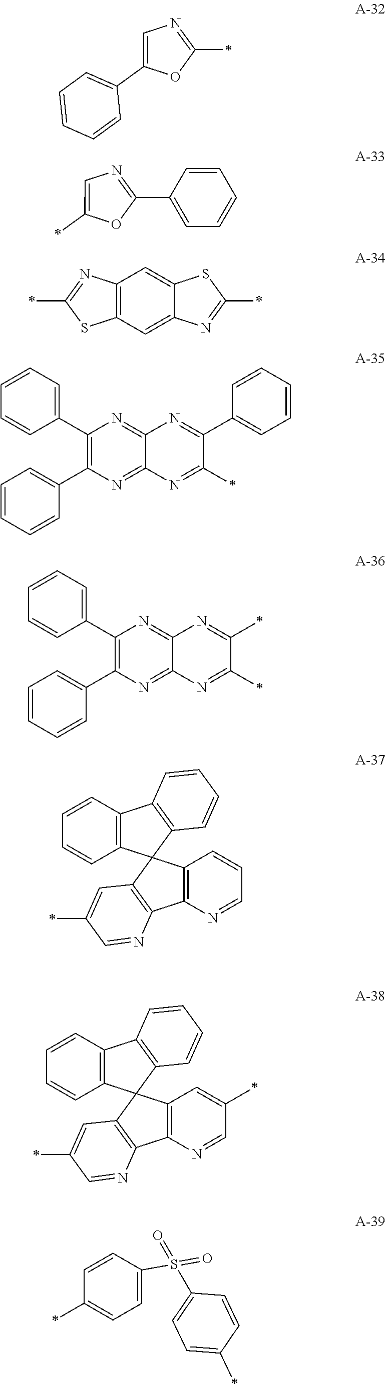

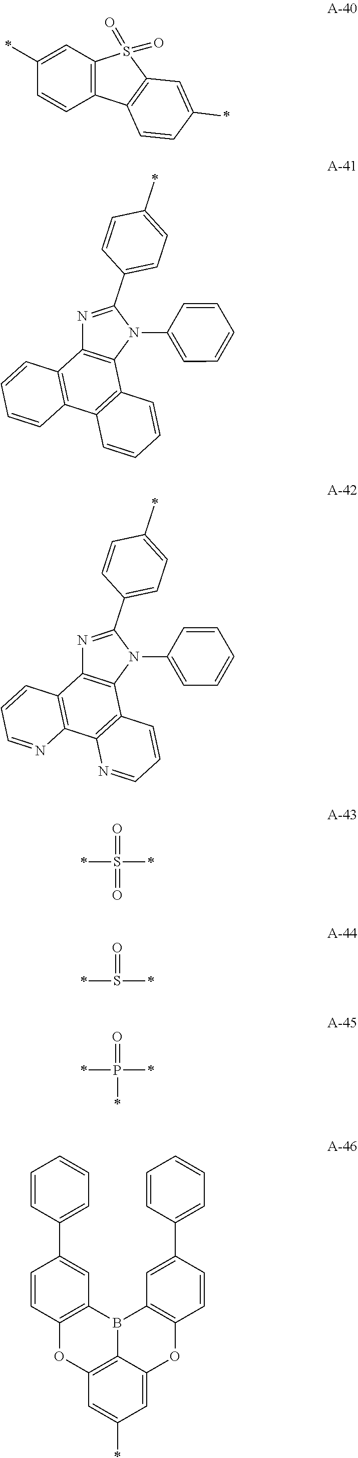

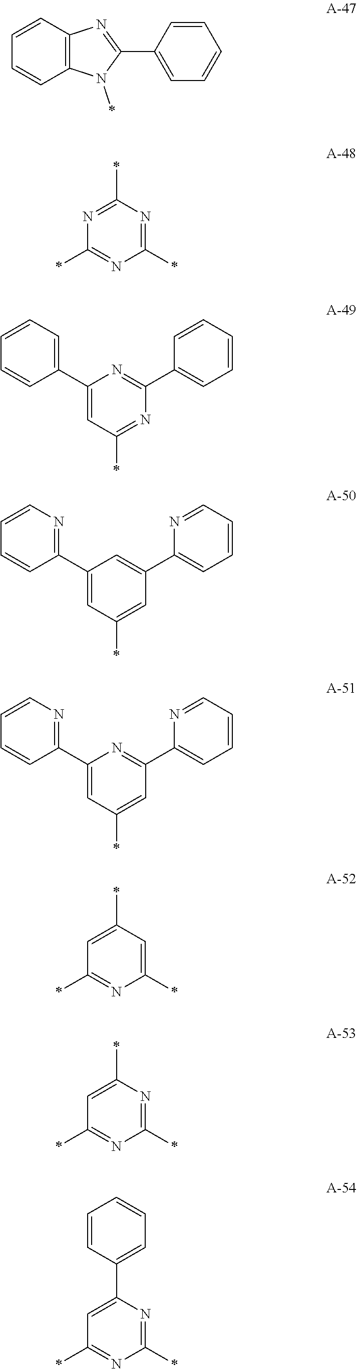

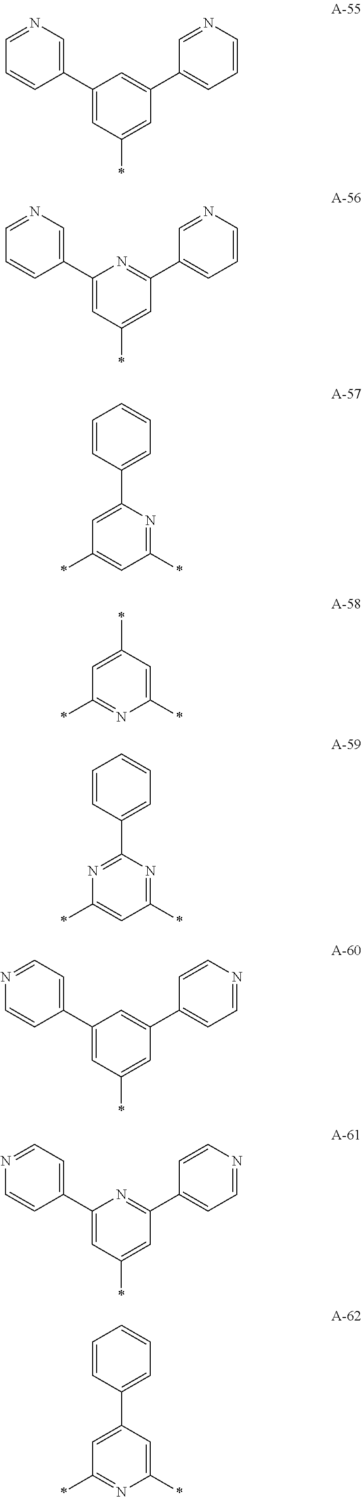

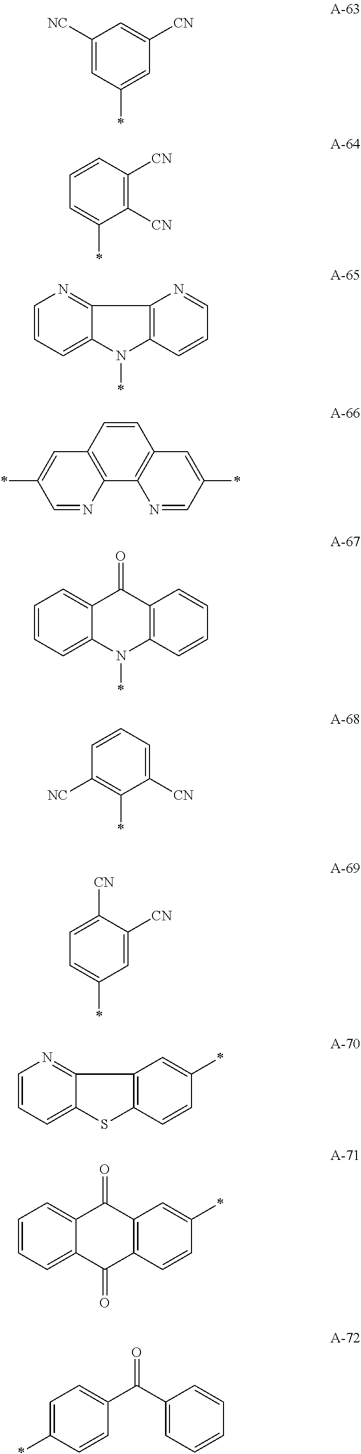

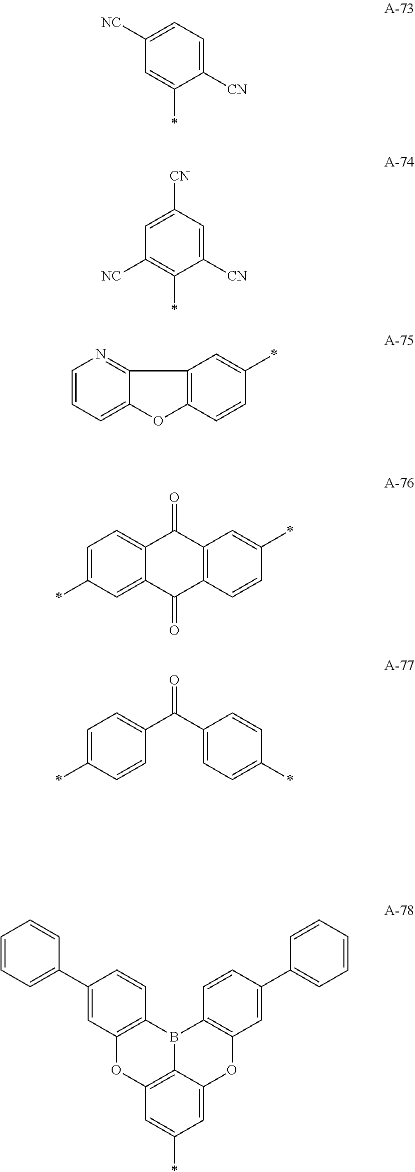 [SMILES: *c1cc(C#N)ccc1C#N ; *c1c(C#N)cc(C#N)cc1C#N ; *c1ccc2oc3cccnc3c2c1 ; *c1ccc2c(c1)C(=O)c1ccc(*)cc1C2=O ; *c1ccc(C(=O)c2ccc(*)cc2)cc1 ; *c1cc2c3c(c1)Oc1cc(-c4ccccc4)ccc1B3c1ccc(-c3ccccc3)cc1O2]

The compounds represented by the general formula (D) are preferably compounds represented by the following general formulae S-1 to S-18. R¹¹ to R¹⁵, R²¹ to R²⁴, and R²⁶ to R²⁹ each independently represent any of the substituent Cz, the substituent D or the substituent A. However, the general formulae S-1 to S-18 each have at least one substituent Cz and at least one substituent A in any of R¹¹ to R¹⁵, R²¹ to R²⁴, and R²⁶ to R²⁹ therein. R^{a}, R^{b}, R^{c}, and R^{d} each independently represent an alkyl group. R^{a}'s, R^{b}'s, R^{c}'s, and R^{d}'s each may be the same as or different from each other.

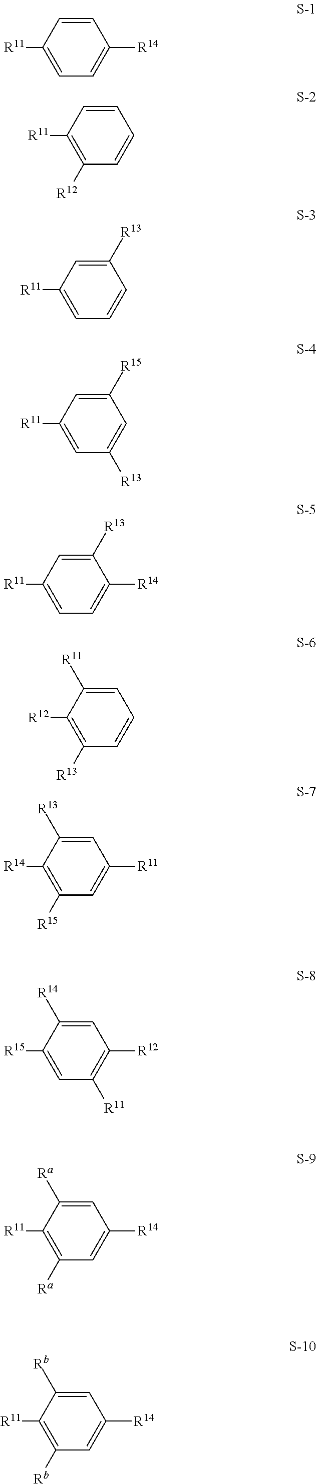

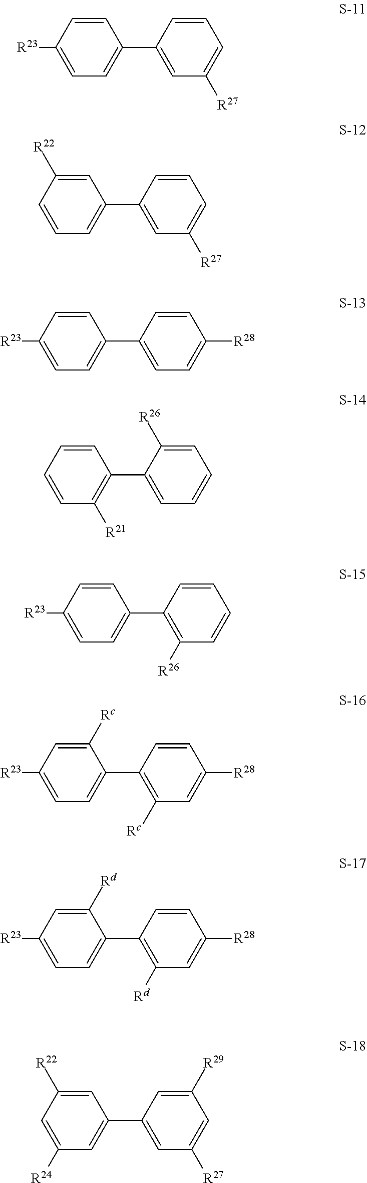

Specific examples of the compounds represented by the general formula (D) include compounds represented by the following general formula (D3) in which X¹ to X¹⁰ each represent a group shown in the following Tables 11 to 13, and t represents a number shown in the following Tables 11 to 13.

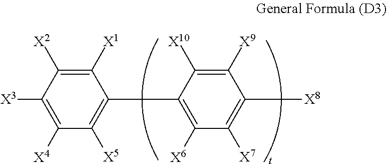

TABLE 11

| No. | t | x1 | x2 | x3 | x4 | x5 | x8 |
|---|---|---|---|---|---|---|---|
| 1 | 0 | m-D1 | H | H | A-1 | H | H |
| 2 | 0 | m-D1 | H | H | A-2 | H | H |
| 3 | 0 | m-D1 | H | H | A-3 | H | H |
| 4 | 0 | m-D1 | H | H | A-4 | H | H |
| 5 | 0 | m-D1 | H | H | A-5 | H | H |
| 6 | 0 | m-D1 | H | H | A-6 | H | H |
| 7 | 0 | m-D1 | H | H | A-7 | H | H |
| 8 | 0 | m-D1 | H | H | A-8 | H | H |
| 9 | 0 | m-D1 | H | H | A-9 | H | H |
| 10 | 0 | m-D1 | H | H | A-10 | H | H |
| 11 | 0 | m-D1 | H | H | A-11 | H | H |
| 12 | 0 | m-D1 | H | H | A-12 | H | H |
| 13 | 0 | m-D1 | H | H | A-13 | H | H |
| 14 | 0 | m-D1 | H | H | A-14 | H | H |
| 15 | 0 | m-D1 | H | H | A-15 | H | H |
| 16 | 0 | m-D1 | H | H | A-16 | H | H |
| 17 | 0 | m-D1 | H | H | A-17 | H | H |
| 18 | 0 | m-D1 | H | H | A-18 | H | H |
| 19 | 0 | m-D1 | H | H | A-19 | H | H |
| 20 | 0 | m-D1 | H | H | A-20 | H | H |
| 21 | 0 | m-D1 | H | H | A-21 | H | H |
| 22 | 0 | m-D1 | H | H | A-22 | H | H |
| 23 | 0 | m-D1 | H | H | A-23 | H | H |
| 24 | 0 | m-D1 | H | H | A-24 | H | H |
| 25 | 0 | m-D1 | H | H | A-25 | H | H |
| 26 | 0 | m-D1 | H | H | A-26 | H | H |
| 27 | 0 | m-D1 | H | H | A-27 | H | H |
| 28 | 0 | m-D1 | H | H | A-28 | H | H |
| 29 | 0 | m-D1 | H | H | A-29 | H | H |
| 30 | 0 | m-D1 | H | H | A-30 | H | H |
| 31 | 0 | m-D1 | H | H | A-31 | H | H |
| 32 | 0 | m-D1 | H | H | A-32 | H | H |
| 33 | 0 | m-D1 | H | H | A-33 | H | H |
| 34 | 0 | m-D1 | H | H | A-34 | H | H |
| 35 | 0 | m-D1 | H | H | A-35 | H | H |
| 36 | 0 | m-D1 | H | H | A-36 | H | H |
| 37 | 0 | m-D1 | H | H | A-37 | H | H |
| 38 | 0 | m-D1 | H | H | A-38 | H | H |
| 39 | 0 | m-D1 | H | H | A-39 | H | H |
| 40 | 0 | m-D1 | H | H | A-40 | H | H |
| 41 | 0 | m-D1 | H | H | A-41 | H | H |
| 42 | 0 | m-D1 | H | H | A-42 | H | H |
| 43 | 0 | m-D1 | H | H | A-43 | H | H |
| 44 | 0 | m-D1 | H | H | A-44 | H | H |
| 45 | 0 | m-D1 | H | H | A-45 | H | H |
| 46 | 0 | m-D1 | H | H | A-46 | H | H |
| 47 | 0 | m-D1 | H | H | A-47 | H | H |
| 48 | 0 | m-D2 | H | H | A-1 | H | H |
| 49 | 0 | m-D2 | H | H | A-2 | H | H |
| 50 | 0 | m-D2 | H | H | A-3 | H | H |
| 51 | 0 | m-D2 | H | H | A-4 | H | H |
| 52 | 0 | m-D2 | H | H | A-5 | H | H |
| 53 | 0 | m-D2 | H | H | A-6 | H | H |
| 54 | 0 | m-D2 | H | H | A-7 | H | H |
| 55 | 0 | m-D2 | H | H | A-8 | H | H |
| 56 | 0 | m-D2 | H | H | A-9 | H | H |
| 57 | 0 | m-D2 | H | H | A-10 | H | H |
| 58 | 0 | m-D2 | H | H | A-11 | H | H |
| 59 | 0 | m-D2 | H | H | A-12 | H | H |
| 60 | 0 | m-D2 | H | H | A-13 | H | H |
| 61 | 0 | m-D2 | H | H | A-14 | H | H |
| 62 | 0 | m-D2 | H | H | A-15 | H | H |
| 63 | 0 | m-D2 | H | H | A-16 | H | H |
| 64 | 0 | m-D2 | H | H | A-17 | H | H |
| 65 | 0 | m-D2 | H | H | A-18 | H | H |
| 66 | 0 | m-D2 | H | H | A-19 | H | H |
| 67 | 0 | m-D2 | H | H | A-20 | H | H |
| 68 | 0 | m-D2 | H | H | A-21 | H | H |
| 69 | 0 | m-D2 | H | H | A-22 | H | H |
| 70 | 0 | m-D2 | H | H | A-23 | H | H |
| 71 | 0 | m-D2 | H | H | A-24 | H | H |
| 72 | 0 | m-D2 | H | H | A-25 | H | H |
| 73 | 0 | m-D2 | H | H | A-26 | H | H |
| 74 | 0 | m-D2 | H | H | A-27 | H | H |
| 75 | 0 | m-D2 | H | H | A-28 | H | H |
| 76 | 0 | m-D2 | H | H | A-29 | H | H |
| 77 | 0 | m-D2 | H | H | A-30 | H | H |
| 78 | 0 | m-D2 | H | H | A-31 | H | H |
| 79 | 0 | m-D2 | H | H | A-32 | H | H |
| 80 | 0 | m-D2 | H | H | A-33 | H | H |
| 81 | 0 | m-D2 | H | H | A-34 | H | H |
| 82 | 0 | m-D2 | H | H | A-35 | H | H |
| 83 | 0 | m-D2 | H | H | A-36 | H | H |
| 84 | 0 | m-D2 | H | H | A-37 | H | H |
| 85 | 0 | m-D2 | H | H | A-38 | H | H |
| 86 | 0 | m-D2 | H | H | A-39 | H | H |
| 87 | 0 | m-D2 | H | H | A-40 | H | H |
| 88 | 0 | m-D2 | H | H | A-41 | H | H |
| 89 | 0 | m-D2 | H | H | A-42 | H | H |
| 90 | 0 | m-D2 | H | H | A-43 | H | H |
| 91 | 0 | m-D2 | H | H | A-44 | H | H |
| 92 | 0 | m-D2 | H | H | A-45 | H | H |
| 93 | 0 | m-D2 | H | H | A-46 | H | H |
| 94 | 0 | m-D2 | H | H | A-47 | H | H |
| 95 | 0 | m-D3 | H | H | A-1 | H | H |
| 96 | 0 | m-D3 | H | H | A-2 | H | H |
| 97 | 0 | m-D3 | H | H | A-3 | H | H |
| 98 | 0 | m-D3 | H | H | A-4 | H | H |
| 99 | 0 | m-D3 | H | H | A-5 | H | H |
| 100 | 0 | m-D3 | H | H | A-6 | H | H |
| 101 | 0 | m-D3 | H | H | A-7 | H | H |
| 102 | 0 | m-D3 | H | H | A-8 | H | H |
| 103 | 0 | m-D3 | H | H | A-9 | H | H |
| 104 | 0 | m-D3 | H | H | A-10 | H | H |
| 105 | 0 | m-D3 | H | H | A-11 | H | H |
| 106 | 0 | m-D3 | H | H | A-12 | H | H |
| 107 | 0 | m-D3 | H | H | A-13 | H | H |
| 108 | 0 | m-D3 | H | H | A-14 | H | H |
| 109 | 0 | m-D3 | H | H | A-15 | H | H |
| 110 | 0 | m-D3 | H | H | A-16 | H | H |
| 111 | 0 | m-D3 | H | H | A-17 | H | H |
| 112 | 0 | m-D3 | H | H | A-18 | H | H |
| 113 | 0 | m-D3 | H | H | A-19 | H | H |
| 114 | 0 | m-D3 | H | H | A-20 | H | H |
| 115 | 0 | m-D3 | H | H | A-21 | H | H |
| 116 | 0 | m-D3 | H | H | A-22 | H | H |
| 117 | 0 | m-D3 | H | H | A-23 | H | H |
| 118 | 0 | m-D3 | H | H | A-24 | H | H |
| 119 | 0 | m-D3 | H | H | A-25 | H | H |
| 120 | 0 | m-D3 | H | H | A-26 | H | H |
| 121 | 0 | m-D3 | H | H | A-27 | H | H |
| 122 | 0 | m-D3 | H | H | A-28 | H | H |
| 123 | 0 | m-D3 | H | H | A-29 | H | H |
| 124 | 0 | m-D3 | H | H | A-30 | H | H |
| 125 | 0 | m-D3 | H | H | A-31 | H | H |
| 126 | 0 | m-D3 | H | H | A-32 | H | H |
| 127 | 0 | m-D3 | H | H | A-33 | H | H |
| 128 | 0 | m-D3 | H | H | A-34 | H | H |
| 129 | 0 | m-D3 | H | H | A-35 | H | H |
| 130 | 0 | m-D3 | H | H | A-36 | H | H |
| 131 | 0 | m-D3 | H | H | A-37 | H | H |
| 132 | 0 | m-D3 | H | H | A-38 | H | H |
| 133 | 0 | m-D3 | H | H | A-39 | H | H |
| 134 | 0 | m-D3 | H | H | A-40 | H | H |
| 135 | 0 | m-D3 | H | H | A-41 | H | H |
| 136 | 0 | m-D3 | H | H | A-42 | H | H |
| 137 | 0 | m-D3 | H | H | A-43 | H | H |
| 138 | 0 | m-D3 | H | H | A-44 | H | H |
| 139 | 0 | m-D3 | H | H | A-45 | H | H |
| 140 | 0 | m-D3 | H | H | A-46 | H | H |
| 141 | 0 | m-D3 | H | H | A-47 | H | H |
| 142 | 0 | m-D4 | H | H | A-1 | H | H |
| 143 | 0 | m-D4 | H | H | A-2 | H | H |
| 144 | 0 | m-D4 | H | H | A-3 | H | H |
| 145 | 0 | m-D4 | H | H | A-4 | H | H |
| 146 | 0 | m-D4 | H | H | A-5 | H | H |
| 147 | 0 | m-D4 | H | H | A-6 | H | H |
| 148 | 0 | m-D4 | H | H | A-7 | H | H |
| 149 | 0 | m-D4 | H | H | A-8 | H | H |
| 150 | 0 | m-D4 | H | H | A-9 | H | H |
| 151 | 0 | m-D4 | H | H | A-10 | H | H |
| 152 | 0 | m-D4 | H | H | A-11 | H | H |
| 153 | 0 | m-D4 | H | H | A-12 | H | H |
| 154 | 0 | m-D4 | H | H | A-13 | H | H |
| 155 | 0 | m-D4 | H | H | A-14 | H | H |
| 156 | 0 | m-D4 | H | H | A-15 | H | H |
| 157 | 0 | m-D4 | H | H | A-16 | H | H |
| 158 | 0 | m-D4 | H | H | A-17 | H | H |
| 159 | 0 | m-D4 | H | H | A-18 | H | H |
| 160 | 0 | m-D4 | H | H | A-19 | H | H |
| 161 | 0 | m-D4 | H | H | A-20 | H | H |
| 162 | 0 | m-D4 | H | H | A-21 | H | H |
| 163 | 0 | m-D4 | H | H | A-22 | H | H |
| 164 | 0 | m-D4 | H | H | A-23 | H | H |
| 165 | 0 | m-D4 | H | H | A-24 | H | H |
| 166 | 0 | m-D4 | H | H | A-25 | H | H |
| 167 | 0 | m-D4 | H | H | A-26 | H | H |
| 168 | 0 | m-D4 | H | H | A-27 | H | H |
| 169 | 0 | m-D4 | H | H | A-28 | H | H |
| 170 | 0 | m-D4 | H | H | A-29 | H | H |
| 171 | 0 | m-D4 | H | H | A-30 | H | H |
| 172 | 0 | m-D4 | H | H | A-31 | H | H |
| 173 | 0 | m-D4 | H | H | A-32 | H | H |
| 174 | 0 | m-D4 | H | H | A-33 | H | H |
| 175 | 0 | m-D4 | H | H | A-34 | H | H |
| 176 | 0 | m-D4 | H | H | A-35 | H | H |
| 177 | 0 | m-D4 | H | H | A-36 | H | H |
| 178 | 0 | m-D4 | H | H | A-37 | H | H |
| 179 | 0 | m-D4 | H | H | A-38 | H | H |
| 180 | 0 | m-D4 | H | H | A-39 | H | H |
| 181 | 0 | m-D4 | H | H | A-40 | H | H |
| 182 | 0 | m-D4 | H | H | A-41 | H | H |
| 183 | 0 | m-D4 | H | H | A-42 | H | H |
| 184 | 0 | m-D4 | H | H | A-43 | H | H |
| 185 | 0 | m-D4 | H | H | A-44 | H | H |
| 186 | 0 | m-D4 | H | H | A-45 | H | H |
| 187 | 0 | m-D4 | H | H | A-46 | H | H |
| 188 | 0 | m-D4 | H | H | A-47 | H | H |
| 189 | 0 | m-D5 | H | H | A-1 | H | H |
| 190 | 0 | m-D5 | H | H | A-2 | H | H |
| 191 | 0 | m-D5 | H | H | A-3 | H | H |
| 192 | 0 | m-D5 | H | H | A-4 | H | H |
| 193 | 0 | m-D5 | H | H | A-5 | H | H |
| 194 | 0 | m-D5 | H | H | A-6 | H | H |
| 195 | 0 | m-D5 | H | H | A-7 | H | H |
| 196 | 0 | m-D5 | H | H | A-8 | H | H |
| 197 | 0 | m-D5 | H | H | A-9 | H | H |
| 198 | 0 | m-D5 | H | H | A-10 | H | H |
| 199 | 0 | m-D5 | H | H | A-11 | H | H |
| 200 | 0 | m-D5 | H | H | A-12 | H | H |
| 201 | 0 | m-D5 | H | H | A-13 | H | H |
| 202 | 0 | m-D5 | H | H | A-14 | H | H |
| 203 | 0 | m-D5 | H | H | A-15 | H | H |
| 204 | 0 | m-D5 | H | H | A-16 | H | H |
| 205 | 0 | m-D5 | H | H | A-17 | H | H |
| 206 | 0 | m-D5 | H | H | A-18 | H | H |
| 207 | 0 | m-D5 | H | H | A-19 | H | H |
| 208 | 0 | m-D5 | H | H | A-20 | H | H |
| 209 | 0 | m-D5 | H | H | A-21 | H | H |
| 210 | 0 | m-D5 | H | H | A-22 | H | H |
| 211 | 0 | m-D5 | H | H | A-23 | H | H |
| 212 | 0 | m-D5 | H | H | A-24 | H | H |
| 213 | 0 | m-D5 | H | H | A-25 | H | H |
| 214 | 0 | m-D5 | H | H | A-26 | H | H |
| 215 | 0 | m-D5 | H | H | A-27 | H | H |
| 216 | 0 | m-D5 | H | H | A-28 | H | H |
| 217 | 0 | m-D5 | H | H | A-29 | H | H |
| 218 | 0 | m-D5 | H | H | A-30 | H | H |
| 219 | 0 | m-D5 | H | H | A-31 | H | H |
| 220 | 0 | m-D5 | H | H | A-32 | H | H |
| 221 | 0 | m-D5 | H | H | A-33 | H | H |
| 222 | 0 | m-D5 | H | H | A-34 | H | H |
| 223 | 0 | m-D5 | H | H | A-35 | H | H |
| 224 | 0 | m-D5 | H | H | A-36 | H | H |
| 225 | 0 | m-D5 | H | H | A-37 | H | H |
| 226 | 0 | m-D5 | H | H | A-38 | H | H |
| 227 | 0 | m-D5 | H | H | A-39 | H | H |
| 228 | 0 | m-D5 | H | H | A-40 | H | H |
| 229 | 0 | m-D5 | H | H | A-41 | H | H |
| 230 | 0 | m-D5 | H | H | A-42 | H | H |
| 231 | 0 | m-D5 | H | H | A-43 | H | H |
| 232 | 0 | m-D5 | H | H | A-44 | H | H |
| 233 | 0 | m-D5 | H | H | A-45 | H | H |
| 234 | 0 | m-D5 | H | H | A-46 | H | H |
| 235 | 0 | m-D5 | H | H | A-47 | H | H |
| 236 | 0 | m-D6 | H | H | A-1 | H | H |
| 237 | 0 | m-D6 | H | H | A-2 | H | H |
| 238 | 0 | m-D6 | H | H | A-3 | H | H |
| 239 | 0 | m-D6 | H | H | A-4 | H | H |
| 240 | 0 | m-D6 | H | H | A-5 | H | H |
| 241 | 0 | m-D6 | H | H | A-6 | H | H |
| 242 | 0 | m-D6 | H | H | A-7 | H | H |
| 243 | 0 | m-D6 | H | H | A-8 | H | H |
| 244 | 0 | m-D6 | H | H | A-9 | H | H |
| 245 | 0 | m-D6 | H | H | A-10 | H | H |
| 246 | 0 | m-D6 | H | H | A-11 | H | H |
| 247 | 0 | m-D6 | H | H | A-12 | H | H |
| 248 | 0 | m-D6 | H | H | A-13 | H | H |
| 249 | 0 | m-D6 | H | H | A-14 | H | H |
| 250 | 0 | m-D6 | H | H | A-15 | H | H |
| 251 | 0 | m-D6 | H | H | A-16 | H | H |
| 252 | 0 | m-D6 | H | H | A-17 | H | H |
| 253 | 0 | m-D6 | H | H | A-18 | H | H |
| 254 | 0 | m-D6 | H | H | A-19 | H | H |
| 255 | 0 | m-D6 | H | H | A-20 | H | H |
| 256 | 0 | m-D6 | H | H | A-21 | H | H |
| 257 | 0 | m-D6 | H | H | A-22 | H | H |
| 258 | 0 | m-D6 | H | H | A-23 | H | H |
| 259 | 0 | m-D6 | H | H | A-24 | H | H |
| 260 | 0 | m-D6 | H | H | A-25 | H | H |
| 261 | 0 | m-D6 | H | H | A-26 | H | H |
| 262 | 0 | m-D6 | H | H | A-27 | H | H |
| 263 | 0 | m-D6 | H | H | A-28 | H | H |
| 264 | 0 | m-D6 | H | H | A-29 | H | H |
| 265 | 0 | m-D6 | H | H | A-30 | H | H |
| 266 | 0 | m-D6 | H | H | A-31 | H | H |
| 267 | 0 | m-D6 | H | H | A-32 | H | H |
| 268 | 0 | m-D6 | H | H | A-33 | H | H |
| 269 | 0 | m-D6 | H | H | A-34 | H | H |
| 270 | 0 | m-D6 | H | H | A-35 | H | H |
| 271 | 0 | m-D6 | H | H | A-36 | H | H |
| 272 | 0 | m-D6 | H | H | A-37 | H | H |
| 273 | 0 | m-D6 | H | H | A-38 | H | H |
| 274 | 0 | m-D6 | H | H | A-39 | H | H |
| 275 | 0 | m-D6 | H | H | A-40 | H | H |
| 276 | 0 | m-D6 | H | H | A-41 | H | H |
| 277 | 0 | m-D6 | H | H | A-42 | H | H |
| 278 | 0 | m-D6 | H | H | A-43 | H | H |
| 279 | 0 | m-D6 | H | H | A-44 | H | H |
| 280 | 0 | m-D6 | H | H | A-45 | H | H |
| 281 | 0 | m-D6 | H | H | A-46 | H | H |
| 282 | 0 | m-D6 | H | H | A-47 | H | H |
| 283 | 0 | m-D7 | H | H | A-1 | H | H |
| 284 | 0 | m-D7 | H | H | A-2 | H | H |
| 285 | 0 | m-D7 | H | H | A-3 | H | H |
| 286 | 0 | m-D7 | H | H | A-4 | H | H |
| 287 | 0 | m-D7 | H | H | A-5 | H | H |
| 288 | 0 | m-D7 | H | H | A-6 | H | H |
| 289 | 0 | m-D7 | H | H | A-7 | H | H |
| 290 | 0 | m-D7 | H | H | A-8 | H | H |
| 291 | 0 | m-D7 | H | H | A-9 | H | H |
| 292 | 0 | m-D7 | H | H | A-10 | H | H |
| 293 | 0 | m-D7 | H | H | A-11 | H | H |
| 294 | 0 | m-D7 | H | H | A-12 | H | H |
| 295 | 0 | m-D7 | H | H | A-13 | H | H |
| 296 | 0 | m-D7 | H | H | A-14 | H | H |
| 297 | 0 | m-D7 | H | H | A-15 | H | H |
| 298 | 0 | m-D7 | H | H | A-16 | H | H |
| 299 | 0 | m-D7 | H | H | A-17 | H | H |
| 300 | 0 | m-D7 | H | H | A-18 | H | H |
| 301 | 0 | m-D7 | H | H | A-19 | H | H |
| 302 | 0 | m-D7 | H | H | A-20 | H | H |
| 303 | 0 | m-D7 | H | H | A-21 | H | H |
| 304 | 0 | m-D7 | H | H | A-22 | H | H |
| 305 | 0 | m-D7 | H | H | A-23 | H | H |
| 306 | 0 | m-D7 | H | H | A-24 | H | H |
| 307 | 0 | m-D7 | H | H | A-25 | H | H |
| 308 | 0 | m-D7 | H | H | A-26 | H | H |
| 309 | 0 | m-D7 | H | H | A-27 | H | H |
| 310 | 0 | m-D7 | H | H | A-28 | H | H |
| 311 | 0 | m-D7 | H | H | A-29 | H | H |
| 312 | 0 | m-D7 | H | H | A-30 | H | H |
| 313 | 0 | m-D7 | H | H | A-31 | H | H |
| 314 | 0 | m-D7 | H | H | A-32 | H | H |
| 315 | 0 | m-D7 | H | H | A-33 | H | H |
| 316 | 0 | m-D7 | H | H | A-34 | H | H |
| 317 | 0 | m-D7 | H | H | A-35 | H | H |
| 318 | 0 | m-D7 | H | H | A-36 | H | H |
| 319 | 0 | m-D7 | H | H | A-37 | H | H |
| 320 | 0 | m-D7 | H | H | A-38 | H | H |
| 321 | 0 | m-D7 | H | H | A-39 | H | H |
| 322 | 0 | m-D7 | H | H | A-40 | H | H |
| 323 | 0 | m-D7 | H | H | A-41 | H | H |
| 324 | 0 | m-D7 | H | H | A-42 | H | H |
| 325 | 0 | m-D7 | H | H | A-43 | H | H |
| 326 | 0 | m-D7 | H | H | A-44 | H | H |
| 327 | 0 | m-D7 | H | H | A-45 | H | H |
| 328 | 0 | m-D7 | H | H | A-46 | H | H |
| 329 | 0 | m-D7 | H | H | A-47 | H | H |
| 330 | 0 | m-D8 | H | H | A-1 | H | H |
| 331 | 0 | m-D8 | H | H | A-2 | H | H |
| 332 | 0 | m-D8 | H | H | A-3 | H | H |
| 333 | 0 | m-D8 | H | H | A-4 | H | H |
| 334 | 0 | m-D8 | H | H | A-5 | H | H |
| 335 | 0 | m-D8 | H | H | A-6 | H | H |
| 336 | 0 | m-D8 | H | H | A-7 | H | H |
| 337 | 0 | m-D8 | H | H | A-8 | H | H |
| 338 | 0 | m-D8 | H | H | A-9 | H | H |
| 339 | 0 | m-D8 | H | H | A-10 | H | H |
| 340 | 0 | m-D8 | H | H | A-11 | H | H |
| 341 | 0 | m-D8 | H | H | A-12 | H | H |
| 342 | 0 | m-D8 | H | H | A-13 | H | H |
| 343 | 0 | m-D8 | H | H | A-14 | H | H |
| 344 | 0 | m-D8 | H | H | A-15 | H | H |
| 345 | 0 | m-D8 | H | H | A-16 | H | H |
| 346 | 0 | m-D8 | H | H | A-17 | H | H |
| 347 | 0 | m-D8 | H | H | A-18 | H | H |
| 348 | 0 | m-D8 | H | H | A-19 | H | H |
| 349 | 0 | m-D8 | H | H | A-20 | H | H |
| 350 | 0 | m-D8 | H | H | A-21 | H | H |
| 351 | 0 | m-D8 | H | H | A-22 | H | H |
| 352 | 0 | m-D8 | H | H | A-23 | H | H |
| 353 | 0 | m-D8 | H | H | A-24 | H | H |
| 354 | 0 | m-D8 | H | H | A-25 | H | H |
| 355 | 0 | m-D8 | H | H | A-26 | H | H |
| 356 | 0 | m-D8 | H | H | A-27 | H | H |
| 357 | 0 | m-D8 | H | H | A-28 | H | H |
| 358 | 0 | m-D8 | H | H | A-29 | H | H |
| 359 | 0 | m-D8 | H | H | A-30 | H | H |
| 360 | 0 | m-D8 | H | H | A-31 | H | H |
| 361 | 0 | m-D8 | H | H | A-32 | H | H |
| 362 | 0 | m-D8 | H | H | A-33 | H | H |
| 363 | 0 | m-D8 | H | H | A-34 | H | H |
| 364 | 0 | m-D8 | H | H | A-35 | H | H |
| 365 | 0 | m-D8 | H | H | A-36 | H | H |
| 366 | 0 | m-D8 | H | H | A-37 | H | H |
| 367 | 0 | m-D8 | H | H | A-38 | H | H |
| 368 | 0 | m-D8 | H | H | A-39 | H | H |
| 369 | 0 | m-D8 | H | H | A-40 | H | H |
| 370 | 0 | m-D8 | H | H | A-41 | H | H |
| 371 | 0 | m-D8 | H | H | A-42 | H | H |
| 372 | 0 | m-D8 | H | H | A-43 | H | H |
| 373 | 0 | m-D8 | H | H | A-44 | H | H |
| 374 | 0 | m-D8 | H | H | A-45 | H | H |
| 375 | 0 | m-D8 | H | H | A-46 | H | H |
| 376 | 0 | m-D8 | H | H | A-47 | H | H |
| 377 | 0 | m-D9 | H | H | A-1 | H | H |
| 378 | 0 | m-D9 | H | H | A-2 | H | H |
| 379 | 0 | m-D9 | H | H | A-3 | H | H |
| 380 | 0 | m-D9 | H | H | A-4 | H | H |
| 381 | 0 | m-D9 | H | H | A-5 | H | H |
| 382 | 0 | m-D9 | H | H | A-6 | H | H |
| 383 | 0 | m-D9 | H | H | A-7 | H | H |
| 384 | 0 | m-D9 | H | H | A-8 | H | H |
| 385 | 0 | m-D9 | H | H | A-9 | H | H |
| 386 | 0 | m-D9 | H | H | A-10 | H | H |
| 387 | 0 | m-D9 | H | H | A-11 | H | H |
| 388 | 0 | m-D9 | H | H | A-12 | H | H |
| 389 | 0 | m-D9 | H | H | A-13 | H | H |
| 390 | 0 | m-D9 | H | H | A-14 | H | H |
| 391 | 0 | m-D9 | H | H | A-15 | H | H |
| 392 | 0 | m-D9 | H | H | A-16 | H | H |
| 393 | 0 | m-D9 | H | H | A-17 | H | H |
| 394 | 0 | m-D9 | H | H | A-18 | H | H |
| 395 | 0 | m-D9 | H | H | A-19 | H | H |
| 396 | 0 | m-D9 | H | H | A-20 | H | H |
| 397 | 0 | m-D9 | H | H | A-21 | H | H |
| 398 | 0 | m-D9 | H | H | A-22 | H | H |
| 399 | 0 | m-D9 | H | H | A-23 | H | H |
| 400 | 0 | m-D9 | H | H | A-24 | H | H |
| 401 | 0 | m-D9 | H | H | A-25 | H | H |
| 402 | 0 | m-D9 | H | H | A-26 | H | H |
| 403 | 0 | m-D9 | H | H | A-27 | H | H |
| 404 | 0 | m-D9 | H | H | A-28 | H | H |
| 405 | 0 | m-D9 | H | H | A-29 | H | H |
| 406 | 0 | m-D9 | H | H | A-30 | H | H |
| 407 | 0 | m-D9 | H | H | A-31 | H | H |
| 408 | 0 | m-D9 | H | H | A-32 | H | H |
| 409 | 0 | m-D9 | H | H | A-33 | H | H |
| 410 | 0 | m-D9 | H | H | A-34 | H | H |
| 411 | 0 | m-D9 | H | H | A-35 | H | H |
| 412 | 0 | m-D9 | H | H | A-36 | H | H |
| 413 | 0 | m-D9 | H | H | A-37 | H | H |
| 414 | 0 | m-D9 | H | H | A-38 | H | H |
| 415 | 0 | m-D9 | H | H | A-39 | H | H |
| 416 | 0 | m-D9 | H | H | A-40 | H | H |
| 417 | 0 | m-D9 | H | H | A-41 | H | H |
| 418 | 0 | m-D9 | H | H | A-42 | H | H |
| 419 | 0 | m-D9 | H | H | A-43 | H | H |
| 420 | 0 | m-D9 | H | H | A-44 | H | H |
| 421 | 0 | m-D9 | H | H | A-45 | H | H |
| 422 | 0 | m-D9 | H | H | A-46 | H | H |
| 423 | 0 | m-D9 | H | H | A-47 | H | H |
| 424 | 0 | m-D10 | H | H | A-1 | H | H |
| 425 | 0 | m-D10 | H | H | A-2 | H | H |
| 426 | 0 | m-D10 | H | H | A-3 | H | H |
| 427 | 0 | m-D10 | H | H | A-4 | H | H |
| 428 | 0 | m-D10 | H | H | A-5 | H | H |
| 429 | 0 | m-D10 | H | H | A-6 | H | H |
| 430 | 0 | m-D10 | H | H | A-7 | H | H |
| 431 | 0 | m-D10 | H | H | A-8 | H | H |
| 432 | 0 | m-D10 | H | H | A-9 | H | H |
| 433 | 0 | m-D10 | H | H | A-10 | H | H |
| 434 | 0 | m-D10 | H | H | A-11 | H | H |
| 435 | 0 | m-D10 | H | H | A-12 | H | H |
| 436 | 0 | m-D10 | H | H | A-13 | H | H |
| 437 | 0 | m-D10 | H | H | A-14 | H | H |
| 438 | 0 | m-D10 | H | H | A-15 | H | H |
| 439 | 0 | m-D10 | H | H | A-16 | H | H |
| 440 | 0 | m-D10 | H | H | A-17 | H | H |
| 441 | 0 | m-D10 | H | H | A-18 | H | H |
| 442 | 0 | m-D10 | H | H | A-19 | H | H |
| 443 | 0 | m-D10 | H | H | A-20 | H | H |
| 444 | 0 | m-D10 | H | H | A-21 | H | H |
| 445 | 0 | m-D10 | H | H | A-22 | H | H |
| 446 | 0 | m-D10 | H | H | A-23 | H | H |
| 447 | 0 | m-D10 | H | H | A-24 | H | H |
| 448 | 0 | m-D10 | H | H | A-25 | H | H |
| 449 | 0 | m-D10 | H | H | A-26 | H | H |
| 450 | 0 | m-D10 | H | H | A-27 | H | H |
| 451 | 0 | m-D10 | H | H | A-28 | H | H |
| 452 | 0 | m-D10 | H | H | A-29 | H | H |
| 453 | 0 | m-D10 | H | H | A-30 | H | H |
| 454 | 0 | m-D10 | H | H | A-31 | H | H |
| 455 | 0 | m-D10 | H | H | A-32 | H | H |
| 456 | 0 | m-D10 | H | H | A-33 | H | H |
| 457 | 0 | m-D10 | H | H | A-34 | H | H |
| 458 | 0 | m-D10 | H | H | A-35 | H | H |
| 459 | 0 | m-D10 | H | H | A-36 | H | H |
| 460 | 0 | m-D10 | H | H | A-37 | H | H |
| 461 | 0 | m-D10 | H | H | A-38 | H | H |
| 462 | 0 | m-D10 | H | H | A-39 | H | H |
| 463 | 0 | m-D10 | H | H | A-40 | H | H |
| 464 | 0 | m-D10 | H | H | A-41 | H | H |
| 465 | 0 | m-D10 | H | H | A-42 | H | H |
| 466 | 0 | m-D10 | H | H | A-43 | H | H |
| 467 | 0 | m-D10 | H | H | A-44 | H | H |
| 468 | 0 | m-D10 | H | H | A-45 | H | H |
| 469 | 0 | m-D10 | H | H | A-46 | H | H |
| 470 | 0 | m-D10 | H | H | A-47 | H | H |
| 471 | 0 | m-D11 | H | H | A-1 | H | H |
| 472 | 0 | m-D11 | H | H | A-2 | H | H |
| 473 | 0 | m-D11 | H | H | A-3 | H | H |
| 474 | 0 | m-D11 | H | H | A-4 | H | H |
| 475 | 0 | m-D11 | H | H | A-5 | H | H |
| 476 | 0 | m-D11 | H | H | A-6 | H | H |
| 477 | 0 | m-D11 | H | H | A-7 | H | H |
| 478 | 0 | m-D11 | H | H | A-8 | H | H |
| 479 | 0 | m-D11 | H | H | A-9 | H | H |
| 480 | 0 | m-D11 | H | H | A-10 | H | H |
| 481 | 0 | m-D11 | H | H | A-11 | H | H |
| 482 | 0 | m-D11 | H | H | A-12 | H | H |
| 483 | 0 | m-D11 | H | H | A-13 | H | H |
| 484 | 0 | m-D11 | H | H | A-14 | H | H |
| 485 | 0 | m-D11 | H | H | A-15 | H | H |
| 486 | 0 | m-D11 | H | H | A-16 | H | H |
| 487 | 0 | m-D11 | H | H | A-17 | H | H |
| 488 | 0 | m-D11 | H | H | A-18 | H | H |
| 489 | 0 | m-D11 | H | H | A-19 | H | H |
| 490 | 0 | m-D11 | H | H | A-20 | H | H |
| 491 | 0 | m-D11 | H | H | A-21 | H | H |
| 492 | 0 | m-D11 | H | H | A-22 | H | H |
| 493 | 0 | m-D11 | H | H | A-23 | H | H |
| 494 | 0 | m-D11 | H | H | A-24 | H | H |
| 495 | 0 | m-D11 | H | H | A-25 | H | H |
| 496 | 0 | m-D11 | H | H | A-26 | H | H |
| 497 | 0 | m-D11 | H | H | A-27 | H | H |
| 498 | 0 | m-D11 | H | H | A-28 | H | H |
| 499 | 0 | m-D11 | H | H | A-29 | H | H |
| 500 | 0 | m-D11 | H | H | A-30 | H | H |
| 501 | 0 | m-D11 | H | H | A-31 | H | H |
| 502 | 0 | m-D11 | H | H | A-32 | H | H |
| 503 | 0 | m-D11 | H | H | A-33 | H | H |
| 504 | 0 | m-D11 | H | H | A-34 | H | H |
| 505 | 0 | m-D11 | H | H | A-35 | H | H |
| 506 | 0 | m-D11 | H | H | A-36 | H | H |
| 507 | 0 | m-D11 | H | H | A-37 | H | H |
| 508 | 0 | m-D11 | H | H | A-38 | H | H |
| 509 | 0 | m-D11 | H | H | A-39 | H | H |
| 510 | 0 | m-D11 | H | H | A-40 | H | H |
| 511 | 0 | m-D11 | H | H | A-41 | H | H |
| 512 | 0 | m-D11 | H | H | A-42 | H | H |
| 513 | 0 | m-D11 | H | H | A-43 | H | H |
| 514 | 0 | m-D11 | H | H | A-44 | H | H |
| 515 | 0 | m-D11 | H | H | A-45 | H | H |
| 516 | 0 | m-D11 | H | H | A-46 | H | H |
| 517 | 0 | m-D11 | H | H | A-47 | H | H |
| 518 | 0 | m-D12 | H | H | A-1 | H | H |
| 519 | 0 | m-D12 | H | H | A-2 | H | H |
| 520 | 0 | m-D12 | H | H | A-3 | H | H |
| 521 | 0 | m-D12 | H | H | A-4 | H | H |
| 522 | 0 | m-D12 | H | H | A-5 | H | H |
| 523 | 0 | m-D12 | H | H | A-6 | H | H |
| 524 | 0 | m-D12 | H | H | A-7 | H | H |
| 525 | 0 | m-D12 | H | H | A-8 | H | H |
| 526 | 0 | m-D12 | H | H | A-9 | H | H |
| 527 | 0 | m-D12 | H | H | A-10 | H | H |
| 528 | 0 | m-D12 | H | H | A-11 | H | H |
| 529 | 0 | m-D12 | H | H | A-12 | H | H |
| 530 | 0 | m-D12 | H | H | A-13 | H | H |
| 531 | 0 | m-D12 | H | H | A-14 | H | H |
| 532 | 0 | m-D12 | H | H | A-15 | H | H |
| 533 | 0 | m-D12 | H | H | A-16 | H | H |
| 534 | 0 | m-D12 | H | H | A-17 | H | H |
| 535 | 0 | m-D12 | H | H | A-18 | H | H |
| 536 | 0 | m-D12 | H | H | A-19 | H | H |
| 537 | 0 | m-D12 | H | H | A-20 | H | H |
| 538 | 0 | m-D12 | H | H | A-21 | H | H |
| 539 | 0 | m-D12 | H | H | A-22 | H | H |
| 540 | 0 | m-D12 | H | H | A-23 | H | H |
| 541 | 0 | m-D12 | H | H | A-24 | H | H |
| 542 | 0 | m-D12 | H | H | A-25 | H | H |
| 543 | 0 | m-D12 | H | H | A-26 | H | H |
| 544 | 0 | m-D12 | H | H | A-27 | H | H |
| 545 | 0 | m-D12 | H | H | A-28 | H | H |
| 546 | 0 | m-D12 | H | H | A-29 | H | H |
| 547 | 0 | m-D12 | H | H | A-30 | H | H |
| 548 | 0 | m-D12 | H | H | A-31 | H | H |
| 549 | 0 | m-D12 | H | H | A-32 | H | H |
| 550 | 0 | m-D12 | H | H | A-33 | H | H |
| 551 | 0 | m-D12 | H | H | A-34 | H | H |
| 552 | 0 | m-D12 | H | H | A-35 | H | H |
| 553 | 0 | m-D12 | H | H | A-36 | H | H |
| 554 | 0 | m-D12 | H | H | A-37 | H | H |
| 555 | 0 | m-D12 | H | H | A-38 | H | H |
| 556 | 0 | m-D12 | H | H | A-39 | H | H |
| 557 | 0 | m-D12 | H | H | A-40 | H | H |
| 558 | 0 | m-D12 | H | H | A-41 | H | H |
| 559 | 0 | m-D12 | H | H | A-42 | H | H |
| 560 | 0 | m-D12 | H | H | A-43 | H | H |
| 561 | 0 | m-D12 | H | H | A-44 | H | H |
| 562 | 0 | m-D12 | H | H | A-45 | H | H |
| 563 | 0 | m-D12 | H | H | A-46 | H | H |
| 564 | 0 | m-D12 | H | H | A-47 | H | H |
| 565 | 0 | m-D13 | H | H | A-1 | H | H |
| 566 | 0 | m-D13 | H | H | A-2 | H | H |
| 567 | 0 | m-D13 | H | H | A-3 | H | H |
| 568 | 0 | m-D13 | H | H | A-4 | H | H |
| 569 | 0 | m-D13 | H | H | A-5 | H | H |
| 570 | 0 | m-D13 | H | H | A-6 | H | H |
| 571 | 0 | m-D13 | H | H | A-7 | H | H |
| 572 | 0 | m-D13 | H | H | A-8 | H | H |
| 573 | 0 | m-D13 | H | H | A-9 | H | H |
| 574 | 0 | m-D13 | H | H | A-10 | H | H |
| 575 | 0 | m-D13 | H | H | A-11 | H | H |
| 576 | 0 | m-D13 | H | H | A-12 | H | H |
| 577 | 0 | m-D13 | H | H | A-13 | H | H |
| 578 | 0 | m-D13 | H | H | A-14 | H | H |
| 579 | 0 | m-D13 | H | H | A-15 | H | H |
| 580 | 0 | m-D13 | H | H | A-16 | H | H |
| 581 | 0 | m-D13 | H | H | A-17 | H | H |
| 582 | 0 | m-D13 | H | H | A-18 | H | H |
| 583 | 0 | m-D13 | H | H | A-19 | H | H |
| 584 | 0 | m-D13 | H | H | A-20 | H | H |
| 585 | 0 | m-D13 | H | H | A-21 | H | H |
| 586 | 0 | m-D13 | H | H | A-22 | H | H |
| 587 | 0 | m-D13 | H | H | A-23 | H | H |
| 588 | 0 | m-D13 | H | H | A-24 | H | H |
| 589 | 0 | m-D13 | H | H | A-25 | H | H |
| 590 | 0 | m-D13 | H | H | A-26 | H | H |
| 591 | 0 | m-D13 | H | H | A-27 | H | H |
| 592 | 0 | m-D13 | H | H | A-28 | H | H |
| 593 | 0 | m-D13 | H | H | A-29 | H | H |
| 594 | 0 | m-D13 | H | H | A-30 | H | H |
| 595 | 0 | m-D13 | H | H | A-31 | H | H |
| 596 | 0 | m-D13 | H | H | A-32 | H | H |
| 597 | 0 | m-D13 | H | H | A-33 | H | H |
| 598 | 0 | m-D13 | H | H | A-34 | H | H |
| 599 | 0 | m-D13 | H | H | A-35 | H | H |
| 600 | 0 | m-D13 | H | H | A-36 | H | H |
| 601 | 0 | m-D13 | H | H | A-37 | H | H |
| 602 | 0 | m-D13 | H | H | A-38 | H | H |
| 603 | 0 | m-D13 | H | H | A-39 | H | H |
| 604 | 0 | m-D13 | H | H | A-40 | H | H |
| 605 | 0 | m-D13 | H | H | A-41 | H | H |
| 606 | 0 | m-D13 | H | H | A-42 | H | H |
| 607 | 0 | m-D13 | H | H | A-43 | H | H |
| 608 | 0 | m-D13 | H | H | A-44 | H | H |
| 609 | 0 | m-D13 | H | H | A-45 | H | H |
| 610 | 0 | m-D13 | H | H | A-46 | H | H |
| 611 | 0 | m-D13 | H | H | A-47 | H | H |
| 612 | 0 | m-D14 | H | H | A-1 | H | H |
| 613 | 0 | m-D14 | H | H | A-2 | H | H |
| 614 | 0 | m-D14 | H | H | A-3 | H | H |
| 615 | 0 | m-D14 | H | H | A-4 | H | H |
| 616 | 0 | m-D14 | H | H | A-5 | H | H |
| 617 | 0 | m-D14 | H | H | A-6 | H | H |
| 618 | 0 | m-D14 | H | H | A-7 | H | H |
| 619 | 0 | m-D14 | H | H | A-8 | H | H |
| 620 | 0 | m-D14 | H | H | A-9 | H | H |
| 621 | 0 | m-D14 | H | H | A-10 | H | H |
| 622 | 0 | m-D14 | H | H | A-11 | H | H |
| 623 | 0 | m-D14 | H | H | A-12 | H | H |
| 624 | 0 | m-D14 | H | H | A-13 | H | H |
| 625 | 0 | m-D14 | H | H | A-14 | H | H |
| 626 | 0 | m-D14 | H | H | A-15 | H | H |
| 627 | 0 | m-D14 | H | H | A-16 | H | H |
| 628 | 0 | m-D14 | H | H | A-17 | H | H |
| 629 | 0 | m-D14 | H | H | A-18 | H | H |
| 630 | 0 | m-D14 | H | H | A-19 | H | H |
| 631 | 0 | m-D14 | H | H | A-20 | H | H |
| 632 | 0 | m-D14 | H | H | A-21 | H | H |
| 633 | 0 | m-D14 | H | H | A-22 | H | H |
| 634 | 0 | m-D14 | H | H | A-23 | H | H |
| 635 | 0 | m-D14 | H | H | A-24 | H | H |
| 636 | 0 | m-D14 | H | H | A-25 | H | H |
| 637 | 0 | m-D14 | H | H | A-26 | H | H |
| 638 | 0 | m-D14 | H | H | A-27 | H | H |
| 639 | 0 | m-D14 | H | H | A-28 | H | H |
| 640 | 0 | m-D14 | H | H | A-29 | H | H |
| 641 | 0 | m-D14 | H | H | A-30 | H | H |
| 642 | 0 | m-D14 | H | H | A-31 | H | H |
| 643 | 0 | m-D14 | H | H | A-32 | H | H |
| 644 | 0 | m-D14 | H | H | A-33 | H | H |
| 645 | 0 | m-D14 | H | H | A-34 | H | H |
| 646 | 0 | m-D14 | H | H | A-35 | H | H |
| 647 | 0 | m-D14 | H | H | A-36 | H | H |
| 648 | 0 | m-D14 | H | H | A-37 | H | H |
| 649 | 0 | m-D14 | H | H | A-38 | H | H |
| 650 | 0 | m-D14 | H | H | A-39 | H | H |
| 651 | 0 | m-D14 | H | H | A-40 | H | H |
| 652 | 0 | m-D14 | H | H | A-41 | H | H |
| 653 | 0 | m-D14 | H | H | A-42 | H | H |
| 654 | 0 | m-D14 | H | H | A-43 | H | H |
| 655 | 0 | m-D14 | H | H | A-44 | H | H |
| 656 | 0 | m-D14 | H | H | A-45 | H | H |
| 657 | 0 | m-D14 | H | H | A-46 | H | H |
| 658 | 0 | m-D14 | H | H | A-47 | H | H |
| 659 | 0 | m-D1 | A-1 | H | H | H | H |
| 660 | 0 | m-D1 | A-2 | H | H | H | H |
| 661 | 0 | m-D1 | A-3 | H | H | H | H |
| 662 | 0 | m-D1 | A-4 | H | H | H | H |
| 663 | 0 | m-D1 | A-5 | H | H | H | H |
| 664 | 0 | m-D1 | A-6 | H | H | H | H |
| 665 | 0 | m-D1 | A-7 | H | H | H | H |
| 666 | 0 | m-D1 | A-8 | H | H | H | H |
| 667 | 0 | m-D1 | A-9 | H | H | H | H |
| 668 | 0 | m-D1 | A-10 | H | H | H | H |
| 669 | 0 | m-D1 | A-11 | H | H | H | H |
| 670 | 0 | m-D1 | A-12 | H | H | H | H |
| 671 | 0 | m-D1 | A-13 | H | H | H | H |
| 672 | 0 | m-D1 | A-14 | H | H | H | H |
| 673 | 0 | m-D1 | A-15 | H | H | H | H |
| 674 | 0 | m-D1 | A-16 | H | H | H | H |
| 675 | 0 | m-D1 | A-17 | H | H | H | H |
| 676 | 0 | m-D1 | A-18 | H | H | H | H |
| 677 | 0 | m-D1 | A-19 | H | H | H | H |
| 678 | 0 | m-D1 | A-20 | H | H | H | H |
| 679 | 0 | m-D1 | A-21 | H | H | H | H |
| 680 | 0 | m-D1 | A-22 | H | H | H | H |
| 681 | 0 | m-D1 | A-23 | H | H | H | H |
| 682 | 0 | m-D1 | A-24 | H | H | H | H |
| 683 | 0 | m-D1 | A-25 | H | H | H | H |
| 684 | 0 | m-D1 | A-26 | H | H | H | H |
| 685 | 0 | m-D1 | A-27 | H | H | H | H |
| 686 | 0 | m-D1 | A-28 | H | H | H | H |
| 687 | 0 | m-D1 | A-29 | H | H | H | H |
| 688 | 0 | m-D1 | A-30 | H | H | H | H |
| 689 | 0 | m-D1 | A-31 | H | H | H | H |
| 690 | 0 | m-D1 | A-32 | H | H | H | H |
| 691 | 0 | m-D1 | A-33 | H | H | H | H |
| 692 | 0 | m-D1 | A-34 | H | H | H | H |
| 693 | 0 | m-D1 | A-35 | H | H | H | H |
| 694 | 0 | m-D1 | A-36 | H | H | H | H |
| 695 | 0 | m-D1 | A-37 | H | H | H | H |
| 696 | 0 | m-D1 | A-38 | H | H | H | H |
| 697 | 0 | m-D1 | A-39 | H | H | H | H |
| 698 | 0 | m-D1 | A-40 | H | H | H | H |
| 699 | 0 | m-D1 | A-41 | H | H | H | H |
| 700 | 0 | m-D1 | A-42 | H | H | H | H |
| 701 | 0 | m-D1 | A-43 | H | H | H | H |
| 702 | 0 | m-D1 | A-44 | H | H | H | H |
| 703 | 0 | m-D1 | A-45 | H | H | H | H |
| 704 | 0 | m-D1 | A-46 | H | H | H | H |
| 705 | 0 | m-D1 | A-47 | H | H | H | H |
| 706 | 0 | m-D2 | A-1 | H | H | H | H |
| 707 | 0 | m-D2 | A-2 | H | H | H | H |
| 708 | 0 | m-D2 | A-3 | H | H | H | H |
| 709 | 0 | m-D2 | A-4 | H | H | H | H |
| 710 | 0 | m-D2 | A-5 | H | H | H | H |
| 711 | 0 | m-D2 | A-6 | H | H | H | H |
| 712 | 0 | m-D2 | A-7 | H | H | H | H |
| 713 | 0 | m-D2 | A-8 | H | H | H | H |
| 714 | 0 | m-D2 | A-9 | H | H | H | H |
| 715 | 0 | m-D2 | A-10 | H | H | H | H |
| 716 | 0 | m-D2 | A-11 | H | H | H | H |
| 717 | 0 | m-D2 | A-12 | H | H | H | H |
| 718 | 0 | m-D2 | A-13 | H | H | H | H |
| 719 | 0 | m-D2 | A-14 | H | H | H | H |
| 720 | 0 | m-D2 | A-15 | H | H | H | H |
| 721 | 0 | m-D2 | A-16 | H | H | H | H |
| 722 | 0 | m-D2 | A-17 | H | H | H | H |
| 723 | 0 | m-D2 | A-18 | H | H | H | H |
| 724 | 0 | m-D2 | A-19 | H | H | H | H |
| 725 | 0 | m-D2 | A-20 | H | H | H | H |
| 726 | 0 | m-D2 | A-21 | H | H | H | H |
| 727 | 0 | m-D2 | A-22 | H | H | H | H |
| 728 | 0 | m-D2 | A-23 | H | H | H | H |
| 729 | 0 | m-D2 | A-24 | H | H | H | H |
| 730 | 0 | m-D2 | A-25 | H | H | H | H |
| 731 | 0 | m-D2 | A-26 | H | H | H | H |
| 732 | 0 | m-D2 | A-27 | H | H | H | H |
| 733 | 0 | m-D2 | A-28 | H | H | H | H |
| 734 | 0 | m-D2 | A-29 | H | H | H | H |
| 735 | 0 | m-D2 | A-30 | H | H | H | H |
| 736 | 0 | m-D2 | A-31 | H | H | H | H |
| 737 | 0 | m-D2 | A-32 | H | H | H | H |
| 738 | 0 | m-D2 | A-33 | H | H | H | H |
| 739 | 0 | m-D2 | A-34 | H | H | H | H |
| 740 | 0 | m-D2 | A-35 | H | H | H | H |
| 741 | 0 | m-D2 | A-36 | H | H | H | H |
| 742 | 0 | m-D2 | A-37 | H | H | H | H |
| 743 | 0 | m-D2 | A-38 | H | H | H | H |
| 744 | 0 | m-D2 | A-39 | H | H | H | H |
| 745 | 0 | m-D2 | A-40 | H | H | H | H |
| 746 | 0 | m-D2 | A-41 | H | H | H | H |
| 747 | 0 | m-D2 | A-42 | H | H | H | H |
| 748 | 0 | m-D2 | A-43 | H | H | H | H |
| 749 | 0 | m-D2 | A-44 | H | H | H | H |
| 750 | 0 | m-D2 | A-45 | H | H | H | H |
| 751 | 0 | m-D2 | A-46 | H | H | H | H |
| 752 | 0 | m-D2 | A-47 | H | H | H | H |
| 753 | 0 | m-D3 | A-1 | H | H | H | H |
| 754 | 0 | m-D3 | A-2 | H | H | H | H |
| 755 | 0 | m-D3 | A-3 | H | H | H | H |
| 756 | 0 | m-D3 | A-4 | H | H | H | H |
| 757 | 0 | m-D3 | A-5 | H | H | H | H |
| 758 | 0 | m-D3 | A-6 | H | H | H | H |
| 759 | 0 | m-D3 | A-7 | H | H | H | H |
| 760 | 0 | m-D3 | A-8 | H | H | H | H |
| 761 | 0 | m-D3 | A-9 | H | H | H | H |
| 762 | 0 | m-D3 | A-10 | H | H | H | H |
| 763 | 0 | m-D3 | A-11 | H | H | H | H |
| 764 | 0 | m-D3 | A-12 | H | H | H | H |
| 765 | 0 | m-D3 | A-13 | H | H | H | H |
| 766 | 0 | m-D3 | A-14 | H | H | H | H |
| 767 | 0 | m-D3 | A-15 | H | H | H | H |
| 768 | 0 | m-D3 | A-16 | H | H | H | H |
| 769 | 0 | m-D3 | A-17 | H | H | H | H |
| 770 | 0 | m-D3 | A-18 | H | H | H | H |
| 771 | 0 | m-D3 | A-19 | H | H | H | H |
| 772 | 0 | m-D3 | A-20 | H | H | H | H |
| 773 | 0 | m-D3 | A-21 | H | H | H | H |
| 774 | 0 | m-D3 | A-22 | H | H | H | H |
| 775 | 0 | m-D3 | A-23 | H | H | H | H |
| 776 | 0 | m-D3 | A-24 | H | H | H | H |
| 777 | 0 | m-D3 | A-25 | H | H | H | H |
| 778 | 0 | m-D3 | A-26 | H | H | H | H |
| 779 | 0 | m-D3 | A-27 | H | H | H | H |
| 780 | 0 | m-D3 | A-28 | H | H | H | H |
| 781 | 0 | m-D3 | A-29 | H | H | H | H |
| 782 | 0 | m-D3 | A-30 | H | H | H | H |
| 783 | 0 | m-D3 | A-31 | H | H | H | H |
| 784 | 0 | m-D3 | A-32 | H | H | H | H |
| 785 | 0 | m-D3 | A-33 | H | H | H | H |
| 786 | 0 | m-D3 | A-34 | H | H | H | H |
| 787 | 0 | m-D3 | A-35 | H | H | H | H |
| 788 | 0 | m-D3 | A-36 | H | H | H | H |
| 789 | 0 | m-D3 | A-37 | H | H | H | H |
| 790 | 0 | m-D3 | A-38 | H | H | H | H |
| 791 | 0 | m-D3 | A-39 | H | H | H | H |
| 792 | 0 | m-D3 | A-40 | H | H | H | H |
| 793 | 0 | m-D3 | A-41 | H | H | H | H |
| 794 | 0 | m-D3 | A-42 | H | H | H | H |
| 795 | 0 | m-D3 | A-43 | H | H | H | H |
| 796 | 0 | m-D3 | A-44 | H | H | H | H |
| 797 | 0 | m-D3 | A-45 | H | H | H | H |
| 798 | 0 | m-D3 | A-46 | H | H | H | H |
| 799 | 0 | m-D3 | A-47 | H | H | H | H |
| 800 | 0 | m-D4 | A-1 | H | H | H | H |
| 801 | 0 | m-D4 | A-2 | H | H | H | H |
| 802 | 0 | m-D4 | A-3 | H | H | H | H |
| 803 | 0 | m-D4 | A-4 | H | H | H | H |
| 804 | 0 | m-D4 | A-5 | H | H | H | H |
| 805 | 0 | m-D4 | A-6 | H | H | H | H |
| 806 | 0 | m-D4 | A-7 | H | H | H | H |
| 807 | 0 | m-D4 | A-8 | H | H | H | H |
| 808 | 0 | m-D4 | A-9 | H | H | H | H |
| 809 | 0 | m-D4 | A-10 | H | H | H | H |
| 810 | 0 | m-D4 | A-11 | H | H | H | H |
| 811 | 0 | m-D4 | A-12 | H | H | H | H |
| 812 | 0 | m-D4 | A-13 | H | H | H | H |
| 813 | 0 | m-D4 | A-14 | H | H | H | H |
| 814 | 0 | m-D4 | A-15 | H | H | H | H |
| 815 | 0 | m-D4 | A-16 | H | H | H | H |
| 816 | 0 | m-D4 | A-17 | H | H | H | H |
| 817 | 0 | m-D4 | A-18 | H | H | H | H |
| 818 | 0 | m-D4 | A-19 | H | H | H | H |
| 819 | 0 | m-D4 | A-20 | H | H | H | H |
| 820 | 0 | m-D4 | A-21 | H | H | H | H |
| 821 | 0 | m-D4 | A-22 | H | H | H | H |
| 822 | 0 | m-D4 | A-23 | H | H | H | H |
| 823 | 0 | m-D4 | A-24 | H | H | H | H |
| 824 | 0 | m-D4 | A-25 | H | H | H | H |
| 825 | 0 | m-D4 | A-26 | H | H | H | H |
| 826 | 0 | m-D4 | A-27 | H | H | H | H |
| 827 | 0 | m-D4 | A-28 | H | H | H | H |
| 828 | 0 | m-D4 | A-29 | H | H | H | H |
| 829 | 0 | m-D4 | A-30 | H | H | H | H |
| 830 | 0 | m-D4 | A-31 | H | H | H | H |
| 831 | 0 | m-D4 | A-32 | H | H | H | H |
| 832 | 0 | m-D4 | A-33 | H | H | H | H |
| 833 | 0 | m-D4 | A-34 | H | H | H | H |
| 834 | 0 | m-D4 | A-35 | H | H | H | H |
| 835 | 0 | m-D4 | A-36 | H | H | H | H |
| 836 | 0 | m-D4 | A-37 | H | H | H | H |
| 837 | 0 | m-D4 | A-38 | H | H | H | H |
| 838 | 0 | m-D4 | A-39 | H | H | H | H |
| 839 | 0 | m-D4 | A-40 | H | H | H | H |
| 840 | 0 | m-D4 | A-41 | H | H | H | H |
| 841 | 0 | m-D4 | A-42 | H | H | H | H |
| 842 | 0 | m-D4 | A-43 | H | H | H | H |
| 843 | 0 | m-D4 | A-44 | H | H | H | H |
| 844 | 0 | m-D4 | A-45 | H | H | H | H |
| 845 | 0 | m-D4 | A-46 | H | H | H | H |
| 846 | 0 | m-D4 | A-47 | H | H | H | H |
| 847 | 0 | m-D5 | A-1 | H | H | H | H |
| 848 | 0 | m-D5 | A-2 | H | H | H | H |
| 849 | 0 | m-D5 | A-3 | H | H | H | H |
| 850 | 0 | m-D5 | A-4 | H | H | H | H |
| 851 | 0 | m-D5 | A-5 | H | H | H | H |
| 852 | 0 | m-D5 | A-6 | H | H | H | H |
| 853 | 0 | m-D5 | A-7 | H | H | H | H |
| 854 | 0 | m-D5 | A-8 | H | H | H | H |
| 855 | 0 | m-D5 | A-9 | H | H | H | H |
| 856 | 0 | m-D5 | A-10 | H | H | H | H |
| 857 | 0 | m-D5 | A-11 | H | H | H | H |
| 858 | 0 | m-D5 | A-12 | H | H | H | H |
| 859 | 0 | m-D5 | A-13 | H | H | H | H |
| 860 | 0 | m-D5 | A-14 | H | H | H | H |
| 861 | 0 | m-D5 | A-15 | H | H | H | H |
| 862 | 0 | m-D5 | A-16 | H | H | H | H |
| 863 | 0 | m-D5 | A-17 | H | H | H | H |
| 864 | 0 | m-D5 | A-18 | H | H | H | H |
| 865 | 0 | m-D5 | A-19 | H | H | H | H |
| 866 | 0 | m-D5 | A-20 | H | H | H | H |
| 867 | 0 | m-D5 | A-21 | H | H | H | H |
| 868 | 0 | m-D5 | A-22 | H | H | H | H |
| 869 | 0 | m-D5 | A-23 | H | H | H | H |
| 870 | 0 | m-D5 | A-24 | H | H | H | H |
| 871 | 0 | m-D5 | A-25 | H | H | H | H |
| 872 | 0 | m-D5 | A-26 | H | H | H | H |
| 873 | 0 | m-D5 | A-27 | H | H | H | H |
| 874 | 0 | m-D5 | A-28 | H | H | H | H |
| 875 | 0 | m-D5 | A-29 | H | H | H | H |
| 876 | 0 | m-D5 | A-30 | H | H | H | H |
| 877 | 0 | m-D5 | A-31 | H | H | H | H |
| 878 | 0 | m-D5 | A-32 | H | H | H | H |
| 879 | 0 | m-D5 | A-33 | H | H | H | H |
| 880 | 0 | m-D5 | A-34 | H | H | H | H |
| 881 | 0 | m-D5 | A-35 | H | H | H | H |
| 882 | 0 | m-D5 | A-36 | H | H | H | H |
| 883 | 0 | m-D5 | A-37 | H | H | H | H |
| 884 | 0 | m-D5 | A-38 | H | H | H | H |
| 885 | 0 | m-D5 | A-39 | H | H | H | H |
| 886 | 0 | m-D5 | A-40 | H | H | H | H |
| 887 | 0 | m-D5 | A-41 | H | H | H | H |
| 888 | 0 | m-D5 | A-42 | H | H | H | H |
| 889 | 0 | m-D5 | A-43 | H | H | H | H |
| 890 | 0 | m-D5 | A-44 | H | H | H | H |
| 891 | 0 | m-D5 | A-45 | H | H | H | H |
| 892 | 0 | m-D5 | A-46 | H | H | H | H |
| 893 | 0 | m-D5 | A-47 | H | H | H | H |
| 894 | 0 | m-D6 | A-1 | H | H | H | H |
| 895 | 0 | m-D6 | A-2 | H | H | H | H |
| 896 | 0 | m-D6 | A-3 | H | H | H | H |
| 897 | 0 | m-D6 | A-4 | H | H | H | H |
| 898 | 0 | m-D6 | A-5 | H | H | H | H |
| 899 | 0 | m-D6 | A-6 | H | H | H | H |
| 900 | 0 | m-D6 | A-7 | H | H | H | H |
| 901 | 0 | m-D6 | A-8 | H | H | H | H |
| 902 | 0 | m-D6 | A-9 | H | H | H | H |
| 903 | 0 | m-D6 | A-10 | H | H | H | H |
| 904 | 0 | m-D6 | A-11 | H | H | H | H |
| 905 | 0 | m-D6 | A-12 | H | H | H | H |
| 906 | 0 | m-D6 | A-13 | H | H | H | H |
| 907 | 0 | m-D6 | A-14 | H | H | H | H |
| 908 | 0 | m-D6 | A-15 | H | H | H | H |
| 909 | 0 | m-D6 | A-16 | H | H | H | H |
| 910 | 0 | m-D6 | A-17 | H | H | H | H |
| 911 | 0 | m-D6 | A-18 | H | H | H | H |
| 912 | 0 | m-D6 | A-19 | H | H | H | H |
| 913 | 0 | m-D6 | A-20 | H | H | H | H |
| 914 | 0 | m-D6 | A-21 | H | H | H | H |
| 915 | 0 | m-D6 | A-22 | H | H | H | H |
| 916 | 0 | m-D6 | A-23 | H | H | H | H |
| 917 | 0 | m-D6 | A-24 | H | H | H | H |
| 918 | 0 | m-D6 | A-25 | H | H | H | H |
| 919 | 0 | m-D6 | A-26 | H | H | H | H |
| 920 | 0 | m-D6 | A-27 | H | H | H | H |
| 921 | 0 | m-D6 | A-28 | H | H | H | H |
| 922 | 0 | m-D6 | A-29 | H | H | H | H |
| 923 | 0 | m-D6 | A-30 | H | H | H | H |
| 924 | 0 | m-D6 | A-31 | H | H | H | H |
| 925 | 0 | m-D6 | A-32 | H | H | H | H |
| 926 | 0 | m-D6 | A-33 | H | H | H | H |
| 927 | 0 | m-D6 | A-34 | H | H | H | H |
| 928 | 0 | m-D6 | A-35 | H | H | H | H |
| 929 | 0 | m-D6 | A-36 | H | H | H | H |
| 930 | 0 | m-D6 | A-37 | H | H | H | H |
| 931 | 0 | m-D6 | A-38 | H | H | H | H |
| 932 | 0 | m-D6 | A-39 | H | H | H | H |
| 933 | 0 | m-D6 | A-40 | H | H | H | H |
| 934 | 0 | m-D6 | A-41 | H | H | H | H |
| 935 | 0 | m-D6 | A-42 | H | H | H | H |
| 936 | 0 | m-D6 | A-43 | H | H | H | H |
| 937 | 0 | m-D6 | A-44 | H | H | H | H |
| 938 | 0 | m-D6 | A-45 | H | H | H | H |
| 939 | 0 | m-D6 | A-46 | H | H | H | H |
| 940 | 0 | m-D6 | A-47 | H | H | H | H |
| 941 | 0 | m-D7 | A-1 | H | H | H | H |
| 942 | 0 | m-D7 | A-2 | H | H | H | H |
| 943 | 0 | m-D7 | A-3 | H | H | H | H |
| 944 | 0 | m-D7 | A-4 | H | H | H | H |
| 945 | 0 | m-D7 | A-5 | H | H | H | H |
| 946 | 0 | m-D7 | A-6 | H | H | H | H |
| 947 | 0 | m-D7 | A-7 | H | H | H | H |
| 948 | 0 | m-D7 | A-8 | H | H | H | H |
| 949 | 0 | m-D7 | A-9 | H | H | H | H |
| 950 | 0 | m-D7 | A-10 | H | H | H | H |
| 951 | 0 | m-D7 | A-11 | H | H | H | H |
| 952 | 0 | m-D7 | A-12 | H | H | H | H |
| 953 | 0 | m-D7 | A-13 | H | H | H | H |
| 954 | 0 | m-D7 | A-14 | H | H | H | H |
| 955 | 0 | m-D7 | A-15 | H | H | H | H |
| 956 | 0 | m-D7 | A-16 | H | H | H | H |
| 957 | 0 | m-D7 | A-17 | H | H | H | H |
| 958 | 0 | m-D7 | A-18 | H | H | H | H |
| 959 | 0 | m-D7 | A-19 | H | H | H | H |
| 960 | 0 | m-D7 | A-20 | H | H | H | H |
| 961 | 0 | m-D7 | A-21 | H | H | H | H |
| 962 | 0 | m-D7 | A-22 | H | H | H | H |
| 963 | 0 | m-D7 | A-23 | H | H | H | H |
| 964 | 0 | m-D7 | A-24 | H | H | H | H |
| 965 | 0 | m-D7 | A-25 | H | H | H | H |
| 966 | 0 | m-D7 | A-26 | H | H | H | H |
| 967 | 0 | m-D7 | A-27 | H | H | H | H |
| 968 | 0 | m-D7 | A-28 | H | H | H | H |
| 969 | 0 | m-D7 | A-29 | H | H | H | H |
| 970 | 0 | m-D7 | A-30 | H | H | H | H |
| 971 | 0 | m-D7 | A-31 | H | H | H | H |
| 972 | 0 | m-D7 | A-32 | H | H | H | H |
| 973 | 0 | m-D7 | A-33 | H | H | H | H |
| 974 | 0 | m-D7 | A-34 | H | H | H | H |
| 975 | 0 | m-D7 | A-35 | H | H | H | H |
| 976 | 0 | m-D7 | A-36 | H | H | H | H |
| 977 | 0 | m-D7 | A-37 | H | H | H | H |
| 978 | 0 | m-D7 | A-38 | H | H | H | H |
| 979 | 0 | m-D7 | A-39 | H | H | H | H |
| 980 | 0 | m-D7 | A-40 | H | H | H | H |
| 981 | 0 | m-D7 | A-41 | H | H | H | H |
| 982 | 0 | m-D7 | A-42 | H | H | H | H |
| 983 | 0 | m-D7 | A-43 | H | H | H | H |
| 984 | 0 | m-D7 | A-44 | H | H | H | H |
| 985 | 0 | m-D7 | A-45 | H | H | H | H |
| 986 | 0 | m-D7 | A-46 | H | H | H | H |
| 987 | 0 | m-D7 | A-47 | H | H | H | H |
| 988 | 0 | m-D8 | A-1 | H | H | H | H |
| 989 | 0 | m-D8 | A-2 | H | H | H | H |
| 990 | 0 | m-D8 | A-3 | H | H | H | H |
| 991 | 0 | m-D8 | A-4 | H | H | H | H |
| 992 | 0 | m-D8 | A-5 | H | H | H | H |
| 993 | 0 | m-D8 | A-6 | H | H | H | H |
| 994 | 0 | m-D8 | A-7 | H | H | H | H |
| 995 | 0 | m-D8 | A-8 | H | H | H | H |
| 996 | 0 | m-D8 | A-9 | H | H | H | H |
| 997 | 0 | m-D8 | A-10 | H | H | H | H |
| 998 | 0 | m-D8 | A-11 | H | H | H | H |
| 999 | 0 | m-D8 | A-12 | H | H | H | H |
| 1000 | 0 | m-D8 | A-13 | H | H | H | H |
| 1001 | 0 | m-D8 | A-14 | H | H | H | H |
| 1002 | 0 | m-D8 | A-15 | H | H | H | H |
| 1003 | 0 | m-D8 | A-16 | H | H | H | H |
| 1004 | 0 | m-D8 | A-17 | H | H | H | H |
| 1005 | 0 | m-D8 | A-18 | H | H | H | H |
| 1006 | 0 | m-D8 | A-19 | H | H | H | H |
| 1007 | 0 | m-D8 | A-20 | H | H | H | H |
| 1008 | 0 | m-D8 | A-21 | H | H | H | H |
| 1009 | 0 | m-D8 | A-22 | H | H | H | H |
| 1010 | 0 | m-D8 | A-23 | H | H | H | H |
| 1011 | 0 | m-D8 | A-24 | H | H | H | H |
| 1012 | 0 | m-D8 | A-25 | H | H | H | H |
| 1013 | 0 | m-D8 | A-26 | H | H | H | H |
| 1014 | 0 | m-D8 | A-27 | H | H | H | H |
| 1015 | 0 | m-D8 | A-28 | H | H | H | H |
| 1016 | 0 | m-D8 | A-29 | H | H | H | H |
| 1017 | 0 | m-D8 | A-30 | H | H | H | H |
| 1018 | 0 | m-D8 | A-31 | H | H | H | H |
| 1019 | 0 | m-D8 | A-32 | H | H | H | H |
| 1020 | 0 | m-D8 | A-33 | H | H | H | H |
| 1021 | 0 | m-D8 | A-34 | H | H | H | H |
| 1022 | 0 | m-D8 | A-35 | H | H | H | H |
| 1023 | 0 | m-D8 | A-36 | H | H | H | H |
| 1024 | 0 | m-D8 | A-37 | H | H | H | H |
| 1025 | 0 | m-D8 | A-38 | H | H | H | H |
| 1026 | 0 | m-D8 | A-39 | H | H | H | H |
| 1027 | 0 | m-D8 | A-40 | H | H | H | H |
| 1028 | 0 | m-D8 | A-41 | H | H | H | H |
| 1029 | 0 | m-D8 | A-42 | H | H | H | H |
| 1030 | 0 | m-D8 | A-43 | H | H | H | H |
| 1031 | 0 | m-D8 | A-44 | H | H | H | H |
| 1032 | 0 | m-D8 | A-45 | H | H | H | H |
| 1033 | 0 | m-D8 | A-46 | H | H | H | H |
| 1034 | 0 | m-D8 | A-47 | H | H | H | H |
| 1035 | 0 | m-D9 | A-1 | H | H | H | H |
| 1036 | 0 | m-D9 | A-2 | H | H | H | H |
| 1037 | 0 | m-D9 | A-3 | H | H | H | H |
| 1038 | 0 | m-D9 | A-4 | H | H | H | H |
| 1039 | 0 | m-D9 | A-5 | H | H | H | H |
| 1040 | 0 | m-D9 | A-6 | H | H | H | H |
| 1041 | 0 | m-D9 | A-7 | H | H | H | H |
| 1042 | 0 | m-D9 | A-8 | H | H | H | H |
| 1043 | 0 | m-D9 | A-9 | H | H | H | H |
| 1044 | 0 | m-D9 | A-10 | H | H | H | H |
| 1045 | 0 | m-D9 | A-11 | H | H | H | H |
| 1046 | 0 | m-D9 | A-12 | H | H | H | H |
| 1047 | 0 | m-D9 | A-13 | H | H | H | H |
| 1048 | 0 | m-D9 | A-14 | H | H | H | H |
| 1049 | 0 | m-D9 | A-15 | H | H | H | H |
| 1050 | 0 | m-D9 | A-16 | H | H | H | H |
| 1051 | 0 | m-D9 | A-17 | H | H | H | H |
| 1052 | 0 | m-D9 | A-18 | H | H | H | H |
| 1053 | 0 | m-D9 | A-19 | H | H | H | H |
| 1054 | 0 | m-D9 | A-20 | H | H | H | H |
| 1055 | 0 | m-D9 | A-21 | H | H | H | H |
| 1056 | 0 | m-D9 | A-22 | H | H | H | H |
| 1057 | 0 | m-D9 | A-23 | H | H | H | H |
| 1058 | 0 | m-D9 | A-24 | H | H | H | H |
| 1059 | 0 | m-D9 | A-25 | H | H | H | H |
| 1060 | 0 | m-D9 | A-26 | H | H | H | H |
| 1061 | 0 | m-D9 | A-27 | H | H | H | H |
| 1062 | 0 | m-D9 | A-28 | H | H | H | H |
| 1063 | 0 | m-D9 | A-29 | H | H | H | H |
| 1064 | 0 | m-D9 | A-30 | H | H | H | H |
| 1065 | 0 | m-D9 | A-31 | H | H | H | H |
| 1066 | 0 | m-D9 | A-32 | H | H | H | H |
| 1067 | 0 | m-D9 | A-33 | H | H | H | H |
| 1068 | 0 | m-D9 | A-34 | H | H | H | H |
| 1069 | 0 | m-D9 | A-35 | H | H | H | H |
| 1070 | 0 | m-D9 | A-36 | H | H | H | H |
| 1071 | 0 | m-D9 | A-37 | H | H | H | H |
| 1072 | 0 | m-D9 | A-38 | H | H | H | H |
| 1073 | 0 | m-D9 | A-39 | H | H | H | H |
| 1074 | 0 | m-D9 | A-40 | H | H | H | H |
| 1075 | 0 | m-D9 | A-41 | H | H | H | H |
| 1076 | 0 | m-D9 | A-42 | H | H | H | H |
| 1077 | 0 | m-D9 | A-43 | H | H | H | H |
| 1078 | 0 | m-D9 | A-44 | H | H | H | H |
| 1079 | 0 | m-D9 | A-45 | H | H | H | H |
| 1080 | 0 | m-D9 | A-46 | H | H | H | H |
| 1081 | 0 | m-D9 | A-47 | H | H | H | H |
| 1082 | 0 | m-D10 | A-1 | H | H | H | H |
| 1083 | 0 | m-D10 | A-2 | H | H | H | H |
| 1084 | 0 | m-D10 | A-3 | H | H | H | H |
| 1085 | 0 | m-D10 | A-4 | H | H | H | H |
| 1086 | 0 | m-D10 | A-5 | H | H | H | H |
| 1087 | 0 | m-D10 | A-6 | H | H | H | H |
| 1088 | 0 | m-D10 | A-7 | H | H | H | H |
| 1089 | 0 | m-D10 | A-8 | H | H | H | H |
| 1090 | 0 | m-D10 | A-9 | H | H | H | H |
| 1091 | 0 | m-D10 | A-10 | H | H | H | H |
| 1092 | 0 | m-D10 | A-11 | H | H | H | H |
| 1093 | 0 | m-D10 | A-12 | H | H | H | H |
| 1094 | 0 | m-D10 | A-13 | H | H | H | H |
| 1095 | 0 | m-D10 | A-14 | H | H | H | H |
| 1096 | 0 | m-D10 | A-15 | H | H | H | H |
| 1097 | 0 | m-D10 | A-16 | H | H | H | H |
| 1098 | 0 | m-D10 | A-17 | H | H | H | H |
| 1099 | 0 | m-D10 | A-18 | H | H | H | H |
| 1100 | 0 | m-D10 | A-19 | H | H | H | H |
| 1101 | 0 | m-D10 | A-20 | H | H | H | H |
| 1102 | 0 | m-D10 | A-21 | H | H | H | H |
| 1103 | 0 | m-D10 | A-22 | H | H | H | H |
| 1104 | 0 | m-D10 | A-23 | H | H | H | H |
| 1105 | 0 | m-D10 | A-24 | H | H | H | H |
| 1106 | 0 | m-D10 | A-25 | H | H | H | H |
| 1107 | 0 | m-D10 | A-26 | H | H | H | H |
| 1108 | 0 | m-D10 | A-27 | H | H | H | H |
| 1109 | 0 | m-D10 | A-28 | H | H | H | H |
| 1110 | 0 | m-D10 | A-29 | H | H | H | H |
| 1111 | 0 | m-D10 | A-30 | H | H | H | H |
| 1112 | 0 | m-D10 | A-31 | H | H | H | H |
| 1113 | 0 | m-D10 | A-32 | H | H | H | H |
| 1114 | 0 | m-D10 | A-33 | H | H | H | H |
| 1115 | 0 | m-D10 | A-34 | H | H | H | H |
| 1116 | 0 | m-D10 | A-35 | H | H | H | H |
| 1117 | 0 | m-D10 | A-36 | H | H | H | H |
| 1118 | 0 | m-D10 | A-37 | H | H | H | H |
| 1119 | 0 | m-D10 | A-38 | H | H | H | H |
| 1120 | 0 | m-D10 | A-39 | H | H | H | H |
| 1121 | 0 | m-D10 | A-40 | H | H | H | H |
| 1122 | 0 | m-D10 | A-41 | H | H | H | H |
| 1123 | 0 | m-D10 | A-42 | H | H | H | H |
| 1124 | 0 | m-D10 | A-43 | H | H | H | H |
| 1125 | 0 | m-D10 | A-44 | H | H | H | H |
| 1126 | 0 | m-D10 | A-45 | H | H | H | H |
| 1127 | 0 | m-D10 | A-46 | H | H | H | H |
| 1128 | 0 | m-D10 | A-47 | H | H | H | H |
| 1129 | 0 | m-D11 | A-1 | H | H | H | H |
| 1130 | 0 | m-D11 | A-2 | H | H | H | H |
| 1131 | 0 | m-D11 | A-3 | H | H | H | H |
| 1132 | 0 | m-D11 | A-4 | H | H | H | H |
| 1133 | 0 | m-D11 | A-5 | H | H | H | H |
| 1134 | 0 | m-D11 | A-6 | H | H | H | H |
| 1135 | 0 | m-D11 | A-7 | H | H | H | H |
| 1136 | 0 | m-D11 | A-8 | H | H | H | H |
| 1137 | 0 | m-D11 | A-9 | H | H | H | H |
| 1138 | 0 | m-D11 | A-10 | H | H | H | H |
| 1139 | 0 | m-D11 | A-11 | H | H | H | H |
| 1140 | 0 | m-D11 | A-12 | H | H | H | H |
| 1141 | 0 | m-D11 | A-13 | H | H | H | H |
| 1142 | 0 | m-D11 | A-14 | H | H | H | H |
| 1143 | 0 | m-D11 | A-15 | H | H | H | H |
| 1144 | 0 | m-D11 | A-16 | H | H | H | H |
| 1145 | 0 | m-D11 | A-17 | H | H | H | H |
| 1146 | 0 | m-D11 | A-18 | H | H | H | H |
| 1147 | 0 | m-D11 | A-19 | H | H | H | H |
| 1148 | 0 | m-D11 | A-20 | H | H | H | H |
| 1149 | 0 | m-D11 | A-21 | H | H | H | H |
| 1150 | 0 | m-D11 | A-22 | H | H | H | H |
| 1151 | 0 | m-D11 | A-23 | H | H | H | H |
| 1152 | 0 | m-D11 | A-24 | H | H | H | H |
| 1153 | 0 | m-D11 | A-25 | H | H | H | H |
| 1154 | 0 | m-D11 | A-26 | H | H | H | H |
| 1155 | 0 | m-D11 | A-27 | H | H | H | H |
| 1156 | 0 | m-D11 | A-28 | H | H | H | H |
| 1157 | 0 | m-D11 | A-29 | H | H | H | H |
| 1158 | 0 | m-D11 | A-30 | H | H | H | H |
| 1159 | 0 | m-D11 | A-31 | H | H | H | H |
| 1160 | 0 | m-D11 | A-32 | H | H | H | H |
| 1161 | 0 | m-D11 | A-33 | H | H | H | H |
| 1162 | 0 | m-D11 | A-34 | H | H | H | H |
| 1163 | 0 | m-D11 | A-35 | H | H | H | H |
| 1164 | 0 | m-D11 | A-36 | H | H | H | H |
| 1165 | 0 | m-D11 | A-37 | H | H | H | H |
| 1166 | 0 | m-D11 | A-38 | H | H | H | H |
| 1167 | 0 | m-D11 | A-39 | H | H | H | H |
| 1168 | 0 | m-D11 | A-40 | H | H | H | H |
| 1169 | 0 | m-D11 | A-41 | H | H | H | H |
| 1170 | 0 | m-D11 | A-42 | H | H | H | H |
| 1171 | 0 | m-D11 | A-43 | H | H | H | H |
| 1172 | 0 | m-D11 | A-44 | H | H | H | H |
| 1173 | 0 | m-D11 | A-45 | H | H | H | H |
| 1174 | 0 | m-D11 | A-46 | H | H | H | H |
| 1175 | 0 | m-D11 | A-47 | H | H | H | H |
| 1176 | 0 | m-D12 | A-1 | H | H | H | H |
| 1177 | 0 | m-D12 | A-2 | H | H | H | H |
| 1178 | 0 | m-D12 | A-3 | H | H | H | H |
| 1179 | 0 | m-D12 | A-4 | H | H | H | H |
| 1180 | 0 | m-D12 | A-5 | H | H | H | H |
| 1181 | 0 | m-D12 | A-6 | H | H | H | H |
| 1182 | 0 | m-D12 | A-7 | H | H | H | H |
| 1183 | 0 | m-D12 | A-8 | H | H | H | H |
| 1184 | 0 | m-D12 | A-9 | H | H | H | H |
| 1185 | 0 | m-D12 | A-10 | H | H | H | H |
| 1186 | 0 | m-D12 | A-11 | H | H | H | H |
| 1187 | 0 | m-D12 | A-12 | H | H | H | H |
| 1188 | 0 | m-D12 | A-13 | H | H | H | H |
| 1189 | 0 | m-D12 | A-14 | H | H | H | H |
| 1190 | 0 | m-D12 | A-15 | H | H | H | H |
| 1191 | 0 | m-D12 | A-16 | H | H | H | H |
| 1192 | 0 | m-D12 | A-17 | H | H | H | H |
| 1193 | 0 | m-D12 | A-18 | H | H | H | H |
| 1194 | 0 | m-D12 | A-19 | H | H | H | H |
| 1195 | 0 | m-D12 | A-20 | H | H | H | H |
| 1196 | 0 | m-D12 | A-21 | H | H | H | H |
| 1197 | 0 | m-D12 | A-22 | H | H | H | H |
| 1198 | 0 | m-D12 | A-23 | H | H | H | H |
| 1199 | 0 | m-D12 | A-24 | H | H | H | H |
| 1200 | 0 | m-D12 | A-25 | H | H | H | H |
| 1201 | 0 | m-D12 | A-26 | H | H | H | H |
| 1202 | 0 | m-D12 | A-27 | H | H | H | H |
| 1203 | 0 | m-D12 | A-28 | H | H | H | H |
| 1204 | 0 | m-D12 | A-29 | H | H | H | H |
| 1205 | 0 | m-D12 | A-30 | H | H | H | H |
| 1206 | 0 | m-D12 | A-31 | H | H | H | H |
| 1207 | 0 | m-D12 | A-32 | H | H | H | H |
| 1208 | 0 | m-D12 | A-33 | H | H | H | H |
| 1209 | 0 | m-D12 | A-34 | H | H | H | H |
| 1210 | 0 | m-D12 | A-35 | H | H | H | H |
| 1211 | 0 | m-D12 | A-36 | H | H | H | H |
| 1212 | 0 | m-D12 | A-37 | H | H | H | H |
| 1213 | 0 | m-D12 | A-38 | H | H | H | H |
| 1214 | 0 | m-D12 | A-39 | H | H | H | H |
| 1215 | 0 | m-D12 | A-40 | H | H | H | H |
| 1216 | 0 | m-D12 | A-41 | H | H | H | H |
| 1217 | 0 | m-D12 | A-42 | H | H | H | H |
| 1218 | 0 | m-D12 | A-43 | H | H | H | H |
| 1219 | 0 | m-D12 | A-44 | H | H | H | H |
| 1220 | 0 | m-D12 | A-45 | H | H | H | H |
| 1221 | 0 | m-D12 | A-46 | H | H | H | H |
| 1222 | 0 | m-D12 | A-47 | H | H | H | H |
| 1223 | 0 | m-D13 | A-1 | H | H | H | H |
| 1224 | 0 | m-D13 | A-2 | H | H | H | H |
| 1225 | 0 | m-D13 | A-3 | H | H | H | H |
| 1226 | 0 | m-D13 | A-4 | H | H | H | H |
| 1227 | 0 | m-D13 | A-5 | H | H | H | H |
| 1228 | 0 | m-D13 | A-6 | H | H | H | H |
| 1229 | 0 | m-D13 | A-7 | H | H | H | H |
| 1230 | 0 | m-D13 | A-8 | H | H | H | H |
| 1231 | 0 | m-D13 | A-9 | H | H | H | H |
| 1232 | 0 | m-D13 | A-10 | H | H | H | H |
| 1233 | 0 | m-D13 | A-11 | H | H | H | H |
| 1234 | 0 | m-D13 | A-12 | H | H | H | H |
| 1235 | 0 | m-D13 | A-13 | H | H | H | H |
| 1236 | 0 | m-D13 | A-14 | H | H | H | H |
| 1237 | 0 | m-D13 | A-15 | H | H | H | H |
| 1238 | 0 | m-D13 | A-16 | H | H | H | H |
| 1239 | 0 | m-D13 | A-17 | H | H | H | H |
| 1240 | 0 | m-D13 | A-18 | H | H | H | H |
| 1241 | 0 | m-D13 | A-19 | H | H | H | H |
| 1242 | 0 | m-D13 | A-20 | H | H | H | H |
| 1243 | 0 | m-D13 | A-21 | H | H | H | H |
| 1244 | 0 | m-D13 | A-22 | H | H | H | H |
| 1245 | 0 | m-D13 | A-23 | H | H | H | H |
| 1246 | 0 | m-D13 | A-24 | H | H | H | H |
| 1247 | 0 | m-D13 | A-25 | H | H | H | H |
| 1248 | 0 | m-D13 | A-26 | H | H | H | H |
| 1249 | 0 | m-D13 | A-27 | H | H | H | H |
| 1250 | 0 | m-D13 | A-28 | H | H | H | H |
| 1251 | 0 | m-D13 | A-29 | H | H | H | H |
| 1252 | 0 | m-D13 | A-30 | H | H | H | H |
| 1253 | 0 | m-D13 | A-31 | H | H | H | H |
| 1254 | 0 | m-D13 | A-32 | H | H | H | H |
| 1255 | 0 | m-D13 | A-33 | H | H | H | H |
| 1256 | 0 | m-D13 | A-34 | H | H | H | H |
| 1257 | 0 | m-D13 | A-35 | H | H | H | H |
| 1258 | 0 | m-D13 | A-36 | H | H | H | H |
| 1259 | 0 | m-D13 | A-37 | H | H | H | H |
| 1260 | 0 | m-D13 | A-38 | H | H | H | H |
| 1261 | 0 | m-D13 | A-39 | H | H | H | H |
| 1262 | 0 | m-D13 | A-40 | H | H | H | H |
| 1263 | 0 | m-D13 | A-41 | H | H | H | H |
| 1264 | 0 | m-D13 | A-42 | H | H | H | H |
| 1265 | 0 | m-D13 | A-43 | H | H | H | H |
| 1266 | 0 | m-D13 | A-44 | H | H | H | H |
| 1267 | 0 | m-D13 | A-45 | H | H | H | H |
| 1268 | 0 | m-D13 | A-46 | H | H | H | H |
| 1269 | 0 | m-D13 | A-47 | H | H | H | H |
| 1270 | 0 | m-D14 | A-1 | H | H | H | H |
| 1271 | 0 | m-D14 | A-2 | H | H | H | H |
| 1272 | 0 | m-D14 | A-3 | H | H | H | H |
| 1273 | 0 | m-D14 | A-4 | H | H | H | H |
| 1274 | 0 | m-D14 | A-5 | H | H | H | H |
| 1275 | 0 | m-D14 | A-6 | H | H | H | H |
| 1276 | 0 | m-D14 | A-7 | H | H | H | H |
| 1277 | 0 | m-D14 | A-8 | H | H | H | H |
| 1278 | 0 | m-D14 | A-9 | H | H | H | H |
| 1279 | 0 | m-D14 | A-10 | H | H | H | H |
| 1280 | 0 | m-D14 | A-11 | H | H | H | H |
| 1281 | 0 | m-D14 | A-12 | H | H | H | H |
| 1282 | 0 | m-D14 | A-13 | H | H | H | H |
| 1283 | 0 | m-D14 | A-14 | H | H | H | H |
| 1284 | 0 | m-D14 | A-15 | H | H | H | H |
| 1285 | 0 | m-D14 | A-16 | H | H | H | H |
| 1286 | 0 | m-D14 | A-17 | H | H | H | H |
| 1287 | 0 | m-D14 | A-18 | H | H | H | H |
| 1288 | 0 | m-D14 | A-19 | H | H | H | H |
| 1289 | 0 | m-D14 | A-20 | H | H | H | H |
| 1290 | 0 | m-D14 | A-21 | H | H | H | H |
| 1291 | 0 | m-D14 | A-22 | H | H | H | H |
| 1292 | 0 | m-D14 | A-23 | H | H | H | H |
| 1293 | 0 | m-D14 | A-24 | H | H | H | H |
| 1294 | 0 | m-D14 | A-25 | H | H | H | H |
| 1295 | 0 | m-D14 | A-26 | H | H | H | H |
| 1296 | 0 | m-D14 | A-27 | H | H | H | H |
| 1297 | 0 | m-D14 | A-28 | H | H | H | H |
| 1298 | 0 | m-D14 | A-29 | H | H | H | H |
| 1299 | 0 | m-D14 | A-30 | H | H | H | H |
| 1300 | 0 | m-D14 | A-31 | H | H | H | H |
| 1301 | 0 | m-D14 | A-32 | H | H | H | H |
| 1302 | 0 | m-D14 | A-33 | H | H | H | H |
| 1303 | 0 | m-D14 | A-34 | H | H | H | H |
| 1304 | 0 | m-D14 | A-35 | H | H | H | H |
| 1305 | 0 | m-D14 | A-36 | H | H | H | H |
| 1306 | 0 | m-D14 | A-37 | H | H | H | H |
| 1307 | 0 | m-D14 | A-38 | H | H | H | H |
| 1308 | 0 | m-D14 | A-39 | H | H | H | H |
| 1309 | 0 | m-D14 | A-40 | H | H | H | H |
| 1310 | 0 | m-D14 | A-41 | H | H | H | H |
| 1311 | 0 | m-D14 | A-42 | H | H | H | H |
| 1312 | 0 | m-D14 | A-43 | H | H | H | H |
| 1313 | 0 | m-D14 | A-44 | H | H | H | H |
| 1314 | 0 | m-D14 | A-45 | H | H | H | H |
| 1315 | 0 | m-D14 | A-46 | H | H | H | H |
| 1316 | 0 | m-D14 | A-47 | H | H | H | H |
| 1317 | 0 | m-D1 | H | A-1 | H | H | H |
| 1318 | 0 | m-D1 | H | A-2 | H | H | H |
| 1319 | 0 | m-D1 | H | A-3 | H | H | H |
| 1320 | 0 | m-D1 | H | A-4 | H | H | H |
| 1321 | 0 | m-D1 | H | A-5 | H | H | H |
| 1322 | 0 | m-D1 | H | A-6 | H | H | H |
| 1323 | 0 | m-D1 | H | A-7 | H | H | H |
| 1324 | 0 | m-D1 | H | A-8 | H | H | H |
| 1325 | 0 | m-D1 | H | A-9 | H | H | H |
| 1326 | 0 | m-D1 | H | A-10 | H | H | H |
| 1327 | 0 | m-D1 | H | A-11 | H | H | H |
| 1328 | 0 | m-D1 | H | A-12 | H | H | H |
| 1329 | 0 | m-D1 | H | A-13 | H | H | H |
| 1330 | 0 | m-D1 | H | A-14 | H | H | H |
| 1331 | 0 | m-D1 | H | A-15 | H | H | H |
| 1332 | 0 | m-D1 | H | A-16 | H | H | H |
| 1333 | 0 | m-D1 | H | A-17 | H | H | H |
| 1334 | 0 | m-D1 | H | A-18 | H | H | H |
| 1335 | 0 | m-D1 | H | A-19 | H | H | H |
| 1336 | 0 | m-D1 | H | A-20 | H | H | H |
| 1337 | 0 | m-D1 | H | A-21 | H | H | H |
| 1338 | 0 | m-D1 | H | A-22 | H | H | H |
| 1339 | 0 | m-D1 | H | A-23 | H | H | H |
| 1340 | 0 | m-D1 | H | A-24 | H | H | H |
| 1341 | 0 | m-D1 | H | A-25 | H | H | H |
| 1342 | 0 | m-D1 | H | A-26 | H | H | H |
| 1343 | 0 | m-D1 | H | A-27 | H | H | H |
| 1344 | 0 | m-D1 | H | A-28 | H | H | H |
| 1345 | 0 | m-D1 | H | A-29 | H | H | H |
| 1346 | 0 | m-D1 | H | A-30 | H | H | H |
| 1347 | 0 | m-D1 | H | A-31 | H | H | H |
| 1348 | 0 | m-D1 | H | A-32 | H | H | H |
| 1349 | 0 | m-D1 | H | A-33 | H | H | H |
| 1350 | 0 | m-D1 | H | A-34 | H | H | H |
| 1351 | 0 | m-D1 | H | A-35 | H | H | H |
| 1352 | 0 | m-D1 | H | A-36 | H | H | H |
| 1353 | 0 | m-D1 | H | A-37 | H | H | H |
| 1354 | 0 | m-D1 | H | A-38 | H | H | H |
| 1355 | 0 | m-D1 | H | A-39 | H | H | H |
| 1356 | 0 | m-D1 | H | A-40 | H | H | H |
| 1357 | 0 | m-D1 | H | A-41 | H | H | H |
| 1358 | 0 | m-D1 | H | A-42 | H | H | H |
| 1359 | 0 | m-D1 | H | A-43 | H | H | H |
| 1360 | 0 | m-D1 | H | A-44 | H | H | H |
| 1361 | 0 | m-D1 | H | A-45 | H | H | H |
| 1362 | 0 | m-D1 | H | A-46 | H | H | H |
| 1363 | 0 | m-D1 | H | A-47 | H | H | H |
| 1364 | 0 | m-D2 | H | A-1 | H | H | H |
| 1365 | 0 | m-D2 | H | A-2 | H | H | H |
| 1366 | 0 | m-D2 | H | A-3 | H | H | H |
| 1367 | 0 | m-D2 | H | A-4 | H | H | H |
| 1368 | 0 | m-D2 | H | A-5 | H | H | H |
| 1369 | 0 | m-D2 | H | A-6 | H | H | H |
| 1370 | 0 | m-D2 | H | A-7 | H | H | H |
| 1371 | 0 | m-D2 | H | A-8 | H | H | H |
| 1372 | 0 | m-D2 | H | A-9 | H | H | H |
| 1373 | 0 | m-D2 | H | A-10 | H | H | H |
| 1374 | 0 | m-D2 | H | A-11 | H | H | H |
| 1375 | 0 | m-D2 | H | A-12 | H | H | H |
| 1376 | 0 | m-D2 | H | A-13 | H | H | H |
| 1377 | 0 | m-D2 | H | A-14 | H | H | H |
| 1378 | 0 | m-D2 | H | A-15 | H | H | H |
| 1379 | 0 | m-D2 | H | A-16 | H | H | H |
| 1380 | 0 | m-D2 | H | A-17 | H | H | H |
| 1381 | 0 | m-D2 | H | A-18 | H | H | H |
| 1382 | 0 | m-D2 | H | A-19 | H | H | H |
| 1383 | 0 | m-D2 | H | A-20 | H | H | H |
| 1384 | 0 | m-D2 | H | A-21 | H | H | H |
| 1385 | 0 | m-D2 | H | A-22 | H | H | H |
| 1386 | 0 | m-D2 | H | A-23 | H | H | H |
| 1387 | 0 | m-D2 | H | A-24 | H | H | H |
| 1388 | 0 | m-D2 | H | A-25 | H | H | H |
| 1389 | 0 | m-D2 | H | A-26 | H | H | H |
| 1390 | 0 | m-D2 | H | A-27 | H | H | H |
| 1391 | 0 | m-D2 | H | A-28 | H | H | H |
| 1392 | 0 | m-D2 | H | A-29 | H | H | H |
| 1393 | 0 | m-D2 | H | A-30 | H | H | H |
| 1394 | 0 | m-D2 | H | A-31 | H | H | H |
| 1395 | 0 | m-D2 | H | A-32 | H | H | H |
| 1396 | 0 | m-D2 | H | A-33 | H | H | H |
| 1397 | 0 | m-D2 | H | A-34 | H | H | H |
| 1398 | 0 | m-D2 | H | A-35 | H | H | H |
| 1399 | 0 | m-D2 | H | A-36 | H | H | H |
| 1400 | 0 | m-D2 | H | A-37 | H | H | H |
| 1401 | 0 | m-D2 | H | A-38 | H | H | H |
| 1402 | 0 | m-D2 | H | A-39 | H | H | H |
| 1403 | 0 | m-D2 | H | A-40 | H | H | H |
| 1404 | 0 | m-D2 | H | A-41 | H | H | H |
| 1405 | 0 | m-D2 | H | A-42 | H | H | H |
| 1406 | 0 | m-D2 | H | A-43 | H | H | H |
| 1407 | 0 | m-D2 | H | A-44 | H | H | H |
| 1408 | 0 | m-D2 | H | A-45 | H | H | H |
| 1409 | 0 | m-D2 | H | A-46 | H | H | H |
| 1410 | 0 | m-D2 | H | A-47 | H | H | H |
| 1411 | 0 | m-D3 | H | A-1 | H | H | H |
| 1412 | 0 | m-D3 | H | A-2 | H | H | H |
| 1413 | 0 | m-D3 | H | A-3 | H | H | H |
| 1414 | 0 | m-D3 | H | A-4 | H | H | H |
| 1415 | 0 | m-D3 | H | A-5 | H | H | H |
| 1416 | 0 | m-D3 | H | A-6 | H | H | H |
| 1417 | 0 | m-D3 | H | A-7 | H | H | H |
| 1418 | 0 | m-D3 | H | A-8 | H | H | H |
| 1419 | 0 | m-D3 | H | A-9 | H | H | H |
| 1420 | 0 | m-D3 | H | A-10 | H | H | H |
| 1421 | 0 | m-D3 | H | A-11 | H | H | H |
| 1422 | 0 | m-D3 | H | A-12 | H | H | H |
| 1423 | 0 | m-D3 | H | A-13 | H | H | H |
| 1424 | 0 | m-D3 | H | A-14 | H | H | H |
| 1425 | 0 | m-D3 | H | A-15 | H | H | H |
| 1426 | 0 | m-D3 | H | A-16 | H | H | H |
| 1427 | 0 | m-D3 | H | A-17 | H | H | H |
| 1428 | 0 | m-D3 | H | A-18 | H | H | H |
| 1429 | 0 | m-D3 | H | A-19 | H | H | H |
| 1430 | 0 | m-D3 | H | A-20 | H | H | H |
| 1431 | 0 | m-D3 | H | A-21 | H | H | H |
| 1432 | 0 | m-D3 | H | A-22 | H | H | H |
| 1433 | 0 | m-D3 | H | A-23 | H | H | H |
| 1434 | 0 | m-D3 | H | A-24 | H | H | H |
| 1435 | 0 | m-D3 | H | A-25 | H | H | H |
| 1436 | 0 | m-D3 | H | A-26 | H | H | H |
| 1437 | 0 | m-D3 | H | A-27 | H | H | H |
| 1438 | 0 | m-D3 | H | A-28 | H | H | H |
| 1439 | 0 | m-D3 | H | A-29 | H | H | H |
| 1440 | 0 | m-D3 | H | A-30 | H | H | H |
| 1441 | 0 | m-D3 | H | A-31 | H | H | H |
| 1442 | 0 | m-D3 | H | A-32 | H | H | H |
| 1443 | 0 | m-D3 | H | A-33 | H | H | H |
| 1444 | 0 | m-D3 | H | A-34 | H | H | H |
| 1445 | 0 | m-D3 | H | A-35 | H | H | H |
| 1446 | 0 | m-D3 | H | A-36 | H | H | H |
| 1447 | 0 | m-D3 | H | A-37 | H | H | H |
| 1448 | 0 | m-D3 | H | A-38 | H | H | H |
| 1449 | 0 | m-D3 | H | A-39 | H | H | H |
| 1450 | 0 | m-D3 | H | A-40 | H | H | H |
| 1451 | 0 | m-D3 | H | A-41 | H | H | H |
| 1452 | 0 | m-D3 | H | A-42 | H | H | H |
| 1453 | 0 | m-D3 | H | A-43 | H | H | H |
| 1454 | 0 | m-D3 | H | A-44 | H | H | H |
| 1455 | 0 | m-D3 | H | A-45 | H | H | H |
| 1456 | 0 | m-D3 | H | A-46 | H | H | H |
| 1457 | 0 | m-D3 | H | A-47 | H | H | H |
| 1458 | 0 | m-D4 | H | A-1 | H | H | H |
| 1459 | 0 | m-D4 | H | A-2 | H | H | H |
| 1460 | 0 | m-D4 | H | A-3 | H | H | H |
| 1461 | 0 | m-D4 | H | A-4 | H | H | H |
| 1462 | 0 | m-D4 | H | A-5 | H | H | H |
| 1463 | 0 | m-D4 | H | A-6 | H | H | H |
| 1464 | 0 | m-D4 | H | A-7 | H | H | H |
| 1465 | 0 | m-D4 | H | A-8 | H | H | H |
| 1466 | 0 | m-D4 | H | A-9 | H | H | H |
| 1467 | 0 | m-D4 | H | A-10 | H | H | H |
| 1468 | 0 | m-D4 | H | A-11 | H | H | H |
| 1469 | 0 | m-D4 | H | A-12 | H | H | H |
| 1470 | 0 | m-D4 | H | A-13 | H | H | H |
| 1471 | 0 | m-D4 | H | A-14 | H | H | H |
| 1472 | 0 | m-D4 | H | A-15 | H | H | H |
| 1473 | 0 | m-D4 | H | A-16 | H | H | H |
| 1474 | 0 | m-D4 | H | A-17 | H | H | H |
| 1475 | 0 | m-D4 | H | A-18 | H | H | H |
| 1476 | 0 | m-D4 | H | A-19 | H | H | H |
| 1477 | 0 | m-D4 | H | A-20 | H | H | H |
| 1478 | 0 | m-D4 | H | A-21 | H | H | H |
| 1479 | 0 | m-D4 | H | A-22 | H | H | H |
| 1480 | 0 | m-D4 | H | A-23 | H | H | H |
| 1481 | 0 | m-D4 | H | A-24 | H | H | H |
| 1482 | 0 | m-D4 | H | A-25 | H | H | H |
| 1483 | 0 | m-D4 | H | A-26 | H | H | H |
| 1484 | 0 | m-D4 | H | A-27 | H | H | H |
| 1485 | 0 | m-D4 | H | A-28 | H | H | H |
| 1486 | 0 | m-D4 | H | A-29 | H | H | H |
| 1487 | 0 | m-D4 | H | A-30 | H | H | H |
| 1488 | 0 | m-D4 | H | A-31 | H | H | H |
| 1489 | 0 | m-D4 | H | A-32 | H | H | H |
| 1490 | 0 | m-D4 | H | A-33 | H | H | H |
| 1491 | 0 | m-D4 | H | A-34 | H | H | H |
| 1492 | 0 | m-D4 | H | A-35 | H | H | H |
| 1493 | 0 | m-D4 | H | A-36 | H | H | H |
| 1494 | 0 | m-D4 | H | A-37 | H | H | H |
| 1495 | 0 | m-D4 | H | A-38 | H | H | H |
| 1496 | 0 | m-D4 | H | A-39 | H | H | H |
| 1497 | 0 | m-D4 | H | A-40 | H | H | H |
| 1498 | 0 | m-D4 | H | A-41 | H | H | H |
| 1499 | 0 | m-D4 | H | A-42 | H | H | H |
| 1500 | 0 | m-D4 | H | A-43 | H | H | H |
| 1501 | 0 | m-D4 | H | A-44 | H | H | H |
| 1502 | 0 | m-D4 | H | A-45 | H | H | H |
| 1503 | 0 | m-D4 | H | A-46 | H | H | H |
| 1504 | 0 | m-D4 | H | A-47 | H | H | H |
| 1505 | 0 | m-D5 | H | A-1 | H | H | H |
| 1506 | 0 | m-D5 | H | A-2 | H | H | H |
| 1507 | 0 | m-D5 | H | A-3 | H | H | H |
| 1508 | 0 | m-D5 | H | A-4 | H | H | H |
| 1509 | 0 | m-D5 | H | A-5 | H | H | H |
| 1510 | 0 | m-D5 | H | A-6 | H | H | H |
| 1511 | 0 | m-D5 | H | A-7 | H | H | H |
| 1512 | 0 | m-D5 | H | A-8 | H | H | H |
| 1513 | 0 | m-D5 | H | A-9 | H | H | H |
| 1514 | 0 | m-D5 | H | A-10 | H | H | H |
| 1515 | 0 | m-D5 | H | A-11 | H | H | H |
| 1516 | 0 | m-D5 | H | A-12 | H | H | H |
| 1517 | 0 | m-D5 | H | A-13 | H | H | H |
| 1518 | 0 | m-D5 | H | A-14 | H | H | H |
| 1519 | 0 | m-D5 | H | A-15 | H | H | H |
| 1520 | 0 | m-D5 | H | A-16 | H | H | H |
| 1521 | 0 | m-D5 | H | A-17 | H | H | H |
| 1522 | 0 | m-D5 | H | A-18 | H | H | H |
| 1523 | 0 | m-D5 | H | A-19 | H | H | H |
| 1524 | 0 | m-D5 | H | A-20 | H | H | H |
| 1525 | 0 | m-D5 | H | A-21 | H | H | H |
| 1526 | 0 | m-D5 | H | A-22 | H | H | H |
| 1527 | 0 | m-D5 | H | A-23 | H | H | H |
| 1528 | 0 | m-D5 | H | A-24 | H | H | H |
| 1529 | 0 | m-D5 | H | A-25 | H | H | H |
| 1530 | 0 | m-D5 | H | A-26 | H | H | H |
| 1531 | 0 | m-D5 | H | A-27 | H | H | H |
| 1532 | 0 | m-D5 | H | A-28 | H | H | H |
| 1533 | 0 | m-D5 | H | A-29 | H | H | H |
| 1534 | 0 | m-D5 | H | A-30 | H | H | H |
| 1535 | 0 | m-D5 | H | A-31 | H | H | H |
| 1536 | 0 | m-D5 | H | A-32 | H | H | H |
| 1537 | 0 | m-D5 | H | A-33 | H | H | H |
| 1538 | 0 | m-D5 | H | A-34 | H | H | H |
| 1539 | 0 | m-D5 | H | A-35 | H | H | H |
| 1540 | 0 | m-D5 | H | A-36 | H | H | H |
| 1541 | 0 | m-D5 | H | A-37 | H | H | H |
| 1542 | 0 | m-D5 | H | A-38 | H | H | H |
| 1543 | 0 | m-D5 | H | A-39 | H | H | H |
| 1544 | 0 | m-D5 | H | A-40 | H | H | H |
| 1545 | 0 | m-D5 | H | A-41 | H | H | H |
| 1546 | 0 | m-D5 | H | A-42 | H | H | H |
| 1547 | 0 | m-D5 | H | A-43 | H | H | H |
| 1548 | 0 | m-D5 | H | A-44 | H | H | H |
| 1549 | 0 | m-D5 | H | A-45 | H | H | H |
| 1550 | 0 | m-D5 | H | A-46 | H | H | H |
| 1551 | 0 | m-D5 | H | A-47 | H | H | H |
| 1552 | 0 | m-D6 | H | A-1 | H | H | H |
| 1553 | 0 | m-D6 | H | A-2 | H | H | H |
| 1554 | 0 | m-D6 | H | A-3 | H | H | H |
| 1555 | 0 | m-D6 | H | A-4 | H | H | H |
| 1556 | 0 | m-D6 | H | A-5 | H | H | H |
| 1557 | 0 | m-D6 | H | A-6 | H | H | H |
| 1558 | 0 | m-D6 | H | A-7 | H | H | H |
| 1559 | 0 | m-D6 | H | A-8 | H | H | H |
| 1560 | 0 | m-D6 | H | A-9 | H | H | H |
| 1561 | 0 | m-D6 | H | A-10 | H | H | H |
| 1562 | 0 | m-D6 | H | A-11 | H | H | H |
| 1563 | 0 | m-D6 | H | A-12 | H | H | H |
| 1564 | 0 | m-D6 | H | A-13 | H | H | H |
| 1565 | 0 | m-D6 | H | A-14 | H | H | H |
| 1566 | 0 | m-D6 | H | A-15 | H | H | H |
| 1567 | 0 | m-D6 | H | A-16 | H | H | H |
| 1568 | 0 | m-D6 | H | A-17 | H | H | H |
| 1569 | 0 | m-D6 | H | A-18 | H | H | H |
| 1570 | 0 | m-D6 | H | A-19 | H | H | H |
| 1571 | 0 | m-D6 | H | A-20 | H | H | H |
| 1572 | 0 | m-D6 | H | A-21 | H | H | H |
| 1573 | 0 | m-D6 | H | A-22 | H | H | H |
| 1574 | 0 | m-D6 | H | A-23 | H | H | H |
| 1575 | 0 | m-D6 | H | A-24 | H | H | H |
| 1576 | 0 | m-D6 | H | A-25 | H | H | H |
| 1577 | 0 | m-D6 | H | A-26 | H | H | H |
| 1578 | 0 | m-D6 | H | A-27 | H | H | H |
| 1579 | 0 | m-D6 | H | A-28 | H | H | H |
| 1580 | 0 | m-D6 | H | A-29 | H | H | H |
| 1581 | 0 | m-D6 | H | A-30 | H | H | H |
| 1582 | 0 | m-D6 | H | A-31 | H | H | H |
| 1583 | 0 | m-D6 | H | A-32 | H | H | H |
| 1584 | 0 | m-D6 | H | A-33 | H | H | H |
| 1585 | 0 | m-D6 | H | A-34 | H | H | H |
| 1586 | 0 | m-D6 | H | A-35 | H | H | H |
| 1587 | 0 | m-D6 | H | A-36 | H | H | H |
| 1588 | 0 | m-D6 | H | A-37 | H | H | H |
| 1589 | 0 | m-D6 | H | A-38 | H | H | H |
| 1590 | 0 | m-D6 | H | A-39 | H | H | H |
| 1591 | 0 | m-D6 | H | A-40 | H | H | H |
| 1592 | 0 | m-D6 | H | A-41 | H | H | H |
| 1593 | 0 | m-D6 | H | A-42 | H | H | H |
| 1594 | 0 | m-D6 | H | A-43 | H | H | H |
| 1595 | 0 | m-D6 | H | A-44 | H | H | H |
| 1596 | 0 | m-D6 | H | A-45 | H | H | H |
| 1597 | 0 | m-D6 | H | A-46 | H | H | H |
| 1598 | 0 | m-D6 | H | A-47 | H | H | H |
| 1599 | 0 | m-D7 | H | A-1 | H | H | H |
| 1600 | 0 | m-D7 | H | A-2 | H | H | H |
| 1601 | 0 | m-D7 | H | A-3 | H | H | H |
| 1602 | 0 | m-D7 | H | A-4 | H | H | H |
| 1603 | 0 | m-D7 | H | A-5 | H | H | H |
| 1604 | 0 | m-D7 | H | A-6 | H | H | H |
| 1605 | 0 | m-D7 | H | A-7 | H | H | H |
| 1606 | 0 | m-D7 | H | A-8 | H | H | H |
| 1607 | 0 | m-D7 | H | A-9 | H | H | H |
| 1608 | 0 | m-D7 | H | A-10 | H | H | H |
| 1609 | 0 | m-D7 | H | A-11 | H | H | H |
| 1610 | 0 | m-D7 | H | A-12 | H | H | H |
| 1611 | 0 | m-D7 | H | A-13 | H | H | H |
| 1612 | 0 | m-D7 | H | A-14 | H | H | H |
| 1613 | 0 | m-D7 | H | A-15 | H | H | H |
| 1614 | 0 | m-D7 | H | A-16 | H | H | H |
| 1615 | 0 | m-D7 | H | A-17 | H | H | H |
| 1616 | 0 | m-D7 | H | A-18 | H | H | H |
| 1617 | 0 | m-D7 | H | A-19 | H | H | H |
| 1618 | 0 | m-D7 | H | A-20 | H | H | H |
| 1619 | 0 | m-D7 | H | A-21 | H | H | H |
| 1620 | 0 | m-D7 | H | A-22 | H | H | H |
| 1621 | 0 | m-D7 | H | A-23 | H | H | H |
| 1622 | 0 | m-D7 | H | A-24 | H | H | H |
| 1623 | 0 | m-D7 | H | A-25 | H | H | H |
| 1624 | 0 | m-D7 | H | A-26 | H | H | H |
| 1625 | 0 | m-D7 | H | A-27 | H | H | H |
| 1626 | 0 | m-D7 | H | A-28 | H | H | H |
| 1627 | 0 | m-D7 | H | A-29 | H | H | H |
| 1628 | 0 | m-D7 | H | A-30 | H | H | H |
| 1629 | 0 | m-D7 | H | A-31 | H | H | H |
| 1630 | 0 | m-D7 | H | A-32 | H | H | H |
| 1631 | 0 | m-D7 | H | A-33 | H | H | H |
| 1632 | 0 | m-D7 | H | A-34 | H | H | H |
| 1633 | 0 | m-D7 | H | A-35 | H | H | H |
| 1634 | 0 | m-D7 | H | A-36 | H | H | H |
| 1635 | 0 | m-D7 | H | A-37 | H | H | H |
| 1636 | 0 | m-D7 | H | A-38 | H | H | H |
| 1637 | 0 | m-D7 | H | A-39 | H | H | H |
| 1638 | 0 | m-D7 | H | A-40 | H | H | H |
| 1639 | 0 | m-D7 | H | A-41 | H | H | H |
| 1640 | 0 | m-D7 | H | A-42 | H | H | H |
| 1641 | 0 | m-D7 | H | A-43 | H | H | H |
| 1642 | 0 | m-D7 | H | A-44 | H | H | H |
| 1643 | 0 | m-D7 | H | A-45 | H | H | H |
| 1644 | 0 | m-D7 | H | A-46 | H | H | H |
| 1645 | 0 | m-D7 | H | A-47 | H | H | H |
| 1646 | 0 | m-D8 | H | A-1 | H | H | H |
| 1647 | 0 | m-D8 | H | A-2 | H | H | H |
| 1648 | 0 | m-D8 | H | A-3 | H | H | H |
| 1649 | 0 | m-D8 | H | A-4 | H | H | H |
| 1650 | 0 | m-D8 | H | A-5 | H | H | H |
| 1651 | 0 | m-D8 | H | A-6 | H | H | H |
| 1652 | 0 | m-D8 | H | A-7 | H | H | H |
| 1653 | 0 | m-D8 | H | A-8 | H | H | H |
| 1654 | 0 | m-D8 | H | A-9 | H | H | H |
| 1655 | 0 | m-D8 | H | A-10 | H | H | H |
| 1656 | 0 | m-D8 | H | A-11 | H | H | H |
| 1657 | 0 | m-D8 | H | A-12 | H | H | H |
| 1658 | 0 | m-D8 | H | A-13 | H | H | H |
| 1659 | 0 | m-D8 | H | A-14 | H | H | H |
| 1660 | 0 | m-D8 | H | A-15 | H | H | H |
| 1661 | 0 | m-D8 | H | A-16 | H | H | H |
| 1662 | 0 | m-D8 | H | A-17 | H | H | H |
| 1663 | 0 | m-D8 | H | A-18 | H | H | H |
| 1664 | 0 | m-D8 | H | A-19 | H | H | H |
| 1665 | 0 | m-D8 | H | A-20 | H | H | H |
| 1666 | 0 | m-D8 | H | A-21 | H | H | H |
| 1667 | 0 | m-D8 | H | A-22 | H | H | H |
| 1668 | 0 | m-D8 | H | A-23 | H | H | H |
| 1669 | 0 | m-D8 | H | A-24 | H | H | H |
| 1670 | 0 | m-D8 | H | A-25 | H | H | H |
| 1671 | 0 | m-D8 | H | A-26 | H | H | H |
| 1672 | 0 | m-D8 | H | A-27 | H | H | H |
| 1673 | 0 | m-D8 | H | A-28 | H | H | H |
| 1674 | 0 | m-D8 | H | A-29 | H | H | H |
| 1675 | 0 | m-D8 | H | A-30 | H | H | H |
| 1676 | 0 | m-D8 | H | A-31 | H | H | H |
| 1677 | 0 | m-D8 | H | A-32 | H | H | H |
| 1678 | 0 | m-D8 | H | A-33 | H | H | H |
| 1679 | 0 | m-D8 | H | A-34 | H | H | H |
| 1680 | 0 | m-D8 | H | A-35 | H | H | H |
| 1681 | 0 | m-D8 | H | A-36 | H | H | H |
| 1682 | 0 | m-D8 | H | A-37 | H | H | H |
| 1683 | 0 | m-D8 | H | A-38 | H | H | H |
| 1684 | 0 | m-D8 | H | A-39 | H | H | H |
| 1685 | 0 | m-D8 | H | A-40 | H | H | H |
| 1686 | 0 | m-D8 | H | A-41 | H | H | H |
| 1687 | 0 | m-D8 | H | A-42 | H | H | H |
| 1688 | 0 | m-D8 | H | A-43 | H | H | H |
| 1689 | 0 | m-D8 | H | A-44 | H | H | H |
| 1690 | 0 | m-D8 | H | A-45 | H | H | H |
| 1691 | 0 | m-D8 | H | A-46 | H | H | H |
| 1692 | 0 | m-D8 | H | A-47 | H | H | H |
| 1693 | 0 | m-D9 | H | A-1 | H | H | H |
| 1694 | 0 | m-D9 | H | A-2 | H | H | H |
| 1695 | 0 | m-D9 | H | A-3 | H | H | H |
| 1696 | 0 | m-D9 | H | A-4 | H | H | H |
| 1697 | 0 | m-D9 | H | A-5 | H | H | H |
| 1698 | 0 | m-D9 | H | A-6 | H | H | H |
| 1699 | 0 | m-D9 | H | A-7 | H | H | H |
| 1700 | 0 | m-D9 | H | A-8 | H | H | H |
| 1701 | 0 | m-D9 | H | A-9 | H | H | H |
| 1702 | 0 | m-D9 | H | A-10 | H | H | H |
| 1703 | 0 | m-D9 | H | A-11 | H | H | H |
| 1704 | 0 | m-D9 | H | A-12 | H | H | H |
| 1705 | 0 | m-D9 | H | A-13 | H | H | H |
| 1706 | 0 | m-D9 | H | A-14 | H | H | H |
| 1707 | 0 | m-D9 | H | A-15 | H | H | H |
| 1708 | 0 | m-D9 | H | A-16 | H | H | H |
| 1709 | 0 | m-D9 | H | A-17 | H | H | H |
| 1710 | 0 | m-D9 | H | A-18 | H | H | H |
| 1711 | 0 | m-D9 | H | A-19 | H | H | H |
| 1712 | 0 | m-D9 | H | A-20 | H | H | H |
| 1713 | 0 | m-D9 | H | A-21 | H | H | H |
| 1714 | 0 | m-D9 | H | A-22 | H | H | H |
| 1715 | 0 | m-D9 | H | A-23 | H | H | H |
| 1716 | 0 | m-D9 | H | A-24 | H | H | H |
| 1717 | 0 | m-D9 | H | A-25 | H | H | H |
| 1718 | 0 | m-D9 | H | A-26 | H | H | H |
| 1719 | 0 | m-D9 | H | A-27 | H | H | H |
| 1720 | 0 | m-D9 | H | A-28 | H | H | H |
| 1721 | 0 | m-D9 | H | A-29 | H | H | H |
| 1722 | 0 | m-D9 | H | A-30 | H | H | H |
| 1723 | 0 | m-D9 | H | A-31 | H | H | H |
| 1724 | 0 | m-D9 | H | A-32 | H | H | H |
| 1725 | 0 | m-D9 | H | A-33 | H | H | H |
| 1726 | 0 | m-D9 | H | A-34 | H | H | H |
| 1727 | 0 | m-D9 | H | A-35 | H | H | H |
| 1728 | 0 | m-D9 | H | A-36 | H | H | H |
| 1729 | 0 | m-D9 | H | A-37 | H | H | H |
| 1730 | 0 | m-D9 | H | A-38 | H | H | H |
| 1731 | 0 | m-D9 | H | A-39 | H | H | H |
| 1732 | 0 | m-D9 | H | A-40 | H | H | H |
| 1733 | 0 | m-D9 | H | A-41 | H | H | H |
| 1734 | 0 | m-D9 | H | A-42 | H | H | H |
| 1735 | 0 | m-D9 | H | A-43 | H | H | H |
| 1736 | 0 | m-D9 | H | A-44 | H | H | H |
| 1737 | 0 | m-D9 | H | A-45 | H | H | H |
| 1738 | 0 | m-D9 | H | A-46 | H | H | H |
| 1739 | 0 | m-D9 | H | A-47 | H | H | H |
| 1740 | 0 | m-D10 | H | A-1 | H | H | H |
| 1741 | 0 | m-D10 | H | A-2 | H | H | H |
| 1742 | 0 | m-D10 | H | A-3 | H | H | H |
| 1743 | 0 | m-D10 | H | A-4 | H | H | H |
| 1744 | 0 | m-D10 | H | A-5 | H | H | H |
| 1745 | 0 | m-D10 | H | A-6 | H | H | H |
| 1746 | 0 | m-D10 | H | A-7 | H | H | H |
| 1747 | 0 | m-D10 | H | A-8 | H | H | H |
| 1748 | 0 | m-D10 | H | A-9 | H | H | H |
| 1749 | 0 | m-D10 | H | A-10 | H | H | H |
| 1750 | 0 | m-D10 | H | A-11 | H | H | H |
| 1751 | 0 | m-D10 | H | A-12 | H | H | H |
| 1752 | 0 | m-D10 | H | A-13 | H | H | H |
| 1753 | 0 | m-D10 | H | A-14 | H | H | H |
| 1754 | 0 | m-D10 | H | A-15 | H | H | H |
| 1755 | 0 | m-D10 | H | A-16 | H | H | H |
| 1756 | 0 | m-D10 | H | A-17 | H | H | H |
| 1757 | 0 | m-D10 | H | A-18 | H | H | H |
| 1758 | 0 | m-D10 | H | A-19 | H | H | H |
| 1759 | 0 | m-D10 | H | A-20 | H | H | H |
| 1760 | 0 | m-D10 | H | A-21 | H | H | H |
| 1761 | 0 | m-D10 | H | A-22 | H | H | H |
| 1762 | 0 | m-D10 | H | A-23 | H | H | H |
| 1763 | 0 | m-D10 | H | A-24 | H | H | H |
| 1764 | 0 | m-D10 | H | A-25 | H | H | H |
| 1765 | 0 | m-D10 | H | A-26 | H | H | H |
| 1766 | 0 | m-D10 | H | A-27 | H | H | H |
| 1767 | 0 | m-D10 | H | A-28 | H | H | H |
| 1768 | 0 | m-D10 | H | A-29 | H | H | H |
| 1769 | 0 | m-D10 | H | A-30 | H | H | H |
| 1770 | 0 | m-D10 | H | A-31 | H | H | H |
| 1771 | 0 | m-D10 | H | A-32 | H | H | H |
| 1772 | 0 | m-D10 | H | A-33 | H | H | H |
| 1773 | 0 | m-D10 | H | A-34 | H | H | H |
| 1774 | 0 | m-D10 | H | A-35 | H | H | H |
| 1775 | 0 | m-D10 | H | A-36 | H | H | H |
| 1776 | 0 | m-D10 | H | A-37 | H | H | H |
| 1777 | 0 | m-D10 | H | A-38 | H | H | H |
| 1778 | 0 | m-D10 | H | A-39 | H | H | H |
| 1779 | 0 | m-D10 | H | A-40 | H | H | H |
| 1780 | 0 | m-D10 | H | A-41 | H | H | H |
| 1781 | 0 | m-D10 | H | A-42 | H | H | H |
| 1782 | 0 | m-D10 | H | A-43 | H | H | H |
| 1783 | 0 | m-D10 | H | A-44 | H | H | H |
| 1784 | 0 | m-D10 | H | A-45 | H | H | H |
| 1785 | 0 | m-D10 | H | A-46 | H | H | H |
| 1786 | 0 | m-D10 | H | A-47 | H | H | H |
| 1787 | 0 | m-D11 | H | A-1 | H | H | H |
| 1788 | 0 | m-D11 | H | A-2 | H | H | H |
| 1789 | 0 | m-D11 | H | A-3 | H | H | H |
| 1790 | 0 | m-D11 | H | A-4 | H | H | H |
| 1791 | 0 | m-D11 | H | A-5 | H | H | H |
| 1792 | 0 | m-D11 | H | A-6 | H | H | H |
| 1793 | 0 | m-D11 | H | A-7 | H | H | H |
| 1794 | 0 | m-D11 | H | A-8 | H | H | H |
| 1795 | 0 | m-D11 | H | A-9 | H | H | H |
| 1796 | 0 | m-D11 | H | A-10 | H | H | H |
| 1797 | 0 | m-D11 | H | A-11 | H | H | H |
| 1798 | 0 | m-D11 | H | A-12 | H | H | H |
| 1799 | 0 | m-D11 | H | A-13 | H | H | H |
| 1800 | 0 | m-D11 | H | A-14 | H | H | H |
| 1801 | 0 | m-D11 | H | A-15 | H | H | H |
| 1802 | 0 | m-D11 | H | A-16 | H | H | H |
| 1803 | 0 | m-D11 | H | A-17 | H | H | H |
| 1804 | 0 | m-D11 | H | A-18 | H | H | H |
| 1805 | 0 | m-D11 | H | A-19 | H | H | H |
| 1806 | 0 | m-D11 | H | A-20 | H | H | H |
| 1807 | 0 | m-D11 | H | A-21 | H | H | H |
| 1808 | 0 | m-D11 | H | A-22 | H | H | H |
| 1809 | 0 | m-D11 | H | A-23 | H | H | H |
| 1810 | 0 | m-D11 | H | A-24 | H | H | H |
| 1811 | 0 | m-D11 | H | A-25 | H | H | H |
| 1812 | 0 | m-D11 | H | A-26 | H | H | H |
| 1813 | 0 | m-D11 | H | A-27 | H | H | H |
| 1814 | 0 | m-D11 | H | A-28 | H | H | H |
| 1815 | 0 | m-D11 | H | A-29 | H | H | H |
| 1816 | 0 | m-D11 | H | A-30 | H | H | H |
| 1817 | 0 | m-D11 | H | A-31 | H | H | H |
| 1818 | 0 | m-D11 | H | A-32 | H | H | H |
| 1819 | 0 | m-D11 | H | A-33 | H | H | H |
| 1820 | 0 | m-D11 | H | A-34 | H | H | H |
| 1821 | 0 | m-D11 | H | A-35 | H | H | H |
| 1822 | 0 | m-D11 | H | A-36 | H | H | H |
| 1823 | 0 | m-D11 | H | A-37 | H | H | H |
| 1824 | 0 | m-D11 | H | A-38 | H | H | H |
| 1825 | 0 | m-D11 | H | A-39 | H | H | H |
| 1826 | 0 | m-D11 | H | A-40 | H | H | H |
| 1827 | 0 | m-D11 | H | A-41 | H | H | H |
| 1828 | 0 | m-D11 | H | A-42 | H | H | H |
| 1829 | 0 | m-D11 | H | A-43 | H | H | H |
| 1830 | 0 | m-D11 | H | A-44 | H | H | H |
| 1831 | 0 | m-D11 | H | A-45 | H | H | H |
| 1832 | 0 | m-D11 | H | A-46 | H | H | H |
| 1833 | 0 | m-D11 | H | A-47 | H | H | H |
| 1834 | 0 | m-D12 | H | A-1 | H | H | H |
| 1835 | 0 | m-D12 | H | A-2 | H | H | H |
| 1836 | 0 | m-D12 | H | A-3 | H | H | H |
| 1837 | 0 | m-D12 | H | A-4 | H | H | H |
| 1838 | 0 | m-D12 | H | A-5 | H | H | H |
| 1839 | 0 | m-D12 | H | A-6 | H | H | H |
| 1840 | 0 | m-D12 | H | A-7 | H | H | H |
| 1841 | 0 | m-D12 | H | A-8 | H | H | H |
| 1842 | 0 | m-D12 | H | A-9 | H | H | H |
| 1843 | 0 | m-D12 | H | A-10 | H | H | H |
| 1844 | 0 | m-D12 | H | A-11 | H | H | H |
| 1845 | 0 | m-D12 | H | A-12 | H | H | H |
| 1846 | 0 | m-D12 | H | A-13 | H | H | H |
| 1847 | 0 | m-D12 | H | A-14 | H | H | H |
| 1848 | 0 | m-D12 | H | A-15 | H | H | H |
| 1849 | 0 | m-D12 | H | A-16 | H | H | H |
| 1850 | 0 | m-D12 | H | A-17 | H | H | H |
| 1851 | 0 | m-D12 | H | A-18 | H | H | H |
| 1852 | 0 | m-D12 | H | A-19 | H | H | H |
| 1853 | 0 | m-D12 | H | A-20 | H | H | H |
| 1854 | 0 | m-D12 | H | A-21 | H | H | H |
| 1855 | 0 | m-D12 | H | A-22 | H | H | H |
| 1856 | 0 | m-D12 | H | A-23 | H | H | H |
| 1857 | 0 | m-D12 | H | A-24 | H | H | H |
| 1858 | 0 | m-D12 | H | A-25 | H | H | H |
| 1859 | 0 | m-D12 | H | A-26 | H | H | H |
| 1860 | 0 | m-D12 | H | A-27 | H | H | H |
| 1861 | 0 | m-D12 | H | A-28 | H | H | H |
| 1862 | 0 | m-D12 | H | A-29 | H | H | H |
| 1863 | 0 | m-D12 | H | A-30 | H | H | H |
| 1864 | 0 | m-D12 | H | A-31 | H | H | H |
| 1865 | 0 | m-D12 | H | A-32 | H | H | H |
| 1866 | 0 | m-D12 | H | A-33 | H | H | H |
| 1867 | 0 | m-D12 | H | A-34 | H | H | H |
| 1868 | 0 | m-D12 | H | A-35 | H | H | H |
| 1869 | 0 | m-D12 | H | A-36 | H | H | H |
| 1870 | 0 | m-D12 | H | A-37 | H | H | H |
| 1871 | 0 | m-D12 | H | A-38 | H | H | H |
| 1872 | 0 | m-D12 | H | A-39 | H | H | H |
| 1873 | 0 | m-D12 | H | A-40 | H | H | H |
| 1874 | 0 | m-D12 | H | A-41 | H | H | H |
| 1875 | 0 | m-D12 | H | A-42 | H | H | H |
| 1876 | 0 | m-D12 | H | A-43 | H | H | H |
| 1877 | 0 | m-D12 | H | A-44 | H | H | H |
| 1878 | 0 | m-D12 | H | A-45 | H | H | H |
| 1879 | 0 | m-D12 | H | A-46 | H | H | H |
| 1880 | 0 | m-D12 | H | A-47 | H | H | H |
| 1881 | 0 | m-D13 | H | A-1 | H | H | H |
| 1882 | 0 | m-D13 | H | A-2 | H | H | H |
| 1883 | 0 | m-D13 | H | A-3 | H | H | H |
| 1884 | 0 | m-D13 | H | A-4 | H | H | H |
| 1885 | 0 | m-D13 | H | A-5 | H | H | H |
| 1886 | 0 | m-D13 | H | A-6 | H | H | H |
| 1887 | 0 | m-D13 | H | A-7 | H | H | H |
| 1888 | 0 | m-D13 | H | A-8 | H | H | H |
| 1889 | 0 | m-D13 | H | A-9 | H | H | H |
| 1890 | 0 | m-D13 | H | A-10 | H | H | H |
| 1891 | 0 | m-D13 | H | A-11 | H | H | H |
| 1892 | 0 | m-D13 | H | A-12 | H | H | H |
| 1893 | 0 | m-D13 | H | A-13 | H | H | H |
| 1894 | 0 | m-D13 | H | A-14 | H | H | H |
| 1895 | 0 | m-D13 | H | A-15 | H | H | H |
| 1896 | 0 | m-D13 | H | A-16 | H | H | H |
| 1897 | 0 | m-D13 | H | A-17 | H | H | H |
| 1898 | 0 | m-D13 | H | A-18 | H | H | H |
| 1899 | 0 | m-D13 | H | A-19 | H | H | H |
| 1900 | 0 | m-D13 | H | A-20 | H | H | H |
| 1901 | 0 | m-D13 | H | A-21 | H | H | H |
| 1902 | 0 | m-D13 | H | A-22 | H | H | H |
| 1903 | 0 | m-D13 | H | A-23 | H | H | H |
| 1904 | 0 | m-D13 | H | A-24 | H | H | H |
| 1905 | 0 | m-D13 | H | A-25 | H | H | H |
| 1906 | 0 | m-D13 | H | A-26 | H | H | H |
| 1907 | 0 | m-D13 | H | A-27 | H | H | H |
| 1908 | 0 | m-D13 | H | A-28 | H | H | H |
| 1909 | 0 | m-D13 | H | A-29 | H | H | H |
| 1910 | 0 | m-D13 | H | A-30 | H | H | H |
| 1911 | 0 | m-D13 | H | A-31 | H | H | H |
| 1912 | 0 | m-D13 | H | A-32 | H | H | H |
| 1913 | 0 | m-D13 | H | A-33 | H | H | H |
| 1914 | 0 | m-D13 | H | A-34 | H | H | H |
| 1915 | 0 | m-D13 | H | A-35 | H | H | H |
| 1916 | 0 | m-D13 | H | A-36 | H | H | H |
| 1917 | 0 | m-D13 | H | A-37 | H | H | H |
| 1918 | 0 | m-D13 | H | A-38 | H | H | H |
| 1919 | 0 | m-D13 | H | A-39 | H | H | H |
| 1920 | 0 | m-D13 | H | A-40 | H | H | H |
| 1921 | 0 | m-D13 | H | A-41 | H | H | H |
| 1922 | 0 | m-D13 | H | A-42 | H | H | H |
| 1923 | 0 | m-D13 | H | A-43 | H | H | H |
| 1924 | 0 | m-D13 | H | A-44 | H | H | H |
| 1925 | 0 | m-D13 | H | A-45 | H | H | H |
| 1926 | 0 | m-D13 | H | A-46 | H | H | H |
| 1927 | 0 | m-D13 | H | A-47 | H | H | H |
| 1928 | 0 | m-D14 | H | A-1 | H | H | H |
| 1929 | 0 | m-D14 | H | A-2 | H | H | H |
| 1930 | 0 | m-D14 | H | A-3 | H | H | H |
| 1931 | 0 | m-D14 | H | A-4 | H | H | H |
| 1932 | 0 | m-D14 | H | A-5 | H | H | H |
| 1933 | 0 | m-D14 | H | A-6 | H | H | H |
| 1934 | 0 | m-D14 | H | A-7 | H | H | H |
| 1935 | 0 | m-D14 | H | A-8 | H | H | H |
| 1936 | 0 | m-D14 | H | A-9 | H | H | H |
| 1937 | 0 | m-D14 | H | A-10 | H | H | H |
| 1938 | 0 | m-D14 | H | A-11 | H | H | H |
| 1939 | 0 | m-D14 | H | A-12 | H | H | H |
| 1940 | 0 | m-D14 | H | A-13 | H | H | H |
| 1941 | 0 | m-D14 | H | A-14 | H | H | H |
| 1942 | 0 | m-D14 | H | A-15 | H | H | H |
| 1943 | 0 | m-D14 | H | A-16 | H | H | H |
| 1944 | 0 | m-D14 | H | A-17 | H | H | H |
| 1945 | 0 | m-D14 | H | A-18 | H | H | H |
| 1946 | 0 | m-D14 | H | A-19 | H | H | H |
| 1947 | 0 | m-D14 | H | A-20 | H | H | H |
| 1948 | 0 | m-D14 | H | A-21 | H | H | H |
| 1949 | 0 | m-D14 | H | A-22 | H | H | H |
| 1950 | 0 | m-D14 | H | A-23 | H | H | H |
| 1951 | 0 | m-D14 | H | A-24 | H | H | H |
| 1952 | 0 | m-D14 | H | A-25 | H | H | H |
| 1953 | 0 | m-D14 | H | A-26 | H | H | H |
| 1954 | 0 | m-D14 | H | A-27 | H | H | H |
| 1955 | 0 | m-D14 | H | A-28 | H | H | H |
| 1956 | 0 | m-D14 | H | A-29 | H | H | H |
| 1957 | 0 | m-D14 | H | A-30 | H | H | H |
| 1958 | 0 | m-D14 | H | A-31 | H | H | H |
| 1959 | 0 | m-D14 | H | A-32 | H | H | H |
| 1960 | 0 | m-D14 | H | A-33 | H | H | H |
| 1961 | 0 | m-D14 | H | A-34 | H | H | H |
| 1962 | 0 | m-D14 | H | A-35 | H | H | H |
| 1963 | 0 | m-D14 | H | A-36 | H | H | H |
| 1964 | 0 | m-D14 | H | A-37 | H | H | H |
| 1965 | 0 | m-D14 | H | A-38 | H | H | H |
| 1966 | 0 | m-D14 | H | A-39 | H | H | H |
| 1967 | 0 | m-D14 | H | A-40 | H | H | H |
| 1968 | 0 | m-D14 | H | A-41 | H | H | H |
| 1969 | 0 | m-D14 | H | A-42 | H | H | H |
| 1970 | 0 | m-D14 | H | A-43 | H | H | H |
| 1971 | 0 | m-D14 | H | A-44 | H | H | H |
| 1972 | 0 | m-D14 | H | A-45 | H | H | H |
| 1973 | 0 | m-D14 | H | A-46 | H | H | H |
| 1974 | 0 | m-D14 | H | A-47 | H | H | H |
| 1975 | 0 | m-D1 | H | A-1 | H | A-1 | H |
| 1976 | 0 | m-D1 | H | A-2 | H | A-2 | H |
| 1977 | 0 | m-D1 | H | A-3 | H | A-3 | H |
| 1978 | 0 | m-D1 | H | A-4 | H | A-4 | H |
| 1979 | 0 | m-D1 | H | A-5 | H | A-5 | H |
| 1980 | 0 | m-D1 | H | A-6 | H | A-6 | H |
| 1981 | 0 | m-D1 | H | A-7 | H | A-7 | H |
| 1982 | 0 | m-D1 | H | A-8 | H | A-8 | H |
| 1983 | 0 | m-D1 | H | A-9 | H | A-9 | H |
| 1984 | 0 | m-D1 | H | A-10 | H | A-10 | H |
| 1985 | 0 | m-D1 | H | A-11 | H | A-11 | H |
| 1986 | 0 | m-D1 | H | A-12 | H | A-12 | H |
| 1987 | 0 | m-D1 | H | A-13 | H | A-13 | H |
| 1988 | 0 | m-D1 | H | A-14 | H | A-14 | H |
| 1989 | 0 | m-D1 | H | A-15 | H | A-15 | H |
| 1990 | 0 | m-D1 | H | A-16 | H | A-16 | H |
| 1991 | 0 | m-D1 | H | A-17 | H | A-17 | H |
| 1992 | 0 | m-D1 | H | A-18 | H | A-18 | H |
| 1993 | 0 | m-D1 | H | A-19 | H | A-19 | H |
| 1994 | 0 | m-D1 | H | A-20 | H | A-20 | H |
| 1995 | 0 | m-D1 | H | A-21 | H | A-21 | H |
| 1996 | 0 | m-D1 | H | A-22 | H | A-22 | H |
| 1997 | 0 | m-D1 | H | A-23 | H | A-23 | H |
| 1998 | 0 | m-D1 | H | A-24 | H | A-24 | H |
| 1999 | 0 | m-D1 | H | A-25 | H | A-25 | H |
| 2000 | 0 | m-D1 | H | A-26 | H | A-26 | H |
| 2001 | 0 | m-D1 | H | A-27 | H | A-27 | H |
| 2002 | 0 | m-D1 | H | A-28 | H | A-28 | H |
| 2003 | 0 | m-D1 | H | A-29 | H | A-29 | H |
| 2004 | 0 | m-D1 | H | A-30 | H | A-30 | H |
| 2005 | 0 | m-D1 | H | A-31 | H | A-31 | H |
| 2006 | 0 | m-D1 | H | A-32 | H | A-32 | H |
| 2007 | 0 | m-D1 | H | A-33 | H | A-33 | H |
| 2008 | 0 | m-D1 | H | A-34 | H | A-34 | H |
| 2009 | 0 | m-D1 | H | A-35 | H | A-35 | H |
| 2010 | 0 | m-D1 | H | A-36 | H | A-36 | H |
| 2011 | 0 | m-D1 | H | A-37 | H | A-37 | H |
| 2012 | 0 | m-D1 | H | A-38 | H | A-38 | H |
| 2013 | 0 | m-D1 | H | A-39 | H | A-39 | H |
| 2014 | 0 | m-D1 | H | A-40 | H | A-40 | H |
| 2015 | 0 | m-D1 | H | A-41 | H | A-41 | H |
| 2016 | 0 | m-D1 | H | A-42 | H | A-42 | H |
| 2017 | 0 | m-D1 | H | A-43 | H | A-43 | H |
| 2018 | 0 | m-D1 | H | A-44 | H | A-44 | H |
| 2019 | 0 | m-D1 | H | A-45 | H | A-45 | H |
| 2020 | 0 | m-D1 | H | A-46 | H | A-46 | H |
| 2021 | 0 | m-D1 | H | A-47 | H | A-47 | H |
| 2022 | 0 | m-D2 | H | A-1 | H | A-1 | H |
| 2023 | 0 | m-D2 | H | A-2 | H | A-2 | H |
| 2024 | 0 | m-D2 | H | A-3 | H | A-3 | H |
| 2025 | 0 | m-D2 | H | A-4 | H | A-4 | H |
| 2026 | 0 | m-D2 | H | A-5 | H | A-5 | H |
| 2027 | 0 | m-D2 | H | A-6 | H | A-6 | H |
| 2028 | 0 | m-D2 | H | A-7 | H | A-7 | H |
| 2029 | 0 | m-D2 | H | A-8 | H | A-8 | H |
| 2030 | 0 | m-D2 | H | A-9 | H | A-9 | H |
| 2031 | 0 | m-D2 | H | A-10 | H | A-10 | H |
| 2032 | 0 | m-D2 | H | A-11 | H | A-11 | H |
| 2033 | 0 | m-D2 | H | A-12 | H | A-12 | H |
| 2034 | 0 | m-D2 | H | A-13 | H | A-13 | H |
| 2035 | 0 | m-D2 | H | A-14 | H | A-14 | H |
| 2036 | 0 | m-D2 | H | A-15 | H | A-15 | H |
| 2037 | 0 | m-D2 | H | A-16 | H | A-16 | H |
| 2038 | 0 | m-D2 | H | A-17 | H | A-17 | H |
| 2039 | 0 | m-D2 | H | A-18 | H | A-18 | H |
| 2040 | 0 | m-D2 | H | A-19 | H | A-19 | H |
| 2041 | 0 | m-D2 | H | A-20 | H | A-20 | H |
| 2042 | 0 | m-D2 | H | A-21 | H | A-21 | H |
| 2043 | 0 | m-D2 | H | A-22 | H | A-22 | H |
| 2044 | 0 | m-D2 | H | A-23 | H | A-23 | H |
| 2045 | 0 | m-D2 | H | A-24 | H | A-24 | H |
| 2046 | 0 | m-D2 | H | A-25 | H | A-25 | H |
| 2047 | 0 | m-D2 | H | A-26 | H | A-26 | H |
| 2048 | 0 | m-D2 | H | A-27 | H | A-27 | H |
| 2049 | 0 | m-D2 | H | A-28 | H | A-28 | H |
| 2050 | 0 | m-D2 | H | A-29 | H | A-29 | H |
| 2051 | 0 | m-D2 | H | A-30 | H | A-30 | H |
| 2052 | 0 | m-D2 | H | A-31 | H | A-31 | H |
| 2053 | 0 | m-D2 | H | A-32 | H | A-32 | H |
| 2054 | 0 | m-D2 | H | A-33 | H | A-33 | H |
| 2055 | 0 | m-D2 | H | A-34 | H | A-34 | H |
| 2056 | 0 | m-D2 | H | A-35 | H | A-35 | H |
| 2057 | 0 | m-D2 | H | A-36 | H | A-36 | H |
| 2058 | 0 | m-D2 | H | A-37 | H | A-37 | H |
| 2059 | 0 | m-D2 | H | A-38 | H | A-38 | H |
| 2060 | 0 | m-D2 | H | A-39 | H | A-39 | H |
| 2061 | 0 | m-D2 | H | A-40 | H | A-40 | H |
| 2062 | 0 | m-D2 | H | A-41 | H | A-41 | H |
| 2063 | 0 | m-D2 | H | A-42 | H | A-42 | H |
| 2064 | 0 | m-D2 | H | A-43 | H | A-43 | H |
| 2065 | 0 | m-D2 | H | A-44 | H | A-44 | H |
| 2066 | 0 | m-D2 | H | A-45 | H | A-45 | H |
| 2067 | 0 | m-D2 | H | A-46 | H | A-46 | H |
| 2068 | 0 | m-D2 | H | A-47 | H | A-47 | H |
| 2069 | 0 | m-D3 | H | A-1 | H | A-1 | H |
| 2070 | 0 | m-D3 | H | A-2 | H | A-2 | H |
| 2071 | 0 | m-D3 | H | A-3 | H | A-3 | H |
| 2072 | 0 | m-D3 | H | A-4 | H | A-4 | H |
| 2073 | 0 | m-D3 | H | A-5 | H | A-5 | H |
| 2074 | 0 | m-D3 | H | A-6 | H | A-6 | H |
| 2075 | 0 | m-D3 | H | A-7 | H | A-7 | H |
| 2076 | 0 | m-D3 | H | A-8 | H | A-8 | H |
| 2077 | 0 | m-D3 | H | A-9 | H | A-9 | H |
| 2078 | 0 | m-D3 | H | A-10 | H | A-10 | H |
| 2079 | 0 | m-D3 | H | A-11 | H | A-11 | H |
| 2080 | 0 | m-D3 | H | A-12 | H | A-12 | H |
| 2081 | 0 | m-D3 | H | A-13 | H | A-13 | H |
| 2082 | 0 | m-D3 | H | A-14 | H | A-14 | H |
| 2083 | 0 | m-D3 | H | A-15 | H | A-15 | H |
| 2084 | 0 | m-D3 | H | A-16 | H | A-16 | H |
| 2085 | 0 | m-D3 | H | A-17 | H | A-17 | H |
| 2086 | 0 | m-D3 | H | A-18 | H | A-18 | H |
| 2087 | 0 | m-D3 | H | A-19 | H | A-19 | H |
| 2088 | 0 | m-D3 | H | A-20 | H | A-20 | H |
| 2089 | 0 | m-D3 | H | A-21 | H | A-21 | H |
| 2090 | 0 | m-D3 | H | A-22 | H | A-22 | H |
| 2091 | 0 | m-D3 | H | A-23 | H | A-23 | H |
| 2092 | 0 | m-D3 | H | A-24 | H | A-24 | H |
| 2093 | 0 | m-D3 | H | A-25 | H | A-25 | H |
| 2094 | 0 | m-D3 | H | A-26 | H | A-26 | H |
| 2095 | 0 | m-D3 | H | A-27 | H | A-27 | H |
| 2096 | 0 | m-D3 | H | A-28 | H | A-28 | H |
| 2097 | 0 | m-D3 | H | A-29 | H | A-29 | H |
| 2098 | 0 | m-D3 | H | A-30 | H | A-30 | H |
| 2099 | 0 | m-D3 | H | A-31 | H | A-31 | H |
| 2100 | 0 | m-D3 | H | A-32 | H | A-32 | H |
| 2101 | 0 | m-D3 | H | A-33 | H | A-33 | H |
| 2102 | 0 | m-D3 | H | A-34 | H | A-34 | H |
| 2103 | 0 | m-D3 | H | A-35 | H | A-35 | H |
| 2104 | 0 | m-D3 | H | A-36 | H | A-36 | H |
| 2105 | 0 | m-D3 | H | A-37 | H | A-37 | H |
| 2106 | 0 | m-D3 | H | A-38 | H | A-38 | H |
| 2107 | 0 | m-D3 | H | A-39 | H | A-39 | H |
| 2108 | 0 | m-D3 | H | A-40 | H | A-40 | H |
| 2109 | 0 | m-D3 | H | A-41 | H | A-41 | H |
| 2110 | 0 | m-D3 | H | A-42 | H | A-42 | H |
| 2111 | 0 | m-D3 | H | A-43 | H | A-43 | H |
| 2112 | 0 | m-D3 | H | A-44 | H | A-44 | H |
| 2113 | 0 | m-D3 | H | A-45 | H | A-45 | H |
| 2114 | 0 | m-D3 | H | A-46 | H | A-46 | H |
| 2115 | 0 | m-D3 | H | A-47 | H | A-47 | H |
| 2116 | 0 | m-D5 | H | A-1 | H | A-1 | H |
| 2117 | 0 | m-D5 | H | A-2 | H | A-2 | H |
| 2118 | 0 | m-D5 | H | A-3 | H | A-3 | H |
| 2119 | 0 | m-D5 | H | A-4 | H | A-4 | H |
| 2120 | 0 | m-D5 | H | A-5 | H | A-5 | H |
| 2121 | 0 | m-D5 | H | A-6 | H | A-6 | H |
| 2122 | 0 | m-D5 | H | A-7 | H | A-7 | H |
| 2123 | 0 | m-D5 | H | A-8 | H | A-8 | H |
| 2124 | 0 | m-D5 | H | A-9 | H | A-9 | H |
| 2125 | 0 | m-D5 | H | A-10 | H | A-10 | H |
| 2126 | 0 | m-D5 | H | A-11 | H | A-11 | H |
| 2127 | 0 | m-D5 | H | A-12 | H | A-12 | H |
| 2128 | 0 | m-D5 | H | A-13 | H | A-13 | H |
| 2129 | 0 | m-D5 | H | A-14 | H | A-14 | H |
| 2130 | 0 | m-D5 | H | A-15 | H | A-15 | H |
| 2131 | 0 | m-D5 | H | A-16 | H | A-16 | H |
| 2132 | 0 | m-D5 | H | A-17 | H | A-17 | H |
| 2133 | 0 | m-D5 | H | A-18 | H | A-18 | H |
| 2134 | 0 | m-D5 | H | A-19 | H | A-19 | H |
| 2135 | 0 | m-D5 | H | A-20 | H | A-20 | H |
| 2136 | 0 | m-D5 | H | A-21 | H | A-21 | H |
| 2137 | 0 | m-D5 | H | A-22 | H | A-22 | H |
| 2138 | 0 | m-D5 | H | A-23 | H | A-23 | H |
| 2139 | 0 | m-D5 | H | A-24 | H | A-24 | H |
| 2140 | 0 | m-D5 | H | A-25 | H | A-25 | H |
| 2141 | 0 | m-D5 | H | A-26 | H | A-26 | H |
| 2142 | 0 | m-D5 | H | A-27 | H | A-27 | H |
| 2143 | 0 | m-D5 | H | A-28 | H | A-28 | H |
| 2144 | 0 | m-D5 | H | A-29 | H | A-29 | H |
| 2145 | 0 | m-D5 | H | A-30 | H | A-30 | H |
| 2146 | 0 | m-D5 | H | A-31 | H | A-31 | H |
| 2147 | 0 | m-D5 | H | A-32 | H | A-32 | H |
| 2148 | 0 | m-D5 | H | A-33 | H | A-33 | H |
| 2149 | 0 | m-D5 | H | A-34 | H | A-34 | H |
| 2150 | 0 | m-D5 | H | A-35 | H | A-35 | H |
| 2151 | 0 | m-D5 | H | A-36 | H | A-36 | H |
| 2152 | 0 | m-D5 | H | A-37 | H | A-37 | H |
| 2153 | 0 | m-D5 | H | A-38 | H | A-38 | H |
| 2154 | 0 | m-D5 | H | A-39 | H | A-39 | H |
| 2155 | 0 | m-D5 | H | A-40 | H | A-40 | H |
| 2156 | 0 | m-D5 | H | A-41 | H | A-41 | H |
| 2157 | 0 | m-D5 | H | A-42 | H | A-42 | H |
| 2158 | 0 | m-D5 | H | A-43 | H | A-43 | H |
| 2159 | 0 | m-D5 | H | A-44 | H | A-44 | H |
| 2160 | 0 | m-D5 | H | A-45 | H | A-45 | H |
| 2161 | 0 | m-D5 | H | A-46 | H | A-46 | H |
| 2162 | 0 | m-D5 | H | A-47 | H | A-47 | H |
| 2163 | 0 | m-D9 | H | A-1 | H | A-1 | H |
| 2164 | 0 | m-D9 | H | A-2 | H | A-2 | H |
| 2165 | 0 | m-D9 | H | A-3 | H | A-3 | H |
| 2166 | 0 | m-D9 | H | A-4 | H | A-4 | H |
| 2167 | 0 | m-D9 | H | A-5 | H | A-5 | H |
| 2168 | 0 | m-D9 | H | A-6 | H | A-6 | H |
| 2169 | 0 | m-D9 | H | A-7 | H | A-7 | H |
| 2170 | 0 | m-D9 | H | A-8 | H | A-8 | H |
| 2171 | 0 | m-D9 | H | A-9 | H | A-9 | H |
| 2172 | 0 | m-D9 | H | A-10 | H | A-10 | H |
| 2173 | 0 | m-D9 | H | A-11 | H | A-11 | H |
| 2174 | 0 | m-D9 | H | A-12 | H | A-12 | H |
| 2175 | 0 | m-D9 | H | A-13 | H | A-13 | H |
| 2176 | 0 | m-D9 | H | A-14 | H | A-14 | H |
| 2177 | 0 | m-D9 | H | A-15 | H | A-15 | H |
| 2178 | 0 | m-D9 | H | A-16 | H | A-16 | H |
| 2179 | 0 | m-D9 | H | A-17 | H | A-17 | H |
| 2180 | 0 | m-D9 | H | A-18 | H | A-18 | H |
| 2181 | 0 | m-D9 | H | A-19 | H | A-19 | H |
| 2182 | 0 | m-D9 | H | A-20 | H | A-20 | H |
| 2183 | 0 | m-D9 | H | A-21 | H | A-21 | H |
| 2184 | 0 | m-D9 | H | A-22 | H | A-22 | H |
| 2185 | 0 | m-D9 | H | A-23 | H | A-23 | H |
| 2186 | 0 | m-D9 | H | A-24 | H | A-24 | H |
| 2187 | 0 | m-D9 | H | A-25 | H | A-25 | H |
| 2188 | 0 | m-D9 | H | A-26 | H | A-26 | H |
| 2189 | 0 | m-D9 | H | A-27 | H | A-27 | H |
| 2190 | 0 | m-D9 | H | A-28 | H | A-28 | H |
| 2191 | 0 | m-D9 | H | A-29 | H | A-29 | H |
| 2192 | 0 | m-D9 | H | A-30 | H | A-30 | H |
| 2193 | 0 | m-D9 | H | A-31 | H | A-31 | H |
| 2194 | 0 | m-D9 | H | A-32 | H | A-32 | H |
| 2195 | 0 | m-D9 | H | A-33 | H | A-33 | H |
| 2196 | 0 | m-D9 | H | A-34 | H | A-34 | H |
| 2197 | 0 | m-D9 | H | A-35 | H | A-35 | H |
| 2198 | 0 | m-D9 | H | A-36 | H | A-36 | H |
| 2199 | 0 | m-D9 | H | A-37 | H | A-37 | H |
| 2200 | 0 | m-D9 | H | A-38 | H | A-38 | H |
| 2201 | 0 | m-D9 | H | A-39 | H | A-39 | H |
| 2202 | 0 | m-D9 | H | A-40 | H | A-40 | H |
| 2203 | 0 | m-D9 | H | A-41 | H | A-41 | H |
| 2204 | 0 | m-D9 | H | A-42 | H | A-42 | H |
| 2205 | 0 | m-D9 | H | A-43 | H | A-43 | H |
| 2206 | 0 | m-D9 | H | A-44 | H | A-44 | H |
| 2207 | 0 | m-D9 | H | A-45 | H | A-45 | H |
| 2208 | 0 | m-D9 | H | A-46 | H | A-46 | H |
| 2209 | 0 | m-D11 | H | A-47 | H | A-47 | H |
| 2210 | 0 | m-D11 | H | A-1 | H | A-1 | H |
| 2211 | 0 | m-D11 | H | A-2 | H | A-2 | H |
| 2212 | 0 | m-D11 | H | A-3 | H | A-3 | H |
| 2213 | 0 | m-D11 | H | A-4 | H | A-4 | H |
| 2214 | 0 | m-D11 | H | A-5 | H | A-5 | H |
| 2215 | 0 | m-D11 | H | A-6 | H | A-6 | H |
| 2216 | 0 | m-D11 | H | A-7 | H | A-7 | H |
| 2217 | 0 | m-D11 | H | A-8 | H | A-8 | H |
| 2218 | 0 | m-D11 | H | A-9 | H | A-9 | H |
| 2219 | 0 | m-D11 | H | A-10 | H | A-10 | H |
| 2220 | 0 | m-D11 | H | A-11 | H | A-11 | H |
| 2221 | 0 | m-D11 | H | A-12 | H | A-12 | H |
| 2222 | 0 | m-D11 | H | A-13 | H | A-13 | H |
| 2223 | 0 | m-D11 | H | A-14 | H | A-14 | H |
| 2224 | 0 | m-D11 | H | A-15 | H | A-15 | H |
| 2225 | 0 | m-D11 | H | A-16 | H | A-16 | H |
| 2226 | 0 | m-D11 | H | A-17 | H | A-17 | H |
| 2227 | 0 | m-D11 | H | A-18 | H | A-18 | H |
| 2228 | 0 | m-D11 | H | A-19 | H | A-19 | H |
| 2229 | 0 | m-D11 | H | A-20 | H | A-20 | H |
| 2230 | 0 | m-D11 | H | A-21 | H | A-21 | H |
| 2231 | 0 | m-D11 | H | A-22 | H | A-22 | H |
| 2232 | 0 | m-D11 | H | A-23 | H | A-23 | H |
| 2233 | 0 | m-D11 | H | A-24 | H | A-24 | H |
| 2234 | 0 | m-D11 | H | A-25 | H | A-25 | H |
| 2235 | 0 | m-D11 | H | A-26 | H | A-26 | H |
| 2236 | 0 | m-D11 | H | A-27 | H | A-27 | H |
| 2237 | 0 | m-D11 | H | A-28 | H | A-28 | H |
| 2238 | 0 | m-D11 | H | A-29 | H | A-29 | H |
| 2239 | 0 | m-D11 | H | A-30 | H | A-30 | H |
| 2240 | 0 | m-D11 | H | A-31 | H | A-31 | H |
| 2241 | 0 | m-D11 | H | A-32 | H | A-32 | H |
| 2242 | 0 | m-D11 | H | A-33 | H | A-33 | H |
| 2243 | 0 | m-D11 | H | A-34 | H | A-34 | H |
| 2244 | 0 | m-D11 | H | A-35 | H | A-35 | H |
| 2245 | 0 | m-D11 | H | A-36 | H | A-36 | H |
| 2246 | 0 | m-D11 | H | A-37 | H | A-37 | H |
| 2247 | 0 | m-D11 | H | A-38 | H | A-38 | H |
| 2248 | 0 | m-D11 | H | A-39 | H | A-39 | H |
| 2249 | 0 | m-D11 | H | A-40 | H | A-40 | H |
| 2250 | 0 | m-D11 | H | A-41 | H | A-41 | H |
| 2251 | 0 | m-D11 | H | A-42 | H | A-42 | H |
| 2252 | 0 | m-D11 | H | A-43 | H | A-43 | H |
| 2253 | 0 | m-D11 | H | A-44 | H | A-44 | H |
| 2254 | 0 | m-D11 | H | A-45 | H | A-45 | H |
| 2255 | 0 | m-D11 | H | A-46 | H | A-46 | H |
| 2256 | 0 | m-D11 | H | A-47 | H | A-47 | H |
| 2257 | 0 | m-D12 | H | A-1 | H | A-1 | H |
| 2258 | 0 | m-D12 | H | A-2 | H | A-2 | H |
| 2259 | 0 | m-D12 | H | A-3 | H | A-3 | H |
| 2260 | 0 | m-D12 | H | A-4 | H | A-4 | H |
| 2261 | 0 | m-D12 | H | A-5 | H | A-5 | H |
| 2262 | 0 | m-D12 | H | A-6 | H | A-6 | H |
| 2263 | 0 | m-D12 | H | A-7 | H | A-7 | H |
| 2264 | 0 | m-D12 | H | A-8 | H | A-8 | H |
| 2265 | 0 | m-D12 | H | A-9 | H | A-9 | H |
| 2266 | 0 | m-D12 | H | A-10 | H | A-10 | H |
| 2267 | 0 | m-D12 | H | A-11 | H | A-11 | H |
| 2268 | 0 | m-D12 | H | A-12 | H | A-12 | H |
| 2269 | 0 | m-D12 | H | A-13 | H | A-13 | H |
| 2270 | 0 | m-D12 | H | A-14 | H | A-14 | H |
| 2271 | 0 | m-D12 | H | A-15 | H | A-15 | H |
| 2272 | 0 | m-D12 | H | A-16 | H | A-16 | H |
| 2273 | 0 | m-D12 | H | A-17 | H | A-17 | H |
| 2274 | 0 | m-D12 | H | A-18 | H | A-18 | H |
| 2275 | 0 | m-D12 | H | A-19 | H | A-19 | H |
| 2276 | 0 | m-D12 | H | A-20 | H | A-20 | H |
| 2277 | 0 | m-D12 | H | A-21 | H | A-21 | H |
| 2278 | 0 | m-D12 | H | A-22 | H | A-22 | H |
| 2279 | 0 | m-D12 | H | A-23 | H | A-23 | H |
| 2280 | 0 | m-D12 | H | A-24 | H | A-24 | H |
| 2281 | 0 | m-D12 | H | A-25 | H | A-25 | H |
| 2282 | 0 | m-D12 | H | A-26 | H | A-26 | H |
| 2283 | 0 | m-D12 | H | A-27 | H | A-27 | H |
| 2284 | 0 | m-D12 | H | A-28 | H | A-28 | H |
| 2285 | 0 | m-D12 | H | A-29 | H | A-29 | H |
| 2286 | 0 | m-D12 | H | A-30 | H | A-30 | H |
| 2287 | 0 | m-D12 | H | A-31 | H | A-31 | H |
| 2288 | 0 | m-D12 | H | A-32 | H | A-32 | H |
| 2289 | 0 | m-D12 | H | A-33 | H | A-33 | H |
| 2290 | 0 | m-D12 | H | A-34 | H | A-34 | H |
| 2291 | 0 | m-D12 | H | A-35 | H | A-35 | H |
| 2292 | 0 | m-D12 | H | A-36 | H | A-36 | H |
| 2293 | 0 | m-D12 | H | A-37 | H | A-37 | H |
| 2294 | 0 | m-D12 | H | A-38 | H | A-38 | H |
| 2295 | 0 | m-D12 | H | A-39 | H | A-39 | H |
| 2296 | 0 | m-D12 | H | A-40 | H | A-40 | H |
| 2297 | 0 | m-D12 | H | A-41 | H | A-41 | H |
| 2298 | 0 | m-D12 | H | A-42 | H | A-42 | H |
| 2299 | 0 | m-D12 | H | A-43 | H | A-43 | H |
| 2300 | 0 | m-D12 | H | A-44 | H | A-44 | H |
| 2301 | 0 | m-D12 | H | A-45 | H | A-45 | H |
| 2302 | 0 | m-D12 | H | A-46 | H | A-46 | H |
| 2303 | 0 | m-D12 | H | A-47 | H | A-47 | H |
| 2304 | 0 | m-D13 | H | A-1 | H | A-1 | H |
| 2305 | 0 | m-D13 | H | A-2 | H | A-2 | H |
| 2306 | 0 | m-D13 | H | A-3 | H | A-3 | H |
| 2307 | 0 | m-D13 | H | A-4 | H | A-4 | H |
| 2308 | 0 | m-D13 | H | A-5 | H | A-5 | H |
| 2309 | 0 | m-D13 | H | A-6 | H | A-6 | H |
| 2310 | 0 | m-D13 | H | A-7 | H | A-7 | H |
| 2311 | 0 | m-D13 | H | A-8 | H | A-8 | H |
| 2312 | 0 | m-D13 | H | A-9 | H | A-9 | H |
| 2313 | 0 | m-D13 | H | A-10 | H | A-10 | H |
| 2314 | 0 | m-D13 | H | A-11 | H | A-11 | H |
| 2315 | 0 | m-D13 | H | A-12 | H | A-12 | H |
| 2316 | 0 | m-D13 | H | A-13 | H | A-13 | H |
| 2317 | 0 | m-D13 | H | A-14 | H | A-14 | H |
| 2318 | 0 | m-D13 | H | A-15 | H | A-15 | H |
| 2319 | 0 | m-D13 | H | A-16 | H | A-16 | H |
| 2320 | 0 | m-D13 | H | A-17 | H | A-17 | H |
| 2321 | 0 | m-D13 | H | A-18 | H | A-18 | H |
| 2322 | 0 | m-D13 | H | A-19 | H | A-19 | H |
| 2323 | 0 | m-D13 | H | A-20 | H | A-20 | H |
| 2324 | 0 | m-D13 | H | A-21 | H | A-21 | H |
| 2325 | 0 | m-D13 | H | A-22 | H | A-22 | H |
| 2326 | 0 | m-D13 | H | A-23 | H | A-23 | H |
| 2327 | 0 | m-D13 | H | A-24 | H | A-24 | H |
| 2328 | 0 | m-D13 | H | A-25 | H | A-25 | H |
| 2329 | 0 | m-D13 | H | A-26 | H | A-26 | H |
| 2330 | 0 | m-D13 | H | A-27 | H | A-27 | H |
| 2331 | 0 | m-D13 | H | A-28 | H | A-28 | H |
| 2332 | 0 | m-D13 | H | A-29 | H | A-29 | H |
| 2333 | 0 | m-D13 | H | A-30 | H | A-30 | H |
| 2334 | 0 | m-D13 | H | A-31 | H | A-31 | H |
| 2335 | 0 | m-D13 | H | A-32 | H | A-32 | H |
| 2336 | 0 | m-D13 | H | A-33 | H | A-33 | H |
| 2337 | 0 | m-D13 | H | A-34 | H | A-34 | H |
| 2338 | 0 | m-D13 | H | A-35 | H | A-35 | H |
| 2339 | 0 | m-D13 | H | A-36 | H | A-36 | H |
| 2340 | 0 | m-D13 | H | A-37 | H | A-37 | H |
| 2341 | 0 | m-D13 | H | A-38 | H | A-38 | H |
| 2342 | 0 | m-D13 | H | A-39 | H | A-39 | H |
| 2343 | 0 | m-D13 | H | A-40 | H | A-40 | H |
| 2344 | 0 | m-D13 | H | A-41 | H | A-41 | H |
| 2345 | 0 | m-D13 | H | A-42 | H | A-42 | H |
| 2346 | 0 | m-D13 | H | A-43 | H | A-43 | H |
| 2347 | 0 | m-D13 | H | A-44 | H | A-44 | H |
| 2348 | 0 | m-D13 | H | A-45 | H | A-45 | H |
| 2349 | 0 | m-D13 | H | A-46 | H | A-46 | H |
| 2350 | 0 | m-D13 | H | A-47 | H | A-47 | H |
| 2351 | 0 | m-D14 | H | A-1 | H | A-1 | H |
| 2352 | 0 | m-D14 | H | A-2 | H | A-2 | H |
| 2353 | 0 | m-D14 | H | A-3 | H | A-3 | H |
| 2354 | 0 | m-D14 | H | A-4 | H | A-4 | H |
| 2355 | 0 | m-D14 | H | A-5 | H | A-5 | H |
| 2356 | 0 | m-D14 | H | A-6 | H | A-6 | H |
| 2357 | 0 | m-D14 | H | A-7 | H | A-7 | H |
| 2358 | 0 | m-D14 | H | A-8 | H | A-8 | H |
| 2359 | 0 | m-D14 | H | A-9 | H | A-9 | H |
| 2360 | 0 | m-D14 | H | A-10 | H | A-10 | H |
| 2361 | 0 | m-D14 | H | A-11 | H | A-11 | H |
| 2362 | 0 | m-D14 | H | A-12 | H | A-12 | H |
| 2363 | 0 | m-D14 | H | A-13 | H | A-13 | H |
| 2364 | 0 | m-D14 | H | A-14 | H | A-14 | H |
| 2365 | 0 | m-D14 | H | A-15 | H | A-15 | H |
| 2366 | 0 | m-D14 | H | A-16 | H | A-16 | H |
| 2367 | 0 | m-D14 | H | A-17 | H | A-17 | H |
| 2368 | 0 | m-D14 | H | A-18 | H | A-18 | H |
| 2369 | 0 | m-D14 | H | A-19 | H | A-19 | H |
| 2370 | 0 | m-D14 | H | A-20 | H | A-20 | H |
| 2371 | 0 | m-D14 | H | A-21 | H | A-21 | H |
| 2372 | 0 | m-D14 | H | A-22 | H | A-22 | H |
| 2373 | 0 | m-D14 | H | A-23 | H | A-23 | H |
| 2374 | 0 | m-D14 | H | A-24 | H | A-24 | H |
| 2375 | 0 | m-D14 | H | A-25 | H | A-25 | H |
| 2376 | 0 | m-D14 | H | A-26 | H | A-26 | H |
| 2377 | 0 | m-D14 | H | A-27 | H | A-27 | H |
| 2378 | 0 | m-D14 | H | A-28 | H | A-28 | H |
| 2379 | 0 | m-D14 | H | A-29 | H | A-29 | H |
| 2380 | 0 | m-D14 | H | A-30 | H | A-30 | H |
| 2381 | 0 | m-D14 | H | A-31 | H | A-31 | H |
| 2382 | 0 | m-D14 | H | A-32 | H | A-32 | H |
| 2383 | 0 | m-D14 | H | A-33 | H | A-33 | H |
| 2384 | 0 | m-D14 | H | A-34 | H | A-34 | H |
| 2385 | 0 | m-D14 | H | A-35 | H | A-35 | H |
| 2386 | 0 | m-D14 | H | A-36 | H | A-36 | H |
| 2387 | 0 | m-D14 | H | A-37 | H | A-37 | H |
| 2388 | 0 | m-D14 | H | A-38 | H | A-38 | H |
| 2389 | 0 | m-D14 | H | A-39 | H | A-39 | H |
| 2390 | 0 | m-D14 | H | A-40 | H | A-40 | H |
| 2391 | 0 | m-D14 | H | A-41 | H | A-41 | H |
| 2392 | 0 | m-D14 | H | A-42 | H | A-42 | H |
| 2393 | 0 | m-D14 | H | A-43 | H | A-43 | H |
| 2394 | 0 | m-D14 | H | A-44 | H | A-44 | H |
| 2395 | 0 | m-D14 | H | A-45 | H | A-45 | H |
| 2396 | 0 | m-D14 | H | A-46 | H | A-46 | H |
| 2397 | 0 | m-D14 | H | A-47 | H | A-47 | H |
| 2398 | 0 | m-D1 | H | A-1 | H | m-D1 | H |
| 2399 | 0 | m-D1 | H | A-2 | H | m-D1 | H |
| 2400 | 0 | m-D1 | H | A-3 | H | m-D1 | H |
| 2401 | 0 | m-D1 | H | A-4 | H | m-D1 | H |
| 2402 | 0 | m-D1 | H | A-5 | H | m-D1 | H |
| 2403 | 0 | m-D1 | H | A-6 | H | m-D1 | H |
| 2404 | 0 | m-D1 | H | A-7 | H | m-D1 | H |
| 2405 | 0 | m-D1 | H | A-8 | H | m-D1 | H |
| 2406 | 0 | m-D1 | H | A-9 | H | m-D1 | H |
| 2407 | 0 | m-D1 | H | A-10 | H | m-D1 | H |
| 2408 | 0 | m-D1 | H | A-11 | H | m-D1 | H |
| 2409 | 0 | m-D1 | H | A-12 | H | m-D1 | H |
| 2410 | 0 | m-D1 | H | A-13 | H | m-D1 | H |
| 2411 | 0 | m-D1 | H | A-14 | H | m-D1 | H |
| 2412 | 0 | m-D1 | H | A-15 | H | m-D1 | H |
| 2413 | 0 | m-D1 | H | A-16 | H | m-D1 | H |
| 2414 | 0 | m-D1 | H | A-17 | H | m-D1 | H |
| 2415 | 0 | m-D1 | H | A-18 | H | m-D1 | H |
| 2416 | 0 | m-D1 | H | A-19 | H | m-D1 | H |
| 2417 | 0 | m-D1 | H | A-20 | H | m-D1 | H |
| 2418 | 0 | m-D1 | H | A-21 | H | m-D1 | H |
| 2419 | 0 | m-D1 | H | A-22 | H | m-D1 | H |
| 2420 | 0 | m-D1 | H | A-23 | H | m-D1 | H |
| 2421 | 0 | m-D1 | H | A-24 | H | m-D1 | H |
| 2422 | 0 | m-D1 | H | A-25 | H | m-D1 | H |
| 2423 | 0 | m-D1 | H | A-26 | H | m-D1 | H |
| 2424 | 0 | m-D1 | H | A-27 | H | m-D1 | H |
| 2425 | 0 | m-D1 | H | A-28 | H | m-D1 | H |
| 2426 | 0 | m-D1 | H | A-29 | H | m-D1 | H |
| 2427 | 0 | m-D1 | H | A-30 | H | m-D1 | H |
| 2428 | 0 | m-D1 | H | A-31 | H | m-D1 | H |
| 2429 | 0 | m-D1 | H | A-32 | H | m-D1 | H |
| 2430 | 0 | m-D1 | H | A-33 | H | m-D1 | H |
| 2431 | 0 | m-D1 | H | A-34 | H | m-D1 | H |
| 2432 | 0 | m-D1 | H | A-35 | H | m-D1 | H |
| 2433 | 0 | m-D1 | H | A-36 | H | m-D1 | H |
| 2434 | 0 | m-D1 | H | A-37 | H | m-D1 | H |
| 2435 | 0 | m-D1 | H | A-38 | H | m-D1 | H |
| 2436 | 0 | m-D1 | H | A-39 | H | m-D1 | H |
| 2437 | 0 | m-D1 | H | A-40 | H | m-D1 | H |
| 2438 | 0 | m-D1 | H | A-41 | H | m-D1 | H |
| 2439 | 0 | m-D1 | H | A-42 | H | m-D1 | H |
| 2440 | 0 | m-D1 | H | A-43 | H | m-D1 | H |
| 2441 | 0 | m-D1 | H | A-44 | H | m-D1 | H |
| 2442 | 0 | m-D1 | H | A-45 | H | m-D1 | H |
| 2443 | 0 | m-D1 | H | A-46 | H | m-D1 | H |
| 2444 | 0 | m-D1 | H | A-47 | H | m-D1 | H |
| 2445 | 0 | m-D2 | H | A-1 | H | m-D2 | H |
| 2446 | 0 | m-D2 | H | A-2 | H | m-D2 | H |
| 2447 | 0 | m-D2 | H | A-3 | H | m-D2 | H |
| 2448 | 0 | m-D2 | H | A-4 | H | m-D2 | H |
| 2449 | 0 | m-D2 | H | A-5 | H | m-D2 | H |
| 2450 | 0 | m-D2 | H | A-6 | H | m-D2 | H |
| 2451 | 0 | m-D2 | H | A-7 | H | m-D2 | H |
| 2452 | 0 | m-D2 | H | A-8 | H | m-D2 | H |
| 2453 | 0 | m-D2 | H | A-9 | H | m-D2 | H |
| 2454 | 0 | m-D2 | H | A-10 | H | m-D2 | H |
| 2455 | 0 | m-D2 | H | A-11 | H | m-D2 | H |
| 2456 | 0 | m-D2 | H | A-12 | H | m-D2 | H |
| 2457 | 0 | m-D2 | H | A-13 | H | m-D2 | H |
| 2458 | 0 | m-D2 | H | A-14 | H | m-D2 | H |
| 2459 | 0 | m-D2 | H | A-15 | H | m-D2 | H |
| 2460 | 0 | m-D2 | H | A-16 | H | m-D2 | H |
| 2461 | 0 | m-D2 | H | A-17 | H | m-D2 | H |
| 2462 | 0 | m-D2 | H | A-18 | H | m-D2 | H |
| 2463 | 0 | m-D2 | H | A-19 | H | m-D2 | H |
| 2464 | 0 | m-D2 | H | A-20 | H | m-D2 | H |
| 2465 | 0 | m-D2 | H | A-21 | H | m-D2 | H |
| 2466 | 0 | m-D2 | H | A-22 | H | m-D2 | H |
| 2467 | 0 | m-D2 | H | A-23 | H | m-D2 | H |
| 2468 | 0 | m-D2 | H | A-24 | H | m-D2 | H |
| 2469 | 0 | m-D2 | H | A-25 | H | m-D2 | H |
| 2470 | 0 | m-D2 | H | A-26 | H | m-D2 | H |
| 2471 | 0 | m-D2 | H | A-27 | H | m-D2 | H |
| 2472 | 0 | m-D2 | H | A-28 | H | m-D2 | H |
| 2473 | 0 | m-D2 | H | A-29 | H | m-D2 | H |
| 2474 | 0 | m-D2 | H | A-30 | H | m-D2 | H |
| 2475 | 0 | m-D2 | H | A-31 | H | m-D2 | H |
| 2476 | 0 | m-D2 | H | A-32 | H | m-D2 | H |
| 2477 | 0 | m-D2 | H | A-33 | H | m-D2 | H |
| 2478 | 0 | m-D2 | H | A-34 | H | m-D2 | H |
| 2479 | 0 | m-D2 | H | A-35 | H | m-D2 | H |
| 2480 | 0 | m-D2 | H | A-36 | H | m-D2 | H |
| 2481 | 0 | m-D2 | H | A-37 | H | m-D2 | H |
| 2482 | 0 | m-D2 | H | A-38 | H | m-D2 | H |
| 2483 | 0 | m-D2 | H | A-39 | H | m-D2 | H |
| 2484 | 0 | m-D2 | H | A-40 | H | m-D2 | H |
| 2485 | 0 | m-D2 | H | A-41 | H | m-D2 | H |
| 2486 | 0 | m-D2 | H | A-42 | H | m-D2 | H |
| 2487 | 0 | m-D2 | H | A-43 | H | m-D2 | H |
| 2488 | 0 | m-D2 | H | A-44 | H | m-D2 | H |
| 2489 | 0 | m-D2 | H | A-45 | H | m-D2 | H |
| 2490 | 0 | m-D2 | H | A-46 | H | m-D2 | H |
| 2491 | 0 | m-D2 | H | A-47 | H | m-D2 | H |
| 2492 | 0 | m-D3 | H | A-1 | H | m-D3 | H |
| 2493 | 0 | m-D3 | H | A-2 | H | m-D3 | H |
| 2494 | 0 | m-D3 | H | A-3 | H | m-D3 | H |
| 2495 | 0 | m-D3 | H | A-4 | H | m-D3 | H |
| 2496 | 0 | m-D3 | H | A-5 | H | m-D3 | H |
| 2497 | 0 | m-D3 | H | A-6 | H | m-D3 | H |
| 2498 | 0 | m-D3 | H | A-7 | H | m-D3 | H |
| 2499 | 0 | m-D3 | H | A-8 | H | m-D3 | H |
| 2500 | 0 | m-D3 | H | A-9 | H | m-D3 | H |
| 2501 | 0 | m-D3 | H | A-10 | H | m-D3 | H |
| 2502 | 0 | m-D3 | H | A-11 | H | m-D3 | H |
| 2503 | 0 | m-D3 | H | A-12 | H | m-D3 | H |
| 2504 | 0 | m-D3 | H | A-13 | H | m-D3 | H |
| 2505 | 0 | m-D3 | H | A-14 | H | m-D3 | H |
| 2506 | 0 | m-D3 | H | A-15 | H | m-D3 | H |
| 2507 | 0 | m-D3 | H | A-16 | H | m-D3 | H |
| 2508 | 0 | m-D3 | H | A-17 | H | m-D3 | H |
| 2509 | 0 | m-D3 | H | A-18 | H | m-D3 | H |
| 2510 | 0 | m-D3 | H | A-19 | H | m-D3 | H |
| 2511 | 0 | m-D3 | H | A-20 | H | m-D3 | H |
| 2512 | 0 | m-D3 | H | A-21 | H | m-D3 | H |
| 2513 | 0 | m-D3 | H | A-22 | H | m-D3 | H |
| 2514 | 0 | m-D3 | H | A-23 | H | m-D3 | H |
| 2515 | 0 | m-D3 | H | A-24 | H | m-D3 | H |
| 2516 | 0 | m-D3 | H | A-25 | H | m-D3 | H |
| 2517 | 0 | m-D3 | H | A-26 | H | m-D3 | H |
| 2518 | 0 | m-D3 | H | A-27 | H | m-D3 | H |
| 2519 | 0 | m-D3 | H | A-28 | H | m-D3 | H |
| 2520 | 0 | m-D3 | H | A-29 | H | m-D3 | H |
| 2521 | 0 | m-D3 | H | A-30 | H | m-D3 | H |
| 2522 | 0 | m-D3 | H | A-31 | H | m-D3 | H |
| 2523 | 0 | m-D3 | H | A-32 | H | m-D3 | H |
| 2524 | 0 | m-D3 | H | A-33 | H | m-D3 | H |
| 2525 | 0 | m-D3 | H | A-34 | H | m-D3 | H |
| 2526 | 0 | m-D3 | H | A-35 | H | m-D3 | H |
| 2527 | 0 | m-D3 | H | A-36 | H | m-D3 | H |
| 2528 | 0 | m-D3 | H | A-37 | H | m-D3 | H |
| 2529 | 0 | m-D3 | H | A-38 | H | m-D3 | H |
| 2530 | 0 | m-D3 | H | A-39 | H | m-D3 | H |
| 2531 | 0 | m-D3 | H | A-40 | H | m-D3 | H |
| 2532 | 0 | m-D3 | H | A-41 | H | m-D3 | H |
| 2533 | 0 | m-D3 | H | A-42 | H | m-D3 | H |
| 2534 | 0 | m-D3 | H | A-43 | H | m-D3 | H |
| 2535 | 0 | m-D3 | H | A-44 | H | m-D3 | H |
| 2536 | 0 | m-D3 | H | A-45 | H | m-D3 | H |
| 2537 | 0 | m-D3 | H | A-46 | H | m-D3 | H |
| 2538 | 0 | m-D3 | H | A-47 | H | m-D3 | H |
| 2539 | 0 | m-D5 | H | A-1 | H | m-D5 | H |
| 2540 | 0 | m-D5 | H | A-2 | H | m-D5 | H |
| 2541 | 0 | m-D5 | H | A-3 | H | m-D5 | H |
| 2542 | 0 | m-D5 | H | A-4 | H | m-D5 | H |
| 2543 | 0 | m-D5 | H | A-5 | H | m-D5 | H |
| 2544 | 0 | m-D5 | H | A-6 | H | m-D5 | H |
| 2545 | 0 | m-D5 | H | A-7 | H | m-D5 | H |
| 2546 | 0 | m-D5 | H | A-8 | H | m-D5 | H |
| 2547 | 0 | m-D5 | H | A-9 | H | m-D5 | H |
| 2548 | 0 | m-D5 | H | A-10 | H | m-D5 | H |
| 2549 | 0 | m-D5 | H | A-11 | H | m-D5 | H |
| 2550 | 0 | m-D5 | H | A-12 | H | m-D5 | H |
| 2551 | 0 | m-D5 | H | A-13 | H | m-D5 | H |
| 2552 | 0 | m-D5 | H | A-14 | H | m-D5 | H |
| 2553 | 0 | m-D5 | H | A-15 | H | m-D5 | H |
| 2554 | 0 | m-D5 | H | A-16 | H | m-D5 | H |
| 2555 | 0 | m-D5 | H | A-17 | H | m-D5 | H |
| 2556 | 0 | m-D5 | H | A-18 | H | m-D5 | H |
| 2557 | 0 | m-D5 | H | A-19 | H | m-D5 | H |
| 2558 | 0 | m-D5 | H | A-20 | H | m-D5 | H |
| 2559 | 0 | m-D5 | H | A-21 | H | m-D5 | H |
| 2560 | 0 | m-D5 | H | A-22 | H | m-D5 | H |
| 2561 | 0 | m-D5 | H | A-23 | H | m-D5 | H |
| 2562 | 0 | m-D5 | H | A-24 | H | m-D5 | H |
| 2563 | 0 | m-D5 | H | A-25 | H | m-D5 | H |
| 2564 | 0 | m-D5 | H | A-26 | H | m-D5 | H |
| 2565 | 0 | m-D5 | H | A-27 | H | m-D5 | H |
| 2566 | 0 | m-D5 | H | A-28 | H | m-D5 | H |
| 2567 | 0 | m-D5 | H | A-29 | H | m-D5 | H |
| 2568 | 0 | m-D5 | H | A-30 | H | m-D5 | H |
| 2569 | 0 | m-D5 | H | A-31 | H | m-D5 | H |
| 2570 | 0 | m-D5 | H | A-32 | H | m-D5 | H |
| 2571 | 0 | m-D5 | H | A-33 | H | m-D5 | H |
| 2572 | 0 | m-D5 | H | A-34 | H | m-D5 | H |
| 2573 | 0 | m-D5 | H | A-35 | H | m-D5 | H |
| 2574 | 0 | m-D5 | H | A-36 | H | m-D5 | H |
| 2575 | 0 | m-D5 | H | A-37 | H | m-D5 | H |
| 2576 | 0 | m-D5 | H | A-38 | H | m-D5 | H |
| 2577 | 0 | m-D5 | H | A-39 | H | m-D5 | H |
| 2578 | 0 | m-D5 | H | A-40 | H | m-D5 | H |
| 2579 | 0 | m-D5 | H | A-41 | H | m-D5 | H |
| 2580 | 0 | m-D5 | H | A-42 | H | m-D5 | H |
| 2581 | 0 | m-D5 | H | A-43 | H | m-D5 | H |
| 2582 | 0 | m-D5 | H | A-44 | H | m-D5 | H |
| 2583 | 0 | m-D5 | H | A-45 | H | m-D5 | H |
| 2584 | 0 | m-D5 | H | A-46 | H | m-D5 | H |
| 2585 | 0 | m-D5 | H | A-47 | H | m-D5 | H |
| 2586 | 0 | m-D9 | H | A-1 | H | m-D9 | H |
| 2587 | 0 | m-D9 | H | A-2 | H | m-D9 | H |
| 2588 | 0 | m-D9 | H | A-3 | H | m-D9 | H |
| 2589 | 0 | m-D9 | H | A-4 | H | m-D9 | H |
| 2590 | 0 | m-D9 | H | A-5 | H | m-D9 | H |
| 2591 | 0 | m-D9 | H | A-6 | H | m-D9 | H |
| 2592 | 0 | m-D9 | H | A-7 | H | m-D9 | H |
| 2593 | 0 | m-D9 | H | A-8 | H | m-D9 | H |
| 2594 | 0 | m-D9 | H | A-9 | H | m-D9 | H |
| 2595 | 0 | m-D9 | H | A-10 | H | m-D9 | H |
| 2596 | 0 | m-D9 | H | A-11 | H | m-D9 | H |
| 2597 | 0 | m-D9 | H | A-12 | H | m-D9 | H |
| 2598 | 0 | m-D9 | H | A-13 | H | m-D9 | H |
| 2599 | 0 | m-D9 | H | A-14 | H | m-D9 | H |
| 2600 | 0 | m-D9 | H | A-15 | H | m-D9 | H |
| 2601 | 0 | m-D9 | H | A-16 | H | m-D9 | H |
| 2602 | 0 | m-D9 | H | A-17 | H | m-D9 | H |
| 2603 | 0 | m-D9 | H | A-18 | H | m-D9 | H |
| 2604 | 0 | m-D9 | H | A-19 | H | m-D9 | H |
| 2605 | 0 | m-D9 | H | A-20 | H | m-D9 | H |
| 2606 | 0 | m-D9 | H | A-21 | H | m-D9 | H |
| 2607 | 0 | m-D9 | H | A-22 | H | m-D9 | H |
| 2608 | 0 | m-D9 | H | A-23 | H | m-D9 | H |
| 2609 | 0 | m-D9 | H | A-24 | H | m-D9 | H |
| 2610 | 0 | m-D9 | H | A-25 | H | m-D9 | H |
| 2611 | 0 | m-D9 | H | A-26 | H | m-D9 | H |
| 2612 | 0 | m-D9 | H | A-27 | H | m-D9 | H |
| 2613 | 0 | m-D9 | H | A-28 | H | m-D9 | H |
| 2614 | 0 | m-D9 | H | A-29 | H | m-D9 | H |
| 2615 | 0 | m-D9 | H | A-30 | H | m-D9 | H |
| 2616 | 0 | m-D9 | H | A-31 | H | m-D9 | H |
| 2617 | 0 | m-D9 | H | A-32 | H | m-D9 | H |
| 2618 | 0 | m-D9 | H | A-33 | H | m-D9 | H |
| 2619 | 0 | m-D9 | H | A-34 | H | m-D9 | H |
| 2620 | 0 | m-D9 | H | A-35 | H | m-D9 | H |
| 2621 | 0 | m-D9 | H | A-36 | H | m-D9 | H |
| 2622 | 0 | m-D9 | H | A-37 | H | m-D9 | H |
| 2623 | 0 | m-D9 | H | A-38 | H | m-D9 | H |
| 2624 | 0 | m-D9 | H | A-39 | H | m-D9 | H |
| 2625 | 0 | m-D9 | H | A-40 | H | m-D9 | H |
| 2626 | 0 | m-D9 | H | A-41 | H | m-D9 | H |
| 2627 | 0 | m-D9 | H | A-42 | H | m-D9 | H |
| 2628 | 0 | m-D9 | H | A-43 | H | m-D9 | H |
| 2629 | 0 | m-D9 | H | A-44 | H | m-D9 | H |
| 2630 | 0 | m-D9 | H | A-45 | H | m-D9 | H |
| 2631 | 0 | m-D9 | H | A-46 | H | m-D9 | H |
| 2632 | 0 | m-D9 | H | A-47 | H | m-D9 | H |
| 2633 | 0 | m-D11 | H | A-1 | H | m-D11 | H |
| 2634 | 0 | m-D11 | H | A-2 | H | m-D11 | H |
| 2635 | 0 | m-D11 | H | A-3 | H | m-D11 | H |
| 2636 | 0 | m-D11 | H | A-4 | H | m-D11 | H |
| 2637 | 0 | m-D11 | H | A-5 | H | m-D11 | H |
| 2638 | 0 | m-D11 | H | A-6 | H | m-D11 | H |
| 2639 | 0 | m-D11 | H | A-7 | H | m-D11 | H |
| 2640 | 0 | m-D11 | H | A-8 | H | m-D11 | H |
| 2641 | 0 | m-D11 | H | A-9 | H | m-D11 | H |
| 2642 | 0 | m-D11 | H | A-10 | H | m-D11 | H |
| 2643 | 0 | m-D11 | H | A-11 | H | m-D11 | H |
| 2644 | 0 | m-D11 | H | A-12 | H | m-D11 | H |
| 2645 | 0 | m-D11 | H | A-13 | H | m-D11 | H |
| 2646 | 0 | m-D11 | H | A-14 | H | m-D11 | H |
| 2647 | 0 | m-D11 | H | A-15 | H | m-D11 | H |
| 2648 | 0 | m-D11 | H | A-16 | H | m-D11 | H |
| 2649 | 0 | m-D11 | H | A-17 | H | m-D11 | H |
| 2650 | 0 | m-D11 | H | A-18 | H | m-D11 | H |
| 2651 | 0 | m-D11 | H | A-19 | H | m-D11 | H |
| 2652 | 0 | m-D11 | H | A-20 | H | m-D11 | H |
| 2653 | 0 | m-D11 | H | A-21 | H | m-D11 | H |
| 2654 | 0 | m-D11 | H | A-22 | H | m-D11 | H |
| 2655 | 0 | m-D11 | H | A-23 | H | m-D11 | H |
| 2656 | 0 | m-D11 | H | A-24 | H | m-D11 | H |
| 2657 | 0 | m-D11 | H | A-25 | H | m-D11 | H |
| 2658 | 0 | m-D11 | H | A-26 | H | m-D11 | H |
| 2659 | 0 | m-D11 | H | A-27 | H | m-D11 | H |
| 2660 | 0 | m-D11 | H | A-28 | H | m-D11 | H |
| 2661 | 0 | m-D11 | H | A-29 | H | m-D11 | H |
| 2662 | 0 | m-D11 | H | A-30 | H | m-D11 | H |
| 2663 | 0 | m-D11 | H | A-31 | H | m-D11 | H |
| 2664 | 0 | m-D11 | H | A-32 | H | m-D11 | H |
| 2665 | 0 | m-D11 | H | A-33 | H | m-D11 | H |
| 2666 | 0 | m-D11 | H | A-34 | H | m-D11 | H |
| 2667 | 0 | m-D11 | H | A-35 | H | m-D11 | H |
| 2668 | 0 | m-D11 | H | A-36 | H | m-D11 | H |
| 2669 | 0 | m-D11 | H | A-37 | H | m-D11 | H |
| 2670 | 0 | m-D11 | H | A-38 | H | m-D11 | H |
| 2671 | 0 | m-D11 | H | A-39 | H | m-D11 | H |
| 2672 | 0 | m-D11 | H | A-40 | H | m-D11 | H |
| 2673 | 0 | m-D11 | H | A-41 | H | m-D11 | H |
| 2674 | 0 | m-D11 | H | A-42 | H | m-D11 | H |
| 2675 | 0 | m-D11 | H | A-43 | H | m-D11 | H |
| 2676 | 0 | m-D11 | H | A-44 | H | m-D11 | H |
| 2677 | 0 | m-D11 | H | A-45 | H | m-D11 | H |
| 2678 | 0 | m-D11 | H | A-46 | H | m-D11 | H |
| 2679 | 0 | m-D11 | H | A-47 | H | m-D11 | H |
| 2680 | 0 | m-D12 | H | A-1 | H | m-D12 | H |
| 2681 | 0 | m-D12 | H | A-2 | H | m-D12 | H |
| 2682 | 0 | m-D12 | H | A-3 | H | m-D12 | H |
| 2683 | 0 | m-D12 | H | A-4 | H | m-D12 | H |
| 2684 | 0 | m-D12 | H | A-5 | H | m-D12 | H |
| 2685 | 0 | m-D12 | H | A-6 | H | m-D12 | H |
| 2686 | 0 | m-D12 | H | A-7 | H | m-D12 | H |
| 2687 | 0 | m-D12 | H | A-8 | H | m-D12 | H |
| 2688 | 0 | m-D12 | H | A-9 | H | m-D12 | H |
| 2689 | 0 | m-D12 | H | A-10 | H | m-D12 | H |
| 2690 | 0 | m-D12 | H | A-11 | H | m-D12 | H |
| 2691 | 0 | m-D12 | H | A-12 | H | m-D12 | H |
| 2692 | 0 | m-D12 | H | A-13 | H | m-D12 | H |
| 2693 | 0 | m-D12 | H | A-14 | H | m-D12 | H |
| 2694 | 0 | m-D12 | H | A-15 | H | m-D12 | H |
| 2695 | 0 | m-D12 | H | A-16 | H | m-D12 | H |
| 2696 | 0 | m-D12 | H | A-17 | H | m-D12 | H |
| 2697 | 0 | m-D12 | H | A-18 | H | m-D12 | H |
| 2698 | 0 | m-D12 | H | A-19 | H | m-D12 | H |
| 2699 | 0 | m-D12 | H | A-20 | H | m-D12 | H |
| 2700 | 0 | m-D12 | H | A-21 | H | m-D12 | H |
| 2701 | 0 | m-D12 | H | A-22 | H | m-D12 | H |
| 2702 | 0 | m-D12 | H | A-23 | H | m-D12 | H |
| 2703 | 0 | m-D12 | H | A-24 | H | m-D12 | H |
| 2704 | 0 | m-D12 | H | A-25 | H | m-D12 | H |
| 2705 | 0 | m-D12 | H | A-26 | H | m-D12 | H |
| 2706 | 0 | m-D12 | H | A-27 | H | m-D12 | H |
| 2707 | 0 | m-D12 | H | A-28 | H | m-D12 | H |
| 2708 | 0 | m-D12 | H | A-29 | H | m-D12 | H |
| 2709 | 0 | m-D12 | H | A-30 | H | m-D12 | H |
| 2710 | 0 | m-D12 | H | A-31 | H | m-D12 | H |
| 2711 | 0 | m-D12 | H | A-32 | H | m-D12 | H |
| 2712 | 0 | m-D12 | H | A-33 | H | m-D12 | H |
| 2713 | 0 | m-D12 | H | A-34 | H | m-D12 | H |
| 2714 | 0 | m-D12 | H | A-35 | H | m-D12 | H |
| 2715 | 0 | m-D12 | H | A-36 | H | m-D12 | H |
| 2716 | 0 | m-D12 | H | A-37 | H | m-D12 | H |
| 2717 | 0 | m-D12 | H | A-38 | H | m-D12 | H |
| 2718 | 0 | m-D12 | H | A-39 | H | m-D12 | H |
| 2719 | 0 | m-D12 | H | A-40 | H | m-D12 | H |
| 2720 | 0 | m-D12 | H | A-41 | H | m-D12 | H |
| 2721 | 0 | m-D12 | H | A-42 | H | m-D12 | H |
| 2722 | 0 | m-D12 | H | A-43 | H | m-D12 | H |
| 2723 | 0 | m-D12 | H | A-44 | H | m-D12 | H |
| 2724 | 0 | m-D12 | H | A-45 | H | m-D12 | H |
| 2725 | 0 | m-D12 | H | A-46 | H | m-D12 | H |
| 2726 | 0 | m-D12 | H | A-47 | H | m-D12 | H |
| 2727 | 0 | m-D13 | H | A-1 | H | m-D13 | H |
| 2728 | 0 | m-D13 | H | A-2 | H | m-D13 | H |
| 2729 | 0 | m-D13 | H | A-3 | H | m-D13 | H |
| 2730 | 0 | m-D13 | H | A-4 | H | m-D13 | H |
| 2731 | 0 | m-D13 | H | A-5 | H | m-D13 | H |
| 2732 | 0 | m-D13 | H | A-6 | H | m-D13 | H |
| 2733 | 0 | m-D13 | H | A-7 | H | m-D13 | H |
| 2734 | 0 | m-D13 | H | A-8 | H | m-D13 | H |
| 2735 | 0 | m-D13 | H | A-9 | H | m-D13 | H |
| 2736 | 0 | m-D13 | H | A-10 | H | m-D13 | H |
| 2737 | 0 | m-D13 | H | A-11 | H | m-D13 | H |
| 2738 | 0 | m-D13 | H | A-12 | H | m-D13 | H |
| 2739 | 0 | m-D13 | H | A-13 | H | m-D13 | H |
| 2740 | 0 | m-D13 | H | A-14 | H | m-D13 | H |
| 2741 | 0 | m-D13 | H | A-15 | H | m-D13 | H |
| 2742 | 0 | m-D13 | H | A-16 | H | m-D13 | H |
| 2743 | 0 | m-D13 | H | A-17 | H | m-D13 | H |
| 2744 | 0 | m-D13 | H | A-18 | H | m-D13 | H |
| 2745 | 0 | m-D13 | H | A-19 | H | m-D13 | H |
| 2746 | 0 | m-D13 | H | A-20 | H | m-D13 | H |
| 2747 | 0 | m-D13 | H | A-21 | H | m-D13 | H |
| 2748 | 0 | m-D13 | H | A-22 | H | m-D13 | H |
| 2749 | 0 | m-D13 | H | A-23 | H | m-D13 | H |
| 2750 | 0 | m-D13 | H | A-24 | H | m-D13 | H |
| 2751 | 0 | m-D13 | H | A-25 | H | m-D13 | H |
| 2752 | 0 | m-D13 | H | A-26 | H | m-D13 | H |
| 2753 | 0 | m-D13 | H | A-27 | H | m-D13 | H |
| 2754 | 0 | m-D13 | H | A-28 | H | m-D13 | H |
| 2755 | 0 | m-D13 | H | A-29 | H | m-D13 | H |
| 2756 | 0 | m-D13 | H | A-30 | H | m-D13 | H |
| 2757 | 0 | m-D13 | H | A-31 | H | m-D13 | H |
| 2758 | 0 | m-D13 | H | A-32 | H | m-D13 | H |
| 2759 | 0 | m-D13 | H | A-33 | H | m-D13 | H |
| 2760 | 0 | m-D13 | H | A-34 | H | m-D13 | H |
| 2761 | 0 | m-D13 | H | A-35 | H | m-D13 | H |
| 2762 | 0 | m-D13 | H | A-36 | H | m-D13 | H |
| 2763 | 0 | m-D13 | H | A-37 | H | m-D13 | H |
| 2764 | 0 | m-D13 | H | A-38 | H | m-D13 | H |
| 2765 | 0 | m-D13 | H | A-39 | H | m-D13 | H |
| 2766 | 0 | m-D13 | H | A-40 | H | m-D13 | H |
| 2767 | 0 | m-D13 | H | A-41 | H | m-D13 | H |
| 2768 | 0 | m-D13 | H | A-42 | H | m-D13 | H |
| 2769 | 0 | m-D13 | H | A-43 | H | m-D13 | H |
| 2770 | 0 | m-D13 | H | A-44 | H | m-D13 | H |
| 2771 | 0 | m-D13 | H | A-45 | H | m-D13 | H |
| 2772 | 0 | m-D13 | H | A-46 | H | m-D13 | H |
| 2773 | 0 | m-D13 | H | A-47 | H | m-D13 | H |
| 2774 | 0 | m-D14 | H | A-1 | H | m-D14 | H |
| 2775 | 0 | m-D14 | H | A-2 | H | m-D14 | H |
| 2776 | 0 | m-D14 | H | A-3 | H | m-D14 | H |
| 2777 | 0 | m-D14 | H | A-4 | H | m-D14 | H |
| 2778 | 0 | m-D14 | H | A-5 | H | m-D14 | H |
| 2779 | 0 | m-D14 | H | A-6 | H | m-D14 | H |
| 2780 | 0 | m-D14 | H | A-7 | H | m-D14 | H |
| 2781 | 0 | m-D14 | H | A-8 | H | m-D14 | H |
| 2782 | 0 | m-D14 | H | A-9 | H | m-D14 | H |
| 2783 | 0 | m-D14 | H | A-10 | H | m-D14 | H |
| 2784 | 0 | m-D14 | H | A-11 | H | m-D14 | H |
| 2785 | 0 | m-D14 | H | A-12 | H | m-D14 | H |
| 2786 | 0 | m-D14 | H | A-13 | H | m-D14 | H |
| 2787 | 0 | m-D14 | H | A-14 | H | m-D14 | H |
| 2788 | 0 | m-D14 | H | A-15 | H | m-D14 | H |
| 2789 | 0 | m-D14 | H | A-16 | H | m-D14 | H |
| 2790 | 0 | m-D14 | H | A-17 | H | m-D14 | H |
| 2791 | 0 | m-D14 | H | A-18 | H | m-D14 | H |
| 2792 | 0 | m-D14 | H | A-19 | H | m-D14 | H |
| 2793 | 0 | m-D14 | H | A-20 | H | m-D14 | H |
| 2794 | 0 | m-D14 | H | A-21 | H | m-D14 | H |
| 2795 | 0 | m-D14 | H | A-22 | H | m-D14 | H |
| 2796 | 0 | m-D14 | H | A-23 | H | m-D14 | H |
| 2797 | 0 | m-D14 | H | A-24 | H | m-D14 | H |
| 2798 | 0 | m-D14 | H | A-25 | H | m-D14 | H |
| 2799 | 0 | m-D14 | H | A-26 | H | m-D14 | H |
| 2800 | 0 | m-D14 | H | A-27 | H | m-D14 | H |
| 2801 | 0 | m-D14 | H | A-28 | H | m-D14 | H |
| 2802 | 0 | m-D14 | H | A-29 | H | m-D14 | H |
| 2803 | 0 | m-D14 | H | A-30 | H | m-D14 | H |
| 2804 | 0 | m-D14 | H | A-31 | H | m-D14 | H |
| 2805 | 0 | m-D14 | H | A-32 | H | m-D14 | H |
| 2806 | 0 | m-D14 | H | A-33 | H | m-D14 | H |
| 2807 | 0 | m-D14 | H | A-34 | H | m-D14 | H |
| 2808 | 0 | m-D14 | H | A-35 | H | m-D14 | H |
| 2809 | 0 | m-D14 | H | A-36 | H | m-D14 | H |
| 2810 | 0 | m-D14 | H | A-37 | H | m-D14 | H |
| 2811 | 0 | m-D14 | H | A-38 | H | m-D14 | H |
| 2812 | 0 | m-D14 | H | A-39 | H | m-D14 | H |
| 2813 | 0 | m-D14 | H | A-40 | H | m-D14 | H |
| 2814 | 0 | m-D14 | H | A-41 | H | m-D14 | H |
| 2815 | 0 | m-D14 | H | A-42 | H | m-D14 | H |
| 2816 | 0 | m-D14 | H | A-43 | H | m-D14 | H |
| 2817 | 0 | m-D14 | H | A-44 | H | m-D14 | H |
| 2818 | 0 | m-D14 | H | A-45 | H | m-D14 | H |
| 2819 | 0 | m-D14 | H | A-46 | H | m-D14 | H |
| 2820 | 0 | m-D14 | H | A-47 | H | m-D14 | H |
| 2821 | 0 | m-D1 | H | A-1 | A-1 | H | H |
| 2822 | 0 | m-D1 | H | A-2 | A-2 | H | H |
| 2823 | 0 | m-D1 | H | A-3 | A-3 | H | H |
| 2824 | 0 | m-D1 | H | A-4 | A-4 | H | H |
| 2825 | 0 | m-D1 | H | A-5 | A-5 | H | H |
| 2826 | 0 | m-D1 | H | A-6 | A-6 | H | H |
| 2827 | 0 | m-D1 | H | A-7 | A-7 | H | H |
| 2828 | 0 | m-D1 | H | A-8 | A-8 | H | H |
| 2829 | 0 | m-D1 | H | A-9 | A-9 | H | H |
| 2830 | 0 | m-D1 | H | A-10 | A-10 | H | H |
| 2831 | 0 | m-D1 | H | A-11 | A-11 | H | H |
| 2832 | 0 | m-D1 | H | A-12 | A-12 | H | H |
| 2833 | 0 | m-D1 | H | A-13 | A-13 | H | H |
| 2834 | 0 | m-D1 | H | A-14 | A-14 | H | H |
| 2835 | 0 | m-D1 | H | A-15 | A-15 | H | H |
| 2836 | 0 | m-D1 | H | A-16 | A-16 | H | H |
| 2837 | 0 | m-D1 | H | A-17 | A-17 | H | H |
| 2838 | 0 | m-D1 | H | A-18 | A-18 | H | H |
| 2839 | 0 | m-D1 | H | A-19 | A-19 | H | H |
| 2840 | 0 | m-D1 | H | A-20 | A-20 | H | H |
| 2841 | 0 | m-D1 | H | A-21 | A-21 | H | H |
| 2842 | 0 | m-D1 | H | A-22 | A-22 | H | H |
| 2843 | 0 | m-D1 | H | A-23 | A-23 | H | H |
| 2844 | 0 | m-D1 | H | A-24 | A-24 | H | H |
| 2845 | 0 | m-D1 | H | A-25 | A-25 | H | H |
| 2846 | 0 | m-D1 | H | A-26 | A-26 | H | H |
| 2847 | 0 | m-D1 | H | A-27 | A-27 | H | H |
| 2848 | 0 | m-D1 | H | A-28 | A-28 | H | H |
| 2849 | 0 | m-D1 | H | A-29 | A-29 | H | H |
| 2850 | 0 | m-D1 | H | A-30 | A-30 | H | H |
| 2851 | 0 | m-D1 | H | A-31 | A-31 | H | H |
| 2852 | 0 | m-D1 | H | A-32 | A-32 | H | H |
| 2853 | 0 | m-D1 | H | A-33 | A-33 | H | H |
| 2854 | 0 | m-D1 | H | A-34 | A-34 | H | H |
| 2855 | 0 | m-D1 | H | A-35 | A-35 | H | H |
| 2856 | 0 | m-D1 | H | A-36 | A-36 | H | H |
| 2857 | 0 | m-D1 | H | A-37 | A-37 | H | H |
| 2858 | 0 | m-D1 | H | A-38 | A-38 | H | H |
| 2859 | 0 | m-D1 | H | A-39 | A-39 | H | H |
| 2860 | 0 | m-D1 | H | A-40 | A-40 | H | H |
| 2861 | 0 | m-D1 | H | A-41 | A-41 | H | H |
| 2862 | 0 | m-D1 | H | A-42 | A-42 | H | H |
| 2863 | 0 | m-D1 | H | A-43 | A-43 | H | H |
| 2864 | 0 | m-D1 | H | A-44 | A-44 | H | H |
| 2865 | 0 | m-D1 | H | A-45 | A-45 | H | H |
| 2866 | 0 | m-D1 | H | A-46 | A-46 | H | H |
| 2867 | 0 | m-D1 | H | A-47 | A-47 | H | H |
| 2868 | 0 | m-D2 | H | A-1 | A-1 | H | H |
| 2869 | 0 | m-D2 | H | A-2 | A-2 | H | H |
| 2870 | 0 | m-D2 | H | A-3 | A-3 | H | H |
| 2871 | 0 | m-D2 | H | A-4 | A-4 | H | H |
| 2872 | 0 | m-D2 | H | A-5 | A-5 | H | H |
| 2873 | 0 | m-D2 | H | A-6 | A-6 | H | H |
| 2874 | 0 | m-D2 | H | A-7 | A-7 | H | H |
| 2875 | 0 | m-D2 | H | A-8 | A-8 | H | H |
| 2876 | 0 | m-D2 | H | A-9 | A-9 | H | H |
| 2877 | 0 | m-D2 | H | A-10 | A-10 | H | H |
| 2878 | 0 | m-D2 | H | A-11 | A-11 | H | H |
| 2879 | 0 | m-D2 | H | A-12 | A-12 | H | H |
| 2880 | 0 | m-D2 | H | A-13 | A-13 | H | H |
| 2881 | 0 | m-D2 | H | A-14 | A-14 | H | H |
| 2882 | 0 | m-D2 | H | A-15 | A-15 | H | H |
| 2883 | 0 | m-D2 | H | A-16 | A-16 | H | H |
| 2884 | 0 | m-D2 | H | A-17 | A-17 | H | H |
| 2885 | 0 | m-D2 | H | A-18 | A-18 | H | H |
| 2886 | 0 | m-D2 | H | A-19 | A-19 | H | H |
| 2887 | 0 | m-D2 | H | A-20 | A-20 | H | H |
| 2888 | 0 | m-D2 | H | A-21 | A-21 | H | H |
| 2889 | 0 | m-D2 | H | A-22 | A-22 | H | H |
| 2890 | 0 | m-D2 | H | A-23 | A-23 | H | H |
| 2891 | 0 | m-D2 | H | A-24 | A-24 | H | H |
| 2892 | 0 | m-D2 | H | A-25 | A-25 | H | H |
| 2893 | 0 | m-D2 | H | A-26 | A-26 | H | H |
| 2894 | 0 | m-D2 | H | A-27 | A-27 | H | H |
| 2895 | 0 | m-D2 | H | A-28 | A-28 | H | H |
| 2896 | 0 | m-D2 | H | A-29 | A-29 | H | H |
| 2897 | 0 | m-D2 | H | A-30 | A-30 | H | H |
| 2898 | 0 | m-D2 | H | A-31 | A-31 | H | H |
| 2899 | 0 | m-D2 | H | A-32 | A-32 | H | H |
| 2900 | 0 | m-D2 | H | A-33 | A-33 | H | H |
| 2901 | 0 | m-D2 | H | A-34 | A-34 | H | H |
| 2902 | 0 | m-D2 | H | A-35 | A-35 | H | H |
| 2903 | 0 | m-D2 | H | A-36 | A-36 | H | H |
| 2904 | 0 | m-D2 | H | A-37 | A-37 | H | H |
| 2905 | 0 | m-D2 | H | A-38 | A-38 | H | H |
| 2906 | 0 | m-D2 | H | A-39 | A-39 | H | H |
| 2907 | 0 | m-D2 | H | A-40 | A-40 | H | H |
| 2908 | 0 | m-D2 | H | A-41 | A-41 | H | H |
| 2909 | 0 | m-D2 | H | A-42 | A-42 | H | H |
| 2910 | 0 | m-D2 | H | A-43 | A-43 | H | H |
| 2911 | 0 | m-D2 | H | A-44 | A-44 | H | H |
| 2912 | 0 | m-D2 | H | A-45 | A-45 | H | H |
| 2913 | 0 | m-D2 | H | A-46 | A-46 | H | H |
| 2914 | 0 | m-D2 | H | A-47 | A-47 | H | H |
| 2915 | 0 | m-D3 | H | A-1 | A-1 | H | H |
| 2916 | 0 | m-D3 | H | A-2 | A-2 | H | H |
| 2917 | 0 | m-D3 | H | A-3 | A-3 | H | H |
| 2918 | 0 | m-D3 | H | A-4 | A-4 | H | H |
| 2919 | 0 | m-D3 | H | A-5 | A-5 | H | H |
| 2920 | 0 | m-D3 | H | A-6 | A-6 | H | H |
| 2921 | 0 | m-D3 | H | A-7 | A-7 | H | H |
| 2922 | 0 | m-D3 | H | A-8 | A-8 | H | H |
| 2923 | 0 | m-D3 | H | A-9 | A-9 | H | H |
| 2924 | 0 | m-D3 | H | A-10 | A-10 | H | H |
| 2925 | 0 | m-D3 | H | A-11 | A-11 | H | H |
| 2926 | 0 | m-D3 | H | A-12 | A-12 | H | H |
| 2927 | 0 | m-D3 | H | A-13 | A-13 | H | H |
| 2928 | 0 | m-D3 | H | A-14 | A-14 | H | H |
| 2929 | 0 | m-D3 | H | A-15 | A-15 | H | H |
| 2930 | 0 | m-D3 | H | A-16 | A-16 | H | H |
| 2931 | 0 | m-D3 | H | A-17 | A-17 | H | H |
| 2932 | 0 | m-D3 | H | A-18 | A-18 | H | H |
| 2933 | 0 | m-D3 | H | A-19 | A-19 | H | H |
| 2934 | 0 | m-D3 | H | A-20 | A-20 | H | H |
| 2935 | 0 | m-D3 | H | A-21 | A-21 | H | H |
| 2936 | 0 | m-D3 | H | A-22 | A-22 | H | H |
| 2937 | 0 | m-D3 | H | A-23 | A-23 | H | H |
| 2938 | 0 | m-D3 | H | A-24 | A-24 | H | H |
| 2939 | 0 | m-D3 | H | A-25 | A-25 | H | H |
| 2940 | 0 | m-D3 | H | A-26 | A-26 | H | H |
| 2941 | 0 | m-D3 | H | A-27 | A-27 | H | H |
| 2942 | 0 | m-D3 | H | A-28 | A-28 | H | H |
| 2943 | 0 | m-D3 | H | A-29 | A-29 | H | H |
| 2944 | 0 | m-D3 | H | A-30 | A-30 | H | H |
| 2945 | 0 | m-D3 | H | A-31 | A-31 | H | H |
| 2946 | 0 | m-D3 | H | A-32 | A-32 | H | H |
| 2947 | 0 | m-D3 | H | A-33 | A-33 | H | H |
| 2948 | 0 | m-D3 | H | A-34 | A-34 | H | H |
| 2949 | 0 | m-D3 | H | A-35 | A-35 | H | H |
| 2950 | 0 | m-D3 | H | A-36 | A-36 | H | H |
| 2951 | 0 | m-D3 | H | A-37 | A-37 | H | H |
| 2952 | 0 | m-D3 | H | A-38 | A-38 | H | H |
| 2953 | 0 | m-D3 | H | A-39 | A-39 | H | H |
| 2954 | 0 | m-D3 | H | A-40 | A-40 | H | H |
| 2955 | 0 | m-D3 | H | A-41 | A-41 | H | H |
| 2956 | 0 | m-D3 | H | A-42 | A-42 | H | H |
| 2957 | 0 | m-D3 | H | A-43 | A-43 | H | H |
| 2958 | 0 | m-D3 | H | A-44 | A-44 | H | H |
| 2959 | 0 | m-D3 | H | A-45 | A-45 | H | H |
| 2960 | 0 | m-D3 | H | A-46 | A-46 | H | H |
| 2961 | 0 | m-D3 | H | A-47 | A-47 | H | H |
| 2962 | 0 | m-D5 | H | A-1 | A-1 | H | H |
| 2963 | 0 | m-D5 | H | A-2 | A-2 | H | H |
| 2964 | 0 | m-D5 | H | A-3 | A-3 | H | H |
| 2965 | 0 | m-D5 | H | A-4 | A-4 | H | H |
| 2966 | 0 | m-D5 | H | A-5 | A-5 | H | H |
| 2967 | 0 | m-D5 | H | A-6 | A-6 | H | H |
| 2968 | 0 | m-D5 | H | A-7 | A-7 | H | H |
| 2969 | 0 | m-D5 | H | A-8 | A-8 | H | H |
| 2970 | 0 | m-D5 | H | A-9 | A-9 | H | H |
| 2971 | 0 | m-D5 | H | A-10 | A-10 | H | H |
| 2972 | 0 | m-D5 | H | A-11 | A-11 | H | H |
| 2973 | 0 | m-D5 | H | A-12 | A-12 | H | H |
| 2974 | 0 | m-D5 | H | A-13 | A-13 | H | H |
| 2975 | 0 | m-D5 | H | A-14 | A-14 | H | H |
| 2976 | 0 | m-D5 | H | A-15 | A-15 | H | H |
| 2977 | 0 | m-D5 | H | A-16 | A-16 | H | H |
| 2978 | 0 | m-D5 | H | A-17 | A-17 | H | H |
| 2979 | 0 | m-D5 | H | A-18 | A-18 | H | H |
| 2980 | 0 | m-D5 | H | A-19 | A-19 | H | H |
| 2981 | 0 | m-D5 | H | A-20 | A-20 | H | H |
| 2982 | 0 | m-D5 | H | A-21 | A-21 | H | H |
| 2983 | 0 | m-D5 | H | A-22 | A-22 | H | H |
| 2984 | 0 | m-D5 | H | A-23 | A-23 | H | H |
| 2985 | 0 | m-D5 | H | A-24 | A-24 | H | H |
| 2986 | 0 | m-D5 | H | A-25 | A-25 | H | H |
| 2987 | 0 | m-D5 | H | A-26 | A-26 | H | H |
| 2988 | 0 | m-D5 | H | A-27 | A-27 | H | H |
| 2989 | 0 | m-D5 | H | A-28 | A-28 | H | H |
| 2990 | 0 | m-D5 | H | A-29 | A-29 | H | H |
| 2991 | 0 | m-D5 | H | A-30 | A-30 | H | H |
| 2992 | 0 | m-D5 | H | A-31 | A-31 | H | H |
| 2993 | 0 | m-D5 | H | A-32 | A-32 | H | H |
| 2994 | 0 | m-D5 | H | A-33 | A-33 | H | H |
| 2995 | 0 | m-D5 | H | A-34 | A-34 | H | H |
| 2996 | 0 | m-D5 | H | A-35 | A-35 | H | H |
| 2997 | 0 | m-D5 | H | A-36 | A-36 | H | H |
| 2998 | 0 | m-D5 | H | A-37 | A-37 | H | H |
| 2999 | 0 | m-D5 | H | A-38 | A-38 | H | H |
| 3000 | 0 | m-D5 | H | A-39 | A-39 | H | H |
| 3001 | 0 | m-D5 | H | A-40 | A-40 | H | H |
| 3002 | 0 | m-D5 | H | A-41 | A-41 | H | H |
| 3003 | 0 | m-D5 | H | A-42 | A-42 | H | H |
| 3004 | 0 | m-D5 | H | A-43 | A-43 | H | H |
| 3005 | 0 | m-D5 | H | A-44 | A-44 | H | H |
| 3006 | 0 | m-D5 | H | A-45 | A-45 | H | H |
| 3007 | 0 | m-D5 | H | A-46 | A-46 | H | H |
| 3008 | 0 | m-D5 | H | A-47 | A-47 | H | H |
| 3009 | 0 | m-D9 | H | A-1 | A-1 | H | H |
| 3010 | 0 | m-D9 | H | A-2 | A-2 | H | H |
| 3011 | 0 | m-D9 | H | A-3 | A-3 | H | H |
| 3012 | 0 | m-D9 | H | A-4 | A-4 | H | H |
| 3013 | 0 | m-D9 | H | A-5 | A-5 | H | H |
| 3014 | 0 | m-D9 | H | A-6 | A-6 | H | H |
| 3015 | 0 | m-D9 | H | A-7 | A-7 | H | H |
| 3016 | 0 | m-D9 | H | A-8 | A-8 | H | H |
| 3017 | 0 | m-D9 | H | A-9 | A-9 | H | H |
| 3018 | 0 | m-D9 | H | A-10 | A-10 | H | H |
| 3019 | 0 | m-D9 | H | A-11 | A-11 | H | H |
| 3020 | 0 | m-D9 | H | A-12 | A-12 | H | H |
| 3021 | 0 | m-D9 | H | A-13 | A-13 | H | H |
| 3022 | 0 | m-D9 | H | A-14 | A-14 | H | H |
| 3023 | 0 | m-D9 | H | A-15 | A-15 | H | H |
| 3024 | 0 | m-D9 | H | A-16 | A-16 | H | H |
| 3025 | 0 | m-D9 | H | A-17 | A-17 | H | H |
| 3026 | 0 | m-D9 | H | A-18 | A-18 | H | H |
| 3027 | 0 | m-D9 | H | A-19 | A-19 | H | H |
| 3028 | 0 | m-D9 | H | A-20 | A-20 | H | H |
| 3029 | 0 | m-D9 | H | A-21 | A-21 | H | H |
| 3030 | 0 | m-D9 | H | A-22 | A-22 | H | H |
| 3031 | 0 | m-D9 | H | A-23 | A-23 | H | H |
| 3032 | 0 | m-D9 | H | A-24 | A-24 | H | H |
| 3033 | 0 | m-D9 | H | A-25 | A-25 | H | H |
| 3034 | 0 | m-D9 | H | A-26 | A-26 | H | H |
| 3035 | 0 | m-D9 | H | A-27 | A-27 | H | H |
| 3036 | 0 | m-D9 | H | A-28 | A-28 | H | H |
| 3037 | 0 | m-D9 | H | A-29 | A-29 | H | H |
| 3038 | 0 | m-D9 | H | A-30 | A-30 | H | H |
| 3039 | 0 | m-D9 | H | A-31 | A-31 | H | H |
| 3040 | 0 | m-D9 | H | A-32 | A-32 | H | H |
| 3041 | 0 | m-D9 | H | A-33 | A-33 | H | H |
| 3042 | 0 | m-D9 | H | A-34 | A-34 | H | H |
| 3043 | 0 | m-D9 | H | A-35 | A-35 | H | H |
| 3044 | 0 | m-D9 | H | A-36 | A-36 | H | H |
| 3045 | 0 | m-D9 | H | A-37 | A-37 | H | H |
| 3046 | 0 | m-D9 | H | A-38 | A-38 | H | H |
| 3047 | 0 | m-D9 | H | A-39 | A-39 | H | H |
| 3048 | 0 | m-D9 | H | A-40 | A-40 | H | H |
| 3049 | 0 | m-D9 | H | A-41 | A-41 | H | H |
| 3050 | 0 | m-D9 | H | A-42 | A-42 | H | H |
| 3051 | 0 | m-D9 | H | A-43 | A-43 | H | H |
| 3052 | 0 | m-D9 | H | A-44 | A-44 | H | H |
| 3053 | 0 | m-D9 | H | A-45 | A-45 | H | H |
| 3054 | 0 | m-D9 | H | A-46 | A-46 | H | H |
| 3055 | 0 | m-D9 | H | A-47 | A-47 | H | H |
| 3056 | 0 | m-D11 | H | A-1 | A-1 | H | H |
| 3057 | 0 | m-D11 | H | A-2 | A-2 | H | H |
| 3058 | 0 | m-D11 | H | A-3 | A-3 | H | H |
| 3059 | 0 | m-D11 | H | A-4 | A-4 | H | H |
| 3060 | 0 | m-D11 | H | A-5 | A-5 | H | H |
| 3061 | 0 | m-D11 | H | A-6 | A-6 | H | H |
| 3062 | 0 | m-D11 | H | A-7 | A-7 | H | H |
| 3063 | 0 | m-D11 | H | A-8 | A-8 | H | H |
| 3064 | 0 | m-D11 | H | A-9 | A-9 | H | H |
| 3065 | 0 | m-D11 | H | A-10 | A-10 | H | H |
| 3066 | 0 | m-D11 | H | A-11 | A-11 | H | H |
| 3067 | 0 | m-D11 | H | A-12 | A-12 | H | H |
| 3068 | 0 | m-D11 | H | A-13 | A-13 | H | H |
| 3069 | 0 | m-D11 | H | A-14 | A-14 | H | H |
| 3070 | 0 | m-D11 | H | A-15 | A-15 | H | H |
| 3071 | 0 | m-D11 | H | A-16 | A-16 | H | H |
| 3072 | 0 | m-D11 | H | A-17 | A-17 | H | H |
| 3073 | 0 | m-D11 | H | A-18 | A-18 | H | H |
| 3074 | 0 | m-D11 | H | A-19 | A-19 | H | H |
| 3075 | 0 | m-D11 | H | A-20 | A-20 | H | H |
| 3076 | 0 | m-D11 | H | A-21 | A-21 | H | H |
| 3077 | 0 | m-D11 | H | A-22 | A-22 | H | H |
| 3078 | 0 | m-D11 | H | A-23 | A-23 | H | H |
| 3079 | 0 | m-D11 | H | A-24 | A-24 | H | H |
| 3080 | 0 | m-D11 | H | A-25 | A-25 | H | H |
| 3081 | 0 | m-D11 | H | A-26 | A-26 | H | H |
| 3082 | 0 | m-D11 | H | A-27 | A-27 | H | H |
| 3083 | 0 | m-D11 | H | A-28 | A-28 | H | H |
| 3084 | 0 | m-D11 | H | A-29 | A-29 | H | H |
| 3085 | 0 | m-D11 | H | A-30 | A-30 | H | H |
| 3086 | 0 | m-D11 | H | A-31 | A-31 | H | H |
| 3087 | 0 | m-D11 | H | A-32 | A-32 | H | H |
| 3088 | 0 | m-D11 | H | A-33 | A-33 | H | H |
| 3089 | 0 | m-D11 | H | A-34 | A-34 | H | H |
| 3090 | 0 | m-D11 | H | A-35 | A-35 | H | H |
| 3091 | 0 | m-D11 | H | A-36 | A-36 | H | H |
| 3092 | 0 | m-D11 | H | A-37 | A-37 | H | H |
| 3093 | 0 | m-D11 | H | A-38 | A-38 | H | H |
| 3094 | 0 | m-D11 | H | A-39 | A-39 | H | H |
| 3095 | 0 | m-D11 | H | A-40 | A-40 | H | H |
| 3096 | 0 | m-D11 | H | A-41 | A-41 | H | H |
| 3097 | 0 | m-D11 | H | A-42 | A-42 | H | H |
| 3098 | 0 | m-D11 | H | A-43 | A-43 | H | H |
| 3099 | 0 | m-D11 | H | A-44 | A-44 | H | H |
| 3100 | 0 | m-D11 | H | A-45 | A-45 | H | H |
| 3101 | 0 | m-D11 | H | A-46 | A-46 | H | H |
| 3102 | 0 | m-D11 | H | A-47 | A-47 | H | H |
| 3103 | 0 | m-D12 | H | A-1 | A-1 | H | H |
| 3104 | 0 | m-D12 | H | A-2 | A-2 | H | H |
| 3105 | 0 | m-D12 | H | A-3 | A-3 | H | H |
| 3106 | 0 | m-D12 | H | A-4 | A-4 | H | H |
| 3107 | 0 | m-D12 | H | A-5 | A-5 | H | H |
| 3108 | 0 | m-D12 | H | A-6 | A-6 | H | H |
| 3109 | 0 | m-D12 | H | A-7 | A-7 | H | H |
| 3110 | 0 | m-D12 | H | A-8 | A-8 | H | H |
| 3111 | 0 | m-D12 | H | A-9 | A-9 | H | H |
| 3112 | 0 | m-D12 | H | A-10 | A-10 | H | H |
| 3113 | 0 | m-D12 | H | A-11 | A-11 | H | H |
| 3114 | 0 | m-D12 | H | A-12 | A-12 | H | H |
| 3115 | 0 | m-D12 | H | A-13 | A-13 | H | H |
| 3116 | 0 | m-D12 | H | A-14 | A-14 | H | H |
| 3117 | 0 | m-D12 | H | A-15 | A-15 | H | H |
| 3118 | 0 | m-D12 | H | A-16 | A-16 | H | H |
| 3119 | 0 | m-D12 | H | A-17 | A-17 | H | H |
| 3120 | 0 | m-D12 | H | A-18 | A-18 | H | H |
| 3121 | 0 | m-D12 | H | A-19 | A-19 | H | H |
| 3122 | 0 | m-D12 | H | A-20 | A-20 | H | H |
| 3123 | 0 | m-D12 | H | A-21 | A-21 | H | H |
| 3124 | 0 | m-D12 | H | A-22 | A-22 | H | H |
| 3125 | 0 | m-D12 | H | A-23 | A-23 | H | H |
| 3126 | 0 | m-D12 | H | A-24 | A-24 | H | H |
| 3127 | 0 | m-D12 | H | A-25 | A-25 | H | H |
| 3128 | 0 | m-D12 | H | A-26 | A-26 | H | H |
| 3129 | 0 | m-D12 | H | A-27 | A-27 | H | H |
| 3130 | 0 | m-D12 | H | A-28 | A-28 | H | H |
| 3131 | 0 | m-D12 | H | A-29 | A-29 | H | H |
| 3132 | 0 | m-D12 | H | A-30 | A-30 | H | H |
| 3133 | 0 | m-D12 | H | A-31 | A-31 | H | H |
| 3134 | 0 | m-D12 | H | A-32 | A-32 | H | H |
| 3135 | 0 | m-D12 | H | A-33 | A-33 | H | H |
| 3136 | 0 | m-D12 | H | A-34 | A-34 | H | H |
| 3137 | 0 | m-D12 | H | A-35 | A-35 | H | H |
| 3138 | 0 | m-D12 | H | A-36 | A-36 | H | H |
| 3139 | 0 | m-D12 | H | A-37 | A-37 | H | H |
| 3140 | 0 | m-D12 | H | A-38 | A-38 | H | H |
| 3141 | 0 | m-D12 | H | A-39 | A-39 | H | H |
| 3142 | 0 | m-D12 | H | A-40 | A-40 | H | H |
| 3143 | 0 | m-D12 | H | A-41 | A-41 | H | H |
| 3144 | 0 | m-D12 | H | A-42 | A-42 | H | H |
| 3145 | 0 | m-D12 | H | A-43 | A-43 | H | H |
| 3146 | 0 | m-D12 | H | A-44 | A-44 | H | H |
| 3147 | 0 | m-D12 | H | A-45 | A-45 | H | H |
| 3148 | 0 | m-D12 | H | A-46 | A-46 | H | H |
| 3149 | 0 | m-D12 | H | A-47 | A-47 | H | H |
| 3150 | 0 | m-D13 | H | A-1 | A-1 | H | H |
| 3151 | 0 | m-D13 | H | A-2 | A-2 | H | H |
| 3152 | 0 | m-D13 | H | A-3 | A-3 | H | H |
| 3153 | 0 | m-D13 | H | A-4 | A-4 | H | H |
| 3154 | 0 | m-D13 | H | A-5 | A-5 | H | H |
| 3155 | 0 | m-D13 | H | A-6 | A-6 | H | H |
| 3156 | 0 | m-D13 | H | A-7 | A-7 | H | H |
| 3157 | 0 | m-D13 | H | A-8 | A-8 | H | H |
| 3158 | 0 | m-D13 | H | A-9 | A-9 | H | H |
| 3159 | 0 | m-D13 | H | A-10 | A-10 | H | H |
| 3160 | 0 | m-D13 | H | A-11 | A-11 | H | H |
| 3161 | 0 | m-D13 | H | A-12 | A-12 | H | H |
| 3162 | 0 | m-D13 | H | A-13 | A-13 | H | H |
| 3163 | 0 | m-D13 | H | A-14 | A-14 | H | H |
| 3164 | 0 | m-D13 | H | A-15 | A-15 | H | H |
| 3165 | 0 | m-D13 | H | A-16 | A-16 | H | H |
| 3166 | 0 | m-D13 | H | A-17 | A-17 | H | H |
| 3167 | 0 | m-D13 | H | A-18 | A-18 | H | H |
| 3168 | 0 | m-D13 | H | A-19 | A-19 | H | H |
| 3169 | 0 | m-D13 | H | A-20 | A-20 | H | H |
| 3170 | 0 | m-D13 | H | A-21 | A-21 | H | H |
| 3171 | 0 | m-D13 | H | A-22 | A-22 | H | H |
| 3172 | 0 | m-D13 | H | A-23 | A-23 | H | H |
| 3173 | 0 | m-D13 | H | A-24 | A-24 | H | H |
| 3174 | 0 | m-D13 | H | A-25 | A-25 | H | H |
| 3175 | 0 | m-D13 | H | A-26 | A-26 | H | H |
| 3176 | 0 | m-D13 | H | A-27 | A-27 | H | H |
| 3177 | 0 | m-D13 | H | A-28 | A-28 | H | H |
| 3178 | 0 | m-D13 | H | A-29 | A-29 | H | H |
| 3179 | 0 | m-D13 | H | A-30 | A-30 | H | H |
| 3180 | 0 | m-D13 | H | A-31 | A-31 | H | H |
| 3181 | 0 | m-D13 | H | A-32 | A-32 | H | H |
| 3182 | 0 | m-D13 | H | A-33 | A-33 | H | H |
| 3183 | 0 | m-D13 | H | A-34 | A-34 | H | H |
| 3184 | 0 | m-D13 | H | A-35 | A-35 | H | H |
| 3185 | 0 | m-D13 | H | A-36 | A-36 | H | H |
| 3186 | 0 | m-D13 | H | A-37 | A-37 | H | H |
| 3187 | 0 | m-D13 | H | A-38 | A-38 | H | H |
| 3188 | 0 | m-D13 | H | A-39 | A-39 | H | H |
| 3189 | 0 | m-D13 | H | A-40 | A-40 | H | H |
| 3190 | 0 | m-D13 | H | A-41 | A-41 | H | H |
| 3191 | 0 | m-D13 | H | A-42 | A-42 | H | H |
| 3192 | 0 | m-D13 | H | A-43 | A-43 | H | H |
| 3193 | 0 | m-D13 | H | A-44 | A-44 | H | H |
| 3194 | 0 | m-D13 | H | A-45 | A-45 | H | H |
| 3195 | 0 | m-D13 | H | A-46 | A-46 | H | H |
| 3196 | 0 | m-D13 | H | A-47 | A-47 | H | H |
| 3197 | 0 | m-D14 | H | A-1 | A-1 | H | H |
| 3198 | 0 | m-D14 | H | A-2 | A-2 | H | H |
| 3199 | 0 | m-D14 | H | A-3 | A-3 | H | H |
| 3200 | 0 | m-D14 | H | A-4 | A-4 | H | H |
| 3201 | 0 | m-D14 | H | A-5 | A-5 | H | H |
| 3202 | 0 | m-D14 | H | A-6 | A-6 | H | H |
| 3203 | 0 | m-D14 | H | A-7 | A-7 | H | H |
| 3204 | 0 | m-D14 | H | A-8 | A-8 | H | H |
| 3205 | 0 | m-D14 | H | A-9 | A-9 | H | H |
| 3206 | 0 | m-D14 | H | A-10 | A-10 | H | H |
| 3207 | 0 | m-D14 | H | A-11 | A-11 | H | H |
| 3208 | 0 | m-D14 | H | A-12 | A-12 | H | H |
| 3209 | 0 | m-D14 | H | A-13 | A-13 | H | H |
| 3210 | 0 | m-D14 | H | A-14 | A-14 | H | H |
| 3211 | 0 | m-D14 | H | A-15 | A-15 | H | H |
| 3212 | 0 | m-D14 | H | A-16 | A-16 | H | H |
| 3213 | 0 | m-D14 | H | A-17 | A-17 | H | H |
| 3214 | 0 | m-D14 | H | A-18 | A-18 | H | H |
| 3215 | 0 | m-D14 | H | A-19 | A-19 | H | H |
| 3216 | 0 | m-D14 | H | A-20 | A-20 | H | H |
| 3217 | 0 | m-D14 | H | A-21 | A-21 | H | H |
| 3218 | 0 | m-D14 | H | A-22 | A-22 | H | H |
| 3219 | 0 | m-D14 | H | A-23 | A-23 | H | H |
| 3220 | 0 | m-D14 | H | A-24 | A-24 | H | H |
| 3221 | 0 | m-D14 | H | A-25 | A-25 | H | H |
| 3222 | 0 | m-D14 | H | A-26 | A-26 | H | H |
| 3223 | 0 | m-D14 | H | A-27 | A-27 | H | H |
| 3224 | 0 | m-D14 | H | A-28 | A-28 | H | H |
| 3225 | 0 | m-D14 | H | A-29 | A-29 | H | H |
| 3226 | 0 | m-D14 | H | A-30 | A-30 | H | H |
| 3227 | 0 | m-D14 | H | A-31 | A-31 | H | H |
| 3228 | 0 | m-D14 | H | A-32 | A-32 | H | H |
| 3229 | 0 | m-D14 | H | A-33 | A-33 | H | H |
| 3230 | 0 | m-D14 | H | A-34 | A-34 | H | H |
| 3231 | 0 | m-D14 | H | A-35 | A-35 | H | H |
| 3232 | 0 | m-D14 | H | A-36 | A-36 | H | H |
| 3233 | 0 | m-D14 | H | A-37 | A-37 | H | H |
| 3234 | 0 | m-D14 | H | A-38 | A-38 | H | H |
| 3235 | 0 | m-D14 | H | A-39 | A-39 | H | H |
| 3236 | 0 | m-D14 | H | A-40 | A-40 | H | H |
| 3237 | 0 | m-D14 | H | A-41 | A-41 | H | H |
| 3238 | 0 | m-D14 | H | A-42 | A-42 | H | H |
| 3239 | 0 | m-D14 | H | A-43 | A-43 | H | H |
| 3240 | 0 | m-D14 | H | A-44 | A-44 | H | H |
| 3241 | 0 | m-D14 | H | A-45 | A-45 | H | H |
| 3242 | 0 | m-D14 | H | A-46 | A-46 | H | H |
| 3243 | 0 | m-D14 | H | A-47 | A-47 | H | H |
| 3244 | 0 | m-D1 | H | A-1 | m-D1 | H | H |
| 3245 | 0 | m-D1 | H | A-2 | m-D1 | H | H |
| 3246 | 0 | m-D1 | H | A-3 | m-D1 | H | H |
| 3247 | 0 | m-D1 | H | A-4 | m-D1 | H | H |
| 3248 | 0 | m-D1 | H | A-5 | m-D1 | H | H |
| 3249 | 0 | m-D1 | H | A-6 | m-D1 | H | H |
| 3250 | 0 | m-D1 | H | A-7 | m-D1 | H | H |
| 3251 | 0 | m-D1 | H | A-8 | m-D1 | H | H |
| 3252 | 0 | m-D1 | H | A-9 | m-D1 | H | H |
| 3253 | 0 | m-D1 | H | A-10 | m-D1 | H | H |
| 3254 | 0 | m-D1 | H | A-11 | m-D1 | H | H |
| 3255 | 0 | m-D1 | H | A-12 | m-D1 | H | H |
| 3256 | 0 | m-D1 | H | A-13 | m-D1 | H | H |
| 3257 | 0 | m-D1 | H | A-14 | m-D1 | H | H |
| 3258 | 0 | m-D1 | H | A-15 | m-D1 | H | H |
| 3259 | 0 | m-D1 | H | A-16 | m-D1 | H | H |
| 3260 | 0 | m-D1 | H | A-17 | m-D1 | H | H |
| 3261 | 0 | m-D1 | H | A-18 | m-D1 | H | H |
| 3262 | 0 | m-D1 | H | A-19 | m-D1 | H | H |
| 3263 | 0 | m-D1 | H | A-20 | m-D1 | H | H |
| 3264 | 0 | m-D1 | H | A-21 | m-D1 | H | H |
| 3265 | 0 | m-D1 | H | A-22 | m-D1 | H | H |
| 3266 | 0 | m-D1 | H | A-23 | m-D1 | H | H |
| 3267 | 0 | m-D1 | H | A-24 | m-D1 | H | H |
| 3268 | 0 | m-D1 | H | A-25 | m-D1 | H | H |
| 3269 | 0 | m-D1 | H | A-26 | m-D1 | H | H |
| 3270 | 0 | m-D1 | H | A-27 | m-D1 | H | H |
| 3271 | 0 | m-D1 | H | A-28 | m-D1 | H | H |
| 3272 | 0 | m-D1 | H | A-29 | m-D1 | H | H |
| 3273 | 0 | m-D1 | H | A-30 | m-D1 | H | H |
| 3274 | 0 | m-D1 | H | A-31 | m-D1 | H | H |
| 3275 | 0 | m-D1 | H | A-32 | m-D1 | H | H |
| 3276 | 0 | m-D1 | H | A-33 | m-D1 | H | H |
| 3277 | 0 | m-D1 | H | A-34 | m-D1 | H | H |
| 3278 | 0 | m-D1 | H | A-35 | m-D1 | H | H |
| 3279 | 0 | m-D1 | H | A-36 | m-D1 | H | H |
| 3280 | 0 | m-D1 | H | A-37 | m-D1 | H | H |
| 3281 | 0 | m-D1 | H | A-38 | m-D1 | H | H |
| 3282 | 0 | m-D1 | H | A-39 | m-D1 | H | H |
| 3283 | 0 | m-D1 | H | A-40 | m-D1 | H | H |
| 3284 | 0 | m-D1 | H | A-41 | m-D1 | H | H |
| 3285 | 0 | m-D1 | H | A-42 | m-D1 | H | H |
| 3286 | 0 | m-D1 | H | A-43 | m-D1 | H | H |
| 3287 | 0 | m-D1 | H | A-44 | m-D1 | H | H |
| 3288 | 0 | m-D1 | H | A-45 | m-D1 | H | H |
| 3289 | 0 | m-D1 | H | A-46 | m-D1 | H | H |
| 3290 | 0 | m-D1 | H | A-47 | m-D1 | H | H |
| 3291 | 0 | m-D2 | H | A-1 | m-D2 | H | H |
| 3292 | 0 | m-D2 | H | A-2 | m-D2 | H | H |
| 3293 | 0 | m-D2 | H | A-3 | m-D2 | H | H |
| 3294 | 0 | m-D2 | H | A-4 | m-D2 | H | H |
| 3295 | 0 | m-D2 | H | A-5 | m-D2 | H | H |
| 3296 | 0 | m-D2 | H | A-6 | m-D2 | H | H |
| 3297 | 0 | m-D2 | H | A-7 | m-D2 | H | H |
| 3298 | 0 | m-D2 | H | A-8 | m-D2 | H | H |
| 3299 | 0 | m-D2 | H | A-9 | m-D2 | H | H |
| 3300 | 0 | m-D2 | H | A-10 | m-D2 | H | H |
| 3301 | 0 | m-D2 | H | A-11 | m-D2 | H | H |
| 3302 | 0 | m-D2 | H | A-12 | m-D2 | H | H |
| 3303 | 0 | m-D2 | H | A-13 | m-D2 | H | H |
| 3304 | 0 | m-D2 | H | A-14 | m-D2 | H | H |
| 3305 | 0 | m-D2 | H | A-15 | m-D2 | H | H |
| 3306 | 0 | m-D2 | H | A-16 | m-D2 | H | H |
| 3307 | 0 | m-D2 | H | A-17 | m-D2 | H | H |
| 3308 | 0 | m-D2 | H | A-18 | m-D2 | H | H |
| 3309 | 0 | m-D2 | H | A-19 | m-D2 | H | H |
| 3310 | 0 | m-D2 | H | A-20 | m-D2 | H | H |
| 3311 | 0 | m-D2 | H | A-21 | m-D2 | H | H |
| 3312 | 0 | m-D2 | H | A-22 | m-D2 | H | H |
| 3313 | 0 | m-D2 | H | A-23 | m-D2 | H | H |
| 3314 | 0 | m-D2 | H | A-24 | m-D2 | H | H |
| 3315 | 0 | m-D2 | H | A-25 | m-D2 | H | H |
| 3316 | 0 | m-D2 | H | A-26 | m-D2 | H | H |
| 3317 | 0 | m-D2 | H | A-27 | m-D2 | H | H |
| 3318 | 0 | m-D2 | H | A-28 | m-D2 | H | H |
| 3319 | 0 | m-D2 | H | A-29 | m-D2 | H | H |
| 3320 | 0 | m-D2 | H | A-30 | m-D2 | H | H |
| 3321 | 0 | m-D2 | H | A-31 | m-D2 | H | H |
| 3322 | 0 | m-D2 | H | A-32 | m-D2 | H | H |
| 3323 | 0 | m-D2 | H | A-33 | m-D2 | H | H |
| 3324 | 0 | m-D2 | H | A-34 | m-D2 | H | H |
| 3325 | 0 | m-D2 | H | A-35 | m-D2 | H | H |
| 3326 | 0 | m-D2 | H | A-36 | m-D2 | H | H |
| 3327 | 0 | m-D2 | H | A-37 | m-D2 | H | H |
| 3328 | 0 | m-D2 | H | A-38 | m-D2 | H | H |
| 3329 | 0 | m-D2 | H | A-39 | m-D2 | H | H |
| 3330 | 0 | m-D2 | H | A-40 | m-D2 | H | H |
| 3331 | 0 | m-D2 | H | A-41 | m-D2 | H | H |
| 3332 | 0 | m-D2 | H | A-42 | m-D2 | H | H |
| 3333 | 0 | m-D2 | H | A-43 | m-D2 | H | H |
| 3334 | 0 | m-D2 | H | A-44 | m-D2 | H | H |
| 3335 | 0 | m-D2 | H | A-45 | m-D2 | H | H |
| 3336 | 0 | m-D2 | H | A-46 | m-D2 | H | H |
| 3337 | 0 | m-D2 | H | A-47 | m-D2 | H | H |
| 3338 | 0 | m-D3 | H | A-1 | m-D3 | H | H |
| 3339 | 0 | m-D3 | H | A-2 | m-D3 | H | H |
| 3340 | 0 | m-D3 | H | A-3 | m-D3 | H | H |
| 3341 | 0 | m-D3 | H | A-4 | m-D3 | H | H |
| 3342 | 0 | m-D3 | H | A-5 | m-D3 | H | H |
| 3343 | 0 | m-D3 | H | A-6 | m-D3 | H | H |
| 3344 | 0 | m-D3 | H | A-7 | m-D3 | H | H |
| 3345 | 0 | m-D3 | H | A-8 | m-D3 | H | H |
| 3346 | 0 | m-D3 | H | A-9 | m-D3 | H | H |
| 3347 | 0 | m-D3 | H | A-10 | m-D3 | H | H |
| 3348 | 0 | m-D3 | H | A-11 | m-D3 | H | H |
| 3349 | 0 | m-D3 | H | A-12 | m-D3 | H | H |
| 3350 | 0 | m-D3 | H | A-13 | m-D3 | H | H |
| 3351 | 0 | m-D3 | H | A-14 | m-D3 | H | H |
| 3352 | 0 | m-D3 | H | A-15 | m-D3 | H | H |
| 3353 | 0 | m-D3 | H | A-16 | m-D3 | H | H |
| 3354 | 0 | m-D3 | H | A-17 | m-D3 | H | H |
| 3355 | 0 | m-D3 | H | A-18 | m-D3 | H | H |
| 3356 | 0 | m-D3 | H | A-19 | m-D3 | H | H |
| 3357 | 0 | m-D3 | H | A-20 | m-D3 | H | H |
| 3358 | 0 | m-D3 | H | A-21 | m-D3 | H | H |
| 3359 | 0 | m-D3 | H | A-22 | m-D3 | H | H |
| 3360 | 0 | m-D3 | H | A-23 | m-D3 | H | H |
| 3361 | 0 | m-D3 | H | A-24 | m-D3 | H | H |
| 3362 | 0 | m-D3 | H | A-25 | m-D3 | H | H |
| 3363 | 0 | m-D3 | H | A-26 | m-D3 | H | H |
| 3364 | 0 | m-D3 | H | A-27 | m-D3 | H | H |
| 3365 | 0 | m-D3 | H | A-28 | m-D3 | H | H |
| 3366 | 0 | m-D3 | H | A-29 | m-D3 | H | H |
| 3367 | 0 | m-D3 | H | A-30 | m-D3 | H | H |
| 3368 | 0 | m-D3 | H | A-31 | m-D3 | H | H |
| 3369 | 0 | m-D3 | H | A-32 | m-D3 | H | H |
| 3370 | 0 | m-D3 | H | A-33 | m-D3 | H | H |
| 3371 | 0 | m-D3 | H | A-34 | m-D3 | H | H |
| 3372 | 0 | m-D3 | H | A-35 | m-D3 | H | H |
| 3373 | 0 | m-D3 | H | A-36 | m-D3 | H | H |
| 3374 | 0 | m-D3 | H | A-37 | m-D3 | H | H |
| 3375 | 0 | m-D3 | H | A-38 | m-D3 | H | H |
| 3376 | 0 | m-D3 | H | A-39 | m-D3 | H | H |
| 3377 | 0 | m-D3 | H | A-40 | m-D3 | H | H |
| 3378 | 0 | m-D3 | H | A-41 | m-D3 | H | H |
| 3379 | 0 | m-D3 | H | A-42 | m-D3 | H | H |
| 3380 | 0 | m-D3 | H | A-43 | m-D3 | H | H |
| 3381 | 0 | m-D3 | H | A-44 | m-D3 | H | H |
| 3382 | 0 | m-D3 | H | A-45 | m-D3 | H | H |
| 3383 | 0 | m-D3 | H | A-46 | m-D3 | H | H |
| 3384 | 0 | m-D3 | H | A-47 | m-D3 | H | H |
| 3385 | 0 | m-D5 | H | A-1 | m-D5 | H | H |
| 3386 | 0 | m-D5 | H | A-2 | m-D5 | H | H |
| 3387 | 0 | m-D5 | H | A-3 | m-D5 | H | H |
| 3388 | 0 | m-D5 | H | A-4 | m-D5 | H | H |
| 3389 | 0 | m-D5 | H | A-5 | m-D5 | H | H |
| 3390 | 0 | m-D5 | H | A-6 | m-D5 | H | H |
| 3391 | 0 | m-D5 | H | A-7 | m-D5 | H | H |
| 3392 | 0 | m-D5 | H | A-8 | m-D5 | H | H |
| 3393 | 0 | m-D5 | H | A-9 | m-D5 | H | H |
| 3394 | 0 | m-D5 | H | A-10 | m-D5 | H | H |
| 3395 | 0 | m-D5 | H | A-11 | m-D5 | H | H |
| 3396 | 0 | m-D5 | H | A-12 | m-D5 | H | H |
| 3397 | 0 | m-D5 | H | A-13 | m-D5 | H | H |
| 3398 | 0 | m-D5 | H | A-14 | m-D5 | H | H |
| 3399 | 0 | m-D5 | H | A-15 | m-D5 | H | H |
| 3400 | 0 | m-D5 | H | A-16 | m-D5 | H | H |
| 3401 | 0 | m-D5 | H | A-17 | m-D5 | H | H |
| 3402 | 0 | m-D5 | H | A-18 | m-D5 | H | H |
| 3403 | 0 | m-D5 | H | A-19 | m-D5 | H | H |
| 3404 | 0 | m-D5 | H | A-20 | m-D5 | H | H |
| 3405 | 0 | m-D5 | H | A-21 | m-D5 | H | H |
| 3406 | 0 | m-D5 | H | A-22 | m-D5 | H | H |
| 3407 | 0 | m-D5 | H | A-23 | m-D5 | H | H |
| 3408 | 0 | m-D5 | H | A-24 | m-D5 | H | H |
| 3409 | 0 | m-D5 | H | A-25 | m-D5 | H | H |
| 3410 | 0 | m-D5 | H | A-26 | m-D5 | H | H |
| 3411 | 0 | m-D5 | H | A-27 | m-D5 | H | H |
| 3412 | 0 | m-D5 | H | A-28 | m-D5 | H | H |
| 3413 | 0 | m-D5 | H | A-29 | m-D5 | H | H |
| 3414 | 0 | m-D5 | H | A-30 | m-D5 | H | H |
| 3415 | 0 | m-D5 | H | A-31 | m-D5 | H | H |
| 3416 | 0 | m-D5 | H | A-32 | m-D5 | H | H |
| 3417 | 0 | m-D5 | H | A-33 | m-D5 | H | H |
| 3418 | 0 | m-D5 | H | A-34 | m-D5 | H | H |
| 3419 | 0 | m-D5 | H | A-35 | m-D5 | H | H |
| 3420 | 0 | m-D5 | H | A-36 | m-D5 | H | H |
| 3421 | 0 | m-D5 | H | A-37 | m-D5 | H | H |
| 3422 | 0 | m-D5 | H | A-38 | m-D5 | H | H |
| 3423 | 0 | m-D5 | H | A-39 | m-D5 | H | H |
| 3424 | 0 | m-D5 | H | A-40 | m-D5 | H | H |
| 3425 | 0 | m-D5 | H | A-41 | m-D5 | H | H |
| 3426 | 0 | m-D5 | H | A-42 | m-D5 | H | H |
| 3427 | 0 | m-D5 | H | A-43 | m-D5 | H | H |
| 3428 | 0 | m-D5 | H | A-44 | m-D5 | H | H |
| 3429 | 0 | m-D5 | H | A-45 | m-D5 | H | H |
| 3430 | 0 | m-D5 | H | A-46 | m-D5 | H | H |
| 3431 | 0 | m-D5 | H | A-47 | m-D5 | H | H |
| 3432 | 0 | m-D9 | H | A-1 | m-D9 | H | H |
| 3433 | 0 | m-D9 | H | A-2 | m-D9 | H | H |
| 3434 | 0 | m-D9 | H | A-3 | m-D9 | H | H |
| 3435 | 0 | m-D9 | H | A-4 | m-D9 | H | H |
| 3436 | 0 | m-D9 | H | A-5 | m-D9 | H | H |
| 3437 | 0 | m-D9 | H | A-6 | m-D9 | H | H |
| 3438 | 0 | m-D9 | H | A-7 | m-D9 | H | H |
| 3439 | 0 | m-D9 | H | A-8 | m-D9 | H | H |
| 3440 | 0 | m-D9 | H | A-9 | m-D9 | H | H |
| 3441 | 0 | m-D9 | H | A-10 | m-D9 | H | H |
| 3442 | 0 | m-D9 | H | A-11 | m-D9 | H | H |
| 3443 | 0 | m-D9 | H | A-12 | m-D9 | H | H |
| 3444 | 0 | m-D9 | H | A-13 | m-D9 | H | H |
| 3445 | 0 | m-D9 | H | A-14 | m-D9 | H | H |
| 3446 | 0 | m-D9 | H | A-15 | m-D9 | H | H |
| 3447 | 0 | m-D9 | H | A-16 | m-D9 | H | H |
| 3448 | 0 | m-D9 | H | A-17 | m-D9 | H | H |
| 3449 | 0 | m-D9 | H | A-18 | m-D9 | H | H |
| 3450 | 0 | m-D9 | H | A-19 | m-D9 | H | H |
| 3451 | 0 | m-D9 | H | A-20 | m-D9 | H | H |
| 3452 | 0 | m-D9 | H | A-21 | m-D9 | H | H |
| 3453 | 0 | m-D9 | H | A-22 | m-D9 | H | H |
| 3454 | 0 | m-D9 | H | A-23 | m-D9 | H | H |
| 3455 | 0 | m-D9 | H | A-24 | m-D9 | H | H |
| 3456 | 0 | m-D9 | H | A-25 | m-D9 | H | H |
| 3457 | 0 | m-D9 | H | A-26 | m-D9 | H | H |
| 3458 | 0 | m-D9 | H | A-27 | m-D9 | H | H |
| 3459 | 0 | m-D9 | H | A-28 | m-D9 | H | H |
| 3460 | 0 | m-D9 | H | A-29 | m-D9 | H | H |
| 3461 | 0 | m-D9 | H | A-30 | m-D9 | H | H |
| 3462 | 0 | m-D9 | H | A-31 | m-D9 | H | H |
| 3463 | 0 | m-D9 | H | A-32 | m-D9 | H | H |
| 3464 | 0 | m-D9 | H | A-33 | m-D9 | H | H |
| 3465 | 0 | m-D9 | H | A-34 | m-D9 | H | H |
| 3466 | 0 | m-D9 | H | A-35 | m-D9 | H | H |
| 3467 | 0 | m-D9 | H | A-36 | m-D9 | H | H |
| 3468 | 0 | m-D9 | H | A-37 | m-D9 | H | H |
| 3469 | 0 | m-D9 | H | A-38 | m-D9 | H | H |
| 3470 | 0 | m-D9 | H | A-39 | m-D9 | H | H |
| 3471 | 0 | m-D9 | H | A-40 | m-D9 | H | H |
| 3472 | 0 | m-D9 | H | A-41 | m-D9 | H | H |
| 3473 | 0 | m-D9 | H | A-42 | m-D9 | H | H |
| 3474 | 0 | m-D9 | H | A-43 | m-D9 | H | H |
| 3475 | 0 | m-D9 | H | A-44 | m-D9 | H | H |
| 3476 | 0 | m-D9 | H | A-45 | m-D9 | H | H |
| 3477 | 0 | m-D9 | H | A-46 | m-D9 | H | H |
| 3478 | 0 | m-D9 | H | A-47 | m-D9 | H | H |
| 3479 | 0 | m-D11 | H | A-1 | m-D11 | H | H |
| 3480 | 0 | m-D11 | H | A-2 | m-D11 | H | H |
| 3481 | 0 | m-D11 | H | A-3 | m-D11 | H | H |
| 3482 | 0 | m-D11 | H | A-4 | m-D11 | H | H |
| 3483 | 0 | m-D11 | H | A-5 | m-D11 | H | H |
| 3484 | 0 | m-D11 | H | A-6 | m-D11 | H | H |
| 3485 | 0 | m-D11 | H | A-7 | m-D11 | H | H |
| 3486 | 0 | m-D11 | H | A-8 | m-D11 | H | H |
| 3487 | 0 | m-D11 | H | A-9 | m-D11 | H | H |
| 3488 | 0 | m-D11 | H | A-10 | m-D11 | H | H |
| 3489 | 0 | m-D11 | H | A-11 | m-D11 | H | H |
| 3490 | 0 | m-D11 | H | A-12 | m-D11 | H | H |
| 3491 | 0 | m-D11 | H | A-13 | m-D11 | H | H |
| 3492 | 0 | m-D11 | H | A-14 | m-D11 | H | H |
| 3493 | 0 | m-D11 | H | A-15 | m-D11 | H | H |
| 3494 | 0 | m-D11 | H | A-16 | m-D11 | H | H |
| 3495 | 0 | m-D11 | H | A-17 | m-D11 | H | H |
| 3496 | 0 | m-D11 | H | A-18 | m-D11 | H | H |
| 3497 | 0 | m-D11 | H | A-19 | m-D11 | H | H |
| 3498 | 0 | m-D11 | H | A-20 | m-D11 | H | H |
| 3499 | 0 | m-D11 | H | A-21 | m-D11 | H | H |
| 3500 | 0 | m-D11 | H | A-22 | m-D11 | H | H |
| 3501 | 0 | m-D11 | H | A-23 | m-D11 | H | H |
| 3502 | 0 | m-D11 | H | A-24 | m-D11 | H | H |
| 3503 | 0 | m-D11 | H | A-25 | m-D11 | H | H |
| 3504 | 0 | m-D11 | H | A-26 | m-D11 | H | H |
| 3505 | 0 | m-D11 | H | A-27 | m-D11 | H | H |
| 3506 | 0 | m-D11 | H | A-28 | m-D11 | H | H |
| 3507 | 0 | m-D11 | H | A-29 | m-D11 | H | H |
| 3508 | 0 | m-D11 | H | A-30 | m-D11 | H | H |
| 3509 | 0 | m-D11 | H | A-31 | m-D11 | H | H |
| 3510 | 0 | m-D11 | H | A-32 | m-D11 | H | H |
| 3511 | 0 | m-D11 | H | A-33 | m-D11 | H | H |
| 3512 | 0 | m-D11 | H | A-34 | m-D11 | H | H |
| 3513 | 0 | m-D11 | H | A-35 | m-D11 | H | H |
| 3514 | 0 | m-D11 | H | A-36 | m-D11 | H | H |
| 3515 | 0 | m-D11 | H | A-37 | m-D11 | H | H |
| 3516 | 0 | m-D11 | H | A-38 | m-D11 | H | H |
| 3517 | 0 | m-D11 | H | A-39 | m-D11 | H | H |
| 3518 | 0 | m-D11 | H | A-40 | m-D11 | H | H |
| 3519 | 0 | m-D11 | H | A-41 | m-D11 | H | H |
| 3520 | 0 | m-D11 | H | A-42 | m-D11 | H | H |
| 3521 | 0 | m-D11 | H | A-43 | m-D11 | H | H |
| 3522 | 0 | m-D11 | H | A-44 | m-D11 | H | H |
| 3523 | 0 | m-D11 | H | A-45 | m-D11 | H | H |
| 3524 | 0 | m-D11 | H | A-46 | m-D11 | H | H |
| 3525 | 0 | m-D11 | H | A-47 | m-D11 | H | H |
| 3526 | 0 | m-D12 | H | A-1 | m-D12 | H | H |
| 3527 | 0 | m-D12 | H | A-2 | m-D12 | H | H |
| 3528 | 0 | m-D12 | H | A-3 | m-D12 | H | H |
| 3529 | 0 | m-D12 | H | A-4 | m-D12 | H | H |
| 3530 | 0 | m-D12 | H | A-5 | m-D12 | H | H |
| 3531 | 0 | m-D12 | H | A-6 | m-D12 | H | H |
| 3532 | 0 | m-D12 | H | A-7 | m-D12 | H | H |
| 3533 | 0 | m-D12 | H | A-8 | m-D12 | H | H |
| 3534 | 0 | m-D12 | H | A-9 | m-D12 | H | H |
| 3535 | 0 | m-D12 | H | A-10 | m-D12 | H | H |
| 3536 | 0 | m-D12 | H | A-11 | m-D12 | H | H |
| 3537 | 0 | m-D12 | H | A-12 | m-D12 | H | H |
| 3538 | 0 | m-D12 | H | A-13 | m-D12 | H | H |
| 3539 | 0 | m-D12 | H | A-14 | m-D12 | H | H |
| 3540 | 0 | m-D12 | H | A-15 | m-D12 | H | H |
| 3541 | 0 | m-D12 | H | A-16 | m-D12 | H | H |
| 3542 | 0 | m-D12 | H | A-17 | m-D12 | H | H |
| 3543 | 0 | m-D12 | H | A-18 | m-D12 | H | H |
| 3544 | 0 | m-D12 | H | A-19 | m-D12 | H | H |
| 3545 | 0 | m-D12 | H | A-20 | m-D12 | H | H |
| 3546 | 0 | m-D12 | H | A-21 | m-D12 | H | H |
| 3547 | 0 | m-D12 | H | A-22 | m-D12 | H | H |
| 3548 | 0 | m-D12 | H | A-23 | m-D12 | H | H |
| 3549 | 0 | m-D12 | H | A-24 | m-D12 | H | H |
| 3550 | 0 | m-D12 | H | A-25 | m-D12 | H | H |
| 3551 | 0 | m-D12 | H | A-26 | m-D12 | H | H |
| 3552 | 0 | m-D12 | H | A-27 | m-D12 | H | H |
| 3553 | 0 | m-D12 | H | A-28 | m-D12 | H | H |
| 3554 | 0 | m-D12 | H | A-29 | m-D12 | H | H |
| 3555 | 0 | m-D12 | H | A-30 | m-D12 | H | H |
| 3556 | 0 | m-D12 | H | A-31 | m-D12 | H | H |
| 3557 | 0 | m-D12 | H | A-32 | m-D12 | H | H |
| 3558 | 0 | m-D12 | H | A-33 | m-D12 | H | H |
| 3559 | 0 | m-D12 | H | A-34 | m-D12 | H | H |
| 3560 | 0 | m-D12 | H | A-35 | m-D12 | H | H |
| 3561 | 0 | m-D12 | H | A-36 | m-D12 | H | H |
| 3562 | 0 | m-D12 | H | A-37 | m-D12 | H | H |
| 3563 | 0 | m-D12 | H | A-38 | m-D12 | H | H |
| 3564 | 0 | m-D12 | H | A-39 | m-D12 | H | H |
| 3565 | 0 | m-D12 | H | A-40 | m-D12 | H | H |
| 3566 | 0 | m-D12 | H | A-41 | m-D12 | H | H |
| 3567 | 0 | m-D12 | H | A-42 | m-D12 | H | H |
| 3568 | 0 | m-D12 | H | A-43 | m-D12 | H | H |
| 3569 | 0 | m-D12 | H | A-44 | m-D12 | H | H |
| 3570 | 0 | m-D12 | H | A-45 | m-D12 | H | H |
| 3571 | 0 | m-D12 | H | A-46 | m-D12 | H | H |
| 3572 | 0 | m-D12 | H | A-47 | m-D12 | H | H |
| 3573 | 0 | m-D13 | H | A-1 | m-D13 | H | H |
| 3574 | 0 | m-D13 | H | A-2 | m-D13 | H | H |
| 3575 | 0 | m-D13 | H | A-3 | m-D13 | H | H |
| 3576 | 0 | m-D13 | H | A-4 | m-D13 | H | H |
| 3577 | 0 | m-D13 | H | A-5 | m-D13 | H | H |
| 3578 | 0 | m-D13 | H | A-6 | m-D13 | H | H |
| 3579 | 0 | m-D13 | H | A-7 | m-D13 | H | H |
| 3580 | 0 | m-D13 | H | A-8 | m-D13 | H | H |
| 3581 | 0 | m-D13 | H | A-9 | m-D13 | H | H |
| 3582 | 0 | m-D13 | H | A-10 | m-D13 | H | H |
| 3583 | 0 | m-D13 | H | A-11 | m-D13 | H | H |
| 3584 | 0 | m-D13 | H | A-12 | m-D13 | H | H |
| 3585 | 0 | m-D13 | H | A-13 | m-D13 | H | H |
| 3586 | 0 | m-D13 | H | A-14 | m-D13 | H | H |
| 3587 | 0 | m-D13 | H | A-15 | m-D13 | H | H |
| 3588 | 0 | m-D13 | H | A-16 | m-D13 | H | H |
| 3589 | 0 | m-D13 | H | A-17 | m-D13 | H | H |
| 3590 | 0 | m-D13 | H | A-18 | m-D13 | H | H |
| 3591 | 0 | m-D13 | H | A-19 | m-D13 | H | H |
| 3592 | 0 | m-D13 | H | A-20 | m-D13 | H | H |
| 3593 | 0 | m-D13 | H | A-21 | m-D13 | H | H |
| 3594 | 0 | m-D13 | H | A-22 | m-D13 | H | H |
| 3595 | 0 | m-D13 | H | A-23 | m-D13 | H | H |
| 3596 | 0 | m-D13 | H | A-24 | m-D13 | H | H |
| 3597 | 0 | m-D13 | H | A-25 | m-D13 | H | H |
| 3598 | 0 | m-D13 | H | A-26 | m-D13 | H | H |
| 3599 | 0 | m-D13 | H | A-27 | m-D13 | H | H |
| 3600 | 0 | m-D13 | H | A-28 | m-D13 | H | H |
| 3601 | 0 | m-D13 | H | A-29 | m-D13 | H | H |
| 3602 | 0 | m-D13 | H | A-30 | m-D13 | H | H |
| 3603 | 0 | m-D13 | H | A-31 | m-D13 | H | H |
| 3604 | 0 | m-D13 | H | A-32 | m-D13 | H | H |
| 3605 | 0 | m-D13 | H | A-33 | m-D13 | H | H |
| 3606 | 0 | m-D13 | H | A-34 | m-D13 | H | H |
| 3607 | 0 | m-D13 | H | A-35 | m-D13 | H | H |
| 3608 | 0 | m-D13 | H | A-36 | m-D13 | H | H |
| 3609 | 0 | m-D13 | H | A-37 | m-D13 | H | H |
| 3610 | 0 | m-D13 | H | A-38 | m-D13 | H | H |
| 3611 | 0 | m-D13 | H | A-39 | m-D13 | H | H |
| 3612 | 0 | m-D13 | H | A-40 | m-D13 | H | H |
| 3613 | 0 | m-D13 | H | A-41 | m-D13 | H | H |
| 3614 | 0 | m-D13 | H | A-42 | m-D13 | H | H |
| 3615 | 0 | m-D13 | H | A-43 | m-D13 | H | H |
| 3616 | 0 | m-D13 | H | A-44 | m-D13 | H | H |
| 3617 | 0 | m-D13 | H | A-45 | m-D13 | H | H |
| 3618 | 0 | m-D13 | H | A-46 | m-D13 | H | H |
| 3619 | 0 | m-D13 | H | A-47 | m-D13 | H | H |
| 3620 | 0 | m-D14 | H | A-1 | m-D14 | H | H |
| 3621 | 0 | m-D14 | H | A-2 | m-D14 | H | H |
| 3622 | 0 | m-D14 | H | A-3 | m-D14 | H | H |
| 3623 | 0 | m-D14 | H | A-4 | m-D14 | H | H |
| 3624 | 0 | m-D14 | H | A-5 | m-D14 | H | H |
| 3625 | 0 | m-D14 | H | A-6 | m-D14 | H | H |
| 3626 | 0 | m-D14 | H | A-7 | m-D14 | H | H |
| 3627 | 0 | m-D14 | H | A-8 | m-D14 | H | H |
| 3628 | 0 | m-D14 | H | A-9 | m-D14 | H | H |
| 3629 | 0 | m-D14 | H | A-10 | m-D14 | H | H |
| 3630 | 0 | m-D14 | H | A-11 | m-D14 | H | H |
| 3631 | 0 | m-D14 | H | A-12 | m-D14 | H | H |
| 3632 | 0 | m-D14 | H | A-13 | m-D14 | H | H |
| 3633 | 0 | m-D14 | H | A-14 | m-D14 | H | H |
| 3634 | 0 | m-D14 | H | A-15 | m-D14 | H | H |
| 3635 | 0 | m-D14 | H | A-16 | m-D14 | H | H |
| 3636 | 0 | m-D14 | H | A-17 | m-D14 | H | H |
| 3637 | 0 | m-D14 | H | A-18 | m-D14 | H | H |
| 3638 | 0 | m-D14 | H | A-19 | m-D14 | H | H |
| 3639 | 0 | m-D14 | H | A-20 | m-D14 | H | H |
| 3640 | 0 | m-D14 | H | A-21 | m-D14 | H | H |
| 3641 | 0 | m-D14 | H | A-22 | m-D14 | H | H |
| 3642 | 0 | m-D14 | H | A-23 | m-D14 | H | H |
| 3643 | 0 | m-D14 | H | A-24 | m-D14 | H | H |
| 3644 | 0 | m-D14 | H | A-25 | m-D14 | H | H |
| 3645 | 0 | m-D14 | H | A-26 | m-D14 | H | H |
| 3646 | 0 | m-D14 | H | A-27 | m-D14 | H | H |
| 3647 | 0 | m-D14 | H | A-28 | m-D14 | H | H |
| 3648 | 0 | m-D14 | H | A-29 | m-D14 | H | H |
| 3649 | 0 | m-D14 | H | A-30 | m-D14 | H | H |
| 3650 | 0 | m-D14 | H | A-31 | m-D14 | H | H |
| 3651 | 0 | m-D14 | H | A-32 | m-D14 | H | H |
| 3652 | 0 | m-D14 | H | A-33 | m-D14 | H | H |
| 3653 | 0 | m-D14 | H | A-34 | m-D14 | H | H |
| 3654 | 0 | m-D14 | H | A-35 | m-D14 | H | H |
| 3655 | 0 | m-D14 | H | A-36 | m-D14 | H | H |
| 3656 | 0 | m-D14 | H | A-37 | m-D14 | H | H |
| 3657 | 0 | m-D14 | H | A-38 | m-D14 | H | H |
| 3658 | 0 | m-D14 | H | A-39 | m-D14 | H | H |
| 3659 | 0 | m-D14 | H | A-40 | m-D14 | H | H |
| 3660 | 0 | m-D14 | H | A-41 | m-D14 | H | H |
| 3661 | 0 | m-D14 | H | A-42 | m-D14 | H | H |
| 3662 | 0 | m-D14 | H | A-43 | m-D14 | H | H |
| 3663 | 0 | m-D14 | H | A-44 | m-D14 | H | H |
| 3664 | 0 | m-D14 | H | A-45 | m-D14 | H | H |
| 3665 | 0 | m-D14 | H | A-46 | m-D14 | H | H |
| 3666 | 0 | m-D14 | H | A-47 | m-D14 | H | H |
| 3667 | 0 | m-D1 | H | m-D1 | A-1 | H | H |
| 3668 | 0 | m-D1 | H | m-D1 | A-2 | H | H |
| 3669 | 0 | m-D1 | H | m-D1 | A-3 | H | H |
| 3670 | 0 | m-D1 | H | m-D1 | A-4 | H | H |
| 3671 | 0 | m-D1 | H | m-D1 | A-5 | H | H |
| 3672 | 0 | m-D1 | H | m-D1 | A-6 | H | H |
| 3673 | 0 | m-D1 | H | m-D1 | A-7 | H | H |
| 3674 | 0 | m-D1 | H | m-D1 | A-8 | H | H |
| 3675 | 0 | m-D1 | H | m-D1 | A-9 | H | H |
| 3676 | 0 | m-D1 | H | m-D1 | A-10 | H | H |
| 3677 | 0 | m-D1 | H | m-D1 | A-11 | H | H |
| 3678 | 0 | m-D1 | H | m-D1 | A-12 | H | H |
| 3679 | 0 | m-D1 | H | m-D1 | A-13 | H | H |
| 3680 | 0 | m-D1 | H | m-D1 | A-14 | H | H |
| 3681 | 0 | m-D1 | H | m-D1 | A-15 | H | H |
| 3682 | 0 | m-D1 | H | m-D1 | A-16 | H | H |
| 3683 | 0 | m-D1 | H | m-D1 | A-17 | H | H |
| 3684 | 0 | m-D1 | H | m-D1 | A-18 | H | H |
| 3685 | 0 | m-D1 | H | m-D1 | A-19 | H | H |
| 3686 | 0 | m-D1 | H | m-D1 | A-20 | H | H |
| 3687 | 0 | m-D1 | H | m-D1 | A-21 | H | H |
| 3688 | 0 | m-D1 | H | m-D1 | A-22 | H | H |
| 3689 | 0 | m-D1 | H | m-D1 | A-23 | H | H |
| 3690 | 0 | m-D1 | H | m-D1 | A-24 | H | H |
| 3691 | 0 | m-D1 | H | m-D1 | A-25 | H | H |
| 3692 | 0 | m-D1 | H | m-D1 | A-26 | H | H |
| 3693 | 0 | m-D1 | H | m-D1 | A-27 | H | H |
| 3694 | 0 | m-D1 | H | m-D1 | A-28 | H | H |
| 3695 | 0 | m-D1 | H | m-D1 | A-29 | H | H |
| 3696 | 0 | m-D1 | H | m-D1 | A-30 | H | H |
| 3697 | 0 | m-D1 | H | m-D1 | A-31 | H | H |
| 3698 | 0 | m-D1 | H | m-D1 | A-32 | H | H |
| 3699 | 0 | m-D1 | H | m-D1 | A-33 | H | H |
| 3700 | 0 | m-D1 | H | m-D1 | A-34 | H | H |
| 3701 | 0 | m-D1 | H | m-D1 | A-35 | H | H |
| 3702 | 0 | m-D1 | H | m-D1 | A-36 | H | H |
| 3703 | 0 | m-D1 | H | m-D1 | A-37 | H | H |
| 3704 | 0 | m-D1 | H | m-D1 | A-38 | H | H |
| 3705 | 0 | m-D1 | H | m-D1 | A-39 | H | H |
| 3706 | 0 | m-D1 | H | m-D1 | A-40 | H | H |
| 3707 | 0 | m-D1 | H | m-D1 | A-41 | H | H |
| 3708 | 0 | m-D1 | H | m-D1 | A-42 | H | H |
| 3709 | 0 | m-D1 | H | m-D1 | A-43 | H | H |
| 3710 | 0 | m-D1 | H | m-D1 | A-44 | H | H |
| 3711 | 0 | m-D1 | H | m-D1 | A-45 | H | H |
| 3712 | 0 | m-D1 | H | m-D1 | A-46 | H | H |
| 3713 | 0 | m-D1 | H | m-D1 | A-47 | H | H |
| 3714 | 0 | m-D2 | H | m-D2 | A-1 | H | H |
| 3715 | 0 | m-D2 | H | m-D2 | A-2 | H | H |
| 3716 | 0 | m-D2 | H | m-D2 | A-3 | H | H |
| 3717 | 0 | m-D2 | H | m-D2 | A-4 | H | H |
| 3718 | 0 | m-D2 | H | m-D2 | A-5 | H | H |
| 3719 | 0 | m-D2 | H | m-D2 | A-6 | H | H |
| 3720 | 0 | m-D2 | H | m-D2 | A-7 | H | H |
| 3721 | 0 | m-D2 | H | m-D2 | A-8 | H | H |
| 3722 | 0 | m-D2 | H | m-D2 | A-9 | H | H |
| 3723 | 0 | m-D2 | H | m-D2 | A-10 | H | H |
| 3724 | 0 | m-D2 | H | m-D2 | A-11 | H | H |
| 3725 | 0 | m-D2 | H | m-D2 | A-12 | H | H |
| 3726 | 0 | m-D2 | H | m-D2 | A-13 | H | H |
| 3727 | 0 | m-D2 | H | m-D2 | A-14 | H | H |
| 3728 | 0 | m-D2 | H | m-D2 | A-15 | H | H |
| 3729 | 0 | m-D2 | H | m-D2 | A-16 | H | H |
| 3730 | 0 | m-D2 | H | m-D2 | A-17 | H | H |
| 3731 | 0 | m-D2 | H | m-D2 | A-18 | H | H |
| 3732 | 0 | m-D2 | H | m-D2 | A-19 | H | H |
| 3733 | 0 | m-D2 | H | m-D2 | A-20 | H | H |
| 3734 | 0 | m-D2 | H | m-D2 | A-21 | H | H |
| 3735 | 0 | m-D2 | H | m-D2 | A-22 | H | H |
| 3736 | 0 | m-D2 | H | m-D2 | A-23 | H | H |
| 3737 | 0 | m-D2 | H | m-D2 | A-24 | H | H |
| 3738 | 0 | m-D2 | H | m-D2 | A-25 | H | H |
| 3739 | 0 | m-D2 | H | m-D2 | A-26 | H | H |
| 3740 | 0 | m-D2 | H | m-D2 | A-27 | H | H |
| 3741 | 0 | m-D2 | H | m-D2 | A-28 | H | H |
| 3742 | 0 | m-D2 | H | m-D2 | A-29 | H | H |
| 3743 | 0 | m-D2 | H | m-D2 | A-30 | H | H |
| 3744 | 0 | m-D2 | H | m-D2 | A-31 | H | H |
| 3745 | 0 | m-D2 | H | m-D2 | A-32 | H | H |
| 3746 | 0 | m-D2 | H | m-D2 | A-33 | H | H |
| 3747 | 0 | m-D2 | H | m-D2 | A-34 | H | H |
| 3748 | 0 | m-D2 | H | m-D2 | A-35 | H | H |
| 3749 | 0 | m-D2 | H | m-D2 | A-36 | H | H |
| 3750 | 0 | m-D2 | H | m-D2 | A-37 | H | H |
| 3751 | 0 | m-D2 | H | m-D2 | A-38 | H | H |
| 3752 | 0 | m-D2 | H | m-D2 | A-39 | H | H |
| 3753 | 0 | m-D2 | H | m-D2 | A-40 | H | H |
| 3754 | 0 | m-D2 | H | m-D2 | A-41 | H | H |
| 3755 | 0 | m-D2 | H | m-D2 | A-42 | H | H |
| 3756 | 0 | m-D2 | H | m-D2 | A-43 | H | H |
| 3757 | 0 | m-D2 | H | m-D2 | A-44 | H | H |
| 3758 | 0 | m-D2 | H | m-D2 | A-45 | H | H |
| 3759 | 0 | m-D2 | H | m-D2 | A-46 | H | H |
| 3760 | 0 | m-D2 | H | m-D2 | A-47 | H | H |
| 3761 | 0 | m-D3 | H | m-D3 | A-1 | H | H |
| 3762 | 0 | m-D3 | H | m-D3 | A-2 | H | H |
| 3763 | 0 | m-D3 | H | m-D3 | A-3 | H | H |
| 3764 | 0 | m-D3 | H | m-D3 | A-4 | H | H |
| 3765 | 0 | m-D3 | H | m-D3 | A-5 | H | H |
| 3766 | 0 | m-D3 | H | m-D3 | A-6 | H | H |
| 3767 | 0 | m-D3 | H | m-D3 | A-7 | H | H |
| 3768 | 0 | m-D3 | H | m-D3 | A-8 | H | H |
| 3769 | 0 | m-D3 | H | m-D3 | A-9 | H | H |
| 3770 | 0 | m-D3 | H | m-D3 | A-10 | H | H |
| 3771 | 0 | m-D3 | H | m-D3 | A-11 | H | H |
| 3772 | 0 | m-D3 | H | m-D3 | A-12 | H | H |
| 3773 | 0 | m-D3 | H | m-D3 | A-13 | H | H |
| 3774 | 0 | m-D3 | H | m-D3 | A-14 | H | H |
| 3775 | 0 | m-D3 | H | m-D3 | A-15 | H | H |
| 3776 | 0 | m-D3 | H | m-D3 | A-16 | H | H |
| 3777 | 0 | m-D3 | H | m-D3 | A-17 | H | H |
| 3778 | 0 | m-D3 | H | m-D3 | A-18 | H | H |
| 3779 | 0 | m-D3 | H | m-D3 | A-19 | H | H |
| 3780 | 0 | m-D3 | H | m-D3 | A-20 | H | H |
| 3781 | 0 | m-D3 | H | m-D3 | A-21 | H | H |
| 3782 | 0 | m-D3 | H | m-D3 | A-22 | H | H |
| 3783 | 0 | m-D3 | H | m-D3 | A-23 | H | H |
| 3784 | 0 | m-D3 | H | m-D3 | A-24 | H | H |
| 3785 | 0 | m-D3 | H | m-D3 | A-25 | H | H |
| 3786 | 0 | m-D3 | H | m-D3 | A-26 | H | H |
| 3787 | 0 | m-D3 | H | m-D3 | A-27 | H | H |
| 3788 | 0 | m-D3 | H | m-D3 | A-28 | H | H |
| 3789 | 0 | m-D3 | H | m-D3 | A-29 | H | H |
| 3790 | 0 | m-D3 | H | m-D3 | A-30 | H | H |
| 3791 | 0 | m-D3 | H | m-D3 | A-31 | H | H |
| 3792 | 0 | m-D3 | H | m-D3 | A-32 | H | H |
| 3793 | 0 | m-D3 | H | m-D3 | A-33 | H | H |
| 3794 | 0 | m-D3 | H | m-D3 | A-34 | H | H |
| 3795 | 0 | m-D3 | H | m-D3 | A-35 | H | H |
| 3796 | 0 | m-D3 | H | m-D3 | A-36 | H | H |
| 3797 | 0 | m-D3 | H | m-D3 | A-37 | H | H |
| 3798 | 0 | m-D3 | H | m-D3 | A-38 | H | H |
| 3799 | 0 | m-D3 | H | m-D3 | A-39 | H | H |
| 3800 | 0 | m-D3 | H | m-D3 | A-40 | H | H |
| 3801 | 0 | m-D3 | H | m-D3 | A-41 | H | H |
| 3802 | 0 | m-D3 | H | m-D3 | A-42 | H | H |
| 3803 | 0 | m-D3 | H | m-D3 | A-43 | H | H |
| 3804 | 0 | m-D3 | H | m-D3 | A-44 | H | H |
| 3805 | 0 | m-D3 | H | m-D3 | A-45 | H | H |
| 3806 | 0 | m-D3 | H | m-D3 | A-46 | H | H |
| 3807 | 0 | m-D3 | H | m-D3 | A-47 | H | H |
| 3808 | 0 | m-D5 | H | m-D5 | A-1 | H | H |
| 3809 | 0 | m-D5 | H | m-D5 | A-2 | H | H |
| 3810 | 0 | m-D5 | H | m-D5 | A-3 | H | H |
| 3811 | 0 | m-D5 | H | m-D5 | A-4 | H | H |
| 3812 | 0 | m-D5 | H | m-D5 | A-5 | H | H |
| 3813 | 0 | m-D5 | H | m-D5 | A-6 | H | H |
| 3814 | 0 | m-D5 | H | m-D5 | A-7 | H | H |
| 3815 | 0 | m-D5 | H | m-D5 | A-8 | H | H |
| 3816 | 0 | m-D5 | H | m-D5 | A-9 | H | H |
| 3817 | 0 | m-D5 | H | m-D5 | A-10 | H | H |
| 3818 | 0 | m-D5 | H | m-D5 | A-11 | H | H |
| 3819 | 0 | m-D5 | H | m-D5 | A-12 | H | H |
| 3820 | 0 | m-D5 | H | m-D5 | A-13 | H | H |
| 3821 | 0 | m-D5 | H | m-D5 | A-14 | H | H |
| 3822 | 0 | m-D5 | H | m-D5 | A-15 | H | H |
| 3823 | 0 | m-D5 | H | m-D5 | A-16 | H | H |
| 3824 | 0 | m-D5 | H | m-D5 | A-17 | H | H |
| 3825 | 0 | m-D5 | H | m-D5 | A-18 | H | H |
| 3826 | 0 | m-D5 | H | m-D5 | A-19 | H | H |
| 3827 | 0 | m-D5 | H | m-D5 | A-20 | H | H |
| 3828 | 0 | m-D5 | H | m-D5 | A-21 | H | H |
| 3829 | 0 | m-D5 | H | m-D5 | A-22 | H | H |
| 3830 | 0 | m-D5 | H | m-D5 | A-23 | H | H |
| 3831 | 0 | m-D5 | H | m-D5 | A-24 | H | H |
| 3832 | 0 | m-D5 | H | m-D5 | A-25 | H | H |
| 3833 | 0 | m-D5 | H | m-D5 | A-26 | H | H |
| 3834 | 0 | m-D5 | H | m-D5 | A-27 | H | H |
| 3835 | 0 | m-D5 | H | m-D5 | A-28 | H | H |
| 3836 | 0 | m-D5 | H | m-D5 | A-29 | H | H |
| 3837 | 0 | m-D5 | H | m-D5 | A-30 | H | H |
| 3838 | 0 | m-D5 | H | m-D5 | A-31 | H | H |
| 3839 | 0 | m-D5 | H | m-D5 | A-32 | H | H |
| 3840 | 0 | m-D5 | H | m-D5 | A-33 | H | H |
| 3841 | 0 | m-D5 | H | m-D5 | A-34 | H | H |
| 3842 | 0 | m-D5 | H | m-D5 | A-35 | H | H |
| 3843 | 0 | m-D5 | H | m-D5 | A-36 | H | H |
| 3844 | 0 | m-D5 | H | m-D5 | A-37 | H | H |
| 3845 | 0 | m-D5 | H | m-D5 | A-38 | H | H |
| 3846 | 0 | m-D5 | H | m-D5 | A-39 | H | H |
| 3847 | 0 | m-D5 | H | m-D5 | A-40 | H | H |
| 3848 | 0 | m-D5 | H | m-D5 | A-41 | H | H |
| 3849 | 0 | m-D5 | H | m-D5 | A-42 | H | H |
| 3850 | 0 | m-D5 | H | m-D5 | A-43 | H | H |
| 3851 | 0 | m-D5 | H | m-D5 | A-44 | H | H |
| 3852 | 0 | m-D5 | H | m-D5 | A-45 | H | H |
| 3853 | 0 | m-D5 | H | m-D5 | A-46 | H | H |
| 3854 | 0 | m-D5 | H | m-D5 | A-47 | H | H |
| 3855 | 0 | m-D9 | H | m-D9 | A-1 | H | H |
| 3856 | 0 | m-D9 | H | m-D9 | A-2 | H | H |
| 3857 | 0 | m-D9 | H | m-D9 | A-3 | H | H |
| 3858 | 0 | m-D9 | H | m-D9 | A-4 | H | H |
| 3859 | 0 | m-D9 | H | m-D9 | A-5 | H | H |
| 3860 | 0 | m-D9 | H | m-D9 | A-6 | H | H |
| 3861 | 0 | m-D9 | H | m-D9 | A-7 | H | H |
| 3862 | 0 | m-D9 | H | m-D9 | A-8 | H | H |
| 3863 | 0 | m-D9 | H | m-D9 | A-9 | H | H |
| 3864 | 0 | m-D9 | H | m-D9 | A-10 | H | H |
| 3865 | 0 | m-D9 | H | m-D9 | A-11 | H | H |
| 3866 | 0 | m-D9 | H | m-D9 | A-12 | H | H |
| 3867 | 0 | m-D9 | H | m-D9 | A-13 | H | H |
| 3868 | 0 | m-D9 | H | m-D9 | A-14 | H | H |
| 3869 | 0 | m-D9 | H | m-D9 | A-15 | H | H |
| 3870 | 0 | m-D9 | H | m-D9 | A-16 | H | H |
| 3871 | 0 | m-D9 | H | m-D9 | A-17 | H | H |
| 3872 | 0 | m-D9 | H | m-D9 | A-18 | H | H |
| 3873 | 0 | m-D9 | H | m-D9 | A-19 | H | H |
| 3874 | 0 | m-D9 | H | m-D9 | A-20 | H | H |
| 3875 | 0 | m-D9 | H | m-D9 | A-21 | H | H |
| 3876 | 0 | m-D9 | H | m-D9 | A-22 | H | H |
| 3877 | 0 | m-D9 | H | m-D9 | A-23 | H | H |
| 3878 | 0 | m-D9 | H | m-D9 | A-24 | H | H |
| 3879 | 0 | m-D9 | H | m-D9 | A-25 | H | H |
| 3880 | 0 | m-D9 | H | m-D9 | A-26 | H | H |
| 3881 | 0 | m-D9 | H | m-D9 | A-27 | H | H |
| 3882 | 0 | m-D9 | H | m-D9 | A-28 | H | H |
| 3883 | 0 | m-D9 | H | m-D9 | A-29 | H | H |
| 3884 | 0 | m-D9 | H | m-D9 | A-30 | H | H |
| 3885 | 0 | m-D9 | H | m-D9 | A-31 | H | H |
| 3886 | 0 | m-D9 | H | m-D9 | A-32 | H | H |
| 3887 | 0 | m-D9 | H | m-D9 | A-33 | H | H |
| 3888 | 0 | m-D9 | H | m-D9 | A-34 | H | H |
| 3889 | 0 | m-D9 | H | m-D9 | A-35 | H | H |
| 3890 | 0 | m-D9 | H | m-D9 | A-36 | H | H |
| 3891 | 0 | m-D9 | H | m-D9 | A-37 | H | H |
| 3892 | 0 | m-D9 | H | m-D9 | A-38 | H | H |
| 3893 | 0 | m-D9 | H | m-D9 | A-39 | H | H |
| 3894 | 0 | m-D9 | H | m-D9 | A-40 | H | H |
| 3895 | 0 | m-D9 | H | m-D9 | A-41 | H | H |
| 3896 | 0 | m-D9 | H | m-D9 | A-42 | H | H |
| 3897 | 0 | m-D9 | H | m-D9 | A-43 | H | H |
| 3898 | 0 | m-D9 | H | m-D9 | A-44 | H | H |
| 3899 | 0 | m-D9 | H | m-D9 | A-45 | H | H |
| 3900 | 0 | m-D9 | H | m-D9 | A-46 | H | H |
| 3901 | 0 | m-D9 | H | m-D9 | A-47 | H | H |
| 3902 | 0 | m-D11 | H | m-D11 | A-1 | H | H |
| 3903 | 0 | m-D11 | H | m-D11 | A-2 | H | H |
| 3904 | 0 | m-D11 | H | m-D11 | A-3 | H | H |
| 3905 | 0 | m-D11 | H | m-D11 | A-4 | H | H |
| 3906 | 0 | m-D11 | H | m-D11 | A-5 | H | H |
| 3907 | 0 | m-D11 | H | m-D11 | A-6 | H | H |
| 3908 | 0 | m-D11 | H | m-D11 | A-7 | H | H |
| 3909 | 0 | m-D11 | H | m-D11 | A-8 | H | H |
| 3910 | 0 | m-D11 | H | m-D11 | A-9 | H | H |
| 3911 | 0 | m-D11 | H | m-D11 | A-10 | H | H |
| 3912 | 0 | m-D11 | H | m-D11 | A-11 | H | H |
| 3913 | 0 | m-D11 | H | m-D11 | A-12 | H | H |
| 3914 | 0 | m-D11 | H | m-D11 | A-13 | H | H |
| 3915 | 0 | m-D11 | H | m-D11 | A-14 | H | H |
| 3916 | 0 | m-D11 | H | m-D11 | A-15 | H | H |
| 3917 | 0 | m-D11 | H | m-D11 | A-16 | H | H |
| 3918 | 0 | m-D11 | H | m-D11 | A-17 | H | H |
| 3919 | 0 | m-D11 | H | m-D11 | A-18 | H | H |
| 3920 | 0 | m-D11 | H | m-D11 | A-19 | H | H |
| 3921 | 0 | m-D11 | H | m-D11 | A-20 | H | H |
| 3922 | 0 | m-D11 | H | m-D11 | A-21 | H | H |
| 3923 | 0 | m-D11 | H | m-D11 | A-22 | H | H |
| 3924 | 0 | m-D11 | H | m-D11 | A-23 | H | H |
| 3925 | 0 | m-D11 | H | m-D11 | A-24 | H | H |
| 3926 | 0 | m-D11 | H | m-D11 | A-25 | H | H |
| 3927 | 0 | m-D11 | H | m-D11 | A-26 | H | H |
| 3928 | 0 | m-D11 | H | m-D11 | A-27 | H | H |
| 3929 | 0 | m-D11 | H | m-D11 | A-28 | H | H |
| 3930 | 0 | m-D11 | H | m-D11 | A-29 | H | H |
| 3931 | 0 | m-D11 | H | m-D11 | A-30 | H | H |
| 3932 | 0 | m-D11 | H | m-D11 | A-31 | H | H |
| 3933 | 0 | m-D11 | H | m-D11 | A-32 | H | H |
| 3934 | 0 | m-D11 | H | m-D11 | A-33 | H | H |
| 3935 | 0 | m-D11 | H | m-D11 | A-34 | H | H |
| 3936 | 0 | m-D11 | H | m-D11 | A-35 | H | H |
| 3937 | 0 | m-D11 | H | m-D11 | A-36 | H | H |
| 3938 | 0 | m-D11 | H | m-D11 | A-37 | H | H |
| 3939 | 0 | m-D11 | H | m-D11 | A-38 | H | H |
| 3940 | 0 | m-D11 | H | m-D11 | A-39 | H | H |
| 3941 | 0 | m-D11 | H | m-D11 | A-40 | H | H |
| 3942 | 0 | m-D11 | H | m-D11 | A-41 | H | H |
| 3943 | 0 | m-D11 | H | m-D11 | A-42 | H | H |
| 3944 | 0 | m-D11 | H | m-D11 | A-43 | H | H |
| 3945 | 0 | m-D11 | H | m-D11 | A-44 | H | H |
| 3946 | 0 | m-D11 | H | m-D11 | A-45 | H | H |
| 3947 | 0 | m-D11 | H | m-D11 | A-46 | H | H |
| 3948 | 0 | m-D11 | H | m-D11 | A-47 | H | H |
| 3949 | 0 | m-D12 | H | m-D12 | A-1 | H | H |
| 3950 | 0 | m-D12 | H | m-D12 | A-2 | H | H |
| 3951 | 0 | m-D12 | H | m-D12 | A-3 | H | H |
| 3952 | 0 | m-D12 | H | m-D12 | A-4 | H | H |
| 3953 | 0 | m-D12 | H | m-D12 | A-5 | H | H |
| 3954 | 0 | m-D12 | H | m-D12 | A-6 | H | H |
| 3955 | 0 | m-D12 | H | m-D12 | A-7 | H | H |
| 3956 | 0 | m-D12 | H | m-D12 | A-8 | H | H |
| 3957 | 0 | m-D12 | H | m-D12 | A-9 | H | H |
| 3958 | 0 | m-D12 | H | m-D12 | A-10 | H | H |
| 3959 | 0 | m-D12 | H | m-D12 | A-11 | H | H |
| 3960 | 0 | m-D12 | H | m-D12 | A-12 | H | H |
| 3961 | 0 | m-D12 | H | m-D12 | A-13 | H | H |
| 3962 | 0 | m-D12 | H | m-D12 | A-14 | H | H |
| 3963 | 0 | m-D12 | H | m-D12 | A-15 | H | H |
| 3964 | 0 | m-D12 | H | m-D12 | A-16 | H | H |
| 3965 | 0 | m-D12 | H | m-D12 | A-17 | H | H |
| 3966 | 0 | m-D12 | H | m-D12 | A-18 | H | H |
| 3967 | 0 | m-D12 | H | m-D12 | A-19 | H | H |
| 3968 | 0 | m-D12 | H | m-D12 | A-20 | H | H |
| 3969 | 0 | m-D12 | H | m-D12 | A-21 | H | H |
| 3970 | 0 | m-D12 | H | m-D12 | A-22 | H | H |
| 3971 | 0 | m-D12 | H | m-D12 | A-23 | H | H |
| 3972 | 0 | m-D12 | H | m-D12 | A-24 | H | H |
| 3973 | 0 | m-D12 | H | m-D12 | A-25 | H | H |
| 3974 | 0 | m-D12 | H | m-D12 | A-26 | H | H |
| 3975 | 0 | m-D12 | H | m-D12 | A-27 | H | H |
| 3976 | 0 | m-D12 | H | m-D12 | A-28 | H | H |
| 3977 | 0 | m-D12 | H | m-D12 | A-29 | H | H |
| 3978 | 0 | m-D12 | H | m-D12 | A-30 | H | H |
| 3979 | 0 | m-D12 | H | m-D12 | A-31 | H | H |
| 3980 | 0 | m-D12 | H | m-D12 | A-32 | H | H |
| 3981 | 0 | m-D12 | H | m-D12 | A-33 | H | H |
| 3982 | 0 | m-D12 | H | m-D12 | A-34 | H | H |
| 3983 | 0 | m-D12 | H | m-D12 | A-35 | H | H |
| 3984 | 0 | m-D12 | H | m-D12 | A-36 | H | H |
| 3985 | 0 | m-D12 | H | m-D12 | A-37 | H | H |
| 3986 | 0 | m-D12 | H | m-D12 | A-38 | H | H |
| 3987 | 0 | m-D12 | H | m-D12 | A-39 | H | H |
| 3988 | 0 | m-D12 | H | m-D12 | A-40 | H | H |
| 3989 | 0 | m-D12 | H | m-D12 | A-41 | H | H |
| 3990 | 0 | m-D12 | H | m-D12 | A-42 | H | H |
| 3991 | 0 | m-D12 | H | m-D12 | A-43 | H | H |
| 3992 | 0 | m-D12 | H | m-D12 | A-44 | H | H |
| 3993 | 0 | m-D12 | H | m-D12 | A-45 | H | H |
| 3994 | 0 | m-D12 | H | m-D12 | A-46 | H | H |
| 3995 | 0 | m-D12 | H | m-D12 | A-47 | H | H |
| 3996 | 0 | m-D13 | H | m-D13 | A-1 | H | H |
| 3997 | 0 | m-D13 | H | m-D13 | A-2 | H | H |
| 3998 | 0 | m-D13 | H | m-D13 | A-3 | H | H |
| 3999 | 0 | m-D13 | H | m-D13 | A-4 | H | H |
| 4000 | 0 | m-D13 | H | m-D13 | A-5 | H | H |
| 4001 | 0 | m-D13 | H | m-D13 | A-6 | H | H |
| 4002 | 0 | m-D13 | H | m-D13 | A-7 | H | H |
| 4003 | 0 | m-D13 | H | m-D13 | A-8 | H | H |
| 4004 | 0 | m-D13 | H | m-D13 | A-9 | H | H |
| 4005 | 0 | m-D13 | H | m-D13 | A-10 | H | H |
| 4006 | 0 | m-D13 | H | m-D13 | A-11 | H | H |
| 4007 | 0 | m-D13 | H | m-D13 | A-12 | H | H |
| 4008 | 0 | m-D13 | H | m-D13 | A-13 | H | H |
| 4009 | 0 | m-D13 | H | m-D13 | A-14 | H | H |
| 4010 | 0 | m-D13 | H | m-D13 | A-15 | H | H |
| 4011 | 0 | m-D13 | H | m-D13 | A-16 | H | H |
| 4012 | 0 | m-D13 | H | m-D13 | A-17 | H | H |
| 4013 | 0 | m-D13 | H | m-D13 | A-18 | H | H |
| 4014 | 0 | m-D13 | H | m-D13 | A-19 | H | H |
| 4015 | 0 | m-D13 | H | m-D13 | A-20 | H | H |
| 4016 | 0 | m-D13 | H | m-D13 | A-21 | H | H |
| 4017 | 0 | m-D13 | H | m-D13 | A-22 | H | H |
| 4018 | 0 | m-D13 | H | m-D13 | A-23 | H | H |
| 4019 | 0 | m-D13 | H | m-D13 | A-24 | H | H |
| 4020 | 0 | m-D13 | H | m-D13 | A-25 | H | H |
| 4021 | 0 | m-D13 | H | m-D13 | A-26 | H | H |
| 4022 | 0 | m-D13 | H | m-D13 | A-27 | H | H |
| 4023 | 0 | m-D13 | H | m-D13 | A-28 | H | H |
| 4024 | 0 | m-D13 | H | m-D13 | A-29 | H | H |
| 4025 | 0 | m-D13 | H | m-D13 | A-30 | H | H |
| 4026 | 0 | m-D13 | H | m-D13 | A-31 | H | H |
| 4027 | 0 | m-D13 | H | m-D13 | A-32 | H | H |
| 4028 | 0 | m-D13 | H | m-D13 | A-33 | H | H |
| 4029 | 0 | m-D13 | H | m-D13 | A-34 | H | H |
| 4030 | 0 | m-D13 | H | m-D13 | A-35 | H | H |
| 4031 | 0 | m-D13 | H | m-D13 | A-36 | H | H |
| 4032 | 0 | m-D13 | H | m-D13 | A-37 | H | H |
| 4033 | 0 | m-D13 | H | m-D13 | A-38 | H | H |
| 4034 | 0 | m-D13 | H | m-D13 | A-39 | H | H |
| 4035 | 0 | m-D13 | H | m-D13 | A-40 | H | H |
| 4036 | 0 | m-D13 | H | m-D13 | A-41 | H | H |
| 4037 | 0 | m-D13 | H | m-D13 | A-42 | H | H |
| 4038 | 0 | m-D13 | H | m-D13 | A-43 | H | H |
| 4039 | 0 | m-D13 | H | m-D13 | A-44 | H | H |
| 4040 | 0 | m-D13 | H | m-D13 | A-45 | H | H |
| 4041 | 0 | m-D13 | H | m-D13 | A-46 | H | H |
| 4042 | 0 | m-D13 | H | m-D13 | A-47 | H | H |
| 4043 | 0 | m-D14 | H | m-D14 | A-1 | H | H |
| 4044 | 0 | m-D14 | H | m-D14 | A-2 | H | H |
| 4045 | 0 | m-D14 | H | m-D14 | A-3 | H | H |
| 4046 | 0 | m-D14 | H | m-D14 | A-4 | H | H |
| 4047 | 0 | m-D14 | H | m-D14 | A-5 | H | H |
| 4048 | 0 | m-D14 | H | m-D14 | A-6 | H | H |
| 4049 | 0 | m-D14 | H | m-D14 | A-7 | H | H |
| 4050 | 0 | m-D14 | H | m-D14 | A-8 | H | H |
| 4051 | 0 | m-D14 | H | m-D14 | A-9 | H | H |
| 4052 | 0 | m-D14 | H | m-D14 | A-10 | H | H |
| 4053 | 0 | m-D14 | H | m-D14 | A-11 | H | H |
| 4054 | 0 | m-D14 | H | m-D14 | A-12 | H | H |
| 4055 | 0 | m-D14 | H | m-D14 | A-13 | H | H |
| 4056 | 0 | m-D14 | H | m-D14 | A-14 | H | H |
| 4057 | 0 | m-D14 | H | m-D14 | A-15 | H | H |
| 4058 | 0 | m-D14 | H | m-D14 | A-16 | H | H |
| 4059 | 0 | m-D14 | H | m-D14 | A-17 | H | H |
| 4060 | 0 | m-D14 | H | m-D14 | A-18 | H | H |
| 4061 | 0 | m-D14 | H | m-D14 | A-19 | H | H |
| 4062 | 0 | m-D14 | H | m-D14 | A-20 | H | H |
| 4063 | 0 | m-D14 | H | m-D14 | A-21 | H | H |
| 4064 | 0 | m-D14 | H | m-D14 | A-22 | H | H |
| 4065 | 0 | m-D14 | H | m-D14 | A-23 | H | H |
| 4066 | 0 | m-D14 | H | m-D14 | A-24 | H | H |
| 4067 | 0 | m-D14 | H | m-D14 | A-25 | H | H |
| 4068 | 0 | m-D14 | H | m-D14 | A-26 | H | H |
| 4069 | 0 | m-D14 | H | m-D14 | A-27 | H | H |
| 4070 | 0 | m-D14 | H | m-D14 | A-28 | H | H |
| 4071 | 0 | m-D14 | H | m-D14 | A-29 | H | H |
| 4072 | 0 | m-D14 | H | m-D14 | A-30 | H | H |
| 4073 | 0 | m-D14 | H | m-D14 | A-31 | H | H |
| 4074 | 0 | m-D14 | H | m-D14 | A-32 | H | H |
| 4075 | 0 | m-D14 | H | m-D14 | A-33 | H | H |
| 4076 | 0 | m-D14 | H | m-D14 | A-34 | H | H |
| 4077 | 0 | m-D14 | H | m-D14 | A-35 | H | H |
| 4078 | 0 | m-D14 | H | m-D14 | A-36 | H | H |
| 4079 | 0 | m-D14 | H | m-D14 | A-37 | H | H |
| 4080 | 0 | m-D14 | H | m-D14 | A-38 | H | H |
| 4081 | 0 | m-D14 | H | m-D14 | A-39 | H | H |
| 4082 | 0 | m-D14 | H | m-D14 | A-40 | H | H |
| 4083 | 0 | m-D14 | H | m-D14 | A-41 | H | H |
| 4084 | 0 | m-D14 | H | m-D14 | A-42 | H | H |
| 4085 | 0 | m-D14 | H | m-D14 | A-43 | H | H |
| 4086 | 0 | m-D14 | H | m-D14 | A-44 | H | H |
| 4087 | 0 | m-D14 | H | m-D14 | A-45 | H | H |
| 4088 | 0 | m-D14 | H | m-D14 | A-46 | H | H |
| 4089 | 0 | m-D14 | H | m-D14 | A-47 | H | H |
| 4090 | 0 | A-1 | H | A-1 | m-D1 | H | H |
| 4091 | 0 | A-2 | H | A-2 | m-D1 | H | H |
| 4092 | 0 | A-3 | H | A-3 | m-D1 | H | H |
| 4093 | 0 | A-4 | H | A-4 | m-D1 | H | H |
| 4094 | 0 | A-5 | H | A-5 | m-D1 | H | H |
| 4095 | 0 | A-6 | H | A-6 | m-D1 | H | H |
| 4096 | 0 | A-7 | H | A-7 | m-D1 | H | H |
| 4097 | 0 | A-8 | H | A-8 | m-D1 | H | H |
| 4098 | 0 | A-9 | H | A-9 | m-D1 | H | H |
| 4099 | 0 | A-10 | H | A-10 | m-D1 | H | H |
| 4100 | 0 | A-11 | H | A-11 | m-D1 | H | H |
| 4101 | 0 | A-12 | H | A-12 | m-D1 | H | H |
| 4102 | 0 | A-13 | H | A-13 | m-D1 | H | H |
| 4103 | 0 | A-14 | H | A-14 | m-D1 | H | H |
| 4104 | 0 | A-15 | H | A-15 | m-D1 | H | H |
| 4105 | 0 | A-16 | H | A-16 | m-D1 | H | H |
| 4106 | 0 | A-17 | H | A-17 | m-D1 | H | H |
| 4107 | 0 | A-18 | H | A-18 | m-D1 | H | H |
| 4108 | 0 | A-19 | H | A-19 | m-D1 | H | H |
| 4109 | 0 | A-20 | H | A-20 | m-D1 | H | H |
| 4110 | 0 | A-21 | H | A-21 | m-D1 | H | H |
| 4111 | 0 | A-22 | H | A-22 | m-D1 | H | H |
| 4112 | 0 | A-23 | H | A-23 | m-D1 | H | H |
| 4113 | 0 | A-24 | H | A-24 | m-D1 | H | H |
| 4114 | 0 | A-25 | H | A-25 | m-D1 | H | H |
| 4115 | 0 | A-26 | H | A-26 | m-D1 | H | H |
| 4116 | 0 | A-27 | H | A-27 | m-D1 | H | H |
| 4117 | 0 | A-28 | H | A-28 | m-D1 | H | H |
| 4118 | 0 | A-29 | H | A-29 | m-D1 | H | H |
| 4119 | 0 | A-30 | H | A-30 | m-D1 | H | H |
| 4120 | 0 | A-31 | H | A-31 | m-D1 | H | H |
| 4121 | 0 | A-32 | H | A-32 | m-D1 | H | H |
| 4122 | 0 | A-33 | H | A-33 | m-D1 | H | H |
| 4123 | 0 | A-34 | H | A-34 | m-D1 | H | H |
| 4124 | 0 | A-35 | H | A-35 | m-D1 | H | H |
| 4125 | 0 | A-36 | H | A-36 | m-D1 | H | H |
| 4126 | 0 | A-37 | H | A-37 | m-D1 | H | H |
| 4127 | 0 | A-38 | H | A-38 | m-D1 | H | H |
| 4128 | 0 | A-39 | H | A-39 | m-D1 | H | H |
| 4129 | 0 | A-40 | H | A-40 | m-D1 | H | H |
| 4130 | 0 | A-41 | H | A-41 | m-D1 | H | H |
| 4131 | 0 | A-42 | H | A-42 | m-D1 | H | H |
| 4132 | 0 | A-43 | H | A-43 | m-D1 | H | H |
| 4133 | 0 | A-44 | H | A-44 | m-D1 | H | H |
| 4134 | 0 | A-45 | H | A-45 | m-D1 | H | H |
| 4135 | 0 | A-46 | H | A-46 | m-D1 | H | H |
| 4136 | 0 | A-47 | H | A-47 | m-D1 | H | H |
| 4137 | 0 | A-1 | H | A-1 | m-D2 | H | H |
| 4138 | 0 | A-2 | H | A-2 | m-D2 | H | H |
| 4139 | 0 | A-3 | H | A-3 | m-D2 | H | H |
| 4140 | 0 | A-4 | H | A-4 | m-D2 | H | H |
| 4141 | 0 | A-5 | H | A-5 | m-D2 | H | H |
| 4142 | 0 | A-6 | H | A-6 | m-D2 | H | H |
| 4143 | 0 | A-7 | H | A-7 | m-D2 | H | H |
| 4144 | 0 | A-8 | H | A-8 | m-D2 | H | H |
| 4145 | 0 | A-9 | H | A-9 | m-D2 | H | H |
| 4146 | 0 | A-10 | H | A-10 | m-D2 | H | H |
| 4147 | 0 | A-11 | H | A-11 | m-D2 | H | H |
| 4148 | 0 | A-12 | H | A-12 | m-D2 | H | H |
| 4149 | 0 | A-13 | H | A-13 | m-D2 | H | H |
| 4150 | 0 | A-14 | H | A-14 | m-D2 | H | H |
| 4151 | 0 | A-15 | H | A-15 | m-D2 | H | H |
| 4152 | 0 | A-16 | H | A-16 | m-D2 | H | H |
| 4153 | 0 | A-17 | H | A-17 | m-D2 | H | H |
| 4154 | 0 | A-18 | H | A-18 | m-D2 | H | H |
| 4155 | 0 | A-19 | H | A-19 | m-D2 | H | H |
| 4156 | 0 | A-20 | H | A-20 | m-D2 | H | H |
| 4157 | 0 | A-21 | H | A-21 | m-D2 | H | H |
| 4158 | 0 | A-22 | H | A-22 | m-D2 | H | H |
| 4159 | 0 | A-23 | H | A-23 | m-D2 | H | H |
| 4160 | 0 | A-24 | H | A-24 | m-D2 | H | H |
| 4161 | 0 | A-25 | H | A-25 | m-D2 | H | H |
| 4162 | 0 | A-26 | H | A-26 | m-D2 | H | H |
| 4163 | 0 | A-27 | H | A-27 | m-D2 | H | H |
| 4164 | 0 | A-28 | H | A-28 | m-D2 | H | H |
| 4165 | 0 | A-29 | H | A-29 | m-D2 | H | H |
| 4166 | 0 | A-30 | H | A-30 | m-D2 | H | H |
| 4167 | 0 | A-31 | H | A-31 | m-D2 | H | H |
| 4168 | 0 | A-32 | H | A-32 | m-D2 | H | H |
| 4169 | 0 | A-33 | H | A-33 | m-D2 | H | H |
| 4170 | 0 | A-34 | H | A-34 | m-D2 | H | H |
| 4171 | 0 | A-35 | H | A-35 | m-D2 | H | H |
| 4172 | 0 | A-36 | H | A-36 | m-D2 | H | H |
| 4173 | 0 | A-37 | H | A-37 | m-D2 | H | H |
| 4174 | 0 | A-38 | H | A-38 | m-D2 | H | H |
| 4175 | 0 | A-39 | H | A-39 | m-D2 | H | H |
| 4176 | 0 | A-40 | H | A-40 | m-D2 | H | H |
| 4177 | 0 | A-41 | H | A-41 | m-D2 | H | H |
| 4178 | 0 | A-42 | H | A-42 | m-D2 | H | H |
| 4179 | 0 | A-43 | H | A-43 | m-D2 | H | H |
| 4180 | 0 | A-44 | H | A-44 | m-D2 | H | H |
| 4181 | 0 | A-45 | H | A-45 | m-D2 | H | H |
| 4182 | 0 | A-46 | H | A-46 | m-D2 | H | H |
| 4183 | 0 | A-47 | H | A-47 | m-D2 | H | H |
| 4184 | 0 | A-1 | H | A-1 | m-D3 | H | H |
| 4185 | 0 | A-2 | H | A-2 | m-D3 | H | H |
| 4186 | 0 | A-3 | H | A-3 | m-D3 | H | H |
| 4187 | 0 | A-4 | H | A-4 | m-D3 | H | H |
| 4188 | 0 | A-5 | H | A-5 | m-D3 | H | H |
| 4189 | 0 | A-6 | H | A-6 | m-D3 | H | H |
| 4190 | 0 | A-7 | H | A-7 | m-D3 | H | H |
| 4191 | 0 | A-8 | H | A-8 | m-D3 | H | H |
| 4192 | 0 | A-9 | H | A-9 | m-D3 | H | H |
| 4193 | 0 | A-10 | H | A-10 | m-D3 | H | H |
| 4194 | 0 | A-11 | H | A-11 | m-D3 | H | H |
| 4195 | 0 | A-12 | H | A-12 | m-D3 | H | H |
| 4196 | 0 | A-13 | H | A-13 | m-D3 | H | H |
| 4197 | 0 | A-14 | H | A-14 | m-D3 | H | H |
| 4198 | 0 | A-15 | H | A-15 | m-D3 | H | H |
| 4199 | 0 | A-16 | H | A-16 | m-D3 | H | H |
| 4200 | 0 | A-17 | H | A-17 | m-D3 | H | H |
| 4201 | 0 | A-18 | H | A-18 | m-D3 | H | H |
| 4202 | 0 | A-19 | H | A-19 | m-D3 | H | H |
| 4203 | 0 | A-20 | H | A-20 | m-D3 | H | H |
| 4204 | 0 | A-21 | H | A-21 | m-D3 | H | H |
| 4205 | 0 | A-22 | H | A-22 | m-D3 | H | H |
| 4206 | 0 | A-23 | H | A-23 | m-D3 | H | H |
| 4207 | 0 | A-24 | H | A-24 | m-D3 | H | H |
| 4208 | 0 | A-25 | H | A-25 | m-D3 | H | H |
| 4209 | 0 | A-26 | H | A-26 | m-D3 | H | H |
| 4210 | 0 | A-27 | H | A-27 | m-D3 | H | H |
| 4211 | 0 | A-28 | H | A-28 | m-D3 | H | H |
| 4212 | 0 | A-29 | H | A-29 | m-D3 | H | H |
| 4213 | 0 | A-30 | H | A-30 | m-D3 | H | H |
| 4214 | 0 | A-31 | H | A-31 | m-D3 | H | H |
| 4215 | 0 | A-32 | H | A-32 | m-D3 | H | H |
| 4216 | 0 | A-33 | H | A-33 | m-D3 | H | H |
| 4217 | 0 | A-34 | H | A-34 | m-D3 | H | H |
| 4218 | 0 | A-35 | H | A-35 | m-D3 | H | H |
| 4219 | 0 | A-36 | H | A-36 | m-D3 | H | H |
| 4220 | 0 | A-37 | H | A-37 | m-D3 | H | H |
| 4221 | 0 | A-38 | H | A-38 | m-D3 | H | H |
| 4222 | 0 | A-39 | H | A-39 | m-D3 | H | H |
| 4223 | 0 | A-40 | H | A-40 | m-D3 | H | H |
| 4224 | 0 | A-41 | H | A-41 | m-D3 | H | H |
| 4225 | 0 | A-42 | H | A-42 | m-D3 | H | H |
| 4226 | 0 | A-43 | H | A-43 | m-D3 | H | H |
| 4227 | 0 | A-44 | H | A-44 | m-D3 | H | H |
| 4228 | 0 | A-45 | H | A-45 | m-D3 | H | H |
| 4229 | 0 | A-46 | H | A-46 | m-D3 | H | H |
| 4230 | 0 | A-47 | H | A-47 | m-D3 | H | H |
| 4231 | 0 | A-1 | H | A-1 | m-D5 | H | H |
| 4232 | 0 | A-2 | H | A-2 | m-D5 | H | H |
| 4233 | 0 | A-3 | H | A-3 | m-D5 | H | H |
| 4234 | 0 | A-4 | H | A-4 | m-D5 | H | H |
| 4235 | 0 | A-5 | H | A-5 | m-D5 | H | H |
| 4236 | 0 | A-6 | H | A-6 | m-D5 | H | H |
| 4237 | 0 | A-7 | H | A-7 | m-D5 | H | H |
| 4238 | 0 | A-8 | H | A-8 | m-D5 | H | H |
| 4239 | 0 | A-9 | H | A-9 | m-D5 | H | H |
| 4240 | 0 | A-10 | H | A-10 | m-D5 | H | H |
| 4241 | 0 | A-11 | H | A-11 | m-D5 | H | H |
| 4242 | 0 | A-12 | H | A-12 | m-D5 | H | H |
| 4243 | 0 | A-13 | H | A-13 | m-D5 | H | H |
| 4244 | 0 | A-14 | H | A-14 | m-D5 | H | H |
| 4245 | 0 | A-15 | H | A-15 | m-D5 | H | H |
| 4246 | 0 | A-16 | H | A-16 | m-D5 | H | H |
| 4247 | 0 | A-17 | H | A-17 | m-D5 | H | H |
| 4248 | 0 | A-18 | H | A-18 | m-D5 | H | H |
| 4249 | 0 | A-19 | H | A-19 | m-D5 | H | H |
| 4250 | 0 | A-20 | H | A-20 | m-D5 | H | H |
| 4251 | 0 | A-21 | H | A-21 | m-D5 | H | H |
| 4252 | 0 | A-22 | H | A-22 | m-D5 | H | H |
| 4253 | 0 | A-23 | H | A-23 | m-D5 | H | H |
| 4254 | 0 | A-24 | H | A-24 | m-D5 | H | H |
| 4255 | 0 | A-25 | H | A-25 | m-D5 | H | H |
| 4256 | 0 | A-26 | H | A-26 | m-D5 | H | H |
| 4257 | 0 | A-27 | H | A-27 | m-D5 | H | H |
| 4258 | 0 | A-28 | H | A-28 | m-D5 | H | H |
| 4259 | 0 | A-29 | H | A-29 | m-D5 | H | H |
| 4260 | 0 | A-30 | H | A-30 | m-D5 | H | H |
| 4261 | 0 | A-31 | H | A-31 | m-D5 | H | H |
| 4262 | 0 | A-32 | H | A-32 | m-D5 | H | H |
| 4263 | 0 | A-33 | H | A-33 | m-D5 | H | H |
| 4264 | 0 | A-34 | H | A-34 | m-D5 | H | H |
| 4265 | 0 | A-35 | H | A-35 | m-D5 | H | H |
| 4266 | 0 | A-36 | H | A-36 | m-D5 | H | H |
| 4267 | 0 | A-37 | H | A-37 | m-D5 | H | H |
| 4268 | 0 | A-38 | H | A-38 | m-D5 | H | H |
| 4269 | 0 | A-39 | H | A-39 | m-D5 | H | H |
| 4270 | 0 | A-40 | H | A-40 | m-D5 | H | H |
| 4271 | 0 | A-41 | H | A-41 | m-D5 | H | H |
| 4272 | 0 | A-42 | H | A-42 | m-D5 | H | H |
| 4273 | 0 | A-43 | H | A-43 | m-D5 | H | H |
| 4274 | 0 | A-44 | H | A-44 | m-D5 | H | H |
| 4275 | 0 | A-45 | H | A-45 | m-D5 | H | H |
| 4276 | 0 | A-46 | H | A-46 | m-D5 | H | H |
| 4277 | 0 | A-47 | H | A-47 | m-D5 | H | H |
| 4278 | 0 | A-1 | H | A-1 | m-D9 | H | H |
| 4279 | 0 | A-2 | H | A-2 | m-D9 | H | H |
| 4280 | 0 | A-3 | H | A-3 | m-D9 | H | H |
| 4281 | 0 | A-4 | H | A-4 | m-D9 | H | H |
| 4282 | 0 | A-5 | H | A-5 | m-D9 | H | H |
| 4283 | 0 | A-6 | H | A-6 | m-D9 | H | H |
| 4284 | 0 | A-7 | H | A-7 | m-D9 | H | H |
| 4285 | 0 | A-8 | H | A-8 | m-D9 | H | H |
| 4286 | 0 | A-9 | H | A-9 | m-D9 | H | H |
| 4287 | 0 | A-10 | H | A-10 | m-D9 | H | H |
| 4288 | 0 | A-11 | H | A-11 | m-D9 | H | H |
| 4289 | 0 | A-12 | H | A-12 | m-D9 | H | H |
| 4290 | 0 | A-13 | H | A-13 | m-D9 | H | H |
| 4291 | 0 | A-14 | H | A-14 | m-D9 | H | H |
| 4292 | 0 | A-15 | H | A-15 | m-D9 | H | H |
| 4293 | 0 | A-16 | H | A-16 | m-D9 | H | H |
| 4294 | 0 | A-17 | H | A-17 | m-D9 | H | H |
| 4295 | 0 | A-18 | H | A-18 | m-D9 | H | H |
| 4296 | 0 | A-19 | H | A-19 | m-D9 | H | H |
| 4297 | 0 | A-20 | H | A-20 | m-D9 | H | H |
| 4298 | 0 | A-21 | H | A-21 | m-D9 | H | H |
| 4299 | 0 | A-22 | H | A-22 | m-D9 | H | H |
| 4300 | 0 | A-23 | H | A-23 | m-D9 | H | H |
| 4301 | 0 | A-24 | H | A-24 | m-D9 | H | H |
| 4302 | 0 | A-25 | H | A-25 | m-D9 | H | H |
| 4303 | 0 | A-26 | H | A-26 | m-D9 | H | H |
| 4304 | 0 | A-27 | H | A-27 | m-D9 | H | H |
| 4305 | 0 | A-28 | H | A-28 | m-D9 | H | H |
| 4306 | 0 | A-29 | H | A-29 | m-D9 | H | H |
| 4307 | 0 | A-30 | H | A-30 | m-D9 | H | H |
| 4308 | 0 | A-31 | H | A-31 | m-D9 | H | H |
| 4309 | 0 | A-32 | H | A-32 | m-D9 | H | H |
| 4310 | 0 | A-33 | H | A-33 | m-D9 | H | H |
| 4311 | 0 | A-34 | H | A-34 | m-D9 | H | H |
| 4312 | 0 | A-35 | H | A-35 | m-D9 | H | H |
| 4313 | 0 | A-36 | H | A-36 | m-D9 | H | H |
| 4314 | 0 | A-37 | H | A-37 | m-D9 | H | H |
| 4315 | 0 | A-38 | H | A-38 | m-D9 | H | H |
| 4316 | 0 | A-39 | H | A-39 | m-D9 | H | H |
| 4317 | 0 | A-40 | H | A-40 | m-D9 | H | H |
| 4318 | 0 | A-41 | H | A-41 | m-D9 | H | H |
| 4319 | 0 | A-42 | H | A-42 | m-D9 | H | H |
| 4320 | 0 | A-43 | H | A-43 | m-D9 | H | H |
| 4321 | 0 | A-44 | H | A-44 | m-D9 | H | H |
| 4322 | 0 | A-45 | H | A-45 | m-D9 | H | H |
| 4323 | 0 | A-46 | H | A-46 | m-D9 | H | H |
| 4324 | 0 | A-47 | H | A-47 | m-D9 | H | H |
| 4325 | 0 | A-1 | H | A-1 | m-D11 | H | H |
| 4326 | 0 | A-2 | H | A-2 | m-D11 | H | H |
| 4327 | 0 | A-3 | H | A-3 | m-D11 | H | H |
| 4328 | 0 | A-4 | H | A-4 | m-D11 | H | H |
| 4329 | 0 | A-5 | H | A-5 | m-D11 | H | H |
| 4330 | 0 | A-6 | H | A-6 | m-D11 | H | H |
| 4331 | 0 | A-7 | H | A-7 | m-D11 | H | H |
| 4332 | 0 | A-8 | H | A-8 | m-D11 | H | H |
| 4333 | 0 | A-9 | H | A-9 | m-D11 | H | H |
| 4334 | 0 | A-10 | H | A-10 | m-D11 | H | H |
| 4335 | 0 | A-11 | H | A-11 | m-D11 | H | H |
| 4336 | 0 | A-12 | H | A-12 | m-D11 | H | H |
| 4337 | 0 | A-13 | H | A-13 | m-D11 | H | H |
| 4338 | 0 | A-14 | H | A-14 | m-D11 | H | H |
| 4339 | 0 | A-15 | H | A-15 | m-D11 | H | H |
| 4340 | 0 | A-16 | H | A-16 | m-D11 | H | H |
| 4341 | 0 | A-17 | H | A-17 | m-D11 | H | H |
| 4342 | 0 | A-18 | H | A-18 | m-D11 | H | H |
| 4343 | 0 | A-19 | H | A-19 | m-D11 | H | H |
| 4344 | 0 | A-20 | H | A-20 | m-D11 | H | H |
| 4345 | 0 | A-21 | H | A-21 | m-D11 | H | H |
| 4346 | 0 | A-22 | H | A-22 | m-D11 | H | H |
| 4347 | 0 | A-23 | H | A-23 | m-D11 | H | H |
| 4348 | 0 | A-24 | H | A-24 | m-D11 | H | H |
| 4349 | 0 | A-25 | H | A-25 | m-D11 | H | H |
| 4350 | 0 | A-26 | H | A-26 | m-D11 | H | H |
| 4351 | 0 | A-27 | H | A-27 | m-D11 | H | H |
| 4352 | 0 | A-28 | H | A-28 | m-D11 | H | H |
| 4353 | 0 | A-29 | H | A-29 | m-D11 | H | H |
| 4354 | 0 | A-30 | H | A-30 | m-D11 | H | H |
| 4355 | 0 | A-31 | H | A-31 | m-D11 | H | H |
| 4356 | 0 | A-32 | H | A-32 | m-D11 | H | H |
| 4357 | 0 | A-33 | H | A-33 | m-D11 | H | H |
| 4358 | 0 | A-34 | H | A-34 | m-D11 | H | H |
| 4359 | 0 | A-35 | H | A-35 | m-D11 | H | H |
| 4360 | 0 | A-36 | H | A-36 | m-D11 | H | H |
| 4361 | 0 | A-37 | H | A-37 | m-D11 | H | H |
| 4362 | 0 | A-38 | H | A-38 | m-D11 | H | H |
| 4363 | 0 | A-39 | H | A-39 | m-D11 | H | H |
| 4364 | 0 | A-40 | H | A-40 | m-D11 | H | H |
| 4365 | 0 | A-41 | H | A-41 | m-D11 | H | H |
| 4366 | 0 | A-42 | H | A-42 | m-D11 | H | H |
| 4367 | 0 | A-43 | H | A-43 | m-D11 | H | H |
| 4368 | 0 | A-44 | H | A-44 | m-D11 | H | H |
| 4369 | 0 | A-45 | H | A-45 | m-D11 | H | H |
| 4370 | 0 | A-46 | H | A-46 | m-D11 | H | H |
| 4371 | 0 | A-47 | H | A-47 | m-D11 | H | H |
| 4372 | 0 | A-1 | H | A-1 | m-D12 | H | H |
| 4373 | 0 | A-2 | H | A-2 | m-D12 | H | H |
| 4374 | 0 | A-3 | H | A-3 | m-D12 | H | H |
| 4375 | 0 | A-4 | H | A-4 | m-D12 | H | H |
| 4376 | 0 | A-5 | H | A-5 | m-D12 | H | H |
| 4377 | 0 | A-6 | H | A-6 | m-D12 | H | H |
| 4378 | 0 | A-7 | H | A-7 | m-D12 | H | H |
| 4379 | 0 | A-8 | H | A-8 | m-D12 | H | H |
| 4380 | 0 | A-9 | H | A-9 | m-D12 | H | H |
| 4381 | 0 | A-10 | H | A-10 | m-D12 | H | H |
| 4382 | 0 | A-11 | H | A-11 | m-D12 | H | H |
| 4383 | 0 | A-12 | H | A-12 | m-D12 | H | H |
| 4384 | 0 | A-13 | H | A-13 | m-D12 | H | H |
| 4385 | 0 | A-14 | H | A-14 | m-D12 | H | H |
| 4386 | 0 | A-15 | H | A-15 | m-D12 | H | H |
| 4387 | 0 | A-16 | H | A-16 | m-D12 | H | H |
| 4388 | 0 | A-17 | H | A-17 | m-D12 | H | H |
| 4389 | 0 | A-18 | H | A-18 | m-D12 | H | H |
| 4390 | 0 | A-19 | H | A-19 | m-D12 | H | H |
| 4391 | 0 | A-20 | H | A-20 | m-D12 | H | H |
| 4392 | 0 | A-21 | H | A-21 | m-D12 | H | H |
| 4393 | 0 | A-22 | H | A-22 | m-D12 | H | H |
| 4394 | 0 | A-23 | H | A-23 | m-D12 | H | H |
| 4395 | 0 | A-24 | H | A-24 | m-D12 | H | H |
| 4396 | 0 | A-25 | H | A-25 | m-D12 | H | H |
| 4397 | 0 | A-26 | H | A-26 | m-D12 | H | H |
| 4398 | 0 | A-27 | H | A-27 | m-D12 | H | H |
| 4399 | 0 | A-28 | H | A-28 | m-D12 | H | H |
| 4400 | 0 | A-29 | H | A-29 | m-D12 | H | H |
| 4401 | 0 | A-30 | H | A-30 | m-D12 | H | H |
| 4402 | 0 | A-31 | H | A-31 | m-D12 | H | H |
| 4403 | 0 | A-32 | H | A-32 | m-D12 | H | H |
| 4404 | 0 | A-33 | H | A-33 | m-D12 | H | H |
| 4405 | 0 | A-34 | H | A-34 | m-D12 | H | H |
| 4406 | 0 | A-35 | H | A-35 | m-D12 | H | H |
| 4407 | 0 | A-36 | H | A-36 | m-D12 | H | H |
| 4408 | 0 | A-37 | H | A-37 | m-D12 | H | H |
| 4409 | 0 | A-38 | H | A-38 | m-D12 | H | H |
| 4410 | 0 | A-39 | H | A-39 | m-D12 | H | H |
| 4411 | 0 | A-40 | H | A-40 | m-D12 | H | H |
| 4412 | 0 | A-41 | H | A-41 | m-D12 | H | H |
| 4413 | 0 | A-42 | H | A-42 | m-D12 | H | H |
| 4414 | 0 | A-43 | H | A-43 | m-D12 | H | H |
| 4415 | 0 | A-44 | H | A-44 | m-D12 | H | H |
| 4416 | 0 | A-45 | H | A-45 | m-D12 | H | H |
| 4417 | 0 | A-46 | H | A-46 | m-D12 | H | H |
| 4418 | 0 | A-47 | H | A-47 | m-D12 | H | H |
| 4419 | 0 | A-1 | H | A-1 | m-D13 | H | H |
| 4420 | 0 | A-2 | H | A-2 | m-D13 | H | H |
| 4421 | 0 | A-3 | H | A-3 | m-D13 | H | H |
| 4422 | 0 | A-4 | H | A-4 | m-D13 | H | H |
| 4423 | 0 | A-5 | H | A-5 | m-D13 | H | H |
| 4424 | 0 | A-6 | H | A-6 | m-D13 | H | H |
| 4425 | 0 | A-7 | H | A-7 | m-D13 | H | H |
| 4426 | 0 | A-8 | H | A-8 | m-D13 | H | H |
| 4427 | 0 | A-9 | H | A-9 | m-D13 | H | H |
| 4428 | 0 | A-10 | H | A-10 | m-D13 | H | H |
| 4429 | 0 | A-11 | H | A-11 | m-D13 | H | H |
| 4430 | 0 | A-12 | H | A-12 | m-D13 | H | H |
| 4431 | 0 | A-13 | H | A-13 | m-D13 | H | H |
| 4432 | 0 | A-14 | H | A-14 | m-D13 | H | H |
| 4433 | 0 | A-15 | H | A-15 | m-D13 | H | H |
| 4434 | 0 | A-16 | H | A-16 | m-D13 | H | H |
| 4435 | 0 | A-17 | H | A-17 | m-D13 | H | H |
| 4436 | 0 | A-18 | H | A-18 | m-D13 | H | H |
| 4437 | 0 | A-19 | H | A-19 | m-D13 | H | H |
| 4438 | 0 | A-20 | H | A-20 | m-D13 | H | H |
| 4439 | 0 | A-21 | H | A-21 | m-D13 | H | H |
| 4440 | 0 | A-22 | H | A-22 | m-D13 | H | H |
| 4441 | 0 | A-23 | H | A-23 | m-D13 | H | H |
| 4442 | 0 | A-24 | H | A-24 | m-D13 | H | H |
| 4443 | 0 | A-25 | H | A-25 | m-D13 | H | H |
| 4444 | 0 | A-26 | H | A-26 | m-D13 | H | H |
| 4445 | 0 | A-27 | H | A-27 | m-D13 | H | H |
| 4446 | 0 | A-28 | H | A-28 | m-D13 | H | H |
| 4447 | 0 | A-29 | H | A-29 | m-D13 | H | H |
| 4448 | 0 | A-30 | H | A-30 | m-D13 | H | H |
| 4449 | 0 | A-31 | H | A-31 | m-D13 | H | H |
| 4450 | 0 | A-32 | H | A-32 | m-D13 | H | H |
| 4451 | 0 | A-33 | H | A-33 | m-D13 | H | H |
| 4452 | 0 | A-34 | H | A-34 | m-D13 | H | H |
| 4453 | 0 | A-35 | H | A-35 | m-D13 | H | H |
| 4454 | 0 | A-36 | H | A-36 | m-D13 | H | H |
| 4455 | 0 | A-37 | H | A-37 | m-D13 | H | H |
| 4456 | 0 | A-38 | H | A-38 | m-D13 | H | H |
| 4457 | 0 | A-39 | H | A-39 | m-D13 | H | H |
| 4458 | 0 | A-40 | H | A-40 | m-D13 | H | H |
| 4459 | 0 | A-41 | H | A-41 | m-D13 | H | H |
| 4460 | 0 | A-42 | H | A-42 | m-D13 | H | H |
| 4461 | 0 | A-43 | H | A-43 | m-D13 | H | H |
| 4462 | 0 | A-44 | H | A-44 | m-D13 | H | H |
| 4463 | 0 | A-45 | H | A-45 | m-D13 | H | H |
| 4464 | 0 | A-46 | H | A-46 | m-D13 | H | H |
| 4465 | 0 | A-47 | H | A-47 | m-D13 | H | H |
| 4466 | 0 | A-1 | H | A-1 | m-D14 | H | H |
| 4467 | 0 | A-2 | H | A-2 | m-D14 | H | H |
| 4468 | 0 | A-3 | H | A-3 | m-D14 | H | H |
| 4469 | 0 | A-4 | H | A-4 | m-D14 | H | H |
| 4470 | 0 | A-5 | H | A-5 | m-D14 | H | H |
| 4471 | 0 | A-6 | H | A-6 | m-D14 | H | H |
| 4472 | 0 | A-7 | H | A-7 | m-D14 | H | H |
| 4473 | 0 | A-8 | H | A-8 | m-D14 | H | H |
| 4474 | 0 | A-9 | H | A-9 | m-D14 | H | H |
| 4475 | 0 | A-10 | H | A-10 | m-D14 | H | H |
| 4476 | 0 | A-11 | H | A-11 | m-D14 | H | H |
| 4477 | 0 | A-12 | H | A-12 | m-D14 | H | H |
| 4478 | 0 | A-13 | H | A-13 | m-D14 | H | H |
| 4479 | 0 | A-14 | H | A-14 | m-D14 | H | H |
| 4480 | 0 | A-15 | H | A-15 | m-D14 | H | H |
| 4481 | 0 | A-16 | H | A-16 | m-D14 | H | H |
| 4482 | 0 | A-17 | H | A-17 | m-D14 | H | H |
| 4483 | 0 | A-18 | H | A-18 | m-D14 | H | H |
| 4484 | 0 | A-19 | H | A-19 | m-D14 | H | H |
| 4485 | 0 | A-20 | H | A-20 | m-D14 | H | H |
| 4486 | 0 | A-21 | H | A-21 | m-D14 | H | H |
| 4487 | 0 | A-22 | H | A-22 | m-D14 | H | H |
| 4488 | 0 | A-23 | H | A-23 | m-D14 | H | H |
| 4489 | 0 | A-24 | H | A-24 | m-D14 | H | H |
| 4490 | 0 | A-25 | H | A-25 | m-D14 | H | H |
| 4491 | 0 | A-26 | H | A-26 | m-D14 | H | H |
| 4492 | 0 | A-27 | H | A-27 | m-D14 | H | H |
| 4493 | 0 | A-28 | H | A-28 | m-D14 | H | H |
| 4494 | 0 | A-29 | H | A-29 | m-D14 | H | H |
| 4495 | 0 | A-30 | H | A-30 | m-D14 | H | H |
| 4496 | 0 | A-31 | H | A-31 | m-D14 | H | H |
| 4497 | 0 | A-32 | H | A-32 | m-D14 | H | H |
| 4498 | 0 | A-33 | H | A-33 | m-D14 | H | H |
| 4499 | 0 | A-34 | H | A-34 | m-D14 | H | H |
| 4500 | 0 | A-35 | H | A-35 | m-D14 | H | H |
| 4501 | 0 | A-36 | H | A-36 | m-D14 | H | H |
| 4502 | 0 | A-37 | H | A-37 | m-D14 | H | H |
| 4503 | 0 | A-38 | H | A-38 | m-D14 | H | H |
| 4504 | 0 | A-39 | H | A-39 | m-D14 | H | H |
| 4505 | 0 | A-40 | H | A-40 | m-D14 | H | H |
| 4506 | 0 | A-41 | H | A-41 | m-D14 | H | H |
| 4507 | 0 | A-42 | H | A-42 | m-D14 | H | H |
| 4508 | 0 | A-43 | H | A-43 | m-D14 | H | H |
| 4509 | 0 | A-44 | H | A-44 | m-D14 | H | H |
| 4510 | 0 | A-45 | H | A-45 | m-D14 | H | H |
| 4511 | 0 | A-46 | H | A-46 | m-D14 | H | H |
| 4512 | 0 | A-47 | H | A-47 | m-D14 | H | H |
| 4513 | 0 | A-1 | H | m-D1 | A-1 | H | H |
| 4514 | 0 | A-2 | H | m-D1 | A-2 | H | H |
| 4515 | 0 | A-3 | H | m-D1 | A-3 | H | H |
| 4516 | 0 | A-4 | H | m-D1 | A-4 | H | H |
| 4517 | 0 | A-5 | H | m-D1 | A-5 | H | H |
| 4518 | 0 | A-6 | H | m-D1 | A-6 | H | H |
| 4519 | 0 | A-7 | H | m-D1 | A-7 | H | H |
| 4520 | 0 | A-8 | H | m-D1 | A-8 | H | H |
| 4521 | 0 | A-9 | H | m-D1 | A-9 | H | H |
| 4522 | 0 | A-10 | H | m-D1 | A-10 | H | H |
| 4523 | 0 | A-11 | H | m-D1 | A-11 | H | H |
| 4524 | 0 | A-12 | H | m-D1 | A-12 | H | H |
| 4525 | 0 | A-13 | H | m-D1 | A-13 | H | H |
| 4526 | 0 | A-14 | H | m-D1 | A-14 | H | H |
| 4527 | 0 | A-15 | H | m-D1 | A-15 | H | H |
| 4528 | 0 | A-16 | H | m-D1 | A-16 | H | H |
| 4529 | 0 | A-17 | H | m-D1 | A-17 | H | H |
| 4530 | 0 | A-18 | H | m-D1 | A-18 | H | H |
| 4531 | 0 | A-19 | H | m-D1 | A-19 | H | H |
| 4532 | 0 | A-20 | H | m-D1 | A-20 | H | H |
| 4533 | 0 | A-21 | H | m-D1 | A-21 | H | H |
| 4534 | 0 | A-22 | H | m-D1 | A-22 | H | H |
| 4535 | 0 | A-23 | H | m-D1 | A-23 | H | H |
| 4536 | 0 | A-24 | H | m-D1 | A-24 | H | H |
| 4537 | 0 | A-25 | H | m-D1 | A-25 | H | H |
| 4538 | 0 | A-26 | H | m-D1 | A-26 | H | H |
| 4539 | 0 | A-27 | H | m-D1 | A-27 | H | H |
| 4540 | 0 | A-28 | H | m-D1 | A-28 | H | H |
| 4541 | 0 | A-29 | H | m-D1 | A-29 | H | H |
| 4542 | 0 | A-30 | H | m-D1 | A-30 | H | H |
| 4543 | 0 | A-31 | H | m-D1 | A-31 | H | H |
| 4544 | 0 | A-32 | H | m-D1 | A-32 | H | H |
| 4545 | 0 | A-33 | H | m-D1 | A-33 | H | H |
| 4546 | 0 | A-34 | H | m-D1 | A-34 | H | H |
| 4547 | 0 | A-35 | H | m-D1 | A-35 | H | H |
| 4548 | 0 | A-36 | H | m-D1 | A-36 | H | H |
| 4549 | 0 | A-37 | H | m-D1 | A-37 | H | H |
| 4550 | 0 | A-38 | H | m-D1 | A-38 | H | H |
| 4551 | 0 | A-39 | H | m-D1 | A-39 | H | H |
| 4552 | 0 | A-40 | H | m-D1 | A-40 | H | H |
| 4553 | 0 | A-41 | H | m-D1 | A-41 | H | H |
| 4554 | 0 | A-42 | H | m-D1 | A-42 | H | H |
| 4555 | 0 | A-43 | H | m-D1 | A-43 | H | H |
| 4556 | 0 | A-44 | H | m-D1 | A-44 | H | H |
| 4557 | 0 | A-45 | H | m-D1 | A-45 | H | H |
| 4558 | 0 | A-46 | H | m-D1 | A-46 | H | H |
| 4559 | 0 | A-47 | H | m-D1 | A-47 | H | H |
| 4560 | 0 | A-1 | H | m-D2 | A-1 | H | H |
| 4561 | 0 | A-2 | H | m-D2 | A-2 | H | H |
| 4562 | 0 | A-3 | H | m-D2 | A-3 | H | H |
| 4563 | 0 | A-4 | H | m-D2 | A-4 | H | H |
| 4564 | 0 | A-5 | H | m-D2 | A-5 | H | H |
| 4565 | 0 | A-6 | H | m-D2 | A-6 | H | H |
| 4566 | 0 | A-7 | H | m-D2 | A-7 | H | H |
| 4567 | 0 | A-8 | H | m-D2 | A-8 | H | H |
| 4568 | 0 | A-9 | H | m-D2 | A-9 | H | H |
| 4569 | 0 | A-10 | H | m-D2 | A-10 | H | H |
| 4570 | 0 | A-11 | H | m-D2 | A-11 | H | H |
| 4571 | 0 | A-12 | H | m-D2 | A-12 | H | H |
| 4572 | 0 | A-13 | H | m-D2 | A-13 | H | H |
| 4573 | 0 | A-14 | H | m-D2 | A-14 | H | H |
| 4574 | 0 | A-15 | H | m-D2 | A-15 | H | H |
| 4575 | 0 | A-16 | H | m-D2 | A-16 | H | H |
| 4576 | 0 | A-17 | H | m-D2 | A-17 | H | H |
| 4577 | 0 | A-18 | H | m-D2 | A-18 | H | H |
| 4578 | 0 | A-19 | H | m-D2 | A-19 | H | H |
| 4579 | 0 | A-20 | H | m-D2 | A-20 | H | H |
| 4580 | 0 | A-21 | H | m-D2 | A-21 | H | H |
| 4581 | 0 | A-22 | H | m-D2 | A-22 | H | H |
| 4582 | 0 | A-23 | H | m-D2 | A-23 | H | H |
| 4583 | 0 | A-24 | H | m-D2 | A-24 | H | H |
| 4584 | 0 | A-25 | H | m-D2 | A-25 | H | H |
| 4585 | 0 | A-26 | H | m-D2 | A-26 | H | H |
| 4586 | 0 | A-27 | H | m-D2 | A-27 | H | H |
| 4587 | 0 | A-28 | H | m-D2 | A-28 | H | H |
| 4588 | 0 | A-29 | H | m-D2 | A-29 | H | H |
| 4589 | 0 | A-30 | H | m-D2 | A-30 | H | H |
| 4590 | 0 | A-31 | H | m-D2 | A-31 | H | H |
| 4591 | 0 | A-32 | H | m-D2 | A-32 | H | H |
| 4592 | 0 | A-33 | H | m-D2 | A-33 | H | H |
| 4593 | 0 | A-34 | H | m-D2 | A-34 | H | H |
| 4594 | 0 | A-35 | H | m-D2 | A-35 | H | H |
| 4595 | 0 | A-36 | H | m-D2 | A-36 | H | H |
| 4596 | 0 | A-37 | H | m-D2 | A-37 | H | H |
| 4597 | 0 | A-38 | H | m-D2 | A-38 | H | H |
| 4598 | 0 | A-39 | H | m-D2 | A-39 | H | H |
| 4599 | 0 | A-40 | H | m-D2 | A-40 | H | H |
| 4600 | 0 | A-41 | H | m-D2 | A-41 | H | H |
| 4601 | 0 | A-42 | H | m-D2 | A-42 | H | H |
| 4602 | 0 | A-43 | H | m-D2 | A-43 | H | H |
| 4603 | 0 | A-44 | H | m-D2 | A-44 | H | H |
| 4604 | 0 | A-45 | H | m-D2 | A-45 | H | H |
| 4605 | 0 | A-46 | H | m-D2 | A-46 | H | H |
| 4606 | 0 | A-47 | H | m-D2 | A-47 | H | H |
| 4607 | 0 | A-1 | H | m-D3 | A-1 | H | H |
| 4608 | 0 | A-2 | H | m-D3 | A-2 | H | H |
| 4609 | 0 | A-3 | H | m-D3 | A-3 | H | H |
| 4610 | 0 | A-4 | H | m-D3 | A-4 | H | H |
| 4611 | 0 | A-5 | H | m-D3 | A-5 | H | H |
| 4612 | 0 | A-6 | H | m-D3 | A-6 | H | H |
| 4613 | 0 | A-7 | H | m-D3 | A-7 | H | H |
| 4614 | 0 | A-8 | H | m-D3 | A-8 | H | H |
| 4615 | 0 | A-9 | H | m-D3 | A-9 | H | H |
| 4616 | 0 | A-10 | H | m-D3 | A-10 | H | H |
| 4617 | 0 | A-11 | H | m-D3 | A-11 | H | H |
| 4618 | 0 | A-12 | H | m-D3 | A-12 | H | H |
| 4619 | 0 | A-13 | H | m-D3 | A-13 | H | H |
| 4620 | 0 | A-14 | H | m-D3 | A-14 | H | H |
| 4621 | 0 | A-15 | H | m-D3 | A-15 | H | H |
| 4622 | 0 | A-16 | H | m-D3 | A-16 | H | H |
| 4623 | 0 | A-17 | H | m-D3 | A-17 | H | H |
| 4624 | 0 | A-18 | H | m-D3 | A-18 | H | H |
| 4625 | 0 | A-19 | H | m-D3 | A-19 | H | H |
| 4626 | 0 | A-20 | H | m-D3 | A-20 | H | H |
| 4627 | 0 | A-21 | H | m-D3 | A-21 | H | H |
| 4628 | 0 | A-22 | H | m-D3 | A-22 | H | H |
| 4629 | 0 | A-23 | H | m-D3 | A-23 | H | H |
| 4630 | 0 | A-24 | H | m-D3 | A-24 | H | H |
| 4631 | 0 | A-25 | H | m-D3 | A-25 | H | H |
| 4632 | 0 | A-26 | H | m-D3 | A-26 | H | H |
| 4633 | 0 | A-27 | H | m-D3 | A-27 | H | H |
| 4634 | 0 | A-28 | H | m-D3 | A-28 | H | H |
| 4635 | 0 | A-29 | H | m-D3 | A-29 | H | H |
| 4636 | 0 | A-30 | H | m-D3 | A-30 | H | H |
| 4637 | 0 | A-31 | H | m-D3 | A-31 | H | H |
| 4638 | 0 | A-32 | H | m-D3 | A-32 | H | H |
| 4639 | 0 | A-33 | H | m-D3 | A-33 | H | H |
| 4640 | 0 | A-34 | H | m-D3 | A-34 | H | H |
| 4641 | 0 | A-35 | H | m-D3 | A-35 | H | H |
| 4642 | 0 | A-36 | H | m-D3 | A-36 | H | H |
| 4643 | 0 | A-37 | H | m-D3 | A-37 | H | H |
| 4644 | 0 | A-38 | H | m-D3 | A-38 | H | H |
| 4645 | 0 | A-39 | H | m-D3 | A-39 | H | H |
| 4646 | 0 | A-40 | H | m-D3 | A-40 | H | H |
| 4647 | 0 | A-41 | H | m-D3 | A-41 | H | H |
| 4648 | 0 | A-42 | H | m-D3 | A-42 | H | H |
| 4649 | 0 | A-43 | H | m-D3 | A-43 | H | H |
| 4650 | 0 | A-44 | H | m-D3 | A-44 | H | H |
| 4651 | 0 | A-45 | H | m-D3 | A-45 | H | H |
| 4652 | 0 | A-46 | H | m-D3 | A-46 | H | H |
| 4653 | 0 | A-47 | H | m-D3 | A-47 | H | H |
| 4654 | 0 | A-1 | H | m-D5 | A-1 | H | H |
| 4655 | 0 | A-2 | H | m-D5 | A-2 | H | H |
| 4656 | 0 | A-3 | H | m-D5 | A-3 | H | H |
| 4657 | 0 | A-4 | H | m-D5 | A-4 | H | H |
| 4658 | 0 | A-5 | H | m-D5 | A-5 | H | H |
| 4659 | 0 | A-6 | H | m-D5 | A-6 | H | H |
| 4660 | 0 | A-7 | H | m-D5 | A-7 | H | H |
| 4661 | 0 | A-8 | H | m-D5 | A-8 | H | H |
| 4662 | 0 | A-9 | H | m-D5 | A-9 | H | H |
| 4663 | 0 | A-10 | H | m-D5 | A-10 | H | H |
| 4664 | 0 | A-11 | H | m-D5 | A-11 | H | H |
| 4665 | 0 | A-12 | H | m-D5 | A-12 | H | H |
| 4666 | 0 | A-13 | H | m-D5 | A-13 | H | H |
| 4667 | 0 | A-14 | H | m-D5 | A-14 | H | H |
| 4668 | 0 | A-15 | H | m-D5 | A-15 | H | H |
| 4669 | 0 | A-16 | H | m-D5 | A-16 | H | H |
| 4670 | 0 | A-17 | H | m-D5 | A-17 | H | H |
| 4671 | 0 | A-18 | H | m-D5 | A-18 | H | H |
| 4672 | 0 | A-19 | H | m-D5 | A-19 | H | H |
| 4673 | 0 | A-20 | H | m-D5 | A-20 | H | H |
| 4674 | 0 | A-21 | H | m-D5 | A-21 | H | H |
| 4675 | 0 | A-22 | H | m-D5 | A-22 | H | H |
| 4676 | 0 | A-23 | H | m-D5 | A-23 | H | H |
| 4677 | 0 | A-24 | H | m-D5 | A-24 | H | H |
| 4678 | 0 | A-25 | H | m-D5 | A-25 | H | H |
| 4679 | 0 | A-26 | H | m-D5 | A-26 | H | H |
| 4680 | 0 | A-27 | H | m-D5 | A-27 | H | H |
| 4681 | 0 | A-28 | H | m-D5 | A-28 | H | H |
| 4682 | 0 | A-29 | H | m-D5 | A-29 | H | H |
| 4683 | 0 | A-30 | H | m-D5 | A-30 | H | H |
| 4684 | 0 | A-31 | H | m-D5 | A-31 | H | H |
| 4685 | 0 | A-32 | H | m-D5 | A-32 | H | H |
| 4686 | 0 | A-33 | H | m-D5 | A-33 | H | H |
| 4687 | 0 | A-34 | H | m-D5 | A-34 | H | H |
| 4688 | 0 | A-35 | H | m-D5 | A-35 | H | H |
| 4689 | 0 | A-36 | H | m-D5 | A-36 | H | H |
| 4690 | 0 | A-37 | H | m-D5 | A-37 | H | H |
| 4691 | 0 | A-38 | H | m-D5 | A-38 | H | H |
| 4692 | 0 | A-39 | H | m-D5 | A-39 | H | H |
| 4693 | 0 | A-40 | H | m-D5 | A-40 | H | H |
| 4694 | 0 | A-41 | H | m-D5 | A-41 | H | H |
| 4695 | 0 | A-42 | H | m-D5 | A-42 | H | H |
| 4696 | 0 | A-43 | H | m-D5 | A-43 | H | H |
| 4697 | 0 | A-44 | H | m-D5 | A-44 | H | H |
| 4698 | 0 | A-45 | H | m-D5 | A-45 | H | H |
| 4699 | 0 | A-46 | H | m-D5 | A-46 | H | H |
| 4700 | 0 | A-47 | H | m-D5 | A-47 | H | H |
| 4701 | 0 | A-1 | H | m-D9 | A-1 | H | H |
| 4702 | 0 | A-2 | H | m-D9 | A-2 | H | H |
| 4703 | 0 | A-3 | H | m-D9 | A-3 | H | H |
| 4704 | 0 | A-4 | H | m-D9 | A-4 | H | H |
| 4705 | 0 | A-5 | H | m-D9 | A-5 | H | H |
| 4706 | 0 | A-6 | H | m-D9 | A-6 | H | H |
| 4707 | 0 | A-7 | H | m-D9 | A-7 | H | H |
| 4708 | 0 | A-8 | H | m-D9 | A-8 | H | H |
| 4709 | 0 | A-9 | H | m-D9 | A-9 | H | H |
| 4710 | 0 | A-10 | H | m-D9 | A-10 | H | H |
| 4711 | 0 | A-11 | H | m-D9 | A-11 | H | H |
| 4712 | 0 | A-12 | H | m-D9 | A-12 | H | H |
| 4713 | 0 | A-13 | H | m-D9 | A-13 | H | H |
| 4714 | 0 | A-14 | H | m-D9 | A-14 | H | H |
| 4715 | 0 | A-15 | H | m-D9 | A-15 | H | H |
| 4716 | 0 | A-16 | H | m-D9 | A-16 | H | H |
| 4717 | 0 | A-17 | H | m-D9 | A-17 | H | H |
| 4718 | 0 | A-18 | H | m-D9 | A-18 | H | H |
| 4719 | 0 | A-19 | H | m-D9 | A-19 | H | H |
| 4720 | 0 | A-20 | H | m-D9 | A-20 | H | H |
| 4721 | 0 | A-21 | H | m-D9 | A-21 | H | H |
| 4722 | 0 | A-22 | H | m-D9 | A-22 | H | H |
| 4723 | 0 | A-23 | H | m-D9 | A-23 | H | H |
| 4724 | 0 | A-24 | H | m-D9 | A-24 | H | H |
| 4725 | 0 | A-25 | H | m-D9 | A-25 | H | H |
| 4726 | 0 | A-26 | H | m-D9 | A-26 | H | H |
| 4727 | 0 | A-27 | H | m-D9 | A-27 | H | H |
| 4728 | 0 | A-28 | H | m-D9 | A-28 | H | H |
| 4729 | 0 | A-29 | H | m-D9 | A-29 | H | H |
| 4730 | 0 | A-30 | H | m-D9 | A-30 | H | H |
| 4731 | 0 | A-31 | H | m-D9 | A-31 | H | H |
| 4732 | 0 | A-32 | H | m-D9 | A-32 | H | H |
| 4733 | 0 | A-33 | H | m-D9 | A-33 | H | H |
| 4734 | 0 | A-34 | H | m-D9 | A-34 | H | H |
| 4735 | 0 | A-35 | H | m-D9 | A-35 | H | H |
| 4736 | 0 | A-36 | H | m-D9 | A-36 | H | H |
| 4737 | 0 | A-37 | H | m-D9 | A-37 | H | H |
| 4738 | 0 | A-38 | H | m-D9 | A-38 | H | H |
| 4739 | 0 | A-39 | H | m-D9 | A-39 | H | H |
| 4740 | 0 | A-40 | H | m-D9 | A-40 | H | H |
| 4741 | 0 | A-41 | H | m-D9 | A-41 | H | H |
| 4742 | 0 | A-42 | H | m-D9 | A-42 | H | H |
| 4743 | 0 | A-43 | H | m-D9 | A-43 | H | H |
| 4744 | 0 | A-44 | H | m-D9 | A-44 | H | H |
| 4745 | 0 | A-45 | H | m-D9 | A-45 | H | H |
| 4746 | 0 | A-46 | H | m-D9 | A-46 | H | H |
| 4747 | 0 | A-47 | H | m-D9 | A-47 | H | H |
| 4748 | 0 | A-1 | H | m-D11 | A-1 | H | H |
| 4749 | 0 | A-2 | H | m-D11 | A-2 | H | H |
| 4750 | 0 | A-3 | H | m-D11 | A-3 | H | H |
| 4751 | 0 | A-4 | H | m-D11 | A-4 | H | H |
| 4752 | 0 | A-5 | H | m-D11 | A-5 | H | H |
| 4753 | 0 | A-6 | H | m-D11 | A-6 | H | H |
| 4754 | 0 | A-7 | H | m-D11 | A-7 | H | H |
| 4755 | 0 | A-8 | H | m-D11 | A-8 | H | H |
| 4756 | 0 | A-9 | H | m-D11 | A-9 | H | H |
| 4757 | 0 | A-10 | H | m-D11 | A-10 | H | H |
| 4758 | 0 | A-11 | H | m-D11 | A-11 | H | H |
| 4759 | 0 | A-12 | H | m-D11 | A-12 | H | H |
| 4760 | 0 | A-13 | H | m-D11 | A-13 | H | H |
| 4761 | 0 | A-14 | H | m-D11 | A-14 | H | H |
| 4762 | 0 | A-15 | H | m-D11 | A-15 | H | H |
| 4763 | 0 | A-16 | H | m-D11 | A-16 | H | H |
| 4764 | 0 | A-17 | H | m-D11 | A-17 | H | H |
| 4765 | 0 | A-18 | H | m-D11 | A-18 | H | H |
| 4766 | 0 | A-19 | H | m-D11 | A-19 | H | H |
| 4767 | 0 | A-20 | H | m-D11 | A-20 | H | H |
| 4768 | 0 | A-21 | H | m-D11 | A-21 | H | H |
| 4769 | 0 | A-22 | H | m-D11 | A-22 | H | H |
| 4770 | 0 | A-23 | H | m-D11 | A-23 | H | H |
| 4771 | 0 | A-24 | H | m-D11 | A-24 | H | H |
| 4772 | 0 | A-25 | H | m-D11 | A-25 | H | H |
| 4773 | 0 | A-26 | H | m-D11 | A-26 | H | H |
| 4774 | 0 | A-27 | H | m-D11 | A-27 | H | H |
| 4775 | 0 | A-28 | H | m-D11 | A-28 | H | H |
| 4776 | 0 | A-29 | H | m-D11 | A-29 | H | H |
| 4777 | 0 | A-30 | H | m-D11 | A-30 | H | H |
| 4778 | 0 | A-31 | H | m-D11 | A-31 | H | H |
| 4779 | 0 | A-32 | H | m-D11 | A-32 | H | H |
| 4780 | 0 | A-33 | H | m-D11 | A-33 | H | H |
| 4781 | 0 | A-34 | H | m-D11 | A-34 | H | H |
| 4782 | 0 | A-35 | H | m-D11 | A-35 | H | H |
| 4783 | 0 | A-36 | H | m-D11 | A-36 | H | H |
| 4784 | 0 | A-37 | H | m-D11 | A-37 | H | H |
| 4785 | 0 | A-38 | H | m-D11 | A-38 | H | H |
| 4786 | 0 | A-39 | H | m-D11 | A-39 | H | H |
| 4787 | 0 | A-40 | H | m-D11 | A-40 | H | H |
| 4788 | 0 | A-41 | H | m-D11 | A-41 | H | H |
| 4789 | 0 | A-42 | H | m-D11 | A-42 | H | H |
| 4790 | 0 | A-43 | H | m-D11 | A-43 | H | H |
| 4791 | 0 | A-44 | H | m-D11 | A-44 | H | H |
| 4792 | 0 | A-45 | H | m-D11 | A-45 | H | H |
| 4793 | 0 | A-46 | H | m-D11 | A-46 | H | H |
| 4794 | 0 | A-47 | H | m-D11 | A-47 | H | H |
| 4795 | 0 | A-1 | H | m-D12 | A-1 | H | H |
| 4796 | 0 | A-2 | H | m-D12 | A-2 | H | H |
| 4797 | 0 | A-3 | H | m-D12 | A-3 | H | H |
| 4798 | 0 | A-4 | H | m-D12 | A-4 | H | H |
| 4799 | 0 | A-5 | H | m-D12 | A-5 | H | H |
| 4800 | 0 | A-6 | H | m-D12 | A-6 | H | H |
| 4801 | 0 | A-7 | H | m-D12 | A-7 | H | H |
| 4802 | 0 | A-8 | H | m-D12 | A-8 | H | H |
| 4803 | 0 | A-9 | H | m-D12 | A-9 | H | H |
| 4804 | 0 | A-10 | H | m-D12 | A-10 | H | H |
| 4805 | 0 | A-11 | H | m-D12 | A-11 | H | H |
| 4806 | 0 | A-12 | H | m-D12 | A-12 | H | H |
| 4807 | 0 | A-13 | H | m-D12 | A-13 | H | H |
| 4808 | 0 | A-14 | H | m-D12 | A-14 | H | H |
| 4809 | 0 | A-15 | H | m-D12 | A-15 | H | H |
| 4810 | 0 | A-16 | H | m-D12 | A-16 | H | H |
| 4811 | 0 | A-17 | H | m-D12 | A-17 | H | H |
| 4812 | 0 | A-18 | H | m-D12 | A-18 | H | H |
| 4813 | 0 | A-19 | H | m-D12 | A-19 | H | H |
| 4814 | 0 | A-20 | H | m-D12 | A-20 | H | H |
| 4815 | 0 | A-21 | H | m-D12 | A-21 | H | H |
| 4816 | 0 | A-22 | H | m-D12 | A-22 | H | H |
| 4817 | 0 | A-23 | H | m-D12 | A-23 | H | H |
| 4818 | 0 | A-24 | H | m-D12 | A-24 | H | H |
| 4819 | 0 | A-25 | H | m-D12 | A-25 | H | H |
| 4820 | 0 | A-26 | H | m-D12 | A-26 | H | H |
| 4821 | 0 | A-27 | H | m-D12 | A-27 | H | H |
| 4822 | 0 | A-28 | H | m-D12 | A-28 | H | H |
| 4823 | 0 | A-29 | H | m-D12 | A-29 | H | H |
| 4824 | 0 | A-30 | H | m-D12 | A-30 | H | H |
| 4825 | 0 | A-31 | H | m-D12 | A-31 | H | H |
| 4826 | 0 | A-32 | H | m-D12 | A-32 | H | H |
| 4827 | 0 | A-33 | H | m-D12 | A-33 | H | H |
| 4828 | 0 | A-34 | H | m-D12 | A-34 | H | H |
| 4829 | 0 | A-35 | H | m-D12 | A-35 | H | H |
| 4830 | 0 | A-36 | H | m-D12 | A-36 | H | H |
| 4831 | 0 | A-37 | H | m-D12 | A-37 | H | H |
| 4832 | 0 | A-38 | H | m-D12 | A-38 | H | H |
| 4833 | 0 | A-39 | H | m-D12 | A-39 | H | H |
| 4834 | 0 | A-40 | H | m-D12 | A-40 | H | H |
| 4835 | 0 | A-41 | H | m-D12 | A-41 | H | H |
| 4836 | 0 | A-42 | H | m-D12 | A-42 | H | H |
| 4837 | 0 | A-43 | H | m-D12 | A-43 | H | H |
| 4838 | 0 | A-44 | H | m-D12 | A-44 | H | H |
| 4839 | 0 | A-45 | H | m-D12 | A-45 | H | H |
| 4840 | 0 | A-46 | H | m-D12 | A-46 | H | H |
| 4841 | 0 | A-47 | H | m-D12 | A-47 | H | H |
| 4842 | 0 | A-1 | H | m-D13 | A-1 | H | H |
| 4843 | 0 | A-2 | H | m-D13 | A-2 | H | H |
| 4844 | 0 | A-3 | H | m-D13 | A-3 | H | H |
| 4845 | 0 | A-4 | H | m-D13 | A-4 | H | H |
| 4846 | 0 | A-5 | H | m-D13 | A-5 | H | H |
| 4847 | 0 | A-6 | H | m-D13 | A-6 | H | H |
| 4848 | 0 | A-7 | H | m-D13 | A-7 | H | H |
| 4849 | 0 | A-8 | H | m-D13 | A-8 | H | H |
| 4850 | 0 | A-9 | H | m-D13 | A-9 | H | H |
| 4851 | 0 | A-10 | H | m-D13 | A-10 | H | H |
| 4852 | 0 | A-11 | H | m-D13 | A-11 | H | H |
| 4853 | 0 | A-12 | H | m-D13 | A-12 | H | H |
| 4854 | 0 | A-13 | H | m-D13 | A-13 | H | H |
| 4855 | 0 | A-14 | H | m-D13 | A-14 | H | H |
| 4856 | 0 | A-15 | H | m-D13 | A-15 | H | H |
| 4857 | 0 | A-16 | H | m-D13 | A-16 | H | H |
| 4858 | 0 | A-17 | H | m-D13 | A-17 | H | H |
| 4859 | 0 | A-18 | H | m-D13 | A-18 | H | H |
| 4860 | 0 | A-19 | H | m-D13 | A-19 | H | H |
| 4861 | 0 | A-20 | H | m-D13 | A-20 | H | H |
| 4862 | 0 | A-21 | H | m-D13 | A-21 | H | H |
| 4863 | 0 | A-22 | H | m-D13 | A-22 | H | H |
| 4864 | 0 | A-23 | H | m-D13 | A-23 | H | H |
| 4865 | 0 | A-24 | H | m-D13 | A-24 | H | H |
| 4866 | 0 | A-25 | H | m-D13 | A-25 | H | H |
| 4867 | 0 | A-26 | H | m-D13 | A-26 | H | H |
| 4868 | 0 | A-27 | H | m-D13 | A-27 | H | H |
| 4869 | 0 | A-28 | H | m-D13 | A-28 | H | H |
| 4870 | 0 | A-29 | H | m-D13 | A-29 | H | H |
| 4871 | 0 | A-30 | H | m-D13 | A-30 | H | H |
| 4872 | 0 | A-31 | H | m-D13 | A-31 | H | H |
| 4873 | 0 | A-32 | H | m-D13 | A-32 | H | H |
| 4874 | 0 | A-33 | H | m-D13 | A-33 | H | H |
| 4875 | 0 | A-34 | H | m-D13 | A-34 | H | H |
| 4876 | 0 | A-35 | H | m-D13 | A-35 | H | H |
| 4877 | 0 | A-36 | H | m-D13 | A-36 | H | H |
| 4878 | 0 | A-37 | H | m-D13 | A-37 | H | H |
| 4879 | 0 | A-38 | H | m-D13 | A-38 | H | H |
| 4880 | 0 | A-39 | H | m-D13 | A-39 | H | H |
| 4881 | 0 | A-40 | H | m-D13 | A-40 | H | H |
| 4882 | 0 | A-41 | H | m-D13 | A-41 | H | H |
| 4883 | 0 | A-42 | H | m-D13 | A-42 | H | H |
| 4884 | 0 | A-43 | H | m-D13 | A-43 | H | H |
| 4885 | 0 | A-44 | H | m-D13 | A-44 | H | H |
| 4886 | 0 | A-45 | H | m-D13 | A-45 | H | H |
| 4887 | 0 | A-46 | H | m-D13 | A-46 | H | H |
| 4888 | 0 | A-47 | H | m-D13 | A-47 | H | H |
| 4889 | 0 | A-1 | H | m-D14 | A-1 | H | H |
| 4890 | 0 | A-2 | H | m-D14 | A-2 | H | H |
| 4891 | 0 | A-3 | H | m-D14 | A-3 | H | H |
| 4892 | 0 | A-4 | H | m-D14 | A-4 | H | H |
| 4893 | 0 | A-5 | H | m-D14 | A-5 | H | H |
| 4894 | 0 | A-6 | H | m-D14 | A-6 | H | H |
| 4895 | 0 | A-7 | H | m-D14 | A-7 | H | H |
| 4896 | 0 | A-8 | H | m-D14 | A-8 | H | H |
| 4897 | 0 | A-9 | H | m-D14 | A-9 | H | H |
| 4898 | 0 | A-10 | H | m-D14 | A-10 | H | H |
| 4899 | 0 | A-11 | H | m-D14 | A-11 | H | H |
| 4900 | 0 | A-12 | H | m-D14 | A-12 | H | H |
| 4901 | 0 | A-13 | H | m-D14 | A-13 | H | H |
| 4902 | 0 | A-14 | H | m-D14 | A-14 | H | H |
| 4903 | 0 | A-15 | H | m-D14 | A-15 | H | H |
| 4904 | 0 | A-16 | H | m-D14 | A-16 | H | H |
| 4905 | 0 | A-17 | H | m-D14 | A-17 | H | H |
| 4906 | 0 | A-18 | H | m-D14 | A-18 | H | H |
| 4907 | 0 | A-19 | H | m-D14 | A-19 | H | H |
| 4908 | 0 | A-20 | H | m-D14 | A-20 | H | H |
| 4909 | 0 | A-21 | H | m-D14 | A-21 | H | H |
| 4910 | 0 | A-22 | H | m-D14 | A-22 | H | H |
| 4911 | 0 | A-23 | H | m-D14 | A-23 | H | H |
| 4912 | 0 | A-24 | H | m-D14 | A-24 | H | H |
| 4913 | 0 | A-25 | H | m-D14 | A-25 | H | H |
| 4914 | 0 | A-26 | H | m-D14 | A-26 | H | H |
| 4915 | 0 | A-27 | H | m-D14 | A-27 | H | H |
| 4916 | 0 | A-28 | H | m-D14 | A-28 | H | H |
| 4917 | 0 | A-29 | H | m-D14 | A-29 | H | H |
| 4918 | 0 | A-30 | H | m-D14 | A-30 | H | H |
| 4919 | 0 | A-31 | H | m-D14 | A-31 | H | H |
| 4920 | 0 | A-32 | H | m-D14 | A-32 | H | H |
| 4921 | 0 | A-33 | H | m-D14 | A-33 | H | H |
| 4922 | 0 | A-34 | H | m-D14 | A-34 | H | H |
| 4923 | 0 | A-35 | H | m-D14 | A-35 | H | H |
| 4924 | 0 | A-36 | H | m-D14 | A-36 | H | H |
| 4925 | 0 | A-37 | H | m-D14 | A-37 | H | H |
| 4926 | 0 | A-38 | H | m-D14 | A-38 | H | H |
| 4927 | 0 | A-39 | H | m-D14 | A-39 | H | H |
| 4928 | 0 | A-40 | H | m-D14 | A-40 | H | H |
| 4929 | 0 | A-41 | H | m-D14 | A-41 | H | H |
| 4930 | 0 | A-42 | H | m-D14 | A-42 | H | H |
| 4931 | 0 | A-43 | H | m-D14 | A-43 | H | H |
| 4932 | 0 | A-44 | H | m-D14 | A-44 | H | H |
| 4933 | 0 | A-45 | H | m-D14 | A-45 | H | H |
| 4934 | 0 | A-46 | H | m-D14 | A-46 | H | H |
| 4935 | 0 | A-47 | H | m-D14 | A-47 | H | H |
| 4936 | 0 | A-1 | H | m-D1 | m-D1 | H | H |
| 4937 | 0 | A-2 | H | m-D1 | m-D1 | H | H |
| 4938 | 0 | A-3 | H | m-D1 | m-D1 | H | H |
| 4939 | 0 | A-4 | H | m-D1 | m-D1 | H | H |
| 4940 | 0 | A-5 | H | m-D1 | m-D1 | H | H |
| 4941 | 0 | A-6 | H | m-D1 | m-D1 | H | H |
| 4942 | 0 | A-7 | H | m-D1 | m-D1 | H | H |
| 4943 | 0 | A-8 | H | m-D1 | m-D1 | H | H |
| 4944 | 0 | A-9 | H | m-D1 | m-D1 | H | H |
| 4945 | 0 | A-10 | H | m-D1 | m-D1 | H | H |
| 4946 | 0 | A-11 | H | m-D1 | m-D1 | H | H |
| 4947 | 0 | A-12 | H | m-D1 | m-D1 | H | H |
| 4948 | 0 | A-13 | H | m-D1 | m-D1 | H | H |
| 4949 | 0 | A-14 | H | m-D1 | m-D1 | H | H |
| 4950 | 0 | A-15 | H | m-D1 | m-D1 | H | H |
| 4951 | 0 | A-16 | H | m-D1 | m-D1 | H | H |
| 4952 | 0 | A-17 | H | m-D1 | m-D1 | H | H |
| 4953 | 0 | A-18 | H | m-D1 | m-D1 | H | H |
| 4954 | 0 | A-19 | H | m-D1 | m-D1 | H | H |
| 4955 | 0 | A-20 | H | m-D1 | m-D1 | H | H |
| 4956 | 0 | A-21 | H | m-D1 | m-D1 | H | H |
| 4957 | 0 | A-22 | H | m-D1 | m-D1 | H | H |
| 4958 | 0 | A-23 | H | m-D1 | m-D1 | H | H |
| 4959 | 0 | A-24 | H | m-D1 | m-D1 | H | H |
| 4960 | 0 | A-25 | H | m-D1 | m-D1 | H | H |
| 4961 | 0 | A-26 | H | m-D1 | m-D1 | H | H |
| 4962 | 0 | A-27 | H | m-D1 | m-D1 | H | H |
| 4963 | 0 | A-28 | H | m-D1 | m-D1 | H | H |
| 4964 | 0 | A-29 | H | m-D1 | m-D1 | H | H |
| 4965 | 0 | A-30 | H | m-D1 | m-D1 | H | H |
| 4966 | 0 | A-31 | H | m-D1 | m-D1 | H | H |
| 4967 | 0 | A-32 | H | m-D1 | m-D1 | H | H |
| 4968 | 0 | A-33 | H | m-D1 | m-D1 | H | H |
| 4969 | 0 | A-34 | H | m-D1 | m-D1 | H | H |
| 4970 | 0 | A-35 | H | m-D1 | m-D1 | H | H |
| 4971 | 0 | A-36 | H | m-D1 | m-D1 | H | H |
| 4972 | 0 | A-37 | H | m-D1 | m-D1 | H | H |
| 4973 | 0 | A-38 | H | m-D1 | m-D1 | H | H |
| 4974 | 0 | A-39 | H | m-D1 | m-D1 | H | H |
| 4975 | 0 | A-40 | H | m-D1 | m-D1 | H | H |
| 4976 | 0 | A-41 | H | m-D1 | m-D1 | H | H |
| 4977 | 0 | A-42 | H | m-D1 | m-D1 | H | H |
| 4978 | 0 | A-43 | H | m-D1 | m-D1 | H | H |
| 4979 | 0 | A-44 | H | m-D1 | m-D1 | H | H |
| 4980 | 0 | A-45 | H | m-D1 | m-D1 | H | H |
| 4981 | 0 | A-46 | H | m-D1 | m-D1 | H | H |
| 4982 | 0 | A-47 | H | m-D1 | m-D1 | H | H |
| 4983 | 0 | A-1 | H | m-D2 | m-D2 | H | H |
| 4984 | 0 | A-2 | H | m-D2 | m-D2 | H | H |
| 4985 | 0 | A-3 | H | m-D2 | m-D2 | H | H |
| 4986 | 0 | A-4 | H | m-D2 | m-D2 | H | H |
| 4987 | 0 | A-5 | H | m-D2 | m-D2 | H | H |
| 4988 | 0 | A-6 | H | m-D2 | m-D2 | H | H |
| 4989 | 0 | A-7 | H | m-D2 | m-D2 | H | H |
| 4990 | 0 | A-8 | H | m-D2 | m-D2 | H | H |
| 4991 | 0 | A-9 | H | m-D2 | m-D2 | H | H |
| 4992 | 0 | A-10 | H | m-D2 | m-D2 | H | H |
| 4993 | 0 | A-11 | H | m-D2 | m-D2 | H | H |
| 4994 | 0 | A-12 | H | m-D2 | m-D2 | H | H |
| 4995 | 0 | A-13 | H | m-D2 | m-D2 | H | H |
| 4996 | 0 | A-14 | H | m-D2 | m-D2 | H | H |
| 4997 | 0 | A-15 | H | m-D2 | m-D2 | H | H |
| 4998 | 0 | A-16 | H | m-D2 | m-D2 | H | H |
| 4999 | 0 | A-17 | H | m-D2 | m-D2 | H | H |
| 5000 | 0 | A-18 | H | m-D2 | m-D2 | H | H |
| 5001 | 0 | A-19 | H | m-D2 | m-D2 | H | H |
| 5002 | 0 | A-20 | H | m-D2 | m-D2 | H | H |
| 5003 | 0 | A-21 | H | m-D2 | m-D2 | H | H |
| 5004 | 0 | A-22 | H | m-D2 | m-D2 | H | H |
| 5005 | 0 | A-23 | H | m-D2 | m-D2 | H | H |
| 5006 | 0 | A-24 | H | m-D2 | m-D2 | H | H |
| 5007 | 0 | A-25 | H | m-D2 | m-D2 | H | H |
| 5008 | 0 | A-26 | H | m-D2 | m-D2 | H | H |
| 5009 | 0 | A-27 | H | m-D2 | m-D2 | H | H |
| 5010 | 0 | A-28 | H | m-D2 | m-D2 | H | H |
| 5011 | 0 | A-29 | H | m-D2 | m-D2 | H | H |
| 5012 | 0 | A-30 | H | m-D2 | m-D2 | H | H |
| 5013 | 0 | A-31 | H | m-D2 | m-D2 | H | H |
| 5014 | 0 | A-32 | H | m-D2 | m-D2 | H | H |
| 5015 | 0 | A-33 | H | m-D2 | m-D2 | H | H |
| 5016 | 0 | A-34 | H | m-D2 | m-D2 | H | H |
| 5017 | 0 | A-35 | H | m-D2 | m-D2 | H | H |
| 5018 | 0 | A-36 | H | m-D2 | m-D2 | H | H |
| 5019 | 0 | A-37 | H | m-D2 | m-D2 | H | H |
| 5020 | 0 | A-38 | H | m-D2 | m-D2 | H | H |
| 5021 | 0 | A-39 | H | m-D2 | m-D2 | H | H |
| 5022 | 0 | A-40 | H | m-D2 | m-D2 | H | H |
| 5023 | 0 | A-41 | H | m-D2 | m-D2 | H | H |
| 5024 | 0 | A-42 | H | m-D2 | m-D2 | H | H |
| 5025 | 0 | A-43 | H | m-D2 | m-D2 | H | H |
| 5026 | 0 | A-44 | H | m-D2 | m-D2 | H | H |
| 5027 | 0 | A-45 | H | m-D2 | m-D2 | H | H |
| 5028 | 0 | A-46 | H | m-D2 | m-D2 | H | H |
| 5029 | 0 | A-47 | H | m-D2 | m-D2 | H | H |
| 5030 | 0 | A-1 | H | m-D3 | m-D3 | H | H |
| 5031 | 0 | A-2 | H | m-D3 | m-D3 | H | H |
| 5032 | 0 | A-3 | H | m-D3 | m-D3 | H | H |
| 5033 | 0 | A-4 | H | m-D3 | m-D3 | H | H |
| 5034 | 0 | A-5 | H | m-D3 | m-D3 | H | H |
| 5035 | 0 | A-6 | H | m-D3 | m-D3 | H | H |
| 5036 | 0 | A-7 | H | m-D3 | m-D3 | H | H |
| 5037 | 0 | A-8 | H | m-D3 | m-D3 | H | H |
| 5038 | 0 | A-9 | H | m-D3 | m-D3 | H | H |
| 5039 | 0 | A-10 | H | m-D3 | m-D3 | H | H |
| 5040 | 0 | A-11 | H | m-D3 | m-D3 | H | H |
| 5041 | 0 | A-12 | H | m-D3 | m-D3 | H | H |
| 5042 | 0 | A-13 | H | m-D3 | m-D3 | H | H |
| 5043 | 0 | A-14 | H | m-D3 | m-D3 | H | H |
| 5044 | 0 | A-15 | H | m-D3 | m-D3 | H | H |
| 5045 | 0 | A-16 | H | m-D3 | m-D3 | H | H |
| 5046 | 0 | A-17 | H | m-D3 | m-D3 | H | H |
| 5047 | 0 | A-18 | H | m-D3 | m-D3 | H | H |
| 5048 | 0 | A-19 | H | m-D3 | m-D3 | H | H |
| 5049 | 0 | A-20 | H | m-D3 | m-D3 | H | H |
| 5050 | 0 | A-21 | H | m-D3 | m-D3 | H | H |
| 5051 | 0 | A-22 | H | m-D3 | m-D3 | H | H |
| 5052 | 0 | A-23 | H | m-D3 | m-D3 | H | H |
| 5053 | 0 | A-24 | H | m-D3 | m-D3 | H | H |
| 5054 | 0 | A-25 | H | m-D3 | m-D3 | H | H |
| 5055 | 0 | A-26 | H | m-D3 | m-D3 | H | H |
| 5056 | 0 | A-27 | H | m-D3 | m-D3 | H | H |
| 5057 | 0 | A-28 | H | m-D3 | m-D3 | H | H |
| 5058 | 0 | A-29 | H | m-D3 | m-D3 | H | H |
| 5059 | 0 | A-30 | H | m-D3 | m-D3 | H | H |
| 5060 | 0 | A-31 | H | m-D3 | m-D3 | H | H |
| 5061 | 0 | A-32 | H | m-D3 | m-D3 | H | H |
| 5062 | 0 | A-33 | H | m-D3 | m-D3 | H | H |
| 5063 | 0 | A-34 | H | m-D3 | m-D3 | H | H |
| 5064 | 0 | A-35 | H | m-D3 | m-D3 | H | H |
| 5065 | 0 | A-36 | H | m-D3 | m-D3 | H | H |
| 5066 | 0 | A-37 | H | m-D3 | m-D3 | H | H |
| 5067 | 0 | A-38 | H | m-D3 | m-D3 | H | H |
| 5068 | 0 | A-39 | H | m-D3 | m-D3 | H | H |
| 5069 | 0 | A-40 | H | m-D3 | m-D3 | H | H |
| 5070 | 0 | A-41 | H | m-D3 | m-D3 | H | H |
| 5071 | 0 | A-42 | H | m-D3 | m-D3 | H | H |
| 5072 | 0 | A-43 | H | m-D3 | m-D3 | H | H |
| 5073 | 0 | A-44 | H | m-D3 | m-D3 | H | H |
| 5074 | 0 | A-45 | H | m-D3 | m-D3 | H | H |
| 5075 | 0 | A-46 | H | m-D3 | m-D3 | H | H |
| 5076 | 0 | A-47 | H | m-D3 | m-D3 | H | H |
| 5077 | 0 | A-1 | H | m-D5 | m-D5 | H | H |
| 5078 | 0 | A-2 | H | m-D5 | m-D5 | H | H |
| 5079 | 0 | A-3 | H | m-D5 | m-D5 | H | H |
| 5080 | 0 | A-4 | H | m-D5 | m-D5 | H | H |
| 5081 | 0 | A-5 | H | m-D5 | m-D5 | H | H |
| 5082 | 0 | A-6 | H | m-D5 | m-D5 | H | H |
| 5083 | 0 | A-7 | H | m-D5 | m-D5 | H | H |
| 5084 | 0 | A-8 | H | m-D5 | m-D5 | H | H |
| 5085 | 0 | A-9 | H | m-D5 | m-D5 | H | H |
| 5086 | 0 | A-10 | H | m-D5 | m-D5 | H | H |
| 5087 | 0 | A-11 | H | m-D5 | m-D5 | H | H |
| 5088 | 0 | A-12 | H | m-D5 | m-D5 | H | H |
| 5089 | 0 | A-13 | H | m-D5 | m-D5 | H | H |
| 5090 | 0 | A-14 | H | m-D5 | m-D5 | H | H |
| 5091 | 0 | A-15 | H | m-D5 | m-D5 | H | H |
| 5092 | 0 | A-16 | H | m-D5 | m-D5 | H | H |
| 5093 | 0 | A-17 | H | m-D5 | m-D5 | H | H |
| 5094 | 0 | A-18 | H | m-D5 | m-D5 | H | H |
| 5095 | 0 | A-19 | H | m-D5 | m-D5 | H | H |
| 5096 | 0 | A-20 | H | m-D5 | m-D5 | H | H |
| 5097 | 0 | A-21 | H | m-D5 | m-D5 | H | H |
| 5098 | 0 | A-22 | H | m-D5 | m-D5 | H | H |
| 5099 | 0 | A-23 | H | m-D5 | m-D5 | H | H |
| 5100 | 0 | A-24 | H | m-D5 | m-D5 | H | H |
| 5101 | 0 | A-25 | H | m-D5 | m-D5 | H | H |
| 5102 | 0 | A-26 | H | m-D5 | m-D5 | H | H |
| 5103 | 0 | A-27 | H | m-D5 | m-D5 | H | H |
| 5104 | 0 | A-28 | H | m-D5 | m-D5 | H | H |
| 5105 | 0 | A-29 | H | m-D5 | m-D5 | H | H |
| 5106 | 0 | A-30 | H | m-D5 | m-D5 | H | H |
| 5107 | 0 | A-31 | H | m-D5 | m-D5 | H | H |
| 5108 | 0 | A-32 | H | m-D5 | m-D5 | H | H |
| 5109 | 0 | A-33 | H | m-D5 | m-D5 | H | H |
| 5110 | 0 | A-34 | H | m-D5 | m-D5 | H | H |
| 5111 | 0 | A-35 | H | m-D5 | m-D5 | H | H |
| 5112 | 0 | A-36 | H | m-D5 | m-D5 | H | H |
| 5113 | 0 | A-37 | H | m-D5 | m-D5 | H | H |
| 5114 | 0 | A-38 | H | m-D5 | m-D5 | H | H |
| 5115 | 0 | A-39 | H | m-D5 | m-D5 | H | H |
| 5116 | 0 | A-40 | H | m-D5 | m-D5 | H | H |
| 5117 | 0 | A-41 | H | m-D5 | m-D5 | H | H |
| 5118 | 0 | A-42 | H | m-D5 | m-D5 | H | H |
| 5119 | 0 | A-43 | H | m-D5 | m-D5 | H | H |
| 5120 | 0 | A-44 | H | m-D5 | m-D5 | H | H |
| 5121 | 0 | A-45 | H | m-D5 | m-D5 | H | H |
| 5122 | 0 | A-46 | H | m-D5 | m-D5 | H | H |
| 5123 | 0 | A-47 | H | m-D5 | m-D5 | H | H |
| 5124 | 0 | A-1 | H | m-D9 | m-D9 | H | H |
| 5125 | 0 | A-2 | H | m-D9 | m-D9 | H | H |
| 5126 | 0 | A-3 | H | m-D9 | m-D9 | H | H |
| 5127 | 0 | A-4 | H | m-D9 | m-D9 | H | H |
| 5128 | 0 | A-5 | H | m-D9 | m-D9 | H | H |
| 5129 | 0 | A-6 | H | m-D9 | m-D9 | H | H |
| 5130 | 0 | A-7 | H | m-D9 | m-D9 | H | H |
| 5131 | 0 | A-8 | H | m-D9 | m-D9 | H | H |
| 5132 | 0 | A-9 | H | m-D9 | m-D9 | H | H |
| 5133 | 0 | A-10 | H | m-D9 | m-D9 | H | H |
| 5134 | 0 | A-11 | H | m-D9 | m-D9 | H | H |
| 5135 | 0 | A-12 | H | m-D9 | m-D9 | H | H |
| 5136 | 0 | A-13 | H | m-D9 | m-D9 | H | H |
| 5137 | 0 | A-14 | H | m-D9 | m-D9 | H | H |
| 5138 | 0 | A-15 | H | m-D9 | m-D9 | H | H |
| 5139 | 0 | A-16 | H | m-D9 | m-D9 | H | H |
| 5140 | 0 | A-17 | H | m-D9 | m-D9 | H | H |
| 5141 | 0 | A-18 | H | m-D9 | m-D9 | H | H |
| 5142 | 0 | A-19 | H | m-D9 | m-D9 | H | H |
| 5143 | 0 | A-20 | H | m-D9 | m-D9 | H | H |
| 5144 | 0 | A-21 | H | m-D9 | m-D9 | H | H |
| 5145 | 0 | A-22 | H | m-D9 | m-D9 | H | H |
| 5146 | 0 | A-23 | H | m-D9 | m-D9 | H | H |
| 5147 | 0 | A-24 | H | m-D9 | m-D9 | H | H |
| 5148 | 0 | A-25 | H | m-D9 | m-D9 | H | H |
| 5149 | 0 | A-26 | H | m-D9 | m-D9 | H | H |
| 5150 | 0 | A-27 | H | m-D9 | m-D9 | H | H |
| 5151 | 0 | A-28 | H | m-D9 | m-D9 | H | H |
| 5152 | 0 | A-29 | H | m-D9 | m-D9 | H | H |
| 5153 | 0 | A-30 | H | m-D9 | m-D9 | H | H |
| 5154 | 0 | A-31 | H | m-D9 | m-D9 | H | H |
| 5155 | 0 | A-32 | H | m-D9 | m-D9 | H | H |
| 5156 | 0 | A-33 | H | m-D9 | m-D9 | H | H |
| 5157 | 0 | A-34 | H | m-D9 | m-D9 | H | H |
| 5158 | 0 | A-35 | H | m-D9 | m-D9 | H | H |
| 5159 | 0 | A-36 | H | m-D9 | m-D9 | H | H |
| 5160 | 0 | A-37 | H | m-D9 | m-D9 | H | H |
| 5161 | 0 | A-38 | H | m-D9 | m-D9 | H | H |
| 5162 | 0 | A-39 | H | m-D9 | m-D9 | H | H |
| 5163 | 0 | A-40 | H | m-D9 | m-D9 | H | H |
| 5164 | 0 | A-41 | H | m-D9 | m-D9 | H | H |
| 5165 | 0 | A-42 | H | m-D9 | m-D9 | H | H |
| 5166 | 0 | A-43 | H | m-D9 | m-D9 | H | H |
| 5167 | 0 | A-44 | H | m-D9 | m-D9 | H | H |
| 5168 | 0 | A-45 | H | m-D9 | m-D9 | H | H |
| 5169 | 0 | A-46 | H | m-D9 | m-D9 | H | H |
| 5170 | 0 | A-47 | H | m-D9 | m-D9 | H | H |
| 5171 | 0 | A-1 | H | m-D11 | m-D11 | H | H |
| 5172 | 0 | A-2 | H | m-D11 | m-D11 | H | H |
| 5173 | 0 | A-3 | H | m-D11 | m-D11 | H | H |
| 5174 | 0 | A-4 | H | m-D11 | m-D11 | H | H |
| 5175 | 0 | A-5 | H | m-D11 | m-D11 | H | H |
| 5176 | 0 | A-6 | H | m-D11 | m-D11 | H | H |
| 5177 | 0 | A-7 | H | m-D11 | m-D11 | H | H |
| 5178 | 0 | A-8 | H | m-D11 | m-D11 | H | H |
| 5179 | 0 | A-9 | H | m-D11 | m-D11 | H | H |
| 5180 | 0 | A-10 | H | m-D11 | m-D11 | H | H |
| 5181 | 0 | A-11 | H | m-D11 | m-D11 | H | H |
| 5182 | 0 | A-12 | H | m-D11 | m-D11 | H | H |
| 5183 | 0 | A-13 | H | m-D11 | m-D11 | H | H |
| 5184 | 0 | A-14 | H | m-D11 | m-D11 | H | H |
| 5185 | 0 | A-15 | H | m-D11 | m-D11 | H | H |
| 5186 | 0 | A-16 | H | m-D11 | m-D11 | H | H |
| 5187 | 0 | A-17 | H | m-D11 | m-D11 | H | H |
| 5188 | 0 | A-18 | H | m-D11 | m-D11 | H | H |
| 5189 | 0 | A-19 | H | m-D11 | m-D11 | H | H |
| 5190 | 0 | A-20 | H | m-D11 | m-D11 | H | H |
| 5191 | 0 | A-21 | H | m-D11 | m-D11 | H | H |
| 5192 | 0 | A-22 | H | m-D11 | m-D11 | H | H |
| 5193 | 0 | A-23 | H | m-D11 | m-D11 | H | H |
| 5194 | 0 | A-24 | H | m-D11 | m-D11 | H | H |
| 5195 | 0 | A-25 | H | m-D11 | m-D11 | H | H |
| 5196 | 0 | A-26 | H | m-D11 | m-D11 | H | H |
| 5197 | 0 | A-27 | H | m-D11 | m-D11 | H | H |
| 5198 | 0 | A-28 | H | m-D11 | m-D11 | H | H |
| 5199 | 0 | A-29 | H | m-D11 | m-D11 | H | H |
| 5200 | 0 | A-30 | H | m-D11 | m-D11 | H | H |
| 5201 | 0 | A-31 | H | m-D11 | m-D11 | H | H |
| 5202 | 0 | A-32 | H | m-D11 | m-D11 | H | H |
| 5203 | 0 | A-33 | H | m-D11 | m-D11 | H | H |
| 5204 | 0 | A-34 | H | m-D11 | m-D11 | H | H |
| 5205 | 0 | A-35 | H | m-D11 | m-D11 | H | H |
| 5206 | 0 | A-36 | H | m-D11 | m-D11 | H | H |
| 5207 | 0 | A-37 | H | m-D11 | m-D11 | H | H |
| 5208 | 0 | A-38 | H | m-D11 | m-D11 | H | H |
| 5209 | 0 | A-39 | H | m-D11 | m-D11 | H | H |
| 5210 | 0 | A-40 | H | m-D11 | m-D11 | H | H |
| 5211 | 0 | A-41 | H | m-D11 | m-D11 | H | H |
| 5212 | 0 | A-42 | H | m-D11 | m-D11 | H | H |
| 5213 | 0 | A-43 | H | m-D11 | m-D11 | H | H |
| 5214 | 0 | A-44 | H | m-D11 | m-D11 | H | H |
| 5215 | 0 | A-45 | H | m-D11 | m-D11 | H | H |
| 5216 | 0 | A-46 | H | m-D11 | m-D11 | H | H |
| 5217 | 0 | A-47 | H | m-D11 | m-D11 | H | H |
| 5218 | 0 | A-1 | H | m-D12 | m-D12 | H | H |
| 5219 | 0 | A-2 | H | m-D12 | m-D12 | H | H |
| 5220 | 0 | A-3 | H | m-D12 | m-D12 | H | H |
| 5221 | 0 | A-4 | H | m-D12 | m-D12 | H | H |
| 5222 | 0 | A-5 | H | m-D12 | m-D12 | H | H |
| 5223 | 0 | A-6 | H | m-D12 | m-D12 | H | H |
| 5224 | 0 | A-7 | H | m-D12 | m-D12 | H | H |
| 5225 | 0 | A-8 | H | m-D12 | m-D12 | H | H |
| 5226 | 0 | A-9 | H | m-D12 | m-D12 | H | H |
| 5227 | 0 | A-10 | H | m-D12 | m-D12 | H | H |
| 5228 | 0 | A-11 | H | m-D12 | m-D12 | H | H |
| 5229 | 0 | A-12 | H | m-D12 | m-D12 | H | H |
| 5230 | 0 | A-13 | H | m-D12 | m-D12 | H | H |
| 5231 | 0 | A-14 | H | m-D12 | m-D12 | H | H |
| 5232 | 0 | A-15 | H | m-D12 | m-D12 | H | H |
| 5233 | 0 | A-16 | H | m-D12 | m-D12 | H | H |
| 5234 | 0 | A-17 | H | m-D12 | m-D12 | H | H |
| 5235 | 0 | A-18 | H | m-D12 | m-D12 | H | H |
| 5236 | 0 | A-19 | H | m-D12 | m-D12 | H | H |
| 5237 | 0 | A-20 | H | m-D12 | m-D12 | H | H |
| 5238 | 0 | A-21 | H | m-D12 | m-D12 | H | H |
| 5239 | 0 | A-22 | H | m-D12 | m-D12 | H | H |
| 5240 | 0 | A-23 | H | m-D12 | m-D12 | H | H |
| 5241 | 0 | A-24 | H | m-D12 | m-D12 | H | H |
| 5242 | 0 | A-25 | H | m-D12 | m-D12 | H | H |
| 5243 | 0 | A-26 | H | m-D12 | m-D12 | H | H |
| 5244 | 0 | A-27 | H | m-D12 | m-D12 | H | H |
| 5245 | 0 | A-28 | H | m-D12 | m-D12 | H | H |
| 5246 | 0 | A-29 | H | m-D12 | m-D12 | H | H |
| 5247 | 0 | A-30 | H | m-D12 | m-D12 | H | H |
| 5248 | 0 | A-31 | H | m-D12 | m-D12 | H | H |
| 5249 | 0 | A-32 | H | m-D12 | m-D12 | H | H |
| 5250 | 0 | A-33 | H | m-D12 | m-D12 | H | H |
| 5251 | 0 | A-34 | H | m-D12 | m-D12 | H | H |
| 5252 | 0 | A-35 | H | m-D12 | m-D12 | H | H |
| 5253 | 0 | A-36 | H | m-D12 | m-D12 | H | H |
| 5254 | 0 | A-37 | H | m-D12 | m-D12 | H | H |
| 5255 | 0 | A-38 | H | m-D12 | m-D12 | H | H |
| 5256 | 0 | A-39 | H | m-D12 | m-D12 | H | H |
| 5257 | 0 | A-40 | H | m-D12 | m-D12 | H | H |
| 5258 | 0 | A-41 | H | m-D12 | m-D12 | H | H |
| 5259 | 0 | A-42 | H | m-D12 | m-D12 | H | H |
| 5260 | 0 | A-43 | H | m-D12 | m-D12 | H | H |
| 5261 | 0 | A-44 | H | m-D12 | m-D12 | H | H |
| 5262 | 0 | A-45 | H | m-D12 | m-D12 | H | H |
| 5263 | 0 | A-46 | H | m-D12 | m-D12 | H | H |
| 5264 | 0 | A-47 | H | m-D12 | m-D12 | H | H |
| 5265 | 0 | A-1 | H | m-D13 | m-D13 | H | H |
| 5266 | 0 | A-2 | H | m-D13 | m-D13 | H | H |
| 5267 | 0 | A-3 | H | m-D13 | m-D13 | H | H |
| 5268 | 0 | A-4 | H | m-D13 | m-D13 | H | H |
| 5269 | 0 | A-5 | H | m-D13 | m-D13 | H | H |
| 5270 | 0 | A-6 | H | m-D13 | m-D13 | H | H |
| 5271 | 0 | A-7 | H | m-D13 | m-D13 | H | H |
| 5272 | 0 | A-8 | H | m-D13 | m-D13 | H | H |
| 5273 | 0 | A-9 | H | m-D13 | m-D13 | H | H |
| 5274 | 0 | A-10 | H | m-D13 | m-D13 | H | H |
| 5275 | 0 | A-11 | H | m-D13 | m-D13 | H | H |
| 5276 | 0 | A-12 | H | m-D13 | m-D13 | H | H |
| 5277 | 0 | A-13 | H | m-D13 | m-D13 | H | H |
| 5278 | 0 | A-14 | H | m-D13 | m-D13 | H | H |
| 5279 | 0 | A-15 | H | m-D13 | m-D13 | H | H |
| 5280 | 0 | A-16 | H | m-D13 | m-D13 | H | H |
| 5281 | 0 | A-17 | H | m-D13 | m-D13 | H | H |
| 5282 | 0 | A-18 | H | m-D13 | m-D13 | H | H |
| 5283 | 0 | A-19 | H | m-D13 | m-D13 | H | H |
| 5284 | 0 | A-20 | H | m-D13 | m-D13 | H | H |
| 5285 | 0 | A-21 | H | m-D13 | m-D13 | H | H |
| 5286 | 0 | A-22 | H | m-D13 | m-D13 | H | H |
| 5287 | 0 | A-23 | H | m-D13 | m-D13 | H | H |
| 5288 | 0 | A-24 | H | m-D13 | m-D13 | H | H |
| 5289 | 0 | A-25 | H | m-D13 | m-D13 | H | H |
| 5290 | 0 | A-26 | H | m-D13 | m-D13 | H | H |
| 5291 | 0 | A-27 | H | m-D13 | m-D13 | H | H |
| 5292 | 0 | A-28 | H | m-D13 | m-D13 | H | H |
| 5293 | 0 | A-29 | H | m-D13 | m-D13 | H | H |
| 5294 | 0 | A-30 | H | m-D13 | m-D13 | H | H |
| 5295 | 0 | A-31 | H | m-D13 | m-D13 | H | H |
| 5296 | 0 | A-32 | H | m-D13 | m-D13 | H | H |
| 5297 | 0 | A-33 | H | m-D13 | m-D13 | H | H |
| 5298 | 0 | A-34 | H | m-D13 | m-D13 | H | H |
| 5299 | 0 | A-35 | H | m-D13 | m-D13 | H | H |
| 5300 | 0 | A-36 | H | m-D13 | m-D13 | H | H |
| 5301 | 0 | A-37 | H | m-D13 | m-D13 | H | H |
| 5302 | 0 | A-38 | H | m-D13 | m-D13 | H | H |
| 5303 | 0 | A-39 | H | m-D13 | m-D13 | H | H |
| 5304 | 0 | A-40 | H | m-D13 | m-D13 | H | H |
| 5305 | 0 | A-41 | H | m-D13 | m-D13 | H | H |
| 5306 | 0 | A-42 | H | m-D13 | m-D13 | H | H |
| 5307 | 0 | A-43 | H | m-D13 | m-D13 | H | H |
| 5308 | 0 | A-44 | H | m-D13 | m-D13 | H | H |
| 5309 | 0 | A-45 | H | m-D13 | m-D13 | H | H |
| 5310 | 0 | A-46 | H | m-D13 | m-D13 | H | H |
| 5311 | 0 | A-47 | H | m-D13 | m-D13 | H | H |
| 5312 | 0 | A-1 | H | m-D14 | m-D14 | H | H |
| 5313 | 0 | A-2 | H | m-D14 | m-D14 | H | H |
| 5314 | 0 | A-3 | H | m-D14 | m-D14 | H | H |
| 5315 | 0 | A-4 | H | m-D14 | m-D14 | H | H |
| 5316 | 0 | A-5 | H | m-D14 | m-D14 | H | H |
| 5317 | 0 | A-6 | H | m-D14 | m-D14 | H | H |
| 5318 | 0 | A-7 | H | m-D14 | m-D14 | H | H |
| 5319 | 0 | A-8 | H | m-D14 | m-D14 | H | H |
| 5320 | 0 | A-9 | H | m-D14 | m-D14 | H | H |
| 5321 | 0 | A-10 | H | m-D14 | m-D14 | H | H |
| 5322 | 0 | A-11 | H | m-D14 | m-D14 | H | H |
| 5323 | 0 | A-12 | H | m-D14 | m-D14 | H | H |
| 5324 | 0 | A-13 | H | m-D14 | m-D14 | H | H |
| 5325 | 0 | A-14 | H | m-D14 | m-D14 | H | H |
| 5326 | 0 | A-15 | H | m-D14 | m-D14 | H | H |
| 5327 | 0 | A-16 | H | m-D14 | m-D14 | H | H |
| 5328 | 0 | A-17 | H | m-D14 | m-D14 | H | H |
| 5329 | 0 | A-18 | H | m-D14 | m-D14 | H | H |
| 5330 | 0 | A-19 | H | m-D14 | m-D14 | H | H |
| 5331 | 0 | A-20 | H | m-D14 | m-D14 | H | H |
| 5332 | 0 | A-21 | H | m-D14 | m-D14 | H | H |
| 5333 | 0 | A-22 | H | m-D14 | m-D14 | H | H |
| 5334 | 0 | A-23 | H | m-D14 | m-D14 | H | H |
| 5335 | 0 | A-24 | H | m-D14 | m-D14 | H | H |
| 5336 | 0 | A-25 | H | m-D14 | m-D14 | H | H |
| 5337 | 0 | A-26 | H | m-D14 | m-D14 | H | H |
| 5338 | 0 | A-27 | H | m-D14 | m-D14 | H | H |
| 5339 | 0 | A-28 | H | m-D14 | m-D14 | H | H |
| 5340 | 0 | A-29 | H | m-D14 | m-D14 | H | H |
| 5341 | 0 | A-30 | H | m-D14 | m-D14 | H | H |
| 5342 | 0 | A-31 | H | m-D14 | m-D14 | H | H |
| 5343 | 0 | A-32 | H | m-D14 | m-D14 | H | H |
| 5344 | 0 | A-33 | H | m-D14 | m-D14 | H | H |
| 5345 | 0 | A-34 | H | m-D14 | m-D14 | H | H |
| 5346 | 0 | A-35 | H | m-D14 | m-D14 | H | H |
| 5347 | 0 | A-36 | H | m-D14 | m-D14 | H | H |
| 5348 | 0 | A-37 | H | m-D14 | m-D14 | H | H |
| 5349 | 0 | A-38 | H | m-D14 | m-D14 | H | H |
| 5350 | 0 | A-39 | H | m-D14 | m-D14 | H | H |
| 5351 | 0 | A-40 | H | m-D14 | m-D14 | H | H |
| 5352 | 0 | A-41 | H | m-D14 | m-D14 | H | H |
| 5353 | 0 | A-42 | H | m-D14 | m-D14 | H | H |
| 5354 | 0 | A-43 | H | m-D14 | m-D14 | H | H |
| 5355 | 0 | A-44 | H | m-D14 | m-D14 | H | H |
| 5356 | 0 | A-45 | H | m-D14 | m-D14 | H | H |
| 5357 | 0 | A-46 | H | m-D14 | m-D14 | H | H |
| 5358 | 0 | A-47 | H | m-D14 | m-D14 | H | H |
| 5359 | 0 | A-1 | m-D1 | m-D1 | H | H | H |
| 5360 | 0 | A-2 | m-D1 | m-D1 | H | H | H |
| 5361 | 0 | A-3 | m-D1 | m-D1 | H | H | H |
| 5362 | 0 | A-4 | m-D1 | m-D1 | H | H | H |
| 5363 | 0 | A-5 | m-D1 | m-D1 | H | H | H |
| 5364 | 0 | A-6 | m-D1 | m-D1 | H | H | H |
| 5365 | 0 | A-7 | m-D1 | m-D1 | H | H | H |
| 5366 | 0 | A-8 | m-D1 | m-D1 | H | H | H |
| 5367 | 0 | A-9 | m-D1 | m-D1 | H | H | H |
| 5368 | 0 | A-10 | m-D1 | m-D1 | H | H | H |
| 5369 | 0 | A-11 | m-D1 | m-D1 | H | H | H |
| 5370 | 0 | A-12 | m-D1 | m-D1 | H | H | H |
| 5371 | 0 | A-13 | m-D1 | m-D1 | H | H | H |
| 5372 | 0 | A-14 | m-D1 | m-D1 | H | H | H |
| 5373 | 0 | A-15 | m-D1 | m-D1 | H | H | H |
| 5374 | 0 | A-16 | m-D1 | m-D1 | H | H | H |
| 5375 | 0 | A-17 | m-D1 | m-D1 | H | H | H |
| 5376 | 0 | A-18 | m-D1 | m-D1 | H | H | H |
| 5377 | 0 | A-19 | m-D1 | m-D1 | H | H | H |
| 5378 | 0 | A-20 | m-D1 | m-D1 | H | H | H |
| 5379 | 0 | A-21 | m-D1 | m-D1 | H | H | H |
| 5380 | 0 | A-22 | m-D1 | m-D1 | H | H | H |
| 5381 | 0 | A-23 | m-D1 | m-D1 | H | H | H |
| 5382 | 0 | A-24 | m-D1 | m-D1 | H | H | H |
| 5383 | 0 | A-25 | m-D1 | m-D1 | H | H | H |
| 5384 | 0 | A-26 | m-D1 | m-D1 | H | H | H |
| 5385 | 0 | A-27 | m-D1 | m-D1 | H | H | H |
| 5386 | 0 | A-28 | m-D1 | m-D1 | H | H | H |
| 5387 | 0 | A-29 | m-D1 | m-D1 | H | H | H |
| 5388 | 0 | A-30 | m-D1 | m-D1 | H | H | H |
| 5389 | 0 | A-31 | m-D1 | m-D1 | H | H | H |
| 5390 | 0 | A-32 | m-D1 | m-D1 | H | H | H |
| 5391 | 0 | A-33 | m-D1 | m-D1 | H | H | H |
| 5392 | 0 | A-34 | m-D1 | m-D1 | H | H | H |
| 5393 | 0 | A-35 | m-D1 | m-D1 | H | H | H |
| 5394 | 0 | A-36 | m-D1 | m-D1 | H | H | H |
| 5395 | 0 | A-37 | m-D1 | m-D1 | H | H | H |
| 5396 | 0 | A-38 | m-D1 | m-D1 | H | H | H |
| 5397 | 0 | A-39 | m-D1 | m-D1 | H | H | H |
| 5398 | 0 | A-40 | m-D1 | m-D1 | H | H | H |
| 5399 | 0 | A-41 | m-D1 | m-D1 | H | H | H |
| 5400 | 0 | A-42 | m-D1 | m-D1 | H | H | H |
| 5401 | 0 | A-43 | m-D1 | m-D1 | H | H | H |
| 5402 | 0 | A-44 | m-D1 | m-D1 | H | H | H |
| 5403 | 0 | A-45 | m-D1 | m-D1 | H | H | H |
| 5404 | 0 | A-46 | m-D1 | m-D1 | H | H | H |
| 5405 | 0 | A-47 | m-D1 | m-D1 | H | H | H |
| 5406 | 0 | A-1 | m-D2 | m-D2 | H | H | H |
| 5407 | 0 | A-2 | m-D2 | m-D2 | H | H | H |
| 5408 | 0 | A-3 | m-D2 | m-D2 | H | H | H |
| 5409 | 0 | A-4 | m-D2 | m-D2 | H | H | H |
| 5410 | 0 | A-5 | m-D2 | m-D2 | H | H | H |
| 5411 | 0 | A-6 | m-D2 | m-D2 | H | H | H |
| 5412 | 0 | A-7 | m-D2 | m-D2 | H | H | H |
| 5413 | 0 | A-8 | m-D2 | m-D2 | H | H | H |
| 5414 | 0 | A-9 | m-D2 | m-D2 | H | H | H |
| 5415 | 0 | A-10 | m-D2 | m-D2 | H | H | H |
| 5416 | 0 | A-11 | m-D2 | m-D2 | H | H | H |
| 5417 | 0 | A-12 | m-D2 | m-D2 | H | H | H |
| 5418 | 0 | A-13 | m-D2 | m-D2 | H | H | H |
| 5419 | 0 | A-14 | m-D2 | m-D2 | H | H | H |
| 5420 | 0 | A-15 | m-D2 | m-D2 | H | H | H |
| 5421 | 0 | A-16 | m-D2 | m-D2 | H | H | H |
| 5422 | 0 | A-17 | m-D2 | m-D2 | H | H | H |
| 5423 | 0 | A-18 | m-D2 | m-D2 | H | H | H |
| 5424 | 0 | A-19 | m-D2 | m-D2 | H | H | H |
| 5425 | 0 | A-20 | m-D2 | m-D2 | H | H | H |
| 5426 | 0 | A-21 | m-D2 | m-D2 | H | H | H |
| 5427 | 0 | A-22 | m-D2 | m-D2 | H | H | H |
| 5428 | 0 | A-23 | m-D2 | m-D2 | H | H | H |
| 5429 | 0 | A-24 | m-D2 | m-D2 | H | H | H |
| 5430 | 0 | A-25 | m-D2 | m-D2 | H | H | H |
| 5431 | 0 | A-26 | m-D2 | m-D2 | H | H | H |
| 5432 | 0 | A-27 | m-D2 | m-D2 | H | H | H |
| 5433 | 0 | A-28 | m-D2 | m-D2 | H | H | H |
| 5434 | 0 | A-29 | m-D2 | m-D2 | H | H | H |
| 5435 | 0 | A-30 | m-D2 | m-D2 | H | H | H |
| 5436 | 0 | A-31 | m-D2 | m-D2 | H | H | H |
| 5437 | 0 | A-32 | m-D2 | m-D2 | H | H | H |
| 5438 | 0 | A-33 | m-D2 | m-D2 | H | H | H |
| 5439 | 0 | A-34 | m-D2 | m-D2 | H | H | H |
| 5440 | 0 | A-35 | m-D2 | m-D2 | H | H | H |
| 5441 | 0 | A-36 | m-D2 | m-D2 | H | H | H |
| 5442 | 0 | A-37 | m-D2 | m-D2 | H | H | H |
| 5443 | 0 | A-38 | m-D2 | m-D2 | H | H | H |
| 5444 | 0 | A-39 | m-D2 | m-D2 | H | H | H |
| 5445 | 0 | A-40 | m-D2 | m-D2 | H | H | H |
| 5446 | 0 | A-41 | m-D2 | m-D2 | H | H | H |
| 5447 | 0 | A-42 | m-D2 | m-D2 | H | H | H |
| 5448 | 0 | A-43 | m-D2 | m-D2 | H | H | H |
| 5449 | 0 | A-44 | m-D2 | m-D2 | H | H | H |
| 5450 | 0 | A-45 | m-D2 | m-D2 | H | H | H |
| 5451 | 0 | A-46 | m-D2 | m-D2 | H | H | H |
| 5452 | 0 | A-47 | m-D2 | m-D2 | H | H | H |
| 5453 | 0 | A-1 | m-D3 | m-D3 | H | H | H |
| 5454 | 0 | A-2 | m-D3 | m-D3 | H | H | H |
| 5455 | 0 | A-3 | m-D3 | m-D3 | H | H | H |
| 5456 | 0 | A-4 | m-D3 | m-D3 | H | H | H |
| 5457 | 0 | A-5 | m-D3 | m-D3 | H | H | H |
| 5458 | 0 | A-6 | m-D3 | m-D3 | H | H | H |
| 5459 | 0 | A-7 | m-D3 | m-D3 | H | H | H |
| 5460 | 0 | A-8 | m-D3 | m-D3 | H | H | H |
| 5461 | 0 | A-9 | m-D3 | m-D3 | H | H | H |
| 5462 | 0 | A-10 | m-D3 | m-D3 | H | H | H |
| 5463 | 0 | A-11 | m-D3 | m-D3 | H | H | H |
| 5464 | 0 | A-12 | m-D3 | m-D3 | H | H | H |
| 5465 | 0 | A-13 | m-D3 | m-D3 | H | H | H |
| 5466 | 0 | A-14 | m-D3 | m-D3 | H | H | H |
| 5467 | 0 | A-15 | m-D3 | m-D3 | H | H | H |
| 5468 | 0 | A-16 | m-D3 | m-D3 | H | H | H |
| 5469 | 0 | A-17 | m-D3 | m-D3 | H | H | H |
| 5470 | 0 | A-18 | m-D3 | m-D3 | H | H | H |
| 5471 | 0 | A-19 | m-D3 | m-D3 | H | H | H |
| 5472 | 0 | A-20 | m-D3 | m-D3 | H | H | H |
| 5473 | 0 | A-21 | m-D3 | m-D3 | H | H | H |
| 5474 | 0 | A-22 | m-D3 | m-D3 | H | H | H |
| 5475 | 0 | A-23 | m-D3 | m-D3 | H | H | H |
| 5476 | 0 | A-24 | m-D3 | m-D3 | H | H | H |
| 5477 | 0 | A-25 | m-D3 | m-D3 | H | H | H |
| 5478 | 0 | A-26 | m-D3 | m-D3 | H | H | H |
| 5479 | 0 | A-27 | m-D3 | m-D3 | H | H | H |
| 5480 | 0 | A-28 | m-D3 | m-D3 | H | H | H |
| 5481 | 0 | A-29 | m-D3 | m-D3 | H | H | H |
| 5482 | 0 | A-30 | m-D3 | m-D3 | H | H | H |
| 5483 | 0 | A-31 | m-D3 | m-D3 | H | H | H |
| 5484 | 0 | A-32 | m-D3 | m-D3 | H | H | H |
| 5485 | 0 | A-33 | m-D3 | m-D3 | H | H | H |
| 5486 | 0 | A-34 | m-D3 | m-D3 | H | H | H |
| 5487 | 0 | A-35 | m-D3 | m-D3 | H | H | H |
| 5488 | 0 | A-36 | m-D3 | m-D3 | H | H | H |
| 5489 | 0 | A-37 | m-D3 | m-D3 | H | H | H |
| 5490 | 0 | A-38 | m-D3 | m-D3 | H | H | H |
| 5491 | 0 | A-39 | m-D3 | m-D3 | H | H | H |
| 5492 | 0 | A-40 | m-D3 | m-D3 | H | H | H |
| 5493 | 0 | A-41 | m-D3 | m-D3 | H | H | H |
| 5494 | 0 | A-42 | m-D3 | m-D3 | H | H | H |
| 5495 | 0 | A-43 | m-D3 | m-D3 | H | H | H |
| 5496 | 0 | A-44 | m-D3 | m-D3 | H | H | H |
| 5497 | 0 | A-45 | m-D3 | m-D3 | H | H | H |
| 5498 | 0 | A-46 | m-D3 | m-D3 | H | H | H |
| 5499 | 0 | A-47 | m-D3 | m-D3 | H | H | H |
| 5500 | 0 | A-1 | m-D5 | m-D5 | H | H | H |
| 5501 | 0 | A-2 | m-D5 | m-D5 | H | H | H |
| 5502 | 0 | A-3 | m-D5 | m-D5 | H | H | H |
| 5503 | 0 | A-4 | m-D5 | m-D5 | H | H | H |
| 5504 | 0 | A-5 | m-D5 | m-D5 | H | H | H |
| 5505 | 0 | A-6 | m-D5 | m-D5 | H | H | H |
| 5506 | 0 | A-7 | m-D5 | m-D5 | H | H | H |
| 5507 | 0 | A-8 | m-D5 | m-D5 | H | H | H |
| 5508 | 0 | A-9 | m-D5 | m-D5 | H | H | H |
| 5509 | 0 | A-10 | m-D5 | m-D5 | H | H | H |
| 5510 | 0 | A-11 | m-D5 | m-D5 | H | H | H |
| 5511 | 0 | A-12 | m-D5 | m-D5 | H | H | H |
| 5512 | 0 | A-13 | m-D5 | m-D5 | H | H | H |
| 5513 | 0 | A-14 | m-D5 | m-D5 | H | H | H |
| 5514 | 0 | A-15 | m-D5 | m-D5 | H | H | H |
| 5515 | 0 | A-16 | m-D5 | m-D5 | H | H | H |
| 5516 | 0 | A-17 | m-D5 | m-D5 | H | H | H |
| 5517 | 0 | A-18 | m-D5 | m-D5 | H | H | H |
| 5518 | 0 | A-19 | m-D5 | m-D5 | H | H | H |
| 5519 | 0 | A-20 | m-D5 | m-D5 | H | H | H |
| 5520 | 0 | A-21 | m-D5 | m-D5 | H | H | H |
| 5521 | 0 | A-22 | m-D5 | m-D5 | H | H | H |
| 5522 | 0 | A-23 | m-D5 | m-D5 | H | H | H |
| 5523 | 0 | A-24 | m-D5 | m-D5 | H | H | H |
| 5524 | 0 | A-25 | m-D5 | m-D5 | H | H | H |
| 5525 | 0 | A-26 | m-D5 | m-D5 | H | H | H |
| 5526 | 0 | A-27 | m-D5 | m-D5 | H | H | H |
| 5527 | 0 | A-28 | m-D5 | m-D5 | H | H | H |
| 5528 | 0 | A-29 | m-D5 | m-D5 | H | H | H |
| 5529 | 0 | A-30 | m-D5 | m-D5 | H | H | H |
| 5530 | 0 | A-31 | m-D5 | m-D5 | H | H | H |
| 5531 | 0 | A-32 | m-D5 | m-D5 | H | H | H |
| 5532 | 0 | A-33 | m-D5 | m-D5 | H | H | H |
| 5533 | 0 | A-34 | m-D5 | m-D5 | H | H | H |
| 5534 | 0 | A-35 | m-D5 | m-D5 | H | H | H |
| 5535 | 0 | A-36 | m-D5 | m-D5 | H | H | H |
| 5536 | 0 | A-37 | m-D5 | m-D5 | H | H | H |
| 5537 | 0 | A-38 | m-D5 | m-D5 | H | H | H |
| 5538 | 0 | A-39 | m-D5 | m-D5 | H | H | H |
| 5539 | 0 | A-40 | m-D5 | m-D5 | H | H | H |
| 5540 | 0 | A-41 | m-D5 | m-D5 | H | H | H |
| 5541 | 0 | A-42 | m-D5 | m-D5 | H | H | H |
| 5542 | 0 | A-43 | m-D5 | m-D5 | H | H | H |
| 5543 | 0 | A-44 | m-D5 | m-D5 | H | H | H |
| 5544 | 0 | A-45 | m-D5 | m-D5 | H | H | H |
| 5545 | 0 | A-46 | m-D5 | m-D5 | H | H | H |
| 5546 | 0 | A-47 | m-D5 | m-D5 | H | H | H |
| 5547 | 0 | A-1 | m-D9 | m-D9 | H | H | H |
| 5548 | 0 | A-2 | m-D9 | m-D9 | H | H | H |
| 5549 | 0 | A-3 | m-D9 | m-D9 | H | H | H |
| 5550 | 0 | A-4 | m-D9 | m-D9 | H | H | H |
| 5551 | 0 | A-5 | m-D9 | m-D9 | H | H | H |
| 5552 | 0 | A-6 | m-D9 | m-D9 | H | H | H |
| 5553 | 0 | A-7 | m-D9 | m-D9 | H | H | H |
| 5554 | 0 | A-8 | m-D9 | m-D9 | H | H | H |
| 5555 | 0 | A-9 | m-D9 | m-D9 | H | H | H |
| 5556 | 0 | A-10 | m-D9 | m-D9 | H | H | H |
| 5557 | 0 | A-11 | m-D9 | m-D9 | H | H | H |
| 5558 | 0 | A-12 | m-D9 | m-D9 | H | H | H |
| 5559 | 0 | A-13 | m-D9 | m-D9 | H | H | H |
| 5560 | 0 | A-14 | m-D9 | m-D9 | H | H | H |
| 5561 | 0 | A-15 | m-D9 | m-D9 | H | H | H |
| 5562 | 0 | A-16 | m-D9 | m-D9 | H | H | H |
| 5563 | 0 | A-17 | m-D9 | m-D9 | H | H | H |
| 5564 | 0 | A-18 | m-D9 | m-D9 | H | H | H |
| 5565 | 0 | A-19 | m-D9 | m-D9 | H | H | H |
| 5566 | 0 | A-20 | m-D9 | m-D9 | H | H | H |
| 5567 | 0 | A-21 | m-D9 | m-D9 | H | H | H |
| 5568 | 0 | A-22 | m-D9 | m-D9 | H | H | H |
| 5569 | 0 | A-23 | m-D9 | m-D9 | H | H | H |
| 5570 | 0 | A-24 | m-D9 | m-D9 | H | H | H |
| 5571 | 0 | A-25 | m-D9 | m-D9 | H | H | H |
| 5572 | 0 | A-26 | m-D9 | m-D9 | H | H | H |
| 5573 | 0 | A-27 | m-D9 | m-D9 | H | H | H |
| 5574 | 0 | A-28 | m-D9 | m-D9 | H | H | H |
| 5575 | 0 | A-29 | m-D9 | m-D9 | H | H | H |
| 5576 | 0 | A-30 | m-D9 | m-D9 | H | H | H |
| 5577 | 0 | A-31 | m-D9 | m-D9 | H | H | H |
| 5578 | 0 | A-32 | m-D9 | m-D9 | H | H | H |
| 5579 | 0 | A-33 | m-D9 | m-D9 | H | H | H |
| 5580 | 0 | A-34 | m-D9 | m-D9 | H | H | H |
| 5581 | 0 | A-35 | m-D9 | m-D9 | H | H | H |
| 5582 | 0 | A-36 | m-D9 | m-D9 | H | H | H |
| 5583 | 0 | A-37 | m-D9 | m-D9 | H | H | H |
| 5584 | 0 | A-38 | m-D9 | m-D9 | H | H | H |
| 5585 | 0 | A-39 | m-D9 | m-D9 | H | H | H |
| 5586 | 0 | A-40 | m-D9 | m-D9 | H | H | H |
| 5587 | 0 | A-41 | m-D9 | m-D9 | H | H | H |
| 5588 | 0 | A-42 | m-D9 | m-D9 | H | H | H |
| 5589 | 0 | A-43 | m-D9 | m-D9 | H | H | H |
| 5590 | 0 | A-44 | m-D9 | m-D9 | H | H | H |
| 5591 | 0 | A-45 | m-D9 | m-D9 | H | H | H |
| 5592 | 0 | A-46 | m-D9 | m-D9 | H | H | H |
| 5593 | 0 | A-47 | m-D9 | m-D9 | H | H | H |
| 5594 | 0 | A-1 | m-D11 | m-D11 | H | H | H |
| 5595 | 0 | A-2 | m-D11 | m-D11 | H | H | H |
| 5596 | 0 | A-3 | m-D11 | m-D11 | H | H | H |
| 5597 | 0 | A-4 | m-D11 | m-D11 | H | H | H |
| 5598 | 0 | A-5 | m-D11 | m-D11 | H | H | H |
| 5599 | 0 | A-6 | m-D11 | m-D11 | H | H | H |
| 5600 | 0 | A-7 | m-D11 | m-D11 | H | H | H |
| 5601 | 0 | A-8 | m-D11 | m-D11 | H | H | H |
| 5602 | 0 | A-9 | m-D11 | m-D11 | H | H | H |
| 5603 | 0 | A-10 | m-D11 | m-D11 | H | H | H |
| 5604 | 0 | A-11 | m-D11 | m-D11 | H | H | H |
| 5605 | 0 | A-12 | m-D11 | m-D11 | H | H | H |
| 5606 | 0 | A-13 | m-D11 | m-D11 | H | H | H |
| 5607 | 0 | A-14 | m-D11 | m-D11 | H | H | H |
| 5608 | 0 | A-15 | m-D11 | m-D11 | H | H | H |
| 5609 | 0 | A-16 | m-D11 | m-D11 | H | H | H |
| 5610 | 0 | A-17 | m-D11 | m-D11 | H | H | H |
| 5611 | 0 | A-18 | m-D11 | m-D11 | H | H | H |
| 5612 | 0 | A-19 | m-D11 | m-D11 | H | H | H |
| 5613 | 0 | A-20 | m-D11 | m-D11 | H | H | H |
| 5614 | 0 | A-21 | m-D11 | m-D11 | H | H | H |
| 5615 | 0 | A-22 | m-D11 | m-D11 | H | H | H |
| 5616 | 0 | A-23 | m-D11 | m-D11 | H | H | H |
| 5617 | 0 | A-24 | m-D11 | m-D11 | H | H | H |
| 5618 | 0 | A-25 | m-D11 | m-D11 | H | H | H |
| 5619 | 0 | A-26 | m-D11 | m-D11 | H | H | H |
| 5620 | 0 | A-27 | m-D11 | m-D11 | H | H | H |
| 5621 | 0 | A-28 | m-D11 | m-D11 | H | H | H |
| 5622 | 0 | A-29 | m-D11 | m-D11 | H | H | H |
| 5623 | 0 | A-30 | m-D11 | m-D11 | H | H | H |
| 5624 | 0 | A-31 | m-D11 | m-D11 | H | H | H |
| 5625 | 0 | A-32 | m-D11 | m-D11 | H | H | H |
| 5626 | 0 | A-33 | m-D11 | m-D11 | H | H | H |
| 5627 | 0 | A-34 | m-D11 | m-D11 | H | H | H |
| 5628 | 0 | A-35 | m-D11 | m-D11 | H | H | H |
| 5629 | 0 | A-36 | m-D11 | m-D11 | H | H | H |
| 5630 | 0 | A-37 | m-D11 | m-D11 | H | H | H |
| 5631 | 0 | A-38 | m-D11 | m-D11 | H | H | H |
| 5632 | 0 | A-39 | m-D11 | m-D11 | H | H | H |
| 5633 | 0 | A-40 | m-D11 | m-D11 | H | H | H |
| 5634 | 0 | A-41 | m-D11 | m-D11 | H | H | H |
| 5635 | 0 | A-42 | m-D11 | m-D11 | H | H | H |
| 5636 | 0 | A-43 | m-D11 | m-D11 | H | H | H |
| 5637 | 0 | A-44 | m-D11 | m-D11 | H | H | H |
| 5638 | 0 | A-45 | m-D11 | m-D11 | H | H | H |
| 5639 | 0 | A-46 | m-D11 | m-D11 | H | H | H |
| 5640 | 0 | A-47 | m-D11 | m-D11 | H | H | H |
| 5641 | 0 | A-1 | m-D12 | m-D12 | H | H | H |
| 5642 | 0 | A-2 | m-D12 | m-D12 | H | H | H |
| 5643 | 0 | A-3 | m-D12 | m-D12 | H | H | H |
| 5644 | 0 | A-4 | m-D12 | m-D12 | H | H | H |
| 5645 | 0 | A-5 | m-D12 | m-D12 | H | H | H |
| 5646 | 0 | A-6 | m-D12 | m-D12 | H | H | H |
| 5647 | 0 | A-7 | m-D12 | m-D12 | H | H | H |
| 5648 | 0 | A-8 | m-D12 | m-D12 | H | H | H |
| 5649 | 0 | A-9 | m-D12 | m-D12 | H | H | H |
| 5650 | 0 | A-10 | m-D12 | m-D12 | H | H | H |
| 5651 | 0 | A-11 | m-D12 | m-D12 | H | H | H |
| 5652 | 0 | A-12 | m-D12 | m-D12 | H | H | H |
| 5653 | 0 | A-13 | m-D12 | m-D12 | H | H | H |
| 5654 | 0 | A-14 | m-D12 | m-D12 | H | H | H |
| 5655 | 0 | A-15 | m-D12 | m-D12 | H | H | H |
| 5656 | 0 | A-16 | m-D12 | m-D12 | H | H | H |
| 5657 | 0 | A-17 | m-D12 | m-D12 | H | H | H |
| 5658 | 0 | A-18 | m-D12 | m-D12 | H | H | H |
| 5659 | 0 | A-19 | m-D12 | m-D12 | H | H | H |
| 5660 | 0 | A-20 | m-D12 | m-D12 | H | H | H |
| 5661 | 0 | A-21 | m-D12 | m-D12 | H | H | H |
| 5662 | 0 | A-22 | m-D12 | m-D12 | H | H | H |
| 5663 | 0 | A-23 | m-D12 | m-D12 | H | H | H |
| 5664 | 0 | A-24 | m-D12 | m-D12 | H | H | H |
| 5665 | 0 | A-25 | m-D12 | m-D12 | H | H | H |
| 5666 | 0 | A-26 | m-D12 | m-D12 | H | H | H |
| 5667 | 0 | A-27 | m-D12 | m-D12 | H | H | H |
| 5668 | 0 | A-28 | m-D12 | m-D12 | H | H | H |
| 5669 | 0 | A-29 | m-D12 | m-D12 | H | H | H |
| 5670 | 0 | A-30 | m-D12 | m-D12 | H | H | H |
| 5671 | 0 | A-31 | m-D12 | m-D12 | H | H | H |
| 5672 | 0 | A-32 | m-D12 | m-D12 | H | H | H |
| 5673 | 0 | A-33 | m-D12 | m-D12 | H | H | H |
| 5674 | 0 | A-34 | m-D12 | m-D12 | H | H | H |
| 5675 | 0 | A-35 | m-D12 | m-D12 | H | H | H |
| 5676 | 0 | A-36 | m-D12 | m-D12 | H | H | H |
| 5677 | 0 | A-37 | m-D12 | m-D12 | H | H | H |
| 5678 | 0 | A-38 | m-D12 | m-D12 | H | H | H |
| 5679 | 0 | A-39 | m-D12 | m-D12 | H | H | H |
| 5680 | 0 | A-40 | m-D12 | m-D12 | H | H | H |
| 5681 | 0 | A-41 | m-D12 | m-D12 | H | H | H |
| 5682 | 0 | A-42 | m-D12 | m-D12 | H | H | H |
| 5683 | 0 | A-43 | m-D12 | m-D12 | H | H | H |
| 5684 | 0 | A-44 | m-D12 | m-D12 | H | H | H |
| 5685 | 0 | A-45 | m-D12 | m-D12 | H | H | H |
| 5686 | 0 | A-46 | m-D12 | m-D12 | H | H | H |
| 5687 | 0 | A-47 | m-D12 | m-D12 | H | H | H |
| 5688 | 0 | A-1 | m-D13 | m-D13 | H | H | H |
| 5689 | 0 | A-2 | m-D13 | m-D13 | H | H | H |
| 5690 | 0 | A-3 | m-D13 | m-D13 | H | H | H |
| 5691 | 0 | A-4 | m-D13 | m-D13 | H | H | H |
| 5692 | 0 | A-5 | m-D13 | m-D13 | H | H | H |
| 5693 | 0 | A-6 | m-D13 | m-D13 | H | H | H |
| 5694 | 0 | A-7 | m-D13 | m-D13 | H | H | H |
| 5695 | 0 | A-8 | m-D13 | m-D13 | H | H | H |
| 5696 | 0 | A-9 | m-D13 | m-D13 | H | H | H |
| 5697 | 0 | A-10 | m-D13 | m-D13 | H | H | H |
| 5698 | 0 | A-11 | m-D13 | m-D13 | H | H | H |
| 5699 | 0 | A-12 | m-D13 | m-D13 | H | H | H |
| 5700 | 0 | A-13 | m-D13 | m-D13 | H | H | H |
| 5701 | 0 | A-14 | m-D13 | m-D13 | H | H | H |
| 5702 | 0 | A-15 | m-D13 | m-D13 | H | H | H |
| 5703 | 0 | A-16 | m-D13 | m-D13 | H | H | H |
| 5704 | 0 | A-17 | m-D13 | m-D13 | H | H | H |
| 5705 | 0 | A-18 | m-D13 | m-D13 | H | H | H |
| 5706 | 0 | A-19 | m-D13 | m-D13 | H | H | H |
| 5707 | 0 | A-20 | m-D13 | m-D13 | H | H | H |
| 5708 | 0 | A-21 | m-D13 | m-D13 | H | H | H |
| 5709 | 0 | A-22 | m-D13 | m-D13 | H | H | H |
| 5710 | 0 | A-23 | m-D13 | m-D13 | H | H | H |
| 5711 | 0 | A-24 | m-D13 | m-D13 | H | H | H |
| 5712 | 0 | A-25 | m-D13 | m-D13 | H | H | H |
| 5713 | 0 | A-26 | m-D13 | m-D13 | H | H | H |
| 5714 | 0 | A-27 | m-D13 | m-D13 | H | H | H |
| 5715 | 0 | A-28 | m-D13 | m-D13 | H | H | H |
| 5716 | 0 | A-29 | m-D13 | m-D13 | H | H | H |
| 5717 | 0 | A-30 | m-D13 | m-D13 | H | H | H |
| 5718 | 0 | A-31 | m-D13 | m-D13 | H | H | H |
| 5719 | 0 | A-32 | m-D13 | m-D13 | H | H | H |
| 5720 | 0 | A-33 | m-D13 | m-D13 | H | H | H |
| 5721 | 0 | A-34 | m-D13 | m-D13 | H | H | H |
| 5722 | 0 | A-35 | m-D13 | m-D13 | H | H | H |
| 5723 | 0 | A-36 | m-D13 | m-D13 | H | H | H |
| 5724 | 0 | A-37 | m-D13 | m-D13 | H | H | H |
| 5725 | 0 | A-38 | m-D13 | m-D13 | H | H | H |
| 5726 | 0 | A-39 | m-D13 | m-D13 | H | H | H |
| 5727 | 0 | A-40 | m-D13 | m-D13 | H | H | H |
| 5728 | 0 | A-41 | m-D13 | m-D13 | H | H | H |
| 5729 | 0 | A-42 | m-D13 | m-D13 | H | H | H |
| 5730 | 0 | A-43 | m-D13 | m-D13 | H | H | H |
| 5731 | 0 | A-44 | m-D13 | m-D13 | H | H | H |
| 5732 | 0 | A-45 | m-D13 | m-D13 | H | H | H |
| 5733 | 0 | A-46 | m-D13 | m-D13 | H | H | H |
| 5734 | 0 | A-47 | m-D13 | m-D13 | H | H | H |
| 5735 | 0 | A-1 | m-D14 | m-D14 | H | H | H |
| 5736 | 0 | A-2 | m-D14 | m-D14 | H | H | H |
| 5737 | 0 | A-3 | m-D14 | m-D14 | H | H | H |
| 5738 | 0 | A-4 | m-D14 | m-D14 | H | H | H |
| 5739 | 0 | A-5 | m-D14 | m-D14 | H | H | H |
| 5740 | 0 | A-6 | m-D14 | m-D14 | H | H | H |
| 5741 | 0 | A-7 | m-D14 | m-D14 | H | H | H |
| 5742 | 0 | A-8 | m-D14 | m-D14 | H | H | H |
| 5743 | 0 | A-9 | m-D14 | m-D14 | H | H | H |
| 5744 | 0 | A-10 | m-D14 | m-D14 | H | H | H |
| 5745 | 0 | A-11 | m-D14 | m-D14 | H | H | H |
| 5746 | 0 | A-12 | m-D14 | m-D14 | H | H | H |
| 5747 | 0 | A-13 | m-D14 | m-D14 | H | H | H |
| 5748 | 0 | A-14 | m-D14 | m-D14 | H | H | H |
| 5749 | 0 | A-15 | m-D14 | m-D14 | H | H | H |
| 5750 | 0 | A-16 | m-D14 | m-D14 | H | H | H |
| 5751 | 0 | A-17 | m-D14 | m-D14 | H | H | H |
| 5752 | 0 | A-18 | m-D14 | m-D14 | H | H | H |
| 5753 | 0 | A-19 | m-D14 | m-D14 | H | H | H |
| 5754 | 0 | A-20 | m-D14 | m-D14 | H | H | H |
| 5755 | 0 | A-21 | m-D14 | m-D14 | H | H | H |
| 5756 | 0 | A-22 | m-D14 | m-D14 | H | H | H |
| 5757 | 0 | A-23 | m-D14 | m-D14 | H | H | H |
| 5758 | 0 | A-24 | m-D14 | m-D14 | H | H | H |
| 5759 | 0 | A-25 | m-D14 | m-D14 | H | H | H |
| 5760 | 0 | A-26 | m-D14 | m-D14 | H | H | H |
| 5761 | 0 | A-27 | m-D14 | m-D14 | H | H | H |
| 5762 | 0 | A-28 | m-D14 | m-D14 | H | H | H |
| 5763 | 0 | A-29 | m-D14 | m-D14 | H | H | H |
| 5764 | 0 | A-30 | m-D14 | m-D14 | H | H | H |
| 5765 | 0 | A-31 | m-D14 | m-D14 | H | H | H |
| 5766 | 0 | A-32 | m-D14 | m-D14 | H | H | H |
| 5767 | 0 | A-33 | m-D14 | m-D14 | H | H | H |
| 5768 | 0 | A-34 | m-D14 | m-D14 | H | H | H |
| 5769 | 0 | A-35 | m-D14 | m-D14 | H | H | H |
| 5770 | 0 | A-36 | m-D14 | m-D14 | H | H | H |
| 5771 | 0 | A-37 | m-D14 | m-D14 | H | H | H |
| 5772 | 0 | A-38 | m-D14 | m-D14 | H | H | H |
| 5773 | 0 | A-39 | m-D14 | m-D14 | H | H | H |
| 5774 | 0 | A-40 | m-D14 | m-D14 | H | H | H |
| 5775 | 0 | A-41 | m-D14 | m-D14 | H | H | H |
| 5776 | 0 | A-42 | m-D14 | m-D14 | H | H | H |
| 5777 | 0 | A-43 | m-D14 | m-D14 | H | H | H |
| 5778 | 0 | A-44 | m-D14 | m-D14 | H | H | H |
| 5779 | 0 | A-45 | m-D14 | m-D14 | H | H | H |
| 5780 | 0 | A-46 | m-D14 | m-D14 | H | H | H |
| 5781 | 0 | A-47 | m-D14 | m-D14 | H | H | H |
| 5782 | 0 | A-1 | m-D1 | A-1 | H | H | H |
| 5783 | 0 | A-2 | m-D1 | A-2 | H | H | H |
| 5784 | 0 | A-3 | m-D1 | A-3 | H | H | H |
| 5785 | 0 | A-4 | m-D1 | A-4 | H | H | H |
| 5786 | 0 | A-5 | m-D1 | A-5 | H | H | H |
| 5787 | 0 | A-6 | m-D1 | A-6 | H | H | H |
| 5788 | 0 | A-7 | m-D1 | A-7 | H | H | H |
| 5789 | 0 | A-8 | m-D1 | A-8 | H | H | H |
| 5790 | 0 | A-9 | m-D1 | A-9 | H | H | H |
| 5791 | 0 | A-10 | m-D1 | A-10 | H | H | H |
| 5792 | 0 | A-11 | m-D1 | A-11 | H | H | H |
| 5793 | 0 | A-12 | m-D1 | A-12 | H | H | H |
| 5794 | 0 | A-13 | m-D1 | A-13 | H | H | H |
| 5795 | 0 | A-14 | m-D1 | A-14 | H | H | H |
| 5796 | 0 | A-15 | m-D1 | A-15 | H | H | H |
| 5797 | 0 | A-16 | m-D1 | A-16 | H | H | H |
| 5798 | 0 | A-17 | m-D1 | A-17 | H | H | H |
| 5799 | 0 | A-18 | m-D1 | A-18 | H | H | H |
| 5800 | 0 | A-19 | m-D1 | A-19 | H | H | H |
| 5801 | 0 | A-20 | m-D1 | A-20 | H | H | H |
| 5802 | 0 | A-21 | m-D1 | A-21 | H | H | H |
| 5803 | 0 | A-22 | m-D1 | A-22 | H | H | H |
| 5804 | 0 | A-23 | m-D1 | A-23 | H | H | H |
| 5805 | 0 | A-24 | m-D1 | A-24 | H | H | H |
| 5806 | 0 | A-25 | m-D1 | A-25 | H | H | H |
| 5807 | 0 | A-26 | m-D1 | A-26 | H | H | H |
| 5808 | 0 | A-27 | m-D1 | A-27 | H | H | H |
| 5809 | 0 | A-28 | m-D1 | A-28 | H | H | H |
| 5810 | 0 | A-29 | m-D1 | A-29 | H | H | H |
| 5811 | 0 | A-30 | m-D1 | A-30 | H | H | H |
| 5812 | 0 | A-31 | m-D1 | A-31 | H | H | H |
| 5813 | 0 | A-32 | m-D1 | A-32 | H | H | H |
| 5814 | 0 | A-33 | m-D1 | A-33 | H | H | H |
| 5815 | 0 | A-34 | m-D1 | A-34 | H | H | H |
| 5816 | 0 | A-35 | m-D1 | A-35 | H | H | H |
| 5817 | 0 | A-36 | m-D1 | A-36 | H | H | H |
| 5818 | 0 | A-37 | m-D1 | A-37 | H | H | H |
| 5819 | 0 | A-38 | m-D1 | A-38 | H | H | H |
| 5820 | 0 | A-39 | m-D1 | A-39 | H | H | H |
| 5821 | 0 | A-40 | m-D1 | A-40 | H | H | H |
| 5822 | 0 | A-41 | m-D1 | A-41 | H | H | H |
| 5823 | 0 | A-42 | m-D1 | A-42 | H | H | H |
| 5824 | 0 | A-43 | m-D1 | A-43 | H | H | H |
| 5825 | 0 | A-44 | m-D1 | A-44 | H | H | H |
| 5826 | 0 | A-45 | m-D1 | A-45 | H | H | H |
| 5827 | 0 | A-46 | m-D1 | A-46 | H | H | H |
| 5828 | 0 | A-47 | m-D1 | A-47 | H | H | H |
| 5829 | 0 | A-1 | m-D2 | A-1 | H | H | H |
| 5830 | 0 | A-2 | m-D2 | A-2 | H | H | H |
| 5831 | 0 | A-3 | m-D2 | A-3 | H | H | H |
| 5832 | 0 | A-4 | m-D2 | A-4 | H | H | H |
| 5833 | 0 | A-5 | m-D2 | A-5 | H | H | H |
| 5834 | 0 | A-6 | m-D2 | A-6 | H | H | H |
| 5835 | 0 | A-7 | m-D2 | A-7 | H | H | H |
| 5836 | 0 | A-8 | m-D2 | A-8 | H | H | H |
| 5837 | 0 | A-9 | m-D2 | A-9 | H | H | H |
| 5838 | 0 | A-10 | m-D2 | A-10 | H | H | H |
| 5839 | 0 | A-11 | m-D2 | A-11 | H | H | H |
| 5840 | 0 | A-12 | m-D2 | A-12 | H | H | H |
| 5841 | 0 | A-13 | m-D2 | A-13 | H | H | H |
| 5842 | 0 | A-14 | m-D2 | A-14 | H | H | H |
| 5843 | 0 | A-15 | m-D2 | A-15 | H | H | H |
| 5844 | 0 | A-16 | m-D2 | A-16 | H | H | H |
| 5845 | 0 | A-17 | m-D2 | A-17 | H | H | H |
| 5846 | 0 | A-18 | m-D2 | A-18 | H | H | H |
| 5847 | 0 | A-19 | m-D2 | A-19 | H | H | H |
| 5848 | 0 | A-20 | m-D2 | A-20 | H | H | H |
| 5849 | 0 | A-21 | m-D2 | A-21 | H | H | H |
| 5850 | 0 | A-22 | m-D2 | A-22 | H | H | H |
| 5851 | 0 | A-23 | m-D2 | A-23 | H | H | H |
| 5852 | 0 | A-24 | m-D2 | A-24 | H | H | H |
| 5853 | 0 | A-25 | m-D2 | A-25 | H | H | H |
| 5854 | 0 | A-26 | m-D2 | A-26 | H | H | H |
| 5855 | 0 | A-27 | m-D2 | A-27 | H | H | H |
| 5856 | 0 | A-28 | m-D2 | A-28 | H | H | H |
| 5857 | 0 | A-29 | m-D2 | A-29 | H | H | H |
| 5858 | 0 | A-30 | m-D2 | A-30 | H | H | H |
| 5859 | 0 | A-31 | m-D2 | A-31 | H | H | H |
| 5860 | 0 | A-32 | m-D2 | A-32 | H | H | H |
| 5861 | 0 | A-33 | m-D2 | A-33 | H | H | H |
| 5862 | 0 | A-34 | m-D2 | A-34 | H | H | H |
| 5863 | 0 | A-35 | m-D2 | A-35 | H | H | H |
| 5864 | 0 | A-36 | m-D2 | A-36 | H | H | H |
| 5865 | 0 | A-37 | m-D2 | A-37 | H | H | H |
| 5866 | 0 | A-38 | m-D2 | A-38 | H | H | H |
| 5867 | 0 | A-39 | m-D2 | A-39 | H | H | H |
| 5868 | 0 | A-40 | m-D2 | A-40 | H | H | H |
| 5869 | 0 | A-41 | m-D2 | A-41 | H | H | H |
| 5870 | 0 | A-42 | m-D2 | A-42 | H | H | H |
| 5871 | 0 | A-43 | m-D2 | A-43 | H | H | H |
| 5872 | 0 | A-44 | m-D2 | A-44 | H | H | H |
| 5873 | 0 | A-45 | m-D2 | A-45 | H | H | H |
| 5874 | 0 | A-46 | m-D2 | A-46 | H | H | H |
| 5875 | 0 | A-47 | m-D2 | A-47 | H | H | H |
| 5876 | 0 | A-1 | m-D3 | A-1 | H | H | H |
| 5877 | 0 | A-2 | m-D3 | A-2 | H | H | H |
| 5878 | 0 | A-3 | m-D3 | A-3 | H | H | H |
| 5879 | 0 | A-4 | m-D3 | A-4 | H | H | H |
| 5880 | 0 | A-5 | m-D3 | A-5 | H | H | H |
| 5881 | 0 | A-6 | m-D3 | A-6 | H | H | H |
| 5882 | 0 | A-7 | m-D3 | A-7 | H | H | H |
| 5883 | 0 | A-8 | m-D3 | A-8 | H | H | H |
| 5884 | 0 | A-9 | m-D3 | A-9 | H | H | H |
| 5885 | 0 | A-10 | m-D3 | A-10 | H | H | H |
| 5886 | 0 | A-11 | m-D3 | A-11 | H | H | H |
| 5887 | 0 | A-12 | m-D3 | A-12 | H | H | H |
| 5888 | 0 | A-13 | m-D3 | A-13 | H | H | H |
| 5889 | 0 | A-14 | m-D3 | A-14 | H | H | H |
| 5890 | 0 | A-15 | m-D3 | A-15 | H | H | H |
| 5891 | 0 | A-16 | m-D3 | A-16 | H | H | H |
| 5892 | 0 | A-17 | m-D3 | A-17 | H | H | H |
| 5893 | 0 | A-18 | m-D3 | A-18 | H | H | H |
| 5894 | 0 | A-19 | m-D3 | A-19 | H | H | H |
| 5895 | 0 | A-20 | m-D3 | A-20 | H | H | H |
| 5896 | 0 | A-21 | m-D3 | A-21 | H | H | H |
| 5897 | 0 | A-22 | m-D3 | A-22 | H | H | H |
| 5898 | 0 | A-23 | m-D3 | A-23 | H | H | H |
| 5899 | 0 | A-24 | m-D3 | A-24 | H | H | H |
| 5900 | 0 | A-25 | m-D3 | A-25 | H | H | H |
| 5901 | 0 | A-26 | m-D3 | A-26 | H | H | H |
| 5902 | 0 | A-27 | m-D3 | A-27 | H | H | H |
| 5903 | 0 | A-28 | m-D3 | A-28 | H | H | H |
| 5904 | 0 | A-29 | m-D3 | A-29 | H | H | H |
| 5905 | 0 | A-30 | m-D3 | A-30 | H | H | H |
| 5906 | 0 | A-31 | m-D3 | A-31 | H | H | H |
| 5907 | 0 | A-32 | m-D3 | A-32 | H | H | H |
| 5908 | 0 | A-33 | m-D3 | A-33 | H | H | H |
| 5909 | 0 | A-34 | m-D3 | A-34 | H | H | H |
| 5910 | 0 | A-35 | m-D3 | A-35 | H | H | H |
| 5911 | 0 | A-36 | m-D3 | A-36 | H | H | H |
| 5912 | 0 | A-37 | m-D3 | A-37 | H | H | H |
| 5913 | 0 | A-38 | m-D3 | A-38 | H | H | H |
| 5914 | 0 | A-39 | m-D3 | A-39 | H | H | H |
| 5915 | 0 | A-40 | m-D3 | A-40 | H | H | H |
| 5916 | 0 | A-41 | m-D3 | A-41 | H | H | H |
| 5917 | 0 | A-42 | m-D3 | A-42 | H | H | H |
| 5918 | 0 | A-43 | m-D3 | A-43 | H | H | H |
| 5919 | 0 | A-44 | m-D3 | A-44 | H | H | H |
| 5920 | 0 | A-45 | m-D3 | A-45 | H | H | H |
| 5921 | 0 | A-46 | m-D3 | A-46 | H | H | H |
| 5922 | 0 | A-47 | m-D3 | A-47 | H | H | H |
| 5923 | 0 | A-1 | m-D5 | A-1 | H | H | H |
| 5924 | 0 | A-2 | m-D5 | A-2 | H | H | H |
| 5925 | 0 | A-3 | m-D5 | A-3 | H | H | H |
| 5926 | 0 | A-4 | m-D5 | A-4 | H | H | H |
| 5927 | 0 | A-5 | m-D5 | A-5 | H | H | H |
| 5928 | 0 | A-6 | m-D5 | A-6 | H | H | H |
| 5929 | 0 | A-7 | m-D5 | A-7 | H | H | H |
| 5930 | 0 | A-8 | m-D5 | A-8 | H | H | H |
| 5931 | 0 | A-9 | m-D5 | A-9 | H | H | H |
| 5932 | 0 | A-10 | m-D5 | A-10 | H | H | H |
| 5933 | 0 | A-11 | m-D5 | A-11 | H | H | H |
| 5934 | 0 | A-12 | m-D5 | A-12 | H | H | H |
| 5935 | 0 | A-13 | m-D5 | A-13 | H | H | H |
| 5936 | 0 | A-14 | m-D5 | A-14 | H | H | H |
| 5937 | 0 | A-15 | m-D5 | A-15 | H | H | H |
| 5938 | 0 | A-16 | m-D5 | A-16 | H | H | H |
| 5939 | 0 | A-17 | m-D5 | A-17 | H | H | H |
| 5940 | 0 | A-18 | m-D5 | A-18 | H | H | H |
| 5941 | 0 | A-19 | m-D5 | A-19 | H | H | H |
| 5942 | 0 | A-20 | m-D5 | A-20 | H | H | H |
| 5943 | 0 | A-21 | m-D5 | A-21 | H | H | H |
| 5944 | 0 | A-22 | m-D5 | A-22 | H | H | H |
| 5945 | 0 | A-23 | m-D5 | A-23 | H | H | H |
| 5946 | 0 | A-24 | m-D5 | A-24 | H | H | H |
| 5947 | 0 | A-25 | m-D5 | A-25 | H | H | H |
| 5948 | 0 | A-26 | m-D5 | A-26 | H | H | H |
| 5949 | 0 | A-27 | m-D5 | A-27 | H | H | H |
| 5950 | 0 | A-28 | m-D5 | A-28 | H | H | H |
| 5951 | 0 | A-29 | m-D5 | A-29 | H | H | H |
| 5952 | 0 | A-30 | m-D5 | A-30 | H | H | H |
| 5953 | 0 | A-31 | m-D5 | A-31 | H | H | H |
| 5954 | 0 | A-32 | m-D5 | A-32 | H | H | H |
| 5955 | 0 | A-33 | m-D5 | A-33 | H | H | H |
| 5956 | 0 | A-34 | m-D5 | A-34 | H | H | H |
| 5957 | 0 | A-35 | m-D5 | A-35 | H | H | H |
| 5958 | 0 | A-36 | m-D5 | A-36 | H | H | H |
| 5959 | 0 | A-37 | m-D5 | A-37 | H | H | H |
| 5960 | 0 | A-38 | m-D5 | A-38 | H | H | H |
| 5961 | 0 | A-39 | m-D5 | A-39 | H | H | H |
| 5962 | 0 | A-40 | m-D5 | A-40 | H | H | H |
| 5963 | 0 | A-41 | m-D5 | A-41 | H | H | H |
| 5964 | 0 | A-42 | m-D5 | A-42 | H | H | H |
| 5965 | 0 | A-43 | m-D5 | A-43 | H | H | H |
| 5966 | 0 | A-44 | m-D5 | A-44 | H | H | H |
| 5967 | 0 | A-45 | m-D5 | A-45 | H | H | H |
| 5968 | 0 | A-46 | m-D5 | A-46 | H | H | H |
| 5969 | 0 | A-47 | m-D5 | A-47 | H | H | H |
| 5970 | 0 | A-1 | m-D9 | A-1 | H | H | H |
| 5971 | 0 | A-2 | m-D9 | A-2 | H | H | H |
| 5972 | 0 | A-3 | m-D9 | A-3 | H | H | H |
| 5973 | 0 | A-4 | m-D9 | A-4 | H | H | H |
| 5974 | 0 | A-5 | m-D9 | A-5 | H | H | H |
| 5975 | 0 | A-6 | m-D9 | A-6 | H | H | H |
| 5976 | 0 | A-7 | m-D9 | A-7 | H | H | H |
| 5977 | 0 | A-8 | m-D9 | A-8 | H | H | H |
| 5978 | 0 | A-9 | m-D9 | A-9 | H | H | H |
| 5979 | 0 | A-10 | m-D9 | A-10 | H | H | H |
| 5980 | 0 | A-11 | m-D9 | A-11 | H | H | H |
| 5981 | 0 | A-12 | m-D9 | A-12 | H | H | H |
| 5982 | 0 | A-13 | m-D9 | A-13 | H | H | H |
| 5983 | 0 | A-14 | m-D9 | A-14 | H | H | H |
| 5984 | 0 | A-15 | m-D9 | A-15 | H | H | H |
| 5985 | 0 | A-16 | m-D9 | A-16 | H | H | H |
| 5986 | 0 | A-17 | m-D9 | A-17 | H | H | H |
| 5987 | 0 | A-18 | m-D9 | A-18 | H | H | H |
| 5988 | 0 | A-19 | m-D9 | A-19 | H | H | H |
| 5989 | 0 | A-20 | m-D9 | A-20 | H | H | H |
| 5990 | 0 | A-21 | m-D9 | A-21 | H | H | H |
| 5991 | 0 | A-22 | m-D9 | A-22 | H | H | H |
| 5992 | 0 | A-23 | m-D9 | A-23 | H | H | H |
| 5993 | 0 | A-24 | m-D9 | A-24 | H | H | H |
| 5994 | 0 | A-25 | m-D9 | A-25 | H | H | H |
| 5995 | 0 | A-26 | m-D9 | A-26 | H | H | H |
| 5996 | 0 | A-27 | m-D9 | A-27 | H | H | H |
| 5997 | 0 | A-28 | m-D9 | A-28 | H | H | H |
| 5998 | 0 | A-29 | m-D9 | A-29 | H | H | H |
| 5999 | 0 | A-30 | m-D9 | A-30 | H | H | H |
| 6000 | 0 | A-31 | m-D9 | A-31 | H | H | H |
| 6001 | 0 | A-32 | m-D9 | A-32 | H | H | H |
| 6002 | 0 | A-33 | m-D9 | A-33 | H | H | H |
| 6003 | 0 | A-34 | m-D9 | A-34 | H | H | H |
| 6004 | 0 | A-35 | m-D9 | A-35 | H | H | H |
| 6005 | 0 | A-36 | m-D9 | A-36 | H | H | H |
| 6006 | 0 | A-37 | m-D9 | A-37 | H | H | H |
| 6007 | 0 | A-38 | m-D9 | A-38 | H | H | H |
| 6008 | 0 | A-39 | m-D9 | A-39 | H | H | H |
| 6009 | 0 | A-40 | m-D9 | A-40 | H | H | H |
| 6010 | 0 | A-41 | m-D9 | A-41 | H | H | H |
| 6011 | 0 | A-42 | m-D9 | A-42 | H | H | H |
| 6012 | 0 | A-43 | m-D9 | A-43 | H | H | H |
| 6013 | 0 | A-44 | m-D9 | A-44 | H | H | H |
| 6014 | 0 | A-45 | m-D9 | A-45 | H | H | H |
| 6015 | 0 | A-46 | m-D9 | A-46 | H | H | H |
| 6016 | 0 | A-47 | m-D9 | A-47 | H | H | H |
| 6017 | 0 | A-1 | m-D11 | A-1 | H | H | H |
| 6018 | 0 | A-2 | m-D11 | A-2 | H | H | H |
| 6019 | 0 | A-3 | m-D11 | A-3 | H | H | H |
| 6020 | 0 | A-4 | m-D11 | A-4 | H | H | H |
| 6021 | 0 | A-5 | m-D11 | A-5 | H | H | H |
| 6022 | 0 | A-6 | m-D11 | A-6 | H | H | H |
| 6023 | 0 | A-7 | m-D11 | A-7 | H | H | H |
| 6024 | 0 | A-8 | m-D11 | A-8 | H | H | H |
| 6025 | 0 | A-9 | m-D11 | A-9 | H | H | H |
| 6026 | 0 | A-10 | m-D11 | A-10 | H | H | H |
| 6027 | 0 | A-11 | m-D11 | A-11 | H | H | H |
| 6028 | 0 | A-12 | m-D11 | A-12 | H | H | H |
| 6029 | 0 | A-13 | m-D11 | A-13 | H | H | H |
| 6030 | 0 | A-14 | m-D11 | A-14 | H | H | H |
| 6031 | 0 | A-15 | m-D11 | A-15 | H | H | H |
| 6032 | 0 | A-16 | m-D11 | A-16 | H | H | H |
| 6033 | 0 | A-17 | m-D11 | A-17 | H | H | H |
| 6034 | 0 | A-18 | m-D11 | A-18 | H | H | H |
| 6035 | 0 | A-19 | m-D11 | A-19 | H | H | H |
| 6036 | 0 | A-20 | m-D11 | A-20 | H | H | H |
| 6037 | 0 | A-21 | m-D11 | A-21 | H | H | H |
| 6038 | 0 | A-22 | m-D11 | A-22 | H | H | H |
| 6039 | 0 | A-23 | m-D11 | A-23 | H | H | H |
| 6040 | 0 | A-24 | m-D11 | A-24 | H | H | H |
| 6041 | 0 | A-25 | m-D11 | A-25 | H | H | H |
| 6042 | 0 | A-26 | m-D11 | A-26 | H | H | H |
| 6043 | 0 | A-27 | m-D11 | A-27 | H | H | H |
| 6044 | 0 | A-28 | m-D11 | A-28 | H | H | H |
| 6045 | 0 | A-29 | m-D11 | A-29 | H | H | H |
| 6046 | 0 | A-30 | m-D11 | A-30 | H | H | H |
| 6047 | 0 | A-31 | m-D11 | A-31 | H | H | H |
| 6048 | 0 | A-32 | m-D11 | A-32 | H | H | H |
| 6049 | 0 | A-33 | m-D11 | A-33 | H | H | H |
| 6050 | 0 | A-34 | m-D11 | A-34 | H | H | H |
| 6051 | 0 | A-35 | m-D11 | A-35 | H | H | H |
| 6052 | 0 | A-36 | m-D11 | A-36 | H | H | H |
| 6053 | 0 | A-37 | m-D11 | A-37 | H | H | H |
| 6054 | 0 | A-38 | m-D11 | A-38 | H | H | H |
| 6055 | 0 | A-39 | m-D11 | A-39 | H | H | H |
| 6056 | 0 | A-40 | m-D11 | A-40 | H | H | H |
| 6057 | 0 | A-41 | m-D11 | A-41 | H | H | H |
| 6058 | 0 | A-42 | m-D11 | A-42 | H | H | H |
| 6059 | 0 | A-43 | m-D11 | A-43 | H | H | H |
| 6060 | 0 | A-44 | m-D11 | A-44 | H | H | H |
| 6061 | 0 | A-45 | m-D11 | A-45 | H | H | H |
| 6062 | 0 | A-46 | m-D11 | A-46 | H | H | H |
| 6063 | 0 | A-47 | m-D11 | A-47 | H | H | H |
| 6064 | 0 | A-1 | m-D12 | A-1 | H | H | H |
| 6065 | 0 | A-2 | m-D12 | A-2 | H | H | H |
| 6066 | 0 | A-3 | m-D12 | A-3 | H | H | H |
| 6067 | 0 | A-4 | m-D12 | A-4 | H | H | H |
| 6068 | 0 | A-5 | m-D12 | A-5 | H | H | H |
| 6069 | 0 | A-6 | m-D12 | A-6 | H | H | H |
| 6070 | 0 | A-7 | m-D12 | A-7 | H | H | H |
| 6071 | 0 | A-8 | m-D12 | A-8 | H | H | H |
| 6072 | 0 | A-9 | m-D12 | A-9 | H | H | H |
| 6073 | 0 | A-10 | m-D12 | A-10 | H | H | H |
| 6074 | 0 | A-11 | m-D12 | A-11 | H | H | H |
| 6075 | 0 | A-12 | m-D12 | A-12 | H | H | H |
| 6076 | 0 | A-13 | m-D12 | A-13 | H | H | H |
| 6077 | 0 | A-14 | m-D12 | A-14 | H | H | H |
| 6078 | 0 | A-15 | m-D12 | A-15 | H | H | H |
| 6079 | 0 | A-16 | m-D12 | A-16 | H | H | H |
| 6080 | 0 | A-17 | m-D12 | A-17 | H | H | H |
| 6081 | 0 | A-18 | m-D12 | A-18 | H | H | H |
| 6082 | 0 | A-19 | m-D12 | A-19 | H | H | H |
| 6083 | 0 | A-20 | m-D12 | A-20 | H | H | H |
| 6084 | 0 | A-21 | m-D12 | A-21 | H | H | H |
| 6085 | 0 | A-22 | m-D12 | A-22 | H | H | H |
| 6086 | 0 | A-23 | m-D12 | A-23 | H | H | H |
| 6087 | 0 | A-24 | m-D12 | A-24 | H | H | H |
| 6088 | 0 | A-25 | m-D12 | A-25 | H | H | H |
| 6089 | 0 | A-26 | m-D12 | A-26 | H | H | H |
| 6090 | 0 | A-27 | m-D12 | A-27 | H | H | H |
| 6091 | 0 | A-28 | m-D12 | A-28 | H | H | H |
| 6092 | 0 | A-29 | m-D12 | A-29 | H | H | H |
| 6093 | 0 | A-30 | m-D12 | A-30 | H | H | H |
| 6094 | 0 | A-31 | m-D12 | A-31 | H | H | H |
| 6095 | 0 | A-32 | m-D12 | A-32 | H | H | H |
| 6096 | 0 | A-33 | m-D12 | A-33 | H | H | H |
| 6097 | 0 | A-34 | m-D12 | A-34 | H | H | H |
| 6098 | 0 | A-35 | m-D12 | A-35 | H | H | H |
| 6099 | 0 | A-36 | m-D12 | A-36 | H | H | H |
| 6100 | 0 | A-37 | m-D12 | A-37 | H | H | H |
| 6101 | 0 | A-38 | m-D12 | A-38 | H | H | H |
| 6102 | 0 | A-39 | m-D12 | A-39 | H | H | H |
| 6103 | 0 | A-40 | m-D12 | A-40 | H | H | H |
| 6104 | 0 | A-41 | m-D12 | A-41 | H | H | H |
| 6105 | 0 | A-42 | m-D12 | A-42 | H | H | H |
| 6106 | 0 | A-43 | m-D12 | A-43 | H | H | H |
| 6107 | 0 | A-44 | m-D12 | A-44 | H | H | H |
| 6108 | 0 | A-45 | m-D12 | A-45 | H | H | H |
| 6109 | 0 | A-46 | m-D12 | A-46 | H | H | H |
| 6110 | 0 | A-47 | m-D12 | A-47 | H | H | H |
| 6111 | 0 | A-1 | m-D13 | A-1 | H | H | H |
| 6112 | 0 | A-2 | m-D13 | A-2 | H | H | H |
| 6113 | 0 | A-3 | m-D13 | A-3 | H | H | H |
| 6114 | 0 | A-4 | m-D13 | A-4 | H | H | H |
| 6115 | 0 | A-5 | m-D13 | A-5 | H | H | H |
| 6116 | 0 | A-6 | m-D13 | A-6 | H | H | H |
| 6117 | 0 | A-7 | m-D13 | A-7 | H | H | H |
| 6118 | 0 | A-8 | m-D13 | A-8 | H | H | H |
| 6119 | 0 | A-9 | m-D13 | A-9 | H | H | H |
| 6120 | 0 | A-10 | m-D13 | A-10 | H | H | H |
| 6121 | 0 | A-11 | m-D13 | A-11 | H | H | H |
| 6122 | 0 | A-12 | m-D13 | A-12 | H | H | H |
| 6123 | 0 | A-13 | m-D13 | A-13 | H | H | H |
| 6124 | 0 | A-14 | m-D13 | A-14 | H | H | H |
| 6125 | 0 | A-15 | m-D13 | A-15 | H | H | H |
| 6126 | 0 | A-16 | m-D13 | A-16 | H | H | H |
| 6127 | 0 | A-17 | m-D13 | A-17 | H | H | H |
| 6128 | 0 | A-18 | m-D13 | A-18 | H | H | H |
| 6129 | 0 | A-19 | m-D13 | A-19 | H | H | H |
| 6130 | 0 | A-20 | m-D13 | A-20 | H | H | H |
| 6131 | 0 | A-21 | m-D13 | A-21 | H | H | H |
| 6132 | 0 | A-22 | m-D13 | A-22 | H | H | H |
| 6133 | 0 | A-23 | m-D13 | A-23 | H | H | H |
| 6134 | 0 | A-24 | m-D13 | A-24 | H | H | H |
| 6135 | 0 | A-25 | m-D13 | A-25 | H | H | H |
| 6136 | 0 | A-26 | m-D13 | A-26 | H | H | H |
| 6137 | 0 | A-27 | m-D13 | A-27 | H | H | H |
| 6138 | 0 | A-28 | m-D13 | A-28 | H | H | H |
| 6139 | 0 | A-29 | m-D13 | A-29 | H | H | H |
| 6140 | 0 | A-30 | m-D13 | A-30 | H | H | H |
| 6141 | 0 | A-31 | m-D13 | A-31 | H | H | H |
| 6142 | 0 | A-32 | m-D13 | A-32 | H | H | H |
| 6143 | 0 | A-33 | m-D13 | A-33 | H | H | H |
| 6144 | 0 | A-34 | m-D13 | A-34 | H | H | H |
| 6145 | 0 | A-35 | m-D13 | A-35 | H | H | H |
| 6146 | 0 | A-36 | m-D13 | A-36 | H | H | H |
| 6147 | 0 | A-37 | m-D13 | A-37 | H | H | H |
| 6148 | 0 | A-38 | m-D13 | A-38 | H | H | H |
| 6149 | 0 | A-39 | m-D13 | A-39 | H | H | H |
| 6150 | 0 | A-40 | m-D13 | A-40 | H | H | H |
| 6151 | 0 | A-41 | m-D13 | A-41 | H | H | H |
| 6152 | 0 | A-42 | m-D13 | A-42 | H | H | H |
| 6153 | 0 | A-43 | m-D13 | A-43 | H | H | H |
| 6154 | 0 | A-44 | m-D13 | A-44 | H | H | H |
| 6155 | 0 | A-45 | m-D13 | A-45 | H | H | H |
| 6156 | 0 | A-46 | m-D13 | A-46 | H | H | H |
| 6157 | 0 | A-47 | m-D13 | A-47 | H | H | H |
| 6158 | 0 | A-1 | m-D14 | A-1 | H | H | H |
| 6159 | 0 | A-2 | m-D14 | A-2 | H | H | H |
| 6160 | 0 | A-3 | m-D14 | A-3 | H | H | H |
| 6161 | 0 | A-4 | m-D14 | A-4 | H | H | H |
| 6162 | 0 | A-5 | m-D14 | A-5 | H | H | H |
| 6163 | 0 | A-6 | m-D14 | A-6 | H | H | H |
| 6164 | 0 | A-7 | m-D14 | A-7 | H | H | H |
| 6165 | 0 | A-8 | m-D14 | A-8 | H | H | H |
| 6166 | 0 | A-9 | m-D14 | A-9 | H | H | H |
| 6167 | 0 | A-10 | m-D14 | A-10 | H | H | H |
| 6168 | 0 | A-11 | m-D14 | A-11 | H | H | H |
| 6169 | 0 | A-12 | m-D14 | A-12 | H | H | H |
| 6170 | 0 | A-13 | m-D14 | A-13 | H | H | H |
| 6171 | 0 | A-14 | m-D14 | A-14 | H | H | H |
| 6172 | 0 | A-15 | m-D14 | A-15 | H | H | H |
| 6173 | 0 | A-16 | m-D14 | A-16 | H | H | H |
| 6174 | 0 | A-17 | m-D14 | A-17 | H | H | H |
| 6175 | 0 | A-18 | m-D14 | A-18 | H | H | H |
| 6176 | 0 | A-19 | m-D14 | A-19 | H | H | H |
| 6177 | 0 | A-20 | m-D14 | A-20 | H | H | H |
| 6178 | 0 | A-21 | m-D14 | A-21 | H | H | H |
| 6179 | 0 | A-22 | m-D14 | A-22 | H | H | H |
| 6180 | 0 | A-23 | m-D14 | A-23 | H | H | H |
| 6181 | 0 | A-24 | m-D14 | A-24 | H | H | H |
| 6182 | 0 | A-25 | m-D14 | A-25 | H | H | H |
| 6183 | 0 | A-26 | m-D14 | A-26 | H | H | H |
| 6184 | 0 | A-27 | m-D14 | A-27 | H | H | H |
| 6185 | 0 | A-28 | m-D14 | A-28 | H | H | H |
| 6186 | 0 | A-29 | m-D14 | A-29 | H | H | H |
| 6187 | 0 | A-30 | m-D14 | A-30 | H | H | H |
| 6188 | 0 | A-31 | m-D14 | A-31 | H | H | H |
| 6189 | 0 | A-32 | m-D14 | A-32 | H | H | H |
| 6190 | 0 | A-33 | m-D14 | A-33 | H | H | H |
| 6191 | 0 | A-34 | m-D14 | A-34 | H | H | H |
| 6192 | 0 | A-35 | m-D14 | A-35 | H | H | H |
| 6193 | 0 | A-36 | m-D14 | A-36 | H | H | H |
| 6194 | 0 | A-37 | m-D14 | A-37 | H | H | H |
| 6195 | 0 | A-38 | m-D14 | A-38 | H | H | H |
| 6196 | 0 | A-39 | m-D14 | A-39 | H | H | H |
| 6197 | 0 | A-40 | m-D14 | A-40 | H | H | H |
| 6198 | 0 | A-41 | m-D14 | A-41 | H | H | H |
| 6199 | 0 | A-42 | m-D14 | A-42 | H | H | H |
| 6200 | 0 | A-43 | m-D14 | A-43 | H | H | H |
| 6201 | 0 | A-44 | m-D14 | A-44 | H | H | H |
| 6202 | 0 | A-45 | m-D14 | A-45 | H | H | H |
| 6203 | 0 | A-46 | m-D14 | A-46 | H | H | H |
| 6204 | 0 | A-47 | m-D14 | A-47 | H | H | H |
| 6205 | 0 | A-1 | A-1 | m-D1 | H | H | H |
| 6206 | 0 | A-2 | A-2 | m-D1 | H | H | H |
| 6207 | 0 | A-3 | A-3 | m-D1 | H | H | H |
| 6208 | 0 | A-4 | A-4 | m-D1 | H | H | H |
| 6209 | 0 | A-5 | A-5 | m-D1 | H | H | H |
| 6210 | 0 | A-6 | A-6 | m-D1 | H | H | H |
| 6211 | 0 | A-7 | A-7 | m-D1 | H | H | H |
| 6212 | 0 | A-8 | A-8 | m-D1 | H | H | H |
| 6213 | 0 | A-9 | A-9 | m-D1 | H | H | H |
| 6214 | 0 | A-10 | A-10 | m-D1 | H | H | H |
| 6215 | 0 | A-11 | A-11 | m-D1 | H | H | H |
| 6216 | 0 | A-12 | A-12 | m-D1 | H | H | H |
| 6217 | 0 | A-13 | A-13 | m-D1 | H | H | H |
| 6218 | 0 | A-14 | A-14 | m-D1 | H | H | H |
| 6219 | 0 | A-15 | A-15 | m-D1 | H | H | H |
| 6220 | 0 | A-16 | A-16 | m-D1 | H | H | H |
| 6221 | 0 | A-17 | A-17 | m-D1 | H | H | H |
| 6222 | 0 | A-18 | A-18 | m-D1 | H | H | H |
| 6223 | 0 | A-19 | A-19 | m-D1 | H | H | H |
| 6224 | 0 | A-20 | A-20 | m-D1 | H | H | H |
| 6225 | 0 | A-21 | A-21 | m-D1 | H | H | H |
| 6226 | 0 | A-22 | A-22 | m-D1 | H | H | H |
| 6227 | 0 | A-23 | A-23 | m-D1 | H | H | H |
| 6228 | 0 | A-24 | A-24 | m-D1 | H | H | H |
| 6229 | 0 | A-25 | A-25 | m-D1 | H | H | H |
| 6230 | 0 | A-26 | A-26 | m-D1 | H | H | H |
| 6231 | 0 | A-27 | A-27 | m-D1 | H | H | H |
| 6232 | 0 | A-28 | A-28 | m-D1 | H | H | H |
| 6233 | 0 | A-29 | A-29 | m-D1 | H | H | H |
| 6234 | 0 | A-30 | A-30 | m-D1 | H | H | H |
| 6235 | 0 | A-31 | A-31 | m-D1 | H | H | H |
| 6236 | 0 | A-32 | A-32 | m-D1 | H | H | H |
| 6237 | 0 | A-33 | A-33 | m-D1 | H | H | H |
| 6238 | 0 | A-34 | A-34 | m-D1 | H | H | H |
| 6239 | 0 | A-35 | A-35 | m-D1 | H | H | H |
| 6240 | 0 | A-36 | A-36 | m-D1 | H | H | H |
| 6241 | 0 | A-37 | A-37 | m-D1 | H | H | H |
| 6242 | 0 | A-38 | A-38 | m-D1 | H | H | H |
| 6243 | 0 | A-39 | A-39 | m-D1 | H | H | H |
| 6244 | 0 | A-40 | A-40 | m-D1 | H | H | H |
| 6245 | 0 | A-41 | A-41 | m-D1 | H | H | H |
| 6246 | 0 | A-42 | A-42 | m-D1 | H | H | H |
| 6247 | 0 | A-43 | A-43 | m-D1 | H | H | H |
| 6248 | 0 | A-44 | A-44 | m-D1 | H | H | H |
| 6249 | 0 | A-45 | A-45 | m-D1 | H | H | H |
| 6250 | 0 | A-46 | A-46 | m-D1 | H | H | H |
| 6251 | 0 | A-47 | A-47 | m-D1 | H | H | H |
| 6252 | 0 | A-1 | A-1 | m-D2 | H | H | H |
| 6253 | 0 | A-2 | A-2 | m-D2 | H | H | H |
| 6254 | 0 | A-3 | A-3 | m-D2 | H | H | H |
| 6255 | 0 | A-4 | A-4 | m-D2 | H | H | H |
| 6256 | 0 | A-5 | A-5 | m-D2 | H | H | H |
| 6257 | 0 | A-6 | A-6 | m-D2 | H | H | H |
| 6258 | 0 | A-7 | A-7 | m-D2 | H | H | H |
| 6259 | 0 | A-8 | A-8 | m-D2 | H | H | H |
| 6260 | 0 | A-9 | A-9 | m-D2 | H | H | H |
| 6261 | 0 | A-10 | A-10 | m-D2 | H | H | H |
| 6262 | 0 | A-11 | A-11 | m-D2 | H | H | H |
| 6263 | 0 | A-12 | A-12 | m-D2 | H | H | H |
| 6264 | 0 | A-13 | A-13 | m-D2 | H | H | H |
| 6265 | 0 | A-14 | A-14 | m-D2 | H | H | H |
| 6266 | 0 | A-15 | A-15 | m-D2 | H | H | H |
| 6267 | 0 | A-16 | A-16 | m-D2 | H | H | H |
| 6268 | 0 | A-17 | A-17 | m-D2 | H | H | H |
| 6269 | 0 | A-18 | A-18 | m-D2 | H | H | H |
| 6270 | 0 | A-19 | A-19 | m-D2 | H | H | H |
| 6271 | 0 | A-20 | A-20 | m-D2 | H | H | H |
| 6272 | 0 | A-21 | A-21 | m-D2 | H | H | H |
| 6273 | 0 | A-22 | A-22 | m-D2 | H | H | H |
| 6274 | 0 | A-23 | A-23 | m-D2 | H | H | H |
| 6275 | 0 | A-24 | A-24 | m-D2 | H | H | H |
| 6276 | 0 | A-25 | A-25 | m-D2 | H | H | H |
| 6277 | 0 | A-26 | A-26 | m-D2 | H | H | H |
| 6278 | 0 | A-27 | A-27 | m-D2 | H | H | H |
| 6279 | 0 | A-28 | A-28 | m-D2 | H | H | H |
| 6280 | 0 | A-29 | A-29 | m-D2 | H | H | H |
| 6281 | 0 | A-30 | A-30 | m-D2 | H | H | H |
| 6282 | 0 | A-31 | A-31 | m-D2 | H | H | H |
| 6283 | 0 | A-32 | A-32 | m-D2 | H | H | H |
| 6284 | 0 | A-33 | A-33 | m-D2 | H | H | H |
| 6285 | 0 | A-34 | A-34 | m-D2 | H | H | H |
| 6286 | 0 | A-35 | A-35 | m-D2 | H | H | H |
| 6287 | 0 | A-36 | A-36 | m-D2 | H | H | H |
| 6288 | 0 | A-37 | A-37 | m-D2 | H | H | H |
| 6289 | 0 | A-38 | A-38 | m-D2 | H | H | H |
| 6290 | 0 | A-39 | A-39 | m-D2 | H | H | H |
| 6291 | 0 | A-40 | A-40 | m-D2 | H | H | H |
| 6292 | 0 | A-41 | A-41 | m-D2 | H | H | H |
| 6293 | 0 | A-42 | A-42 | m-D2 | H | H | H |
| 6294 | 0 | A-43 | A-43 | m-D2 | H | H | H |
| 6295 | 0 | A-44 | A-44 | m-D2 | H | H | H |
| 6296 | 0 | A-45 | A-45 | m-D2 | H | H | H |
| 6297 | 0 | A-46 | A-46 | m-D2 | H | H | H |
| 6298 | 0 | A-47 | A-47 | m-D2 | H | H | H |
| 6299 | 0 | A-1 | A-1 | m-D3 | H | H | H |
| 6300 | 0 | A-2 | A-2 | m-D3 | H | H | H |
| 6301 | 0 | A-3 | A-3 | m-D3 | H | H | H |
| 6302 | 0 | A-4 | A-4 | m-D3 | H | H | H |
| 6303 | 0 | A-5 | A-5 | m-D3 | H | H | H |
| 6304 | 0 | A-6 | A-6 | m-D3 | H | H | H |
| 6305 | 0 | A-7 | A-7 | m-D3 | H | H | H |
| 6306 | 0 | A-8 | A-8 | m-D3 | H | H | H |
| 6307 | 0 | A-9 | A-9 | m-D3 | H | H | H |
| 6308 | 0 | A-10 | A-10 | m-D3 | H | H | H |
| 6309 | 0 | A-11 | A-11 | m-D3 | H | H | H |
| 6310 | 0 | A-12 | A-12 | m-D3 | H | H | H |
| 6311 | 0 | A-13 | A-13 | m-D3 | H | H | H |
| 6312 | 0 | A-14 | A-14 | m-D3 | H | H | H |
| 6313 | 0 | A-15 | A-15 | m-D3 | H | H | H |
| 6314 | 0 | A-16 | A-16 | m-D3 | H | H | H |
| 6315 | 0 | A-17 | A-17 | m-D3 | H | H | H |
| 6316 | 0 | A-18 | A-18 | m-D3 | H | H | H |
| 6317 | 0 | A-19 | A-19 | m-D3 | H | H | H |
| 6318 | 0 | A-20 | A-20 | m-D3 | H | H | H |
| 6319 | 0 | A-21 | A-21 | m-D3 | H | H | H |
| 6320 | 0 | A-22 | A-22 | m-D3 | H | H | H |
| 6321 | 0 | A-23 | A-23 | m-D3 | H | H | H |
| 6322 | 0 | A-24 | A-24 | m-D3 | H | H | H |
| 6323 | 0 | A-25 | A-25 | m-D3 | H | H | H |
| 6324 | 0 | A-26 | A-26 | m-D3 | H | H | H |
| 6325 | 0 | A-27 | A-27 | m-D3 | H | H | H |
| 6326 | 0 | A-28 | A-28 | m-D3 | H | H | H |
| 6327 | 0 | A-29 | A-29 | m-D3 | H | H | H |
| 6328 | 0 | A-30 | A-30 | m-D3 | H | H | H |
| 6329 | 0 | A-31 | A-31 | m-D3 | H | H | H |
| 6330 | 0 | A-32 | A-32 | m-D3 | H | H | H |
| 6331 | 0 | A-33 | A-33 | m-D3 | H | H | H |
| 6332 | 0 | A-34 | A-34 | m-D3 | H | H | H |
| 6333 | 0 | A-35 | A-35 | m-D3 | H | H | H |
| 6334 | 0 | A-36 | A-36 | m-D3 | H | H | H |
| 6335 | 0 | A-37 | A-37 | m-D3 | H | H | H |
| 6336 | 0 | A-38 | A-38 | m-D3 | H | H | H |
| 6337 | 0 | A-39 | A-39 | m-D3 | H | H | H |
| 6338 | 0 | A-40 | A-40 | m-D3 | H | H | H |
| 6339 | 0 | A-41 | A-41 | m-D3 | H | H | H |
| 6340 | 0 | A-42 | A-42 | m-D3 | H | H | H |
| 6341 | 0 | A-43 | A-43 | m-D3 | H | H | H |
| 6342 | 0 | A-44 | A-44 | m-D3 | H | H | H |
| 6343 | 0 | A-45 | A-45 | m-D3 | H | H | H |
| 6344 | 0 | A-46 | A-46 | m-D3 | H | H | H |
| 6345 | 0 | A-47 | A-47 | m-D3 | H | H | H |
| 6346 | 0 | A-1 | A-1 | m-D5 | H | H | H |
| 6347 | 0 | A-2 | A-2 | m-D5 | H | H | H |
| 6348 | 0 | A-3 | A-3 | m-D5 | H | H | H |
| 6349 | 0 | A-4 | A-4 | m-D5 | H | H | H |
| 6350 | 0 | A-5 | A-5 | m-D5 | H | H | H |
| 6351 | 0 | A-6 | A-6 | m-D5 | H | H | H |
| 6352 | 0 | A-7 | A-7 | m-D5 | H | H | H |
| 6353 | 0 | A-8 | A-8 | m-D5 | H | H | H |
| 6354 | 0 | A-9 | A-9 | m-D5 | H | H | H |
| 6355 | 0 | A-10 | A-10 | m-D5 | H | H | H |
| 6356 | 0 | A-11 | A-11 | m-D5 | H | H | H |
| 6357 | 0 | A-12 | A-12 | m-D5 | H | H | H |
| 6358 | 0 | A-13 | A-13 | m-D5 | H | H | H |
| 6359 | 0 | A-14 | A-14 | m-D5 | H | H | H |
| 6360 | 0 | A-15 | A-15 | m-D5 | H | H | H |
| 6361 | 0 | A-16 | A-16 | m-D5 | H | H | H |
| 6362 | 0 | A-17 | A-17 | m-D5 | H | H | H |
| 6363 | 0 | A-18 | A-18 | m-D5 | H | H | H |
| 6364 | 0 | A-19 | A-19 | m-D5 | H | H | H |
| 6365 | 0 | A-20 | A-20 | m-D5 | H | H | H |
| 6366 | 0 | A-21 | A-21 | m-D5 | H | H | H |
| 6367 | 0 | A-22 | A-22 | m-D5 | H | H | H |
| 6368 | 0 | A-23 | A-23 | m-D5 | H | H | H |
| 6369 | 0 | A-24 | A-24 | m-D5 | H | H | H |
| 6370 | 0 | A-25 | A-25 | m-D5 | H | H | H |
| 6371 | 0 | A-26 | A-26 | m-D5 | H | H | H |
| 6372 | 0 | A-27 | A-27 | m-D5 | H | H | H |
| 6373 | 0 | A-28 | A-28 | m-D5 | H | H | H |
| 6374 | 0 | A-29 | A-29 | m-D5 | H | H | H |
| 6375 | 0 | A-30 | A-30 | m-D5 | H | H | H |
| 6376 | 0 | A-31 | A-31 | m-D5 | H | H | H |
| 6377 | 0 | A-32 | A-32 | m-D5 | H | H | H |
| 6378 | 0 | A-33 | A-33 | m-D5 | H | H | H |
| 6379 | 0 | A-34 | A-34 | m-D5 | H | H | H |
| 6380 | 0 | A-35 | A-35 | m-D5 | H | H | H |
| 6381 | 0 | A-36 | A-36 | m-D5 | H | H | H |
| 6382 | 0 | A-37 | A-37 | m-D5 | H | H | H |
| 6383 | 0 | A-38 | A-38 | m-D5 | H | H | H |
| 6384 | 0 | A-39 | A-39 | m-D5 | H | H | H |
| 6385 | 0 | A-40 | A-40 | m-D5 | H | H | H |
| 6386 | 0 | A-41 | A-41 | m-D5 | H | H | H |
| 6387 | 0 | A-42 | A-42 | m-D5 | H | H | H |
| 6388 | 0 | A-43 | A-43 | m-D5 | H | H | H |
| 6389 | 0 | A-44 | A-44 | m-D5 | H | H | H |
| 6390 | 0 | A-45 | A-45 | m-D5 | H | H | H |
| 6391 | 0 | A-46 | A-46 | m-D5 | H | H | H |
| 6392 | 0 | A-47 | A-47 | m-D5 | H | H | H |
| 6393 | 0 | A-1 | A-1 | m-D9 | H | H | H |
| 6394 | 0 | A-2 | A-2 | m-D9 | H | H | H |
| 6395 | 0 | A-3 | A-3 | m-D9 | H | H | H |
| 6396 | 0 | A-4 | A-4 | m-D9 | H | H | H |
| 6397 | 0 | A-5 | A-5 | m-D9 | H | H | H |
| 6398 | 0 | A-6 | A-6 | m-D9 | H | H | H |
| 6399 | 0 | A-7 | A-7 | m-D9 | H | H | H |
| 6400 | 0 | A-8 | A-8 | m-D9 | H | H | H |
| 6401 | 0 | A-9 | A-9 | m-D9 | H | H | H |
| 6402 | 0 | A-10 | A-10 | m-D9 | H | H | H |
| 6403 | 0 | A-11 | A-11 | m-D9 | H | H | H |
| 6404 | 0 | A-12 | A-12 | m-D9 | H | H | H |
| 6405 | 0 | A-13 | A-13 | m-D9 | H | H | H |
| 6406 | 0 | A-14 | A-14 | m-D9 | H | H | H |
| 6407 | 0 | A-15 | A-15 | m-D9 | H | H | H |
| 6408 | 0 | A-16 | A-16 | m-D9 | H | H | H |
| 6409 | 0 | A-17 | A-17 | m-D9 | H | H | H |
| 6410 | 0 | A-18 | A-18 | m-D9 | H | H | H |
| 6411 | 0 | A-19 | A-19 | m-D9 | H | H | H |
| 6412 | 0 | A-20 | A-20 | m-D9 | H | H | H |
| 6413 | 0 | A-21 | A-21 | m-D9 | H | H | H |
| 6414 | 0 | A-22 | A-22 | m-D9 | H | H | H |
| 6415 | 0 | A-23 | A-23 | m-D9 | H | H | H |
| 6416 | 0 | A-24 | A-24 | m-D9 | H | H | H |
| 6417 | 0 | A-25 | A-25 | m-D9 | H | H | H |
| 6418 | 0 | A-26 | A-26 | m-D9 | H | H | H |
| 6419 | 0 | A-27 | A-27 | m-D9 | H | H | H |
| 6420 | 0 | A-28 | A-28 | m-D9 | H | H | H |
| 6421 | 0 | A-29 | A-29 | m-D9 | H | H | H |
| 6422 | 0 | A-30 | A-30 | m-D9 | H | H | H |
| 6423 | 0 | A-31 | A-31 | m-D9 | H | H | H |
| 6424 | 0 | A-32 | A-32 | m-D9 | H | H | H |
| 6425 | 0 | A-33 | A-33 | m-D9 | H | H | H |
| 6426 | 0 | A-34 | A-34 | m-D9 | H | H | H |
| 6427 | 0 | A-35 | A-35 | m-D9 | H | H | H |
| 6428 | 0 | A-36 | A-36 | m-D9 | H | H | H |
| 6429 | 0 | A-37 | A-37 | m-D9 | H | H | H |
| 6430 | 0 | A-38 | A-38 | m-D9 | H | H | H |
| 6431 | 0 | A-39 | A-39 | m-D9 | H | H | H |
| 6432 | 0 | A-40 | A-40 | m-D9 | H | H | H |
| 6433 | 0 | A-41 | A-41 | m-D9 | H | H | H |
| 6434 | 0 | A-42 | A-42 | m-D9 | H | H | H |
| 6435 | 0 | A-43 | A-43 | m-D9 | H | H | H |
| 6436 | 0 | A-44 | A-44 | m-D9 | H | H | H |
| 6437 | 0 | A-45 | A-45 | m-D9 | H | H | H |
| 6438 | 0 | A-46 | A-46 | m-D9 | H | H | H |
| 6439 | 0 | A-47 | A-47 | m-D9 | H | H | H |
| 6440 | 0 | A-1 | A-1 | m-D11 | H | H | H |
| 6441 | 0 | A-2 | A-2 | m-D11 | H | H | H |
| 6442 | 0 | A-3 | A-3 | m-D11 | H | H | H |
| 6443 | 0 | A-4 | A-4 | m-D11 | H | H | H |
| 6444 | 0 | A-5 | A-5 | m-D11 | H | H | H |
| 6445 | 0 | A-6 | A-6 | m-D11 | H | H | H |
| 6446 | 0 | A-7 | A-7 | m-D11 | H | H | H |
| 6447 | 0 | A-8 | A-8 | m-D11 | H | H | H |
| 6448 | 0 | A-9 | A-9 | m-D11 | H | H | H |
| 6449 | 0 | A-10 | A-10 | m-D11 | H | H | H |
| 6450 | 0 | A-11 | A-11 | m-D11 | H | H | H |
| 6451 | 0 | A-12 | A-12 | m-D11 | H | H | H |
| 6452 | 0 | A-13 | A-13 | m-D11 | H | H | H |
| 6453 | 0 | A-14 | A-14 | m-D11 | H | H | H |
| 6454 | 0 | A-15 | A-15 | m-D11 | H | H | H |
| 6455 | 0 | A-16 | A-16 | m-D11 | H | H | H |
| 6456 | 0 | A-17 | A-17 | m-D11 | H | H | H |
| 6457 | 0 | A-18 | A-18 | m-D11 | H | H | H |
| 6458 | 0 | A-19 | A-19 | m-D11 | H | H | H |
| 6459 | 0 | A-20 | A-20 | m-D11 | H | H | H |
| 6460 | 0 | A-21 | A-21 | m-D11 | H | H | H |
| 6461 | 0 | A-22 | A-22 | m-D11 | H | H | H |
| 6462 | 0 | A-23 | A-23 | m-D11 | H | H | H |
| 6463 | 0 | A-24 | A-24 | m-D11 | H | H | H |
| 6464 | 0 | A-25 | A-25 | m-D11 | H | H | H |
| 6465 | 0 | A-26 | A-26 | m-D11 | H | H | H |
| 6466 | 0 | A-27 | A-27 | m-D11 | H | H | H |
| 6467 | 0 | A-28 | A-28 | m-D11 | H | H | H |
| 6468 | 0 | A-29 | A-29 | m-D11 | H | H | H |
| 6469 | 0 | A-30 | A-30 | m-D11 | H | H | H |
| 6470 | 0 | A-31 | A-31 | m-D11 | H | H | H |
| 6471 | 0 | A-32 | A-32 | m-D11 | H | H | H |
| 6472 | 0 | A-33 | A-33 | m-D11 | H | H | H |
| 6473 | 0 | A-34 | A-34 | m-D11 | H | H | H |
| 6474 | 0 | A-35 | A-35 | m-D11 | H | H | H |
| 6475 | 0 | A-36 | A-36 | m-D11 | H | H | H |
| 6476 | 0 | A-37 | A-37 | m-D11 | H | H | H |
| 6477 | 0 | A-38 | A-38 | m-D11 | H | H | H |
| 6478 | 0 | A-39 | A-39 | m-D11 | H | H | H |
| 6479 | 0 | A-40 | A-40 | m-D11 | H | H | H |
| 6480 | 0 | A-41 | A-41 | m-D11 | H | H | H |
| 6481 | 0 | A-42 | A-42 | m-D11 | H | H | H |
| 6482 | 0 | A-43 | A-43 | m-D11 | H | H | H |
| 6483 | 0 | A-44 | A-44 | m-D11 | H | H | H |
| 6484 | 0 | A-45 | A-45 | m-D11 | H | H | H |
| 6485 | 0 | A-46 | A-46 | m-D11 | H | H | H |
| 6486 | 0 | A-47 | A-47 | m-D11 | H | H | H |
| 6487 | 0 | A-1 | A-1 | m-D12 | H | H | H |
| 6488 | 0 | A-2 | A-2 | m-D12 | H | H | H |
| 6489 | 0 | A-3 | A-3 | m-D12 | H | H | H |
| 6490 | 0 | A-4 | A-4 | m-D12 | H | H | H |
| 6491 | 0 | A-5 | A-5 | m-D12 | H | H | H |
| 6492 | 0 | A-6 | A-6 | m-D12 | H | H | H |
| 6493 | 0 | A-7 | A-7 | m-D12 | H | H | H |
| 6494 | 0 | A-8 | A-8 | m-D12 | H | H | H |
| 6495 | 0 | A-9 | A-9 | m-D12 | H | H | H |
| 6496 | 0 | A-10 | A-10 | m-D12 | H | H | H |
| 6497 | 0 | A-11 | A-11 | m-D12 | H | H | H |
| 6498 | 0 | A-12 | A-12 | m-D12 | H | H | H |
| 6499 | 0 | A-13 | A-13 | m-D12 | H | H | H |
| 6500 | 0 | A-14 | A-14 | m-D12 | H | H | H |
| 6501 | 0 | A-15 | A-15 | m-D12 | H | H | H |
| 6502 | 0 | A-16 | A-16 | m-D12 | H | H | H |
| 6503 | 0 | A-17 | A-17 | m-D12 | H | H | H |
| 6504 | 0 | A-18 | A-18 | m-D12 | H | H | H |
| 6505 | 0 | A-19 | A-19 | m-D12 | H | H | H |
| 6506 | 0 | A-20 | A-20 | m-D12 | H | H | H |
| 6507 | 0 | A-21 | A-21 | m-D12 | H | H | H |
| 6508 | 0 | A-22 | A-22 | m-D12 | H | H | H |
| 6509 | 0 | A-23 | A-23 | m-D12 | H | H | H |
| 6510 | 0 | A-24 | A-24 | m-D12 | H | H | H |
| 6511 | 0 | A-25 | A-25 | m-D12 | H | H | H |
| 6512 | 0 | A-26 | A-26 | m-D12 | H | H | H |
| 6513 | 0 | A-27 | A-27 | m-D12 | H | H | H |
| 6514 | 0 | A-28 | A-28 | m-D12 | H | H | H |
| 6515 | 0 | A-29 | A-29 | m-D12 | H | H | H |
| 6516 | 0 | A-30 | A-30 | m-D12 | H | H | H |
| 6517 | 0 | A-31 | A-31 | m-D12 | H | H | H |
| 6518 | 0 | A-32 | A-32 | m-D12 | H | H | H |
| 6519 | 0 | A-33 | A-33 | m-D12 | H | H | H |
| 6520 | 0 | A-34 | A-34 | m-D12 | H | H | H |
| 6521 | 0 | A-35 | A-35 | m-D12 | H | H | H |
| 6522 | 0 | A-36 | A-36 | m-D12 | H | H | H |
| 6523 | 0 | A-37 | A-37 | m-D12 | H | H | H |
| 6524 | 0 | A-38 | A-38 | m-D12 | H | H | H |
| 6525 | 0 | A-39 | A-39 | m-D12 | H | H | H |
| 6526 | 0 | A-40 | A-40 | m-D12 | H | H | H |
| 6527 | 0 | A-41 | A-41 | m-D12 | H | H | H |
| 6528 | 0 | A-42 | A-42 | m-D12 | H | H | H |
| 6529 | 0 | A-43 | A-43 | m-D12 | H | H | H |
| 6530 | 0 | A-44 | A-44 | m-D12 | H | H | H |
| 6531 | 0 | A-45 | A-45 | m-D12 | H | H | H |
| 6532 | 0 | A-46 | A-46 | m-D12 | H | H | H |
| 6533 | 0 | A-47 | A-47 | m-D12 | H | H | H |
| 6534 | 0 | A-1 | A-1 | m-D13 | H | H | H |
| 6535 | 0 | A-2 | A-2 | m-D13 | H | H | H |
| 6536 | 0 | A-3 | A-3 | m-D13 | H | H | H |
| 6537 | 0 | A-4 | A-4 | m-D13 | H | H | H |
| 6538 | 0 | A-5 | A-5 | m-D13 | H | H | H |
| 6539 | 0 | A-6 | A-6 | m-D13 | H | H | H |
| 6540 | 0 | A-7 | A-7 | m-D13 | H | H | H |
| 6541 | 0 | A-8 | A-8 | m-D13 | H | H | H |
| 6542 | 0 | A-9 | A-9 | m-D13 | H | H | H |
| 6543 | 0 | A-10 | A-10 | m-D13 | H | H | H |
| 6544 | 0 | A-11 | A-11 | m-D13 | H | H | H |
| 6545 | 0 | A-12 | A-12 | m-D13 | H | H | H |
| 6546 | 0 | A-13 | A-13 | m-D13 | H | H | H |
| 6547 | 0 | A-14 | A-14 | m-D13 | H | H | H |
| 6548 | 0 | A-15 | A-15 | m-D13 | H | H | H |
| 6549 | 0 | A-16 | A-16 | m-D13 | H | H | H |
| 6550 | 0 | A-17 | A-17 | m-D13 | H | H | H |
| 6551 | 0 | A-18 | A-18 | m-D13 | H | H | H |
| 6552 | 0 | A-19 | A-19 | m-D13 | H | H | H |
| 6553 | 0 | A-20 | A-20 | m-D13 | H | H | H |
| 6554 | 0 | A-21 | A-21 | m-D13 | H | H | H |
| 6555 | 0 | A-22 | A-22 | m-D13 | H | H | H |
| 6556 | 0 | A-23 | A-23 | m-D13 | H | H | H |
| 6557 | 0 | A-24 | A-24 | m-D13 | H | H | H |
| 6558 | 0 | A-25 | A-25 | m-D13 | H | H | H |
| 6559 | 0 | A-26 | A-26 | m-D13 | H | H | H |
| 6560 | 0 | A-27 | A-27 | m-D13 | H | H | H |
| 6561 | 0 | A-28 | A-28 | m-D13 | H | H | H |
| 6562 | 0 | A-29 | A-29 | m-D13 | H | H | H |
| 6563 | 0 | A-30 | A-30 | m-D13 | H | H | H |
| 6564 | 0 | A-31 | A-31 | m-D13 | H | H | H |
| 6565 | 0 | A-32 | A-32 | m-D13 | H | H | H |
| 6566 | 0 | A-33 | A-33 | m-D13 | H | H | H |
| 6567 | 0 | A-34 | A-34 | m-D13 | H | H | H |
| 6568 | 0 | A-35 | A-35 | m-D13 | H | H | H |
| 6569 | 0 | A-36 | A-36 | m-D13 | H | H | H |
| 6570 | 0 | A-37 | A-37 | m-D13 | H | H | H |
| 6571 | 0 | A-38 | A-38 | m-D13 | H | H | H |
| 6572 | 0 | A-39 | A-39 | m-D13 | H | H | H |
| 6573 | 0 | A-40 | A-40 | m-D13 | H | H | H |
| 6574 | 0 | A-41 | A-41 | m-D13 | H | H | H |
| 6575 | 0 | A-42 | A-42 | m-D13 | H | H | H |
| 6576 | 0 | A-43 | A-43 | m-D13 | H | H | H |
| 6577 | 0 | A-44 | A-44 | m-D13 | H | H | H |
| 6578 | 0 | A-45 | A-45 | m-D13 | H | H | H |
| 6579 | 0 | A-46 | A-46 | m-D13 | H | H | H |
| 6580 | 0 | A-47 | A-47 | m-D13 | H | H | H |
| 6581 | 0 | A-1 | A-1 | m-D14 | H | H | H |
| 6582 | 0 | A-2 | A-2 | m-D14 | H | H | H |
| 6583 | 0 | A-3 | A-3 | m-D14 | H | H | H |
| 6584 | 0 | A-4 | A-4 | m-D14 | H | H | H |
| 6585 | 0 | A-5 | A-5 | m-D14 | H | H | H |
| 6586 | 0 | A-6 | A-6 | m-D14 | H | H | H |
| 6587 | 0 | A-7 | A-7 | m-D14 | H | H | H |
| 6588 | 0 | A-8 | A-8 | m-D14 | H | H | H |
| 6589 | 0 | A-9 | A-9 | m-D14 | H | H | H |
| 6590 | 0 | A-10 | A-10 | m-D14 | H | H | H |
| 6591 | 0 | A-11 | A-11 | m-D14 | H | H | H |
| 6592 | 0 | A-12 | A-12 | m-D14 | H | H | H |
| 6593 | 0 | A-13 | A-13 | m-D14 | H | H | H |
| 6594 | 0 | A-14 | A-14 | m-D14 | H | H | H |
| 6595 | 0 | A-15 | A-15 | m-D14 | H | H | H |
| 6596 | 0 | A-16 | A-16 | m-D14 | H | H | H |
| 6597 | 0 | A-17 | A-17 | m-D14 | H | H | H |
| 6598 | 0 | A-18 | A-18 | m-D14 | H | H | H |
| 6599 | 0 | A-19 | A-19 | m-D14 | H | H | H |
| 6600 | 0 | A-20 | A-20 | m-D14 | H | H | H |
| 6601 | 0 | A-21 | A-21 | m-D14 | H | H | H |
| 6602 | 0 | A-22 | A-22 | m-D14 | H | H | H |
| 6603 | 0 | A-23 | A-23 | m-D14 | H | H | H |
| 6604 | 0 | A-24 | A-24 | m-D14 | H | H | H |
| 6605 | 0 | A-25 | A-25 | m-D14 | H | H | H |
| 6606 | 0 | A-26 | A-26 | m-D14 | H | H | H |
| 6607 | 0 | A-27 | A-27 | m-D14 | H | H | H |
| 6608 | 0 | A-28 | A-28 | m-D14 | H | H | H |
| 6609 | 0 | A-29 | A-29 | m-D14 | H | H | H |
| 6610 | 0 | A-30 | A-30 | m-D14 | H | H | H |
| 6611 | 0 | A-31 | A-31 | m-D14 | H | H | H |
| 6612 | 0 | A-32 | A-32 | m-D14 | H | H | H |
| 6613 | 0 | A-33 | A-33 | m-D14 | H | H | H |
| 6614 | 0 | A-34 | A-34 | m-D14 | H | H | H |
| 6615 | 0 | A-35 | A-35 | m-D14 | H | H | H |
| 6616 | 0 | A-36 | A-36 | m-D14 | H | H | H |
| 6617 | 0 | A-37 | A-37 | m-D14 | H | H | H |
| 6618 | 0 | A-38 | A-38 | m-D14 | H | H | H |
| 6619 | 0 | A-39 | A-39 | m-D14 | H | H | H |
| 6620 | 0 | A-40 | A-40 | m-D14 | H | H | H |
| 6621 | 0 | A-41 | A-41 | m-D14 | H | H | H |
| 6622 | 0 | A-42 | A-42 | m-D14 | H | H | H |
| 6623 | 0 | A-43 | A-43 | m-D14 | H | H | H |
| 6624 | 0 | A-44 | A-44 | m-D14 | H | H | H |
| 6625 | 0 | A-45 | A-45 | m-D14 | H | H | H |
| 6626 | 0 | A-46 | A-46 | m-D14 | H | H | H |
| 6627 | 0 | A-47 | A-47 | m-D14 | H | H | H |
| 6628 | 0 | m-D1 | A-1 | m-D1 | H | H | H |
| 6629 | 0 | m-D1 | A-2 | m-D1 | H | H | H |
| 6630 | 0 | m-D1 | A-3 | m-D1 | H | H | H |
| 6631 | 0 | m-D1 | A-4 | m-D1 | H | H | H |
| 6632 | 0 | m-D1 | A-5 | m-D1 | H | H | H |
| 6633 | 0 | m-D1 | A-6 | m-D1 | H | H | H |
| 6634 | 0 | m-D1 | A-7 | m-D1 | H | H | H |
| 6635 | 0 | m-D1 | A-8 | m-D1 | H | H | H |
| 6636 | 0 | m-D1 | A-9 | m-D1 | H | H | H |
| 6637 | 0 | m-D1 | A-10 | m-D1 | H | H | H |
| 6638 | 0 | m-D1 | A-11 | m-D1 | H | H | H |
| 6639 | 0 | m-D1 | A-12 | m-D1 | H | H | H |
| 6640 | 0 | m-D1 | A-13 | m-D1 | H | H | H |
| 6641 | 0 | m-D1 | A-14 | m-D1 | H | H | H |
| 6642 | 0 | m-D1 | A-15 | m-D1 | H | H | H |
| 6643 | 0 | m-D1 | A-16 | m-D1 | H | H | H |
| 6644 | 0 | m-D1 | A-17 | m-D1 | H | H | H |
| 6645 | 0 | m-D1 | A-18 | m-D1 | H | H | H |
| 6646 | 0 | m-D1 | A-19 | m-D1 | H | H | H |
| 6647 | 0 | m-D1 | A-20 | m-D1 | H | H | H |
| 6648 | 0 | m-D1 | A-21 | m-D1 | H | H | H |
| 6649 | 0 | m-D1 | A-22 | m-D1 | H | H | H |
| 6650 | 0 | m-D1 | A-23 | m-D1 | H | H | H |
| 6651 | 0 | m-D1 | A-24 | m-D1 | H | H | H |
| 6652 | 0 | m-D1 | A-25 | m-D1 | H | H | H |
| 6653 | 0 | m-D1 | A-26 | m-D1 | H | H | H |
| 6654 | 0 | m-D1 | A-27 | m-D1 | H | H | H |
| 6655 | 0 | m-D1 | A-28 | m-D1 | H | H | H |
| 6656 | 0 | m-D1 | A-29 | m-D1 | H | H | H |
| 6657 | 0 | m-D1 | A-30 | m-D1 | H | H | H |
| 6658 | 0 | m-D1 | A-31 | m-D1 | H | H | H |
| 6659 | 0 | m-D1 | A-32 | m-D1 | H | H | H |
| 6660 | 0 | m-D1 | A-33 | m-D1 | H | H | H |
| 6661 | 0 | m-D1 | A-34 | m-D1 | H | H | H |
| 6662 | 0 | m-D1 | A-35 | m-D1 | H | H | H |
| 6663 | 0 | m-D1 | A-36 | m-D1 | H | H | H |
| 6664 | 0 | m-D1 | A-37 | m-D1 | H | H | H |
| 6665 | 0 | m-D1 | A-38 | m-D1 | H | H | H |
| 6666 | 0 | m-D1 | A-39 | m-D1 | H | H | H |
| 6667 | 0 | m-D1 | A-40 | m-D1 | H | H | H |
| 6668 | 0 | m-D1 | A-41 | m-D1 | H | H | H |
| 6669 | 0 | m-D1 | A-42 | m-D1 | H | H | H |
| 6670 | 0 | m-D1 | A-43 | m-D1 | H | H | H |
| 6671 | 0 | m-D1 | A-44 | m-D1 | H | H | H |
| 6672 | 0 | m-D1 | A-45 | m-D1 | H | H | H |
| 6673 | 0 | m-D1 | A-46 | m-D1 | H | H | H |
| 6674 | 0 | m-D1 | A-47 | m-D1 | H | H | H |
| 6675 | 0 | m-D2 | A-1 | m-D2 | H | H | H |
| 6676 | 0 | m-D2 | A-2 | m-D2 | H | H | H |
| 6677 | 0 | m-D2 | A-3 | m-D2 | H | H | H |
| 6678 | 0 | m-D2 | A-4 | m-D2 | H | H | H |
| 6679 | 0 | m-D2 | A-5 | m-D2 | H | H | H |
| 6680 | 0 | m-D2 | A-6 | m-D2 | H | H | H |
| 6681 | 0 | m-D2 | A-7 | m-D2 | H | H | H |
| 6682 | 0 | m-D2 | A-8 | m-D2 | H | H | H |
| 6683 | 0 | m-D2 | A-9 | m-D2 | H | H | H |
| 6684 | 0 | m-D2 | A-10 | m-D2 | H | H | H |
| 6685 | 0 | m-D2 | A-11 | m-D2 | H | H | H |
| 6686 | 0 | m-D2 | A-12 | m-D2 | H | H | H |
| 6687 | 0 | m-D2 | A-13 | m-D2 | H | H | H |
| 6688 | 0 | m-D2 | A-14 | m-D2 | H | H | H |
| 6689 | 0 | m-D2 | A-15 | m-D2 | H | H | H |
| 6690 | 0 | m-D2 | A-16 | m-D2 | H | H | H |
| 6691 | 0 | m-D2 | A-17 | m-D2 | H | H | H |
| 6692 | 0 | m-D2 | A-18 | m-D2 | H | H | H |
| 6693 | 0 | m-D2 | A-19 | m-D2 | H | H | H |
| 6694 | 0 | m-D2 | A-20 | m-D2 | H | H | H |
| 6695 | 0 | m-D2 | A-21 | m-D2 | H | H | H |
| 6696 | 0 | m-D2 | A-22 | m-D2 | H | H | H |
| 6697 | 0 | m-D2 | A-23 | m-D2 | H | H | H |
| 6698 | 0 | m-D2 | A-24 | m-D2 | H | H | H |
| 6699 | 0 | m-D2 | A-25 | m-D2 | H | H | H |
| 6700 | 0 | m-D2 | A-26 | m-D2 | H | H | H |
| 6701 | 0 | m-D2 | A-27 | m-D2 | H | H | H |
| 6702 | 0 | m-D2 | A-28 | m-D2 | H | H | H |
| 6703 | 0 | m-D2 | A-29 | m-D2 | H | H | H |
| 6704 | 0 | m-D2 | A-30 | m-D2 | H | H | H |
| 6705 | 0 | m-D2 | A-31 | m-D2 | H | H | H |
| 6706 | 0 | m-D2 | A-32 | m-D2 | H | H | H |
| 6707 | 0 | m-D2 | A-33 | m-D2 | H | H | H |
| 6708 | 0 | m-D2 | A-34 | m-D2 | H | H | H |
| 6709 | 0 | m-D2 | A-35 | m-D2 | H | H | H |
| 6710 | 0 | m-D2 | A-36 | m-D2 | H | H | H |
| 6711 | 0 | m-D2 | A-37 | m-D2 | H | H | H |
| 6712 | 0 | m-D2 | A-38 | m-D2 | H | H | H |
| 6713 | 0 | m-D2 | A-39 | m-D2 | H | H | H |
| 6714 | 0 | m-D2 | A-40 | m-D2 | H | H | H |
| 6715 | 0 | m-D2 | A-41 | m-D2 | H | H | H |
| 6716 | 0 | m-D2 | A-42 | m-D2 | H | H | H |
| 6717 | 0 | m-D2 | A-43 | m-D2 | H | H | H |
| 6718 | 0 | m-D2 | A-44 | m-D2 | H | H | H |
| 6719 | 0 | m-D2 | A-45 | m-D2 | H | H | H |
| 6720 | 0 | m-D2 | A-46 | m-D2 | H | H | H |
| 6721 | 0 | m-D2 | A-47 | m-D2 | H | H | H |
| 6722 | 0 | m-D3 | A-1 | m-D3 | H | H | H |
| 6723 | 0 | m-D3 | A-2 | m-D3 | H | H | H |
| 6724 | 0 | m-D3 | A-3 | m-D3 | H | H | H |
| 6725 | 0 | m-D3 | A-4 | m-D3 | H | H | H |
| 6726 | 0 | m-D3 | A-5 | m-D3 | H | H | H |
| 6727 | 0 | m-D3 | A-6 | m-D3 | H | H | H |
| 6728 | 0 | m-D3 | A-7 | m-D3 | H | H | H |
| 6729 | 0 | m-D3 | A-8 | m-D3 | H | H | H |
| 6730 | 0 | m-D3 | A-9 | m-D3 | H | H | H |
| 6731 | 0 | m-D3 | A-10 | m-D3 | H | H | H |
| 6732 | 0 | m-D3 | A-11 | m-D3 | H | H | H |
| 6733 | 0 | m-D3 | A-12 | m-D3 | H | H | H |
| 6734 | 0 | m-D3 | A-13 | m-D3 | H | H | H |
| 6735 | 0 | m-D3 | A-14 | m-D3 | H | H | H |
| 6736 | 0 | m-D3 | A-15 | m-D3 | H | H | H |
| 6737 | 0 | m-D3 | A-16 | m-D3 | H | H | H |
| 6738 | 0 | m-D3 | A-17 | m-D3 | H | H | H |
| 6739 | 0 | m-D3 | A-18 | m-D3 | H | H | H |
| 6740 | 0 | m-D3 | A-19 | m-D3 | H | H | H |
| 6741 | 0 | m-D3 | A-20 | m-D3 | H | H | H |
| 6742 | 0 | m-D3 | A-21 | m-D3 | H | H | H |
| 6743 | 0 | m-D3 | A-22 | m-D3 | H | H | H |
| 6744 | 0 | m-D3 | A-23 | m-D3 | H | H | H |
| 6745 | 0 | m-D3 | A-24 | m-D3 | H | H | H |
| 6746 | 0 | m-D3 | A-25 | m-D3 | H | H | H |
| 6747 | 0 | m-D3 | A-26 | m-D3 | H | H | H |
| 6748 | 0 | m-D3 | A-27 | m-D3 | H | H | H |
| 6749 | 0 | m-D3 | A-28 | m-D3 | H | H | H |
| 6750 | 0 | m-D3 | A-29 | m-D3 | H | H | H |
| 6751 | 0 | m-D3 | A-30 | m-D3 | H | H | H |
| 6752 | 0 | m-D3 | A-31 | m-D3 | H | H | H |
| 6753 | 0 | m-D3 | A-32 | m-D3 | H | H | H |
| 6754 | 0 | m-D3 | A-33 | m-D3 | H | H | H |
| 6755 | 0 | m-D3 | A-34 | m-D3 | H | H | H |
| 6756 | 0 | m-D3 | A-35 | m-D3 | H | H | H |
| 6757 | 0 | m-D3 | A-36 | m-D3 | H | H | H |
| 6758 | 0 | m-D3 | A-37 | m-D3 | H | H | H |
| 6759 | 0 | m-D3 | A-38 | m-D3 | H | H | H |
| 6760 | 0 | m-D3 | A-39 | m-D3 | H | H | H |
| 6761 | 0 | m-D3 | A-40 | m-D3 | H | H | H |
| 6762 | 0 | m-D3 | A-41 | m-D3 | H | H | H |
| 6763 | 0 | m-D3 | A-42 | m-D3 | H | H | H |
| 6764 | 0 | m-D3 | A-43 | m-D3 | H | H | H |
| 6765 | 0 | m-D3 | A-44 | m-D3 | H | H | H |
| 6766 | 0 | m-D3 | A-45 | m-D3 | H | H | H |
| 6767 | 0 | m-D3 | A-46 | m-D3 | H | H | H |
| 6768 | 0 | m-D3 | A-47 | m-D3 | H | H | H |
| 6769 | 0 | m-D5 | A-1 | m-D5 | H | H | H |
| 6770 | 0 | m-D5 | A-2 | m-D5 | H | H | H |
| 6771 | 0 | m-D5 | A-3 | m-D5 | H | H | H |
| 6772 | 0 | m-D5 | A-4 | m-D5 | H | H | H |
| 6773 | 0 | m-D5 | A-5 | m-D5 | H | H | H |
| 6774 | 0 | m-D5 | A-6 | m-D5 | H | H | H |
| 6775 | 0 | m-D5 | A-7 | m-D5 | H | H | H |
| 6776 | 0 | m-D5 | A-8 | m-D5 | H | H | H |
| 6777 | 0 | m-D5 | A-9 | m-D5 | H | H | H |
| 6778 | 0 | m-D5 | A-10 | m-D5 | H | H | H |
| 6779 | 0 | m-D5 | A-11 | m-D5 | H | H | H |
| 6780 | 0 | m-D5 | A-12 | m-D5 | H | H | H |
| 6781 | 0 | m-D5 | A-13 | m-D5 | H | H | H |
| 6782 | 0 | m-D5 | A-14 | m-D5 | H | H | H |
| 6783 | 0 | m-D5 | A-15 | m-D5 | H | H | H |
| 6784 | 0 | m-D5 | A-16 | m-D5 | H | H | H |
| 6785 | 0 | m-D5 | A-17 | m-D5 | H | H | H |
| 6786 | 0 | m-D5 | A-18 | m-D5 | H | H | H |
| 6787 | 0 | m-D5 | A-19 | m-D5 | H | H | H |
| 6788 | 0 | m-D5 | A-20 | m-D5 | H | H | H |
| 6789 | 0 | m-D5 | A-21 | m-D5 | H | H | H |
| 6790 | 0 | m-D5 | A-22 | m-D5 | H | H | H |
| 6791 | 0 | m-D5 | A-23 | m-D5 | H | H | H |
| 6792 | 0 | m-D5 | A-24 | m-D5 | H | H | H |
| 6793 | 0 | m-D5 | A-25 | m-D5 | H | H | H |
| 6794 | 0 | m-D5 | A-26 | m-D5 | H | H | H |
| 6795 | 0 | m-D5 | A-27 | m-D5 | H | H | H |
| 6796 | 0 | m-D5 | A-28 | m-D5 | H | H | H |
| 6797 | 0 | m-D5 | A-29 | m-D5 | H | H | H |
| 6798 | 0 | m-D5 | A-30 | m-D5 | H | H | H |
| 6799 | 0 | m-D5 | A-31 | m-D5 | H | H | H |
| 6800 | 0 | m-D5 | A-32 | m-D5 | H | H | H |
| 6801 | 0 | m-D5 | A-33 | m-D5 | H | H | H |
| 6802 | 0 | m-D5 | A-34 | m-D5 | H | H | H |
| 6803 | 0 | m-D5 | A-35 | m-D5 | H | H | H |
| 6804 | 0 | m-D5 | A-36 | m-D5 | H | H | H |
| 6805 | 0 | m-D5 | A-37 | m-D5 | H | H | H |
| 6806 | 0 | m-D5 | A-38 | m-D5 | H | H | H |
| 6807 | 0 | m-D5 | A-39 | m-D5 | H | H | H |
| 6808 | 0 | m-D5 | A-40 | m-D5 | H | H | H |
| 6809 | 0 | m-D5 | A-41 | m-D5 | H | H | H |
| 6810 | 0 | m-D5 | A-42 | m-D5 | H | H | H |
| 6811 | 0 | m-D5 | A-43 | m-D5 | H | H | H |
| 6812 | 0 | m-D5 | A-44 | m-D5 | H | H | H |
| 6813 | 0 | m-D5 | A-45 | m-D5 | H | H | H |
| 6814 | 0 | m-D5 | A-46 | m-D5 | H | H | H |
| 6815 | 0 | m-D5 | A-47 | m-D5 | H | H | H |
| 6816 | 0 | m-D9 | A-1 | m-D9 | H | H | H |
| 6817 | 0 | m-D9 | A-2 | m-D9 | H | H | H |
| 6818 | 0 | m-D9 | A-3 | m-D9 | H | H | H |
| 6819 | 0 | m-D9 | A-4 | m-D9 | H | H | H |
| 6820 | 0 | m-D9 | A-5 | m-D9 | H | H | H |
| 6821 | 0 | m-D9 | A-6 | m-D9 | H | H | H |
| 6822 | 0 | m-D9 | A-7 | m-D9 | H | H | H |
| 6823 | 0 | m-D9 | A-8 | m-D9 | H | H | H |
| 6824 | 0 | m-D9 | A-9 | m-D9 | H | H | H |
| 6825 | 0 | m-D9 | A-10 | m-D9 | H | H | H |
| 6826 | 0 | m-D9 | A-11 | m-D9 | H | H | H |
| 6827 | 0 | m-D9 | A-12 | m-D9 | H | H | H |
| 6828 | 0 | m-D9 | A-13 | m-D9 | H | H | H |
| 6829 | 0 | m-D9 | A-14 | m-D9 | H | H | H |
| 6830 | 0 | m-D9 | A-15 | m-D9 | H | H | H |
| 6831 | 0 | m-D9 | A-16 | m-D9 | H | H | H |
| 6832 | 0 | m-D9 | A-17 | m-D9 | H | H | H |
| 6833 | 0 | m-D9 | A-18 | m-D9 | H | H | H |
| 6834 | 0 | m-D9 | A-19 | m-D9 | H | H | H |
| 6835 | 0 | m-D9 | A-20 | m-D9 | H | H | H |
| 6836 | 0 | m-D9 | A-21 | m-D9 | H | H | H |
| 6837 | 0 | m-D9 | A-22 | m-D9 | H | H | H |
| 6838 | 0 | m-D9 | A-23 | m-D9 | H | H | H |
| 6839 | 0 | m-D9 | A-24 | m-D9 | H | H | H |
| 6840 | 0 | m-D9 | A-25 | m-D9 | H | H | H |
| 6841 | 0 | m-D9 | A-26 | m-D9 | H | H | H |
| 6842 | 0 | m-D9 | A-27 | m-D9 | H | H | H |
| 6843 | 0 | m-D9 | A-28 | m-D9 | H | H | H |
| 6844 | 0 | m-D9 | A-29 | m-D9 | H | H | H |
| 6845 | 0 | m-D9 | A-30 | m-D9 | H | H | H |
| 6846 | 0 | m-D9 | A-31 | m-D9 | H | H | H |
| 6847 | 0 | m-D9 | A-32 | m-D9 | H | H | H |
| 6848 | 0 | m-D9 | A-33 | m-D9 | H | H | H |
| 6849 | 0 | m-D9 | A-34 | m-D9 | H | H | H |
| 6850 | 0 | m-D9 | A-35 | m-D9 | H | H | H |
| 6851 | 0 | m-D9 | A-36 | m-D9 | H | H | H |
| 6852 | 0 | m-D9 | A-37 | m-D9 | H | H | H |
| 6853 | 0 | m-D9 | A-38 | m-D9 | H | H | H |
| 6854 | 0 | m-D9 | A-39 | m-D9 | H | H | H |
| 6855 | 0 | m-D9 | A-40 | m-D9 | H | H | H |
| 6856 | 0 | m-D9 | A-41 | m-D9 | H | H | H |
| 6857 | 0 | m-D9 | A-42 | m-D9 | H | H | H |
| 6858 | 0 | m-D9 | A-43 | m-D9 | H | H | H |
| 6859 | 0 | m-D9 | A-44 | m-D9 | H | H | H |
| 6860 | 0 | m-D9 | A-45 | m-D9 | H | H | H |
| 6861 | 0 | m-D9 | A-46 | m-D9 | H | H | H |
| 6862 | 0 | m-D9 | A-47 | m-D9 | H | H | H |
| 6863 | 0 | m-D11 | A-1 | m-D11 | H | H | H |
| 6864 | 0 | m-D11 | A-2 | m-D11 | H | H | H |
| 6865 | 0 | m-D11 | A-3 | m-D11 | H | H | H |
| 6866 | 0 | m-D11 | A-4 | m-D11 | H | H | H |
| 6867 | 0 | m-D11 | A-5 | m-D11 | H | H | H |
| 6868 | 0 | m-D11 | A-6 | m-D11 | H | H | H |
| 6869 | 0 | m-D11 | A-7 | m-D11 | H | H | H |
| 6870 | 0 | m-D11 | A-8 | m-D11 | H | H | H |
| 6871 | 0 | m-D11 | A-9 | m-D11 | H | H | H |
| 6872 | 0 | m-D11 | A-10 | m-D11 | H | H | H |
| 6873 | 0 | m-D11 | A-11 | m-D11 | H | H | H |
| 6874 | 0 | m-D11 | A-12 | m-D11 | H | H | H |
| 6875 | 0 | m-D11 | A-13 | m-D11 | H | H | H |
| 6876 | 0 | m-D11 | A-14 | m-D11 | H | H | H |
| 6877 | 0 | m-D11 | A-15 | m-D11 | H | H | H |
| 6878 | 0 | m-D11 | A-16 | m-D11 | H | H | H |
| 6879 | 0 | m-D11 | A-17 | m-D11 | H | H | H |
| 6880 | 0 | m-D11 | A-18 | m-D11 | H | H | H |
| 6881 | 0 | m-D11 | A-19 | m-D11 | H | H | H |
| 6882 | 0 | m-D11 | A-20 | m-D11 | H | H | H |
| 6883 | 0 | m-D11 | A-21 | m-D11 | H | H | H |
| 6884 | 0 | m-D11 | A-22 | m-D11 | H | H | H |
| 6885 | 0 | m-D11 | A-23 | m-D11 | H | H | H |
| 6886 | 0 | m-D11 | A-24 | m-D11 | H | H | H |
| 6887 | 0 | m-D11 | A-25 | m-D11 | H | H | H |
| 6888 | 0 | m-D11 | A-26 | m-D11 | H | H | H |
| 6889 | 0 | m-D11 | A-27 | m-D11 | H | H | H |
| 6890 | 0 | m-D11 | A-28 | m-D11 | H | H | H |
| 6891 | 0 | m-D11 | A-29 | m-D11 | H | H | H |
| 6892 | 0 | m-D11 | A-30 | m-D11 | H | H | H |
| 6893 | 0 | m-D11 | A-31 | m-D11 | H | H | H |
| 6894 | 0 | m-D11 | A-32 | m-D11 | H | H | H |
| 6895 | 0 | m-D11 | A-33 | m-D11 | H | H | H |
| 6896 | 0 | m-D11 | A-34 | m-D11 | H | H | H |
| 6897 | 0 | m-D11 | A-35 | m-D11 | H | H | H |
| 6898 | 0 | m-D11 | A-36 | m-D11 | H | H | H |
| 6899 | 0 | m-D11 | A-37 | m-D11 | H | H | H |
| 6900 | 0 | m-D11 | A-38 | m-D11 | H | H | H |
| 6901 | 0 | m-D11 | A-39 | m-D11 | H | H | H |
| 6902 | 0 | m-D11 | A-40 | m-D11 | H | H | H |
| 6903 | 0 | m-D11 | A-41 | m-D11 | H | H | H |
| 6904 | 0 | m-D11 | A-42 | m-D11 | H | H | H |
| 6905 | 0 | m-D11 | A-43 | m-D11 | H | H | H |
| 6906 | 0 | m-D11 | A-44 | m-D11 | H | H | H |
| 6907 | 0 | m-D11 | A-45 | m-D11 | H | H | H |
| 6908 | 0 | m-D11 | A-46 | m-D11 | H | H | H |
| 6909 | 0 | m-D11 | A-47 | m-D11 | H | H | H |
| 6910 | 0 | m-D12 | A-1 | m-D12 | H | H | H |
| 6911 | 0 | m-D12 | A-2 | m-D12 | H | H | H |
| 6912 | 0 | m-D12 | A-3 | m-D12 | H | H | H |
| 6913 | 0 | m-D12 | A-4 | m-D12 | H | H | H |
| 6914 | 0 | m-D12 | A-5 | m-D12 | H | H | H |
| 6915 | 0 | m-D12 | A-6 | m-D12 | H | H | H |
| 6916 | 0 | m-D12 | A-7 | m-D12 | H | H | H |
| 6917 | 0 | m-D12 | A-8 | m-D12 | H | H | H |
| 6918 | 0 | m-D12 | A-9 | m-D12 | H | H | H |
| 6919 | 0 | m-D12 | A-10 | m-D12 | H | H | H |
| 6920 | 0 | m-D12 | A-11 | m-D12 | H | H | H |
| 6921 | 0 | m-D12 | A-12 | m-D12 | H | H | H |
| 6922 | 0 | m-D12 | A-13 | m-D12 | H | H | H |
| 6923 | 0 | m-D12 | A-14 | m-D12 | H | H | H |
| 6924 | 0 | m-D12 | A-15 | m-D12 | H | H | H |
| 6925 | 0 | m-D12 | A-16 | m-D12 | H | H | H |
| 6926 | 0 | m-D12 | A-17 | m-D12 | H | H | H |
| 6927 | 0 | m-D12 | A-18 | m-D12 | H | H | H |
| 6928 | 0 | m-D12 | A-19 | m-D12 | H | H | H |
| 6929 | 0 | m-D12 | A-20 | m-D12 | H | H | H |
| 6930 | 0 | m-D12 | A-21 | m-D12 | H | H | H |
| 6931 | 0 | m-D12 | A-22 | m-D12 | H | H | H |
| 6932 | 0 | m-D12 | A-23 | m-D12 | H | H | H |
| 6933 | 0 | m-D12 | A-24 | m-D12 | H | H | H |
| 6934 | 0 | m-D12 | A-25 | m-D12 | H | H | H |
| 6935 | 0 | m-D12 | A-26 | m-D12 | H | H | H |
| 6936 | 0 | m-D12 | A-27 | m-D12 | H | H | H |
| 6937 | 0 | m-D12 | A-28 | m-D12 | H | H | H |
| 6938 | 0 | m-D12 | A-29 | m-D12 | H | H | H |
| 6939 | 0 | m-D12 | A-30 | m-D12 | H | H | H |
| 6940 | 0 | m-D12 | A-31 | m-D12 | H | H | H |
| 6941 | 0 | m-D12 | A-32 | m-D12 | H | H | H |
| 6942 | 0 | m-D12 | A-33 | m-D12 | H | H | H |
| 6943 | 0 | m-D12 | A-34 | m-D12 | H | H | H |
| 6944 | 0 | m-D12 | A-35 | m-D12 | H | H | H |
| 6945 | 0 | m-D12 | A-36 | m-D12 | H | H | H |
| 6946 | 0 | m-D12 | A-37 | m-D12 | H | H | H |
| 6947 | 0 | m-D12 | A-38 | m-D12 | H | H | H |
| 6948 | 0 | m-D12 | A-39 | m-D12 | H | H | H |
| 6949 | 0 | m-D12 | A-40 | m-D12 | H | H | H |
| 6950 | 0 | m-D12 | A-41 | m-D12 | H | H | H |
| 6951 | 0 | m-D12 | A-42 | m-D12 | H | H | H |
| 6952 | 0 | m-D12 | A-43 | m-D12 | H | H | H |
| 6953 | 0 | m-D12 | A-44 | m-D12 | H | H | H |
| 6954 | 0 | m-D12 | A-45 | m-D12 | H | H | H |
| 6955 | 0 | m-D12 | A-46 | m-D12 | H | H | H |
| 6956 | 0 | m-D12 | A-47 | m-D12 | H | H | H |
| 6957 | 0 | m-D13 | A-1 | m-D13 | H | H | H |
| 6958 | 0 | m-D13 | A-2 | m-D13 | H | H | H |
| 6959 | 0 | m-D13 | A-3 | m-D13 | H | H | H |
| 6960 | 0 | m-D13 | A-4 | m-D13 | H | H | H |
| 6961 | 0 | m-D13 | A-5 | m-D13 | H | H | H |
| 6962 | 0 | m-D13 | A-6 | m-D13 | H | H | H |
| 6963 | 0 | m-D13 | A-7 | m-D13 | H | H | H |
| 6964 | 0 | m-D13 | A-8 | m-D13 | H | H | H |
| 6965 | 0 | m-D13 | A-9 | m-D13 | H | H | H |
| 6966 | 0 | m-D13 | A-10 | m-D13 | H | H | H |
| 6967 | 0 | m-D13 | A-11 | m-D13 | H | H | H |
| 6968 | 0 | m-D13 | A-12 | m-D13 | H | H | H |
| 6969 | 0 | m-D13 | A-13 | m-D13 | H | H | H |
| 6970 | 0 | m-D13 | A-14 | m-D13 | H | H | H |
| 6971 | 0 | m-D13 | A-15 | m-D13 | H | H | H |
| 6972 | 0 | m-D13 | A-16 | m-D13 | H | H | H |
| 6973 | 0 | m-D13 | A-17 | m-D13 | H | H | H |
| 6974 | 0 | m-D13 | A-18 | m-D13 | H | H | H |
| 6975 | 0 | m-D13 | A-19 | m-D13 | H | H | H |
| 6976 | 0 | m-D13 | A-20 | m-D13 | H | H | H |
| 6977 | 0 | m-D13 | A-21 | m-D13 | H | H | H |
| 6978 | 0 | m-D13 | A-22 | m-D13 | H | H | H |
| 6979 | 0 | m-D13 | A-23 | m-D13 | H | H | H |
| 6980 | 0 | m-D13 | A-24 | m-D13 | H | H | H |
| 6981 | 0 | m-D13 | A-25 | m-D13 | H | H | H |
| 6982 | 0 | m-D13 | A-26 | m-D13 | H | H | H |
| 6983 | 0 | m-D13 | A-27 | m-D13 | H | H | H |
| 6984 | 0 | m-D13 | A-28 | m-D13 | H | H | H |
| 6985 | 0 | m-D13 | A-29 | m-D13 | H | H | H |
| 6986 | 0 | m-D13 | A-30 | m-D13 | H | H | H |
| 6987 | 0 | m-D13 | A-31 | m-D13 | H | H | H |
| 6988 | 0 | m-D13 | A-32 | m-D13 | H | H | H |
| 6989 | 0 | m-D13 | A-33 | m-D13 | H | H | H |
| 6990 | 0 | m-D13 | A-34 | m-D13 | H | H | H |
| 6991 | 0 | m-D13 | A-35 | m-D13 | H | H | H |
| 6992 | 0 | m-D13 | A-36 | m-D13 | H | H | H |
| 6993 | 0 | m-D13 | A-37 | m-D13 | H | H | H |
| 6994 | 0 | m-D13 | A-38 | m-D13 | H | H | H |
| 6995 | 0 | m-D13 | A-39 | m-D13 | H | H | H |
| 6996 | 0 | m-D13 | A-40 | m-D13 | H | H | H |
| 6997 | 0 | m-D13 | A-41 | m-D13 | H | H | H |
| 6998 | 0 | m-D13 | A-42 | m-D13 | H | H | H |
| 6999 | 0 | m-D13 | A-43 | m-D13 | H | H | H |
| 7000 | 0 | m-D13 | A-44 | m-D13 | H | H | H |
| 7001 | 0 | m-D13 | A-45 | m-D13 | H | H | H |
| 7002 | 0 | m-D13 | A-46 | m-D13 | H | H | H |
| 7003 | 0 | m-D13 | A-47 | m-D13 | H | H | H |
| 7004 | 0 | m-D14 | A-1 | m-D14 | H | H | H |
| 7005 | 0 | m-D14 | A-2 | m-D14 | H | H | H |
| 7006 | 0 | m-D14 | A-3 | m-D14 | H | H | H |
| 7007 | 0 | m-D14 | A-4 | m-D14 | H | H | H |
| 7008 | 0 | m-D14 | A-5 | m-D14 | H | H | H |
| 7009 | 0 | m-D14 | A-6 | m-D14 | H | H | H |
| 7010 | 0 | m-D14 | A-7 | m-D14 | H | H | H |
| 7011 | 0 | m-D14 | A-8 | m-D14 | H | H | H |
| 7012 | 0 | m-D14 | A-9 | m-D14 | H | H | H |
| 7013 | 0 | m-D14 | A-10 | m-D14 | H | H | H |
| 7014 | 0 | m-D14 | A-11 | m-D14 | H | H | H |
| 7015 | 0 | m-D14 | A-12 | m-D14 | H | H | H |
| 7016 | 0 | m-D14 | A-13 | m-D14 | H | H | H |
| 7017 | 0 | m-D14 | A-14 | m-D14 | H | H | H |
| 7018 | 0 | m-D14 | A-15 | m-D14 | H | H | H |
| 7019 | 0 | m-D14 | A-16 | m-D14 | H | H | H |
| 7020 | 0 | m-D14 | A-17 | m-D14 | H | H | H |
| 7021 | 0 | m-D14 | A-18 | m-D14 | H | H | H |
| 7022 | 0 | m-D14 | A-19 | m-D14 | H | H | H |
| 7023 | 0 | m-D14 | A-20 | m-D14 | H | H | H |
| 7024 | 0 | m-D14 | A-21 | m-D14 | H | H | H |
| 7025 | 0 | m-D14 | A-22 | m-D14 | H | H | H |
| 7026 | 0 | m-D14 | A-23 | m-D14 | H | H | H |
| 7027 | 0 | m-D14 | A-24 | m-D14 | H | H | H |
| 7028 | 0 | m-D14 | A-25 | m-D14 | H | H | H |
| 7029 | 0 | m-D14 | A-26 | m-D14 | H | H | H |
| 7030 | 0 | m-D14 | A-27 | m-D14 | H | H | H |
| 7031 | 0 | m-D14 | A-28 | m-D14 | H | H | H |
| 7032 | 0 | m-D14 | A-29 | m-D14 | H | H | H |
| 7033 | 0 | m-D14 | A-30 | m-D14 | H | H | H |
| 7034 | 0 | m-D14 | A-31 | m-D14 | H | H | H |
| 7035 | 0 | m-D14 | A-32 | m-D14 | H | H | H |
| 7036 | 0 | m-D14 | A-33 | m-D14 | H | H | H |
| 7037 | 0 | m-D14 | A-34 | m-D14 | H | H | H |
| 7038 | 0 | m-D14 | A-35 | m-D14 | H | H | H |
| 7039 | 0 | m-D14 | A-36 | m-D14 | H | H | H |
| 7040 | 0 | m-D14 | A-37 | m-D14 | H | H | H |
| 7041 | 0 | m-D14 | A-38 | m-D14 | H | H | H |
| 7042 | 0 | m-D14 | A-39 | m-D14 | H | H | H |
| 7043 | 0 | m-D14 | A-40 | m-D14 | H | H | H |
| 7044 | 0 | m-D14 | A-41 | m-D14 | H | H | H |
| 7045 | 0 | m-D14 | A-42 | m-D14 | H | H | H |
| 7046 | 0 | m-D14 | A-43 | m-D14 | H | H | H |
| 7047 | 0 | m-D14 | A-44 | m-D14 | H | H | H |
| 7048 | 0 | m-D14 | A-45 | m-D14 | H | H | H |
| 7049 | 0 | m-D14 | A-46 | m-D14 | H | H | H |
| 7050 | 0 | m-D14 | A-47 | m-D14 | H | H | H |
| 7051 | 0 | m-D1 | H | A-1 | A-1 | A-1 | H |
| 7052 | 0 | m-D1 | H | A-2 | A-2 | A-2 | H |
| 7053 | 0 | m-D1 | H | A-3 | A-3 | A-3 | H |
| 7054 | 0 | m-D1 | H | A-4 | A-4 | A-4 | H |
| 7055 | 0 | m-D1 | H | A-5 | A-5 | A-5 | H |
| 7056 | 0 | m-D1 | H | A-6 | A-6 | A-6 | H |
| 7057 | 0 | m-D1 | H | A-7 | A-7 | A-7 | H |
| 7058 | 0 | m-D1 | H | A-8 | A-8 | A-8 | H |
| 7059 | 0 | m-D1 | H | A-9 | A-9 | A-9 | H |
| 7060 | 0 | m-D1 | H | A-10 | A-10 | A-10 | H |
| 7061 | 0 | m-D1 | H | A-11 | A-11 | A-11 | H |
| 7062 | 0 | m-D1 | H | A-12 | A-12 | A-12 | H |
| 7063 | 0 | m-D1 | H | A-13 | A-13 | A-13 | H |
| 7064 | 0 | m-D1 | H | A-14 | A-14 | A-14 | H |
| 7065 | 0 | m-D1 | H | A-15 | A-15 | A-15 | H |
| 7066 | 0 | m-D1 | H | A-16 | A-16 | A-16 | H |
| 7067 | 0 | m-D1 | H | A-17 | A-17 | A-17 | H |
| 7068 | 0 | m-D1 | H | A-18 | A-18 | A-18 | H |
| 7069 | 0 | m-D1 | H | A-19 | A-19 | A-19 | H |
| 7070 | 0 | m-D1 | H | A-20 | A-20 | A-20 | H |
| 7071 | 0 | m-D1 | H | A-21 | A-21 | A-21 | H |
| 7072 | 0 | m-D1 | H | A-22 | A-22 | A-22 | H |
| 7073 | 0 | m-D1 | H | A-23 | A-23 | A-23 | H |
| 7074 | 0 | m-D1 | H | A-24 | A-24 | A-24 | H |
| 7075 | 0 | m-D1 | H | A-25 | A-25 | A-25 | H |
| 7076 | 0 | m-D1 | H | A-26 | A-26 | A-26 | H |
| 7077 | 0 | m-D1 | H | A-27 | A-27 | A-27 | H |
| 7078 | 0 | m-D1 | H | A-28 | A-28 | A-28 | H |
| 7079 | 0 | m-D1 | H | A-29 | A-29 | A-29 | H |
| 7080 | 0 | m-D1 | H | A-30 | A-30 | A-30 | H |
| 7081 | 0 | m-D1 | H | A-31 | A-31 | A-31 | H |
| 7082 | 0 | m-D1 | H | A-32 | A-32 | A-32 | H |
| 7083 | 0 | m-D1 | H | A-33 | A-33 | A-33 | H |
| 7084 | 0 | m-D1 | H | A-34 | A-34 | A-34 | H |
| 7085 | 0 | m-D1 | H | A-35 | A-35 | A-35 | H |
| 7086 | 0 | m-D1 | H | A-36 | A-36 | A-36 | H |
| 7087 | 0 | m-D1 | H | A-37 | A-37 | A-37 | H |
| 7088 | 0 | m-D1 | H | A-38 | A-38 | A-38 | H |
| 7089 | 0 | m-D1 | H | A-39 | A-39 | A-39 | H |
| 7090 | 0 | m-D1 | H | A-40 | A-40 | A-40 | H |
| 7091 | 0 | m-D1 | H | A-41 | A-41 | A-41 | H |
| 7092 | 0 | m-D1 | H | A-42 | A-42 | A-42 | H |
| 7093 | 0 | m-D1 | H | A-43 | A-43 | A-43 | H |
| 7094 | 0 | m-D1 | H | A-44 | A-44 | A-44 | H |
| 7095 | 0 | m-D1 | H | A-45 | A-45 | A-45 | H |
| 7096 | 0 | m-D1 | H | A-46 | A-46 | A-46 | H |
| 7097 | 0 | m-D1 | H | A-47 | A-47 | A-47 | H |
| 7098 | 0 | m-D2 | H | A-1 | A-1 | A-1 | H |
| 7099 | 0 | m-D2 | H | A-2 | A-2 | A-2 | H |
| 7100 | 0 | m-D2 | H | A-3 | A-3 | A-3 | H |
| 7101 | 0 | m-D2 | H | A-4 | A-4 | A-4 | H |
| 7102 | 0 | m-D2 | H | A-5 | A-5 | A-5 | H |
| 7103 | 0 | m-D2 | H | A-6 | A-6 | A-6 | H |
| 7104 | 0 | m-D2 | H | A-7 | A-7 | A-7 | H |
| 7105 | 0 | m-D2 | H | A-8 | A-8 | A-8 | H |
| 7106 | 0 | m-D2 | H | A-9 | A-9 | A-9 | H |
| 7107 | 0 | m-D2 | H | A-10 | A-10 | A-10 | H |
| 7108 | 0 | m-D2 | H | A-11 | A-11 | A-11 | H |
| 7109 | 0 | m-D2 | H | A-12 | A-12 | A-12 | H |
| 7110 | 0 | m-D2 | H | A-13 | A-13 | A-13 | H |
| 7111 | 0 | m-D2 | H | A-14 | A-14 | A-14 | H |
| 7112 | 0 | m-D2 | H | A-15 | A-15 | A-15 | H |
| 7113 | 0 | m-D2 | H | A-16 | A-16 | A-16 | H |
| 7114 | 0 | m-D2 | H | A-17 | A-17 | A-17 | H |
| 7115 | 0 | m-D2 | H | A-18 | A-18 | A-18 | H |
| 7116 | 0 | m-D2 | H | A-19 | A-19 | A-19 | H |
| 7117 | 0 | m-D2 | H | A-20 | A-20 | A-20 | H |
| 7118 | 0 | m-D2 | H | A-21 | A-21 | A-21 | H |
| 7119 | 0 | m-D2 | H | A-22 | A-22 | A-22 | H |
| 7120 | 0 | m-D2 | H | A-23 | A-23 | A-23 | H |
| 7121 | 0 | m-D2 | H | A-24 | A-24 | A-24 | H |
| 7122 | 0 | m-D2 | H | A-25 | A-25 | A-25 | H |
| 7123 | 0 | m-D2 | H | A-26 | A-26 | A-26 | H |
| 7124 | 0 | m-D2 | H | A-27 | A-27 | A-27 | H |
| 7125 | 0 | m-D2 | H | A-28 | A-28 | A-28 | H |
| 7126 | 0 | m-D2 | H | A-29 | A-29 | A-29 | H |
| 7127 | 0 | m-D2 | H | A-30 | A-30 | A-30 | H |
| 7128 | 0 | m-D2 | H | A-31 | A-31 | A-31 | H |
| 7129 | 0 | m-D2 | H | A-32 | A-32 | A-32 | H |
| 7130 | 0 | m-D2 | H | A-33 | A-33 | A-33 | H |
| 7131 | 0 | m-D2 | H | A-34 | A-34 | A-34 | H |
| 7132 | 0 | m-D2 | H | A-35 | A-35 | A-35 | H |
| 7133 | 0 | m-D2 | H | A-36 | A-36 | A-36 | H |
| 7134 | 0 | m-D2 | H | A-37 | A-37 | A-37 | H |
| 7135 | 0 | m-D2 | H | A-38 | A-38 | A-38 | H |
| 7136 | 0 | m-D2 | H | A-39 | A-39 | A-39 | H |
| 7137 | 0 | m-D2 | H | A-40 | A-40 | A-40 | H |
| 7138 | 0 | m-D2 | H | A-41 | A-41 | A-41 | H |
| 7139 | 0 | m-D2 | H | A-42 | A-42 | A-42 | H |
| 7140 | 0 | m-D2 | H | A-43 | A-43 | A-43 | H |
| 7141 | 0 | m-D2 | H | A-44 | A-44 | A-44 | H |
| 7142 | 0 | m-D2 | H | A-45 | A-45 | A-45 | H |
| 7143 | 0 | m-D2 | H | A-46 | A-46 | A-46 | H |
| 7144 | 0 | m-D2 | H | A-47 | A-47 | A-47 | H |
| 7145 | 0 | m-D3 | H | A-1 | A-1 | A-1 | H |
| 7146 | 0 | m-D3 | H | A-2 | A-2 | A-2 | H |
| 7147 | 0 | m-D3 | H | A-3 | A-3 | A-3 | H |
| 7148 | 0 | m-D3 | H | A-4 | A-4 | A-4 | H |
| 7149 | 0 | m-D3 | H | A-5 | A-5 | A-5 | H |
| 7150 | 0 | m-D3 | H | A-6 | A-6 | A-6 | H |
| 7151 | 0 | m-D3 | H | A-7 | A-7 | A-7 | H |
| 7152 | 0 | m-D3 | H | A-8 | A-8 | A-8 | H |
| 7153 | 0 | m-D3 | H | A-9 | A-9 | A-9 | H |
| 7154 | 0 | m-D3 | H | A-10 | A-10 | A-10 | H |
| 7155 | 0 | m-D3 | H | A-11 | A-11 | A-11 | H |
| 7156 | 0 | m-D3 | H | A-12 | A-12 | A-12 | H |
| 7157 | 0 | m-D3 | H | A-13 | A-13 | A-13 | H |
| 7158 | 0 | m-D3 | H | A-14 | A-14 | A-14 | H |
| 7159 | 0 | m-D3 | H | A-15 | A-15 | A-15 | H |
| 7160 | 0 | m-D3 | H | A-16 | A-16 | A-16 | H |
| 7161 | 0 | m-D3 | H | A-17 | A-17 | A-17 | H |
| 7162 | 0 | m-D3 | H | A-18 | A-18 | A-18 | H |
| 7163 | 0 | m-D3 | H | A-19 | A-19 | A-19 | H |
| 7164 | 0 | m-D3 | H | A-20 | A-20 | A-20 | H |
| 7165 | 0 | m-D3 | H | A-21 | A-21 | A-21 | H |
| 7166 | 0 | m-D3 | H | A-22 | A-22 | A-22 | H |
| 7167 | 0 | m-D3 | H | A-23 | A-23 | A-23 | H |
| 7168 | 0 | m-D3 | H | A-24 | A-24 | A-24 | H |
| 7169 | 0 | m-D3 | H | A-25 | A-25 | A-25 | H |
| 7170 | 0 | m-D3 | H | A-26 | A-26 | A-26 | H |
| 7171 | 0 | m-D3 | H | A-27 | A-27 | A-27 | H |
| 7172 | 0 | m-D3 | H | A-28 | A-28 | A-28 | H |
| 7173 | 0 | m-D3 | H | A-29 | A-29 | A-29 | H |
| 7174 | 0 | m-D3 | H | A-30 | A-30 | A-30 | H |
| 7175 | 0 | m-D3 | H | A-31 | A-31 | A-31 | H |
| 7176 | 0 | m-D3 | H | A-32 | A-32 | A-32 | H |
| 7177 | 0 | m-D3 | H | A-33 | A-33 | A-33 | H |
| 7178 | 0 | m-D3 | H | A-34 | A-34 | A-34 | H |
| 7179 | 0 | m-D3 | H | A-35 | A-35 | A-35 | H |
| 7180 | 0 | m-D3 | H | A-36 | A-36 | A-36 | H |
| 7181 | 0 | m-D3 | H | A-37 | A-37 | A-37 | H |
| 7182 | 0 | m-D3 | H | A-38 | A-38 | A-38 | H |
| 7183 | 0 | m-D3 | H | A-39 | A-39 | A-39 | H |
| 7184 | 0 | m-D3 | H | A-40 | A-40 | A-40 | H |
| 7185 | 0 | m-D3 | H | A-41 | A-41 | A-41 | H |
| 7186 | 0 | m-D3 | H | A-42 | A-42 | A-42 | H |
| 7187 | 0 | m-D3 | H | A-43 | A-43 | A-43 | H |
| 7188 | 0 | m-D3 | H | A-44 | A-44 | A-44 | H |
| 7189 | 0 | m-D3 | H | A-45 | A-45 | A-45 | H |
| 7190 | 0 | m-D3 | H | A-46 | A-46 | A-46 | H |
| 7191 | 0 | m-D3 | H | A-47 | A-47 | A-47 | H |
| 7192 | 0 | m-D5 | H | A-1 | A-1 | A-1 | H |
| 7193 | 0 | m-D5 | H | A-2 | A-2 | A-2 | H |
| 7194 | 0 | m-D5 | H | A-3 | A-3 | A-3 | H |
| 7195 | 0 | m-D5 | H | A-4 | A-4 | A-4 | H |
| 7196 | 0 | m-D5 | H | A-5 | A-5 | A-5 | H |
| 7197 | 0 | m-D5 | H | A-6 | A-6 | A-6 | H |
| 7198 | 0 | m-D5 | H | A-7 | A-7 | A-7 | H |
| 7199 | 0 | m-D5 | H | A-8 | A-8 | A-8 | H |
| 7200 | 0 | m-D5 | H | A-9 | A-9 | A-9 | H |
| 7201 | 0 | m-D5 | H | A-10 | A-10 | A-10 | H |
| 7202 | 0 | m-D5 | H | A-11 | A-11 | A-11 | H |
| 7203 | 0 | m-D5 | H | A-12 | A-12 | A-12 | H |
| 7204 | 0 | m-D5 | H | A-13 | A-13 | A-13 | H |
| 7205 | 0 | m-D5 | H | A-14 | A-14 | A-14 | H |
| 7206 | 0 | m-D5 | H | A-15 | A-15 | A-15 | H |
| 7207 | 0 | m-D5 | H | A-16 | A-16 | A-16 | H |
| 7208 | 0 | m-D5 | H | A-17 | A-17 | A-17 | H |
| 7209 | 0 | m-D5 | H | A-18 | A-18 | A-18 | H |
| 7210 | 0 | m-D5 | H | A-19 | A-19 | A-19 | H |
| 7211 | 0 | m-D5 | H | A-20 | A-20 | A-20 | H |
| 7212 | 0 | m-D5 | H | A-21 | A-21 | A-21 | H |
| 7213 | 0 | m-D5 | H | A-22 | A-22 | A-22 | H |
| 7214 | 0 | m-D5 | H | A-23 | A-23 | A-23 | H |
| 7215 | 0 | m-D5 | H | A-24 | A-24 | A-24 | H |
| 7216 | 0 | m-D5 | H | A-25 | A-25 | A-25 | H |
| 7217 | 0 | m-D5 | H | A-26 | A-26 | A-26 | H |
| 7218 | 0 | m-D5 | H | A-27 | A-27 | A-27 | H |
| 7219 | 0 | m-D5 | H | A-28 | A-28 | A-28 | H |
| 7220 | 0 | m-D5 | H | A-29 | A-29 | A-29 | H |
| 7221 | 0 | m-D5 | H | A-30 | A-30 | A-30 | H |
| 7222 | 0 | m-D5 | H | A-31 | A-31 | A-31 | H |
| 7223 | 0 | m-D5 | H | A-32 | A-32 | A-32 | H |
| 7224 | 0 | m-D5 | H | A-33 | A-33 | A-33 | H |
| 7225 | 0 | m-D5 | H | A-34 | A-34 | A-34 | H |
| 7226 | 0 | m-D5 | H | A-35 | A-35 | A-35 | H |
| 7227 | 0 | m-D5 | H | A-36 | A-36 | A-36 | H |
| 7228 | 0 | m-D5 | H | A-37 | A-37 | A-37 | H |
| 7229 | 0 | m-D5 | H | A-38 | A-38 | A-38 | H |
| 7230 | 0 | m-D5 | H | A-39 | A-39 | A-39 | H |
| 7231 | 0 | m-D5 | H | A-40 | A-40 | A-40 | H |
| 7232 | 0 | m-D5 | H | A-41 | A-41 | A-41 | H |
| 7233 | 0 | m-D5 | H | A-42 | A-42 | A-42 | H |
| 7234 | 0 | m-D5 | H | A-43 | A-43 | A-43 | H |
| 7235 | 0 | m-D5 | H | A-44 | A-44 | A-44 | H |
| 7236 | 0 | m-D5 | H | A-45 | A-45 | A-45 | H |
| 7237 | 0 | m-D5 | H | A-46 | A-46 | A-46 | H |
| 7238 | 0 | m-D5 | H | A-47 | A-47 | A-47 | H |
| 7239 | 0 | m-D9 | H | A-1 | A-1 | A-1 | H |
| 7240 | 0 | m-D9 | H | A-2 | A-2 | A-2 | H |
| 7241 | 0 | m-D9 | H | A-3 | A-3 | A-3 | H |
| 7242 | 0 | m-D9 | H | A-4 | A-4 | A-4 | H |
| 7243 | 0 | m-D9 | H | A-5 | A-5 | A-5 | H |
| 7244 | 0 | m-D9 | H | A-6 | A-6 | A-6 | H |
| 7245 | 0 | m-D9 | H | A-7 | A-7 | A-7 | H |
| 7246 | 0 | m-D9 | H | A-8 | A-8 | A-8 | H |
| 7247 | 0 | m-D9 | H | A-9 | A-9 | A-9 | H |
| 7248 | 0 | m-D9 | H | A-10 | A-10 | A-10 | H |
| 7249 | 0 | m-D9 | H | A-11 | A-11 | A-11 | H |
| 7250 | 0 | m-D9 | H | A-12 | A-12 | A-12 | H |
| 7251 | 0 | m-D9 | H | A-13 | A-13 | A-13 | H |
| 7252 | 0 | m-D9 | H | A-14 | A-14 | A-14 | H |
| 7253 | 0 | m-D9 | H | A-15 | A-15 | A-15 | H |
| 7254 | 0 | m-D9 | H | A-16 | A-16 | A-16 | H |
| 7255 | 0 | m-D9 | H | A-17 | A-17 | A-17 | H |
| 7256 | 0 | m-D9 | H | A-18 | A-18 | A-18 | H |
| 7257 | 0 | m-D9 | H | A-19 | A-19 | A-19 | H |
| 7258 | 0 | m-D9 | H | A-20 | A-20 | A-20 | H |
| 7259 | 0 | m-D9 | H | A-21 | A-21 | A-21 | H |
| 7260 | 0 | m-D9 | H | A-22 | A-22 | A-22 | H |
| 7261 | 0 | m-D9 | H | A-23 | A-23 | A-23 | H |
| 7262 | 0 | m-D9 | H | A-24 | A-24 | A-24 | H |
| 7263 | 0 | m-D9 | H | A-25 | A-25 | A-25 | H |
| 7264 | 0 | m-D9 | H | A-26 | A-26 | A-26 | H |
| 7265 | 0 | m-D9 | H | A-27 | A-27 | A-27 | H |
| 7266 | 0 | m-D9 | H | A-28 | A-28 | A-28 | H |
| 7267 | 0 | m-D9 | H | A-29 | A-29 | A-29 | H |
| 7268 | 0 | m-D9 | H | A-30 | A-30 | A-30 | H |
| 7269 | 0 | m-D9 | H | A-31 | A-31 | A-31 | H |
| 7270 | 0 | m-D9 | H | A-32 | A-32 | A-32 | H |
| 7271 | 0 | m-D9 | H | A-33 | A-33 | A-33 | H |
| 7272 | 0 | m-D9 | H | A-34 | A-34 | A-34 | H |
| 7273 | 0 | m-D9 | H | A-35 | A-35 | A-35 | H |
| 7274 | 0 | m-D9 | H | A-36 | A-36 | A-36 | H |
| 7275 | 0 | m-D9 | H | A-37 | A-37 | A-37 | H |
| 7276 | 0 | m-D9 | H | A-38 | A-38 | A-38 | H |
| 7277 | 0 | m-D9 | H | A-39 | A-39 | A-39 | H |
| 7278 | 0 | m-D9 | H | A-40 | A-40 | A-40 | H |
| 7279 | 0 | m-D9 | H | A-41 | A-41 | A-41 | H |
| 7280 | 0 | m-D9 | H | A-42 | A-42 | A-42 | H |
| 7281 | 0 | m-D9 | H | A-43 | A-43 | A-43 | H |
| 7282 | 0 | m-D9 | H | A-44 | A-44 | A-44 | H |
| 7283 | 0 | m-D9 | H | A-45 | A-45 | A-45 | H |
| 7284 | 0 | m-D9 | H | A-46 | A-46 | A-46 | H |
| 7285 | 0 | m-D9 | H | A-47 | A-47 | A-47 | H |
| 7286 | 0 | m-D11 | H | A-1 | A-1 | A-1 | H |
| 7287 | 0 | m-D11 | H | A-2 | A-2 | A-2 | H |
| 7288 | 0 | m-D11 | H | A-3 | A-3 | A-3 | H |
| 7289 | 0 | m-D11 | H | A-4 | A-4 | A-4 | H |
| 7290 | 0 | m-D11 | H | A-5 | A-5 | A-5 | H |
| 7291 | 0 | m-D11 | H | A-6 | A-6 | A-6 | H |
| 7292 | 0 | m-D11 | H | A-7 | A-7 | A-7 | H |
| 7293 | 0 | m-D11 | H | A-8 | A-8 | A-8 | H |
| 7294 | 0 | m-D11 | H | A-9 | A-9 | A-9 | H |
| 7295 | 0 | m-D11 | H | A-10 | A-10 | A-10 | H |
| 7296 | 0 | m-D11 | H | A-11 | A-11 | A-11 | H |
| 7297 | 0 | m-D11 | H | A-12 | A-12 | A-12 | H |
| 7298 | 0 | m-D11 | H | A-13 | A-13 | A-13 | H |
| 7299 | 0 | m-D11 | H | A-14 | A-14 | A-14 | H |
| 7300 | 0 | m-D11 | H | A-15 | A-15 | A-15 | H |
| 7301 | 0 | m-D11 | H | A-16 | A-16 | A-16 | H |
| 7302 | 0 | m-D11 | H | A-17 | A-17 | A-17 | H |
| 7303 | 0 | m-D11 | H | A-18 | A-18 | A-18 | H |
| 7304 | 0 | m-D11 | H | A-19 | A-19 | A-19 | H |
| 7305 | 0 | m-D11 | H | A-20 | A-20 | A-20 | H |
| 7306 | 0 | m-D11 | H | A-21 | A-21 | A-21 | H |
| 7307 | 0 | m-D11 | H | A-22 | A-22 | A-22 | H |
| 7308 | 0 | m-D11 | H | A-23 | A-23 | A-23 | H |
| 7309 | 0 | m-D11 | H | A-24 | A-24 | A-24 | H |
| 7310 | 0 | m-D11 | H | A-25 | A-25 | A-25 | H |
| 7311 | 0 | m-D11 | H | A-26 | A-26 | A-26 | H |
| 7312 | 0 | m-D11 | H | A-27 | A-27 | A-27 | H |
| 7313 | 0 | m-D11 | H | A-28 | A-28 | A-28 | H |
| 7314 | 0 | m-D11 | H | A-29 | A-29 | A-29 | H |
| 7315 | 0 | m-D11 | H | A-30 | A-30 | A-30 | H |
| 7316 | 0 | m-D11 | H | A-31 | A-31 | A-31 | H |
| 7317 | 0 | m-D11 | H | A-32 | A-32 | A-32 | H |
| 7318 | 0 | m-D11 | H | A-33 | A-33 | A-33 | H |
| 7319 | 0 | m-D11 | H | A-34 | A-34 | A-34 | H |
| 7320 | 0 | m-D11 | H | A-35 | A-35 | A-35 | H |
| 7321 | 0 | m-D11 | H | A-36 | A-36 | A-36 | H |
| 7322 | 0 | m-D11 | H | A-37 | A-37 | A-37 | H |
| 7323 | 0 | m-D11 | H | A-38 | A-38 | A-38 | H |
| 7324 | 0 | m-D11 | H | A-39 | A-39 | A-39 | H |
| 7325 | 0 | m-D11 | H | A-40 | A-40 | A-40 | H |
| 7326 | 0 | m-D11 | H | A-41 | A-41 | A-41 | H |
| 7327 | 0 | m-D11 | H | A-42 | A-42 | A-42 | H |
| 7328 | 0 | m-D11 | H | A-43 | A-43 | A-43 | H |
| 7329 | 0 | m-D11 | H | A-44 | A-44 | A-44 | H |
| 7330 | 0 | m-D11 | H | A-45 | A-45 | A-45 | H |
| 7331 | 0 | m-D11 | H | A-46 | A-46 | A-46 | H |
| 7332 | 0 | m-D11 | H | A-47 | A-47 | A-47 | H |
| 7333 | 0 | m-D12 | H | A-1 | A-1 | A-1 | H |
| 7334 | 0 | m-D12 | H | A-2 | A-2 | A-2 | H |
| 7335 | 0 | m-D12 | H | A-3 | A-3 | A-3 | H |
| 7336 | 0 | m-D12 | H | A-4 | A-4 | A-4 | H |
| 7337 | 0 | m-D12 | H | A-5 | A-5 | A-5 | H |
| 7338 | 0 | m-D12 | H | A-6 | A-6 | A-6 | H |
| 7339 | 0 | m-D12 | H | A-7 | A-7 | A-7 | H |
| 7340 | 0 | m-D12 | H | A-8 | A-8 | A-8 | H |
| 7341 | 0 | m-D12 | H | A-9 | A-9 | A-9 | H |
| 7342 | 0 | m-D12 | H | A-10 | A-10 | A-10 | H |
| 7343 | 0 | m-D12 | H | A-11 | A-11 | A-11 | H |
| 7344 | 0 | m-D12 | H | A-12 | A-12 | A-12 | H |
| 7345 | 0 | m-D12 | H | A-13 | A-13 | A-13 | H |
| 7346 | 0 | m-D12 | H | A-14 | A-14 | A-14 | H |
| 7347 | 0 | m-D12 | H | A-15 | A-15 | A-15 | H |
| 7348 | 0 | m-D12 | H | A-16 | A-16 | A-16 | H |
| 7349 | 0 | m-D12 | H | A-17 | A-17 | A-17 | H |
| 7350 | 0 | m-D12 | H | A-18 | A-18 | A-18 | H |
| 7351 | 0 | m-D12 | H | A-19 | A-19 | A-19 | H |
| 7352 | 0 | m-D12 | H | A-20 | A-20 | A-20 | H |
| 7353 | 0 | m-D12 | H | A-21 | A-21 | A-21 | H |
| 7354 | 0 | m-D12 | H | A-22 | A-22 | A-22 | H |
| 7355 | 0 | m-D12 | H | A-23 | A-23 | A-23 | H |
| 7356 | 0 | m-D12 | H | A-24 | A-24 | A-24 | H |
| 7357 | 0 | m-D12 | H | A-25 | A-25 | A-25 | H |
| 7358 | 0 | m-D12 | H | A-26 | A-26 | A-26 | H |
| 7359 | 0 | m-D12 | H | A-27 | A-27 | A-27 | H |
| 7360 | 0 | m-D12 | H | A-28 | A-28 | A-28 | H |
| 7361 | 0 | m-D12 | H | A-29 | A-29 | A-29 | H |
| 7362 | 0 | m-D12 | H | A-30 | A-30 | A-30 | H |
| 7363 | 0 | m-D12 | H | A-31 | A-31 | A-31 | H |
| 7364 | 0 | m-D12 | H | A-32 | A-32 | A-32 | H |
| 7365 | 0 | m-D12 | H | A-33 | A-33 | A-33 | H |
| 7366 | 0 | m-D12 | H | A-34 | A-34 | A-34 | H |
| 7367 | 0 | m-D12 | H | A-35 | A-35 | A-35 | H |
| 7368 | 0 | m-D12 | H | A-36 | A-36 | A-36 | H |
| 7369 | 0 | m-D12 | H | A-37 | A-37 | A-37 | H |
| 7370 | 0 | m-D12 | H | A-38 | A-38 | A-38 | H |
| 7371 | 0 | m-D12 | H | A-39 | A-39 | A-39 | H |
| 7372 | 0 | m-D12 | H | A-40 | A-40 | A-40 | H |
| 7373 | 0 | m-D12 | H | A-41 | A-41 | A-41 | H |
| 7374 | 0 | m-D12 | H | A-42 | A-42 | A-42 | H |
| 7375 | 0 | m-D12 | H | A-43 | A-43 | A-43 | H |
| 7376 | 0 | m-D12 | H | A-44 | A-44 | A-44 | H |
| 7377 | 0 | m-D12 | H | A-45 | A-45 | A-45 | H |
| 7378 | 0 | m-D12 | H | A-46 | A-46 | A-46 | H |
| 7379 | 0 | m-D12 | H | A-47 | A-47 | A-47 | H |
| 7380 | 0 | m-D13 | H | A-1 | A-1 | A-1 | H |
| 7381 | 0 | m-D13 | H | A-2 | A-2 | A-2 | H |
| 7382 | 0 | m-D13 | H | A-3 | A-3 | A-3 | H |
| 7383 | 0 | m-D13 | H | A-4 | A-4 | A-4 | H |
| 7384 | 0 | m-D13 | H | A-5 | A-5 | A-5 | H |
| 7385 | 0 | m-D13 | H | A-6 | A-6 | A-6 | H |
| 7386 | 0 | m-D13 | H | A-7 | A-7 | A-7 | H |
| 7387 | 0 | m-D13 | H | A-8 | A-8 | A-8 | H |
| 7388 | 0 | m-D13 | H | A-9 | A-9 | A-9 | H |
| 7389 | 0 | m-D13 | H | A-10 | A-10 | A-10 | H |
| 7390 | 0 | m-D13 | H | A-11 | A-11 | A-11 | H |
| 7391 | 0 | m-D13 | H | A-12 | A-12 | A-12 | H |
| 7392 | 0 | m-D13 | H | A-13 | A-13 | A-13 | H |
| 7393 | 0 | m-D13 | H | A-14 | A-14 | A-14 | H |
| 7394 | 0 | m-D13 | H | A-15 | A-15 | A-15 | H |
| 7395 | 0 | m-D13 | H | A-16 | A-16 | A-16 | H |
| 7396 | 0 | m-D13 | H | A-17 | A-17 | A-17 | H |
| 7397 | 0 | m-D13 | H | A-18 | A-18 | A-18 | H |
| 7398 | 0 | m-D13 | H | A-19 | A-19 | A-19 | H |
| 7399 | 0 | m-D13 | H | A-20 | A-20 | A-20 | H |
| 7400 | 0 | m-D13 | H | A-21 | A-21 | A-21 | H |
| 7401 | 0 | m-D13 | H | A-22 | A-22 | A-22 | H |
| 7402 | 0 | m-D13 | H | A-23 | A-23 | A-23 | H |
| 7403 | 0 | m-D13 | H | A-24 | A-24 | A-24 | H |
| 7404 | 0 | m-D13 | H | A-25 | A-25 | A-25 | H |
| 7405 | 0 | m-D13 | H | A-26 | A-26 | A-26 | H |
| 7406 | 0 | m-D13 | H | A-27 | A-27 | A-27 | H |
| 7407 | 0 | m-D13 | H | A-28 | A-28 | A-28 | H |
| 7408 | 0 | m-D13 | H | A-29 | A-29 | A-29 | H |
| 7409 | 0 | m-D13 | H | A-30 | A-30 | A-30 | H |
| 7410 | 0 | m-D13 | H | A-31 | A-31 | A-31 | H |
| 7411 | 0 | m-D13 | H | A-32 | A-32 | A-32 | H |
| 7412 | 0 | m-D13 | H | A-33 | A-33 | A-33 | H |
| 7413 | 0 | m-D13 | H | A-34 | A-34 | A-34 | H |
| 7414 | 0 | m-D13 | H | A-35 | A-35 | A-35 | H |
| 7415 | 0 | m-D13 | H | A-36 | A-36 | A-36 | H |
| 7416 | 0 | m-D13 | H | A-37 | A-37 | A-37 | H |
| 7417 | 0 | m-D13 | H | A-38 | A-38 | A-38 | H |
| 7418 | 0 | m-D13 | H | A-39 | A-39 | A-39 | H |
| 7419 | 0 | m-D13 | H | A-40 | A-40 | A-40 | H |
| 7420 | 0 | m-D13 | H | A-41 | A-41 | A-41 | H |
| 7421 | 0 | m-D13 | H | A-42 | A-42 | A-42 | H |
| 7422 | 0 | m-D13 | H | A-43 | A-43 | A-43 | H |
| 7423 | 0 | m-D13 | H | A-44 | A-44 | A-44 | H |
| 7424 | 0 | m-D13 | H | A-45 | A-45 | A-45 | H |
| 7425 | 0 | m-D13 | H | A-46 | A-46 | A-46 | H |
| 7426 | 0 | m-D13 | H | A-47 | A-47 | A-47 | H |
| 7427 | 0 | m-D14 | H | A-1 | A-1 | A-1 | H |
| 7428 | 0 | m-D14 | H | A-2 | A-2 | A-2 | H |
| 7429 | 0 | m-D14 | H | A-3 | A-3 | A-3 | H |
| 7430 | 0 | m-D14 | H | A-4 | A-4 | A-4 | H |
| 7431 | 0 | m-D14 | H | A-5 | A-5 | A-5 | H |
| 7432 | 0 | m-D14 | H | A-6 | A-6 | A-6 | H |
| 7433 | 0 | m-D14 | H | A-7 | A-7 | A-7 | H |
| 7434 | 0 | m-D14 | H | A-8 | A-8 | A-8 | H |
| 7435 | 0 | m-D14 | H | A-9 | A-9 | A-9 | H |
| 7436 | 0 | m-D14 | H | A-10 | A-10 | A-10 | H |
| 7437 | 0 | m-D14 | H | A-11 | A-11 | A-11 | H |
| 7438 | 0 | m-D14 | H | A-12 | A-12 | A-12 | H |
| 7439 | 0 | m-D14 | H | A-13 | A-13 | A-13 | H |
| 7440 | 0 | m-D14 | H | A-14 | A-14 | A-14 | H |
| 7441 | 0 | m-D14 | H | A-15 | A-15 | A-15 | H |
| 7442 | 0 | m-D14 | H | A-16 | A-16 | A-16 | H |
| 7443 | 0 | m-D14 | H | A-17 | A-17 | A-17 | H |
| 7444 | 0 | m-D14 | H | A-18 | A-18 | A-18 | H |
| 7445 | 0 | m-D14 | H | A-19 | A-19 | A-19 | H |
| 7446 | 0 | m-D14 | H | A-20 | A-20 | A-20 | H |
| 7447 | 0 | m-D14 | H | A-21 | A-21 | A-21 | H |
| 7448 | 0 | m-D14 | H | A-22 | A-22 | A-22 | H |
| 7449 | 0 | m-D14 | H | A-23 | A-23 | A-23 | H |
| 7450 | 0 | m-D14 | H | A-24 | A-24 | A-24 | H |
| 7451 | 0 | m-D14 | H | A-25 | A-25 | A-25 | H |
| 7452 | 0 | m-D14 | H | A-26 | A-26 | A-26 | H |
| 7453 | 0 | m-D14 | H | A-27 | A-27 | A-27 | H |
| 7454 | 0 | m-D14 | H | A-28 | A-28 | A-28 | H |
| 7455 | 0 | m-D14 | H | A-29 | A-29 | A-29 | H |
| 7456 | 0 | m-D14 | H | A-30 | A-30 | A-30 | H |
| 7457 | 0 | m-D14 | H | A-31 | A-31 | A-31 | H |
| 7458 | 0 | m-D14 | H | A-32 | A-32 | A-32 | H |
| 7459 | 0 | m-D14 | H | A-33 | A-33 | A-33 | H |
| 7460 | 0 | m-D14 | H | A-34 | A-34 | A-34 | H |
| 7461 | 0 | m-D14 | H | A-35 | A-35 | A-35 | H |
| 7462 | 0 | m-D14 | H | A-36 | A-36 | A-36 | H |
| 7463 | 0 | m-D14 | H | A-37 | A-37 | A-37 | H |
| 7464 | 0 | m-D14 | H | A-38 | A-38 | A-38 | H |
| 7465 | 0 | m-D14 | H | A-39 | A-39 | A-39 | H |
| 7466 | 0 | m-D14 | H | A-40 | A-40 | A-40 | H |
| 7467 | 0 | m-D14 | H | A-41 | A-41 | A-41 | H |
| 7468 | 0 | m-D14 | H | A-42 | A-42 | A-42 | H |
| 7469 | 0 | m-D14 | H | A-43 | A-43 | A-43 | H |
| 7470 | 0 | m-D14 | H | A-44 | A-44 | A-44 | H |
| 7471 | 0 | m-D14 | H | A-45 | A-45 | A-45 | H |
| 7472 | 0 | m-D14 | H | A-46 | A-46 | A-46 | H |
| 7473 | 0 | m-D14 | H | A-47 | A-47 | A-47 | H |
| 7474 | 0 | A-1 | H | m-D1 | m-D1 | m-D1 | H |
| 7475 | 0 | A-2 | H | m-D1 | m-D1 | m-D1 | H |
| 7476 | 0 | A-3 | H | m-D1 | m-D1 | m-D1 | H |
| 7477 | 0 | A-4 | H | m-D1 | m-D1 | m-D1 | H |
| 7478 | 0 | A-5 | H | m-D1 | m-D1 | m-D1 | H |
| 7479 | 0 | A-6 | H | m-D1 | m-D1 | m-D1 | H |
| 7480 | 0 | A-7 | H | m-D1 | m-D1 | m-D1 | H |
| 7481 | 0 | A-8 | H | m-D1 | m-D1 | m-D1 | H |
| 7482 | 0 | A-9 | H | m-D1 | m-D1 | m-D1 | H |
| 7483 | 0 | A-10 | H | m-D1 | m-D1 | m-D1 | H |
| 7484 | 0 | A-11 | H | m-D1 | m-D1 | m-D1 | H |
| 7485 | 0 | A-12 | H | m-D1 | m-D1 | m-D1 | H |
| 7486 | 0 | A-13 | H | m-D1 | m-D1 | m-D1 | H |
| 7487 | 0 | A-14 | H | m-D1 | m-D1 | m-D1 | H |
| 7488 | 0 | A-15 | H | m-D1 | m-D1 | m-D1 | H |
| 7489 | 0 | A-16 | H | m-D1 | m-D1 | m-D1 | H |
| 7490 | 0 | A-17 | H | m-D1 | m-D1 | m-D1 | H |
| 7491 | 0 | A-18 | H | m-D1 | m-D1 | m-D1 | H |
| 7492 | 0 | A-19 | H | m-D1 | m-D1 | m-D1 | H |
| 7493 | 0 | A-20 | H | m-D1 | m-D1 | m-D1 | H |
| 7494 | 0 | A-21 | H | m-D1 | m-D1 | m-D1 | H |
| 7495 | 0 | A-22 | H | m-D1 | m-D1 | m-D1 | H |
| 7496 | 0 | A-23 | H | m-D1 | m-D1 | m-D1 | H |
| 7497 | 0 | A-24 | H | m-D1 | m-D1 | m-D1 | H |
| 7498 | 0 | A-25 | H | m-D1 | m-D1 | m-D1 | H |
| 7499 | 0 | A-26 | H | m-D1 | m-D1 | m-D1 | H |
| 7500 | 0 | A-27 | H | m-D1 | m-D1 | m-D1 | H |
| 7501 | 0 | A-28 | H | m-D1 | m-D1 | m-D1 | H |
| 7502 | 0 | A-29 | H | m-D1 | m-D1 | m-D1 | H |
| 7503 | 0 | A-30 | H | m-D1 | m-D1 | m-D1 | H |
| 7504 | 0 | A-31 | H | m-D1 | m-D1 | m-D1 | H |
| 7505 | 0 | A-32 | H | m-D1 | m-D1 | m-D1 | H |
| 7506 | 0 | A-33 | H | m-D1 | m-D1 | m-D1 | H |
| 7507 | 0 | A-34 | H | m-D1 | m-D1 | m-D1 | H |
| 7508 | 0 | A-35 | H | m-D1 | m-D1 | m-D1 | H |
| 7509 | 0 | A-36 | H | m-D1 | m-D1 | m-D1 | H |
| 7510 | 0 | A-37 | H | m-D1 | m-D1 | m-D1 | H |
| 7511 | 0 | A-38 | H | m-D1 | m-D1 | m-D1 | H |
| 7512 | 0 | A-39 | H | m-D1 | m-D1 | m-D1 | H |
| 7513 | 0 | A-40 | H | m-D1 | m-D1 | m-D1 | H |
| 7514 | 0 | A-41 | H | m-D1 | m-D1 | m-D1 | H |
| 7515 | 0 | A-42 | H | m-D1 | m-D1 | m-D1 | H |
| 7516 | 0 | A-43 | H | m-D1 | m-D1 | m-D1 | H |
| 7517 | 0 | A-44 | H | m-D1 | m-D1 | m-D1 | H |
| 7518 | 0 | A-45 | H | m-D1 | m-D1 | m-D1 | H |
| 7519 | 0 | A-46 | H | m-D1 | m-D1 | m-D1 | H |
| 7520 | 0 | A-47 | H | m-D1 | m-D1 | m-D1 | H |
| 7521 | 0 | A-1 | H | m-D2 | m-D2 | m-D2 | H |
| 7522 | 0 | A-2 | H | m-D2 | m-D2 | m-D2 | H |
| 7523 | 0 | A-3 | H | m-D2 | m-D2 | m-D2 | H |
| 7524 | 0 | A-4 | H | m-D2 | m-D2 | m-D2 | H |
| 7525 | 0 | A-5 | H | m-D2 | m-D2 | m-D2 | H |
| 7526 | 0 | A-6 | H | m-D2 | m-D2 | m-D2 | H |
| 7527 | 0 | A-7 | H | m-D2 | m-D2 | m-D2 | H |
| 7528 | 0 | A-8 | H | m-D2 | m-D2 | m-D2 | H |
| 7529 | 0 | A-9 | H | m-D2 | m-D2 | m-D2 | H |
| 7530 | 0 | A-10 | H | m-D2 | m-D2 | m-D2 | H |
| 7531 | 0 | A-11 | H | m-D2 | m-D2 | m-D2 | H |
| 7532 | 0 | A-12 | H | m-D2 | m-D2 | m-D2 | H |
| 7533 | 0 | A-13 | H | m-D2 | m-D2 | m-D2 | H |
| 7534 | 0 | A-14 | H | m-D2 | m-D2 | m-D2 | H |
| 7535 | 0 | A-15 | H | m-D2 | m-D2 | m-D2 | H |
| 7536 | 0 | A-16 | H | m-D2 | m-D2 | m-D2 | H |
| 7537 | 0 | A-17 | H | m-D2 | m-D2 | m-D2 | H |
| 7538 | 0 | A-18 | H | m-D2 | m-D2 | m-D2 | H |
| 7539 | 0 | A-19 | H | m-D2 | m-D2 | m-D2 | H |
| 7540 | 0 | A-20 | H | m-D2 | m-D2 | m-D2 | H |
| 7541 | 0 | A-21 | H | m-D2 | m-D2 | m-D2 | H |
| 7542 | 0 | A-22 | H | m-D2 | m-D2 | m-D2 | H |
| 7543 | 0 | A-23 | H | m-D2 | m-D2 | m-D2 | H |
| 7544 | 0 | A-24 | H | m-D2 | m-D2 | m-D2 | H |
| 7545 | 0 | A-25 | H | m-D2 | m-D2 | m-D2 | H |
| 7546 | 0 | A-26 | H | m-D2 | m-D2 | m-D2 | H |
| 7547 | 0 | A-27 | H | m-D2 | m-D2 | m-D2 | H |
| 7548 | 0 | A-28 | H | m-D2 | m-D2 | m-D2 | H |
| 7549 | 0 | A-29 | H | m-D2 | m-D2 | m-D2 | H |
| 7550 | 0 | A-30 | H | m-D2 | m-D2 | m-D2 | H |
| 7551 | 0 | A-31 | H | m-D2 | m-D2 | m-D2 | H |
| 7552 | 0 | A-32 | H | m-D2 | m-D2 | m-D2 | H |
| 7553 | 0 | A-33 | H | m-D2 | m-D2 | m-D2 | H |
| 7554 | 0 | A-34 | H | m-D2 | m-D2 | m-D2 | H |
| 7555 | 0 | A-35 | H | m-D2 | m-D2 | m-D2 | H |
| 7556 | 0 | A-36 | H | m-D2 | m-D2 | m-D2 | H |
| 7557 | 0 | A-37 | H | m-D2 | m-D2 | m-D2 | H |
| 7558 | 0 | A-38 | H | m-D2 | m-D2 | m-D2 | H |
| 7559 | 0 | A-39 | H | m-D2 | m-D2 | m-D2 | H |
| 7560 | 0 | A-40 | H | m-D2 | m-D2 | m-D2 | H |
| 7561 | 0 | A-41 | H | m-D2 | m-D2 | m-D2 | H |
| 7562 | 0 | A-42 | H | m-D2 | m-D2 | m-D2 | H |
| 7563 | 0 | A-43 | H | m-D2 | m-D2 | m-D2 | H |
| 7564 | 0 | A-44 | H | m-D2 | m-D2 | m-D2 | H |
| 7565 | 0 | A-45 | H | m-D2 | m-D2 | m-D2 | H |
| 7566 | 0 | A-46 | H | m-D2 | m-D2 | m-D2 | H |
| 7567 | 0 | A-47 | H | m-D2 | m-D2 | m-D2 | H |
| 7568 | 0 | A-1 | H | m-D3 | m-D3 | m-D3 | H |
| 7569 | 0 | A-2 | H | m-D3 | m-D3 | m-D3 | H |
| 7570 | 0 | A-3 | H | m-D3 | m-D3 | m-D3 | H |
| 7571 | 0 | A-4 | H | m-D3 | m-D3 | m-D3 | H |
| 7572 | 0 | A-5 | H | m-D3 | m-D3 | m-D3 | H |
| 7573 | 0 | A-6 | H | m-D3 | m-D3 | m-D3 | H |
| 7574 | 0 | A-7 | H | m-D3 | m-D3 | m-D3 | H |
| 7575 | 0 | A-8 | H | m-D3 | m-D3 | m-D3 | H |
| 7576 | 0 | A-9 | H | m-D3 | m-D3 | m-D3 | H |
| 7577 | 0 | A-10 | H | m-D3 | m-D3 | m-D3 | H |
| 7578 | 0 | A-11 | H | m-D3 | m-D3 | m-D3 | H |
| 7579 | 0 | A-12 | H | m-D3 | m-D3 | m-D3 | H |
| 7580 | 0 | A-13 | H | m-D3 | m-D3 | m-D3 | H |
| 7581 | 0 | A-14 | H | m-D3 | m-D3 | m-D3 | H |
| 7582 | 0 | A-15 | H | m-D3 | m-D3 | m-D3 | H |
| 7583 | 0 | A-16 | H | m-D3 | m-D3 | m-D3 | H |
| 7584 | 0 | A-17 | H | m-D3 | m-D3 | m-D3 | H |
| 7585 | 0 | A-18 | H | m-D3 | m-D3 | m-D3 | H |
| 7586 | 0 | A-19 | H | m-D3 | m-D3 | m-D3 | H |
| 7587 | 0 | A-20 | H | m-D3 | m-D3 | m-D3 | H |
| 7588 | 0 | A-21 | H | m-D3 | m-D3 | m-D3 | H |
| 7589 | 0 | A-22 | H | m-D3 | m-D3 | m-D3 | H |
| 7590 | 0 | A-23 | H | m-D3 | m-D3 | m-D3 | H |
| 7591 | 0 | A-24 | H | m-D3 | m-D3 | m-D3 | H |
| 7592 | 0 | A-25 | H | m-D3 | m-D3 | m-D3 | H |
| 7593 | 0 | A-26 | H | m-D3 | m-D3 | m-D3 | H |
| 7594 | 0 | A-27 | H | m-D3 | m-D3 | m-D3 | H |
| 7595 | 0 | A-28 | H | m-D3 | m-D3 | m-D3 | H |
| 7596 | 0 | A-29 | H | m-D3 | m-D3 | m-D3 | H |
| 7597 | 0 | A-30 | H | m-D3 | m-D3 | m-D3 | H |
| 7598 | 0 | A-31 | H | m-D3 | m-D3 | m-D3 | H |
| 7599 | 0 | A-32 | H | m-D3 | m-D3 | m-D3 | H |
| 7600 | 0 | A-33 | H | m-D3 | m-D3 | m-D3 | H |
| 7601 | 0 | A-34 | H | m-D3 | m-D3 | m-D3 | H |
| 7602 | 0 | A-35 | H | m-D3 | m-D3 | m-D3 | H |
| 7603 | 0 | A-36 | H | m-D3 | m-D3 | m-D3 | H |
| 7604 | 0 | A-37 | H | m-D3 | m-D3 | m-D3 | H |
| 7605 | 0 | A-38 | H | m-D3 | m-D3 | m-D3 | H |
| 7606 | 0 | A-39 | H | m-D3 | m-D3 | m-D3 | H |
| 7607 | 0 | A-40 | H | m-D3 | m-D3 | m-D3 | H |
| 7608 | 0 | A-41 | H | m-D3 | m-D3 | m-D3 | H |
| 7609 | 0 | A-42 | H | m-D3 | m-D3 | m-D3 | H |
| 7610 | 0 | A-43 | H | m-D3 | m-D3 | m-D3 | H |
| 7611 | 0 | A-44 | H | m-D3 | m-D3 | m-D3 | H |
| 7612 | 0 | A-45 | H | m-D3 | m-D3 | m-D3 | H |
| 7613 | 0 | A-46 | H | m-D3 | m-D3 | m-D3 | H |
| 7614 | 0 | A-47 | H | m-D3 | m-D3 | m-D3 | H |
| 7615 | 0 | A-1 | H | m-D5 | m-D5 | m-D5 | H |
| 7616 | 0 | A-2 | H | m-D5 | m-D5 | m-D5 | H |
| 7617 | 0 | A-3 | H | m-D5 | m-D5 | m-D5 | H |
| 7618 | 0 | A-4 | H | m-D5 | m-D5 | m-D5 | H |
| 7619 | 0 | A-5 | H | m-D5 | m-D5 | m-D5 | H |
| 7620 | 0 | A-6 | H | m-D5 | m-D5 | m-D5 | H |
| 7621 | 0 | A-7 | H | m-D5 | m-D5 | m-D5 | H |
| 7622 | 0 | A-8 | H | m-D5 | m-D5 | m-D5 | H |
| 7623 | 0 | A-9 | H | m-D5 | m-D5 | m-D5 | H |
| 7624 | 0 | A-10 | H | m-D5 | m-D5 | m-D5 | H |
| 7625 | 0 | A-11 | H | m-D5 | m-D5 | m-D5 | H |
| 7626 | 0 | A-12 | H | m-D5 | m-D5 | m-D5 | H |
| 7627 | 0 | A-13 | H | m-D5 | m-D5 | m-D5 | H |
| 7628 | 0 | A-14 | H | m-D5 | m-D5 | m-D5 | H |
| 7629 | 0 | A-15 | H | m-D5 | m-D5 | m-D5 | H |
| 7630 | 0 | A-16 | H | m-D5 | m-D5 | m-D5 | H |
| 7631 | 0 | A-17 | H | m-D5 | m-D5 | m-D5 | H |
| 7632 | 0 | A-18 | H | m-D5 | m-D5 | m-D5 | H |
| 7633 | 0 | A-19 | H | m-D5 | m-D5 | m-D5 | H |
| 7634 | 0 | A-20 | H | m-D5 | m-D5 | m-D5 | H |
| 7635 | 0 | A-21 | H | m-D5 | m-D5 | m-D5 | H |
| 7636 | 0 | A-22 | H | m-D5 | m-D5 | m-D5 | H |
| 7637 | 0 | A-23 | H | m-D5 | m-D5 | m-D5 | H |
| 7638 | 0 | A-24 | H | m-D5 | m-D5 | m-D5 | H |
| 7639 | 0 | A-25 | H | m-D5 | m-D5 | m-D5 | H |
| 7640 | 0 | A-26 | H | m-D5 | m-D5 | m-D5 | H |
| 7641 | 0 | A-27 | H | m-D5 | m-D5 | m-D5 | H |
| 7642 | 0 | A-28 | H | m-D5 | m-D5 | m-D5 | H |
| 7643 | 0 | A-29 | H | m-D5 | m-D5 | m-D5 | H |
| 7644 | 0 | A-30 | H | m-D5 | m-D5 | m-D5 | H |
| 7645 | 0 | A-31 | H | m-D5 | m-D5 | m-D5 | H |
| 7646 | 0 | A-32 | H | m-D5 | m-D5 | m-D5 | H |
| 7647 | 0 | A-33 | H | m-D5 | m-D5 | m-D5 | H |
| 7648 | 0 | A-34 | H | m-D5 | m-D5 | m-D5 | H |
| 7649 | 0 | A-35 | H | m-D5 | m-D5 | m-D5 | H |
| 7650 | 0 | A-36 | H | m-D5 | m-D5 | m-D5 | H |
| 7651 | 0 | A-37 | H | m-D5 | m-D5 | m-D5 | H |
| 7652 | 0 | A-38 | H | m-D5 | m-D5 | m-D5 | H |
| 7653 | 0 | A-39 | H | m-D5 | m-D5 | m-D5 | H |
| 7654 | 0 | A-40 | H | m-D5 | m-D5 | m-D5 | H |
| 7655 | 0 | A-41 | H | m-D5 | m-D5 | m-D5 | H |
| 7656 | 0 | A-42 | H | m-D5 | m-D5 | m-D5 | H |
| 7657 | 0 | A-43 | H | m-D5 | m-D5 | m-D5 | H |
| 7658 | 0 | A-44 | H | m-D5 | m-D5 | m-D5 | H |
| 7659 | 0 | A-45 | H | m-D5 | m-D5 | m-D5 | H |
| 7660 | 0 | A-46 | H | m-D5 | m-D5 | m-D5 | H |
| 7661 | 0 | A-47 | H | m-D5 | m-D5 | m-D5 | H |
| 7662 | 0 | A-1 | H | m-D9 | m-D9 | m-D9 | H |
| 7663 | 0 | A-2 | H | m-D9 | m-D9 | m-D9 | H |
| 7664 | 0 | A-3 | H | m-D9 | m-D9 | m-D9 | H |
| 7665 | 0 | A-4 | H | m-D9 | m-D9 | m-D9 | H |
| 7666 | 0 | A-5 | H | m-D9 | m-D9 | m-D9 | H |
| 7667 | 0 | A-6 | H | m-D9 | m-D9 | m-D9 | H |
| 7668 | 0 | A-7 | H | m-D9 | m-D9 | m-D9 | H |
| 7669 | 0 | A-8 | H | m-D9 | m-D9 | m-D9 | H |
| 7670 | 0 | A-9 | H | m-D9 | m-D9 | m-D9 | H |
| 7671 | 0 | A-10 | H | m-D9 | m-D9 | m-D9 | H |
| 7672 | 0 | A-11 | H | m-D9 | m-D9 | m-D9 | H |
| 7673 | 0 | A-12 | H | m-D9 | m-D9 | m-D9 | H |
| 7674 | 0 | A-13 | H | m-D9 | m-D9 | m-D9 | H |
| 7675 | 0 | A-14 | H | m-D9 | m-D9 | m-D9 | H |
| 7676 | 0 | A-15 | H | m-D9 | m-D9 | m-D9 | H |
| 7677 | 0 | A-16 | H | m-D9 | m-D9 | m-D9 | H |
| 7678 | 0 | A-17 | H | m-D9 | m-D9 | m-D9 | H |
| 7679 | 0 | A-18 | H | m-D9 | m-D9 | m-D9 | H |
| 7680 | 0 | A-19 | H | m-D9 | m-D9 | m-D9 | H |
| 7681 | 0 | A-20 | H | m-D9 | m-D9 | m-D9 | H |
| 7682 | 0 | A-21 | H | m-D9 | m-D9 | m-D9 | H |
| 7683 | 0 | A-22 | H | m-D9 | m-D9 | m-D9 | H |
| 7684 | 0 | A-23 | H | m-D9 | m-D9 | m-D9 | H |
| 7685 | 0 | A-24 | H | m-D9 | m-D9 | m-D9 | H |
| 7686 | 0 | A-25 | H | m-D9 | m-D9 | m-D9 | H |
| 7687 | 0 | A-26 | H | m-D9 | m-D9 | m-D9 | H |
| 7688 | 0 | A-27 | H | m-D9 | m-D9 | m-D9 | H |
| 7689 | 0 | A-28 | H | m-D9 | m-D9 | m-D9 | H |
| 7690 | 0 | A-29 | H | m-D9 | m-D9 | m-D9 | H |
| 7691 | 0 | A-30 | H | m-D9 | m-D9 | m-D9 | H |
| 7692 | 0 | A-31 | H | m-D9 | m-D9 | m-D9 | H |
| 7693 | 0 | A-32 | H | m-D9 | m-D9 | m-D9 | H |
| 7694 | 0 | A-33 | H | m-D9 | m-D9 | m-D9 | H |
| 7695 | 0 | A-34 | H | m-D9 | m-D9 | m-D9 | H |
| 7696 | 0 | A-35 | H | m-D9 | m-D9 | m-D9 | H |
| 7697 | 0 | A-36 | H | m-D9 | m-D9 | m-D9 | H |
| 7698 | 0 | A-37 | H | m-D9 | m-D9 | m-D9 | H |
| 7699 | 0 | A-38 | H | m-D9 | m-D9 | m-D9 | H |
| 7700 | 0 | A-39 | H | m-D9 | m-D9 | m-D9 | H |
| 7701 | 0 | A-40 | H | m-D9 | m-D9 | m-D9 | H |
| 7702 | 0 | A-41 | H | m-D9 | m-D9 | m-D9 | H |
| 7703 | 0 | A-42 | H | m-D9 | m-D9 | m-D9 | H |
| 7704 | 0 | A-43 | H | m-D9 | m-D9 | m-D9 | H |
| 7705 | 0 | A-44 | H | m-D9 | m-D9 | m-D9 | H |
| 7706 | 0 | A-45 | H | m-D9 | m-D9 | m-D9 | H |
| 7707 | 0 | A-46 | H | m-D9 | m-D9 | m-D9 | H |
| 7708 | 0 | A-47 | H | m-D9 | m-D9 | m-D9 | H |
| 7709 | 0 | A-1 | H | m-D11 | m-D11 | m-D11 | H |
| 7710 | 0 | A-2 | H | m-D11 | m-D11 | m-D11 | H |
| 7711 | 0 | A-3 | H | m-D11 | m-D11 | m-D11 | H |
| 7712 | 0 | A-4 | H | m-D11 | m-D11 | m-D11 | H |
| 7713 | 0 | A-5 | H | m-D11 | m-D11 | m-D11 | H |
| 7714 | 0 | A-6 | H | m-D11 | m-D11 | m-D11 | H |
| 7715 | 0 | A-7 | H | m-D11 | m-D11 | m-D11 | H |
| 7716 | 0 | A-8 | H | m-D11 | m-D11 | m-D11 | H |
| 7717 | 0 | A-9 | H | m-D11 | m-D11 | m-D11 | H |
| 7718 | 0 | A-10 | H | m-D11 | m-D11 | m-D11 | H |
| 7719 | 0 | A-11 | H | m-D11 | m-D11 | m-D11 | H |
| 7720 | 0 | A-12 | H | m-D11 | m-D11 | m-D11 | H |
| 7721 | 0 | A-13 | H | m-D11 | m-D11 | m-D11 | H |
| 7722 | 0 | A-14 | H | m-D11 | m-D11 | m-D11 | H |
| 7723 | 0 | A-15 | H | m-D11 | m-D11 | m-D11 | H |
| 7724 | 0 | A-16 | H | m-D11 | m-D11 | m-D11 | H |
| 7725 | 0 | A-17 | H | m-D11 | m-D11 | m-D11 | H |
| 7726 | 0 | A-18 | H | m-D11 | m-D11 | m-D11 | H |
| 7727 | 0 | A-19 | H | m-D11 | m-D11 | m-D11 | H |
| 7728 | 0 | A-20 | H | m-D11 | m-D11 | m-D11 | H |
| 7729 | 0 | A-21 | H | m-D11 | m-D11 | m-D11 | H |
| 7730 | 0 | A-22 | H | m-D11 | m-D11 | m-D11 | H |
| 7731 | 0 | A-23 | H | m-D11 | m-D11 | m-D11 | H |
| 7732 | 0 | A-24 | H | m-D11 | m-D11 | m-D11 | H |
| 7733 | 0 | A-25 | H | m-D11 | m-D11 | m-D11 | H |
| 7734 | 0 | A-26 | H | m-D11 | m-D11 | m-D11 | H |
| 7735 | 0 | A-27 | H | m-D11 | m-D11 | m-D11 | H |
| 7736 | 0 | A-28 | H | m-D11 | m-D11 | m-D11 | H |
| 7737 | 0 | A-29 | H | m-D11 | m-D11 | m-D11 | H |
| 7738 | 0 | A-30 | H | m-D11 | m-D11 | m-D11 | H |
| 7739 | 0 | A-31 | H | m-D11 | m-D11 | m-D11 | H |
| 7740 | 0 | A-32 | H | m-D11 | m-D11 | m-D11 | H |
| 7741 | 0 | A-33 | H | m-D11 | m-D11 | m-D11 | H |
| 7742 | 0 | A-34 | H | m-D11 | m-D11 | m-D11 | H |
| 7743 | 0 | A-35 | H | m-D11 | m-D11 | m-D11 | H |
| 7744 | 0 | A-36 | H | m-D11 | m-D11 | m-D11 | H |
| 7745 | 0 | A-37 | H | m-D11 | m-D11 | m-D11 | H |
| 7746 | 0 | A-38 | H | m-D11 | m-D11 | m-D11 | H |
| 7747 | 0 | A-39 | H | m-D11 | m-D11 | m-D11 | H |
| 7748 | 0 | A-40 | H | m-D11 | m-D11 | m-D11 | H |
| 7749 | 0 | A-41 | H | m-D11 | m-D11 | m-D11 | H |
| 7750 | 0 | A-42 | H | m-D11 | m-D11 | m-D11 | H |
| 7751 | 0 | A-43 | H | m-D11 | m-D11 | m-D11 | H |
| 7752 | 0 | A-44 | H | m-D11 | m-D11 | m-D11 | H |
| 7753 | 0 | A-45 | H | m-D11 | m-D11 | m-D11 | H |
| 7754 | 0 | A-46 | H | m-D11 | m-D11 | m-D11 | H |
| 7755 | 0 | A-47 | H | m-D11 | m-D11 | m-D11 | H |
| 7756 | 0 | A-1 | H | m-D12 | m-D12 | m-D12 | H |
| 7757 | 0 | A-2 | H | m-D12 | m-D12 | m-D12 | H |
| 7758 | 0 | A-3 | H | m-D12 | m-D12 | m-D12 | H |
| 7759 | 0 | A-4 | H | m-D12 | m-D12 | m-D12 | H |
| 7760 | 0 | A-5 | H | m-D12 | m-D12 | m-D12 | H |
| 7761 | 0 | A-6 | H | m-D12 | m-D12 | m-D12 | H |
| 7762 | 0 | A-7 | H | m-D12 | m-D12 | m-D12 | H |
| 7763 | 0 | A-8 | H | m-D12 | m-D12 | m-D12 | H |
| 7764 | 0 | A-9 | H | m-D12 | m-D12 | m-D12 | H |
| 7765 | 0 | A-10 | H | m-D12 | m-D12 | m-D12 | H |
| 7766 | 0 | A-11 | H | m-D12 | m-D12 | m-D12 | H |
| 7767 | 0 | A-12 | H | m-D12 | m-D12 | m-D12 | H |
| 7768 | 0 | A-13 | H | m-D12 | m-D12 | m-D12 | H |
| 7769 | 0 | A-14 | H | m-D12 | m-D12 | m-D12 | H |
| 7770 | 0 | A-15 | H | m-D12 | m-D12 | m-D12 | H |
| 7771 | 0 | A-16 | H | m-D12 | m-D12 | m-D12 | H |
| 7772 | 0 | A-17 | H | m-D12 | m-D12 | m-D12 | H |
| 7773 | 0 | A-18 | H | m-D12 | m-D12 | m-D12 | H |
| 7774 | 0 | A-19 | H | m-D12 | m-D12 | m-D12 | H |
| 7775 | 0 | A-20 | H | m-D12 | m-D12 | m-D12 | H |
| 7776 | 0 | A-21 | H | m-D12 | m-D12 | m-D12 | H |
| 7777 | 0 | A-22 | H | m-D12 | m-D12 | m-D12 | H |
| 7778 | 0 | A-23 | H | m-D12 | m-D12 | m-D12 | H |
| 7779 | 0 | A-24 | H | m-D12 | m-D12 | m-D12 | H |
| 7780 | 0 | A-25 | H | m-D12 | m-D12 | m-D12 | H |
| 7781 | 0 | A-26 | H | m-D12 | m-D12 | m-D12 | H |
| 7782 | 0 | A-27 | H | m-D12 | m-D12 | m-D12 | H |
| 7783 | 0 | A-28 | H | m-D12 | m-D12 | m-D12 | H |
| 7784 | 0 | A-29 | H | m-D12 | m-D12 | m-D12 | H |
| 7785 | 0 | A-30 | H | m-D12 | m-D12 | m-D12 | H |
| 7786 | 0 | A-31 | H | m-D12 | m-D12 | m-D12 | H |
| 7787 | 0 | A-32 | H | m-D12 | m-D12 | m-D12 | H |
| 7788 | 0 | A-33 | H | m-D12 | m-D12 | m-D12 | H |
| 7789 | 0 | A-34 | H | m-D12 | m-D12 | m-D12 | H |
| 7790 | 0 | A-35 | H | m-D12 | m-D12 | m-D12 | H |
| 7791 | 0 | A-36 | H | m-D12 | m-D12 | m-D12 | H |
| 7792 | 0 | A-37 | H | m-D12 | m-D12 | m-D12 | H |
| 7793 | 0 | A-38 | H | m-D12 | m-D12 | m-D12 | H |
| 7794 | 0 | A-39 | H | m-D12 | m-D12 | m-D12 | H |
| 7795 | 0 | A-40 | H | m-D12 | m-D12 | m-D12 | H |
| 7796 | 0 | A-41 | H | m-D12 | m-D12 | m-D12 | H |
| 7797 | 0 | A-42 | H | m-D12 | m-D12 | m-D12 | H |
| 7798 | 0 | A-43 | H | m-D12 | m-D12 | m-D12 | H |
| 7799 | 0 | A-44 | H | m-D12 | m-D12 | m-D12 | H |
| 7800 | 0 | A-45 | H | m-D12 | m-D12 | m-D12 | H |
| 7801 | 0 | A-46 | H | m-D12 | m-D12 | m-D12 | H |
| 7802 | 0 | A-47 | H | m-D12 | m-D12 | m-D12 | H |
| 7803 | 0 | A-1 | H | m-D13 | m-D13 | m-D13 | H |
| 7804 | 0 | A-2 | H | m-D13 | m-D13 | m-D13 | H |
| 7805 | 0 | A-3 | H | m-D13 | m-D13 | m-D13 | H |
| 7806 | 0 | A-4 | H | m-D13 | m-D13 | m-D13 | H |
| 7807 | 0 | A-5 | H | m-D13 | m-D13 | m-D13 | H |
| 7808 | 0 | A-6 | H | m-D13 | m-D13 | m-D13 | H |
| 7809 | 0 | A-7 | H | m-D13 | m-D13 | m-D13 | H |
| 7810 | 0 | A-8 | H | m-D13 | m-D13 | m-D13 | H |
| 7811 | 0 | A-9 | H | m-D13 | m-D13 | m-D13 | H |
| 7812 | 0 | A-10 | H | m-D13 | m-D13 | m-D13 | H |
| 7813 | 0 | A-11 | H | m-D13 | m-D13 | m-D13 | H |
| 7814 | 0 | A-12 | H | m-D13 | m-D13 | m-D13 | H |
| 7815 | 0 | A-13 | H | m-D13 | m-D13 | m-D13 | H |
| 7816 | 0 | A-14 | H | m-D13 | m-D13 | m-D13 | H |
| 7817 | 0 | A-15 | H | m-D13 | m-D13 | m-D13 | H |
| 7818 | 0 | A-16 | H | m-D13 | m-D13 | m-D13 | H |
| 7819 | 0 | A-17 | H | m-D13 | m-D13 | m-D13 | H |
| 7820 | 0 | A-18 | H | m-D13 | m-D13 | m-D13 | H |
| 7821 | 0 | A-19 | H | m-D13 | m-D13 | m-D13 | H |
| 7822 | 0 | A-20 | H | m-D13 | m-D13 | m-D13 | H |
| 7823 | 0 | A-21 | H | m-D13 | m-D13 | m-D13 | H |
| 7824 | 0 | A-22 | H | m-D13 | m-D13 | m-D13 | H |
| 7825 | 0 | A-23 | H | m-D13 | m-D13 | m-D13 | H |
| 7826 | 0 | A-24 | H | m-D13 | m-D13 | m-D13 | H |
| 7827 | 0 | A-25 | H | m-D13 | m-D13 | m-D13 | H |
| 7828 | 0 | A-26 | H | m-D13 | m-D13 | m-D13 | H |
| 7829 | 0 | A-27 | H | m-D13 | m-D13 | m-D13 | H |
| 7830 | 0 | A-28 | H | m-D13 | m-D13 | m-D13 | H |
| 7831 | 0 | A-29 | H | m-D13 | m-D13 | m-D13 | H |
| 7832 | 0 | A-30 | H | m-D13 | m-D13 | m-D13 | H |
| 7833 | 0 | A-31 | H | m-D13 | m-D13 | m-D13 | H |
| 7834 | 0 | A-32 | H | m-D13 | m-D13 | m-D13 | H |
| 7835 | 0 | A-33 | H | m-D13 | m-D13 | m-D13 | H |
| 7836 | 0 | A-34 | H | m-D13 | m-D13 | m-D13 | H |
| 7837 | 0 | A-35 | H | m-D13 | m-D13 | m-D13 | H |
| 7838 | 0 | A-36 | H | m-D13 | m-D13 | m-D13 | H |
| 7839 | 0 | A-37 | H | m-D13 | m-D13 | m-D13 | H |
| 7840 | 0 | A-38 | H | m-D13 | m-D13 | m-D13 | H |
| 7841 | 0 | A-39 | H | m-D13 | m-D13 | m-D13 | H |
| 7842 | 0 | A-40 | H | m-D13 | m-D13 | m-D13 | H |
| 7843 | 0 | A-41 | H | m-D13 | m-D13 | m-D13 | H |
| 7844 | 0 | A-42 | H | m-D13 | m-D13 | m-D13 | H |
| 7845 | 0 | A-43 | H | m-D13 | m-D13 | m-D13 | H |
| 7846 | 0 | A-44 | H | m-D13 | m-D13 | m-D13 | H |
| 7847 | 0 | A-45 | H | m-D13 | m-D13 | m-D13 | H |
| 7848 | 0 | A-46 | H | m-D13 | m-D13 | m-D13 | H |
| 7849 | 0 | A-47 | H | m-D13 | m-D13 | m-D13 | H |
| 7850 | 0 | A-1 | H | m-D14 | m-D14 | m-D14 | H |
| 7851 | 0 | A-2 | H | m-D14 | m-D14 | m-D14 | H |
| 7852 | 0 | A-3 | H | m-D14 | m-D14 | m-D14 | H |
| 7853 | 0 | A-4 | H | m-D14 | m-D14 | m-D14 | H |
| 7854 | 0 | A-5 | H | m-D14 | m-D14 | m-D14 | H |
| 7855 | 0 | A-6 | H | m-D14 | m-D14 | m-D14 | H |
| 7856 | 0 | A-7 | H | m-D14 | m-D14 | m-D14 | H |
| 7857 | 0 | A-8 | H | m-D14 | m-D14 | m-D14 | H |
| 7858 | 0 | A-9 | H | m-D14 | m-D14 | m-D14 | H |
| 7859 | 0 | A-10 | H | m-D14 | m-D14 | m-D14 | H |
| 7860 | 0 | A-11 | H | m-D14 | m-D14 | m-D14 | H |
| 7861 | 0 | A-12 | H | m-D14 | m-D14 | m-D14 | H |
| 7862 | 0 | A-13 | H | m-D14 | m-D14 | m-D14 | H |
| 7863 | 0 | A-14 | H | m-D14 | m-D14 | m-D14 | H |
| 7864 | 0 | A-15 | H | m-D14 | m-D14 | m-D14 | H |
| 7865 | 0 | A-16 | H | m-D14 | m-D14 | m-D14 | H |
| 7866 | 0 | A-17 | H | m-D14 | m-D14 | m-D14 | H |
| 7867 | 0 | A-18 | H | m-D14 | m-D14 | m-D14 | H |
| 7868 | 0 | A-19 | H | m-D14 | m-D14 | m-D14 | H |
| 7869 | 0 | A-20 | H | m-D14 | m-D14 | m-D14 | H |
| 7870 | 0 | A-21 | H | m-D14 | m-D14 | m-D14 | H |
| 7871 | 0 | A-22 | H | m-D14 | m-D14 | m-D14 | H |
| 7872 | 0 | A-23 | H | m-D14 | m-D14 | m-D14 | H |
| 7873 | 0 | A-24 | H | m-D14 | m-D14 | m-D14 | H |
| 7874 | 0 | A-25 | H | m-D14 | m-D14 | m-D14 | H |
| 7875 | 0 | A-26 | H | m-D14 | m-D14 | m-D14 | H |
| 7876 | 0 | A-27 | H | m-D14 | m-D14 | m-D14 | H |
| 7877 | 0 | A-28 | H | m-D14 | m-D14 | m-D14 | H |
| 7878 | 0 | A-29 | H | m-D14 | m-D14 | m-D14 | H |
| 7879 | 0 | A-30 | H | m-D14 | m-D14 | m-D14 | H |
| 7880 | 0 | A-31 | H | m-D14 | m-D14 | m-D14 | H |
| 7881 | 0 | A-32 | H | m-D14 | m-D14 | m-D14 | H |
| 7882 | 0 | A-33 | H | m-D14 | m-D14 | m-D14 | H |
| 7883 | 0 | A-34 | H | m-D14 | m-D14 | m-D14 | H |
| 7884 | 0 | A-35 | H | m-D14 | m-D14 | m-D14 | H |
| 7885 | 0 | A-36 | H | m-D14 | m-D14 | m-D14 | H |
| 7886 | 0 | A-37 | H | m-D14 | m-D14 | m-D14 | H |
| 7887 | 0 | A-38 | H | m-D14 | m-D14 | m-D14 | H |
| 7888 | 0 | A-39 | H | m-D14 | m-D14 | m-D14 | H |
| 7889 | 0 | A-40 | H | m-D14 | m-D14 | m-D14 | H |
| 7890 | 0 | A-41 | H | m-D14 | m-D14 | m-D14 | H |
| 7891 | 0 | A-42 | H | m-D14 | m-D14 | m-D14 | H |
| 7892 | 0 | A-43 | H | m-D14 | m-D14 | m-D14 | H |
| 7893 | 0 | A-44 | H | m-D14 | m-D14 | m-D14 | H |
| 7894 | 0 | A-45 | H | m-D14 | m-D14 | m-D14 | H |
| 7895 | 0 | A-46 | H | m-D14 | m-D14 | m-D14 | H |
| 7896 | 0 | A-47 | H | m-D14 | m-D14 | m-D14 | H |
| 7897 | 0 | A-1 | H | m-D1 | A-1 | m-D1 | H |
| 7898 | 0 | A-2 | H | m-D1 | A-2 | m-D1 | H |
| 7899 | 0 | A-3 | H | m-D1 | A-3 | m-D1 | H |
| 7900 | 0 | A-4 | H | m-D1 | A-4 | m-D1 | H |
| 7901 | 0 | A-5 | H | m-D1 | A-5 | m-D1 | H |
| 7902 | 0 | A-6 | H | m-D1 | A-6 | m-D1 | H |
| 7903 | 0 | A-7 | H | m-D1 | A-7 | m-D1 | H |
| 7904 | 0 | A-8 | H | m-D1 | A-8 | m-D1 | H |
| 7905 | 0 | A-9 | H | m-D1 | A-9 | m-D1 | H |
| 7906 | 0 | A-10 | H | m-D1 | A-10 | m-D1 | H |
| 7907 | 0 | A-11 | H | m-D1 | A-11 | m-D1 | H |
| 7908 | 0 | A-12 | H | m-D1 | A-12 | m-D1 | H |
| 7909 | 0 | A-13 | H | m-D1 | A-13 | m-D1 | H |
| 7910 | 0 | A-14 | H | m-D1 | A-14 | m-D1 | H |
| 7911 | 0 | A-15 | H | m-D1 | A-15 | m-D1 | H |
| 7912 | 0 | A-16 | H | m-D1 | A-16 | m-D1 | H |
| 7913 | 0 | A-17 | H | m-D1 | A-17 | m-D1 | H |
| 7914 | 0 | A-18 | H | m-D1 | A-18 | m-D1 | H |
| 7915 | 0 | A-19 | H | m-D1 | A-19 | m-D1 | H |
| 7916 | 0 | A-20 | H | m-D1 | A-20 | m-D1 | H |
| 7917 | 0 | A-21 | H | m-D1 | A-21 | m-D1 | H |
| 7918 | 0 | A-22 | H | m-D1 | A-22 | m-D1 | H |
| 7919 | 0 | A-23 | H | m-D1 | A-23 | m-D1 | H |
| 7920 | 0 | A-24 | H | m-D1 | A-24 | m-D1 | H |
| 7921 | 0 | A-25 | H | m-D1 | A-25 | m-D1 | H |
| 7922 | 0 | A-26 | H | m-D1 | A-26 | m-D1 | H |
| 7923 | 0 | A-27 | H | m-D1 | A-27 | m-D1 | H |
| 7924 | 0 | A-28 | H | m-D1 | A-28 | m-D1 | H |
| 7925 | 0 | A-29 | H | m-D1 | A-29 | m-D1 | H |
| 7926 | 0 | A-30 | H | m-D1 | A-30 | m-D1 | H |
| 7927 | 0 | A-31 | H | m-D1 | A-31 | m-D1 | H |
| 7928 | 0 | A-32 | H | m-D1 | A-32 | m-D1 | H |
| 7929 | 0 | A-33 | H | m-D1 | A-33 | m-D1 | H |
| 7930 | 0 | A-34 | H | m-D1 | A-34 | m-D1 | H |
| 7931 | 0 | A-35 | H | m-D1 | A-35 | m-D1 | H |
| 7932 | 0 | A-36 | H | m-D1 | A-36 | m-D1 | H |
| 7933 | 0 | A-37 | H | m-D1 | A-37 | m-D1 | H |
| 7934 | 0 | A-38 | H | m-D1 | A-38 | m-D1 | H |
| 7935 | 0 | A-39 | H | m-D1 | A-39 | m-D1 | H |
| 7936 | 0 | A-40 | H | m-D1 | A-40 | m-D1 | H |
| 7937 | 0 | A-41 | H | m-D1 | A-41 | m-D1 | H |
| 7938 | 0 | A-42 | H | m-D1 | A-42 | m-D1 | H |
| 7939 | 0 | A-43 | H | m-D1 | A-43 | m-D1 | H |
| 7940 | 0 | A-44 | H | m-D1 | A-44 | m-D1 | H |
| 7941 | 0 | A-45 | H | m-D1 | A-45 | m-D1 | H |
| 7942 | 0 | A-46 | H | m-D1 | A-46 | m-D1 | H |
| 7943 | 0 | A-47 | H | m-D1 | A-47 | m-D1 | H |
| 7944 | 0 | A-1 | H | m-D2 | A-1 | m-D2 | H |
| 7945 | 0 | A-2 | H | m-D2 | A-2 | m-D2 | H |
| 7946 | 0 | A-3 | H | m-D2 | A-3 | m-D2 | H |
| 7947 | 0 | A-4 | H | m-D2 | A-4 | m-D2 | H |
| 7948 | 0 | A-5 | H | m-D2 | A-5 | m-D2 | H |
| 7949 | 0 | A-6 | H | m-D2 | A-6 | m-D2 | H |
| 7950 | 0 | A-7 | H | m-D2 | A-7 | m-D2 | H |
| 7951 | 0 | A-8 | H | m-D2 | A-8 | m-D2 | H |
| 7952 | 0 | A-9 | H | m-D2 | A-9 | m-D2 | H |
| 7953 | 0 | A-10 | H | m-D2 | A-10 | m-D2 | H |
| 7954 | 0 | A-11 | H | m-D2 | A-11 | m-D2 | H |
| 7955 | 0 | A-12 | H | m-D2 | A-12 | m-D2 | H |
| 7956 | 0 | A-13 | H | m-D2 | A-13 | m-D2 | H |
| 7957 | 0 | A-14 | H | m-D2 | A-14 | m-D2 | H |
| 7958 | 0 | A-15 | H | m-D2 | A-15 | m-D2 | H |
| 7959 | 0 | A-16 | H | m-D2 | A-16 | m-D2 | H |
| 7960 | 0 | A-17 | H | m-D2 | A-17 | m-D2 | H |
| 7961 | 0 | A-18 | H | m-D2 | A-18 | m-D2 | H |
| 7962 | 0 | A-19 | H | m-D2 | A-19 | m-D2 | H |
| 7963 | 0 | A-20 | H | m-D2 | A-20 | m-D2 | H |
| 7964 | 0 | A-21 | H | m-D2 | A-21 | m-D2 | H |
| 7965 | 0 | A-22 | H | m-D2 | A-22 | m-D2 | H |
| 7966 | 0 | A-23 | H | m-D2 | A-23 | m-D2 | H |
| 7967 | 0 | A-24 | H | m-D2 | A-24 | m-D2 | H |
| 7968 | 0 | A-25 | H | m-D2 | A-25 | m-D2 | H |
| 7969 | 0 | A-26 | H | m-D2 | A-26 | m-D2 | H |
| 7970 | 0 | A-27 | H | m-D2 | A-27 | m-D2 | H |
| 7971 | 0 | A-28 | H | m-D2 | A-28 | m-D2 | H |
| 7972 | 0 | A-29 | H | m-D2 | A-29 | m-D2 | H |
| 7973 | 0 | A-30 | H | m-D2 | A-30 | m-D2 | H |
| 7974 | 0 | A-31 | H | m-D2 | A-31 | m-D2 | H |
| 7975 | 0 | A-32 | H | m-D2 | A-32 | m-D2 | H |
| 7976 | 0 | A-33 | H | m-D2 | A-33 | m-D2 | H |
| 7977 | 0 | A-34 | H | m-D2 | A-34 | m-D2 | H |
| 7978 | 0 | A-35 | H | m-D2 | A-35 | m-D2 | H |
| 7979 | 0 | A-36 | H | m-D2 | A-36 | m-D2 | H |
| 7980 | 0 | A-37 | H | m-D2 | A-37 | m-D2 | H |
| 7981 | 0 | A-38 | H | m-D2 | A-38 | m-D2 | H |
| 7982 | 0 | A-39 | H | m-D2 | A-39 | m-D2 | H |
| 7983 | 0 | A-40 | H | m-D2 | A-40 | m-D2 | H |
| 7984 | 0 | A-41 | H | m-D2 | A-41 | m-D2 | H |
| 7985 | 0 | A-42 | H | m-D2 | A-42 | m-D2 | H |
| 7986 | 0 | A-43 | H | m-D2 | A-43 | m-D2 | H |
| 7987 | 0 | A-44 | H | m-D2 | A-44 | m-D2 | H |
| 7988 | 0 | A-45 | H | m-D2 | A-45 | m-D2 | H |
| 7989 | 0 | A-46 | H | m-D2 | A-46 | m-D2 | H |
| 7990 | 0 | A-47 | H | m-D2 | A-47 | m-D2 | H |
| 7991 | 0 | A-1 | H | m-D3 | A-1 | m-D3 | H |
| 7992 | 0 | A-2 | H | m-D3 | A-2 | m-D3 | H |
| 7993 | 0 | A-3 | H | m-D3 | A-3 | m-D3 | H |
| 7994 | 0 | A-4 | H | m-D3 | A-4 | m-D3 | H |
| 7995 | 0 | A-5 | H | m-D3 | A-5 | m-D3 | H |
| 7996 | 0 | A-6 | H | m-D3 | A-6 | m-D3 | H |
| 7997 | 0 | A-7 | H | m-D3 | A-7 | m-D3 | H |
| 7998 | 0 | A-8 | H | m-D3 | A-8 | m-D3 | H |
| 7999 | 0 | A-9 | H | m-D3 | A-9 | m-D3 | H |
| 8000 | 0 | A-10 | H | m-D3 | A-10 | m-D3 | H |
| 8001 | 0 | A-11 | H | m-D3 | A-11 | m-D3 | H |
| 8002 | 0 | A-12 | H | m-D3 | A-12 | m-D3 | H |
| 8003 | 0 | A-13 | H | m-D3 | A-13 | m-D3 | H |
| 8004 | 0 | A-14 | H | m-D3 | A-14 | m-D3 | H |
| 8005 | 0 | A-15 | H | m-D3 | A-15 | m-D3 | H |
| 8006 | 0 | A-16 | H | m-D3 | A-16 | m-D3 | H |
| 8007 | 0 | A-17 | H | m-D3 | A-17 | m-D3 | H |
| 8008 | 0 | A-18 | H | m-D3 | A-18 | m-D3 | H |
| 8009 | 0 | A-19 | H | m-D3 | A-19 | m-D3 | H |
| 8010 | 0 | A-20 | H | m-D3 | A-20 | m-D3 | H |
| 8011 | 0 | A-21 | H | m-D3 | A-21 | m-D3 | H |
| 8012 | 0 | A-22 | H | m-D3 | A-22 | m-D3 | H |
| 8013 | 0 | A-23 | H | m-D3 | A-23 | m-D3 | H |
| 8014 | 0 | A-24 | H | m-D3 | A-24 | m-D3 | H |
| 8015 | 0 | A-25 | H | m-D3 | A-25 | m-D3 | H |
| 8016 | 0 | A-26 | H | m-D3 | A-26 | m-D3 | H |
| 8017 | 0 | A-27 | H | m-D3 | A-27 | m-D3 | H |
| 8018 | 0 | A-28 | H | m-D3 | A-28 | m-D3 | H |
| 8019 | 0 | A-29 | H | m-D3 | A-29 | m-D3 | H |
| 8020 | 0 | A-30 | H | m-D3 | A-30 | m-D3 | H |
| 8021 | 0 | A-31 | H | m-D3 | A-31 | m-D3 | H |
| 8022 | 0 | A-32 | H | m-D3 | A-32 | m-D3 | H |
| 8023 | 0 | A-33 | H | m-D3 | A-33 | m-D3 | H |
| 8024 | 0 | A-34 | H | m-D3 | A-34 | m-D3 | H |
| 8025 | 0 | A-35 | H | m-D3 | A-35 | m-D3 | H |
| 8026 | 0 | A-36 | H | m-D3 | A-36 | m-D3 | H |
| 8027 | 0 | A-37 | H | m-D3 | A-37 | m-D3 | H |
| 8028 | 0 | A-38 | H | m-D3 | A-38 | m-D3 | H |
| 8029 | 0 | A-39 | H | m-D3 | A-39 | m-D3 | H |
| 8030 | 0 | A-40 | H | m-D3 | A-40 | m-D3 | H |
| 8031 | 0 | A-41 | H | m-D3 | A-41 | m-D3 | H |
| 8032 | 0 | A-42 | H | m-D3 | A-42 | m-D3 | H |
| 8033 | 0 | A-43 | H | m-D3 | A-43 | m-D3 | H |
| 8034 | 0 | A-44 | H | m-D3 | A-44 | m-D3 | H |
| 8035 | 0 | A-45 | H | m-D3 | A-45 | m-D3 | H |
| 8036 | 0 | A-46 | H | m-D3 | A-46 | m-D3 | H |
| 8037 | 0 | A-47 | H | m-D3 | A-47 | m-D3 | H |
| 8038 | 0 | A-1 | H | m-D5 | A-1 | m-D5 | H |
| 8039 | 0 | A-2 | H | m-D5 | A-2 | m-D5 | H |
| 8040 | 0 | A-3 | H | m-D5 | A-3 | m-D5 | H |
| 8041 | 0 | A-4 | H | m-D5 | A-4 | m-D5 | H |
| 8042 | 0 | A-5 | H | m-D5 | A-5 | m-D5 | H |
| 8043 | 0 | A-6 | H | m-D5 | A-6 | m-D5 | H |
| 8044 | 0 | A-7 | H | m-D5 | A-7 | m-D5 | H |
| 8045 | 0 | A-8 | H | m-D5 | A-8 | m-D5 | H |
| 8046 | 0 | A-9 | H | m-D5 | A-9 | m-D5 | H |
| 8047 | 0 | A-10 | H | m-D5 | A-10 | m-D5 | H |
| 8048 | 0 | A-11 | H | m-D5 | A-11 | m-D5 | H |
| 8049 | 0 | A-12 | H | m-D5 | A-12 | m-D5 | H |
| 8050 | 0 | A-13 | H | m-D5 | A-13 | m-D5 | H |
| 8051 | 0 | A-14 | H | m-D5 | A-14 | m-D5 | H |
| 8052 | 0 | A-15 | H | m-D5 | A-15 | m-D5 | H |
| 8053 | 0 | A-16 | H | m-D5 | A-16 | m-D5 | H |
| 8054 | 0 | A-17 | H | m-D5 | A-17 | m-D5 | H |
| 8055 | 0 | A-18 | H | m-D5 | A-18 | m-D5 | H |
| 8056 | 0 | A-19 | H | m-D5 | A-19 | m-D5 | H |
| 8057 | 0 | A-20 | H | m-D5 | A-20 | m-D5 | H |
| 8058 | 0 | A-21 | H | m-D5 | A-21 | m-D5 | H |
| 8059 | 0 | A-22 | H | m-D5 | A-22 | m-D5 | H |
| 8060 | 0 | A-23 | H | m-D5 | A-23 | m-D5 | H |
| 8061 | 0 | A-24 | H | m-D5 | A-24 | m-D5 | H |
| 8062 | 0 | A-25 | H | m-D5 | A-25 | m-D5 | H |
| 8063 | 0 | A-26 | H | m-D5 | A-26 | m-D5 | H |
| 8064 | 0 | A-27 | H | m-D5 | A-27 | m-D5 | H |
| 8065 | 0 | A-28 | H | m-D5 | A-28 | m-D5 | H |
| 8066 | 0 | A-29 | H | m-D5 | A-29 | m-D5 | H |
| 8067 | 0 | A-30 | H | m-D5 | A-30 | m-D5 | H |
| 8068 | 0 | A-31 | H | m-D5 | A-31 | m-D5 | H |
| 8069 | 0 | A-32 | H | m-D5 | A-32 | m-D5 | H |
| 8070 | 0 | A-33 | H | m-D5 | A-33 | m-D5 | H |
| 8071 | 0 | A-34 | H | m-D5 | A-34 | m-D5 | H |
| 8072 | 0 | A-35 | H | m-D5 | A-35 | m-D5 | H |
| 8073 | 0 | A-36 | H | m-D5 | A-36 | m-D5 | H |
| 8074 | 0 | A-37 | H | m-D5 | A-37 | m-D5 | H |
| 8075 | 0 | A-38 | H | m-D5 | A-38 | m-D5 | H |
| 8076 | 0 | A-39 | H | m-D5 | A-39 | m-D5 | H |
| 8077 | 0 | A-40 | H | m-D5 | A-40 | m-D5 | H |
| 8078 | 0 | A-41 | H | m-D5 | A-41 | m-D5 | H |
| 8079 | 0 | A-42 | H | m-D5 | A-42 | m-D5 | H |
| 8080 | 0 | A-43 | H | m-D5 | A-43 | m-D5 | H |
| 8081 | 0 | A-44 | H | m-D5 | A-44 | m-D5 | H |
| 8082 | 0 | A-45 | H | m-D5 | A-45 | m-D5 | H |
| 8083 | 0 | A-46 | H | m-D5 | A-46 | m-D5 | H |
| 8084 | 0 | A-47 | H | m-D5 | A-47 | m-D5 | H |
| 8085 | 0 | A-1 | H | m-D9 | A-1 | m-D9 | H |
| 8086 | 0 | A-2 | H | m-D9 | A-2 | m-D9 | H |
| 8087 | 0 | A-3 | H | m-D9 | A-3 | m-D9 | H |
| 8088 | 0 | A-4 | H | m-D9 | A-4 | m-D9 | H |
| 8089 | 0 | A-5 | H | m-D9 | A-5 | m-D9 | H |
| 8090 | 0 | A-6 | H | m-D9 | A-6 | m-D9 | H |
| 8091 | 0 | A-7 | H | m-D9 | A-7 | m-D9 | H |
| 8092 | 0 | A-8 | H | m-D9 | A-8 | m-D9 | H |
| 8093 | 0 | A-9 | H | m-D9 | A-9 | m-D9 | H |
| 8094 | 0 | A-10 | H | m-D9 | A-10 | m-D9 | H |
| 8095 | 0 | A-11 | H | m-D9 | A-11 | m-D9 | H |
| 8096 | 0 | A-12 | H | m-D9 | A-12 | m-D9 | H |
| 8097 | 0 | A-13 | H | m-D9 | A-13 | m-D9 | H |
| 8098 | 0 | A-14 | H | m-D9 | A-14 | m-D9 | H |
| 8099 | 0 | A-15 | H | m-D9 | A-15 | m-D9 | H |
| 8100 | 0 | A-16 | H | m-D9 | A-16 | m-D9 | H |
| 8101 | 0 | A-17 | H | m-D9 | A-17 | m-D9 | H |
| 8102 | 0 | A-18 | H | m-D9 | A-18 | m-D9 | H |
| 8103 | 0 | A-19 | H | m-D9 | A-19 | m-D9 | H |
| 8104 | 0 | A-20 | H | m-D9 | A-20 | m-D9 | H |
| 8105 | 0 | A-21 | H | m-D9 | A-21 | m-D9 | H |
| 8106 | 0 | A-22 | H | m-D9 | A-22 | m-D9 | H |
| 8107 | 0 | A-23 | H | m-D9 | A-23 | m-D9 | H |
| 8108 | 0 | A-24 | H | m-D9 | A-24 | m-D9 | H |
| 8109 | 0 | A-25 | H | m-D9 | A-25 | m-D9 | H |
| 8110 | 0 | A-26 | H | m-D9 | A-26 | m-D9 | H |
| 8111 | 0 | A-27 | H | m-D9 | A-27 | m-D9 | H |
| 8112 | 0 | A-28 | H | m-D9 | A-28 | m-D9 | H |
| 8113 | 0 | A-29 | H | m-D9 | A-29 | m-D9 | H |
| 8114 | 0 | A-30 | H | m-D9 | A-30 | m-D9 | H |
| 8115 | 0 | A-31 | H | m-D9 | A-31 | m-D9 | H |
| 8116 | 0 | A-32 | H | m-D9 | A-32 | m-D9 | H |
| 8117 | 0 | A-33 | H | m-D9 | A-33 | m-D9 | H |
| 8118 | 0 | A-34 | H | m-D9 | A-34 | m-D9 | H |
| 8119 | 0 | A-35 | H | m-D9 | A-35 | m-D9 | H |
| 8120 | 0 | A-36 | H | m-D9 | A-36 | m-D9 | H |
| 8121 | 0 | A-37 | H | m-D9 | A-37 | m-D9 | H |
| 8122 | 0 | A-38 | H | m-D9 | A-38 | m-D9 | H |
| 8123 | 0 | A-39 | H | m-D9 | A-39 | m-D9 | H |
| 8124 | 0 | A-40 | H | m-D9 | A-40 | m-D9 | H |
| 8125 | 0 | A-41 | H | m-D9 | A-41 | m-D9 | H |
| 8126 | 0 | A-42 | H | m-D9 | A-42 | m-D9 | H |
| 8127 | 0 | A-43 | H | m-D9 | A-43 | m-D9 | H |
| 8128 | 0 | A-44 | H | m-D9 | A-44 | m-D9 | H |
| 8129 | 0 | A-45 | H | m-D9 | A-45 | m-D9 | H |
| 8130 | 0 | A-46 | H | m-D9 | A-46 | m-D9 | H |
| 8131 | 0 | A-47 | H | m-D9 | A-47 | m-D9 | H |
| 8132 | 0 | A-1 | H | m-D11 | A-1 | m-D11 | H |
| 8133 | 0 | A-2 | H | m-D11 | A-2 | m-D11 | H |
| 8134 | 0 | A-3 | H | m-D11 | A-3 | m-D11 | H |
| 8135 | 0 | A-4 | H | m-D11 | A-4 | m-D11 | H |
| 8136 | 0 | A-5 | H | m-D11 | A-5 | m-D11 | H |
| 8137 | 0 | A-6 | H | m-D11 | A-6 | m-D11 | H |
| 8138 | 0 | A-7 | H | m-D11 | A-7 | m-D11 | H |
| 8139 | 0 | A-8 | H | m-D11 | A-8 | m-D11 | H |
| 8140 | 0 | A-9 | H | m-D11 | A-9 | m-D11 | H |
| 8141 | 0 | A-10 | H | m-D11 | A-10 | m-D11 | H |
| 8142 | 0 | A-11 | H | m-D11 | A-11 | m-D11 | H |
| 8143 | 0 | A-12 | H | m-D11 | A-12 | m-D11 | H |
| 8144 | 0 | A-13 | H | m-D11 | A-13 | m-D11 | H |
| 8145 | 0 | A-14 | H | m-D11 | A-14 | m-D11 | H |
| 8146 | 0 | A-15 | H | m-D11 | A-15 | m-D11 | H |
| 8147 | 0 | A-16 | H | m-D11 | A-16 | m-D11 | H |
| 8148 | 0 | A-17 | H | m-D11 | A-17 | m-D11 | H |
| 8149 | 0 | A-18 | H | m-D11 | A-18 | m-D11 | H |
| 8150 | 0 | A-19 | H | m-D11 | A-19 | m-D11 | H |
| 8151 | 0 | A-20 | H | m-D11 | A-20 | m-D11 | H |
| 8152 | 0 | A-21 | H | m-D11 | A-21 | m-D11 | H |
| 8153 | 0 | A-22 | H | m-D11 | A-22 | m-D11 | H |
| 8154 | 0 | A-23 | H | m-D11 | A-23 | m-D11 | H |
| 8155 | 0 | A-24 | H | m-D11 | A-24 | m-D11 | H |
| 8156 | 0 | A-25 | H | m-D11 | A-25 | m-D11 | H |
| 8157 | 0 | A-26 | H | m-D11 | A-26 | m-D11 | H |
| 8158 | 0 | A-27 | H | m-D11 | A-27 | m-D11 | H |
| 8159 | 0 | A-28 | H | m-D11 | A-28 | m-D11 | H |
| 8160 | 0 | A-29 | H | m-D11 | A-29 | m-D11 | H |
| 8161 | 0 | A-30 | H | m-D11 | A-30 | m-D11 | H |
| 8162 | 0 | A-31 | H | m-D11 | A-31 | m-D11 | H |
| 8163 | 0 | A-32 | H | m-D11 | A-32 | m-D11 | H |
| 8164 | 0 | A-33 | H | m-D11 | A-33 | m-D11 | H |
| 8165 | 0 | A-34 | H | m-D11 | A-34 | m-D11 | H |
| 8166 | 0 | A-35 | H | m-D11 | A-35 | m-D11 | H |
| 8167 | 0 | A-36 | H | m-D11 | A-36 | m-D11 | H |
| 8168 | 0 | A-37 | H | m-D11 | A-37 | m-D11 | H |
| 8169 | 0 | A-38 | H | m-D11 | A-38 | m-D11 | H |
| 8170 | 0 | A-39 | H | m-D11 | A-39 | m-D11 | H |
| 8171 | 0 | A-40 | H | m-D11 | A-40 | m-D11 | H |
| 8172 | 0 | A-41 | H | m-D11 | A-41 | m-D11 | H |
| 8173 | 0 | A-42 | H | m-D11 | A-42 | m-D11 | H |
| 8174 | 0 | A-43 | H | m-D11 | A-43 | m-D11 | H |
| 8175 | 0 | A-44 | H | m-D11 | A-44 | m-D11 | H |
| 8176 | 0 | A-45 | H | m-D11 | A-45 | m-D11 | H |
| 8177 | 0 | A-46 | H | m-D11 | A-46 | m-D11 | H |
| 8178 | 0 | A-47 | H | m-D11 | A-47 | m-D11 | H |
| 8179 | 0 | A-1 | H | m-D12 | A-1 | m-D12 | H |
| 8180 | 0 | A-2 | H | m-D12 | A-2 | m-D12 | H |
| 8181 | 0 | A-3 | H | m-D12 | A-3 | m-D12 | H |
| 8182 | 0 | A-4 | H | m-D12 | A-4 | m-D12 | H |
| 8183 | 0 | A-5 | H | m-D12 | A-5 | m-D12 | H |
| 8184 | 0 | A-6 | H | m-D12 | A-6 | m-D12 | H |
| 8185 | 0 | A-7 | H | m-D12 | A-7 | m-D12 | H |
| 8186 | 0 | A-8 | H | m-D12 | A-8 | m-D12 | H |
| 8187 | 0 | A-9 | H | m-D12 | A-9 | m-D12 | H |
| 8188 | 0 | A-10 | H | m-D12 | A-10 | m-D12 | H |
| 8189 | 0 | A-11 | H | m-D12 | A-11 | m-D12 | H |
| 8190 | 0 | A-12 | H | m-D12 | A-12 | m-D12 | H |
| 8191 | 0 | A-13 | H | m-D12 | A-13 | m-D12 | H |
| 8192 | 0 | A-14 | H | m-D12 | A-14 | m-D12 | H |
| 8193 | 0 | A-15 | H | m-D12 | A-15 | m-D12 | H |
| 8194 | 0 | A-16 | H | m-D12 | A-16 | m-D12 | H |
| 8195 | 0 | A-17 | H | m-D12 | A-17 | m-D12 | H |
| 8196 | 0 | A-18 | H | m-D12 | A-18 | m-D12 | H |
| 8197 | 0 | A-19 | H | m-D12 | A-19 | m-D12 | H |
| 8198 | 0 | A-20 | H | m-D12 | A-20 | m-D12 | H |
| 8199 | 0 | A-21 | H | m-D12 | A-21 | m-D12 | H |
| 8200 | 0 | A-22 | H | m-D12 | A-22 | m-D12 | H |
| 8201 | 0 | A-23 | H | m-D12 | A-23 | m-D12 | H |
| 8202 | 0 | A-24 | H | m-D12 | A-24 | m-D12 | H |
| 8203 | 0 | A-25 | H | m-D12 | A-25 | m-D12 | H |
| 8204 | 0 | A-26 | H | m-D12 | A-26 | m-D12 | H |
| 8205 | 0 | A-27 | H | m-D12 | A-27 | m-D12 | H |
| 8206 | 0 | A-28 | H | m-D12 | A-28 | m-D12 | H |
| 8207 | 0 | A-29 | H | m-D12 | A-29 | m-D12 | H |
| 8208 | 0 | A-30 | H | m-D12 | A-30 | m-D12 | H |
| 8209 | 0 | A-31 | H | m-D12 | A-31 | m-D12 | H |
| 8210 | 0 | A-32 | H | m-D12 | A-32 | m-D12 | H |
| 8211 | 0 | A-33 | H | m-D12 | A-33 | m-D12 | H |
| 8212 | 0 | A-34 | H | m-D12 | A-34 | m-D12 | H |
| 8213 | 0 | A-35 | H | m-D12 | A-35 | m-D12 | H |
| 8214 | 0 | A-36 | H | m-D12 | A-36 | m-D12 | H |
| 8215 | 0 | A-37 | H | m-D12 | A-37 | m-D12 | H |
| 8216 | 0 | A-38 | H | m-D12 | A-38 | m-D12 | H |
| 8217 | 0 | A-39 | H | m-D12 | A-39 | m-D12 | H |
| 8218 | 0 | A-40 | H | m-D12 | A-40 | m-D12 | H |
| 8219 | 0 | A-41 | H | m-D12 | A-41 | m-D12 | H |
| 8220 | 0 | A-42 | H | m-D12 | A-42 | m-D12 | H |
| 8221 | 0 | A-43 | H | m-D12 | A-43 | m-D12 | H |
| 8222 | 0 | A-44 | H | m-D12 | A-44 | m-D12 | H |
| 8223 | 0 | A-45 | H | m-D12 | A-45 | m-D12 | H |
| 8224 | 0 | A-46 | H | m-D12 | A-46 | m-D12 | H |
| 8225 | 0 | A-47 | H | m-D12 | A-47 | m-D12 | H |
| 8226 | 0 | A-1 | H | m-D13 | A-1 | m-D13 | H |
| 8227 | 0 | A-2 | H | m-D13 | A-2 | m-D13 | H |
| 8228 | 0 | A-3 | H | m-D13 | A-3 | m-D13 | H |
| 8229 | 0 | A-4 | H | m-D13 | A-4 | m-D13 | H |
| 8230 | 0 | A-5 | H | m-D13 | A-5 | m-D13 | H |
| 8231 | 0 | A-6 | H | m-D13 | A-6 | m-D13 | H |
| 8232 | 0 | A-7 | H | m-D13 | A-7 | m-D13 | H |
| 8233 | 0 | A-8 | H | m-D13 | A-8 | m-D13 | H |
| 8234 | 0 | A-9 | H | m-D13 | A-9 | m-D13 | H |
| 8235 | 0 | A-10 | H | m-D13 | A-10 | m-D13 | H |
| 8236 | 0 | A-11 | H | m-D13 | A-11 | m-D13 | H |
| 8237 | 0 | A-12 | H | m-D13 | A-12 | m-D13 | H |
| 8238 | 0 | A-13 | H | m-D13 | A-13 | m-D13 | H |
| 8239 | 0 | A-14 | H | m-D13 | A-14 | m-D13 | H |
| 8240 | 0 | A-15 | H | m-D13 | A-15 | m-D13 | H |
| 8241 | 0 | A-16 | H | m-D13 | A-16 | m-D13 | H |
| 8242 | 0 | A-17 | H | m-D13 | A-17 | m-D13 | H |
| 8243 | 0 | A-18 | H | m-D13 | A-18 | m-D13 | H |
| 8244 | 0 | A-19 | H | m-D13 | A-19 | m-D13 | H |
| 8245 | 0 | A-20 | H | m-D13 | A-20 | m-D13 | H |
| 8246 | 0 | A-21 | H | m-D13 | A-21 | m-D13 | H |
| 8247 | 0 | A-22 | H | m-D13 | A-22 | m-D13 | H |
| 8248 | 0 | A-23 | H | m-D13 | A-23 | m-D13 | H |
| 8249 | 0 | A-24 | H | m-D13 | A-24 | m-D13 | H |
| 8250 | 0 | A-25 | H | m-D13 | A-25 | m-D13 | H |
| 8251 | 0 | A-26 | H | m-D13 | A-26 | m-D13 | H |
| 8252 | 0 | A-27 | H | m-D13 | A-27 | m-D13 | H |
| 8253 | 0 | A-28 | H | m-D13 | A-28 | m-D13 | H |
| 8254 | 0 | A-29 | H | m-D13 | A-29 | m-D13 | H |
| 8255 | 0 | A-30 | H | m-D13 | A-30 | m-D13 | H |
| 8256 | 0 | A-31 | H | m-D13 | A-31 | m-D13 | H |
| 8257 | 0 | A-32 | H | m-D13 | A-32 | m-D13 | H |
| 8258 | 0 | A-33 | H | m-D13 | A-33 | m-D13 | H |
| 8259 | 0 | A-34 | H | m-D13 | A-34 | m-D13 | H |
| 8260 | 0 | A-35 | H | m-D13 | A-35 | m-D13 | H |
| 8261 | 0 | A-36 | H | m-D13 | A-36 | m-D13 | H |
| 8262 | 0 | A-37 | H | m-D13 | A-37 | m-D13 | H |
| 8263 | 0 | A-38 | H | m-D13 | A-38 | m-D13 | H |
| 8264 | 0 | A-39 | H | m-D13 | A-39 | m-D13 | H |
| 8265 | 0 | A-40 | H | m-D13 | A-40 | m-D13 | H |
| 8266 | 0 | A-41 | H | m-D13 | A-41 | m-D13 | H |
| 8267 | 0 | A-42 | H | m-D13 | A-42 | m-D13 | H |
| 8268 | 0 | A-43 | H | m-D13 | A-43 | m-D13 | H |
| 8269 | 0 | A-44 | H | m-D13 | A-44 | m-D13 | H |
| 8270 | 0 | A-45 | H | m-D13 | A-45 | m-D13 | H |
| 8271 | 0 | A-46 | H | m-D13 | A-46 | m-D13 | H |
| 8272 | 0 | A-47 | H | m-D13 | A-47 | m-D13 | H |
| 8273 | 0 | A-1 | H | m-D14 | A-1 | m-D14 | H |
| 8274 | 0 | A-2 | H | m-D14 | A-2 | m-D14 | H |
| 8275 | 0 | A-3 | H | m-D14 | A-3 | m-D14 | H |
| 8276 | 0 | A-4 | H | m-D14 | A-4 | m-D14 | H |
| 8277 | 0 | A-5 | H | m-D14 | A-5 | m-D14 | H |
| 8278 | 0 | A-6 | H | m-D14 | A-6 | m-D14 | H |
| 8279 | 0 | A-7 | H | m-D14 | A-7 | m-D14 | H |
| 8280 | 0 | A-8 | H | m-D14 | A-8 | m-D14 | H |
| 8281 | 0 | A-9 | H | m-D14 | A-9 | m-D14 | H |
| 8282 | 0 | A-10 | H | m-D14 | A-10 | m-D14 | H |
| 8283 | 0 | A-11 | H | m-D14 | A-11 | m-D14 | H |
| 8284 | 0 | A-12 | H | m-D14 | A-12 | m-D14 | H |
| 8285 | 0 | A-13 | H | m-D14 | A-13 | m-D14 | H |
| 8286 | 0 | A-14 | H | m-D14 | A-14 | m-D14 | H |
| 8287 | 0 | A-15 | H | m-D14 | A-15 | m-D14 | H |
| 8288 | 0 | A-16 | H | m-D14 | A-16 | m-D14 | H |
| 8289 | 0 | A-17 | H | m-D14 | A-17 | m-D14 | H |
| 8290 | 0 | A-18 | H | m-D14 | A-18 | m-D14 | H |
| 8291 | 0 | A-19 | H | m-D14 | A-19 | m-D14 | H |
| 8292 | 0 | A-20 | H | m-D14 | A-20 | m-D14 | H |
| 8293 | 0 | A-21 | H | m-D14 | A-21 | m-D14 | H |
| 8294 | 0 | A-22 | H | m-D14 | A-22 | m-D14 | H |
| 8295 | 0 | A-23 | H | m-D14 | A-23 | m-D14 | H |
| 8296 | 0 | A-24 | H | m-D14 | A-24 | m-D14 | H |
| 8297 | 0 | A-25 | H | m-D14 | A-25 | m-D14 | H |
| 8298 | 0 | A-26 | H | m-D14 | A-26 | m-D14 | H |
| 8299 | 0 | A-27 | H | m-D14 | A-27 | m-D14 | H |
| 8300 | 0 | A-28 | H | m-D14 | A-28 | m-D14 | H |
| 8301 | 0 | A-29 | H | m-D14 | A-29 | m-D14 | H |
| 8302 | 0 | A-30 | H | m-D14 | A-30 | m-D14 | H |
| 8303 | 0 | A-31 | H | m-D14 | A-31 | m-D14 | H |
| 8304 | 0 | A-32 | H | m-D14 | A-32 | m-D14 | H |
| 8305 | 0 | A-33 | H | m-D14 | A-33 | m-D14 | H |
| 8306 | 0 | A-34 | H | m-D14 | A-34 | m-D14 | H |
| 8307 | 0 | A-35 | H | m-D14 | A-35 | m-D14 | H |
| 8308 | 0 | A-36 | H | m-D14 | A-36 | m-D14 | H |
| 8309 | 0 | A-37 | H | m-D14 | A-37 | m-D14 | H |
| 8310 | 0 | A-38 | H | m-D14 | A-38 | m-D14 | H |
| 8311 | 0 | A-39 | H | m-D14 | A-39 | m-D14 | H |
| 8312 | 0 | A-40 | H | m-D14 | A-40 | m-D14 | H |
| 8313 | 0 | A-41 | H | m-D14 | A-41 | m-D14 | H |
| 8314 | 0 | A-42 | H | m-D14 | A-42 | m-D14 | H |
| 8315 | 0 | A-43 | H | m-D14 | A-43 | m-D14 | H |
| 8316 | 0 | A-44 | H | m-D14 | A-44 | m-D14 | H |
| 8317 | 0 | A-45 | H | m-D14 | A-45 | m-D14 | H |
| 8318 | 0 | A-46 | H | m-D14 | A-46 | m-D14 | H |
| 8319 | 0 | A-47 | H | m-D14 | A-47 | m-D14 | H |
| 8320 | 0 | m-D1 | H | A-1 | m-D1 | A-1 | H |
| 8321 | 0 | m-D1 | H | A-2 | m-D1 | A-2 | H |
| 8322 | 0 | m-D1 | H | A-3 | m-D1 | A-3 | H |
| 8323 | 0 | m-D1 | H | A-4 | m-D1 | A-4 | H |
| 8324 | 0 | m-D1 | H | A-5 | m-D1 | A-5 | H |
| 8325 | 0 | m-D1 | H | A-6 | m-D1 | A-6 | H |
| 8326 | 0 | m-D1 | H | A-7 | m-D1 | A-7 | H |
| 8327 | 0 | m-D1 | H | A-8 | m-D1 | A-8 | H |
| 8328 | 0 | m-D1 | H | A-9 | m-D1 | A-9 | H |
| 8329 | 0 | m-D1 | H | A-10 | m-D1 | A-10 | H |
| 8330 | 0 | m-D1 | H | A-11 | m-D1 | A-11 | H |
| 8331 | 0 | m-D1 | H | A-12 | m-D1 | A-12 | H |
| 8332 | 0 | m-D1 | H | A-13 | m-D1 | A-13 | H |
| 8333 | 0 | m-D1 | H | A-14 | m-D1 | A-14 | H |
| 8334 | 0 | m-D1 | H | A-15 | m-D1 | A-15 | H |
| 8335 | 0 | m-D1 | H | A-16 | m-D1 | A-16 | H |
| 8336 | 0 | m-D1 | H | A-17 | m-D1 | A-17 | H |
| 8337 | 0 | m-D1 | H | A-18 | m-D1 | A-18 | H |
| 8338 | 0 | m-D1 | H | A-19 | m-D1 | A-19 | H |
| 8339 | 0 | m-D1 | H | A-20 | m-D1 | A-20 | H |
| 8340 | 0 | m-D1 | H | A-21 | m-D1 | A-21 | H |
| 8341 | 0 | m-D1 | H | A-22 | m-D1 | A-22 | H |
| 8342 | 0 | m-D1 | H | A-23 | m-D1 | A-23 | H |
| 8343 | 0 | m-D1 | H | A-24 | m-D1 | A-24 | H |
| 8344 | 0 | m-D1 | H | A-25 | m-D1 | A-25 | H |
| 8345 | 0 | m-D1 | H | A-26 | m-D1 | A-26 | H |
| 8346 | 0 | m-D1 | H | A-27 | m-D1 | A-27 | H |
| 8347 | 0 | m-D1 | H | A-28 | m-D1 | A-28 | H |
| 8348 | 0 | m-D1 | H | A-29 | m-D1 | A-29 | H |
| 8349 | 0 | m-D1 | H | A-30 | m-D1 | A-30 | H |
| 8350 | 0 | m-D1 | H | A-31 | m-D1 | A-31 | H |
| 8351 | 0 | m-D1 | H | A-32 | m-D1 | A-32 | H |
| 8352 | 0 | m-D1 | H | A-33 | m-D1 | A-33 | H |
| 8353 | 0 | m-D1 | H | A-34 | m-D1 | A-34 | H |
| 8354 | 0 | m-D1 | H | A-35 | m-D1 | A-35 | H |
| 8355 | 0 | m-D1 | H | A-36 | m-D1 | A-36 | H |
| 8356 | 0 | m-D1 | H | A-37 | m-D1 | A-37 | H |
| 8357 | 0 | m-D1 | H | A-38 | m-D1 | A-38 | H |
| 8358 | 0 | m-D1 | H | A-39 | m-D1 | A-39 | H |
| 8359 | 0 | m-D1 | H | A-40 | m-D1 | A-40 | H |
| 8360 | 0 | m-D1 | H | A-41 | m-D1 | A-41 | H |
| 8361 | 0 | m-D1 | H | A-42 | m-D1 | A-42 | H |
| 8362 | 0 | m-D1 | H | A-43 | m-D1 | A-43 | H |
| 8363 | 0 | m-D1 | H | A-44 | m-D1 | A-44 | H |
| 8364 | 0 | m-D1 | H | A-45 | m-D1 | A-45 | H |
| 8365 | 0 | m-D1 | H | A-46 | m-D1 | A-46 | H |
| 8366 | 0 | m-D1 | H | A-47 | m-D1 | A-47 | H |
| 8367 | 0 | m-D2 | H | A-1 | m-D2 | A-1 | H |
| 8368 | 0 | m-D2 | H | A-2 | m-D2 | A-2 | H |
| 8369 | 0 | m-D2 | H | A-3 | m-D2 | A-3 | H |
| 8370 | 0 | m-D2 | H | A-4 | m-D2 | A-4 | H |
| 8371 | 0 | m-D2 | H | A-5 | m-D2 | A-5 | H |
| 8372 | 0 | m-D2 | H | A-6 | m-D2 | A-6 | H |
| 8373 | 0 | m-D2 | H | A-7 | m-D2 | A-7 | H |
| 8374 | 0 | m-D2 | H | A-8 | m-D2 | A-8 | H |
| 8375 | 0 | m-D2 | H | A-9 | m-D2 | A-9 | H |
| 8376 | 0 | m-D2 | H | A-10 | m-D2 | A-10 | H |
| 8377 | 0 | m-D2 | H | A-11 | m-D2 | A-11 | H |
| 8378 | 0 | m-D2 | H | A-12 | m-D2 | A-12 | H |
| 8379 | 0 | m-D2 | H | A-13 | m-D2 | A-13 | H |
| 8380 | 0 | m-D2 | H | A-14 | m-D2 | A-14 | H |
| 8381 | 0 | m-D2 | H | A-15 | m-D2 | A-15 | H |
| 8382 | 0 | m-D2 | H | A-16 | m-D2 | A-16 | H |
| 8383 | 0 | m-D2 | H | A-17 | m-D2 | A-17 | H |
| 8384 | 0 | m-D2 | H | A-18 | m-D2 | A-18 | H |
| 8385 | 0 | m-D2 | H | A-19 | m-D2 | A-19 | H |
| 8386 | 0 | m-D2 | H | A-20 | m-D2 | A-20 | H |
| 8387 | 0 | m-D2 | H | A-21 | m-D2 | A-21 | H |
| 8388 | 0 | m-D2 | H | A-22 | m-D2 | A-22 | H |
| 8389 | 0 | m-D2 | H | A-23 | m-D2 | A-23 | H |
| 8390 | 0 | m-D2 | H | A-24 | m-D2 | A-24 | H |
| 8391 | 0 | m-D2 | H | A-25 | m-D2 | A-25 | H |
| 8392 | 0 | m-D2 | H | A-26 | m-D2 | A-26 | H |
| 8393 | 0 | m-D2 | H | A-27 | m-D2 | A-27 | H |
| 8394 | 0 | m-D2 | H | A-28 | m-D2 | A-28 | H |
| 8395 | 0 | m-D2 | H | A-29 | m-D2 | A-29 | H |
| 8396 | 0 | m-D2 | H | A-30 | m-D2 | A-30 | H |
| 8397 | 0 | m-D2 | H | A-31 | m-D2 | A-31 | H |
| 8398 | 0 | m-D2 | H | A-32 | m-D2 | A-32 | H |
| 8399 | 0 | m-D2 | H | A-33 | m-D2 | A-33 | H |
| 8400 | 0 | m-D2 | H | A-34 | m-D2 | A-34 | H |
| 8401 | 0 | m-D2 | H | A-35 | m-D2 | A-35 | H |
| 8402 | 0 | m-D2 | H | A-36 | m-D2 | A-36 | H |
| 8403 | 0 | m-D2 | H | A-37 | m-D2 | A-37 | H |
| 8404 | 0 | m-D2 | H | A-38 | m-D2 | A-38 | H |
| 8405 | 0 | m-D2 | H | A-39 | m-D2 | A-39 | H |
| 8406 | 0 | m-D2 | H | A-40 | m-D2 | A-40 | H |
| 8407 | 0 | m-D2 | H | A-41 | m-D2 | A-41 | H |
| 8408 | 0 | m-D2 | H | A-42 | m-D2 | A-42 | H |
| 8409 | 0 | m-D2 | H | A-43 | m-D2 | A-43 | H |
| 8410 | 0 | m-D2 | H | A-44 | m-D2 | A-44 | H |
| 8411 | 0 | m-D2 | H | A-45 | m-D2 | A-45 | H |
| 8412 | 0 | m-D2 | H | A-46 | m-D2 | A-46 | H |
| 8413 | 0 | m-D2 | H | A-47 | m-D2 | A-47 | H |
| 8414 | 0 | m-D3 | H | A-1 | m-D3 | A-1 | H |
| 8415 | 0 | m-D3 | H | A-2 | m-D3 | A-2 | H |
| 8416 | 0 | m-D3 | H | A-3 | m-D3 | A-3 | H |
| 8417 | 0 | m-D3 | H | A-4 | m-D3 | A-4 | H |
| 8418 | 0 | m-D3 | H | A-5 | m-D3 | A-5 | H |
| 8419 | 0 | m-D3 | H | A-6 | m-D3 | A-6 | H |
| 8420 | 0 | m-D3 | H | A-7 | m-D3 | A-7 | H |
| 8421 | 0 | m-D3 | H | A-8 | m-D3 | A-8 | H |
| 8422 | 0 | m-D3 | H | A-9 | m-D3 | A-9 | H |
| 8423 | 0 | m-D3 | H | A-10 | m-D3 | A-10 | H |
| 8424 | 0 | m-D3 | H | A-11 | m-D3 | A-11 | H |
| 8425 | 0 | m-D3 | H | A-12 | m-D3 | A-12 | H |
| 8426 | 0 | m-D3 | H | A-13 | m-D3 | A-13 | H |
| 8427 | 0 | m-D3 | H | A-14 | m-D3 | A-14 | H |
| 8428 | 0 | m-D3 | H | A-15 | m-D3 | A-15 | H |
| 8429 | 0 | m-D3 | H | A-16 | m-D3 | A-16 | H |
| 8430 | 0 | m-D3 | H | A-17 | m-D3 | A-17 | H |
| 8431 | 0 | m-D3 | H | A-18 | m-D3 | A-18 | H |
| 8432 | 0 | m-D3 | H | A-19 | m-D3 | A-19 | H |
| 8433 | 0 | m-D3 | H | A-20 | m-D3 | A-20 | H |
| 8434 | 0 | m-D3 | H | A-21 | m-D3 | A-21 | H |
| 8435 | 0 | m-D3 | H | A-22 | m-D3 | A-22 | H |
| 8436 | 0 | m-D3 | H | A-23 | m-D3 | A-23 | H |
| 8437 | 0 | m-D3 | H | A-24 | m-D3 | A-24 | H |
| 8438 | 0 | m-D3 | H | A-25 | m-D3 | A-25 | H |
| 8439 | 0 | m-D3 | H | A-26 | m-D3 | A-26 | H |
| 8440 | 0 | m-D3 | H | A-27 | m-D3 | A-27 | H |
| 8441 | 0 | m-D3 | H | A-28 | m-D3 | A-28 | H |
| 8442 | 0 | m-D3 | H | A-29 | m-D3 | A-29 | H |
| 8443 | 0 | m-D3 | H | A-30 | m-D3 | A-30 | H |
| 8444 | 0 | m-D3 | H | A-31 | m-D3 | A-31 | H |
| 8445 | 0 | m-D3 | H | A-32 | m-D3 | A-32 | H |
| 8446 | 0 | m-D3 | H | A-33 | m-D3 | A-33 | H |
| 8447 | 0 | m-D3 | H | A-34 | m-D3 | A-34 | H |
| 8448 | 0 | m-D3 | H | A-35 | m-D3 | A-35 | H |
| 8449 | 0 | m-D3 | H | A-36 | m-D3 | A-36 | H |
| 8450 | 0 | m-D3 | H | A-37 | m-D3 | A-37 | H |
| 8451 | 0 | m-D3 | H | A-38 | m-D3 | A-38 | H |
| 8452 | 0 | m-D3 | H | A-39 | m-D3 | A-39 | H |
| 8453 | 0 | m-D3 | H | A-40 | m-D3 | A-40 | H |
| 8454 | 0 | m-D3 | H | A-41 | m-D3 | A-41 | H |
| 8455 | 0 | m-D3 | H | A-42 | m-D3 | A-42 | H |
| 8456 | 0 | m-D3 | H | A-43 | m-D3 | A-43 | H |
| 8457 | 0 | m-D3 | H | A-44 | m-D3 | A-44 | H |
| 8458 | 0 | m-D3 | H | A-45 | m-D3 | A-45 | H |
| 8459 | 0 | m-D3 | H | A-46 | m-D3 | A-46 | H |
| 8460 | 0 | m-D3 | H | A-47 | m-D3 | A-47 | H |
| 8461 | 0 | m-D5 | H | A-1 | m-D5 | A-1 | H |
| 8462 | 0 | m-D5 | H | A-2 | m-D5 | A-2 | H |
| 8463 | 0 | m-D5 | H | A-3 | m-D5 | A-3 | H |
| 8464 | 0 | m-D5 | H | A-4 | m-D5 | A-4 | H |
| 8465 | 0 | m-D5 | H | A-5 | m-D5 | A-5 | H |
| 8466 | 0 | m-D5 | H | A-6 | m-D5 | A-6 | H |
| 8467 | 0 | m-D5 | H | A-7 | m-D5 | A-7 | H |
| 8468 | 0 | m-D5 | H | A-8 | m-D5 | A-8 | H |
| 8469 | 0 | m-D5 | H | A-9 | m-D5 | A-9 | H |
| 8470 | 0 | m-D5 | H | A-10 | m-D5 | A-10 | H |
| 8471 | 0 | m-D5 | H | A-11 | m-D5 | A-11 | H |
| 8472 | 0 | m-D5 | H | A-12 | m-D5 | A-12 | H |
| 8473 | 0 | m-D5 | H | A-13 | m-D5 | A-13 | H |
| 8474 | 0 | m-D5 | H | A-14 | m-D5 | A-14 | H |
| 8475 | 0 | m-D5 | H | A-15 | m-D5 | A-15 | H |
| 8476 | 0 | m-D5 | H | A-16 | m-D5 | A-16 | H |
| 8477 | 0 | m-D5 | H | A-17 | m-D5 | A-17 | H |
| 8478 | 0 | m-D5 | H | A-18 | m-D5 | A-18 | H |
| 8479 | 0 | m-D5 | H | A-19 | m-D5 | A-19 | H |
| 8480 | 0 | m-D5 | H | A-20 | m-D5 | A-20 | H |
| 8481 | 0 | m-D5 | H | A-21 | m-D5 | A-21 | H |
| 8482 | 0 | m-D5 | H | A-22 | m-D5 | A-22 | H |
| 8483 | 0 | m-D5 | H | A-23 | m-D5 | A-23 | H |
| 8484 | 0 | m-D5 | H | A-24 | m-D5 | A-24 | H |
| 8485 | 0 | m-D5 | H | A-25 | m-D5 | A-25 | H |
| 8486 | 0 | m-D5 | H | A-26 | m-D5 | A-26 | H |
| 8487 | 0 | m-D5 | H | A-27 | m-D5 | A-27 | H |
| 8488 | 0 | m-D5 | H | A-28 | m-D5 | A-28 | H |
| 8489 | 0 | m-D5 | H | A-29 | m-D5 | A-29 | H |
| 8490 | 0 | m-D5 | H | A-30 | m-D5 | A-30 | H |
| 8491 | 0 | m-D5 | H | A-31 | m-D5 | A-31 | H |
| 8492 | 0 | m-D5 | H | A-32 | m-D5 | A-32 | H |
| 8493 | 0 | m-D5 | H | A-33 | m-D5 | A-33 | H |
| 8494 | 0 | m-D5 | H | A-34 | m-D5 | A-34 | H |
| 8495 | 0 | m-D5 | H | A-35 | m-D5 | A-35 | H |
| 8496 | 0 | m-D5 | H | A-36 | m-D5 | A-36 | H |
| 8497 | 0 | m-D5 | H | A-37 | m-D5 | A-37 | H |
| 8498 | 0 | m-D5 | H | A-38 | m-D5 | A-38 | H |
| 8499 | 0 | m-D5 | H | A-39 | m-D5 | A-39 | H |
| 8500 | 0 | m-D5 | H | A-40 | m-D5 | A-40 | H |
| 8501 | 0 | m-D5 | H | A-41 | m-D5 | A-41 | H |
| 8502 | 0 | m-D5 | H | A-42 | m-D5 | A-42 | H |
| 8503 | 0 | m-D5 | H | A-43 | m-D5 | A-43 | H |
| 8504 | 0 | m-D5 | H | A-44 | m-D5 | A-44 | H |
| 8505 | 0 | m-D5 | H | A-45 | m-D5 | A-45 | H |
| 8506 | 0 | m-D5 | H | A-46 | m-D5 | A-46 | H |
| 8507 | 0 | m-D5 | H | A-47 | m-D5 | A-47 | H |
| 8508 | 0 | m-D9 | H | A-1 | m-D9 | A-1 | H |
| 8509 | 0 | m-D9 | H | A-2 | m-D9 | A-2 | H |
| 8510 | 0 | m-D9 | H | A-3 | m-D9 | A-3 | H |
| 8511 | 0 | m-D9 | H | A-4 | m-D9 | A-4 | H |
| 8512 | 0 | m-D9 | H | A-5 | m-D9 | A-5 | H |
| 8513 | 0 | m-D9 | H | A-6 | m-D9 | A-6 | H |
| 8514 | 0 | m-D9 | H | A-7 | m-D9 | A-7 | H |
| 8515 | 0 | m-D9 | H | A-8 | m-D9 | A-8 | H |
| 8516 | 0 | m-D9 | H | A-9 | m-D9 | A-9 | H |
| 8517 | 0 | m-D9 | H | A-10 | m-D9 | A-10 | H |
| 8518 | 0 | m-D9 | H | A-11 | m-D9 | A-11 | H |
| 8519 | 0 | m-D9 | H | A-12 | m-D9 | A-12 | H |
| 8520 | 0 | m-D9 | H | A-13 | m-D9 | A-13 | H |
| 8521 | 0 | m-D9 | H | A-14 | m-D9 | A-14 | H |
| 8522 | 0 | m-D9 | H | A-15 | m-D9 | A-15 | H |
| 8523 | 0 | m-D9 | H | A-16 | m-D9 | A-16 | H |
| 8524 | 0 | m-D9 | H | A-17 | m-D9 | A-17 | H |
| 8525 | 0 | m-D9 | H | A-18 | m-D9 | A-18 | H |
| 8526 | 0 | m-D9 | H | A-19 | m-D9 | A-19 | H |
| 8527 | 0 | m-D9 | H | A-20 | m-D9 | A-20 | H |
| 8528 | 0 | m-D9 | H | A-21 | m-D9 | A-21 | H |
| 8529 | 0 | m-D9 | H | A-22 | m-D9 | A-22 | H |
| 8530 | 0 | m-D9 | H | A-23 | m-D9 | A-23 | H |
| 8531 | 0 | m-D9 | H | A-24 | m-D9 | A-24 | H |
| 8532 | 0 | m-D9 | H | A-25 | m-D9 | A-25 | H |
| 8533 | 0 | m-D9 | H | A-26 | m-D9 | A-26 | H |
| 8534 | 0 | m-D9 | H | A-27 | m-D9 | A-27 | H |
| 8535 | 0 | m-D9 | H | A-28 | m-D9 | A-28 | H |
| 8536 | 0 | m-D9 | H | A-29 | m-D9 | A-29 | H |
| 8537 | 0 | m-D9 | H | A-30 | m-D9 | A-30 | H |
| 8538 | 0 | m-D9 | H | A-31 | m-D9 | A-31 | H |
| 8539 | 0 | m-D9 | H | A-32 | m-D9 | A-32 | H |
| 8540 | 0 | m-D9 | H | A-33 | m-D9 | A-33 | H |
| 8541 | 0 | m-D9 | H | A-34 | m-D9 | A-34 | H |
| 8542 | 0 | m-D9 | H | A-35 | m-D9 | A-35 | H |
| 8543 | 0 | m-D9 | H | A-36 | m-D9 | A-36 | H |
| 8544 | 0 | m-D9 | H | A-37 | m-D9 | A-37 | H |
| 8545 | 0 | m-D9 | H | A-38 | m-D9 | A-38 | H |
| 8546 | 0 | m-D9 | H | A-39 | m-D9 | A-39 | H |
| 8547 | 0 | m-D9 | H | A-40 | m-D9 | A-40 | H |
| 8548 | 0 | m-D9 | H | A-41 | m-D9 | A-41 | H |
| 8549 | 0 | m-D9 | H | A-42 | m-D9 | A-42 | H |
| 8550 | 0 | m-D9 | H | A-43 | m-D9 | A-43 | H |
| 8551 | 0 | m-D9 | H | A-44 | m-D9 | A-44 | H |
| 8552 | 0 | m-D9 | H | A-45 | m-D9 | A-45 | H |
| 8553 | 0 | m-D9 | H | A-46 | m-D9 | A-46 | H |
| 8554 | 0 | m-D9 | H | A-47 | m-D9 | A-47 | H |
| 8555 | 0 | m-D11 | H | A-1 | m-D11 | A-1 | H |
| 8556 | 0 | m-D11 | H | A-2 | m-D11 | A-2 | H |
| 8557 | 0 | m-D11 | H | A-3 | m-D11 | A-3 | H |
| 8558 | 0 | m-D11 | H | A-4 | m-D11 | A-4 | H |
| 8559 | 0 | m-D11 | H | A-5 | m-D11 | A-5 | H |
| 8560 | 0 | m-D11 | H | A-6 | m-D11 | A-6 | H |
| 8561 | 0 | m-D11 | H | A-7 | m-D11 | A-7 | H |
| 8562 | 0 | m-D11 | H | A-8 | m-D11 | A-8 | H |
| 8563 | 0 | m-D11 | H | A-9 | m-D11 | A-9 | H |
| 8564 | 0 | m-D11 | H | A-10 | m-D11 | A-10 | H |
| 8565 | 0 | m-D11 | H | A-11 | m-D11 | A-11 | H |
| 8566 | 0 | m-D11 | H | A-12 | m-D11 | A-12 | H |
| 8567 | 0 | m-D11 | H | A-13 | m-D11 | A-13 | H |
| 8568 | 0 | m-D11 | H | A-14 | m-D11 | A-14 | H |
| 8569 | 0 | m-D11 | H | A-15 | m-D11 | A-15 | H |
| 8570 | 0 | m-D11 | H | A-16 | m-D11 | A-16 | H |
| 8571 | 0 | m-D11 | H | A-17 | m-D11 | A-17 | H |
| 8572 | 0 | m-D11 | H | A-18 | m-D11 | A-18 | H |
| 8573 | 0 | m-D11 | H | A-19 | m-D11 | A-19 | H |
| 8574 | 0 | m-D11 | H | A-20 | m-D11 | A-20 | H |
| 8575 | 0 | m-D11 | H | A-21 | m-D11 | A-21 | H |
| 8576 | 0 | m-D11 | H | A-22 | m-D11 | A-22 | H |
| 8577 | 0 | m-D11 | H | A-23 | m-D11 | A-23 | H |
| 8578 | 0 | m-D11 | H | A-24 | m-D11 | A-24 | H |
| 8579 | 0 | m-D11 | H | A-25 | m-D11 | A-25 | H |
| 8580 | 0 | m-D11 | H | A-26 | m-D11 | A-26 | H |
| 8581 | 0 | m-D11 | H | A-27 | m-D11 | A-27 | H |
| 8582 | 0 | m-D11 | H | A-28 | m-D11 | A-28 | H |
| 8583 | 0 | m-D11 | H | A-29 | m-D11 | A-29 | H |
| 8584 | 0 | m-D11 | H | A-30 | m-D11 | A-30 | H |
| 8585 | 0 | m-D11 | H | A-31 | m-D11 | A-31 | H |
| 8586 | 0 | m-D11 | H | A-32 | m-D11 | A-32 | H |
| 8587 | 0 | m-D11 | H | A-33 | m-D11 | A-33 | H |
| 8588 | 0 | m-D11 | H | A-34 | m-D11 | A-34 | H |
| 8589 | 0 | m-D11 | H | A-35 | m-D11 | A-35 | H |
| 8590 | 0 | m-D11 | H | A-36 | m-D11 | A-36 | H |
| 8591 | 0 | m-D11 | H | A-37 | m-D11 | A-37 | H |
| 8592 | 0 | m-D11 | H | A-38 | m-D11 | A-38 | H |
| 8593 | 0 | m-D11 | H | A-39 | m-D11 | A-39 | H |
| 8594 | 0 | m-D11 | H | A-40 | m-D11 | A-40 | H |
| 8595 | 0 | m-D11 | H | A-41 | m-D11 | A-41 | H |
| 8596 | 0 | m-D11 | H | A-42 | m-D11 | A-42 | H |
| 8597 | 0 | m-D11 | H | A-43 | m-D11 | A-43 | H |
| 8598 | 0 | m-D11 | H | A-44 | m-D11 | A-44 | H |
| 8599 | 0 | m-D11 | H | A-45 | m-D11 | A-45 | H |
| 8600 | 0 | m-D11 | H | A-46 | m-D11 | A-46 | H |
| 8601 | 0 | m-D11 | H | A-47 | m-D11 | A-47 | H |
| 8602 | 0 | m-D12 | H | A-1 | m-D12 | A-1 | H |
| 8603 | 0 | m-D12 | H | A-2 | m-D12 | A-2 | H |
| 8604 | 0 | m-D12 | H | A-3 | m-D12 | A-3 | H |
| 8605 | 0 | m-D12 | H | A-4 | m-D12 | A-4 | H |
| 8606 | 0 | m-D12 | H | A-5 | m-D12 | A-5 | H |
| 8607 | 0 | m-D12 | H | A-6 | m-D12 | A-6 | H |
| 8608 | 0 | m-D12 | H | A-7 | m-D12 | A-7 | H |
| 8609 | 0 | m-D12 | H | A-8 | m-D12 | A-8 | H |
| 8610 | 0 | m-D12 | H | A-9 | m-D12 | A-9 | H |
| 8611 | 0 | m-D12 | H | A-10 | m-D12 | A-10 | H |
| 8612 | 0 | m-D12 | H | A-11 | m-D12 | A-11 | H |
| 8613 | 0 | m-D12 | H | A-12 | m-D12 | A-12 | H |
| 8614 | 0 | m-D12 | H | A-13 | m-D12 | A-13 | H |
| 8615 | 0 | m-D12 | H | A-14 | m-D12 | A-14 | H |
| 8616 | 0 | m-D12 | H | A-15 | m-D12 | A-15 | H |
| 8617 | 0 | m-D12 | H | A-16 | m-D12 | A-16 | H |
| 8618 | 0 | m-D12 | H | A-17 | m-D12 | A-17 | H |
| 8619 | 0 | m-D12 | H | A-18 | m-D12 | A-18 | H |
| 8620 | 0 | m-D12 | H | A-19 | m-D12 | A-19 | H |
| 8621 | 0 | m-D12 | H | A-20 | m-D12 | A-20 | H |
| 8622 | 0 | m-D12 | H | A-21 | m-D12 | A-21 | H |
| 8623 | 0 | m-D12 | H | A-22 | m-D12 | A-22 | H |
| 8624 | 0 | m-D12 | H | A-23 | m-D12 | A-23 | H |
| 8625 | 0 | m-D12 | H | A-24 | m-D12 | A-24 | H |
| 8626 | 0 | m-D12 | H | A-25 | m-D12 | A-25 | H |
| 8627 | 0 | m-D12 | H | A-26 | m-D12 | A-26 | H |
| 8628 | 0 | m-D12 | H | A-27 | m-D12 | A-27 | H |
| 8629 | 0 | m-D12 | H | A-28 | m-D12 | A-28 | H |
| 8630 | 0 | m-D12 | H | A-29 | m-D12 | A-29 | H |
| 8631 | 0 | m-D12 | H | A-30 | m-D12 | A-30 | H |
| 8632 | 0 | m-D12 | H | A-31 | m-D12 | A-31 | H |
| 8633 | 0 | m-D12 | H | A-32 | m-D12 | A-32 | H |
| 8634 | 0 | m-D12 | H | A-33 | m-D12 | A-33 | H |
| 8635 | 0 | m-D12 | H | A-34 | m-D12 | A-34 | H |
| 8636 | 0 | m-D12 | H | A-35 | m-D12 | A-35 | H |
| 8637 | 0 | m-D12 | H | A-36 | m-D12 | A-36 | H |
| 8638 | 0 | m-D12 | H | A-37 | m-D12 | A-37 | H |
| 8639 | 0 | m-D12 | H | A-38 | m-D12 | A-38 | H |
| 8640 | 0 | m-D12 | H | A-39 | m-D12 | A-39 | H |
| 8641 | 0 | m-D12 | H | A-40 | m-D12 | A-40 | H |
| 8642 | 0 | m-D12 | H | A-41 | m-D12 | A-41 | H |
| 8643 | 0 | m-D12 | H | A-42 | m-D12 | A-42 | H |
| 8644 | 0 | m-D12 | H | A-43 | m-D12 | A-43 | H |
| 8645 | 0 | m-D12 | H | A-44 | m-D12 | A-44 | H |
| 8646 | 0 | m-D12 | H | A-45 | m-D12 | A-45 | H |
| 8647 | 0 | m-D12 | H | A-46 | m-D12 | A-46 | H |
| 8648 | 0 | m-D12 | H | A-47 | m-D12 | A-47 | H |
| 8649 | 0 | m-D13 | H | A-1 | m-D13 | A-1 | H |
| 8650 | 0 | m-D13 | H | A-2 | m-D13 | A-2 | H |
| 8651 | 0 | m-D13 | H | A-3 | m-D13 | A-3 | H |
| 8652 | 0 | m-D13 | H | A-4 | m-D13 | A-4 | H |
| 8653 | 0 | m-D13 | H | A-5 | m-D13 | A-5 | H |
| 8654 | 0 | m-D13 | H | A-6 | m-D13 | A-6 | H |
| 8655 | 0 | m-D13 | H | A-7 | m-D13 | A-7 | H |
| 8656 | 0 | m-D13 | H | A-8 | m-D13 | A-8 | H |
| 8657 | 0 | m-D13 | H | A-9 | m-D13 | A-9 | H |
| 8658 | 0 | m-D13 | H | A-10 | m-D13 | A-10 | H |
| 8659 | 0 | m-D13 | H | A-11 | m-D13 | A-11 | H |
| 8660 | 0 | m-D13 | H | A-12 | m-D13 | A-12 | H |
| 8661 | 0 | m-D13 | H | A-13 | m-D13 | A-13 | H |
| 8662 | 0 | m-D13 | H | A-14 | m-D13 | A-14 | H |
| 8663 | 0 | m-D13 | H | A-15 | m-D13 | A-15 | H |
| 8664 | 0 | m-D13 | H | A-16 | m-D13 | A-16 | H |
| 8665 | 0 | m-D13 | H | A-17 | m-D13 | A-17 | H |
| 8666 | 0 | m-D13 | H | A-18 | m-D13 | A-18 | H |
| 8667 | 0 | m-D13 | H | A-19 | m-D13 | A-19 | H |
| 8668 | 0 | m-D13 | H | A-20 | m-D13 | A-20 | H |
| 8669 | 0 | m-D13 | H | A-21 | m-D13 | A-21 | H |
| 8670 | 0 | m-D13 | H | A-22 | m-D13 | A-22 | H |
| 8671 | 0 | m-D13 | H | A-23 | m-D13 | A-23 | H |
| 8672 | 0 | m-D13 | H | A-24 | m-D13 | A-24 | H |
| 8673 | 0 | m-D13 | H | A-25 | m-D13 | A-25 | H |
| 8674 | 0 | m-D13 | H | A-26 | m-D13 | A-26 | H |
| 8675 | 0 | m-D13 | H | A-27 | m-D13 | A-27 | H |
| 8676 | 0 | m-D13 | H | A-28 | m-D13 | A-28 | H |
| 8677 | 0 | m-D13 | H | A-29 | m-D13 | A-29 | H |
| 8678 | 0 | m-D13 | H | A-30 | m-D13 | A-30 | H |
| 8679 | 0 | m-D13 | H | A-31 | m-D13 | A-31 | H |
| 8680 | 0 | m-D13 | H | A-32 | m-D13 | A-32 | H |
| 8681 | 0 | m-D13 | H | A-33 | m-D13 | A-33 | H |
| 8682 | 0 | m-D13 | H | A-34 | m-D13 | A-34 | H |
| 8683 | 0 | m-D13 | H | A-35 | m-D13 | A-35 | H |
| 8684 | 0 | m-D13 | H | A-36 | m-D13 | A-36 | H |
| 8685 | 0 | m-D13 | H | A-37 | m-D13 | A-37 | H |
| 8686 | 0 | m-D13 | H | A-38 | m-D13 | A-38 | H |
| 8687 | 0 | m-D13 | H | A-39 | m-D13 | A-39 | H |
| 8688 | 0 | m-D13 | H | A-40 | m-D13 | A-40 | H |
| 8689 | 0 | m-D13 | H | A-41 | m-D13 | A-41 | H |
| 8690 | 0 | m-D13 | H | A-42 | m-D13 | A-42 | H |
| 8691 | 0 | m-D13 | H | A-43 | m-D13 | A-43 | H |
| 8692 | 0 | m-D13 | H | A-44 | m-D13 | A-44 | H |
| 8693 | 0 | m-D13 | H | A-45 | m-D13 | A-45 | H |
| 8694 | 0 | m-D13 | H | A-46 | m-D13 | A-46 | H |
| 8695 | 0 | m-D13 | H | A-47 | m-D13 | A-47 | H |
| 8696 | 0 | m-D14 | H | A-1 | m-D14 | A-1 | H |
| 8697 | 0 | m-D14 | H | A-2 | m-D14 | A-2 | H |
| 8698 | 0 | m-D14 | H | A-3 | m-D14 | A-3 | H |
| 8699 | 0 | m-D14 | H | A-4 | m-D14 | A-4 | H |
| 8700 | 0 | m-D14 | H | A-5 | m-D14 | A-5 | H |
| 8701 | 0 | m-D14 | H | A-6 | m-D14 | A-6 | H |
| 8702 | 0 | m-D14 | H | A-7 | m-D14 | A-7 | H |
| 8703 | 0 | m-D14 | H | A-8 | m-D14 | A-8 | H |
| 8704 | 0 | m-D14 | H | A-9 | m-D14 | A-9 | H |
| 8705 | 0 | m-D14 | H | A-10 | m-D14 | A-10 | H |
| 8706 | 0 | m-D14 | H | A-11 | m-D14 | A-11 | H |
| 8707 | 0 | m-D14 | H | A-12 | m-D14 | A-12 | H |
| 8708 | 0 | m-D14 | H | A-13 | m-D14 | A-13 | H |
| 8709 | 0 | m-D14 | H | A-14 | m-D14 | A-14 | H |
| 8710 | 0 | m-D14 | H | A-15 | m-D14 | A-15 | H |
| 8711 | 0 | m-D14 | H | A-16 | m-D14 | A-16 | H |
| 8712 | 0 | m-D14 | H | A-17 | m-D14 | A-17 | H |
| 8713 | 0 | m-D14 | H | A-18 | m-D14 | A-18 | H |
| 8714 | 0 | m-D14 | H | A-19 | m-D14 | A-19 | H |
| 8715 | 0 | m-D14 | H | A-20 | m-D14 | A-20 | H |
| 8716 | 0 | m-D14 | H | A-21 | m-D14 | A-21 | H |
| 8717 | 0 | m-D14 | H | A-22 | m-D14 | A-22 | H |
| 8718 | 0 | m-D14 | H | A-23 | m-D14 | A-23 | H |
| 8719 | 0 | m-D14 | H | A-24 | m-D14 | A-24 | H |
| 8720 | 0 | m-D14 | H | A-25 | m-D14 | A-25 | H |
| 8721 | 0 | m-D14 | H | A-26 | m-D14 | A-26 | H |
| 8722 | 0 | m-D14 | H | A-27 | m-D14 | A-27 | H |
| 8723 | 0 | m-D14 | H | A-28 | m-D14 | A-28 | H |
| 8724 | 0 | m-D14 | H | A-29 | m-D14 | A-29 | H |
| 8725 | 0 | m-D14 | H | A-30 | m-D14 | A-30 | H |
| 8726 | 0 | m-D14 | H | A-31 | m-D14 | A-31 | H |
| 8727 | 0 | m-D14 | H | A-32 | m-D14 | A-32 | H |
| 8728 | 0 | m-D14 | H | A-33 | m-D14 | A-33 | H |
| 8729 | 0 | m-D14 | H | A-34 | m-D14 | A-34 | H |
| 8730 | 0 | m-D14 | H | A-35 | m-D14 | A-35 | H |
| 8731 | 0 | m-D14 | H | A-36 | m-D14 | A-36 | H |
| 8732 | 0 | m-D14 | H | A-37 | m-D14 | A-37 | H |
| 8733 | 0 | m-D14 | H | A-38 | m-D14 | A-38 | H |
| 8734 | 0 | m-D14 | H | A-39 | m-D14 | A-39 | H |
| 8735 | 0 | m-D14 | H | A-40 | m-D14 | A-40 | H |
| 8736 | 0 | m-D14 | H | A-41 | m-D14 | A-41 | H |
| 8737 | 0 | m-D14 | H | A-42 | m-D14 | A-42 | H |
| 8738 | 0 | m-D14 | H | A-43 | m-D14 | A-43 | H |
| 8739 | 0 | m-D14 | H | A-44 | m-D14 | A-44 | H |
| 8740 | 0 | m-D14 | H | A-45 | m-D14 | A-45 | H |
| 8741 | 0 | m-D14 | H | A-46 | m-D14 | A-46 | H |
| 8742 | 0 | m-D14 | H | A-47 | m-D14 | A-47 | H |
| 8743 | 0 | m-D1 | A-1 | H | m-D1 | A-1 | H |
| 8744 | 0 | m-D1 | A-2 | H | m-D1 | A-2 | H |
| 8745 | 0 | m-D1 | A-3 | H | m-D1 | A-3 | H |
| 8746 | 0 | m-D1 | A-4 | H | m-D1 | A-4 | H |
| 8747 | 0 | m-D1 | A-5 | H | m-D1 | A-5 | H |
| 8748 | 0 | m-D1 | A-6 | H | m-D1 | A-6 | H |
| 8749 | 0 | m-D1 | A-7 | H | m-D1 | A-7 | H |
| 8750 | 0 | m-D1 | A-8 | H | m-D1 | A-8 | H |
| 8751 | 0 | m-D1 | A-9 | H | m-D1 | A-9 | H |
| 8752 | 0 | m-D1 | A-10 | H | m-D1 | A-10 | H |
| 8753 | 0 | m-D1 | A-11 | H | m-D1 | A-11 | H |
| 8754 | 0 | m-D1 | A-12 | H | m-D1 | A-12 | H |
| 8755 | 0 | m-D1 | A-13 | H | m-D1 | A-13 | H |
| 8756 | 0 | m-D1 | A-14 | H | m-D1 | A-14 | H |
| 8757 | 0 | m-D1 | A-15 | H | m-D1 | A-15 | H |
| 8758 | 0 | m-D1 | A-16 | H | m-D1 | A-16 | H |
| 8759 | 0 | m-D1 | A-17 | H | m-D1 | A-17 | H |
| 8760 | 0 | m-D1 | A-18 | H | m-D1 | A-18 | H |
| 8761 | 0 | m-D1 | A-19 | H | m-D1 | A-19 | H |
| 8762 | 0 | m-D1 | A-20 | H | m-D1 | A-20 | H |
| 8763 | 0 | m-D1 | A-21 | H | m-D1 | A-21 | H |
| 8764 | 0 | m-D1 | A-22 | H | m-D1 | A-22 | H |
| 8765 | 0 | m-D1 | A-23 | H | m-D1 | A-23 | H |
| 8766 | 0 | m-D1 | A-24 | H | m-D1 | A-24 | H |
| 8767 | 0 | m-D1 | A-25 | H | m-D1 | A-25 | H |
| 8768 | 0 | m-D1 | A-26 | H | m-D1 | A-26 | H |
| 8769 | 0 | m-D1 | A-27 | H | m-D1 | A-27 | H |
| 8770 | 0 | m-D1 | A-28 | H | m-D1 | A-28 | H |
| 8771 | 0 | m-D1 | A-29 | H | m-D1 | A-29 | H |
| 8772 | 0 | m-D1 | A-30 | H | m-D1 | A-30 | H |
| 8773 | 0 | m-D1 | A-31 | H | m-D1 | A-31 | H |
| 8774 | 0 | m-D1 | A-32 | H | m-D1 | A-32 | H |
| 8775 | 0 | m-D1 | A-33 | H | m-D1 | A-33 | H |
| 8776 | 0 | m-D1 | A-34 | H | m-D1 | A-34 | H |
| 8777 | 0 | m-D1 | A-35 | H | m-D1 | A-35 | H |
| 8778 | 0 | m-D1 | A-36 | H | m-D1 | A-36 | H |
| 8779 | 0 | m-D1 | A-37 | H | m-D1 | A-37 | H |
| 8780 | 0 | m-D1 | A-38 | H | m-D1 | A-38 | H |
| 8781 | 0 | m-D1 | A-39 | H | m-D1 | A-39 | H |
| 8782 | 0 | m-D1 | A-40 | H | m-D1 | A-40 | H |
| 8783 | 0 | m-D1 | A-41 | H | m-D1 | A-41 | H |
| 8784 | 0 | m-D1 | A-42 | H | m-D1 | A-42 | H |
| 8785 | 0 | m-D1 | A-43 | H | m-D1 | A-43 | H |
| 8786 | 0 | m-D1 | A-44 | H | m-D1 | A-44 | H |
| 8787 | 0 | m-D1 | A-45 | H | m-D1 | A-45 | H |
| 8788 | 0 | m-D1 | A-46 | H | m-D1 | A-46 | H |
| 8789 | 0 | m-D1 | A-47 | H | m-D1 | A-47 | H |
| 8790 | 0 | m-D2 | A-1 | H | m-D2 | A-1 | H |
| 8791 | 0 | m-D2 | A-2 | H | m-D2 | A-2 | H |
| 8792 | 0 | m-D2 | A-3 | H | m-D2 | A-3 | H |
| 8793 | 0 | m-D2 | A-4 | H | m-D2 | A-4 | H |
| 8794 | 0 | m-D2 | A-5 | H | m-D2 | A-5 | H |
| 8795 | 0 | m-D2 | A-6 | H | m-D2 | A-6 | H |
| 8796 | 0 | m-D2 | A-7 | H | m-D2 | A-7 | H |
| 8797 | 0 | m-D2 | A-8 | H | m-D2 | A-8 | H |
| 8798 | 0 | m-D2 | A-9 | H | m-D2 | A-9 | H |
| 8799 | 0 | m-D2 | A-10 | H | m-D2 | A-10 | H |
| 8800 | 0 | m-D2 | A-11 | H | m-D2 | A-11 | H |
| 8801 | 0 | m-D2 | A-12 | H | m-D2 | A-12 | H |
| 8802 | 0 | m-D2 | A-13 | H | m-D2 | A-13 | H |
| 8803 | 0 | m-D2 | A-14 | H | m-D2 | A-14 | H |
| 8804 | 0 | m-D2 | A-15 | H | m-D2 | A-15 | H |
| 8805 | 0 | m-D2 | A-16 | H | m-D2 | A-16 | H |
| 8806 | 0 | m-D2 | A-17 | H | m-D2 | A-17 | H |
| 8807 | 0 | m-D2 | A-18 | H | m-D2 | A-18 | H |
| 8808 | 0 | m-D2 | A-19 | H | m-D2 | A-19 | H |
| 8809 | 0 | m-D2 | A-20 | H | m-D2 | A-20 | H |
| 8810 | 0 | m-D2 | A-21 | H | m-D2 | A-21 | H |
| 8811 | 0 | m-D2 | A-22 | H | m-D2 | A-22 | H |
| 8812 | 0 | m-D2 | A-23 | H | m-D2 | A-23 | H |
| 8813 | 0 | m-D2 | A-24 | H | m-D2 | A-24 | H |
| 8814 | 0 | m-D2 | A-25 | H | m-D2 | A-25 | H |
| 8815 | 0 | m-D2 | A-26 | H | m-D2 | A-26 | H |
| 8816 | 0 | m-D2 | A-27 | H | m-D2 | A-27 | H |
| 8817 | 0 | m-D2 | A-28 | H | m-D2 | A-28 | H |
| 8818 | 0 | m-D2 | A-29 | H | m-D2 | A-29 | H |
| 8819 | 0 | m-D2 | A-30 | H | m-D2 | A-30 | H |
| 8820 | 0 | m-D2 | A-31 | H | m-D2 | A-31 | H |
| 8821 | 0 | m-D2 | A-32 | H | m-D2 | A-32 | H |
| 8822 | 0 | m-D2 | A-33 | H | m-D2 | A-33 | H |
| 8823 | 0 | m-D2 | A-34 | H | m-D2 | A-34 | H |
| 8824 | 0 | m-D2 | A-35 | H | m-D2 | A-35 | H |
| 8825 | 0 | m-D2 | A-36 | H | m-D2 | A-36 | H |
| 8826 | 0 | m-D2 | A-37 | H | m-D2 | A-37 | H |
| 8827 | 0 | m-D2 | A-38 | H | m-D2 | A-38 | H |
| 8828 | 0 | m-D2 | A-39 | H | m-D2 | A-39 | H |
| 8829 | 0 | m-D2 | A-40 | H | m-D2 | A-40 | H |
| 8830 | 0 | m-D2 | A-41 | H | m-D2 | A-41 | H |
| 8831 | 0 | m-D2 | A-42 | H | m-D2 | A-42 | H |
| 8832 | 0 | m-D2 | A-43 | H | m-D2 | A-43 | H |
| 8833 | 0 | m-D2 | A-44 | H | m-D2 | A-44 | H |
| 8834 | 0 | m-D2 | A-45 | H | m-D2 | A-45 | H |
| 8835 | 0 | m-D2 | A-46 | H | m-D2 | A-46 | H |
| 8836 | 0 | m-D2 | A-47 | H | m-D2 | A-47 | H |
| 8837 | 0 | m-D3 | A-1 | H | m-D3 | A-1 | H |
| 8838 | 0 | m-D3 | A-2 | H | m-D3 | A-2 | H |
| 8839 | 0 | m-D3 | A-3 | H | m-D3 | A-3 | H |
| 8840 | 0 | m-D3 | A-4 | H | m-D3 | A-4 | H |
| 8841 | 0 | m-D3 | A-5 | H | m-D3 | A-5 | H |
| 8842 | 0 | m-D3 | A-6 | H | m-D3 | A-6 | H |
| 8843 | 0 | m-D3 | A-7 | H | m-D3 | A-7 | H |
| 8844 | 0 | m-D3 | A-8 | H | m-D3 | A-8 | H |
| 8845 | 0 | m-D3 | A-9 | H | m-D3 | A-9 | H |
| 8846 | 0 | m-D3 | A-10 | H | m-D3 | A-10 | H |
| 8847 | 0 | m-D3 | A-11 | H | m-D3 | A-11 | H |
| 8848 | 0 | m-D3 | A-12 | H | m-D3 | A-12 | H |
| 8849 | 0 | m-D3 | A-13 | H | m-D3 | A-13 | H |
| 8850 | 0 | m-D3 | A-14 | H | m-D3 | A-14 | H |
| 8851 | 0 | m-D3 | A-15 | H | m-D3 | A-15 | H |
| 8852 | 0 | m-D3 | A-16 | H | m-D3 | A-16 | H |
| 8853 | 0 | m-D3 | A-17 | H | m-D3 | A-17 | H |
| 8854 | 0 | m-D3 | A-18 | H | m-D3 | A-18 | H |
| 8855 | 0 | m-D3 | A-19 | H | m-D3 | A-19 | H |
| 8856 | 0 | m-D3 | A-20 | H | m-D3 | A-20 | H |
| 8857 | 0 | m-D3 | A-21 | H | m-D3 | A-21 | H |
| 8858 | 0 | m-D3 | A-22 | H | m-D3 | A-22 | H |
| 8859 | 0 | m-D3 | A-23 | H | m-D3 | A-23 | H |
| 8860 | 0 | m-D3 | A-24 | H | m-D3 | A-24 | H |
| 8861 | 0 | m-D3 | A-25 | H | m-D3 | A-25 | H |
| 8862 | 0 | m-D3 | A-26 | H | m-D3 | A-26 | H |
| 8863 | 0 | m-D3 | A-27 | H | m-D3 | A-27 | H |
| 8864 | 0 | m-D3 | A-28 | H | m-D3 | A-28 | H |
| 8865 | 0 | m-D3 | A-29 | H | m-D3 | A-29 | H |
| 8866 | 0 | m-D3 | A-30 | H | m-D3 | A-30 | H |
| 8867 | 0 | m-D3 | A-31 | H | m-D3 | A-31 | H |
| 8868 | 0 | m-D3 | A-32 | H | m-D3 | A-32 | H |
| 8869 | 0 | m-D3 | A-33 | H | m-D3 | A-33 | H |
| 8870 | 0 | m-D3 | A-34 | H | m-D3 | A-34 | H |
| 8871 | 0 | m-D3 | A-35 | H | m-D3 | A-35 | H |
| 8872 | 0 | m-D3 | A-36 | H | m-D3 | A-36 | H |
| 8873 | 0 | m-D3 | A-37 | H | m-D3 | A-37 | H |
| 8874 | 0 | m-D3 | A-38 | H | m-D3 | A-38 | H |
| 8875 | 0 | m-D3 | A-39 | H | m-D3 | A-39 | H |
| 8876 | 0 | m-D3 | A-40 | H | m-D3 | A-40 | H |
| 8877 | 0 | m-D3 | A-41 | H | m-D3 | A-41 | H |
| 8878 | 0 | m-D3 | A-42 | H | m-D3 | A-42 | H |
| 8879 | 0 | m-D3 | A-43 | H | m-D3 | A-43 | H |
| 8880 | 0 | m-D3 | A-44 | H | m-D3 | A-44 | H |
| 8881 | 0 | m-D3 | A-45 | H | m-D3 | A-45 | H |
| 8882 | 0 | m-D3 | A-46 | H | m-D3 | A-46 | H |
| 8883 | 0 | m-D3 | A-47 | H | m-D3 | A-47 | H |
| 8884 | 0 | m-D5 | A-1 | H | m-D5 | A-1 | H |
| 8885 | 0 | m-D5 | A-2 | H | m-D5 | A-2 | H |
| 8886 | 0 | m-D5 | A-3 | H | m-D5 | A-3 | H |
| 8887 | 0 | m-D5 | A-4 | H | m-D5 | A-4 | H |
| 8888 | 0 | m-D5 | A-5 | H | m-D5 | A-5 | H |
| 8889 | 0 | m-D5 | A-6 | H | m-D5 | A-6 | H |
| 8890 | 0 | m-D5 | A-7 | H | m-D5 | A-7 | H |
| 8891 | 0 | m-D5 | A-8 | H | m-D5 | A-8 | H |
| 8892 | 0 | m-D5 | A-9 | H | m-D5 | A-9 | H |
| 8893 | 0 | m-D5 | A-10 | H | m-D5 | A-10 | H |
| 8894 | 0 | m-D5 | A-11 | H | m-D5 | A-11 | H |
| 8895 | 0 | m-D5 | A-12 | H | m-D5 | A-12 | H |
| 8896 | 0 | m-D5 | A-13 | H | m-D5 | A-13 | H |
| 8897 | 0 | m-D5 | A-14 | H | m-D5 | A-14 | H |
| 8898 | 0 | m-D5 | A-15 | H | m-D5 | A-15 | H |
| 8899 | 0 | m-D5 | A-16 | H | m-D5 | A-16 | H |
| 8900 | 0 | m-D5 | A-17 | H | m-D5 | A-17 | H |
| 8901 | 0 | m-D5 | A-18 | H | m-D5 | A-18 | H |
| 8902 | 0 | m-D5 | A-19 | H | m-D5 | A-19 | H |
| 8903 | 0 | m-D5 | A-20 | H | m-D5 | A-20 | H |
| 8904 | 0 | m-D5 | A-21 | H | m-D5 | A-21 | H |
| 8905 | 0 | m-D5 | A-22 | H | m-D5 | A-22 | H |
| 8906 | 0 | m-D5 | A-23 | H | m-D5 | A-23 | H |
| 8907 | 0 | m-D5 | A-24 | H | m-D5 | A-24 | H |
| 8908 | 0 | m-D5 | A-25 | H | m-D5 | A-25 | H |
| 8909 | 0 | m-D5 | A-26 | H | m-D5 | A-26 | H |
| 8910 | 0 | m-D5 | A-27 | H | m-D5 | A-27 | H |
| 8911 | 0 | m-D5 | A-28 | H | m-D5 | A-28 | H |
| 8912 | 0 | m-D5 | A-29 | H | m-D5 | A-29 | H |
| 8913 | 0 | m-D5 | A-30 | H | m-D5 | A-30 | H |
| 8914 | 0 | m-D5 | A-31 | H | m-D5 | A-31 | H |
| 8915 | 0 | m-D5 | A-32 | H | m-D5 | A-32 | H |
| 8916 | 0 | m-D5 | A-33 | H | m-D5 | A-33 | H |
| 8917 | 0 | m-D5 | A-34 | H | m-D5 | A-34 | H |
| 8918 | 0 | m-D5 | A-35 | H | m-D5 | A-35 | H |
| 8919 | 0 | m-D5 | A-36 | H | m-D5 | A-36 | H |
| 8920 | 0 | m-D5 | A-37 | H | m-D5 | A-37 | H |
| 8921 | 0 | m-D5 | A-38 | H | m-D5 | A-38 | H |
| 8922 | 0 | m-D5 | A-39 | H | m-D5 | A-39 | H |
| 8923 | 0 | m-D5 | A-40 | H | m-D5 | A-40 | H |
| 8924 | 0 | m-D5 | A-41 | H | m-D5 | A-41 | H |
| 8925 | 0 | m-D5 | A-42 | H | m-D5 | A-42 | H |
| 8926 | 0 | m-D5 | A-43 | H | m-D5 | A-43 | H |
| 8927 | 0 | m-D5 | A-44 | H | m-D5 | A-44 | H |
| 8928 | 0 | m-D5 | A-45 | H | m-D5 | A-45 | H |
| 8929 | 0 | m-D5 | A-46 | H | m-D5 | A-46 | H |
| 8930 | 0 | m-D5 | A-47 | H | m-D5 | A-47 | H |
| 8931 | 0 | m-D9 | A-1 | H | m-D9 | A-1 | H |
| 8932 | 0 | m-D9 | A-2 | H | m-D9 | A-2 | H |
| 8933 | 0 | m-D9 | A-3 | H | m-D9 | A-3 | H |
| 8934 | 0 | m-D9 | A-4 | H | m-D9 | A-4 | H |
| 8935 | 0 | m-D9 | A-5 | H | m-D9 | A-5 | H |
| 8936 | 0 | m-D9 | A-6 | H | m-D9 | A-6 | H |
| 8937 | 0 | m-D9 | A-7 | H | m-D9 | A-7 | H |
| 8938 | 0 | m-D9 | A-8 | H | m-D9 | A-8 | H |
| 8939 | 0 | m-D9 | A-9 | H | m-D9 | A-9 | H |
| 8940 | 0 | m-D9 | A-10 | H | m-D9 | A-10 | H |
| 8941 | 0 | m-D9 | A-11 | H | m-D9 | A-11 | H |
| 8942 | 0 | m-D9 | A-12 | H | m-D9 | A-12 | H |
| 8943 | 0 | m-D9 | A-13 | H | m-D9 | A-13 | H |
| 8944 | 0 | m-D9 | A-14 | H | m-D9 | A-14 | H |
| 8945 | 0 | m-D9 | A-15 | H | m-D9 | A-15 | H |
| 8946 | 0 | m-D9 | A-16 | H | m-D9 | A-16 | H |
| 8947 | 0 | m-D9 | A-17 | H | m-D9 | A-17 | H |
| 8948 | 0 | m-D9 | A-18 | H | m-D9 | A-18 | H |
| 8949 | 0 | m-D9 | A-19 | H | m-D9 | A-19 | H |
| 8950 | 0 | m-D9 | A-20 | H | m-D9 | A-20 | H |
| 8951 | 0 | m-D9 | A-21 | H | m-D9 | A-21 | H |
| 8952 | 0 | m-D9 | A-22 | H | m-D9 | A-22 | H |
| 8953 | 0 | m-D9 | A-23 | H | m-D9 | A-23 | H |
| 8954 | 0 | m-D9 | A-24 | H | m-D9 | A-24 | H |
| 8955 | 0 | m-D9 | A-25 | H | m-D9 | A-25 | H |
| 8956 | 0 | m-D9 | A-26 | H | m-D9 | A-26 | H |
| 8957 | 0 | m-D9 | A-27 | H | m-D9 | A-27 | H |
| 8958 | 0 | m-D9 | A-28 | H | m-D9 | A-28 | H |
| 8959 | 0 | m-D9 | A-29 | H | m-D9 | A-29 | H |
| 8960 | 0 | m-D9 | A-30 | H | m-D9 | A-30 | H |
| 8961 | 0 | m-D9 | A-31 | H | m-D9 | A-31 | H |
| 8962 | 0 | m-D9 | A-32 | H | m-D9 | A-32 | H |
| 8963 | 0 | m-D9 | A-33 | H | m-D9 | A-33 | H |
| 8964 | 0 | m-D9 | A-34 | H | m-D9 | A-34 | H |
| 8965 | 0 | m-D9 | A-35 | H | m-D9 | A-35 | H |
| 8966 | 0 | m-D9 | A-36 | H | m-D9 | A-36 | H |
| 8967 | 0 | m-D9 | A-37 | H | m-D9 | A-37 | H |
| 8968 | 0 | m-D9 | A-38 | H | m-D9 | A-38 | H |
| 8969 | 0 | m-D9 | A-39 | H | m-D9 | A-39 | H |
| 8970 | 0 | m-D9 | A-40 | H | m-D9 | A-40 | H |
| 8971 | 0 | m-D9 | A-41 | H | m-D9 | A-41 | H |
| 8972 | 0 | m-D9 | A-42 | H | m-D9 | A-42 | H |
| 8973 | 0 | m-D9 | A-43 | H | m-D9 | A-43 | H |
| 8974 | 0 | m-D9 | A-44 | H | m-D9 | A-44 | H |
| 8975 | 0 | m-D9 | A-45 | H | m-D9 | A-45 | H |
| 8976 | 0 | m-D9 | A-46 | H | m-D9 | A-46 | H |
| 8977 | 0 | m-D9 | A-47 | H | m-D9 | A-47 | H |
| 8978 | 0 | m-D11 | A-1 | H | m-D11 | A-1 | H |
| 8979 | 0 | m-D11 | A-2 | H | m-D11 | A-2 | H |
| 8980 | 0 | m-D11 | A-3 | H | m-D11 | A-3 | H |
| 8981 | 0 | m-D11 | A-4 | H | m-D11 | A-4 | H |
| 8982 | 0 | m-D11 | A-5 | H | m-D11 | A-5 | H |
| 8983 | 0 | m-D11 | A-6 | H | m-D11 | A-6 | H |
| 8984 | 0 | m-D11 | A-7 | H | m-D11 | A-7 | H |
| 8985 | 0 | m-D11 | A-8 | H | m-D11 | A-8 | H |
| 8986 | 0 | m-D11 | A-9 | H | m-D11 | A-9 | H |
| 8987 | 0 | m-D11 | A-10 | H | m-D11 | A-10 | H |
| 8988 | 0 | m-D11 | A-11 | H | m-D11 | A-11 | H |
| 8989 | 0 | m-D11 | A-12 | H | m-D11 | A-12 | H |
| 8990 | 0 | m-D11 | A-13 | H | m-D11 | A-13 | H |
| 8991 | 0 | m-D11 | A-14 | H | m-D11 | A-14 | H |
| 8992 | 0 | m-D11 | A-15 | H | m-D11 | A-15 | H |
| 8993 | 0 | m-D11 | A-16 | H | m-D11 | A-16 | H |
| 8994 | 0 | m-D11 | A-17 | H | m-D11 | A-17 | H |
| 8995 | 0 | m-D11 | A-18 | H | m-D11 | A-18 | H |
| 8996 | 0 | m-D11 | A-19 | H | m-D11 | A-19 | H |
| 8997 | 0 | m-D11 | A-20 | H | m-D11 | A-20 | H |
| 8998 | 0 | m-D11 | A-21 | H | m-D11 | A-21 | H |
| 8999 | 0 | m-D11 | A-22 | H | m-D11 | A-22 | H |
| 9000 | 0 | m-D11 | A-23 | H | m-D11 | A-23 | H |
| 9001 | 0 | m-D11 | A-24 | H | m-D11 | A-24 | H |
| 9002 | 0 | m-D11 | A-25 | H | m-D11 | A-25 | H |
| 9003 | 0 | m-D11 | A-26 | H | m-D11 | A-26 | H |
| 9004 | 0 | m-D11 | A-27 | H | m-D11 | A-27 | H |
| 9005 | 0 | m-D11 | A-28 | H | m-D11 | A-28 | H |
| 9006 | 0 | m-D11 | A-29 | H | m-D11 | A-29 | H |
| 9007 | 0 | m-D11 | A-30 | H | m-D11 | A-30 | H |
| 9008 | 0 | m-D11 | A-31 | H | m-D11 | A-31 | H |
| 9009 | 0 | m-D11 | A-32 | H | m-D11 | A-32 | H |
| 9010 | 0 | m-D11 | A-33 | H | m-D11 | A-33 | H |
| 9011 | 0 | m-D11 | A-34 | H | m-D11 | A-34 | H |
| 9012 | 0 | m-D11 | A-35 | H | m-D11 | A-35 | H |
| 9013 | 0 | m-D11 | A-36 | H | m-D11 | A-36 | H |
| 9014 | 0 | m-D11 | A-37 | H | m-D11 | A-37 | H |
| 9015 | 0 | m-D11 | A-38 | H | m-D11 | A-38 | H |
| 9016 | 0 | m-D11 | A-39 | H | m-D11 | A-39 | H |
| 9017 | 0 | m-D11 | A-40 | H | m-D11 | A-40 | H |
| 9018 | 0 | m-D11 | A-41 | H | m-D11 | A-41 | H |
| 9019 | 0 | m-D11 | A-42 | H | m-D11 | A-42 | H |
| 9020 | 0 | m-D11 | A-43 | H | m-D11 | A-43 | H |
| 9021 | 0 | m-D11 | A-44 | H | m-D11 | A-44 | H |
| 9022 | 0 | m-D11 | A-45 | H | m-D11 | A-45 | H |
| 9023 | 0 | m-D11 | A-46 | H | m-D11 | A-46 | H |
| 9024 | 0 | m-D11 | A-47 | H | m-D11 | A-47 | H |
| 9025 | 0 | m-D12 | A-1 | H | m-D12 | A-1 | H |
| 9026 | 0 | m-D12 | A-2 | H | m-D12 | A-2 | H |
| 9027 | 0 | m-D12 | A-3 | H | m-D12 | A-3 | H |
| 9028 | 0 | m-D12 | A-4 | H | m-D12 | A-4 | H |
| 9029 | 0 | m-D12 | A-5 | H | m-D12 | A-5 | H |
| 9030 | 0 | m-D12 | A-6 | H | m-D12 | A-6 | H |
| 9031 | 0 | m-D12 | A-7 | H | m-D12 | A-7 | H |
| 9032 | 0 | m-D12 | A-8 | H | m-D12 | A-8 | H |
| 9033 | 0 | m-D12 | A-9 | H | m-D12 | A-9 | H |
| 9034 | 0 | m-D12 | A-10 | H | m-D12 | A-10 | H |
| 9035 | 0 | m-D12 | A-11 | H | m-D12 | A-11 | H |
| 9036 | 0 | m-D12 | A-12 | H | m-D12 | A-12 | H |
| 9037 | 0 | m-D12 | A-13 | H | m-D12 | A-13 | H |
| 9038 | 0 | m-D12 | A-14 | H | m-D12 | A-14 | H |
| 9039 | 0 | m-D12 | A-15 | H | m-D12 | A-15 | H |
| 9040 | 0 | m-D12 | A-16 | H | m-D12 | A-16 | H |
| 9041 | 0 | m-D12 | A-17 | H | m-D12 | A-17 | H |
| 9042 | 0 | m-D12 | A-18 | H | m-D12 | A-18 | H |
| 9043 | 0 | m-D12 | A-19 | H | m-D12 | A-19 | H |
| 9044 | 0 | m-D12 | A-20 | H | m-D12 | A-20 | H |
| 9045 | 0 | m-D12 | A-21 | H | m-D12 | A-21 | H |
| 9046 | 0 | m-D12 | A-22 | H | m-D12 | A-22 | H |
| 9047 | 0 | m-D12 | A-23 | H | m-D12 | A-23 | H |
| 9048 | 0 | m-D12 | A-24 | H | m-D12 | A-24 | H |
| 9049 | 0 | m-D12 | A-25 | H | m-D12 | A-25 | H |
| 9050 | 0 | m-D12 | A-26 | H | m-D12 | A-26 | H |
| 9051 | 0 | m-D12 | A-27 | H | m-D12 | A-27 | H |
| 9052 | 0 | m-D12 | A-28 | H | m-D12 | A-28 | H |
| 9053 | 0 | m-D12 | A-29 | H | m-D12 | A-29 | H |
| 9054 | 0 | m-D12 | A-30 | H | m-D12 | A-30 | H |
| 9055 | 0 | m-D12 | A-31 | H | m-D12 | A-31 | H |
| 9056 | 0 | m-D12 | A-32 | H | m-D12 | A-32 | H |
| 9057 | 0 | m-D12 | A-33 | H | m-D12 | A-33 | H |
| 9058 | 0 | m-D12 | A-34 | H | m-D12 | A-34 | H |
| 9059 | 0 | m-D12 | A-35 | H | m-D12 | A-35 | H |
| 9060 | 0 | m-D12 | A-36 | H | m-D12 | A-36 | H |
| 9061 | 0 | m-D12 | A-37 | H | m-D12 | A-37 | H |
| 9062 | 0 | m-D12 | A-38 | H | m-D12 | A-38 | H |
| 9063 | 0 | m-D12 | A-39 | H | m-D12 | A-39 | H |
| 9064 | 0 | m-D12 | A-40 | H | m-D12 | A-40 | H |
| 9065 | 0 | m-D12 | A-41 | H | m-D12 | A-41 | H |
| 9066 | 0 | m-D12 | A-42 | H | m-D12 | A-42 | H |
| 9067 | 0 | m-D12 | A-43 | H | m-D12 | A-43 | H |
| 9068 | 0 | m-D12 | A-44 | H | m-D12 | A-44 | H |
| 9069 | 0 | m-D12 | A-45 | H | m-D12 | A-45 | H |
| 9070 | 0 | m-D12 | A-46 | H | m-D12 | A-46 | H |
| 9071 | 0 | m-D12 | A-47 | H | m-D12 | A-47 | H |
| 9072 | 0 | m-D13 | A-1 | H | m-D13 | A-1 | H |
| 9073 | 0 | m-D13 | A-2 | H | m-D13 | A-2 | H |
| 9074 | 0 | m-D13 | A-3 | H | m-D13 | A-3 | H |
| 9075 | 0 | m-D13 | A-4 | H | m-D13 | A-4 | H |
| 9076 | 0 | m-D13 | A-5 | H | m-D13 | A-5 | H |
| 9077 | 0 | m-D13 | A-6 | H | m-D13 | A-6 | H |
| 9078 | 0 | m-D13 | A-7 | H | m-D13 | A-7 | H |
| 9079 | 0 | m-D13 | A-8 | H | m-D13 | A-8 | H |
| 9080 | 0 | m-D13 | A-9 | H | m-D13 | A-9 | H |
| 9081 | 0 | m-D13 | A-10 | H | m-D13 | A-10 | H |
| 9082 | 0 | m-D13 | A-11 | H | m-D13 | A-11 | H |
| 9083 | 0 | m-D13 | A-12 | H | m-D13 | A-12 | H |
| 9084 | 0 | m-D13 | A-13 | H | m-D13 | A-13 | H |
| 9085 | 0 | m-D13 | A-14 | H | m-D13 | A-14 | H |
| 9086 | 0 | m-D13 | A-15 | H | m-D13 | A-15 | H |
| 9087 | 0 | m-D13 | A-16 | H | m-D13 | A-16 | H |
| 9088 | 0 | m-D13 | A-17 | H | m-D13 | A-17 | H |
| 9089 | 0 | m-D13 | A-18 | H | m-D13 | A-18 | H |
| 9090 | 0 | m-D13 | A-19 | H | m-D13 | A-19 | H |
| 9091 | 0 | m-D13 | A-20 | H | m-D13 | A-20 | H |
| 9092 | 0 | m-D13 | A-21 | H | m-D13 | A-21 | H |
| 9093 | 0 | m-D13 | A-22 | H | m-D13 | A-22 | H |
| 9094 | 0 | m-D13 | A-23 | H | m-D13 | A-23 | H |
| 9095 | 0 | m-D13 | A-24 | H | m-D13 | A-24 | H |
| 9096 | 0 | m-D13 | A-25 | H | m-D13 | A-25 | H |
| 9097 | 0 | m-D13 | A-26 | H | m-D13 | A-26 | H |
| 9098 | 0 | m-D13 | A-27 | H | m-D13 | A-27 | H |
| 9099 | 0 | m-D13 | A-28 | H | m-D13 | A-28 | H |
| 9100 | 0 | m-D13 | A-29 | H | m-D13 | A-29 | H |
| 9101 | 0 | m-D13 | A-30 | H | m-D13 | A-30 | H |
| 9102 | 0 | m-D13 | A-31 | H | m-D13 | A-31 | H |
| 9103 | 0 | m-D13 | A-32 | H | m-D13 | A-32 | H |
| 9104 | 0 | m-D13 | A-33 | H | m-D13 | A-33 | H |
| 9105 | 0 | m-D13 | A-34 | H | m-D13 | A-34 | H |
| 9106 | 0 | m-D13 | A-35 | H | m-D13 | A-35 | H |
| 9107 | 0 | m-D13 | A-36 | H | m-D13 | A-36 | H |
| 9108 | 0 | m-D13 | A-37 | H | m-D13 | A-37 | H |
| 9109 | 0 | m-D13 | A-38 | H | m-D13 | A-38 | H |
| 9110 | 0 | m-D13 | A-39 | H | m-D13 | A-39 | H |
| 9111 | 0 | m-D13 | A-40 | H | m-D13 | A-40 | H |
| 9112 | 0 | m-D13 | A-41 | H | m-D13 | A-41 | H |
| 9113 | 0 | m-D13 | A-42 | H | m-D13 | A-42 | H |
| 9114 | 0 | m-D13 | A-43 | H | m-D13 | A-43 | H |
| 9115 | 0 | m-D13 | A-44 | H | m-D13 | A-44 | H |
| 9116 | 0 | m-D13 | A-45 | H | m-D13 | A-45 | H |
| 9117 | 0 | m-D13 | A-46 | H | m-D13 | A-46 | H |
| 9118 | 0 | m-D13 | A-47 | H | m-D13 | A-47 | H |
| 9119 | 0 | m-D14 | A-1 | H | m-D14 | A-1 | H |
| 9120 | 0 | m-D14 | A-2 | H | m-D14 | A-2 | H |
| 9121 | 0 | m-D14 | A-3 | H | m-D14 | A-3 | H |
| 9122 | 0 | m-D14 | A-4 | H | m-D14 | A-4 | H |
| 9123 | 0 | m-D14 | A-5 | H | m-D14 | A-5 | H |
| 9124 | 0 | m-D14 | A-6 | H | m-D14 | A-6 | H |
| 9125 | 0 | m-D14 | A-7 | H | m-D14 | A-7 | H |
| 9126 | 0 | m-D14 | A-8 | H | m-D14 | A-8 | H |
| 9127 | 0 | m-D14 | A-9 | H | m-D14 | A-9 | H |
| 9128 | 0 | m-D14 | A-10 | H | m-D14 | A-10 | H |
| 9129 | 0 | m-D14 | A-11 | H | m-D14 | A-11 | H |
| 9130 | 0 | m-D14 | A-12 | H | m-D14 | A-12 | H |
| 9131 | 0 | m-D14 | A-13 | H | m-D14 | A-13 | H |
| 9132 | 0 | m-D14 | A-14 | H | m-D14 | A-14 | H |
| 9133 | 0 | m-D14 | A-15 | H | m-D14 | A-15 | H |
| 9134 | 0 | m-D14 | A-16 | H | m-D14 | A-16 | H |
| 9135 | 0 | m-D14 | A-17 | H | m-D14 | A-17 | H |
| 9136 | 0 | m-D14 | A-18 | H | m-D14 | A-18 | H |
| 9137 | 0 | m-D14 | A-19 | H | m-D14 | A-19 | H |
| 9138 | 0 | m-D14 | A-20 | H | m-D14 | A-20 | H |
| 9139 | 0 | m-D14 | A-21 | H | m-D14 | A-21 | H |
| 9140 | 0 | m-D14 | A-22 | H | m-D14 | A-22 | H |
| 9141 | 0 | m-D14 | A-23 | H | m-D14 | A-23 | H |
| 9142 | 0 | m-D14 | A-24 | H | m-D14 | A-24 | H |
| 9143 | 0 | m-D14 | A-25 | H | m-D14 | A-25 | H |
| 9144 | 0 | m-D14 | A-26 | H | m-D14 | A-26 | H |
| 9145 | 0 | m-D14 | A-27 | H | m-D14 | A-27 | H |
| 9146 | 0 | m-D14 | A-28 | H | m-D14 | A-28 | H |
| 9147 | 0 | m-D14 | A-29 | H | m-D14 | A-29 | H |
| 9148 | 0 | m-D14 | A-30 | H | m-D14 | A-30 | H |
| 9149 | 0 | m-D14 | A-31 | H | m-D14 | A-31 | H |
| 9150 | 0 | m-D14 | A-32 | H | m-D14 | A-32 | H |
| 9151 | 0 | m-D14 | A-33 | H | m-D14 | A-33 | H |
| 9152 | 0 | m-D14 | A-34 | H | m-D14 | A-34 | H |
| 9153 | 0 | m-D14 | A-35 | H | m-D14 | A-35 | H |
| 9154 | 0 | m-D14 | A-36 | H | m-D14 | A-36 | H |
| 9155 | 0 | m-D14 | A-37 | H | m-D14 | A-37 | H |
| 9156 | 0 | m-D14 | A-38 | H | m-D14 | A-38 | H |
| 9157 | 0 | m-D14 | A-39 | H | m-D14 | A-39 | H |
| 9158 | 0 | m-D14 | A-40 | H | m-D14 | A-40 | H |
| 9159 | 0 | m-D14 | A-41 | H | m-D14 | A-41 | H |
| 9160 | 0 | m-D14 | A-42 | H | m-D14 | A-42 | H |
| 9161 | 0 | m-D14 | A-43 | H | m-D14 | A-43 | H |
| 9162 | 0 | m-D14 | A-44 | H | m-D14 | A-44 | H |
| 9163 | 0 | m-D14 | A-45 | H | m-D14 | A-45 | H |
| 9164 | 0 | m-D14 | A-46 | H | m-D14 | A-46 | H |
| 9165 | 0 | m-D14 | A-47 | H | m-D14 | A-47 | H |
| 9166 | 0 | m-D1 | m-D1 | H | A-1 | A-1 | H |
| 9167 | 0 | m-D1 | m-D1 | H | A-2 | A-2 | H |
| 9168 | 0 | m-D1 | m-D1 | H | A-3 | A-3 | H |
| 9169 | 0 | m-D1 | m-D1 | H | A-4 | A-4 | H |
| 9170 | 0 | m-D1 | m-D1 | H | A-5 | A-5 | H |
| 9171 | 0 | m-D1 | m-D1 | H | A-6 | A-6 | H |
| 9172 | 0 | m-D1 | m-D1 | H | A-7 | A-7 | H |
| 9173 | 0 | m-D1 | m-D1 | H | A-8 | A-8 | H |
| 9174 | 0 | m-D1 | m-D1 | H | A-9 | A-9 | H |
| 9175 | 0 | m-D1 | m-D1 | H | A-10 | A-10 | H |
| 9176 | 0 | m-D1 | m-D1 | H | A-11 | A-11 | H |
| 9177 | 0 | m-D1 | m-D1 | H | A-12 | A-12 | H |
| 9178 | 0 | m-D1 | m-D1 | H | A-13 | A-13 | H |
| 9179 | 0 | m-D1 | m-D1 | H | A-14 | A-14 | H |
| 9180 | 0 | m-D1 | m-D1 | H | A-15 | A-15 | H |
| 9181 | 0 | m-D1 | m-D1 | H | A-16 | A-16 | H |
| 9182 | 0 | m-D1 | m-D1 | H | A-17 | A-17 | H |
| 9183 | 0 | m-D1 | m-D1 | H | A-18 | A-18 | H |
| 9184 | 0 | m-D1 | m-D1 | H | A-19 | A-19 | H |
| 9185 | 0 | m-D1 | m-D1 | H | A-20 | A-20 | H |
| 9186 | 0 | m-D1 | m-D1 | H | A-21 | A-21 | H |
| 9187 | 0 | m-D1 | m-D1 | H | A-22 | A-22 | H |
| 9188 | 0 | m-D1 | m-D1 | H | A-23 | A-23 | H |
| 9189 | 0 | m-D1 | m-D1 | H | A-24 | A-24 | H |
| 9190 | 0 | m-D1 | m-D1 | H | A-25 | A-25 | H |
| 9191 | 0 | m-D1 | m-D1 | H | A-26 | A-26 | H |
| 9192 | 0 | m-D1 | m-D1 | H | A-27 | A-27 | H |
| 9193 | 0 | m-D1 | m-D1 | H | A-28 | A-28 | H |
| 9194 | 0 | m-D1 | m-D1 | H | A-29 | A-29 | H |
| 9195 | 0 | m-D1 | m-D1 | H | A-30 | A-30 | H |
| 9196 | 0 | m-D1 | m-D1 | H | A-31 | A-31 | H |
| 9197 | 0 | m-D1 | m-D1 | H | A-32 | A-32 | H |
| 9198 | 0 | m-D1 | m-D1 | H | A-33 | A-33 | H |
| 9199 | 0 | m-D1 | m-D1 | H | A-34 | A-34 | H |
| 9200 | 0 | m-D1 | m-D1 | H | A-35 | A-35 | H |
| 9201 | 0 | m-D1 | m-D1 | H | A-36 | A-36 | H |
| 9202 | 0 | m-D1 | m-D1 | H | A-37 | A-37 | H |
| 9203 | 0 | m-D1 | m-D1 | H | A-38 | A-38 | H |
| 9204 | 0 | m-D1 | m-D1 | H | A-39 | A-39 | H |
| 9205 | 0 | m-D1 | m-D1 | H | A-40 | A-40 | H |
| 9206 | 0 | m-D1 | m-D1 | H | A-41 | A-41 | H |
| 9207 | 0 | m-D1 | m-D1 | H | A-42 | A-42 | H |
| 9208 | 0 | m-D1 | m-D1 | H | A-43 | A-43 | H |
| 9209 | 0 | m-D1 | m-D1 | H | A-44 | A-44 | H |
| 9210 | 0 | m-D1 | m-D1 | H | A-45 | A-45 | H |
| 9211 | 0 | m-D1 | m-D1 | H | A-46 | A-46 | H |
| 9212 | 0 | m-D1 | m-D1 | H | A-47 | A-47 | H |
| 9213 | 0 | m-D2 | m-D2 | H | A-1 | A-1 | H |
| 9214 | 0 | m-D2 | m-D2 | H | A-2 | A-2 | H |
| 9215 | 0 | m-D2 | m-D2 | H | A-3 | A-3 | H |
| 9216 | 0 | m-D2 | m-D2 | H | A-4 | A-4 | H |
| 9217 | 0 | m-D2 | m-D2 | H | A-5 | A-5 | H |
| 9218 | 0 | m-D2 | m-D2 | H | A-6 | A-6 | H |
| 9219 | 0 | m-D2 | m-D2 | H | A-7 | A-7 | H |
| 9220 | 0 | m-D2 | m-D2 | H | A-8 | A-8 | H |
| 9221 | 0 | m-D2 | m-D2 | H | A-9 | A-9 | H |
| 9222 | 0 | m-D2 | m-D2 | H | A-10 | A-10 | H |
| 9223 | 0 | m-D2 | m-D2 | H | A-11 | A-11 | H |
| 9224 | 0 | m-D2 | m-D2 | H | A-12 | A-12 | H |
| 9225 | 0 | m-D2 | m-D2 | H | A-13 | A-13 | H |
| 9226 | 0 | m-D2 | m-D2 | H | A-14 | A-14 | H |
| 9227 | 0 | m-D2 | m-D2 | H | A-15 | A-15 | H |
| 9228 | 0 | m-D2 | m-D2 | H | A-16 | A-16 | H |
| 9229 | 0 | m-D2 | m-D2 | H | A-17 | A-17 | H |
| 9230 | 0 | m-D2 | m-D2 | H | A-18 | A-18 | H |
| 9231 | 0 | m-D2 | m-D2 | H | A-19 | A-19 | H |
| 9232 | 0 | m-D2 | m-D2 | H | A-20 | A-20 | H |
| 9233 | 0 | m-D2 | m-D2 | H | A-21 | A-21 | H |
| 9234 | 0 | m-D2 | m-D2 | H | A-22 | A-22 | H |
| 9235 | 0 | m-D2 | m-D2 | H | A-23 | A-23 | H |
| 9236 | 0 | m-D2 | m-D2 | H | A-24 | A-24 | H |
| 9237 | 0 | m-D2 | m-D2 | H | A-25 | A-25 | H |
| 9238 | 0 | m-D2 | m-D2 | H | A-26 | A-26 | H |
| 9239 | 0 | m-D2 | m-D2 | H | A-27 | A-27 | H |
| 9240 | 0 | m-D2 | m-D2 | H | A-28 | A-28 | H |
| 9241 | 0 | m-D2 | m-D2 | H | A-29 | A-29 | H |
| 9242 | 0 | m-D2 | m-D2 | H | A-30 | A-30 | H |
| 9243 | 0 | m-D2 | m-D2 | H | A-31 | A-31 | H |
| 9244 | 0 | m-D2 | m-D2 | H | A-32 | A-32 | H |
| 9245 | 0 | m-D2 | m-D2 | H | A-33 | A-33 | H |
| 9246 | 0 | m-D2 | m-D2 | H | A-34 | A-34 | H |
| 9247 | 0 | m-D2 | m-D2 | H | A-35 | A-35 | H |
| 9248 | 0 | m-D2 | m-D2 | H | A-36 | A-36 | H |
| 9249 | 0 | m-D2 | m-D2 | H | A-37 | A-37 | H |
| 9250 | 0 | m-D2 | m-D2 | H | A-38 | A-38 | H |
| 9251 | 0 | m-D2 | m-D2 | H | A-39 | A-39 | H |
| 9252 | 0 | m-D2 | m-D2 | H | A-40 | A-40 | H |
| 9253 | 0 | m-D2 | m-D2 | H | A-41 | A-41 | H |
| 9254 | 0 | m-D2 | m-D2 | H | A-42 | A-42 | H |
| 9255 | 0 | m-D2 | m-D2 | H | A-43 | A-43 | H |
| 9256 | 0 | m-D2 | m-D2 | H | A-44 | A-44 | H |
| 9257 | 0 | m-D2 | m-D2 | H | A-45 | A-45 | H |
| 9258 | 0 | m-D2 | m-D2 | H | A-46 | A-46 | H |
| 9259 | 0 | m-D2 | m-D2 | H | A-47 | A-47 | H |
| 9260 | 0 | m-D3 | m-D3 | H | A-1 | A-1 | H |
| 9261 | 0 | m-D3 | m-D3 | H | A-2 | A-2 | H |
| 9262 | 0 | m-D3 | m-D3 | H | A-3 | A-3 | H |
| 9263 | 0 | m-D3 | m-D3 | H | A-4 | A-4 | H |
| 9264 | 0 | m-D3 | m-D3 | H | A-5 | A-5 | H |
| 9265 | 0 | m-D3 | m-D3 | H | A-6 | A-6 | H |
| 9266 | 0 | m-D3 | m-D3 | H | A-7 | A-7 | H |
| 9267 | 0 | m-D3 | m-D3 | H | A-8 | A-8 | H |
| 9268 | 0 | m-D3 | m-D3 | H | A-9 | A-9 | H |
| 9269 | 0 | m-D3 | m-D3 | H | A-10 | A-10 | H |
| 9270 | 0 | m-D3 | m-D3 | H | A-11 | A-11 | H |
| 9271 | 0 | m-D3 | m-D3 | H | A-12 | A-12 | H |
| 9272 | 0 | m-D3 | m-D3 | H | A-13 | A-13 | H |
| 9273 | 0 | m-D3 | m-D3 | H | A-14 | A-14 | H |
| 9274 | 0 | m-D3 | m-D3 | H | A-15 | A-15 | H |
| 9275 | 0 | m-D3 | m-D3 | H | A-16 | A-16 | H |
| 9276 | 0 | m-D3 | m-D3 | H | A-17 | A-17 | H |
| 9277 | 0 | m-D3 | m-D3 | H | A-18 | A-18 | H |
| 9278 | 0 | m-D3 | m-D3 | H | A-19 | A-19 | H |
| 9279 | 0 | m-D3 | m-D3 | H | A-20 | A-20 | H |
| 9280 | 0 | m-D3 | m-D3 | H | A-21 | A-21 | H |
| 9281 | 0 | m-D3 | m-D3 | H | A-22 | A-22 | H |
| 9282 | 0 | m-D3 | m-D3 | H | A-23 | A-23 | H |
| 9283 | 0 | m-D3 | m-D3 | H | A-24 | A-24 | H |
| 9284 | 0 | m-D3 | m-D3 | H | A-25 | A-25 | H |
| 9285 | 0 | m-D3 | m-D3 | H | A-26 | A-26 | H |
| 9286 | 0 | m-D3 | m-D3 | H | A-27 | A-27 | H |
| 9287 | 0 | m-D3 | m-D3 | H | A-28 | A-28 | H |
| 9288 | 0 | m-D3 | m-D3 | H | A-29 | A-29 | H |
| 9289 | 0 | m-D3 | m-D3 | H | A-30 | A-30 | H |
| 9290 | 0 | m-D3 | m-D3 | H | A-31 | A-31 | H |
| 9291 | 0 | m-D3 | m-D3 | H | A-32 | A-32 | H |
| 9292 | 0 | m-D3 | m-D3 | H | A-33 | A-33 | H |
| 9293 | 0 | m-D3 | m-D3 | H | A-34 | A-34 | H |
| 9294 | 0 | m-D3 | m-D3 | H | A-35 | A-35 | H |
| 9295 | 0 | m-D3 | m-D3 | H | A-36 | A-36 | H |
| 9296 | 0 | m-D3 | m-D3 | H | A-37 | A-37 | H |
| 9297 | 0 | m-D3 | m-D3 | H | A-38 | A-38 | H |
| 9298 | 0 | m-D3 | m-D3 | H | A-39 | A-39 | H |
| 9299 | 0 | m-D3 | m-D3 | H | A-40 | A-40 | H |
| 9300 | 0 | m-D3 | m-D3 | H | A-41 | A-41 | H |
| 9301 | 0 | m-D3 | m-D3 | H | A-42 | A-42 | H |
| 9302 | 0 | m-D3 | m-D3 | H | A-43 | A-43 | H |
| 9303 | 0 | m-D3 | m-D3 | H | A-44 | A-44 | H |
| 9304 | 0 | m-D3 | m-D3 | H | A-45 | A-45 | H |
| 9305 | 0 | m-D3 | m-D3 | H | A-46 | A-46 | H |
| 9306 | 0 | m-D3 | m-D3 | H | A-47 | A-47 | H |
| 9307 | 0 | m-D5 | m-D5 | H | A-1 | A-1 | H |
| 9308 | 0 | m-D5 | m-D5 | H | A-2 | A-2 | H |
| 9309 | 0 | m-D5 | m-D5 | H | A-3 | A-3 | H |
| 9310 | 0 | m-D5 | m-D5 | H | A-4 | A-4 | H |
| 9311 | 0 | m-D5 | m-D5 | H | A-5 | A-5 | H |
| 9312 | 0 | m-D5 | m-D5 | H | A-6 | A-6 | H |
| 9313 | 0 | m-D5 | m-D5 | H | A-7 | A-7 | H |
| 9314 | 0 | m-D5 | m-D5 | H | A-8 | A-8 | H |
| 9315 | 0 | m-D5 | m-D5 | H | A-9 | A-9 | H |
| 9316 | 0 | m-D5 | m-D5 | H | A-10 | A-10 | H |
| 9317 | 0 | m-D5 | m-D5 | H | A-11 | A-11 | H |
| 9318 | 0 | m-D5 | m-D5 | H | A-12 | A-12 | H |
| 9319 | 0 | m-D5 | m-D5 | H | A-13 | A-13 | H |
| 9320 | 0 | m-D5 | m-D5 | H | A-14 | A-14 | H |
| 9321 | 0 | m-D5 | m-D5 | H | A-15 | A-15 | H |
| 9322 | 0 | m-D5 | m-D5 | H | A-16 | A-16 | H |
| 9323 | 0 | m-D5 | m-D5 | H | A-17 | A-17 | H |
| 9324 | 0 | m-D5 | m-D5 | H | A-18 | A-18 | H |
| 9325 | 0 | m-D5 | m-D5 | H | A-19 | A-19 | H |
| 9326 | 0 | m-D5 | m-D5 | H | A-20 | A-20 | H |
| 9327 | 0 | m-D5 | m-D5 | H | A-21 | A-21 | H |
| 9328 | 0 | m-D5 | m-D5 | H | A-22 | A-22 | H |
| 9329 | 0 | m-D5 | m-D5 | H | A-23 | A-23 | H |
| 9330 | 0 | m-D5 | m-D5 | H | A-24 | A-24 | H |
| 9331 | 0 | m-D5 | m-D5 | H | A-25 | A-25 | H |
| 9332 | 0 | m-D5 | m-D5 | H | A-26 | A-26 | H |
| 9333 | 0 | m-D5 | m-D5 | H | A-27 | A-27 | H |
| 9334 | 0 | m-D5 | m-D5 | H | A-28 | A-28 | H |
| 9335 | 0 | m-D5 | m-D5 | H | A-29 | A-29 | H |
| 9336 | 0 | m-D5 | m-D5 | H | A-30 | A-30 | H |
| 9337 | 0 | m-D5 | m-D5 | H | A-31 | A-31 | H |
| 9338 | 0 | m-D5 | m-D5 | H | A-32 | A-32 | H |
| 9339 | 0 | m-D5 | m-D5 | H | A-33 | A-33 | H |
| 9340 | 0 | m-D5 | m-D5 | H | A-34 | A-34 | H |
| 9341 | 0 | m-D5 | m-D5 | H | A-35 | A-35 | H |
| 9342 | 0 | m-D5 | m-D5 | H | A-36 | A-36 | H |
| 9343 | 0 | m-D5 | m-D5 | H | A-37 | A-37 | H |
| 9344 | 0 | m-D5 | m-D5 | H | A-38 | A-38 | H |
| 9345 | 0 | m-D5 | m-D5 | H | A-39 | A-39 | H |
| 9346 | 0 | m-D5 | m-D5 | H | A-40 | A-40 | H |
| 9347 | 0 | m-D5 | m-D5 | H | A-41 | A-41 | H |
| 9348 | 0 | m-D5 | m-D5 | H | A-42 | A-42 | H |
| 9349 | 0 | m-D5 | m-D5 | H | A-43 | A-43 | H |
| 9350 | 0 | m-D5 | m-D5 | H | A-44 | A-44 | H |
| 9351 | 0 | m-D5 | m-D5 | H | A-45 | A-45 | H |
| 9352 | 0 | m-D5 | m-D5 | H | A-46 | A-46 | H |
| 9353 | 0 | m-D5 | m-D5 | H | A-47 | A-47 | H |
| 9354 | 0 | m-D9 | m-D9 | H | A-1 | A-1 | H |
| 9355 | 0 | m-D9 | m-D9 | H | A-2 | A-2 | H |
| 9356 | 0 | m-D9 | m-D9 | H | A-3 | A-3 | H |
| 9357 | 0 | m-D9 | m-D9 | H | A-4 | A-4 | H |
| 9358 | 0 | m-D9 | m-D9 | H | A-5 | A-5 | H |
| 9359 | 0 | m-D9 | m-D9 | H | A-6 | A-6 | H |
| 9360 | 0 | m-D9 | m-D9 | H | A-7 | A-7 | H |
| 9361 | 0 | m-D9 | m-D9 | H | A-8 | A-8 | H |
| 9362 | 0 | m-D9 | m-D9 | H | A-9 | A-9 | H |
| 9363 | 0 | m-D9 | m-D9 | H | A-10 | A-10 | H |
| 9364 | 0 | m-D9 | m-D9 | H | A-11 | A-11 | H |
| 9365 | 0 | m-D9 | m-D9 | H | A-12 | A-12 | H |
| 9366 | 0 | m-D9 | m-D9 | H | A-13 | A-13 | H |
| 9367 | 0 | m-D9 | m-D9 | H | A-14 | A-14 | H |
| 9368 | 0 | m-D9 | m-D9 | H | A-15 | A-15 | H |
| 9369 | 0 | m-D9 | m-D9 | H | A-16 | A-16 | H |
| 9370 | 0 | m-D9 | m-D9 | H | A-17 | A-17 | H |
| 9371 | 0 | m-D9 | m-D9 | H | A-18 | A-18 | H |
| 9372 | 0 | m-D9 | m-D9 | H | A-19 | A-19 | H |
| 9373 | 0 | m-D9 | m-D9 | H | A-20 | A-20 | H |
| 9374 | 0 | m-D9 | m-D9 | H | A-21 | A-21 | H |
| 9375 | 0 | m-D9 | m-D9 | H | A-22 | A-22 | H |
| 9376 | 0 | m-D9 | m-D9 | H | A-23 | A-23 | H |
| 9377 | 0 | m-D9 | m-D9 | H | A-24 | A-24 | H |
| 9378 | 0 | m-D9 | m-D9 | H | A-25 | A-25 | H |
| 9379 | 0 | m-D9 | m-D9 | H | A-26 | A-26 | H |
| 9380 | 0 | m-D9 | m-D9 | H | A-27 | A-27 | H |
| 9381 | 0 | m-D9 | m-D9 | H | A-28 | A-28 | H |
| 9382 | 0 | m-D9 | m-D9 | H | A-29 | A-29 | H |
| 9383 | 0 | m-D9 | m-D9 | H | A-30 | A-30 | H |
| 9384 | 0 | m-D9 | m-D9 | H | A-31 | A-31 | H |
| 9385 | 0 | m-D9 | m-D9 | H | A-32 | A-32 | H |
| 9386 | 0 | m-D9 | m-D9 | H | A-33 | A-33 | H |
| 9387 | 0 | m-D9 | m-D9 | H | A-34 | A-34 | H |
| 9388 | 0 | m-D9 | m-D9 | H | A-35 | A-35 | H |
| 9389 | 0 | m-D9 | m-D9 | H | A-36 | A-36 | H |
| 9390 | 0 | m-D9 | m-D9 | H | A-37 | A-37 | H |
| 9391 | 0 | m-D9 | m-D9 | H | A-38 | A-38 | H |
| 9392 | 0 | m-D9 | m-D9 | H | A-39 | A-39 | H |
| 9393 | 0 | m-D9 | m-D9 | H | A-40 | A-40 | H |
| 9394 | 0 | m-D9 | m-D9 | H | A-41 | A-41 | H |
| 9395 | 0 | m-D9 | m-D9 | H | A-42 | A-42 | H |
| 9396 | 0 | m-D9 | m-D9 | H | A-43 | A-43 | H |
| 9397 | 0 | m-D9 | m-D9 | H | A-44 | A-44 | H |
| 9398 | 0 | m-D9 | m-D9 | H | A-45 | A-45 | H |
| 9399 | 0 | m-D9 | m-D9 | H | A-46 | A-46 | H |
| 9400 | 0 | m-D9 | m-D9 | H | A-47 | A-47 | H |
| 9401 | 0 | m-D11 | m-D11 | H | A-1 | A-1 | H |
| 9402 | 0 | m-D11 | m-D11 | H | A-2 | A-2 | H |
| 9403 | 0 | m-D11 | m-D11 | H | A-3 | A-3 | H |
| 9404 | 0 | m-D11 | m-D11 | H | A-4 | A-4 | H |
| 9405 | 0 | m-D11 | m-D11 | H | A-5 | A-5 | H |
| 9406 | 0 | m-D11 | m-D11 | H | A-6 | A-6 | H |
| 9407 | 0 | m-D11 | m-D11 | H | A-7 | A-7 | H |
| 9408 | 0 | m-D11 | m-D11 | H | A-8 | A-8 | H |
| 9409 | 0 | m-D11 | m-D11 | H | A-9 | A-9 | H |
| 9410 | 0 | m-D11 | m-D11 | H | A-10 | A-10 | H |
| 9411 | 0 | m-D11 | m-D11 | H | A-11 | A-11 | H |
| 9412 | 0 | m-D11 | m-D11 | H | A-12 | A-12 | H |
| 9413 | 0 | m-D11 | m-D11 | H | A-13 | A-13 | H |
| 9414 | 0 | m-D11 | m-D11 | H | A-14 | A-14 | H |
| 9415 | 0 | m-D11 | m-D11 | H | A-15 | A-15 | H |
| 9416 | 0 | m-D11 | m-D11 | H | A-16 | A-16 | H |
| 9417 | 0 | m-D11 | m-D11 | H | A-17 | A-17 | H |
| 9418 | 0 | m-D11 | m-D11 | H | A-18 | A-18 | H |
| 9419 | 0 | m-D11 | m-D11 | H | A-19 | A-19 | H |
| 9420 | 0 | m-D11 | m-D11 | H | A-20 | A-20 | H |
| 9421 | 0 | m-D11 | m-D11 | H | A-21 | A-21 | H |
| 9422 | 0 | m-D11 | m-D11 | H | A-22 | A-22 | H |
| 9423 | 0 | m-D11 | m-D11 | H | A-23 | A-23 | H |
| 9424 | 0 | m-D11 | m-D11 | H | A-24 | A-24 | H |
| 9425 | 0 | m-D11 | m-D11 | H | A-25 | A-25 | H |
| 9426 | 0 | m-D11 | m-D11 | H | A-26 | A-26 | H |
| 9427 | 0 | m-D11 | m-D11 | H | A-27 | A-27 | H |
| 9428 | 0 | m-D11 | m-D11 | H | A-28 | A-28 | H |
| 9429 | 0 | m-D11 | m-D11 | H | A-29 | A-29 | H |
| 9430 | 0 | m-D11 | m-D11 | H | A-30 | A-30 | H |
| 9431 | 0 | m-D11 | m-D11 | H | A-31 | A-31 | H |
| 9432 | 0 | m-D11 | m-D11 | H | A-32 | A-32 | H |
| 9433 | 0 | m-D11 | m-D11 | H | A-33 | A-33 | H |
| 9434 | 0 | m-D11 | m-D11 | H | A-34 | A-34 | H |
| 9435 | 0 | m-D11 | m-D11 | H | A-35 | A-35 | H |
| 9436 | 0 | m-D11 | m-D11 | H | A-36 | A-36 | H |
| 9437 | 0 | m-D11 | m-D11 | H | A-37 | A-37 | H |
| 9438 | 0 | m-D11 | m-D11 | H | A-38 | A-38 | H |
| 9439 | 0 | m-D11 | m-D11 | H | A-39 | A-39 | H |
| 9440 | 0 | m-D11 | m-D11 | H | A-40 | A-40 | H |
| 9441 | 0 | m-D11 | m-D11 | H | A-41 | A-41 | H |
| 9442 | 0 | m-D11 | m-D11 | H | A-42 | A-42 | H |
| 9443 | 0 | m-D11 | m-D11 | H | A-43 | A-43 | H |
| 9444 | 0 | m-D11 | m-D11 | H | A-44 | A-44 | H |
| 9445 | 0 | m-D11 | m-D11 | H | A-45 | A-45 | H |
| 9446 | 0 | m-D11 | m-D11 | H | A-46 | A-46 | H |
| 9447 | 0 | m-D11 | m-D11 | H | A-47 | A-47 | H |
| 9448 | 0 | m-D12 | m-D12 | H | A-1 | A-1 | H |
| 9449 | 0 | m-D12 | m-D12 | H | A-2 | A-2 | H |
| 9450 | 0 | m-D12 | m-D12 | H | A-3 | A-3 | H |
| 9451 | 0 | m-D12 | m-D12 | H | A-4 | A-4 | H |
| 9452 | 0 | m-D12 | m-D12 | H | A-5 | A-5 | H |
| 9453 | 0 | m-D12 | m-D12 | H | A-6 | A-6 | H |
| 9454 | 0 | m-D12 | m-D12 | H | A-7 | A-7 | H |
| 9455 | 0 | m-D12 | m-D12 | H | A-8 | A-8 | H |
| 9456 | 0 | m-D12 | m-D12 | H | A-9 | A-9 | H |
| 9457 | 0 | m-D12 | m-D12 | H | A-10 | A-10 | H |
| 9458 | 0 | m-D12 | m-D12 | H | A-11 | A-11 | H |
| 9459 | 0 | m-D12 | m-D12 | H | A-12 | A-12 | H |
| 9460 | 0 | m-D12 | m-D12 | H | A-13 | A-13 | H |
| 9461 | 0 | m-D12 | m-D12 | H | A-14 | A-14 | H |
| 9462 | 0 | m-D12 | m-D12 | H | A-15 | A-15 | H |
| 9463 | 0 | m-D12 | m-D12 | H | A-16 | A-16 | H |
| 9464 | 0 | m-D12 | m-D12 | H | A-17 | A-17 | H |
| 9465 | 0 | m-D12 | m-D12 | H | A-18 | A-18 | H |
| 9466 | 0 | m-D12 | m-D12 | H | A-19 | A-19 | H |
| 9467 | 0 | m-D12 | m-D12 | H | A-20 | A-20 | H |
| 9468 | 0 | m-D12 | m-D12 | H | A-21 | A-21 | H |
| 9469 | 0 | m-D12 | m-D12 | H | A-22 | A-22 | H |
| 9470 | 0 | m-D12 | m-D12 | H | A-23 | A-23 | H |
| 9471 | 0 | m-D12 | m-D12 | H | A-24 | A-24 | H |
| 9472 | 0 | m-D12 | m-D12 | H | A-25 | A-25 | H |
| 9473 | 0 | m-D12 | m-D12 | H | A-26 | A-26 | H |
| 9474 | 0 | m-D12 | m-D12 | H | A-27 | A-27 | H |
| 9475 | 0 | m-D12 | m-D12 | H | A-28 | A-28 | H |
| 9476 | 0 | m-D12 | m-D12 | H | A-29 | A-29 | H |
| 9477 | 0 | m-D12 | m-D12 | H | A-30 | A-30 | H |
| 9478 | 0 | m-D12 | m-D12 | H | A-31 | A-31 | H |
| 9479 | 0 | m-D12 | m-D12 | H | A-32 | A-32 | H |
| 9480 | 0 | m-D12 | m-D12 | H | A-33 | A-33 | H |
| 9481 | 0 | m-D12 | m-D12 | H | A-34 | A-34 | H |
| 9482 | 0 | m-D12 | m-D12 | H | A-35 | A-35 | H |
| 9483 | 0 | m-D12 | m-D12 | H | A-36 | A-36 | H |
| 9484 | 0 | m-D12 | m-D12 | H | A-37 | A-37 | H |
| 9485 | 0 | m-D12 | m-D12 | H | A-38 | A-38 | H |
| 9486 | 0 | m-D12 | m-D12 | H | A-39 | A-39 | H |
| 9487 | 0 | m-D12 | m-D12 | H | A-40 | A-40 | H |
| 9488 | 0 | m-D12 | m-D12 | H | A-41 | A-41 | H |
| 9489 | 0 | m-D12 | m-D12 | H | A-42 | A-42 | H |
| 9490 | 0 | m-D12 | m-D12 | H | A-43 | A-43 | H |
| 9491 | 0 | m-D12 | m-D12 | H | A-44 | A-44 | H |
| 9492 | 0 | m-D12 | m-D12 | H | A-45 | A-45 | H |
| 9493 | 0 | m-D12 | m-D12 | H | A-46 | A-46 | H |
| 9494 | 0 | m-D12 | m-D12 | H | A-47 | A-47 | H |
| 9495 | 0 | m-D13 | m-D13 | H | A-1 | A-1 | H |
| 9496 | 0 | m-D13 | m-D13 | H | A-2 | A-2 | H |
| 9497 | 0 | m-D13 | m-D13 | H | A-3 | A-3 | H |
| 9498 | 0 | m-D13 | m-D13 | H | A-4 | A-4 | H |
| 9499 | 0 | m-D13 | m-D13 | H | A-5 | A-5 | H |
| 9500 | 0 | m-D13 | m-D13 | H | A-6 | A-6 | H |
| 9501 | 0 | m-D13 | m-D13 | H | A-7 | A-7 | H |
| 9502 | 0 | m-D13 | m-D13 | H | A-8 | A-8 | H |
| 9503 | 0 | m-D13 | m-D13 | H | A-9 | A-9 | H |
| 9504 | 0 | m-D13 | m-D13 | H | A-10 | A-10 | H |
| 9505 | 0 | m-D13 | m-D13 | H | A-11 | A-11 | H |
| 9506 | 0 | m-D13 | m-D13 | H | A-12 | A-12 | H |
| 9507 | 0 | m-D13 | m-D13 | H | A-13 | A-13 | H |
| 9508 | 0 | m-D13 | m-D13 | H | A-14 | A-14 | H |
| 9509 | 0 | m-D13 | m-D13 | H | A-15 | A-15 | H |
| 9510 | 0 | m-D13 | m-D13 | H | A-16 | A-16 | H |
| 9511 | 0 | m-D13 | m-D13 | H | A-17 | A-17 | H |
| 9512 | 0 | m-D13 | m-D13 | H | A-18 | A-18 | H |
| 9513 | 0 | m-D13 | m-D13 | H | A-19 | A-19 | H |
| 9514 | 0 | m-D13 | m-D13 | H | A-20 | A-20 | H |
| 9515 | 0 | m-D13 | m-D13 | H | A-21 | A-21 | H |
| 9516 | 0 | m-D13 | m-D13 | H | A-22 | A-22 | H |
| 9517 | 0 | m-D13 | m-D13 | H | A-23 | A-23 | H |
| 9518 | 0 | m-D13 | m-D13 | H | A-24 | A-24 | H |
| 9519 | 0 | m-D13 | m-D13 | H | A-25 | A-25 | H |
| 9520 | 0 | m-D13 | m-D13 | H | A-26 | A-26 | H |
| 9521 | 0 | m-D13 | m-D13 | H | A-27 | A-27 | H |
| 9522 | 0 | m-D13 | m-D13 | H | A-28 | A-28 | H |
| 9523 | 0 | m-D13 | m-D13 | H | A-29 | A-29 | H |
| 9524 | 0 | m-D13 | m-D13 | H | A-30 | A-30 | H |
| 9525 | 0 | m-D13 | m-D13 | H | A-31 | A-31 | H |
| 9526 | 0 | m-D13 | m-D13 | H | A-32 | A-32 | H |
| 9527 | 0 | m-D13 | m-D13 | H | A-33 | A-33 | H |
| 9528 | 0 | m-D13 | m-D13 | H | A-34 | A-34 | H |
| 9529 | 0 | m-D13 | m-D13 | H | A-35 | A-35 | H |
| 9530 | 0 | m-D13 | m-D13 | H | A-36 | A-36 | H |
| 9531 | 0 | m-D13 | m-D13 | H | A-37 | A-37 | H |
| 9532 | 0 | m-D13 | m-D13 | H | A-38 | A-38 | H |
| 9533 | 0 | m-D13 | m-D13 | H | A-39 | A-39 | H |
| 9534 | 0 | m-D13 | m-D13 | H | A-40 | A-40 | H |
| 9535 | 0 | m-D13 | m-D13 | H | A-41 | A-41 | H |
| 9536 | 0 | m-D13 | m-D13 | H | A-42 | A-42 | H |
| 9537 | 0 | m-D13 | m-D13 | H | A-43 | A-43 | H |
| 9538 | 0 | m-D13 | m-D13 | H | A-44 | A-44 | H |
| 9539 | 0 | m-D13 | m-D13 | H | A-45 | A-45 | H |
| 9540 | 0 | m-D13 | m-D13 | H | A-46 | A-46 | H |
| 9541 | 0 | m-D13 | m-D13 | H | A-47 | A-47 | H |
| 9542 | 0 | m-D14 | m-D14 | H | A-1 | A-1 | H |
| 9543 | 0 | m-D14 | m-D14 | H | A-2 | A-2 | H |
| 9544 | 0 | m-D14 | m-D14 | H | A-3 | A-3 | H |
| 9545 | 0 | m-D14 | m-D14 | H | A-4 | A-4 | H |
| 9546 | 0 | m-D14 | m-D14 | H | A-5 | A-5 | H |
| 9547 | 0 | m-D14 | m-D14 | H | A-6 | A-6 | H |
| 9548 | 0 | m-D14 | m-D14 | H | A-7 | A-7 | H |
| 9549 | 0 | m-D14 | m-D14 | H | A-8 | A-8 | H |
| 9550 | 0 | m-D14 | m-D14 | H | A-9 | A-9 | H |
| 9551 | 0 | m-D14 | m-D14 | H | A-10 | A-10 | H |
| 9552 | 0 | m-D14 | m-D14 | H | A-11 | A-11 | H |
| 9553 | 0 | m-D14 | m-D14 | H | A-12 | A-12 | H |
| 9554 | 0 | m-D14 | m-D14 | H | A-13 | A-13 | H |
| 9555 | 0 | m-D14 | m-D14 | H | A-14 | A-14 | H |
| 9556 | 0 | m-D14 | m-D14 | H | A-15 | A-15 | H |
| 9557 | 0 | m-D14 | m-D14 | H | A-16 | A-16 | H |
| 9558 | 0 | m-D14 | m-D14 | H | A-17 | A-17 | H |
| 9559 | 0 | m-D14 | m-D14 | H | A-18 | A-18 | H |
| 9560 | 0 | m-D14 | m-D14 | H | A-19 | A-19 | H |
| 9561 | 0 | m-D14 | m-D14 | H | A-20 | A-20 | H |
| 9562 | 0 | m-D14 | m-D14 | H | A-21 | A-21 | H |
| 9563 | 0 | m-D14 | m-D14 | H | A-22 | A-22 | H |
| 9564 | 0 | m-D14 | m-D14 | H | A-23 | A-23 | H |
| 9565 | 0 | m-D14 | m-D14 | H | A-24 | A-24 | H |
| 9566 | 0 | m-D14 | m-D14 | H | A-25 | A-25 | H |
| 9567 | 0 | m-D14 | m-D14 | H | A-26 | A-26 | H |
| 9568 | 0 | m-D14 | m-D14 | H | A-27 | A-27 | H |
| 9569 | 0 | m-D14 | m-D14 | H | A-28 | A-28 | H |
| 9570 | 0 | m-D14 | m-D14 | H | A-29 | A-29 | H |
| 9571 | 0 | m-D14 | m-D14 | H | A-30 | A-30 | H |
| 9572 | 0 | m-D14 | m-D14 | H | A-31 | A-31 | H |
| 9573 | 0 | m-D14 | m-D14 | H | A-32 | A-32 | H |
| 9574 | 0 | m-D14 | m-D14 | H | A-33 | A-33 | H |
| 9575 | 0 | m-D14 | m-D14 | H | A-34 | A-34 | H |
| 9576 | 0 | m-D14 | m-D14 | H | A-35 | A-35 | H |
| 9577 | 0 | m-D14 | m-D14 | H | A-36 | A-36 | H |
| 9578 | 0 | m-D14 | m-D14 | H | A-37 | A-37 | H |
| 9579 | 0 | m-D14 | m-D14 | H | A-38 | A-38 | H |
| 9580 | 0 | m-D14 | m-D14 | H | A-39 | A-39 | H |
| 9581 | 0 | m-D14 | m-D14 | H | A-40 | A-40 | H |
| 9582 | 0 | m-D14 | m-D14 | H | A-41 | A-41 | H |
| 9583 | 0 | m-D14 | m-D14 | H | A-42 | A-42 | H |
| 9584 | 0 | m-D14 | m-D14 | H | A-43 | A-43 | H |
| 9585 | 0 | m-D14 | m-D14 | H | A-44 | A-44 | H |
| 9586 | 0 | m-D14 | m-D14 | H | A-45 | A-45 | H |
| 9587 | 0 | m-D14 | m-D14 | H | A-46 | A-46 | H |
| 9588 | 0 | m-D14 | m-D14 | H | A-47 | A-47 | H |
| 9589 | 0 | m-D1 | A-1 | H | A-1 | m-D1 | H |
| 9590 | 0 | m-D1 | A-2 | H | A-2 | m-D1 | H |
| 9591 | 0 | m-D1 | A-3 | H | A-3 | m-D1 | H |
| 9592 | 0 | m-D1 | A-4 | H | A-4 | m-D1 | H |
| 9593 | 0 | m-D1 | A-5 | H | A-5 | m-D1 | H |
| 9594 | 0 | m-D1 | A-6 | H | A-6 | m-D1 | H |
| 9595 | 0 | m-D1 | A-7 | H | A-7 | m-D1 | H |
| 9596 | 0 | m-D1 | A-8 | H | A-8 | m-D1 | H |
| 9597 | 0 | m-D1 | A-9 | H | A-9 | m-D1 | H |
| 9598 | 0 | m-D1 | A-10 | H | A-10 | m-D1 | H |
| 9599 | 0 | m-D1 | A-11 | H | A-11 | m-D1 | H |
| 9600 | 0 | m-D1 | A-12 | H | A-12 | m-D1 | H |
| 9601 | 0 | m-D1 | A-13 | H | A-13 | m-D1 | H |
| 9602 | 0 | m-D1 | A-14 | H | A-14 | m-D1 | H |
| 9603 | 0 | m-D1 | A-15 | H | A-15 | m-D1 | H |
| 9604 | 0 | m-D1 | A-16 | H | A-16 | m-D1 | H |
| 9605 | 0 | m-D1 | A-17 | H | A-17 | m-D1 | H |
| 9606 | 0 | m-D1 | A-18 | H | A-18 | m-D1 | H |
| 9607 | 0 | m-D1 | A-19 | H | A-19 | m-D1 | H |
| 9608 | 0 | m-D1 | A-20 | H | A-20 | m-D1 | H |
| 9609 | 0 | m-D1 | A-21 | H | A-21 | m-D1 | H |
| 9610 | 0 | m-D1 | A-22 | H | A-22 | m-D1 | H |
| 9611 | 0 | m-D1 | A-23 | H | A-23 | m-D1 | H |
| 9612 | 0 | m-D1 | A-24 | H | A-24 | m-D1 | H |
| 9613 | 0 | m-D1 | A-25 | H | A-25 | m-D1 | H |
| 9614 | 0 | m-D1 | A-26 | H | A-26 | m-D1 | H |
| 9615 | 0 | m-D1 | A-27 | H | A-27 | m-D1 | H |
| 9616 | 0 | m-D1 | A-28 | H | A-28 | m-D1 | H |
| 9617 | 0 | m-D1 | A-29 | H | A-29 | m-D1 | H |
| 9618 | 0 | m-D1 | A-30 | H | A-30 | m-D1 | H |
| 9619 | 0 | m-D1 | A-31 | H | A-31 | m-D1 | H |
| 9620 | 0 | m-D1 | A-32 | H | A-32 | m-D1 | H |
| 9621 | 0 | m-D1 | A-33 | H | A-33 | m-D1 | H |
| 9622 | 0 | m-D1 | A-34 | H | A-34 | m-D1 | H |
| 9623 | 0 | m-D1 | A-35 | H | A-35 | m-D1 | H |
| 9624 | 0 | m-D1 | A-36 | H | A-36 | m-D1 | H |
| 9625 | 0 | m-D1 | A-37 | H | A-37 | m-D1 | H |
| 9626 | 0 | m-D1 | A-38 | H | A-38 | m-D1 | H |
| 9627 | 0 | m-D1 | A-39 | H | A-39 | m-D1 | H |
| 9628 | 0 | m-D1 | A-40 | H | A-40 | m-D1 | H |
| 9629 | 0 | m-D1 | A-41 | H | A-41 | m-D1 | H |
| 9630 | 0 | m-D1 | A-42 | H | A-42 | m-D1 | H |
| 9631 | 0 | m-D1 | A-43 | H | A-43 | m-D1 | H |
| 9632 | 0 | m-D1 | A-44 | H | A-44 | m-D1 | H |
| 9633 | 0 | m-D1 | A-45 | H | A-45 | m-D1 | H |
| 9634 | 0 | m-D1 | A-46 | H | A-46 | m-D1 | H |
| 9635 | 0 | m-D1 | A-47 | H | A-47 | m-D1 | H |
| 9636 | 0 | m-D2 | A-1 | H | A-1 | m-D2 | H |
| 9637 | 0 | m-D2 | A-2 | H | A-2 | m-D2 | H |
| 9638 | 0 | m-D2 | A-3 | H | A-3 | m-D2 | H |
| 9639 | 0 | m-D2 | A-4 | H | A-4 | m-D2 | H |
| 9640 | 0 | m-D2 | A-5 | H | A-5 | m-D2 | H |
| 9641 | 0 | m-D2 | A-6 | H | A-6 | m-D2 | H |
| 9642 | 0 | m-D2 | A-7 | H | A-7 | m-D2 | H |
| 9643 | 0 | m-D2 | A-8 | H | A-8 | m-D2 | H |
| 9644 | 0 | m-D2 | A-9 | H | A-9 | m-D2 | H |
| 9645 | 0 | m-D2 | A-10 | H | A-10 | m-D2 | H |
| 9646 | 0 | m-D2 | A-11 | H | A-11 | m-D2 | H |
| 9647 | 0 | m-D2 | A-12 | H | A-12 | m-D2 | H |
| 9648 | 0 | m-D2 | A-13 | H | A-13 | m-D2 | H |
| 9649 | 0 | m-D2 | A-14 | H | A-14 | m-D2 | H |
| 9650 | 0 | m-D2 | A-15 | H | A-15 | m-D2 | H |
| 9651 | 0 | m-D2 | A-16 | H | A-16 | m-D2 | H |
| 9652 | 0 | m-D2 | A-17 | H | A-17 | m-D2 | H |
| 9653 | 0 | m-D2 | A-18 | H | A-18 | m-D2 | H |
| 9654 | 0 | m-D2 | A-19 | H | A-19 | m-D2 | H |
| 9655 | 0 | m-D2 | A-20 | H | A-20 | m-D2 | H |
| 9656 | 0 | m-D2 | A-21 | H | A-21 | m-D2 | H |
| 9657 | 0 | m-D2 | A-22 | H | A-22 | m-D2 | H |
| 9658 | 0 | m-D2 | A-23 | H | A-23 | m-D2 | H |
| 9659 | 0 | m-D2 | A-24 | H | A-24 | m-D2 | H |
| 9660 | 0 | m-D2 | A-25 | H | A-25 | m-D2 | H |
| 9661 | 0 | m-D2 | A-26 | H | A-26 | m-D2 | H |
| 9662 | 0 | m-D2 | A-27 | H | A-27 | m-D2 | H |
| 9663 | 0 | m-D2 | A-28 | H | A-28 | m-D2 | H |
| 9664 | 0 | m-D2 | A-29 | H | A-29 | m-D2 | H |
| 9665 | 0 | m-D2 | A-30 | H | A-30 | m-D2 | H |
| 9666 | 0 | m-D2 | A-31 | H | A-31 | m-D2 | H |
| 9667 | 0 | m-D2 | A-32 | H | A-32 | m-D2 | H |
| 9668 | 0 | m-D2 | A-33 | H | A-33 | m-D2 | H |
| 9669 | 0 | m-D2 | A-34 | H | A-34 | m-D2 | H |
| 9670 | 0 | m-D2 | A-35 | H | A-35 | m-D2 | H |
| 9671 | 0 | m-D2 | A-36 | H | A-36 | m-D2 | H |
| 9672 | 0 | m-D2 | A-37 | H | A-37 | m-D2 | H |
| 9673 | 0 | m-D2 | A-38 | H | A-38 | m-D2 | H |
| 9674 | 0 | m-D2 | A-39 | H | A-39 | m-D2 | H |
| 9675 | 0 | m-D2 | A-40 | H | A-40 | m-D2 | H |
| 9676 | 0 | m-D2 | A-41 | H | A-41 | m-D2 | H |
| 9677 | 0 | m-D2 | A-42 | H | A-42 | m-D2 | H |
| 9678 | 0 | m-D2 | A-43 | H | A-43 | m-D2 | H |
| 9679 | 0 | m-D2 | A-44 | H | A-44 | m-D2 | H |
| 9680 | 0 | m-D2 | A-45 | H | A-45 | m-D2 | H |
| 9681 | 0 | m-D2 | A-46 | H | A-46 | m-D2 | H |
| 9682 | 0 | m-D2 | A-47 | H | A-47 | m-D2 | H |
| 9683 | 0 | m-D3 | A-1 | H | A-1 | m-D3 | H |
| 9684 | 0 | m-D3 | A-2 | H | A-2 | m-D3 | H |
| 9685 | 0 | m-D3 | A-3 | H | A-3 | m-D3 | H |
| 9686 | 0 | m-D3 | A-4 | H | A-4 | m-D3 | H |
| 9687 | 0 | m-D3 | A-5 | H | A-5 | m-D3 | H |
| 9688 | 0 | m-D3 | A-6 | H | A-6 | m-D3 | H |
| 9689 | 0 | m-D3 | A-7 | H | A-7 | m-D3 | H |
| 9690 | 0 | m-D3 | A-8 | H | A-8 | m-D3 | H |
| 9691 | 0 | m-D3 | A-9 | H | A-9 | m-D3 | H |
| 9692 | 0 | m-D3 | A-10 | H | A-10 | m-D3 | H |
| 9693 | 0 | m-D3 | A-11 | H | A-11 | m-D3 | H |
| 9694 | 0 | m-D3 | A-12 | H | A-12 | m-D3 | H |
| 9695 | 0 | m-D3 | A-13 | H | A-13 | m-D3 | H |
| 9696 | 0 | m-D3 | A-14 | H | A-14 | m-D3 | H |
| 9697 | 0 | m-D3 | A-15 | H | A-15 | m-D3 | H |
| 9698 | 0 | m-D3 | A-16 | H | A-16 | m-D3 | H |
| 9699 | 0 | m-D3 | A-17 | H | A-17 | m-D3 | H |
| 9700 | 0 | m-D3 | A-18 | H | A-18 | m-D3 | H |
| 9701 | 0 | m-D3 | A-19 | H | A-19 | m-D3 | H |
| 9702 | 0 | m-D3 | A-20 | H | A-20 | m-D3 | H |
| 9703 | 0 | m-D3 | A-21 | H | A-21 | m-D3 | H |
| 9704 | 0 | m-D3 | A-22 | H | A-22 | m-D3 | H |
| 9705 | 0 | m-D3 | A-23 | H | A-23 | m-D3 | H |
| 9706 | 0 | m-D3 | A-24 | H | A-24 | m-D3 | H |
| 9707 | 0 | m-D3 | A-25 | H | A-25 | m-D3 | H |
| 9708 | 0 | m-D3 | A-26 | H | A-26 | m-D3 | H |
| 9709 | 0 | m-D3 | A-27 | H | A-27 | m-D3 | H |
| 9710 | 0 | m-D3 | A-28 | H | A-28 | m-D3 | H |
| 9711 | 0 | m-D3 | A-29 | H | A-29 | m-D3 | H |
| 9712 | 0 | m-D3 | A-30 | H | A-30 | m-D3 | H |
| 9713 | 0 | m-D3 | A-31 | H | A-31 | m-D3 | H |
| 9714 | 0 | m-D3 | A-32 | H | A-32 | m-D3 | H |
| 9715 | 0 | m-D3 | A-33 | H | A-33 | m-D3 | H |
| 9716 | 0 | m-D3 | A-34 | H | A-34 | m-D3 | H |
| 9717 | 0 | m-D3 | A-35 | H | A-35 | m-D3 | H |
| 9718 | 0 | m-D3 | A-36 | H | A-36 | m-D3 | H |
| 9719 | 0 | m-D3 | A-37 | H | A-37 | m-D3 | H |
| 9720 | 0 | m-D3 | A-38 | H | A-38 | m-D3 | H |
| 9721 | 0 | m-D3 | A-39 | H | A-39 | m-D3 | H |
| 9722 | 0 | m-D3 | A-40 | H | A-40 | m-D3 | H |
| 9723 | 0 | m-D3 | A-41 | H | A-41 | m-D3 | H |
| 9724 | 0 | m-D3 | A-42 | H | A-42 | m-D3 | H |
| 9725 | 0 | m-D3 | A-43 | H | A-43 | m-D3 | H |
| 9726 | 0 | m-D3 | A-44 | H | A-44 | m-D3 | H |
| 9727 | 0 | m-D3 | A-45 | H | A-45 | m-D3 | H |
| 9728 | 0 | m-D3 | A-46 | H | A-46 | m-D3 | H |
| 9729 | 0 | m-D3 | A-47 | H | A-47 | m-D3 | H |
| 9730 | 0 | m-D5 | A-1 | H | A-1 | m-D5 | H |
| 9731 | 0 | m-D5 | A-2 | H | A-2 | m-D5 | H |
| 9732 | 0 | m-D5 | A-3 | H | A-3 | m-D5 | H |
| 9733 | 0 | m-D5 | A-4 | H | A-4 | m-D5 | H |
| 9734 | 0 | m-D5 | A-5 | H | A-5 | m-D5 | H |
| 9735 | 0 | m-D5 | A-6 | H | A-6 | m-D5 | H |
| 9736 | 0 | m-D5 | A-7 | H | A-7 | m-D5 | H |
| 9737 | 0 | m-D5 | A-8 | H | A-8 | m-D5 | H |
| 9738 | 0 | m-D5 | A-9 | H | A-9 | m-D5 | H |
| 9739 | 0 | m-D5 | A-10 | H | A-10 | m-D5 | H |
| 9740 | 0 | m-D5 | A-11 | H | A-11 | m-D5 | H |
| 9741 | 0 | m-D5 | A-12 | H | A-12 | m-D5 | H |
| 9742 | 0 | m-D5 | A-13 | H | A-13 | m-D5 | H |
| 9743 | 0 | m-D5 | A-14 | H | A-14 | m-D5 | H |
| 9744 | 0 | m-D5 | A-15 | H | A-15 | m-D5 | H |
| 9745 | 0 | m-D5 | A-16 | H | A-16 | m-D5 | H |
| 9746 | 0 | m-D5 | A-17 | H | A-17 | m-D5 | H |
| 9747 | 0 | m-D5 | A-18 | H | A-18 | m-D5 | H |
| 9748 | 0 | m-D5 | A-19 | H | A-19 | m-D5 | H |
| 9749 | 0 | m-D5 | A-20 | H | A-20 | m-D5 | H |
| 9750 | 0 | m-D5 | A-21 | H | A-21 | m-D5 | H |
| 9751 | 0 | m-D5 | A-22 | H | A-22 | m-D5 | H |
| 9752 | 0 | m-D5 | A-23 | H | A-23 | m-D5 | H |
| 9753 | 0 | m-D5 | A-24 | H | A-24 | m-D5 | H |
| 9754 | 0 | m-D5 | A-25 | H | A-25 | m-D5 | H |
| 9755 | 0 | m-D5 | A-26 | H | A-26 | m-D5 | H |
| 9756 | 0 | m-D5 | A-27 | H | A-27 | m-D5 | H |
| 9757 | 0 | m-D5 | A-28 | H | A-28 | m-D5 | H |
| 9758 | 0 | m-D5 | A-29 | H | A-29 | m-D5 | H |
| 9759 | 0 | m-D5 | A-30 | H | A-30 | m-D5 | H |
| 9760 | 0 | m-D5 | A-31 | H | A-31 | m-D5 | H |
| 9761 | 0 | m-D5 | A-32 | H | A-32 | m-D5 | H |
| 9762 | 0 | m-D5 | A-33 | H | A-33 | m-D5 | H |
| 9763 | 0 | m-D5 | A-34 | H | A-34 | m-D5 | H |
| 9764 | 0 | m-D5 | A-35 | H | A-35 | m-D5 | H |
| 9765 | 0 | m-D5 | A-36 | H | A-36 | m-D5 | H |
| 9766 | 0 | m-D5 | A-37 | H | A-37 | m-D5 | H |
| 9767 | 0 | m-D5 | A-38 | H | A-38 | m-D5 | H |
| 9768 | 0 | m-D5 | A-39 | H | A-39 | m-D5 | H |
| 9769 | 0 | m-D5 | A-40 | H | A-40 | m-D5 | H |
| 9770 | 0 | m-D5 | A-41 | H | A-41 | m-D5 | H |
| 9771 | 0 | m-D5 | A-42 | H | A-42 | m-D5 | H |
| 9772 | 0 | m-D5 | A-43 | H | A-43 | m-D5 | H |
| 9773 | 0 | m-D5 | A-44 | H | A-44 | m-D5 | H |
| 9774 | 0 | m-D5 | A-45 | H | A-45 | m-D5 | H |
| 9775 | 0 | m-D5 | A-46 | H | A-46 | m-D5 | H |
| 9776 | 0 | m-D5 | A-47 | H | A-47 | m-D5 | H |
| 9777 | 0 | m-D9 | A-1 | H | A-1 | m-D9 | H |
| 9778 | 0 | m-D9 | A-2 | H | A-2 | m-D9 | H |
| 9779 | 0 | m-D9 | A-3 | H | A-3 | m-D9 | H |
| 9780 | 0 | m-D9 | A-4 | H | A-4 | m-D9 | H |
| 9781 | 0 | m-D9 | A-5 | H | A-5 | m-D9 | H |
| 9782 | 0 | m-D9 | A-6 | H | A-6 | m-D9 | H |
| 9783 | 0 | m-D9 | A-7 | H | A-7 | m-D9 | H |
| 9784 | 0 | m-D9 | A-8 | H | A-8 | m-D9 | H |
| 9785 | 0 | m-D9 | A-9 | H | A-9 | m-D9 | H |
| 9786 | 0 | m-D9 | A-10 | H | A-10 | m-D9 | H |
| 9787 | 0 | m-D9 | A-11 | H | A-11 | m-D9 | H |
| 9788 | 0 | m-D9 | A-12 | H | A-12 | m-D9 | H |
| 9789 | 0 | m-D9 | A-13 | H | A-13 | m-D9 | H |
| 9790 | 0 | m-D9 | A-14 | H | A-14 | m-D9 | H |
| 9791 | 0 | m-D9 | A-15 | H | A-15 | m-D9 | H |
| 9792 | 0 | m-D9 | A-16 | H | A-16 | m-D9 | H |
| 9793 | 0 | m-D9 | A-17 | H | A-17 | m-D9 | H |
| 9794 | 0 | m-D9 | A-18 | H | A-18 | m-D9 | H |
| 9795 | 0 | m-D9 | A-19 | H | A-19 | m-D9 | H |
| 9796 | 0 | m-D9 | A-20 | H | A-20 | m-D9 | H |
| 9797 | 0 | m-D9 | A-21 | H | A-21 | m-D9 | H |
| 9798 | 0 | m-D9 | A-22 | H | A-22 | m-D9 | H |
| 9799 | 0 | m-D9 | A-23 | H | A-23 | m-D9 | H |
| 9800 | 0 | m-D9 | A-24 | H | A-24 | m-D9 | H |
| 9801 | 0 | m-D9 | A-25 | H | A-25 | m-D9 | H |
| 9802 | 0 | m-D9 | A-26 | H | A-26 | m-D9 | H |
| 9803 | 0 | m-D9 | A-27 | H | A-27 | m-D9 | H |
| 9804 | 0 | m-D9 | A-28 | H | A-28 | m-D9 | H |
| 9805 | 0 | m-D9 | A-29 | H | A-29 | m-D9 | H |
| 9806 | 0 | m-D9 | A-30 | H | A-30 | m-D9 | H |
| 9807 | 0 | m-D9 | A-31 | H | A-31 | m-D9 | H |
| 9808 | 0 | m-D9 | A-32 | H | A-32 | m-D9 | H |
| 9809 | 0 | m-D9 | A-33 | H | A-33 | m-D9 | H |
| 9810 | 0 | m-D9 | A-34 | H | A-34 | m-D9 | H |
| 9811 | 0 | m-D9 | A-35 | H | A-35 | m-D9 | H |
| 9812 | 0 | m-D9 | A-36 | H | A-36 | m-D9 | H |
| 9813 | 0 | m-D9 | A-37 | H | A-37 | m-D9 | H |
| 9814 | 0 | m-D9 | A-38 | H | A-38 | m-D9 | H |
| 9815 | 0 | m-D9 | A-39 | H | A-39 | m-D9 | H |
| 9816 | 0 | m-D9 | A-40 | H | A-40 | m-D9 | H |
| 9817 | 0 | m-D9 | A-41 | H | A-41 | m-D9 | H |
| 9818 | 0 | m-D9 | A-42 | H | A-42 | m-D9 | H |
| 9819 | 0 | m-D9 | A-43 | H | A-43 | m-D9 | H |
| 9820 | 0 | m-D9 | A-44 | H | A-44 | m-D9 | H |
| 9821 | 0 | m-D9 | A-45 | H | A-45 | m-D9 | H |
| 9822 | 0 | m-D9 | A-46 | H | A-46 | m-D9 | H |
| 9823 | 0 | m-D9 | A-47 | H | A-47 | m-D9 | H |
| 9824 | 0 | m-D11 | A-1 | H | A-1 | m-D11 | H |
| 9825 | 0 | m-D11 | A-2 | H | A-2 | m-D11 | H |
| 9826 | 0 | m-D11 | A-3 | H | A-3 | m-D11 | H |
| 9827 | 0 | m-D11 | A-4 | H | A-4 | m-D11 | H |
| 9828 | 0 | m-D11 | A-5 | H | A-5 | m-D11 | H |
| 9829 | 0 | m-D11 | A-6 | H | A-6 | m-D11 | H |
| 9830 | 0 | m-D11 | A-7 | H | A-7 | m-D11 | H |
| 9831 | 0 | m-D11 | A-8 | H | A-8 | m-D11 | H |
| 9832 | 0 | m-D11 | A-9 | H | A-9 | m-D11 | H |
| 9833 | 0 | m-D11 | A-10 | H | A-10 | m-D11 | H |
| 9834 | 0 | m-D11 | A-11 | H | A-11 | m-D11 | H |
| 9835 | 0 | m-D11 | A-12 | H | A-12 | m-D11 | H |
| 9836 | 0 | m-D11 | A-13 | H | A-13 | m-D11 | H |
| 9837 | 0 | m-D11 | A-14 | H | A-14 | m-D11 | H |
| 9838 | 0 | m-D11 | A-15 | H | A-15 | m-D11 | H |
| 9839 | 0 | m-D11 | A-16 | H | A-16 | m-D11 | H |
| 9840 | 0 | m-D11 | A-17 | H | A-17 | m-D11 | H |
| 9841 | 0 | m-D11 | A-18 | H | A-18 | m-D11 | H |
| 9842 | 0 | m-D11 | A-19 | H | A-19 | m-D11 | H |
| 9843 | 0 | m-D11 | A-20 | H | A-20 | m-D11 | H |
| 9844 | 0 | m-D11 | A-21 | H | A-21 | m-D11 | H |
| 9845 | 0 | m-D11 | A-22 | H | A-22 | m-D11 | H |
| 9846 | 0 | m-D11 | A-23 | H | A-23 | m-D11 | H |
| 9847 | 0 | m-D11 | A-24 | H | A-24 | m-D11 | H |
| 9848 | 0 | m-D11 | A-25 | H | A-25 | m-D11 | H |
| 9849 | 0 | m-D11 | A-26 | H | A-26 | m-D11 | H |
| 9850 | 0 | m-D11 | A-27 | H | A-27 | m-D11 | H |
| 9851 | 0 | m-D11 | A-28 | H | A-28 | m-D11 | H |
| 9852 | 0 | m-D11 | A-29 | H | A-29 | m-D11 | H |
| 9853 | 0 | m-D11 | A-30 | H | A-30 | m-D11 | H |
| 9854 | 0 | m-D11 | A-31 | H | A-31 | m-D11 | H |
| 9855 | 0 | m-D11 | A-32 | H | A-32 | m-D11 | H |
| 9856 | 0 | m-D11 | A-33 | H | A-33 | m-D11 | H |
| 9857 | 0 | m-D11 | A-34 | H | A-34 | m-D11 | H |
| 9858 | 0 | m-D11 | A-35 | H | A-35 | m-D11 | H |
| 9859 | 0 | m-D11 | A-36 | H | A-36 | m-D11 | H |
| 9860 | 0 | m-D11 | A-37 | H | A-37 | m-D11 | H |
| 9861 | 0 | m-D11 | A-38 | H | A-38 | m-D11 | H |
| 9862 | 0 | m-D11 | A-39 | H | A-39 | m-D11 | H |
| 9863 | 0 | m-D11 | A-40 | H | A-40 | m-D11 | H |
| 9864 | 0 | m-D11 | A-41 | H | A-41 | m-D11 | H |
| 9865 | 0 | m-D11 | A-42 | H | A-42 | m-D11 | H |
| 9866 | 0 | m-D11 | A-43 | H | A-43 | m-D11 | H |
| 9867 | 0 | m-D11 | A-44 | H | A-44 | m-D11 | H |
| 9868 | 0 | m-D11 | A-45 | H | A-45 | m-D11 | H |
| 9869 | 0 | m-D11 | A-46 | H | A-46 | m-D11 | H |
| 9870 | 0 | m-D11 | A-47 | H | A-47 | m-D11 | H |
| 9871 | 0 | m-D12 | A-1 | H | A-1 | m-D12 | H |
| 9872 | 0 | m-D12 | A-2 | H | A-2 | m-D12 | H |
| 9873 | 0 | m-D12 | A-3 | H | A-3 | m-D12 | H |
| 9874 | 0 | m-D12 | A-4 | H | A-4 | m-D12 | H |
| 9875 | 0 | m-D12 | A-5 | H | A-5 | m-D12 | H |
| 9876 | 0 | m-D12 | A-6 | H | A-6 | m-D12 | H |
| 9877 | 0 | m-D12 | A-7 | H | A-7 | m-D12 | H |
| 9878 | 0 | m-D12 | A-8 | H | A-8 | m-D12 | H |
| 9879 | 0 | m-D12 | A-9 | H | A-9 | m-D12 | H |
| 9880 | 0 | m-D12 | A-10 | H | A-10 | m-D12 | H |
| 9881 | 0 | m-D12 | A-11 | H | A-11 | m-D12 | H |
| 9882 | 0 | m-D12 | A-12 | H | A-12 | m-D12 | H |
| 9883 | 0 | m-D12 | A-13 | H | A-13 | m-D12 | H |
| 9884 | 0 | m-D12 | A-14 | H | A-14 | m-D12 | H |
| 9885 | 0 | m-D12 | A-15 | H | A-15 | m-D12 | H |
| 9886 | 0 | m-D12 | A-16 | H | A-16 | m-D12 | H |
| 9887 | 0 | m-D12 | A-17 | H | A-17 | m-D12 | H |
| 9888 | 0 | m-D12 | A-18 | H | A-18 | m-D12 | H |
| 9889 | 0 | m-D12 | A-19 | H | A-19 | m-D12 | H |
| 9890 | 0 | m-D12 | A-20 | H | A-20 | m-D12 | H |
| 9891 | 0 | m-D12 | A-21 | H | A-21 | m-D12 | H |
| 9892 | 0 | m-D12 | A-22 | H | A-22 | m-D12 | H |
| 9893 | 0 | m-D12 | A-23 | H | A-23 | m-D12 | H |
| 9894 | 0 | m-D12 | A-24 | H | A-24 | m-D12 | H |
| 9895 | 0 | m-D12 | A-25 | H | A-25 | m-D12 | H |
| 9896 | 0 | m-D12 | A-26 | H | A-26 | m-D12 | H |
| 9897 | 0 | m-D12 | A-27 | H | A-27 | m-D12 | H |
| 9898 | 0 | m-D12 | A-28 | H | A-28 | m-D12 | H |
| 9899 | 0 | m-D12 | A-29 | H | A-29 | m-D12 | H |
| 9900 | 0 | m-D12 | A-30 | H | A-30 | m-D12 | H |
| 9901 | 0 | m-D12 | A-31 | H | A-31 | m-D12 | H |
| 9902 | 0 | m-D12 | A-32 | H | A-32 | m-D12 | H |
| 9903 | 0 | m-D12 | A-33 | H | A-33 | m-D12 | H |
| 9904 | 0 | m-D12 | A-34 | H | A-34 | m-D12 | H |
| 9905 | 0 | m-D12 | A-35 | H | A-35 | m-D12 | H |
| 9906 | 0 | m-D12 | A-36 | H | A-36 | m-D12 | H |
| 9907 | 0 | m-D12 | A-37 | H | A-37 | m-D12 | H |
| 9908 | 0 | m-D12 | A-38 | H | A-38 | m-D12 | H |
| 9909 | 0 | m-D12 | A-39 | H | A-39 | m-D12 | H |
| 9910 | 0 | m-D12 | A-40 | H | A-40 | m-D12 | H |
| 9911 | 0 | m-D12 | A-41 | H | A-41 | m-D12 | H |
| 9912 | 0 | m-D12 | A-42 | H | A-42 | m-D12 | H |
| 9913 | 0 | m-D12 | A-43 | H | A-43 | m-D12 | H |
| 9914 | 0 | m-D12 | A-44 | H | A-44 | m-D12 | H |
| 9915 | 0 | m-D12 | A-45 | H | A-45 | m-D12 | H |
| 9916 | 0 | m-D12 | A-46 | H | A-46 | m-D12 | H |
| 9917 | 0 | m-D12 | A-47 | H | A-47 | m-D12 | H |
| 9918 | 0 | m-D13 | A-1 | H | A-1 | m-D13 | H |
| 9919 | 0 | m-D13 | A-2 | H | A-2 | m-D13 | H |
| 9920 | 0 | m-D13 | A-3 | H | A-3 | m-D13 | H |
| 9921 | 0 | m-D13 | A-4 | H | A-4 | m-D13 | H |
| 9922 | 0 | m-D13 | A-5 | H | A-5 | m-D13 | H |
| 9923 | 0 | m-D13 | A-6 | H | A-6 | m-D13 | H |
| 9924 | 0 | m-D13 | A-7 | H | A-7 | m-D13 | H |
| 9925 | 0 | m-D13 | A-8 | H | A-8 | m-D13 | H |
| 9926 | 0 | m-D13 | A-9 | H | A-9 | m-D13 | H |
| 9927 | 0 | m-D13 | A-10 | H | A-10 | m-D13 | H |
| 9928 | 0 | m-D13 | A-11 | H | A-11 | m-D13 | H |
| 9929 | 0 | m-D13 | A-12 | H | A-12 | m-D13 | H |
| 9930 | 0 | m-D13 | A-13 | H | A-13 | m-D13 | H |
| 9931 | 0 | m-D13 | A-14 | H | A-14 | m-D13 | H |
| 9932 | 0 | m-D13 | A-15 | H | A-15 | m-D13 | H |
| 9933 | 0 | m-D13 | A-16 | H | A-16 | m-D13 | H |
| 9934 | 0 | m-D13 | A-17 | H | A-17 | m-D13 | H |
| 9935 | 0 | m-D13 | A-18 | H | A-18 | m-D13 | H |
| 9936 | 0 | m-D13 | A-19 | H | A-19 | m-D13 | H |
| 9937 | 0 | m-D13 | A-20 | H | A-20 | m-D13 | H |
| 9938 | 0 | m-D13 | A-21 | H | A-21 | m-D13 | H |
| 9939 | 0 | m-D13 | A-22 | H | A-22 | m-D13 | H |
| 9940 | 0 | m-D13 | A-23 | H | A-23 | m-D13 | H |
| 9941 | 0 | m-D13 | A-24 | H | A-24 | m-D13 | H |
| 9942 | 0 | m-D13 | A-25 | H | A-25 | m-D13 | H |
| 9943 | 0 | m-D13 | A-26 | H | A-26 | m-D13 | H |
| 9944 | 0 | m-D13 | A-27 | H | A-27 | m-D13 | H |
| 9945 | 0 | m-D13 | A-28 | H | A-28 | m-D13 | H |
| 9946 | 0 | m-D13 | A-29 | H | A-29 | m-D13 | H |
| 9947 | 0 | m-D13 | A-30 | H | A-30 | m-D13 | H |
| 9948 | 0 | m-D13 | A-31 | H | A-31 | m-D13 | H |
| 9949 | 0 | m-D13 | A-32 | H | A-32 | m-D13 | H |
| 9950 | 0 | m-D13 | A-33 | H | A-33 | m-D13 | H |
| 9951 | 0 | m-D13 | A-34 | H | A-34 | m-D13 | H |
| 9952 | 0 | m-D13 | A-35 | H | A-35 | m-D13 | H |
| 9953 | 0 | m-D13 | A-36 | H | A-36 | m-D13 | H |
| 9954 | 0 | m-D13 | A-37 | H | A-37 | m-D13 | H |
| 9955 | 0 | m-D13 | A-38 | H | A-38 | m-D13 | H |
| 9956 | 0 | m-D13 | A-39 | H | A-39 | m-D13 | H |
| 9957 | 0 | m-D13 | A-40 | H | A-40 | m-D13 | H |
| 9958 | 0 | m-D13 | A-41 | H | A-41 | m-D13 | H |
| 9959 | 0 | m-D13 | A-42 | H | A-42 | m-D13 | H |
| 9960 | 0 | m-D13 | A-43 | H | A-43 | m-D13 | H |
| 9961 | 0 | m-D13 | A-44 | H | A-44 | m-D13 | H |
| 9962 | 0 | m-D13 | A-45 | H | A-45 | m-D13 | H |
| 9963 | 0 | m-D13 | A-46 | H | A-46 | m-D13 | H |
| 9964 | 0 | m-D13 | A-47 | H | A-47 | m-D13 | H |
| 9965 | 0 | m-D14 | A-1 | H | A-1 | m-D14 | H |
| 9966 | 0 | m-D14 | A-2 | H | A-2 | m-D14 | H |
| 9967 | 0 | m-D14 | A-3 | H | A-3 | m-D14 | H |
| 9968 | 0 | m-D14 | A-4 | H | A-4 | m-D14 | H |
| 9969 | 0 | m-D14 | A-5 | H | A-5 | m-D14 | H |
| 9970 | 0 | m-D14 | A-6 | H | A-6 | m-D14 | H |
| 9971 | 0 | m-D14 | A-7 | H | A-7 | m-D14 | H |
| 9972 | 0 | m-D14 | A-8 | H | A-8 | m-D14 | H |
| 9973 | 0 | m-D14 | A-9 | H | A-9 | m-D14 | H |
| 9974 | 0 | m-D14 | A-10 | H | A-10 | m-D14 | H |
| 9975 | 0 | m-D14 | A-11 | H | A-11 | m-D14 | H |
| 9976 | 0 | m-D14 | A-12 | H | A-12 | m-D14 | H |
| 9977 | 0 | m-D14 | A-13 | H | A-13 | m-D14 | H |
| 9978 | 0 | m-D14 | A-14 | H | A-14 | m-D14 | H |
| 9979 | 0 | m-D14 | A-15 | H | A-15 | m-D14 | H |
| 9980 | 0 | m-D14 | A-16 | H | A-16 | m-D14 | H |
| 9981 | 0 | m-D14 | A-17 | H | A-17 | m-D14 | H |
| 9982 | 0 | m-D14 | A-18 | H | A-18 | m-D14 | H |
| 9983 | 0 | m-D14 | A-19 | H | A-19 | m-D14 | H |
| 9984 | 0 | m-D14 | A-20 | H | A-20 | m-D14 | H |
| 9985 | 0 | m-D14 | A-21 | H | A-21 | m-D14 | H |
| 9986 | 0 | m-D14 | A-22 | H | A-22 | m-D14 | H |
| 9987 | 0 | m-D14 | A-23 | H | A-23 | m-D14 | H |
| 9988 | 0 | m-D14 | A-24 | H | A-24 | m-D14 | H |
| 9989 | 0 | m-D14 | A-25 | H | A-25 | m-D14 | H |
| 9990 | 0 | m-D14 | A-26 | H | A-26 | m-D14 | H |
| 9991 | 0 | m-D14 | A-27 | H | A-27 | m-D14 | H |
| 9992 | 0 | m-D14 | A-28 | H | A-28 | m-D14 | H |
| 9993 | 0 | m-D14 | A-29 | H | A-29 | m-D14 | H |
| 9994 | 0 | m-D14 | A-30 | H | A-30 | m-D14 | H |
| 9995 | 0 | m-D14 | A-31 | H | A-31 | m-D14 | H |
| 9996 | 0 | m-D14 | A-32 | H | A-32 | m-D14 | H |
| 9997 | 0 | m-D14 | A-33 | H | A-33 | m-D14 | H |
| 9998 | 0 | m-D14 | A-34 | H | A-34 | m-D14 | H |
| 9999 | 0 | m-D14 | A-35 | H | A-35 | m-D14 | H |
| 10000 | 0 | m-D14 | A-36 | H | A-36 | m-D14 | H |
| 10001 | 0 | m-D14 | A-37 | H | A-37 | m-D14 | H |
| 10002 | 0 | m-D14 | A-38 | H | A-38 | m-D14 | H |
| 10003 | 0 | m-D14 | A-39 | H | A-39 | m-D14 | H |
| 10004 | 0 | m-D14 | A-40 | H | A-40 | m-D14 | H |
| 10005 | 0 | m-D14 | A-41 | H | A-41 | m-D14 | H |
| 10006 | 0 | m-D14 | A-42 | H | A-42 | m-D14 | H |
| 10007 | 0 | m-D14 | A-43 | H | A-43 | m-D14 | H |
| 10008 | 0 | m-D14 | A-44 | H | A-44 | m-D14 | H |
| 10009 | 0 | m-D14 | A-45 | H | A-45 | m-D14 | H |
| 10010 | 0 | m-D14 | A-46 | H | A-46 | m-D14 | H |
| 10011 | 0 | m-D14 | A-47 | H | A-47 | m-D14 | H |
| 10012 | 0 | m-D1 | Me | H | A-1 | H | Me |
| 10013 | 0 | m-D1 | Me | H | A-2 | H | Me |
| 10014 | 0 | m-D1 | Me | H | A-3 | H | Me |
| 10015 | 0 | m-D1 | Me | H | A-4 | H | Me |
| 10016 | 0 | m-D1 | Me | H | A-5 | H | Me |
| 10017 | 0 | m-D1 | Me | H | A-6 | H | Me |
| 10018 | 0 | m-D1 | Me | H | A-7 | H | Me |
| 10019 | 0 | m-D1 | Me | H | A-8 | H | Me |
| 10020 | 0 | m-D1 | Me | H | A-9 | H | Me |
| 10021 | 0 | m-D1 | Me | H | A-10 | H | Me |
| 10022 | 0 | m-D1 | Me | H | A-11 | H | Me |
| 10023 | 0 | m-D1 | Me | H | A-12 | H | Me |
| 10024 | 0 | m-D1 | Me | H | A-13 | H | Me |
| 10025 | 0 | m-D1 | Me | H | A-14 | H | Me |
| 10026 | 0 | m-D1 | Me | H | A-15 | H | Me |
| 10027 | 0 | m-D1 | Me | H | A-16 | H | Me |
| 10028 | 0 | m-D1 | Me | H | A-17 | H | Me |
| 10029 | 0 | m-D1 | Me | H | A-18 | H | Me |
| 10030 | 0 | m-D1 | Me | H | A-19 | H | Me |
| 10031 | 0 | m-D1 | Me | H | A-20 | H | Me |
| 10032 | 0 | m-D1 | Me | H | A-21 | H | Me |
| 10033 | 0 | m-D1 | Me | H | A-22 | H | Me |
| 10034 | 0 | m-D1 | Me | H | A-23 | H | Me |
| 10035 | 0 | m-D1 | Me | H | A-24 | H | Me |
| 10036 | 0 | m-D1 | Me | H | A-25 | H | Me |
| 10037 | 0 | m-D1 | Me | H | A-26 | H | Me |
| 10038 | 0 | m-D1 | Me | H | A-27 | H | Me |
| 10039 | 0 | m-D1 | Me | H | A-28 | H | Me |
| 10040 | 0 | m-D1 | Me | H | A-29 | H | Me |
| 10041 | 0 | m-D1 | Me | H | A-30 | H | Me |
| 10042 | 0 | m-D1 | Me | H | A-31 | H | Me |
| 10043 | 0 | m-D1 | Me | H | A-32 | H | Me |
| 10044 | 0 | m-D1 | Me | H | A-33 | H | Me |
| 10045 | 0 | m-D1 | Me | H | A-34 | H | Me |
| 10046 | 0 | m-D1 | Me | H | A-35 | H | Me |
| 10047 | 0 | m-D1 | Me | H | A-36 | H | Me |
| 10048 | 0 | m-D1 | Me | H | A-37 | H | Me |
| 10049 | 0 | m-D1 | Me | H | A-38 | H | Me |
| 10050 | 0 | m-D1 | Me | H | A-39 | H | Me |
| 10051 | 0 | m-D1 | Me | H | A-40 | H | Me |
| 10052 | 0 | m-D1 | Me | H | A-41 | H | Me |
| 10053 | 0 | m-D1 | Me | H | A-42 | H | Me |
| 10054 | 0 | m-D1 | Me | H | A-43 | H | Me |
| 10055 | 0 | m-D1 | Me | H | A-44 | H | Me |
| 10056 | 0 | m-D1 | Me | H | A-45 | H | Me |
| 10057 | 0 | m-D1 | Me | H | A-46 | H | Me |
| 10058 | 0 | m-D1 | Me | H | A-47 | H | Me |
| 10059 | 0 | m-D2 | Me | H | A-1 | H | Me |
| 10060 | 0 | m-D2 | Me | H | A-2 | H | Me |
| 10061 | 0 | m-D2 | Me | H | A-3 | H | Me |
| 10062 | 0 | m-D2 | Me | H | A-4 | H | Me |
| 10063 | 0 | m-D2 | Me | H | A-5 | H | Me |
| 10064 | 0 | m-D2 | Me | H | A-6 | H | Me |
| 10065 | 0 | m-D2 | Me | H | A-7 | H | Me |
| 10066 | 0 | m-D2 | Me | H | A-8 | H | Me |
| 10067 | 0 | m-D2 | Me | H | A-9 | H | Me |
| 10068 | 0 | m-D2 | Me | H | A-10 | H | Me |
| 10069 | 0 | m-D2 | Me | H | A-11 | H | Me |
| 10070 | 0 | m-D2 | Me | H | A-12 | H | Me |
| 10071 | 0 | m-D2 | Me | H | A-13 | H | Me |
| 10072 | 0 | m-D2 | Me | H | A-14 | H | Me |
| 10073 | 0 | m-D2 | Me | H | A-15 | H | Me |
| 10074 | 0 | m-D2 | Me | H | A-16 | H | Me |
| 10075 | 0 | m-D2 | Me | H | A-17 | H | Me |
| 10076 | 0 | m-D2 | Me | H | A-18 | H | Me |
| 10077 | 0 | m-D2 | Me | H | A-19 | H | Me |
| 10078 | 0 | m-D2 | Me | H | A-20 | H | Me |
| 10079 | 0 | m-D2 | Me | H | A-21 | H | Me |
| 10080 | 0 | m-D2 | Me | H | A-22 | H | Me |
| 10081 | 0 | m-D2 | Me | H | A-23 | H | Me |
| 10082 | 0 | m-D2 | Me | H | A-24 | H | Me |
| 10083 | 0 | m-D2 | Me | H | A-25 | H | Me |
| 10084 | 0 | m-D2 | Me | H | A-26 | H | Me |
| 10085 | 0 | m-D2 | Me | H | A-27 | H | Me |
| 10086 | 0 | m-D2 | Me | H | A-28 | H | Me |
| 10087 | 0 | m-D2 | Me | H | A-29 | H | Me |
| 10088 | 0 | m-D2 | Me | H | A-30 | H | Me |
| 10089 | 0 | m-D2 | Me | H | A-31 | H | Me |
| 10090 | 0 | m-D2 | Me | H | A-32 | H | Me |
| 10091 | 0 | m-D2 | Me | H | A-33 | H | Me |
| 10092 | 0 | m-D2 | Me | H | A-34 | H | Me |
| 10093 | 0 | m-D2 | Me | H | A-35 | H | Me |
| 10094 | 0 | m-D2 | Me | H | A-36 | H | Me |
| 10095 | 0 | m-D2 | Me | H | A-37 | H | Me |
| 10096 | 0 | m-D2 | Me | H | A-38 | H | Me |
| 10097 | 0 | m-D2 | Me | H | A-39 | H | Me |
| 10098 | 0 | m-D2 | Me | H | A-40 | H | Me |
| 10099 | 0 | m-D2 | Me | H | A-41 | H | Me |
| 10100 | 0 | m-D2 | Me | H | A-42 | H | Me |
| 10101 | 0 | m-D2 | Me | H | A-43 | H | Me |
| 10102 | 0 | m-D2 | Me | H | A-44 | H | Me |
| 10103 | 0 | m-D2 | Me | H | A-45 | H | Me |
| 10104 | 0 | m-D2 | Me | H | A-46 | H | Me |
| 10105 | 0 | m-D2 | Me | H | A-47 | H | Me |
| 10106 | 0 | m-D3 | Me | H | A-1 | H | Me |
| 10107 | 0 | m-D3 | Me | H | A-2 | H | Me |
| 10108 | 0 | m-D3 | Me | H | A-3 | H | Me |
| 10109 | 0 | m-D3 | Me | H | A-4 | H | Me |
| 10110 | 0 | m-D3 | Me | H | A-5 | H | Me |
| 10111 | 0 | m-D3 | Me | H | A-6 | H | Me |
| 10112 | 0 | m-D3 | Me | H | A-7 | H | Me |
| 10113 | 0 | m-D3 | Me | H | A-8 | H | Me |
| 10114 | 0 | m-D3 | Me | H | A-9 | H | Me |
| 10115 | 0 | m-D3 | Me | H | A-10 | H | Me |
| 10116 | 0 | m-D3 | Me | H | A-11 | H | Me |
| 10117 | 0 | m-D3 | Me | H | A-12 | H | Me |
| 10118 | 0 | m-D3 | Me | H | A-13 | H | Me |
| 10119 | 0 | m-D3 | Me | H | A-14 | H | Me |
| 10120 | 0 | m-D3 | Me | H | A-15 | H | Me |
| 10121 | 0 | m-D3 | Me | H | A-16 | H | Me |
| 10122 | 0 | m-D3 | Me | H | A-17 | H | Me |
| 10123 | 0 | m-D3 | Me | H | A-18 | H | Me |
| 10124 | 0 | m-D3 | Me | H | A-19 | H | Me |
| 10125 | 0 | m-D3 | Me | H | A-20 | H | Me |
| 10126 | 0 | m-D3 | Me | H | A-21 | H | Me |
| 10127 | 0 | m-D3 | Me | H | A-22 | H | Me |
| 10128 | 0 | m-D3 | Me | H | A-23 | H | Me |
| 10129 | 0 | m-D3 | Me | H | A-24 | H | Me |
| 10130 | 0 | m-D3 | Me | H | A-25 | H | Me |
| 10131 | 0 | m-D3 | Me | H | A-26 | H | Me |
| 10132 | 0 | m-D3 | Me | H | A-27 | H | Me |
| 10133 | 0 | m-D3 | Me | H | A-28 | H | Me |
| 10134 | 0 | m-D3 | Me | H | A-29 | H | Me |
| 10135 | 0 | m-D3 | Me | H | A-30 | H | Me |
| 10136 | 0 | m-D3 | Me | H | A-31 | H | Me |
| 10137 | 0 | m-D3 | Me | H | A-32 | H | Me |
| 10138 | 0 | m-D3 | Me | H | A-33 | H | Me |
| 10139 | 0 | m-D3 | Me | H | A-34 | H | Me |
| 10140 | 0 | m-D3 | Me | H | A-35 | H | Me |
| 10141 | 0 | m-D3 | Me | H | A-36 | H | Me |
| 10142 | 0 | m-D3 | Me | H | A-37 | H | Me |
| 10143 | 0 | m-D3 | Me | H | A-38 | H | Me |
| 10144 | 0 | m-D3 | Me | H | A-39 | H | Me |
| 10145 | 0 | m-D3 | Me | H | A-40 | H | Me |
| 10146 | 0 | m-D3 | Me | H | A-41 | H | Me |
| 10147 | 0 | m-D3 | Me | H | A-42 | H | Me |
| 10148 | 0 | m-D3 | Me | H | A-43 | H | Me |
| 10149 | 0 | m-D3 | Me | H | A-44 | H | Me |
| 10150 | 0 | m-D3 | Me | H | A-45 | H | Me |
| 10151 | 0 | m-D3 | Me | H | A-46 | H | Me |
| 10152 | 0 | m-D3 | Me | H | A-47 | H | Me |
| 10153 | 0 | m-D4 | Me | H | A-1 | H | Me |
| 10154 | 0 | m-D4 | Me | H | A-2 | H | Me |
| 10155 | 0 | m-D4 | Me | H | A-3 | H | Me |
| 10156 | 0 | m-D4 | Me | H | A-4 | H | Me |
| 10157 | 0 | m-D4 | Me | H | A-5 | H | Me |
| 10158 | 0 | m-D4 | Me | H | A-6 | H | Me |
| 10159 | 0 | m-D4 | Me | H | A-7 | H | Me |
| 10160 | 0 | m-D4 | Me | H | A-8 | H | Me |
| 10161 | 0 | m-D4 | Me | H | A-9 | H | Me |
| 10162 | 0 | m-D4 | Me | H | A-10 | H | Me |
| 10163 | 0 | m-D4 | Me | H | A-11 | H | Me |
| 10164 | 0 | m-D4 | Me | H | A-12 | H | Me |
| 10165 | 0 | m-D4 | Me | H | A-13 | H | Me |
| 10166 | 0 | m-D4 | Me | H | A-14 | H | Me |
| 10167 | 0 | m-D4 | Me | H | A-15 | H | Me |
| 10168 | 0 | m-D4 | Me | H | A-16 | H | Me |
| 10169 | 0 | m-D4 | Me | H | A-17 | H | Me |
| 10170 | 0 | m-D4 | Me | H | A-18 | H | Me |
| 10171 | 0 | m-D4 | Me | H | A-19 | H | Me |
| 10172 | 0 | m-D4 | Me | H | A-20 | H | Me |
| 10173 | 0 | m-D4 | Me | H | A-21 | H | Me |
| 10174 | 0 | m-D4 | Me | H | A-22 | H | Me |
| 10175 | 0 | m-D4 | Me | H | A-23 | H | Me |
| 10176 | 0 | m-D4 | Me | H | A-24 | H | Me |
| 10177 | 0 | m-D4 | Me | H | A-25 | H | Me |
| 10178 | 0 | m-D4 | Me | H | A-26 | H | Me |
| 10179 | 0 | m-D4 | Me | H | A-27 | H | Me |
| 10180 | 0 | m-D4 | Me | H | A-28 | H | Me |
| 10181 | 0 | m-D4 | Me | H | A-29 | H | Me |
| 10182 | 0 | m-D4 | Me | H | A-30 | H | Me |
| 10183 | 0 | m-D4 | Me | H | A-31 | H | Me |
| 10184 | 0 | m-D4 | Me | H | A-32 | H | Me |
| 10185 | 0 | m-D4 | Me | H | A-33 | H | Me |
| 10186 | 0 | m-D4 | Me | H | A-34 | H | Me |
| 10187 | 0 | m-D4 | Me | H | A-35 | H | Me |
| 10188 | 0 | m-D4 | Me | H | A-36 | H | Me |
| 10189 | 0 | m-D4 | Me | H | A-37 | H | Me |
| 10190 | 0 | m-D4 | Me | H | A-38 | H | Me |
| 10191 | 0 | m-D4 | Me | H | A-39 | H | Me |
| 10192 | 0 | m-D4 | Me | H | A-40 | H | Me |
| 10193 | 0 | m-D4 | Me | H | A-41 | H | Me |
| 10194 | 0 | m-D4 | Me | H | A-42 | H | Me |
| 10195 | 0 | m-D4 | Me | H | A-43 | H | Me |
| 10196 | 0 | m-D4 | Me | H | A-44 | H | Me |
| 10197 | 0 | m-D4 | Me | H | A-45 | H | Me |
| 10198 | 0 | m-D4 | Me | H | A-46 | H | Me |
| 10199 | 0 | m-D4 | Me | H | A-47 | H | Me |
| 10200 | 0 | m-D5 | Me | H | A-1 | H | Me |
| 10201 | 0 | m-D5 | Me | H | A-2 | H | Me |
| 10202 | 0 | m-D5 | Me | H | A-3 | H | Me |
| 10203 | 0 | m-D5 | Me | H | A-4 | H | Me |
| 10204 | 0 | m-D5 | Me | H | A-5 | H | Me |
| 10205 | 0 | m-D5 | Me | H | A-6 | H | Me |
| 10206 | 0 | m-D5 | Me | H | A-7 | H | Me |
| 10207 | 0 | m-D5 | Me | H | A-8 | H | Me |
| 10208 | 0 | m-D5 | Me | H | A-9 | H | Me |
| 10209 | 0 | m-D5 | Me | H | A-10 | H | Me |
| 10210 | 0 | m-D5 | Me | H | A-11 | H | Me |
| 10211 | 0 | m-D5 | Me | H | A-12 | H | Me |
| 10212 | 0 | m-D5 | Me | H | A-13 | H | Me |
| 10213 | 0 | m-D5 | Me | H | A-14 | H | Me |
| 10214 | 0 | m-D5 | Me | H | A-15 | H | Me |
| 10215 | 0 | m-D5 | Me | H | A-16 | H | Me |
| 10216 | 0 | m-D5 | Me | H | A-17 | H | Me |
| 10217 | 0 | m-D5 | Me | H | A-18 | H | Me |
| 10218 | 0 | m-D5 | Me | H | A-19 | H | Me |
| 10219 | 0 | m-D5 | Me | H | A-20 | H | Me |
| 10220 | 0 | m-D5 | Me | H | A-21 | H | Me |
| 10221 | 0 | m-D5 | Me | H | A-22 | H | Me |
| 10222 | 0 | m-D5 | Me | H | A-23 | H | Me |
| 10223 | 0 | m-D5 | Me | H | A-24 | H | Me |
| 10224 | 0 | m-D5 | Me | H | A-25 | H | Me |
| 10225 | 0 | m-D5 | Me | H | A-26 | H | Me |
| 10226 | 0 | m-D5 | Me | H | A-27 | H | Me |
| 10227 | 0 | m-D5 | Me | H | A-28 | H | Me |
| 10228 | 0 | m-D5 | Me | H | A-29 | H | Me |
| 10229 | 0 | m-D5 | Me | H | A-30 | H | Me |
| 10230 | 0 | m-D5 | Me | H | A-31 | H | Me |
| 10231 | 0 | m-D5 | Me | H | A-32 | H | Me |
| 10232 | 0 | m-D5 | Me | H | A-33 | H | Me |
| 10233 | 0 | m-D5 | Me | H | A-34 | H | Me |
| 10234 | 0 | m-D5 | Me | H | A-35 | H | Me |
| 10235 | 0 | m-D5 | Me | H | A-36 | H | Me |
| 10236 | 0 | m-D5 | Me | H | A-37 | H | Me |
| 10237 | 0 | m-D5 | Me | H | A-38 | H | Me |
| 10238 | 0 | m-D5 | Me | H | A-39 | H | Me |
| 10239 | 0 | m-D5 | Me | H | A-40 | H | Me |
| 10240 | 0 | m-D5 | Me | H | A-41 | H | Me |
| 10241 | 0 | m-D5 | Me | H | A-42 | H | Me |
| 10242 | 0 | m-D5 | Me | H | A-43 | H | Me |
| 10243 | 0 | m-D5 | Me | H | A-44 | H | Me |
| 10244 | 0 | m-D5 | Me | H | A-45 | H | Me |
| 10245 | 0 | m-D5 | Me | H | A-46 | H | Me |
| 10246 | 0 | m-D5 | Me | H | A-47 | H | Me |
| 10247 | 0 | m-D6 | Me | H | A-1 | H | Me |
| 10248 | 0 | m-D6 | Me | H | A-2 | H | Me |
| 10249 | 0 | m-D6 | Me | H | A-3 | H | Me |
| 10250 | 0 | m-D6 | Me | H | A-4 | H | Me |
| 10251 | 0 | m-D6 | Me | H | A-5 | H | Me |
| 10252 | 0 | m-D6 | Me | H | A-6 | H | Me |
| 10253 | 0 | m-D6 | Me | H | A-7 | H | Me |
| 10254 | 0 | m-D6 | Me | H | A-8 | H | Me |
| 10255 | 0 | m-D6 | Me | H | A-9 | H | Me |
| 10256 | 0 | m-D6 | Me | H | A-10 | H | Me |
| 10257 | 0 | m-D6 | Me | H | A-11 | H | Me |
| 10258 | 0 | m-D6 | Me | H | A-12 | H | Me |
| 10259 | 0 | m-D6 | Me | H | A-13 | H | Me |
| 10260 | 0 | m-D6 | Me | H | A-14 | H | Me |
| 10261 | 0 | m-D6 | Me | H | A-15 | H | Me |
| 10262 | 0 | m-D6 | Me | H | A-16 | H | Me |
| 10263 | 0 | m-D6 | Me | H | A-17 | H | Me |
| 10264 | 0 | m-D6 | Me | H | A-18 | H | Me |
| 10265 | 0 | m-D6 | Me | H | A-19 | H | Me |
| 10266 | 0 | m-D6 | Me | H | A-20 | H | Me |
| 10267 | 0 | m-D6 | Me | H | A-21 | H | Me |
| 10268 | 0 | m-D6 | Me | H | A-22 | H | Me |
| 10269 | 0 | m-D6 | Me | H | A-23 | H | Me |
| 10270 | 0 | m-D6 | Me | H | A-24 | H | Me |
| 10271 | 0 | m-D6 | Me | H | A-25 | H | Me |
| 10272 | 0 | m-D6 | Me | H | A-26 | H | Me |
| 10273 | 0 | m-D6 | Me | H | A-27 | H | Me |
| 10274 | 0 | m-D6 | Me | H | A-28 | H | Me |
| 10275 | 0 | m-D6 | Me | H | A-29 | H | Me |
| 10276 | 0 | m-D6 | Me | H | A-30 | H | Me |
| 10277 | 0 | m-D6 | Me | H | A-31 | H | Me |
| 10278 | 0 | m-D6 | Me | H | A-32 | H | Me |
| 10279 | 0 | m-D6 | Me | H | A-33 | H | Me |
| 10280 | 0 | m-D6 | Me | H | A-34 | H | Me |
| 10281 | 0 | m-D6 | Me | H | A-35 | H | Me |
| 10282 | 0 | m-D6 | Me | H | A-36 | H | Me |
| 10283 | 0 | m-D6 | Me | H | A-37 | H | Me |
| 10284 | 0 | m-D6 | Me | H | A-38 | H | Me |
| 10285 | 0 | m-D6 | Me | H | A-39 | H | Me |
| 10286 | 0 | m-D6 | Me | H | A-40 | H | Me |
| 10287 | 0 | m-D6 | Me | H | A-41 | H | Me |
| 10288 | 0 | m-D6 | Me | H | A-42 | H | Me |
| 10289 | 0 | m-D6 | Me | H | A-43 | H | Me |
| 10290 | 0 | m-D6 | Me | H | A-44 | H | Me |
| 10291 | 0 | m-D6 | Me | H | A-45 | H | Me |
| 10292 | 0 | m-D6 | Me | H | A-46 | H | Me |
| 10293 | 0 | m-D6 | Me | H | A-47 | H | Me |
| 10294 | 0 | m-D7 | Me | H | A-1 | H | Me |
| 10295 | 0 | m-D7 | Me | H | A-2 | H | Me |
| 10296 | 0 | m-D7 | Me | H | A-3 | H | Me |
| 10297 | 0 | m-D7 | Me | H | A-4 | H | Me |
| 10298 | 0 | m-D7 | Me | H | A-5 | H | Me |
| 10299 | 0 | m-D7 | Me | H | A-6 | H | Me |
| 10300 | 0 | m-D7 | Me | H | A-7 | H | Me |
| 10301 | 0 | m-D7 | Me | H | A-8 | H | Me |
| 10302 | 0 | m-D7 | Me | H | A-9 | H | Me |
| 10303 | 0 | m-D7 | Me | H | A-10 | H | Me |
| 10304 | 0 | m-D7 | Me | H | A-11 | H | Me |
| 10305 | 0 | m-D7 | Me | H | A-12 | H | Me |
| 10306 | 0 | m-D7 | Me | H | A-13 | H | Me |
| 10307 | 0 | m-D7 | Me | H | A-14 | H | Me |
| 10308 | 0 | m-D7 | Me | H | A-15 | H | Me |
| 10309 | 0 | m-D7 | Me | H | A-16 | H | Me |
| 10310 | 0 | m-D7 | Me | H | A-17 | H | Me |
| 10311 | 0 | m-D7 | Me | H | A-18 | H | Me |
| 10312 | 0 | m-D7 | Me | H | A-19 | H | Me |
| 10313 | 0 | m-D7 | Me | H | A-20 | H | Me |
| 10314 | 0 | m-D7 | Me | H | A-21 | H | Me |
| 10315 | 0 | m-D7 | Me | H | A-22 | H | Me |
| 10316 | 0 | m-D7 | Me | H | A-23 | H | Me |
| 10317 | 0 | m-D7 | Me | H | A-24 | H | Me |
| 10318 | 0 | m-D7 | Me | H | A-25 | H | Me |
| 10319 | 0 | m-D7 | Me | H | A-26 | H | Me |
| 10320 | 0 | m-D7 | Me | H | A-27 | H | Me |
| 10321 | 0 | m-D7 | Me | H | A-28 | H | Me |
| 10322 | 0 | m-D7 | Me | H | A-29 | H | Me |
| 10323 | 0 | m-D7 | Me | H | A-30 | H | Me |
| 10324 | 0 | m-D7 | Me | H | A-31 | H | Me |
| 10325 | 0 | m-D7 | Me | H | A-32 | H | Me |
| 10326 | 0 | m-D7 | Me | H | A-33 | H | Me |
| 10327 | 0 | m-D7 | Me | H | A-34 | H | Me |
| 10328 | 0 | m-D7 | Me | H | A-35 | H | Me |
| 10329 | 0 | m-D7 | Me | H | A-36 | H | Me |
| 10330 | 0 | m-D7 | Me | H | A-37 | H | Me |
| 10331 | 0 | m-D7 | Me | H | A-38 | H | Me |
| 10332 | 0 | m-D7 | Me | H | A-39 | H | Me |
| 10333 | 0 | m-D7 | Me | H | A-40 | H | Me |
| 10334 | 0 | m-D7 | Me | H | A-41 | H | Me |
| 10335 | 0 | m-D7 | Me | H | A-42 | H | Me |
| 10336 | 0 | m-D7 | Me | H | A-43 | H | Me |
| 10337 | 0 | m-D7 | Me | H | A-44 | H | Me |
| 10338 | 0 | m-D7 | Me | H | A-45 | H | Me |
| 10339 | 0 | m-D7 | Me | H | A-46 | H | Me |
| 10340 | 0 | m-D7 | Me | H | A-47 | H | Me |
| 10341 | 0 | m-D8 | Me | H | A-1 | H | Me |
| 10342 | 0 | m-D8 | Me | H | A-2 | H | Me |
| 10343 | 0 | m-D8 | Me | H | A-3 | H | Me |
| 10344 | 0 | m-D8 | Me | H | A-4 | H | Me |
| 10345 | 0 | m-D8 | Me | H | A-5 | H | Me |
| 10346 | 0 | m-D8 | Me | H | A-6 | H | Me |
| 10347 | 0 | m-D8 | Me | H | A-7 | H | Me |
| 10348 | 0 | m-D8 | Me | H | A-8 | H | Me |
| 10349 | 0 | m-D8 | Me | H | A-9 | H | Me |
| 10350 | 0 | m-D8 | Me | H | A-10 | H | Me |
| 10351 | 0 | m-D8 | Me | H | A-11 | H | Me |
| 10352 | 0 | m-D8 | Me | H | A-12 | H | Me |
| 10353 | 0 | m-D8 | Me | H | A-13 | H | Me |
| 10354 | 0 | m-D8 | Me | H | A-14 | H | Me |
| 10355 | 0 | m-D8 | Me | H | A-15 | H | Me |
| 10356 | 0 | m-D8 | Me | H | A-16 | H | Me |
| 10357 | 0 | m-D8 | Me | H | A-17 | H | Me |
| 10358 | 0 | m-D8 | Me | H | A-18 | H | Me |
| 10359 | 0 | m-D8 | Me | H | A-19 | H | Me |
| 10360 | 0 | m-D8 | Me | H | A-20 | H | Me |
| 10361 | 0 | m-D8 | Me | H | A-21 | H | Me |
| 10362 | 0 | m-D8 | Me | H | A-22 | H | Me |
| 10363 | 0 | m-D8 | Me | H | A-23 | H | Me |
| 10364 | 0 | m-D8 | Me | H | A-24 | H | Me |
| 10365 | 0 | m-D8 | Me | H | A-25 | H | Me |
| 10366 | 0 | m-D8 | Me | H | A-26 | H | Me |
| 10367 | 0 | m-D8 | Me | H | A-27 | H | Me |
| 10368 | 0 | m-D8 | Me | H | A-28 | H | Me |
| 10369 | 0 | m-D8 | Me | H | A-29 | H | Me |
| 10370 | 0 | m-D8 | Me | H | A-30 | H | Me |
| 10371 | 0 | m-D8 | Me | H | A-31 | H | Me |
| 10372 | 0 | m-D8 | Me | H | A-32 | H | Me |
| 10373 | 0 | m-D8 | Me | H | A-33 | H | Me |
| 10374 | 0 | m-D8 | Me | H | A-34 | H | Me |
| 10375 | 0 | m-D8 | Me | H | A-35 | H | Me |
| 10376 | 0 | m-D8 | Me | H | A-36 | H | Me |
| 10377 | 0 | m-D8 | Me | H | A-37 | H | Me |
| 10378 | 0 | m-D8 | Me | H | A-38 | H | Me |
| 10379 | 0 | m-D8 | Me | H | A-39 | H | Me |
| 10380 | 0 | m-D8 | Me | H | A-40 | H | Me |
| 10381 | 0 | m-D8 | Me | H | A-41 | H | Me |
| 10382 | 0 | m-D8 | Me | H | A-42 | H | Me |
| 10383 | 0 | m-D8 | Me | H | A-43 | H | Me |
| 10384 | 0 | m-D8 | Me | H | A-44 | H | Me |
| 10385 | 0 | m-D8 | Me | H | A-45 | H | Me |
| 10386 | 0 | m-D8 | Me | H | A-46 | H | Me |
| 10387 | 0 | m-D8 | Me | H | A-47 | H | Me |
| 10388 | 0 | m-D9 | Me | H | A-1 | H | Me |
| 10389 | 0 | m-D9 | Me | H | A-2 | H | Me |
| 10390 | 0 | m-D9 | Me | H | A-3 | H | Me |
| 10391 | 0 | m-D9 | Me | H | A-4 | H | Me |
| 10392 | 0 | m-D9 | Me | H | A-5 | H | Me |
| 10393 | 0 | m-D9 | Me | H | A-6 | H | Me |
| 10394 | 0 | m-D9 | Me | H | A-7 | H | Me |
| 10395 | 0 | m-D9 | Me | H | A-8 | H | Me |
| 10396 | 0 | m-D9 | Me | H | A-9 | H | Me |
| 10397 | 0 | m-D9 | Me | H | A-10 | H | Me |
| 10398 | 0 | m-D9 | Me | H | A-11 | H | Me |
| 10399 | 0 | m-D9 | Me | H | A-12 | H | Me |
| 10400 | 0 | m-D9 | Me | H | A-13 | H | Me |
| 10401 | 0 | m-D9 | Me | H | A-14 | H | Me |
| 10402 | 0 | m-D9 | Me | H | A-15 | H | Me |
| 10403 | 0 | m-D9 | Me | H | A-16 | H | Me |
| 10404 | 0 | m-D9 | Me | H | A-17 | H | Me |
| 10405 | 0 | m-D9 | Me | H | A-18 | H | Me |
| 10406 | 0 | m-D9 | Me | H | A-19 | H | Me |
| 10407 | 0 | m-D9 | Me | H | A-20 | H | Me |
| 10408 | 0 | m-D9 | Me | H | A-21 | H | Me |
| 10409 | 0 | m-D9 | Me | H | A-22 | H | Me |
| 10410 | 0 | m-D9 | Me | H | A-23 | H | Me |
| 10411 | 0 | m-D9 | Me | H | A-24 | H | Me |
| 10412 | 0 | m-D9 | Me | H | A-25 | H | Me |
| 10413 | 0 | m-D9 | Me | H | A-26 | H | Me |
| 10414 | 0 | m-D9 | Me | H | A-27 | H | Me |
| 10415 | 0 | m-D9 | Me | H | A-28 | H | Me |
| 10416 | 0 | m-D9 | Me | H | A-29 | H | Me |
| 10417 | 0 | m-D9 | Me | H | A-30 | H | Me |
| 10418 | 0 | m-D9 | Me | H | A-31 | H | Me |
| 10419 | 0 | m-D9 | Me | H | A-32 | H | Me |
| 10420 | 0 | m-D9 | Me | H | A-33 | H | Me |
| 10421 | 0 | m-D9 | Me | H | A-34 | H | Me |
| 10422 | 0 | m-D9 | Me | H | A-35 | H | Me |
| 10423 | 0 | m-D9 | Me | H | A-36 | H | Me |
| 10424 | 0 | m-D9 | Me | H | A-37 | H | Me |
| 10425 | 0 | m-D9 | Me | H | A-38 | H | Me |
| 10426 | 0 | m-D9 | Me | H | A-39 | H | Me |
| 10427 | 0 | m-D9 | Me | H | A-40 | H | Me |
| 10428 | 0 | m-D9 | Me | H | A-41 | H | Me |
| 10429 | 0 | m-D9 | Me | H | A-42 | H | Me |
| 10430 | 0 | m-D9 | Me | H | A-43 | H | Me |
| 10431 | 0 | m-D9 | Me | H | A-44 | H | Me |
| 10432 | 0 | m-D9 | Me | H | A-45 | H | Me |
| 10433 | 0 | m-D9 | Me | H | A-46 | H | Me |
| 10434 | 0 | m-D9 | Me | H | A-47 | H | Me |
| 10435 | 0 | m-D10 | Me | H | A-1 | H | Me |
| 10436 | 0 | m-D10 | Me | H | A-2 | H | Me |
| 10437 | 0 | m-D10 | Me | H | A-3 | H | Me |
| 10438 | 0 | m-D10 | Me | H | A-4 | H | Me |
| 10439 | 0 | m-D10 | Me | H | A-5 | H | Me |
| 10440 | 0 | m-D10 | Me | H | A-6 | H | Me |
| 10441 | 0 | m-D10 | Me | H | A-7 | H | Me |
| 10442 | 0 | m-D10 | Me | H | A-8 | H | Me |
| 10443 | 0 | m-D10 | Me | H | A-9 | H | Me |
| 10444 | 0 | m-D10 | Me | H | A-10 | H | Me |
| 10445 | 0 | m-D10 | Me | H | A-11 | H | Me |
| 10446 | 0 | m-D10 | Me | H | A-12 | H | Me |
| 10447 | 0 | m-D10 | Me | H | A-13 | H | Me |
| 10448 | 0 | m-D10 | Me | H | A-14 | H | Me |
| 10449 | 0 | m-D10 | Me | H | A-15 | H | Me |
| 10450 | 0 | m-D10 | Me | H | A-16 | H | Me |
| 10451 | 0 | m-D10 | Me | H | A-17 | H | Me |
| 10452 | 0 | m-D10 | Me | H | A-18 | H | Me |
| 10453 | 0 | m-D10 | Me | H | A-19 | H | Me |
| 10454 | 0 | m-D10 | Me | H | A-20 | H | Me |
| 10455 | 0 | m-D10 | Me | H | A-21 | H | Me |
| 10456 | 0 | m-D10 | Me | H | A-22 | H | Me |
| 10457 | 0 | m-D10 | Me | H | A-23 | H | Me |
| 10458 | 0 | m-D10 | Me | H | A-24 | H | Me |
| 10459 | 0 | m-D10 | Me | H | A-25 | H | Me |
| 10460 | 0 | m-D10 | Me | H | A-26 | H | Me |
| 10461 | 0 | m-D10 | Me | H | A-27 | H | Me |
| 10462 | 0 | m-D10 | Me | H | A-28 | H | Me |
| 10463 | 0 | m-D10 | Me | H | A-29 | H | Me |
| 10464 | 0 | m-D10 | Me | H | A-30 | H | Me |
| 10465 | 0 | m-D10 | Me | H | A-31 | H | Me |
| 10466 | 0 | m-D10 | Me | H | A-32 | H | Me |
| 10467 | 0 | m-D10 | Me | H | A-33 | H | Me |
| 10468 | 0 | m-D10 | Me | H | A-34 | H | Me |
| 10469 | 0 | m-D10 | Me | H | A-35 | H | Me |
| 10470 | 0 | m-D10 | Me | H | A-36 | H | Me |
| 10471 | 0 | m-D10 | Me | H | A-37 | H | Me |
| 10472 | 0 | m-D10 | Me | H | A-38 | H | Me |
| 10473 | 0 | m-D10 | Me | H | A-39 | H | Me |
| 10474 | 0 | m-D10 | Me | H | A-40 | H | Me |
| 10475 | 0 | m-D10 | Me | H | A-41 | H | Me |
| 10476 | 0 | m-D10 | Me | H | A-42 | H | Me |
| 10477 | 0 | m-D10 | Me | H | A-43 | H | Me |
| 10478 | 0 | m-D10 | Me | H | A-44 | H | Me |
| 10479 | 0 | m-D10 | Me | H | A-45 | H | Me |
| 10480 | 0 | m-D10 | Me | H | A-46 | H | Me |
| 10481 | 0 | m-D10 | Me | H | A-47 | H | Me |
| 10482 | 0 | m-D11 | Me | H | A-1 | H | Me |
| 10483 | 0 | m-D11 | Me | H | A-2 | H | Me |
| 10484 | 0 | m-D11 | Me | H | A-3 | H | Me |
| 10485 | 0 | m-D11 | Me | H | A-4 | H | Me |
| 10486 | 0 | m-D11 | Me | H | A-5 | H | Me |
| 10487 | 0 | m-D11 | Me | H | A-6 | H | Me |
| 10488 | 0 | m-D11 | Me | H | A-7 | H | Me |
| 10489 | 0 | m-D11 | Me | H | A-8 | H | Me |
| 10490 | 0 | m-D11 | Me | H | A-9 | H | Me |
| 10491 | 0 | m-D11 | Me | H | A-10 | H | Me |
| 10492 | 0 | m-D11 | Me | H | A-11 | H | Me |
| 10493 | 0 | m-D11 | Me | H | A-12 | H | Me |
| 10494 | 0 | m-D11 | Me | H | A-13 | H | Me |
| 10495 | 0 | m-D11 | Me | H | A-14 | H | Me |
| 10496 | 0 | m-D11 | Me | H | A-15 | H | Me |
| 10497 | 0 | m-D11 | Me | H | A-16 | H | Me |
| 10498 | 0 | m-D11 | Me | H | A-17 | H | Me |
| 10499 | 0 | m-D11 | Me | H | A-18 | H | Me |
| 10500 | 0 | m-D11 | Me | H | A-19 | H | Me |
| 10501 | 0 | m-D11 | Me | H | A-20 | H | Me |
| 10502 | 0 | m-D11 | Me | H | A-21 | H | Me |
| 10503 | 0 | m-D11 | Me | H | A-22 | H | Me |
| 10504 | 0 | m-D11 | Me | H | A-23 | H | Me |
| 10505 | 0 | m-D11 | Me | H | A-24 | H | Me |
| 10506 | 0 | m-D11 | Me | H | A-25 | H | Me |
| 10507 | 0 | m-D11 | Me | H | A-26 | H | Me |
| 10508 | 0 | m-D11 | Me | H | A-27 | H | Me |
| 10509 | 0 | m-D11 | Me | H | A-28 | H | Me |
| 10510 | 0 | m-D11 | Me | H | A-29 | H | Me |
| 10511 | 0 | m-D11 | Me | H | A-30 | H | Me |
| 10512 | 0 | m-D11 | Me | H | A-31 | H | Me |
| 10513 | 0 | m-D11 | Me | H | A-32 | H | Me |
| 10514 | 0 | m-D11 | Me | H | A-33 | H | Me |
| 10515 | 0 | m-D11 | Me | H | A-34 | H | Me |
| 10516 | 0 | m-D11 | Me | H | A-35 | H | Me |
| 10517 | 0 | m-D11 | Me | H | A-36 | H | Me |
| 10518 | 0 | m-D11 | Me | H | A-37 | H | Me |
| 10519 | 0 | m-D11 | Me | H | A-38 | H | Me |
| 10520 | 0 | m-D11 | Me | H | A-39 | H | Me |
| 10521 | 0 | m-D11 | Me | H | A-40 | H | Me |
| 10522 | 0 | m-D11 | Me | H | A-41 | H | Me |
| 10523 | 0 | m-D11 | Me | H | A-42 | H | Me |
| 10524 | 0 | m-D11 | Me | H | A-43 | H | Me |
| 10525 | 0 | m-D11 | Me | H | A-44 | H | Me |
| 10526 | 0 | m-D11 | Me | H | A-45 | H | Me |
| 10527 | 0 | m-D11 | Me | H | A-46 | H | Me |
| 10528 | 0 | m-D11 | Me | H | A-47 | H | Me |
| 10529 | 0 | m-D12 | Me | H | A-1 | H | Me |
| 10530 | 0 | m-D12 | Me | H | A-2 | H | Me |
| 10531 | 0 | m-D12 | Me | H | A-3 | H | Me |
| 10532 | 0 | m-D12 | Me | H | A-4 | H | Me |
| 10533 | 0 | m-D12 | Me | H | A-5 | H | Me |
| 10534 | 0 | m-D12 | Me | H | A-6 | H | Me |
| 10535 | 0 | m-D12 | Me | H | A-7 | H | Me |
| 10536 | 0 | m-D12 | Me | H | A-8 | H | Me |
| 10537 | 0 | m-D12 | Me | H | A-9 | H | Me |
| 10538 | 0 | m-D12 | Me | H | A-10 | H | Me |
| 10539 | 0 | m-D12 | Me | H | A-11 | H | Me |
| 10540 | 0 | m-D12 | Me | H | A-12 | H | Me |
| 10541 | 0 | m-D12 | Me | H | A-13 | H | Me |
| 10542 | 0 | m-D12 | Me | H | A-14 | H | Me |
| 10543 | 0 | m-D12 | Me | H | A-15 | H | Me |
| 10544 | 0 | m-D12 | Me | H | A-16 | H | Me |
| 10545 | 0 | m-D12 | Me | H | A-17 | H | Me |
| 10546 | 0 | m-D12 | Me | H | A-18 | H | Me |
| 10547 | 0 | m-D12 | Me | H | A-19 | H | Me |
| 10548 | 0 | m-D12 | Me | H | A-20 | H | Me |
| 10549 | 0 | m-D12 | Me | H | A-21 | H | Me |
| 10550 | 0 | m-D12 | Me | H | A-22 | H | Me |
| 10551 | 0 | m-D12 | Me | H | A-23 | H | Me |
| 10552 | 0 | m-D12 | Me | H | A-24 | H | Me |
| 10553 | 0 | m-D12 | Me | H | A-25 | H | Me |
| 10554 | 0 | m-D12 | Me | H | A-26 | H | Me |
| 10555 | 0 | m-D12 | Me | H | A-27 | H | Me |
| 10556 | 0 | m-D12 | Me | H | A-28 | H | Me |
| 10557 | 0 | m-D12 | Me | H | A-29 | H | Me |
| 10558 | 0 | m-D12 | Me | H | A-30 | H | Me |
| 10559 | 0 | m-D12 | Me | H | A-31 | H | Me |
| 10560 | 0 | m-D12 | Me | H | A-32 | H | Me |
| 10561 | 0 | m-D12 | Me | H | A-33 | H | Me |
| 10562 | 0 | m-D12 | Me | H | A-34 | H | Me |
| 10563 | 0 | m-D12 | Me | H | A-35 | H | Me |
| 10564 | 0 | m-D12 | Me | H | A-36 | H | Me |
| 10565 | 0 | m-D12 | Me | H | A-37 | H | Me |
| 10566 | 0 | m-D12 | Me | H | A-38 | H | Me |
| 10567 | 0 | m-D12 | Me | H | A-39 | H | Me |
| 10568 | 0 | m-D12 | Me | H | A-40 | H | Me |
| 10569 | 0 | m-D12 | Me | H | A-41 | H | Me |
| 10570 | 0 | m-D12 | Me | H | A-42 | H | Me |
| 10571 | 0 | m-D12 | Me | H | A-43 | H | Me |
| 10572 | 0 | m-D12 | Me | H | A-44 | H | Me |
| 10573 | 0 | m-D12 | Me | H | A-45 | H | Me |
| 10574 | 0 | m-D12 | Me | H | A-46 | H | Me |
| 10575 | 0 | m-D12 | Me | H | A-47 | H | Me |
| 10576 | 0 | m-D13 | Me | H | A-1 | H | Me |
| 10577 | 0 | m-D13 | Me | H | A-2 | H | Me |
| 10578 | 0 | m-D13 | Me | H | A-3 | H | Me |
| 10579 | 0 | m-D13 | Me | H | A-4 | H | Me |
| 10580 | 0 | m-D13 | Me | H | A-5 | H | Me |
| 10581 | 0 | m-D13 | Me | H | A-6 | H | Me |
| 10582 | 0 | m-D13 | Me | H | A-7 | H | Me |
| 10583 | 0 | m-D13 | Me | H | A-8 | H | Me |
| 10584 | 0 | m-D13 | Me | H | A-9 | H | Me |
| 10585 | 0 | m-D13 | Me | H | A-10 | H | Me |
| 10586 | 0 | m-D13 | Me | H | A-11 | H | Me |
| 10587 | 0 | m-D13 | Me | H | A-12 | H | Me |
| 10588 | 0 | m-D13 | Me | H | A-13 | H | Me |
| 10589 | 0 | m-D13 | Me | H | A-14 | H | Me |
| 10590 | 0 | m-D13 | Me | H | A-15 | H | Me |
| 10591 | 0 | m-D13 | Me | H | A-16 | H | Me |
| 10592 | 0 | m-D13 | Me | H | A-17 | H | Me |
| 10593 | 0 | m-D13 | Me | H | A-18 | H | Me |
| 10594 | 0 | m-D13 | Me | H | A-19 | H | Me |
| 10595 | 0 | m-D13 | Me | H | A-20 | H | Me |
| 10596 | 0 | m-D13 | Me | H | A-21 | H | Me |
| 10597 | 0 | m-D13 | Me | H | A-22 | H | Me |
| 10598 | 0 | m-D13 | Me | H | A-23 | H | Me |
| 10599 | 0 | m-D13 | Me | H | A-24 | H | Me |
| 10600 | 0 | m-D13 | Me | H | A-25 | H | Me |
| 10601 | 0 | m-D13 | Me | H | A-26 | H | Me |
| 10602 | 0 | m-D13 | Me | H | A-27 | H | Me |
| 10603 | 0 | m-D13 | Me | H | A-28 | H | Me |
| 10604 | 0 | m-D13 | Me | H | A-29 | H | Me |
| 10605 | 0 | m-D13 | Me | H | A-30 | H | Me |
| 10606 | 0 | m-D13 | Me | H | A-31 | H | Me |
| 10607 | 0 | m-D13 | Me | H | A-32 | H | Me |
| 10608 | 0 | m-D13 | Me | H | A-33 | H | Me |
| 10609 | 0 | m-D13 | Me | H | A-34 | H | Me |
| 10610 | 0 | m-D13 | Me | H | A-35 | H | Me |
| 10611 | 0 | m-D13 | Me | H | A-36 | H | Me |
| 10612 | 0 | m-D13 | Me | H | A-37 | H | Me |
| 10613 | 0 | m-D13 | Me | H | A-38 | H | Me |
| 10614 | 0 | m-D13 | Me | H | A-39 | H | Me |
| 10615 | 0 | m-D13 | Me | H | A-40 | H | Me |
| 10616 | 0 | m-D13 | Me | H | A-41 | H | Me |
| 10617 | 0 | m-D13 | Me | H | A-42 | H | Me |
| 10618 | 0 | m-D13 | Me | H | A-43 | H | Me |
| 10619 | 0 | m-D13 | Me | H | A-44 | H | Me |
| 10620 | 0 | m-D13 | Me | H | A-45 | H | Me |
| 10621 | 0 | m-D13 | Me | H | A-46 | H | Me |
| 10622 | 0 | m-D13 | Me | H | A-47 | H | Me |
| 10623 | 0 | m-D14 | Me | H | A-1 | H | Me |
| 10624 | 0 | m-D14 | Me | H | A-2 | H | Me |
| 10625 | 0 | m-D14 | Me | H | A-3 | H | Me |
| 10626 | 0 | m-D14 | Me | H | A-4 | H | Me |
| 10627 | 0 | m-D14 | Me | H | A-5 | H | Me |
| 10628 | 0 | m-D14 | Me | H | A-6 | H | Me |
| 10629 | 0 | m-D14 | Me | H | A-7 | H | Me |
| 10630 | 0 | m-D14 | Me | H | A-8 | H | Me |
| 10631 | 0 | m-D14 | Me | H | A-9 | H | Me |
| 10632 | 0 | m-D14 | Me | H | A-10 | H | Me |
| 10633 | 0 | m-D14 | Me | H | A-11 | H | Me |
| 10634 | 0 | m-D14 | Me | H | A-12 | H | Me |
| 10635 | 0 | m-D14 | Me | H | A-13 | H | Me |
| 10636 | 0 | m-D14 | Me | H | A-14 | H | Me |
| 10637 | 0 | m-D14 | Me | H | A-15 | H | Me |
| 10638 | 0 | m-D14 | Me | H | A-16 | H | Me |
| 10639 | 0 | m-D14 | Me | H | A-17 | H | Me |
| 10640 | 0 | m-D14 | Me | H | A-18 | H | Me |
| 10641 | 0 | m-D14 | Me | H | A-19 | H | Me |
| 10642 | 0 | m-D14 | Me | H | A-20 | H | Me |
| 10643 | 0 | m-D14 | Me | H | A-21 | H | Me |
| 10644 | 0 | m-D14 | Me | H | A-22 | H | Me |
| 10645 | 0 | m-D14 | Me | H | A-23 | H | Me |
| 10646 | 0 | m-D14 | Me | H | A-24 | H | Me |
| 10647 | 0 | m-D14 | Me | H | A-25 | H | Me |
| 10648 | 0 | m-D14 | Me | H | A-26 | H | Me |
| 10649 | 0 | m-D14 | Me | H | A-27 | H | Me |
| 10650 | 0 | m-D14 | Me | H | A-28 | H | Me |
| 10651 | 0 | m-D14 | Me | H | A-29 | H | Me |
| 10652 | 0 | m-D14 | Me | H | A-30 | H | Me |
| 10653 | 0 | m-D14 | Me | H | A-31 | H | Me |
| 10654 | 0 | m-D14 | Me | H | A-32 | H | Me |
| 10655 | 0 | m-D14 | Me | H | A-33 | H | Me |
| 10656 | 0 | m-D14 | Me | H | A-34 | H | Me |
| 10657 | 0 | m-D14 | Me | H | A-35 | H | Me |
| 10658 | 0 | m-D14 | Me | H | A-36 | H | Me |
| 10659 | 0 | m-D14 | Me | H | A-37 | H | Me |
| 10660 | 0 | m-D14 | Me | H | A-38 | H | Me |
| 10661 | 0 | m-D14 | Me | H | A-39 | H | Me |
| 10662 | 0 | m-D14 | Me | H | A-40 | H | Me |
| 10663 | 0 | m-D14 | Me | H | A-41 | H | Me |
| 10664 | 0 | m-D14 | Me | H | A-42 | H | Me |
| 10665 | 0 | m-D14 | Me | H | A-43 | H | Me |
| 10666 | 0 | m-D14 | Me | H | A-44 | H | Me |
| 10667 | 0 | m-D14 | Me | H | A-45 | H | Me |
| 10668 | 0 | m-D14 | Me | H | A-46 | H | Me |
| 10669 | 0 | m-D14 | Me | H | A-47 | H | Me |
| 10670 | 0 | m-D1 | H | Me | A-1 | Me | H |
| 10671 | 0 | m-D1 | H | Me | A-2 | Me | H |
| 10672 | 0 | m-D1 | H | Me | A-3 | Me | H |
| 10673 | 0 | m-D1 | H | Me | A-4 | Me | H |
| 10674 | 0 | m-D1 | H | Me | A-5 | Me | H |
| 10675 | 0 | m-D1 | H | Me | A-6 | Me | H |
| 10676 | 0 | m-D1 | H | Me | A-7 | Me | H |
| 10677 | 0 | m-D1 | H | Me | A-8 | Me | H |
| 10678 | 0 | m-D1 | H | Me | A-9 | Me | H |
| 10679 | 0 | m-D1 | H | Me | A-10 | Me | H |
| 10680 | 0 | m-D1 | H | Me | A-11 | Me | H |
| 10681 | 0 | m-D1 | H | Me | A-12 | Me | H |
| 10682 | 0 | m-D1 | H | Me | A-13 | Me | H |
| 10683 | 0 | m-D1 | H | Me | A-14 | Me | H |
| 10684 | 0 | m-D1 | H | Me | A-15 | Me | H |
| 10685 | 0 | m-D1 | H | Me | A-16 | Me | H |
| 10686 | 0 | m-D1 | H | Me | A-17 | Me | H |
| 10687 | 0 | m-D1 | H | Me | A-18 | Me | H |
| 10688 | 0 | m-D1 | H | Me | A-19 | Me | H |
| 10689 | 0 | m-D1 | H | Me | A-20 | Me | H |
| 10690 | 0 | m-D1 | H | Me | A-21 | Me | H |
| 10691 | 0 | m-D1 | H | Me | A-22 | Me | H |
| 10692 | 0 | m-D1 | H | Me | A-23 | Me | H |
| 10693 | 0 | m-D1 | H | Me | A-24 | Me | H |
| 10694 | 0 | m-D1 | H | Me | A-25 | Me | H |
| 10695 | 0 | m-D1 | H | Me | A-26 | Me | H |
| 10696 | 0 | m-D1 | H | Me | A-27 | Me | H |
| 10697 | 0 | m-D1 | H | Me | A-28 | Me | H |
| 10698 | 0 | m-D1 | H | Me | A-29 | Me | H |
| 10699 | 0 | m-D1 | H | Me | A-30 | Me | H |
| 10700 | 0 | m-D1 | H | Me | A-31 | Me | H |
| 10701 | 0 | m-D1 | H | Me | A-32 | Me | H |
| 10702 | 0 | m-D1 | H | Me | A-33 | Me | H |
| 10703 | 0 | m-D1 | H | Me | A-34 | Me | H |
| 10704 | 0 | m-D1 | H | Me | A-35 | Me | H |
| 10705 | 0 | m-D1 | H | Me | A-36 | Me | H |
| 10706 | 0 | m-D1 | H | Me | A-37 | Me | H |
| 10707 | 0 | m-D1 | H | Me | A-38 | Me | H |
| 10708 | 0 | m-D1 | H | Me | A-39 | Me | H |
| 10709 | 0 | m-D1 | H | Me | A-40 | Me | H |
| 10710 | 0 | m-D1 | H | Me | A-41 | Me | H |
| 10711 | 0 | m-D1 | H | Me | A-42 | Me | H |
| 10712 | 0 | m-D1 | H | Me | A-43 | Me | H |
| 10713 | 0 | m-D1 | H | Me | A-44 | Me | H |
| 10714 | 0 | m-D1 | H | Me | A-45 | Me | H |
| 10715 | 0 | m-D1 | H | Me | A-46 | Me | H |
| 10716 | 0 | m-D1 | H | Me | A-47 | Me | H |
| 10717 | 0 | m-D2 | H | Me | A-1 | Me | H |
| 10718 | 0 | m-D2 | H | Me | A-2 | Me | H |
| 10719 | 0 | m-D2 | H | Me | A-3 | Me | H |
| 10720 | 0 | m-D2 | H | Me | A-4 | Me | H |
| 10721 | 0 | m-D2 | H | Me | A-5 | Me | H |
| 10722 | 0 | m-D2 | H | Me | A-6 | Me | H |
| 10723 | 0 | m-D2 | H | Me | A-7 | Me | H |
| 10724 | 0 | m-D2 | H | Me | A-8 | Me | H |
| 10725 | 0 | m-D2 | H | Me | A-9 | Me | H |
| 10726 | 0 | m-D2 | H | Me | A-10 | Me | H |
| 10727 | 0 | m-D2 | H | Me | A-11 | Me | H |
| 10728 | 0 | m-D2 | H | Me | A-12 | Me | H |
| 10729 | 0 | m-D2 | H | Me | A-13 | Me | H |
| 10730 | 0 | m-D2 | H | Me | A-14 | Me | H |
| 10731 | 0 | m-D2 | H | Me | A-15 | Me | H |
| 10732 | 0 | m-D2 | H | Me | A-16 | Me | H |
| 10733 | 0 | m-D2 | H | Me | A-17 | Me | H |
| 10734 | 0 | m-D2 | H | Me | A-18 | Me | H |
| 10735 | 0 | m-D2 | H | Me | A-19 | Me | H |
| 10736 | 0 | m-D2 | H | Me | A-20 | Me | H |
| 10737 | 0 | m-D2 | H | Me | A-21 | Me | H |
| 10738 | 0 | m-D2 | H | Me | A-22 | Me | H |
| 10739 | 0 | m-D2 | H | Me | A-23 | Me | H |
| 10740 | 0 | m-D2 | H | Me | A-24 | Me | H |
| 10741 | 0 | m-D2 | H | Me | A-25 | Me | H |
| 10742 | 0 | m-D2 | H | Me | A-26 | Me | H |
| 10743 | 0 | m-D2 | H | Me | A-27 | Me | H |
| 10744 | 0 | m-D2 | H | Me | A-28 | Me | H |
| 10745 | 0 | m-D2 | H | Me | A-29 | Me | H |
| 10746 | 0 | m-D2 | H | Me | A-30 | Me | H |
| 10747 | 0 | m-D2 | H | Me | A-31 | Me | H |
| 10748 | 0 | m-D2 | H | Me | A-32 | Me | H |
| 10749 | 0 | m-D2 | H | Me | A-33 | Me | H |
| 10750 | 0 | m-D2 | H | Me | A-34 | Me | H |
| 10751 | 0 | m-D2 | H | Me | A-35 | Me | H |
| 10752 | 0 | m-D2 | H | Me | A-36 | Me | H |
| 10753 | 0 | m-D2 | H | Me | A-37 | Me | H |
| 10754 | 0 | m-D2 | H | Me | A-38 | Me | H |
| 10755 | 0 | m-D2 | H | Me | A-39 | Me | H |
| 10756 | 0 | m-D2 | H | Me | A-40 | Me | H |
| 10757 | 0 | m-D2 | H | Me | A-41 | Me | H |
| 10758 | 0 | m-D2 | H | Me | A-42 | Me | H |
| 10759 | 0 | m-D2 | H | Me | A-43 | Me | H |
| 10760 | 0 | m-D2 | H | Me | A-44 | Me | H |
| 10761 | 0 | m-D2 | H | Me | A-45 | Me | H |
| 10762 | 0 | m-D2 | H | Me | A-46 | Me | H |
| 10763 | 0 | m-D2 | H | Me | A-47 | Me | H |
| 10764 | 0 | m-D3 | H | Me | A-1 | Me | H |
| 10765 | 0 | m-D3 | H | Me | A-2 | Me | H |
| 10766 | 0 | m-D3 | H | Me | A-3 | Me | H |
| 10767 | 0 | m-D3 | H | Me | A-4 | Me | H |
| 10768 | 0 | m-D3 | H | Me | A-5 | Me | H |
| 10769 | 0 | m-D3 | H | Me | A-6 | Me | H |
| 10770 | 0 | m-D3 | H | Me | A-7 | Me | H |
| 10771 | 0 | m-D3 | H | Me | A-8 | Me | H |
| 10772 | 0 | m-D3 | H | Me | A-9 | Me | H |
| 10773 | 0 | m-D3 | H | Me | A-10 | Me | H |
| 10774 | 0 | m-D3 | H | Me | A-11 | Me | H |
| 10775 | 0 | m-D3 | H | Me | A-12 | Me | H |
| 10776 | 0 | m-D3 | H | Me | A-13 | Me | H |
| 10777 | 0 | m-D3 | H | Me | A-14 | Me | H |
| 10778 | 0 | m-D3 | H | Me | A-15 | Me | H |
| 10779 | 0 | m-D3 | H | Me | A-16 | Me | H |
| 10780 | 0 | m-D3 | H | Me | A-17 | Me | H |
| 10781 | 0 | m-D3 | H | Me | A-18 | Me | H |
| 10782 | 0 | m-D3 | H | Me | A-19 | Me | H |
| 10783 | 0 | m-D3 | H | Me | A-20 | Me | H |
| 10784 | 0 | m-D3 | H | Me | A-21 | Me | H |
| 10785 | 0 | m-D3 | H | Me | A-22 | Me | H |
| 10786 | 0 | m-D3 | H | Me | A-23 | Me | H |
| 10787 | 0 | m-D3 | H | Me | A-24 | Me | H |
| 10788 | 0 | m-D3 | H | Me | A-25 | Me | H |
| 10789 | 0 | m-D3 | H | Me | A-26 | Me | H |
| 10790 | 0 | m-D3 | H | Me | A-27 | Me | H |
| 10791 | 0 | m-D3 | H | Me | A-28 | Me | H |
| 10792 | 0 | m-D3 | H | Me | A-29 | Me | H |
| 10793 | 0 | m-D3 | H | Me | A-30 | Me | H |
| 10794 | 0 | m-D3 | H | Me | A-31 | Me | H |
| 10795 | 0 | m-D3 | H | Me | A-32 | Me | H |
| 10796 | 0 | m-D3 | H | Me | A-33 | Me | H |
| 10797 | 0 | m-D3 | H | Me | A-34 | Me | H |
| 10798 | 0 | m-D3 | H | Me | A-35 | Me | H |
| 10799 | 0 | m-D3 | H | Me | A-36 | Me | H |
| 10800 | 0 | m-D3 | H | Me | A-37 | Me | H |
| 10801 | 0 | m-D3 | H | Me | A-38 | Me | H |
| 10802 | 0 | m-D3 | H | Me | A-39 | Me | H |
| 10803 | 0 | m-D3 | H | Me | A-40 | Me | H |
| 10804 | 0 | m-D3 | H | Me | A-41 | Me | H |
| 10805 | 0 | m-D3 | H | Me | A-42 | Me | H |
| 10806 | 0 | m-D3 | H | Me | A-43 | Me | H |
| 10807 | 0 | m-D3 | H | Me | A-44 | Me | H |
| 10808 | 0 | m-D3 | H | Me | A-45 | Me | H |
| 10809 | 0 | m-D3 | H | Me | A-46 | Me | H |
| 10810 | 0 | m-D3 | H | Me | A-47 | Me | H |
| 10811 | 0 | m-D4 | H | Me | A-1 | Me | H |
| 10812 | 0 | m-D4 | H | Me | A-2 | Me | H |
| 10813 | 0 | m-D4 | H | Me | A-3 | Me | H |
| 10814 | 0 | m-D4 | H | Me | A-4 | Me | H |
| 10815 | 0 | m-D4 | H | Me | A-5 | Me | H |
| 10816 | 0 | m-D4 | H | Me | A-6 | Me | H |
| 10817 | 0 | m-D4 | H | Me | A-7 | Me | H |
| 10818 | 0 | m-D4 | H | Me | A-8 | Me | H |
| 10819 | 0 | m-D4 | H | Me | A-9 | Me | H |
| 10820 | 0 | m-D4 | H | Me | A-10 | Me | H |
| 10821 | 0 | m-D4 | H | Me | A-11 | Me | H |
| 10822 | 0 | m-D4 | H | Me | A-12 | Me | H |
| 10823 | 0 | m-D4 | H | Me | A-13 | Me | H |
| 10824 | 0 | m-D4 | H | Me | A-14 | Me | H |
| 10825 | 0 | m-D4 | H | Me | A-15 | Me | H |
| 10826 | 0 | m-D4 | H | Me | A-16 | Me | H |
| 10827 | 0 | m-D4 | H | Me | A-17 | Me | H |
| 10828 | 0 | m-D4 | H | Me | A-18 | Me | H |
| 10829 | 0 | m-D4 | H | Me | A-19 | Me | H |
| 10830 | 0 | m-D4 | H | Me | A-20 | Me | H |
| 10831 | 0 | m-D4 | H | Me | A-21 | Me | H |
| 10832 | 0 | m-D4 | H | Me | A-22 | Me | H |
| 10833 | 0 | m-D4 | H | Me | A-23 | Me | H |
| 10834 | 0 | m-D4 | H | Me | A-24 | Me | H |
| 10835 | 0 | m-D4 | H | Me | A-25 | Me | H |
| 10836 | 0 | m-D4 | H | Me | A-26 | Me | H |
| 10837 | 0 | m-D4 | H | Me | A-27 | Me | H |
| 10838 | 0 | m-D4 | H | Me | A-28 | Me | H |
| 10839 | 0 | m-D4 | H | Me | A-29 | Me | H |
| 10840 | 0 | m-D4 | H | Me | A-30 | Me | H |
| 10841 | 0 | m-D4 | H | Me | A-31 | Me | H |
| 10842 | 0 | m-D4 | H | Me | A-32 | Me | H |
| 10843 | 0 | m-D4 | H | Me | A-33 | Me | H |
| 10844 | 0 | m-D4 | H | Me | A-34 | Me | H |
| 10845 | 0 | m-D4 | H | Me | A-35 | Me | H |
| 10846 | 0 | m-D4 | H | Me | A-36 | Me | H |
| 10847 | 0 | m-D4 | H | Me | A-37 | Me | H |
| 10848 | 0 | m-D4 | H | Me | A-38 | Me | H |
| 10849 | 0 | m-D4 | H | Me | A-39 | Me | H |
| 10850 | 0 | m-D4 | H | Me | A-40 | Me | H |
| 10851 | 0 | m-D4 | H | Me | A-41 | Me | H |
| 10852 | 0 | m-D4 | H | Me | A-42 | Me | H |
| 10853 | 0 | m-D4 | H | Me | A-43 | Me | H |
| 10854 | 0 | m-D4 | H | Me | A-44 | Me | H |
| 10855 | 0 | m-D4 | H | Me | A-45 | Me | H |
| 10856 | 0 | m-D4 | H | Me | A-46 | Me | H |
| 10857 | 0 | m-D4 | H | Me | A-47 | Me | H |
| 10858 | 0 | m-D5 | H | Me | A-1 | Me | H |
| 10859 | 0 | m-D5 | H | Me | A-2 | Me | H |
| 10860 | 0 | m-D5 | H | Me | A-3 | Me | H |
| 10861 | 0 | m-D5 | H | Me | A-4 | Me | H |
| 10862 | 0 | m-D5 | H | Me | A-5 | Me | H |
| 10863 | 0 | m-D5 | H | Me | A-6 | Me | H |
| 10864 | 0 | m-D5 | H | Me | A-7 | Me | H |
| 10865 | 0 | m-D5 | H | Me | A-8 | Me | H |
| 10866 | 0 | m-D5 | H | Me | A-9 | Me | H |
| 10867 | 0 | m-D5 | H | Me | A-10 | Me | H |
| 10868 | 0 | m-D5 | H | Me | A-11 | Me | H |
| 10869 | 0 | m-D5 | H | Me | A-12 | Me | H |
| 10870 | 0 | m-D5 | H | Me | A-13 | Me | H |
| 10871 | 0 | m-D5 | H | Me | A-14 | Me | H |
| 10872 | 0 | m-D5 | H | Me | A-15 | Me | H |
| 10873 | 0 | m-D5 | H | Me | A-16 | Me | H |
| 10874 | 0 | m-D5 | H | Me | A-17 | Me | H |
| 10875 | 0 | m-D5 | H | Me | A-18 | Me | H |
| 10876 | 0 | m-D5 | H | Me | A-19 | Me | H |
| 10877 | 0 | m-D5 | H | Me | A-20 | Me | H |
| 10878 | 0 | m-D5 | H | Me | A-21 | Me | H |
| 10879 | 0 | m-D5 | H | Me | A-22 | Me | H |
| 10880 | 0 | m-D5 | H | Me | A-23 | Me | H |
| 10881 | 0 | m-D5 | H | Me | A-24 | Me | H |
| 10882 | 0 | m-D5 | H | Me | A-25 | Me | H |
| 10883 | 0 | m-D5 | H | Me | A-26 | Me | H |
| 10884 | 0 | m-D5 | H | Me | A-27 | Me | H |
| 10885 | 0 | m-D5 | H | Me | A-28 | Me | H |
| 10886 | 0 | m-D5 | H | Me | A-29 | Me | H |
| 10887 | 0 | m-D5 | H | Me | A-30 | Me | H |
| 10888 | 0 | m-D5 | H | Me | A-31 | Me | H |
| 10889 | 0 | m-D5 | H | Me | A-32 | Me | H |
| 10890 | 0 | m-D5 | H | Me | A-33 | Me | H |
| 10891 | 0 | m-D5 | H | Me | A-34 | Me | H |
| 10892 | 0 | m-D5 | H | Me | A-35 | Me | H |
| 10893 | 0 | m-D5 | H | Me | A-36 | Me | H |
| 10894 | 0 | m-D5 | H | Me | A-37 | Me | H |
| 10895 | 0 | m-D5 | H | Me | A-38 | Me | H |
| 10896 | 0 | m-D5 | H | Me | A-39 | Me | H |
| 10897 | 0 | m-D5 | H | Me | A-40 | Me | H |
| 10898 | 0 | m-D5 | H | Me | A-41 | Me | H |
| 10899 | 0 | m-D5 | H | Me | A-42 | Me | H |
| 10900 | 0 | m-D5 | H | Me | A-43 | Me | H |
| 10901 | 0 | m-D5 | H | Me | A-44 | Me | H |
| 10902 | 0 | m-D5 | H | Me | A-45 | Me | H |
| 10903 | 0 | m-D5 | H | Me | A-46 | Me | H |
| 10904 | 0 | m-D5 | H | Me | A-47 | Me | H |
| 10905 | 0 | m-D6 | H | Me | A-1 | Me | H |
| 10906 | 0 | m-D6 | H | Me | A-2 | Me | H |
| 10907 | 0 | m-D6 | H | Me | A-3 | Me | H |
| 10908 | 0 | m-D6 | H | Me | A-4 | Me | H |
| 10909 | 0 | m-D6 | H | Me | A-5 | Me | H |
| 10910 | 0 | m-D6 | H | Me | A-6 | Me | H |
| 10911 | 0 | m-D6 | H | Me | A-7 | Me | H |
| 10912 | 0 | m-D6 | H | Me | A-8 | Me | H |
| 10913 | 0 | m-D6 | H | Me | A-9 | Me | H |
| 10914 | 0 | m-D6 | H | Me | A-10 | Me | H |
| 10915 | 0 | m-D6 | H | Me | A-11 | Me | H |
| 10916 | 0 | m-D6 | H | Me | A-12 | Me | H |
| 10917 | 0 | m-D6 | H | Me | A-13 | Me | H |
| 10918 | 0 | m-D6 | H | Me | A-14 | Me | H |
| 10919 | 0 | m-D6 | H | Me | A-15 | Me | H |
| 10920 | 0 | m-D6 | H | Me | A-16 | Me | H |
| 10921 | 0 | m-D6 | H | Me | A-17 | Me | H |
| 10922 | 0 | m-D6 | H | Me | A-18 | Me | H |
| 10923 | 0 | m-D6 | H | Me | A-19 | Me | H |
| 10924 | 0 | m-D6 | H | Me | A-20 | Me | H |
| 10925 | 0 | m-D6 | H | Me | A-21 | Me | H |
| 10926 | 0 | m-D6 | H | Me | A-22 | Me | H |
| 10927 | 0 | m-D6 | H | Me | A-23 | Me | H |
| 10928 | 0 | m-D6 | H | Me | A-24 | Me | H |
| 10929 | 0 | m-D6 | H | Me | A-25 | Me | H |
| 10930 | 0 | m-D6 | H | Me | A-26 | Me | H |
| 10931 | 0 | m-D6 | H | Me | A-27 | Me | H |
| 10932 | 0 | m-D6 | H | Me | A-28 | Me | H |
| 10933 | 0 | m-D6 | H | Me | A-29 | Me | H |
| 10934 | 0 | m-D6 | H | Me | A-30 | Me | H |
| 10935 | 0 | m-D6 | H | Me | A-31 | Me | H |
| 10936 | 0 | m-D6 | H | Me | A-32 | Me | H |
| 10937 | 0 | m-D6 | H | Me | A-33 | Me | H |
| 10938 | 0 | m-D6 | H | Me | A-34 | Me | H |
| 10939 | 0 | m-D6 | H | Me | A-35 | Me | H |
| 10940 | 0 | m-D6 | H | Me | A-36 | Me | H |
| 10941 | 0 | m-D6 | H | Me | A-37 | Me | H |
| 10942 | 0 | m-D6 | H | Me | A-38 | Me | H |
| 10943 | 0 | m-D6 | H | Me | A-39 | Me | H |
| 10944 | 0 | m-D6 | H | Me | A-40 | Me | H |
| 10945 | 0 | m-D6 | H | Me | A-41 | Me | H |
| 10946 | 0 | m-D6 | H | Me | A-42 | Me | H |
| 10947 | 0 | m-D6 | H | Me | A-43 | Me | H |
| 10948 | 0 | m-D6 | H | Me | A-44 | Me | H |
| 10949 | 0 | m-D6 | H | Me | A-45 | Me | H |
| 10950 | 0 | m-D6 | H | Me | A-46 | Me | H |
| 10951 | 0 | m-D6 | H | Me | A-47 | Me | H |
| 10952 | 0 | m-D7 | H | Me | A-1 | Me | H |
| 10953 | 0 | m-D7 | H | Me | A-2 | Me | H |
| 10954 | 0 | m-D7 | H | Me | A-3 | Me | H |
| 10955 | 0 | m-D7 | H | Me | A-4 | Me | H |
| 10956 | 0 | m-D7 | H | Me | A-5 | Me | H |
| 10957 | 0 | m-D7 | H | Me | A-6 | Me | H |
| 10958 | 0 | m-D7 | H | Me | A-7 | Me | H |
| 10959 | 0 | m-D7 | H | Me | A-8 | Me | H |
| 10960 | 0 | m-D7 | H | Me | A-9 | Me | H |
| 10961 | 0 | m-D7 | H | Me | A-10 | Me | H |
| 10962 | 0 | m-D7 | H | Me | A-11 | Me | H |
| 10963 | 0 | m-D7 | H | Me | A-12 | Me | H |
| 10964 | 0 | m-D7 | H | Me | A-13 | Me | H |
| 10965 | 0 | m-D7 | H | Me | A-14 | Me | H |
| 10966 | 0 | m-D7 | H | Me | A-15 | Me | H |
| 10967 | 0 | m-D7 | H | Me | A-16 | Me | H |
| 10968 | 0 | m-D7 | H | Me | A-17 | Me | H |
| 10969 | 0 | m-D7 | H | Me | A-18 | Me | H |
| 10970 | 0 | m-D7 | H | Me | A-19 | Me | H |
| 10971 | 0 | m-D7 | H | Me | A-20 | Me | H |
| 10972 | 0 | m-D7 | H | Me | A-21 | Me | H |
| 10973 | 0 | m-D7 | H | Me | A-22 | Me | H |
| 10974 | 0 | m-D7 | H | Me | A-23 | Me | H |
| 10975 | 0 | m-D7 | H | Me | A-24 | Me | H |
| 10976 | 0 | m-D7 | H | Me | A-25 | Me | H |
| 10977 | 0 | m-D7 | H | Me | A-26 | Me | H |
| 10978 | 0 | m-D7 | H | Me | A-27 | Me | H |
| 10979 | 0 | m-D7 | H | Me | A-28 | Me | H |
| 10980 | 0 | m-D7 | H | Me | A-29 | Me | H |
| 10981 | 0 | m-D7 | H | Me | A-30 | Me | H |
| 10982 | 0 | m-D7 | H | Me | A-31 | Me | H |
| 10983 | 0 | m-D7 | H | Me | A-32 | Me | H |
| 10984 | 0 | m-D7 | H | Me | A-33 | Me | H |
| 10985 | 0 | m-D7 | H | Me | A-34 | Me | H |
| 10986 | 0 | m-D7 | H | Me | A-35 | Me | H |
| 10987 | 0 | m-D7 | H | Me | A-36 | Me | H |
| 10988 | 0 | m-D7 | H | Me | A-37 | Me | H |
| 10989 | 0 | m-D7 | H | Me | A-38 | Me | H |
| 10990 | 0 | m-D7 | H | Me | A-39 | Me | H |
| 10991 | 0 | m-D7 | H | Me | A-40 | Me | H |
| 10992 | 0 | m-D7 | H | Me | A-41 | Me | H |
| 10993 | 0 | m-D7 | H | Me | A-42 | Me | H |
| 10994 | 0 | m-D7 | H | Me | A-43 | Me | H |
| 10995 | 0 | m-D7 | H | Me | A-44 | Me | H |
| 10996 | 0 | m-D7 | H | Me | A-45 | Me | H |
| 10997 | 0 | m-D7 | H | Me | A-46 | Me | H |
| 10998 | 0 | m-D7 | H | Me | A-47 | Me | H |
| 10999 | 0 | m-D8 | H | Me | A-1 | Me | H |
| 11000 | 0 | m-D8 | H | Me | A-2 | Me | H |
| 11001 | 0 | m-D8 | H | Me | A-3 | Me | H |
| 11002 | 0 | m-D8 | H | Me | A-4 | Me | H |
| 11003 | 0 | m-D8 | H | Me | A-5 | Me | H |
| 11004 | 0 | m-D8 | H | Me | A-6 | Me | H |
| 11005 | 0 | m-D8 | H | Me | A-7 | Me | H |
| 11006 | 0 | m-D8 | H | Me | A-8 | Me | H |
| 11007 | 0 | m-D8 | H | Me | A-9 | Me | H |
| 11008 | 0 | m-D8 | H | Me | A-10 | Me | H |
| 11009 | 0 | m-D8 | H | Me | A-11 | Me | H |
| 11010 | 0 | m-D8 | H | Me | A-12 | Me | H |
| 11011 | 0 | m-D8 | H | Me | A-13 | Me | H |
| 11012 | 0 | m-D8 | H | Me | A-14 | Me | H |
| 11013 | 0 | m-D8 | H | Me | A-15 | Me | H |
| 11014 | 0 | m-D8 | H | Me | A-16 | Me | H |
| 11015 | 0 | m-D8 | H | Me | A-17 | Me | H |
| 11016 | 0 | m-D8 | H | Me | A-18 | Me | H |
| 11017 | 0 | m-D8 | H | Me | A-19 | Me | H |
| 11018 | 0 | m-D8 | H | Me | A-20 | Me | H |
| 11019 | 0 | m-D8 | H | Me | A-21 | Me | H |
| 11020 | 0 | m-D8 | H | Me | A-22 | Me | H |
| 11021 | 0 | m-D8 | H | Me | A-23 | Me | H |
| 11022 | 0 | m-D8 | H | Me | A-24 | Me | H |
| 11023 | 0 | m-D8 | H | Me | A-25 | Me | H |
| 11024 | 0 | m-D8 | H | Me | A-26 | Me | H |
| 11025 | 0 | m-D8 | H | Me | A-27 | Me | H |
| 11026 | 0 | m-D8 | H | Me | A-28 | Me | H |
| 11027 | 0 | m-D8 | H | Me | A-29 | Me | H |
| 11028 | 0 | m-D8 | H | Me | A-30 | Me | H |
| 11029 | 0 | m-D8 | H | Me | A-31 | Me | H |
| 11030 | 0 | m-D8 | H | Me | A-32 | Me | H |
| 11031 | 0 | m-D8 | H | Me | A-33 | Me | H |
| 11032 | 0 | m-D8 | H | Me | A-34 | Me | H |
| 11033 | 0 | m-D8 | H | Me | A-35 | Me | H |
| 11034 | 0 | m-D8 | H | Me | A-36 | Me | H |
| 11035 | 0 | m-D8 | H | Me | A-37 | Me | H |
| 11036 | 0 | m-D8 | H | Me | A-38 | Me | H |
| 11037 | 0 | m-D8 | H | Me | A-39 | Me | H |
| 11038 | 0 | m-D8 | H | Me | A-40 | Me | H |
| 11039 | 0 | m-D8 | H | Me | A-41 | Me | H |
| 11040 | 0 | m-D8 | H | Me | A-42 | Me | H |
| 11041 | 0 | m-D8 | H | Me | A-43 | Me | H |
| 11042 | 0 | m-D8 | H | Me | A-44 | Me | H |
| 11043 | 0 | m-D8 | H | Me | A-45 | Me | H |
| 11044 | 0 | m-D8 | H | Me | A-46 | Me | H |
| 11045 | 0 | m-D8 | H | Me | A-47 | Me | H |
| 11046 | 0 | m-D9 | H | Me | A-1 | Me | H |
| 11047 | 0 | m-D9 | H | Me | A-2 | Me | H |
| 11048 | 0 | m-D9 | H | Me | A-3 | Me | H |
| 11049 | 0 | m-D9 | H | Me | A-4 | Me | H |
| 11050 | 0 | m-D9 | H | Me | A-5 | Me | H |
| 11051 | 0 | m-D9 | H | Me | A-6 | Me | H |
| 11052 | 0 | m-D9 | H | Me | A-7 | Me | H |
| 11053 | 0 | m-D9 | H | Me | A-8 | Me | H |
| 11054 | 0 | m-D9 | H | Me | A-9 | Me | H |
| 11055 | 0 | m-D9 | H | Me | A-10 | Me | H |
| 11056 | 0 | m-D9 | H | Me | A-11 | Me | H |
| 11057 | 0 | m-D9 | H | Me | A-12 | Me | H |
| 11058 | 0 | m-D9 | H | Me | A-13 | Me | H |
| 11059 | 0 | m-D9 | H | Me | A-14 | Me | H |
| 11060 | 0 | m-D9 | H | Me | A-15 | Me | H |
| 11061 | 0 | m-D9 | H | Me | A-16 | Me | H |
| 11062 | 0 | m-D9 | H | Me | A-17 | Me | H |
| 11063 | 0 | m-D9 | H | Me | A-18 | Me | H |
| 11064 | 0 | m-D9 | H | Me | A-19 | Me | H |
| 11065 | 0 | m-D9 | H | Me | A-20 | Me | H |
| 11066 | 0 | m-D9 | H | Me | A-21 | Me | H |
| 11067 | 0 | m-D9 | H | Me | A-22 | Me | H |
| 11068 | 0 | m-D9 | H | Me | A-23 | Me | H |
| 11069 | 0 | m-D9 | H | Me | A-24 | Me | H |
| 11070 | 0 | m-D9 | H | Me | A-25 | Me | H |
| 11071 | 0 | m-D9 | H | Me | A-26 | Me | H |
| 11072 | 0 | m-D9 | H | Me | A-27 | Me | H |
| 11073 | 0 | m-D9 | H | Me | A-28 | Me | H |
| 11074 | 0 | m-D9 | H | Me | A-29 | Me | H |
| 11075 | 0 | m-D9 | H | Me | A-30 | Me | H |
| 11076 | 0 | m-D9 | H | Me | A-31 | Me | H |
| 11077 | 0 | m-D9 | H | Me | A-32 | Me | H |
| 11078 | 0 | m-D9 | H | Me | A-33 | Me | H |
| 11079 | 0 | m-D9 | H | Me | A-34 | Me | H |
| 11080 | 0 | m-D9 | H | Me | A-35 | Me | H |
| 11081 | 0 | m-D9 | H | Me | A-36 | Me | H |
| 11082 | 0 | m-D9 | H | Me | A-37 | Me | H |
| 11083 | 0 | m-D9 | H | Me | A-38 | Me | H |
| 11084 | 0 | m-D9 | H | Me | A-39 | Me | H |
| 11085 | 0 | m-D9 | H | Me | A-40 | Me | H |
| 11086 | 0 | m-D9 | H | Me | A-41 | Me | H |
| 11087 | 0 | m-D9 | H | Me | A-42 | Me | H |
| 11088 | 0 | m-D9 | H | Me | A-43 | Me | H |
| 11089 | 0 | m-D9 | H | Me | A-44 | Me | H |
| 11090 | 0 | m-D9 | H | Me | A-45 | Me | H |
| 11091 | 0 | m-D9 | H | Me | A-46 | Me | H |
| 11092 | 0 | m-D9 | H | Me | A-47 | Me | H |
| 11093 | 0 | m-D10 | H | Me | A-1 | Me | H |
| 11094 | 0 | m-D10 | H | Me | A-2 | Me | H |
| 11095 | 0 | m-D10 | H | Me | A-3 | Me | H |
| 11096 | 0 | m-D10 | H | Me | A-4 | Me | H |
| 11097 | 0 | m-D10 | H | Me | A-5 | Me | H |
| 11098 | 0 | m-D10 | H | Me | A-6 | Me | H |
| 11099 | 0 | m-D10 | H | Me | A-7 | Me | H |
| 11100 | 0 | m-D10 | H | Me | A-8 | Me | H |
| 11101 | 0 | m-D10 | H | Me | A-9 | Me | H |
| 11102 | 0 | m-D10 | H | Me | A-10 | Me | H |
| 11103 | 0 | m-D10 | H | Me | A-11 | Me | H |
| 11104 | 0 | m-D10 | H | Me | A-12 | Me | H |
| 11105 | 0 | m-D10 | H | Me | A-13 | Me | H |
| 11106 | 0 | m-D10 | H | Me | A-14 | Me | H |
| 11107 | 0 | m-D10 | H | Me | A-15 | Me | H |
| 11108 | 0 | m-D10 | H | Me | A-16 | Me | H |
| 11109 | 0 | m-D10 | H | Me | A-17 | Me | H |
| 11110 | 0 | m-D10 | H | Me | A-18 | Me | H |
| 11111 | 0 | m-D10 | H | Me | A-19 | Me | H |
| 11112 | 0 | m-D10 | H | Me | A-20 | Me | H |
| 11113 | 0 | m-D10 | H | Me | A-21 | Me | H |
| 11114 | 0 | m-D10 | H | Me | A-22 | Me | H |
| 11115 | 0 | m-D10 | H | Me | A-23 | Me | H |
| 11116 | 0 | m-D10 | H | Me | A-24 | Me | H |
| 11117 | 0 | m-D10 | H | Me | A-25 | Me | H |
| 11118 | 0 | m-D10 | H | Me | A-26 | Me | H |
| 11119 | 0 | m-D10 | H | Me | A-27 | Me | H |
| 11120 | 0 | m-D10 | H | Me | A-28 | Me | H |
| 11121 | 0 | m-D10 | H | Me | A-29 | Me | H |
| 11122 | 0 | m-D10 | H | Me | A-30 | Me | H |
| 11123 | 0 | m-D10 | H | Me | A-31 | Me | H |
| 11124 | 0 | m-D10 | H | Me | A-32 | Me | H |
| 11125 | 0 | m-D10 | H | Me | A-33 | Me | H |
| 11126 | 0 | m-D10 | H | Me | A-34 | Me | H |
| 11127 | 0 | m-D10 | H | Me | A-35 | Me | H |
| 11128 | 0 | m-D10 | H | Me | A-36 | Me | H |
| 11129 | 0 | m-D10 | H | Me | A-37 | Me | H |
| 11130 | 0 | m-D10 | H | Me | A-38 | Me | H |
| 11131 | 0 | m-D10 | H | Me | A-39 | Me | H |
| 11132 | 0 | m-D10 | H | Me | A-40 | Me | H |
| 11133 | 0 | m-D10 | H | Me | A-41 | Me | H |
| 11134 | 0 | m-D10 | H | Me | A-42 | Me | H |
| 11135 | 0 | m-D10 | H | Me | A-43 | Me | H |
| 11136 | 0 | m-D10 | H | Me | A-44 | Me | H |
| 11137 | 0 | m-D10 | H | Me | A-45 | Me | H |
| 11138 | 0 | m-D10 | H | Me | A-46 | Me | H |
| 11139 | 0 | m-D10 | H | Me | A-47 | Me | H |
| 11140 | 0 | m-D11 | H | Me | A-1 | Me | H |
| 11141 | 0 | m-D11 | H | Me | A-2 | Me | H |
| 11142 | 0 | m-D11 | H | Me | A-3 | Me | H |
| 11143 | 0 | m-D11 | H | Me | A-4 | Me | H |
| 11144 | 0 | m-D11 | H | Me | A-5 | Me | H |
| 11145 | 0 | m-D11 | H | Me | A-6 | Me | H |
| 11146 | 0 | m-D11 | H | Me | A-7 | Me | H |
| 11147 | 0 | m-D11 | H | Me | A-8 | Me | H |
| 11148 | 0 | m-D11 | H | Me | A-9 | Me | H |
| 11149 | 0 | m-D11 | H | Me | A-10 | Me | H |
| 11150 | 0 | m-D11 | H | Me | A-11 | Me | H |
| 11151 | 0 | m-D11 | H | Me | A-12 | Me | H |
| 11152 | 0 | m-D11 | H | Me | A-13 | Me | H |
| 11153 | 0 | m-D11 | H | Me | A-14 | Me | H |
| 11154 | 0 | m-D11 | H | Me | A-15 | Me | H |
| 11155 | 0 | m-D11 | H | Me | A-16 | Me | H |
| 11156 | 0 | m-D11 | H | Me | A-17 | Me | H |
| 11157 | 0 | m-D11 | H | Me | A-18 | Me | H |
| 11158 | 0 | m-D11 | H | Me | A-19 | Me | H |
| 11159 | 0 | m-D11 | H | Me | A-20 | Me | H |
| 11160 | 0 | m-D11 | H | Me | A-21 | Me | H |
| 11161 | 0 | m-D11 | H | Me | A-22 | Me | H |
| 11162 | 0 | m-D11 | H | Me | A-23 | Me | H |
| 11163 | 0 | m-D11 | H | Me | A-24 | Me | H |
| 11164 | 0 | m-D11 | H | Me | A-25 | Me | H |
| 11165 | 0 | m-D11 | H | Me | A-26 | Me | H |
| 11166 | 0 | m-D11 | H | Me | A-27 | Me | H |
| 11167 | 0 | m-D11 | H | Me | A-28 | Me | H |
| 11168 | 0 | m-D11 | H | Me | A-29 | Me | H |
| 11169 | 0 | m-D11 | H | Me | A-30 | Me | H |
| 11170 | 0 | m-D11 | H | Me | A-31 | Me | H |
| 11171 | 0 | m-D11 | H | Me | A-32 | Me | H |
| 11172 | 0 | m-D11 | H | Me | A-33 | Me | H |
| 11173 | 0 | m-D11 | H | Me | A-34 | Me | H |
| 11174 | 0 | m-D11 | H | Me | A-35 | Me | H |
| 11175 | 0 | m-D11 | H | Me | A-36 | Me | H |
| 11176 | 0 | m-D11 | H | Me | A-37 | Me | H |
| 11177 | 0 | m-D11 | H | Me | A-38 | Me | H |
| 11178 | 0 | m-D11 | H | Me | A-39 | Me | H |
| 11179 | 0 | m-D11 | H | Me | A-40 | Me | H |
| 11180 | 0 | m-D11 | H | Me | A-41 | Me | H |
| 11181 | 0 | m-D11 | H | Me | A-42 | Me | H |
| 11182 | 0 | m-D11 | H | Me | A-43 | Me | H |
| 11183 | 0 | m-D11 | H | Me | A-44 | Me | H |
| 11184 | 0 | m-D11 | H | Me | A-45 | Me | H |
| 11185 | 0 | m-D11 | H | Me | A-46 | Me | H |
| 11186 | 0 | m-D11 | H | Me | A-47 | Me | H |
| 11187 | 0 | m-D12 | H | Me | A-1 | Me | H |
| 11188 | 0 | m-D12 | H | Me | A-2 | Me | H |
| 11189 | 0 | m-D12 | H | Me | A-3 | Me | H |
| 11190 | 0 | m-D12 | H | Me | A-4 | Me | H |
| 11191 | 0 | m-D12 | H | Me | A-5 | Me | H |
| 11192 | 0 | m-D12 | H | Me | A-6 | Me | H |
| 11193 | 0 | m-D12 | H | Me | A-7 | Me | H |
| 11194 | 0 | m-D12 | H | Me | A-8 | Me | H |
| 11195 | 0 | m-D12 | H | Me | A-9 | Me | H |
| 11196 | 0 | m-D12 | H | Me | A-10 | Me | H |
| 11197 | 0 | m-D12 | H | Me | A-11 | Me | H |
| 11198 | 0 | m-D12 | H | Me | A-12 | Me | H |
| 11199 | 0 | m-D12 | H | Me | A-13 | Me | H |
| 11200 | 0 | m-D12 | H | Me | A-14 | Me | H |
| 11201 | 0 | m-D12 | H | Me | A-15 | Me | H |
| 11202 | 0 | m-D12 | H | Me | A-16 | Me | H |
| 11203 | 0 | m-D12 | H | Me | A-17 | Me | H |
| 11204 | 0 | m-D12 | H | Me | A-18 | Me | H |
| 11205 | 0 | m-D12 | H | Me | A-19 | Me | H |
| 11206 | 0 | m-D12 | H | Me | A-20 | Me | H |
| 11207 | 0 | m-D12 | H | Me | A-21 | Me | H |
| 11208 | 0 | m-D12 | H | Me | A-22 | Me | H |
| 11209 | 0 | m-D12 | H | Me | A-23 | Me | H |
| 11210 | 0 | m-D12 | H | Me | A-24 | Me | H |
| 11211 | 0 | m-D12 | H | Me | A-25 | Me | H |
| 11212 | 0 | m-D12 | H | Me | A-26 | Me | H |
| 11213 | 0 | m-D12 | H | Me | A-27 | Me | H |
| 11214 | 0 | m-D12 | H | Me | A-28 | Me | H |
| 11215 | 0 | m-D12 | H | Me | A-29 | Me | H |
| 11216 | 0 | m-D12 | H | Me | A-30 | Me | H |
| 11217 | 0 | m-D12 | H | Me | A-31 | Me | H |
| 11218 | 0 | m-D12 | H | Me | A-32 | Me | H |
| 11219 | 0 | m-D12 | H | Me | A-33 | Me | H |
| 11220 | 0 | m-D12 | H | Me | A-34 | Me | H |
| 11221 | 0 | m-D12 | H | Me | A-35 | Me | H |
| 11222 | 0 | m-D12 | H | Me | A-36 | Me | H |
| 11223 | 0 | m-D12 | H | Me | A-37 | Me | H |
| 11224 | 0 | m-D12 | H | Me | A-38 | Me | H |
| 11225 | 0 | m-D12 | H | Me | A-39 | Me | H |
| 11226 | 0 | m-D12 | H | Me | A-40 | Me | H |
| 11227 | 0 | m-D12 | H | Me | A-41 | Me | H |
| 11228 | 0 | m-D12 | H | Me | A-42 | Me | H |
| 11229 | 0 | m-D12 | H | Me | A-43 | Me | H |
| 11230 | 0 | m-D12 | H | Me | A-44 | Me | H |
| 11231 | 0 | m-D12 | H | Me | A-45 | Me | H |
| 11232 | 0 | m-D12 | H | Me | A-46 | Me | H |
| 11233 | 0 | m-D12 | H | Me | A-47 | Me | H |
| 11234 | 0 | m-D13 | H | Me | A-1 | Me | H |
| 11235 | 0 | m-D13 | H | Me | A-2 | Me | H |
| 11236 | 0 | m-D13 | H | Me | A-3 | Me | H |
| 11237 | 0 | m-D13 | H | Me | A-4 | Me | H |
| 11238 | 0 | m-D13 | H | Me | A-5 | Me | H |
| 11239 | 0 | m-D13 | H | Me | A-6 | Me | H |
| 11240 | 0 | m-D13 | H | Me | A-7 | Me | H |
| 11241 | 0 | m-D13 | H | Me | A-8 | Me | H |
| 11242 | 0 | m-D13 | H | Me | A-9 | Me | H |
| 11243 | 0 | m-D13 | H | Me | A-10 | Me | H |
| 11244 | 0 | m-D13 | H | Me | A-11 | Me | H |
| 11245 | 0 | m-D13 | H | Me | A-12 | Me | H |
| 11246 | 0 | m-D13 | H | Me | A-13 | Me | H |
| 11247 | 0 | m-D13 | H | Me | A-14 | Me | H |
| 11248 | 0 | m-D13 | H | Me | A-15 | Me | H |
| 11249 | 0 | m-D13 | H | Me | A-16 | Me | H |
| 11250 | 0 | m-D13 | H | Me | A-17 | Me | H |
| 11251 | 0 | m-D13 | H | Me | A-18 | Me | H |
| 11252 | 0 | m-D13 | H | Me | A-19 | Me | H |
| 11253 | 0 | m-D13 | H | Me | A-20 | Me | H |
| 11254 | 0 | m-D13 | H | Me | A-21 | Me | H |
| 11255 | 0 | m-D13 | H | Me | A-22 | Me | H |
| 11256 | 0 | m-D13 | H | Me | A-23 | Me | H |
| 11257 | 0 | m-D13 | H | Me | A-24 | Me | H |
| 11258 | 0 | m-D13 | H | Me | A-25 | Me | H |
| 11259 | 0 | m-D13 | H | Me | A-26 | Me | H |
| 11260 | 0 | m-D13 | H | Me | A-27 | Me | H |
| 11261 | 0 | m-D13 | H | Me | A-28 | Me | H |
| 11262 | 0 | m-D13 | H | Me | A-29 | Me | H |
| 11263 | 0 | m-D13 | H | Me | A-30 | Me | H |
| 11264 | 0 | m-D13 | H | Me | A-31 | Me | H |
| 11265 | 0 | m-D13 | H | Me | A-32 | Me | H |
| 11266 | 0 | m-D13 | H | Me | A-33 | Me | H |
| 11267 | 0 | m-D13 | H | Me | A-34 | Me | H |
| 11268 | 0 | m-D13 | H | Me | A-35 | Me | H |
| 11269 | 0 | m-D13 | H | Me | A-36 | Me | H |
| 11270 | 0 | m-D13 | H | Me | A-37 | Me | H |
| 11271 | 0 | m-D13 | H | Me | A-38 | Me | H |
| 11272 | 0 | m-D13 | H | Me | A-39 | Me | H |
| 11273 | 0 | m-D13 | H | Me | A-40 | Me | H |
| 11274 | 0 | m-D13 | H | Me | A-41 | Me | H |
| 11275 | 0 | m-D13 | H | Me | A-42 | Me | H |
| 11276 | 0 | m-D13 | H | Me | A-43 | Me | H |
| 11277 | 0 | m-D13 | H | Me | A-44 | Me | H |
| 11278 | 0 | m-D13 | H | Me | A-45 | Me | H |
| 11279 | 0 | m-D13 | H | Me | A-46 | Me | H |
| 11280 | 0 | m-D13 | H | Me | A-47 | Me | H |
| 11281 | 0 | m-D14 | H | Me | A-1 | Me | H |
| 11282 | 0 | m-D14 | H | Me | A-2 | Me | H |
| 11283 | 0 | m-D14 | H | Me | A-3 | Me | H |
| 11284 | 0 | m-D14 | H | Me | A-4 | Me | H |
| 11285 | 0 | m-D14 | H | Me | A-5 | Me | H |
| 11286 | 0 | m-D14 | H | Me | A-6 | Me | H |
| 11287 | 0 | m-D14 | H | Me | A-7 | Me | H |
| 11288 | 0 | m-D14 | H | Me | A-8 | Me | H |
| 11289 | 0 | m-D14 | H | Me | A-9 | Me | H |
| 11290 | 0 | m-D14 | H | Me | A-10 | Me | H |
| 11291 | 0 | m-D14 | H | Me | A-11 | Me | H |
| 11292 | 0 | m-D14 | H | Me | A-12 | Me | H |
| 11293 | 0 | m-D14 | H | Me | A-13 | Me | H |
| 11294 | 0 | m-D14 | H | Me | A-14 | Me | H |
| 11295 | 0 | m-D14 | H | Me | A-15 | Me | H |
| 11296 | 0 | m-D14 | H | Me | A-16 | Me | H |
| 11297 | 0 | m-D14 | H | Me | A-17 | Me | H |
| 11298 | 0 | m-D14 | H | Me | A-18 | Me | H |
| 11299 | 0 | m-D14 | H | Me | A-19 | Me | H |
| 11300 | 0 | m-D14 | H | Me | A-20 | Me | H |
| 11301 | 0 | m-D14 | H | Me | A-21 | Me | H |
| 11302 | 0 | m-D14 | H | Me | A-22 | Me | H |
| 11303 | 0 | m-D14 | H | Me | A-23 | Me | H |
| 11304 | 0 | m-D14 | H | Me | A-24 | Me | H |
| 11305 | 0 | m-D14 | H | Me | A-25 | Me | H |
| 11306 | 0 | m-D14 | H | Me | A-26 | Me | H |
| 11307 | 0 | m-D14 | H | Me | A-27 | Me | H |
| 11308 | 0 | m-D14 | H | Me | A-28 | Me | H |
| 11309 | 0 | m-D14 | H | Me | A-29 | Me | H |
| 11310 | 0 | m-D14 | H | Me | A-30 | Me | H |
| 11311 | 0 | m-D14 | H | Me | A-31 | Me | H |
| 11312 | 0 | m-D14 | H | Me | A-32 | Me | H |
| 11313 | 0 | m-D14 | H | Me | A-33 | Me | H |
| 11314 | 0 | m-D14 | H | Me | A-34 | Me | H |
| 11315 | 0 | m-D14 | H | Me | A-35 | Me | H |
| 11316 | 0 | m-D14 | H | Me | A-36 | Me | H |
| 11317 | 0 | m-D14 | H | Me | A-37 | Me | H |
| 11318 | 0 | m-D14 | H | Me | A-38 | Me | H |
| 11319 | 0 | m-D14 | H | Me | A-39 | Me | H |
| 11320 | 0 | m-D14 | H | Me | A-40 | Me | H |
| 11321 | 0 | m-D14 | H | Me | A-41 | Me | H |
| 11322 | 0 | m-D14 | H | Me | A-42 | Me | H |
| 11323 | 0 | m-D14 | H | Me | A-43 | Me | H |
| 11324 | 0 | m-D14 | H | Me | A-44 | Me | H |
| 11325 | 0 | m-D14 | H | Me | A-45 | Me | H |
| 11326 | 0 | m-D14 | H | Me | A-46 | Me | H |
| 11327 | 0 | m-D14 | H | Me | A-47 | Me | H |
| 11328 | 0 | m-D1 | tBu | H | A-1 | H | tBu |
| 11329 | 0 | m-D1 | tBu | H | A-2 | H | tBu |
| 11330 | 0 | m-D1 | tBu | H | A-3 | H | tBu |
| 11331 | 0 | m-D1 | tBu | H | A-4 | H | tBu |
| 11332 | 0 | m-D1 | tBu | H | A-5 | H | tBu |
| 11333 | 0 | m-D1 | tBu | H | A-6 | H | tBu |
| 11334 | 0 | m-D1 | tBu | H | A-7 | H | tBu |
| 11335 | 0 | m-D1 | tBu | H | A-8 | H | tBu |
| 11336 | 0 | m-D1 | tBu | H | A-9 | H | tBu |
| 11337 | 0 | m-D1 | tBu | H | A-10 | H | tBu |
| 11338 | 0 | m-D1 | tBu | H | A-11 | H | tBu |
| 11339 | 0 | m-D1 | tBu | H | A-12 | H | tBu |
| 11340 | 0 | m-D1 | tBu | H | A-13 | H | tBu |
| 11341 | 0 | m-D1 | tBu | H | A-14 | H | tBu |
| 11342 | 0 | m-D1 | tBu | H | A-15 | H | tBu |
| 11343 | 0 | m-D1 | tBu | H | A-16 | H | tBu |
| 11344 | 0 | m-D1 | tBu | H | A-17 | H | tBu |
| 11345 | 0 | m-D1 | tBu | H | A-18 | H | tBu |
| 11346 | 0 | m-D1 | tBu | H | A-19 | H | tBu |
| 11347 | 0 | m-D1 | tBu | H | A-20 | H | tBu |
| 11348 | 0 | m-D1 | tBu | H | A-21 | H | tBu |
| 11349 | 0 | m-D1 | tBu | H | A-22 | H | tBu |
| 11350 | 0 | m-D1 | tBu | H | A-23 | H | tBu |
| 11351 | 0 | m-D1 | tBu | H | A-24 | H | tBu |
| 11352 | 0 | m-D1 | tBu | H | A-25 | H | tBu |
| 11353 | 0 | m-D1 | tBu | H | A-26 | H | tBu |
| 11354 | 0 | m-D1 | tBu | H | A-27 | H | tBu |
| 11355 | 0 | m-D1 | tBu | H | A-28 | H | tBu |
| 11356 | 0 | m-D1 | tBu | H | A-29 | H | tBu |
| 11357 | 0 | m-D1 | tBu | H | A-30 | H | tBu |
| 11358 | 0 | m-D1 | tBu | H | A-31 | H | tBu |
| 11359 | 0 | m-D1 | tBu | H | A-32 | H | tBu |
| 11360 | 0 | m-D1 | tBu | H | A-33 | H | tBu |
| 11361 | 0 | m-D1 | tBu | H | A-34 | H | tBu |
| 11362 | 0 | m-D1 | tBu | H | A-35 | H | tBu |
| 11363 | 0 | m-D1 | tBu | H | A-36 | H | tBu |
| 11364 | 0 | m-D1 | tBu | H | A-37 | H | tBu |
| 11365 | 0 | m-D1 | tBu | H | A-38 | H | tBu |
| 11366 | 0 | m-D1 | tBu | H | A-39 | H | tBu |
| 11367 | 0 | m-D1 | tBu | H | A-40 | H | tBu |
| 11368 | 0 | m-D1 | tBu | H | A-41 | H | tBu |
| 11369 | 0 | m-D1 | tBu | H | A-42 | H | tBu |
| 11370 | 0 | m-D1 | tBu | H | A-43 | H | tBu |
| 11371 | 0 | m-D1 | tBu | H | A-44 | H | tBu |
| 11372 | 0 | m-D1 | tBu | H | A-45 | H | tBu |
| 11373 | 0 | m-D1 | tBu | H | A-46 | H | tBu |
| 11374 | 0 | m-D1 | tBu | H | A-47 | H | tBu |
| 11375 | 0 | m-D2 | tBu | H | A-1 | H | tBu |
| 11376 | 0 | m-D2 | tBu | H | A-2 | H | tBu |
| 11377 | 0 | m-D2 | tBu | H | A-3 | H | tBu |
| 11378 | 0 | m-D2 | tBu | H | A-4 | H | tBu |
| 11379 | 0 | m-D2 | tBu | H | A-5 | H | tBu |
| 11380 | 0 | m-D2 | tBu | H | A-6 | H | tBu |
| 11381 | 0 | m-D2 | tBu | H | A-7 | H | tBu |
| 11382 | 0 | m-D2 | tBu | H | A-8 | H | tBu |
| 11383 | 0 | m-D2 | tBu | H | A-9 | H | tBu |
| 11384 | 0 | m-D2 | tBu | H | A-10 | H | tBu |
| 11385 | 0 | m-D2 | tBu | H | A-11 | H | tBu |
| 11386 | 0 | m-D2 | tBu | H | A-12 | H | tBu |
| 11387 | 0 | m-D2 | tBu | H | A-13 | H | tBu |
| 11388 | 0 | m-D2 | tBu | H | A-14 | H | tBu |
| 11389 | 0 | m-D2 | tBu | H | A-15 | H | tBu |
| 11390 | 0 | m-D2 | tBu | H | A-16 | H | tBu |
| 11391 | 0 | m-D2 | tBu | H | A-17 | H | tBu |
| 11392 | 0 | m-D2 | tBu | H | A-18 | H | tBu |
| 11393 | 0 | m-D2 | tBu | H | A-19 | H | tBu |
| 11394 | 0 | m-D2 | tBu | H | A-20 | H | tBu |
| 11395 | 0 | m-D2 | tBu | H | A-21 | H | tBu |
| 11396 | 0 | m-D2 | tBu | H | A-22 | H | tBu |
| 11397 | 0 | m-D2 | tBu | H | A-23 | H | tBu |
| 11398 | 0 | m-D2 | tBu | H | A-24 | H | tBu |
| 11399 | 0 | m-D2 | tBu | H | A-25 | H | tBu |
| 11400 | 0 | m-D2 | tBu | H | A-26 | H | tBu |
| 11401 | 0 | m-D2 | tBu | H | A-27 | H | tBu |
| 11402 | 0 | m-D2 | tBu | H | A-28 | H | tBu |
| 11403 | 0 | m-D2 | tBu | H | A-29 | H | tBu |
| 11404 | 0 | m-D2 | tBu | H | A-30 | H | tBu |
| 11405 | 0 | m-D2 | tBu | H | A-31 | H | tBu |
| 11406 | 0 | m-D2 | tBu | H | A-32 | H | tBu |
| 11407 | 0 | m-D2 | tBu | H | A-33 | H | tBu |
| 11408 | 0 | m-D2 | tBu | H | A-34 | H | tBu |
| 11409 | 0 | m-D2 | tBu | H | A-35 | H | tBu |
| 11410 | 0 | m-D2 | tBu | H | A-36 | H | tBu |
| 11411 | 0 | m-D2 | tBu | H | A-37 | H | tBu |
| 11412 | 0 | m-D2 | tBu | H | A-38 | H | tBu |
| 11413 | 0 | m-D2 | tBu | H | A-39 | H | tBu |
| 11414 | 0 | m-D2 | tBu | H | A-40 | H | tBu |
| 11415 | 0 | m-D2 | tBu | H | A-41 | H | tBu |
| 11416 | 0 | m-D2 | tBu | H | A-42 | H | tBu |
| 11417 | 0 | m-D2 | tBu | H | A-43 | H | tBu |
| 11418 | 0 | m-D2 | tBu | H | A-44 | H | tBu |
| 11419 | 0 | m-D2 | tBu | H | A-45 | H | tBu |
| 11420 | 0 | m-D2 | tBu | H | A-46 | H | tBu |
| 11421 | 0 | m-D2 | tBu | H | A-47 | H | tBu |
| 11422 | 0 | m-D3 | tBu | H | A-1 | H | tBu |
| 11423 | 0 | m-D3 | tBu | H | A-2 | H | tBu |
| 11424 | 0 | m-D3 | tBu | H | A-3 | H | tBu |
| 11425 | 0 | m-D3 | tBu | H | A-4 | H | tBu |
| 11426 | 0 | m-D3 | tBu | H | A-5 | H | tBu |
| 11427 | 0 | m-D3 | tBu | H | A-6 | H | tBu |
| 11428 | 0 | m-D3 | tBu | H | A-7 | H | tBu |
| 11429 | 0 | m-D3 | tBu | H | A-8 | H | tBu |
| 11430 | 0 | m-D3 | tBu | H | A-9 | H | tBu |
| 11431 | 0 | m-D3 | tBu | H | A-10 | H | tBu |
| 11432 | 0 | m-D3 | tBu | H | A-11 | H | tBu |
| 11433 | 0 | m-D3 | tBu | H | A-12 | H | tBu |
| 11434 | 0 | m-D3 | tBu | H | A-13 | H | tBu |
| 11435 | 0 | m-D3 | tBu | H | A-14 | H | tBu |
| 11436 | 0 | m-D3 | tBu | H | A-15 | H | tBu |
| 11437 | 0 | m-D3 | tBu | H | A-16 | H | tBu |
| 11438 | 0 | m-D3 | tBu | H | A-17 | H | tBu |
| 11439 | 0 | m-D3 | tBu | H | A-18 | H | tBu |
| 11440 | 0 | m-D3 | tBu | H | A-19 | H | tBu |
| 11441 | 0 | m-D3 | tBu | H | A-20 | H | tBu |
| 11442 | 0 | m-D3 | tBu | H | A-21 | H | tBu |
| 11443 | 0 | m-D3 | tBu | H | A-22 | H | tBu |
| 11444 | 0 | m-D3 | tBu | H | A-23 | H | tBu |
| 11445 | 0 | m-D3 | tBu | H | A-24 | H | tBu |
| 11446 | 0 | m-D3 | tBu | H | A-25 | H | tBu |
| 11447 | 0 | m-D3 | tBu | H | A-26 | H | tBu |
| 11448 | 0 | m-D3 | tBu | H | A-27 | H | tBu |
| 11449 | 0 | m-D3 | tBu | H | A-28 | H | tBu |
| 11450 | 0 | m-D3 | tBu | H | A-29 | H | tBu |
| 11451 | 0 | m-D3 | tBu | H | A-30 | H | tBu |
| 11452 | 0 | m-D3 | tBu | H | A-31 | H | tBu |
| 11453 | 0 | m-D3 | tBu | H | A-32 | H | tBu |
| 11454 | 0 | m-D3 | tBu | H | A-33 | H | tBu |
| 11455 | 0 | m-D3 | tBu | H | A-34 | H | tBu |
| 11456 | 0 | m-D3 | tBu | H | A-35 | H | tBu |
| 11457 | 0 | m-D3 | tBu | H | A-36 | H | tBu |
| 11458 | 0 | m-D3 | tBu | H | A-37 | H | tBu |
| 11459 | 0 | m-D3 | tBu | H | A-38 | H | tBu |
| 11460 | 0 | m-D3 | tBu | H | A-39 | H | tBu |
| 11461 | 0 | m-D3 | tBu | H | A-40 | H | tBu |
| 11462 | 0 | m-D3 | tBu | H | A-41 | H | tBu |
| 11463 | 0 | m-D3 | tBu | H | A-42 | H | tBu |
| 11464 | 0 | m-D3 | tBu | H | A-43 | H | tBu |
| 11465 | 0 | m-D3 | tBu | H | A-44 | H | tBu |
| 11466 | 0 | m-D3 | tBu | H | A-45 | H | tBu |
| 11467 | 0 | m-D3 | tBu | H | A-46 | H | tBu |
| 11468 | 0 | m-D3 | tBu | H | A-47 | H | tBu |
| 11469 | 0 | m-D4 | tBu | H | A-1 | H | tBu |
| 11470 | 0 | m-D4 | tBu | H | A-2 | H | tBu |
| 11471 | 0 | m-D4 | tBu | H | A-3 | H | tBu |
| 11472 | 0 | m-D4 | tBu | H | A-4 | H | tBu |
| 11473 | 0 | m-D4 | tBu | H | A-5 | H | tBu |
| 11474 | 0 | m-D4 | tBu | H | A-6 | H | tBu |
| 11475 | 0 | m-D4 | tBu | H | A-7 | H | tBu |
| 11476 | 0 | m-D4 | tBu | H | A-8 | H | tBu |
| 11477 | 0 | m-D4 | tBu | H | A-9 | H | tBu |
| 11478 | 0 | m-D4 | tBu | H | A-10 | H | tBu |
| 11479 | 0 | m-D4 | tBu | H | A-11 | H | tBu |
| 11480 | 0 | m-D4 | tBu | H | A-12 | H | tBu |
| 11481 | 0 | m-D4 | tBu | H | A-13 | H | tBu |
| 11482 | 0 | m-D4 | tBu | H | A-14 | H | tBu |
| 11483 | 0 | m-D4 | tBu | H | A-15 | H | tBu |
| 11484 | 0 | m-D4 | tBu | H | A-16 | H | tBu |
| 11485 | 0 | m-D4 | tBu | H | A-17 | H | tBu |
| 11486 | 0 | m-D4 | tBu | H | A-18 | H | tBu |
| 11487 | 0 | m-D4 | tBu | H | A-19 | H | tBu |
| 11488 | 0 | m-D4 | tBu | H | A-20 | H | tBu |
| 11489 | 0 | m-D4 | tBu | H | A-21 | H | tBu |
| 11490 | 0 | m-D4 | tBu | H | A-22 | H | tBu |
| 11491 | 0 | m-D4 | tBu | H | A-23 | H | tBu |
| 11492 | 0 | m-D4 | tBu | H | A-24 | H | tBu |
| 11493 | 0 | m-D4 | tBu | H | A-25 | H | tBu |
| 11494 | 0 | m-D4 | tBu | H | A-26 | H | tBu |
| 11495 | 0 | m-D4 | tBu | H | A-27 | H | tBu |
| 11496 | 0 | m-D4 | tBu | H | A-28 | H | tBu |
| 11497 | 0 | m-D4 | tBu | H | A-29 | H | tBu |
| 11498 | 0 | m-D4 | tBu | H | A-30 | H | tBu |
| 11499 | 0 | m-D4 | tBu | H | A-31 | H | tBu |
| 11500 | 0 | m-D4 | tBu | H | A-32 | H | tBu |
| 11501 | 0 | m-D4 | tBu | H | A-33 | H | tBu |
| 11502 | 0 | m-D4 | tBu | H | A-34 | H | tBu |
| 11503 | 0 | m-D4 | tBu | H | A-35 | H | tBu |
| 11504 | 0 | m-D4 | tBu | H | A-36 | H | tBu |
| 11505 | 0 | m-D4 | tBu | H | A-37 | H | tBu |
| 11506 | 0 | m-D4 | tBu | H | A-38 | H | tBu |
| 11507 | 0 | m-D4 | tBu | H | A-39 | H | tBu |
| 11508 | 0 | m-D4 | tBu | H | A-40 | H | tBu |
| 11509 | 0 | m-D4 | tBu | H | A-41 | H | tBu |
| 11510 | 0 | m-D4 | tBu | H | A-42 | H | tBu |
| 11511 | 0 | m-D4 | tBu | H | A-43 | H | tBu |
| 11512 | 0 | m-D4 | tBu | H | A-44 | H | tBu |
| 11513 | 0 | m-D4 | tBu | H | A-45 | H | tBu |
| 11514 | 0 | m-D4 | tBu | H | A-46 | H | tBu |
| 11515 | 0 | m-D4 | tBu | H | A-47 | H | tBu |
| 11516 | 0 | m-D5 | tBu | H | A-1 | H | tBu |
| 11517 | 0 | m-D5 | tBu | H | A-2 | H | tBu |
| 11518 | 0 | m-D5 | tBu | H | A-3 | H | tBu |
| 11519 | 0 | m-D5 | tBu | H | A-4 | H | tBu |
| 11520 | 0 | m-D5 | tBu | H | A-5 | H | tBu |
| 11521 | 0 | m-D5 | tBu | H | A-6 | H | tBu |
| 11522 | 0 | m-D5 | tBu | H | A-7 | H | tBu |
| 11523 | 0 | m-D5 | tBu | H | A-8 | H | tBu |
| 11524 | 0 | m-D5 | tBu | H | A-9 | H | tBu |
| 11525 | 0 | m-D5 | tBu | H | A-10 | H | tBu |
| 11526 | 0 | m-D5 | tBu | H | A-11 | H | tBu |
| 11527 | 0 | m-D5 | tBu | H | A-12 | H | tBu |
| 11528 | 0 | m-D5 | tBu | H | A-13 | H | tBu |
| 11529 | 0 | m-D5 | tBu | H | A-14 | H | tBu |
| 11530 | 0 | m-D5 | tBu | H | A-15 | H | tBu |
| 11531 | 0 | m-D5 | tBu | H | A-16 | H | tBu |
| 11532 | 0 | m-D5 | tBu | H | A-17 | H | tBu |
| 11533 | 0 | m-D5 | tBu | H | A-18 | H | tBu |
| 11534 | 0 | m-D5 | tBu | H | A-19 | H | tBu |
| 11535 | 0 | m-D5 | tBu | H | A-20 | H | tBu |
| 11536 | 0 | m-D5 | tBu | H | A-21 | H | tBu |
| 11537 | 0 | m-D5 | tBu | H | A-22 | H | tBu |
| 11538 | 0 | m-D5 | tBu | H | A-23 | H | tBu |
| 11539 | 0 | m-D5 | tBu | H | A-24 | H | tBu |
| 11540 | 0 | m-D5 | tBu | H | A-25 | H | tBu |
| 11541 | 0 | m-D5 | tBu | H | A-26 | H | tBu |
| 11542 | 0 | m-D5 | tBu | H | A-27 | H | tBu |
| 11543 | 0 | m-D5 | tBu | H | A-28 | H | tBu |
| 11544 | 0 | m-D5 | tBu | H | A-29 | H | tBu |
| 11545 | 0 | m-D5 | tBu | H | A-30 | H | tBu |
| 11546 | 0 | m-D5 | tBu | H | A-31 | H | tBu |
| 11547 | 0 | m-D5 | tBu | H | A-32 | H | tBu |
| 11548 | 0 | m-D5 | tBu | H | A-33 | H | tBu |
| 11549 | 0 | m-D5 | tBu | H | A-34 | H | tBu |
| 11550 | 0 | m-D5 | tBu | H | A-35 | H | tBu |
| 11551 | 0 | m-D5 | tBu | H | A-36 | H | tBu |
| 11552 | 0 | m-D5 | tBu | H | A-37 | H | tBu |
| 11553 | 0 | m-D5 | tBu | H | A-38 | H | tBu |
| 11554 | 0 | m-D5 | tBu | H | A-39 | H | tBu |
| 11555 | 0 | m-D5 | tBu | H | A-40 | H | tBu |
| 11556 | 0 | m-D5 | tBu | H | A-41 | H | tBu |
| 11557 | 0 | m-D5 | tBu | H | A-42 | H | tBu |
| 11558 | 0 | m-D5 | tBu | H | A-43 | H | tBu |
| 11559 | 0 | m-D5 | tBu | H | A-44 | H | tBu |
| 11560 | 0 | m-D5 | tBu | H | A-45 | H | tBu |
| 11561 | 0 | m-D5 | tBu | H | A-46 | H | tBu |
| 11562 | 0 | m-D5 | tBu | H | A-47 | H | tBu |
| 11563 | 0 | m-D6 | tBu | H | A-1 | H | tBu |
| 11564 | 0 | m-D6 | tBu | H | A-2 | H | tBu |
| 11565 | 0 | m-D6 | tBu | H | A-3 | H | tBu |
| 11566 | 0 | m-D6 | tBu | H | A-4 | H | tBu |
| 11567 | 0 | m-D6 | tBu | H | A-5 | H | tBu |
| 11568 | 0 | m-D6 | tBu | H | A-6 | H | tBu |
| 11569 | 0 | m-D6 | tBu | H | A-7 | H | tBu |
| 11570 | 0 | m-D6 | tBu | H | A-8 | H | tBu |
| 11571 | 0 | m-D6 | tBu | H | A-9 | H | tBu |
| 11572 | 0 | m-D6 | tBu | H | A-10 | H | tBu |
| 11573 | 0 | m-D6 | tBu | H | A-11 | H | tBu |
| 11574 | 0 | m-D6 | tBu | H | A-12 | H | tBu |
| 11575 | 0 | m-D6 | tBu | H | A-13 | H | tBu |
| 11576 | 0 | m-D6 | tBu | H | A-14 | H | tBu |
| 11577 | 0 | m-D6 | tBu | H | A-15 | H | tBu |
| 11578 | 0 | m-D6 | tBu | H | A-16 | H | tBu |
| 11579 | 0 | m-D6 | tBu | H | A-17 | H | tBu |
| 11580 | 0 | m-D6 | tBu | H | A-18 | H | tBu |
| 11581 | 0 | m-D6 | tBu | H | A-19 | H | tBu |
| 11582 | 0 | m-D6 | tBu | H | A-20 | H | tBu |
| 11583 | 0 | m-D6 | tBu | H | A-21 | H | tBu |
| 11584 | 0 | m-D6 | tBu | H | A-22 | H | tBu |
| 11585 | 0 | m-D6 | tBu | H | A-23 | H | tBu |
| 11586 | 0 | m-D6 | tBu | H | A-24 | H | tBu |
| 11587 | 0 | m-D6 | tBu | H | A-25 | H | tBu |
| 11588 | 0 | m-D6 | tBu | H | A-26 | H | tBu |
| 11589 | 0 | m-D6 | tBu | H | A-27 | H | tBu |
| 11590 | 0 | m-D6 | tBu | H | A-28 | H | tBu |
| 11591 | 0 | m-D6 | tBu | H | A-29 | H | tBu |
| 11592 | 0 | m-D6 | tBu | H | A-30 | H | tBu |
| 11593 | 0 | m-D6 | tBu | H | A-31 | H | tBu |
| 11594 | 0 | m-D6 | tBu | H | A-32 | H | tBu |
| 11595 | 0 | m-D6 | tBu | H | A-33 | H | tBu |
| 11596 | 0 | m-D6 | tBu | H | A-34 | H | tBu |
| 11597 | 0 | m-D6 | tBu | H | A-35 | H | tBu |
| 11598 | 0 | m-D6 | tBu | H | A-36 | H | tBu |
| 11599 | 0 | m-D6 | tBu | H | A-37 | H | tBu |
| 11600 | 0 | m-D6 | tBu | H | A-38 | H | tBu |
| 11601 | 0 | m-D6 | tBu | H | A-39 | H | tBu |
| 11602 | 0 | m-D6 | tBu | H | A-40 | H | tBu |
| 11603 | 0 | m-D6 | tBu | H | A-41 | H | tBu |
| 11604 | 0 | m-D6 | tBu | H | A-42 | H | tBu |
| 11605 | 0 | m-D6 | tBu | H | A-43 | H | tBu |
| 11606 | 0 | m-D6 | tBu | H | A-44 | H | tBu |
| 11607 | 0 | m-D6 | tBu | H | A-45 | H | tBu |
| 11608 | 0 | m-D6 | tBu | H | A-46 | H | tBu |
| 11609 | 0 | m-D6 | tBu | H | A-47 | H | tBu |
| 11610 | 0 | m-D7 | tBu | H | A-1 | H | tBu |
| 11611 | 0 | m-D7 | tBu | H | A-2 | H | tBu |
| 11612 | 0 | m-D7 | tBu | H | A-3 | H | tBu |
| 11613 | 0 | m-D7 | tBu | H | A-4 | H | tBu |
| 11614 | 0 | m-D7 | tBu | H | A-5 | H | tBu |
| 11615 | 0 | m-D7 | tBu | H | A-6 | H | tBu |
| 11616 | 0 | m-D7 | tBu | H | A-7 | H | tBu |
| 11617 | 0 | m-D7 | tBu | H | A-8 | H | tBu |
| 11618 | 0 | m-D7 | tBu | H | A-9 | H | tBu |
| 11619 | 0 | m-D7 | tBu | H | A-10 | H | tBu |
| 11620 | 0 | m-D7 | tBu | H | A-11 | H | tBu |
| 11621 | 0 | m-D7 | tBu | H | A-12 | H | tBu |
| 11622 | 0 | m-D7 | tBu | H | A-13 | H | tBu |
| 11623 | 0 | m-D7 | tBu | H | A-14 | H | tBu |
| 11624 | 0 | m-D7 | tBu | H | A-15 | H | tBu |
| 11625 | 0 | m-D7 | tBu | H | A-16 | H | tBu |
| 11626 | 0 | m-D7 | tBu | H | A-17 | H | tBu |
| 11627 | 0 | m-D7 | tBu | H | A-18 | H | tBu |
| 11628 | 0 | m-D7 | tBu | H | A-19 | H | tBu |
| 11629 | 0 | m-D7 | tBu | H | A-20 | H | tBu |
| 11630 | 0 | m-D7 | tBu | H | A-21 | H | tBu |
| 11631 | 0 | m-D7 | tBu | H | A-22 | H | tBu |
| 11632 | 0 | m-D7 | tBu | H | A-23 | H | tBu |
| 11633 | 0 | m-D7 | tBu | H | A-24 | H | tBu |
| 11634 | 0 | m-D7 | tBu | H | A-25 | H | tBu |
| 11635 | 0 | m-D7 | tBu | H | A-26 | H | tBu |
| 11636 | 0 | m-D7 | tBu | H | A-27 | H | tBu |
| 11637 | 0 | m-D7 | tBu | H | A-28 | H | tBu |
| 11638 | 0 | m-D7 | tBu | H | A-29 | H | tBu |
| 11639 | 0 | m-D7 | tBu | H | A-30 | H | tBu |
| 11640 | 0 | m-D7 | tBu | H | A-31 | H | tBu |
| 11641 | 0 | m-D7 | tBu | H | A-32 | H | tBu |
| 11642 | 0 | m-D7 | tBu | H | A-33 | H | tBu |
| 11643 | 0 | m-D7 | tBu | H | A-34 | H | tBu |
| 11644 | 0 | m-D7 | tBu | H | A-35 | H | tBu |
| 11645 | 0 | m-D7 | tBu | H | A-36 | H | tBu |
| 11646 | 0 | m-D7 | tBu | H | A-37 | H | tBu |
| 11647 | 0 | m-D7 | tBu | H | A-38 | H | tBu |
| 11648 | 0 | m-D7 | tBu | H | A-39 | H | tBu |
| 11649 | 0 | m-D7 | tBu | H | A-40 | H | tBu |
| 11650 | 0 | m-D7 | tBu | H | A-41 | H | tBu |
| 11651 | 0 | m-D7 | tBu | H | A-42 | H | tBu |
| 11652 | 0 | m-D7 | tBu | H | A-43 | H | tBu |
| 11653 | 0 | m-D7 | tBu | H | A-44 | H | tBu |
| 11654 | 0 | m-D7 | tBu | H | A-45 | H | tBu |
| 11655 | 0 | m-D7 | tBu | H | A-46 | H | tBu |
| 11656 | 0 | m-D7 | tBu | H | A-47 | H | tBu |
| 11657 | 0 | m-D8 | tBu | H | A-1 | H | tBu |
| 11658 | 0 | m-D8 | tBu | H | A-2 | H | tBu |
| 11659 | 0 | m-D8 | tBu | H | A-3 | H | tBu |
| 11660 | 0 | m-D8 | tBu | H | A-4 | H | tBu |
| 11661 | 0 | m-D8 | tBu | H | A-5 | H | tBu |
| 11662 | 0 | m-D8 | tBu | H | A-6 | H | tBu |
| 11663 | 0 | m-D8 | tBu | H | A-7 | H | tBu |
| 11664 | 0 | m-D8 | tBu | H | A-8 | H | tBu |
| 11665 | 0 | m-D8 | tBu | H | A-9 | H | tBu |
| 11666 | 0 | m-D8 | tBu | H | A-10 | H | tBu |
| 11667 | 0 | m-D8 | tBu | H | A-11 | H | tBu |
| 11668 | 0 | m-D8 | tBu | H | A-12 | H | tBu |
| 11669 | 0 | m-D8 | tBu | H | A-13 | H | tBu |
| 11670 | 0 | m-D8 | tBu | H | A-14 | H | tBu |
| 11671 | 0 | m-D8 | tBu | H | A-15 | H | tBu |
| 11672 | 0 | m-D8 | tBu | H | A-16 | H | tBu |
| 11673 | 0 | m-D8 | tBu | H | A-17 | H | tBu |
| 11674 | 0 | m-D8 | tBu | H | A-18 | H | tBu |
| 11675 | 0 | m-D8 | tBu | H | A-19 | H | tBu |
| 11676 | 0 | m-D8 | tBu | H | A-20 | H | tBu |
| 11677 | 0 | m-D8 | tBu | H | A-21 | H | tBu |
| 11678 | 0 | m-D8 | tBu | H | A-22 | H | tBu |
| 11679 | 0 | m-D8 | tBu | H | A-23 | H | tBu |
| 11680 | 0 | m-D8 | tBu | H | A-24 | H | tBu |
| 11681 | 0 | m-D8 | tBu | H | A-25 | H | tBu |
| 11682 | 0 | m-D8 | tBu | H | A-26 | H | tBu |
| 11683 | 0 | m-D8 | tBu | H | A-27 | H | tBu |
| 11684 | 0 | m-D8 | tBu | H | A-28 | H | tBu |
| 11685 | 0 | m-D8 | tBu | H | A-29 | H | tBu |
| 11686 | 0 | m-D8 | tBu | H | A-30 | H | tBu |
| 11687 | 0 | m-D8 | tBu | H | A-31 | H | tBu |
| 11688 | 0 | m-D8 | tBu | H | A-32 | H | tBu |
| 11689 | 0 | m-D8 | tBu | H | A-33 | H | tBu |
| 11690 | 0 | m-D8 | tBu | H | A-34 | H | tBu |
| 11691 | 0 | m-D8 | tBu | H | A-35 | H | tBu |
| 11692 | 0 | m-D8 | tBu | H | A-36 | H | tBu |
| 11693 | 0 | m-D8 | tBu | H | A-37 | H | tBu |
| 11694 | 0 | m-D8 | tBu | H | A-38 | H | tBu |
| 11695 | 0 | m-D8 | tBu | H | A-39 | H | tBu |
| 11696 | 0 | m-D8 | tBu | H | A-40 | H | tBu |
| 11697 | 0 | m-D8 | tBu | H | A-41 | H | tBu |
| 11698 | 0 | m-D8 | tBu | H | A-42 | H | tBu |
| 11699 | 0 | m-D8 | tBu | H | A-43 | H | tBu |
| 11700 | 0 | m-D8 | tBu | H | A-44 | H | tBu |
| 11701 | 0 | m-D8 | tBu | H | A-45 | H | tBu |
| 11702 | 0 | m-D8 | tBu | H | A-46 | H | tBu |
| 11703 | 0 | m-D8 | tBu | H | A-47 | H | tBu |
| 11704 | 0 | m-D9 | tBu | H | A-1 | H | tBu |
| 11705 | 0 | m-D9 | tBu | H | A-2 | H | tBu |
| 11706 | 0 | m-D9 | tBu | H | A-3 | H | tBu |
| 11707 | 0 | m-D9 | tBu | H | A-4 | H | tBu |
| 11708 | 0 | m-D9 | tBu | H | A-5 | H | tBu |
| 11709 | 0 | m-D9 | tBu | H | A-6 | H | tBu |
| 11710 | 0 | m-D9 | tBu | H | A-7 | H | tBu |
| 11711 | 0 | m-D9 | tBu | H | A-8 | H | tBu |
| 11712 | 0 | m-D9 | tBu | H | A-9 | H | tBu |
| 11713 | 0 | m-D9 | tBu | H | A-10 | H | tBu |
| 11714 | 0 | m-D9 | tBu | H | A-11 | H | tBu |
| 11715 | 0 | m-D9 | tBu | H | A-12 | H | tBu |
| 11716 | 0 | m-D9 | tBu | H | A-13 | H | tBu |
| 11717 | 0 | m-D9 | tBu | H | A-14 | H | tBu |
| 11718 | 0 | m-D9 | tBu | H | A-15 | H | tBu |
| 11719 | 0 | m-D9 | tBu | H | A-16 | H | tBu |
| 11720 | 0 | m-D9 | tBu | H | A-17 | H | tBu |
| 11721 | 0 | m-D9 | tBu | H | A-18 | H | tBu |
| 11722 | 0 | m-D9 | tBu | H | A-19 | H | tBu |
| 11723 | 0 | m-D9 | tBu | H | A-20 | H | tBu |
| 11724 | 0 | m-D9 | tBu | H | A-21 | H | tBu |
| 11725 | 0 | m-D9 | tBu | H | A-22 | H | tBu |
| 11726 | 0 | m-D9 | tBu | H | A-23 | H | tBu |
| 11727 | 0 | m-D9 | tBu | H | A-24 | H | tBu |
| 11728 | 0 | m-D9 | tBu | H | A-25 | H | tBu |
| 11729 | 0 | m-D9 | tBu | H | A-26 | H | tBu |
| 11730 | 0 | m-D9 | tBu | H | A-27 | H | tBu |
| 11731 | 0 | m-D9 | tBu | H | A-28 | H | tBu |
| 11732 | 0 | m-D9 | tBu | H | A-29 | H | tBu |
| 11733 | 0 | m-D9 | tBu | H | A-30 | H | tBu |
| 11734 | 0 | m-D9 | tBu | H | A-31 | H | tBu |
| 11735 | 0 | m-D9 | tBu | H | A-32 | H | tBu |
| 11736 | 0 | m-D9 | tBu | H | A-33 | H | tBu |
| 11737 | 0 | m-D9 | tBu | H | A-34 | H | tBu |
| 11738 | 0 | m-D9 | tBu | H | A-35 | H | tBu |
| 11739 | 0 | m-D9 | tBu | H | A-36 | H | tBu |
| 11740 | 0 | m-D9 | tBu | H | A-37 | H | tBu |
| 11741 | 0 | m-D9 | tBu | H | A-38 | H | tBu |
| 11742 | 0 | m-D9 | tBu | H | A-39 | H | tBu |
| 11743 | 0 | m-D9 | tBu | H | A-40 | H | tBu |
| 11744 | 0 | m-D9 | tBu | H | A-41 | H | tBu |
| 11745 | 0 | m-D9 | tBu | H | A-42 | H | tBu |
| 11746 | 0 | m-D9 | tBu | H | A-43 | H | tBu |
| 11747 | 0 | m-D9 | tBu | H | A-44 | H | tBu |
| 11748 | 0 | m-D9 | tBu | H | A-45 | H | tBu |
| 11749 | 0 | m-D9 | tBu | H | A-46 | H | tBu |
| 11750 | 0 | m-D9 | tBu | H | A-47 | H | tBu |
| 11751 | 0 | m-D10 | tBu | H | A-1 | H | tBu |
| 11752 | 0 | m-D10 | tBu | H | A-2 | H | tBu |
| 11753 | 0 | m-D10 | tBu | H | A-3 | H | tBu |
| 11754 | 0 | m-D10 | tBu | H | A-4 | H | tBu |
| 11755 | 0 | m-D10 | tBu | H | A-5 | H | tBu |
| 11756 | 0 | m-D10 | tBu | H | A-6 | H | tBu |
| 11757 | 0 | m-D10 | tBu | H | A-7 | H | tBu |
| 11758 | 0 | m-D10 | tBu | H | A-8 | H | tBu |
| 11759 | 0 | m-D10 | tBu | H | A-9 | H | tBu |
| 11760 | 0 | m-D10 | tBu | H | A-10 | H | tBu |
| 11761 | 0 | m-D10 | tBu | H | A-11 | H | tBu |
| 11762 | 0 | m-D10 | tBu | H | A-12 | H | tBu |
| 11763 | 0 | m-D10 | tBu | H | A-13 | H | tBu |
| 11764 | 0 | m-D10 | tBu | H | A-14 | H | tBu |
| 11765 | 0 | m-D10 | tBu | H | A-15 | H | tBu |
| 11766 | 0 | m-D10 | tBu | H | A-16 | H | tBu |
| 11767 | 0 | m-D10 | tBu | H | A-17 | H | tBu |
| 11768 | 0 | m-D10 | tBu | H | A-18 | H | tBu |
| 11769 | 0 | m-D10 | tBu | H | A-19 | H | tBu |
| 11770 | 0 | m-D10 | tBu | H | A-20 | H | tBu |
| 11771 | 0 | m-D10 | tBu | H | A-21 | H | tBu |
| 11772 | 0 | m-D10 | tBu | H | A-22 | H | tBu |
| 11773 | 0 | m-D10 | tBu | H | A-23 | H | tBu |
| 11774 | 0 | m-D10 | tBu | H | A-24 | H | tBu |
| 11775 | 0 | m-D10 | tBu | H | A-25 | H | tBu |
| 11776 | 0 | m-D10 | tBu | H | A-26 | H | tBu |
| 11777 | 0 | m-D10 | tBu | H | A-27 | H | tBu |
| 11778 | 0 | m-D10 | tBu | H | A-28 | H | tBu |
| 11779 | 0 | m-D10 | tBu | H | A-29 | H | tBu |
| 11780 | 0 | m-D10 | tBu | H | A-30 | H | tBu |
| 11781 | 0 | m-D10 | tBu | H | A-31 | H | tBu |
| 11782 | 0 | m-D10 | tBu | H | A-32 | H | tBu |
| 11783 | 0 | m-D10 | tBu | H | A-33 | H | tBu |
| 11784 | 0 | m-D10 | tBu | H | A-34 | H | tBu |
| 11785 | 0 | m-D10 | tBu | H | A-35 | H | tBu |
| 11786 | 0 | m-D10 | tBu | H | A-36 | H | tBu |
| 11787 | 0 | m-D10 | tBu | H | A-37 | H | tBu |
| 11788 | 0 | m-D10 | tBu | H | A-38 | H | tBu |
| 11789 | 0 | m-D10 | tBu | H | A-39 | H | tBu |
| 11790 | 0 | m-D10 | tBu | H | A-40 | H | tBu |
| 11791 | 0 | m-D10 | tBu | H | A-41 | H | tBu |
| 11792 | 0 | m-D10 | tBu | H | A-42 | H | tBu |
| 11793 | 0 | m-D10 | tBu | H | A-43 | H | tBu |
| 11794 | 0 | m-D10 | tBu | H | A-44 | H | tBu |
| 11795 | 0 | m-D10 | tBu | H | A-45 | H | tBu |
| 11796 | 0 | m-D10 | tBu | H | A-46 | H | tBu |
| 11797 | 0 | m-D10 | tBu | H | A-47 | H | tBu |
| 11798 | 0 | m-D11 | tBu | H | A-1 | H | tBu |
| 11799 | 0 | m-D11 | tBu | H | A-2 | H | tBu |
| 11800 | 0 | m-D11 | tBu | H | A-3 | H | tBu |
| 11801 | 0 | m-D11 | tBu | H | A-4 | H | tBu |
| 11802 | 0 | m-D11 | tBu | H | A-5 | H | tBu |
| 11803 | 0 | m-D11 | tBu | H | A-6 | H | tBu |
| 11804 | 0 | m-D11 | tBu | H | A-7 | H | tBu |
| 11805 | 0 | m-D11 | tBu | H | A-8 | H | tBu |
| 11806 | 0 | m-D11 | tBu | H | A-9 | H | tBu |
| 11807 | 0 | m-D11 | tBu | H | A-10 | H | tBu |
| 11808 | 0 | m-D11 | tBu | H | A-11 | H | tBu |
| 11809 | 0 | m-D11 | tBu | H | A-12 | H | tBu |
| 11810 | 0 | m-D11 | tBu | H | A-13 | H | tBu |
| 11811 | 0 | m-D11 | tBu | H | A-14 | H | tBu |
| 11812 | 0 | m-D11 | tBu | H | A-15 | H | tBu |
| 11813 | 0 | m-D11 | tBu | H | A-16 | H | tBu |
| 11814 | 0 | m-D11 | tBu | H | A-17 | H | tBu |
| 11815 | 0 | m-D11 | tBu | H | A-18 | H | tBu |
| 11816 | 0 | m-D11 | tBu | H | A-19 | H | tBu |
| 11817 | 0 | m-D11 | tBu | H | A-20 | H | tBu |
| 11818 | 0 | m-D11 | tBu | H | A-21 | H | tBu |
| 11819 | 0 | m-D11 | tBu | H | A-22 | H | tBu |
| 11820 | 0 | m-D11 | tBu | H | A-23 | H | tBu |
| 11821 | 0 | m-D11 | tBu | H | A-24 | H | tBu |
| 11822 | 0 | m-D11 | tBu | H | A-25 | H | tBu |
| 11823 | 0 | m-D11 | tBu | H | A-26 | H | tBu |
| 11824 | 0 | m-D11 | tBu | H | A-27 | H | tBu |
| 11825 | 0 | m-D11 | tBu | H | A-28 | H | tBu |
| 11826 | 0 | m-D11 | tBu | H | A-29 | H | tBu |
| 11827 | 0 | m-D11 | tBu | H | A-30 | H | tBu |
| 11828 | 0 | m-D11 | tBu | H | A-31 | H | tBu |
| 11829 | 0 | m-D11 | tBu | H | A-32 | H | tBu |
| 11830 | 0 | m-D11 | tBu | H | A-33 | H | tBu |
| 11831 | 0 | m-D11 | tBu | H | A-34 | H | tBu |
| 11832 | 0 | m-D11 | tBu | H | A-35 | H | tBu |
| 11833 | 0 | m-D11 | tBu | H | A-36 | H | tBu |
| 11834 | 0 | m-D11 | tBu | H | A-37 | H | tBu |
| 11835 | 0 | m-D11 | tBu | H | A-38 | H | tBu |
| 11836 | 0 | m-D11 | tBu | H | A-39 | H | tBu |
| 11837 | 0 | m-D11 | tBu | H | A-40 | H | tBu |
| 11838 | 0 | m-D11 | tBu | H | A-41 | H | tBu |
| 11839 | 0 | m-D11 | tBu | H | A-42 | H | tBu |
| 11840 | 0 | m-D11 | tBu | H | A-43 | H | tBu |
| 11841 | 0 | m-D11 | tBu | H | A-44 | H | tBu |
| 11842 | 0 | m-D11 | tBu | H | A-45 | H | tBu |
| 11843 | 0 | m-D11 | tBu | H | A-46 | H | tBu |
| 11844 | 0 | m-D11 | tBu | H | A-47 | H | tBu |
| 11845 | 0 | m-D12 | tBu | H | A-1 | H | tBu |
| 11846 | 0 | m-D12 | tBu | H | A-2 | H | tBu |
| 11847 | 0 | m-D12 | tBu | H | A-3 | H | tBu |
| 11848 | 0 | m-D12 | tBu | H | A-4 | H | tBu |
| 11849 | 0 | m-D12 | tBu | H | A-5 | H | tBu |
| 11850 | 0 | m-D12 | tBu | H | A-6 | H | tBu |
| 11851 | 0 | m-D12 | tBu | H | A-7 | H | tBu |
| 11852 | 0 | m-D12 | tBu | H | A-8 | H | tBu |
| 11853 | 0 | m-D12 | tBu | H | A-9 | H | tBu |
| 11854 | 0 | m-D12 | tBu | H | A-10 | H | tBu |
| 11855 | 0 | m-D12 | tBu | H | A-11 | H | tBu |
| 11856 | 0 | m-D12 | tBu | H | A-12 | H | tBu |
| 11857 | 0 | m-D12 | tBu | H | A-13 | H | tBu |
| 11858 | 0 | m-D12 | tBu | H | A-14 | H | tBu |
| 11859 | 0 | m-D12 | tBu | H | A-15 | H | tBu |
| 11860 | 0 | m-D12 | tBu | H | A-16 | H | tBu |
| 11861 | 0 | m-D12 | tBu | H | A-17 | H | tBu |
| 11862 | 0 | m-D12 | tBu | H | A-18 | H | tBu |
| 11863 | 0 | m-D12 | tBu | H | A-19 | H | tBu |
| 11864 | 0 | m-D12 | tBu | H | A-20 | H | tBu |
| 11865 | 0 | m-D12 | tBu | H | A-21 | H | tBu |
| 11866 | 0 | m-D12 | tBu | H | A-22 | H | tBu |
| 11867 | 0 | m-D12 | tBu | H | A-23 | H | tBu |
| 11868 | 0 | m-D12 | tBu | H | A-24 | H | tBu |
| 11869 | 0 | m-D12 | tBu | H | A-25 | H | tBu |
| 11870 | 0 | m-D12 | tBu | H | A-26 | H | tBu |
| 11871 | 0 | m-D12 | tBu | H | A-27 | H | tBu |
| 11872 | 0 | m-D12 | tBu | H | A-28 | H | tBu |
| 11873 | 0 | m-D12 | tBu | H | A-29 | H | tBu |
| 11874 | 0 | m-D12 | tBu | H | A-30 | H | tBu |
| 11875 | 0 | m-D12 | tBu | H | A-31 | H | tBu |
| 11876 | 0 | m-D12 | tBu | H | A-32 | H | tBu |
| 11877 | 0 | m-D12 | tBu | H | A-33 | H | tBu |
| 11878 | 0 | m-D12 | tBu | H | A-34 | H | tBu |
| 11879 | 0 | m-D12 | tBu | H | A-35 | H | tBu |
| 11880 | 0 | m-D12 | tBu | H | A-36 | H | tBu |
| 11881 | 0 | m-D12 | tBu | H | A-37 | H | tBu |
| 11882 | 0 | m-D12 | tBu | H | A-38 | H | tBu |
| 11883 | 0 | m-D12 | tBu | H | A-39 | H | tBu |
| 11884 | 0 | m-D12 | tBu | H | A-40 | H | tBu |
| 11885 | 0 | m-D12 | tBu | H | A-41 | H | tBu |
| 11886 | 0 | m-D12 | tBu | H | A-42 | H | tBu |
| 11887 | 0 | m-D12 | tBu | H | A-43 | H | tBu |
| 11888 | 0 | m-D12 | tBu | H | A-44 | H | tBu |
| 11889 | 0 | m-D12 | tBu | H | A-45 | H | tBu |
| 11890 | 0 | m-D12 | tBu | H | A-46 | H | tBu |
| 11891 | 0 | m-D12 | tBu | H | A-47 | H | tBu |
| 11892 | 0 | m-D13 | tBu | H | A-1 | H | tBu |
| 11893 | 0 | m-D13 | tBu | H | A-2 | H | tBu |
| 11894 | 0 | m-D13 | tBu | H | A-3 | H | tBu |
| 11895 | 0 | m-D13 | tBu | H | A-4 | H | tBu |
| 11896 | 0 | m-D13 | tBu | H | A-5 | H | tBu |
| 11897 | 0 | m-D13 | tBu | H | A-6 | H | tBu |
| 11898 | 0 | m-D13 | tBu | H | A-7 | H | tBu |
| 11899 | 0 | m-D13 | tBu | H | A-8 | H | tBu |
| 11900 | 0 | m-D13 | tBu | H | A-9 | H | tBu |
| 11901 | 0 | m-D13 | tBu | H | A-10 | H | tBu |
| 11902 | 0 | m-D13 | tBu | H | A-11 | H | tBu |
| 11903 | 0 | m-D13 | tBu | H | A-12 | H | tBu |
| 11904 | 0 | m-D13 | tBu | H | A-13 | H | tBu |
| 11905 | 0 | m-D13 | tBu | H | A-14 | H | tBu |
| 11906 | 0 | m-D13 | tBu | H | A-15 | H | tBu |
| 11907 | 0 | m-D13 | tBu | H | A-16 | H | tBu |
| 11908 | 0 | m-D13 | tBu | H | A-17 | H | tBu |
| 11909 | 0 | m-D13 | tBu | H | A-18 | H | tBu |
| 11910 | 0 | m-D13 | tBu | H | A-19 | H | tBu |
| 11911 | 0 | m-D13 | tBu | H | A-20 | H | tBu |
| 11912 | 0 | m-D13 | tBu | H | A-21 | H | tBu |
| 11913 | 0 | m-D13 | tBu | H | A-22 | H | tBu |
| 11914 | 0 | m-D13 | tBu | H | A-23 | H | tBu |
| 11915 | 0 | m-D13 | tBu | H | A-24 | H | tBu |
| 11916 | 0 | m-D13 | tBu | H | A-25 | H | tBu |
| 11917 | 0 | m-D13 | tBu | H | A-26 | H | tBu |
| 11918 | 0 | m-D13 | tBu | H | A-27 | H | tBu |
| 11919 | 0 | m-D13 | tBu | H | A-28 | H | tBu |
| 11920 | 0 | m-D13 | tBu | H | A-29 | H | tBu |
| 11921 | 0 | m-D13 | tBu | H | A-30 | H | tBu |
| 11922 | 0 | m-D13 | tBu | H | A-31 | H | tBu |
| 11923 | 0 | m-D13 | tBu | H | A-32 | H | tBu |
| 11924 | 0 | m-D13 | tBu | H | A-33 | H | tBu |
| 11925 | 0 | m-D13 | tBu | H | A-34 | H | tBu |
| 11926 | 0 | m-D13 | tBu | H | A-35 | H | tBu |
| 11927 | 0 | m-D13 | tBu | H | A-36 | H | tBu |
| 11928 | 0 | m-D13 | tBu | H | A-37 | H | tBu |
| 11929 | 0 | m-D13 | tBu | H | A-38 | H | tBu |
| 11930 | 0 | m-D13 | tBu | H | A-39 | H | tBu |
| 11931 | 0 | m-D13 | tBu | H | A-40 | H | tBu |
| 11932 | 0 | m-D13 | tBu | H | A-41 | H | tBu |
| 11933 | 0 | m-D13 | tBu | H | A-42 | H | tBu |
| 11934 | 0 | m-D13 | tBu | H | A-43 | H | tBu |
| 11935 | 0 | m-D13 | tBu | H | A-44 | H | tBu |
| 11936 | 0 | m-D13 | tBu | H | A-45 | H | tBu |
| 11937 | 0 | m-D13 | tBu | H | A-46 | H | tBu |
| 11938 | 0 | m-D13 | tBu | H | A-47 | H | tBu |
| 11939 | 0 | m-D14 | tBu | H | A-1 | H | tBu |
| 11940 | 0 | m-D14 | tBu | H | A-2 | H | tBu |
| 11941 | 0 | m-D14 | tBu | H | A-3 | H | tBu |
| 11942 | 0 | m-D14 | tBu | H | A-4 | H | tBu |
| 11943 | 0 | m-D14 | tBu | H | A-5 | H | tBu |
| 11944 | 0 | m-D14 | tBu | H | A-6 | H | tBu |
| 11945 | 0 | m-D14 | tBu | H | A-7 | H | tBu |
| 11946 | 0 | m-D14 | tBu | H | A-8 | H | tBu |
| 11947 | 0 | m-D14 | tBu | H | A-9 | H | tBu |
| 11948 | 0 | m-D14 | tBu | H | A-10 | H | tBu |
| 11949 | 0 | m-D14 | tBu | H | A-11 | H | tBu |
| 11950 | 0 | m-D14 | tBu | H | A-12 | H | tBu |
| 11951 | 0 | m-D14 | tBu | H | A-13 | H | tBu |
| 11952 | 0 | m-D14 | tBu | H | A-14 | H | tBu |
| 11953 | 0 | m-D14 | tBu | H | A-15 | H | tBu |
| 11954 | 0 | m-D14 | tBu | H | A-16 | H | tBu |
| 11955 | 0 | m-D14 | tBu | H | A-17 | H | tBu |
| 11956 | 0 | m-D14 | tBu | H | A-18 | H | tBu |
| 11957 | 0 | m-D14 | tBu | H | A-19 | H | tBu |
| 11958 | 0 | m-D14 | tBu | H | A-20 | H | tBu |
| 11959 | 0 | m-D14 | tBu | H | A-21 | H | tBu |
| 11960 | 0 | m-D14 | tBu | H | A-22 | H | tBu |
| 11961 | 0 | m-D14 | tBu | H | A-23 | H | tBu |
| 11962 | 0 | m-D14 | tBu | H | A-24 | H | tBu |
| 11963 | 0 | m-D14 | tBu | H | A-25 | H | tBu |
| 11964 | 0 | m-D14 | tBu | H | A-26 | H | tBu |
| 11965 | 0 | m-D14 | tBu | H | A-27 | H | tBu |
| 11966 | 0 | m-D14 | tBu | H | A-28 | H | tBu |
| 11967 | 0 | m-D14 | tBu | H | A-29 | H | tBu |
| 11968 | 0 | m-D14 | tBu | H | A-30 | H | tBu |
| 11969 | 0 | m-D14 | tBu | H | A-31 | H | tBu |
| 11970 | 0 | m-D14 | tBu | H | A-32 | H | tBu |
| 11971 | 0 | m-D14 | tBu | H | A-33 | H | tBu |
| 11972 | 0 | m-D14 | tBu | H | A-34 | H | tBu |
| 11973 | 0 | m-D14 | tBu | H | A-35 | H | tBu |
| 11974 | 0 | m-D14 | tBu | H | A-36 | H | tBu |
| 11975 | 0 | m-D14 | tBu | H | A-37 | H | tBu |
| 11976 | 0 | m-D14 | tBu | H | A-38 | H | tBu |
| 11977 | 0 | m-D14 | tBu | H | A-39 | H | tBu |
| 11978 | 0 | m-D14 | tBu | H | A-40 | H | tBu |
| 11979 | 0 | m-D14 | tBu | H | A-41 | H | tBu |
| 11980 | 0 | m-D14 | tBu | H | A-42 | H | tBu |
| 11981 | 0 | m-D14 | tBu | H | A-43 | H | tBu |
| 11982 | 0 | m-D14 | tBu | H | A-44 | H | tBu |
| 11983 | 0 | m-D14 | tBu | H | A-45 | H | tBu |
| 11984 | 0 | m-D14 | tBu | H | A-46 | H | tBu |
| 11985 | 0 | m-D14 | tBu | H | A-47 | H | tBu |
| 11986 | 0 | m-D1 | H | tBu | A-1 | tBu | H |
| 11987 | 0 | m-D1 | H | tBu | A-2 | tBu | H |
| 11988 | 0 | m-D1 | H | tBu | A-3 | tBu | H |
| 11989 | 0 | m-D1 | H | tBu | A-4 | tBu | H |
| 11990 | 0 | m-D1 | H | tBu | A-5 | tBu | H |
| 11991 | 0 | m-D1 | H | tBu | A-6 | tBu | H |
| 11992 | 0 | m-D1 | H | tBu | A-7 | tBu | H |
| 11993 | 0 | m-D1 | H | tBu | A-8 | tBu | H |
| 11994 | 0 | m-D1 | H | tBu | A-9 | tBu | H |
| 11995 | 0 | m-D1 | H | tBu | A-10 | tBu | H |
| 11996 | 0 | m-D1 | H | tBu | A-11 | tBu | H |
| 11997 | 0 | m-D1 | H | tBu | A-12 | tBu | H |
| 11998 | 0 | m-D1 | H | tBu | A-13 | tBu | H |
| 11999 | 0 | m-D1 | H | tBu | A-14 | tBu | H |
| 12000 | 0 | m-D1 | H | tBu | A-15 | tBu | H |
| 12001 | 0 | m-D1 | H | tBu | A-16 | tBu | H |
| 12002 | 0 | m-D1 | H | tBu | A-17 | tBu | H |
| 12003 | 0 | m-D1 | H | tBu | A-18 | tBu | H |
| 12004 | 0 | m-D1 | H | tBu | A-19 | tBu | H |
| 12005 | 0 | m-D1 | H | tBu | A-20 | tBu | H |
| 12006 | 0 | m-D1 | H | tBu | A-21 | tBu | H |
| 12007 | 0 | m-D1 | H | tBu | A-22 | tBu | H |
| 12008 | 0 | m-D1 | H | tBu | A-23 | tBu | H |
| 12009 | 0 | m-D1 | H | tBu | A-24 | tBu | H |
| 12010 | 0 | m-D1 | H | tBu | A-25 | tBu | H |
| 12011 | 0 | m-D1 | H | tBu | A-26 | tBu | H |
| 12012 | 0 | m-D1 | H | tBu | A-27 | tBu | H |
| 12013 | 0 | m-D1 | H | tBu | A-28 | tBu | H |
| 12014 | 0 | m-D1 | H | tBu | A-29 | tBu | H |
| 12015 | 0 | m-D1 | H | tBu | A-30 | tBu | H |
| 12016 | 0 | m-D1 | H | tBu | A-31 | tBu | H |
| 12017 | 0 | m-D1 | H | tBu | A-32 | tBu | H |
| 12018 | 0 | m-D1 | H | tBu | A-33 | tBu | H |
| 12019 | 0 | m-D1 | H | tBu | A-34 | tBu | H |
| 12020 | 0 | m-D1 | H | tBu | A-35 | tBu | H |
| 12021 | 0 | m-D1 | H | tBu | A-36 | tBu | H |
| 12022 | 0 | m-D1 | H | tBu | A-37 | tBu | H |
| 12023 | 0 | m-D1 | H | tBu | A-38 | tBu | H |
| 12024 | 0 | m-D1 | H | tBu | A-39 | tBu | H |
| 12025 | 0 | m-D1 | H | tBu | A-40 | tBu | H |
| 12026 | 0 | m-D1 | H | tBu | A-41 | tBu | H |
| 12027 | 0 | m-D1 | H | tBu | A-42 | tBu | H |
| 12028 | 0 | m-D1 | H | tBu | A-43 | tBu | H |
| 12029 | 0 | m-D1 | H | tBu | A-44 | tBu | H |
| 12030 | 0 | m-D1 | H | tBu | A-45 | tBu | H |
| 12031 | 0 | m-D1 | H | tBu | A-46 | tBu | H |
| 12032 | 0 | m-D1 | H | tBu | A-47 | tBu | H |
| 12033 | 0 | m-D2 | H | tBu | A-1 | tBu | H |
| 12034 | 0 | m-D2 | H | tBu | A-2 | tBu | H |
| 12035 | 0 | m-D2 | H | tBu | A-3 | tBu | H |
| 12036 | 0 | m-D2 | H | tBu | A-4 | tBu | H |
| 12037 | 0 | m-D2 | H | tBu | A-5 | tBu | H |
| 12038 | 0 | m-D2 | H | tBu | A-6 | tBu | H |
| 12039 | 0 | m-D2 | H | tBu | A-7 | tBu | H |
| 12040 | 0 | m-D2 | H | tBu | A-8 | tBu | H |
| 12041 | 0 | m-D2 | H | tBu | A-9 | tBu | H |
| 12042 | 0 | m-D2 | H | tBu | A-10 | tBu | H |
| 12043 | 0 | m-D2 | H | tBu | A-11 | tBu | H |
| 12044 | 0 | m-D2 | H | tBu | A-12 | tBu | H |
| 12045 | 0 | m-D2 | H | tBu | A-13 | tBu | H |
| 12046 | 0 | m-D2 | H | tBu | A-14 | tBu | H |
| 12047 | 0 | m-D2 | H | tBu | A-15 | tBu | H |
| 12048 | 0 | m-D2 | H | tBu | A-16 | tBu | H |
| 12049 | 0 | m-D2 | H | tBu | A-17 | tBu | H |
| 12050 | 0 | m-D2 | H | tBu | A-18 | tBu | H |
| 12051 | 0 | m-D2 | H | tBu | A-19 | tBu | H |
| 12052 | 0 | m-D2 | H | tBu | A-20 | tBu | H |
| 12053 | 0 | m-D2 | H | tBu | A-21 | tBu | H |
| 12054 | 0 | m-D2 | H | tBu | A-22 | tBu | H |
| 12055 | 0 | m-D2 | H | tBu | A-23 | tBu | H |
| 12056 | 0 | m-D2 | H | tBu | A-24 | tBu | H |
| 12057 | 0 | m-D2 | H | tBu | A-25 | tBu | H |
| 12058 | 0 | m-D2 | H | tBu | A-26 | tBu | H |
| 12059 | 0 | m-D2 | H | tBu | A-27 | tBu | H |
| 12060 | 0 | m-D2 | H | tBu | A-28 | tBu | H |
| 12061 | 0 | m-D2 | H | tBu | A-29 | tBu | H |
| 12062 | 0 | m-D2 | H | tBu | A-30 | tBu | H |
| 12063 | 0 | m-D2 | H | tBu | A-31 | tBu | H |
| 12064 | 0 | m-D2 | H | tBu | A-32 | tBu | H |
| 12065 | 0 | m-D2 | H | tBu | A-33 | tBu | H |
| 12066 | 0 | m-D2 | H | tBu | A-34 | tBu | H |
| 12067 | 0 | m-D2 | H | tBu | A-35 | tBu | H |
| 12068 | 0 | m-D2 | H | tBu | A-36 | tBu | H |
| 12069 | 0 | m-D2 | H | tBu | A-37 | tBu | H |
| 12070 | 0 | m-D2 | H | tBu | A-38 | tBu | H |
| 12071 | 0 | m-D2 | H | tBu | A-39 | tBu | H |
| 12072 | 0 | m-D2 | H | tBu | A-40 | tBu | H |
| 12073 | 0 | m-D2 | H | tBu | A-41 | tBu | H |
| 12074 | 0 | m-D2 | H | tBu | A-42 | tBu | H |
| 12075 | 0 | m-D2 | H | tBu | A-43 | tBu | H |
| 12076 | 0 | m-D2 | H | tBu | A-44 | tBu | H |
| 12077 | 0 | m-D2 | H | tBu | A-45 | tBu | H |
| 12078 | 0 | m-D2 | H | tBu | A-46 | tBu | H |
| 12079 | 0 | m-D2 | H | tBu | A-47 | tBu | H |
| 12080 | 0 | m-D3 | H | tBu | A-1 | tBu | H |
| 12081 | 0 | m-D3 | H | tBu | A-2 | tBu | H |
| 12082 | 0 | m-D3 | H | tBu | A-3 | tBu | H |
| 12083 | 0 | m-D3 | H | tBu | A-4 | tBu | H |
| 12084 | 0 | m-D3 | H | tBu | A-5 | tBu | H |
| 12085 | 0 | m-D3 | H | tBu | A-6 | tBu | H |
| 12086 | 0 | m-D3 | H | tBu | A-7 | tBu | H |
| 12087 | 0 | m-D3 | H | tBu | A-8 | tBu | H |
| 12088 | 0 | m-D3 | H | tBu | A-9 | tBu | H |
| 12089 | 0 | m-D3 | H | tBu | A-10 | tBu | H |
| 12090 | 0 | m-D3 | H | tBu | A-11 | tBu | H |
| 12091 | 0 | m-D3 | H | tBu | A-12 | tBu | H |
| 12092 | 0 | m-D3 | H | tBu | A-13 | tBu | H |
| 12093 | 0 | m-D3 | H | tBu | A-14 | tBu | H |
| 12094 | 0 | m-D3 | H | tBu | A-15 | tBu | H |
| 12095 | 0 | m-D3 | H | tBu | A-16 | tBu | H |
| 12096 | 0 | m-D3 | H | tBu | A-17 | tBu | H |
| 12097 | 0 | m-D3 | H | tBu | A-18 | tBu | H |
| 12098 | 0 | m-D3 | H | tBu | A-19 | tBu | H |
| 12099 | 0 | m-D3 | H | tBu | A-20 | tBu | H |
| 12100 | 0 | m-D3 | H | tBu | A-21 | tBu | H |
| 12101 | 0 | m-D3 | H | tBu | A-22 | tBu | H |
| 12102 | 0 | m-D3 | H | tBu | A-23 | tBu | H |
| 12103 | 0 | m-D3 | H | tBu | A-24 | tBu | H |
| 12104 | 0 | m-D3 | H | tBu | A-25 | tBu | H |
| 12105 | 0 | m-D3 | H | tBu | A-26 | tBu | H |
| 12106 | 0 | m-D3 | H | tBu | A-27 | tBu | H |
| 12107 | 0 | m-D3 | H | tBu | A-28 | tBu | H |
| 12108 | 0 | m-D3 | H | tBu | A-29 | tBu | H |
| 12109 | 0 | m-D3 | H | tBu | A-30 | tBu | H |
| 12110 | 0 | m-D3 | H | tBu | A-31 | tBu | H |
| 12111 | 0 | m-D3 | H | tBu | A-32 | tBu | H |
| 12112 | 0 | m-D3 | H | tBu | A-33 | tBu | H |
| 12113 | 0 | m-D3 | H | tBu | A-34 | tBu | H |
| 12114 | 0 | m-D3 | H | tBu | A-35 | tBu | H |
| 12115 | 0 | m-D3 | H | tBu | A-36 | tBu | H |
| 12116 | 0 | m-D3 | H | tBu | A-37 | tBu | H |
| 12117 | 0 | m-D3 | H | tBu | A-38 | tBu | H |
| 12118 | 0 | m-D3 | H | tBu | A-39 | tBu | H |
| 12119 | 0 | m-D3 | H | tBu | A-40 | tBu | H |
| 12120 | 0 | m-D3 | H | tBu | A-41 | tBu | H |
| 12121 | 0 | m-D3 | H | tBu | A-42 | tBu | H |
| 12122 | 0 | m-D3 | H | tBu | A-43 | tBu | H |
| 12123 | 0 | m-D3 | H | tBu | A-44 | tBu | H |
| 12124 | 0 | m-D3 | H | tBu | A-45 | tBu | H |
| 12125 | 0 | m-D3 | H | tBu | A-46 | tBu | H |
| 12126 | 0 | m-D3 | H | tBu | A-47 | tBu | H |
| 12127 | 0 | m-D4 | H | tBu | A-1 | tBu | H |
| 12128 | 0 | m-D4 | H | tBu | A-2 | tBu | H |
| 12129 | 0 | m-D4 | H | tBu | A-3 | tBu | H |
| 12130 | 0 | m-D4 | H | tBu | A-4 | tBu | H |
| 12131 | 0 | m-D4 | H | tBu | A-5 | tBu | H |
| 12132 | 0 | m-D4 | H | tBu | A-6 | tBu | H |
| 12133 | 0 | m-D4 | H | tBu | A-7 | tBu | H |
| 12134 | 0 | m-D4 | H | tBu | A-8 | tBu | H |
| 12135 | 0 | m-D4 | H | tBu | A-9 | tBu | H |
| 12136 | 0 | m-D4 | H | tBu | A-10 | tBu | H |
| 12137 | 0 | m-D4 | H | tBu | A-11 | tBu | H |
| 12138 | 0 | m-D4 | H | tBu | A-12 | tBu | H |
| 12139 | 0 | m-D4 | H | tBu | A-13 | tBu | H |
| 12140 | 0 | m-D4 | H | tBu | A-14 | tBu | H |
| 12141 | 0 | m-D4 | H | tBu | A-15 | tBu | H |
| 12142 | 0 | m-D4 | H | tBu | A-16 | tBu | H |
| 12143 | 0 | m-D4 | H | tBu | A-17 | tBu | H |
| 12144 | 0 | m-D4 | H | tBu | A-18 | tBu | H |
| 12145 | 0 | m-D4 | H | tBu | A-19 | tBu | H |
| 12146 | 0 | m-D4 | H | tBu | A-20 | tBu | H |
| 12147 | 0 | m-D4 | H | tBu | A-21 | tBu | H |
| 12148 | 0 | m-D4 | H | tBu | A-22 | tBu | H |
| 12149 | 0 | m-D4 | H | tBu | A-23 | tBu | H |
| 12150 | 0 | m-D4 | H | tBu | A-24 | tBu | H |
| 12151 | 0 | m-D4 | H | tBu | A-25 | tBu | H |
| 12152 | 0 | m-D4 | H | tBu | A-26 | tBu | H |
| 12153 | 0 | m-D4 | H | tBu | A-27 | tBu | H |
| 12154 | 0 | m-D4 | H | tBu | A-28 | tBu | H |
| 12155 | 0 | m-D4 | H | tBu | A-29 | tBu | H |
| 12156 | 0 | m-D4 | H | tBu | A-30 | tBu | H |
| 12157 | 0 | m-D4 | H | tBu | A-31 | tBu | H |
| 12158 | 0 | m-D4 | H | tBu | A-32 | tBu | H |
| 12159 | 0 | m-D4 | H | tBu | A-33 | tBu | H |
| 12160 | 0 | m-D4 | H | tBu | A-34 | tBu | H |
| 12161 | 0 | m-D4 | H | tBu | A-35 | tBu | H |
| 12162 | 0 | m-D4 | H | tBu | A-36 | tBu | H |
| 12163 | 0 | m-D4 | H | tBu | A-37 | tBu | H |
| 12164 | 0 | m-D4 | H | tBu | A-38 | tBu | H |
| 12165 | 0 | m-D4 | H | tBu | A-39 | tBu | H |
| 12166 | 0 | m-D4 | H | tBu | A-40 | tBu | H |
| 12167 | 0 | m-D4 | H | tBu | A-41 | tBu | H |
| 12168 | 0 | m-D4 | H | tBu | A-42 | tBu | H |
| 12169 | 0 | m-D4 | H | tBu | A-43 | tBu | H |
| 12170 | 0 | m-D4 | H | tBu | A-44 | tBu | H |
| 12171 | 0 | m-D4 | H | tBu | A-45 | tBu | H |
| 12172 | 0 | m-D4 | H | tBu | A-46 | tBu | H |
| 12173 | 0 | m-D4 | H | tBu | A-47 | tBu | H |
| 12174 | 0 | m-D5 | H | tBu | A-1 | tBu | H |
| 12175 | 0 | m-D5 | H | tBu | A-2 | tBu | H |
| 12176 | 0 | m-D5 | H | tBu | A-3 | tBu | H |
| 12177 | 0 | m-D5 | H | tBu | A-4 | tBu | H |
| 12178 | 0 | m-D5 | H | tBu | A-5 | tBu | H |
| 12179 | 0 | m-D5 | H | tBu | A-6 | tBu | H |
| 12180 | 0 | m-D5 | H | tBu | A-7 | tBu | H |
| 12181 | 0 | m-D5 | H | tBu | A-8 | tBu | H |
| 12182 | 0 | m-D5 | H | tBu | A-9 | tBu | H |
| 12183 | 0 | m-D5 | H | tBu | A-10 | tBu | H |
| 12184 | 0 | m-D5 | H | tBu | A-11 | tBu | H |
| 12185 | 0 | m-D5 | H | tBu | A-12 | tBu | H |
| 12186 | 0 | m-D5 | H | tBu | A-13 | tBu | H |
| 12187 | 0 | m-D5 | H | tBu | A-14 | tBu | H |
| 12188 | 0 | m-D5 | H | tBu | A-15 | tBu | H |
| 12189 | 0 | m-D5 | H | tBu | A-16 | tBu | H |
| 12190 | 0 | m-D5 | H | tBu | A-17 | tBu | H |
| 12191 | 0 | m-D5 | H | tBu | A-18 | tBu | H |
| 12192 | 0 | m-D5 | H | tBu | A-19 | tBu | H |
| 12193 | 0 | m-D5 | H | tBu | A-20 | tBu | H |
| 12194 | 0 | m-D5 | H | tBu | A-21 | tBu | H |
| 12195 | 0 | m-D5 | H | tBu | A-22 | tBu | H |
| 12196 | 0 | m-D5 | H | tBu | A-23 | tBu | H |
| 12197 | 0 | m-D5 | H | tBu | A-24 | tBu | H |
| 12198 | 0 | m-D5 | H | tBu | A-25 | tBu | H |
| 12199 | 0 | m-D5 | H | tBu | A-26 | tBu | H |
| 12200 | 0 | m-D5 | H | tBu | A-27 | tBu | H |
| 12201 | 0 | m-D5 | H | tBu | A-28 | tBu | H |
| 12202 | 0 | m-D5 | H | tBu | A-29 | tBu | H |
| 12203 | 0 | m-D5 | H | tBu | A-30 | tBu | H |
| 12204 | 0 | m-D5 | H | tBu | A-31 | tBu | H |
| 12205 | 0 | m-D5 | H | tBu | A-32 | tBu | H |
| 12206 | 0 | m-D5 | H | tBu | A-33 | tBu | H |
| 12207 | 0 | m-D5 | H | tBu | A-34 | tBu | H |
| 12208 | 0 | m-D5 | H | tBu | A-35 | tBu | H |
| 12209 | 0 | m-D5 | H | tBu | A-36 | tBu | H |
| 12210 | 0 | m-D5 | H | tBu | A-37 | tBu | H |
| 12211 | 0 | m-D5 | H | tBu | A-38 | tBu | H |
| 12212 | 0 | m-D5 | H | tBu | A-39 | tBu | H |
| 12213 | 0 | m-D5 | H | tBu | A-40 | tBu | H |
| 12214 | 0 | m-D5 | H | tBu | A-41 | tBu | H |
| 12215 | 0 | m-D5 | H | tBu | A-42 | tBu | H |
| 12216 | 0 | m-D5 | H | tBu | A-43 | tBu | H |
| 12217 | 0 | m-D5 | H | tBu | A-44 | tBu | H |
| 12218 | 0 | m-D5 | H | tBu | A-45 | tBu | H |
| 12219 | 0 | m-D5 | H | tBu | A-46 | tBu | H |
| 12220 | 0 | m-D5 | H | tBu | A-47 | tBu | H |
| 12221 | 0 | m-D6 | H | tBu | A-1 | tBu | H |
| 12222 | 0 | m-D6 | H | tBu | A-2 | tBu | H |
| 12223 | 0 | m-D6 | H | tBu | A-3 | tBu | H |
| 12224 | 0 | m-D6 | H | tBu | A-4 | tBu | H |
| 12225 | 0 | m-D6 | H | tBu | A-5 | tBu | H |
| 12226 | 0 | m-D6 | H | tBu | A-6 | tBu | H |
| 12227 | 0 | m-D6 | H | tBu | A-7 | tBu | H |
| 12228 | 0 | m-D6 | H | tBu | A-8 | tBu | H |
| 12229 | 0 | m-D6 | H | tBu | A-9 | tBu | H |
| 12230 | 0 | m-D6 | H | tBu | A-10 | tBu | H |
| 12231 | 0 | m-D6 | H | tBu | A-11 | tBu | H |
| 12232 | 0 | m-D6 | H | tBu | A-12 | tBu | H |
| 12233 | 0 | m-D6 | H | tBu | A-13 | tBu | H |
| 12234 | 0 | m-D6 | H | tBu | A-14 | tBu | H |
| 12235 | 0 | m-D6 | H | tBu | A-15 | tBu | H |
| 12236 | 0 | m-D6 | H | tBu | A-16 | tBu | H |
| 12237 | 0 | m-D6 | H | tBu | A-17 | tBu | H |
| 12238 | 0 | m-D6 | H | tBu | A-18 | tBu | H |
| 12239 | 0 | m-D6 | H | tBu | A-19 | tBu | H |
| 12240 | 0 | m-D6 | H | tBu | A-20 | tBu | H |
| 12241 | 0 | m-D6 | H | tBu | A-21 | tBu | H |
| 12242 | 0 | m-D6 | H | tBu | A-22 | tBu | H |
| 12243 | 0 | m-D6 | H | tBu | A-23 | tBu | H |
| 12244 | 0 | m-D6 | H | tBu | A-24 | tBu | H |
| 12245 | 0 | m-D6 | H | tBu | A-25 | tBu | H |
| 12246 | 0 | m-D6 | H | tBu | A-26 | tBu | H |
| 12247 | 0 | m-D6 | H | tBu | A-27 | tBu | H |
| 12248 | 0 | m-D6 | H | tBu | A-28 | tBu | H |
| 12249 | 0 | m-D6 | H | tBu | A-29 | tBu | H |
| 12250 | 0 | m-D6 | H | tBu | A-30 | tBu | H |
| 12251 | 0 | m-D6 | H | tBu | A-31 | tBu | H |
| 12252 | 0 | m-D6 | H | tBu | A-32 | tBu | H |
| 12253 | 0 | m-D6 | H | tBu | A-33 | tBu | H |
| 12254 | 0 | m-D6 | H | tBu | A-34 | tBu | H |
| 12255 | 0 | m-D6 | H | tBu | A-35 | tBu | H |
| 12256 | 0 | m-D6 | H | tBu | A-36 | tBu | H |
| 12257 | 0 | m-D6 | H | tBu | A-37 | tBu | H |
| 12258 | 0 | m-D6 | H | tBu | A-38 | tBu | H |
| 12259 | 0 | m-D6 | H | tBu | A-39 | tBu | H |
| 12260 | 0 | m-D6 | H | tBu | A-40 | tBu | H |
| 12261 | 0 | m-D6 | H | tBu | A-41 | tBu | H |
| 12262 | 0 | m-D6 | H | tBu | A-42 | tBu | H |
| 12263 | 0 | m-D6 | H | tBu | A-43 | tBu | H |
| 12264 | 0 | m-D6 | H | tBu | A-44 | tBu | H |
| 12265 | 0 | m-D6 | H | tBu | A-45 | tBu | H |
| 12266 | 0 | m-D6 | H | tBu | A-46 | tBu | H |
| 12267 | 0 | m-D6 | H | tBu | A-47 | tBu | H |
| 12268 | 0 | m-D7 | H | tBu | A-1 | tBu | H |
| 12269 | 0 | m-D7 | H | tBu | A-2 | tBu | H |
| 12270 | 0 | m-D7 | H | tBu | A-3 | tBu | H |
| 12271 | 0 | m-D7 | H | tBu | A-4 | tBu | H |
| 12272 | 0 | m-D7 | H | tBu | A-5 | tBu | H |
| 12273 | 0 | m-D7 | H | tBu | A-6 | tBu | H |
| 12274 | 0 | m-D7 | H | tBu | A-7 | tBu | H |
| 12275 | 0 | m-D7 | H | tBu | A-8 | tBu | H |
| 12276 | 0 | m-D7 | H | tBu | A-9 | tBu | H |
| 12277 | 0 | m-D7 | H | tBu | A-10 | tBu | H |
| 12278 | 0 | m-D7 | H | tBu | A-11 | tBu | H |
| 12279 | 0 | m-D7 | H | tBu | A-12 | tBu | H |
| 12280 | 0 | m-D7 | H | tBu | A-13 | tBu | H |
| 12281 | 0 | m-D7 | H | tBu | A-14 | tBu | H |
| 12282 | 0 | m-D7 | H | tBu | A-15 | tBu | H |
| 12283 | 0 | m-D7 | H | tBu | A-16 | tBu | H |
| 12284 | 0 | m-D7 | H | tBu | A-17 | tBu | H |
| 12285 | 0 | m-D7 | H | tBu | A-18 | tBu | H |
| 12286 | 0 | m-D7 | H | tBu | A-19 | tBu | H |
| 12287 | 0 | m-D7 | H | tBu | A-20 | tBu | H |
| 12288 | 0 | m-D7 | H | tBu | A-21 | tBu | H |
| 12289 | 0 | m-D7 | H | tBu | A-22 | tBu | H |
| 12290 | 0 | m-D7 | H | tBu | A-23 | tBu | H |
| 12291 | 0 | m-D7 | H | tBu | A-24 | tBu | H |
| 12292 | 0 | m-D7 | H | tBu | A-25 | tBu | H |
| 12293 | 0 | m-D7 | H | tBu | A-26 | tBu | H |
| 12294 | 0 | m-D7 | H | tBu | A-27 | tBu | H |
| 12295 | 0 | m-D7 | H | tBu | A-28 | tBu | H |
| 12296 | 0 | m-D7 | H | tBu | A-29 | tBu | H |
| 12297 | 0 | m-D7 | H | tBu | A-30 | tBu | H |
| 12298 | 0 | m-D7 | H | tBu | A-31 | tBu | H |
| 12299 | 0 | m-D7 | H | tBu | A-32 | tBu | H |
| 12300 | 0 | m-D7 | H | tBu | A-33 | tBu | H |
| 12301 | 0 | m-D7 | H | tBu | A-34 | tBu | H |
| 12302 | 0 | m-D7 | H | tBu | A-35 | tBu | H |
| 12303 | 0 | m-D7 | H | tBu | A-36 | tBu | H |
| 12304 | 0 | m-D7 | H | tBu | A-37 | tBu | H |
| 12305 | 0 | m-D7 | H | tBu | A-38 | tBu | H |
| 12306 | 0 | m-D7 | H | tBu | A-39 | tBu | H |
| 12307 | 0 | m-D7 | H | tBu | A-40 | tBu | H |
| 12308 | 0 | m-D7 | H | tBu | A-41 | tBu | H |
| 12309 | 0 | m-D7 | H | tBu | A-42 | tBu | H |
| 12310 | 0 | m-D7 | H | tBu | A-43 | tBu | H |
| 12311 | 0 | m-D7 | H | tBu | A-44 | tBu | H |
| 12312 | 0 | m-D7 | H | tBu | A-45 | tBu | H |
| 12313 | 0 | m-D7 | H | tBu | A-46 | tBu | H |
| 12314 | 0 | m-D7 | H | tBu | A-47 | tBu | H |
| 12315 | 0 | m-D8 | H | tBu | A-1 | tBu | H |
| 12316 | 0 | m-D8 | H | tBu | A-2 | tBu | H |
| 12317 | 0 | m-D8 | H | tBu | A-3 | tBu | H |
| 12318 | 0 | m-D8 | H | tBu | A-4 | tBu | H |
| 12319 | 0 | m-D8 | H | tBu | A-5 | tBu | H |
| 12320 | 0 | m-D8 | H | tBu | A-6 | tBu | H |
| 12321 | 0 | m-D8 | H | tBu | A-7 | tBu | H |
| 12322 | 0 | m-D8 | H | tBu | A-8 | tBu | H |
| 12323 | 0 | m-D8 | H | tBu | A-9 | tBu | H |
| 12324 | 0 | m-D8 | H | tBu | A-10 | tBu | H |
| 12325 | 0 | m-D8 | H | tBu | A-11 | tBu | H |
| 12326 | 0 | m-D8 | H | tBu | A-12 | tBu | H |
| 12327 | 0 | m-D8 | H | tBu | A-13 | tBu | H |
| 12328 | 0 | m-D8 | H | tBu | A-14 | tBu | H |
| 12329 | 0 | m-D8 | H | tBu | A-15 | tBu | H |
| 12330 | 0 | m-D8 | H | tBu | A-16 | tBu | H |
| 12331 | 0 | m-D8 | H | tBu | A-17 | tBu | H |
| 12332 | 0 | m-D8 | H | tBu | A-18 | tBu | H |
| 12333 | 0 | m-D8 | H | tBu | A-19 | tBu | H |
| 12334 | 0 | m-D8 | H | tBu | A-20 | tBu | H |
| 12335 | 0 | m-D8 | H | tBu | A-21 | tBu | H |
| 12336 | 0 | m-D8 | H | tBu | A-22 | tBu | H |
| 12337 | 0 | m-D8 | H | tBu | A-23 | tBu | H |
| 12338 | 0 | m-D8 | H | tBu | A-24 | tBu | H |
| 12339 | 0 | m-D8 | H | tBu | A-25 | tBu | H |
| 12340 | 0 | m-D8 | H | tBu | A-26 | tBu | H |
| 12341 | 0 | m-D8 | H | tBu | A-27 | tBu | H |
| 12342 | 0 | m-D8 | H | tBu | A-28 | tBu | H |
| 12343 | 0 | m-D8 | H | tBu | A-29 | tBu | H |
| 12344 | 0 | m-D8 | H | tBu | A-30 | tBu | H |
| 12345 | 0 | m-D8 | H | tBu | A-31 | tBu | H |
| 12346 | 0 | m-D8 | H | tBu | A-32 | tBu | H |
| 12347 | 0 | m-D8 | H | tBu | A-33 | tBu | H |
| 12348 | 0 | m-D8 | H | tBu | A-34 | tBu | H |
| 12349 | 0 | m-D8 | H | tBu | A-35 | tBu | H |
| 12350 | 0 | m-D8 | H | tBu | A-36 | tBu | H |
| 12351 | 0 | m-D8 | H | tBu | A-37 | tBu | H |
| 12352 | 0 | m-D8 | H | tBu | A-38 | tBu | H |
| 12353 | 0 | m-D8 | H | tBu | A-39 | tBu | H |
| 12354 | 0 | m-D8 | H | tBu | A-40 | tBu | H |
| 12355 | 0 | m-D8 | H | tBu | A-41 | tBu | H |
| 12356 | 0 | m-D8 | H | tBu | A-42 | tBu | H |
| 12357 | 0 | m-D8 | H | tBu | A-43 | tBu | H |
| 12358 | 0 | m-D8 | H | tBu | A-44 | tBu | H |
| 12359 | 0 | m-D8 | H | tBu | A-45 | tBu | H |
| 12360 | 0 | m-D8 | H | tBu | A-46 | tBu | H |
| 12361 | 0 | m-D8 | H | tBu | A-47 | tBu | H |
| 12362 | 0 | m-D9 | H | tBu | A-1 | tBu | H |
| 12363 | 0 | m-D9 | H | tBu | A-2 | tBu | H |
| 12364 | 0 | m-D9 | H | tBu | A-3 | tBu | H |
| 12365 | 0 | m-D9 | H | tBu | A-4 | tBu | H |
| 12366 | 0 | m-D9 | H | tBu | A-5 | tBu | H |
| 12367 | 0 | m-D9 | H | tBu | A-6 | tBu | H |
| 12368 | 0 | m-D9 | H | tBu | A-7 | tBu | H |
| 12369 | 0 | m-D9 | H | tBu | A-8 | tBu | H |
| 12370 | 0 | m-D9 | H | tBu | A-9 | tBu | H |
| 12371 | 0 | m-D9 | H | tBu | A-10 | tBu | H |
| 12372 | 0 | m-D9 | H | tBu | A-11 | tBu | H |
| 12373 | 0 | m-D9 | H | tBu | A-12 | tBu | H |
| 12374 | 0 | m-D9 | H | tBu | A-13 | tBu | H |
| 12375 | 0 | m-D9 | H | tBu | A-14 | tBu | H |
| 12376 | 0 | m-D9 | H | tBu | A-15 | tBu | H |
| 12377 | 0 | m-D9 | H | tBu | A-16 | tBu | H |
| 12378 | 0 | m-D9 | H | tBu | A-17 | tBu | H |
| 12379 | 0 | m-D9 | H | tBu | A-18 | tBu | H |
| 12380 | 0 | m-D9 | H | tBu | A-19 | tBu | H |
| 12381 | 0 | m-D9 | H | tBu | A-20 | tBu | H |
| 12382 | 0 | m-D9 | H | tBu | A-21 | tBu | H |
| 12383 | 0 | m-D9 | H | tBu | A-22 | tBu | H |
| 12384 | 0 | m-D9 | H | tBu | A-23 | tBu | H |
| 12385 | 0 | m-D9 | H | tBu | A-24 | tBu | H |
| 12386 | 0 | m-D9 | H | tBu | A-25 | tBu | H |
| 12387 | 0 | m-D9 | H | tBu | A-26 | tBu | H |
| 12388 | 0 | m-D9 | H | tBu | A-27 | tBu | H |
| 12389 | 0 | m-D9 | H | tBu | A-28 | tBu | H |
| 12390 | 0 | m-D9 | H | tBu | A-29 | tBu | H |
| 12391 | 0 | m-D9 | H | tBu | A-30 | tBu | H |
| 12392 | 0 | m-D9 | H | tBu | A-31 | tBu | H |
| 12393 | 0 | m-D9 | H | tBu | A-32 | tBu | H |
| 12394 | 0 | m-D9 | H | tBu | A-33 | tBu | H |
| 12395 | 0 | m-D9 | H | tBu | A-34 | tBu | H |
| 12396 | 0 | m-D9 | H | tBu | A-35 | tBu | H |
| 12397 | 0 | m-D9 | H | tBu | A-36 | tBu | H |
| 12398 | 0 | m-D9 | H | tBu | A-37 | tBu | H |
| 12399 | 0 | m-D9 | H | tBu | A-38 | tBu | H |
| 12400 | 0 | m-D9 | H | tBu | A-39 | tBu | H |
| 12401 | 0 | m-D9 | H | tBu | A-40 | tBu | H |
| 12402 | 0 | m-D9 | H | tBu | A-41 | tBu | H |
| 12403 | 0 | m-D9 | H | tBu | A-42 | tBu | H |
| 12404 | 0 | m-D9 | H | tBu | A-43 | tBu | H |
| 12405 | 0 | m-D9 | H | tBu | A-44 | tBu | H |
| 12406 | 0 | m-D9 | H | tBu | A-45 | tBu | H |
| 12407 | 0 | m-D9 | H | tBu | A-46 | tBu | H |
| 12408 | 0 | m-D9 | H | tBu | A-47 | tBu | H |
| 12409 | 0 | m-D10 | H | tBu | A-1 | tBu | H |
| 12410 | 0 | m-D10 | H | tBu | A-2 | tBu | H |
| 12411 | 0 | m-D10 | H | tBu | A-3 | tBu | H |
| 12412 | 0 | m-D10 | H | tBu | A-4 | tBu | H |
| 12413 | 0 | m-D10 | H | tBu | A-5 | tBu | H |
| 12414 | 0 | m-D10 | H | tBu | A-6 | tBu | H |
| 12415 | 0 | m-D10 | H | tBu | A-7 | tBu | H |
| 12416 | 0 | m-D10 | H | tBu | A-8 | tBu | H |
| 12417 | 0 | m-D10 | H | tBu | A-9 | tBu | H |
| 12418 | 0 | m-D10 | H | tBu | A-10 | tBu | H |
| 12419 | 0 | m-D10 | H | tBu | A-11 | tBu | H |
| 12420 | 0 | m-D10 | H | tBu | A-12 | tBu | H |
| 12421 | 0 | m-D10 | H | tBu | A-13 | tBu | H |
| 12422 | 0 | m-D10 | H | tBu | A-14 | tBu | H |
| 12423 | 0 | m-D10 | H | tBu | A-15 | tBu | H |
| 12424 | 0 | m-D10 | H | tBu | A-16 | tBu | H |
| 12425 | 0 | m-D10 | H | tBu | A-17 | tBu | H |
| 12426 | 0 | m-D10 | H | tBu | A-18 | tBu | H |
| 12427 | 0 | m-D10 | H | tBu | A-19 | tBu | H |
| 12428 | 0 | m-D10 | H | tBu | A-20 | tBu | H |
| 12429 | 0 | m-D10 | H | tBu | A-21 | tBu | H |
| 12430 | 0 | m-D10 | H | tBu | A-22 | tBu | H |
| 12431 | 0 | m-D10 | H | tBu | A-23 | tBu | H |
| 12432 | 0 | m-D10 | H | tBu | A-24 | tBu | H |
| 12433 | 0 | m-D10 | H | tBu | A-25 | tBu | H |
| 12434 | 0 | m-D10 | H | tBu | A-26 | tBu | H |
| 12435 | 0 | m-D10 | H | tBu | A-27 | tBu | H |
| 12436 | 0 | m-D10 | H | tBu | A-28 | tBu | H |
| 12437 | 0 | m-D10 | H | tBu | A-29 | tBu | H |
| 12438 | 0 | m-D10 | H | tBu | A-30 | tBu | H |
| 12439 | 0 | m-D10 | H | tBu | A-31 | tBu | H |
| 12440 | 0 | m-D10 | H | tBu | A-32 | tBu | H |
| 12441 | 0 | m-D10 | H | tBu | A-33 | tBu | H |
| 12442 | 0 | m-D10 | H | tBu | A-34 | tBu | H |
| 12443 | 0 | m-D10 | H | tBu | A-35 | tBu | H |
| 12444 | 0 | m-D10 | H | tBu | A-36 | tBu | H |
| 12445 | 0 | m-D10 | H | tBu | A-37 | tBu | H |
| 12446 | 0 | m-D10 | H | tBu | A-38 | tBu | H |
| 12447 | 0 | m-D10 | H | tBu | A-39 | tBu | H |
| 12448 | 0 | m-D10 | H | tBu | A-40 | tBu | H |
| 12449 | 0 | m-D10 | H | tBu | A-41 | tBu | H |
| 12450 | 0 | m-D10 | H | tBu | A-42 | tBu | H |
| 12451 | 0 | m-D10 | H | tBu | A-43 | tBu | H |
| 12452 | 0 | m-D10 | H | tBu | A-44 | tBu | H |
| 12453 | 0 | m-D10 | H | tBu | A-45 | tBu | H |
| 12454 | 0 | m-D10 | H | tBu | A-46 | tBu | H |
| 12455 | 0 | m-D10 | H | tBu | A-47 | tBu | H |
| 12456 | 0 | m-D11 | H | tBu | A-1 | tBu | H |
| 12457 | 0 | m-D11 | H | tBu | A-2 | tBu | H |
| 12458 | 0 | m-D11 | H | tBu | A-3 | tBu | H |
| 12459 | 0 | m-D11 | H | tBu | A-4 | tBu | H |
| 12460 | 0 | m-D11 | H | tBu | A-5 | tBu | H |
| 12461 | 0 | m-D11 | H | tBu | A-6 | tBu | H |
| 12462 | 0 | m-D11 | H | tBu | A-7 | tBu | H |
| 12463 | 0 | m-D11 | H | tBu | A-8 | tBu | H |
| 12464 | 0 | m-D11 | H | tBu | A-9 | tBu | H |
| 12465 | 0 | m-D11 | H | tBu | A-10 | tBu | H |
| 12466 | 0 | m-D11 | H | tBu | A-11 | tBu | H |
| 12467 | 0 | m-D11 | H | tBu | A-12 | tBu | H |
| 12468 | 0 | m-D11 | H | tBu | A-13 | tBu | H |
| 12469 | 0 | m-D11 | H | tBu | A-14 | tBu | H |
| 12470 | 0 | m-D11 | H | tBu | A-15 | tBu | H |
| 12471 | 0 | m-D11 | H | tBu | A-16 | tBu | H |
| 12472 | 0 | m-D11 | H | tBu | A-17 | tBu | H |
| 12473 | 0 | m-D11 | H | tBu | A-18 | tBu | H |
| 12474 | 0 | m-D11 | H | tBu | A-19 | tBu | H |
| 12475 | 0 | m-D11 | H | tBu | A-20 | tBu | H |
| 12476 | 0 | m-D11 | H | tBu | A-21 | tBu | H |
| 12477 | 0 | m-D11 | H | tBu | A-22 | tBu | H |
| 12478 | 0 | m-D11 | H | tBu | A-23 | tBu | H |
| 12479 | 0 | m-D11 | H | tBu | A-24 | tBu | H |
| 12480 | 0 | m-D11 | H | tBu | A-25 | tBu | H |
| 12481 | 0 | m-D11 | H | tBu | A-26 | tBu | H |
| 12482 | 0 | m-D11 | H | tBu | A-27 | tBu | H |
| 12483 | 0 | m-D11 | H | tBu | A-28 | tBu | H |
| 12484 | 0 | m-D11 | H | tBu | A-29 | tBu | H |
| 12485 | 0 | m-D11 | H | tBu | A-30 | tBu | H |
| 12486 | 0 | m-D11 | H | tBu | A-31 | tBu | H |
| 12487 | 0 | m-D11 | H | tBu | A-32 | tBu | H |
| 12488 | 0 | m-D11 | H | tBu | A-33 | tBu | H |
| 12489 | 0 | m-D11 | H | tBu | A-34 | tBu | H |
| 12490 | 0 | m-D11 | H | tBu | A-35 | tBu | H |
| 12491 | 0 | m-D11 | H | tBu | A-36 | tBu | H |
| 12492 | 0 | m-D11 | H | tBu | A-37 | tBu | H |
| 12493 | 0 | m-D11 | H | tBu | A-38 | tBu | H |
| 12494 | 0 | m-D11 | H | tBu | A-39 | tBu | H |
| 12495 | 0 | m-D11 | H | tBu | A-40 | tBu | H |
| 12496 | 0 | m-D11 | H | tBu | A-41 | tBu | H |
| 12497 | 0 | m-D11 | H | tBu | A-42 | tBu | H |
| 12498 | 0 | m-D11 | H | tBu | A-43 | tBu | H |
| 12499 | 0 | m-D11 | H | tBu | A-44 | tBu | H |
| 12500 | 0 | m-D11 | H | tBu | A-45 | tBu | H |
| 12501 | 0 | m-D11 | H | tBu | A-46 | tBu | H |
| 12502 | 0 | m-D11 | H | tBu | A-47 | tBu | H |
| 12503 | 0 | m-D12 | H | tBu | A-1 | tBu | H |
| 12504 | 0 | m-D12 | H | tBu | A-2 | tBu | H |
| 12505 | 0 | m-D12 | H | tBu | A-3 | tBu | H |
| 12506 | 0 | m-D12 | H | tBu | A-4 | tBu | H |
| 12507 | 0 | m-D12 | H | tBu | A-5 | tBu | H |
| 12508 | 0 | m-D12 | H | tBu | A-6 | tBu | H |
| 12509 | 0 | m-D12 | H | tBu | A-7 | tBu | H |
| 12510 | 0 | m-D12 | H | tBu | A-8 | tBu | H |
| 12511 | 0 | m-D12 | H | tBu | A-9 | tBu | H |
| 12512 | 0 | m-D12 | H | tBu | A-10 | tBu | H |
| 12513 | 0 | m-D12 | H | tBu | A-11 | tBu | H |
| 12514 | 0 | m-D12 | H | tBu | A-12 | tBu | H |
| 12515 | 0 | m-D12 | H | tBu | A-13 | tBu | H |
| 12516 | 0 | m-D12 | H | tBu | A-14 | tBu | H |
| 12517 | 0 | m-D12 | H | tBu | A-15 | tBu | H |
| 12518 | 0 | m-D12 | H | tBu | A-16 | tBu | H |
| 12519 | 0 | m-D12 | H | tBu | A-17 | tBu | H |
| 12520 | 0 | m-D12 | H | tBu | A-18 | tBu | H |
| 12521 | 0 | m-D12 | H | tBu | A-19 | tBu | H |
| 12522 | 0 | m-D12 | H | tBu | A-20 | tBu | H |
| 12523 | 0 | m-D12 | H | tBu | A-21 | tBu | H |
| 12524 | 0 | m-D12 | H | tBu | A-22 | tBu | H |
| 12525 | 0 | m-D12 | H | tBu | A-23 | tBu | H |
| 12526 | 0 | m-D12 | H | tBu | A-24 | tBu | H |
| 12527 | 0 | m-D12 | H | tBu | A-25 | tBu | H |
| 12528 | 0 | m-D12 | H | tBu | A-26 | tBu | H |
| 12529 | 0 | m-D12 | H | tBu | A-27 | tBu | H |
| 12530 | 0 | m-D12 | H | tBu | A-28 | tBu | H |
| 12531 | 0 | m-D12 | H | tBu | A-29 | tBu | H |
| 12532 | 0 | m-D12 | H | tBu | A-30 | tBu | H |
| 12533 | 0 | m-D12 | H | tBu | A-31 | tBu | H |
| 12534 | 0 | m-D12 | H | tBu | A-32 | tBu | H |
| 12535 | 0 | m-D12 | H | tBu | A-33 | tBu | H |
| 12536 | 0 | m-D12 | H | tBu | A-34 | tBu | H |
| 12537 | 0 | m-D12 | H | tBu | A-35 | tBu | H |
| 12538 | 0 | m-D12 | H | tBu | A-36 | tBu | H |
| 12539 | 0 | m-D12 | H | tBu | A-37 | tBu | H |
| 12540 | 0 | m-D12 | H | tBu | A-38 | tBu | H |
| 12541 | 0 | m-D12 | H | tBu | A-39 | tBu | H |
| 12542 | 0 | m-D12 | H | tBu | A-40 | tBu | H |
| 12543 | 0 | m-D12 | H | tBu | A-41 | tBu | H |
| 12544 | 0 | m-D12 | H | tBu | A-42 | tBu | H |
| 12545 | 0 | m-D12 | H | tBu | A-43 | tBu | H |
| 12546 | 0 | m-D12 | H | tBu | A-44 | tBu | H |
| 12547 | 0 | m-D12 | H | tBu | A-45 | tBu | H |
| 12548 | 0 | m-D12 | H | tBu | A-46 | tBu | H |
| 12549 | 0 | m-D12 | H | tBu | A-47 | tBu | H |
| 12550 | 0 | m-D13 | H | tBu | A-1 | tBu | H |
| 12551 | 0 | m-D13 | H | tBu | A-2 | tBu | H |
| 12552 | 0 | m-D13 | H | tBu | A-3 | tBu | H |
| 12553 | 0 | m-D13 | H | tBu | A-4 | tBu | H |
| 12554 | 0 | m-D13 | H | tBu | A-5 | tBu | H |
| 12555 | 0 | m-D13 | H | tBu | A-6 | tBu | H |
| 12556 | 0 | m-D13 | H | tBu | A-7 | tBu | H |
| 12557 | 0 | m-D13 | H | tBu | A-8 | tBu | H |
| 12558 | 0 | m-D13 | H | tBu | A-9 | tBu | H |
| 12559 | 0 | m-D13 | H | tBu | A-10 | tBu | H |
| 12560 | 0 | m-D13 | H | tBu | A-11 | tBu | H |
| 12561 | 0 | m-D13 | H | tBu | A-12 | tBu | H |
| 12562 | 0 | m-D13 | H | tBu | A-13 | tBu | H |
| 12563 | 0 | m-D13 | H | tBu | A-14 | tBu | H |
| 12564 | 0 | m-D13 | H | tBu | A-15 | tBu | H |
| 12565 | 0 | m-D13 | H | tBu | A-16 | tBu | H |
| 12566 | 0 | m-D13 | H | tBu | A-17 | tBu | H |
| 12567 | 0 | m-D13 | H | tBu | A-18 | tBu | H |
| 12568 | 0 | m-D13 | H | tBu | A-19 | tBu | H |
| 12569 | 0 | m-D13 | H | tBu | A-20 | tBu | H |
| 12570 | 0 | m-D13 | H | tBu | A-21 | tBu | H |
| 12571 | 0 | m-D13 | H | tBu | A-22 | tBu | H |
| 12572 | 0 | m-D13 | H | tBu | A-23 | tBu | H |
| 12573 | 0 | m-D13 | H | tBu | A-24 | tBu | H |
| 12574 | 0 | m-D13 | H | tBu | A-25 | tBu | H |
| 12575 | 0 | m-D13 | H | tBu | A-26 | tBu | H |
| 12576 | 0 | m-D13 | H | tBu | A-27 | tBu | H |
| 12577 | 0 | m-D13 | H | tBu | A-28 | tBu | H |
| 12578 | 0 | m-D13 | H | tBu | A-29 | tBu | H |
| 12579 | 0 | m-D13 | H | tBu | A-30 | tBu | H |
| 12580 | 0 | m-D13 | H | tBu | A-31 | tBu | H |
| 12581 | 0 | m-D13 | H | tBu | A-32 | tBu | H |
| 12582 | 0 | m-D13 | H | tBu | A-33 | tBu | H |
| 12583 | 0 | m-D13 | H | tBu | A-34 | tBu | H |
| 12584 | 0 | m-D13 | H | tBu | A-35 | tBu | H |
| 12585 | 0 | m-D13 | H | tBu | A-36 | tBu | H |
| 12586 | 0 | m-D13 | H | tBu | A-37 | tBu | H |
| 12587 | 0 | m-D13 | H | tBu | A-38 | tBu | H |
| 12588 | 0 | m-D13 | H | tBu | A-39 | tBu | H |
| 12589 | 0 | m-D13 | H | tBu | A-40 | tBu | H |
| 12590 | 0 | m-D13 | H | tBu | A-41 | tBu | H |
| 12591 | 0 | m-D13 | H | tBu | A-42 | tBu | H |
| 12592 | 0 | m-D13 | H | tBu | A-43 | tBu | H |
| 12593 | 0 | m-D13 | H | tBu | A-44 | tBu | H |
| 12594 | 0 | m-D13 | H | tBu | A-45 | tBu | H |
| 12595 | 0 | m-D13 | H | tBu | A-46 | tBu | H |
| 12596 | 0 | m-D13 | H | tBu | A-47 | tBu | H |
| 12597 | 0 | m-D14 | H | tBu | A-1 | tBu | H |
| 12598 | 0 | m-D14 | H | tBu | A-2 | tBu | H |
| 12599 | 0 | m-D14 | H | tBu | A-3 | tBu | H |
| 12600 | 0 | m-D14 | H | tBu | A-4 | tBu | H |
| 12601 | 0 | m-D14 | H | tBu | A-5 | tBu | H |
| 12602 | 0 | m-D14 | H | tBu | A-6 | tBu | H |
| 12603 | 0 | m-D14 | H | tBu | A-7 | tBu | H |
| 12604 | 0 | m-D14 | H | tBu | A-8 | tBu | H |
| 12605 | 0 | m-D14 | H | tBu | A-9 | tBu | H |
| 12606 | 0 | m-D14 | H | tBu | A-10 | tBu | H |
| 12607 | 0 | m-D14 | H | tBu | A-11 | tBu | H |
| 12608 | 0 | m-D14 | H | tBu | A-12 | tBu | H |
| 12609 | 0 | m-D14 | H | tBu | A-13 | tBu | H |
| 12610 | 0 | m-D14 | H | tBu | A-14 | tBu | H |
| 12611 | 0 | m-D14 | H | tBu | A-15 | tBu | H |
| 12612 | 0 | m-D14 | H | tBu | A-16 | tBu | H |
| 12613 | 0 | m-D14 | H | tBu | A-17 | tBu | H |
| 12614 | 0 | m-D14 | H | tBu | A-18 | tBu | H |
| 12615 | 0 | m-D14 | H | tBu | A-19 | tBu | H |
| 12616 | 0 | m-D14 | H | tBu | A-20 | tBu | H |
| 12617 | 0 | m-D14 | H | tBu | A-21 | tBu | H |
| 12618 | 0 | m-D14 | H | tBu | A-22 | tBu | H |
| 12619 | 0 | m-D14 | H | tBu | A-23 | tBu | H |
| 12620 | 0 | m-D14 | H | tBu | A-24 | tBu | H |
| 12621 | 0 | m-D14 | H | tBu | A-25 | tBu | H |
| 12622 | 0 | m-D14 | H | tBu | A-26 | tBu | H |
| 12623 | 0 | m-D14 | H | tBu | A-27 | tBu | H |
| 12624 | 0 | m-D14 | H | tBu | A-28 | tBu | H |
| 12625 | 0 | m-D14 | H | tBu | A-29 | tBu | H |
| 12626 | 0 | m-D14 | H | tBu | A-30 | tBu | H |
| 12627 | 0 | m-D14 | H | tBu | A-31 | tBu | H |
| 12628 | 0 | m-D14 | H | tBu | A-32 | tBu | H |
| 12629 | 0 | m-D14 | H | tBu | A-33 | tBu | H |
| 12630 | 0 | m-D14 | H | tBu | A-34 | tBu | H |
| 12631 | 0 | m-D14 | H | tBu | A-35 | tBu | H |
| 12632 | 0 | m-D14 | H | tBu | A-36 | tBu | H |
| 12633 | 0 | m-D14 | H | tBu | A-37 | tBu | H |
| 12634 | 0 | m-D14 | H | tBu | A-38 | tBu | H |
| 12635 | 0 | m-D14 | H | tBu | A-39 | tBu | H |
| 12636 | 0 | m-D14 | H | tBu | A-40 | tBu | H |
| 12637 | 0 | m-D14 | H | tBu | A-41 | tBu | H |
| 12638 | 0 | m-D14 | H | tBu | A-42 | tBu | H |
| 12639 | 0 | m-D14 | H | tBu | A-43 | tBu | H |
| 12640 | 0 | m-D14 | H | tBu | A-44 | tBu | H |
| 12641 | 0 | m-D14 | H | tBu | A-45 | tBu | H |
| 12642 | 0 | m-D14 | H | tBu | A-46 | tBu | H |
| 12643 | 0 | m-D14 | H | tBu | A-47 | tBu | H |

Lengthy table referenced here
US20240260455A1-20240801-T00001
Please refer to the end of the specification for access instructions.

TABLE 13-1

| No. | t | ×1 | ×2 | ×3 | ×4 | ×5 | ×6 | ×7 | ×8 | ×9 | ×10 |
|---|---|---|---|---|---|---|---|---|---|---|---|
| 38259 | 1 | H | H | m-D1 | H | H | H | H | A-1 | H | H |
| 38260 | 1 | H | H | m-D1 | H | H | H | H | A-2 | H | H |
| 38261 | 1 | H | H | m-D1 | H | H | H | H | A-3 | H | H |
| 38262 | 1 | H | H | m-D1 | H | H | H | H | A-4 | H | H |
| 38263 | 1 | H | H | m-D1 | H | H | H | H | A-5 | H | H |
| 38264 | 1 | H | H | m-D1 | H | H | H | H | A-6 | H | H |
| 38265 | 1 | H | H | m-D1 | H | H | H | H | A-7 | H | H |
| 38266 | 1 | H | H | m-D1 | H | H | H | H | A-8 | H | H |
| 38267 | 1 | H | H | m-D1 | H | H | H | H | A-9 | H | H |
| 38268 | 1 | H | H | m-D1 | H | H | H | H | A-10 | H | H |
| 38269 | 1 | H | H | m-D1 | H | H | H | H | A-11 | H | H |
| 38270 | 1 | H | H | m-D1 | H | H | H | H | A-12 | H | H |
| 38271 | 1 | H | H | m-D1 | H | H | H | H | A-13 | H | H |
| 38272 | 1 | H | H | m-D1 | H | H | H | H | A-14 | H | H |
| 38273 | 1 | H | H | m-D1 | H | H | H | H | A-15 | H | H |
| 38274 | 1 | H | H | m-D1 | H | H | H | H | A-16 | H | H |
| 38275 | 1 | H | H | m-D1 | H | H | H | H | A-17 | H | H |
| 38276 | 1 | H | H | m-D1 | H | H | H | H | A-18 | H | H |
| 38277 | 1 | H | H | m-D1 | H | H | H | H | A-19 | H | H |
| 38278 | 1 | H | H | m-D1 | H | H | H | H | A-20 | H | H |
| 38279 | 1 | H | H | m-D1 | H | H | H | H | A-21 | H | H |
| 38280 | 1 | H | H | m-D1 | H | H | H | H | A-22 | H | H |
| 38281 | 1 | H | H | m-D1 | H | H | H | H | A-23 | H | H |
| 38282 | 1 | H | H | m-D1 | H | H | H | H | A-24 | H | H |
| 38283 | 1 | H | H | m-D1 | H | H | H | H | A-25 | H | H |
| 38284 | 1 | H | H | m-D1 | H | H | H | H | A-26 | H | H |
| 38285 | 1 | H | H | m-D1 | H | H | H | H | A-27 | H | H |
| 38286 | 1 | H | H | m-D1 | H | H | H | H | A-28 | H | H |
| 38287 | 1 | H | H | m-D1 | H | H | H | H | A-29 | H | H |
| 38288 | 1 | H | H | m-D1 | H | H | H | H | A-30 | H | H |
| 38289 | 1 | H | H | m-D1 | H | H | H | H | A-31 | H | H |
| 38290 | 1 | H | H | m-D1 | H | H | H | H | A-32 | H | H |
| 38291 | 1 | H | H | m-D1 | H | H | H | H | A-33 | H | H |
| 38292 | 1 | H | H | m-D1 | H | H | H | H | A-34 | H | H |
| 38293 | 1 | H | H | m-D1 | H | H | H | H | A-35 | H | H |
| 38294 | 1 | H | H | m-D1 | H | H | H | H | A-36 | H | H |
| 38295 | 1 | H | H | m-D1 | H | H | H | H | A-37 | H | H |
| 38296 | 1 | H | H | m-D1 | H | H | H | H | A-38 | H | H |
| 38297 | 1 | H | H | m-D1 | H | H | H | H | A-39 | H | H |
| 38298 | 1 | H | H | m-D1 | H | H | H | H | A-40 | H | H |
| 38299 | 1 | H | H | m-D1 | H | H | H | H | A-41 | H | H |
| 38300 | 1 | H | H | m-D1 | H | H | H | H | A-42 | H | H |
| 38301 | 1 | H | H | m-D1 | H | H | H | H | A-43 | H | H |
| 38302 | 1 | H | H | m-D1 | H | H | H | H | A-44 | H | H |
| 38303 | 1 | H | H | m-D1 | H | H | H | H | A-45 | H | H |
| 38304 | 1 | H | H | m-D1 | H | H | H | H | A-46 | H | H |
| 38305 | 1 | H | H | m-D1 | H | H | H | H | A-47 | H | H |
| 38306 | 1 | H | H | m-D2 | H | H | H | H | A-1 | H | H |
| 38307 | 1 | H | H | m-D2 | H | H | H | H | A-2 | H | H |
| 38308 | 1 | H | H | m-D2 | H | H | H | H | A-3 | H | H |
| 38309 | 1 | H | H | m-D2 | H | H | H | H | A-4 | H | H |
| 38310 | 1 | H | H | m-D2 | H | H | H | H | A-5 | H | H |
| 38311 | 1 | H | H | m-D2 | H | H | H | H | A-6 | H | H |
| 38312 | 1 | H | H | m-D2 | H | H | H | H | A-7 | H | H |
| 38313 | 1 | H | H | m-D2 | H | H | H | H | A-8 | H | H |
| 38314 | 1 | H | H | m-D2 | H | H | H | H | A-9 | H | H |
| 38315 | 1 | H | H | m-D2 | H | H | H | H | A-10 | H | H |
| 38316 | 1 | H | H | m-D2 | H | H | H | H | A-11 | H | H |
| 38317 | 1 | H | H | m-D2 | H | H | H | H | A-12 | H | H |
| 38318 | 1 | H | H | m-D2 | H | H | H | H | A-13 | H | H |
| 38319 | 1 | H | H | m-D2 | H | H | H | H | A-14 | H | H |
| 38320 | 1 | H | H | m-D2 | H | H | H | H | A-15 | H | H |
| 38321 | 1 | H | H | m-D2 | H | H | H | H | A-16 | H | H |
| 38322 | 1 | H | H | m-D2 | H | H | H | H | A-17 | H | H |
| 38323 | 1 | H | H | m-D2 | H | H | H | H | A-18 | H | H |
| 38324 | 1 | H | H | m-D2 | H | H | H | H | A-19 | H | H |
| 38325 | 1 | H | H | m-D2 | H | H | H | H | A-20 | H | H |
| 38326 | 1 | H | H | m-D2 | H | H | H | H | A-21 | H | H |
| 38327 | 1 | H | H | m-D2 | H | H | H | H | A-22 | H | H |
| 38328 | 1 | H | H | m-D2 | H | H | H | H | A-23 | H | H |
| 38329 | 1 | H | H | m-D2 | H | H | H | H | A-24 | H | H |
| 38330 | 1 | H | H | m-D2 | H | H | H | H | A-25 | H | H |
| 38331 | 1 | H | H | m-D2 | H | H | H | H | A-26 | H | H |
| 38332 | 1 | H | H | m-D2 | H | H | H | H | A-27 | H | H |
| 38333 | 1 | H | H | m-D2 | H | H | H | H | A-28 | H | H |
| 38334 | 1 | H | H | m-D2 | H | H | H | H | A-29 | H | H |
| 38335 | 1 | H | H | m-D2 | H | H | H | H | A-30 | H | H |
| 38336 | 1 | H | H | m-D2 | H | H | H | H | A-31 | H | H |
| 38337 | 1 | H | H | m-D2 | H | H | H | H | A-32 | H | H |
| 38338 | 1 | H | H | m-D2 | H | H | H | H | A-33 | H | H |
| 38339 | 1 | H | H | m-D2 | H | H | H | H | A-34 | H | H |
| 38340 | 1 | H | H | m-D2 | H | H | H | H | A-35 | H | H |
| 38341 | 1 | H | H | m-D2 | H | H | H | H | A-36 | H | H |
| 38342 | 1 | H | H | m-D2 | H | H | H | H | A-37 | H | H |
| 38343 | 1 | H | H | m-D2 | H | H | H | H | A-38 | H | H |
| 38344 | 1 | H | H | m-D2 | H | H | H | H | A-39 | H | H |
| 38345 | 1 | H | H | m-D2 | H | H | H | H | A-40 | H | H |
| 38346 | 1 | H | H | m-D2 | H | H | H | H | A-41 | H | H |
| 38347 | 1 | H | H | m-D2 | H | H | H | H | A-42 | H | H |
| 38348 | 1 | H | H | m-D2 | H | H | H | H | A-43 | H | H |
| 38349 | 1 | H | H | m-D2 | H | H | H | H | A-44 | H | H |
| 38350 | 1 | H | H | m-D2 | H | H | H | H | A-45 | H | H |
| 38351 | 1 | H | H | m-D2 | H | H | H | H | A-46 | H | H |
| 38352 | 1 | H | H | m-D2 | H | H | H | H | A-47 | H | H |
| 38353 | 1 | H | H | m-D3 | H | H | H | H | A-1 | H | H |
| 38354 | 1 | H | H | m-D3 | H | H | H | H | A-2 | H | H |
| 38355 | 1 | H | H | m-D3 | H | H | H | H | A-3 | H | H |
| 38356 | 1 | H | H | m-D3 | H | H | H | H | A-4 | H | H |
| 38357 | 1 | H | H | m-D3 | H | H | H | H | A-5 | H | H |
| 38358 | 1 | H | H | m-D3 | H | H | H | H | A-6 | H | H |
| 38359 | 1 | H | H | m-D3 | H | H | H | H | A-7 | H | H |
| 38360 | 1 | H | H | m-D3 | H | H | H | H | A-8 | H | H |
| 38361 | 1 | H | H | m-D3 | H | H | H | H | A-9 | H | H |
| 38362 | 1 | H | H | m-D3 | H | H | H | H | A-10 | H | H |
| 38363 | 1 | H | H | m-D3 | H | H | H | H | A-11 | H | H |
| 38364 | 1 | H | H | m-D3 | H | H | H | H | A-12 | H | H |
| 38365 | 1 | H | H | m-D3 | H | H | H | H | A-13 | H | H |
| 38366 | 1 | H | H | m-D3 | H | H | H | H | A-14 | H | H |
| 38367 | 1 | H | H | m-D3 | H | H | H | H | A-15 | H | H |
| 38368 | 1 | H | H | m-D3 | H | H | H | H | A-16 | H | H |
| 38369 | 1 | H | H | m-D3 | H | H | H | H | A-17 | H | H |
| 38370 | 1 | H | H | m-D3 | H | H | H | H | A-18 | H | H |
| 38371 | 1 | H | H | m-D3 | H | H | H | H | A-19 | H | H |
| 38372 | 1 | H | H | m-D3 | H | H | H | H | A-20 | H | H |
| 38373 | 1 | H | H | m-D3 | H | H | H | H | A-21 | H | H |
| 38374 | 1 | H | H | m-D3 | H | H | H | H | A-22 | H | H |
| 38375 | 1 | H | H | m-D3 | H | H | H | H | A-23 | H | H |
| 38376 | 1 | H | H | m-D3 | H | H | H | H | A-24 | H | H |
| 38377 | 1 | H | H | m-D3 | H | H | H | H | A-25 | H | H |
| 38378 | 1 | H | H | m-D3 | H | H | H | H | A-26 | H | H |
| 38379 | 1 | H | H | m-D3 | H | H | H | H | A-27 | H | H |
| 38380 | 1 | H | H | m-D3 | H | H | H | H | A-28 | H | H |
| 38381 | 1 | H | H | m-D3 | H | H | H | H | A-29 | H | H |
| 38382 | 1 | H | H | m-D3 | H | H | H | H | A-30 | H | H |
| 38383 | 1 | H | H | m-D3 | H | H | H | H | A-31 | H | H |
| 38384 | 1 | H | H | m-D3 | H | H | H | H | A-32 | H | H |
| 38385 | 1 | H | H | m-D3 | H | H | H | H | A-33 | H | H |
| 38386 | 1 | H | H | m-D3 | H | H | H | H | A-34 | H | H |
| 38387 | 1 | H | H | m-D3 | H | H | H | H | A-35 | H | H |
| 38388 | 1 | H | H | m-D3 | H | H | H | H | A-36 | H | H |
| 38389 | 1 | H | H | m-D3 | H | H | H | H | A-37 | H | H |
| 38390 | 1 | H | H | m-D3 | H | H | H | H | A-38 | H | H |
| 38391 | 1 | H | H | m-D3 | H | H | H | H | A-39 | H | H |
| 38392 | 1 | H | H | m-D3 | H | H | H | H | A-40 | H | H |
| 38393 | 1 | H | H | m-D3 | H | H | H | H | A-41 | H | H |
| 38394 | 1 | H | H | m-D3 | H | H | H | H | A-42 | H | H |
| 38395 | 1 | H | H | m-D3 | H | H | H | H | A-43 | H | H |
| 38396 | 1 | H | H | m-D3 | H | H | H | H | A-44 | H | H |
| 38397 | 1 | H | H | m-D3 | H | H | H | H | A-45 | H | H |
| 38398 | 1 | H | H | m-D3 | H | H | H | H | A-46 | H | H |
| 38399 | 1 | H | H | m-D3 | H | H | H | H | A-47 | H | H |
| 38400 | 1 | H | H | m-D4 | H | H | H | H | A-1 | H | H |
| 38401 | 1 | H | H | m-D4 | H | H | H | H | A-2 | H | H |
| 38402 | 1 | H | H | m-D4 | H | H | H | H | A-3 | H | H |
| 38403 | 1 | H | H | m-D4 | H | H | H | H | A-4 | H | H |
| 38404 | 1 | H | H | m-D4 | H | H | H | H | A-5 | H | H |
| 38405 | 1 | H | H | m-D4 | H | H | H | H | A-6 | H | H |
| 38406 | 1 | H | H | m-D4 | H | H | H | H | A-7 | H | H |
| 38407 | 1 | H | H | m-D4 | H | H | H | H | A-8 | H | H |
| 38408 | 1 | H | H | m-D4 | H | H | H | H | A-9 | H | H |
| 38409 | 1 | H | H | m-D4 | H | H | H | H | A-10 | H | H |
| 38410 | 1 | H | H | m-D4 | H | H | H | H | A-11 | H | H |
| 38411 | 1 | H | H | m-D4 | H | H | H | H | A-12 | H | H |
| 38412 | 1 | H | H | m-D4 | H | H | H | H | A-13 | H | H |
| 38413 | 1 | H | H | m-D4 | H | H | H | H | A-14 | H | H |
| 38414 | 1 | H | H | m-D4 | H | H | H | H | A-15 | H | H |
| 38415 | 1 | H | H | m-D4 | H | H | H | H | A-16 | H | H |
| 38416 | 1 | H | H | m-D4 | H | H | H | H | A-17 | H | H |
| 38417 | 1 | H | H | m-D4 | H | H | H | H | A-18 | H | H |
| 38418 | 1 | H | H | m-D4 | H | H | H | H | A-19 | H | H |
| 38419 | 1 | H | H | m-D4 | H | H | H | H | A-20 | H | H |
| 38420 | 1 | H | H | m-D4 | H | H | H | H | A-21 | H | H |
| 38421 | 1 | H | H | m-D4 | H | H | H | H | A-22 | H | H |
| 38422 | 1 | H | H | m-D4 | H | H | H | H | A-23 | H | H |
| 38423 | 1 | H | H | m-D4 | H | H | H | H | A-24 | H | H |
| 38424 | 1 | H | H | m-D4 | H | H | H | H | A-25 | H | H |
| 38425 | 1 | H | H | m-D4 | H | H | H | H | A-26 | H | H |
| 38426 | 1 | H | H | m-D4 | H | H | H | H | A-27 | H | H |
| 38427 | 1 | H | H | m-D4 | H | H | H | H | A-28 | H | H |
| 38428 | 1 | H | H | m-D4 | H | H | H | H | A-29 | H | H |
| 38429 | 1 | H | H | m-D4 | H | H | H | H | A-30 | H | H |
| 38430 | 1 | H | H | m-D4 | H | H | H | H | A-31 | H | H |
| 38431 | 1 | H | H | m-D4 | H | H | H | H | A-32 | H | H |
| 38432 | 1 | H | H | m-D4 | H | H | H | H | A-33 | H | H |
| 38433 | 1 | H | H | m-D4 | H | H | H | H | A-34 | H | H |
| 38434 | 1 | H | H | m-D4 | H | H | H | H | A-35 | H | H |
| 38435 | 1 | H | H | m-D4 | H | H | H | H | A-36 | H | H |
| 38436 | 1 | H | H | m-D4 | H | H | H | H | A-37 | H | H |
| 38437 | 1 | H | H | m-D4 | H | H | H | H | A-38 | H | H |
| 38438 | 1 | H | H | m-D4 | H | H | H | H | A-39 | H | H |
| 38439 | 1 | H | H | m-D4 | H | H | H | H | A-40 | H | H |
| 38440 | 1 | H | H | m-D4 | H | H | H | H | A-41 | H | H |
| 38441 | 1 | H | H | m-D4 | H | H | H | H | A-42 | H | H |
| 38442 | 1 | H | H | m-D4 | H | H | H | H | A-43 | H | H |
| 38443 | 1 | H | H | m-D4 | H | H | H | H | A-44 | H | H |
| 38444 | 1 | H | H | m-D4 | H | H | H | H | A-45 | H | H |
| 38445 | 1 | H | H | m-D4 | H | H | H | H | A-46 | H | H |
| 38446 | 1 | H | H | m-D4 | H | H | H | H | A-47 | H | H |
| 38447 | 1 | H | H | m-D5 | H | H | H | H | A-1 | H | H |
| 38448 | 1 | H | H | m-D5 | H | H | H | H | A-2 | H | H |
| 38449 | 1 | H | H | m-D5 | H | H | H | H | A-3 | H | H |
| 38450 | 1 | H | H | m-D5 | H | H | H | H | A-4 | H | H |
| 38451 | 1 | H | H | m-D5 | H | H | H | H | A-5 | H | H |
| 38452 | 1 | H | H | m-D5 | H | H | H | H | A-6 | H | H |
| 38453 | 1 | H | H | m-D5 | H | H | H | H | A-7 | H | H |
| 38454 | 1 | H | H | m-D5 | H | H | H | H | A-8 | H | H |
| 38455 | 1 | H | H | m-D5 | H | H | H | H | A-9 | H | H |
| 38456 | 1 | H | H | m-D5 | H | H | H | H | A-10 | H | H |
| 38457 | 1 | H | H | m-D5 | H | H | H | H | A-11 | H | H |
| 38458 | 1 | H | H | m-D5 | H | H | H | H | A-12 | H | H |
| 38459 | 1 | H | H | m-D5 | H | H | H | H | A-13 | H | H |
| 38460 | 1 | H | H | m-D5 | H | H | H | H | A-14 | H | H |
| 38461 | 1 | H | H | m-D5 | H | H | H | H | A-15 | H | H |
| 38462 | 1 | H | H | m-D5 | H | H | H | H | A-16 | H | H |
| 38463 | 1 | H | H | m-D5 | H | H | H | H | A-17 | H | H |
| 38464 | 1 | H | H | m-D5 | H | H | H | H | A-18 | H | H |
| 38465 | 1 | H | H | m-D5 | H | H | H | H | A-19 | H | H |
| 38466 | 1 | H | H | m-D5 | H | H | H | H | A-20 | H | H |
| 38467 | 1 | H | H | m-D5 | H | H | H | H | A-21 | H | H |
| 38468 | 1 | H | H | m-D5 | H | H | H | H | A-22 | H | H |
| 38469 | 1 | H | H | m-D5 | H | H | H | H | A-23 | H | H |
| 38470 | 1 | H | H | m-D5 | H | H | H | H | A-24 | H | H |
| 38471 | 1 | H | H | m-D5 | H | H | H | H | A-25 | H | H |
| 38472 | 1 | H | H | m-D5 | H | H | H | H | A-26 | H | H |
| 38473 | 1 | H | H | m-D5 | H | H | H | H | A-27 | H | H |
| 38474 | 1 | H | H | m-D5 | H | H | H | H | A-28 | H | H |
| 38475 | 1 | H | H | m-D5 | H | H | H | H | A-29 | H | H |
| 38476 | 1 | H | H | m-D5 | H | H | H | H | A-30 | H | H |
| 38477 | 1 | H | H | m-D5 | H | H | H | H | A-31 | H | H |
| 38478 | 1 | H | H | m-D5 | H | H | H | H | A-32 | H | H |
| 38479 | 1 | H | H | m-D5 | H | H | H | H | A-33 | H | H |
| 38480 | 1 | H | H | m-D5 | H | H | H | H | A-34 | H | H |
| 38481 | 1 | H | H | m-D5 | H | H | H | H | A-35 | H | H |
| 38482 | 1 | H | H | m-D5 | H | H | H | H | A-36 | H | H |
| 38483 | 1 | H | H | m-D5 | H | H | H | H | A-37 | H | H |
| 38484 | 1 | H | H | m-D5 | H | H | H | H | A-38 | H | H |
| 38485 | 1 | H | H | m-D5 | H | H | H | H | A-39 | H | H |
| 38486 | 1 | H | H | m-D5 | H | H | H | H | A-40 | H | H |
| 38487 | 1 | H | H | m-D5 | H | H | H | H | A-41 | H | H |
| 38488 | 1 | H | H | m-D5 | H | H | H | H | A-42 | H | H |
| 38489 | 1 | H | H | m-D5 | H | H | H | H | A-43 | H | H |
| 38490 | 1 | H | H | m-D5 | H | H | H | H | A-44 | H | H |
| 38491 | 1 | H | H | m-D5 | H | H | H | H | A-45 | H | H |
| 38492 | 1 | H | H | m-D5 | H | H | H | H | A-46 | H | H |
| 38493 | 1 | H | H | m-D5 | H | H | H | H | A-47 | H | H |
| 38494 | 1 | H | H | m-D6 | H | H | H | H | A-1 | H | H |
| 38495 | 1 | H | H | m-D6 | H | H | H | H | A-2 | H | H |
| 38496 | 1 | H | H | m-D6 | H | H | H | H | A-3 | H | H |
| 38497 | 1 | H | H | m-D6 | H | H | H | H | A-4 | H | H |
| 38498 | 1 | H | H | m-D6 | H | H | H | H | A-5 | H | H |
| 38499 | 1 | H | H | m-D6 | H | H | H | H | A-6 | H | H |
| 38500 | 1 | H | H | m-D6 | H | H | H | H | A-7 | H | H |
| 38501 | 1 | H | H | m-D6 | H | H | H | H | A-8 | H | H |
| 38502 | 1 | H | H | m-D6 | H | H | H | H | A-9 | H | H |
| 38503 | 1 | H | H | m-D6 | H | H | H | H | A-10 | H | H |
| 38504 | 1 | H | H | m-D6 | H | H | H | H | A-11 | H | H |
| 38505 | 1 | H | H | m-D6 | H | H | H | H | A-12 | H | H |
| 38506 | 1 | H | H | m-D6 | H | H | H | H | A-13 | H | H |
| 38507 | 1 | H | H | m-D6 | H | H | H | H | A-14 | H | H |
| 38508 | 1 | H | H | m-D6 | H | H | H | H | A-15 | H | H |
| 38509 | 1 | H | H | m-D6 | H | H | H | H | A-16 | H | H |
| 38510 | 1 | H | H | m-D6 | H | H | H | H | A-17 | H | H |
| 38511 | 1 | H | H | m-D6 | H | H | H | H | A-18 | H | H |
| 38512 | 1 | H | H | m-D6 | H | H | H | H | A-19 | H | H |
| 38513 | 1 | H | H | m-D6 | H | H | H | H | A-20 | H | H |
| 38514 | 1 | H | H | m-D6 | H | H | H | H | A-21 | H | H |
| 38515 | 1 | H | H | m-D6 | H | H | H | H | A-22 | H | H |
| 38516 | 1 | H | H | m-D6 | H | H | H | H | A-23 | H | H |
| 38517 | 1 | H | H | m-D6 | H | H | H | H | A-24 | H | H |
| 38518 | 1 | H | H | m-D6 | H | H | H | H | A-25 | H | H |
| 38519 | 1 | H | H | m-D6 | H | H | H | H | A-26 | H | H |
| 38520 | 1 | H | H | m-D6 | H | H | H | H | A-27 | H | H |
| 38521 | 1 | H | H | m-D6 | H | H | H | H | A-28 | H | H |
| 38522 | 1 | H | H | m-D6 | H | H | H | H | A-29 | H | H |
| 38523 | 1 | H | H | m-D6 | H | H | H | H | A-30 | H | H |
| 38524 | 1 | H | H | m-D6 | H | H | H | H | A-31 | H | H |
| 38525 | 1 | H | H | m-D6 | H | H | H | H | A-32 | H | H |
| 38526 | 1 | H | H | m-D6 | H | H | H | H | A-33 | H | H |
| 38527 | 1 | H | H | m-D6 | H | H | H | H | A-34 | H | H |
| 38528 | 1 | H | H | m-D6 | H | H | H | H | A-35 | H | H |
| 38529 | 1 | H | H | m-D6 | H | H | H | H | A-36 | H | H |
| 38530 | 1 | H | H | m-D6 | H | H | H | H | A-37 | H | H |
| 38531 | 1 | H | H | m-D6 | H | H | H | H | A-38 | H | H |
| 38532 | 1 | H | H | m-D6 | H | H | H | H | A-39 | H | H |
| 38533 | 1 | H | H | m-D6 | H | H | H | H | A-40 | H | H |
| 38534 | 1 | H | H | m-D6 | H | H | H | H | A-41 | H | H |
| 38535 | 1 | H | H | m-D6 | H | H | H | H | A-42 | H | H |
| 38536 | 1 | H | H | m-D6 | H | H | H | H | A-43 | H | H |
| 38537 | 1 | H | H | m-D6 | H | H | H | H | A-44 | H | H |
| 38538 | 1 | H | H | m-D6 | H | H | H | H | A-45 | H | H |
| 38539 | 1 | H | H | m-D6 | H | H | H | H | A-46 | H | H |
| 38540 | 1 | H | H | m-D6 | H | H | H | H | A-47 | H | H |
| 38541 | 1 | H | H | m-D7 | H | H | H | H | A-1 | H | H |
| 38542 | 1 | H | H | m-D7 | H | H | H | H | A-2 | H | H |
| 38543 | 1 | H | H | m-D7 | H | H | H | H | A-3 | H | H |
| 38544 | 1 | H | H | m-D7 | H | H | H | H | A-4 | H | H |
| 38545 | 1 | H | H | m-D7 | H | H | H | H | A-5 | H | H |
| 38546 | 1 | H | H | m-D7 | H | H | H | H | A-6 | H | H |
| 38547 | 1 | H | H | m-D7 | H | H | H | H | A-7 | H | H |
| 38548 | 1 | H | H | m-D7 | H | H | H | H | A-8 | H | H |
| 38549 | 1 | H | H | m-D7 | H | H | H | H | A-9 | H | H |
| 38550 | 1 | H | H | m-D7 | H | H | H | H | A-10 | H | H |
| 38551 | 1 | H | H | m-D7 | H | H | H | H | A-11 | H | H |
| 38552 | 1 | H | H | m-D7 | H | H | H | H | A-12 | H | H |
| 38553 | 1 | H | H | m-D7 | H | H | H | H | A-13 | H | H |
| 38554 | 1 | H | H | m-D7 | H | H | H | H | A-14 | H | H |
| 38555 | 1 | H | H | m-D7 | H | H | H | H | A-15 | H | H |
| 38556 | 1 | H | H | m-D7 | H | H | H | H | A-16 | H | H |
| 38557 | 1 | H | H | m-D7 | H | H | H | H | A-17 | H | H |
| 38558 | 1 | H | H | m-D7 | H | H | H | H | A-18 | H | H |
| 38559 | 1 | H | H | m-D7 | H | H | H | H | A-19 | H | H |
| 38560 | 1 | H | H | m-D7 | H | H | H | H | A-20 | H | H |
| 38561 | 1 | H | H | m-D7 | H | H | H | H | A-21 | H | H |
| 38562 | 1 | H | H | m-D7 | H | H | H | H | A-22 | H | H |
| 38563 | 1 | H | H | m-D7 | H | H | H | H | A-23 | H | H |
| 38564 | 1 | H | H | m-D7 | H | H | H | H | A-24 | H | H |
| 38565 | 1 | H | H | m-D7 | H | H | H | H | A-25 | H | H |
| 38566 | 1 | H | H | m-D7 | H | H | H | H | A-26 | H | H |
| 38567 | 1 | H | H | m-D7 | H | H | H | H | A-27 | H | H |
| 38568 | 1 | H | H | m-D7 | H | H | H | H | A-28 | H | H |
| 38569 | 1 | H | H | m-D7 | H | H | H | H | A-29 | H | H |
| 38570 | 1 | H | H | m-D7 | H | H | H | H | A-30 | H | H |
| 38571 | 1 | H | H | m-D7 | H | H | H | H | A-31 | H | H |
| 38572 | 1 | H | H | m-D7 | H | H | H | H | A-32 | H | H |
| 38573 | 1 | H | H | m-D7 | H | H | H | H | A-33 | H | H |
| 38574 | 1 | H | H | m-D7 | H | H | H | H | A-34 | H | H |
| 38575 | 1 | H | H | m-D7 | H | H | H | H | A-35 | H | H |
| 38576 | 1 | H | H | m-D7 | H | H | H | H | A-36 | H | H |
| 38577 | 1 | H | H | m-D7 | H | H | H | H | A-37 | H | H |
| 38578 | 1 | H | H | m-D7 | H | H | H | H | A-38 | H | H |
| 38579 | 1 | H | H | m-D7 | H | H | H | H | A-39 | H | H |
| 38580 | 1 | H | H | m-D7 | H | H | H | H | A-40 | H | H |
| 38581 | 1 | H | H | m-D7 | H | H | H | H | A-41 | H | H |
| 38582 | 1 | H | H | m-D7 | H | H | H | H | A-42 | H | H |
| 38583 | 1 | H | H | m-D7 | H | H | H | H | A-43 | H | H |
| 38584 | 1 | H | H | m-D7 | H | H | H | H | A-44 | H | H |
| 38585 | 1 | H | H | m-D7 | H | H | H | H | A-45 | H | H |
| 38586 | 1 | H | H | m-D7 | H | H | H | H | A-46 | H | H |
| 38587 | 1 | H | H | m-D7 | H | H | H | H | A-47 | H | H |
| 38588 | 1 | H | H | m-D8 | H | H | H | H | A-1 | H | H |
| 38589 | 1 | H | H | m-D8 | H | H | H | H | A-2 | H | H |
| 38590 | 1 | H | H | m-D8 | H | H | H | H | A-3 | H | H |
| 38591 | 1 | H | H | m-D8 | H | H | H | H | A-4 | H | H |
| 38592 | 1 | H | H | m-D8 | H | H | H | H | A-5 | H | H |
| 38593 | 1 | H | H | m-D8 | H | H | H | H | A-6 | H | H |
| 38594 | 1 | H | H | m-D8 | H | H | H | H | A-7 | H | H |
| 38595 | 1 | H | H | m-D8 | H | H | H | H | A-8 | H | H |
| 38596 | 1 | H | H | m-D8 | H | H | H | H | A-9 | H | H |
| 38597 | 1 | H | H | m-D8 | H | H | H | H | A-10 | H | H |
| 38598 | 1 | H | H | m-D8 | H | H | H | H | A-11 | H | H |
| 38599 | 1 | H | H | m-D8 | H | H | H | H | A-12 | H | H |
| 38600 | 1 | H | H | m-D8 | H | H | H | H | A-13 | H | H |
| 38601 | 1 | H | H | m-D8 | H | H | H | H | A-14 | H | H |
| 38602 | 1 | H | H | m-D8 | H | H | H | H | A-15 | H | H |
| 38603 | 1 | H | H | m-D8 | H | H | H | H | A-16 | H | H |
| 38604 | 1 | H | H | m-D8 | H | H | H | H | A-17 | H | H |
| 38605 | 1 | H | H | m-D8 | H | H | H | H | A-18 | H | H |
| 38606 | 1 | H | H | m-D8 | H | H | H | H | A-19 | H | H |
| 38607 | 1 | H | H | m-D8 | H | H | H | H | A-20 | H | H |
| 38608 | 1 | H | H | m-D8 | H | H | H | H | A-21 | H | H |
| 38609 | 1 | H | H | m-D8 | H | H | H | H | A-22 | H | H |
| 38610 | 1 | H | H | m-D8 | H | H | H | H | A-23 | H | H |
| 38611 | 1 | H | H | m-D8 | H | H | H | H | A-24 | H | H |
| 38612 | 1 | H | H | m-D8 | H | H | H | H | A-25 | H | H |
| 38613 | 1 | H | H | m-D8 | H | H | H | H | A-26 | H | H |
| 38614 | 1 | H | H | m-D8 | H | H | H | H | A-27 | H | H |
| 38615 | 1 | H | H | m-D8 | H | H | H | H | A-28 | H | H |
| 38616 | 1 | H | H | m-D8 | H | H | H | H | A-29 | H | H |
| 38617 | 1 | H | H | m-D8 | H | H | H | H | A-30 | H | H |
| 38618 | 1 | H | H | m-D8 | H | H | H | H | A-31 | H | H |
| 38619 | 1 | H | H | m-D8 | H | H | H | H | A-32 | H | H |
| 38620 | 1 | H | H | m-D8 | H | H | H | H | A-33 | H | H |
| 38621 | 1 | H | H | m-D8 | H | H | H | H | A-34 | H | H |
| 38622 | 1 | H | H | m-D8 | H | H | H | H | A-35 | H | H |
| 38623 | 1 | H | H | m-D8 | H | H | H | H | A-36 | H | H |
| 38624 | 1 | H | H | m-D8 | H | H | H | H | A-37 | H | H |
| 38625 | 1 | H | H | m-D8 | H | H | H | H | A-38 | H | H |
| 38626 | 1 | H | H | m-D8 | H | H | H | H | A-39 | H | H |
| 38627 | 1 | H | H | m-D8 | H | H | H | H | A-40 | H | H |
| 38628 | 1 | H | H | m-D8 | H | H | H | H | A-41 | H | H |
| 38629 | 1 | H | H | m-D8 | H | H | H | H | A-42 | H | H |
| 38630 | 1 | H | H | m-D8 | H | H | H | H | A-43 | H | H |
| 38631 | 1 | H | H | m-D8 | H | H | H | H | A-44 | H | H |
| 38632 | 1 | H | H | m-D8 | H | H | H | H | A-45 | H | H |
| 38633 | 1 | H | H | m-D8 | H | H | H | H | A-46 | H | H |
| 38634 | 1 | H | H | m-D8 | H | H | H | H | A-47 | H | H |
| 38635 | 1 | H | H | m-D9 | H | H | H | H | A-1 | H | H |
| 38636 | 1 | H | H | m-D9 | H | H | H | H | A-2 | H | H |
| 38637 | 1 | H | H | m-D9 | H | H | H | H | A-3 | H | H |
| 38638 | 1 | H | H | m-D9 | H | H | H | H | A-4 | H | H |
| 38639 | 1 | H | H | m-D9 | H | H | H | H | A-5 | H | H |
| 38640 | 1 | H | H | m-D9 | H | H | H | H | A-6 | H | H |
| 38641 | 1 | H | H | m-D9 | H | H | H | H | A-7 | H | H |
| 38642 | 1 | H | H | m-D9 | H | H | H | H | A-8 | H | H |
| 38643 | 1 | H | H | m-D9 | H | H | H | H | A-9 | H | H |
| 38644 | 1 | H | H | m-D9 | H | H | H | H | A-10 | H | H |
| 38645 | 1 | H | H | m-D9 | H | H | H | H | A-11 | H | H |
| 38646 | 1 | H | H | m-D9 | H | H | H | H | A-12 | H | H |
| 38647 | 1 | H | H | m-D9 | H | H | H | H | A-13 | H | H |
| 38648 | 1 | H | H | m-D9 | H | H | H | H | A-14 | H | H |
| 38649 | 1 | H | H | m-D9 | H | H | H | H | A-15 | H | H |
| 38650 | 1 | H | H | m-D9 | H | H | H | H | A-16 | H | H |
| 38651 | 1 | H | H | m-D9 | H | H | H | H | A-17 | H | H |
| 38652 | 1 | H | H | m-D9 | H | H | H | H | A-18 | H | H |
| 38653 | 1 | H | H | m-D9 | H | H | H | H | A-19 | H | H |
| 38654 | 1 | H | H | m-D9 | H | H | H | H | A-20 | H | H |
| 38655 | 1 | H | H | m-D9 | H | H | H | H | A-21 | H | H |
| 38656 | 1 | H | H | m-D9 | H | H | H | H | A-22 | H | H |
| 38657 | 1 | H | H | m-D9 | H | H | H | H | A-23 | H | H |
| 38658 | 1 | H | H | m-D9 | H | H | H | H | A-24 | H | H |
| 38659 | 1 | H | H | m-D9 | H | H | H | H | A-25 | H | H |
| 38660 | 1 | H | H | m-D9 | H | H | H | H | A-26 | H | H |
| 38661 | 1 | H | H | m-D9 | H | H | H | H | A-27 | H | H |
| 38662 | 1 | H | H | m-D9 | H | H | H | H | A-28 | H | H |
| 38663 | 1 | H | H | m-D9 | H | H | H | H | A-29 | H | H |
| 38664 | 1 | H | H | m-D9 | H | H | H | H | A-30 | H | H |
| 38665 | 1 | H | H | m-D9 | H | H | H | H | A-31 | H | H |
| 38666 | 1 | H | H | m-D9 | H | H | H | H | A-32 | H | H |
| 38667 | 1 | H | H | m-D9 | H | H | H | H | A-33 | H | H |
| 38668 | 1 | H | H | m-D9 | H | H | H | H | A-34 | H | H |
| 38669 | 1 | H | H | m-D9 | H | H | H | H | A-35 | H | H |
| 38670 | 1 | H | H | m-D9 | H | H | H | H | A-36 | H | H |
| 38671 | 1 | H | H | m-D9 | H | H | H | H | A-37 | H | H |
| 38672 | 1 | H | H | m-D9 | H | H | H | H | A-38 | H | H |
| 38673 | 1 | H | H | m-D9 | H | H | H | H | A-39 | H | H |
| 38674 | 1 | H | H | m-D9 | H | H | H | H | A-40 | H | H |
| 38675 | 1 | H | H | m-D9 | H | H | H | H | A-41 | H | H |
| 38676 | 1 | H | H | m-D9 | H | H | H | H | A-42 | H | H |
| 38677 | 1 | H | H | m-D9 | H | H | H | H | A-43 | H | H |
| 38678 | 1 | H | H | m-D9 | H | H | H | H | A-44 | H | H |
| 38679 | 1 | H | H | m-D9 | H | H | H | H | A-45 | H | H |
| 38680 | 1 | H | H | m-D9 | H | H | H | H | A-46 | H | H |
| 38681 | 1 | H | H | m-D9 | H | H | H | H | A-47 | H | H |
| 38682 | 1 | H | H | m-D10 | H | H | H | H | A-1 | H | H |
| 38683 | 1 | H | H | m-D10 | H | H | H | H | A-2 | H | H |
| 38684 | 1 | H | H | m-D10 | H | H | H | H | A-3 | H | H |
| 38685 | 1 | H | H | m-D10 | H | H | H | H | A-4 | H | H |
| 38686 | 1 | H | H | m-D10 | H | H | H | H | A-5 | H | H |
| 38687 | 1 | H | H | m-D10 | H | H | H | H | A-6 | H | H |
| 38688 | 1 | H | H | m-D10 | H | H | H | H | A-7 | H | H |
| 38689 | 1 | H | H | m-D10 | H | H | H | H | A-8 | H | H |
| 38690 | 1 | H | H | m-D10 | H | H | H | H | A-9 | H | H |
| 38691 | 1 | H | H | m-D10 | H | H | H | H | A-10 | H | H |
| 38692 | 1 | H | H | m-D10 | H | H | H | H | A-11 | H | H |
| 38693 | 1 | H | H | m-D10 | H | H | H | H | A-12 | H | H |
| 38694 | 1 | H | H | m-D10 | H | H | H | H | A-13 | H | H |
| 38695 | 1 | H | H | m-D10 | H | H | H | H | A-14 | H | H |
| 38696 | 1 | H | H | m-D10 | H | H | H | H | A-15 | H | H |
| 38697 | 1 | H | H | m-D10 | H | H | H | H | A-16 | H | H |
| 38698 | 1 | H | H | m-D10 | H | H | H | H | A-17 | H | H |
| 38699 | 1 | H | H | m-D10 | H | H | H | H | A-18 | H | H |
| 38700 | 1 | H | H | m-D10 | H | H | H | H | A-19 | H | H |
| 38701 | 1 | H | H | m-D10 | H | H | H | H | A-20 | H | H |
| 38702 | 1 | H | H | m-D10 | H | H | H | H | A-21 | H | H |
| 38703 | 1 | H | H | m-D10 | H | H | H | H | A-22 | H | H |
| 38704 | 1 | H | H | m-D10 | H | H | H | H | A-23 | H | H |
| 38705 | 1 | H | H | m-D10 | H | H | H | H | A-24 | H | H |
| 38706 | 1 | H | H | m-D10 | H | H | H | H | A-25 | H | H |
| 38707 | 1 | H | H | m-D10 | H | H | H | H | A-26 | H | H |
| 38708 | 1 | H | H | m-D10 | H | H | H | H | A-27 | H | H |
| 38709 | 1 | H | H | m-D10 | H | H | H | H | A-28 | H | H |
| 38710 | 1 | H | H | m-D10 | H | H | H | H | A-29 | H | H |
| 38711 | 1 | H | H | m-D10 | H | H | H | H | A-30 | H | H |
| 38712 | 1 | H | H | m-D10 | H | H | H | H | A-31 | H | H |
| 38713 | 1 | H | H | m-D10 | H | H | H | H | A-32 | H | H |
| 38714 | 1 | H | H | m-D10 | H | H | H | H | A-33 | H | H |
| 38715 | 1 | H | H | m-D10 | H | H | H | H | A-34 | H | H |
| 38716 | 1 | H | H | m-D10 | H | H | H | H | A-35 | H | H |
| 38717 | 1 | H | H | m-D10 | H | H | H | H | A-36 | H | H |
| 38718 | 1 | H | H | m-D10 | H | H | H | H | A-37 | H | H |
| 38719 | 1 | H | H | m-D10 | H | H | H | H | A-38 | H | H |
| 38720 | 1 | H | H | m-D10 | H | H | H | H | A-39 | H | H |
| 38721 | 1 | H | H | m-D10 | H | H | H | H | A-40 | H | H |
| 38722 | 1 | H | H | m-D10 | H | H | H | H | A-41 | H | H |
| 38723 | 1 | H | H | m-D10 | H | H | H | H | A-42 | H | H |
| 38724 | 1 | H | H | m-D10 | H | H | H | H | A-43 | H | H |
| 38725 | 1 | H | H | m-D10 | H | H | H | H | A-44 | H | H |
| 38726 | 1 | H | H | m-D10 | H | H | H | H | A-45 | H | H |
| 38727 | 1 | H | H | m-D10 | H | H | H | H | A-46 | H | H |
| 38728 | 1 | H | H | m-D10 | H | H | H | H | A-47 | H | H |
| 38729 | 1 | H | H | m-D11 | H | H | H | H | A-1 | H | H |
| 38730 | 1 | H | H | m-D11 | H | H | H | H | A-2 | H | H |
| 38731 | 1 | H | H | m-D11 | H | H | H | H | A-3 | H | H |
| 38732 | 1 | H | H | m-D11 | H | H | H | H | A-4 | H | H |
| 38733 | 1 | H | H | m-D11 | H | H | H | H | A-5 | H | H |
| 38734 | 1 | H | H | m-D11 | H | H | H | H | A-6 | H | H |
| 38735 | 1 | H | H | m-D11 | H | H | H | H | A-7 | H | H |
| 38736 | 1 | H | H | m-D11 | H | H | H | H | A-8 | H | H |
| 38737 | 1 | H | H | m-D11 | H | H | H | H | A-9 | H | H |
| 38738 | 1 | H | H | m-D11 | H | H | H | H | A-10 | H | H |
| 38739 | 1 | H | H | m-D11 | H | H | H | H | A-11 | H | H |
| 38740 | 1 | H | H | m-D11 | H | H | H | H | A-12 | H | H |
| 38741 | 1 | H | H | m-D11 | H | H | H | H | A-13 | H | H |
| 38742 | 1 | H | H | m-D11 | H | H | H | H | A-14 | H | H |
| 38743 | 1 | H | H | m-D11 | H | H | H | H | A-15 | H | H |
| 38744 | 1 | H | H | m-D11 | H | H | H | H | A-16 | H | H |
| 38745 | 1 | H | H | m-D11 | H | H | H | H | A-17 | H | H |
| 38746 | 1 | H | H | m-D11 | H | H | H | H | A-18 | H | H |
| 38747 | 1 | H | H | m-D11 | H | H | H | H | A-19 | H | H |
| 38748 | 1 | H | H | m-D11 | H | H | H | H | A-20 | H | H |
| 38749 | 1 | H | H | m-D11 | H | H | H | H | A-21 | H | H |
| 38750 | 1 | H | H | m-D11 | H | H | H | H | A-22 | H | H |
| 38751 | 1 | H | H | m-D11 | H | H | H | H | A-23 | H | H |
| 38752 | 1 | H | H | m-D11 | H | H | H | H | A-24 | H | H |
| 38753 | 1 | H | H | m-D11 | H | H | H | H | A-25 | H | H |
| 38754 | 1 | H | H | m-D11 | H | H | H | H | A-26 | H | H |
| 38755 | 1 | H | H | m-D11 | H | H | H | H | A-27 | H | H |
| 38756 | 1 | H | H | m-D11 | H | H | H | H | A-28 | H | H |
| 38757 | 1 | H | H | m-D11 | H | H | H | H | A-29 | H | H |
| 38758 | 1 | H | H | m-D11 | H | H | H | H | A-30 | H | H |
| 38759 | 1 | H | H | m-D11 | H | H | H | H | A-31 | H | H |
| 38760 | 1 | H | H | m-D11 | H | H | H | H | A-32 | H | H |
| 38761 | 1 | H | H | m-D11 | H | H | H | H | A-33 | H | H |
| 38762 | 1 | H | H | m-D11 | H | H | H | H | A-34 | H | H |
| 38763 | 1 | H | H | m-D11 | H | H | H | H | A-35 | H | H |
| 38764 | 1 | H | H | m-D11 | H | H | H | H | A-36 | H | H |
| 38765 | 1 | H | H | m-D11 | H | H | H | H | A-37 | H | H |
| 38766 | 1 | H | H | m-D11 | H | H | H | H | A-38 | H | H |
| 38767 | 1 | H | H | m-D11 | H | H | H | H | A-39 | H | H |
| 38768 | 1 | H | H | m-D11 | H | H | H | H | A-40 | H | H |
| 38769 | 1 | H | H | m-D11 | H | H | H | H | A-41 | H | H |
| 38770 | 1 | H | H | m-D11 | H | H | H | H | A-42 | H | H |
| 38771 | 1 | H | H | m-D11 | H | H | H | H | A-43 | H | H |
| 38772 | 1 | H | H | m-D11 | H | H | H | H | A-44 | H | H |
| 38773 | 1 | H | H | m-D11 | H | H | H | H | A-45 | H | H |
| 38774 | 1 | H | H | m-D11 | H | H | H | H | A-46 | H | H |
| 38775 | 1 | H | H | m-D11 | H | H | H | H | A-47 | H | H |
| 38776 | 1 | H | H | m-D12 | H | H | H | H | A-1 | H | H |
| 38777 | 1 | H | H | m-D12 | H | H | H | H | A-2 | H | H |
| 38778 | 1 | H | H | m-D12 | H | H | H | H | A-3 | H | H |
| 38779 | 1 | H | H | m-D12 | H | H | H | H | A-4 | H | H |
| 38780 | 1 | H | H | m-D12 | H | H | H | H | A-5 | H | H |
| 38781 | 1 | H | H | m-D12 | H | H | H | H | A-6 | H | H |
| 38782 | 1 | H | H | m-D12 | H | H | H | H | A-7 | H | H |
| 38783 | 1 | H | H | m-D12 | H | H | H | H | A-8 | H | H |
| 38784 | 1 | H | H | m-D12 | H | H | H | H | A-9 | H | H |
| 38785 | 1 | H | H | m-D12 | H | H | H | H | A-10 | H | H |
| 38786 | 1 | H | H | m-D12 | H | H | H | H | A-11 | H | H |
| 38787 | 1 | H | H | m-D12 | H | H | H | H | A-12 | H | H |
| 38788 | 1 | H | H | m-D12 | H | H | H | H | A-13 | H | H |
| 38789 | 1 | H | H | m-D12 | H | H | H | H | A-14 | H | H |
| 38790 | 1 | H | H | m-D12 | H | H | H | H | A-15 | H | H |
| 38791 | 1 | H | H | m-D12 | H | H | H | H | A-16 | H | H |
| 38792 | 1 | H | H | m-D12 | H | H | H | H | A-17 | H | H |
| 38793 | 1 | H | H | m-D12 | H | H | H | H | A-18 | H | H |
| 38794 | 1 | H | H | m-D12 | H | H | H | H | A-19 | H | H |
| 38795 | 1 | H | H | m-D12 | H | H | H | H | A-20 | H | H |
| 38796 | 1 | H | H | m-D12 | H | H | H | H | A-21 | H | H |
| 38797 | 1 | H | H | m-D12 | H | H | H | H | A-22 | H | H |
| 38798 | 1 | H | H | m-D12 | H | H | H | H | A-23 | H | H |
| 38799 | 1 | H | H | m-D12 | H | H | H | H | A-24 | H | H |
| 38800 | 1 | H | H | m-D12 | H | H | H | H | A-25 | H | H |
| 38801 | 1 | H | H | m-D12 | H | H | H | H | A-26 | H | H |
| 38802 | 1 | H | H | m-D12 | H | H | H | H | A-27 | H | H |
| 38803 | 1 | H | H | m-D12 | H | H | H | H | A-28 | H | H |
| 38804 | 1 | H | H | m-D12 | H | H | H | H | A-29 | H | H |
| 38805 | 1 | H | H | m-D12 | H | H | H | H | A-30 | H | H |
| 38806 | 1 | H | H | m-D12 | H | H | H | H | A-31 | H | H |
| 38807 | 1 | H | H | m-D12 | H | H | H | H | A-32 | H | H |
| 38808 | 1 | H | H | m-D12 | H | H | H | H | A-33 | H | H |
| 38809 | 1 | H | H | m-D12 | H | H | H | H | A-34 | H | H |
| 38810 | 1 | H | H | m-D12 | H | H | H | H | A-35 | H | H |
| 38811 | 1 | H | H | m-D12 | H | H | H | H | A-36 | H | H |
| 38812 | 1 | H | H | m-D12 | H | H | H | H | A-37 | H | H |
| 38813 | 1 | H | H | m-D12 | H | H | H | H | A-38 | H | H |
| 38814 | 1 | H | H | m-D12 | H | H | H | H | A-39 | H | H |
| 38815 | 1 | H | H | m-D12 | H | H | H | H | A-40 | H | H |
| 38816 | 1 | H | H | m-D12 | H | H | H | H | A-41 | H | H |
| 38817 | 1 | H | H | m-D12 | H | H | H | H | A-42 | H | H |
| 38818 | 1 | H | H | m-D12 | H | H | H | H | A-43 | H | H |
| 38819 | 1 | H | H | m-D12 | H | H | H | H | A-44 | H | H |
| 38820 | 1 | H | H | m-D12 | H | H | H | H | A-45 | H | H |
| 38821 | 1 | H | H | m-D12 | H | H | H | H | A-46 | H | H |
| 38822 | 1 | H | H | m-D12 | H | H | H | H | A-47 | H | H |
| 38823 | 1 | H | H | m-D13 | H | H | H | H | A-1 | H | H |
| 38824 | 1 | H | H | m-D13 | H | H | H | H | A-2 | H | H |
| 38825 | 1 | H | H | m-D13 | H | H | H | H | A-3 | H | H |
| 38826 | 1 | H | H | m-D13 | H | H | H | H | A-4 | H | H |
| 38827 | 1 | H | H | m-D13 | H | H | H | H | A-5 | H | H |
| 38828 | 1 | H | H | m-D13 | H | H | H | H | A-6 | H | H |
| 38829 | 1 | H | H | m-D13 | H | H | H | H | A-7 | H | H |
| 38830 | 1 | H | H | m-D13 | H | H | H | H | A-8 | H | H |
| 38831 | 1 | H | H | m-D13 | H | H | H | H | A-9 | H | H |
| 38832 | 1 | H | H | m-D13 | H | H | H | H | A-10 | H | H |
| 38833 | 1 | H | H | m-D13 | H | H | H | H | A-11 | H | H |
| 38834 | 1 | H | H | m-D13 | H | H | H | H | A-12 | H | H |
| 38835 | 1 | H | H | m-D13 | H | H | H | H | A-13 | H | H |
| 38836 | 1 | H | H | m-D13 | H | H | H | H | A-14 | H | H |
| 38837 | 1 | H | H | m-D13 | H | H | H | H | A-15 | H | H |
| 38838 | 1 | H | H | m-D13 | H | H | H | H | A-16 | H | H |
| 38839 | 1 | H | H | m-D13 | H | H | H | H | A-17 | H | H |
| 38840 | 1 | H | H | m-D13 | H | H | H | H | A-18 | H | H |
| 38841 | 1 | H | H | m-D13 | H | H | H | H | A-19 | H | H |
| 38842 | 1 | H | H | m-D13 | H | H | H | H | A-20 | H | H |
| 38843 | 1 | H | H | m-D13 | H | H | H | H | A-21 | H | H |
| 38844 | 1 | H | H | m-D13 | H | H | H | H | A-22 | H | H |
| 38845 | 1 | H | H | m-D13 | H | H | H | H | A-23 | H | H |
| 38846 | 1 | H | H | m-D13 | H | H | H | H | A-24 | H | H |
| 38847 | 1 | H | H | m-D13 | H | H | H | H | A-25 | H | H |
| 38848 | 1 | H | H | m-D13 | H | H | H | H | A-26 | H | H |
| 38849 | 1 | H | H | m-D13 | H | H | H | H | A-27 | H | H |
| 38850 | 1 | H | H | m-D13 | H | H | H | H | A-28 | H | H |
| 38851 | 1 | H | H | m-D13 | H | H | H | H | A-29 | H | H |
| 38852 | 1 | H | H | m-D13 | H | H | H | H | A-30 | H | H |
| 38853 | 1 | H | H | m-D13 | H | H | H | H | A-31 | H | H |
| 38854 | 1 | H | H | m-D13 | H | H | H | H | A-32 | H | H |
| 38855 | 1 | H | H | m-D13 | H | H | H | H | A-33 | H | H |
| 38856 | 1 | H | H | m-D13 | H | H | H | H | A-34 | H | H |
| 38857 | 1 | H | H | m-D13 | H | H | H | H | A-35 | H | H |
| 38858 | 1 | H | H | m-D13 | H | H | H | H | A-36 | H | H |
| 38859 | 1 | H | H | m-D13 | H | H | H | H | A-37 | H | H |
| 38860 | 1 | H | H | m-D13 | H | H | H | H | A-38 | H | H |
| 38861 | 1 | H | H | m-D13 | H | H | H | H | A-39 | H | H |
| 38862 | 1 | H | H | m-D13 | H | H | H | H | A-40 | H | H |
| 38863 | 1 | H | H | m-D13 | H | H | H | H | A-41 | H | H |
| 38864 | 1 | H | H | m-D13 | H | H | H | H | A-42 | H | H |
| 38865 | 1 | H | H | m-D13 | H | H | H | H | A-43 | H | H |
| 38866 | 1 | H | H | m-D13 | H | H | H | H | A-44 | H | H |
| 38867 | 1 | H | H | m-D13 | H | H | H | H | A-45 | H | H |
| 38868 | 1 | H | H | m-D13 | H | H | H | H | A-46 | H | H |
| 38869 | 1 | H | H | m-D13 | H | H | H | H | A-47 | H | H |
| 38870 | 1 | H | H | m-D14 | H | H | H | H | A-1 | H | H |
| 38871 | 1 | H | H | m-D14 | H | H | H | H | A-2 | H | H |
| 38872 | 1 | H | H | m-D14 | H | H | H | H | A-3 | H | H |
| 38873 | 1 | H | H | m-D14 | H | H | H | H | A-4 | H | H |
| 38874 | 1 | H | H | m-D14 | H | H | H | H | A-5 | H | H |
| 38875 | 1 | H | H | m-D14 | H | H | H | H | A-6 | H | H |
| 38876 | 1 | H | H | m-D14 | H | H | H | H | A-7 | H | H |
| 38877 | 1 | H | H | m-D14 | H | H | H | H | A-8 | H | H |
| 38878 | 1 | H | H | m-D14 | H | H | H | H | A-9 | H | H |
| 38879 | 1 | H | H | m-D14 | H | H | H | H | A-10 | H | H |
| 38880 | 1 | H | H | m-D14 | H | H | H | H | A-11 | H | H |
| 38881 | 1 | H | H | m-D14 | H | H | H | H | A-12 | H | H |
| 38882 | 1 | H | H | m-D14 | H | H | H | H | A-13 | H | H |
| 38883 | 1 | H | H | m-D14 | H | H | H | H | A-14 | H | H |
| 38884 | 1 | H | H | m-D14 | H | H | H | H | A-15 | H | H |
| 38885 | 1 | H | H | m-D14 | H | H | H | H | A-16 | H | H |
| 38886 | 1 | H | H | m-D14 | H | H | H | H | A-17 | H | H |
| 38887 | 1 | H | H | m-D14 | H | H | H | H | A-18 | H | H |
| 38888 | 1 | H | H | m-D14 | H | H | H | H | A-19 | H | H |
| 38889 | 1 | H | H | m-D14 | H | H | H | H | A-20 | H | H |
| 38890 | 1 | H | H | m-D14 | H | H | H | H | A-21 | H | H |
| 38891 | 1 | H | H | m-D14 | H | H | H | H | A-22 | H | H |
| 38892 | 1 | H | H | m-D14 | H | H | H | H | A-23 | H | H |
| 38893 | 1 | H | H | m-D14 | H | H | H | H | A-24 | H | H |
| 38894 | 1 | H | H | m-D14 | H | H | H | H | A-25 | H | H |
| 38895 | 1 | H | H | m-D14 | H | H | H | H | A-26 | H | H |
| 38896 | 1 | H | H | m-D14 | H | H | H | H | A-27 | H | H |
| 38897 | 1 | H | H | m-D14 | H | H | H | H | A-28 | H | H |
| 38898 | 1 | H | H | m-D14 | H | H | H | H | A-29 | H | H |
| 38899 | 1 | H | H | m-D14 | H | H | H | H | A-30 | H | H |
| 38900 | 1 | H | H | m-D14 | H | H | H | H | A-31 | H | H |
| 38901 | 1 | H | H | m-D14 | H | H | H | H | A-32 | H | H |
| 38902 | 1 | H | H | m-D14 | H | H | H | H | A-33 | H | H |
| 38903 | 1 | H | H | m-D14 | H | H | H | H | A-34 | H | H |
| 38904 | 1 | H | H | m-D14 | H | H | H | H | A-35 | H | H |
| 38905 | 1 | H | H | m-D14 | H | H | H | H | A-36 | H | H |
| 38906 | 1 | H | H | m-D14 | H | H | H | H | A-37 | H | H |
| 38907 | 1 | H | H | m-D14 | H | H | H | H | A-38 | H | H |
| 38908 | 1 | H | H | m-D14 | H | H | H | H | A-39 | H | H |
| 38909 | 1 | H | H | m-D14 | H | H | H | H | A-40 | H | H |
| 38910 | 1 | H | H | m-D14 | H | H | H | H | A-41 | H | H |
| 38911 | 1 | H | H | m-D14 | H | H | H | H | A-42 | H | H |
| 38912 | 1 | H | H | m-D14 | H | H | H | H | A-43 | H | H |
| 38913 | 1 | H | H | m-D14 | H | H | H | H | A-44 | H | H |
| 38914 | 1 | H | H | m-D14 | H | H | H | H | A-45 | H | H |
| 38915 | 1 | H | H | m-D14 | H | H | H | H | A-46 | H | H |
| 38916 | 1 | H | H | m-D14 | H | H | H | H | A-47 | H | H |
| 38917 | 1 | H | H | m-D1 | H | H | H | A-1 | H | H | H |
| 38918 | 1 | H | H | m-D1 | H | H | H | A-2 | H | H | H |
| 38919 | 1 | H | H | m-D1 | H | H | H | A-3 | H | H | H |
| 38920 | 1 | H | H | m-D1 | H | H | H | A-4 | H | H | H |
| 38921 | 1 | H | H | m-D1 | H | H | H | A-5 | H | H | H |
| 38922 | 1 | H | H | m-D1 | H | H | H | A-6 | H | H | H |
| 38923 | 1 | H | H | m-D1 | H | H | H | A-7 | H | H | H |
| 38924 | 1 | H | H | m-D1 | H | H | H | A-8 | H | H | H |
| 38925 | 1 | H | H | m-D1 | H | H | H | A-9 | H | H | H |
| 38926 | 1 | H | H | m-D1 | H | H | H | A-10 | H | H | H |
| 38927 | 1 | H | H | m-D1 | H | H | H | A-11 | H | H | H |
| 38928 | 1 | H | H | m-D1 | H | H | H | A-12 | H | H | H |
| 38929 | 1 | H | H | m-D1 | H | H | H | A-13 | H | H | H |
| 38930 | 1 | H | H | m-D1 | H | H | H | A-14 | H | H | H |
| 38931 | 1 | H | H | m-D1 | H | H | H | A-15 | H | H | H |
| 38932 | 1 | H | H | m-D1 | H | H | H | A-16 | H | H | H |
| 38933 | 1 | H | H | m-D1 | H | H | H | A-17 | H | H | H |
| 38934 | 1 | H | H | m-D1 | H | H | H | A-18 | H | H | H |
| 38935 | 1 | H | H | m-D1 | H | H | H | A-19 | H | H | H |
| 38936 | 1 | H | H | m-D1 | H | H | H | A-20 | H | H | H |
| 38937 | 1 | H | H | m-D1 | H | H | H | A-21 | H | H | H |
| 38938 | 1 | H | H | m-D1 | H | H | H | A-22 | H | H | H |
| 38939 | 1 | H | H | m-D1 | H | H | H | A-23 | H | H | H |
| 38940 | 1 | H | H | m-D1 | H | H | H | A-24 | H | H | H |
| 38941 | 1 | H | H | m-D1 | H | H | H | A-25 | H | H | H |
| 38942 | 1 | H | H | m-D1 | H | H | H | A-26 | H | H | H |
| 38943 | 1 | H | H | m-D1 | H | H | H | A-27 | H | H | H |
| 38944 | 1 | H | H | m-D1 | H | H | H | A-28 | H | H | H |
| 38945 | 1 | H | H | m-D1 | H | H | H | A-29 | H | H | H |
| 38946 | 1 | H | H | m-D1 | H | H | H | A-30 | H | H | H |
| 38947 | 1 | H | H | m-D1 | H | H | H | A-31 | H | H | H |
| 38948 | 1 | H | H | m-D1 | H | H | H | A-32 | H | H | H |
| 38949 | 1 | H | H | m-D1 | H | H | H | A-33 | H | H | H |
| 38950 | 1 | H | H | m-D1 | H | H | H | A-34 | H | H | H |
| 38951 | 1 | H | H | m-D1 | H | H | H | A-35 | H | H | H |
| 38952 | 1 | H | H | m-D1 | H | H | H | A-36 | H | H | H |
| 38953 | 1 | H | H | m-D1 | H | H | H | A-37 | H | H | H |
| 38954 | 1 | H | H | m-D1 | H | H | H | A-38 | H | H | H |
| 38955 | 1 | H | H | m-D1 | H | H | H | A-39 | H | H | H |
| 38956 | 1 | H | H | m-D1 | H | H | H | A-40 | H | H | H |
| 38957 | 1 | H | H | m-D1 | H | H | H | A-41 | H | H | H |
| 38958 | 1 | H | H | m-D1 | H | H | H | A-42 | H | H | H |
| 38959 | 1 | H | H | m-D1 | H | H | H | A-43 | H | H | H |
| 38960 | 1 | H | H | m-D1 | H | H | H | A-44 | H | H | H |
| 38961 | 1 | H | H | m-D1 | H | H | H | A-45 | H | H | H |
| 38962 | 1 | H | H | m-D1 | H | H | H | A-46 | H | H | H |
| 38963 | 1 | H | H | m-D1 | H | H | H | A-47 | H | H | H |
| 38964 | 1 | H | H | m-D2 | H | H | H | A-1 | H | H | H |
| 38965 | 1 | H | H | m-D2 | H | H | H | A-2 | H | H | H |
| 38966 | 1 | H | H | m-D2 | H | H | H | A-3 | H | H | H |
| 38967 | 1 | H | H | m-D2 | H | H | H | A-4 | H | H | H |
| 38968 | 1 | H | H | m-D2 | H | H | H | A-5 | H | H | H |
| 38969 | 1 | H | H | m-D2 | H | H | H | A-6 | H | H | H |
| 38970 | 1 | H | H | m-D2 | H | H | H | A-7 | H | H | H |
| 38971 | 1 | H | H | m-D2 | H | H | H | A-8 | H | H | H |
| 38972 | 1 | H | H | m-D2 | H | H | H | A-9 | H | H | H |
| 38973 | 1 | H | H | m-D2 | H | H | H | A-10 | H | H | H |
| 38974 | 1 | H | H | m-D2 | H | H | H | A-11 | H | H | H |
| 38975 | 1 | H | H | m-D2 | H | H | H | A-12 | H | H | H |
| 38976 | 1 | H | H | m-D2 | H | H | H | A-13 | H | H | H |
| 38977 | 1 | H | H | m-D2 | H | H | H | A-14 | H | H | H |
| 38978 | 1 | H | H | m-D2 | H | H | H | A-15 | H | H | H |
| 38979 | 1 | H | H | m-D2 | H | H | H | A-16 | H | H | H |
| 38980 | 1 | H | H | m-D2 | H | H | H | A-17 | H | H | H |
| 38981 | 1 | H | H | m-D2 | H | H | H | A-18 | H | H | H |
| 38982 | 1 | H | H | m-D2 | H | H | H | A-19 | H | H | H |
| 38983 | 1 | H | H | m-D2 | H | H | H | A-20 | H | H | H |
| 38984 | 1 | H | H | m-D2 | H | H | H | A-21 | H | H | H |
| 38985 | 1 | H | H | m-D2 | H | H | H | A-22 | H | H | H |
| 38986 | 1 | H | H | m-D2 | H | H | H | A-23 | H | H | H |
| 38987 | 1 | H | H | m-D2 | H | H | H | A-24 | H | H | H |
| 38988 | 1 | H | H | m-D2 | H | H | H | A-25 | H | H | H |
| 38989 | 1 | H | H | m-D2 | H | H | H | A-26 | H | H | H |
| 38990 | 1 | H | H | m-D2 | H | H | H | A-27 | H | H | H |
| 38991 | 1 | H | H | m-D2 | H | H | H | A-28 | H | H | H |
| 38992 | 1 | H | H | m-D2 | H | H | H | A-29 | H | H | H |
| 38993 | 1 | H | H | m-D2 | H | H | H | A-30 | H | H | H |
| 38994 | 1 | H | H | m-D2 | H | H | H | A-31 | H | H | H |
| 38995 | 1 | H | H | m-D2 | H | H | H | A-32 | H | H | H |
| 38996 | 1 | H | H | m-D2 | H | H | H | A-33 | H | H | H |
| 38997 | 1 | H | H | m-D2 | H | H | H | A-34 | H | H | H |
| 38998 | 1 | H | H | m-D2 | H | H | H | A-35 | H | H | H |
| 38999 | 1 | H | H | m-D2 | H | H | H | A-36 | H | H | H |
| 39000 | 1 | H | H | m-D2 | H | H | H | A-37 | H | H | H |
| 39001 | 1 | H | H | m-D2 | H | H | H | A-38 | H | H | H |
| 39002 | 1 | H | H | m-D2 | H | H | H | A-39 | H | H | H |
| 39003 | 1 | H | H | m-D2 | H | H | H | A-40 | H | H | H |
| 39004 | 1 | H | H | m-D2 | H | H | H | A-41 | H | H | H |
| 39005 | 1 | H | H | m-D2 | H | H | H | A-42 | H | H | H |
| 39006 | 1 | H | H | m-D2 | H | H | H | A-43 | H | H | H |
| 39007 | 1 | H | H | m-D2 | H | H | H | A-44 | H | H | H |
| 39008 | 1 | H | H | m-D2 | H | H | H | A-45 | H | H | H |
| 39009 | 1 | H | H | m-D2 | H | H | H | A-46 | H | H | H |
| 39010 | 1 | H | H | m-D2 | H | H | H | A-47 | H | H | H |
| 39011 | 1 | H | H | m-D3 | H | H | H | A-1 | H | H | H |
| 39012 | 1 | H | H | m-D3 | H | H | H | A-2 | H | H | H |
| 39013 | 1 | H | H | m-D3 | H | H | H | A-3 | H | H | H |
| 39014 | 1 | H | H | m-D3 | H | H | H | A-4 | H | H | H |
| 39015 | 1 | H | H | m-D3 | H | H | H | A-5 | H | H | H |
| 39016 | 1 | H | H | m-D3 | H | H | H | A-6 | H | H | H |
| 39017 | 1 | H | H | m-D3 | H | H | H | A-7 | H | H | H |
| 39018 | 1 | H | H | m-D3 | H | H | H | A-8 | H | H | H |
| 39019 | 1 | H | H | m-D3 | H | H | H | A-9 | H | H | H |
| 39020 | 1 | H | H | m-D3 | H | H | H | A-10 | H | H | H |
| 39021 | 1 | H | H | m-D3 | H | H | H | A-11 | H | H | H |
| 39022 | 1 | H | H | m-D3 | H | H | H | A-12 | H | H | H |
| 39023 | 1 | H | H | m-D3 | H | H | H | A-13 | H | H | H |
| 39024 | 1 | H | H | m-D3 | H | H | H | A-14 | H | H | H |
| 39025 | 1 | H | H | m-D3 | H | H | H | A-15 | H | H | H |
| 39026 | 1 | H | H | m-D3 | H | H | H | A-16 | H | H | H |
| 39027 | 1 | H | H | m-D3 | H | H | H | A-17 | H | H | H |
| 39028 | 1 | H | H | m-D3 | H | H | H | A-18 | H | H | H |
| 39029 | 1 | H | H | m-D3 | H | H | H | A-19 | H | H | H |
| 39030 | 1 | H | H | m-D3 | H | H | H | A-20 | H | H | H |
| 39031 | 1 | H | H | m-D3 | H | H | H | A-21 | H | H | H |
| 39032 | 1 | H | H | m-D3 | H | H | H | A-22 | H | H | H |
| 39033 | 1 | H | H | m-D3 | H | H | H | A-23 | H | H | H |
| 39034 | 1 | H | H | m-D3 | H | H | H | A-24 | H | H | H |
| 39035 | 1 | H | H | m-D3 | H | H | H | A-25 | H | H | H |
| 39036 | 1 | H | H | m-D3 | H | H | H | A-26 | H | H | H |
| 39037 | 1 | H | H | m-D3 | H | H | H | A-27 | H | H | H |
| 39038 | 1 | H | H | m-D3 | H | H | H | A-28 | H | H | H |
| 39039 | 1 | H | H | m-D3 | H | H | H | A-29 | H | H | H |
| 39040 | 1 | H | H | m-D3 | H | H | H | A-30 | H | H | H |
| 39041 | 1 | H | H | m-D3 | H | H | H | A-31 | H | H | H |
| 39042 | 1 | H | H | m-D3 | H | H | H | A-32 | H | H | H |
| 39043 | 1 | H | H | m-D3 | H | H | H | A-33 | H | H | H |
| 39044 | 1 | H | H | m-D3 | H | H | H | A-34 | H | H | H |
| 39045 | 1 | H | H | m-D3 | H | H | H | A-35 | H | H | H |
| 39046 | 1 | H | H | m-D3 | H | H | H | A-36 | H | H | H |
| 39047 | 1 | H | H | m-D3 | H | H | H | A-37 | H | H | H |
| 39048 | 1 | H | H | m-D3 | H | H | H | A-38 | H | H | H |
| 39049 | 1 | H | H | m-D3 | H | H | H | A-39 | H | H | H |
| 39050 | 1 | H | H | m-D3 | H | H | H | A-40 | H | H | H |
| 39051 | 1 | H | H | m-D3 | H | H | H | A-41 | H | H | H |
| 39052 | 1 | H | H | m-D3 | H | H | H | A-42 | H | H | H |
| 39053 | 1 | H | H | m-D3 | H | H | H | A-43 | H | H | H |
| 39054 | 1 | H | H | m-D3 | H | H | H | A-44 | H | H | H |
| 39055 | 1 | H | H | m-D3 | H | H | H | A-45 | H | H | H |
| 39056 | 1 | H | H | m-D3 | H | H | H | A-46 | H | H | H |
| 39057 | 1 | H | H | m-D3 | H | H | H | A-47 | H | H | H |
| 39058 | 1 | H | H | m-D4 | H | H | H | A-1 | H | H | H |
| 39059 | 1 | H | H | m-D4 | H | H | H | A-2 | H | H | H |
| 39060 | 1 | H | H | m-D4 | H | H | H | A-3 | H | H | H |
| 39061 | 1 | H | H | m-D4 | H | H | H | A-4 | H | H | H |
| 39062 | 1 | H | H | m-D4 | H | H | H | A-5 | H | H | H |
| 39063 | 1 | H | H | m-D4 | H | H | H | A-6 | H | H | H |
| 39064 | 1 | H | H | m-D4 | H | H | H | A-7 | H | H | H |
| 39065 | 1 | H | H | m-D4 | H | H | H | A-8 | H | H | H |
| 39066 | 1 | H | H | m-D4 | H | H | H | A-9 | H | H | H |
| 39067 | 1 | H | H | m-D4 | H | H | H | A-10 | H | H | H |
| 39068 | 1 | H | H | m-D4 | H | H | H | A-11 | H | H | H |
| 39069 | 1 | H | H | m-D4 | H | H | H | A-12 | H | H | H |
| 39070 | 1 | H | H | m-D4 | H | H | H | A-13 | H | H | H |
| 39071 | 1 | H | H | m-D4 | H | H | H | A-14 | H | H | H |
| 39072 | 1 | H | H | m-D4 | H | H | H | A-15 | H | H | H |
| 39073 | 1 | H | H | m-D4 | H | H | H | A-16 | H | H | H |
| 39074 | 1 | H | H | m-D4 | H | H | H | A-17 | H | H | H |
| 39075 | 1 | H | H | m-D4 | H | H | H | A-18 | H | H | H |
| 39076 | 1 | H | H | m-D4 | H | H | H | A-19 | H | H | H |
| 39077 | 1 | H | H | m-D4 | H | H | H | A-20 | H | H | H |
| 39078 | 1 | H | H | m-D4 | H | H | H | A-21 | H | H | H |
| 39079 | 1 | H | H | m-D4 | H | H | H | A-22 | H | H | H |
| 39080 | 1 | H | H | m-D4 | H | H | H | A-23 | H | H | H |
| 39081 | 1 | H | H | m-D4 | H | H | H | A-24 | H | H | H |
| 39082 | 1 | H | H | m-D4 | H | H | H | A-25 | H | H | H |
| 39083 | 1 | H | H | m-D4 | H | H | H | A-26 | H | H | H |
| 39084 | 1 | H | H | m-D4 | H | H | H | A-27 | H | H | H |
| 39085 | 1 | H | H | m-D4 | H | H | H | A-28 | H | H | H |
| 39086 | 1 | H | H | m-D4 | H | H | H | A-29 | H | H | H |
| 39087 | 1 | H | H | m-D4 | H | H | H | A-30 | H | H | H |
| 39088 | 1 | H | H | m-D4 | H | H | H | A-31 | H | H | H |
| 39089 | 1 | H | H | m-D4 | H | H | H | A-32 | H | H | H |
| 39090 | 1 | H | H | m-D4 | H | H | H | A-33 | H | H | H |
| 39091 | 1 | H | H | m-D4 | H | H | H | A-34 | H | H | H |
| 39092 | 1 | H | H | m-D4 | H | H | H | A-35 | H | H | H |
| 39093 | 1 | H | H | m-D4 | H | H | H | A-36 | H | H | H |
| 39094 | 1 | H | H | m-D4 | H | H | H | A-37 | H | H | H |
| 39095 | 1 | H | H | m-D4 | H | H | H | A-38 | H | H | H |
| 39096 | 1 | H | H | m-D4 | H | H | H | A-39 | H | H | H |
| 39097 | 1 | H | H | m-D4 | H | H | H | A-40 | H | H | H |
| 39098 | 1 | H | H | m-D4 | H | H | H | A-41 | H | H | H |
| 39099 | 1 | H | H | m-D4 | H | H | H | A-42 | H | H | H |
| 39100 | 1 | H | H | m-D4 | H | H | H | A-43 | H | H | H |
| 39101 | 1 | H | H | m-D4 | H | H | H | A-44 | H | H | H |
| 39102 | 1 | H | H | m-D4 | H | H | H | A-45 | H | H | H |
| 39103 | 1 | H | H | m-D4 | H | H | H | A-46 | H | H | H |
| 39104 | 1 | H | H | m-D4 | H | H | H | A-47 | H | H | H |
| 39105 | 1 | H | H | m-D5 | H | H | H | A-1 | H | H | H |
| 39106 | 1 | H | H | m-D5 | H | H | H | A-2 | H | H | H |
| 39107 | 1 | H | H | m-D5 | H | H | H | A-3 | H | H | H |
| 39108 | 1 | H | H | m-D5 | H | H | H | A-4 | H | H | H |
| 39109 | 1 | H | H | m-D5 | H | H | H | A-5 | H | H | H |
| 39110 | 1 | H | H | m-D5 | H | H | H | A-6 | H | H | H |
| 39111 | 1 | H | H | m-D5 | H | H | H | A-7 | H | H | H |
| 39112 | 1 | H | H | m-D5 | H | H | H | A-8 | H | H | H |
| 39113 | 1 | H | H | m-D5 | H | H | H | A-9 | H | H | H |
| 39114 | 1 | H | H | m-D5 | H | H | H | A-10 | H | H | H |
| 39115 | 1 | H | H | m-D5 | H | H | H | A-11 | H | H | H |
| 39116 | 1 | H | H | m-D5 | H | H | H | A-12 | H | H | H |
| 39117 | 1 | H | H | m-D5 | H | H | H | A-13 | H | H | H |
| 39118 | 1 | H | H | m-D5 | H | H | H | A-14 | H | H | H |
| 39119 | 1 | H | H | m-D5 | H | H | H | A-15 | H | H | H |
| 39120 | 1 | H | H | m-D5 | H | H | H | A-16 | H | H | H |
| 39121 | 1 | H | H | m-D5 | H | H | H | A-17 | H | H | H |
| 39122 | 1 | H | H | m-D5 | H | H | H | A-18 | H | H | H |
| 39123 | 1 | H | H | m-D5 | H | H | H | A-19 | H | H | H |
| 39124 | 1 | H | H | m-D5 | H | H | H | A-20 | H | H | H |
| 39125 | 1 | H | H | m-D5 | H | H | H | A-21 | H | H | H |
| 39126 | 1 | H | H | m-D5 | H | H | H | A-22 | H | H | H |
| 39127 | 1 | H | H | m-D5 | H | H | H | A-23 | H | H | H |
| 39128 | 1 | H | H | m-D5 | H | H | H | A-24 | H | H | H |
| 39129 | 1 | H | H | m-D5 | H | H | H | A-25 | H | H | H |
| 39130 | 1 | H | H | m-D5 | H | H | H | A-26 | H | H | H |
| 39131 | 1 | H | H | m-D5 | H | H | H | A-27 | H | H | H |
| 39132 | 1 | H | H | m-D5 | H | H | H | A-28 | H | H | H |
| 39133 | 1 | H | H | m-D5 | H | H | H | A-29 | H | H | H |
| 39134 | 1 | H | H | m-D5 | H | H | H | A-30 | H | H | H |
| 39135 | 1 | H | H | m-D5 | H | H | H | A-31 | H | H | H |
| 39136 | 1 | H | H | m-D5 | H | H | H | A-32 | H | H | H |
| 39137 | 1 | H | H | m-D5 | H | H | H | A-33 | H | H | H |
| 39138 | 1 | H | H | m-D5 | H | H | H | A-34 | H | H | H |
| 39139 | 1 | H | H | m-D5 | H | H | H | A-35 | H | H | H |
| 39140 | 1 | H | H | m-D5 | H | H | H | A-36 | H | H | H |
| 39141 | 1 | H | H | m-D5 | H | H | H | A-37 | H | H | H |
| 39142 | 1 | H | H | m-D5 | H | H | H | A-38 | H | H | H |
| 39143 | 1 | H | H | m-D5 | H | H | H | A-39 | H | H | H |
| 39144 | 1 | H | H | m-D5 | H | H | H | A-40 | H | H | H |
| 39145 | 1 | H | H | m-D5 | H | H | H | A-41 | H | H | H |
| 39146 | 1 | H | H | m-D5 | H | H | H | A-42 | H | H | H |
| 39147 | 1 | H | H | m-D5 | H | H | H | A-43 | H | H | H |
| 39148 | 1 | H | H | m-D5 | H | H | H | A-44 | H | H | H |
| 39149 | 1 | H | H | m-D5 | H | H | H | A-45 | H | H | H |
| 39150 | 1 | H | H | m-D5 | H | H | H | A-46 | H | H | H |
| 39151 | 1 | H | H | m-D5 | H | H | H | A-47 | H | H | H |
| 39152 | 1 | H | H | m-D6 | H | H | H | A-1 | H | H | H |
| 39153 | 1 | H | H | m-D6 | H | H | H | A-2 | H | H | H |
| 39154 | 1 | H | H | m-D6 | H | H | H | A-3 | H | H | H |
| 39155 | 1 | H | H | m-D6 | H | H | H | A-4 | H | H | H |
| 39156 | 1 | H | H | m-D6 | H | H | H | A-5 | H | H | H |
| 39157 | 1 | H | H | m-D6 | H | H | H | A-6 | H | H | H |
| 39158 | 1 | H | H | m-D6 | H | H | H | A-7 | H | H | H |
| 39159 | 1 | H | H | m-D6 | H | H | H | A-8 | H | H | H |
| 39160 | 1 | H | H | m-D6 | H | H | H | A-9 | H | H | H |
| 39161 | 1 | H | H | m-D6 | H | H | H | A-10 | H | H | H |
| 39162 | 1 | H | H | m-D6 | H | H | H | A-11 | H | H | H |
| 39163 | 1 | H | H | m-D6 | H | H | H | A-12 | H | H | H |
| 39164 | 1 | H | H | m-D6 | H | H | H | A-13 | H | H | H |
| 39165 | 1 | H | H | m-D6 | H | H | H | A-14 | H | H | H |
| 39166 | 1 | H | H | m-D6 | H | H | H | A-15 | H | H | H |
| 39167 | 1 | H | H | m-D6 | H | H | H | A-16 | H | H | H |
| 39168 | 1 | H | H | m-D6 | H | H | H | A-17 | H | H | H |
| 39169 | 1 | H | H | m-D6 | H | H | H | A-18 | H | H | H |
| 39170 | 1 | H | H | m-D6 | H | H | H | A-19 | H | H | H |
| 39171 | 1 | H | H | m-D6 | H | H | H | A-20 | H | H | H |
| 39172 | 1 | H | H | m-D6 | H | H | H | A-21 | H | H | H |
| 39173 | 1 | H | H | m-D6 | H | H | H | A-22 | H | H | H |
| 39174 | 1 | H | H | m-D6 | H | H | H | A-23 | H | H | H |
| 39175 | 1 | H | H | m-D6 | H | H | H | A-24 | H | H | H |
| 39176 | 1 | H | H | m-D6 | H | H | H | A-25 | H | H | H |
| 39177 | 1 | H | H | m-D6 | H | H | H | A-26 | H | H | H |
| 39178 | 1 | H | H | m-D6 | H | H | H | A-27 | H | H | H |
| 39179 | 1 | H | H | m-D6 | H | H | H | A-28 | H | H | H |
| 39180 | 1 | H | H | m-D6 | H | H | H | A-29 | H | H | H |
| 39181 | 1 | H | H | m-D6 | H | H | H | A-30 | H | H | H |
| 39182 | 1 | H | H | m-D6 | H | H | H | A-31 | H | H | H |
| 39183 | 1 | H | H | m-D6 | H | H | H | A-32 | H | H | H |
| 39184 | 1 | H | H | m-D6 | H | H | H | A-33 | H | H | H |
| 39185 | 1 | H | H | m-D6 | H | H | H | A-34 | H | H | H |
| 39186 | 1 | H | H | m-D6 | H | H | H | A-35 | H | H | H |
| 39187 | 1 | H | H | m-D6 | H | H | H | A-36 | H | H | H |
| 39188 | 1 | H | H | m-D6 | H | H | H | A-37 | H | H | H |
| 39189 | 1 | H | H | m-D6 | H | H | H | A-38 | H | H | H |
| 39190 | 1 | H | H | m-D6 | H | H | H | A-39 | H | H | H |
| 39191 | 1 | H | H | m-D6 | H | H | H | A-40 | H | H | H |
| 39192 | 1 | H | H | m-D6 | H | H | H | A-41 | H | H | H |
| 39193 | 1 | H | H | m-D6 | H | H | H | A-42 | H | H | H |
| 39194 | 1 | H | H | m-D6 | H | H | H | A-43 | H | H | H |
| 39195 | 1 | H | H | m-D6 | H | H | H | A-44 | H | H | H |
| 39196 | 1 | H | H | m-D6 | H | H | H | A-45 | H | H | H |
| 39197 | 1 | H | H | m-D6 | H | H | H | A-46 | H | H | H |
| 39198 | 1 | H | H | m-D6 | H | H | H | A-47 | H | H | H |
| 39199 | 1 | H | H | m-D7 | H | H | H | A-1 | H | H | H |
| 39200 | 1 | H | H | m-D7 | H | H | H | A-2 | H | H | H |
| 39201 | 1 | H | H | m-D7 | H | H | H | A-3 | H | H | H |
| 39202 | 1 | H | H | m-D7 | H | H | H | A-4 | H | H | H |
| 39203 | 1 | H | H | m-D7 | H | H | H | A-5 | H | H | H |
| 39204 | 1 | H | H | m-D7 | H | H | H | A-6 | H | H | H |
| 39205 | 1 | H | H | m-D7 | H | H | H | A-7 | H | H | H |
| 39206 | 1 | H | H | m-D7 | H | H | H | A-8 | H | H | H |
| 39207 | 1 | H | H | m-D7 | H | H | H | A-9 | H | H | H |
| 39208 | 1 | H | H | m-D7 | H | H | H | A-10 | H | H | H |
| 39209 | 1 | H | H | m-D7 | H | H | H | A-11 | H | H | H |
| 39210 | 1 | H | H | m-D7 | H | H | H | A-12 | H | H | H |
| 39211 | 1 | H | H | m-D7 | H | H | H | A-13 | H | H | H |
| 39212 | 1 | H | H | m-D7 | H | H | H | A-14 | H | H | H |
| 39213 | 1 | H | H | m-D7 | H | H | H | A-15 | H | H | H |
| 39214 | 1 | H | H | m-D7 | H | H | H | A-16 | H | H | H |
| 39215 | 1 | H | H | m-D7 | H | H | H | A-17 | H | H | H |
| 39216 | 1 | H | H | m-D7 | H | H | H | A-18 | H | H | H |
| 39217 | 1 | H | H | m-D7 | H | H | H | A-19 | H | H | H |
| 39218 | 1 | H | H | m-D7 | H | H | H | A-20 | H | H | H |
| 39219 | 1 | H | H | m-D7 | H | H | H | A-21 | H | H | H |
| 39220 | 1 | H | H | m-D7 | H | H | H | A-22 | H | H | H |
| 39221 | 1 | H | H | m-D7 | H | H | H | A-23 | H | H | H |
| 39222 | 1 | H | H | m-D7 | H | H | H | A-24 | H | H | H |
| 39223 | 1 | H | H | m-D7 | H | H | H | A-25 | H | H | H |
| 39224 | 1 | H | H | m-D7 | H | H | H | A-26 | H | H | H |
| 39225 | 1 | H | H | m-D7 | H | H | H | A-27 | H | H | H |
| 39226 | 1 | H | H | m-D7 | H | H | H | A-28 | H | H | H |
| 39227 | 1 | H | H | m-D7 | H | H | H | A-29 | H | H | H |
| 39228 | 1 | H | H | m-D7 | H | H | H | A-30 | H | H | H |
| 39229 | 1 | H | H | m-D7 | H | H | H | A-31 | H | H | H |
| 39230 | 1 | H | H | m-D7 | H | H | H | A-32 | H | H | H |
| 39231 | 1 | H | H | m-D7 | H | H | H | A-33 | H | H | H |
| 39232 | 1 | H | H | m-D7 | H | H | H | A-34 | H | H | H |
| 39233 | 1 | H | H | m-D7 | H | H | H | A-35 | H | H | H |
| 39234 | 1 | H | H | m-D7 | H | H | H | A-36 | H | H | H |
| 39235 | 1 | H | H | m-D7 | H | H | H | A-37 | H | H | H |
| 39236 | 1 | H | H | m-D7 | H | H | H | A-38 | H | H | H |
| 39237 | 1 | H | H | m-D7 | H | H | H | A-39 | H | H | H |
| 39238 | 1 | H | H | m-D7 | H | H | H | A-40 | H | H | H |
| 39239 | 1 | H | H | m-D7 | H | H | H | A-41 | H | H | H |
| 39240 | 1 | H | H | m-D7 | H | H | H | A-42 | H | H | H |
| 39241 | 1 | H | H | m-D7 | H | H | H | A-43 | H | H | H |
| 39242 | 1 | H | H | m-D7 | H | H | H | A-44 | H | H | H |
| 39243 | 1 | H | H | m-D7 | H | H | H | A-45 | H | H | H |
| 39244 | 1 | H | H | m-D7 | H | H | H | A-46 | H | H | H |
| 39245 | 1 | H | H | m-D7 | H | H | H | A-47 | H | H | H |
| 39246 | 1 | H | H | m-D8 | H | H | H | A-1 | H | H | H |
| 39247 | 1 | H | H | m-D8 | H | H | H | A-2 | H | H | H |
| 39248 | 1 | H | H | m-D8 | H | H | H | A-3 | H | H | H |
| 39249 | 1 | H | H | m-D8 | H | H | H | A-4 | H | H | H |
| 39250 | 1 | H | H | m-D8 | H | H | H | A-5 | H | H | H |
| 39251 | 1 | H | H | m-D8 | H | H | H | A-6 | H | H | H |
| 39252 | 1 | H | H | m-D8 | H | H | H | A-7 | H | H | H |
| 39253 | 1 | H | H | m-D8 | H | H | H | A-8 | H | H | H |
| 39254 | 1 | H | H | m-D8 | H | H | H | A-9 | H | H | H |
| 39255 | 1 | H | H | m-D8 | H | H | H | A-10 | H | H | H |
| 39256 | 1 | H | H | m-D8 | H | H | H | A-11 | H | H | H |
| 39257 | 1 | H | H | m-D8 | H | H | H | A-12 | H | H | H |
| 39258 | 1 | H | H | m-D8 | H | H | H | A-13 | H | H | H |
| 39259 | 1 | H | H | m-D8 | H | H | H | A-14 | H | H | H |
| 39260 | 1 | H | H | m-D8 | H | H | H | A-15 | H | H | H |
| 39261 | 1 | H | H | m-D8 | H | H | H | A-16 | H | H | H |
| 39262 | 1 | H | H | m-D8 | H | H | H | A-17 | H | H | H |
| 39263 | 1 | H | H | m-D8 | H | H | H | A-18 | H | H | H |
| 39264 | 1 | H | H | m-D8 | H | H | H | A-19 | H | H | H |
| 39265 | 1 | H | H | m-D8 | H | H | H | A-20 | H | H | H |
| 39266 | 1 | H | H | m-D8 | H | H | H | A-21 | H | H | H |
| 39267 | 1 | H | H | m-D8 | H | H | H | A-22 | H | H | H |
| 39268 | 1 | H | H | m-D8 | H | H | H | A-23 | H | H | H |
| 39269 | 1 | H | H | m-D8 | H | H | H | A-24 | H | H | H |
| 39270 | 1 | H | H | m-D8 | H | H | H | A-25 | H | H | H |
| 39271 | 1 | H | H | m-D8 | H | H | H | A-26 | H | H | H |
| 39272 | 1 | H | H | m-D8 | H | H | H | A-27 | H | H | H |
| 39273 | 1 | H | H | m-D8 | H | H | H | A-28 | H | H | H |
| 39274 | 1 | H | H | m-D8 | H | H | H | A-29 | H | H | H |
| 39275 | 1 | H | H | m-D8 | H | H | H | A-30 | H | H | H |
| 39276 | 1 | H | H | m-D8 | H | H | H | A-31 | H | H | H |
| 39277 | 1 | H | H | m-D8 | H | H | H | A-32 | H | H | H |
| 39278 | 1 | H | H | m-D8 | H | H | H | A-33 | H | H | H |
| 39279 | 1 | H | H | m-D8 | H | H | H | A-34 | H | H | H |
| 39280 | 1 | H | H | m-D8 | H | H | H | A-35 | H | H | H |
| 39281 | 1 | H | H | m-D8 | H | H | H | A-36 | H | H | H |
| 39282 | 1 | H | H | m-D8 | H | H | H | A-37 | H | H | H |
| 39283 | 1 | H | H | m-D8 | H | H | H | A-38 | H | H | H |
| 39284 | 1 | H | H | m-D8 | H | H | H | A-39 | H | H | H |
| 39285 | 1 | H | H | m-D8 | H | H | H | A-40 | H | H | H |
| 39286 | 1 | H | H | m-D8 | H | H | H | A-41 | H | H | H |
| 39287 | 1 | H | H | m-D8 | H | H | H | A-42 | H | H | H |
| 39288 | 1 | H | H | m-D8 | H | H | H | A-43 | H | H | H |
| 39289 | 1 | H | H | m-D8 | H | H | H | A-44 | H | H | H |
| 39290 | 1 | H | H | m-D8 | H | H | H | A-45 | H | H | H |
| 39291 | 1 | H | H | m-D8 | H | H | H | A-46 | H | H | H |
| 39292 | 1 | H | H | m-D8 | H | H | H | A-47 | H | H | H |
| 39293 | 1 | H | H | m-D9 | H | H | H | A-1 | H | H | H |
| 39294 | 1 | H | H | m-D9 | H | H | H | A-2 | H | H | H |
| 39295 | 1 | H | H | m-D9 | H | H | H | A-3 | H | H | H |
| 39296 | 1 | H | H | m-D9 | H | H | H | A-4 | H | H | H |
| 39297 | 1 | H | H | m-D9 | H | H | H | A-5 | H | H | H |
| 39298 | 1 | H | H | m-D9 | H | H | H | A-6 | H | H | H |
| 39299 | 1 | H | H | m-D9 | H | H | H | A-7 | H | H | H |
| 39300 | 1 | H | H | m-D9 | H | H | H | A-8 | H | H | H |
| 39301 | 1 | H | H | m-D9 | H | H | H | A-9 | H | H | H |
| 39302 | 1 | H | H | m-D9 | H | H | H | A-10 | H | H | H |
| 39303 | 1 | H | H | m-D9 | H | H | H | A-11 | H | H | H |
| 39304 | 1 | H | H | m-D9 | H | H | H | A-12 | H | H | H |
| 39305 | 1 | H | H | m-D9 | H | H | H | A-13 | H | H | H |
| 39306 | 1 | H | H | m-D9 | H | H | H | A-14 | H | H | H |
| 39307 | 1 | H | H | m-D9 | H | H | H | A-15 | H | H | H |
| 39308 | 1 | H | H | m-D9 | H | H | H | A-16 | H | H | H |
| 39309 | 1 | H | H | m-D9 | H | H | H | A-17 | H | H | H |
| 39310 | 1 | H | H | m-D9 | H | H | H | A-18 | H | H | H |
| 39311 | 1 | H | H | m-D9 | H | H | H | A-19 | H | H | H |
| 39312 | 1 | H | H | m-D9 | H | H | H | A-20 | H | H | H |
| 39313 | 1 | H | H | m-D9 | H | H | H | A-21 | H | H | H |
| 39314 | 1 | H | H | m-D9 | H | H | H | A-22 | H | H | H |
| 39315 | 1 | H | H | m-D9 | H | H | H | A-23 | H | H | H |
| 39316 | 1 | H | H | m-D9 | H | H | H | A-24 | H | H | H |
| 39317 | 1 | H | H | m-D9 | H | H | H | A-25 | H | H | H |
| 39318 | 1 | H | H | m-D9 | H | H | H | A-26 | H | H | H |
| 39319 | 1 | H | H | m-D9 | H | H | H | A-27 | H | H | H |
| 39320 | 1 | H | H | m-D9 | H | H | H | A-28 | H | H | H |
| 39321 | 1 | H | H | m-D9 | H | H | H | A-29 | H | H | H |
| 39322 | 1 | H | H | m-D9 | H | H | H | A-30 | H | H | H |
| 39323 | 1 | H | H | m-D9 | H | H | H | A-31 | H | H | H |
| 39324 | 1 | H | H | m-D9 | H | H | H | A-32 | H | H | H |
| 39325 | 1 | H | H | m-D9 | H | H | H | A-33 | H | H | H |
| 39326 | 1 | H | H | m-D9 | H | H | H | A-34 | H | H | H |
| 39327 | 1 | H | H | m-D9 | H | H | H | A-35 | H | H | H |
| 39328 | 1 | H | H | m-D9 | H | H | H | A-36 | H | H | H |
| 39329 | 1 | H | H | m-D9 | H | H | H | A-37 | H | H | H |
| 39330 | 1 | H | H | m-D9 | H | H | H | A-38 | H | H | H |
| 39331 | 1 | H | H | m-D9 | H | H | H | A-39 | H | H | H |
| 39332 | 1 | H | H | m-D9 | H | H | H | A-40 | H | H | H |
| 39333 | 1 | H | H | m-D9 | H | H | H | A-41 | H | H | H |
| 39334 | 1 | H | H | m-D9 | H | H | H | A-42 | H | H | H |
| 39335 | 1 | H | H | m-D9 | H | H | H | A-43 | H | H | H |
| 39336 | 1 | H | H | m-D9 | H | H | H | A-44 | H | H | H |
| 39337 | 1 | H | H | m-D9 | H | H | H | A-45 | H | H | H |
| 39338 | 1 | H | H | m-D9 | H | H | H | A-46 | H | H | H |
| 39339 | 1 | H | H | m-D9 | H | H | H | A-47 | H | H | H |
| 39340 | 1 | H | H | m-D10 | H | H | H | A-1 | H | H | H |
| 39341 | 1 | H | H | m-D10 | H | H | H | A-2 | H | H | H |
| 39342 | 1 | H | H | m-D10 | H | H | H | A-3 | H | H | H |
| 39343 | 1 | H | H | m-D10 | H | H | H | A-4 | H | H | H |
| 39344 | 1 | H | H | m-D10 | H | H | H | A-5 | H | H | H |
| 39345 | 1 | H | H | m-D10 | H | H | H | A-6 | H | H | H |
| 39346 | 1 | H | H | m-D10 | H | H | H | A-7 | H | H | H |
| 39347 | 1 | H | H | m-D10 | H | H | H | A-8 | H | H | H |
| 39348 | 1 | H | H | m-D10 | H | H | H | A-9 | H | H | H |
| 39349 | 1 | H | H | m-D10 | H | H | H | A-10 | H | H | H |
| 39350 | 1 | H | H | m-D10 | H | H | H | A-11 | H | H | H |
| 39351 | 1 | H | H | m-D10 | H | H | H | A-12 | H | H | H |
| 39352 | 1 | H | H | m-D10 | H | H | H | A-13 | H | H | H |
| 39353 | 1 | H | H | m-D10 | H | H | H | A-14 | H | H | H |
| 39354 | 1 | H | H | m-D10 | H | H | H | A-15 | H | H | H |
| 39355 | 1 | H | H | m-D10 | H | H | H | A-16 | H | H | H |
| 39356 | 1 | H | H | m-D10 | H | H | H | A-17 | H | H | H |
| 39357 | 1 | H | H | m-D10 | H | H | H | A-18 | H | H | H |
| 39358 | 1 | H | H | m-D10 | H | H | H | A-19 | H | H | H |
| 39359 | 1 | H | H | m-D10 | H | H | H | A-20 | H | H | H |
| 39360 | 1 | H | H | m-D10 | H | H | H | A-21 | H | H | H |
| 39361 | 1 | H | H | m-D10 | H | H | H | A-22 | H | H | H |
| 39362 | 1 | H | H | m-D10 | H | H | H | A-23 | H | H | H |
| 39363 | 1 | H | H | m-D10 | H | H | H | A-24 | H | H | H |
| 39364 | 1 | H | H | m-D10 | H | H | H | A-25 | H | H | H |
| 39365 | 1 | H | H | m-D10 | H | H | H | A-26 | H | H | H |
| 39366 | 1 | H | H | m-D10 | H | H | H | A-27 | H | H | H |
| 39367 | 1 | H | H | m-D10 | H | H | H | A-28 | H | H | H |
| 39368 | 1 | H | H | m-D10 | H | H | H | A-29 | H | H | H |
| 39369 | 1 | H | H | m-D10 | H | H | H | A-30 | H | H | H |
| 39370 | 1 | H | H | m-D10 | H | H | H | A-31 | H | H | H |
| 39371 | 1 | H | H | m-D10 | H | H | H | A-32 | H | H | H |
| 39372 | 1 | H | H | m-D10 | H | H | H | A-33 | H | H | H |
| 39373 | 1 | H | H | m-D10 | H | H | H | A-34 | H | H | H |
| 39374 | 1 | H | H | m-D10 | H | H | H | A-35 | H | H | H |
| 39375 | 1 | H | H | m-D10 | H | H | H | A-36 | H | H | H |
| 39376 | 1 | H | H | m-D10 | H | H | H | A-37 | H | H | H |
| 39377 | 1 | H | H | m-D10 | H | H | H | A-38 | H | H | H |
| 39378 | 1 | H | H | m-D10 | H | H | H | A-39 | H | H | H |
| 39379 | 1 | H | H | m-D10 | H | H | H | A-40 | H | H | H |
| 39380 | 1 | H | H | m-D10 | H | H | H | A-41 | H | H | H |
| 39381 | 1 | H | H | m-D10 | H | H | H | A-42 | H | H | H |
| 39382 | 1 | H | H | m-D10 | H | H | H | A-43 | H | H | H |
| 39383 | 1 | H | H | m-D10 | H | H | H | A-44 | H | H | H |
| 39384 | 1 | H | H | m-D10 | H | H | H | A-45 | H | H | H |
| 39385 | 1 | H | H | m-D10 | H | H | H | A-46 | H | H | H |
| 39386 | 1 | H | H | m-D10 | H | H | H | A-47 | H | H | H |
| 39387 | 1 | H | H | m-D11 | H | H | H | A-1 | H | H | H |
| 39388 | 1 | H | H | m-D11 | H | H | H | A-2 | H | H | H |
| 39389 | 1 | H | H | m-D11 | H | H | H | A-3 | H | H | H |
| 39390 | 1 | H | H | m-D11 | H | H | H | A-4 | H | H | H |
| 39391 | 1 | H | H | m-D11 | H | H | H | A-5 | H | H | H |
| 39392 | 1 | H | H | m-D11 | H | H | H | A-6 | H | H | H |
| 39393 | 1 | H | H | m-D11 | H | H | H | A-7 | H | H | H |
| 39394 | 1 | H | H | m-D11 | H | H | H | A-8 | H | H | H |
| 39395 | 1 | H | H | m-D11 | H | H | H | A-9 | H | H | H |
| 39396 | 1 | H | H | m-D11 | H | H | H | A-10 | H | H | H |
| 39397 | 1 | H | H | m-D11 | H | H | H | A-11 | H | H | H |
| 39398 | 1 | H | H | m-D11 | H | H | H | A-12 | H | H | H |
| 39399 | 1 | H | H | m-D11 | H | H | H | A-13 | H | H | H |
| 39400 | 1 | H | H | m-D11 | H | H | H | A-14 | H | H | H |
| 39401 | 1 | H | H | m-D11 | H | H | H | A-15 | H | H | H |
| 39402 | 1 | H | H | m-D11 | H | H | H | A-16 | H | H | H |
| 39403 | 1 | H | H | m-D11 | H | H | H | A-17 | H | H | H |
| 39404 | 1 | H | H | m-D11 | H | H | H | A-18 | H | H | H |
| 39405 | 1 | H | H | m-D11 | H | H | H | A-19 | H | H | H |
| 39406 | 1 | H | H | m-D11 | H | H | H | A-20 | H | H | H |
| 39407 | 1 | H | H | m-D11 | H | H | H | A-21 | H | H | H |
| 39408 | 1 | H | H | m-D11 | H | H | H | A-22 | H | H | H |
| 39409 | 1 | H | H | m-D11 | H | H | H | A-23 | H | H | H |
| 39410 | 1 | H | H | m-D11 | H | H | H | A-24 | H | H | H |
| 39411 | 1 | H | H | m-D11 | H | H | H | A-25 | H | H | H |
| 39412 | 1 | H | H | m-D11 | H | H | H | A-26 | H | H | H |
| 39413 | 1 | H | H | m-D11 | H | H | H | A-27 | H | H | H |
| 39414 | 1 | H | H | m-D11 | H | H | H | A-28 | H | H | H |
| 39415 | 1 | H | H | m-D11 | H | H | H | A-29 | H | H | H |
| 39416 | 1 | H | H | m-D11 | H | H | H | A-30 | H | H | H |
| 39417 | 1 | H | H | m-D11 | H | H | H | A-31 | H | H | H |
| 39418 | 1 | H | H | m-D11 | H | H | H | A-32 | H | H | H |
| 39419 | 1 | H | H | m-D11 | H | H | H | A-33 | H | H | H |
| 39420 | 1 | H | H | m-D11 | H | H | H | A-34 | H | H | H |
| 39421 | 1 | H | H | m-D11 | H | H | H | A-35 | H | H | H |
| 39422 | 1 | H | H | m-D11 | H | H | H | A-36 | H | H | H |
| 39423 | 1 | H | H | m-D11 | H | H | H | A-37 | H | H | H |
| 39424 | 1 | H | H | m-D11 | H | H | H | A-38 | H | H | H |
| 39425 | 1 | H | H | m-D11 | H | H | H | A-39 | H | H | H |
| 39426 | 1 | H | H | m-D11 | H | H | H | A-40 | H | H | H |
| 39427 | 1 | H | H | m-D11 | H | H | H | A-41 | H | H | H |
| 39428 | 1 | H | H | m-D11 | H | H | H | A-42 | H | H | H |
| 39429 | 1 | H | H | m-D11 | H | H | H | A-43 | H | H | H |
| 39430 | 1 | H | H | m-D11 | H | H | H | A-44 | H | H | H |
| 39431 | 1 | H | H | m-D11 | H | H | H | A-45 | H | H | H |
| 39432 | 1 | H | H | m-D11 | H | H | H | A-46 | H | H | H |
| 39433 | 1 | H | H | m-D11 | H | H | H | A-47 | H | H | H |
| 39434 | 1 | H | H | m-D12 | H | H | H | A-1 | H | H | H |
| 39435 | 1 | H | H | m-D12 | H | H | H | A-2 | H | H | H |
| 39436 | 1 | H | H | m-D12 | H | H | H | A-3 | H | H | H |
| 39437 | 1 | H | H | m-D12 | H | H | H | A-4 | H | H | H |
| 39438 | 1 | H | H | m-D12 | H | H | H | A-5 | H | H | H |
| 39439 | 1 | H | H | m-D12 | H | H | H | A-6 | H | H | H |
| 39440 | 1 | H | H | m-D12 | H | H | H | A-7 | H | H | H |
| 39441 | 1 | H | H | m-D12 | H | H | H | A-8 | H | H | H |
| 39442 | 1 | H | H | m-D12 | H | H | H | A-9 | H | H | H |
| 39443 | 1 | H | H | m-D12 | H | H | H | A-10 | H | H | H |
| 39444 | 1 | H | H | m-D12 | H | H | H | A-11 | H | H | H |
| 39445 | 1 | H | H | m-D12 | H | H | H | A-12 | H | H | H |
| 39446 | 1 | H | H | m-D12 | H | H | H | A-13 | H | H | H |
| 39447 | 1 | H | H | m-D12 | H | H | H | A-14 | H | H | H |
| 39448 | 1 | H | H | m-D12 | H | H | H | A-15 | H | H | H |
| 39449 | 1 | H | H | m-D12 | H | H | H | A-16 | H | H | H |
| 39450 | 1 | H | H | m-D12 | H | H | H | A-17 | H | H | H |
| 39451 | 1 | H | H | m-D12 | H | H | H | A-18 | H | H | H |
| 39452 | 1 | H | H | m-D12 | H | H | H | A-19 | H | H | H |
| 39453 | 1 | H | H | m-D12 | H | H | H | A-20 | H | H | H |
| 39454 | 1 | H | H | m-D12 | H | H | H | A-21 | H | H | H |
| 39455 | 1 | H | H | m-D12 | H | H | H | A-22 | H | H | H |
| 39456 | 1 | H | H | m-D12 | H | H | H | A-23 | H | H | H |
| 39457 | 1 | H | H | m-D12 | H | H | H | A-24 | H | H | H |
| 39458 | 1 | H | H | m-D12 | H | H | H | A-25 | H | H | H |
| 39459 | 1 | H | H | m-D12 | H | H | H | A-26 | H | H | H |
| 39460 | 1 | H | H | m-D12 | H | H | H | A-27 | H | H | H |
| 39461 | 1 | H | H | m-D12 | H | H | H | A-28 | H | H | H |
| 39462 | 1 | H | H | m-D12 | H | H | H | A-29 | H | H | H |
| 39463 | 1 | H | H | m-D12 | H | H | H | A-30 | H | H | H |
| 39464 | 1 | H | H | m-D12 | H | H | H | A-31 | H | H | H |
| 39465 | 1 | H | H | m-D12 | H | H | H | A-32 | H | H | H |
| 39466 | 1 | H | H | m-D12 | H | H | H | A-33 | H | H | H |
| 39467 | 1 | H | H | m-D12 | H | H | H | A-34 | H | H | H |
| 39468 | 1 | H | H | m-D12 | H | H | H | A-35 | H | H | H |
| 39469 | 1 | H | H | m-D12 | H | H | H | A-36 | H | H | H |
| 39470 | 1 | H | H | m-D12 | H | H | H | A-37 | H | H | H |
| 39471 | 1 | H | H | m-D12 | H | H | H | A-38 | H | H | H |
| 39472 | 1 | H | H | m-D12 | H | H | H | A-39 | H | H | H |
| 39473 | 1 | H | H | m-D12 | H | H | H | A-40 | H | H | H |
| 39474 | 1 | H | H | m-D12 | H | H | H | A-41 | H | H | H |
| 39475 | 1 | H | H | m-D12 | H | H | H | A-42 | H | H | H |
| 39476 | 1 | H | H | m-D12 | H | H | H | A-43 | H | H | H |
| 39477 | 1 | H | H | m-D12 | H | H | H | A-44 | H | H | H |
| 39478 | 1 | H | H | m-D12 | H | H | H | A-45 | H | H | H |
| 39479 | 1 | H | H | m-D12 | H | H | H | A-46 | H | H | H |
| 39480 | 1 | H | H | m-D12 | H | H | H | A-47 | H | H | H |
| 39481 | 1 | H | H | m-D13 | H | H | H | A-1 | H | H | H |
| 39482 | 1 | H | H | m-D13 | H | H | H | A-2 | H | H | H |
| 39483 | 1 | H | H | m-D13 | H | H | H | A-3 | H | H | H |
| 39484 | 1 | H | H | m-D13 | H | H | H | A-4 | H | H | H |
| 39485 | 1 | H | H | m-D13 | H | H | H | A-5 | H | H | H |
| 39486 | 1 | H | H | m-D13 | H | H | H | A-6 | H | H | H |
| 39487 | 1 | H | H | m-D13 | H | H | H | A-7 | H | H | H |
| 39488 | 1 | H | H | m-D13 | H | H | H | A-8 | H | H | H |
| 39489 | 1 | H | H | m-D13 | H | H | H | A-9 | H | H | H |
| 39490 | 1 | H | H | m-D13 | H | H | H | A-10 | H | H | H |
| 39491 | 1 | H | H | m-D13 | H | H | H | A-11 | H | H | H |
| 39492 | 1 | H | H | m-D13 | H | H | H | A-12 | H | H | H |
| 39493 | 1 | H | H | m-D13 | H | H | H | A-13 | H | H | H |
| 39494 | 1 | H | H | m-D13 | H | H | H | A-14 | H | H | H |
| 39495 | 1 | H | H | m-D13 | H | H | H | A-15 | H | H | H |
| 39496 | 1 | H | H | m-D13 | H | H | H | A-16 | H | H | H |
| 39497 | 1 | H | H | m-D13 | H | H | H | A-17 | H | H | H |
| 39498 | 1 | H | H | m-D13 | H | H | H | A-18 | H | H | H |
| 39499 | 1 | H | H | m-D13 | H | H | H | A-19 | H | H | H |
| 39500 | 1 | H | H | m-D13 | H | H | H | A-20 | H | H | H |
| 39501 | 1 | H | H | m-D13 | H | H | H | A-21 | H | H | H |
| 39502 | 1 | H | H | m-D13 | H | H | H | A-22 | H | H | H |
| 39503 | 1 | H | H | m-D13 | H | H | H | A-23 | H | H | H |
| 39504 | 1 | H | H | m-D13 | H | H | H | A-24 | H | H | H |
| 39505 | 1 | H | H | m-D13 | H | H | H | A-25 | H | H | H |
| 39506 | 1 | H | H | m-D13 | H | H | H | A-26 | H | H | H |
| 39507 | 1 | H | H | m-D13 | H | H | H | A-27 | H | H | H |
| 39508 | 1 | H | H | m-D13 | H | H | H | A-28 | H | H | H |
| 39509 | 1 | H | H | m-D13 | H | H | H | A-29 | H | H | H |
| 39510 | 1 | H | H | m-D13 | H | H | H | A-30 | H | H | H |
| 39511 | 1 | H | H | m-D13 | H | H | H | A-31 | H | H | H |
| 39512 | 1 | H | H | m-D13 | H | H | H | A-32 | H | H | H |
| 39513 | 1 | H | H | m-D13 | H | H | H | A-33 | H | H | H |
| 39514 | 1 | H | H | m-D13 | H | H | H | A-34 | H | H | H |
| 39515 | 1 | H | H | m-D13 | H | H | H | A-35 | H | H | H |
| 39516 | 1 | H | H | m-D13 | H | H | H | A-36 | H | H | H |
| 39517 | 1 | H | H | m-D13 | H | H | H | A-37 | H | H | H |
| 39518 | 1 | H | H | m-D13 | H | H | H | A-38 | H | H | H |
| 39519 | 1 | H | H | m-D13 | H | H | H | A-39 | H | H | H |
| 39520 | 1 | H | H | m-D13 | H | H | H | A-40 | H | H | H |
| 39521 | 1 | H | H | m-D13 | H | H | H | A-41 | H | H | H |
| 39522 | 1 | H | H | m-D13 | H | H | H | A-42 | H | H | H |
| 39523 | 1 | H | H | m-D13 | H | H | H | A-43 | H | H | H |
| 39524 | 1 | H | H | m-D13 | H | H | H | A-44 | H | H | H |
| 39525 | 1 | H | H | m-D13 | H | H | H | A-45 | H | H | H |
| 39526 | 1 | H | H | m-D13 | H | H | H | A-46 | H | H | H |
| 39527 | 1 | H | H | m-D13 | H | H | H | A-47 | H | H | H |
| 39528 | 1 | H | H | m-D14 | H | H | H | A-1 | H | H | H |
| 39529 | 1 | H | H | m-D14 | H | H | H | A-2 | H | H | H |
| 39530 | 1 | H | H | m-D14 | H | H | H | A-3 | H | H | H |
| 39531 | 1 | H | H | m-D14 | H | H | H | A-4 | H | H | H |
| 39532 | 1 | H | H | m-D14 | H | H | H | A-5 | H | H | H |
| 39533 | 1 | H | H | m-D14 | H | H | H | A-6 | H | H | H |
| 39534 | 1 | H | H | m-D14 | H | H | H | A-7 | H | H | H |
| 39535 | 1 | H | H | m-D14 | H | H | H | A-8 | H | H | H |
| 39536 | 1 | H | H | m-D14 | H | H | H | A-9 | H | H | H |
| 39537 | 1 | H | H | m-D14 | H | H | H | A-10 | H | H | H |
| 39538 | 1 | H | H | m-D14 | H | H | H | A-11 | H | H | H |
| 39539 | 1 | H | H | m-D14 | H | H | H | A-12 | H | H | H |
| 39540 | 1 | H | H | m-D14 | H | H | H | A-13 | H | H | H |
| 39541 | 1 | H | H | m-D14 | H | H | H | A-14 | H | H | H |
| 39542 | 1 | H | H | m-D14 | H | H | H | A-15 | H | H | H |
| 39543 | 1 | H | H | m-D14 | H | H | H | A-16 | H | H | H |
| 39544 | 1 | H | H | m-D14 | H | H | H | A-17 | H | H | H |
| 39545 | 1 | H | H | m-D14 | H | H | H | A-18 | H | H | H |
| 39546 | 1 | H | H | m-D14 | H | H | H | A-19 | H | H | H |
| 39547 | 1 | H | H | m-D14 | H | H | H | A-20 | H | H | H |
| 39548 | 1 | H | H | m-D14 | H | H | H | A-21 | H | H | H |
| 39549 | 1 | H | H | m-D14 | H | H | H | A-22 | H | H | H |
| 39550 | 1 | H | H | m-D14 | H | H | H | A-23 | H | H | H |
| 39551 | 1 | H | H | m-D14 | H | H | H | A-24 | H | H | H |
| 39552 | 1 | H | H | m-D14 | H | H | H | A-25 | H | H | H |
| 39553 | 1 | H | H | m-D14 | H | H | H | A-26 | H | H | H |
| 39554 | 1 | H | H | m-D14 | H | H | H | A-27 | H | H | H |
| 39555 | 1 | H | H | m-D14 | H | H | H | A-28 | H | H | H |
| 39556 | 1 | H | H | m-D14 | H | H | H | A-29 | H | H | H |
| 39557 | 1 | H | H | m-D14 | H | H | H | A-30 | H | H | H |
| 39558 | 1 | H | H | m-D14 | H | H | H | A-31 | H | H | H |
| 39559 | 1 | H | H | m-D14 | H | H | H | A-32 | H | H | H |
| 39560 | 1 | H | H | m-D14 | H | H | H | A-33 | H | H | H |
| 39561 | 1 | H | H | m-D14 | H | H | H | A-34 | H | H | H |
| 39562 | 1 | H | H | m-D14 | H | H | H | A-35 | H | H | H |
| 39563 | 1 | H | H | m-D14 | H | H | H | A-36 | H | H | H |
| 39564 | 1 | H | H | m-D14 | H | H | H | A-37 | H | H | H |
| 39565 | 1 | H | H | m-D14 | H | H | H | A-38 | H | H | H |
| 39566 | 1 | H | H | m-D14 | H | H | H | A-39 | H | H | H |
| 39567 | 1 | H | H | m-D14 | H | H | H | A-40 | H | H | H |
| 39568 | 1 | H | H | m-D14 | H | H | H | A-41 | H | H | H |
| 39569 | 1 | H | H | m-D14 | H | H | H | A-42 | H | H | H |
| 39570 | 1 | H | H | m-D14 | H | H | H | A-43 | H | H | H |
| 39571 | 1 | H | H | m-D14 | H | H | H | A-44 | H | H | H |
| 39572 | 1 | H | H | m-D14 | H | H | H | A-45 | H | H | H |
| 39573 | 1 | H | H | m-D14 | H | H | H | A-46 | H | H | H |
| 39574 | 1 | H | H | m-D14 | H | H | H | A-47 | H | H | H |
| 39575 | 1 | H | H | A-1 | H | H | H | m-D1 | H | H | H |
| 39576 | 1 | H | H | A-2 | H | H | H | m-D1 | H | H | H |
| 39577 | 1 | H | H | A-3 | H | H | H | m-D1 | H | H | H |
| 39578 | 1 | H | H | A-4 | H | H | H | m-D1 | H | H | H |
| 39579 | 1 | H | H | A-5 | H | H | H | m-D1 | H | H | H |
| 39580 | 1 | H | H | A-6 | H | H | H | m-D1 | H | H | H |
| 39581 | 1 | H | H | A-7 | H | H | H | m-D1 | H | H | H |
| 39582 | 1 | H | H | A-8 | H | H | H | m-D1 | H | H | H |
| 39583 | 1 | H | H | A-9 | H | H | H | m-D1 | H | H | H |
| 39584 | 1 | H | H | A-10 | H | H | H | m-D1 | H | H | H |
| 39585 | 1 | H | H | A-11 | H | H | H | m-D1 | H | H | H |
| 39586 | 1 | H | H | A-12 | H | H | H | m-D1 | H | H | H |
| 39587 | 1 | H | H | A-13 | H | H | H | m-D1 | H | H | H |
| 39588 | 1 | H | H | A-14 | H | H | H | m-D1 | H | H | H |
| 39589 | 1 | H | H | A-15 | H | H | H | m-D1 | H | H | H |
| 39590 | 1 | H | H | A-16 | H | H | H | m-D1 | H | H | H |
| 39591 | 1 | H | H | A-17 | H | H | H | m-D1 | H | H | H |
| 39592 | 1 | H | H | A-18 | H | H | H | m-D1 | H | H | H |
| 39593 | 1 | H | H | A-19 | H | H | H | m-D1 | H | H | H |
| 39594 | 1 | H | H | A-20 | H | H | H | m-D1 | H | H | H |
| 39595 | 1 | H | H | A-21 | H | H | H | m-D1 | H | H | H |
| 39596 | 1 | H | H | A-22 | H | H | H | m-D1 | H | H | H |
| 39597 | 1 | H | H | A-23 | H | H | H | m-D1 | H | H | H |
| 39598 | 1 | H | H | A-24 | H | H | H | m-D1 | H | H | H |
| 39599 | 1 | H | H | A-25 | H | H | H | m-D1 | H | H | H |
| 39600 | 1 | H | H | A-26 | H | H | H | m-D1 | H | H | H |
| 39601 | 1 | H | H | A-27 | H | H | H | m-D1 | H | H | H |
| 39602 | 1 | H | H | A-28 | H | H | H | m-D1 | H | H | H |
| 39603 | 1 | H | H | A-29 | H | H | H | m-D1 | H | H | H |
| 39604 | 1 | H | H | A-30 | H | H | H | m-D1 | H | H | H |
| 39605 | 1 | H | H | A-31 | H | H | H | m-D1 | H | H | H |
| 39606 | 1 | H | H | A-32 | H | H | H | m-D1 | H | H | H |
| 39607 | 1 | H | H | A-33 | H | H | H | m-D1 | H | H | H |
| 39608 | 1 | H | H | A-34 | H | H | H | m-D1 | H | H | H |
| 39609 | 1 | H | H | A-35 | H | H | H | m-D1 | H | H | H |
| 39610 | 1 | H | H | A-36 | H | H | H | m-D1 | H | H | H |
| 39611 | 1 | H | H | A-37 | H | H | H | m-D1 | H | H | H |
| 39612 | 1 | H | H | A-38 | H | H | H | m-D1 | H | H | H |
| 39613 | 1 | H | H | A-39 | H | H | H | m-D1 | H | H | H |
| 39614 | 1 | H | H | A-40 | H | H | H | m-D1 | H | H | H |
| 39615 | 1 | H | H | A-41 | H | H | H | m-D1 | H | H | H |
| 39616 | 1 | H | H | A-42 | H | H | H | m-D1 | H | H | H |
| 39617 | 1 | H | H | A-43 | H | H | H | m-D1 | H | H | H |
| 39618 | 1 | H | H | A-44 | H | H | H | m-D1 | H | H | H |
| 39619 | 1 | H | H | A-45 | H | H | H | m-D1 | H | H | H |
| 39620 | 1 | H | H | A-46 | H | H | H | m-D1 | H | H | H |
| 39621 | 1 | H | H | A-47 | H | H | H | m-D1 | H | H | H |
| 39622 | 1 | H | H | A-1 | H | H | H | m-D2 | H | H | H |
| 39623 | 1 | H | H | A-2 | H | H | H | m-D2 | H | H | H |
| 39624 | 1 | H | H | A-3 | H | H | H | m-D2 | H | H | H |
| 39625 | 1 | H | H | A-4 | H | H | H | m-D2 | H | H | H |
| 39626 | 1 | H | H | A-5 | H | H | H | m-D2 | H | H | H |
| 39627 | 1 | H | H | A-6 | H | H | H | m-D2 | H | H | H |
| 39628 | 1 | H | H | A-7 | H | H | H | m-D2 | H | H | H |
| 39629 | 1 | H | H | A-8 | H | H | H | m-D2 | H | H | H |
| 39630 | 1 | H | H | A-9 | H | H | H | m-D2 | H | H | H |
| 39631 | 1 | H | H | A-10 | H | H | H | m-D2 | H | H | H |
| 39632 | 1 | H | H | A-11 | H | H | H | m-D2 | H | H | H |
| 39633 | 1 | H | H | A-12 | H | H | H | m-D2 | H | H | H |
| 39634 | 1 | H | H | A-13 | H | H | H | m-D2 | H | H | H |
| 39635 | 1 | H | H | A-14 | H | H | H | m-D2 | H | H | H |
| 39636 | 1 | H | H | A-15 | H | H | H | m-D2 | H | H | H |
| 39637 | 1 | H | H | A-16 | H | H | H | m-D2 | H | H | H |
| 39638 | 1 | H | H | A-17 | H | H | H | m-D2 | H | H | H |
| 39639 | 1 | H | H | A-18 | H | H | H | m-D2 | H | H | H |
| 39640 | 1 | H | H | A-19 | H | H | H | m-D2 | H | H | H |
| 39641 | 1 | H | H | A-20 | H | H | H | m-D2 | H | H | H |
| 39642 | 1 | H | H | A-21 | H | H | H | m-D2 | H | H | H |
| 39643 | 1 | H | H | A-22 | H | H | H | m-D2 | H | H | H |
| 39644 | 1 | H | H | A-23 | H | H | H | m-D2 | H | H | H |
| 39645 | 1 | H | H | A-24 | H | H | H | m-D2 | H | H | H |
| 39646 | 1 | H | H | A-25 | H | H | H | m-D2 | H | H | H |
| 39647 | 1 | H | H | A-26 | H | H | H | m-D2 | H | H | H |
| 39648 | 1 | H | H | A-27 | H | H | H | m-D2 | H | H | H |
| 39649 | 1 | H | H | A-28 | H | H | H | m-D2 | H | H | H |
| 39650 | 1 | H | H | A-29 | H | H | H | m-D2 | H | H | H |
| 39651 | 1 | H | H | A-30 | H | H | H | m-D2 | H | H | H |
| 39652 | 1 | H | H | A-31 | H | H | H | m-D2 | H | H | H |
| 39653 | 1 | H | H | A-32 | H | H | H | m-D2 | H | H | H |
| 39654 | 1 | H | H | A-33 | H | H | H | m-D2 | H | H | H |
| 39655 | 1 | H | H | A-34 | H | H | H | m-D2 | H | H | H |
| 39656 | 1 | H | H | A-35 | H | H | H | m-D2 | H | H | H |
| 39657 | 1 | H | H | A-36 | H | H | H | m-D2 | H | H | H |
| 39658 | 1 | H | H | A-37 | H | H | H | m-D2 | H | H | H |
| 39659 | 1 | H | H | A-38 | H | H | H | m-D2 | H | H | H |
| 39660 | 1 | H | H | A-39 | H | H | H | m-D2 | H | H | H |
| 39661 | 1 | H | H | A-40 | H | H | H | m-D2 | H | H | H |
| 39662 | 1 | H | H | A-41 | H | H | H | m-D2 | H | H | H |
| 39663 | 1 | H | H | A-42 | H | H | H | m-D2 | H | H | H |
| 39664 | 1 | H | H | A-43 | H | H | H | m-D2 | H | H | H |
| 39665 | 1 | H | H | A-44 | H | H | H | m-D2 | H | H | H |
| 39666 | 1 | H | H | A-45 | H | H | H | m-D2 | H | H | H |
| 39667 | 1 | H | H | A-46 | H | H | H | m-D2 | H | H | H |
| 39668 | 1 | H | H | A-47 | H | H | H | m-D2 | H | H | H |
| 39669 | 1 | H | H | A-1 | H | H | H | m-D3 | H | H | H |
| 39670 | 1 | H | H | A-2 | H | H | H | m-D3 | H | H | H |
| 39671 | 1 | H | H | A-3 | H | H | H | m-D3 | H | H | H |
| 39672 | 1 | H | H | A-4 | H | H | H | m-D3 | H | H | H |
| 39673 | 1 | H | H | A-5 | H | H | H | m-D3 | H | H | H |
| 39674 | 1 | H | H | A-6 | H | H | H | m-D3 | H | H | H |
| 39675 | 1 | H | H | A-7 | H | H | H | m-D3 | H | H | H |
| 39676 | 1 | H | H | A-8 | H | H | H | m-D3 | H | H | H |
| 39677 | 1 | H | H | A-9 | H | H | H | m-D3 | H | H | H |
| 39678 | 1 | H | H | A-10 | H | H | H | m-D3 | H | H | H |
| 39679 | 1 | H | H | A-11 | H | H | H | m-D3 | H | H | H |
| 39680 | 1 | H | H | A-12 | H | H | H | m-D3 | H | H | H |
| 39681 | 1 | H | H | A-13 | H | H | H | m-D3 | H | H | H |
| 39682 | 1 | H | H | A-14 | H | H | H | m-D3 | H | H | H |
| 39683 | 1 | H | H | A-15 | H | H | H | m-D3 | H | H | H |
| 39684 | 1 | H | H | A-16 | H | H | H | m-D3 | H | H | H |
| 39685 | 1 | H | H | A-17 | H | H | H | m-D3 | H | H | H |
| 39686 | 1 | H | H | A-18 | H | H | H | m-D3 | H | H | H |
| 39687 | 1 | H | H | A-19 | H | H | H | m-D3 | H | H | H |
| 39688 | 1 | H | H | A-20 | H | H | H | m-D3 | H | H | H |
| 39689 | 1 | H | H | A-21 | H | H | H | m-D3 | H | H | H |
| 39690 | 1 | H | H | A-22 | H | H | H | m-D3 | H | H | H |
| 39691 | 1 | H | H | A-23 | H | H | H | m-D3 | H | H | H |
| 39692 | 1 | H | H | A-24 | H | H | H | m-D3 | H | H | H |
| 39693 | 1 | H | H | A-25 | H | H | H | m-D3 | H | H | H |
| 39694 | 1 | H | H | A-26 | H | H | H | m-D3 | H | H | H |
| 39695 | 1 | H | H | A-27 | H | H | H | m-D3 | H | H | H |
| 39696 | 1 | H | H | A-28 | H | H | H | m-D3 | H | H | H |
| 39697 | 1 | H | H | A-29 | H | H | H | m-D3 | H | H | H |
| 39698 | 1 | H | H | A-30 | H | H | H | m-D3 | H | H | H |
| 39699 | 1 | H | H | A-31 | H | H | H | m-D3 | H | H | H |
| 39700 | 1 | H | H | A-32 | H | H | H | m-D3 | H | H | H |
| 39701 | 1 | H | H | A-33 | H | H | H | m-D3 | H | H | H |
| 39702 | 1 | H | H | A-34 | H | H | H | m-D3 | H | H | H |
| 39703 | 1 | H | H | A-35 | H | H | H | m-D3 | H | H | H |
| 39704 | 1 | H | H | A-36 | H | H | H | m-D3 | H | H | H |
| 39705 | 1 | H | H | A-37 | H | H | H | m-D3 | H | H | H |
| 39706 | 1 | H | H | A-38 | H | H | H | m-D3 | H | H | H |
| 39707 | 1 | H | H | A-39 | H | H | H | m-D3 | H | H | H |
| 39708 | 1 | H | H | A-40 | H | H | H | m-D3 | H | H | H |
| 39709 | 1 | H | H | A-41 | H | H | H | m-D3 | H | H | H |
| 39710 | 1 | H | H | A-42 | H | H | H | m-D3 | H | H | H |
| 39711 | 1 | H | H | A-43 | H | H | H | m-D3 | H | H | H |
| 39712 | 1 | H | H | A-44 | H | H | H | m-D3 | H | H | H |
| 39713 | 1 | H | H | A-45 | H | H | H | m-D3 | H | H | H |
| 39714 | 1 | H | H | A-46 | H | H | H | m-D3 | H | H | H |
| 39715 | 1 | H | H | A-47 | H | H | H | m-D3 | H | H | H |
| 39716 | 1 | H | H | A-1 | H | H | H | m-D4 | H | H | H |
| 39717 | 1 | H | H | A-2 | H | H | H | m-D4 | H | H | H |
| 39718 | 1 | H | H | A-3 | H | H | H | m-D4 | H | H | H |
| 39719 | 1 | H | H | A-4 | H | H | H | m-D4 | H | H | H |
| 39720 | 1 | H | H | A-5 | H | H | H | m-D4 | H | H | H |
| 39721 | 1 | H | H | A-6 | H | H | H | m-D4 | H | H | H |
| 39722 | 1 | H | H | A-7 | H | H | H | m-D4 | H | H | H |
| 39723 | 1 | H | H | A-8 | H | H | H | m-D4 | H | H | H |
| 39724 | 1 | H | H | A-9 | H | H | H | m-D4 | H | H | H |
| 39725 | 1 | H | H | A-10 | H | H | H | m-D4 | H | H | H |
| 39726 | 1 | H | H | A-11 | H | H | H | m-D4 | H | H | H |
| 39727 | 1 | H | H | A-12 | H | H | H | m-D4 | H | H | H |
| 39728 | 1 | H | H | A-13 | H | H | H | m-D4 | H | H | H |
| 39729 | 1 | H | H | A-14 | H | H | H | m-D4 | H | H | H |
| 39730 | 1 | H | H | A-15 | H | H | H | m-D4 | H | H | H |
| 39731 | 1 | H | H | A-16 | H | H | H | m-D4 | H | H | H |
| 39732 | 1 | H | H | A-17 | H | H | H | m-D4 | H | H | H |
| 39733 | 1 | H | H | A-18 | H | H | H | m-D4 | H | H | H |
| 39734 | 1 | H | H | A-19 | H | H | H | m-D4 | H | H | H |
| 39735 | 1 | H | H | A-20 | H | H | H | m-D4 | H | H | H |
| 39736 | 1 | H | H | A-21 | H | H | H | m-D4 | H | H | H |
| 39737 | 1 | H | H | A-22 | H | H | H | m-D4 | H | H | H |
| 39738 | 1 | H | H | A-23 | H | H | H | m-D4 | H | H | H |
| 39739 | 1 | H | H | A-24 | H | H | H | m-D4 | H | H | H |
| 39740 | 1 | H | H | A-25 | H | H | H | m-D4 | H | H | H |
| 39741 | 1 | H | H | A-26 | H | H | H | m-D4 | H | H | H |
| 39742 | 1 | H | H | A-27 | H | H | H | m-D4 | H | H | H |
| 39743 | 1 | H | H | A-28 | H | H | H | m-D4 | H | H | H |
| 39744 | 1 | H | H | A-29 | H | H | H | m-D4 | H | H | H |
| 39745 | 1 | H | H | A-30 | H | H | H | m-D4 | H | H | H |
| 39746 | 1 | H | H | A-31 | H | H | H | m-D4 | H | H | H |
| 39747 | 1 | H | H | A-32 | H | H | H | m-D4 | H | H | H |
| 39748 | 1 | H | H | A-33 | H | H | H | m-D4 | H | H | H |
| 39749 | 1 | H | H | A-34 | H | H | H | m-D4 | H | H | H |
| 39750 | 1 | H | H | A-35 | H | H | H | m-D4 | H | H | H |
| 39751 | 1 | H | H | A-36 | H | H | H | m-D4 | H | H | H |
| 39752 | 1 | H | H | A-37 | H | H | H | m-D4 | H | H | H |
| 39753 | 1 | H | H | A-38 | H | H | H | m-D4 | H | H | H |
| 39754 | 1 | H | H | A-39 | H | H | H | m-D4 | H | H | H |
| 39755 | 1 | H | H | A-40 | H | H | H | m-D4 | H | H | H |
| 39756 | 1 | H | H | A-41 | H | H | H | m-D4 | H | H | H |
| 39757 | 1 | H | H | A-42 | H | H | H | m-D4 | H | H | H |
| 39758 | 1 | H | H | A-43 | H | H | H | m-D4 | H | H | H |
| 39759 | 1 | H | H | A-44 | H | H | H | m-D4 | H | H | H |
| 39760 | 1 | H | H | A-45 | H | H | H | m-D4 | H | H | H |
| 39761 | 1 | H | H | A-46 | H | H | H | m-D4 | H | H | H |
| 39762 | 1 | H | H | A-47 | H | H | H | m-D4 | H | H | H |
| 39763 | 1 | H | H | A-1 | H | H | H | m-D5 | H | H | H |
| 39764 | 1 | H | H | A-2 | H | H | H | m-D5 | H | H | H |
| 39765 | 1 | H | H | A-3 | H | H | H | m-D5 | H | H | H |
| 39766 | 1 | H | H | A-4 | H | H | H | m-D5 | H | H | H |
| 39767 | 1 | H | H | A-5 | H | H | H | m-D5 | H | H | H |
| 39768 | 1 | H | H | A-6 | H | H | H | m-D5 | H | H | H |
| 39769 | 1 | H | H | A-7 | H | H | H | m-D5 | H | H | H |
| 39770 | 1 | H | H | A-8 | H | H | H | m-D5 | H | H | H |
| 39771 | 1 | H | H | A-9 | H | H | H | m-D5 | H | H | H |
| 39772 | 1 | H | H | A-10 | H | H | H | m-D5 | H | H | H |
| 39773 | 1 | H | H | A-11 | H | H | H | m-D5 | H | H | H |
| 39774 | 1 | H | H | A-12 | H | H | H | m-D5 | H | H | H |
| 39775 | 1 | H | H | A-13 | H | H | H | m-D5 | H | H | H |
| 39776 | 1 | H | H | A-14 | H | H | H | m-D5 | H | H | H |
| 39777 | 1 | H | H | A-15 | H | H | H | m-D5 | H | H | H |
| 39778 | 1 | H | H | A-16 | H | H | H | m-D5 | H | H | H |
| 39779 | 1 | H | H | A-17 | H | H | H | m-D5 | H | H | H |
| 39780 | 1 | H | H | A-18 | H | H | H | m-D5 | H | H | H |
| 39781 | 1 | H | H | A-19 | H | H | H | m-D5 | H | H | H |
| 39782 | 1 | H | H | A-20 | H | H | H | m-D5 | H | H | H |
| 39783 | 1 | H | H | A-21 | H | H | H | m-D5 | H | H | H |
| 39784 | 1 | H | H | A-22 | H | H | H | m-D5 | H | H | H |
| 39785 | 1 | H | H | A-23 | H | H | H | m-D5 | H | H | H |
| 39786 | 1 | H | H | A-24 | H | H | H | m-D5 | H | H | H |
| 39787 | 1 | H | H | A-25 | H | H | H | m-D5 | H | H | H |
| 39788 | 1 | H | H | A-26 | H | H | H | m-D5 | H | H | H |
| 39789 | 1 | H | H | A-27 | H | H | H | m-D5 | H | H | H |
| 39790 | 1 | H | H | A-28 | H | H | H | m-D5 | H | H | H |
| 39791 | 1 | H | H | A-29 | H | H | H | m-D5 | H | H | H |
| 39792 | 1 | H | H | A-30 | H | H | H | m-D5 | H | H | H |
| 39793 | 1 | H | H | A-31 | H | H | H | m-D5 | H | H | H |
| 39794 | 1 | H | H | A-32 | H | H | H | m-D5 | H | H | H |
| 39795 | 1 | H | H | A-33 | H | H | H | m-D5 | H | H | H |
| 39796 | 1 | H | H | A-34 | H | H | H | m-D5 | H | H | H |
| 39797 | 1 | H | H | A-35 | H | H | H | m-D5 | H | H | H |
| 39798 | 1 | H | H | A-36 | H | H | H | m-D5 | H | H | H |
| 39799 | 1 | H | H | A-37 | H | H | H | m-D5 | H | H | H |
| 39800 | 1 | H | H | A-38 | H | H | H | m-D5 | H | H | H |
| 39801 | 1 | H | H | A-39 | H | H | H | m-D5 | H | H | H |
| 39802 | 1 | H | H | A-40 | H | H | H | m-D5 | H | H | H |
| 39803 | 1 | H | H | A-41 | H | H | H | m-D5 | H | H | H |
| 39804 | 1 | H | H | A-42 | H | H | H | m-D5 | H | H | H |
| 39805 | 1 | H | H | A-43 | H | H | H | m-D5 | H | H | H |
| 39806 | 1 | H | H | A-44 | H | H | H | m-D5 | H | H | H |
| 39807 | 1 | H | H | A-45 | H | H | H | m-D5 | H | H | H |
| 39808 | 1 | H | H | A-46 | H | H | H | m-D5 | H | H | H |
| 39809 | 1 | H | H | A-47 | H | H | H | m-D5 | H | H | H |
| 39810 | 1 | H | H | A-1 | H | H | H | m-D6 | H | H | H |
| 39811 | 1 | H | H | A-2 | H | H | H | m-D6 | H | H | H |
| 39812 | 1 | H | H | A-3 | H | H | H | m-D6 | H | H | H |
| 39813 | 1 | H | H | A-4 | H | H | H | m-D6 | H | H | H |
| 39814 | 1 | H | H | A-5 | H | H | H | m-D6 | H | H | H |
| 39815 | 1 | H | H | A-6 | H | H | H | m-D6 | H | H | H |
| 39816 | 1 | H | H | A-7 | H | H | H | m-D6 | H | H | H |
| 39817 | 1 | H | H | A-8 | H | H | H | m-D6 | H | H | H |
| 39818 | 1 | H | H | A-9 | H | H | H | m-D6 | H | H | H |
| 39819 | 1 | H | H | A-10 | H | H | H | m-D6 | H | H | H |
| 39820 | 1 | H | H | A-11 | H | H | H | m-D6 | H | H | H |
| 39821 | 1 | H | H | A-12 | H | H | H | m-D6 | H | H | H |
| 39822 | 1 | H | H | A-13 | H | H | H | m-D6 | H | H | H |
| 39823 | 1 | H | H | A-14 | H | H | H | m-D6 | H | H | H |
| 39824 | 1 | H | H | A-15 | H | H | H | m-D6 | H | H | H |
| 39825 | 1 | H | H | A-16 | H | H | H | m-D6 | H | H | H |
| 39826 | 1 | H | H | A-17 | H | H | H | m-D6 | H | H | H |
| 39827 | 1 | H | H | A-18 | H | H | H | m-D6 | H | H | H |
| 39828 | 1 | H | H | A-19 | H | H | H | m-D6 | H | H | H |
| 39829 | 1 | H | H | A-20 | H | H | H | m-D6 | H | H | H |
| 39830 | 1 | H | H | A-21 | H | H | H | m-D6 | H | H | H |
| 39831 | 1 | H | H | A-22 | H | H | H | m-D6 | H | H | H |
| 39832 | 1 | H | H | A-23 | H | H | H | m-D6 | H | H | H |
| 39833 | 1 | H | H | A-24 | H | H | H | m-D6 | H | H | H |
| 39834 | 1 | H | H | A-25 | H | H | H | m-D6 | H | H | H |
| 39835 | 1 | H | H | A-26 | H | H | H | m-D6 | H | H | H |
| 39836 | 1 | H | H | A-27 | H | H | H | m-D6 | H | H | H |
| 39837 | 1 | H | H | A-28 | H | H | H | m-D6 | H | H | H |
| 39838 | 1 | H | H | A-29 | H | H | H | m-D6 | H | H | H |
| 39839 | 1 | H | H | A-30 | H | H | H | m-D6 | H | H | H |
| 39840 | 1 | H | H | A-31 | H | H | H | m-D6 | H | H | H |
| 39841 | 1 | H | H | A-32 | H | H | H | m-D6 | H | H | H |
| 39842 | 1 | H | H | A-33 | H | H | H | m-D6 | H | H | H |
| 39843 | 1 | H | H | A-34 | H | H | H | m-D6 | H | H | H |
| 39844 | 1 | H | H | A-35 | H | H | H | m-D6 | H | H | H |
| 39845 | 1 | H | H | A-36 | H | H | H | m-D6 | H | H | H |
| 39846 | 1 | H | H | A-37 | H | H | H | m-D6 | H | H | H |
| 39847 | 1 | H | H | A-38 | H | H | H | m-D6 | H | H | H |
| 39848 | 1 | H | H | A-39 | H | H | H | m-D6 | H | H | H |
| 39849 | 1 | H | H | A-40 | H | H | H | m-D6 | H | H | H |
| 39850 | 1 | H | H | A-41 | H | H | H | m-D6 | H | H | H |
| 39851 | 1 | H | H | A-42 | H | H | H | m-D6 | H | H | H |
| 39852 | 1 | H | H | A-43 | H | H | H | m-D6 | H | H | H |
| 39853 | 1 | H | H | A-44 | H | H | H | m-D6 | H | H | H |
| 39854 | 1 | H | H | A-45 | H | H | H | m-D6 | H | H | H |
| 39855 | 1 | H | H | A-46 | H | H | H | m-D6 | H | H | H |
| 39856 | 1 | H | H | A-47 | H | H | H | m-D6 | H | H | H |
| 39857 | 1 | H | H | A-1 | H | H | H | m-D7 | H | H | H |
| 39858 | 1 | H | H | A-2 | H | H | H | m-D7 | H | H | H |
| 39859 | 1 | H | H | A-3 | H | H | H | m-D7 | H | H | H |
| 39860 | 1 | H | H | A-4 | H | H | H | m-D7 | H | H | H |
| 39861 | 1 | H | H | A-5 | H | H | H | m-D7 | H | H | H |
| 39862 | 1 | H | H | A-6 | H | H | H | m-D7 | H | H | H |
| 39863 | 1 | H | H | A-7 | H | H | H | m-D7 | H | H | H |
| 39864 | 1 | H | H | A-8 | H | H | H | m-D7 | H | H | H |
| 39865 | 1 | H | H | A-9 | H | H | H | m-D7 | H | H | H |
| 39866 | 1 | H | H | A-10 | H | H | H | m-D7 | H | H | H |
| 39867 | 1 | H | H | A-11 | H | H | H | m-D7 | H | H | H |
| 39868 | 1 | H | H | A-12 | H | H | H | m-D7 | H | H | H |
| 39869 | 1 | H | H | A-13 | H | H | H | m-D7 | H | H | H |
| 39870 | 1 | H | H | A-14 | H | H | H | m-D7 | H | H | H |
| 39871 | 1 | H | H | A-15 | H | H | H | m-D7 | H | H | H |
| 39872 | 1 | H | H | A-16 | H | H | H | m-D7 | H | H | H |
| 39873 | 1 | H | H | A-17 | H | H | H | m-D7 | H | H | H |
| 39874 | 1 | H | H | A-18 | H | H | H | m-D7 | H | H | H |
| 39875 | 1 | H | H | A-19 | H | H | H | m-D7 | H | H | H |
| 39876 | 1 | H | H | A-20 | H | H | H | m-D7 | H | H | H |
| 39877 | 1 | H | H | A-21 | H | H | H | m-D7 | H | H | H |
| 39878 | 1 | H | H | A-22 | H | H | H | m-D7 | H | H | H |
| 39879 | 1 | H | H | A-23 | H | H | H | m-D7 | H | H | H |
| 39880 | 1 | H | H | A-24 | H | H | H | m-D7 | H | H | H |
| 39881 | 1 | H | H | A-25 | H | H | H | m-D7 | H | H | H |
| 39882 | 1 | H | H | A-26 | H | H | H | m-D7 | H | H | H |
| 39883 | 1 | H | H | A-27 | H | H | H | m-D7 | H | H | H |
| 39884 | 1 | H | H | A-28 | H | H | H | m-D7 | H | H | H |
| 39885 | 1 | H | H | A-29 | H | H | H | m-D7 | H | H | H |
| 39886 | 1 | H | H | A-30 | H | H | H | m-D7 | H | H | H |
| 39887 | 1 | H | H | A-31 | H | H | H | m-D7 | H | H | H |
| 39888 | 1 | H | H | A-32 | H | H | H | m-D7 | H | H | H |
| 39889 | 1 | H | H | A-33 | H | H | H | m-D7 | H | H | H |
| 39890 | 1 | H | H | A-34 | H | H | H | m-D7 | H | H | H |
| 39891 | 1 | H | H | A-35 | H | H | H | m-D7 | H | H | H |
| 39892 | 1 | H | H | A-36 | H | H | H | m-D7 | H | H | H |
| 39893 | 1 | H | H | A-37 | H | H | H | m-D7 | H | H | H |
| 39894 | 1 | H | H | A-38 | H | H | H | m-D7 | H | H | H |
| 39895 | 1 | H | H | A-39 | H | H | H | m-D7 | H | H | H |
| 39896 | 1 | H | H | A-40 | H | H | H | m-D7 | H | H | H |
| 39897 | 1 | H | H | A-41 | H | H | H | m-D7 | H | H | H |
| 39898 | 1 | H | H | A-42 | H | H | H | m-D7 | H | H | H |
| 39899 | 1 | H | H | A-43 | H | H | H | m-D7 | H | H | H |
| 39900 | 1 | H | H | A-44 | H | H | H | m-D7 | H | H | H |
| 39901 | 1 | H | H | A-45 | H | H | H | m-D7 | H | H | H |
| 39902 | 1 | H | H | A-46 | H | H | H | m-D7 | H | H | H |
| 39903 | 1 | H | H | A-47 | H | H | H | m-D7 | H | H | H |
| 39904 | 1 | H | H | A-1 | H | H | H | m-D8 | H | H | H |
| 39905 | 1 | H | H | A-2 | H | H | H | m-D8 | H | H | H |
| 39906 | 1 | H | H | A-3 | H | H | H | m-D8 | H | H | H |
| 39907 | 1 | H | H | A-4 | H | H | H | m-D8 | H | H | H |
| 39908 | 1 | H | H | A-5 | H | H | H | m-D8 | H | H | H |
| 39909 | 1 | H | H | A-6 | H | H | H | m-D8 | H | H | H |
| 39910 | 1 | H | H | A-7 | H | H | H | m-D8 | H | H | H |
| 39911 | 1 | H | H | A-8 | H | H | H | m-D8 | H | H | H |
| 39912 | 1 | H | H | A-9 | H | H | H | m-D8 | H | H | H |
| 39913 | 1 | H | H | A-10 | H | H | H | m-D8 | H | H | H |
| 39914 | 1 | H | H | A-11 | H | H | H | m-D8 | H | H | H |
| 39915 | 1 | H | H | A-12 | H | H | H | m-D8 | H | H | H |
| 39916 | 1 | H | H | A-13 | H | H | H | m-D8 | H | H | H |
| 39917 | 1 | H | H | A-14 | H | H | H | m-D8 | H | H | H |
| 39918 | 1 | H | H | A-15 | H | H | H | m-D8 | H | H | H |
| 39919 | 1 | H | H | A-16 | H | H | H | m-D8 | H | H | H |
| 39920 | 1 | H | H | A-17 | H | H | H | m-D8 | H | H | H |
| 39921 | 1 | H | H | A-18 | H | H | H | m-D8 | H | H | H |
| 39922 | 1 | H | H | A-19 | H | H | H | m-D8 | H | H | H |
| 39923 | 1 | H | H | A-20 | H | H | H | m-D8 | H | H | H |
| 39924 | 1 | H | H | A-21 | H | H | H | m-D8 | H | H | H |
| 39925 | 1 | H | H | A-22 | H | H | H | m-D8 | H | H | H |
| 39926 | 1 | H | H | A-23 | H | H | H | m-D8 | H | H | H |
| 39927 | 1 | H | H | A-24 | H | H | H | m-D8 | H | H | H |
| 39928 | 1 | H | H | A-25 | H | H | H | m-D8 | H | H | H |
| 39929 | 1 | H | H | A-26 | H | H | H | m-D8 | H | H | H |
| 39930 | 1 | H | H | A-27 | H | H | H | m-D8 | H | H | H |
| 39931 | 1 | H | H | A-28 | H | H | H | m-D8 | H | H | H |
| 39932 | 1 | H | H | A-29 | H | H | H | m-D8 | H | H | H |
| 39933 | 1 | H | H | A-30 | H | H | H | m-D8 | H | H | H |
| 39934 | 1 | H | H | A-31 | H | H | H | m-D8 | H | H | H |
| 39935 | 1 | H | H | A-32 | H | H | H | m-D8 | H | H | H |
| 39936 | 1 | H | H | A-33 | H | H | H | m-D8 | H | H | H |
| 39937 | 1 | H | H | A-34 | H | H | H | m-D8 | H | H | H |
| 39938 | 1 | H | H | A-35 | H | H | H | m-D8 | H | H | H |
| 39939 | 1 | H | H | A-36 | H | H | H | m-D8 | H | H | H |
| 39940 | 1 | H | H | A-37 | H | H | H | m-D8 | H | H | H |
| 39941 | 1 | H | H | A-38 | H | H | H | m-D8 | H | H | H |
| 39942 | 1 | H | H | A-39 | H | H | H | m-D8 | H | H | H |
| 39943 | 1 | H | H | A-40 | H | H | H | m-D8 | H | H | H |
| 39944 | 1 | H | H | A-41 | H | H | H | m-D8 | H | H | H |
| 39945 | 1 | H | H | A-42 | H | H | H | m-D8 | H | H | H |
| 39946 | 1 | H | H | A-43 | H | H | H | m-D8 | H | H | H |
| 39947 | 1 | H | H | A-44 | H | H | H | m-D8 | H | H | H |
| 39948 | 1 | H | H | A-45 | H | H | H | m-D8 | H | H | H |
| 39949 | 1 | H | H | A-46 | H | H | H | m-D8 | H | H | H |
| 39950 | 1 | H | H | A-47 | H | H | H | m-D8 | H | H | H |
| 39951 | 1 | H | H | A-1 | H | H | H | m-D9 | H | H | H |
| 39952 | 1 | H | H | A-2 | H | H | H | m-D9 | H | H | H |
| 39953 | 1 | H | H | A-3 | H | H | H | m-D9 | H | H | H |
| 39954 | 1 | H | H | A-4 | H | H | H | m-D9 | H | H | H |
| 39955 | 1 | H | H | A-5 | H | H | H | m-D9 | H | H | H |
| 39956 | 1 | H | H | A-6 | H | H | H | m-D9 | H | H | H |
| 39957 | 1 | H | H | A-7 | H | H | H | m-D9 | H | H | H |
| 39958 | 1 | H | H | A-8 | H | H | H | m-D9 | H | H | H |
| 39959 | 1 | H | H | A-9 | H | H | H | m-D9 | H | H | H |
| 39960 | 1 | H | H | A-10 | H | H | H | m-D9 | H | H | H |
| 39961 | 1 | H | H | A-11 | H | H | H | m-D9 | H | H | H |
| 39962 | 1 | H | H | A-12 | H | H | H | m-D9 | H | H | H |
| 39963 | 1 | H | H | A-13 | H | H | H | m-D9 | H | H | H |
| 39964 | 1 | H | H | A-14 | H | H | H | m-D9 | H | H | H |
| 39965 | 1 | H | H | A-15 | H | H | H | m-D9 | H | H | H |
| 39966 | 1 | H | H | A-16 | H | H | H | m-D9 | H | H | H |
| 39967 | 1 | H | H | A-17 | H | H | H | m-D9 | H | H | H |
| 39968 | 1 | H | H | A-18 | H | H | H | m-D9 | H | H | H |
| 39969 | 1 | H | H | A-19 | H | H | H | m-D9 | H | H | H |
| 39970 | 1 | H | H | A-20 | H | H | H | m-D9 | H | H | H |
| 39971 | 1 | H | H | A-21 | H | H | H | m-D9 | H | H | H |
| 39972 | 1 | H | H | A-22 | H | H | H | m-D9 | H | H | H |
| 39973 | 1 | H | H | A-23 | H | H | H | m-D9 | H | H | H |
| 39974 | 1 | H | H | A-24 | H | H | H | m-D9 | H | H | H |
| 39975 | 1 | H | H | A-25 | H | H | H | m-D9 | H | H | H |
| 39976 | 1 | H | H | A-26 | H | H | H | m-D9 | H | H | H |
| 39977 | 1 | H | H | A-27 | H | H | H | m-D9 | H | H | H |
| 39978 | 1 | H | H | A-28 | H | H | H | m-D9 | H | H | H |
| 39979 | 1 | H | H | A-29 | H | H | H | m-D9 | H | H | H |
| 39980 | 1 | H | H | A-30 | H | H | H | m-D9 | H | H | H |
| 39981 | 1 | H | H | A-31 | H | H | H | m-D9 | H | H | H |
| 39982 | 1 | H | H | A-32 | H | H | H | m-D9 | H | H | H |
| 39983 | 1 | H | H | A-33 | H | H | H | m-D9 | H | H | H |
| 39984 | 1 | H | H | A-34 | H | H | H | m-D9 | H | H | H |
| 39985 | 1 | H | H | A-35 | H | H | H | m-D9 | H | H | H |
| 39986 | 1 | H | H | A-36 | H | H | H | m-D9 | H | H | H |
| 39987 | 1 | H | H | A-37 | H | H | H | m-D9 | H | H | H |
| 39988 | 1 | H | H | A-38 | H | H | H | m-D9 | H | H | H |
| 39989 | 1 | H | H | A-39 | H | H | H | m-D9 | H | H | H |
| 39990 | 1 | H | H | A-40 | H | H | H | m-D9 | H | H | H |
| 39991 | 1 | H | H | A-41 | H | H | H | m-D9 | H | H | H |
| 39992 | 1 | H | H | A-42 | H | H | H | m-D9 | H | H | H |
| 39993 | 1 | H | H | A-43 | H | H | H | m-D9 | H | H | H |
| 39994 | 1 | H | H | A-44 | H | H | H | m-D9 | H | H | H |
| 39995 | 1 | H | H | A-45 | H | H | H | m-D9 | H | H | H |
| 39996 | 1 | H | H | A-46 | H | H | H | m-D9 | H | H | H |
| 39997 | 1 | H | H | A-47 | H | H | H | m-D9 | H | H | H |
| 39998 | 1 | H | H | A-1 | H | H | H | m-D10 | H | H | H |
| 39999 | 1 | H | H | A-2 | H | H | H | m-D10 | H | H | H |
| 40000 | 1 | H | H | A-3 | H | H | H | m-D10 | H | H | H |
| 40001 | 1 | H | H | A-4 | H | H | H | m-D10 | H | H | H |
| 40002 | 1 | H | H | A-5 | H | H | H | m-D10 | H | H | H |
| 40003 | 1 | H | H | A-6 | H | H | H | m-D10 | H | H | H |
| 40004 | 1 | H | H | A-7 | H | H | H | m-D10 | H | H | H |
| 40005 | 1 | H | H | A-8 | H | H | H | m-D10 | H | H | H |
| 40006 | 1 | H | H | A-9 | H | H | H | m-D10 | H | H | H |
| 40007 | 1 | H | H | A-10 | H | H | H | m-D10 | H | H | H |
| 40008 | 1 | H | H | A-11 | H | H | H | m-D10 | H | H | H |
| 40009 | 1 | H | H | A-12 | H | H | H | m-D10 | H | H | H |
| 40010 | 1 | H | H | A-13 | H | H | H | m-D10 | H | H | H |
| 40011 | 1 | H | H | A-14 | H | H | H | m-D10 | H | H | H |
| 40012 | 1 | H | H | A-15 | H | H | H | m-D10 | H | H | H |
| 40013 | 1 | H | H | A-16 | H | H | H | m-D10 | H | H | H |
| 40014 | 1 | H | H | A-17 | H | H | H | m-D10 | H | H | H |
| 40015 | 1 | H | H | A-18 | H | H | H | m-D10 | H | H | H |
| 40016 | 1 | H | H | A-19 | H | H | H | m-D10 | H | H | H |
| 40017 | 1 | H | H | A-20 | H | H | H | m-D10 | H | H | H |
| 40018 | 1 | H | H | A-21 | H | H | H | m-D10 | H | H | H |
| 40019 | 1 | H | H | A-22 | H | H | H | m-D10 | H | H | H |
| 40020 | 1 | H | H | A-23 | H | H | H | m-D10 | H | H | H |
| 40021 | 1 | H | H | A-24 | H | H | H | m-D10 | H | H | H |
| 40022 | 1 | H | H | A-25 | H | H | H | m-D10 | H | H | H |
| 40023 | 1 | H | H | A-26 | H | H | H | m-D10 | H | H | H |
| 40024 | 1 | H | H | A-27 | H | H | H | m-D10 | H | H | H |
| 40025 | 1 | H | H | A-28 | H | H | H | m-D10 | H | H | H |
| 40026 | 1 | H | H | A-29 | H | H | H | m-D10 | H | H | H |
| 40027 | 1 | H | H | A-30 | H | H | H | m-D10 | H | H | H |
| 40028 | 1 | H | H | A-31 | H | H | H | m-D10 | H | H | H |
| 40029 | 1 | H | H | A-32 | H | H | H | m-D10 | H | H | H |
| 40030 | 1 | H | H | A-33 | H | H | H | m-D10 | H | H | H |
| 40031 | 1 | H | H | A-34 | H | H | H | m-D10 | H | H | H |
| 40032 | 1 | H | H | A-35 | H | H | H | m-D10 | H | H | H |
| 40033 | 1 | H | H | A-36 | H | H | H | m-D10 | H | H | H |
| 40034 | 1 | H | H | A-37 | H | H | H | m-D10 | H | H | H |
| 40035 | 1 | H | H | A-38 | H | H | H | m-D10 | H | H | H |
| 40036 | 1 | H | H | A-39 | H | H | H | m-D10 | H | H | H |
| 40037 | 1 | H | H | A-40 | H | H | H | m-D10 | H | H | H |
| 40038 | 1 | H | H | A-41 | H | H | H | m-D10 | H | H | H |
| 40039 | 1 | H | H | A-42 | H | H | H | m-D10 | H | H | H |
| 40040 | 1 | H | H | A-43 | H | H | H | m-D10 | H | H | H |
| 40041 | 1 | H | H | A-44 | H | H | H | m-D10 | H | H | H |
| 40042 | 1 | H | H | A-45 | H | H | H | m-D10 | H | H | H |
| 40043 | 1 | H | H | A-46 | H | H | H | m-D10 | H | H | H |
| 40044 | 1 | H | H | A-47 | H | H | H | m-D10 | H | H | H |
| 40045 | 1 | H | H | A-1 | H | H | H | m-D11 | H | H | H |
| 40046 | 1 | H | H | A-2 | H | H | H | m-D11 | H | H | H |
| 40047 | 1 | H | H | A-3 | H | H | H | m-D11 | H | H | H |
| 40048 | 1 | H | H | A-4 | H | H | H | m-D11 | H | H | H |
| 40049 | 1 | H | H | A-5 | H | H | H | m-D11 | H | H | H |
| 40050 | 1 | H | H | A-6 | H | H | H | m-D11 | H | H | H |
| 40051 | 1 | H | H | A-7 | H | H | H | m-D11 | H | H | H |
| 40052 | 1 | H | H | A-8 | H | H | H | m-D11 | H | H | H |
| 40053 | 1 | H | H | A-9 | H | H | H | m-D11 | H | H | H |
| 40054 | 1 | H | H | A-10 | H | H | H | m-D11 | H | H | H |
| 40055 | 1 | H | H | A-11 | H | H | H | m-D11 | H | H | H |
| 40056 | 1 | H | H | A-12 | H | H | H | m-D11 | H | H | H |
| 40057 | 1 | H | H | A-13 | H | H | H | m-D11 | H | H | H |
| 40058 | 1 | H | H | A-14 | H | H | H | m-D11 | H | H | H |
| 40059 | 1 | H | H | A-15 | H | H | H | m-D11 | H | H | H |
| 40060 | 1 | H | H | A-16 | H | H | H | m-D11 | H | H | H |
| 40061 | 1 | H | H | A-17 | H | H | H | m-D11 | H | H | H |
| 40062 | 1 | H | H | A-18 | H | H | H | m-D11 | H | H | H |
| 40063 | 1 | H | H | A-19 | H | H | H | m-D11 | H | H | H |
| 40064 | 1 | H | H | A-20 | H | H | H | m-D11 | H | H | H |
| 40065 | 1 | H | H | A-21 | H | H | H | m-D11 | H | H | H |
| 40066 | 1 | H | H | A-22 | H | H | H | m-D11 | H | H | H |
| 40067 | 1 | H | H | A-23 | H | H | H | m-D11 | H | H | H |
| 40068 | 1 | H | H | A-24 | H | H | H | m-D11 | H | H | H |
| 40069 | 1 | H | H | A-25 | H | H | H | m-D11 | H | H | H |
| 40070 | 1 | H | H | A-26 | H | H | H | m-D11 | H | H | H |
| 40071 | 1 | H | H | A-27 | H | H | H | m-D11 | H | H | H |
| 40072 | 1 | H | H | A-28 | H | H | H | m-D11 | H | H | H |
| 40073 | 1 | H | H | A-29 | H | H | H | m-D11 | H | H | H |
| 40074 | 1 | H | H | A-30 | H | H | H | m-D11 | H | H | H |
| 40075 | 1 | H | H | A-31 | H | H | H | m-D11 | H | H | H |
| 40076 | 1 | H | H | A-32 | H | H | H | m-D11 | H | H | H |
| 40077 | 1 | H | H | A-33 | H | H | H | m-D11 | H | H | H |
| 40078 | 1 | H | H | A-34 | H | H | H | m-D11 | H | H | H |
| 40079 | 1 | H | H | A-35 | H | H | H | m-D11 | H | H | H |
| 40080 | 1 | H | H | A-36 | H | H | H | m-D11 | H | H | H |
| 40081 | 1 | H | H | A-37 | H | H | H | m-D11 | H | H | H |
| 40082 | 1 | H | H | A-38 | H | H | H | m-D11 | H | H | H |
| 40083 | 1 | H | H | A-39 | H | H | H | m-D11 | H | H | H |
| 40084 | 1 | H | H | A-40 | H | H | H | m-D11 | H | H | H |
| 40085 | 1 | H | H | A-41 | H | H | H | m-D11 | H | H | H |
| 40086 | 1 | H | H | A-42 | H | H | H | m-D11 | H | H | H |
| 40087 | 1 | H | H | A-43 | H | H | H | m-D11 | H | H | H |
| 40088 | 1 | H | H | A-44 | H | H | H | m-D11 | H | H | H |
| 40089 | 1 | H | H | A-45 | H | H | H | m-D11 | H | H | H |
| 40090 | 1 | H | H | A-46 | H | H | H | m-D11 | H | H | H |
| 40091 | 1 | H | H | A-47 | H | H | H | m-D11 | H | H | H |
| 40092 | 1 | H | H | A-1 | H | H | H | m-D12 | H | H | H |
| 40093 | 1 | H | H | A-2 | H | H | H | m-D12 | H | H | H |
| 40094 | 1 | H | H | A-3 | H | H | H | m-D12 | H | H | H |
| 40095 | 1 | H | H | A-4 | H | H | H | m-D12 | H | H | H |
| 40096 | 1 | H | H | A-5 | H | H | H | m-D12 | H | H | H |
| 40097 | 1 | H | H | A-6 | H | H | H | m-D12 | H | H | H |
| 40098 | 1 | H | H | A-7 | H | H | H | m-D12 | H | H | H |
| 40099 | 1 | H | H | A-8 | H | H | H | m-D12 | H | H | H |
| 40100 | 1 | H | H | A-9 | H | H | H | m-D12 | H | H | H |
| 40101 | 1 | H | H | A-10 | H | H | H | m-D12 | H | H | H |
| 40102 | 1 | H | H | A-11 | H | H | H | m-D12 | H | H | H |
| 40103 | 1 | H | H | A-12 | H | H | H | m-D12 | H | H | H |
| 40104 | 1 | H | H | A-13 | H | H | H | m-D12 | H | H | H |
| 40105 | 1 | H | H | A-14 | H | H | H | m-D12 | H | H | H |
| 40106 | 1 | H | H | A-15 | H | H | H | m-D12 | H | H | H |
| 40107 | 1 | H | H | A-16 | H | H | H | m-D12 | H | H | H |
| 40108 | 1 | H | H | A-17 | H | H | H | m-D12 | H | H | H |
| 40109 | 1 | H | H | A-18 | H | H | H | m-D12 | H | H | H |
| 40110 | 1 | H | H | A-19 | H | H | H | m-D12 | H | H | H |
| 40111 | 1 | H | H | A-20 | H | H | H | m-D12 | H | H | H |
| 40112 | 1 | H | H | A-21 | H | H | H | m-D12 | H | H | H |
| 40113 | 1 | H | H | A-22 | H | H | H | m-D12 | H | H | H |
| 40114 | 1 | H | H | A-23 | H | H | H | m-D12 | H | H | H |
| 40115 | 1 | H | H | A-24 | H | H | H | m-D12 | H | H | H |
| 40116 | 1 | H | H | A-25 | H | H | H | m-D12 | H | H | H |
| 40117 | 1 | H | H | A-26 | H | H | H | m-D12 | H | H | H |
| 40118 | 1 | H | H | A-27 | H | H | H | m-D12 | H | H | H |
| 40119 | 1 | H | H | A-28 | H | H | H | m-D12 | H | H | H |
| 40120 | 1 | H | H | A-29 | H | H | H | m-D12 | H | H | H |
| 40121 | 1 | H | H | A-30 | H | H | H | m-D12 | H | H | H |
| 40122 | 1 | H | H | A-31 | H | H | H | m-D12 | H | H | H |
| 40123 | 1 | H | H | A-32 | H | H | H | m-D12 | H | H | H |
| 40124 | 1 | H | H | A-33 | H | H | H | m-D12 | H | H | H |
| 40125 | 1 | H | H | A-34 | H | H | H | m-D12 | H | H | H |
| 40126 | 1 | H | H | A-35 | H | H | H | m-D12 | H | H | H |
| 40127 | 1 | H | H | A-36 | H | H | H | m-D12 | H | H | H |
| 40128 | 1 | H | H | A-37 | H | H | H | m-D12 | H | H | H |
| 40129 | 1 | H | H | A-38 | H | H | H | m-D12 | H | H | H |
| 40130 | 1 | H | H | A-39 | H | H | H | m-D12 | H | H | H |
| 40131 | 1 | H | H | A-40 | H | H | H | m-D12 | H | H | H |
| 40132 | 1 | H | H | A-41 | H | H | H | m-D12 | H | H | H |
| 40133 | 1 | H | H | A-42 | H | H | H | m-D12 | H | H | H |
| 40134 | 1 | H | H | A-43 | H | H | H | m-D12 | H | H | H |
| 40135 | 1 | H | H | A-44 | H | H | H | m-D12 | H | H | H |
| 40136 | 1 | H | H | A-45 | H | H | H | m-D12 | H | H | H |
| 40137 | 1 | H | H | A-46 | H | H | H | m-D12 | H | H | H |
| 40138 | 1 | H | H | A-47 | H | H | H | m-D12 | H | H | H |
| 40139 | 1 | H | H | A-1 | H | H | H | m-D13 | H | H | H |
| 40140 | 1 | H | H | A-2 | H | H | H | m-D13 | H | H | H |
| 40141 | 1 | H | H | A-3 | H | H | H | m-D13 | H | H | H |
| 40142 | 1 | H | H | A-4 | H | H | H | m-D13 | H | H | H |
| 40143 | 1 | H | H | A-5 | H | H | H | m-D13 | H | H | H |
| 40144 | 1 | H | H | A-6 | H | H | H | m-D13 | H | H | H |
| 40145 | 1 | H | H | A-7 | H | H | H | m-D13 | H | H | H |
| 40146 | 1 | H | H | A-8 | H | H | H | m-D13 | H | H | H |
| 40147 | 1 | H | H | A-9 | H | H | H | m-D13 | H | H | H |
| 40148 | 1 | H | H | A-10 | H | H | H | m-D13 | H | H | H |
| 40149 | 1 | H | H | A-11 | H | H | H | m-D13 | H | H | H |
| 40150 | 1 | H | H | A-12 | H | H | H | m-D13 | H | H | H |
| 40151 | 1 | H | H | A-13 | H | H | H | m-D13 | H | H | H |
| 40152 | 1 | H | H | A-14 | H | H | H | m-D13 | H | H | H |
| 40153 | 1 | H | H | A-15 | H | H | H | m-D13 | H | H | H |
| 40154 | 1 | H | H | A-16 | H | H | H | m-D13 | H | H | H |
| 40155 | 1 | H | H | A-17 | H | H | H | m-D13 | H | H | H |
| 40156 | 1 | H | H | A-18 | H | H | H | m-D13 | H | H | H |
| 40157 | 1 | H | H | A-19 | H | H | H | m-D13 | H | H | H |
| 40158 | 1 | H | H | A-20 | H | H | H | m-D13 | H | H | H |
| 40159 | 1 | H | H | A-21 | H | H | H | m-D13 | H | H | H |
| 40160 | 1 | H | H | A-22 | H | H | H | m-D13 | H | H | H |
| 40161 | 1 | H | H | A-23 | H | H | H | m-D13 | H | H | H |
| 40162 | 1 | H | H | A-24 | H | H | H | m-D13 | H | H | H |
| 40163 | 1 | H | H | A-25 | H | H | H | m-D13 | H | H | H |
| 40164 | 1 | H | H | A-26 | H | H | H | m-D13 | H | H | H |
| 40165 | 1 | H | H | A-27 | H | H | H | m-D13 | H | H | H |
| 40166 | 1 | H | H | A-28 | H | H | H | m-D13 | H | H | H |
| 40167 | 1 | H | H | A-29 | H | H | H | m-D13 | H | H | H |
| 40168 | 1 | H | H | A-30 | H | H | H | m-D13 | H | H | H |
| 40169 | 1 | H | H | A-31 | H | H | H | m-D13 | H | H | H |
| 40170 | 1 | H | H | A-32 | H | H | H | m-D13 | H | H | H |
| 40171 | 1 | H | H | A-33 | H | H | H | m-D13 | H | H | H |
| 40172 | 1 | H | H | A-34 | H | H | H | m-D13 | H | H | H |
| 40173 | 1 | H | H | A-35 | H | H | H | m-D13 | H | H | H |
| 40174 | 1 | H | H | A-36 | H | H | H | m-D13 | H | H | H |
| 40175 | 1 | H | H | A-37 | H | H | H | m-D13 | H | H | H |
| 40176 | 1 | H | H | A-38 | H | H | H | m-D13 | H | H | H |
| 40177 | 1 | H | H | A-39 | H | H | H | m-D13 | H | H | H |
| 40178 | 1 | H | H | A-40 | H | H | H | m-D13 | H | H | H |
| 40179 | 1 | H | H | A-41 | H | H | H | m-D13 | H | H | H |
| 40180 | 1 | H | H | A-42 | H | H | H | m-D13 | H | H | H |
| 40181 | 1 | H | H | A-43 | H | H | H | m-D13 | H | H | H |
| 40182 | 1 | H | H | A-44 | H | H | H | m-D13 | H | H | H |
| 40183 | 1 | H | H | A-45 | H | H | H | m-D13 | H | H | H |
| 40184 | 1 | H | H | A-46 | H | H | H | m-D13 | H | H | H |
| 40185 | 1 | H | H | A-47 | H | H | H | m-D13 | H | H | H |
| 40186 | 1 | H | H | A-1 | H | H | H | m-D14 | H | H | H |
| 40187 | 1 | H | H | A-2 | H | H | H | m-D14 | H | H | H |
| 40188 | 1 | H | H | A-3 | H | H | H | m-D14 | H | H | H |
| 40189 | 1 | H | H | A-4 | H | H | H | m-D14 | H | H | H |
| 40190 | 1 | H | H | A-5 | H | H | H | m-D14 | H | H | H |
| 40191 | 1 | H | H | A-6 | H | H | H | m-D14 | H | H | H |
| 40192 | 1 | H | H | A-7 | H | H | H | m-D14 | H | H | H |
| 40193 | 1 | H | H | A-8 | H | H | H | m-D14 | H | H | H |
| 40194 | 1 | H | H | A-9 | H | H | H | m-D14 | H | H | H |
| 40195 | 1 | H | H | A-10 | H | H | H | m-D14 | H | H | H |
| 40196 | 1 | H | H | A-11 | H | H | H | m-D14 | H | H | H |
| 40197 | 1 | H | H | A-12 | H | H | H | m-D14 | H | H | H |
| 40198 | 1 | H | H | A-13 | H | H | H | m-D14 | H | H | H |
| 40199 | 1 | H | H | A-14 | H | H | H | m-D14 | H | H | H |
| 40200 | 1 | H | H | A-15 | H | H | H | m-D14 | H | H | H |
| 40201 | 1 | H | H | A-16 | H | H | H | m-D14 | H | H | H |
| 40202 | 1 | H | H | A-17 | H | H | H | m-D14 | H | H | H |
| 40203 | 1 | H | H | A-18 | H | H | H | m-D14 | H | H | H |
| 40204 | 1 | H | H | A-19 | H | H | H | m-D14 | H | H | H |
| 40205 | 1 | H | H | A-20 | H | H | H | m-D14 | H | H | H |
| 40206 | 1 | H | H | A-21 | H | H | H | m-D14 | H | H | H |
| 40207 | 1 | H | H | A-22 | H | H | H | m-D14 | H | H | H |
| 40208 | 1 | H | H | A-23 | H | H | H | m-D14 | H | H | H |
| 40209 | 1 | H | H | A-24 | H | H | H | m-D14 | H | H | H |
| 40210 | 1 | H | H | A-25 | H | H | H | m-D14 | H | H | H |
| 40211 | 1 | H | H | A-26 | H | H | H | m-D14 | H | H | H |
| 40212 | 1 | H | H | A-27 | H | H | H | m-D14 | H | H | H |
| 40213 | 1 | H | H | A-28 | H | H | H | m-D14 | H | H | H |
| 40214 | 1 | H | H | A-29 | H | H | H | m-D14 | H | H | H |
| 40215 | 1 | H | H | A-30 | H | H | H | m-D14 | H | H | H |
| 40216 | 1 | H | H | A-31 | H | H | H | m-D14 | H | H | H |
| 40217 | 1 | H | H | A-32 | H | H | H | m-D14 | H | H | H |
| 40218 | 1 | H | H | A-33 | H | H | H | m-D14 | H | H | H |
| 40219 | 1 | H | H | A-34 | H | H | H | m-D14 | H | H | H |
| 40220 | 1 | H | H | A-35 | H | H | H | m-D14 | H | H | H |
| 40221 | 1 | H | H | A-36 | H | H | H | m-D14 | H | H | H |
| 40222 | 1 | H | H | A-37 | H | H | H | m-D14 | H | H | H |
| 40223 | 1 | H | H | A-38 | H | H | H | m-D14 | H | H | H |
| 40224 | 1 | H | H | A-39 | H | H | H | m-D14 | H | H | H |
| 40225 | 1 | H | H | A-40 | H | H | H | m-D14 | H | H | H |
| 40226 | 1 | H | H | A-41 | H | H | H | m-D14 | H | H | H |
| 40227 | 1 | H | H | A-42 | H | H | H | m-D14 | H | H | H |
| 40228 | 1 | H | H | A-43 | H | H | H | m-D14 | H | H | H |
| 40229 | 1 | H | H | A-44 | H | H | H | m-D14 | H | H | H |
| 40230 | 1 | H | H | A-45 | H | H | H | m-D14 | H | H | H |
| 40231 | 1 | H | H | A-46 | H | H | H | m-D14 | H | H | H |
| 40232 | 1 | H | H | A-47 | H | H | H | m-D14 | H | H | H |
| 40233 | 1 | H | m-D1 | H | H | H | H | A-1 | H | H | H |
| 40234 | 1 | H | m-D1 | H | H | H | H | A-2 | H | H | H |
| 40235 | 1 | H | m-D1 | H | H | H | H | A-3 | H | H | H |
| 40236 | 1 | H | m-D1 | H | H | H | H | A-4 | H | H | H |
| 40237 | 1 | H | m-D1 | H | H | H | H | A-5 | H | H | H |
| 40238 | 1 | H | m-D1 | H | H | H | H | A-6 | H | H | H |
| 40239 | 1 | H | m-D1 | H | H | H | H | A-7 | H | H | H |
| 40240 | 1 | H | m-D1 | H | H | H | H | A-8 | H | H | H |
| 40241 | 1 | H | m-D1 | H | H | H | H | A-9 | H | H | H |
| 40242 | 1 | H | m-D1 | H | H | H | H | A-10 | H | H | H |
| 40243 | 1 | H | m-D1 | H | H | H | H | A-11 | H | H | H |
| 40244 | 1 | H | m-D1 | H | H | H | H | A-12 | H | H | H |
| 40245 | 1 | H | m-D1 | H | H | H | H | A-13 | H | H | H |
| 40246 | 1 | H | m-D1 | H | H | H | H | A-14 | H | H | H |
| 40247 | 1 | H | m-D1 | H | H | H | H | A-15 | H | H | H |
| 40248 | 1 | H | m-D1 | H | H | H | H | A-16 | H | H | H |
| 40249 | 1 | H | m-D1 | H | H | H | H | A-17 | H | H | H |
| 40250 | 1 | H | m-D1 | H | H | H | H | A-18 | H | H | H |
| 40251 | 1 | H | m-D1 | H | H | H | H | A-19 | H | H | H |
| 40252 | 1 | H | m-D1 | H | H | H | H | A-20 | H | H | H |
| 40253 | 1 | H | m-D1 | H | H | H | H | A-21 | H | H | H |
| 40254 | 1 | H | m-D1 | H | H | H | H | A-22 | H | H | H |
| 40255 | 1 | H | m-D1 | H | H | H | H | A-23 | H | H | H |
| 40256 | 1 | H | m-D1 | H | H | H | H | A-24 | H | H | H |
| 40257 | 1 | H | m-D1 | H | H | H | H | A-25 | H | H | H |
| 40258 | 1 | H | m-D1 | H | H | H | H | A-26 | H | H | H |
| 40259 | 1 | H | m-D1 | H | H | H | H | A-27 | H | H | H |
| 40260 | 1 | H | m-D1 | H | H | H | H | A-28 | H | H | H |
| 40261 | 1 | H | m-D1 | H | H | H | H | A-29 | H | H | H |
| 40262 | 1 | H | m-D1 | H | H | H | H | A-30 | H | H | H |
| 40263 | 1 | H | m-D1 | H | H | H | H | A-31 | H | H | H |
| 40264 | 1 | H | m-D1 | H | H | H | H | A-32 | H | H | H |
| 40265 | 1 | H | m-D1 | H | H | H | H | A-33 | H | H | H |
| 40266 | 1 | H | m-D1 | H | H | H | H | A-34 | H | H | H |
| 40267 | 1 | H | m-D1 | H | H | H | H | A-35 | H | H | H |
| 40268 | 1 | H | m-D1 | H | H | H | H | A-36 | H | H | H |
| 40269 | 1 | H | m-D1 | H | H | H | H | A-37 | H | H | H |
| 40270 | 1 | H | m-D1 | H | H | H | H | A-38 | H | H | H |
| 40271 | 1 | H | m-D1 | H | H | H | H | A-39 | H | H | H |
| 40272 | 1 | H | m-D1 | H | H | H | H | A-40 | H | H | H |
| 40273 | 1 | H | m-D1 | H | H | H | H | A-41 | H | H | H |
| 40274 | 1 | H | m-D1 | H | H | H | H | A-42 | H | H | H |
| 40275 | 1 | H | m-D1 | H | H | H | H | A-43 | H | H | H |
| 40276 | 1 | H | m-D1 | H | H | H | H | A-44 | H | H | H |
| 40277 | 1 | H | m-D1 | H | H | H | H | A-45 | H | H | H |
| 40278 | 1 | H | m-D1 | H | H | H | H | A-46 | H | H | H |
| 40279 | 1 | H | m-D1 | H | H | H | H | A-47 | H | H | H |
| 40280 | 1 | H | m-D2 | H | H | H | H | A-1 | H | H | H |
| 40281 | 1 | H | m-D2 | H | H | H | H | A-2 | H | H | H |
| 40282 | 1 | H | m-D2 | H | H | H | H | A-3 | H | H | H |
| 40283 | 1 | H | m-D2 | H | H | H | H | A-4 | H | H | H |
| 40284 | 1 | H | m-D2 | H | H | H | H | A-5 | H | H | H |
| 40285 | 1 | H | m-D2 | H | H | H | H | A-6 | H | H | H |
| 40286 | 1 | H | m-D2 | H | H | H | H | A-7 | H | H | H |
| 40287 | 1 | H | m-D2 | H | H | H | H | A-8 | H | H | H |
| 40288 | 1 | H | m-D2 | H | H | H | H | A-9 | H | H | H |
| 40289 | 1 | H | m-D2 | H | H | H | H | A-10 | H | H | H |
| 40290 | 1 | H | m-D2 | H | H | H | H | A-11 | H | H | H |
| 40291 | 1 | H | m-D2 | H | H | H | H | A-12 | H | H | H |
| 40292 | 1 | H | m-D2 | H | H | H | H | A-13 | H | H | H |
| 40293 | 1 | H | m-D2 | H | H | H | H | A-14 | H | H | H |
| 40294 | 1 | H | m-D2 | H | H | H | H | A-15 | H | H | H |
| 40295 | 1 | H | m-D2 | H | H | H | H | A-16 | H | H | H |
| 40296 | 1 | H | m-D2 | H | H | H | H | A-17 | H | H | H |
| 40297 | 1 | H | m-D2 | H | H | H | H | A-18 | H | H | H |
| 40298 | 1 | H | m-D2 | H | H | H | H | A-19 | H | H | H |
| 40299 | 1 | H | m-D2 | H | H | H | H | A-20 | H | H | H |
| 40300 | 1 | H | m-D2 | H | H | H | H | A-21 | H | H | H |
| 40301 | 1 | H | m-D2 | H | H | H | H | A-22 | H | H | H |
| 40302 | 1 | H | m-D2 | H | H | H | H | A-23 | H | H | H |
| 40303 | 1 | H | m-D2 | H | H | H | H | A-24 | H | H | H |
| 40304 | 1 | H | m-D2 | H | H | H | H | A-25 | H | H | H |
| 40305 | 1 | H | m-D2 | H | H | H | H | A-26 | H | H | H |
| 40306 | 1 | H | m-D2 | H | H | H | H | A-27 | H | H | H |
| 40307 | 1 | H | m-D2 | H | H | H | H | A-28 | H | H | H |
| 40308 | 1 | H | m-D2 | H | H | H | H | A-29 | H | H | H |
| 40309 | 1 | H | m-D2 | H | H | H | H | A-30 | H | H | H |
| 40310 | 1 | H | m-D2 | H | H | H | H | A-31 | H | H | H |
| 40311 | 1 | H | m-D2 | H | H | H | H | A-32 | H | H | H |
| 40312 | 1 | H | m-D2 | H | H | H | H | A-33 | H | H | H |
| 40313 | 1 | H | m-D2 | H | H | H | H | A-34 | H | H | H |
| 40314 | 1 | H | m-D2 | H | H | H | H | A-35 | H | H | H |
| 40315 | 1 | H | m-D2 | H | H | H | H | A-36 | H | H | H |
| 40316 | 1 | H | m-D2 | H | H | H | H | A-37 | H | H | H |
| 40317 | 1 | H | m-D2 | H | H | H | H | A-38 | H | H | H |
| 40318 | 1 | H | m-D2 | H | H | H | H | A-39 | H | H | H |
| 40319 | 1 | H | m-D2 | H | H | H | H | A-40 | H | H | H |
| 40320 | 1 | H | m-D2 | H | H | H | H | A-41 | H | H | H |
| 40321 | 1 | H | m-D2 | H | H | H | H | A-42 | H | H | H |
| 40322 | 1 | H | m-D2 | H | H | H | H | A-43 | H | H | H |
| 40323 | 1 | H | m-D2 | H | H | H | H | A-44 | H | H | H |
| 40324 | 1 | H | m-D2 | H | H | H | H | A-45 | H | H | H |
| 40325 | 1 | H | m-D2 | H | H | H | H | A-46 | H | H | H |
| 40326 | 1 | H | m-D2 | H | H | H | H | A-47 | H | H | H |
| 40327 | 1 | H | m-D3 | H | H | H | H | A-1 | H | H | H |
| 40328 | 1 | H | m-D3 | H | H | H | H | A-2 | H | H | H |
| 40329 | 1 | H | m-D3 | H | H | H | H | A-3 | H | H | H |
| 40330 | 1 | H | m-D3 | H | H | H | H | A-4 | H | H | H |
| 40331 | 1 | H | m-D3 | H | H | H | H | A-5 | H | H | H |
| 40332 | 1 | H | m-D3 | H | H | H | H | A-6 | H | H | H |
| 40333 | 1 | H | m-D3 | H | H | H | H | A-7 | H | H | H |
| 40334 | 1 | H | m-D3 | H | H | H | H | A-8 | H | H | H |
| 40335 | 1 | H | m-D3 | H | H | H | H | A-9 | H | H | H |
| 40336 | 1 | H | m-D3 | H | H | H | H | A-10 | H | H | H |
| 40337 | 1 | H | m-D3 | H | H | H | H | A-11 | H | H | H |
| 40338 | 1 | H | m-D3 | H | H | H | H | A-12 | H | H | H |
| 40339 | 1 | H | m-D3 | H | H | H | H | A-13 | H | H | H |
| 40340 | 1 | H | m-D3 | H | H | H | H | A-14 | H | H | H |
| 40341 | 1 | H | m-D3 | H | H | H | H | A-15 | H | H | H |
| 40342 | 1 | H | m-D3 | H | H | H | H | A-16 | H | H | H |
| 40343 | 1 | H | m-D3 | H | H | H | H | A-17 | H | H | H |
| 40344 | 1 | H | m-D3 | H | H | H | H | A-18 | H | H | H |
| 40345 | 1 | H | m-D3 | H | H | H | H | A-19 | H | H | H |
| 40346 | 1 | H | m-D3 | H | H | H | H | A-20 | H | H | H |
| 40347 | 1 | H | m-D3 | H | H | H | H | A-21 | H | H | H |
| 40348 | 1 | H | m-D3 | H | H | H | H | A-22 | H | H | H |
| 40349 | 1 | H | m-D3 | H | H | H | H | A-23 | H | H | H |
| 40350 | 1 | H | m-D3 | H | H | H | H | A-24 | H | H | H |
| 40351 | 1 | H | m-D3 | H | H | H | H | A-25 | H | H | H |
| 40352 | 1 | H | m-D3 | H | H | H | H | A-26 | H | H | H |
| 40353 | 1 | H | m-D3 | H | H | H | H | A-27 | H | H | H |
| 40354 | 1 | H | m-D3 | H | H | H | H | A-28 | H | H | H |
| 40355 | 1 | H | m-D3 | H | H | H | H | A-29 | H | H | H |
| 40356 | 1 | H | m-D3 | H | H | H | H | A-30 | H | H | H |
| 40357 | 1 | H | m-D3 | H | H | H | H | A-31 | H | H | H |
| 40358 | 1 | H | m-D3 | H | H | H | H | A-32 | H | H | H |
| 40359 | 1 | H | m-D3 | H | H | H | H | A-33 | H | H | H |
| 40360 | 1 | H | m-D3 | H | H | H | H | A-34 | H | H | H |
| 40361 | 1 | H | m-D3 | H | H | H | H | A-35 | H | H | H |
| 40362 | 1 | H | m-D3 | H | H | H | H | A-36 | H | H | H |
| 40363 | 1 | H | m-D3 | H | H | H | H | A-37 | H | H | H |
| 40364 | 1 | H | m-D3 | H | H | H | H | A-38 | H | H | H |
| 40365 | 1 | H | m-D3 | H | H | H | H | A-39 | H | H | H |
| 40366 | 1 | H | m-D3 | H | H | H | H | A-40 | H | H | H |
| 40367 | 1 | H | m-D3 | H | H | H | H | A-41 | H | H | H |
| 40368 | 1 | H | m-D3 | H | H | H | H | A-42 | H | H | H |
| 40369 | 1 | H | m-D3 | H | H | H | H | A-43 | H | H | H |
| 40370 | 1 | H | m-D3 | H | H | H | H | A-44 | H | H | H |
| 40371 | 1 | H | m-D3 | H | H | H | H | A-45 | H | H | H |
| 40372 | 1 | H | m-D3 | H | H | H | H | A-46 | H | H | H |
| 40373 | 1 | H | m-D3 | H | H | H | H | A-47 | H | H | H |
| 40374 | 1 | H | m-D4 | H | H | H | H | A-1 | H | H | H |
| 40375 | 1 | H | m-D4 | H | H | H | H | A-2 | H | H | H |
| 40376 | 1 | H | m-D4 | H | H | H | H | A-3 | H | H | H |
| 40377 | 1 | H | m-D4 | H | H | H | H | A-4 | H | H | H |
| 40378 | 1 | H | m-D4 | H | H | H | H | A-5 | H | H | H |
| 40379 | 1 | H | m-D4 | H | H | H | H | A-6 | H | H | H |
| 40380 | 1 | H | m-D4 | H | H | H | H | A-7 | H | H | H |
| 40381 | 1 | H | m-D4 | H | H | H | H | A-8 | H | H | H |
| 40382 | 1 | H | m-D4 | H | H | H | H | A-9 | H | H | H |
| 40383 | 1 | H | m-D4 | H | H | H | H | A-10 | H | H | H |
| 40384 | 1 | H | m-D4 | H | H | H | H | A-11 | H | H | H |
| 40385 | 1 | H | m-D4 | H | H | H | H | A-12 | H | H | H |
| 40386 | 1 | H | m-D4 | H | H | H | H | A-13 | H | H | H |
| 40387 | 1 | H | m-D4 | H | H | H | H | A-14 | H | H | H |
| 40388 | 1 | H | m-D4 | H | H | H | H | A-15 | H | H | H |
| 40389 | 1 | H | m-D4 | H | H | H | H | A-16 | H | H | H |
| 40390 | 1 | H | m-D4 | H | H | H | H | A-17 | H | H | H |
| 40391 | 1 | H | m-D4 | H | H | H | H | A-18 | H | H | H |
| 40392 | 1 | H | m-D4 | H | H | H | H | A-19 | H | H | H |
| 40393 | 1 | H | m-D4 | H | H | H | H | A-20 | H | H | H |
| 40394 | 1 | H | m-D4 | H | H | H | H | A-21 | H | H | H |
| 40395 | 1 | H | m-D4 | H | H | H | H | A-22 | H | H | H |
| 40396 | 1 | H | m-D4 | H | H | H | H | A-23 | H | H | H |
| 40397 | 1 | H | m-D4 | H | H | H | H | A-24 | H | H | H |
| 40398 | 1 | H | m-D4 | H | H | H | H | A-25 | H | H | H |
| 40399 | 1 | H | m-D4 | H | H | H | H | A-26 | H | H | H |
| 40400 | 1 | H | m-D4 | H | H | H | H | A-27 | H | H | H |
| 40401 | 1 | H | m-D4 | H | H | H | H | A-28 | H | H | H |
| 40402 | 1 | H | m-D4 | H | H | H | H | A-29 | H | H | H |
| 40403 | 1 | H | m-D4 | H | H | H | H | A-30 | H | H | H |
| 40404 | 1 | H | m-D4 | H | H | H | H | A-31 | H | H | H |
| 40405 | 1 | H | m-D4 | H | H | H | H | A-32 | H | H | H |
| 40406 | 1 | H | m-D4 | H | H | H | H | A-33 | H | H | H |
| 40407 | 1 | H | m-D4 | H | H | H | H | A-34 | H | H | H |
| 40408 | 1 | H | m-D4 | H | H | H | H | A-35 | H | H | H |
| 40409 | 1 | H | m-D4 | H | H | H | H | A-36 | H | H | H |
| 40410 | 1 | H | m-D4 | H | H | H | H | A-37 | H | H | H |
| 40411 | 1 | H | m-D4 | H | H | H | H | A-38 | H | H | H |
| 40412 | 1 | H | m-D4 | H | H | H | H | A-39 | H | H | H |
| 40413 | 1 | H | m-D4 | H | H | H | H | A-40 | H | H | H |
| 40414 | 1 | H | m-D4 | H | H | H | H | A-41 | H | H | H |
| 40415 | 1 | H | m-D4 | H | H | H | H | A-42 | H | H | H |
| 40416 | 1 | H | m-D4 | H | H | H | H | A-43 | H | H | H |
| 40417 | 1 | H | m-D4 | H | H | H | H | A-44 | H | H | H |
| 40418 | 1 | H | m-D4 | H | H | H | H | A-45 | H | H | H |
| 40419 | 1 | H | m-D4 | H | H | H | H | A-46 | H | H | H |
| 40420 | 1 | H | m-D4 | H | H | H | H | A-47 | H | H | H |
| 40421 | 1 | H | m-D5 | H | H | H | H | A-1 | H | H | H |
| 40422 | 1 | H | m-D5 | H | H | H | H | A-2 | H | H | H |
| 40423 | 1 | H | m-D5 | H | H | H | H | A-3 | H | H | H |
| 40424 | 1 | H | m-D5 | H | H | H | H | A-4 | H | H | H |
| 40425 | 1 | H | m-D5 | H | H | H | H | A-5 | H | H | H |
| 40426 | 1 | H | m-D5 | H | H | H | H | A-6 | H | H | H |
| 40427 | 1 | H | m-D5 | H | H | H | H | A-7 | H | H | H |
| 40428 | 1 | H | m-D5 | H | H | H | H | A-8 | H | H | H |
| 40429 | 1 | H | m-D5 | H | H | H | H | A-9 | H | H | H |
| 40430 | 1 | H | m-D5 | H | H | H | H | A-10 | H | H | H |
| 40431 | 1 | H | m-D5 | H | H | H | H | A-11 | H | H | H |
| 40432 | 1 | H | m-D5 | H | H | H | H | A-12 | H | H | H |
| 40433 | 1 | H | m-D5 | H | H | H | H | A-13 | H | H | H |
| 40434 | 1 | H | m-D5 | H | H | H | H | A-14 | H | H | H |
| 40435 | 1 | H | m-D5 | H | H | H | H | A-15 | H | H | H |
| 40436 | 1 | H | m-D5 | H | H | H | H | A-16 | H | H | H |
| 40437 | 1 | H | m-D5 | H | H | H | H | A-17 | H | H | H |
| 40438 | 1 | H | m-D5 | H | H | H | H | A-18 | H | H | H |
| 40439 | 1 | H | m-D5 | H | H | H | H | A-19 | H | H | H |
| 40440 | 1 | H | m-D5 | H | H | H | H | A-20 | H | H | H |
| 40441 | 1 | H | m-D5 | H | H | H | H | A-21 | H | H | H |
| 40442 | 1 | H | m-D5 | H | H | H | H | A-22 | H | H | H |
| 40443 | 1 | H | m-D5 | H | H | H | H | A-23 | H | H | H |
| 40444 | 1 | H | m-D5 | H | H | H | H | A-24 | H | H | H |
| 40445 | 1 | H | m-D5 | H | H | H | H | A-25 | H | H | H |
| 40446 | 1 | H | m-D5 | H | H | H | H | A-26 | H | H | H |
| 40447 | 1 | H | m-D5 | H | H | H | H | A-27 | H | H | H |
| 40448 | 1 | H | m-D5 | H | H | H | H | A-28 | H | H | H |
| 40449 | 1 | H | m-D5 | H | H | H | H | A-29 | H | H | H |
| 40450 | 1 | H | m-D5 | H | H | H | H | A-30 | H | H | H |
| 40451 | 1 | H | m-D5 | H | H | H | H | A-31 | H | H | H |
| 40452 | 1 | H | m-D5 | H | H | H | H | A-32 | H | H | H |
| 40453 | 1 | H | m-D5 | H | H | H | H | A-33 | H | H | H |
| 40454 | 1 | H | m-D5 | H | H | H | H | A-34 | H | H | H |
| 40455 | 1 | H | m-D5 | H | H | H | H | A-35 | H | H | H |
| 40456 | 1 | H | m-D5 | H | H | H | H | A-36 | H | H | H |
| 40457 | 1 | H | m-D5 | H | H | H | H | A-37 | H | H | H |
| 40458 | 1 | H | m-D5 | H | H | H | H | A-38 | H | H | H |
| 40459 | 1 | H | m-D5 | H | H | H | H | A-39 | H | H | H |
| 40460 | 1 | H | m-D5 | H | H | H | H | A-40 | H | H | H |
| 40461 | 1 | H | m-D5 | H | H | H | H | A-41 | H | H | H |
| 40462 | 1 | H | m-D5 | H | H | H | H | A-42 | H | H | H |
| 40463 | 1 | H | m-D5 | H | H | H | H | A-43 | H | H | H |
| 40464 | 1 | H | m-D5 | H | H | H | H | A-44 | H | H | H |
| 40465 | 1 | H | m-D5 | H | H | H | H | A-45 | H | H | H |
| 40466 | 1 | H | m-D5 | H | H | H | H | A-46 | H | H | H |
| 40467 | 1 | H | m-D5 | H | H | H | H | A-47 | H | H | H |
| 40468 | 1 | H | m-D6 | H | H | H | H | A-1 | H | H | H |
| 40469 | 1 | H | m-D6 | H | H | H | H | A-2 | H | H | H |
| 40470 | 1 | H | m-D6 | H | H | H | H | A-3 | H | H | H |
| 40471 | 1 | H | m-D6 | H | H | H | H | A-4 | H | H | H |
| 40472 | 1 | H | m-D6 | H | H | H | H | A-5 | H | H | H |
| 40473 | 1 | H | m-D6 | H | H | H | H | A-6 | H | H | H |
| 40474 | 1 | H | m-D6 | H | H | H | H | A-7 | H | H | H |
| 40475 | 1 | H | m-D6 | H | H | H | H | A-8 | H | H | H |
| 40476 | 1 | H | m-D6 | H | H | H | H | A-9 | H | H | H |
| 40477 | 1 | H | m-D6 | H | H | H | H | A-10 | H | H | H |
| 40478 | 1 | H | m-D6 | H | H | H | H | A-11 | H | H | H |
| 40479 | 1 | H | m-D6 | H | H | H | H | A-12 | H | H | H |
| 40480 | 1 | H | m-D6 | H | H | H | H | A-13 | H | H | H |
| 40481 | 1 | H | m-D6 | H | H | H | H | A-14 | H | H | H |
| 40482 | 1 | H | m-D6 | H | H | H | H | A-15 | H | H | H |
| 40483 | 1 | H | m-D6 | H | H | H | H | A-16 | H | H | H |
| 40484 | 1 | H | m-D6 | H | H | H | H | A-17 | H | H | H |
| 40485 | 1 | H | m-D6 | H | H | H | H | A-18 | H | H | H |
| 40486 | 1 | H | m-D6 | H | H | H | H | A-19 | H | H | H |
| 40487 | 1 | H | m-D6 | H | H | H | H | A-20 | H | H | H |
| 40488 | 1 | H | m-D6 | H | H | H | H | A-21 | H | H | H |
| 40489 | 1 | H | m-D6 | H | H | H | H | A-22 | H | H | H |
| 40490 | 1 | H | m-D6 | H | H | H | H | A-23 | H | H | H |
| 40491 | 1 | H | m-D6 | H | H | H | H | A-24 | H | H | H |
| 40492 | 1 | H | m-D6 | H | H | H | H | A-25 | H | H | H |
| 40493 | 1 | H | m-D6 | H | H | H | H | A-26 | H | H | H |
| 40494 | 1 | H | m-D6 | H | H | H | H | A-27 | H | H | H |
| 40495 | 1 | H | m-D6 | H | H | H | H | A-28 | H | H | H |
| 40496 | 1 | H | m-D6 | H | H | H | H | A-29 | H | H | H |
| 40497 | 1 | H | m-D6 | H | H | H | H | A-30 | H | H | H |
| 40498 | 1 | H | m-D6 | H | H | H | H | A-31 | H | H | H |
| 40499 | 1 | H | m-D6 | H | H | H | H | A-32 | H | H | H |
| 40500 | 1 | H | m-D6 | H | H | H | H | A-33 | H | H | H |
| 40501 | 1 | H | m-D6 | H | H | H | H | A-34 | H | H | H |
| 40502 | 1 | H | m-D6 | H | H | H | H | A-35 | H | H | H |
| 40503 | 1 | H | m-D6 | H | H | H | H | A-36 | H | H | H |
| 40504 | 1 | H | m-D6 | H | H | H | H | A-37 | H | H | H |
| 40505 | 1 | H | m-D6 | H | H | H | H | A-38 | H | H | H |
| 40506 | 1 | H | m-D6 | H | H | H | H | A-39 | H | H | H |
| 40507 | 1 | H | m-D6 | H | H | H | H | A-40 | H | H | H |
| 40508 | 1 | H | m-D6 | H | H | H | H | A-41 | H | H | H |
| 40509 | 1 | H | m-D6 | H | H | H | H | A-42 | H | H | H |
| 40510 | 1 | H | m-D6 | H | H | H | H | A-43 | H | H | H |
| 40511 | 1 | H | m-D6 | H | H | H | H | A-44 | H | H | H |
| 40512 | 1 | H | m-D6 | H | H | H | H | A-45 | H | H | H |
| 40513 | 1 | H | m-D6 | H | H | H | H | A-46 | H | H | H |
| 40514 | 1 | H | m-D6 | H | H | H | H | A-47 | H | H | H |
| 40515 | 1 | H | m-D7 | H | H | H | H | A-1 | H | H | H |
| 40516 | 1 | H | m-D7 | H | H | H | H | A-2 | H | H | H |
| 40517 | 1 | H | m-D7 | H | H | H | H | A-3 | H | H | H |
| 40518 | 1 | H | m-D7 | H | H | H | H | A-4 | H | H | H |
| 40519 | 1 | H | m-D7 | H | H | H | H | A-5 | H | H | H |
| 40520 | 1 | H | m-D7 | H | H | H | H | A-6 | H | H | H |
| 40521 | 1 | H | m-D7 | H | H | H | H | A-7 | H | H | H |
| 40522 | 1 | H | m-D7 | H | H | H | H | A-8 | H | H | H |
| 40523 | 1 | H | m-D7 | H | H | H | H | A-9 | H | H | H |
| 40524 | 1 | H | m-D7 | H | H | H | H | A-10 | H | H | H |
| 40525 | 1 | H | m-D7 | H | H | H | H | A-11 | H | H | H |
| 40526 | 1 | H | m-D7 | H | H | H | H | A-12 | H | H | H |
| 40527 | 1 | H | m-D7 | H | H | H | H | A-13 | H | H | H |
| 40528 | 1 | H | m-D7 | H | H | H | H | A-14 | H | H | H |
| 40529 | 1 | H | m-D7 | H | H | H | H | A-15 | H | H | H |
| 40530 | 1 | H | m-D7 | H | H | H | H | A-16 | H | H | H |
| 40531 | 1 | H | m-D7 | H | H | H | H | A-17 | H | H | H |
| 40532 | 1 | H | m-D7 | H | H | H | H | A-18 | H | H | H |
| 40533 | 1 | H | m-D7 | H | H | H | H | A-19 | H | H | H |
| 40534 | 1 | H | m-D7 | H | H | H | H | A-20 | H | H | H |
| 40535 | 1 | H | m-D7 | H | H | H | H | A-21 | H | H | H |
| 40536 | 1 | H | m-D7 | H | H | H | H | A-22 | H | H | H |
| 40537 | 1 | H | m-D7 | H | H | H | H | A-23 | H | H | H |
| 40538 | 1 | H | m-D7 | H | H | H | H | A-24 | H | H | H |
| 40539 | 1 | H | m-D7 | H | H | H | H | A-25 | H | H | H |
| 40540 | 1 | H | m-D7 | H | H | H | H | A-26 | H | H | H |
| 40541 | 1 | H | m-D7 | H | H | H | H | A-27 | H | H | H |
| 40542 | 1 | H | m-D7 | H | H | H | H | A-28 | H | H | H |
| 40543 | 1 | H | m-D7 | H | H | H | H | A-29 | H | H | H |
| 40544 | 1 | H | m-D/ | H | H | H | H | A-30 | H | H | H |
| 40545 | 1 | H | m-D7 | H | H | H | H | A-31 | H | H | H |
| 40546 | 1 | H | m-D7 | H | H | H | H | A-32 | H | H | H |
| 40547 | 1 | H | m-D7 | H | H | H | H | A-33 | H | H | H |
| 40548 | 1 | H | m-D7 | H | H | H | H | A-34 | H | H | H |
| 40549 | 1 | H | m-D7 | H | H | H | H | A-35 | H | H | H |
| 40550 | 1 | H | m-D7 | H | H | H | H | A-36 | H | H | H |
| 40551 | 1 | H | m-D7 | H | H | H | H | A-37 | H | H | H |
| 40552 | 1 | H | m-D7 | H | H | H | H | A-38 | H | H | H |
| 40553 | 1 | H | m-D7 | H | H | H | H | A-39 | H | H | H |
| 40554 | 1 | H | m-D7 | H | H | H | H | A-40 | H | H | H |
| 40555 | 1 | H | m-D7 | H | H | H | H | A-41 | H | H | H |
| 40556 | 1 | H | m-D7 | H | H | H | H | A-42 | H | H | H |
| 40557 | 1 | H | m-D7 | H | H | H | H | A-43 | H | H | H |
| 40558 | 1 | H | m-D7 | H | H | H | H | A-44 | H | H | H |
| 40559 | 1 | H | m-D7 | H | H | H | H | A-45 | H | H | H |
| 40560 | 1 | H | m-D7 | H | H | H | H | A-46 | H | H | H |
| 40561 | 1 | H | m-D7 | H | H | H | H | A-47 | H | H | H |
| 40562 | 1 | H | m-D8 | H | H | H | H | A-1 | H | H | H |
| 40563 | 1 | H | m-D8 | H | H | H | H | A-2 | H | H | H |
| 40564 | 1 | H | m-D8 | H | H | H | H | A-3 | H | H | H |
| 40565 | 1 | H | m-D8 | H | H | H | H | A-4 | H | H | H |
| 40566 | 1 | H | m-D8 | H | H | H | H | A-5 | H | H | H |
| 40567 | 1 | H | m-D8 | H | H | H | H | A-6 | H | H | H |
| 40568 | 1 | H | m-D8 | H | H | H | H | A-7 | H | H | H |
| 40569 | 1 | H | m-D8 | H | H | H | H | A-8 | H | H | H |
| 40570 | 1 | H | m-D8 | H | H | H | H | A-9 | H | H | H |
| 40571 | 1 | H | m-D8 | H | H | H | H | A-10 | H | H | H |
| 40572 | 1 | H | m-D8 | H | H | H | H | A-11 | H | H | H |
| 40573 | 1 | H | m-D8 | H | H | H | H | A-12 | H | H | H |
| 40574 | 1 | H | m-D8 | H | H | H | H | A-13 | H | H | H |
| 40575 | 1 | H | m-D8 | H | H | H | H | A-14 | H | H | H |
| 40576 | 1 | H | m-D8 | H | H | H | H | A-15 | H | H | H |
| 40577 | 1 | H | m-D8 | H | H | H | H | A-16 | H | H | H |
| 40578 | 1 | H | m-D8 | H | H | H | H | A-17 | H | H | H |
| 40579 | 1 | H | m-D8 | H | H | H | H | A-18 | H | H | H |
| 40580 | 1 | H | m-D8 | H | H | H | H | A-19 | H | H | H |
| 40581 | 1 | H | m-D8 | H | H | H | H | A-20 | H | H | H |
| 40582 | 1 | H | m-D8 | H | H | H | H | A-21 | H | H | H |
| 40583 | 1 | H | m-D8 | H | H | H | H | A-22 | H | H | H |
| 40584 | 1 | H | m-D8 | H | H | H | H | A-23 | H | H | H |
| 40585 | 1 | H | m-D8 | H | H | H | H | A-24 | H | H | H |
| 40586 | 1 | H | m-D8 | H | H | H | H | A-25 | H | H | H |
| 40587 | 1 | H | m-D8 | H | H | H | H | A-26 | H | H | H |
| 40588 | 1 | H | m-D8 | H | H | H | H | A-27 | H | H | H |
| 40589 | 1 | H | m-D8 | H | H | H | H | A-28 | H | H | H |
| 40590 | 1 | H | m-D8 | H | H | H | H | A-29 | H | H | H |
| 40591 | 1 | H | m-D8 | H | H | H | H | A-30 | H | H | H |
| 40592 | 1 | H | m-D8 | H | H | H | H | A-31 | H | H | H |
| 40593 | 1 | H | m-D8 | H | H | H | H | A-32 | H | H | H |
| 40594 | 1 | H | m-D8 | H | H | H | H | A-33 | H | H | H |
| 40595 | 1 | H | m-D8 | H | H | H | H | A-34 | H | H | H |
| 40596 | 1 | H | m-D8 | H | H | H | H | A-35 | H | H | H |
| 40597 | 1 | H | m-D8 | H | H | H | H | A-36 | H | H | H |
| 40598 | 1 | H | m-D8 | H | H | H | H | A-37 | H | H | H |
| 40599 | 1 | H | m-D8 | H | H | H | H | A-38 | H | H | H |
| 40600 | 1 | H | m-D8 | H | H | H | H | A-39 | H | H | H |
| 40601 | 1 | H | m-D8 | H | H | H | H | A-40 | H | H | H |
| 40602 | 1 | H | m-D8 | H | H | H | H | A-41 | H | H | H |
| 40603 | 1 | H | m-D8 | H | H | H | H | A-42 | H | H | H |
| 40604 | 1 | H | m-D8 | H | H | H | H | A-43 | H | H | H |
| 40605 | 1 | H | m-D8 | H | H | H | H | A-44 | H | H | H |
| 40606 | 1 | H | m-D8 | H | H | H | H | A-45 | H | H | H |
| 40607 | 1 | H | m-D8 | H | H | H | H | A-46 | H | H | H |
| 40608 | 1 | H | m-D8 | H | H | H | H | A-47 | H | H | H |
| 40609 | 1 | H | m-D9 | H | H | H | H | A-1 | H | H | H |
| 40610 | 1 | H | m-D9 | H | H | H | H | A-2 | H | H | H |
| 40611 | 1 | H | m-D9 | H | H | H | H | A-3 | H | H | H |
| 40612 | 1 | H | m-D9 | H | H | H | H | A-4 | H | H | H |
| 40613 | 1 | H | m-D9 | H | H | H | H | A-5 | H | H | H |
| 40614 | 1 | H | m-D9 | H | H | H | H | A-6 | H | H | H |
| 40615 | 1 | H | m-D9 | H | H | H | H | A-7 | H | H | H |
| 40616 | 1 | H | m-D9 | H | H | H | H | A-8 | H | H | H |
| 40617 | 1 | H | m-D9 | H | H | H | H | A-9 | H | H | H |
| 40618 | 1 | H | m-D9 | H | H | H | H | A-10 | H | H | H |
| 40619 | 1 | H | m-D9 | H | H | H | H | A-11 | H | H | H |
| 40620 | 1 | H | m-D9 | H | H | H | H | A-12 | H | H | H |
| 40621 | 1 | H | m-D9 | H | H | H | H | A-13 | H | H | H |
| 40622 | 1 | H | m-D9 | H | H | H | H | A-14 | H | H | H |
| 40623 | 1 | H | m-D9 | H | H | H | H | A-15 | H | H | H |
| 40624 | 1 | H | m-D9 | H | H | H | H | A-16 | H | H | H |
| 40625 | 1 | H | m-D9 | H | H | H | H | A-17 | H | H | H |
| 40626 | 1 | H | m-D9 | H | H | H | H | A-18 | H | H | H |
| 40627 | 1 | H | m-D9 | H | H | H | H | A-19 | H | H | H |
| 40628 | 1 | H | m-D9 | H | H | H | H | A-20 | H | H | H |
| 40629 | 1 | H | m-D9 | H | H | H | H | A-21 | H | H | H |
| 40630 | 1 | H | m-D9 | H | H | H | H | A-22 | H | H | H |
| 40631 | 1 | H | m-D9 | H | H | H | H | A-23 | H | H | H |
| 40632 | 1 | H | m-D9 | H | H | H | H | A-24 | H | H | H |
| 40633 | 1 | H | m-D9 | H | H | H | H | A-25 | H | H | H |
| 40634 | 1 | H | m-D9 | H | H | H | H | A-26 | H | H | H |
| 40635 | 1 | H | m-D9 | H | H | H | H | A-27 | H | H | H |
| 40636 | 1 | H | m-D9 | H | H | H | H | A-28 | H | H | H |
| 40637 | 1 | H | m-D9 | H | H | H | H | A-29 | H | H | H |
| 40638 | 1 | H | m-D9 | H | H | H | H | A-30 | H | H | H |
| 40639 | 1 | H | m-D9 | H | H | H | H | A-31 | H | H | H |
| 40640 | 1 | H | m-D9 | H | H | H | H | A-32 | H | H | H |
| 40641 | 1 | H | m-D9 | H | H | H | H | A-33 | H | H | H |
| 40642 | 1 | H | m-D9 | H | H | H | H | A-34 | H | H | H |
| 40643 | 1 | H | m-D9 | H | H | H | H | A-35 | H | H | H |
| 40644 | 1 | H | m-D9 | H | H | H | H | A-36 | H | H | H |
| 40645 | 1 | H | m-D9 | H | H | H | H | A-37 | H | H | H |
| 40646 | 1 | H | m-D9 | H | H | H | H | A-38 | H | H | H |
| 40647 | 1 | H | m-D9 | H | H | H | H | A-39 | H | H | H |
| 40648 | 1 | H | m-D9 | H | H | H | H | A-40 | H | H | H |
| 40649 | 1 | H | m-D9 | H | H | H | H | A-41 | H | H | H |
| 40650 | 1 | H | m-D9 | H | H | H | H | A-42 | H | H | H |
| 40651 | 1 | H | m-D9 | H | H | H | H | A-43 | H | H | H |
| 40652 | 1 | H | m-D9 | H | H | H | H | A-44 | H | H | H |
| 40653 | 1 | H | m-D9 | H | H | H | H | A-45 | H | H | H |
| 40654 | 1 | H | m-D9 | H | H | H | H | A-46 | H | H | H |
| 40655 | 1 | H | m-D9 | H | H | H | H | A-47 | H | H | H |
| 40656 | 1 | H | m-D10 | H | H | H | H | A-1 | H | H | H |
| 40657 | 1 | H | m-D10 | H | H | H | H | A-2 | H | H | H |
| 40658 | 1 | H | m-D10 | H | H | H | H | A-3 | H | H | H |
| 40659 | 1 | H | m-D10 | H | H | H | H | A-4 | H | H | H |
| 40660 | 1 | H | m-D10 | H | H | H | H | A-5 | H | H | H |
| 40661 | 1 | H | m-D10 | H | H | H | H | A-6 | H | H | H |
| 40662 | 1 | H | m-D10 | H | H | H | H | A-7 | H | H | H |
| 40663 | 1 | H | m-D10 | H | H | H | H | A-8 | H | H | H |
| 40664 | 1 | H | m-D10 | H | H | H | H | A-9 | H | H | H |
| 40665 | 1 | H | m-D10 | H | H | H | H | A-10 | H | H | H |
| 40666 | 1 | H | m-D10 | H | H | H | H | A-11 | H | H | H |
| 40667 | 1 | H | m-D10 | H | H | H | H | A-12 | H | H | H |
| 40668 | 1 | H | m-D10 | H | H | H | H | A-13 | H | H | H |
| 40669 | 1 | H | m-D10 | H | H | H | H | A-14 | H | H | H |
| 40670 | 1 | H | m-D10 | H | H | H | H | A-15 | H | H | H |
| 40671 | 1 | H | m-D10 | H | H | H | H | A-16 | H | H | H |
| 40672 | 1 | H | m-D10 | H | H | H | H | A-17 | H | H | H |
| 40673 | 1 | H | m-D10 | H | H | H | H | A-18 | H | H | H |
| 40674 | 1 | H | m-D10 | H | H | H | H | A-19 | H | H | H |
| 40675 | 1 | H | m-D10 | H | H | H | H | A-20 | H | H | H |
| 40676 | 1 | H | m-D10 | H | H | H | H | A-21 | H | H | H |
| 40677 | 1 | H | m-D10 | H | H | H | H | A-22 | H | H | H |
| 40678 | 1 | H | m-D10 | H | H | H | H | A-23 | H | H | H |
| 40679 | 1 | H | m-D10 | H | H | H | H | A-24 | H | H | H |
| 40680 | 1 | H | m-D10 | H | H | H | H | A-25 | H | H | H |
| 40681 | 1 | H | m-D10 | H | H | H | H | A-26 | H | H | H |
| 40682 | 1 | H | m-D10 | H | H | H | H | A-27 | H | H | H |
| 40683 | 1 | H | m-D10 | H | H | H | H | A-28 | H | H | H |
| 40684 | 1 | H | m-D10 | H | H | H | H | A-29 | H | H | H |
| 40685 | 1 | H | m-D10 | H | H | H | H | A-30 | H | H | H |
| 40686 | 1 | H | m-D10 | H | H | H | H | A-31 | H | H | H |
| 40687 | 1 | H | m-D10 | H | H | H | H | A-32 | H | H | H |
| 40688 | 1 | H | m-D10 | H | H | H | H | A-33 | H | H | H |
| 40689 | 1 | H | m-D10 | H | H | H | H | A-34 | H | H | H |
| 40690 | 1 | H | m-D10 | H | H | H | H | A-35 | H | H | H |
| 40691 | 1 | H | m-D10 | H | H | H | H | A-36 | H | H | H |
| 40692 | 1 | H | m-D10 | H | H | H | H | A-37 | H | H | H |
| 40693 | 1 | H | m-D10 | H | H | H | H | A-38 | H | H | H |
| 40694 | 1 | H | m-D10 | H | H | H | H | A-39 | H | H | H |
| 40695 | 1 | H | m-D10 | H | H | H | H | A-40 | H | H | H |
| 40696 | 1 | H | m-D10 | H | H | H | H | A-41 | H | H | H |
| 40697 | 1 | H | m-D10 | H | H | H | H | A-42 | H | H | H |
| 40698 | 1 | H | m-D10 | H | H | H | H | A-43 | H | H | H |
| 40699 | 1 | H | m-D10 | H | H | H | H | A-44 | H | H | H |
| 40700 | 1 | H | m-D10 | H | H | H | H | A-45 | H | H | H |
| 40701 | 1 | H | m-D11 | H | H | H | H | A-46 | H | H | H |
| 40702 | 1 | H | m-D11 | H | H | H | H | A-47 | H | H | H |
| 40703 | 1 | H | m-D11 | H | H | H | H | A-1 | H | H | H |
| 40704 | 1 | H | m-D11 | H | H | H | H | A-2 | H | H | H |
| 40705 | 1 | H | m-D11 | H | H | H | H | A-3 | H | H | H |
| 40706 | 1 | H | m-D11 | H | H | H | H | A-4 | H | H | H |
| 40707 | 1 | H | m-D11 | H | H | H | H | A-5 | H | H | H |
| 40708 | 1 | H | m-D11 | H | H | H | H | A-6 | H | H | H |
| 40709 | 1 | H | m-D11 | H | H | H | H | A-7 | H | H | H |
| 40710 | 1 | H | m-D11 | H | H | H | H | A-8 | H | H | H |
| 40711 | 1 | H | m-D11 | H | H | H | H | A-9 | H | H | H |
| 40712 | 1 | H | m-D11 | H | H | H | H | A-10 | H | H | H |
| 40713 | 1 | H | m-D11 | H | H | H | H | A-11 | H | H | H |
| 40714 | 1 | H | m-D11 | H | H | H | H | A-12 | H | H | H |
| 40715 | 1 | H | m-D11 | H | H | H | H | A-13 | H | H | H |
| 40716 | 1 | H | m-D11 | H | H | H | H | A-14 | H | H | H |
| 40717 | 1 | H | m-D11 | H | H | H | H | A-15 | H | H | H |
| 40718 | 1 | H | m-D11 | H | H | H | H | A-16 | H | H | H |
| 40719 | 1 | H | m-D11 | H | H | H | H | A-17 | H | H | H |
| 40720 | 1 | H | m-D11 | H | H | H | H | A-18 | H | H | H |
| 40721 | 1 | H | m-D11 | H | H | H | H | A-19 | H | H | H |
| 40722 | 1 | H | m-D11 | H | H | H | H | A-20 | H | H | H |
| 40723 | 1 | H | m-D11 | H | H | H | H | A-21 | H | H | H |
| 40724 | 1 | H | m-D11 | H | H | H | H | A-22 | H | H | H |
| 40725 | 1 | H | m-D11 | H | H | H | H | A-23 | H | H | H |
| 40726 | 1 | H | m-D11 | H | H | H | H | A-24 | H | H | H |
| 40727 | 1 | H | m-D11 | H | H | H | H | A-25 | H | H | H |
| 40728 | 1 | H | m-D11 | H | H | H | H | A-26 | H | H | H |
| 40729 | 1 | H | m-D11 | H | H | H | H | A-27 | H | H | H |
| 40730 | 1 | H | m-D11 | H | H | H | H | A-28 | H | H | H |
| 40731 | 1 | H | m-D11 | H | H | H | H | A-29 | H | H | H |
| 40732 | 1 | H | m-D11 | H | H | H | H | A-30 | H | H | H |
| 40733 | 1 | H | m-D11 | H | H | H | H | A-31 | H | H | H |
| 40734 | 1 | H | m-D11 | H | H | H | H | A-32 | H | H | H |
| 40735 | 1 | H | m-D11 | H | H | H | H | A-33 | H | H | H |
| 40736 | 1 | H | m-D11 | H | H | H | H | A-34 | H | H | H |
| 40737 | 1 | H | m-D11 | H | H | H | H | A-35 | H | H | H |
| 40738 | 1 | H | m-D11 | H | H | H | H | A-36 | H | H | H |
| 40739 | 1 | H | m-D11 | H | H | H | H | A-37 | H | H | H |
| 40740 | 1 | H | m-D11 | H | H | H | H | A-38 | H | H | H |
| 40741 | 1 | H | m-D11 | H | H | H | H | A-39 | H | H | H |
| 40742 | 1 | H | m-D11 | H | H | H | H | A-40 | H | H | H |
| 40743 | 1 | H | m-D11 | H | H | H | H | A-41 | H | H | H |
| 40744 | 1 | H | m-D11 | H | H | H | H | A-42 | H | H | H |
| 40745 | 1 | H | m-D11 | H | H | H | H | A-43 | H | H | H |
| 40746 | 1 | H | m-D11 | H | H | H | H | A-44 | H | H | H |
| 40747 | 1 | H | m-D11 | H | H | H | H | A-45 | H | H | H |
| 40748 | 1 | H | m-D11 | H | H | H | H | A-46 | H | H | H |
| 40749 | 1 | H | m-D11 | H | H | H | H | A-47 | H | H | H |
| 40750 | 1 | H | m-D12 | H | H | H | H | A-1 | H | H | H |
| 40751 | 1 | H | m-D12 | H | H | H | H | A-2 | H | H | H |
| 40752 | 1 | H | m-D12 | H | H | H | H | A-3 | H | H | H |
| 40753 | 1 | H | m-D12 | H | H | H | H | A-4 | H | H | H |
| 40754 | 1 | H | m-D12 | H | H | H | H | A-5 | H | H | H |
| 40755 | 1 | H | m-D12 | H | H | H | H | A-6 | H | H | H |
| 40756 | 1 | H | m-D12 | H | H | H | H | A-7 | H | H | H |
| 40757 | 1 | H | m-D12 | H | H | H | H | A-8 | H | H | H |
| 40758 | 1 | H | m-D12 | H | H | H | H | A-9 | H | H | H |
| 40759 | 1 | H | m-D12 | H | H | H | H | A-10 | H | H | H |
| 40760 | 1 | H | m-D12 | H | H | H | H | A-11 | H | H | H |
| 40761 | 1 | H | m-D12 | H | H | H | H | A-12 | H | H | H |
| 40762 | 1 | H | m-D12 | H | H | H | H | A-13 | H | H | H |
| 40763 | 1 | H | m-D12 | H | H | H | H | A-14 | H | H | H |
| 40764 | 1 | H | m-D12 | H | H | H | H | A-15 | H | H | H |
| 40765 | 1 | H | m-D12 | H | H | H | H | A-16 | H | H | H |
| 40766 | 1 | H | m-D12 | H | H | H | H | A-17 | H | H | H |
| 40767 | 1 | H | m-D12 | H | H | H | H | A-18 | H | H | H |
| 40768 | 1 | H | m-D12 | H | H | H | H | A-19 | H | H | H |
| 40769 | 1 | H | m-D12 | H | H | H | H | A-20 | H | H | H |
| 40770 | 1 | H | m-D12 | H | H | H | H | A-21 | H | H | H |
| 40771 | 1 | H | m-D12 | H | H | H | H | A-22 | H | H | H |
| 40772 | 1 | H | m-D12 | H | H | H | H | A-23 | H | H | H |
| 40773 | 1 | H | m-D12 | H | H | H | H | A-24 | H | H | H |
| 40774 | 1 | H | m-D12 | H | H | H | H | A-25 | H | H | H |
| 40775 | 1 | H | m-D12 | H | H | H | H | A-26 | H | H | H |
| 40776 | 1 | H | m-D12 | H | H | H | H | A-27 | H | H | H |
| 40777 | 1 | H | m-D12 | H | H | H | H | A-28 | H | H | H |
| 40778 | 1 | H | m-D12 | H | H | H | H | A-29 | H | H | H |
| 40779 | 1 | H | m-D12 | H | H | H | H | A-30 | H | H | H |
| 40780 | 1 | H | m-D12 | H | H | H | H | A-31 | H | H | H |
| 40781 | 1 | H | m-D12 | H | H | H | H | A-32 | H | H | H |
| 40782 | 1 | H | m-D12 | H | H | H | H | A-33 | H | H | H |
| 40783 | 1 | H | m-D12 | H | H | H | H | A-34 | H | H | H |
| 40784 | 1 | H | m-D12 | H | H | H | H | A-35 | H | H | H |
| 40785 | 1 | H | m-D12 | H | H | H | H | A-36 | H | H | H |
| 40786 | 1 | H | m-D12 | H | H | H | H | A-37 | H | H | H |
| 40787 | 1 | H | m-D12 | H | H | H | H | A-38 | H | H | H |
| 40788 | 1 | H | m-D12 | H | H | H | H | A-39 | H | H | H |
| 40789 | 1 | H | m-D12 | H | H | H | H | A-40 | H | H | H |
| 40790 | 1 | H | m-D12 | H | H | H | H | A-41 | H | H | H |
| 40791 | 1 | H | m-D12 | H | H | H | H | A-42 | H | H | H |
| 40792 | 1 | H | m-D12 | H | H | H | H | A-43 | H | H | H |
| 40793 | 1 | H | m-D12 | H | H | H | H | A-44 | H | H | H |
| 40794 | 1 | H | m-D12 | H | H | H | H | A-45 | H | H | H |
| 40795 | 1 | H | m-D12 | H | H | H | H | A-46 | H | H | H |
| 40796 | 1 | H | m-D12 | H | H | H | H | A-47 | H | H | H |
| 40797 | 1 | H | m-D13 | H | H | H | H | A-1 | H | H | H |
| 40798 | 1 | H | m-D13 | H | H | H | H | A-2 | H | H | H |
| 40799 | 1 | H | m-D13 | H | H | H | H | A-3 | H | H | H |
| 40800 | 1 | H | m-D13 | H | H | H | H | A-4 | H | H | H |
| 40801 | 1 | H | m-D13 | H | H | H | H | A-5 | H | H | H |
| 40802 | 1 | H | m-D13 | H | H | H | H | A-6 | H | H | H |
| 40803 | 1 | H | m-D13 | H | H | H | H | A-7 | H | H | H |
| 40804 | 1 | H | m-D13 | H | H | H | H | A-8 | H | H | H |
| 40805 | 1 | H | m-D13 | H | H | H | H | A-9 | H | H | H |
| 40806 | 1 | H | m-D13 | H | H | H | H | A-10 | H | H | H |
| 40807 | 1 | H | m-D13 | H | H | H | H | A-11 | H | H | H |
| 40808 | 1 | H | m-D13 | H | H | H | H | A-12 | H | H | H |
| 40809 | 1 | H | m-D13 | H | H | H | H | A-13 | H | H | H |
| 40810 | 1 | H | m-D13 | H | H | H | H | A-14 | H | H | H |
| 40811 | 1 | H | m-D13 | H | H | H | H | A-15 | H | H | H |
| 40812 | 1 | H | m-D13 | H | H | H | H | A-16 | H | H | H |
| 40813 | 1 | H | m-D13 | H | H | H | H | A-17 | H | H | H |
| 40814 | 1 | H | m-D13 | H | H | H | H | A-18 | H | H | H |
| 40815 | 1 | H | m-D13 | H | H | H | H | A-19 | H | H | H |
| 40816 | 1 | H | m-D13 | H | H | H | H | A-20 | H | H | H |
| 40817 | 1 | H | m-D13 | H | H | H | H | A-21 | H | H | H |
| 40818 | 1 | H | m-D13 | H | H | H | H | A-22 | H | H | H |
| 40819 | 1 | H | m-D13 | H | H | H | H | A-23 | H | H | H |
| 40820 | 1 | H | m-D13 | H | H | H | H | A-24 | H | H | H |
| 40821 | 1 | H | m-D13 | H | H | H | H | A-25 | H | H | H |
| 40822 | 1 | H | m-D13 | H | H | H | H | A-26 | H | H | H |
| 40823 | 1 | H | m-D13 | H | H | H | H | A-27 | H | H | H |
| 40824 | 1 | H | m-D13 | H | H | H | H | A-28 | H | H | H |
| 40825 | 1 | H | m-D13 | H | H | H | H | A-29 | H | H | H |
| 40826 | 1 | H | m-D13 | H | H | H | H | A-30 | H | H | H |
| 40827 | 1 | H | m-D13 | H | H | H | H | A-31 | H | H | H |
| 40828 | 1 | H | m-D13 | H | H | H | H | A-32 | H | H | H |
| 40829 | 1 | H | m-D13 | H | H | H | H | A-33 | H | H | H |
| 40830 | 1 | H | m-D13 | H | H | H | H | A-34 | H | H | H |
| 40831 | 1 | H | m-D13 | H | H | H | H | A-35 | H | H | H |
| 40832 | 1 | H | m-D13 | H | H | H | H | A-36 | H | H | H |
| 40833 | 1 | H | m-D13 | H | H | H | H | A-37 | H | H | H |
| 40834 | 1 | H | m-D13 | H | H | H | H | A-38 | H | H | H |
| 40835 | 1 | H | m-D13 | H | H | H | H | A-39 | H | H | H |
| 40836 | 1 | H | m-D13 | H | H | H | H | A-40 | H | H | H |
| 40837 | 1 | H | m-D13 | H | H | H | H | A-41 | H | H | H |
| 40838 | 1 | H | m-D13 | H | H | H | H | A-42 | H | H | H |
| 40839 | 1 | H | m-D13 | H | H | H | H | A-43 | H | H | H |
| 40840 | 1 | H | m-D13 | H | H | H | H | A-44 | H | H | H |
| 40841 | 1 | H | m-D13 | H | H | H | H | A-45 | H | H | H |
| 40842 | 1 | H | m-D13 | H | H | H | H | A-46 | H | H | H |
| 40843 | 1 | H | m-D13 | H | H | H | H | A-47 | H | H | H |
| 40844 | 1 | H | m-D14 | H | H | H | H | A-1 | H | H | H |
| 40845 | 1 | H | m-D14 | H | H | H | H | A-2 | H | H | H |
| 40846 | 1 | H | m-D14 | H | H | H | H | A-3 | H | H | H |
| 40847 | 1 | H | m-D14 | H | H | H | H | A-4 | H | H | H |
| 40848 | 1 | H | m-D14 | H | H | H | H | A-5 | H | H | H |
| 40849 | 1 | H | m-D14 | H | H | H | H | A-6 | H | H | H |
| 40850 | 1 | H | m-D14 | H | H | H | H | A-7 | H | H | H |
| 40851 | 1 | H | m-D14 | H | H | H | H | A-8 | H | H | H |
| 40852 | 1 | H | m-D14 | H | H | H | H | A-9 | H | H | H |
| 40853 | 1 | H | m-D14 | H | H | H | H | A-10 | H | H | H |
| 40854 | 1 | H | m-D14 | H | H | H | H | A-11 | H | H | H |
| 40855 | 1 | H | m-D14 | H | H | H | H | A-12 | H | H | H |
| 40856 | 1 | H | m-D14 | H | H | H | H | A-13 | H | H | H |
| 40857 | 1 | H | m-D14 | H | H | H | H | A-14 | H | H | H |
| 40858 | 1 | H | m-D14 | H | H | H | H | A-15 | H | H | H |
| 40859 | 1 | H | m-D14 | H | H | H | H | A-16 | H | H | H |
| 40860 | 1 | H | m-D14 | H | H | H | H | A-17 | H | H | H |
| 40861 | 1 | H | m-D14 | H | H | H | H | A-18 | H | H | H |
| 40862 | 1 | H | m-D14 | H | H | H | H | A-19 | H | H | H |
| 40863 | 1 | H | m-D14 | H | H | H | H | A-20 | H | H | H |
| 40864 | 1 | H | m-D14 | H | H | H | H | A-21 | H | H | H |
| 40865 | 1 | H | m-D14 | H | H | H | H | A-22 | H | H | H |
| 40866 | 1 | H | m-D14 | H | H | H | H | A-23 | H | H | H |
| 40867 | 1 | H | m-D14 | H | H | H | H | A-24 | H | H | H |
| 40868 | 1 | H | m-D14 | H | H | H | H | A-25 | H | H | H |
| 40869 | 1 | H | m-D14 | H | H | H | H | A-26 | H | H | H |
| 40870 | 1 | H | m-D14 | H | H | H | H | A-27 | H | H | H |
| 40871 | 1 | H | m-D14 | H | H | H | H | A-28 | H | H | H |
| 40872 | 1 | H | m-D14 | H | H | H | H | A-29 | H | H | H |
| 40873 | 1 | H | m-D14 | H | H | H | H | A-30 | H | H | H |
| 40874 | 1 | H | m-D14 | H | H | H | H | A-31 | H | H | H |
| 40875 | 1 | H | m-D14 | H | H | H | H | A-32 | H | H | H |
| 40876 | 1 | H | m-D14 | H | H | H | H | A-33 | H | H | H |
| 40877 | 1 | H | m-D14 | H | H | H | H | A-34 | H | H | H |
| 40878 | 1 | H | m-D14 | H | H | H | H | A-35 | H | H | H |
| 40879 | 1 | H | m-D14 | H | H | H | H | A-36 | H | H | H |
| 40880 | 1 | H | m-D14 | H | H | H | H | A-37 | H | H | H |
| 40881 | 1 | H | m-D14 | H | H | H | H | A-38 | H | H | H |
| 40882 | 1 | H | m-D14 | H | H | H | H | A-39 | H | H | H |
| 40883 | 1 | H | m-D14 | H | H | H | H | A-40 | H | H | H |
| 40884 | 1 | H | m-D14 | H | H | H | H | A-41 | H | H | H |
| 40885 | 1 | H | m-D14 | H | H | H | H | A-42 | H | H | H |
| 40886 | 1 | H | m-D14 | H | H | H | H | A-43 | H | H | H |
| 40887 | 1 | H | m-D14 | H | H | H | H | A-44 | H | H | H |
| 40888 | 1 | H | m-D14 | H | H | H | H | A-45 | H | H | H |
| 40889 | 1 | H | m-D14 | H | H | H | H | A-46 | H | H | H |
| 40890 | 1 | H | m-D14 | H | H | H | H | A-47 | H | H | H |
| 40891 | 1 | m-D1 | H | H | H | H | A-1 | H | H | H | H |
| 40892 | 1 | m-D1 | H | H | H | H | A-2 | H | H | H | H |
| 40893 | 1 | m-D1 | H | H | H | H | A-3 | H | H | H | H |
| 40894 | 1 | m-D1 | H | H | H | H | A-4 | H | H | H | H |
| 40895 | 1 | m-D1 | H | H | H | H | A-5 | H | H | H | H |
| 40896 | 1 | m-D1 | H | H | H | H | A-6 | H | H | H | H |
| 40897 | 1 | m-D1 | H | H | H | H | A-7 | H | H | H | H |
| 40898 | 1 | m-D1 | H | H | H | H | A-8 | H | H | H | H |
| 40899 | 1 | m-D1 | H | H | H | H | A-9 | H | H | H | H |
| 40900 | 1 | m-D1 | H | H | H | H | A-10 | H | H | H | H |
| 40901 | 1 | m-D1 | H | H | H | H | A-11 | H | H | H | H |
| 40902 | 1 | m-D1 | H | H | H | H | A-12 | H | H | H | H |
| 40903 | 1 | m-D1 | H | H | H | H | A-13 | H | H | H | H |
| 40904 | 1 | m-D1 | H | H | H | H | A-14 | H | H | H | H |
| 40905 | 1 | m-D1 | H | H | H | H | A-15 | H | H | H | H |
| 40906 | 1 | m-D1 | H | H | H | H | A-16 | H | H | H | H |
| 40907 | 1 | m-D1 | H | H | H | H | A-17 | H | H | H | H |
| 40908 | 1 | m-D1 | H | H | H | H | A-18 | H | H | H | H |
| 40909 | 1 | m-D1 | H | H | H | H | A-19 | H | H | H | H |
| 40910 | 1 | m-D1 | H | H | H | H | A-20 | H | H | H | H |
| 40911 | 1 | m-D1 | H | H | H | H | A-21 | H | H | H | H |
| 40912 | 1 | m-D1 | H | H | H | H | A-22 | H | H | H | H |
| 40913 | 1 | m-D1 | H | H | H | H | A-23 | H | H | H | H |
| 40914 | 1 | m-D1 | H | H | H | H | A-24 | H | H | H | H |
| 40915 | 1 | m-D1 | H | H | H | H | A-25 | H | H | H | H |
| 40916 | 1 | m-D1 | H | H | H | H | A-26 | H | H | H | H |
| 40917 | 1 | m-D1 | H | H | H | H | A-27 | H | H | H | H |
| 40918 | 1 | m-D1 | H | H | H | H | A-28 | H | H | H | H |
| 40919 | 1 | m-D1 | H | H | H | H | A-29 | H | H | H | H |
| 40920 | 1 | m-D1 | H | H | H | H | A-30 | H | H | H | H |
| 40921 | 1 | m-D1 | H | H | H | H | A-31 | H | H | H | H |
| 40922 | 1 | m-D1 | H | H | H | H | A-32 | H | H | H | H |
| 40923 | 1 | m-D1 | H | H | H | H | A-33 | H | H | H | H |
| 40924 | 1 | m-D1 | H | H | H | H | A-34 | H | H | H | H |
| 40925 | 1 | m-D1 | H | H | H | H | A-35 | H | H | H | H |
| 40926 | 1 | m-D1 | H | H | H | H | A-36 | H | H | H | H |
| 40927 | 1 | m-D1 | H | H | H | H | A-37 | H | H | H | H |
| 40928 | 1 | m-D1 | H | H | H | H | A-38 | H | H | H | H |
| 40929 | 1 | m-D1 | H | H | H | H | A-39 | H | H | H | H |
| 40930 | 1 | m-D1 | H | H | H | H | A-40 | H | H | H | H |
| 40931 | 1 | m-D1 | H | H | H | H | A-41 | H | H | H | H |
| 40932 | 1 | m-D1 | H | H | H | H | A-42 | H | H | H | H |
| 40933 | 1 | m-D1 | H | H | H | H | A-43 | H | H | H | H |
| 40934 | 1 | m-D1 | H | H | H | H | A-44 | H | H | H | H |
| 40935 | 1 | m-D1 | H | H | H | H | A-45 | H | H | H | H |
| 40936 | 1 | m-D1 | H | H | H | H | A-46 | H | H | H | H |
| 40937 | 1 | m-D1 | H | H | H | H | A-47 | H | H | H | H |
| 40938 | 1 | m-D2 | H | H | H | H | A-1 | H | H | H | H |
| 40939 | 1 | m-D2 | H | H | H | H | A-2 | H | H | H | H |
| 40940 | 1 | m-D2 | H | H | H | H | A-3 | H | H | H | H |
| 40941 | 1 | m-D2 | H | H | H | H | A-4 | H | H | H | H |
| 40942 | 1 | m-D2 | H | H | H | H | A-5 | H | H | H | H |
| 40943 | 1 | m-D2 | H | H | H | H | A-6 | H | H | H | H |
| 40944 | 1 | m-D2 | H | H | H | H | A-7 | H | H | H | H |
| 40945 | 1 | m-D2 | H | H | H | H | A-8 | H | H | H | H |
| 40946 | 1 | m-D2 | H | H | H | H | A-9 | H | H | H | H |
| 40947 | 1 | m-D2 | H | H | H | H | A-10 | H | H | H | H |
| 40948 | 1 | m-D2 | H | H | H | H | A-11 | H | H | H | H |
| 40949 | 1 | m-D2 | H | H | H | H | A-12 | H | H | H | H |
| 40950 | 1 | m-D2 | H | H | H | H | A-13 | H | H | H | H |
| 40951 | 1 | m-D2 | H | H | H | H | A-14 | H | H | H | H |
| 40952 | 1 | m-D2 | H | H | H | H | A-15 | H | H | H | H |
| 40953 | 1 | m-D2 | H | H | H | H | A-16 | H | H | H | H |
| 40954 | 1 | m-D2 | H | H | H | H | A-17 | H | H | H | H |
| 40955 | 1 | m-D2 | H | H | H | H | A-18 | H | H | H | H |
| 40956 | 1 | m-D2 | H | H | H | H | A-19 | H | H | H | H |
| 40957 | 1 | m-D2 | H | H | H | H | A-20 | H | H | H | H |
| 40958 | 1 | m-D2 | H | H | H | H | A-21 | H | H | H | H |
| 40959 | 1 | m-D2 | H | H | H | H | A-22 | H | H | H | H |
| 40960 | 1 | m-D2 | H | H | H | H | A-23 | H | H | H | H |
| 40961 | 1 | m-D2 | H | H | H | H | A-24 | H | H | H | H |
| 40962 | 1 | m-D2 | H | H | H | H | A-25 | H | H | H | H |
| 40963 | 1 | m-D2 | H | H | H | H | A-26 | H | H | H | H |
| 40964 | 1 | m-D2 | H | H | H | H | A-27 | H | H | H | H |
| 40965 | 1 | m-D2 | H | H | H | H | A-28 | H | H | H | H |
| 40966 | 1 | m-D2 | H | H | H | H | A-29 | H | H | H | H |
| 40967 | 1 | m-D2 | H | H | H | H | A-30 | H | H | H | H |
| 40968 | 1 | m-D2 | H | H | H | H | A-31 | H | H | H | H |
| 40969 | 1 | m-D2 | H | H | H | H | A-32 | H | H | H | H |
| 40970 | 1 | m-D2 | H | H | H | H | A-33 | H | H | H | H |
| 40971 | 1 | m-D2 | H | H | H | H | A-34 | H | H | H | H |
| 40972 | 1 | m-D2 | H | H | H | H | A-35 | H | H | H | H |
| 40973 | 1 | m-D2 | H | H | H | H | A-36 | H | H | H | H |
| 40974 | 1 | m-D2 | H | H | H | H | A-37 | H | H | H | H |
| 40975 | 1 | m-D2 | H | H | H | H | A-38 | H | H | H | H |
| 40976 | 1 | m-D2 | H | H | H | H | A-39 | H | H | H | H |
| 40977 | 1 | m-D2 | H | H | H | H | A-40 | H | H | H | H |
| 40978 | 1 | m-D2 | H | H | H | H | A-41 | H | H | H | H |
| 40979 | 1 | m-D2 | H | H | H | H | A-42 | H | H | H | H |
| 40980 | 1 | m-D2 | H | H | H | H | A-43 | H | H | H | H |
| 40981 | 1 | m-D2 | H | H | H | H | A-44 | H | H | H | H |
| 40982 | 1 | m-D2 | H | H | H | H | A-45 | H | H | H | H |
| 40983 | 1 | m-D2 | H | H | H | H | A-46 | H | H | H | H |
| 40984 | 1 | m-D2 | H | H | H | H | A-47 | H | H | H | H |
| 40985 | 1 | m-D3 | H | H | H | H | A-1 | H | H | H | H |
| 40986 | 1 | m-D3 | H | H | H | H | A-2 | H | H | H | H |
| 40987 | 1 | m-D3 | H | H | H | H | A-3 | H | H | H | H |
| 40988 | 1 | m-D3 | H | H | H | H | A-4 | H | H | H | H |
| 40989 | 1 | m-D3 | H | H | H | H | A-5 | H | H | H | H |
| 40990 | 1 | m-D3 | H | H | H | H | A-6 | H | H | H | H |
| 40991 | 1 | m-D3 | H | H | H | H | A-7 | H | H | H | H |
| 40992 | 1 | m-D3 | H | H | H | H | A-8 | H | H | H | H |
| 40993 | 1 | m-D3 | H | H | H | H | A-9 | H | H | H | H |
| 40994 | 1 | m-D3 | H | H | H | H | A-10 | H | H | H | H |
| 40995 | 1 | m-D3 | H | H | H | H | A-11 | H | H | H | H |
| 40996 | 1 | m-D3 | H | H | H | H | A-12 | H | H | H | H |
| 40997 | 1 | m-D3 | H | H | H | H | A-13 | H | H | H | H |
| 40998 | 1 | m-D3 | H | H | H | H | A-14 | H | H | H | H |
| 40999 | 1 | m-D3 | H | H | H | H | A-15 | H | H | H | H |
| 41000 | 1 | m-D3 | H | H | H | H | A-16 | H | H | H | H |
| 41001 | 1 | m-D3 | H | H | H | H | A-17 | H | H | H | H |
| 41002 | 1 | m-D3 | H | H | H | H | A-18 | H | H | H | H |
| 41003 | 1 | m-D3 | H | H | H | H | A-19 | H | H | H | H |
| 41004 | 1 | m-D3 | H | H | H | H | A-20 | H | H | H | H |
| 41005 | 1 | m-D3 | H | H | H | H | A-21 | H | H | H | H |
| 41006 | 1 | m-D3 | H | H | H | H | A-22 | H | H | H | H |
| 41007 | 1 | m-D3 | H | H | H | H | A-23 | H | H | H | H |
| 41008 | 1 | m-D3 | H | H | H | H | A-24 | H | H | H | H |
| 41009 | 1 | m-D3 | H | H | H | H | A-25 | H | H | H | H |
| 41010 | 1 | m-D3 | H | H | H | H | A-26 | H | H | H | H |
| 41011 | 1 | m-D3 | H | H | H | H | A-27 | H | H | H | H |
| 41012 | 1 | m-D3 | H | H | H | H | A-28 | H | H | H | H |
| 41013 | 1 | m-D3 | H | H | H | H | A-29 | H | H | H | H |
| 41014 | 1 | m-D3 | H | H | H | H | A-30 | H | H | H | H |
| 41015 | 1 | m-D3 | H | H | H | H | A-31 | H | H | H | H |
| 41016 | 1 | m-D3 | H | H | H | H | A-32 | H | H | H | H |
| 41017 | 1 | m-D3 | H | H | H | H | A-33 | H | H | H | H |
| 41018 | 1 | m-D3 | H | H | H | H | A-34 | H | H | H | H |
| 41019 | 1 | m-D3 | H | H | H | H | A-35 | H | H | H | H |
| 41020 | 1 | m-D3 | H | H | H | H | A-36 | H | H | H | H |
| 41021 | 1 | m-D3 | H | H | H | H | A-37 | H | H | H | H |
| 41022 | 1 | m-D3 | H | H | H | H | A-38 | H | H | H | H |
| 41023 | 1 | m-D3 | H | H | H | H | A-39 | H | H | H | H |
| 41024 | 1 | m-D3 | H | H | H | H | A-40 | H | H | H | H |
| 41025 | 1 | m-D3 | H | H | H | H | A-41 | H | H | H | H |
| 41026 | 1 | m-D3 | H | H | H | H | A-42 | H | H | H | H |
| 41027 | 1 | m-D3 | H | H | H | H | A-43 | H | H | H | H |
| 41028 | 1 | m-D3 | H | H | H | H | A-44 | H | H | H | H |
| 41029 | 1 | m-D3 | H | H | H | H | A-45 | H | H | H | H |
| 41030 | 1 | m-D3 | H | H | H | H | A-46 | H | H | H | H |
| 41031 | 1 | m-D3 | H | H | H | H | A-47 | H | H | H | H |
| 41032 | 1 | m-D4 | H | H | H | H | A-1 | H | H | H | H |
| 41033 | 1 | m-D4 | H | H | H | H | A-2 | H | H | H | H |
| 41034 | 1 | m-D4 | H | H | H | H | A-3 | H | H | H | H |
| 41035 | 1 | m-D4 | H | H | H | H | A-4 | H | H | H | H |
| 41036 | 1 | m-D4 | H | H | H | H | A-5 | H | H | H | H |
| 41037 | 1 | m-D4 | H | H | H | H | A-6 | H | H | H | H |
| 41038 | 1 | m-D4 | H | H | H | H | A-7 | H | H | H | H |
| 41039 | 1 | m-D4 | H | H | H | H | A-8 | H | H | H | H |
| 41040 | 1 | m-D4 | H | H | H | H | A-9 | H | H | H | H |
| 41041 | 1 | m-D4 | H | H | H | H | A-10 | H | H | H | H |
| 41042 | 1 | m-D4 | H | H | H | H | A-11 | H | H | H | H |
| 41043 | 1 | m-D4 | H | H | H | H | A-12 | H | H | H | H |
| 41044 | 1 | m-D4 | H | H | H | H | A-13 | H | H | H | H |
| 41045 | 1 | m-D4 | H | H | H | H | A-14 | H | H | H | H |
| 41046 | 1 | m-D4 | H | H | H | H | A-15 | H | H | H | H |
| 41047 | 1 | m-D4 | H | H | H | H | A-16 | H | H | H | H |
| 41048 | 1 | m-D4 | H | H | H | H | A-17 | H | H | H | H |
| 41049 | 1 | m-D4 | H | H | H | H | A-18 | H | H | H | H |
| 41050 | 1 | m-D4 | H | H | H | H | A-19 | H | H | H | H |
| 41051 | 1 | m-D4 | H | H | H | H | A-20 | H | H | H | H |
| 41052 | 1 | m-D4 | H | H | H | H | A-21 | H | H | H | H |
| 41053 | 1 | m-D4 | H | H | H | H | A-22 | H | H | H | H |
| 41054 | 1 | m-D4 | H | H | H | H | A-23 | H | H | H | H |
| 41055 | 1 | m-D4 | H | H | H | H | A-24 | H | H | H | H |
| 41056 | 1 | m-D4 | H | H | H | H | A-25 | H | H | H | H |
| 41057 | 1 | m-D4 | H | H | H | H | A-26 | H | H | H | H |
| 41058 | 1 | m-D4 | H | H | H | H | A-27 | H | H | H | H |
| 41059 | 1 | m-D4 | H | H | H | H | A-28 | H | H | H | H |
| 41060 | 1 | m-D4 | H | H | H | H | A-29 | H | H | H | H |
| 41061 | 1 | m-D4 | H | H | H | H | A-30 | H | H | H | H |
| 41062 | 1 | m-D4 | H | H | H | H | A-31 | H | H | H | H |
| 41063 | 1 | m-D4 | H | H | H | H | A-32 | H | H | H | H |
| 41064 | 1 | m-D4 | H | H | H | H | A-33 | H | H | H | H |
| 41065 | 1 | m-D4 | H | H | H | H | A-34 | H | H | H | H |
| 41066 | 1 | m-D4 | H | H | H | H | A-35 | H | H | H | H |
| 41067 | 1 | m-D4 | H | H | H | H | A-36 | H | H | H | H |
| 41068 | 1 | m-D4 | H | H | H | H | A-37 | H | H | H | H |
| 41069 | 1 | m-D4 | H | H | H | H | A-38 | H | H | H | H |
| 41070 | 1 | m-D4 | H | H | H | H | A-39 | H | H | H | H |
| 41071 | 1 | m-D4 | H | H | H | H | A-40 | H | H | H | H |
| 41072 | 1 | m-D4 | H | H | H | H | A-41 | H | H | H | H |
| 41073 | 1 | m-D4 | H | H | H | H | A-42 | H | H | H | H |
| 41074 | 1 | m-D4 | H | H | H | H | A-43 | H | H | H | H |
| 41075 | 1 | m-D4 | H | H | H | H | A-44 | H | H | H | H |
| 41076 | 1 | m-D4 | H | H | H | H | A-45 | H | H | H | H |
| 41077 | 1 | m-D4 | H | H | H | H | A-46 | H | H | H | H |
| 41078 | 1 | m-D4 | H | H | H | H | A-47 | H | H | H | H |
| 41079 | 1 | m-D5 | H | H | H | H | A-1 | H | H | H | H |
| 41080 | 1 | m-D5 | H | H | H | H | A-2 | H | H | H | H |
| 41081 | 1 | m-D5 | H | H | H | H | A-3 | H | H | H | H |
| 41082 | 1 | m-D5 | H | H | H | H | A-4 | H | H | H | H |
| 41083 | 1 | m-D5 | H | H | H | H | A-5 | H | H | H | H |
| 41084 | 1 | m-D5 | H | H | H | H | A-6 | H | H | H | H |
| 41085 | 1 | m-D5 | H | H | H | H | A-7 | H | H | H | H |
| 41086 | 1 | m-D5 | H | H | H | H | A-8 | H | H | H | H |
| 41087 | 1 | m-D5 | H | H | H | H | A-9 | H | H | H | H |
| 41088 | 1 | m-D5 | H | H | H | H | A-10 | H | H | H | H |
| 41089 | 1 | m-D5 | H | H | H | H | A-11 | H | H | H | H |
| 41090 | 1 | m-D5 | H | H | H | H | A-12 | H | H | H | H |
| 41091 | 1 | m-D5 | H | H | H | H | A-13 | H | H | H | H |
| 41092 | 1 | m-D5 | H | H | H | H | A-14 | H | H | H | H |
| 41093 | 1 | m-D5 | H | H | H | H | A-15 | H | H | H | H |
| 41094 | 1 | m-D5 | H | H | H | H | A-16 | H | H | H | H |
| 41095 | 1 | m-D5 | H | H | H | H | A-17 | H | H | H | H |
| 41096 | 1 | m-D5 | H | H | H | H | A-18 | H | H | H | H |
| 41097 | 1 | m-D5 | H | H | H | H | A-19 | H | H | H | H |
| 41098 | 1 | m-D5 | H | H | H | H | A-20 | H | H | H | H |
| 41099 | 1 | m-D5 | H | H | H | H | A-21 | H | H | H | H |
| 41100 | 1 | m-D5 | H | H | H | H | A-22 | H | H | H | H |
| 41101 | 1 | m-D5 | H | H | H | H | A-23 | H | H | H | H |
| 41102 | 1 | m-D5 | H | H | H | H | A-24 | H | H | H | H |
| 41103 | 1 | m-D5 | H | H | H | H | A-25 | H | H | H | H |
| 41104 | 1 | m-D5 | H | H | H | H | A-26 | H | H | H | H |
| 41105 | 1 | m-D5 | H | H | H | H | A-27 | H | H | H | H |
| 41106 | 1 | m-D5 | H | H | H | H | A-28 | H | H | H | H |
| 41107 | 1 | m-D5 | H | H | H | H | A-29 | H | H | H | H |
| 41108 | 1 | m-D5 | H | H | H | H | A-30 | H | H | H | H |
| 41109 | 1 | m-D5 | H | H | H | H | A-31 | H | H | H | H |
| 41110 | 1 | m-D5 | H | H | H | H | A-32 | H | H | H | H |
| 41111 | 1 | m-D5 | H | H | H | H | A-33 | H | H | H | H |
| 41112 | 1 | m-D5 | H | H | H | H | A-34 | H | H | H | H |
| 41113 | 1 | m-D5 | H | H | H | H | A-35 | H | H | H | H |
| 41114 | 1 | m-D5 | H | H | H | H | A-36 | H | H | H | H |
| 41115 | 1 | m-D5 | H | H | H | H | A-37 | H | H | H | H |
| 41116 | 1 | m-D5 | H | H | H | H | A-38 | H | H | H | H |
| 41117 | 1 | m-D5 | H | H | H | H | A-39 | H | H | H | H |
| 41118 | 1 | m-D5 | H | H | H | H | A-40 | H | H | H | H |
| 41119 | 1 | m-D5 | H | H | H | H | A-41 | H | H | H | H |
| 41120 | 1 | m-D5 | H | H | H | H | A-42 | H | H | H | H |
| 41121 | 1 | m-D5 | H | H | H | H | A-43 | H | H | H | H |
| 41122 | 1 | m-D5 | H | H | H | H | A-44 | H | H | H | H |
| 41123 | 1 | m-D5 | H | H | H | H | A-45 | H | H | H | H |
| 41124 | 1 | m-D5 | H | H | H | H | A-46 | H | H | H | H |
| 41125 | 1 | m-D5 | H | H | H | H | A-47 | H | H | H | H |
| 41126 | 1 | m-D6 | H | H | H | H | A-1 | H | H | H | H |
| 41127 | 1 | m-D6 | H | H | H | H | A-2 | H | H | H | H |
| 41128 | 1 | m-D6 | H | H | H | H | A-3 | H | H | H | H |
| 41129 | 1 | m-D6 | H | H | H | H | A-4 | H | H | H | H |
| 41130 | 1 | m-D6 | H | H | H | H | A-5 | H | H | H | H |
| 41131 | 1 | m-D6 | H | H | H | H | A-6 | H | H | H | H |
| 41132 | 1 | m-D6 | H | H | H | H | A-7 | H | H | H | H |
| 41133 | 1 | m-D6 | H | H | H | H | A-8 | H | H | H | H |
| 41134 | 1 | m-D6 | H | H | H | H | A-9 | H | H | H | H |
| 41135 | 1 | m-D6 | H | H | H | H | A-10 | H | H | H | H |
| 41136 | 1 | m-D6 | H | H | H | H | A-11 | H | H | H | H |
| 41137 | 1 | m-D6 | H | H | H | H | A-12 | H | H | H | H |
| 41138 | 1 | m-D6 | H | H | H | H | A-13 | H | H | H | H |
| 41139 | 1 | m-D6 | H | H | H | H | A-14 | H | H | H | H |
| 41140 | 1 | m-D6 | H | H | H | H | A-15 | H | H | H | H |
| 41141 | 1 | m-D6 | H | H | H | H | A-16 | H | H | H | H |
| 41142 | 1 | m-D6 | H | H | H | H | A-17 | H | H | H | H |
| 41143 | 1 | m-D6 | H | H | H | H | A-18 | H | H | H | H |
| 41144 | 1 | m-D6 | H | H | H | H | A-19 | H | H | H | H |
| 41145 | 1 | m-D6 | H | H | H | H | A-20 | H | H | H | H |
| 41146 | 1 | m-D6 | H | H | H | H | A-21 | H | H | H | H |
| 41147 | 1 | m-D6 | H | H | H | H | A-22 | H | H | H | H |
| 41148 | 1 | m-D6 | H | H | H | H | A-23 | H | H | H | H |
| 41149 | 1 | m-D6 | H | H | H | H | A-24 | H | H | H | H |
| 41150 | 1 | m-D6 | H | H | H | H | A-25 | H | H | H | H |
| 41151 | 1 | m-D6 | H | H | H | H | A-26 | H | H | H | H |
| 41152 | 1 | m-D6 | H | H | H | H | A-27 | H | H | H | H |
| 41153 | 1 | m-D6 | H | H | H | H | A-28 | H | H | H | H |
| 41154 | 1 | m-D6 | H | H | H | H | A-29 | H | H | H | H |
| 41155 | 1 | m-D6 | H | H | H | H | A-30 | H | H | H | H |
| 41156 | 1 | m-D6 | H | H | H | H | A-31 | H | H | H | H |
| 41157 | 1 | m-D6 | H | H | H | H | A-32 | H | H | H | H |
| 41158 | 1 | m-D6 | H | H | H | H | A-33 | H | H | H | H |
| 41159 | 1 | m-D6 | H | H | H | H | A-34 | H | H | H | H |
| 41160 | 1 | m-D6 | H | H | H | H | A-35 | H | H | H | H |
| 41161 | 1 | m-D6 | H | H | H | H | A-36 | H | H | H | H |
| 41162 | 1 | m-D6 | H | H | H | H | A-37 | H | H | H | H |
| 41163 | 1 | m-D6 | H | H | H | H | A-38 | H | H | H | H |
| 41164 | 1 | m-D6 | H | H | H | H | A-39 | H | H | H | H |
| 41165 | 1 | m-D6 | H | H | H | H | A-40 | H | H | H | H |
| 41166 | 1 | m-D6 | H | H | H | H | A-41 | H | H | H | H |
| 41167 | 1 | m-D6 | H | H | H | H | A-42 | H | H | H | H |
| 41168 | 1 | m-D6 | H | H | H | H | A-43 | H | H | H | H |
| 41169 | 1 | m-D6 | H | H | H | H | A-44 | H | H | H | H |
| 41170 | 1 | m-D6 | H | H | H | H | A-45 | H | H | H | H |
| 41171 | 1 | m-D6 | H | H | H | H | A-46 | H | H | H | H |
| 41172 | 1 | m-D6 | H | H | H | H | A-47 | H | H | H | H |
| 41173 | 1 | m-D7 | H | H | H | H | A-1 | H | H | H | H |
| 41174 | 1 | m-D7 | H | H | H | H | A-2 | H | H | H | H |
| 41175 | 1 | m-D7 | H | H | H | H | A-3 | H | H | H | H |
| 41176 | 1 | m-D7 | H | H | H | H | A-4 | H | H | H | H |
| 41177 | 1 | m-D7 | H | H | H | H | A-5 | H | H | H | H |
| 41178 | 1 | m-D7 | H | H | H | H | A-6 | H | H | H | H |
| 41179 | 1 | m-D7 | H | H | H | H | A-7 | H | H | H | H |
| 41180 | 1 | m-D7 | H | H | H | H | A-8 | H | H | H | H |
| 41181 | 1 | m-D7 | H | H | H | H | A-9 | H | H | H | H |
| 41182 | 1 | m-D7 | H | H | H | H | A-10 | H | H | H | H |
| 41183 | 1 | m-D7 | H | H | H | H | A-11 | H | H | H | H |
| 41184 | 1 | m-D7 | H | H | H | H | A-12 | H | H | H | H |
| 41185 | 1 | m-D7 | H | H | H | H | A-13 | H | H | H | H |
| 41186 | 1 | m-D7 | H | H | H | H | A-14 | H | H | H | H |
| 41187 | 1 | m-D7 | H | H | H | H | A-15 | H | H | H | H |
| 41188 | 1 | m-D7 | H | H | H | H | A-16 | H | H | H | H |
| 41189 | 1 | m-D7 | H | H | H | H | A-17 | H | H | H | H |
| 41190 | 1 | m-D7 | H | H | H | H | A-18 | H | H | H | H |
| 41191 | 1 | m-D7 | H | H | H | H | A-19 | H | H | H | H |
| 41192 | 1 | m-D7 | H | H | H | H | A-20 | H | H | H | H |
| 41193 | 1 | m-D7 | H | H | H | H | A-21 | H | H | H | H |
| 41194 | 1 | m-D7 | H | H | H | H | A-22 | H | H | H | H |
| 41195 | 1 | m-D7 | H | H | H | H | A-23 | H | H | H | H |
| 41196 | 1 | m-D7 | H | H | H | H | A-24 | H | H | H | H |
| 41197 | 1 | m-D7 | H | H | H | H | A-25 | H | H | H | H |
| 41198 | 1 | m-D7 | H | H | H | H | A-26 | H | H | H | H |
| 41199 | 1 | m-D7 | H | H | H | H | A-27 | H | H | H | H |
| 41200 | 1 | m-D7 | H | H | H | H | A-28 | H | H | H | H |
| 41201 | 1 | m-D7 | H | H | H | H | A-29 | H | H | H | H |
| 41202 | 1 | m-D7 | H | H | H | H | A-30 | H | H | H | H |
| 41203 | 1 | m-D7 | H | H | H | H | A-31 | H | H | H | H |
| 41204 | 1 | m-D7 | H | H | H | H | A-32 | H | H | H | H |
| 41205 | 1 | m-D7 | H | H | H | H | A-33 | H | H | H | H |
| 41206 | 1 | m-D7 | H | H | H | H | A-34 | H | H | H | H |
| 41207 | 1 | m-D7 | H | H | H | H | A-35 | H | H | H | H |
| 41208 | 1 | m-D7 | H | H | H | H | A-36 | H | H | H | H |
| 41209 | 1 | m-D7 | H | H | H | H | A-37 | H | H | H | H |
| 41210 | 1 | m-D7 | H | H | H | H | A-38 | H | H | H | H |
| 41211 | 1 | m-D7 | H | H | H | H | A-39 | H | H | H | H |
| 41212 | 1 | m-D7 | H | H | H | H | A-40 | H | H | H | H |
| 41213 | 1 | m-D7 | H | H | H | H | A-41 | H | H | H | H |
| 41214 | 1 | m-D7 | H | H | H | H | A-42 | H | H | H | H |
| 41215 | 1 | m-D7 | H | H | H | H | A-43 | H | H | H | H |
| 41216 | 1 | m-D7 | H | H | H | H | A-44 | H | H | H | H |
| 41217 | 1 | m-D7 | H | H | H | H | A-45 | H | H | H | H |
| 41218 | 1 | m-D7 | H | H | H | H | A-46 | H | H | H | H |
| 41219 | 1 | m-D7 | H | H | H | H | A-47 | H | H | H | H |
| 41220 | 1 | m-D8 | H | H | H | H | A-1 | H | H | H | H |
| 41221 | 1 | m-D8 | H | H | H | H | A-2 | H | H | H | H |
| 41222 | 1 | m-D8 | H | H | H | H | A-3 | H | H | H | H |
| 41223 | 1 | m-D8 | H | H | H | H | A-4 | H | H | H | H |
| 41224 | 1 | m-D8 | H | H | H | H | A-5 | H | H | H | H |
| 41225 | 1 | m-D8 | H | H | H | H | A-6 | H | H | H | H |
| 41226 | 1 | m-D8 | H | H | H | H | A-7 | H | H | H | H |
| 41227 | 1 | m-D8 | H | H | H | H | A-8 | H | H | H | H |
| 41228 | 1 | m-D8 | H | H | H | H | A-9 | H | H | H | H |
| 41229 | 1 | m-D8 | H | H | H | H | A-10 | H | H | H | H |
| 41230 | 1 | m-D8 | H | H | H | H | A-11 | H | H | H | H |
| 41231 | 1 | m-D8 | H | H | H | H | A-12 | H | H | H | H |
| 41232 | 1 | m-D8 | H | H | H | H | A-13 | H | H | H | H |
| 41233 | 1 | m-D8 | H | H | H | H | A-14 | H | H | H | H |
| 41234 | 1 | m-D8 | H | H | H | H | A-15 | H | H | H | H |
| 41235 | 1 | m-D8 | H | H | H | H | A-16 | H | H | H | H |
| 41236 | 1 | m-D8 | H | H | H | H | A-17 | H | H | H | H |
| 41237 | 1 | m-D8 | H | H | H | H | A-18 | H | H | H | H |
| 41238 | 1 | m-D8 | H | H | H | H | A-19 | H | H | H | H |
| 41239 | 1 | m-D8 | H | H | H | H | A-20 | H | H | H | H |
| 41240 | 1 | m-D8 | H | H | H | H | A-21 | H | H | H | H |
| 41241 | 1 | m-D8 | H | H | H | H | A-22 | H | H | H | H |
| 41242 | 1 | m-D8 | H | H | H | H | A-23 | H | H | H | H |
| 41243 | 1 | m-D8 | H | H | H | H | A-24 | H | H | H | H |
| 41244 | 1 | m-D8 | H | H | H | H | A-25 | H | H | H | H |
| 41245 | 1 | m-D8 | H | H | H | H | A-26 | H | H | H | H |
| 41246 | 1 | m-D8 | H | H | H | H | A-27 | H | H | H | H |
| 41247 | 1 | m-D8 | H | H | H | H | A-28 | H | H | H | H |
| 41248 | 1 | m-D8 | H | H | H | H | A-29 | H | H | H | H |
| 41249 | 1 | m-D8 | H | H | H | H | A-30 | H | H | H | H |
| 41250 | 1 | m-D8 | H | H | H | H | A-31 | H | H | H | H |
| 41251 | 1 | m-D8 | H | H | H | H | A-32 | H | H | H | H |
| 41252 | 1 | m-D8 | H | H | H | H | A-33 | H | H | H | H |
| 41253 | 1 | m-D8 | H | H | H | H | A-34 | H | H | H | H |
| 41254 | 1 | m-D8 | H | H | H | H | A-35 | H | H | H | H |
| 41255 | 1 | m-D8 | H | H | H | H | A-36 | H | H | H | H |
| 41256 | 1 | m-D8 | H | H | H | H | A-37 | H | H | H | H |
| 41257 | 1 | m-D8 | H | H | H | H | A-38 | H | H | H | H |
| 41258 | 1 | m-D8 | H | H | H | H | A-39 | H | H | H | H |
| 41259 | 1 | m-D8 | H | H | H | H | A-40 | H | H | H | H |
| 41260 | 1 | m-D8 | H | H | H | H | A-41 | H | H | H | H |
| 41261 | 1 | m-D8 | H | H | H | H | A-42 | H | H | H | H |
| 41262 | 1 | m-D8 | H | H | H | H | A-43 | H | H | H | H |
| 41263 | 1 | m-D8 | H | H | H | H | A-44 | H | H | H | H |
| 41264 | 1 | m-D8 | H | H | H | H | A-45 | H | H | H | H |
| 41265 | 1 | m-D8 | H | H | H | H | A-46 | H | H | H | H |
| 41266 | 1 | m-D8 | H | H | H | H | A-47 | H | H | H | H |
| 41267 | 1 | m-D9 | H | H | H | H | A-1 | H | H | H | H |
| 41268 | 1 | m-D9 | H | H | H | H | A-2 | H | H | H | H |
| 41269 | 1 | m-D9 | H | H | H | H | A-3 | H | H | H | H |
| 41270 | 1 | m-D9 | H | H | H | H | A-4 | H | H | H | H |
| 41271 | 1 | m-D9 | H | H | H | H | A-5 | H | H | H | H |
| 41272 | 1 | m-D9 | H | H | H | H | A-6 | H | H | H | H |
| 41273 | 1 | m-D9 | H | H | H | H | A-7 | H | H | H | H |
| 41274 | 1 | m-D9 | H | H | H | H | A-8 | H | H | H | H |
| 41275 | 1 | m-D9 | H | H | H | H | A-9 | H | H | H | H |
| 41276 | 1 | m-D9 | H | H | H | H | A-10 | H | H | H | H |
| 41277 | 1 | m-D9 | H | H | H | H | A-11 | H | H | H | H |
| 41278 | 1 | m-D9 | H | H | H | H | A-12 | H | H | H | H |
| 41279 | 1 | m-D9 | H | H | H | H | A-13 | H | H | H | H |
| 41280 | 1 | m-D9 | H | H | H | H | A-14 | H | H | H | H |
| 41281 | 1 | m-D9 | H | H | H | H | A-15 | H | H | H | H |
| 41282 | 1 | m-D9 | H | H | H | H | A-16 | H | H | H | H |
| 41283 | 1 | m-D9 | H | H | H | H | A-17 | H | H | H | H |
| 41284 | 1 | m-D9 | H | H | H | H | A-18 | H | H | H | H |
| 41285 | 1 | m-D9 | H | H | H | H | A-19 | H | H | H | H |
| 41286 | 1 | m-D9 | H | H | H | H | A-20 | H | H | H | H |
| 41287 | 1 | m-D9 | H | H | H | H | A-21 | H | H | H | H |
| 41288 | 1 | m-D9 | H | H | H | H | A-22 | H | H | H | H |
| 41289 | 1 | m-D9 | H | H | H | H | A-23 | H | H | H | H |
| 41290 | 1 | m-D9 | H | H | H | H | A-24 | H | H | H | H |
| 41291 | 1 | m-D9 | H | H | H | H | A-25 | H | H | H | H |
| 41292 | 1 | m-D9 | H | H | H | H | A-26 | H | H | H | H |
| 41293 | 1 | m-D9 | H | H | H | H | A-27 | H | H | H | H |
| 41294 | 1 | m-D9 | H | H | H | H | A-28 | H | H | H | H |
| 41295 | 1 | m-D9 | H | H | H | H | A-29 | H | H | H | H |
| 41296 | 1 | m-D9 | H | H | H | H | A-30 | H | H | H | H |
| 41297 | 1 | m-D9 | H | H | H | H | A-31 | H | H | H | H |
| 41298 | 1 | m-D9 | H | H | H | H | A-32 | H | H | H | H |
| 41299 | 1 | m-D9 | H | H | H | H | A-33 | H | H | H | H |
| 41300 | 1 | m-D9 | H | H | H | H | A-34 | H | H | H | H |
| 41301 | 1 | m-D9 | H | H | H | H | A-35 | H | H | H | H |
| 41302 | 1 | m-D9 | H | H | H | H | A-36 | H | H | H | H |
| 41303 | 1 | m-D9 | H | H | H | H | A-37 | H | H | H | H |
| 41304 | 1 | m-D9 | H | H | H | H | A-38 | H | H | H | H |
| 41305 | 1 | m-D9 | H | H | H | H | A-39 | H | H | H | H |
| 41306 | 1 | m-D9 | H | H | H | H | A-40 | H | H | H | H |
| 41307 | 1 | m-D9 | H | H | H | H | A-41 | H | H | H | H |
| 41308 | 1 | m-D9 | H | H | H | H | A-42 | H | H | H | H |
| 41309 | 1 | m-D9 | H | H | H | H | A-43 | H | H | H | H |
| 41310 | 1 | m-D9 | H | H | H | H | A-44 | H | H | H | H |
| 41311 | 1 | m-D9 | H | H | H | H | A-45 | H | H | H | H |
| 41312 | 1 | m-D9 | H | H | H | H | A-46 | H | H | H | H |
| 41313 | 1 | m-D9 | H | H | H | H | A-47 | H | H | H | H |
| 41314 | 1 | m-D10 | H | H | H | H | A-1 | H | H | H | H |
| 41315 | 1 | m-D10 | H | H | H | H | A-2 | H | H | H | H |
| 41316 | 1 | m-D10 | H | H | H | H | A-3 | H | H | H | H |
| 41317 | 1 | m-D10 | H | H | H | H | A-4 | H | H | H | H |
| 41318 | 1 | m-D10 | H | H | H | H | A-5 | H | H | H | H |
| 41319 | 1 | m-D10 | H | H | H | H | A-6 | H | H | H | H |
| 41320 | 1 | m-D10 | H | H | H | H | A-7 | H | H | H | H |
| 41321 | 1 | m-D10 | H | H | H | H | A-8 | H | H | H | H |
| 41322 | 1 | m-D10 | H | H | H | H | A-9 | H | H | H | H |
| 41323 | 1 | m-D10 | H | H | H | H | A-10 | H | H | H | H |
| 41324 | 1 | m-D10 | H | H | H | H | A-11 | H | H | H | H |
| 41325 | 1 | m-D10 | H | H | H | H | A-12 | H | H | H | H |
| 41326 | 1 | m-D10 | H | H | H | H | A-13 | H | H | H | H |
| 41327 | 1 | m-D10 | H | H | H | H | A-14 | H | H | H | H |
| 41328 | 1 | m-D10 | H | H | H | H | A-15 | H | H | H | H |
| 41329 | 1 | m-D10 | H | H | H | H | A-16 | H | H | H | H |
| 41330 | 1 | m-D10 | H | H | H | H | A-17 | H | H | H | H |
| 41331 | 1 | m-D10 | H | H | H | H | A-18 | H | H | H | H |
| 41332 | 1 | m-D10 | H | H | H | H | A-19 | H | H | H | H |
| 41333 | 1 | m-D10 | H | H | H | H | A-20 | H | H | H | H |
| 41334 | 1 | m-D10 | H | H | H | H | A-21 | H | H | H | H |
| 41335 | 1 | m-D10 | H | H | H | H | A-22 | H | H | H | H |
| 41336 | 1 | m-D10 | H | H | H | H | A-23 | H | H | H | H |
| 41337 | 1 | m-D10 | H | H | H | H | A-24 | H | H | H | H |
| 41338 | 1 | m-D10 | H | H | H | H | A-25 | H | H | H | H |
| 41339 | 1 | m-D10 | H | H | H | H | A-26 | H | H | H | H |
| 41340 | 1 | m-D10 | H | H | H | H | A-27 | H | H | H | H |
| 41341 | 1 | m-D10 | H | H | H | H | A-28 | H | H | H | H |
| 41342 | 1 | m-D10 | H | H | H | H | A-29 | H | H | H | H |
| 41343 | 1 | m-D10 | H | H | H | H | A-30 | H | H | H | H |
| 41344 | 1 | m-D10 | H | H | H | H | A-31 | H | H | H | H |
| 41345 | 1 | m-D10 | H | H | H | H | A-32 | H | H | H | H |
| 41346 | 1 | m-D10 | H | H | H | H | A-33 | H | H | H | H |
| 41347 | 1 | m-D10 | H | H | H | H | A-34 | H | H | H | H |
| 41348 | 1 | m-D10 | H | H | H | H | A-35 | H | H | H | H |
| 41349 | 1 | m-D10 | H | H | H | H | A-36 | H | H | H | H |
| 41350 | 1 | m-D10 | H | H | H | H | A-37 | H | H | H | H |
| 41351 | 1 | m-D10 | H | H | H | H | A-38 | H | H | H | H |
| 41352 | 1 | m-D10 | H | H | H | H | A-39 | H | H | H | H |
| 41353 | 1 | m-D10 | H | H | H | H | A-40 | H | H | H | H |
| 41354 | 1 | m-D10 | H | H | H | H | A-41 | H | H | H | H |
| 41355 | 1 | m-D10 | H | H | H | H | A-42 | H | H | H | H |
| 41356 | 1 | m-D10 | H | H | H | H | A-43 | H | H | H | H |
| 41357 | 1 | m-D10 | H | H | H | H | A-44 | H | H | H | H |
| 41358 | 1 | m-D10 | H | H | H | H | A-45 | H | H | H | H |
| 41359 | 1 | m-D10 | H | H | H | H | A-46 | H | H | H | H |
| 41360 | 1 | m-D10 | H | H | H | H | A-47 | H | H | H | H |
| 41361 | 1 | m-D11 | H | H | H | H | A-1 | H | H | H | H |
| 41362 | 1 | m-D11 | H | H | H | H | A-2 | H | H | H | H |
| 41363 | 1 | m-D11 | H | H | H | H | A-3 | H | H | H | H |
| 41364 | 1 | m-D11 | H | H | H | H | A-4 | H | H | H | H |
| 41365 | 1 | m-D11 | H | H | H | H | A-5 | H | H | H | H |
| 41366 | 1 | m-D11 | H | H | H | H | A-6 | H | H | H | H |
| 41367 | 1 | m-D11 | H | H | H | H | A-7 | H | H | H | H |
| 41368 | 1 | m-D11 | H | H | H | H | A-8 | H | H | H | H |
| 41369 | 1 | m-D11 | H | H | H | H | A-9 | H | H | H | H |
| 41370 | 1 | m-D11 | H | H | H | H | A-10 | H | H | H | H |
| 41371 | 1 | m-D11 | H | H | H | H | A-11 | H | H | H | H |
| 41372 | 1 | m-D11 | H | H | H | H | A-12 | H | H | H | H |
| 41373 | 1 | m-D11 | H | H | H | H | A-13 | H | H | H | H |
| 41374 | 1 | m-D11 | H | H | H | H | A-14 | H | H | H | H |
| 41375 | 1 | m-D11 | H | H | H | H | A-15 | H | H | H | H |
| 41376 | 1 | m-D11 | H | H | H | H | A-16 | H | H | H | H |
| 41377 | 1 | m-D11 | H | H | H | H | A-17 | H | H | H | H |
| 41378 | 1 | m-D11 | H | H | H | H | A-18 | H | H | H | H |
| 41379 | 1 | m-D11 | H | H | H | H | A-19 | H | H | H | H |
| 41380 | 1 | m-D11 | H | H | H | H | A-20 | H | H | H | H |
| 41381 | 1 | m-D11 | H | H | H | H | A-21 | H | H | H | H |
| 41382 | 1 | m-D11 | H | H | H | H | A-22 | H | H | H | H |
| 41383 | 1 | m-D11 | H | H | H | H | A-23 | H | H | H | H |
| 41384 | 1 | m-D11 | H | H | H | H | A-24 | H | H | H | H |
| 41385 | 1 | m-D11 | H | H | H | H | A-25 | H | H | H | H |
| 41386 | 1 | m-D11 | H | H | H | H | A-26 | H | H | H | H |
| 41387 | 1 | m-D11 | H | H | H | H | A-27 | H | H | H | H |
| 41388 | 1 | m-D11 | H | H | H | H | A-28 | H | H | H | H |
| 41389 | 1 | m-D11 | H | H | H | H | A-29 | H | H | H | H |
| 41390 | 1 | m-D11 | H | H | H | H | A-30 | H | H | H | H |
| 41391 | 1 | m-D11 | H | H | H | H | A-31 | H | H | H | H |
| 41392 | 1 | m-D11 | H | H | H | H | A-32 | H | H | H | H |
| 41393 | 1 | m-D11 | H | H | H | H | A-33 | H | H | H | H |
| 41394 | 1 | m-D11 | H | H | H | H | A-34 | H | H | H | H |
| 41395 | 1 | m-D11 | H | H | H | H | A-35 | H | H | H | H |
| 41396 | 1 | m-D11 | H | H | H | H | A-36 | H | H | H | H |
| 41397 | 1 | m-D11 | H | H | H | H | A-37 | H | H | H | H |
| 41398 | 1 | m-D11 | H | H | H | H | A-38 | H | H | H | H |
| 41399 | 1 | m-D11 | H | H | H | H | A-39 | H | H | H | H |
| 41400 | 1 | m-D11 | H | H | H | H | A-40 | H | H | H | H |
| 41401 | 1 | m-D11 | H | H | H | H | A-41 | H | H | H | H |
| 41402 | 1 | m-D11 | H | H | H | H | A-42 | H | H | H | H |
| 41403 | 1 | m-D11 | H | H | H | H | A-43 | H | H | H | H |
| 41404 | 1 | m-D11 | H | H | H | H | A-44 | H | H | H | H |
| 41405 | 1 | m-D11 | H | H | H | H | A-45 | H | H | H | H |
| 41406 | 1 | m-D11 | H | H | H | H | A-46 | H | H | H | H |
| 41407 | 1 | m-D11 | H | H | H | H | A-47 | H | H | H | H |
| 41408 | 1 | m-D12 | H | H | H | H | A-1 | H | H | H | H |
| 41409 | 1 | m-D12 | H | H | H | H | A-2 | H | H | H | H |
| 41410 | 1 | m-D12 | H | H | H | H | A-3 | H | H | H | H |
| 41411 | 1 | m-D12 | H | H | H | H | A-4 | H | H | H | H |
| 41412 | 1 | m-D12 | H | H | H | H | A-5 | H | H | H | H |
| 41413 | 1 | m-D12 | H | H | H | H | A-6 | H | H | H | H |
| 41414 | 1 | m-D12 | H | H | H | H | A-7 | H | H | H | H |
| 41415 | 1 | m-D12 | H | H | H | H | A-8 | H | H | H | H |
| 41416 | 1 | m-D12 | H | H | H | H | A-9 | H | H | H | H |
| 41417 | 1 | m-D12 | H | H | H | H | A-10 | H | H | H | H |
| 41418 | 1 | m-D12 | H | H | H | H | A-11 | H | H | H | H |
| 41419 | 1 | m-D12 | H | H | H | H | A-12 | H | H | H | H |
| 41420 | 1 | m-D12 | H | H | H | H | A-13 | H | H | H | H |
| 41421 | 1 | m-D12 | H | H | H | H | A-14 | H | H | H | H |
| 41422 | 1 | m-D12 | H | H | H | H | A-15 | H | H | H | H |
| 41423 | 1 | m-D12 | H | H | H | H | A-16 | H | H | H | H |
| 41424 | 1 | m-D12 | H | H | H | H | A-17 | H | H | H | H |
| 41425 | 1 | m-D12 | H | H | H | H | A-18 | H | H | H | H |
| 41426 | 1 | m-D12 | H | H | H | H | A-19 | H | H | H | H |
| 41427 | 1 | m-D12 | H | H | H | H | A-20 | H | H | H | H |
| 41428 | 1 | m-D12 | H | H | H | H | A-21 | H | H | H | H |
| 41429 | 1 | m-D12 | H | H | H | H | A-22 | H | H | H | H |
| 41430 | 1 | m-D12 | H | H | H | H | A-23 | H | H | H | H |
| 41431 | 1 | m-D12 | H | H | H | H | A-24 | H | H | H | H |
| 41432 | 1 | m-D12 | H | H | H | H | A-25 | H | H | H | H |
| 41433 | 1 | m-D12 | H | H | H | H | A-26 | H | H | H | H |
| 41434 | 1 | m-D12 | H | H | H | H | A-27 | H | H | H | H |
| 41435 | 1 | m-D12 | H | H | H | H | A-28 | H | H | H | H |
| 41436 | 1 | m-D12 | H | H | H | H | A-29 | H | H | H | H |
| 41437 | 1 | m-D12 | H | H | H | H | A-30 | H | H | H | H |
| 41438 | 1 | m-D12 | H | H | H | H | A-31 | H | H | H | H |
| 41439 | 1 | m-D12 | H | H | H | H | A-32 | H | H | H | H |
| 41440 | 1 | m-D12 | H | H | H | H | A-33 | H | H | H | H |
| 41441 | 1 | m-D12 | H | H | H | H | A-34 | H | H | H | H |
| 41442 | 1 | m-D12 | H | H | H | H | A-35 | H | H | H | H |
| 41443 | 1 | m-D12 | H | H | H | H | A-36 | H | H | H | H |
| 41444 | 1 | m-D12 | H | H | H | H | A-37 | H | H | H | H |
| 41445 | 1 | m-D12 | H | H | H | H | A-38 | H | H | H | H |
| 41446 | 1 | m-D12 | H | H | H | H | A-39 | H | H | H | H |
| 41447 | 1 | m-D12 | H | H | H | H | A-40 | H | H | H | H |
| 41448 | 1 | m-D12 | H | H | H | H | A-41 | H | H | H | H |
| 41449 | 1 | m-D12 | H | H | H | H | A-42 | H | H | H | H |
| 41450 | 1 | m-D12 | H | H | H | H | A-43 | H | H | H | H |
| 41451 | 1 | m-D12 | H | H | H | H | A-44 | H | H | H | H |
| 41452 | 1 | m-D12 | H | H | H | H | A-45 | H | H | H | H |
| 41453 | 1 | m-D12 | H | H | H | H | A-46 | H | H | H | H |
| 41454 | 1 | m-D12 | H | H | H | H | A-47 | H | H | H | H |
| 41455 | 1 | m-D13 | H | H | H | H | A-1 | H | H | H | H |
| 41456 | 1 | m-D13 | H | H | H | H | A-2 | H | H | H | H |
| 41457 | 1 | m-D13 | H | H | H | H | A-3 | H | H | H | H |
| 41458 | 1 | m-D13 | H | H | H | H | A-4 | H | H | H | H |
| 41459 | 1 | m-D13 | H | H | H | H | A-5 | H | H | H | H |
| 41460 | 1 | m-D13 | H | H | H | H | A-6 | H | H | H | H |
| 41461 | 1 | m-D13 | H | H | H | H | A-7 | H | H | H | H |
| 41462 | 1 | m-D13 | H | H | H | H | A-8 | H | H | H | H |
| 41463 | 1 | m-D13 | H | H | H | H | A-9 | H | H | H | H |
| 41464 | 1 | m-D13 | H | H | H | H | A-10 | H | H | H | H |
| 41465 | 1 | m-D13 | H | H | H | H | A-11 | H | H | H | H |
| 41466 | 1 | m-D13 | H | H | H | H | A-12 | H | H | H | H |
| 41467 | 1 | m-D13 | H | H | H | H | A-13 | H | H | H | H |
| 41468 | 1 | m-D13 | H | H | H | H | A-14 | H | H | H | H |
| 41469 | 1 | m-D13 | H | H | H | H | A-15 | H | H | H | H |
| 41470 | 1 | m-D13 | H | H | H | H | A-16 | H | H | H | H |
| 41471 | 1 | m-D13 | H | H | H | H | A-17 | H | H | H | H |
| 41472 | 1 | m-D13 | H | H | H | H | A-18 | H | H | H | H |
| 41473 | 1 | m-D13 | H | H | H | H | A-19 | H | H | H | H |
| 41474 | 1 | m-D13 | H | H | H | H | A-20 | H | H | H | H |
| 41475 | 1 | m-D13 | H | H | H | H | A-21 | H | H | H | H |
| 41476 | 1 | m-D13 | H | H | H | H | A-22 | H | H | H | H |
| 41477 | 1 | m-D13 | H | H | H | H | A-23 | H | H | H | H |
| 41478 | 1 | m-D13 | H | H | H | H | A-24 | H | H | H | H |
| 41479 | 1 | m-D13 | H | H | H | H | A-25 | H | H | H | H |
| 41480 | 1 | m-D13 | H | H | H | H | A-26 | H | H | H | H |
| 41481 | 1 | m-D13 | H | H | H | H | A-27 | H | H | H | H |
| 41482 | 1 | m-D13 | H | H | H | H | A-28 | H | H | H | H |
| 41483 | 1 | m-D13 | H | H | H | H | A-29 | H | H | H | H |
| 41484 | 1 | m-D13 | H | H | H | H | A-30 | H | H | H | H |
| 41485 | 1 | m-D13 | H | H | H | H | A-31 | H | H | H | H |
| 41486 | 1 | m-D13 | H | H | H | H | A-32 | H | H | H | H |
| 41487 | 1 | m-D13 | H | H | H | H | A-33 | H | H | H | H |
| 41488 | 1 | m-D13 | H | H | H | H | A-34 | H | H | H | H |
| 41489 | 1 | m-D13 | H | H | H | H | A-35 | H | H | H | H |
| 41490 | 1 | m-D13 | H | H | H | H | A-36 | H | H | H | H |
| 41491 | 1 | m-D13 | H | H | H | H | A-37 | H | H | H | H |
| 41492 | 1 | m-D13 | H | H | H | H | A-38 | H | H | H | H |
| 41493 | 1 | m-D13 | H | H | H | H | A-39 | H | H | H | H |
| 41494 | 1 | m-D13 | H | H | H | H | A-40 | H | H | H | H |
| 41495 | 1 | m-D13 | H | H | H | H | A-41 | H | H | H | H |
| 41496 | 1 | m-D13 | H | H | H | H | A-42 | H | H | H | H |
| 41497 | 1 | m-D13 | H | H | H | H | A-43 | H | H | H | H |
| 41498 | 1 | m-D13 | H | H | H | H | A-44 | H | H | H | H |
| 41499 | 1 | m-D13 | H | H | H | H | A-45 | H | H | H | H |
| 41500 | 1 | m-D13 | H | H | H | H | A-46 | H | H | H | H |
| 41501 | 1 | m-D13 | H | H | H | H | A-47 | H | H | H | H |
| 41502 | 1 | m-D14 | H | H | H | H | A-1 | H | H | H | H |
| 41503 | 1 | m-D14 | H | H | H | H | A-2 | H | H | H | H |
| 41504 | 1 | m-D14 | H | H | H | H | A-3 | H | H | H | H |
| 41505 | 1 | m-D14 | H | H | H | H | A-4 | H | H | H | H |
| 41506 | 1 | m-D14 | H | H | H | H | A-5 | H | H | H | H |
| 41507 | 1 | m-D14 | H | H | H | H | A-6 | H | H | H | H |
| 41508 | 1 | m-D14 | H | H | H | H | A-7 | H | H | H | H |
| 41509 | 1 | m-D14 | H | H | H | H | A-8 | H | H | H | H |
| 41510 | 1 | m-D14 | H | H | H | H | A-9 | H | H | H | H |
| 41511 | 1 | m-D14 | H | H | H | H | A-10 | H | H | H | H |
| 41512 | 1 | m-D14 | H | H | H | H | A-11 | H | H | H | H |
| 41513 | 1 | m-D14 | H | H | H | H | A-12 | H | H | H | H |
| 41514 | 1 | m-D14 | H | H | H | H | A-13 | H | H | H | H |
| 41515 | 1 | m-D14 | H | H | H | H | A-14 | H | H | H | H |
| 41516 | 1 | m-D14 | H | H | H | H | A-15 | H | H | H | H |
| 41517 | 1 | m-D14 | H | H | H | H | A-16 | H | H | H | H |
| 41518 | 1 | m-D14 | H | H | H | H | A-17 | H | H | H | H |
| 41519 | 1 | m-D14 | H | H | H | H | A-18 | H | H | H | H |
| 41520 | 1 | m-D14 | H | H | H | H | A-19 | H | H | H | H |
| 41521 | 1 | m-D14 | H | H | H | H | A-20 | H | H | H | H |
| 41522 | 1 | m-D14 | H | H | H | H | A-21 | H | H | H | H |
| 41523 | 1 | m-D14 | H | H | H | H | A-22 | H | H | H | H |
| 41524 | 1 | m-D14 | H | H | H | H | A-23 | H | H | H | H |
| 41525 | 1 | m-D14 | H | H | H | H | A-24 | H | H | H | H |
| 41526 | 1 | m-D14 | H | H | H | H | A-25 | H | H | H | H |
| 41527 | 1 | m-D14 | H | H | H | H | A-26 | H | H | H | H |
| 41528 | 1 | m-D14 | H | H | H | H | A-27 | H | H | H | H |
| 41529 | 1 | m-D14 | H | H | H | H | A-28 | H | H | H | H |
| 41530 | 1 | m-D14 | H | H | H | H | A-29 | H | H | H | H |
| 41531 | 1 | m-D14 | H | H | H | H | A-30 | H | H | H | H |
| 41532 | 1 | m-D14 | H | H | H | H | A-31 | H | H | H | H |
| 41533 | 1 | m-D14 | H | H | H | H | A-32 | H | H | H | H |
| 41534 | 1 | m-D14 | H | H | H | H | A-33 | H | H | H | H |
| 41535 | 1 | m-D14 | H | H | H | H | A-34 | H | H | H | H |
| 41536 | 1 | m-D14 | H | H | H | H | A-35 | H | H | H | H |
| 41537 | 1 | m-D14 | H | H | H | H | A-36 | H | H | H | H |
| 41538 | 1 | m-D14 | H | H | H | H | A-37 | H | H | H | H |
| 41539 | 1 | m-D14 | H | H | H | H | A-38 | H | H | H | H |
| 41540 | 1 | m-D14 | H | H | H | H | A-39 | H | H | H | H |
| 41541 | 1 | m-D14 | H | H | H | H | A-40 | H | H | H | H |
| 41542 | 1 | m-D14 | H | H | H | H | A-41 | H | H | H | H |
| 41543 | 1 | m-D14 | H | H | H | H | A-42 | H | H | H | H |
| 41544 | 1 | m-D14 | H | H | H | H | A-43 | H | H | H | H |
| 41545 | 1 | m-D14 | H | H | H | H | A-44 | H | H | H | H |
| 41546 | 1 | m-D14 | H | H | H | H | A-45 | H | H | H | H |
| 41547 | 1 | m-D14 | H | H | H | H | A-46 | H | H | H | H |
| 41548 | 1 | m-D14 | H | H | H | H | A-47 | H | H | H | H |
| 41549 | 1 | H | H | m-D1 | H | H | A-1 | H | H | H | H |
| 41550 | 1 | H | H | m-D1 | H | H | A-2 | H | H | H | H |
| 41551 | 1 | H | H | m-D1 | H | H | A-3 | H | H | H | H |
| 41552 | 1 | H | H | m-D1 | H | H | A-4 | H | H | H | H |
| 41553 | 1 | H | H | m-D1 | H | H | A-5 | H | H | H | H |
| 41554 | 1 | H | H | m-D1 | H | H | A-6 | H | H | H | H |
| 41555 | 1 | H | H | m-D1 | H | H | A-7 | H | H | H | H |
| 41556 | 1 | H | H | m-D1 | H | H | A-8 | H | H | H | H |
| 41557 | 1 | H | H | m-D1 | H | H | A-9 | H | H | H | H |
| 41558 | 1 | H | H | m-D1 | H | H | A-10 | H | H | H | H |
| 41559 | 1 | H | H | m-D1 | H | H | A-11 | H | H | H | H |
| 41560 | 1 | H | H | m-D1 | H | H | A-12 | H | H | H | H |
| 41561 | 1 | H | H | m-D1 | H | H | A-13 | H | H | H | H |
| 41562 | 1 | H | H | m-D1 | H | H | A-14 | H | H | H | H |
| 41563 | 1 | H | H | m-D1 | H | H | A-15 | H | H | H | H |
| 41564 | 1 | H | H | m-D1 | H | H | A-16 | H | H | H | H |
| 41565 | 1 | H | H | m-D1 | H | H | A-17 | H | H | H | H |
| 41566 | 1 | H | H | m-D1 | H | H | A-18 | H | H | H | H |
| 41567 | 1 | H | H | m-D1 | H | H | A-19 | H | H | H | H |
| 41568 | 1 | H | H | m-D1 | H | H | A-20 | H | H | H | H |
| 41569 | 1 | H | H | m-D1 | H | H | A-21 | H | H | H | H |
| 41570 | 1 | H | H | m-D1 | H | H | A-22 | H | H | H | H |
| 41571 | 1 | H | H | m-D1 | H | H | A-23 | H | H | H | H |
| 41572 | 1 | H | H | m-D1 | H | H | A-24 | H | H | H | H |
| 41573 | 1 | H | H | m-D1 | H | H | A-25 | H | H | H | H |
| 41574 | 1 | H | H | m-D1 | H | H | A-26 | H | H | H | H |
| 41575 | 1 | H | H | m-D1 | H | H | A-27 | H | H | H | H |
| 41576 | 1 | H | H | m-D1 | H | H | A-28 | H | H | H | H |
| 41577 | 1 | H | H | m-D1 | H | H | A-29 | H | H | H | H |
| 41578 | 1 | H | H | m-D1 | H | H | A-30 | H | H | H | H |
| 41579 | 1 | H | H | m-D1 | H | H | A-31 | H | H | H | H |
| 41580 | 1 | H | H | m-D1 | H | H | A-32 | H | H | H | H |
| 41581 | 1 | H | H | m-D1 | H | H | A-33 | H | H | H | H |
| 41582 | 1 | H | H | m-D1 | H | H | A-34 | H | H | H | H |
| 41583 | 1 | H | H | m-D1 | H | H | A-35 | H | H | H | H |
| 41584 | 1 | H | H | m-D1 | H | H | A-36 | H | H | H | H |
| 41585 | 1 | H | H | m-D1 | H | H | A-37 | H | H | H | H |
| 41586 | 1 | H | H | m-D1 | H | H | A-38 | H | H | H | H |
| 41587 | 1 | H | H | m-D1 | H | H | A-39 | H | H | H | H |
| 41588 | 1 | H | H | m-D1 | H | H | A-40 | H | H | H | H |
| 41589 | 1 | H | H | m-D1 | H | H | A-41 | H | H | H | H |
| 41590 | 1 | H | H | m-D1 | H | H | A-42 | H | H | H | H |
| 41591 | 1 | H | H | m-D1 | H | H | A-43 | H | H | H | H |
| 41592 | 1 | H | H | m-D1 | H | H | A-44 | H | H | H | H |
| 41593 | 1 | H | H | m-D1 | H | H | A-45 | H | H | H | H |
| 41594 | 1 | H | H | m-D1 | H | H | A-46 | H | H | H | H |
| 41595 | 1 | H | H | m-D1 | H | H | A-47 | H | H | H | H |
| 41596 | 1 | H | H | m-D2 | H | H | A-1 | H | H | H | H |
| 41597 | 1 | H | H | m-D2 | H | H | A-2 | H | H | H | H |
| 41598 | 1 | H | H | m-D2 | H | H | A-3 | H | H | H | H |
| 41599 | 1 | H | H | m-D2 | H | H | A-4 | H | H | H | H |
| 41600 | 1 | H | H | m-D2 | H | H | A-5 | H | H | H | H |
| 41601 | 1 | H | H | m-D2 | H | H | A-6 | H | H | H | H |
| 41602 | 1 | H | H | m-D2 | H | H | A-7 | H | H | H | H |
| 41603 | 1 | H | H | m-D2 | H | H | A-8 | H | H | H | H |
| 41604 | 1 | H | H | m-D2 | H | H | A-9 | H | H | H | H |
| 41605 | 1 | H | H | m-D2 | H | H | A-10 | H | H | H | H |
| 41606 | 1 | H | H | m-D2 | H | H | A-11 | H | H | H | H |
| 41607 | 1 | H | H | m-D2 | H | H | A-12 | H | H | H | H |
| 41608 | 1 | H | H | m-D2 | H | H | A-13 | H | H | H | H |
| 41609 | 1 | H | H | m-D2 | H | H | A-14 | H | H | H | H |
| 41610 | 1 | H | H | m-D2 | H | H | A-15 | H | H | H | H |
| 41611 | 1 | H | H | m-D2 | H | H | A-16 | H | H | H | H |
| 41612 | 1 | H | H | m-D2 | H | H | A-17 | H | H | H | H |
| 41613 | 1 | H | H | m-D2 | H | H | A-18 | H | H | H | H |
| 41614 | 1 | H | H | m-D2 | H | H | A-19 | H | H | H | H |
| 41615 | 1 | H | H | m-D2 | H | H | A-20 | H | H | H | H |
| 41616 | 1 | H | H | m-D2 | H | H | A-21 | H | H | H | H |
| 41617 | 1 | H | H | m-D2 | H | H | A-22 | H | H | H | H |
| 41618 | 1 | H | H | m-D2 | H | H | A-23 | H | H | H | H |
| 41619 | 1 | H | H | m-D2 | H | H | A-24 | H | H | H | H |
| 41620 | 1 | H | H | m-D2 | H | H | A-25 | H | H | H | H |
| 41621 | 1 | H | H | m-D2 | H | H | A-26 | H | H | H | H |
| 41622 | 1 | H | H | m-D2 | H | H | A-27 | H | H | H | H |
| 41623 | 1 | H | H | m-D2 | H | H | A-28 | H | H | H | H |
| 41624 | 1 | H | H | m-D2 | H | H | A-29 | H | H | H | H |
| 41625 | 1 | H | H | m-D2 | H | H | A-30 | H | H | H | H |
| 41626 | 1 | H | H | m-D2 | H | H | A-31 | H | H | H | H |
| 41627 | 1 | H | H | m-D2 | H | H | A-32 | H | H | H | H |
| 41628 | 1 | H | H | m-D2 | H | H | A-33 | H | H | H | H |
| 41629 | 1 | H | H | m-D2 | H | H | A-34 | H | H | H | H |
| 41630 | 1 | H | H | m-D2 | H | H | A-35 | H | H | H | H |
| 41631 | 1 | H | H | m-D2 | H | H | A-36 | H | H | H | H |
| 41632 | 1 | H | H | m-D2 | H | H | A-37 | H | H | H | H |
| 41633 | 1 | H | H | m-D2 | H | H | A-38 | H | H | H | H |
| 41634 | 1 | H | H | m-D2 | H | H | A-39 | H | H | H | H |
| 41635 | 1 | H | H | m-D2 | H | H | A-40 | H | H | H | H |
| 41636 | 1 | H | H | m-D2 | H | H | A-41 | H | H | H | H |
| 41637 | 1 | H | H | m-D2 | H | H | A-42 | H | H | H | H |
| 41638 | 1 | H | H | m-D2 | H | H | A-43 | H | H | H | H |
| 41639 | 1 | H | H | m-D2 | H | H | A-44 | H | H | H | H |
| 41640 | 1 | H | H | m-D2 | H | H | A-45 | H | H | H | H |
| 41641 | 1 | H | H | m-D2 | H | H | A-46 | H | H | H | H |
| 41642 | 1 | H | H | m-D2 | H | H | A-47 | H | H | H | H |
| 41643 | 1 | H | H | m-D3 | H | H | A-1 | H | H | H | H |
| 41644 | 1 | H | H | m-D3 | H | H | A-2 | H | H | H | H |
| 41645 | 1 | H | H | m-D3 | H | H | A-3 | H | H | H | H |
| 41646 | 1 | H | H | m-D3 | H | H | A-4 | H | H | H | H |
| 41647 | 1 | H | H | m-D3 | H | H | A-5 | H | H | H | H |
| 41648 | 1 | H | H | m-D3 | H | H | A-6 | H | H | H | H |
| 41649 | 1 | H | H | m-D3 | H | H | A-7 | H | H | H | H |
| 41650 | 1 | H | H | m-D3 | H | H | A-8 | H | H | H | H |
| 41651 | 1 | H | H | m-D3 | H | H | A-9 | H | H | H | H |
| 41652 | 1 | H | H | m-D3 | H | H | A-10 | H | H | H | H |
| 41653 | 1 | H | H | m-D3 | H | H | A-11 | H | H | H | H |
| 41654 | 1 | H | H | m-D3 | H | H | A-12 | H | H | H | H |
| 41655 | 1 | H | H | m-D3 | H | H | A-13 | H | H | H | H |
| 41656 | 1 | H | H | m-D3 | H | H | A-14 | H | H | H | H |
| 41657 | 1 | H | H | m-D3 | H | H | A-15 | H | H | H | H |
| 41658 | 1 | H | H | m-D3 | H | H | A-16 | H | H | H | H |
| 41659 | 1 | H | H | m-D3 | H | H | A-17 | H | H | H | H |
| 41660 | 1 | H | H | m-D3 | H | H | A-18 | H | H | H | H |
| 41661 | 1 | H | H | m-D3 | H | H | A-19 | H | H | H | H |
| 41662 | 1 | H | H | m-D3 | H | H | A-20 | H | H | H | H |
| 41663 | 1 | H | H | m-D3 | H | H | A-21 | H | H | H | H |
| 41664 | 1 | H | H | m-D3 | H | H | A-22 | H | H | H | H |
| 41665 | 1 | H | H | m-D3 | H | H | A-23 | H | H | H | H |
| 41666 | 1 | H | H | m-D3 | H | H | A-24 | H | H | H | H |
| 41667 | 1 | H | H | m-D3 | H | H | A-25 | H | H | H | H |
| 41668 | 1 | H | H | m-D3 | H | H | A-26 | H | H | H | H |
| 41669 | 1 | H | H | m-D3 | H | H | A-27 | H | H | H | H |
| 41670 | 1 | H | H | m-D3 | H | H | A-28 | H | H | H | H |
| 41671 | 1 | H | H | m-D3 | H | H | A-29 | H | H | H | H |
| 41672 | 1 | H | H | m-D3 | H | H | A-30 | H | H | H | H |
| 41673 | 1 | H | H | m-D3 | H | H | A-31 | H | H | H | H |
| 41674 | 1 | H | H | m-D3 | H | H | A-32 | H | H | H | H |
| 41675 | 1 | H | H | m-D3 | H | H | A-33 | H | H | H | H |
| 41676 | 1 | H | H | m-D3 | H | H | A-34 | H | H | H | H |
| 41677 | 1 | H | H | m-D3 | H | H | A-35 | H | H | H | H |
| 41678 | 1 | H | H | m-D3 | H | H | A-36 | H | H | H | H |
| 41679 | 1 | H | H | m-D3 | H | H | A-37 | H | H | H | H |
| 41680 | 1 | H | H | m-D3 | H | H | A-38 | H | H | H | H |
| 41681 | 1 | H | H | m-D3 | H | H | A-39 | H | H | H | H |
| 41682 | 1 | H | H | m-D3 | H | H | A-40 | H | H | H | H |
| 41683 | 1 | H | H | m-D3 | H | H | A-41 | H | H | H | H |
| 41684 | 1 | H | H | m-D3 | H | H | A-42 | H | H | H | H |
| 41685 | 1 | H | H | m-D3 | H | H | A-43 | H | H | H | H |
| 41686 | 1 | H | H | m-D3 | H | H | A-44 | H | H | H | H |
| 41687 | 1 | H | H | m-D3 | H | H | A-45 | H | H | H | H |
| 41688 | 1 | H | H | m-D3 | H | H | A-46 | H | H | H | H |
| 41689 | 1 | H | H | m-D3 | H | H | A-47 | H | H | H | H |
| 41690 | 1 | H | H | m-D4 | H | H | A-1 | H | H | H | H |
| 41691 | 1 | H | H | m-D4 | H | H | A-2 | H | H | H | H |
| 41692 | 1 | H | H | m-D4 | H | H | A-3 | H | H | H | H |
| 41693 | 1 | H | H | m-D4 | H | H | A-4 | H | H | H | H |
| 41694 | 1 | H | H | m-D4 | H | H | A-5 | H | H | H | H |
| 41695 | 1 | H | H | m-D4 | H | H | A-6 | H | H | H | H |
| 41696 | 1 | H | H | m-D4 | H | H | A-7 | H | H | H | H |
| 41697 | 1 | H | H | m-D4 | H | H | A-8 | H | H | H | H |
| 41698 | 1 | H | H | m-D4 | H | H | A-9 | H | H | H | H |
| 41699 | 1 | H | H | m-D4 | H | H | A-10 | H | H | H | H |
| 41700 | 1 | H | H | m-D4 | H | H | A-11 | H | H | H | H |
| 41701 | 1 | H | H | m-D4 | H | H | A-12 | H | H | H | H |
| 41702 | 1 | H | H | m-D4 | H | H | A-13 | H | H | H | H |
| 41703 | 1 | H | H | m-D4 | H | H | A-14 | H | H | H | H |
| 41704 | 1 | H | H | m-D4 | H | H | A-15 | H | H | H | H |
| 41705 | 1 | H | H | m-D4 | H | H | A-16 | H | H | H | H |
| 41706 | 1 | H | H | m-D4 | H | H | A-17 | H | H | H | H |
| 41707 | 1 | H | H | m-D4 | H | H | A-18 | H | H | H | H |
| 41708 | 1 | H | H | m-D4 | H | H | A-19 | H | H | H | H |
| 41709 | 1 | H | H | m-D4 | H | H | A-20 | H | H | H | H |
| 41710 | 1 | H | H | m-D4 | H | H | A-21 | H | H | H | H |
| 41711 | 1 | H | H | m-D4 | H | H | A-22 | H | H | H | H |
| 41712 | 1 | H | H | m-D4 | H | H | A-23 | H | H | H | H |
| 41713 | 1 | H | H | m-D4 | H | H | A-24 | H | H | H | H |
| 41714 | 1 | H | H | m-D4 | H | H | A-25 | H | H | H | H |
| 41715 | 1 | H | H | m-D4 | H | H | A-26 | H | H | H | H |
| 41716 | 1 | H | H | m-D4 | H | H | A-27 | H | H | H | H |
| 41717 | 1 | H | H | m-D4 | H | H | A-28 | H | H | H | H |
| 41718 | 1 | H | H | m-D4 | H | H | A-29 | H | H | H | H |
| 41719 | 1 | H | H | m-D4 | H | H | A-30 | H | H | H | H |
| 41720 | 1 | H | H | m-D4 | H | H | A-31 | H | H | H | H |
| 41721 | 1 | H | H | m-D4 | H | H | A-32 | H | H | H | H |
| 41722 | 1 | H | H | m-D4 | H | H | A-33 | H | H | H | H |
| 41723 | 1 | H | H | m-D4 | H | H | A-34 | H | H | H | H |
| 41724 | 1 | H | H | m-D4 | H | H | A-35 | H | H | H | H |
| 41725 | 1 | H | H | m-D4 | H | H | A-36 | H | H | H | H |
| 41726 | 1 | H | H | m-D4 | H | H | A-37 | H | H | H | H |
| 41727 | 1 | H | H | m-D4 | H | H | A-38 | H | H | H | H |
| 41728 | 1 | H | H | m-D4 | H | H | A-39 | H | H | H | H |
| 41729 | 1 | H | H | m-D4 | H | H | A-40 | H | H | H | H |
| 41730 | 1 | H | H | m-D4 | H | H | A-41 | H | H | H | H |
| 41731 | 1 | H | H | m-D4 | H | H | A-42 | H | H | H | H |
| 41732 | 1 | H | H | m-D4 | H | H | A-43 | H | H | H | H |
| 41733 | 1 | H | H | m-D4 | H | H | A-44 | H | H | H | H |
| 41734 | 1 | H | H | m-D4 | H | H | A-45 | H | H | H | H |
| 41735 | 1 | H | H | m-D4 | H | H | A-46 | H | H | H | H |
| 41736 | 1 | H | H | m-D4 | H | H | A-47 | H | H | H | H |
| 41737 | 1 | H | H | m-D5 | H | H | A-1 | H | H | H | H |
| 41738 | 1 | H | H | m-D5 | H | H | A-2 | H | H | H | H |
| 41739 | 1 | H | H | m-D5 | H | H | A-3 | H | H | H | H |
| 41740 | 1 | H | H | m-D5 | H | H | A-4 | H | H | H | H |
| 41741 | 1 | H | H | m-D5 | H | H | A-5 | H | H | H | H |
| 41742 | 1 | H | H | m-D5 | H | H | A-6 | H | H | H | H |
| 41743 | 1 | H | H | m-D5 | H | H | A-7 | H | H | H | H |
| 41744 | 1 | H | H | m-D5 | H | H | A-8 | H | H | H | H |
| 41745 | 1 | H | H | m-D5 | H | H | A-9 | H | H | H | H |
| 41746 | 1 | H | H | m-D5 | H | H | A-10 | H | H | H | H |
| 41747 | 1 | H | H | m-D5 | H | H | A-11 | H | H | H | H |
| 41748 | 1 | H | H | m-D5 | H | H | A-12 | H | H | H | H |
| 41749 | 1 | H | H | m-D5 | H | H | A-13 | H | H | H | H |
| 41750 | 1 | H | H | m-D5 | H | H | A-14 | H | H | H | H |
| 41751 | 1 | H | H | m-D5 | H | H | A-15 | H | H | H | H |
| 41752 | 1 | H | H | m-D5 | H | H | A-16 | H | H | H | H |
| 41753 | 1 | H | H | m-D5 | H | H | A-17 | H | H | H | H |
| 41754 | 1 | H | H | m-D5 | H | H | A-18 | H | H | H | H |
| 41755 | 1 | H | H | m-D5 | H | H | A-19 | H | H | H | H |
| 41756 | 1 | H | H | m-D5 | H | H | A-20 | H | H | H | H |
| 41757 | 1 | H | H | m-D5 | H | H | A-21 | H | H | H | H |
| 41758 | 1 | H | H | m-D5 | H | H | A-22 | H | H | H | H |
| 41759 | 1 | H | H | m-D5 | H | H | A-23 | H | H | H | H |
| 41760 | 1 | H | H | m-D5 | H | H | A-24 | H | H | H | H |
| 41761 | 1 | H | H | m-D5 | H | H | A-25 | H | H | H | H |
| 41762 | 1 | H | H | m-D5 | H | H | A-26 | H | H | H | H |
| 41763 | 1 | H | H | m-D5 | H | H | A-27 | H | H | H | H |
| 41764 | 1 | H | H | m-D5 | H | H | A-28 | H | H | H | H |
| 41765 | 1 | H | H | m-D5 | H | H | A-29 | H | H | H | H |
| 41766 | 1 | H | H | m-D5 | H | H | A-30 | H | H | H | H |
| 41767 | 1 | H | H | m-D5 | H | H | A-31 | H | H | H | H |
| 41768 | 1 | H | H | m-D5 | H | H | A-32 | H | H | H | H |
| 41769 | 1 | H | H | m-D5 | H | H | A-33 | H | H | H | H |
| 41770 | 1 | H | H | m-D5 | H | H | A-34 | H | H | H | H |
| 41771 | 1 | H | H | m-D5 | H | H | A-35 | H | H | H | H |
| 41772 | 1 | H | H | m-D5 | H | H | A-36 | H | H | H | H |
| 41773 | 1 | H | H | m-D5 | H | H | A-37 | H | H | H | H |
| 41774 | 1 | H | H | m-D5 | H | H | A-38 | H | H | H | H |
| 41775 | 1 | H | H | m-D5 | H | H | A-39 | H | H | H | H |
| 41776 | 1 | H | H | m-D5 | H | H | A-40 | H | H | H | H |
| 41777 | 1 | H | H | m-D5 | H | H | A-41 | H | H | H | H |
| 41778 | 1 | H | H | m-D5 | H | H | A-42 | H | H | H | H |
| 41779 | 1 | H | H | m-D5 | H | H | A-43 | H | H | H | H |
| 41780 | 1 | H | H | m-D5 | H | H | A-44 | H | H | H | H |
| 41781 | 1 | H | H | m-D5 | H | H | A-45 | H | H | H | H |
| 41782 | 1 | H | H | m-D5 | H | H | A-46 | H | H | H | H |
| 41783 | 1 | H | H | m-D5 | H | H | A-47 | H | H | H | H |
| 41784 | 1 | H | H | m-D6 | H | H | A-1 | H | H | H | H |
| 41785 | 1 | H | H | m-D6 | H | H | A-2 | H | H | H | H |
| 41786 | 1 | H | H | m-D6 | H | H | A-3 | H | H | H | H |
| 41787 | 1 | H | H | m-D6 | H | H | A-4 | H | H | H | H |
| 41788 | 1 | H | H | m-D6 | H | H | A-5 | H | H | H | H |
| 41789 | 1 | H | H | m-D6 | H | H | A-6 | H | H | H | H |
| 41790 | 1 | H | H | m-D6 | H | H | A-7 | H | H | H | H |
| 41791 | 1 | H | H | m-D6 | H | H | A-8 | H | H | H | H |
| 41792 | 1 | H | H | m-D6 | H | H | A-9 | H | H | H | H |
| 41793 | 1 | H | H | m-D6 | H | H | A-10 | H | H | H | H |
| 41794 | 1 | H | H | m-D6 | H | H | A-11 | H | H | H | H |
| 41795 | 1 | H | H | m-D6 | H | H | A-12 | H | H | H | H |
| 41796 | 1 | H | H | m-D6 | H | H | A-13 | H | H | H | H |
| 41797 | 1 | H | H | m-D6 | H | H | A-14 | H | H | H | H |
| 41798 | 1 | H | H | m-D6 | H | H | A-15 | H | H | H | H |
| 41799 | 1 | H | H | m-D6 | H | H | A-16 | H | H | H | H |
| 41800 | 1 | H | H | m-D6 | H | H | A-17 | H | H | H | H |
| 41801 | 1 | H | H | m-D6 | H | H | A-18 | H | H | H | H |
| 41802 | 1 | H | H | m-D6 | H | H | A-19 | H | H | H | H |
| 41803 | 1 | H | H | m-D6 | H | H | A-20 | H | H | H | H |
| 41804 | 1 | H | H | m-D6 | H | H | A-21 | H | H | H | H |
| 41805 | 1 | H | H | m-D6 | H | H | A-22 | H | H | H | H |
| 41806 | 1 | H | H | m-D6 | H | H | A-23 | H | H | H | H |
| 41807 | 1 | H | H | m-D6 | H | H | A-24 | H | H | H | H |
| 41808 | 1 | H | H | m-D6 | H | H | A-25 | H | H | H | H |
| 41809 | 1 | H | H | m-D6 | H | H | A-26 | H | H | H | H |
| 41810 | 1 | H | H | m-D6 | H | H | A-27 | H | H | H | H |
| 41811 | 1 | H | H | m-D6 | H | H | A-28 | H | H | H | H |
| 41812 | 1 | H | H | m-D6 | H | H | A-29 | H | H | H | H |
| 41813 | 1 | H | H | m-D6 | H | H | A-30 | H | H | H | H |
| 41814 | 1 | H | H | m-D6 | H | H | A-31 | H | H | H | H |
| 41815 | 1 | H | H | m-D6 | H | H | A-32 | H | H | H | H |
| 41816 | 1 | H | H | m-D6 | H | H | A-33 | H | H | H | H |
| 41817 | 1 | H | H | m-D6 | H | H | A-34 | H | H | H | H |
| 41818 | 1 | H | H | m-D6 | H | H | A-35 | H | H | H | H |
| 41819 | 1 | H | H | m-D6 | H | H | A-36 | H | H | H | H |
| 41820 | 1 | H | H | m-D6 | H | H | A-37 | H | H | H | H |
| 41821 | 1 | H | H | m-D6 | H | H | A-38 | H | H | H | H |
| 41822 | 1 | H | H | m-D6 | H | H | A-39 | H | H | H | H |
| 41823 | 1 | H | H | m-D6 | H | H | A-40 | H | H | H | H |
| 41824 | 1 | H | H | m-D6 | H | H | A-41 | H | H | H | H |
| 41825 | 1 | H | H | m-D6 | H | H | A-42 | H | H | H | H |
| 41826 | 1 | H | H | m-D6 | H | H | A-43 | H | H | H | H |
| 41827 | 1 | H | H | m-D6 | H | H | A-44 | H | H | H | H |
| 41828 | 1 | H | H | m-D6 | H | H | A-45 | H | H | H | H |
| 41829 | 1 | H | H | m-D6 | H | H | A-46 | H | H | H | H |
| 41830 | 1 | H | H | m-D6 | H | H | A-47 | H | H | H | H |
| 41831 | 1 | H | H | m-D7 | H | H | A-1 | H | H | H | H |
| 41832 | 1 | H | H | m-D7 | H | H | A-2 | H | H | H | H |
| 41833 | 1 | H | H | m-D7 | H | H | A-3 | H | H | H | H |
| 41834 | 1 | H | H | m-D7 | H | H | A-4 | H | H | H | H |
| 41835 | 1 | H | H | m-D7 | H | H | A-5 | H | H | H | H |
| 41836 | 1 | H | H | m-D7 | H | H | A-6 | H | H | H | H |
| 41837 | 1 | H | H | m-D7 | H | H | A-7 | H | H | H | H |
| 41838 | 1 | H | H | m-D7 | H | H | A-8 | H | H | H | H |
| 41839 | 1 | H | H | m-D7 | H | H | A-9 | H | H | H | H |
| 41840 | 1 | H | H | m-D7 | H | H | A-10 | H | H | H | H |
| 41841 | 1 | H | H | m-D7 | H | H | A-11 | H | H | H | H |
| 41842 | 1 | H | H | m-D7 | H | H | A-12 | H | H | H | H |
| 41843 | 1 | H | H | m-D7 | H | H | A-13 | H | H | H | H |
| 41844 | 1 | H | H | m-D7 | H | H | A-14 | H | H | H | H |
| 41845 | 1 | H | H | m-D7 | H | H | A-15 | H | H | H | H |
| 41846 | 1 | H | H | m-D7 | H | H | A-16 | H | H | H | H |
| 41847 | 1 | H | H | m-D7 | H | H | A-17 | H | H | H | H |
| 41848 | 1 | H | H | m-D7 | H | H | A-18 | H | H | H | H |
| 41849 | 1 | H | H | m-D7 | H | H | A-19 | H | H | H | H |
| 41850 | 1 | H | H | m-D7 | H | H | A-20 | H | H | H | H |
| 41851 | 1 | H | H | m-D7 | H | H | A-21 | H | H | H | H |
| 41852 | 1 | H | H | m-D7 | H | H | A-22 | H | H | H | H |
| 41853 | 1 | H | H | m-D7 | H | H | A-23 | H | H | H | H |
| 41854 | 1 | H | H | m-D7 | H | H | A-24 | H | H | H | H |
| 41855 | 1 | H | H | m-D7 | H | H | A-25 | H | H | H | H |
| 41856 | 1 | H | H | m-D7 | H | H | A-26 | H | H | H | H |
| 41857 | 1 | H | H | m-D7 | H | H | A-27 | H | H | H | H |
| 41858 | 1 | H | H | m-D7 | H | H | A-28 | H | H | H | H |
| 41859 | 1 | H | H | m-D7 | H | H | A-29 | H | H | H | H |
| 41860 | 1 | H | H | m-D7 | H | H | A-30 | H | H | H | H |
| 41861 | 1 | H | H | m-D7 | H | H | A-31 | H | H | H | H |
| 41862 | 1 | H | H | m-D7 | H | H | A-32 | H | H | H | H |
| 41863 | 1 | H | H | m-D7 | H | H | A-33 | H | H | H | H |
| 41864 | 1 | H | H | m-D7 | H | H | A-34 | H | H | H | H |
| 41865 | 1 | H | H | m-D7 | H | H | A-35 | H | H | H | H |
| 41866 | 1 | H | H | m-D7 | H | H | A-36 | H | H | H | H |
| 41867 | 1 | H | H | m-D7 | H | H | A-37 | H | H | H | H |
| 41868 | 1 | H | H | m-D7 | H | H | A-38 | H | H | H | H |
| 41869 | 1 | H | H | m-D7 | H | H | A-39 | H | H | H | H |
| 41870 | 1 | H | H | m-D7 | H | H | A-40 | H | H | H | H |
| 41871 | 1 | H | H | m-D7 | H | H | A-41 | H | H | H | H |
| 41872 | 1 | H | H | m-D7 | H | H | A-42 | H | H | H | H |
| 41873 | 1 | H | H | m-D7 | H | H | A-43 | H | H | H | H |
| 41874 | 1 | H | H | m-D7 | H | H | A-44 | H | H | H | H |
| 41875 | 1 | H | H | m-D7 | H | H | A-45 | H | H | H | H |
| 41876 | 1 | H | H | m-D7 | H | H | A-46 | H | H | H | H |
| 41877 | 1 | H | H | m-D7 | H | H | A-47 | H | H | H | H |
| 41878 | 1 | H | H | m-D8 | H | H | A-1 | H | H | H | H |
| 41879 | 1 | H | H | m-D8 | H | H | A-2 | H | H | H | H |
| 41880 | 1 | H | H | m-D8 | H | H | A-3 | H | H | H | H |
| 41881 | 1 | H | H | m-D8 | H | H | A-4 | H | H | H | H |
| 41882 | 1 | H | H | m-D8 | H | H | A-5 | H | H | H | H |
| 41883 | 1 | H | H | m-D8 | H | H | A-6 | H | H | H | H |
| 41884 | 1 | H | H | m-D8 | H | H | A-7 | H | H | H | H |
| 41885 | 1 | H | H | m-D8 | H | H | A-8 | H | H | H | H |
| 41886 | 1 | H | H | m-D8 | H | H | A-9 | H | H | H | H |
| 41887 | 1 | H | H | m-D8 | H | H | A-10 | H | H | H | H |
| 41888 | 1 | H | H | m-D8 | H | H | A-11 | H | H | H | H |
| 41889 | 1 | H | H | m-D8 | H | H | A-12 | H | H | H | H |
| 41890 | 1 | H | H | m-D8 | H | H | A-13 | H | H | H | H |
| 41891 | 1 | H | H | m-D8 | H | H | A-14 | H | H | H | H |
| 41892 | 1 | H | H | m-D8 | H | H | A-15 | H | H | H | H |
| 41893 | 1 | H | H | m-D8 | H | H | A-16 | H | H | H | H |
| 41894 | 1 | H | H | m-D8 | H | H | A-17 | H | H | H | H |
| 41895 | 1 | H | H | m-D8 | H | H | A-18 | H | H | H | H |
| 41896 | 1 | H | H | m-D8 | H | H | A-19 | H | H | H | H |
| 41897 | 1 | H | H | m-D8 | H | H | A-20 | H | H | H | H |
| 41898 | 1 | H | H | m-D8 | H | H | A-21 | H | H | H | H |
| 41899 | 1 | H | H | m-D8 | H | H | A-22 | H | H | H | H |
| 41900 | 1 | H | H | m-D8 | H | H | A-23 | H | H | H | H |
| 41901 | 1 | H | H | m-D8 | H | H | A-24 | H | H | H | H |
| 41902 | 1 | H | H | m-D8 | H | H | A-25 | H | H | H | H |
| 41903 | 1 | H | H | m-D8 | H | H | A-26 | H | H | H | H |
| 41904 | 1 | H | H | m-D8 | H | H | A-27 | H | H | H | H |
| 41905 | 1 | H | H | m-D8 | H | H | A-28 | H | H | H | H |
| 41906 | 1 | H | H | m-D8 | H | H | A-29 | H | H | H | H |
| 41907 | 1 | H | H | m-D8 | H | H | A-30 | H | H | H | H |
| 41908 | 1 | H | H | m-D8 | H | H | A-31 | H | H | H | H |
| 41909 | 1 | H | H | m-D8 | H | H | A-32 | H | H | H | H |
| 41910 | 1 | H | H | m-D8 | H | H | A-33 | H | H | H | H |
| 41911 | 1 | H | H | m-D8 | H | H | A-34 | H | H | H | H |
| 41912 | 1 | H | H | m-D8 | H | H | A-35 | H | H | H | H |
| 41913 | 1 | H | H | m-D8 | H | H | A-36 | H | H | H | H |
| 41914 | 1 | H | H | m-D8 | H | H | A-37 | H | H | H | H |
| 41915 | 1 | H | H | m-D8 | H | H | A-38 | H | H | H | H |
| 41916 | 1 | H | H | m-D8 | H | H | A-39 | H | H | H | H |
| 41917 | 1 | H | H | m-D8 | H | H | A-40 | H | H | H | H |
| 41918 | 1 | H | H | m-D8 | H | H | A-41 | H | H | H | H |
| 41919 | 1 | H | H | m-D8 | H | H | A-42 | H | H | H | H |
| 41920 | 1 | H | H | m-D8 | H | H | A-43 | H | H | H | H |
| 41921 | 1 | H | H | m-D8 | H | H | A-44 | H | H | H | H |
| 41922 | 1 | H | H | m-D8 | H | H | A-45 | H | H | H | H |
| 41923 | 1 | H | H | m-D8 | H | H | A-46 | H | H | H | H |
| 41924 | 1 | H | H | m-D8 | H | H | A-47 | H | H | H | H |
| 41925 | 1 | H | H | m-D9 | H | H | A-1 | H | H | H | H |
| 41926 | 1 | H | H | m-D9 | H | H | A-2 | H | H | H | H |
| 41927 | 1 | H | H | m-D9 | H | H | A-3 | H | H | H | H |
| 41928 | 1 | H | H | m-D9 | H | H | A-4 | H | H | H | H |
| 41929 | 1 | H | H | m-D9 | H | H | A-5 | H | H | H | H |
| 41930 | 1 | H | H | m-D9 | H | H | A-6 | H | H | H | H |
| 41931 | 1 | H | H | m-D9 | H | H | A-7 | H | H | H | H |
| 41932 | 1 | H | H | m-D9 | H | H | A-8 | H | H | H | H |
| 41933 | 1 | H | H | m-D9 | H | H | A-9 | H | H | H | H |
| 41934 | 1 | H | H | m-D9 | H | H | A-10 | H | H | H | H |
| 41935 | 1 | H | H | m-D9 | H | H | A-11 | H | H | H | H |
| 41936 | 1 | H | H | m-D9 | H | H | A-12 | H | H | H | H |
| 41937 | 1 | H | H | m-D9 | H | H | A-13 | H | H | H | H |
| 41938 | 1 | H | H | m-D9 | H | H | A-14 | H | H | H | H |
| 41939 | 1 | H | H | m-D9 | H | H | A-15 | H | H | H | H |
| 41940 | 1 | H | H | m-D9 | H | H | A-16 | H | H | H | H |
| 41941 | 1 | H | H | m-D9 | H | H | A-17 | H | H | H | H |
| 41942 | 1 | H | H | m-D9 | H | H | A-18 | H | H | H | H |
| 41943 | 1 | H | H | m-D9 | H | H | A-19 | H | H | H | H |
| 41944 | 1 | H | H | m-D9 | H | H | A-20 | H | H | H | H |
| 41945 | 1 | H | H | m-D9 | H | H | A-21 | H | H | H | H |
| 41946 | 1 | H | H | m-D9 | H | H | A-22 | H | H | H | H |
| 41947 | 1 | H | H | m-D9 | H | H | A-23 | H | H | H | H |
| 41948 | 1 | H | H | m-D9 | H | H | A-24 | H | H | H | H |
| 41949 | 1 | H | H | m-D9 | H | H | A-25 | H | H | H | H |
| 41950 | 1 | H | H | m-D9 | H | H | A-26 | H | H | H | H |
| 41951 | 1 | H | H | m-D9 | H | H | A-27 | H | H | H | H |
| 41952 | 1 | H | H | m-D9 | H | H | A-28 | H | H | H | H |
| 41953 | 1 | H | H | m-D9 | H | H | A-29 | H | H | H | H |
| 41954 | 1 | H | H | m-D9 | H | H | A-30 | H | H | H | H |
| 41955 | 1 | H | H | m-D9 | H | H | A-31 | H | H | H | H |
| 41956 | 1 | H | H | m-D9 | H | H | A-32 | H | H | H | H |
| 41957 | 1 | H | H | m-D9 | H | H | A-33 | H | H | H | H |
| 41958 | 1 | H | H | m-D9 | H | H | A-34 | H | H | H | H |
| 41959 | 1 | H | H | m-D9 | H | H | A-35 | H | H | H | H |
| 41960 | 1 | H | H | m-D9 | H | H | A-36 | H | H | H | H |
| 41961 | 1 | H | H | m-D9 | H | H | A-37 | H | H | H | H |
| 41962 | 1 | H | H | m-D9 | H | H | A-38 | H | H | H | H |
| 41963 | 1 | H | H | m-D9 | H | H | A-39 | H | H | H | H |
| 41964 | 1 | H | H | m-D9 | H | H | A-40 | H | H | H | H |
| 41965 | 1 | H | H | m-D9 | H | H | A-41 | H | H | H | H |
| 41966 | 1 | H | H | m-D9 | H | H | A-42 | H | H | H | H |
| 41967 | 1 | H | H | m-D9 | H | H | A-43 | H | H | H | H |
| 41968 | 1 | H | H | m-D9 | H | H | A-44 | H | H | H | H |
| 41969 | 1 | H | H | m-D9 | H | H | A-45 | H | H | H | H |
| 41970 | 1 | H | H | m-D9 | H | H | A-46 | H | H | H | H |
| 41971 | 1 | H | H | m-D9 | H | H | A-47 | H | H | H | H |
| 41972 | 1 | H | H | m-D10 | H | H | A-1 | H | H | H | H |
| 41973 | 1 | H | H | m-D10 | H | H | A-2 | H | H | H | H |
| 41974 | 1 | H | H | m-D10 | H | H | A-3 | H | H | H | H |
| 41975 | 1 | H | H | m-D10 | H | H | A-4 | H | H | H | H |
| 41976 | 1 | H | H | m-D10 | H | H | A-5 | H | H | H | H |
| 41977 | 1 | H | H | m-D10 | H | H | A-6 | H | H | H | H |
| 41978 | 1 | H | H | m-D10 | H | H | A-7 | H | H | H | H |
| 41979 | 1 | H | H | m-D10 | H | H | A-8 | H | H | H | H |
| 41980 | 1 | H | H | m-D10 | H | H | A-9 | H | H | H | H |
| 41981 | 1 | H | H | m-D10 | H | H | A-10 | H | H | H | H |
| 41982 | 1 | H | H | m-D10 | H | H | A-11 | H | H | H | H |
| 41983 | 1 | H | H | m-D10 | H | H | A-12 | H | H | H | H |
| 41984 | 1 | H | H | m-D10 | H | H | A-13 | H | H | H | H |
| 41985 | 1 | H | H | m-D10 | H | H | A-14 | H | H | H | H |
| 41986 | 1 | H | H | m-D10 | H | H | A-15 | H | H | H | H |
| 41987 | 1 | H | H | m-D10 | H | H | A-16 | H | H | H | H |
| 41988 | 1 | H | H | m-D10 | H | H | A-17 | H | H | H | H |
| 41989 | 1 | H | H | m-D10 | H | H | A-18 | H | H | H | H |
| 41990 | 1 | H | H | m-D10 | H | H | A-19 | H | H | H | H |
| 41991 | 1 | H | H | m-D10 | H | H | A-20 | H | H | H | H |
| 41992 | 1 | H | H | m-D10 | H | H | A-21 | H | H | H | H |
| 41993 | 1 | H | H | m-D10 | H | H | A-22 | H | H | H | H |
| 41994 | 1 | H | H | m-D10 | H | H | A-23 | H | H | H | H |
| 41995 | 1 | H | H | m-D10 | H | H | A-24 | H | H | H | H |
| 41996 | 1 | H | H | m-D10 | H | H | A-25 | H | H | H | H |
| 41997 | 1 | H | H | m-D10 | H | H | A-26 | H | H | H | H |
| 41998 | 1 | H | H | m-D10 | H | H | A-27 | H | H | H | H |
| 41999 | 1 | H | H | m-D10 | H | H | A-28 | H | H | H | H |
| 42000 | 1 | H | H | m-D10 | H | H | A-29 | H | H | H | H |
| 42001 | 1 | H | H | m-D10 | H | H | A-30 | H | H | H | H |
| 42002 | 1 | H | H | m-D10 | H | H | A-31 | H | H | H | H |
| 42003 | 1 | H | H | m-D10 | H | H | A-32 | H | H | H | H |
| 42004 | 1 | H | H | m-D10 | H | H | A-33 | H | H | H | H |
| 42005 | 1 | H | H | m-D10 | H | H | A-34 | H | H | H | H |
| 42006 | 1 | H | H | m-D10 | H | H | A-35 | H | H | H | H |
| 42007 | 1 | H | H | m-D10 | H | H | A-36 | H | H | H | H |
| 42008 | 1 | H | H | m-D10 | H | H | A-37 | H | H | H | H |
| 42009 | 1 | H | H | m-D10 | H | H | A-38 | H | H | H | H |
| 42010 | 1 | H | H | m-D10 | H | H | A-39 | H | H | H | H |
| 42011 | 1 | H | H | m-D10 | H | H | A-40 | H | H | H | H |
| 42012 | 1 | H | H | m-D10 | H | H | A-41 | H | H | H | H |
| 42013 | 1 | H | H | m-D10 | H | H | A-42 | H | H | H | H |
| 42014 | 1 | H | H | m-D10 | H | H | A-43 | H | H | H | H |
| 42015 | 1 | H | H | m-D10 | H | H | A-44 | H | H | H | H |
| 42016 | 1 | H | H | m-D10 | H | H | A-45 | H | H | H | H |
| 42017 | 1 | H | H | m-D10 | H | H | A-46 | H | H | H | H |
| 42018 | 1 | H | H | m-D10 | H | H | A-47 | H | H | H | H |
| 42019 | 1 | H | H | m-D11 | H | H | A-1 | H | H | H | H |
| 42020 | 1 | H | H | m-D11 | H | H | A-2 | H | H | H | H |
| 42021 | 1 | H | H | m-D11 | H | H | A-3 | H | H | H | H |
| 42022 | 1 | H | H | m-D11 | H | H | A-4 | H | H | H | H |
| 42023 | 1 | H | H | m-D11 | H | H | A-5 | H | H | H | H |
| 42024 | 1 | H | H | m-D11 | H | H | A-6 | H | H | H | H |
| 42025 | 1 | H | H | m-D11 | H | H | A-7 | H | H | H | H |
| 42026 | 1 | H | H | m-D11 | H | H | A-8 | H | H | H | H |
| 42027 | 1 | H | H | m-D11 | H | H | A-9 | H | H | H | H |
| 42028 | 1 | H | H | m-D11 | H | H | A-10 | H | H | H | H |
| 42029 | 1 | H | H | m-D11 | H | H | A-11 | H | H | H | H |
| 42030 | 1 | H | H | m-D11 | H | H | A-12 | H | H | H | H |
| 42031 | 1 | H | H | m-D11 | H | H | A-13 | H | H | H | H |
| 42032 | 1 | H | H | m-D11 | H | H | A-14 | H | H | H | H |
| 42033 | 1 | H | H | m-D11 | H | H | A-15 | H | H | H | H |
| 42034 | 1 | H | H | m-D11 | H | H | A-16 | H | H | H | H |
| 42035 | 1 | H | H | m-D11 | H | H | A-17 | H | H | H | H |
| 42036 | 1 | H | H | m-D11 | H | H | A-18 | H | H | H | H |
| 42037 | 1 | H | H | m-D11 | H | H | A-19 | H | H | H | H |
| 42038 | 1 | H | H | m-D11 | H | H | A-20 | H | H | H | H |
| 42039 | 1 | H | H | m-D11 | H | H | A-21 | H | H | H | H |
| 42040 | 1 | H | H | m-D11 | H | H | A-22 | H | H | H | H |
| 42041 | 1 | H | H | m-D11 | H | H | A-23 | H | H | H | H |
| 42042 | 1 | H | H | m-D11 | H | H | A-24 | H | H | H | H |
| 42043 | 1 | H | H | m-D11 | H | H | A-25 | H | H | H | H |
| 42044 | 1 | H | H | m-D11 | H | H | A-26 | H | H | H | H |
| 42045 | 1 | H | H | m-D11 | H | H | A-27 | H | H | H | H |
| 42046 | 1 | H | H | m-D11 | H | H | A-28 | H | H | H | H |
| 42047 | 1 | H | H | m-D11 | H | H | A-29 | H | H | H | H |
| 42048 | 1 | H | H | m-D11 | H | H | A-30 | H | H | H | H |
| 42049 | 1 | H | H | m-D11 | H | H | A-31 | H | H | H | H |
| 42050 | 1 | H | H | m-D11 | H | H | A-32 | H | H | H | H |
| 42051 | 1 | H | H | m-D11 | H | H | A-33 | H | H | H | H |
| 42052 | 1 | H | H | m-D11 | H | H | A-34 | H | H | H | H |
| 42053 | 1 | H | H | m-D11 | H | H | A-35 | H | H | H | H |
| 42054 | 1 | H | H | m-D11 | H | H | A-36 | H | H | H | H |
| 42055 | 1 | H | H | m-D11 | H | H | A-37 | H | H | H | H |
| 42056 | 1 | H | H | m-D11 | H | H | A-38 | H | H | H | H |
| 42057 | 1 | H | H | m-D11 | H | H | A-39 | H | H | H | H |
| 42058 | 1 | H | H | m-D11 | H | H | A-40 | H | H | H | H |
| 42059 | 1 | H | H | m-D11 | H | H | A-41 | H | H | H | H |
| 42060 | 1 | H | H | m-D11 | H | H | A-42 | H | H | H | H |
| 42061 | 1 | H | H | m-D11 | H | H | A-43 | H | H | H | H |
| 42062 | 1 | H | H | m-D11 | H | H | A-44 | H | H | H | H |
| 42063 | 1 | H | H | m-D11 | H | H | A-45 | H | H | H | H |
| 42064 | 1 | H | H | m-D11 | H | H | A-46 | H | H | H | H |
| 42065 | 1 | H | H | m-D11 | H | H | A-47 | H | H | H | H |
| 42066 | 1 | H | H | m-D12 | H | H | A-1 | H | H | H | H |
| 42067 | 1 | H | H | m-D12 | H | H | A-2 | H | H | H | H |
| 42068 | 1 | H | H | m-D12 | H | H | A-3 | H | H | H | H |
| 42069 | 1 | H | H | m-D12 | H | H | A-4 | H | H | H | H |
| 42070 | 1 | H | H | m-D12 | H | H | A-5 | H | H | H | H |
| 42071 | 1 | H | H | m-D12 | H | H | A-6 | H | H | H | H |
| 42072 | 1 | H | H | m-D12 | H | H | A-7 | H | H | H | H |
| 42073 | 1 | H | H | m-D12 | H | H | A-8 | H | H | H | H |
| 42074 | 1 | H | H | m-D12 | H | H | A-9 | H | H | H | H |
| 42075 | 1 | H | H | m-D12 | H | H | A-10 | H | H | H | H |
| 42076 | 1 | H | H | m-D12 | H | H | A-11 | H | H | H | H |
| 42077 | 1 | H | H | m-D12 | H | H | A-12 | H | H | H | H |
| 42078 | 1 | H | H | m-D12 | H | H | A-13 | H | H | H | H |
| 42079 | 1 | H | H | m-D12 | H | H | A-14 | H | H | H | H |
| 42080 | 1 | H | H | m-D12 | H | H | A-15 | H | H | H | H |
| 42081 | 1 | H | H | m-D12 | H | H | A-16 | H | H | H | H |
| 42082 | 1 | H | H | m-D12 | H | H | A-17 | H | H | H | H |
| 42083 | 1 | H | H | m-D12 | H | H | A-18 | H | H | H | H |
| 42084 | 1 | H | H | m-D12 | H | H | A-19 | H | H | H | H |
| 42085 | 1 | H | H | m-D12 | H | H | A-20 | H | H | H | H |
| 42086 | 1 | H | H | m-D12 | H | H | A-21 | H | H | H | H |
| 42087 | 1 | H | H | m-D12 | H | H | A-22 | H | H | H | H |
| 42088 | 1 | H | H | m-D12 | H | H | A-23 | H | H | H | H |
| 42089 | 1 | H | H | m-D12 | H | H | A-24 | H | H | H | H |
| 42090 | 1 | H | H | m-D12 | H | H | A-25 | H | H | H | H |
| 42091 | 1 | H | H | m-D12 | H | H | A-26 | H | H | H | H |
| 42092 | 1 | H | H | m-D12 | H | H | A-27 | H | H | H | H |
| 42093 | 1 | H | H | m-D12 | H | H | A-28 | H | H | H | H |
| 42094 | 1 | H | H | m-D12 | H | H | A-29 | H | H | H | H |
| 42095 | 1 | H | H | m-D12 | H | H | A-30 | H | H | H | H |
| 42096 | 1 | H | H | m-D12 | H | H | A-31 | H | H | H | H |
| 42097 | 1 | H | H | m-D12 | H | H | A-32 | H | H | H | H |
| 42098 | 1 | H | H | m-D12 | H | H | A-33 | H | H | H | H |
| 42099 | 1 | H | H | m-D12 | H | H | A-34 | H | H | H | H |
| 42100 | 1 | H | H | m-D12 | H | H | A-35 | H | H | H | H |
| 42101 | 1 | H | H | m-D12 | H | H | A-36 | H | H | H | H |
| 42102 | 1 | H | H | m-D12 | H | H | A-37 | H | H | H | H |
| 42103 | 1 | H | H | m-D12 | H | H | A-38 | H | H | H | H |
| 42104 | 1 | H | H | m-D12 | H | H | A-39 | H | H | H | H |
| 42105 | 1 | H | H | m-D12 | H | H | A-40 | H | H | H | H |
| 42106 | 1 | H | H | m-D12 | H | H | A-41 | H | H | H | H |
| 42107 | 1 | H | H | m-D12 | H | H | A-42 | H | H | H | H |
| 42108 | 1 | H | H | m-D12 | H | H | A-43 | H | H | H | H |
| 42109 | 1 | H | H | m-D12 | H | H | A-44 | H | H | H | H |
| 42110 | 1 | H | H | m-D12 | H | H | A-45 | H | H | H | H |
| 42111 | 1 | H | H | m-D12 | H | H | A-46 | H | H | H | H |
| 42112 | 1 | H | H | m-D12 | H | H | A-47 | H | H | H | H |
| 42113 | 1 | H | H | m-D13 | H | H | A-1 | H | H | H | H |
| 42114 | 1 | H | H | m-D13 | H | H | A-2 | H | H | H | H |
| 42115 | 1 | H | H | m-D13 | H | H | A-3 | H | H | H | H |
| 42116 | 1 | H | H | m-D13 | H | H | A-4 | H | H | H | H |
| 42117 | 1 | H | H | m-D13 | H | H | A-5 | H | H | H | H |
| 42118 | 1 | H | H | m-D13 | H | H | A-6 | H | H | H | H |
| 42119 | 1 | H | H | m-D13 | H | H | A-7 | H | H | H | H |
| 42120 | 1 | H | H | m-D13 | H | H | A-8 | H | H | H | H |
| 42121 | 1 | H | H | m-D13 | H | H | A-9 | H | H | H | H |
| 42122 | 1 | H | H | m-D13 | H | H | A-10 | H | H | H | H |
| 42123 | 1 | H | H | m-D13 | H | H | A-11 | H | H | H | H |
| 42124 | 1 | H | H | m-D13 | H | H | A-12 | H | H | H | H |
| 42125 | 1 | H | H | m-D13 | H | H | A-13 | H | H | H | H |
| 42126 | 1 | H | H | m-D13 | H | H | A-14 | H | H | H | H |
| 42127 | 1 | H | H | m-D13 | H | H | A-15 | H | H | H | H |
| 42128 | 1 | H | H | m-D13 | H | H | A-16 | H | H | H | H |
| 42129 | 1 | H | H | m-D13 | H | H | A-17 | H | H | H | H |
| 42130 | 1 | H | H | m-D13 | H | H | A-18 | H | H | H | H |
| 42131 | 1 | H | H | m-D13 | H | H | A-19 | H | H | H | H |
| 42132 | 1 | H | H | m-D13 | H | H | A-20 | H | H | H | H |
| 42133 | 1 | H | H | m-D13 | H | H | A-21 | H | H | H | H |
| 42134 | 1 | H | H | m-D13 | H | H | A-22 | H | H | H | H |
| 42135 | 1 | H | H | m-D13 | H | H | A-23 | H | H | H | H |
| 42136 | 1 | H | H | m-D13 | H | H | A-24 | H | H | H | H |
| 42137 | 1 | H | H | m-D13 | H | H | A-25 | H | H | H | H |
| 42138 | 1 | H | H | m-D13 | H | H | A-26 | H | H | H | H |
| 42139 | 1 | H | H | m-D13 | H | H | A-27 | H | H | H | H |
| 42140 | 1 | H | H | m-D13 | H | H | A-28 | H | H | H | H |
| 42141 | 1 | H | H | m-D13 | H | H | A-29 | H | H | H | H |
| 42142 | 1 | H | H | m-D13 | H | H | A-30 | H | H | H | H |
| 42143 | 1 | H | H | m-D13 | H | H | A-31 | H | H | H | H |
| 42144 | 1 | H | H | m-D13 | H | H | A-32 | H | H | H | H |
| 42145 | 1 | H | H | m-D13 | H | H | A-33 | H | H | H | H |
| 42146 | 1 | H | H | m-D13 | H | H | A-34 | H | H | H | H |
| 42147 | 1 | H | H | m-D13 | H | H | A-35 | H | H | H | H |
| 42148 | 1 | H | H | m-D13 | H | H | A-36 | H | H | H | H |
| 42149 | 1 | H | H | m-D13 | H | H | A-37 | H | H | H | H |
| 42150 | 1 | H | H | m-D13 | H | H | A-38 | H | H | H | H |
| 42151 | 1 | H | H | m-D13 | H | H | A-39 | H | H | H | H |
| 42152 | 1 | H | H | m-D13 | H | H | A-40 | H | H | H | H |
| 42153 | 1 | H | H | m-D13 | H | H | A-41 | H | H | H | H |
| 42154 | 1 | H | H | m-D13 | H | H | A-42 | H | H | H | H |
| 42155 | 1 | H | H | m-D13 | H | H | A-43 | H | H | H | H |
| 42156 | 1 | H | H | m-D13 | H | H | A-44 | H | H | H | H |
| 42157 | 1 | H | H | m-D13 | H | H | A-45 | H | H | H | H |
| 42158 | 1 | H | H | m-D13 | H | H | A-46 | H | H | H | H |
| 42159 | 1 | H | H | m-D13 | H | H | A-47 | H | H | H | H |
| 42160 | 1 | H | H | m-D14 | H | H | A-1 | H | H | H | H |
| 42161 | 1 | H | H | m-D14 | H | H | A-2 | H | H | H | H |
| 42162 | 1 | H | H | m-D14 | H | H | A-3 | H | H | H | H |
| 42163 | 1 | H | H | m-D14 | H | H | A-4 | H | H | H | H |
| 42164 | 1 | H | H | m-D14 | H | H | A-5 | H | H | H | H |
| 42165 | 1 | H | H | m-D14 | H | H | A-6 | H | H | H | H |
| 42166 | 1 | H | H | m-D14 | H | H | A-7 | H | H | H | H |
| 42167 | 1 | H | H | m-D14 | H | H | A-8 | H | H | H | H |
| 42168 | 1 | H | H | m-D14 | H | H | A-9 | H | H | H | H |
| 42169 | 1 | H | H | m-D14 | H | H | A-10 | H | H | H | H |
| 42170 | 1 | H | H | m-D14 | H | H | A-11 | H | H | H | H |
| 42171 | 1 | H | H | m-D14 | H | H | A-12 | H | H | H | H |
| 42172 | 1 | H | H | m-D14 | H | H | A-13 | H | H | H | H |
| 42173 | 1 | H | H | m-D14 | H | H | A-14 | H | H | H | H |
| 42174 | 1 | H | H | m-D14 | H | H | A-15 | H | H | H | H |
| 42175 | 1 | H | H | m-D14 | H | H | A-16 | H | H | H | H |
| 42176 | 1 | H | H | m-D14 | H | H | A-17 | H | H | H | H |
| 42177 | 1 | H | H | m-D14 | H | H | A-18 | H | H | H | H |
| 42178 | 1 | H | H | m-D14 | H | H | A-19 | H | H | H | H |
| 42179 | 1 | H | H | m-D14 | H | H | A-20 | H | H | H | H |
| 42180 | 1 | H | H | m-D14 | H | H | A-21 | H | H | H | H |
| 42181 | 1 | H | H | m-D14 | H | H | A-22 | H | H | H | H |
| 42182 | 1 | H | H | m-D14 | H | H | A-23 | H | H | H | H |
| 42183 | 1 | H | H | m-D14 | H | H | A-24 | H | H | H | H |
| 42184 | 1 | H | H | m-D14 | H | H | A-25 | H | H | H | H |
| 42185 | 1 | H | H | m-D14 | H | H | A-26 | H | H | H | H |
| 42186 | 1 | H | H | m-D14 | H | H | A-27 | H | H | H | H |
| 42187 | 1 | H | H | m-D14 | H | H | A-28 | H | H | H | H |
| 42188 | 1 | H | H | m-D14 | H | H | A-29 | H | H | H | H |
| 42189 | 1 | H | H | m-D14 | H | H | A-30 | H | H | H | H |
| 42190 | 1 | H | H | m-D14 | H | H | A-31 | H | H | H | H |
| 42191 | 1 | H | H | m-D14 | H | H | A-32 | H | H | H | H |
| 42192 | 1 | H | H | m-D14 | H | H | A-33 | H | H | H | H |
| 42193 | 1 | H | H | m-D14 | H | H | A-34 | H | H | H | H |
| 42194 | 1 | H | H | m-D14 | H | H | A-35 | H | H | H | H |
| 42195 | 1 | H | H | m-D14 | H | H | A-36 | H | H | H | H |
| 42196 | 1 | H | H | m-D14 | H | H | A-37 | H | H | H | H |
| 42197 | 1 | H | H | m-D14 | H | H | A-38 | H | H | H | H |
| 42198 | 1 | H | H | m-D14 | H | H | A-39 | H | H | H | H |
| 42199 | 1 | H | H | m-D14 | H | H | A-40 | H | H | H | H |
| 42200 | 1 | H | H | m-D14 | H | H | A-41 | H | H | H | H |
| 42201 | 1 | H | H | m-D14 | H | H | A-42 | H | H | H | H |
| 42202 | 1 | H | H | m-D14 | H | H | A-43 | H | H | H | H |
| 42203 | 1 | H | H | m-D14 | H | H | A-44 | H | H | H | H |
| 42204 | 1 | H | H | m-D14 | H | H | A-45 | H | H | H | H |
| 42205 | 1 | H | H | m-D14 | H | H | A-46 | H | H | H | H |
| 42206 | 1 | H | H | m-D14 | H | H | A-47 | H | H | H | H |
| 42207 | 1 | H | H | A-1 | H | H | m-D1 | H | H | H | H |
| 42208 | 1 | H | H | A-2 | H | H | m-D1 | H | H | H | H |
| 42209 | 1 | H | H | A-3 | H | H | m-D1 | H | H | H | H |
| 42210 | 1 | H | H | A-4 | H | H | m-D1 | H | H | H | H |
| 42211 | 1 | H | H | A-5 | H | H | m-D1 | H | H | H | H |
| 42212 | 1 | H | H | A-6 | H | H | m-D1 | H | H | H | H |
| 42213 | 1 | H | H | A-7 | H | H | m-D1 | H | H | H | H |
| 42214 | 1 | H | H | A-8 | H | H | m-D1 | H | H | H | H |
| 42215 | 1 | H | H | A-9 | H | H | m-D1 | H | H | H | H |
| 42216 | 1 | H | H | A-10 | H | H | m-D1 | H | H | H | H |
| 42217 | 1 | H | H | A-11 | H | H | m-D1 | H | H | H | H |
| 42218 | 1 | H | H | A-12 | H | H | m-D1 | H | H | H | H |
| 42219 | 1 | H | H | A-13 | H | H | m-D1 | H | H | H | H |
| 42220 | 1 | H | H | A-14 | H | H | m-D1 | H | H | H | H |
| 42221 | 1 | H | H | A-15 | H | H | m-D1 | H | H | H | H |
| 42222 | 1 | H | H | A-16 | H | H | m-D1 | H | H | H | H |
| 42223 | 1 | H | H | A-17 | H | H | m-D1 | H | H | H | H |
| 42224 | 1 | H | H | A-18 | H | H | m-D1 | H | H | H | H |
| 42225 | 1 | H | H | A-19 | H | H | m-D1 | H | H | H | H |
| 42226 | 1 | H | H | A-20 | H | H | m-D1 | H | H | H | H |
| 42227 | 1 | H | H | A-21 | H | H | m-D1 | H | H | H | H |
| 42228 | 1 | H | H | A-22 | H | H | m-D1 | H | H | H | H |
| 42229 | 1 | H | H | A-23 | H | H | m-D1 | H | H | H | H |
| 42230 | 1 | H | H | A-24 | H | H | m-D1 | H | H | H | H |
| 42231 | 1 | H | H | A-25 | H | H | m-D1 | H | H | H | H |
| 42232 | 1 | H | H | A-26 | H | H | m-D1 | H | H | H | H |
| 42233 | 1 | H | H | A-27 | H | H | m-D1 | H | H | H | H |
| 42234 | 1 | H | H | A-28 | H | H | m-D1 | H | H | H | H |
| 42235 | 1 | H | H | A-29 | H | H | m-D1 | H | H | H | H |
| 42236 | 1 | H | H | A-30 | H | H | m-D1 | H | H | H | H |
| 42237 | 1 | H | H | A-31 | H | H | m-D1 | H | H | H | H |
| 42238 | 1 | H | H | A-32 | H | H | m-D1 | H | H | H | H |
| 42239 | 1 | H | H | A-33 | H | H | m-D1 | H | H | H | H |
| 42240 | 1 | H | H | A-34 | H | H | m-D1 | H | H | H | H |
| 42241 | 1 | H | H | A-35 | H | H | m-D1 | H | H | H | H |
| 42242 | 1 | H | H | A-36 | H | H | m-D1 | H | H | H | H |
| 42243 | 1 | H | H | A-37 | H | H | m-D1 | H | H | H | H |
| 42244 | 1 | H | H | A-38 | H | H | m-D1 | H | H | H | H |
| 42245 | 1 | H | H | A-39 | H | H | m-D1 | H | H | H | H |
| 42246 | 1 | H | H | A-40 | H | H | m-D1 | H | H | H | H |
| 42247 | 1 | H | H | A-41 | H | H | m-D1 | H | H | H | H |
| 42248 | 1 | H | H | A-42 | H | H | m-D1 | H | H | H | H |
| 42249 | 1 | H | H | A-43 | H | H | m-D1 | H | H | H | H |
| 42250 | 1 | H | H | A-44 | H | H | m-D1 | H | H | H | H |
| 42251 | 1 | H | H | A-45 | H | H | m-D1 | H | H | H | H |
| 42252 | 1 | H | H | A-46 | H | H | m-D1 | H | H | H | H |
| 42253 | 1 | H | H | A-47 | H | H | m-D1 | H | H | H | H |
| 42254 | 1 | H | H | A-1 | H | H | m-D2 | H | H | H | H |
| 42255 | 1 | H | H | A-2 | H | H | m-D2 | H | H | H | H |
| 42256 | 1 | H | H | A-3 | H | H | m-D2 | H | H | H | H |
| 42257 | 1 | H | H | A-4 | H | H | m-D2 | H | H | H | H |
| 42258 | 1 | H | H | A-5 | H | H | m-D2 | H | H | H | H |
| 42259 | 1 | H | H | A-6 | H | H | m-D2 | H | H | H | H |
| 42260 | 1 | H | H | A-7 | H | H | m-D2 | H | H | H | H |
| 42261 | 1 | H | H | A-8 | H | H | m-D2 | H | H | H | H |
| 42262 | 1 | H | H | A-9 | H | H | m-D2 | H | H | H | H |
| 42263 | 1 | H | H | A-10 | H | H | m-D2 | H | H | H | H |
| 42264 | 1 | H | H | A-11 | H | H | m-D2 | H | H | H | H |
| 42265 | 1 | H | H | A-12 | H | H | m-D2 | H | H | H | H |
| 42266 | 1 | H | H | A-13 | H | H | m-D2 | H | H | H | H |
| 42267 | 1 | H | H | A-14 | H | H | m-D2 | H | H | H | H |
| 42268 | 1 | H | H | A-15 | H | H | m-D2 | H | H | H | H |
| 42269 | 1 | H | H | A-16 | H | H | m-D2 | H | H | H | H |
| 42270 | 1 | H | H | A-17 | H | H | m-D2 | H | H | H | H |
| 42271 | 1 | H | H | A-18 | H | H | m-D2 | H | H | H | H |
| 42272 | 1 | H | H | A-19 | H | H | m-D2 | H | H | H | H |
| 42273 | 1 | H | H | A-20 | H | H | m-D2 | H | H | H | H |
| 42274 | 1 | H | H | A-21 | H | H | m-D2 | H | H | H | H |
| 42275 | 1 | H | H | A-22 | H | H | m-D2 | H | H | H | H |
| 42276 | 1 | H | H | A-23 | H | H | m-D2 | H | H | H | H |
| 42277 | 1 | H | H | A-24 | H | H | m-D2 | H | H | H | H |
| 42278 | 1 | H | H | A-25 | H | H | m-D2 | H | H | H | H |
| 42279 | 1 | H | H | A-26 | H | H | m-D2 | H | H | H | H |
| 42280 | 1 | H | H | A-27 | H | H | m-D2 | H | H | H | H |
| 42281 | 1 | H | H | A-28 | H | H | m-D2 | H | H | H | H |
| 42282 | 1 | H | H | A-29 | H | H | m-D2 | H | H | H | H |
| 42283 | 1 | H | H | A-30 | H | H | m-D2 | H | H | H | H |
| 42284 | 1 | H | H | A-31 | H | H | m-D2 | H | H | H | H |
| 42285 | 1 | H | H | A-32 | H | H | m-D2 | H | H | H | H |
| 42286 | 1 | H | H | A-33 | H | H | m-D2 | H | H | H | H |
| 42287 | 1 | H | H | A-34 | H | H | m-D2 | H | H | H | H |
| 42288 | 1 | H | H | A-35 | H | H | m-D2 | H | H | H | H |
| 42289 | 1 | H | H | A-36 | H | H | m-D2 | H | H | H | H |
| 42290 | 1 | H | H | A-37 | H | H | m-D2 | H | H | H | H |
| 42291 | 1 | H | H | A-38 | H | H | m-D2 | H | H | H | H |
| 42292 | 1 | H | H | A-39 | H | H | m-D2 | H | H | H | H |
| 42293 | 1 | H | H | A-40 | H | H | m-D2 | H | H | H | H |
| 42294 | 1 | H | H | A-41 | H | H | m-D2 | H | H | H | H |
| 42295 | 1 | H | H | A-42 | H | H | m-D2 | H | H | H | H |
| 42296 | 1 | H | H | A-43 | H | H | m-D2 | H | H | H | H |
| 42297 | 1 | H | H | A-44 | H | H | m-D2 | H | H | H | H |
| 42298 | 1 | H | H | A-45 | H | H | m-D2 | H | H | H | H |
| 42299 | 1 | H | H | A-46 | H | H | m-D2 | H | H | H | H |
| 42300 | 1 | H | H | A-47 | H | H | m-D2 | H | H | H | H |
| 42301 | 1 | H | H | A-1 | H | H | m-D3 | H | H | H | H |
| 42302 | 1 | H | H | A-2 | H | H | m-D3 | H | H | H | H |
| 42303 | 1 | H | H | A-3 | H | H | m-D3 | H | H | H | H |
| 42304 | 1 | H | H | A-4 | H | H | m-D3 | H | H | H | H |
| 42305 | 1 | H | H | A-5 | H | H | m-D3 | H | H | H | H |
| 42306 | 1 | H | H | A-6 | H | H | m-D3 | H | H | H | H |
| 42307 | 1 | H | H | A-7 | H | H | m-D3 | H | H | H | H |
| 42308 | 1 | H | H | A-8 | H | H | m-D3 | H | H | H | H |
| 42309 | 1 | H | H | A-9 | H | H | m-D3 | H | H | H | H |
| 42310 | 1 | H | H | A-10 | H | H | m-D3 | H | H | H | H |
| 42311 | 1 | H | H | A-11 | H | H | m-D3 | H | H | H | H |
| 42312 | 1 | H | H | A-12 | H | H | m-D3 | H | H | H | H |
| 42313 | 1 | H | H | A-13 | H | H | m-D3 | H | H | H | H |
| 42314 | 1 | H | H | A-14 | H | H | m-D3 | H | H | H | H |
| 42315 | 1 | H | H | A-15 | H | H | m-D3 | H | H | H | H |
| 42316 | 1 | H | H | A-16 | H | H | m-D3 | H | H | H | H |
| 42317 | 1 | H | H | A-17 | H | H | m-D3 | H | H | H | H |
| 42318 | 1 | H | H | A-18 | H | H | m-D3 | H | H | H | H |
| 42319 | 1 | H | H | A-19 | H | H | m-D3 | H | H | H | H |
| 42320 | 1 | H | H | A-20 | H | H | m-D3 | H | H | H | H |
| 42321 | 1 | H | H | A-21 | H | H | m-D3 | H | H | H | H |
| 42322 | 1 | H | H | A-22 | H | H | m-D3 | H | H | H | H |
| 42323 | 1 | H | H | A-23 | H | H | m-D3 | H | H | H | H |
| 42324 | 1 | H | H | A-24 | H | H | m-D3 | H | H | H | H |
| 42325 | 1 | H | H | A-25 | H | H | m-D3 | H | H | H | H |
| 42326 | 1 | H | H | A-26 | H | H | m-D3 | H | H | H | H |
| 42327 | 1 | H | H | A-27 | H | H | m-D3 | H | H | H | H |
| 42328 | 1 | H | H | A-28 | H | H | m-D3 | H | H | H | H |
| 42329 | 1 | H | H | A-29 | H | H | m-D3 | H | H | H | H |
| 42330 | 1 | H | H | A-30 | H | H | m-D3 | H | H | H | H |
| 42331 | 1 | H | H | A-31 | H | H | m-D3 | H | H | H | H |
| 42332 | 1 | H | H | A-32 | H | H | m-D3 | H | H | H | H |
| 42333 | 1 | H | H | A-33 | H | H | m-D3 | H | H | H | H |
| 42334 | 1 | H | H | A-34 | H | H | m-D3 | H | H | H | H |
| 42335 | 1 | H | H | A-35 | H | H | m-D3 | H | H | H | H |
| 42336 | 1 | H | H | A-36 | H | H | m-D3 | H | H | H | H |
| 42337 | 1 | H | H | A-37 | H | H | m-D3 | H | H | H | H |
| 42338 | 1 | H | H | A-38 | H | H | m-D3 | H | H | H | H |
| 42339 | 1 | H | H | A-39 | H | H | m-D3 | H | H | H | H |
| 42340 | 1 | H | H | A-40 | H | H | m-D3 | H | H | H | H |
| 42341 | 1 | H | H | A-41 | H | H | m-D3 | H | H | H | H |
| 42342 | 1 | H | H | A-42 | H | H | m-D3 | H | H | H | H |
| 42343 | 1 | H | H | A-43 | H | H | m-D3 | H | H | H | H |
| 42344 | 1 | H | H | A-44 | H | H | m-D3 | H | H | H | H |
| 42345 | 1 | H | H | A-45 | H | H | m-D3 | H | H | H | H |
| 42346 | 1 | H | H | A-46 | H | H | m-D3 | H | H | H | H |
| 42347 | 1 | H | H | A-47 | H | H | m-D3 | H | H | H | H |
| 42348 | 1 | H | H | A-1 | H | H | m-D4 | H | H | H | H |
| 42349 | 1 | H | H | A-2 | H | H | m-D4 | H | H | H | H |
| 42350 | 1 | H | H | A-3 | H | H | m-D4 | H | H | H | H |
| 42351 | 1 | H | H | A-4 | H | H | m-D4 | H | H | H | H |
| 42352 | 1 | H | H | A-5 | H | H | m-D4 | H | H | H | H |
| 42353 | 1 | H | H | A-6 | H | H | m-D4 | H | H | H | H |
| 42354 | 1 | H | H | A-7 | H | H | m-D4 | H | H | H | H |
| 42355 | 1 | H | H | A-8 | H | H | m-D4 | H | H | H | H |
| 42356 | 1 | H | H | A-9 | H | H | m-D4 | H | H | H | H |
| 42357 | 1 | H | H | A-10 | H | H | m-D4 | H | H | H | H |
| 42358 | 1 | H | H | A-11 | H | H | m-D4 | H | H | H | H |
| 42359 | 1 | H | H | A-12 | H | H | m-D4 | H | H | H | H |
| 42360 | 1 | H | H | A-13 | H | H | m-D4 | H | H | H | H |
| 42361 | 1 | H | H | A-14 | H | H | m-D4 | H | H | H | H |
| 42362 | 1 | H | H | A-15 | H | H | m-D4 | H | H | H | H |
| 42363 | 1 | H | H | A-16 | H | H | m-D4 | H | H | H | H |
| 42364 | 1 | H | H | A-17 | H | H | m-D4 | H | H | H | H |
| 42365 | 1 | H | H | A-18 | H | H | m-D4 | H | H | H | H |
| 42366 | 1 | H | H | A-19 | H | H | m-D4 | H | H | H | H |
| 42367 | 1 | H | H | A-20 | H | H | m-D4 | H | H | H | H |
| 42368 | 1 | H | H | A-21 | H | H | m-D4 | H | H | H | H |
| 42369 | 1 | H | H | A-22 | H | H | m-D4 | H | H | H | H |
| 42370 | 1 | H | H | A-23 | H | H | m-D4 | H | H | H | H |
| 42371 | 1 | H | H | A-24 | H | H | m-D4 | H | H | H | H |
| 42372 | 1 | H | H | A-25 | H | H | m-D4 | H | H | H | H |
| 42373 | 1 | H | H | A-26 | H | H | m-D4 | H | H | H | H |
| 42374 | 1 | H | H | A-27 | H | H | m-D4 | H | H | H | H |
| 42375 | 1 | H | H | A-28 | H | H | m-D4 | H | H | H | H |
| 42376 | 1 | H | H | A-29 | H | H | m-D4 | H | H | H | H |
| 42377 | 1 | H | H | A-30 | H | H | m-D4 | H | H | H | H |
| 42378 | 1 | H | H | A-31 | H | H | m-D4 | H | H | H | H |
| 42379 | 1 | H | H | A-32 | H | H | m-D4 | H | H | H | H |
| 42380 | 1 | H | H | A-33 | H | H | m-D4 | H | H | H | H |
| 42381 | 1 | H | H | A-34 | H | H | m-D4 | H | H | H | H |
| 42382 | 1 | H | H | A-35 | H | H | m-D4 | H | H | H | H |
| 42383 | 1 | H | H | A-36 | H | H | m-D4 | H | H | H | H |
| 42384 | 1 | H | H | A-37 | H | H | m-D4 | H | H | H | H |
| 42385 | 1 | H | H | A-38 | H | H | m-D4 | H | H | H | H |
| 42386 | 1 | H | H | A-39 | H | H | m-D4 | H | H | H | H |
| 42387 | 1 | H | H | A-40 | H | H | m-D4 | H | H | H | H |
| 42388 | 1 | H | H | A-41 | H | H | m-D4 | H | H | H | H |
| 42389 | 1 | H | H | A-42 | H | H | m-D4 | H | H | H | H |
| 42390 | 1 | H | H | A-43 | H | H | m-D4 | H | H | H | H |
| 42391 | 1 | H | H | A-44 | H | H | m-D4 | H | H | H | H |
| 42392 | 1 | H | H | A-45 | H | H | m-D4 | H | H | H | H |
| 42393 | 1 | H | H | A-46 | H | H | m-D4 | H | H | H | H |
| 42394 | 1 | H | H | A-47 | H | H | m-D4 | H | H | H | H |
| 42395 | 1 | H | H | A-1 | H | H | m-D5 | H | H | H | H |
| 42396 | 1 | H | H | A-2 | H | H | m-D5 | H | H | H | H |
| 42397 | 1 | H | H | A-3 | H | H | m-D5 | H | H | H | H |
| 42398 | 1 | H | H | A-4 | H | H | m-D5 | H | H | H | H |
| 42399 | 1 | H | H | A-5 | H | H | m-D5 | H | H | H | H |
| 42400 | 1 | H | H | A-6 | H | H | m-D5 | H | H | H | H |
| 42401 | 1 | H | H | A-7 | H | H | m-D5 | H | H | H | H |
| 42402 | 1 | H | H | A-8 | H | H | m-D5 | H | H | H | H |
| 42403 | 1 | H | H | A-9 | H | H | m-D5 | H | H | H | H |
| 42404 | 1 | H | H | A-10 | H | H | m-D5 | H | H | H | H |
| 42405 | 1 | H | H | A-11 | H | H | m-D5 | H | H | H | H |
| 42406 | 1 | H | H | A-12 | H | H | m-D5 | H | H | H | H |
| 42407 | 1 | H | H | A-13 | H | H | m-D5 | H | H | H | H |
| 42408 | 1 | H | H | A-14 | H | H | m-D5 | H | H | H | H |
| 42409 | 1 | H | H | A-15 | H | H | m-D5 | H | H | H | H |
| 42410 | 1 | H | H | A-16 | H | H | m-D5 | H | H | H | H |
| 42411 | 1 | H | H | A-17 | H | H | m-D5 | H | H | H | H |
| 42412 | 1 | H | H | A-18 | H | H | m-D5 | H | H | H | H |
| 42413 | 1 | H | H | A-19 | H | H | m-D5 | H | H | H | H |
| 42414 | 1 | H | H | A-20 | H | H | m-D5 | H | H | H | H |
| 42415 | 1 | H | H | A-21 | H | H | m-D5 | H | H | H | H |
| 42416 | 1 | H | H | A-22 | H | H | m-D5 | H | H | H | H |
| 42417 | 1 | H | H | A-23 | H | H | m-D5 | H | H | H | H |
| 42418 | 1 | H | H | A-24 | H | H | m-D5 | H | H | H | H |
| 42419 | 1 | H | H | A-25 | H | H | m-D5 | H | H | H | H |
| 42420 | 1 | H | H | A-26 | H | H | m-D5 | H | H | H | H |
| 42421 | 1 | H | H | A-27 | H | H | m-D5 | H | H | H | H |
| 42422 | 1 | H | H | A-28 | H | H | m-D5 | H | H | H | H |
| 42423 | 1 | H | H | A-29 | H | H | m-D5 | H | H | H | H |
| 42424 | 1 | H | H | A-30 | H | H | m-D5 | H | H | H | H |
| 42425 | 1 | H | H | A-31 | H | H | m-D5 | H | H | H | H |
| 42426 | 1 | H | H | A-32 | H | H | m-D5 | H | H | H | H |
| 42427 | 1 | H | H | A-33 | H | H | m-D5 | H | H | H | H |
| 42428 | 1 | H | H | A-34 | H | H | m-D5 | H | H | H | H |
| 42429 | 1 | H | H | A-35 | H | H | m-D5 | H | H | H | H |
| 42430 | 1 | H | H | A-36 | H | H | m-D5 | H | H | H | H |
| 42431 | 1 | H | H | A-37 | H | H | m-D5 | H | H | H | H |
| 42432 | 1 | H | H | A-38 | H | H | m-D5 | H | H | H | H |
| 42433 | 1 | H | H | A-39 | H | H | m-D5 | H | H | H | H |
| 42434 | 1 | H | H | A-40 | H | H | m-D5 | H | H | H | H |
| 42435 | 1 | H | H | A-41 | H | H | m-D5 | H | H | H | H |
| 42436 | 1 | H | H | A-42 | H | H | m-D5 | H | H | H | H |
| 42437 | 1 | H | H | A-43 | H | H | m-D5 | H | H | H | H |
| 42438 | 1 | H | H | A-44 | H | H | m-D5 | H | H | H | H |
| 42439 | 1 | H | H | A-45 | H | H | m-D5 | H | H | H | H |
| 42440 | 1 | H | H | A-46 | H | H | m-D5 | H | H | H | H |
| 42441 | 1 | H | H | A-47 | H | H | m-D5 | H | H | H | H |
| 42442 | 1 | H | H | A-1 | H | H | m-D6 | H | H | H | H |
| 42443 | 1 | H | H | A-2 | H | H | m-D6 | H | H | H | H |
| 42444 | 1 | H | H | A-3 | H | H | m-D6 | H | H | H | H |
| 42445 | 1 | H | H | A-4 | H | H | m-D6 | H | H | H | H |
| 42446 | 1 | H | H | A-5 | H | H | m-D6 | H | H | H | H |
| 42447 | 1 | H | H | A-6 | H | H | m-D6 | H | H | H | H |
| 42448 | 1 | H | H | A-7 | H | H | m-D6 | H | H | H | H |
| 42449 | 1 | H | H | A-8 | H | H | m-D6 | H | H | H | H |
| 42450 | 1 | H | H | A-9 | H | H | m-D6 | H | H | H | H |
| 42451 | 1 | H | H | A-10 | H | H | m-D6 | H | H | H | H |
| 42452 | 1 | H | H | A-11 | H | H | m-D6 | H | H | H | H |
| 42453 | 1 | H | H | A-12 | H | H | m-D6 | H | H | H | H |
| 42454 | 1 | H | H | A-13 | H | H | m-D6 | H | H | H | H |
| 42455 | 1 | H | H | A-14 | H | H | m-D6 | H | H | H | H |
| 42456 | 1 | H | H | A-15 | H | H | m-D6 | H | H | H | H |
| 42457 | 1 | H | H | A-16 | H | H | m-D6 | H | H | H | H |
| 42458 | 1 | H | H | A-17 | H | H | m-D6 | H | H | H | H |
| 42459 | 1 | H | H | A-18 | H | H | m-D6 | H | H | H | H |
| 42460 | 1 | H | H | A-19 | H | H | m-D6 | H | H | H | H |
| 42461 | 1 | H | H | A-20 | H | H | m-D6 | H | H | H | H |
| 42462 | 1 | H | H | A-21 | H | H | m-D6 | H | H | H | H |
| 42463 | 1 | H | H | A-22 | H | H | m-D6 | H | H | H | H |
| 42464 | 1 | H | H | A-23 | H | H | m-D6 | H | H | H | H |
| 42465 | 1 | H | H | A-24 | H | H | m-D6 | H | H | H | H |
| 42466 | 1 | H | H | A-25 | H | H | m-D6 | H | H | H | H |
| 42467 | 1 | H | H | A-26 | H | H | m-D6 | H | H | H | H |
| 42468 | 1 | H | H | A-27 | H | H | m-D6 | H | H | H | H |
| 42469 | 1 | H | H | A-28 | H | H | m-D6 | H | H | H | H |
| 42470 | 1 | H | H | A-29 | H | H | m-D6 | H | H | H | H |
| 42471 | 1 | H | H | A-30 | H | H | m-D6 | H | H | H | H |
| 42472 | 1 | H | H | A-31 | H | H | m-D6 | H | H | H | H |
| 42473 | 1 | H | H | A-32 | H | H | m-D6 | H | H | H | H |
| 42474 | 1 | H | H | A-33 | H | H | m-D6 | H | H | H | H |
| 42475 | 1 | H | H | A-34 | H | H | m-D6 | H | H | H | H |
| 42476 | 1 | H | H | A-35 | H | H | m-D6 | H | H | H | H |
| 42477 | 1 | H | H | A-36 | H | H | m-D6 | H | H | H | H |
| 42478 | 1 | H | H | A-37 | H | H | m-D6 | H | H | H | H |
| 42479 | 1 | H | H | A-38 | H | H | m-D6 | H | H | H | H |
| 42480 | 1 | H | H | A-39 | H | H | m-D6 | H | H | H | H |
| 42481 | 1 | H | H | A-40 | H | H | m-D6 | H | H | H | H |
| 42482 | 1 | H | H | A-41 | H | H | m-D6 | H | H | H | H |
| 42483 | 1 | H | H | A-42 | H | H | m-D6 | H | H | H | H |
| 42484 | 1 | H | H | A-43 | H | H | m-D6 | H | H | H | H |
| 42485 | 1 | H | H | A-44 | H | H | m-D6 | H | H | H | H |
| 42486 | 1 | H | H | A-45 | H | H | m-D6 | H | H | H | H |
| 42487 | 1 | H | H | A-46 | H | H | m-D6 | H | H | H | H |
| 42488 | 1 | H | H | A-47 | H | H | m-D6 | H | H | H | H |
| 42489 | 1 | H | H | A-1 | H | H | m-D7 | H | H | H | H |
| 42490 | 1 | H | H | A-2 | H | H | m-D7 | H | H | H | H |
| 42491 | 1 | H | H | A-3 | H | H | m-D7 | H | H | H | H |
| 42492 | 1 | H | H | A-4 | H | H | m-D7 | H | H | H | H |
| 42493 | 1 | H | H | A-5 | H | H | m-D7 | H | H | H | H |
| 42494 | 1 | H | H | A-6 | H | H | m-D7 | H | H | H | H |
| 42495 | 1 | H | H | A-7 | H | H | m-D7 | H | H | H | H |
| 42496 | 1 | H | H | A-8 | H | H | m-D7 | H | H | H | H |
| 42497 | 1 | H | H | A-9 | H | H | m-D7 | H | H | H | H |
| 42498 | 1 | H | H | A-10 | H | H | m-D7 | H | H | H | H |
| 42499 | 1 | H | H | A-11 | H | H | m-D7 | H | H | H | H |
| 42500 | 1 | H | H | A-12 | H | H | m-D7 | H | H | H | H |
| 42501 | 1 | H | H | A-13 | H | H | m-D7 | H | H | H | H |
| 42502 | 1 | H | H | A-14 | H | H | m-D7 | H | H | H | H |
| 42503 | 1 | H | H | A-15 | H | H | m-D7 | H | H | H | H |
| 42504 | 1 | H | H | A-16 | H | H | m-D7 | H | H | H | H |
| 42505 | 1 | H | H | A-17 | H | H | m-D7 | H | H | H | H |
| 42506 | 1 | H | H | A-18 | H | H | m-D7 | H | H | H | H |
| 42507 | 1 | H | H | A-19 | H | H | m-D7 | H | H | H | H |
| 42508 | 1 | H | H | A-20 | H | H | m-D7 | H | H | H | H |
| 42509 | 1 | H | H | A-21 | H | H | m-D7 | H | H | H | H |
| 42510 | 1 | H | H | A-22 | H | H | m-D7 | H | H | H | H |
| 42511 | 1 | H | H | A-23 | H | H | m-D7 | H | H | H | H |
| 42512 | 1 | H | H | A-24 | H | H | m-D7 | H | H | H | H |
| 42513 | 1 | H | H | A-25 | H | H | m-D7 | H | H | H | H |
| 42514 | 1 | H | H | A-26 | H | H | m-D7 | H | H | H | H |
| 42515 | 1 | H | H | A-27 | H | H | m-D7 | H | H | H | H |
| 42516 | 1 | H | H | A-28 | H | H | m-D7 | H | H | H | H |
| 42517 | 1 | H | H | A-29 | H | H | m-D7 | H | H | H | H |
| 42518 | 1 | H | H | A-30 | H | H | m-D7 | H | H | H | H |
| 42519 | 1 | H | H | A-31 | H | H | m-D7 | H | H | H | H |
| 42520 | 1 | H | H | A-32 | H | H | m-D7 | H | H | H | H |
| 42521 | 1 | H | H | A-33 | H | H | m-D7 | H | H | H | H |
| 42522 | 1 | H | H | A-34 | H | H | m-D7 | H | H | H | H |
| 42523 | 1 | H | H | A-35 | H | H | m-D7 | H | H | H | H |
| 42524 | 1 | H | H | A-36 | H | H | m-D7 | H | H | H | H |
| 42525 | 1 | H | H | A-37 | H | H | m-D7 | H | H | H | H |
| 42526 | 1 | H | H | A-38 | H | H | m-D7 | H | H | H | H |
| 42527 | 1 | H | H | A-39 | H | H | m-D7 | H | H | H | H |
| 42528 | 1 | H | H | A-40 | H | H | m-D7 | H | H | H | H |
| 42529 | 1 | H | H | A-41 | H | H | m-D7 | H | H | H | H |
| 42530 | 1 | H | H | A-42 | H | H | m-D7 | H | H | H | H |
| 42531 | 1 | H | H | A-43 | H | H | m-D7 | H | H | H | H |
| 42532 | 1 | H | H | A-44 | H | H | m-D7 | H | H | H | H |
| 42533 | 1 | H | H | A-45 | H | H | m-D7 | H | H | H | H |
| 42534 | 1 | H | H | A-46 | H | H | m-D7 | H | H | H | H |
| 42535 | 1 | H | H | A-47 | H | H | m-D7 | H | H | H | H |
| 42536 | 1 | H | H | A-1 | H | H | m-D8 | H | H | H | H |
| 42537 | 1 | H | H | A-2 | H | H | m-D8 | H | H | H | H |
| 42538 | 1 | H | H | A-3 | H | H | m-D8 | H | H | H | H |
| 42539 | 1 | H | H | A-4 | H | H | m-D8 | H | H | H | H |
| 42540 | 1 | H | H | A-5 | H | H | m-D8 | H | H | H | H |
| 42541 | 1 | H | H | A-6 | H | H | m-D8 | H | H | H | H |
| 42542 | 1 | H | H | A-7 | H | H | m-D8 | H | H | H | H |
| 42543 | 1 | H | H | A-8 | H | H | m-D8 | H | H | H | H |
| 42544 | 1 | H | H | A-9 | H | H | m-D8 | H | H | H | H |
| 42545 | 1 | H | H | A-10 | H | H | m-D8 | H | H | H | H |
| 42546 | 1 | H | H | A-11 | H | H | m-D8 | H | H | H | H |
| 42547 | 1 | H | H | A-12 | H | H | m-D8 | H | H | H | H |
| 42548 | 1 | H | H | A-13 | H | H | m-D8 | H | H | H | H |
| 42549 | 1 | H | H | A-14 | H | H | m-D8 | H | H | H | H |
| 42550 | 1 | H | H | A-15 | H | H | m-D8 | H | H | H | H |
| 42551 | 1 | H | H | A-16 | H | H | m-D8 | H | H | H | H |
| 42552 | 1 | H | H | A-17 | H | H | m-D8 | H | H | H | H |
| 42553 | 1 | H | H | A-18 | H | H | m-D8 | H | H | H | H |
| 42554 | 1 | H | H | A-19 | H | H | m-D8 | H | H | H | H |
| 42555 | 1 | H | H | A-20 | H | H | m-D8 | H | H | H | H |
| 42556 | 1 | H | H | A-21 | H | H | m-D8 | H | H | H | H |
| 42557 | 1 | H | H | A-22 | H | H | m-D8 | H | H | H | H |
| 42558 | 1 | H | H | A-23 | H | H | m-D8 | H | H | H | H |
| 42559 | 1 | H | H | A-24 | H | H | m-D8 | H | H | H | H |
| 42560 | 1 | H | H | A-25 | H | H | m-D8 | H | H | H | H |
| 42561 | 1 | H | H | A-26 | H | H | m-D8 | H | H | H | H |
| 42562 | 1 | H | H | A-27 | H | H | m-D8 | H | H | H | H |
| 42563 | 1 | H | H | A-28 | H | H | m-D8 | H | H | H | H |
| 42564 | 1 | H | H | A-29 | H | H | m-D8 | H | H | H | H |
| 42565 | 1 | H | H | A-30 | H | H | m-D8 | H | H | H | H |
| 42566 | 1 | H | H | A-31 | H | H | m-D8 | H | H | H | H |
| 42567 | 1 | H | H | A-32 | H | H | m-D8 | H | H | H | H |
| 42568 | 1 | H | H | A-33 | H | H | m-D8 | H | H | H | H |
| 42569 | 1 | H | H | A-34 | H | H | m-D8 | H | H | H | H |
| 42570 | 1 | H | H | A-35 | H | H | m-D8 | H | H | H | H |
| 42571 | 1 | H | H | A-36 | H | H | m-D8 | H | H | H | H |
| 42572 | 1 | H | H | A-37 | H | H | m-D8 | H | H | H | H |
| 42573 | 1 | H | H | A-38 | H | H | m-D8 | H | H | H | H |
| 42574 | 1 | H | H | A-39 | H | H | m-D8 | H | H | H | H |
| 42575 | 1 | H | H | A-40 | H | H | m-D8 | H | H | H | H |
| 42576 | 1 | H | H | A-41 | H | H | m-D8 | H | H | H | H |
| 42577 | 1 | H | H | A-42 | H | H | m-D8 | H | H | H | H |
| 42578 | 1 | H | H | A-43 | H | H | m-D8 | H | H | H | H |
| 42579 | 1 | H | H | A-44 | H | H | m-D8 | H | H | H | H |
| 42580 | 1 | H | H | A-45 | H | H | m-D8 | H | H | H | H |
| 42581 | 1 | H | H | A-46 | H | H | m-D8 | H | H | H | H |
| 42582 | 1 | H | H | A-47 | H | H | m-D8 | H | H | H | H |
| 42583 | 1 | H | H | A-1 | H | H | m-D9 | H | H | H | H |
| 42584 | 1 | H | H | A-2 | H | H | m-D9 | H | H | H | H |
| 42585 | 1 | H | H | A-3 | H | H | m-D9 | H | H | H | H |
| 42586 | 1 | H | H | A-4 | H | H | m-D9 | H | H | H | H |
| 42587 | 1 | H | H | A-5 | H | H | m-D9 | H | H | H | H |
| 42588 | 1 | H | H | A-6 | H | H | m-D9 | H | H | H | H |
| 42589 | 1 | H | H | A-7 | H | H | m-D9 | H | H | H | H |
| 42590 | 1 | H | H | A-8 | H | H | m-D9 | H | H | H | H |
| 42591 | 1 | H | H | A-9 | H | H | m-D9 | H | H | H | H |
| 42592 | 1 | H | H | A-10 | H | H | m-D9 | H | H | H | H |
| 42593 | 1 | H | H | A-11 | H | H | m-D9 | H | H | H | H |
| 42594 | 1 | H | H | A-12 | H | H | m-D9 | H | H | H | H |
| 42595 | 1 | H | H | A-13 | H | H | m-D9 | H | H | H | H |
| 42596 | 1 | H | H | A-14 | H | H | m-D9 | H | H | H | H |
| 42597 | 1 | H | H | A-15 | H | H | m-D9 | H | H | H | H |
| 42598 | 1 | H | H | A-16 | H | H | m-D9 | H | H | H | H |
| 42599 | 1 | H | H | A-17 | H | H | m-D9 | H | H | H | H |
| 42600 | 1 | H | H | A-18 | H | H | m-D9 | H | H | H | H |
| 42601 | 1 | H | H | A-19 | H | H | m-D9 | H | H | H | H |
| 42602 | 1 | H | H | A-20 | H | H | m-D9 | H | H | H | H |
| 42603 | 1 | H | H | A-21 | H | H | m-D9 | H | H | H | H |
| 42604 | 1 | H | H | A-22 | H | H | m-D9 | H | H | H | H |
| 42605 | 1 | H | H | A-23 | H | H | m-D9 | H | H | H | H |
| 42606 | 1 | H | H | A-24 | H | H | m-D9 | H | H | H | H |
| 42607 | 1 | H | H | A-25 | H | H | m-D9 | H | H | H | H |
| 42608 | 1 | H | H | A-26 | H | H | m-D9 | H | H | H | H |
| 42609 | 1 | H | H | A-27 | H | H | m-D9 | H | H | H | H |
| 42610 | 1 | H | H | A-28 | H | H | m-D9 | H | H | H | H |
| 42611 | 1 | H | H | A-29 | H | H | m-D9 | H | H | H | H |
| 42612 | 1 | H | H | A-30 | H | H | m-D9 | H | H | H | H |
| 42613 | 1 | H | H | A-31 | H | H | m-D9 | H | H | H | H |
| 42614 | 1 | H | H | A-32 | H | H | m-D9 | H | H | H | H |
| 42615 | 1 | H | H | A-33 | H | H | m-D9 | H | H | H | H |
| 42616 | 1 | H | H | A-34 | H | H | m-D9 | H | H | H | H |
| 42617 | 1 | H | H | A-35 | H | H | m-D9 | H | H | H | H |
| 42618 | 1 | H | H | A-36 | H | H | m-D9 | H | H | H | H |
| 42619 | 1 | H | H | A-37 | H | H | m-D9 | H | H | H | H |
| 42620 | 1 | H | H | A-38 | H | H | m-D9 | H | H | H | H |
| 42621 | 1 | H | H | A-39 | H | H | m-D9 | H | H | H | H |
| 42622 | 1 | H | H | A-40 | H | H | m-D9 | H | H | H | H |
| 42623 | 1 | H | H | A-41 | H | H | m-D9 | H | H | H | H |
| 42624 | 1 | H | H | A-42 | H | H | m-D9 | H | H | H | H |
| 42625 | 1 | H | H | A-43 | H | H | m-D9 | H | H | H | H |
| 42626 | 1 | H | H | A-44 | H | H | m-D9 | H | H | H | H |
| 42627 | 1 | H | H | A-45 | H | H | m-D9 | H | H | H | H |
| 42628 | 1 | H | H | A-46 | H | H | m-D9 | H | H | H | H |
| 42629 | 1 | H | H | A-47 | H | H | m-D9 | H | H | H | H |
| 42630 | 1 | H | H | A-1 | H | H | m-D10 | H | H | H | H |
| 42631 | 1 | H | H | A-2 | H | H | m-D10 | H | H | H | H |
| 42632 | 1 | H | H | A-3 | H | H | m-D10 | H | H | H | H |
| 42633 | 1 | H | H | A-4 | H | H | m-D10 | H | H | H | H |
| 42634 | 1 | H | H | A-5 | H | H | m-D10 | H | H | H | H |
| 42635 | 1 | H | H | A-6 | H | H | m-D10 | H | H | H | H |
| 42636 | 1 | H | H | A-7 | H | H | m-D10 | H | H | H | H |
| 42637 | 1 | H | H | A-8 | H | H | m-D10 | H | H | H | H |
| 42638 | 1 | H | H | A-9 | H | H | m-D10 | H | H | H | H |
| 42639 | 1 | H | H | A-10 | H | H | m-D10 | H | H | H | H |
| 42640 | 1 | H | H | A-11 | H | H | m-D10 | H | H | H | H |
| 42641 | 1 | H | H | A-12 | H | H | m-D10 | H | H | H | H |
| 42642 | 1 | H | H | A-13 | H | H | m-D10 | H | H | H | H |
| 42643 | 1 | H | H | A-14 | H | H | m-D10 | H | H | H | H |
| 42644 | 1 | H | H | A-15 | H | H | m-D10 | H | H | H | H |
| 42645 | 1 | H | H | A-16 | H | H | m-D10 | H | H | H | H |
| 42646 | 1 | H | H | A-17 | H | H | m-D10 | H | H | H | H |
| 42647 | 1 | H | H | A-18 | H | H | m-D10 | H | H | H | H |
| 42648 | 1 | H | H | A-19 | H | H | m-D10 | H | H | H | H |
| 42649 | 1 | H | H | A-20 | H | H | m-D10 | H | H | H | H |
| 42650 | 1 | H | H | A-21 | H | H | m-D10 | H | H | H | H |
| 42651 | 1 | H | H | A-22 | H | H | m-D10 | H | H | H | H |
| 42652 | 1 | H | H | A-23 | H | H | m-D10 | H | H | H | H |
| 42653 | 1 | H | H | A-24 | H | H | m-D10 | H | H | H | H |
| 42654 | 1 | H | H | A-25 | H | H | m-D10 | H | H | H | H |
| 42655 | 1 | H | H | A-26 | H | H | m-D10 | H | H | H | H |
| 42656 | 1 | H | H | A-27 | H | H | m-D10 | H | H | H | H |
| 42657 | 1 | H | H | A-28 | H | H | m-D10 | H | H | H | H |
| 42658 | 1 | H | H | A-29 | H | H | m-D10 | H | H | H | H |
| 42659 | 1 | H | H | A-30 | H | H | m-D10 | H | H | H | H |
| 42660 | 1 | H | H | A-31 | H | H | m-D10 | H | H | H | H |
| 42661 | 1 | H | H | A-32 | H | H | m-D10 | H | H | H | H |
| 42662 | 1 | H | H | A-33 | H | H | m-D10 | H | H | H | H |
| 42663 | 1 | H | H | A-34 | H | H | m-D10 | H | H | H | H |
| 42664 | 1 | H | H | A-35 | H | H | m-D10 | H | H | H | H |
| 42665 | 1 | H | H | A-36 | H | H | m-D10 | H | H | H | H |
| 42666 | 1 | H | H | A-37 | H | H | m-D10 | H | H | H | H |
| 42667 | 1 | H | H | A-38 | H | H | m-D10 | H | H | H | H |
| 42668 | 1 | H | H | A-39 | H | H | m-D10 | H | H | H | H |
| 42669 | 1 | H | H | A-40 | H | H | m-D10 | H | H | H | H |
| 42670 | 1 | H | H | A-41 | H | H | m-D10 | H | H | H | H |
| 42671 | 1 | H | H | A-42 | H | H | m-D10 | H | H | H | H |
| 42672 | 1 | H | H | A-43 | H | H | m-D10 | H | H | H | H |
| 42673 | 1 | H | H | A-44 | H | H | m-D10 | H | H | H | H |
| 42674 | 1 | H | H | A-45 | H | H | m-D10 | H | H | H | H |
| 42675 | 1 | H | H | A-46 | H | H | m-D10 | H | H | H | H |
| 42676 | 1 | H | H | A-47 | H | H | m-D10 | H | H | H | H |
| 42677 | 1 | H | H | A-1 | H | H | m-D11 | H | H | H | H |
| 42678 | 1 | H | H | A-2 | H | H | m-D11 | H | H | H | H |
| 42679 | 1 | H | H | A-3 | H | H | m-D11 | H | H | H | H |
| 42680 | 1 | H | H | A-4 | H | H | m-D11 | H | H | H | H |
| 42681 | 1 | H | H | A-5 | H | H | m-D11 | H | H | H | H |
| 42682 | 1 | H | H | A-6 | H | H | m-D11 | H | H | H | H |
| 42683 | 1 | H | H | A-7 | H | H | m-D11 | H | H | H | H |
| 42684 | 1 | H | H | A-8 | H | H | m-D11 | H | H | H | H |
| 42685 | 1 | H | H | A-9 | H | H | m-D11 | H | H | H | H |
| 42686 | 1 | H | H | A-10 | H | H | m-D11 | H | H | H | H |
| 42687 | 1 | H | H | A-11 | H | H | m-D11 | H | H | H | H |
| 42688 | 1 | H | H | A-12 | H | H | m-D11 | H | H | H | H |
| 42689 | 1 | H | H | A-13 | H | H | m-D11 | H | H | H | H |
| 42690 | 1 | H | H | A-14 | H | H | m-D11 | H | H | H | H |
| 42691 | 1 | H | H | A-15 | H | H | m-D11 | H | H | H | H |
| 42692 | 1 | H | H | A-16 | H | H | m-D11 | H | H | H | H |
| 42693 | 1 | H | H | A-17 | H | H | m-D11 | H | H | H | H |
| 42694 | 1 | H | H | A-18 | H | H | m-D11 | H | H | H | H |
| 42695 | 1 | H | H | A-19 | H | H | m-D11 | H | H | H | H |
| 42696 | 1 | H | H | A-20 | H | H | m-D11 | H | H | H | H |
| 42697 | 1 | H | H | A-21 | H | H | m-D11 | H | H | H | H |
| 42698 | 1 | H | H | A-22 | H | H | m-D11 | H | H | H | H |
| 42699 | 1 | H | H | A-23 | H | H | m-D11 | H | H | H | H |
| 42700 | 1 | H | H | A-24 | H | H | m-D11 | H | H | H | H |
| 42701 | 1 | H | H | A-25 | H | H | m-D11 | H | H | H | H |
| 42702 | 1 | H | H | A-26 | H | H | m-D11 | H | H | H | H |
| 42703 | 1 | H | H | A-27 | H | H | m-D11 | H | H | H | H |
| 42704 | 1 | H | H | A-28 | H | H | m-D11 | H | H | H | H |
| 42705 | 1 | H | H | A-29 | H | H | m-D11 | H | H | H | H |
| 42706 | 1 | H | H | A-30 | H | H | m-D11 | H | H | H | H |
| 42707 | 1 | H | H | A-31 | H | H | m-D11 | H | H | H | H |
| 42708 | 1 | H | H | A-32 | H | H | m-D11 | H | H | H | H |
| 42709 | 1 | H | H | A-33 | H | H | m-D11 | H | H | H | H |
| 42710 | 1 | H | H | A-34 | H | H | m-D11 | H | H | H | H |
| 42711 | 1 | H | H | A-35 | H | H | m-D11 | H | H | H | H |
| 42712 | 1 | H | H | A-36 | H | H | m-D11 | H | H | H | H |
| 42713 | 1 | H | H | A-37 | H | H | m-D11 | H | H | H | H |
| 42714 | 1 | H | H | A-38 | H | H | m-D11 | H | H | H | H |
| 42715 | 1 | H | H | A-39 | H | H | m-D11 | H | H | H | H |
| 42716 | 1 | H | H | A-40 | H | H | m-D11 | H | H | H | H |
| 42717 | 1 | H | H | A-41 | H | H | m-D11 | H | H | H | H |
| 42718 | 1 | H | H | A-42 | H | H | m-D11 | H | H | H | H |
| 42719 | 1 | H | H | A-43 | H | H | m-D11 | H | H | H | H |
| 42720 | 1 | H | H | A-44 | H | H | m-D11 | H | H | H | H |
| 42721 | 1 | H | H | A-45 | H | H | m-D11 | H | H | H | H |
| 42722 | 1 | H | H | A-46 | H | H | m-D11 | H | H | H | H |
| 42723 | 1 | H | H | A-47 | H | H | m-D11 | H | H | H | H |
| 42724 | 1 | H | H | A-1 | H | H | m-D12 | H | H | H | H |
| 42725 | 1 | H | H | A-2 | H | H | m-D12 | H | H | H | H |
| 42726 | 1 | H | H | A-3 | H | H | m-D12 | H | H | H | H |
| 42727 | 1 | H | H | A-4 | H | H | m-D12 | H | H | H | H |
| 42728 | 1 | H | H | A-5 | H | H | m-D12 | H | H | H | H |
| 42729 | 1 | H | H | A-6 | H | H | m-D12 | H | H | H | H |
| 42730 | 1 | H | H | A-7 | H | H | m-D12 | H | H | H | H |
| 42731 | 1 | H | H | A-8 | H | H | m-D12 | H | H | H | H |
| 42732 | 1 | H | H | A-9 | H | H | m-D12 | H | H | H | H |
| 42733 | 1 | H | H | A-10 | H | H | m-D12 | H | H | H | H |
| 42734 | 1 | H | H | A-11 | H | H | m-D12 | H | H | H | H |
| 42735 | 1 | H | H | A-12 | H | H | m-D12 | H | H | H | H |
| 42736 | 1 | H | H | A-13 | H | H | m-D12 | H | H | H | H |
| 42737 | 1 | H | H | A-14 | H | H | m-D12 | H | H | H | H |
| 42738 | 1 | H | H | A-15 | H | H | m-D12 | H | H | H | H |
| 42739 | 1 | H | H | A-16 | H | H | m-D12 | H | H | H | H |
| 42740 | 1 | H | H | A-17 | H | H | m-D12 | H | H | H | H |
| 42741 | 1 | H | H | A-18 | H | H | m-D12 | H | H | H | H |
| 42742 | 1 | H | H | A-19 | H | H | m-D12 | H | H | H | H |
| 42743 | 1 | H | H | A-20 | H | H | m-D12 | H | H | H | H |
| 42744 | 1 | H | H | A-21 | H | H | m-D12 | H | H | H | H |
| 42745 | 1 | H | H | A-22 | H | H | m-D12 | H | H | H | H |
| 42746 | 1 | H | H | A-23 | H | H | m-D12 | H | H | H | H |
| 42747 | 1 | H | H | A-24 | H | H | m-D12 | H | H | H | H |
| 42748 | 1 | H | H | A-25 | H | H | m-D12 | H | H | H | H |
| 42749 | 1 | H | H | A-26 | H | H | m-D12 | H | H | H | H |
| 42750 | 1 | H | H | A-27 | H | H | m-D12 | H | H | H | H |
| 42751 | 1 | H | H | A-28 | H | H | m-D12 | H | H | H | H |
| 42752 | 1 | H | H | A-29 | H | H | m-D12 | H | H | H | H |
| 42753 | 1 | H | H | A-30 | H | H | m-D12 | H | H | H | H |
| 42754 | 1 | H | H | A-31 | H | H | m-D12 | H | H | H | H |
| 42755 | 1 | H | H | A-32 | H | H | m-D12 | H | H | H | H |
| 42756 | 1 | H | H | A-33 | H | H | m-D12 | H | H | H | H |
| 42757 | 1 | H | H | A-34 | H | H | m-D12 | H | H | H | H |
| 42758 | 1 | H | H | A-35 | H | H | m-D12 | H | H | H | H |
| 42759 | 1 | H | H | A-36 | H | H | m-D12 | H | H | H | H |
| 42760 | 1 | H | H | A-37 | H | H | m-D12 | H | H | H | H |
| 42761 | 1 | H | H | A-38 | H | H | m-D12 | H | H | H | H |
| 42762 | 1 | H | H | A-39 | H | H | m-D12 | H | H | H | H |
| 42763 | 1 | H | H | A-40 | H | H | m-D12 | H | H | H | H |
| 42764 | 1 | H | H | A-41 | H | H | m-D12 | H | H | H | H |
| 42765 | 1 | H | H | A-42 | H | H | m-D12 | H | H | H | H |
| 42766 | 1 | H | H | A-43 | H | H | m-D12 | H | H | H | H |
| 42767 | 1 | H | H | A-44 | H | H | m-D12 | H | H | H | H |
| 42768 | 1 | H | H | A-45 | H | H | m-D12 | H | H | H | H |
| 42769 | 1 | H | H | A-46 | H | H | m-D12 | H | H | H | H |
| 42770 | 1 | H | H | A-47 | H | H | m-D12 | H | H | H | H |
| 42771 | 1 | H | H | A-1 | H | H | m-D13 | H | H | H | H |
| 42772 | 1 | H | H | A-2 | H | H | m-D13 | H | H | H | H |
| 42773 | 1 | H | H | A-3 | H | H | m-D13 | H | H | H | H |
| 42774 | 1 | H | H | A-4 | H | H | m-D13 | H | H | H | H |
| 42775 | 1 | H | H | A-5 | H | H | m-D13 | H | H | H | H |
| 42776 | 1 | H | H | A-6 | H | H | m-D13 | H | H | H | H |
| 42777 | 1 | H | H | A-7 | H | H | m-D13 | H | H | H | H |
| 42778 | 1 | H | H | A-8 | H | H | m-D13 | H | H | H | H |
| 42779 | 1 | H | H | A-9 | H | H | m-D13 | H | H | H | H |
| 42780 | 1 | H | H | A-10 | H | H | m-D13 | H | H | H | H |
| 42781 | 1 | H | H | A-11 | H | H | m-D13 | H | H | H | H |
| 42782 | 1 | H | H | A-12 | H | H | m-D13 | H | H | H | H |
| 42783 | 1 | H | H | A-13 | H | H | m-D13 | H | H | H | H |
| 42784 | 1 | H | H | A-14 | H | H | m-D13 | H | H | H | H |
| 42785 | 1 | H | H | A-15 | H | H | m-D13 | H | H | H | H |
| 42786 | 1 | H | H | A-16 | H | H | m-D13 | H | H | H | H |
| 42787 | 1 | H | H | A-17 | H | H | m-D13 | H | H | H | H |
| 42788 | 1 | H | H | A-18 | H | H | m-D13 | H | H | H | H |
| 42789 | 1 | H | H | A-19 | H | H | m-D13 | H | H | H | H |
| 42790 | 1 | H | H | A-20 | H | H | m-D13 | H | H | H | H |
| 42791 | 1 | H | H | A-21 | H | H | m-D13 | H | H | H | H |
| 42792 | 1 | H | H | A-22 | H | H | m-D13 | H | H | H | H |
| 42793 | 1 | H | H | A-23 | H | H | m-D13 | H | H | H | H |
| 42794 | 1 | H | H | A-24 | H | H | m-D13 | H | H | H | H |
| 42795 | 1 | H | H | A-25 | H | H | m-D13 | H | H | H | H |
| 42796 | 1 | H | H | A-26 | H | H | m-D13 | H | H | H | H |
| 42797 | 1 | H | H | A-27 | H | H | m-D13 | H | H | H | H |
| 42798 | 1 | H | H | A-28 | H | H | m-D13 | H | H | H | H |
| 42799 | 1 | H | H | A-29 | H | H | m-D13 | H | H | H | H |
| 42800 | 1 | H | H | A-30 | H | H | m-D13 | H | H | H | H |
| 42801 | 1 | H | H | A-31 | H | H | m-D13 | H | H | H | H |
| 42802 | 1 | H | H | A-32 | H | H | m-D13 | H | H | H | H |
| 42803 | 1 | H | H | A-33 | H | H | m-D13 | H | H | H | H |
| 42804 | 1 | H | H | A-34 | H | H | m-D13 | H | H | H | H |
| 42805 | 1 | H | H | A-35 | H | H | m-D13 | H | H | H | H |
| 42806 | 1 | H | H | A-36 | H | H | m-D13 | H | H | H | H |
| 42807 | 1 | H | H | A-37 | H | H | m-D13 | H | H | H | H |
| 42808 | 1 | H | H | A-38 | H | H | m-D13 | H | H | H | H |
| 42809 | 1 | H | H | A-39 | H | H | m-D13 | H | H | H | H |
| 42810 | 1 | H | H | A-40 | H | H | m-D13 | H | H | H | H |
| 42811 | 1 | H | H | A-41 | H | H | m-D13 | H | H | H | H |
| 42812 | 1 | H | H | A-42 | H | H | m-D13 | H | H | H | H |
| 42813 | 1 | H | H | A-43 | H | H | m-D13 | H | H | H | H |
| 42814 | 1 | H | H | A-44 | H | H | m-D13 | H | H | H | H |
| 42815 | 1 | H | H | A-45 | H | H | m-D13 | H | H | H | H |
| 42816 | 1 | H | H | A-46 | H | H | m-D13 | H | H | H | H |
| 42817 | 1 | H | H | A-47 | H | H | m-D13 | H | H | H | H |
| 42818 | 1 | H | H | A-1 | H | H | m-D14 | H | H | H | H |
| 42819 | 1 | H | H | A-2 | H | H | m-D14 | H | H | H | H |
| 42820 | 1 | H | H | A-3 | H | H | m-D14 | H | H | H | H |
| 42821 | 1 | H | H | A-4 | H | H | m-D14 | H | H | H | H |
| 42822 | 1 | H | H | A-5 | H | H | m-D14 | H | H | H | H |
| 42823 | 1 | H | H | A-6 | H | H | m-D14 | H | H | H | H |
| 42824 | 1 | H | H | A-7 | H | H | m-D14 | H | H | H | H |
| 42825 | 1 | H | H | A-8 | H | H | m-D14 | H | H | H | H |
| 42826 | 1 | H | H | A-9 | H | H | m-D14 | H | H | H | H |
| 42827 | 1 | H | H | A-10 | H | H | m-D14 | H | H | H | H |
| 42828 | 1 | H | H | A-11 | H | H | m-D14 | H | H | H | H |
| 42829 | 1 | H | H | A-12 | H | H | m-D14 | H | H | H | H |
| 42830 | 1 | H | H | A-13 | H | H | m-D14 | H | H | H | H |
| 42831 | 1 | H | H | A-14 | H | H | m-D14 | H | H | H | H |
| 42832 | 1 | H | H | A-15 | H | H | m-D14 | H | H | H | H |
| 42833 | 1 | H | H | A-16 | H | H | m-D14 | H | H | H | H |
| 42834 | 1 | H | H | A-17 | H | H | m-D14 | H | H | H | H |
| 42835 | 1 | H | H | A-18 | H | H | m-D14 | H | H | H | H |
| 42836 | 1 | H | H | A-19 | H | H | m-D14 | H | H | H | H |
| 42837 | 1 | H | H | A-20 | H | H | m-D14 | H | H | H | H |
| 42838 | 1 | H | H | A-21 | H | H | m-D14 | H | H | H | H |
| 42839 | 1 | H | H | A-22 | H | H | m-D14 | H | H | H | H |
| 42840 | 1 | H | H | A-23 | H | H | m-D14 | H | H | H | H |
| 42841 | 1 | H | H | A-24 | H | H | m-D14 | H | H | H | H |
| 42842 | 1 | H | H | A-25 | H | H | m-D14 | H | H | H | H |
| 42843 | 1 | H | H | A-26 | H | H | m-D14 | H | H | H | H |
| 42844 | 1 | H | H | A-27 | H | H | m-D14 | H | H | H | H |
| 42845 | 1 | H | H | A-28 | H | H | m-D14 | H | H | H | H |
| 42846 | 1 | H | H | A-29 | H | H | m-D14 | H | H | H | H |
| 42847 | 1 | H | H | A-30 | H | H | m-D14 | H | H | H | H |
| 42848 | 1 | H | H | A-31 | H | H | m-D14 | H | H | H | H |
| 42849 | 1 | H | H | A-32 | H | H | m-D14 | H | H | H | H |
| 42850 | 1 | H | H | A-33 | H | H | m-D14 | H | H | H | H |
| 42851 | 1 | H | H | A-34 | H | H | m-D14 | H | H | H | H |
| 42852 | 1 | H | H | A-35 | H | H | m-D14 | H | H | H | H |
| 42853 | 1 | H | H | A-36 | H | H | m-D14 | H | H | H | H |
| 42854 | 1 | H | H | A-37 | H | H | m-D14 | H | H | H | H |
| 42855 | 1 | H | H | A-38 | H | H | m-D14 | H | H | H | H |
| 42856 | 1 | H | H | A-39 | H | H | m-D14 | H | H | H | H |
| 42857 | 1 | H | H | A-40 | H | H | m-D14 | H | H | H | H |
| 42858 | 1 | H | H | A-41 | H | H | m-D14 | H | H | H | H |
| 42859 | 1 | H | H | A-42 | H | H | m-D14 | H | H | H | H |
| 42860 | 1 | H | H | A-43 | H | H | m-D14 | H | H | H | H |
| 42861 | 1 | H | H | A-44 | H | H | m-D14 | H | H | H | H |
| 42862 | 1 | H | H | A-45 | H | H | m-D14 | H | H | H | H |
| 42863 | 1 | H | H | A-46 | H | H | m-D14 | H | H | H | H |
| 42864 | 1 | H | H | A-47 | H | H | m-D14 | H | H | H | H |
| 42865 | 1 | Me | H | m-D1 | H | H | Me | H | A-1 | H | H |
| 42866 | 1 | Me | H | m-D1 | H | H | Me | H | A-2 | H | H |
| 42867 | 1 | Me | H | m-D1 | H | H | Me | H | A-3 | H | H |
| 42868 | 1 | Me | H | m-D1 | H | H | Me | H | A-4 | H | H |
| 42869 | 1 | Me | H | m-D1 | H | H | Me | H | A-5 | H | H |
| 42870 | 1 | Me | H | m-D1 | H | H | Me | H | A-6 | H | H |
| 42871 | 1 | Me | H | m-D1 | H | H | Me | H | A-7 | H | H |
| 42872 | 1 | Me | H | m-D1 | H | H | Me | H | A-8 | H | H |
| 42873 | 1 | Me | H | m-D1 | H | H | Me | H | A-9 | H | H |
| 42874 | 1 | Me | H | m-D1 | H | H | Me | H | A-10 | H | H |
| 42875 | 1 | Me | H | m-D1 | H | H | Me | H | A-11 | H | H |
| 42876 | 1 | Me | H | m-D1 | H | H | Me | H | A-12 | H | H |
| 42877 | 1 | Me | H | m-D1 | H | H | Me | H | A-13 | H | H |
| 42878 | 1 | Me | H | m-D1 | H | H | Me | H | A-14 | H | H |
| 42879 | 1 | Me | H | m-D1 | H | H | Me | H | A-15 | H | H |
| 42880 | 1 | Me | H | m-D1 | H | H | Me | H | A-16 | H | H |
| 42881 | 1 | Me | H | m-D1 | H | H | Me | H | A-17 | H | H |
| 42882 | 1 | Me | H | m-D1 | H | H | Me | H | A-18 | H | H |
| 42883 | 1 | Me | H | m-D1 | H | H | Me | H | A-19 | H | H |
| 42884 | 1 | Me | H | m-D1 | H | H | Me | H | A-20 | H | H |
| 42885 | 1 | Me | H | m-D1 | H | H | Me | H | A-21 | H | H |
| 42886 | 1 | Me | H | m-D1 | H | H | Me | H | A-22 | H | H |
| 42887 | 1 | Me | H | m-D1 | H | H | Me | H | A-23 | H | H |
| 42888 | 1 | Me | H | m-D1 | H | H | Me | H | A-24 | H | H |
| 42889 | 1 | Me | H | m-D1 | H | H | Me | H | A-25 | H | H |
| 42890 | 1 | Me | H | m-D1 | H | H | Me | H | A-26 | H | H |
| 42891 | 1 | Me | H | m-D1 | H | H | Me | H | A-27 | H | H |
| 42892 | 1 | Me | H | m-D1 | H | H | Me | H | A-28 | H | H |
| 42893 | 1 | Me | H | m-D1 | H | H | Me | H | A-29 | H | H |
| 42894 | 1 | Me | H | m-D1 | H | H | Me | H | A-30 | H | H |
| 42895 | 1 | Me | H | m-D1 | H | H | Me | H | A-31 | H | H |
| 42896 | 1 | Me | H | m-D1 | H | H | Me | H | A-32 | H | H |
| 42897 | 1 | Me | H | m-D1 | H | H | Me | H | A-33 | H | H |
| 42898 | 1 | Me | H | m-D1 | H | H | Me | H | A-34 | H | H |
| 42899 | 1 | Me | H | m-D1 | H | H | Me | H | A-35 | H | H |
| 42900 | 1 | Me | H | m-D1 | H | H | Me | H | A-36 | H | H |
| 42901 | 1 | Me | H | m-D1 | H | H | Me | H | A-37 | H | H |
| 42902 | 1 | Me | H | m-D1 | H | H | Me | H | A-38 | H | H |
| 42903 | 1 | Me | H | m-D1 | H | H | Me | H | A-39 | H | H |
| 42904 | 1 | Me | H | m-D1 | H | H | Me | H | A-40 | H | H |
| 42905 | 1 | Me | H | m-D1 | H | H | Me | H | A-41 | H | H |
| 42906 | 1 | Me | H | m-D1 | H | H | Me | H | A-42 | H | H |
| 42907 | 1 | Me | H | m-D1 | H | H | Me | H | A-43 | H | H |
| 42908 | 1 | Me | H | m-D1 | H | H | Me | H | A-44 | H | H |
| 42909 | 1 | Me | H | m-D1 | H | H | Me | H | A-45 | H | H |
| 42910 | 1 | Me | H | m-D1 | H | H | Me | H | A-46 | H | H |
| 42911 | 1 | Me | H | m-D1 | H | H | Me | H | A-47 | H | H |
| 42912 | 1 | Me | H | m-D2 | H | H | Me | H | A-1 | H | H |
| 42913 | 1 | Me | H | m-D2 | H | H | Me | H | A-2 | H | H |
| 42914 | 1 | Me | H | m-D2 | H | H | Me | H | A-3 | H | H |
| 42915 | 1 | Me | H | m-D2 | H | H | Me | H | A-4 | H | H |
| 42916 | 1 | Me | H | m-D2 | H | H | Me | H | A-5 | H | H |
| 42917 | 1 | Me | H | m-D2 | H | H | Me | H | A-6 | H | H |
| 42918 | 1 | Me | H | m-D2 | H | H | Me | H | A-7 | H | H |
| 42919 | 1 | Me | H | m-D2 | H | H | Me | H | A-8 | H | H |
| 42920 | 1 | Me | H | m-D2 | H | H | Me | H | A-9 | H | H |
| 42921 | 1 | Me | H | m-D2 | H | H | Me | H | A-10 | H | H |
| 42922 | 1 | Me | H | m-D2 | H | H | Me | H | A-11 | H | H |
| 42923 | 1 | Me | H | m-D2 | H | H | Me | H | A-12 | H | H |
| 42924 | 1 | Me | H | m-D2 | H | H | Me | H | A-13 | H | H |
| 42925 | 1 | Me | H | m-D2 | H | H | Me | H | A-14 | H | H |
| 42926 | 1 | Me | H | m-D2 | H | H | Me | H | A-15 | H | H |
| 42927 | 1 | Me | H | m-D2 | H | H | Me | H | A-16 | H | H |
| 42928 | 1 | Me | H | m-D2 | H | H | Me | H | A-17 | H | H |
| 42929 | 1 | Me | H | m-D2 | H | H | Me | H | A-18 | H | H |
| 42930 | 1 | Me | H | m-D2 | H | H | Me | H | A-19 | H | H |
| 42931 | 1 | Me | H | m-D2 | H | H | Me | H | A-20 | H | H |
| 42932 | 1 | Me | H | m-D2 | H | H | Me | H | A-21 | H | H |
| 42933 | 1 | Me | H | m-D2 | H | H | Me | H | A-22 | H | H |
| 42934 | 1 | Me | H | m-D2 | H | H | Me | H | A-23 | H | H |
| 42935 | 1 | Me | H | m-D2 | H | H | Me | H | A-24 | H | H |
| 42936 | 1 | Me | H | m-D2 | H | H | Me | H | A-25 | H | H |
| 42937 | 1 | Me | H | m-D2 | H | H | Me | H | A-26 | H | H |
| 42938 | 1 | Me | H | m-D2 | H | H | Me | H | A-27 | H | H |
| 42939 | 1 | Me | H | m-D2 | H | H | Me | H | A-28 | H | H |
| 42940 | 1 | Me | H | m-D2 | H | H | Me | H | A-29 | H | H |
| 42941 | 1 | Me | H | m-D2 | H | H | Me | H | A-30 | H | H |
| 42942 | 1 | Me | H | m-D2 | H | H | Me | H | A-31 | H | H |
| 42943 | 1 | Me | H | m-D2 | H | H | Me | H | A-32 | H | H |
| 42944 | 1 | Me | H | m-D2 | H | H | Me | H | A-33 | H | H |
| 42945 | 1 | Me | H | m-D2 | H | H | Me | H | A-34 | H | H |
| 42946 | 1 | Me | H | m-D2 | H | H | Me | H | A-35 | H | H |
| 42947 | 1 | Me | H | m-D2 | H | H | Me | H | A-36 | H | H |
| 42948 | 1 | Me | H | m-D2 | H | H | Me | H | A-37 | H | H |
| 42949 | 1 | Me | H | m-D2 | H | H | Me | H | A-38 | H | H |
| 42950 | 1 | Me | H | m-D2 | H | H | Me | H | A-39 | H | H |
| 42951 | 1 | Me | H | m-D2 | H | H | Me | H | A-40 | H | H |
| 42952 | 1 | Me | H | m-D2 | H | H | Me | H | A-41 | H | H |
| 42953 | 1 | Me | H | m-D2 | H | H | Me | H | A-42 | H | H |
| 42954 | 1 | Me | H | m-D2 | H | H | Me | H | A-43 | H | H |
| 42955 | 1 | Me | H | m-D2 | H | H | Me | H | A-44 | H | H |
| 42956 | 1 | Me | H | m-D2 | H | H | Me | H | A-45 | H | H |
| 42957 | 1 | Me | H | m-D2 | H | H | Me | H | A-46 | H | H |
| 42958 | 1 | Me | H | m-D2 | H | H | Me | H | A-47 | H | H |
| 42959 | 1 | Me | H | m-D3 | H | H | Me | H | A-1 | H | H |
| 42960 | 1 | Me | H | m-D3 | H | H | Me | H | A-2 | H | H |
| 42961 | 1 | Me | H | m-D3 | H | H | Me | H | A-3 | H | H |
| 42962 | 1 | Me | H | m-D3 | H | H | Me | H | A-4 | H | H |
| 42963 | 1 | Me | H | m-D3 | H | H | Me | H | A-5 | H | H |
| 42964 | 1 | Me | H | m-D3 | H | H | Me | H | A-6 | H | H |
| 42965 | 1 | Me | H | m-D3 | H | H | Me | H | A-7 | H | H |
| 42966 | 1 | Me | H | m-D3 | H | H | Me | H | A-8 | H | H |
| 42967 | 1 | Me | H | m-D3 | H | H | Me | H | A-9 | H | H |
| 42968 | 1 | Me | H | m-D3 | H | H | Me | H | A-10 | H | H |
| 42969 | 1 | Me | H | m-D3 | H | H | Me | H | A-11 | H | H |
| 42970 | 1 | Me | H | m-D3 | H | H | Me | H | A-12 | H | H |
| 42971 | 1 | Me | H | m-D3 | H | H | Me | H | A-13 | H | H |
| 42972 | 1 | Me | H | m-D3 | H | H | Me | H | A-14 | H | H |
| 42973 | 1 | Me | H | m-D3 | H | H | Me | H | A-15 | H | H |
| 42974 | 1 | Me | H | m-D3 | H | H | Me | H | A-16 | H | H |
| 42975 | 1 | Me | H | m-D3 | H | H | Me | H | A-17 | H | H |
| 42976 | 1 | Me | H | m-D3 | H | H | Me | H | A-18 | H | H |
| 42977 | 1 | Me | H | m-D3 | H | H | Me | H | A-19 | H | H |
| 42978 | 1 | Me | H | m-D3 | H | H | Me | H | A-20 | H | H |
| 42979 | 1 | Me | H | m-D3 | H | H | Me | H | A-21 | H | H |
| 42980 | 1 | Me | H | m-D3 | H | H | Me | H | A-22 | H | H |
| 42981 | 1 | Me | H | m-D3 | H | H | Me | H | A-23 | H | H |
| 42982 | 1 | Me | H | m-D3 | H | H | Me | H | A-24 | H | H |
| 42983 | 1 | Me | H | m-D3 | H | H | Me | H | A-25 | H | H |
| 42984 | 1 | Me | H | m-D3 | H | H | Me | H | A-26 | H | H |
| 42985 | 1 | Me | H | m-D3 | H | H | Me | H | A-27 | H | H |
| 42986 | 1 | Me | H | m-D3 | H | H | Me | H | A-28 | H | H |
| 42987 | 1 | Me | H | m-D3 | H | H | Me | H | A-29 | H | H |
| 42988 | 1 | Me | H | m-D3 | H | H | Me | H | A-30 | H | H |
| 42989 | 1 | Me | H | m-D3 | H | H | Me | H | A-31 | H | H |
| 42990 | 1 | Me | H | m-D3 | H | H | Me | H | A-32 | H | H |
| 42991 | 1 | Me | H | m-D3 | H | H | Me | H | A-33 | H | H |
| 42992 | 1 | Me | H | m-D3 | H | H | Me | H | A-34 | H | H |
| 42993 | 1 | Me | H | m-D3 | H | H | Me | H | A-35 | H | H |
| 42994 | 1 | Me | H | m-D3 | H | H | Me | H | A-36 | H | H |
| 42995 | 1 | Me | H | m-D3 | H | H | Me | H | A-37 | H | H |
| 42996 | 1 | Me | H | m-D3 | H | H | Me | H | A-38 | H | H |
| 42997 | 1 | Me | H | m-D3 | H | H | Me | H | A-39 | H | H |
| 42998 | 1 | Me | H | m-D3 | H | H | Me | H | A-40 | H | H |
| 42999 | 1 | Me | H | m-D3 | H | H | Me | H | A-41 | H | H |
| 43000 | 1 | Me | H | m-D3 | H | H | Me | H | A-42 | H | H |
| 43001 | 1 | Me | H | m-D3 | H | H | Me | H | A-43 | H | H |
| 43002 | 1 | Me | H | m-D3 | H | H | Me | H | A-44 | H | H |
| 43003 | 1 | Me | H | m-D3 | H | H | Me | H | A-45 | H | H |
| 43004 | 1 | Me | H | m-D3 | H | H | Me | H | A-46 | H | H |
| 43005 | 1 | Me | H | m-D3 | H | H | Me | H | A-47 | H | H |
| 43006 | 1 | Me | H | m-D4 | H | H | Me | H | A-1 | H | H |
| 43007 | 1 | Me | H | m-D4 | H | H | Me | H | A-2 | H | H |
| 43008 | 1 | Me | H | m-D4 | H | H | Me | H | A-3 | H | H |
| 43009 | 1 | Me | H | m-D4 | H | H | Me | H | A-4 | H | H |
| 43010 | 1 | Me | H | m-D4 | H | H | Me | H | A-5 | H | H |
| 43011 | 1 | Me | H | m-D4 | H | H | Me | H | A-6 | H | H |
| 43012 | 1 | Me | H | m-D4 | H | H | Me | H | A-7 | H | H |
| 43013 | 1 | Me | H | m-D4 | H | H | Me | H | A-8 | H | H |
| 43014 | 1 | Me | H | m-D4 | H | H | Me | H | A-9 | H | H |
| 43015 | 1 | Me | H | m-D4 | H | H | Me | H | A-10 | H | H |
| 43016 | 1 | Me | H | m-D4 | H | H | Me | H | A-11 | H | H |
| 43017 | 1 | Me | H | m-D4 | H | H | Me | H | A-12 | H | H |
| 43018 | 1 | Me | H | m-D4 | H | H | Me | H | A-13 | H | H |
| 43019 | 1 | Me | H | m-D4 | H | H | Me | H | A-14 | H | H |
| 43020 | 1 | Me | H | m-D4 | H | H | Me | H | A-15 | H | H |
| 43021 | 1 | Me | H | m-D4 | H | H | Me | H | A-16 | H | H |
| 43022 | 1 | Me | H | m-D4 | H | H | Me | H | A-17 | H | H |
| 43023 | 1 | Me | H | m-D4 | H | H | Me | H | A-18 | H | H |
| 43024 | 1 | Me | H | m-D4 | H | H | Me | H | A-19 | H | H |
| 43025 | 1 | Me | H | m-D4 | H | H | Me | H | A-20 | H | H |
| 43026 | 1 | Me | H | m-D4 | H | H | Me | H | A-21 | H | H |
| 43027 | 1 | Me | H | m-D4 | H | H | Me | H | A-22 | H | H |
| 43028 | 1 | Me | H | m-D4 | H | H | Me | H | A-23 | H | H |
| 43029 | 1 | Me | H | m-D4 | H | H | Me | H | A-24 | H | H |
| 43030 | 1 | Me | H | m-D4 | H | H | Me | H | A-25 | H | H |
| 43031 | 1 | Me | H | m-D4 | H | H | Me | H | A-26 | H | H |
| 43032 | 1 | Me | H | m-D4 | H | H | Me | H | A-27 | H | H |
| 43033 | 1 | Me | H | m-D4 | H | H | Me | H | A-28 | H | H |
| 43034 | 1 | Me | H | m-D4 | H | H | Me | H | A-29 | H | H |
| 43035 | 1 | Me | H | m-D4 | H | H | Me | H | A-30 | H | H |
| 43036 | 1 | Me | H | m-D4 | H | H | Me | H | A-31 | H | H |
| 43037 | 1 | Me | H | m-D4 | H | H | Me | H | A-32 | H | H |
| 43038 | 1 | Me | H | m-D4 | H | H | Me | H | A-33 | H | H |
| 43039 | 1 | Me | H | m-D4 | H | H | Me | H | A-34 | H | H |
| 43040 | 1 | Me | H | m-D4 | H | H | Me | H | A-35 | H | H |
| 43041 | 1 | Me | H | m-D4 | H | H | Me | H | A-36 | H | H |
| 43042 | 1 | Me | H | m-D4 | H | H | Me | H | A-37 | H | H |
| 43043 | 1 | Me | H | m-D4 | H | H | Me | H | A-38 | H | H |
| 43044 | 1 | Me | H | m-D4 | H | H | Me | H | A-39 | H | H |
| 43045 | 1 | Me | H | m-D4 | H | H | Me | H | A-40 | H | H |
| 43046 | 1 | Me | H | m-D4 | H | H | Me | H | A-41 | H | H |
| 43047 | 1 | Me | H | m-D4 | H | H | Me | H | A-42 | H | H |
| 43048 | 1 | Me | H | m-D4 | H | H | Me | H | A-43 | H | H |
| 43049 | 1 | Me | H | m-D4 | H | H | Me | H | A-44 | H | H |
| 43050 | 1 | Me | H | m-D4 | H | H | Me | H | A-45 | H | H |
| 43051 | 1 | Me | H | m-D4 | H | H | Me | H | A-46 | H | H |
| 43052 | 1 | Me | H | m-D4 | H | H | Me | H | A-47 | H | H |
| 43053 | 1 | Me | H | m-D5 | H | H | Me | H | A-1 | H | H |
| 43054 | 1 | Me | H | m-D5 | H | H | Me | H | A-2 | H | H |
| 43055 | 1 | Me | H | m-D5 | H | H | Me | H | A-3 | H | H |
| 43056 | 1 | Me | H | m-D5 | H | H | Me | H | A-4 | H | H |
| 43057 | 1 | Me | H | m-D5 | H | H | Me | H | A-5 | H | H |
| 43058 | 1 | Me | H | m-D5 | H | H | Me | H | A-6 | H | H |
| 43059 | 1 | Me | H | m-D5 | H | H | Me | H | A-7 | H | H |
| 43060 | 1 | Me | H | m-D5 | H | H | Me | H | A-8 | H | H |
| 43061 | 1 | Me | H | m-D5 | H | H | Me | H | A-9 | H | H |
| 43062 | 1 | Me | H | m-D5 | H | H | Me | H | A-10 | H | H |
| 43063 | 1 | Me | H | m-D5 | H | H | Me | H | A-11 | H | H |
| 43064 | 1 | Me | H | m-D5 | H | H | Me | H | A-12 | H | H |
| 43065 | 1 | Me | H | m-D5 | H | H | Me | H | A-13 | H | H |
| 43066 | 1 | Me | H | m-D5 | H | H | Me | H | A-14 | H | H |
| 43067 | 1 | Me | H | m-D5 | H | H | Me | H | A-15 | H | H |
| 43068 | 1 | Me | H | m-D5 | H | H | Me | H | A-16 | H | H |
| 43069 | 1 | Me | H | m-D5 | H | H | Me | H | A-17 | H | H |
| 43070 | 1 | Me | H | m-D5 | H | H | Me | H | A-18 | H | H |
| 43071 | 1 | Me | H | m-D5 | H | H | Me | H | A-19 | H | H |
| 43072 | 1 | Me | H | m-D5 | H | H | Me | H | A-20 | H | H |
| 43073 | 1 | Me | H | m-D5 | H | H | Me | H | A-21 | H | H |
| 43074 | 1 | Me | H | m-D5 | H | H | Me | H | A-22 | H | H |
| 43075 | 1 | Me | H | m-D5 | H | H | Me | H | A-23 | H | H |
| 43076 | 1 | Me | H | m-D5 | H | H | Me | H | A-24 | H | H |
| 43077 | 1 | Me | H | m-D5 | H | H | Me | H | A-25 | H | H |
| 43078 | 1 | Me | H | m-D5 | H | H | Me | H | A-26 | H | H |
| 43079 | 1 | Me | H | m-D5 | H | H | Me | H | A-27 | H | H |
| 43080 | 1 | Me | H | m-D5 | H | H | Me | H | A-28 | H | H |
| 43081 | 1 | Me | H | m-D5 | H | H | Me | H | A-29 | H | H |
| 43082 | 1 | Me | H | m-D5 | H | H | Me | H | A-30 | H | H |
| 43083 | 1 | Me | H | m-D5 | H | H | Me | H | A-31 | H | H |
| 43084 | 1 | Me | H | m-D5 | H | H | Me | H | A-32 | H | H |
| 43085 | 1 | Me | H | m-D5 | H | H | Me | H | A-33 | H | H |
| 43086 | 1 | Me | H | m-D5 | H | H | Me | H | A-34 | H | H |
| 43087 | 1 | Me | H | m-D5 | H | H | Me | H | A-35 | H | H |
| 43088 | 1 | Me | H | m-D5 | H | H | Me | H | A-36 | H | H |
| 43089 | 1 | Me | H | m-D5 | H | H | Me | H | A-37 | H | H |
| 43090 | 1 | Me | H | m-D5 | H | H | Me | H | A-38 | H | H |
| 43091 | 1 | Me | H | m-D5 | H | H | Me | H | A-39 | H | H |
| 43092 | 1 | Me | H | m-D5 | H | H | Me | H | A-40 | H | H |
| 43093 | 1 | Me | H | m-D5 | H | H | Me | H | A-41 | H | H |
| 43094 | 1 | Me | H | m-D5 | H | H | Me | H | A-42 | H | H |
| 43095 | 1 | Me | H | m-D5 | H | H | Me | H | A-43 | H | H |
| 43096 | 1 | Me | H | m-D5 | H | H | Me | H | A-44 | H | H |
| 43097 | 1 | Me | H | m-D5 | H | H | Me | H | A-45 | H | H |
| 43098 | 1 | Me | H | m-D5 | H | H | Me | H | A-46 | H | H |
| 43099 | 1 | Me | H | m-D5 | H | H | Me | H | A-47 | H | H |
| 43100 | 1 | Me | H | m-D6 | H | H | Me | H | A-1 | H | H |
| 43101 | 1 | Me | H | m-D6 | H | H | Me | H | A-2 | H | H |
| 43102 | 1 | Me | H | m-D6 | H | H | Me | H | A-3 | H | H |
| 43103 | 1 | Me | H | m-D6 | H | H | Me | H | A-4 | H | H |
| 43104 | 1 | Me | H | m-D6 | H | H | Me | H | A-5 | H | H |
| 43105 | 1 | Me | H | m-D6 | H | H | Me | H | A-6 | H | H |
| 43106 | 1 | Me | H | m-D6 | H | H | Me | H | A-7 | H | H |
| 43107 | 1 | Me | H | m-D6 | H | H | Me | H | A-8 | H | H |
| 43108 | 1 | Me | H | m-D6 | H | H | Me | H | A-9 | H | H |
| 43109 | 1 | Me | H | m-D6 | H | H | Me | H | A-10 | H | H |
| 43110 | 1 | Me | H | m-D6 | H | H | Me | H | A-11 | H | H |
| 43111 | 1 | Me | H | m-D6 | H | H | Me | H | A-12 | H | H |
| 43112 | 1 | Me | H | m-D6 | H | H | Me | H | A-13 | H | H |
| 43113 | 1 | Me | H | m-D6 | H | H | Me | H | A-14 | H | H |
| 43114 | 1 | Me | H | m-D6 | H | H | Me | H | A-15 | H | H |
| 43115 | 1 | Me | H | m-D6 | H | H | Me | H | A-16 | H | H |
| 43116 | 1 | Me | H | m-D6 | H | H | Me | H | A-17 | H | H |
| 43117 | 1 | Me | H | m-D6 | H | H | Me | H | A-18 | H | H |
| 43118 | 1 | Me | H | m-D6 | H | H | Me | H | A-19 | H | H |
| 43119 | 1 | Me | H | m-D6 | H | H | Me | H | A-20 | H | H |
| 43120 | 1 | Me | H | m-D6 | H | H | Me | H | A-21 | H | H |
| 43121 | 1 | Me | H | m-D6 | H | H | Me | H | A-22 | H | H |
| 43122 | 1 | Me | H | m-D6 | H | H | Me | H | A-23 | H | H |
| 43123 | 1 | Me | H | m-D6 | H | H | Me | H | A-24 | H | H |
| 43124 | 1 | Me | H | m-D6 | H | H | Me | H | A-25 | H | H |
| 43125 | 1 | Me | H | m-D6 | H | H | Me | H | A-26 | H | H |
| 43126 | 1 | Me | H | m-D6 | H | H | Me | H | A-27 | H | H |
| 43127 | 1 | Me | H | m-D6 | H | H | Me | H | A-28 | H | H |
| 43128 | 1 | Me | H | m-D6 | H | H | Me | H | A-29 | H | H |
| 43129 | 1 | Me | H | m-D6 | H | H | Me | H | A-30 | H | H |
| 43130 | 1 | Me | H | m-D6 | H | H | Me | H | A-31 | H | H |
| 43131 | 1 | Me | H | m-D6 | H | H | Me | H | A-32 | H | H |
| 43132 | 1 | Me | H | m-D6 | H | H | Me | H | A-33 | H | H |
| 43133 | 1 | Me | H | m-D6 | H | H | Me | H | A-34 | H | H |
| 43134 | 1 | Me | H | m-D6 | H | H | Me | H | A-35 | H | H |
| 43135 | 1 | Me | H | m-D6 | H | H | Me | H | A-36 | H | H |
| 43136 | 1 | Me | H | m-D6 | H | H | Me | H | A-37 | H | H |
| 43137 | 1 | Me | H | m-D6 | H | H | Me | H | A-38 | H | H |
| 43138 | 1 | Me | H | m-D6 | H | H | Me | H | A-39 | H | H |
| 43139 | 1 | Me | H | m-D6 | H | H | Me | H | A-40 | H | H |
| 43140 | 1 | Me | H | m-D6 | H | H | Me | H | A-41 | H | H |
| 43141 | 1 | Me | H | m-D6 | H | H | Me | H | A-42 | H | H |
| 43142 | 1 | Me | H | m-D6 | H | H | Me | H | A-43 | H | H |
| 43143 | 1 | Me | H | m-D6 | H | H | Me | H | A-44 | H | H |
| 43144 | 1 | Me | H | m-D6 | H | H | Me | H | A-45 | H | H |
| 43145 | 1 | Me | H | m-D6 | H | H | Me | H | A-46 | H | H |
| 43146 | 1 | Me | H | m-D6 | H | H | Me | H | A-47 | H | H |
| 43147 | 1 | Me | H | m-D7 | H | H | Me | H | A-1 | H | H |
| 43148 | 1 | Me | H | m-D7 | H | H | Me | H | A-2 | H | H |
| 43149 | 1 | Me | H | m-D7 | H | H | Me | H | A-3 | H | H |
| 43150 | 1 | Me | H | m-D7 | H | H | Me | H | A-4 | H | H |
| 43151 | 1 | Me | H | m-D7 | H | H | Me | H | A-5 | H | H |
| 43152 | 1 | Me | H | m-D7 | H | H | Me | H | A-6 | H | H |
| 43153 | 1 | Me | H | m-D7 | H | H | Me | H | A-7 | H | H |
| 43154 | 1 | Me | H | m-D7 | H | H | Me | H | A-8 | H | H |
| 43155 | 1 | Me | H | m-D7 | H | H | Me | H | A-9 | H | H |
| 43156 | 1 | Me | H | m-D7 | H | H | Me | H | A-10 | H | H |
| 43157 | 1 | Me | H | m-D7 | H | H | Me | H | A-11 | H | H |
| 43158 | 1 | Me | H | m-D7 | H | H | Me | H | A-12 | H | H |
| 43159 | 1 | Me | H | m-D7 | H | H | Me | H | A-13 | H | H |
| 43160 | 1 | Me | H | m-D7 | H | H | Me | H | A-14 | H | H |
| 43161 | 1 | Me | H | m-D7 | H | H | Me | H | A-15 | H | H |
| 43162 | 1 | Me | H | m-D7 | H | H | Me | H | A-16 | H | H |
| 43163 | 1 | Me | H | m-D7 | H | H | Me | H | A-17 | H | H |
| 43164 | 1 | Me | H | m-D7 | H | H | Me | H | A-18 | H | H |
| 43165 | 1 | Me | H | m-D7 | H | H | Me | H | A-19 | H | H |
| 43166 | 1 | Me | H | m-D7 | H | H | Me | H | A-20 | H | H |
| 43167 | 1 | Me | H | m-D7 | H | H | Me | H | A-21 | H | H |
| 43168 | 1 | Me | H | m-D7 | H | H | Me | H | A-22 | H | H |
| 43169 | 1 | Me | H | m-D7 | H | H | Me | H | A-23 | H | H |
| 43170 | 1 | Me | H | m-D7 | H | H | Me | H | A-24 | H | H |
| 43171 | 1 | Me | H | m-D7 | H | H | Me | H | A-25 | H | H |
| 43172 | 1 | Me | H | m-D7 | H | H | Me | H | A-26 | H | H |
| 43173 | 1 | Me | H | m-D7 | H | H | Me | H | A-27 | H | H |
| 43174 | 1 | Me | H | m-D7 | H | H | Me | H | A-28 | H | H |
| 43175 | 1 | Me | H | m-D7 | H | H | Me | H | A-29 | H | H |
| 43176 | 1 | Me | H | m-D7 | H | H | Me | H | A-30 | H | H |
| 43177 | 1 | Me | H | m-D7 | H | H | Me | H | A-31 | H | H |
| 43178 | 1 | Me | H | m-D7 | H | H | Me | H | A-32 | H | H |
| 43179 | 1 | Me | H | m-D7 | H | H | Me | H | A-33 | H | H |
| 43180 | 1 | Me | H | m-D7 | H | H | Me | H | A-34 | H | H |
| 43181 | 1 | Me | H | m-D7 | H | H | Me | H | A-35 | H | H |
| 43182 | 1 | Me | H | m-D7 | H | H | Me | H | A-36 | H | H |
| 43183 | 1 | Me | H | m-D7 | H | H | Me | H | A-37 | H | H |
| 43184 | 1 | Me | H | m-D7 | H | H | Me | H | A-38 | H | H |
| 43185 | 1 | Me | H | m-D7 | H | H | Me | H | A-39 | H | H |
| 43186 | 1 | Me | H | m-D7 | H | H | Me | H | A-40 | H | H |
| 43187 | 1 | Me | H | m-D7 | H | H | Me | H | A-41 | H | H |
| 43188 | 1 | Me | H | m-D7 | H | H | Me | H | A-42 | H | H |
| 43189 | 1 | Me | H | m-D7 | H | H | Me | H | A-43 | H | H |
| 43190 | 1 | Me | H | m-D7 | H | H | Me | H | A-44 | H | H |
| 43191 | 1 | Me | H | m-D7 | H | H | Me | H | A-45 | H | H |
| 43192 | 1 | Me | H | m-D7 | H | H | Me | H | A-46 | H | H |
| 43193 | 1 | Me | H | m-D7 | H | H | Me | H | A-47 | H | H |
| 43194 | 1 | Me | H | m-D8 | H | H | Me | H | A-1 | H | H |
| 43195 | 1 | Me | H | m-D8 | H | H | Me | H | A-2 | H | H |
| 43196 | 1 | Me | H | m-D8 | H | H | Me | H | A-3 | H | H |
| 43197 | 1 | Me | H | m-D8 | H | H | Me | H | A-4 | H | H |
| 43198 | 1 | Me | H | m-D8 | H | H | Me | H | A-5 | H | H |
| 43199 | 1 | Me | H | m-D8 | H | H | Me | H | A-6 | H | H |
| 43200 | 1 | Me | H | m-D8 | H | H | Me | H | A-7 | H | H |
| 43201 | 1 | Me | H | m-D8 | H | H | Me | H | A-8 | H | H |
| 43202 | 1 | Me | H | m-D8 | H | H | Me | H | A-9 | H | H |
| 43203 | 1 | Me | H | m-D8 | H | H | Me | H | A-10 | H | H |
| 43204 | 1 | Me | H | m-D8 | H | H | Me | H | A-11 | H | H |
| 43205 | 1 | Me | H | m-D8 | H | H | Me | H | A-12 | H | H |
| 43206 | 1 | Me | H | m-D8 | H | H | Me | H | A-13 | H | H |
| 43207 | 1 | Me | H | m-D8 | H | H | Me | H | A-14 | H | H |
| 43208 | 1 | Me | H | m-D8 | H | H | Me | H | A-15 | H | H |
| 43209 | 1 | Me | H | m-D8 | H | H | Me | H | A-16 | H | H |
| 43210 | 1 | Me | H | m-D8 | H | H | Me | H | A-17 | H | H |
| 43211 | 1 | Me | H | m-D8 | H | H | Me | H | A-18 | H | H |
| 43212 | 1 | Me | H | m-D8 | H | H | Me | H | A-19 | H | H |
| 43213 | 1 | Me | H | m-D8 | H | H | Me | H | A-20 | H | H |
| 43214 | 1 | Me | H | m-D8 | H | H | Me | H | A-21 | H | H |
| 43215 | 1 | Me | H | m-D8 | H | H | Me | H | A-22 | H | H |
| 43216 | 1 | Me | H | m-D8 | H | H | Me | H | A-23 | H | H |
| 43217 | 1 | Me | H | m-D8 | H | H | Me | H | A-24 | H | H |
| 43218 | 1 | Me | H | m-D8 | H | H | Me | H | A-25 | H | H |
| 43219 | 1 | Me | H | m-D8 | H | H | Me | H | A-26 | H | H |
| 43220 | 1 | Me | H | m-D8 | H | H | Me | H | A-27 | H | H |
| 43221 | 1 | Me | H | m-D8 | H | H | Me | H | A-28 | H | H |
| 43222 | 1 | Me | H | m-D8 | H | H | Me | H | A-29 | H | H |
| 43223 | 1 | Me | H | m-D8 | H | H | Me | H | A-30 | H | H |
| 43224 | 1 | Me | H | m-D8 | H | H | Me | H | A-31 | H | H |
| 43225 | 1 | Me | H | m-D8 | H | H | Me | H | A-32 | H | H |
| 43226 | 1 | Me | H | m-D8 | H | H | Me | H | A-33 | H | H |
| 43227 | 1 | Me | H | m-D8 | H | H | Me | H | A-34 | H | H |
| 43228 | 1 | Me | H | m-D8 | H | H | Me | H | A-35 | H | H |
| 43229 | 1 | Me | H | m-D8 | H | H | Me | H | A-36 | H | H |
| 43230 | 1 | Me | H | m-D8 | H | H | Me | H | A-37 | H | H |
| 43231 | 1 | Me | H | m-D8 | H | H | Me | H | A-38 | H | H |
| 43232 | 1 | Me | H | m-D8 | H | H | Me | H | A-39 | H | H |
| 43233 | 1 | Me | H | m-D8 | H | H | Me | H | A-40 | H | H |
| 43234 | 1 | Me | H | m-D8 | H | H | Me | H | A-41 | H | H |
| 43235 | 1 | Me | H | m-D8 | H | H | Me | H | A-42 | H | H |
| 43236 | 1 | Me | H | m-D8 | H | H | Me | H | A-43 | H | H |
| 43237 | 1 | Me | H | m-D8 | H | H | Me | H | A-44 | H | H |
| 43238 | 1 | Me | H | m-D8 | H | H | Me | H | A-45 | H | H |
| 43239 | 1 | Me | H | m-D8 | H | H | Me | H | A-46 | H | H |
| 43240 | 1 | Me | H | m-D8 | H | H | Me | H | A-47 | H | H |
| 43241 | 1 | Me | H | m-D9 | H | H | Me | H | A-1 | H | H |
| 43242 | 1 | Me | H | m-D9 | H | H | Me | H | A-2 | H | H |
| 43243 | 1 | Me | H | m-D9 | H | H | Me | H | A-3 | H | H |
| 43244 | 1 | Me | H | m-D9 | H | H | Me | H | A-4 | H | H |
| 43245 | 1 | Me | H | m-D9 | H | H | Me | H | A-5 | H | H |
| 43246 | 1 | Me | H | m-D9 | H | H | Me | H | A-6 | H | H |
| 43247 | 1 | Me | H | m-D9 | H | H | Me | H | A-7 | H | H |
| 43248 | 1 | Me | H | m-D9 | H | H | Me | H | A-8 | H | H |
| 43249 | 1 | Me | H | m-D9 | H | H | Me | H | A-9 | H | H |
| 43250 | 1 | Me | H | m-D9 | H | H | Me | H | A-10 | H | H |
| 43251 | 1 | Me | H | m-D9 | H | H | Me | H | A-11 | H | H |
| 43252 | 1 | Me | H | m-D9 | H | H | Me | H | A-12 | H | H |
| 43253 | 1 | Me | H | m-D9 | H | H | Me | H | A-13 | H | H |
| 43254 | 1 | Me | H | m-D9 | H | H | Me | H | A-14 | H | H |
| 43255 | 1 | Me | H | m-D9 | H | H | Me | H | A-15 | H | H |
| 43256 | 1 | Me | H | m-D9 | H | H | Me | H | A-16 | H | H |
| 43257 | 1 | Me | H | m-D9 | H | H | Me | H | A-17 | H | H |
| 43258 | 1 | Me | H | m-D9 | H | H | Me | H | A-18 | H | H |
| 43259 | 1 | Me | H | m-D9 | H | H | Me | H | A-19 | H | H |
| 43260 | 1 | Me | H | m-D9 | H | H | Me | H | A-20 | H | H |
| 43261 | 1 | Me | H | m-D9 | H | H | Me | H | A-21 | H | H |
| 43262 | 1 | Me | H | m-D9 | H | H | Me | H | A-22 | H | H |
| 43263 | 1 | Me | H | m-D9 | H | H | Me | H | A-23 | H | H |
| 43264 | 1 | Me | H | m-D9 | H | H | Me | H | A-24 | H | H |
| 43265 | 1 | Me | H | m-D9 | H | H | Me | H | A-25 | H | H |
| 43266 | 1 | Me | H | m-D9 | H | H | Me | H | A-26 | H | H |
| 43267 | 1 | Me | H | m-D9 | H | H | Me | H | A-27 | H | H |
| 43268 | 1 | Me | H | m-D9 | H | H | Me | H | A-28 | H | H |
| 43269 | 1 | Me | H | m-D9 | H | H | Me | H | A-29 | H | H |
| 43270 | 1 | Me | H | m-D9 | H | H | Me | H | A-30 | H | H |
| 43271 | 1 | Me | H | m-D9 | H | H | Me | H | A-31 | H | H |
| 43272 | 1 | Me | H | m-D9 | H | H | Me | H | A-32 | H | H |
| 43273 | 1 | Me | H | m-D9 | H | H | Me | H | A-33 | H | H |
| 43274 | 1 | Me | H | m-D9 | H | H | Me | H | A-34 | H | H |
| 43275 | 1 | Me | H | m-D9 | H | H | Me | H | A-35 | H | H |
| 43276 | 1 | Me | H | m-D9 | H | H | Me | H | A-36 | H | H |
| 43277 | 1 | Me | H | m-D9 | H | H | Me | H | A-37 | H | H |
| 43278 | 1 | Me | H | m-D9 | H | H | Me | H | A-38 | H | H |
| 43279 | 1 | Me | H | m-D9 | H | H | Me | H | A-39 | H | H |
| 43280 | 1 | Me | H | m-D9 | H | H | Me | H | A-40 | H | H |
| 43281 | 1 | Me | H | m-D9 | H | H | Me | H | A-41 | H | H |
| 43282 | 1 | Me | H | m-D9 | H | H | Me | H | A-42 | H | H |
| 43283 | 1 | Me | H | m-D9 | H | H | Me | H | A-43 | H | H |
| 43284 | 1 | Me | H | m-D9 | H | H | Me | H | A-44 | H | H |
| 43285 | 1 | Me | H | m-D9 | H | H | Me | H | A-45 | H | H |
| 43286 | 1 | Me | H | m-D9 | H | H | Me | H | A-46 | H | H |
| 43287 | 1 | Me | H | m-D9 | H | H | Me | H | A-47 | H | H |
| 43288 | 1 | Me | H | m-D10 | H | H | Me | H | A-1 | H | H |
| 43289 | 1 | Me | H | m-D10 | H | H | Me | H | A-2 | H | H |
| 43290 | 1 | Me | H | m-D10 | H | H | Me | H | A-3 | H | H |
| 43291 | 1 | Me | H | m-D10 | H | H | Me | H | A-4 | H | H |
| 43292 | 1 | Me | H | m-D10 | H | H | Me | H | A-5 | H | H |
| 43293 | 1 | Me | H | m-D10 | H | H | Me | H | A-6 | H | H |
| 43294 | 1 | Me | H | m-D10 | H | H | Me | H | A-7 | H | H |
| 43295 | 1 | Me | H | m-D10 | H | H | Me | H | A-8 | H | H |
| 43296 | 1 | Me | H | m-D10 | H | H | Me | H | A-9 | H | H |
| 43297 | 1 | Me | H | m-D10 | H | H | Me | H | A-10 | H | H |
| 43298 | 1 | Me | H | m-D10 | H | H | Me | H | A-11 | H | H |
| 43299 | 1 | Me | H | m-D10 | H | H | Me | H | A-12 | H | H |
| 43300 | 1 | Me | H | m-D10 | H | H | Me | H | A-13 | H | H |
| 43301 | 1 | Me | H | m-D10 | H | H | Me | H | A-14 | H | H |
| 43302 | 1 | Me | H | m-D10 | H | H | Me | H | A-15 | H | H |
| 43303 | 1 | Me | H | m-D10 | H | H | Me | H | A-16 | H | H |
| 43304 | 1 | Me | H | m-D10 | H | H | Me | H | A-17 | H | H |
| 43305 | 1 | Me | H | m-D10 | H | H | Me | H | A-18 | H | H |
| 43306 | 1 | Me | H | m-D10 | H | H | Me | H | A-19 | H | H |
| 43307 | 1 | Me | H | m-D10 | H | H | Me | H | A-20 | H | H |
| 43308 | 1 | Me | H | m-D10 | H | H | Me | H | A-21 | H | H |
| 43309 | 1 | Me | H | m-D10 | H | H | Me | H | A-22 | H | H |
| 43310 | 1 | Me | H | m-D10 | H | H | Me | H | A-23 | H | H |
| 43311 | 1 | Me | H | m-D10 | H | H | Me | H | A-24 | H | H |
| 43312 | 1 | Me | H | m-D10 | H | H | Me | H | A-25 | H | H |
| 43313 | 1 | Me | H | m-D10 | H | H | Me | H | A-26 | H | H |
| 43314 | 1 | Me | H | m-D10 | H | H | Me | H | A-27 | H | H |
| 43315 | 1 | Me | H | m-D10 | H | H | Me | H | A-28 | H | H |
| 43316 | 1 | Me | H | m-D10 | H | H | Me | H | A-29 | H | H |
| 43317 | 1 | Me | H | m-D10 | H | H | Me | H | A-30 | H | H |
| 43318 | 1 | Me | H | m-D10 | H | H | Me | H | A-31 | H | H |
| 43319 | 1 | Me | H | m-D10 | H | H | Me | H | A-32 | H | H |
| 43320 | 1 | Me | H | m-D10 | H | H | Me | H | A-33 | H | H |
| 43321 | 1 | Me | H | m-D10 | H | H | Me | H | A-34 | H | H |
| 43322 | 1 | Me | H | m-D10 | H | H | Me | H | A-35 | H | H |
| 43323 | 1 | Me | H | m-D10 | H | H | Me | H | A-36 | H | H |
| 43324 | 1 | Me | H | m-D10 | H | H | Me | H | A-37 | H | H |
| 43325 | 1 | Me | H | m-D10 | H | H | Me | H | A-38 | H | H |
| 43326 | 1 | Me | H | m-D10 | H | H | Me | H | A-39 | H | H |
| 43327 | 1 | Me | H | m-D10 | H | H | Me | H | A-40 | H | H |
| 43328 | 1 | Me | H | m-D10 | H | H | Me | H | A-41 | H | H |
| 43329 | 1 | Me | H | m-D10 | H | H | Me | H | A-42 | H | H |
| 43330 | 1 | Me | H | m-D10 | H | H | Me | H | A-43 | H | H |
| 43331 | 1 | Me | H | m-D10 | H | H | Me | H | A-44 | H | H |
| 43332 | 1 | Me | H | m-D10 | H | H | Me | H | A-45 | H | H |
| 43333 | 1 | Me | H | m-D10 | H | H | Me | H | A-46 | H | H |
| 43334 | 1 | Me | H | m-D10 | H | H | Me | H | A-47 | H | H |
| 43335 | 1 | Me | H | m-D11 | H | H | Me | H | A-1 | H | H |
| 43336 | 1 | Me | H | m-D11 | H | H | Me | H | A-2 | H | H |
| 43337 | 1 | Me | H | m-D11 | H | H | Me | H | A-3 | H | H |
| 43338 | 1 | Me | H | m-D11 | H | H | Me | H | A-4 | H | H |
| 43339 | 1 | Me | H | m-D11 | H | H | Me | H | A-5 | H | H |
| 43340 | 1 | Me | H | m-D11 | H | H | Me | H | A-6 | H | H |
| 43341 | 1 | Me | H | m-D11 | H | H | Me | H | A-7 | H | H |
| 43342 | 1 | Me | H | m-D11 | H | H | Me | H | A-8 | H | H |
| 43343 | 1 | Me | H | m-D11 | H | H | Me | H | A-9 | H | H |
| 43344 | 1 | Me | H | m-D11 | H | H | Me | H | A-10 | H | H |
| 43345 | 1 | Me | H | m-D11 | H | H | Me | H | A-11 | H | H |
| 43346 | 1 | Me | H | m-D11 | H | H | Me | H | A-12 | H | H |
| 43347 | 1 | Me | H | m-D11 | H | H | Me | H | A-13 | H | H |
| 43348 | 1 | Me | H | m-D11 | H | H | Me | H | A-14 | H | H |
| 43349 | 1 | Me | H | m-D11 | H | H | Me | H | A-15 | H | H |
| 43350 | 1 | Me | H | m-D11 | H | H | Me | H | A-16 | H | H |
| 43351 | 1 | Me | H | m-D11 | H | H | Me | H | A-17 | H | H |
| 43352 | 1 | Me | H | m-D11 | H | H | Me | H | A-18 | H | H |
| 43353 | 1 | Me | H | m-D11 | H | H | Me | H | A-19 | H | H |
| 43354 | 1 | Me | H | m-D11 | H | H | Me | H | A-20 | H | H |
| 43355 | 1 | Me | H | m-D11 | H | H | Me | H | A-21 | H | H |
| 43356 | 1 | Me | H | m-D11 | H | H | Me | H | A-22 | H | H |
| 43357 | 1 | Me | H | m-D11 | H | H | Me | H | A-23 | H | H |
| 43358 | 1 | Me | H | m-D11 | H | H | Me | H | A-24 | H | H |
| 43359 | 1 | Me | H | m-D11 | H | H | Me | H | A-25 | H | H |
| 43360 | 1 | Me | H | m-D11 | H | H | Me | H | A-26 | H | H |
| 43361 | 1 | Me | H | m-D11 | H | H | Me | H | A-27 | H | H |
| 43362 | 1 | Me | H | m-D11 | H | H | Me | H | A-28 | H | H |
| 43363 | 1 | Me | H | m-D11 | H | H | Me | H | A-29 | H | H |
| 43364 | 1 | Me | H | m-D11 | H | H | Me | H | A-30 | H | H |
| 43365 | 1 | Me | H | m-D11 | H | H | Me | H | A-31 | H | H |
| 43366 | 1 | Me | H | m-D11 | H | H | Me | H | A-32 | H | H |
| 43367 | 1 | Me | H | m-D11 | H | H | Me | H | A-33 | H | H |
| 43368 | 1 | Me | H | m-D11 | H | H | Me | H | A-34 | H | H |
| 43369 | 1 | Me | H | m-D11 | H | H | Me | H | A-35 | H | H |
| 43370 | 1 | Me | H | m-D11 | H | H | Me | H | A-36 | H | H |
| 43371 | 1 | Me | H | m-D11 | H | H | Me | H | A-37 | H | H |
| 43372 | 1 | Me | H | m-D11 | H | H | Me | H | A-38 | H | H |
| 43373 | 1 | Me | H | m-D11 | H | H | Me | H | A-39 | H | H |
| 43374 | 1 | Me | H | m-D11 | H | H | Me | H | A-40 | H | H |
| 43375 | 1 | Me | H | m-D11 | H | H | Me | H | A-41 | H | H |
| 43376 | 1 | Me | H | m-D11 | H | H | Me | H | A-42 | H | H |
| 43377 | 1 | Me | H | m-D11 | H | H | Me | H | A-43 | H | H |
| 43378 | 1 | Me | H | m-D11 | H | H | Me | H | A-44 | H | H |
| 43379 | 1 | Me | H | m-D11 | H | H | Me | H | A-45 | H | H |
| 43380 | 1 | Me | H | m-D11 | H | H | Me | H | A-46 | H | H |
| 43381 | 1 | Me | H | m-D11 | H | H | Me | H | A-47 | H | H |
| 43382 | 1 | Me | H | m-D12 | H | H | Me | H | A-1 | H | H |
| 43383 | 1 | Me | H | m-D12 | H | H | Me | H | A-2 | H | H |
| 43384 | 1 | Me | H | m-D12 | H | H | Me | H | A-3 | H | H |
| 43385 | 1 | Me | H | m-D12 | H | H | Me | H | A-4 | H | H |
| 43386 | 1 | Me | H | m-D12 | H | H | Me | H | A-5 | H | H |
| 43387 | 1 | Me | H | m-D12 | H | H | Me | H | A-6 | H | H |
| 43388 | 1 | Me | H | m-D12 | H | H | Me | H | A-7 | H | H |
| 43389 | 1 | Me | H | m-D12 | H | H | Me | H | A-8 | H | H |
| 43390 | 1 | Me | H | m-D12 | H | H | Me | H | A-9 | H | H |
| 43391 | 1 | Me | H | m-D12 | H | H | Me | H | A-10 | H | H |
| 43392 | 1 | Me | H | m-D12 | H | H | Me | H | A-11 | H | H |
| 43393 | 1 | Me | H | m-D12 | H | H | Me | H | A-12 | H | H |
| 43394 | 1 | Me | H | m-D12 | H | H | Me | H | A-13 | H | H |
| 43395 | 1 | Me | H | m-D12 | H | H | Me | H | A-14 | H | H |
| 43396 | 1 | Me | H | m-D12 | H | H | Me | H | A-15 | H | H |
| 43397 | 1 | Me | H | m-D12 | H | H | Me | H | A-16 | H | H |
| 43398 | 1 | Me | H | m-D12 | H | H | Me | H | A-17 | H | H |
| 43399 | 1 | Me | H | m-D12 | H | H | Me | H | A-18 | H | H |
| 43400 | 1 | Me | H | m-D12 | H | H | Me | H | A-19 | H | H |
| 43401 | 1 | Me | H | m-D12 | H | H | Me | H | A-20 | H | H |
| 43402 | 1 | Me | H | m-D12 | H | H | Me | H | A-21 | H | H |
| 43403 | 1 | Me | H | m-D12 | H | H | Me | H | A-22 | H | H |
| 43404 | 1 | Me | H | m-D12 | H | H | Me | H | A-23 | H | H |
| 43405 | 1 | Me | H | m-D12 | H | H | Me | H | A-24 | H | H |
| 43406 | 1 | Me | H | m-D12 | H | H | Me | H | A-25 | H | H |
| 43407 | 1 | Me | H | m-D12 | H | H | Me | H | A-26 | H | H |
| 43408 | 1 | Me | H | m-D12 | H | H | Me | H | A-27 | H | H |
| 43409 | 1 | Me | H | m-D12 | H | H | Me | H | A-28 | H | H |
| 43410 | 1 | Me | H | m-D12 | H | H | Me | H | A-29 | H | H |
| 43411 | 1 | Me | H | m-D12 | H | H | Me | H | A-30 | H | H |
| 43412 | 1 | Me | H | m-D12 | H | H | Me | H | A-31 | H | H |
| 43413 | 1 | Me | H | m-D12 | H | H | Me | H | A-32 | H | H |
| 43414 | 1 | Me | H | m-D12 | H | H | Me | H | A-33 | H | H |
| 43415 | 1 | Me | H | m-D12 | H | H | Me | H | A-34 | H | H |
| 43416 | 1 | Me | H | m-D12 | H | H | Me | H | A-35 | H | H |
| 43417 | 1 | Me | H | m-D12 | H | H | Me | H | A-36 | H | H |
| 43418 | 1 | Me | H | m-D12 | H | H | Me | H | A-37 | H | H |
| 43419 | 1 | Me | H | m-D12 | H | H | Me | H | A-38 | H | H |
| 43420 | 1 | Me | H | m-D12 | H | H | Me | H | A-39 | H | H |
| 43421 | 1 | Me | H | m-D12 | H | H | Me | H | A-40 | H | H |
| 43422 | 1 | Me | H | m-D12 | H | H | Me | H | A-41 | H | H |
| 43423 | 1 | Me | H | m-D12 | H | H | Me | H | A-42 | H | H |
| 43424 | 1 | Me | H | m-D12 | H | H | Me | H | A-43 | H | H |
| 43425 | 1 | Me | H | m-D12 | H | H | Me | H | A-44 | H | H |
| 43426 | 1 | Me | H | m-D12 | H | H | Me | H | A-45 | H | H |
| 43427 | 1 | Me | H | m-D12 | H | H | Me | H | A-46 | H | H |
| 43428 | 1 | Me | H | m-D12 | H | H | Me | H | A-47 | H | H |
| 43429 | 1 | Me | H | m-D13 | H | H | Me | H | A-1 | H | H |
| 43430 | 1 | Me | H | m-D13 | H | H | Me | H | A-2 | H | H |
| 43431 | 1 | Me | H | m-D13 | H | H | Me | H | A-3 | H | H |
| 43432 | 1 | Me | H | m-D13 | H | H | Me | H | A-4 | H | H |
| 43433 | 1 | Me | H | m-D13 | H | H | Me | H | A-5 | H | H |
| 43434 | 1 | Me | H | m-D13 | H | H | Me | H | A-6 | H | H |
| 43435 | 1 | Me | H | m-D13 | H | H | Me | H | A-7 | H | H |
| 43436 | 1 | Me | H | m-D13 | H | H | Me | H | A-8 | H | H |
| 43437 | 1 | Me | H | m-D13 | H | H | Me | H | A-9 | H | H |
| 43438 | 1 | Me | H | m-D13 | H | H | Me | H | A-10 | H | H |
| 43439 | 1 | Me | H | m-D13 | H | H | Me | H | A-11 | H | H |
| 43440 | 1 | Me | H | m-D13 | H | H | Me | H | A-12 | H | H |
| 43441 | 1 | Me | H | m-D13 | H | H | Me | H | A-13 | H | H |
| 43442 | 1 | Me | H | m-D13 | H | H | Me | H | A-14 | H | H |
| 43443 | 1 | Me | H | m-D13 | H | H | Me | H | A-15 | H | H |
| 43444 | 1 | Me | H | m-D13 | H | H | Me | H | A-16 | H | H |
| 43445 | 1 | Me | H | m-D13 | H | H | Me | H | A-17 | H | H |
| 43446 | 1 | Me | H | m-D13 | H | H | Me | H | A-18 | H | H |
| 43447 | 1 | Me | H | m-D13 | H | H | Me | H | A-19 | H | H |
| 43448 | 1 | Me | H | m-D13 | H | H | Me | H | A-20 | H | H |
| 43449 | 1 | Me | H | m-D13 | H | H | Me | H | A-21 | H | H |
| 43450 | 1 | Me | H | m-D13 | H | H | Me | H | A-22 | H | H |
| 43451 | 1 | Me | H | m-D13 | H | H | Me | H | A-23 | H | H |
| 43452 | 1 | Me | H | m-D13 | H | H | Me | H | A-24 | H | H |
| 43453 | 1 | Me | H | m-D13 | H | H | Me | H | A-25 | H | H |
| 43454 | 1 | Me | H | m-D13 | H | H | Me | H | A-26 | H | H |
| 43455 | 1 | Me | H | m-D13 | H | H | Me | H | A-27 | H | H |
| 43456 | 1 | Me | H | m-D13 | H | H | Me | H | A-28 | H | H |
| 43457 | 1 | Me | H | m-D13 | H | H | Me | H | A-29 | H | H |
| 43458 | 1 | Me | H | m-D13 | H | H | Me | H | A-30 | H | H |
| 43459 | 1 | Me | H | m-D13 | H | H | Me | H | A-31 | H | H |
| 43460 | 1 | Me | H | m-D13 | H | H | Me | H | A-32 | H | H |
| 43461 | 1 | Me | H | m-D13 | H | H | Me | H | A-33 | H | H |
| 43462 | 1 | Me | H | m-D13 | H | H | Me | H | A-34 | H | H |
| 43463 | 1 | Me | H | m-D13 | H | H | Me | H | A-35 | H | H |
| 43464 | 1 | Me | H | m-D13 | H | H | Me | H | A-36 | H | H |
| 43465 | 1 | Me | H | m-D13 | H | H | Me | H | A-37 | H | H |
| 43466 | 1 | Me | H | m-D13 | H | H | Me | H | A-38 | H | H |
| 43467 | 1 | Me | H | m-D13 | H | H | Me | H | A-39 | H | H |
| 43468 | 1 | Me | H | m-D13 | H | H | Me | H | A-40 | H | H |
| 43469 | 1 | Me | H | m-D13 | H | H | Me | H | A-41 | H | H |
| 43470 | 1 | Me | H | m-D13 | H | H | Me | H | A-42 | H | H |
| 43471 | 1 | Me | H | m-D13 | H | H | Me | H | A-43 | H | H |
| 43472 | 1 | Me | H | m-D13 | H | H | Me | H | A-44 | H | H |
| 43473 | 1 | Me | H | m-D13 | H | H | Me | H | A-45 | H | H |
| 43474 | 1 | Me | H | m-D13 | H | H | Me | H | A-46 | H | H |
| 43475 | 1 | Me | H | m-D13 | H | H | Me | H | A-47 | H | H |
| 43476 | 1 | Me | H | m-D14 | H | H | Me | H | A-1 | H | H |
| 43477 | 1 | Me | H | m-D14 | H | H | Me | H | A-2 | H | H |
| 43478 | 1 | Me | H | m-D14 | H | H | Me | H | A-3 | H | H |
| 43479 | 1 | Me | H | m-D14 | H | H | Me | H | A-4 | H | H |
| 43480 | 1 | Me | H | m-D14 | H | H | Me | H | A-5 | H | H |
| 43481 | 1 | Me | H | m-D14 | H | H | Me | H | A-6 | H | H |
| 43482 | 1 | Me | H | m-D14 | H | H | Me | H | A-7 | H | H |
| 43483 | 1 | Me | H | m-D14 | H | H | Me | H | A-8 | H | H |
| 43484 | 1 | Me | H | m-D14 | H | H | Me | H | A-9 | H | H |
| 43485 | 1 | Me | H | m-D14 | H | H | Me | H | A-10 | H | H |
| 43486 | 1 | Me | H | m-D14 | H | H | Me | H | A-11 | H | H |
| 43487 | 1 | Me | H | m-D14 | H | H | Me | H | A-12 | H | H |
| 43488 | 1 | Me | H | m-D14 | H | H | Me | H | A-13 | H | H |
| 43489 | 1 | Me | H | m-D14 | H | H | Me | H | A-14 | H | H |
| 43490 | 1 | Me | H | m-D14 | H | H | Me | H | A-15 | H | H |
| 43491 | 1 | Me | H | m-D14 | H | H | Me | H | A-16 | H | H |
| 43492 | 1 | Me | H | m-D14 | H | H | Me | H | A-17 | H | H |
| 43493 | 1 | Me | H | m-D14 | H | H | Me | H | A-18 | H | H |
| 43494 | 1 | Me | H | m-D14 | H | H | Me | H | A-19 | H | H |
| 43495 | 1 | Me | H | m-D14 | H | H | Me | H | A-20 | H | H |
| 43496 | 1 | Me | H | m-D14 | H | H | Me | H | A-21 | H | H |
| 43497 | 1 | Me | H | m-D14 | H | H | Me | H | A-22 | H | H |
| 43498 | 1 | Me | H | m-D14 | H | H | Me | H | A-23 | H | H |
| 43499 | 1 | Me | H | m-D14 | H | H | Me | H | A-24 | H | H |
| 43500 | 1 | Me | H | m-D14 | H | H | Me | H | A-25 | H | H |
| 43501 | 1 | Me | H | m-D14 | H | H | Me | H | A-26 | H | H |
| 43502 | 1 | Me | H | m-D14 | H | H | Me | H | A-27 | H | H |
| 43503 | 1 | Me | H | m-D14 | H | H | Me | H | A-28 | H | H |
| 43504 | 1 | Me | H | m-D14 | H | H | Me | H | A-29 | H | H |
| 43505 | 1 | Me | H | m-D14 | H | H | Me | H | A-30 | H | H |
| 43506 | 1 | Me | H | m-D14 | H | H | Me | H | A-31 | H | H |
| 43507 | 1 | Me | H | m-D14 | H | H | Me | H | A-32 | H | H |
| 43508 | 1 | Me | H | m-D14 | H | H | Me | H | A-33 | H | H |
| 43509 | 1 | Me | H | m-D14 | H | H | Me | H | A-34 | H | H |
| 43510 | 1 | Me | H | m-D14 | H | H | Me | H | A-35 | H | H |
| 43511 | 1 | Me | H | m-D14 | H | H | Me | H | A-36 | H | H |
| 43512 | 1 | Me | H | m-D14 | H | H | Me | H | A-37 | H | H |
| 43513 | 1 | Me | H | m-D14 | H | H | Me | H | A-38 | H | H |
| 43514 | 1 | Me | H | m-D14 | H | H | Me | H | A-39 | H | H |
| 43515 | 1 | Me | H | m-D14 | H | H | Me | H | A-40 | H | H |
| 43516 | 1 | Me | H | m-D14 | H | H | Me | H | A-41 | H | H |
| 43517 | 1 | Me | H | m-D14 | H | H | Me | H | A-42 | H | H |
| 43518 | 1 | Me | H | m-D14 | H | H | Me | H | A-43 | H | H |
| 43519 | 1 | Me | H | m-D14 | H | H | Me | H | A-44 | H | H |
| 43520 | 1 | Me | H | m-D14 | H | H | Me | H | A-45 | H | H |
| 43521 | 1 | Me | H | m-D14 | H | H | Me | H | A-46 | H | H |
| 43522 | 1 | Me | H | m-D14 | H | H | Me | H | A-47 | H | H |
| 43523 | 1 | tBu | H | m-D1 | H | H | tBu | H | A-1 | H | H |
| 43524 | 1 | tBu | H | m-D1 | H | H | tBu | H | A-2 | H | H |
| 43525 | 1 | tBu | H | m-D1 | H | H | tBu | H | A-3 | H | H |
| 43526 | 1 | tBu | H | m-D1 | H | H | tBu | H | A-4 | H | H |
| 43527 | 1 | tBu | H | m-D1 | H | H | tBu | H | A-5 | H | H |
| 43528 | 1 | tBu | H | m-D1 | H | H | tBu | H | A-6 | H | H |
| 43529 | 1 | tBu | H | m-D1 | H | H | tBu | H | A-7 | H | H |
| 43530 | 1 | tBu | H | m-D1 | H | H | tBu | H | A-8 | H | H |
| 43531 | 1 | tBu | H | m-D1 | H | H | tBu | H | A-9 | H | H |
| 43532 | 1 | tBu | H | m-D1 | H | H | tBu | H | A-10 | H | H |
| 43533 | 1 | tBu | H | m-D1 | H | H | tBu | H | A-11 | H | H |
| 43534 | 1 | tBu | H | m-D1 | H | H | tBu | H | A-12 | H | H |
| 43535 | 1 | tBu | H | m-D1 | H | H | tBu | H | A-13 | H | H |
| 43536 | 1 | tBu | H | m-D1 | H | H | tBu | H | A-14 | H | H |
| 43537 | 1 | tBu | H | m-D1 | H | H | tBu | H | A-15 | H | H |
| 43538 | 1 | tBu | H | m-D1 | H | H | tBu | H | A-16 | H | H |
| 43539 | 1 | tBu | H | m-D1 | H | H | tBu | H | A-17 | H | H |
| 43540 | 1 | tBu | H | m-D1 | H | H | tBu | H | A-18 | H | H |
| 43541 | 1 | tBu | H | m-D1 | H | H | tBu | H | A-19 | H | H |
| 43542 | 1 | tBu | H | m-D1 | H | H | tBu | H | A-20 | H | H |
| 43543 | 1 | tBu | H | m-D1 | H | H | tBu | H | A-21 | H | H |
| 43544 | 1 | tBu | H | m-D1 | H | H | tBu | H | A-22 | H | H |
| 43545 | 1 | tBu | H | m-D1 | H | H | tBu | H | A-23 | H | H |
| 43546 | 1 | tBu | H | m-D1 | H | H | tBu | H | A-24 | H | H |
| 43547 | 1 | tBu | H | m-D1 | H | H | tBu | H | A-25 | H | H |
| 43548 | 1 | tBu | H | m-D1 | H | H | tBu | H | A-26 | H | H |
| 43549 | 1 | tBu | H | m-D1 | H | H | tBu | H | A-27 | H | H |
| 43550 | 1 | tBu | H | m-D1 | H | H | tBu | H | A-28 | H | H |
| 43551 | 1 | tBu | H | m-D1 | H | H | tBu | H | A-29 | H | H |
| 43552 | 1 | tBu | H | m-D1 | H | H | tBu | H | A-30 | H | H |
| 43553 | 1 | tBu | H | m-D1 | H | H | tBu | H | A-31 | H | H |
| 43554 | 1 | tBu | H | m-D1 | H | H | tBu | H | A-32 | H | H |
| 43555 | 1 | tBu | H | m-D1 | H | H | tBu | H | A-33 | H | H |
| 43556 | 1 | tBu | H | m-D1 | H | H | tBu | H | A-34 | H | H |
| 43557 | 1 | tBu | H | m-D1 | H | H | tBu | H | A-35 | H | H |
| 43558 | 1 | tBu | H | m-D1 | H | H | tBu | H | A-36 | H | H |
| 43559 | 1 | tBu | H | m-D1 | H | H | tBu | H | A-37 | H | H |
| 43560 | 1 | tBu | H | m-D1 | H | H | tBu | H | A-38 | H | H |
| 43561 | 1 | tBu | H | m-D1 | H | H | tBu | H | A-39 | H | H |
| 43562 | 1 | tBu | H | m-D1 | H | H | tBu | H | A-40 | H | H |
| 43563 | 1 | tBu | H | m-D1 | H | H | tBu | H | A-41 | H | H |
| 43564 | 1 | tBu | H | m-D1 | H | H | tBu | H | A-42 | H | H |
| 43565 | 1 | tBu | H | m-D1 | H | H | tBu | H | A-43 | H | H |
| 43566 | 1 | tBu | H | m-D1 | H | H | tBu | H | A-44 | H | H |
| 43567 | 1 | tBu | H | m-D1 | H | H | tBu | H | A-45 | H | H |
| 43568 | 1 | tBu | H | m-D1 | H | H | tBu | H | A-46 | H | H |
| 43569 | 1 | tBu | H | m-D1 | H | H | tBu | H | A-47 | H | H |
| 43570 | 1 | tBu | H | m-D2 | H | H | tBu | H | A-1 | H | H |
| 43571 | 1 | tBu | H | m-D2 | H | H | tBu | H | A-2 | H | H |
| 43572 | 1 | tBu | H | m-D2 | H | H | tBu | H | A-3 | H | H |
| 43573 | 1 | tBu | H | m-D2 | H | H | tBu | H | A-4 | H | H |
| 43574 | 1 | tBu | H | m-D2 | H | H | tBu | H | A-5 | H | H |
| 43575 | 1 | tBu | H | m-D2 | H | H | tBu | H | A-6 | H | H |
| 43576 | 1 | tBu | H | m-D2 | H | H | tBu | H | A-7 | H | H |
| 43577 | 1 | tBu | H | m-D2 | H | H | tBu | H | A-8 | H | H |
| 43578 | 1 | tBu | H | m-D2 | H | H | tBu | H | A-9 | H | H |
| 43579 | 1 | tBu | H | m-D2 | H | H | tBu | H | A-10 | H | H |
| 43580 | 1 | tBu | H | m-D2 | H | H | tBu | H | A-11 | H | H |
| 43581 | 1 | tBu | H | m-D2 | H | H | tBu | H | A-12 | H | H |
| 43582 | 1 | tBu | H | m-D2 | H | H | tBu | H | A-13 | H | H |
| 43583 | 1 | tBu | H | m-D2 | H | H | tBu | H | A-14 | H | H |
| 43584 | 1 | tBu | H | m-D2 | H | H | tBu | H | A-15 | H | H |
| 43585 | 1 | tBu | H | m-D2 | H | H | tBu | H | A-16 | H | H |
| 43586 | 1 | tBu | H | m-D2 | H | H | tBu | H | A-17 | H | H |
| 43587 | 1 | tBu | H | m-D2 | H | H | tBu | H | A-18 | H | H |
| 43588 | 1 | tBu | H | m-D2 | H | H | tBu | H | A-19 | H | H |
| 43589 | 1 | tBu | H | m-D2 | H | H | tBu | H | A-20 | H | H |
| 43590 | 1 | tBu | H | m-D2 | H | H | tBu | H | A-21 | H | H |
| 43591 | 1 | tBu | H | m-D2 | H | H | tBu | H | A-22 | H | H |
| 43592 | 1 | tBu | H | m-D2 | H | H | tBu | H | A-23 | H | H |
| 43593 | 1 | tBu | H | m-D2 | H | H | tBu | H | A-24 | H | H |
| 43594 | 1 | tBu | H | m-D2 | H | H | tBu | H | A-25 | H | H |
| 43595 | 1 | tBu | H | m-D2 | H | H | tBu | H | A-26 | H | H |
| 43596 | 1 | tBu | H | m-D2 | H | H | tBu | H | A-27 | H | H |
| 43597 | 1 | tBu | H | m-D2 | H | H | tBu | H | A-28 | H | H |
| 43598 | 1 | tBu | H | m-D2 | H | H | tBu | H | A-29 | H | H |
| 43599 | 1 | tBu | H | m-D2 | H | H | tBu | H | A-30 | H | H |
| 43600 | 1 | tBu | H | m-D2 | H | H | tBu | H | A-31 | H | H |
| 43601 | 1 | tBu | H | m-D2 | H | H | tBu | H | A-32 | H | H |
| 43602 | 1 | tBu | H | m-D2 | H | H | tBu | H | A-33 | H | H |
| 43603 | 1 | tBu | H | m-D2 | H | H | tBu | H | A-34 | H | H |
| 43604 | 1 | tBu | H | m-D2 | H | H | tBu | H | A-35 | H | H |
| 43605 | 1 | tBu | H | m-D2 | H | H | tBu | H | A-36 | H | H |
| 43606 | 1 | tBu | H | m-D2 | H | H | tBu | H | A-37 | H | H |
| 43607 | 1 | tBu | H | m-D2 | H | H | tBu | H | A-38 | H | H |
| 43608 | 1 | tBu | H | m-D2 | H | H | tBu | H | A-39 | H | H |
| 43609 | 1 | tBu | H | m-D2 | H | H | tBu | H | A-40 | H | H |
| 43610 | 1 | tBu | H | m-D2 | H | H | tBu | H | A-41 | H | H |
| 43611 | 1 | tBu | H | m-D2 | H | H | tBu | H | A-42 | H | H |
| 43612 | 1 | tBu | H | m-D2 | H | H | tBu | H | A-43 | H | H |
| 43613 | 1 | tBu | H | m-D2 | H | H | tBu | H | A-44 | H | H |
| 43614 | 1 | tBu | H | m-D2 | H | H | tBu | H | A-45 | H | H |
| 43615 | 1 | tBu | H | m-D2 | H | H | tBu | H | A-46 | H | H |
| 43616 | 1 | tBu | H | m-D2 | H | H | tBu | H | A-47 | H | H |
| 43617 | 1 | tBu | H | m-D3 | H | H | tBu | H | A-1 | H | H |
| 43618 | 1 | tBu | H | m-D3 | H | H | tBu | H | A-2 | H | H |
| 43619 | 1 | tBu | H | m-D3 | H | H | tBu | H | A-3 | H | H |
| 43620 | 1 | tBu | H | m-D3 | H | H | tBu | H | A-4 | H | H |
| 43621 | 1 | tBu | H | m-D3 | H | H | tBu | H | A-5 | H | H |
| 43622 | 1 | tBu | H | m-D3 | H | H | tBu | H | A-6 | H | H |
| 43623 | 1 | tBu | H | m-D3 | H | H | tBu | H | A-7 | H | H |
| 43624 | 1 | tBu | H | m-D3 | H | H | tBu | H | A-8 | H | H |
| 43625 | 1 | tBu | H | m-D3 | H | H | tBu | H | A-9 | H | H |
| 43626 | 1 | tBu | H | m-D3 | H | H | tBu | H | A-10 | H | H |
| 43627 | 1 | tBu | H | m-D3 | H | H | tBu | H | A-11 | H | H |
| 43628 | 1 | tBu | H | m-D3 | H | H | tBu | H | A-12 | H | H |
| 43629 | 1 | tBu | H | m-D3 | H | H | tBu | H | A-13 | H | H |
| 43630 | 1 | tBu | H | m-D3 | H | H | tBu | H | A-14 | H | H |
| 43631 | 1 | tBu | H | m-D3 | H | H | tBu | H | A-15 | H | H |
| 43632 | 1 | tBu | H | m-D3 | H | H | tBu | H | A-16 | H | H |
| 43633 | 1 | tBu | H | m-D3 | H | H | tBu | H | A-17 | H | H |
| 43634 | 1 | tBu | H | m-D3 | H | H | tBu | H | A-18 | H | H |
| 43635 | 1 | tBu | H | m-D3 | H | H | tBu | H | A-19 | H | H |
| 43636 | 1 | tBu | H | m-D3 | H | H | tBu | H | A-20 | H | H |
| 43637 | 1 | tBu | H | m-D3 | H | H | tBu | H | A-21 | H | H |
| 43638 | 1 | tBu | H | m-D3 | H | H | tBu | H | A-22 | H | H |
| 43639 | 1 | tBu | H | m-D3 | H | H | tBu | H | A-23 | H | H |
| 43640 | 1 | tBu | H | m-D3 | H | H | tBu | H | A-24 | H | H |
| 43641 | 1 | tBu | H | m-D3 | H | H | tBu | H | A-25 | H | H |
| 43642 | 1 | tBu | H | m-D3 | H | H | tBu | H | A-26 | H | H |
| 43643 | 1 | tBu | H | m-D3 | H | H | tBu | H | A-27 | H | H |
| 43644 | 1 | tBu | H | m-D3 | H | H | tBu | H | A-28 | H | H |
| 43645 | 1 | tBu | H | m-D3 | H | H | tBu | H | A-29 | H | H |
| 43646 | 1 | tBu | H | m-D3 | H | H | tBu | H | A-30 | H | H |
| 43647 | 1 | tBu | H | m-D3 | H | H | tBu | H | A-31 | H | H |
| 43648 | 1 | tBu | H | m-D3 | H | H | tBu | H | A-32 | H | H |
| 43649 | 1 | tBu | H | m-D3 | H | H | tBu | H | A-33 | H | H |
| 43650 | 1 | tBu | H | m-D3 | H | H | tBu | H | A-34 | H | H |
| 43651 | 1 | tBu | H | m-D3 | H | H | tBu | H | A-35 | H | H |
| 43652 | 1 | tBu | H | m-D3 | H | H | tBu | H | A-36 | H | H |
| 43653 | 1 | tBu | H | m-D3 | H | H | tBu | H | A-37 | H | H |
| 43654 | 1 | tBu | H | m-D3 | H | H | tBu | H | A-38 | H | H |
| 43655 | 1 | tBu | H | m-D3 | H | H | tBu | H | A-39 | H | H |
| 43656 | 1 | tBu | H | m-D3 | H | H | tBu | H | A-40 | H | H |
| 43657 | 1 | tBu | H | m-D3 | H | H | tBu | H | A-41 | H | H |
| 43658 | 1 | tBu | H | m-D3 | H | H | tBu | H | A-42 | H | H |
| 43659 | 1 | tBu | H | m-D3 | H | H | tBu | H | A-43 | H | H |
| 43660 | 1 | tBu | H | m-D3 | H | H | tBu | H | A-44 | H | H |
| 43661 | 1 | tBu | H | m-D3 | H | H | tBu | H | A-45 | H | H |
| 43662 | 1 | tBu | H | m-D3 | H | H | tBu | H | A-46 | H | H |
| 43663 | 1 | tBu | H | m-D3 | H | H | tBu | H | A-47 | H | H |
| 43664 | 1 | tBu | H | m-D4 | H | H | tBu | H | A-1 | H | H |
| 43665 | 1 | tBu | H | m-D4 | H | H | tBu | H | A-2 | H | H |
| 43666 | 1 | tBu | H | m-D4 | H | H | tBu | H | A-3 | H | H |
| 43667 | 1 | tBu | H | m-D4 | H | H | tBu | H | A-4 | H | H |
| 43668 | 1 | tBu | H | m-D4 | H | H | tBu | H | A-5 | H | H |
| 43669 | 1 | tBu | H | m-D4 | H | H | tBu | H | A-6 | H | H |
| 43670 | 1 | tBu | H | m-D4 | H | H | tBu | H | A-7 | H | H |
| 43671 | 1 | tBu | H | m-D4 | H | H | tBu | H | A-8 | H | H |
| 43672 | 1 | tBu | H | m-D4 | H | H | tBu | H | A-9 | H | H |
| 43673 | 1 | tBu | H | m-D4 | H | H | tBu | H | A-10 | H | H |
| 43674 | 1 | tBu | H | m-D4 | H | H | tBu | H | A-11 | H | H |
| 43675 | 1 | tBu | H | m-D4 | H | H | tBu | H | A-12 | H | H |
| 43676 | 1 | tBu | H | m-D4 | H | H | tBu | H | A-13 | H | H |
| 43677 | 1 | tBu | H | m-D4 | H | H | tBu | H | A-14 | H | H |
| 43678 | 1 | tBu | H | m-D4 | H | H | tBu | H | A-15 | H | H |
| 43679 | 1 | tBu | H | m-D4 | H | H | tBu | H | A-16 | H | H |
| 43680 | 1 | tBu | H | m-D4 | H | H | tBu | H | A-17 | H | H |
| 43681 | 1 | tBu | H | m-D4 | H | H | tBu | H | A-18 | H | H |
| 43682 | 1 | tBu | H | m-D4 | H | H | tBu | H | A-19 | H | H |
| 43683 | 1 | tBu | H | m-D4 | H | H | tBu | H | A-20 | H | H |
| 43684 | 1 | tBu | H | m-D4 | H | H | tBu | H | A-21 | H | H |
| 43685 | 1 | tBu | H | m-D4 | H | H | tBu | H | A-22 | H | H |
| 43686 | 1 | tBu | H | m-D4 | H | H | tBu | H | A-23 | H | H |
| 43687 | 1 | tBu | H | m-D4 | H | H | tBu | H | A-24 | H | H |
| 43688 | 1 | tBu | H | m-D4 | H | H | tBu | H | A-25 | H | H |
| 43689 | 1 | tBu | H | m-D4 | H | H | tBu | H | A-26 | H | H |
| 43690 | 1 | tBu | H | m-D4 | H | H | tBu | H | A-27 | H | H |
| 43691 | 1 | tBu | H | m-D4 | H | H | tBu | H | A-28 | H | H |
| 43692 | 1 | tBu | H | m-D4 | H | H | tBu | H | A-29 | H | H |
| 43693 | 1 | tBu | H | m-D4 | H | H | tBu | H | A-30 | H | H |
| 43694 | 1 | tBu | H | m-D4 | H | H | tBu | H | A-31 | H | H |
| 43695 | 1 | tBu | H | m-D4 | H | H | tBu | H | A-32 | H | H |
| 43696 | 1 | tBu | H | m-D4 | H | H | tBu | H | A-33 | H | H |
| 43697 | 1 | tBu | H | m-D4 | H | H | tBu | H | A-34 | H | H |
| 43698 | 1 | tBu | H | m-D4 | H | H | tBu | H | A-35 | H | H |
| 43699 | 1 | tBu | H | m-D4 | H | H | tBu | H | A-36 | H | H |
| 43700 | 1 | tBu | H | m-D4 | H | H | tBu | H | A-37 | H | H |
| 43701 | 1 | tBu | H | m-D4 | H | H | tBu | H | A-38 | H | H |
| 43702 | 1 | tBu | H | m-D4 | H | H | tBu | H | A-39 | H | H |
| 43703 | 1 | tBu | H | m-D4 | H | H | tBu | H | A-40 | H | H |
| 43704 | 1 | tBu | H | m-D4 | H | H | tBu | H | A-41 | H | H |
| 43705 | 1 | tBu | H | m-D4 | H | H | tBu | H | A-42 | H | H |
| 43706 | 1 | tBu | H | m-D4 | H | H | tBu | H | A-43 | H | H |
| 43707 | 1 | tBu | H | m-D4 | H | H | tBu | H | A-44 | H | H |
| 43708 | 1 | tBu | H | m-D4 | H | H | tBu | H | A-45 | H | H |
| 43709 | 1 | tBu | H | m-D4 | H | H | tBu | H | A-46 | H | H |
| 43710 | 1 | tBu | H | m-D4 | H | H | tBu | H | A-47 | H | H |
| 43711 | 1 | tBu | H | m-D5 | H | H | tBu | H | A-1 | H | H |
| 43712 | 1 | tBu | H | m-D5 | H | H | tBu | H | A-2 | H | H |
| 43713 | 1 | tBu | H | m-D5 | H | H | tBu | H | A-3 | H | H |
| 43714 | 1 | tBu | H | m-D5 | H | H | tBu | H | A-4 | H | H |
| 43715 | 1 | tBu | H | m-D5 | H | H | tBu | H | A-5 | H | H |
| 43716 | 1 | tBu | H | m-D5 | H | H | tBu | H | A-6 | H | H |
| 43717 | 1 | tBu | H | m-D5 | H | H | tBu | H | A-7 | H | H |
| 43718 | 1 | tBu | H | m-D5 | H | H | tBu | H | A-8 | H | H |
| 43719 | 1 | tBu | H | m-D5 | H | H | tBu | H | A-9 | H | H |
| 43720 | 1 | tBu | H | m-D5 | H | H | tBu | H | A-10 | H | H |
| 43721 | 1 | tBu | H | m-D5 | H | H | tBu | H | A-11 | H | H |
| 43722 | 1 | tBu | H | m-D5 | H | H | tBu | H | A-12 | H | H |
| 43723 | 1 | tBu | H | m-D5 | H | H | tBu | H | A-13 | H | H |
| 43724 | 1 | tBu | H | m-D5 | H | H | tBu | H | A-14 | H | H |
| 43725 | 1 | tBu | H | m-D5 | H | H | tBu | H | A-15 | H | H |
| 43726 | 1 | tBu | H | m-D5 | H | H | tBu | H | A-16 | H | H |
| 43727 | 1 | tBu | H | m-D5 | H | H | tBu | H | A-17 | H | H |
| 43728 | 1 | tBu | H | m-D5 | H | H | tBu | H | A-18 | H | H |
| 43729 | 1 | tBu | H | m-D5 | H | H | tBu | H | A-19 | H | H |
| 43730 | 1 | tBu | H | m-D5 | H | H | tBu | H | A-20 | H | H |
| 43731 | 1 | tBu | H | m-D5 | H | H | tBu | H | A-21 | H | H |
| 43732 | 1 | tBu | H | m-D5 | H | H | tBu | H | A-22 | H | H |
| 43733 | 1 | tBu | H | m-D5 | H | H | tBu | H | A-23 | H | H |
| 43734 | 1 | tBu | H | m-D5 | H | H | tBu | H | A-24 | H | H |
| 43735 | 1 | tBu | H | m-D5 | H | H | tBu | H | A-25 | H | H |
| 43736 | 1 | tBu | H | m-D5 | H | H | tBu | H | A-26 | H | H |
| 43737 | 1 | tBu | H | m-D5 | H | H | tBu | H | A-27 | H | H |
| 43738 | 1 | tBu | H | m-D5 | H | H | tBu | H | A-28 | H | H |
| 43739 | 1 | tBu | H | m-D5 | H | H | tBu | H | A-29 | H | H |
| 43740 | 1 | tBu | H | m-D5 | H | H | tBu | H | A-30 | H | H |
| 43741 | 1 | tBu | H | m-D5 | H | H | tBu | H | A-31 | H | H |
| 43742 | 1 | tBu | H | m-D5 | H | H | tBu | H | A-32 | H | H |
| 43743 | 1 | tBu | H | m-D5 | H | H | tBu | H | A-33 | H | H |
| 43744 | 1 | tBu | H | m-D5 | H | H | tBu | H | A-34 | H | H |
| 43745 | 1 | tBu | H | m-D5 | H | H | tBu | H | A-35 | H | H |
| 43746 | 1 | tBu | H | m-D5 | H | H | tBu | H | A-36 | H | H |
| 43747 | 1 | tBu | H | m-D5 | H | H | tBu | H | A-37 | H | H |
| 43748 | 1 | tBu | H | m-D5 | H | H | tBu | H | A-38 | H | H |
| 43749 | 1 | tBu | H | m-D5 | H | H | tBu | H | A-39 | H | H |
| 43750 | 1 | tBu | H | m-D5 | H | H | tBu | H | A-40 | H | H |
| 43751 | 1 | tBu | H | m-D5 | H | H | tBu | H | A-41 | H | H |
| 43752 | 1 | tBu | H | m-D5 | H | H | tBu | H | A-42 | H | H |
| 43753 | 1 | tBu | H | m-D5 | H | H | tBu | H | A-43 | H | H |
| 43754 | 1 | tBu | H | m-D5 | H | H | tBu | H | A-44 | H | H |
| 43755 | 1 | tBu | H | m-D5 | H | H | tBu | H | A-45 | H | H |
| 43756 | 1 | tBu | H | m-D5 | H | H | tBu | H | A-46 | H | H |
| 43757 | 1 | tBu | H | m-D5 | H | H | tBu | H | A-47 | H | H |
| 43758 | 1 | tBu | H | m-D6 | H | H | tBu | H | A-1 | H | H |
| 43759 | 1 | tBu | H | m-D6 | H | H | tBu | H | A-2 | H | H |
| 43760 | 1 | tBu | H | m-D6 | H | H | tBu | H | A-3 | H | H |
| 43761 | 1 | tBu | H | m-D6 | H | H | tBu | H | A-4 | H | H |
| 43762 | 1 | tBu | H | m-D6 | H | H | tBu | H | A-5 | H | H |
| 43763 | 1 | tBu | H | m-D6 | H | H | tBu | H | A-6 | H | H |
| 43764 | 1 | tBu | H | m-D6 | H | H | tBu | H | A-7 | H | H |
| 43765 | 1 | tBu | H | m-D6 | H | H | tBu | H | A-8 | H | H |
| 43766 | 1 | tBu | H | m-D6 | H | H | tBu | H | A-9 | H | H |
| 43767 | 1 | tBu | H | m-D6 | H | H | tBu | H | A-10 | H | H |
| 43768 | 1 | tBu | H | m-D6 | H | H | tBu | H | A-11 | H | H |
| 43769 | 1 | tBu | H | m-D6 | H | H | tBu | H | A-12 | H | H |
| 43770 | 1 | tBu | H | m-D6 | H | H | tBu | H | A-13 | H | H |
| 43771 | 1 | tBu | H | m-D6 | H | H | tBu | H | A-14 | H | H |
| 43772 | 1 | tBu | H | m-D6 | H | H | tBu | H | A-15 | H | H |
| 43773 | 1 | tBu | H | m-D6 | H | H | tBu | H | A-16 | H | H |
| 43774 | 1 | tBu | H | m-D6 | H | H | tBu | H | A-17 | H | H |
| 43775 | 1 | tBu | H | m-D6 | H | H | tBu | H | A-18 | H | H |
| 43776 | 1 | tBu | H | m-D6 | H | H | tBu | H | A-19 | H | H |
| 43777 | 1 | tBu | H | m-D6 | H | H | tBu | H | A-20 | H | H |
| 43778 | 1 | tBu | H | m-D6 | H | H | tBu | H | A-21 | H | H |
| 43779 | 1 | tBu | H | m-D6 | H | H | tBu | H | A-22 | H | H |
| 43780 | 1 | tBu | H | m-D6 | H | H | tBu | H | A-23 | H | H |
| 43781 | 1 | tBu | H | m-D6 | H | H | tBu | H | A-24 | H | H |
| 43782 | 1 | tBu | H | m-D6 | H | H | tBu | H | A-25 | H | H |
| 43783 | 1 | tBu | H | m-D6 | H | H | tBu | H | A-26 | H | H |
| 43784 | 1 | tBu | H | m-D6 | H | H | tBu | H | A-27 | H | H |
| 43785 | 1 | tBu | H | m-D6 | H | H | tBu | H | A-28 | H | H |
| 43786 | 1 | tBu | H | m-D6 | H | H | tBu | H | A-29 | H | H |
| 43787 | 1 | tBu | H | m-D6 | H | H | tBu | H | A-30 | H | H |
| 43788 | 1 | tBu | H | m-D6 | H | H | tBu | H | A-31 | H | H |
| 43789 | 1 | tBu | H | m-D6 | H | H | tBu | H | A-32 | H | H |
| 43790 | 1 | tBu | H | m-D6 | H | H | tBu | H | A-33 | H | H |
| 43791 | 1 | tBu | H | m-D6 | H | H | tBu | H | A-34 | H | H |
| 43792 | 1 | tBu | H | m-D6 | H | H | tBu | H | A-35 | H | H |
| 43793 | 1 | tBu | H | m-D6 | H | H | tBu | H | A-36 | H | H |
| 43794 | 1 | tBu | H | m-D6 | H | H | tBu | H | A-37 | H | H |
| 43795 | 1 | tBu | H | m-D6 | H | H | tBu | H | A-38 | H | H |
| 43796 | 1 | tBu | H | m-D6 | H | H | tBu | H | A-39 | H | H |
| 43797 | 1 | tBu | H | m-D6 | H | H | tBu | H | A-40 | H | H |
| 43798 | 1 | tBu | H | m-D6 | H | H | tBu | H | A-41 | H | H |
| 43799 | 1 | tBu | H | m-D6 | H | H | tBu | H | A-42 | H | H |
| 43800 | 1 | tBu | H | m-D6 | H | H | tBu | H | A-43 | H | H |
| 43801 | 1 | tBu | H | m-D6 | H | H | tBu | H | A-44 | H | H |
| 43802 | 1 | tBu | H | m-D6 | H | H | tBu | H | A-45 | H | H |
| 43803 | 1 | tBu | H | m-D6 | H | H | tBu | H | A-46 | H | H |
| 43804 | 1 | tBu | H | m-D6 | H | H | tBu | H | A-47 | H | H |
| 43805 | 1 | tBu | H | m-D7 | H | H | tBu | H | A-1 | H | H |
| 43806 | 1 | tBu | H | m-D7 | H | H | tBu | H | A-2 | H | H |
| 43807 | 1 | tBu | H | m-D7 | H | H | tBu | H | A-3 | H | H |
| 43808 | 1 | tBu | H | m-D7 | H | H | tBu | H | A-4 | H | H |
| 43809 | 1 | tBu | H | m-D7 | H | H | tBu | H | A-5 | H | H |
| 43810 | 1 | tBu | H | m-D7 | H | H | tBu | H | A-6 | H | H |
| 43811 | 1 | tBu | H | m-D7 | H | H | tBu | H | A-7 | H | H |
| 43812 | 1 | tBu | H | m-D7 | H | H | tBu | H | A-8 | H | H |
| 43813 | 1 | tBu | H | m-D7 | H | H | tBu | H | A-9 | H | H |
| 43814 | 1 | tBu | H | m-D7 | H | H | tBu | H | A-10 | H | H |
| 43815 | 1 | tBu | H | m-D7 | H | H | tBu | H | A-11 | H | H |
| 43816 | 1 | tBu | H | m-D7 | H | H | tBu | H | A-12 | H | H |
| 43817 | 1 | tBu | H | m-D7 | H | H | tBu | H | A-13 | H | H |
| 43818 | 1 | tBu | H | m-D7 | H | H | tBu | H | A-14 | H | H |
| 43819 | 1 | tBu | H | m-D7 | H | H | tBu | H | A-15 | H | H |
| 43820 | 1 | tBu | H | m-D7 | H | H | tBu | H | A-16 | H | H |
| 43821 | 1 | tBu | H | m-D7 | H | H | tBu | H | A-17 | H | H |
| 43822 | 1 | tBu | H | m-D7 | H | H | tBu | H | A-18 | H | H |
| 43823 | 1 | tBu | H | m-D7 | H | H | tBu | H | A-19 | H | H |
| 43824 | 1 | tBu | H | m-D7 | H | H | tBu | H | A-20 | H | H |
| 43825 | 1 | tBu | H | m-D7 | H | H | tBu | H | A-21 | H | H |
| 43826 | 1 | tBu | H | m-D7 | H | H | tBu | H | A-22 | H | H |
| 43827 | 1 | tBu | H | m-D7 | H | H | tBu | H | A-23 | H | H |
| 43828 | 1 | tBu | H | m-D7 | H | H | tBu | H | A-24 | H | H |
| 43829 | 1 | tBu | H | m-D7 | H | H | tBu | H | A-25 | H | H |
| 43830 | 1 | tBu | H | m-D7 | H | H | tBu | H | A-26 | H | H |
| 43831 | 1 | tBu | H | m-D7 | H | H | tBu | H | A-27 | H | H |
| 43832 | 1 | tBu | H | m-D7 | H | H | tBu | H | A-28 | H | H |
| 43833 | 1 | tBu | H | m-D7 | H | H | tBu | H | A-29 | H | H |
| 43834 | 1 | tBu | H | m-D7 | H | H | tBu | H | A-30 | H | H |
| 43835 | 1 | tBu | H | m-D7 | H | H | tBu | H | A-31 | H | H |
| 43836 | 1 | tBu | H | m-D7 | H | H | tBu | H | A-32 | H | H |
| 43837 | 1 | tBu | H | m-D7 | H | H | tBu | H | A-33 | H | H |
| 43838 | 1 | tBu | H | m-D7 | H | H | tBu | H | A-34 | H | H |
| 43839 | 1 | tBu | H | m-D7 | H | H | tBu | H | A-35 | H | H |
| 43840 | 1 | tBu | H | m-D7 | H | H | tBu | H | A-36 | H | H |
| 43841 | 1 | tBu | H | m-D7 | H | H | tBu | H | A-37 | H | H |
| 43842 | 1 | tBu | H | m-D7 | H | H | tBu | H | A-38 | H | H |
| 43843 | 1 | tBu | H | m-D7 | H | H | tBu | H | A-39 | H | H |
| 43844 | 1 | tBu | H | m-D7 | H | H | tBu | H | A-40 | H | H |
| 43845 | 1 | tBu | H | m-D7 | H | H | tBu | H | A-41 | H | H |
| 43846 | 1 | tBu | H | m-D7 | H | H | tBu | H | A-42 | H | H |
| 43847 | 1 | tBu | H | m-D7 | H | H | tBu | H | A-43 | H | H |
| 43848 | 1 | tBu | H | m-D7 | H | H | tBu | H | A-44 | H | H |
| 43849 | 1 | tBu | H | m-D7 | H | H | tBu | H | A-45 | H | H |
| 43850 | 1 | tBu | H | m-D7 | H | H | tBu | H | A-46 | H | H |
| 43851 | 1 | tBu | H | m-D7 | H | H | tBu | H | A-47 | H | H |
| 43852 | 1 | tBu | H | m-D8 | H | H | tBu | H | A-1 | H | H |
| 43853 | 1 | tBu | H | m-D8 | H | H | tBu | H | A-2 | H | H |
| 43854 | 1 | tBu | H | m-D8 | H | H | tBu | H | A-3 | H | H |
| 43855 | 1 | tBu | H | m-D8 | H | H | tBu | H | A-4 | H | H |
| 43856 | 1 | tBu | H | m-D8 | H | H | tBu | H | A-5 | H | H |
| 43857 | 1 | tBu | H | m-D8 | H | H | tBu | H | A-6 | H | H |
| 43858 | 1 | tBu | H | m-D8 | H | H | tBu | H | A-7 | H | H |
| 43859 | 1 | tBu | H | m-D8 | H | H | tBu | H | A-8 | H | H |
| 43860 | 1 | tBu | H | m-D8 | H | H | tBu | H | A-9 | H | H |
| 43861 | 1 | tBu | H | m-D8 | H | H | tBu | H | A-10 | H | H |
| 43862 | 1 | tBu | H | m-D8 | H | H | tBu | H | A-11 | H | H |
| 43863 | 1 | tBu | H | m-D8 | H | H | tBu | H | A-12 | H | H |
| 43864 | 1 | tBu | H | m-D8 | H | H | tBu | H | A-13 | H | H |
| 43865 | 1 | tBu | H | m-D8 | H | H | tBu | H | A-14 | H | H |
| 43866 | 1 | tBu | H | m-D8 | H | H | tBu | H | A-15 | H | H |
| 43867 | 1 | tBu | H | m-D8 | H | H | tBu | H | A-16 | H | H |
| 43868 | 1 | tBu | H | m-D8 | H | H | tBu | H | A-17 | H | H |
| 43869 | 1 | tBu | H | m-D8 | H | H | tBu | H | A-18 | H | H |
| 43870 | 1 | tBu | H | m-D8 | H | H | tBu | H | A-19 | H | H |
| 43871 | 1 | tBu | H | m-D8 | H | H | tBu | H | A-20 | H | H |
| 43872 | 1 | tBu | H | m-D8 | H | H | tBu | H | A-21 | H | H |
| 43873 | 1 | tBu | H | m-D8 | H | H | tBu | H | A-22 | H | H |
| 43874 | 1 | tBu | H | m-D8 | H | H | tBu | H | A-23 | H | H |
| 43875 | 1 | tBu | H | m-D8 | H | H | tBu | H | A-24 | H | H |
| 43876 | 1 | tBu | H | m-D8 | H | H | tBu | H | A-25 | H | H |
| 43877 | 1 | tBu | H | m-D8 | H | H | tBu | H | A-26 | H | H |
| 43878 | 1 | tBu | H | m-D8 | H | H | tBu | H | A-27 | H | H |
| 43879 | 1 | tBu | H | m-D8 | H | H | tBu | H | A-28 | H | H |
| 43880 | 1 | tBu | H | m-D8 | H | H | tBu | H | A-29 | H | H |
| 43881 | 1 | tBu | H | m-D8 | H | H | tBu | H | A-30 | H | H |
| 43882 | 1 | tBu | H | m-D8 | H | H | tBu | H | A-31 | H | H |
| 43883 | 1 | tBu | H | m-D8 | H | H | tBu | H | A-32 | H | H |
| 43884 | 1 | tBu | H | m-D8 | H | H | tBu | H | A-33 | H | H |
| 43885 | 1 | tBu | H | m-D8 | H | H | tBu | H | A-34 | H | H |
| 43886 | 1 | tBu | H | m-D8 | H | H | tBu | H | A-35 | H | H |
| 43887 | 1 | tBu | H | m-D8 | H | H | tBu | H | A-36 | H | H |
| 43888 | 1 | tBu | H | m-D8 | H | H | tBu | H | A-37 | H | H |
| 43889 | 1 | tBu | H | m-D8 | H | H | tBu | H | A-38 | H | H |
| 43890 | 1 | tBu | H | m-D8 | H | H | tBu | H | A-39 | H | H |
| 43891 | 1 | tBu | H | m-D8 | H | H | tBu | H | A-40 | H | H |
| 43892 | 1 | tBu | H | m-D8 | H | H | tBu | H | A-41 | H | H |
| 43893 | 1 | tBu | H | m-D8 | H | H | tBu | H | A-42 | H | H |
| 43894 | 1 | tBu | H | m-D8 | H | H | tBu | H | A-43 | H | H |
| 43895 | 1 | tBu | H | m-D8 | H | H | tBu | H | A-44 | H | H |
| 43896 | 1 | tBu | H | m-D8 | H | H | tBu | H | A-45 | H | H |
| 43897 | 1 | tBu | H | m-D8 | H | H | tBu | H | A-46 | H | H |
| 43898 | 1 | tBu | H | m-DB | H | H | tBu | H | A-47 | H | H |
| 43899 | 1 | tBu | H | m-D9 | H | H | tBu | H | A-1 | H | H |
| 43900 | 1 | tBu | H | m-D9 | H | H | tBu | H | A-2 | H | H |
| 43901 | 1 | tBu | H | m-D9 | H | H | tBu | H | A-3 | H | H |
| 43902 | 1 | tBu | H | m-D9 | H | H | tBu | H | A-4 | H | H |
| 43903 | 1 | tBu | H | m-D9 | H | H | tBu | H | A-5 | H | H |
| 43904 | 1 | tBu | H | m-D9 | H | H | tBu | H | A-6 | H | H |
| 43905 | 1 | tBu | H | m-D9 | H | H | tBu | H | A-7 | H | H |
| 43906 | 1 | tBu | H | m-D9 | H | H | tBu | H | A-8 | H | H |
| 43907 | 1 | tBu | H | m-D9 | H | H | tBu | H | A-9 | H | H |
| 43908 | 1 | tBu | H | m-D9 | H | H | tBu | H | A-10 | H | H |
| 43909 | 1 | tBu | H | m-D9 | H | H | tBu | H | A-11 | H | H |
| 43910 | 1 | tBu | H | m-D9 | H | H | tBu | H | A-12 | H | H |
| 43911 | 1 | tBu | H | m-D9 | H | H | tBu | H | A-13 | H | H |
| 43912 | 1 | tBu | H | m-D9 | H | H | tBu | H | A-14 | H | H |
| 43913 | 1 | tBu | H | m-D9 | H | H | tBu | H | A-15 | H | H |
| 43914 | 1 | tBu | H | m-D9 | H | H | tBu | H | A-16 | H | H |
| 43915 | 1 | tBu | H | m-D9 | H | H | tBu | H | A-17 | H | H |
| 43916 | 1 | tBu | H | m-D9 | H | H | tBu | H | A-18 | H | H |
| 43917 | 1 | tBu | H | m-D9 | H | H | tBu | H | A-19 | H | H |
| 43918 | 1 | tBu | H | m-D9 | H | H | tBu | H | A-20 | H | H |
| 43919 | 1 | tBu | H | m-D9 | H | H | tBu | H | A-21 | H | H |
| 43920 | 1 | tBu | H | m-D9 | H | H | tBu | H | A-22 | H | H |
| 43921 | 1 | tBu | H | m-D9 | H | H | tBu | H | A-23 | H | H |
| 43922 | 1 | tBu | H | m-D9 | H | H | tBu | H | A-24 | H | H |
| 43923 | 1 | tBu | H | m-D9 | H | H | tBu | H | A-25 | H | H |
| 43924 | 1 | tBu | H | m-D9 | H | H | tBu | H | A-26 | H | H |
| 43925 | 1 | tBu | H | m-D9 | H | H | tBu | H | A-27 | H | H |
| 43926 | 1 | tBu | H | m-D9 | H | H | tBu | H | A-28 | H | H |
| 43927 | 1 | tBu | H | m-D9 | H | H | tBu | H | A-29 | H | H |
| 43928 | 1 | tBu | H | m-D9 | H | H | tBu | H | A-30 | H | H |
| 43929 | 1 | tBu | H | m-D9 | H | H | tBu | H | A-31 | H | H |
| 43930 | 1 | tBu | H | m-D9 | H | H | tBu | H | A-32 | H | H |
| 43931 | 1 | tBu | H | m-D9 | H | H | tBu | H | A-33 | H | H |
| 43932 | 1 | tBu | H | m-D9 | H | H | tBu | H | A-34 | H | H |
| 43933 | 1 | tBu | H | m-D9 | H | H | tBu | H | A-35 | H | H |
| 43934 | 1 | tBu | H | m-D9 | H | H | tBu | H | A-36 | H | H |
| 43935 | 1 | tBu | H | m-D9 | H | H | tBu | H | A-37 | H | H |
| 43936 | 1 | tBu | H | m-D9 | H | H | tBu | H | A-38 | H | H |
| 43937 | 1 | tBu | H | m-D9 | H | H | tBu | H | A-39 | H | H |
| 43938 | 1 | tBu | H | m-D9 | H | H | tBu | H | A-40 | H | H |
| 43939 | 1 | tBu | H | m-D9 | H | H | tBu | H | A-41 | H | H |
| 43940 | 1 | tBu | H | m-D9 | H | H | tBu | H | A-42 | H | H |
| 43941 | 1 | tBu | H | m-D9 | H | H | tBu | H | A-43 | H | H |
| 43942 | 1 | tBu | H | m-D9 | H | H | tBu | H | A-44 | H | H |
| 43943 | 1 | tBu | H | m-D9 | H | H | tBu | H | A-45 | H | H |
| 43944 | 1 | tBu | H | m-D9 | H | H | tBu | H | A-46 | H | H |
| 43945 | 1 | tBu | H | m-D9 | H | H | tBu | H | A-47 | H | H |
| 43946 | 1 | tBu | H | m-D10 | H | H | tBu | H | A-1 | H | H |
| 43947 | 1 | tBu | H | m-D10 | H | H | tBu | H | A-2 | H | H |
| 43948 | 1 | tBu | H | m-D10 | H | H | tBu | H | A-3 | H | H |
| 43949 | 1 | tBu | H | m-D10 | H | H | tBu | H | A-4 | H | H |
| 43950 | 1 | tBu | H | m-D10 | H | H | tBu | H | A-5 | H | H |
| 43951 | 1 | tBu | H | m-D10 | H | H | tBu | H | A-6 | H | H |
| 43952 | 1 | tBu | H | m-D10 | H | H | tBu | H | A-7 | H | H |
| 43953 | 1 | tBu | H | m-D10 | H | H | tBu | H | A-8 | H | H |
| 43954 | 1 | tBu | H | m-D10 | H | H | tBu | H | A-9 | H | H |
| 43955 | 1 | tBu | H | m-D10 | H | H | tBu | H | A-10 | H | H |
| 43956 | 1 | tBu | H | m-D10 | H | H | tBu | H | A-11 | H | H |
| 43957 | 1 | tBu | H | m-D10 | H | H | tBu | H | A-12 | H | H |
| 43958 | 1 | tBu | H | m-D10 | H | H | tBu | H | A-13 | H | H |
| 43959 | 1 | tBu | H | m-D10 | H | H | tBu | H | A-14 | H | H |
| 43960 | 1 | tBu | H | m-D10 | H | H | tBu | H | A-15 | H | H |
| 43961 | 1 | tBu | H | m-D10 | H | H | tBu | H | A-16 | H | H |
| 43962 | 1 | tBu | H | m-D10 | H | H | tBu | H | A-17 | H | H |
| 43963 | 1 | tBu | H | m-D10 | H | H | tBu | H | A-18 | H | H |
| 43964 | 1 | tBu | H | m-D10 | H | H | tBu | H | A-19 | H | H |
| 43965 | 1 | tBu | H | m-D10 | H | H | tBu | H | A-20 | H | H |
| 43966 | 1 | tBu | H | m-D10 | H | H | tBu | H | A-21 | H | H |
| 43967 | 1 | tBu | H | m-D10 | H | H | tBu | H | A-22 | H | H |
| 43968 | 1 | tBu | H | m-D10 | H | H | tBu | H | A-23 | H | H |
| 43969 | 1 | tBu | H | m-D10 | H | H | tBu | H | A-24 | H | H |
| 43970 | 1 | tBu | H | m-D10 | H | H | tBu | H | A-25 | H | H |
| 43971 | 1 | tBu | H | m-D10 | H | H | tBu | H | A-26 | H | H |
| 43972 | 1 | tBu | H | m-D10 | H | H | tBu | H | A-27 | H | H |
| 43973 | 1 | tBu | H | m-D10 | H | H | tBu | H | A-28 | H | H |
| 43974 | 1 | tBu | H | m-D10 | H | H | tBu | H | A-29 | H | H |
| 43975 | 1 | tBu | H | m-D10 | H | H | tBu | H | A-30 | H | H |
| 43976 | 1 | tBu | H | m-D10 | H | H | tBu | H | A-31 | H | H |
| 43977 | 1 | tBu | H | m-D10 | H | H | tBu | H | A-32 | H | H |
| 43978 | 1 | tBu | H | m-D10 | H | H | tBu | H | A-33 | H | H |
| 43979 | 1 | tBu | H | m-D10 | H | H | tBu | H | A-34 | H | H |
| 43980 | 1 | tBu | H | m-D10 | H | H | tBu | H | A-35 | H | H |
| 43981 | 1 | tBu | H | m-D10 | H | H | tBu | H | A-36 | H | H |
| 43982 | 1 | tBu | H | m-D10 | H | H | tBu | H | A-37 | H | H |
| 43983 | 1 | tBu | H | m-D10 | H | H | tBu | H | A-38 | H | H |
| 43984 | 1 | tBu | H | m-D10 | H | H | tBu | H | A-39 | H | H |
| 43985 | 1 | tBu | H | m-D10 | H | H | tBu | H | A-40 | H | H |
| 43986 | 1 | tBu | H | m-D10 | H | H | tBu | H | A-41 | H | H |
| 43987 | 1 | tBu | H | m-D10 | H | H | tBu | H | A-42 | H | H |
| 43988 | 1 | tBu | H | m-D10 | H | H | tBu | H | A-43 | H | H |
| 43989 | 1 | tBu | H | m-D10 | H | H | tBu | H | A-44 | H | H |
| 43990 | 1 | tBu | H | m-D10 | H | H | tBu | H | A-45 | H | H |
| 43991 | 1 | tBu | H | m-D10 | H | H | tBu | H | A-46 | H | H |
| 43992 | 1 | tBu | H | m-D10 | H | H | tBu | H | A-47 | H | H |
| 43993 | 1 | tBu | H | m-D11 | H | H | tBu | H | A-1 | H | H |
| 43994 | 1 | tBu | H | m-D11 | H | H | tBu | H | A-2 | H | H |
| 43995 | 1 | tBu | H | m-D11 | H | H | tBu | H | A-3 | H | H |
| 43996 | 1 | tBu | H | m-D11 | H | H | tBu | H | A-4 | H | H |
| 43997 | 1 | tBu | H | m-D11 | H | H | tBu | H | A-5 | H | H |
| 43998 | 1 | tBu | H | m-D11 | H | H | tBu | H | A-6 | H | H |
| 43999 | 1 | tBu | H | m-D11 | H | H | tBu | H | A-7 | H | H |
| 44000 | 1 | tBu | H | m-D11 | H | H | tBu | H | A-8 | H | H |
| 44001 | 1 | tBu | H | m-D11 | H | H | tBu | H | A-9 | H | H |
| 44002 | 1 | tBu | H | m-D11 | H | H | tBu | H | A-10 | H | H |
| 44003 | 1 | tBu | H | m-D11 | H | H | tBu | H | A-11 | H | H |
| 44004 | 1 | tBu | H | m-D11 | H | H | tBu | H | A-12 | H | H |
| 44005 | 1 | tBu | H | m-D11 | H | H | tBu | H | A-13 | H | H |
| 44006 | 1 | tBu | H | m-D11 | H | H | tBu | H | A-14 | H | H |
| 44007 | 1 | tBu | H | m-D11 | H | H | tBu | H | A-15 | H | H |
| 44008 | 1 | tBu | H | m-D11 | H | H | tBu | H | A-16 | H | H |
| 44009 | 1 | tBu | H | m-D11 | H | H | tBu | H | A-17 | H | H |
| 44010 | 1 | tBu | H | m-D11 | H | H | tBu | H | A-18 | H | H |
| 44011 | 1 | tBu | H | m-D11 | H | H | tBu | H | A-19 | H | H |
| 44012 | 1 | tBu | H | m-D11 | H | H | tBu | H | A-20 | H | H |
| 44013 | 1 | tBu | H | m-D11 | H | H | tBu | H | A-21 | H | H |
| 44014 | 1 | tBu | H | m-D11 | H | H | tBu | H | A-22 | H | H |
| 44015 | 1 | tBu | H | m-D11 | H | H | tBu | H | A-23 | H | H |
| 44016 | 1 | tBu | H | m-D11 | H | H | tBu | H | A-24 | H | H |
| 44017 | 1 | tBu | H | m-D11 | H | H | tBu | H | A-25 | H | H |
| 44018 | 1 | tBu | H | m-D11 | H | H | tBu | H | A-26 | H | H |
| 44019 | 1 | tBu | H | m-D11 | H | H | tBu | H | A-27 | H | H |
| 44020 | 1 | tBu | H | m-D11 | H | H | tBu | H | A-28 | H | H |
| 44021 | 1 | tBu | H | m-D11 | H | H | tBu | H | A-29 | H | H |
| 44022 | 1 | tBu | H | m-D11 | H | H | tBu | H | A-30 | H | H |
| 44023 | 1 | tBu | H | m-D11 | H | H | tBu | H | A-31 | H | H |
| 44024 | 1 | tBu | H | m-D11 | H | H | tBu | H | A-32 | H | H |
| 44025 | 1 | tBu | H | m-D11 | H | H | tBu | H | A-33 | H | H |
| 44026 | 1 | tBu | H | m-D11 | H | H | tBu | H | A-34 | H | H |
| 44027 | 1 | tBu | H | m-D11 | H | H | tBu | H | A-35 | H | H |
| 44028 | 1 | tBu | H | m-D11 | H | H | tBu | H | A-36 | H | H |
| 44029 | 1 | tBu | H | m-D11 | H | H | tBu | H | A-37 | H | H |
| 44030 | 1 | tBu | H | m-D11 | H | H | tBu | H | A-38 | H | H |
| 44031 | 1 | tBu | H | m-D11 | H | H | tBu | H | A-39 | H | H |
| 44032 | 1 | tBu | H | m-D11 | H | H | tBu | H | A-40 | H | H |
| 44033 | 1 | tBu | H | m-D11 | H | H | tBu | H | A-41 | H | H |
| 44034 | 1 | tBu | H | m-D11 | H | H | tBu | H | A-42 | H | H |
| 44035 | 1 | tBu | H | m-D11 | H | H | tBu | H | A-43 | H | H |
| 44036 | 1 | tBu | H | m-D11 | H | H | tBu | H | A-44 | H | H |
| 44037 | 1 | tBu | H | m-D11 | H | H | tBu | H | A-45 | H | H |
| 44038 | 1 | tBu | H | m-D11 | H | H | tBu | H | A-46 | H | H |
| 44039 | 1 | tBu | H | m-D11 | H | H | tBu | H | A-47 | H | H |
| 44040 | 1 | tBu | H | m-D12 | H | H | tBu | H | A-1 | H | H |
| 44041 | 1 | tBu | H | m-D12 | H | H | tBu | H | A-2 | H | H |
| 44042 | 1 | tBu | H | m-D12 | H | H | tBu | H | A-3 | H | H |
| 44043 | 1 | tBu | H | m-D12 | H | H | tBu | H | A-4 | H | H |
| 44044 | 1 | tBu | H | m-D12 | H | H | tBu | H | A-5 | H | H |
| 44045 | 1 | tBu | H | m-D12 | H | H | tBu | H | A-6 | H | H |
| 44046 | 1 | tBu | H | m-D12 | H | H | tBu | H | A-7 | H | H |
| 44047 | 1 | tBu | H | m-D12 | H | H | tBu | H | A-8 | H | H |
| 44048 | 1 | tBu | H | m-D12 | H | H | tBu | H | A-9 | H | H |
| 44049 | 1 | tBu | H | m-D12 | H | H | tBu | H | A-10 | H | H |
| 44050 | 1 | tBu | H | m-D12 | H | H | tBu | H | A-11 | H | H |
| 44051 | 1 | tBu | H | m-D12 | H | H | tBu | H | A-12 | H | H |
| 44052 | 1 | tBu | H | m-D12 | H | H | tBu | H | A-13 | H | H |
| 44053 | 1 | tBu | H | m-D12 | H | H | tBu | H | A-14 | H | H |
| 44054 | 1 | tBu | H | m-D12 | H | H | tBu | H | A-15 | H | H |
| 44055 | 1 | tBu | H | m-D12 | H | H | tBu | H | A-16 | H | H |
| 44056 | 1 | tBu | H | m-D12 | H | H | tBu | H | A-17 | H | H |
| 44057 | 1 | tBu | H | m-D12 | H | H | tBu | H | A-18 | H | H |
| 44058 | 1 | tBu | H | m-D12 | H | H | tBu | H | A-19 | H | H |
| 44059 | 1 | tBu | H | m-D12 | H | H | tBu | H | A-20 | H | H |
| 44060 | 1 | tBu | H | m-D12 | H | H | tBu | H | A-21 | H | H |
| 44061 | 1 | tBu | H | m-D12 | H | H | tBu | H | A-22 | H | H |
| 44062 | 1 | tBu | H | m-D12 | H | H | tBu | H | A-23 | H | H |
| 44063 | 1 | tBu | H | m-D12 | H | H | tBu | H | A-24 | H | H |
| 44064 | 1 | tBu | H | m-D12 | H | H | tBu | H | A-25 | H | H |
| 44065 | 1 | tBu | H | m-D12 | H | H | tBu | H | A-26 | H | H |
| 44066 | 1 | tBu | H | m-D12 | H | H | tBu | H | A-27 | H | H |
| 44067 | 1 | tBu | H | m-D12 | H | H | tBu | H | A-28 | H | H |
| 44068 | 1 | tBu | H | m-D12 | H | H | tBu | H | A-29 | H | H |
| 44069 | 1 | tBu | H | m-D12 | H | H | tBu | H | A-30 | H | H |
| 44070 | 1 | tBu | H | m-D12 | H | H | tBu | H | A-31 | H | H |
| 44071 | 1 | tBu | H | m-D12 | H | H | tBu | H | A-32 | H | H |
| 44072 | 1 | tBu | H | m-D12 | H | H | tBu | H | A-33 | H | H |
| 44073 | 1 | tBu | H | m-D12 | H | H | tBu | H | A-34 | H | H |
| 44074 | 1 | tBu | H | m-D12 | H | H | tBu | H | A-35 | H | H |
| 44075 | 1 | tBu | H | m-D12 | H | H | tBu | H | A-36 | H | H |
| 44076 | 1 | tBu | H | m-D12 | H | H | tBu | H | A-37 | H | H |
| 44077 | 1 | tBu | H | m-D12 | H | H | tBu | H | A-38 | H | H |
| 44078 | 1 | tBu | H | m-D12 | H | H | tBu | H | A-39 | H | H |
| 44079 | 1 | tBu | H | m-D12 | H | H | tBu | H | A-40 | H | H |
| 44080 | 1 | tBu | H | m-D12 | H | H | tBu | H | A-41 | H | H |
| 44081 | 1 | tBu | H | m-D12 | H | H | tBu | H | A-42 | H | H |
| 44082 | 1 | tBu | H | m-D12 | H | H | tBu | H | A-43 | H | H |
| 44083 | 1 | tBu | H | m-D12 | H | H | tBu | H | A-44 | H | H |
| 44084 | 1 | tBu | H | m-D12 | H | H | tBu | H | A-45 | H | H |
| 44085 | 1 | tBu | H | m-D12 | H | H | tBu | H | A-46 | H | H |
| 44086 | 1 | tBu | H | m-D12 | H | H | tBu | H | A-47 | H | H |
| 44087 | 1 | tBu | H | m-D13 | H | H | tBu | H | A-1 | H | H |
| 44088 | 1 | tBu | H | m-D13 | H | H | tBu | H | A-2 | H | H |
| 44089 | 1 | tBu | H | m-D13 | H | H | tBu | H | A-3 | H | H |
| 44090 | 1 | tBu | H | m-D13 | H | H | tBu | H | A-4 | H | H |
| 44091 | 1 | tBu | H | m-D13 | H | H | tBu | H | A-5 | H | H |
| 44092 | 1 | tBu | H | m-D13 | H | H | tBu | H | A-6 | H | H |
| 44093 | 1 | tBu | H | m-D13 | H | H | tBu | H | A-7 | H | H |
| 44094 | 1 | tBu | H | m-D13 | H | H | tBu | H | A-8 | H | H |
| 44095 | 1 | tBu | H | m-D13 | H | H | tBu | H | A-9 | H | H |
| 44096 | 1 | tBu | H | m-D13 | H | H | tBu | H | A-10 | H | H |
| 44097 | 1 | tBu | H | m-D13 | H | H | tBu | H | A-11 | H | H |
| 44098 | 1 | tBu | H | m-D13 | H | H | tBu | H | A-12 | H | H |
| 44099 | 1 | tBu | H | m-D13 | H | H | tBu | H | A-13 | H | H |
| 44100 | 1 | tBu | H | m-D13 | H | H | tBu | H | A-14 | H | H |
| 44101 | 1 | tBu | H | m-D13 | H | H | tBu | H | A-15 | H | H |
| 44102 | 1 | tBu | H | m-D13 | H | H | tBu | H | A-16 | H | H |
| 44103 | 1 | tBu | H | m-D13 | H | H | tBu | H | A-17 | H | H |
| 44104 | 1 | tBu | H | m-D13 | H | H | tBu | H | A-18 | H | H |
| 44105 | 1 | tBu | H | m-D13 | H | H | tBu | H | A-19 | H | H |
| 44106 | 1 | tBu | H | m-D13 | H | H | tBu | H | A-20 | H | H |
| 44107 | 1 | tBu | H | m-D13 | H | H | tBu | H | A-21 | H | H |
| 44108 | 1 | tBu | H | m-D13 | H | H | tBu | H | A-22 | H | H |
| 44109 | 1 | tBu | H | m-D13 | H | H | tBu | H | A-23 | H | H |
| 44110 | 1 | tBu | H | m-D13 | H | H | tBu | H | A-24 | H | H |
| 44111 | 1 | tBu | H | m-D13 | H | H | tBu | H | A-25 | H | H |
| 44112 | 1 | tBu | H | m-D13 | H | H | tBu | H | A-26 | H | H |
| 44113 | 1 | tBu | H | m-D13 | H | H | tBu | H | A-27 | H | H |
| 44114 | 1 | tBu | H | m-D13 | H | H | tBu | H | A-28 | H | H |
| 44115 | 1 | tBu | H | m-D13 | H | H | tBu | H | A-29 | H | H |
| 44116 | 1 | tBu | H | m-D13 | H | H | tBu | H | A-30 | H | H |
| 44117 | 1 | tBu | H | m-D13 | H | H | tBu | H | A-31 | H | H |
| 44118 | 1 | tBu | H | m-D13 | H | H | tBu | H | A-32 | H | H |
| 44119 | 1 | tBu | H | m-D13 | H | H | tBu | H | A-33 | H | H |
| 44120 | 1 | tBu | H | m-D13 | H | H | tBu | H | A-34 | H | H |
| 44121 | 1 | tBu | H | m-D13 | H | H | tBu | H | A-35 | H | H |
| 44122 | 1 | tBu | H | m-D13 | H | H | tBu | H | A-36 | H | H |
| 44123 | 1 | tBu | H | m-D13 | H | H | tBu | H | A-37 | H | H |
| 44124 | 1 | tBu | H | m-D13 | H | H | tBu | H | A-38 | H | H |
| 44125 | 1 | tBu | H | m-D13 | H | H | tBu | H | A-39 | H | H |
| 44126 | 1 | tBu | H | m-D13 | H | H | tBu | H | A-40 | H | H |
| 44127 | 1 | tBu | H | m-D13 | H | H | tBu | H | A-41 | H | H |
| 44128 | 1 | tBu | H | m-D13 | H | H | tBu | H | A-42 | H | H |
| 44129 | 1 | tBu | H | m-D13 | H | H | tBu | H | A-43 | H | H |
| 44130 | 1 | tBu | H | m-D13 | H | H | tBu | H | A-44 | H | H |
| 44131 | 1 | tBu | H | m-D13 | H | H | tBu | H | A-45 | H | H |
| 44132 | 1 | tBu | H | m-D13 | H | H | tBu | H | A-46 | H | H |
| 44133 | 1 | tBu | H | m-D13 | H | H | tBu | H | A-47 | H | H |
| 44134 | 1 | tBu | H | m-D14 | H | H | tBu | H | A-1 | H | H |
| 44135 | 1 | tBu | H | m-D14 | H | H | tBu | H | A-2 | H | H |
| 44136 | 1 | tBu | H | m-D14 | H | H | tBu | H | A-3 | H | H |
| 44137 | 1 | tBu | H | m-D14 | H | H | tBu | H | A-4 | H | H |
| 44138 | 1 | tBu | H | m-D14 | H | H | tBu | H | A-5 | H | H |
| 44139 | 1 | tBu | H | m-D14 | H | H | tBu | H | A-6 | H | H |
| 44140 | 1 | tBu | H | m-D14 | H | H | tBu | H | A-7 | H | H |
| 44141 | 1 | tBu | H | m-D14 | H | H | tBu | H | A-8 | H | H |
| 44142 | 1 | tBu | H | m-D14 | H | H | tBu | H | A-9 | H | H |
| 44143 | 1 | tBu | H | m-D14 | H | H | tBu | H | A-10 | H | H |
| 44144 | 1 | tBu | H | m-D14 | H | H | tBu | H | A-11 | H | H |
| 44145 | 1 | tBu | H | m-D14 | H | H | tBu | H | A-12 | H | H |
| 44146 | 1 | tBu | H | m-D14 | H | H | tBu | H | A-13 | H | H |
| 44147 | 1 | tBu | H | m-D14 | H | H | tBu | H | A-14 | H | H |
| 44148 | 1 | tBu | H | m-D14 | H | H | tBu | H | A-15 | H | H |
| 44149 | 1 | tBu | H | m-D14 | H | H | tBu | H | A-16 | H | H |
| 44150 | 1 | tBu | H | m-D14 | H | H | tBu | H | A-17 | H | H |
| 44151 | 1 | tBu | H | m-D14 | H | H | tBu | H | A-18 | H | H |
| 44152 | 1 | tBu | H | m-D14 | H | H | tBu | H | A-19 | H | H |
| 44153 | 1 | tBu | H | m-D14 | H | H | tBu | H | A-20 | H | H |
| 44154 | 1 | tBu | H | m-D14 | H | H | tBu | H | A-21 | H | H |
| 44155 | 1 | tBu | H | m-D14 | H | H | tBu | H | A-22 | H | H |
| 44156 | 1 | tBu | H | m-D14 | H | H | tBu | H | A-23 | H | H |
| 44157 | 1 | tBu | H | m-D14 | H | H | tBu | H | A-24 | H | H |
| 44158 | 1 | tBu | H | m-D14 | H | H | tBu | H | A-25 | H | H |
| 44159 | 1 | tBu | H | m-D14 | H | H | tBu | H | A-26 | H | H |
| 44160 | 1 | tBu | H | m-D14 | H | H | tBu | H | A-27 | H | H |
| 44161 | 1 | tBu | H | m-D14 | H | H | tBu | H | A-28 | H | H |
| 44162 | 1 | tBu | H | m-D14 | H | H | tBu | H | A-29 | H | H |
| 44163 | 1 | tBu | H | m-D14 | H | H | tBu | H | A-30 | H | H |
| 44164 | 1 | tBu | H | m-D14 | H | H | tBu | H | A-31 | H | H |
| 44165 | 1 | tBu | H | m-D14 | H | H | tBu | H | A-32 | H | H |
| 44166 | 1 | tBu | H | m-D14 | H | H | tBu | H | A-33 | H | H |
| 44167 | 1 | tBu | H | m-D14 | H | H | tBu | H | A-34 | H | H |
| 44168 | 1 | tBu | H | m-D14 | H | H | tBu | H | A-35 | H | H |
| 44169 | 1 | tBu | H | m-D14 | H | H | tBu | H | A-36 | H | H |
| 44170 | 1 | tBu | H | m-D14 | H | H | tBu | H | A-37 | H | H |
| 44171 | 1 | tBu | H | m-D14 | H | H | tBu | H | A-38 | H | H |
| 44172 | 1 | tBu | H | m-D14 | H | H | tBu | H | A-39 | H | H |
| 44173 | 1 | tBu | H | m-D14 | H | H | tBu | H | A-40 | H | H |
| 44174 | 1 | tBu | H | m-D14 | H | H | tBu | H | A-41 | H | H |
| 44175 | 1 | tBu | H | m-D14 | H | H | tBu | H | A-42 | H | H |
| 44176 | 1 | tBu | H | m-D14 | H | H | tBu | H | A-43 | H | H |
| 44177 | 1 | tBu | H | m-D14 | H | H | tBu | H | A-44 | H | H |
| 44178 | 1 | tBu | H | m-D14 | H | H | tBu | H | A-45 | H | H |
| 44179 | 1 | tBu | H | m-D14 | H | H | tBu | H | A-46 | H | H |
| 44180 | 1 | tBu | H | m-D14 | H | H | tBu | H | A-47 | H | H |
| 44181 | 1 | H | m-D1 | H | m-D1 | H | tBu | A-1 | H | A-1 | H |
| 44182 | 1 | H | m-D1 | H | m-D1 | H | tBu | A-2 | H | A-2 | H |
| 44183 | 1 | H | m-D1 | H | m-D1 | H | tBu | A-3 | H | A-3 | H |
| 44184 | 1 | H | m-D1 | H | m-D1 | H | tBu | A-4 | H | A-4 | H |
| 44185 | 1 | H | m-D1 | H | m-D1 | H | tBu | A-5 | H | A-5 | H |
| 44186 | 1 | H | m-D1 | H | m-D1 | H | tBu | A-6 | H | A-6 | H |
| 44187 | 1 | H | m-D1 | H | m-D1 | H | tBu | A-7 | H | A-7 | H |
| 44188 | 1 | H | m-D1 | H | m-D1 | H | tBu | A-8 | H | A-8 | H |
| 44189 | 1 | H | m-D1 | H | m-D1 | H | tBu | A-9 | H | A-9 | H |
| 44190 | 1 | H | m-D1 | H | m-D1 | H | tBu | A-10 | H | A-10 | H |
| 44191 | 1 | H | m-D1 | H | m-D1 | H | tBu | A-11 | H | A-11 | H |
| 44192 | 1 | H | m-D1 | H | m-D1 | H | tBu | A-12 | H | A-12 | H |
| 44193 | 1 | H | m-D1 | H | m-D1 | H | tBu | A-13 | H | A-13 | H |
| 44194 | 1 | H | m-D1 | H | m-D1 | H | tBu | A-14 | H | A-14 | H |
| 44195 | 1 | H | m-D1 | H | m-D1 | H | tBu | A-15 | H | A-15 | H |
| 44196 | 1 | H | m-D1 | H | m-D1 | H | tBu | A-16 | H | A-16 | H |
| 44197 | 1 | H | m-D1 | H | m-D1 | H | tBu | A-17 | H | A-17 | H |
| 44198 | 1 | H | m-D1 | H | m-D1 | H | tBu | A-18 | H | A-18 | H |
| 44199 | 1 | H | m-D1 | H | m-D1 | H | tBu | A-19 | H | A-19 | H |
| 44200 | 1 | H | m-D1 | H | m-D1 | H | tBu | A-20 | H | A-20 | H |
| 44201 | 1 | H | m-D1 | H | m-D1 | H | tBu | A-21 | H | A-21 | H |
| 44202 | 1 | H | m-D1 | H | m-D1 | H | tBu | A-22 | H | A-22 | H |
| 44203 | 1 | H | m-D1 | H | m-D1 | H | tBu | A-23 | H | A-23 | H |
| 44204 | 1 | H | m-D1 | H | m-D1 | H | tBu | A-24 | H | A-24 | H |
| 44205 | 1 | H | m-D1 | H | m-D1 | H | tBu | A-25 | H | A-25 | H |
| 44206 | 1 | H | m-D1 | H | m-D1 | H | tBu | A-26 | H | A-26 | H |
| 44207 | 1 | H | m-D1 | H | m-D1 | H | tBu | A-27 | H | A-27 | H |
| 44208 | 1 | H | m-D1 | H | m-D1 | H | tBu | A-28 | H | A-28 | H |
| 44209 | 1 | H | m-D1 | H | m-D1 | H | tBu | A-29 | H | A-29 | H |
| 44210 | 1 | H | m-D1 | H | m-D1 | H | tBu | A-30 | H | A-30 | H |
| 44211 | 1 | H | m-D1 | H | m-D1 | H | tBu | A-31 | H | A-31 | H |
| 44212 | 1 | H | m-D1 | H | m-D1 | H | tBu | A-32 | H | A-32 | H |
| 44213 | 1 | H | m-D1 | H | m-D1 | H | tBu | A-33 | H | A-33 | H |
| 44214 | 1 | H | m-D1 | H | m-D1 | H | tBu | A-34 | H | A-34 | H |
| 44215 | 1 | H | m-D1 | H | m-D1 | H | tBu | A-35 | H | A-35 | H |
| 44216 | 1 | H | m-D1 | H | m-D1 | H | tBu | A-36 | H | A-36 | H |
| 44217 | 1 | H | m-D1 | H | m-D1 | H | tBu | A-37 | H | A-37 | H |
| 44218 | 1 | H | m-D1 | H | m-D1 | H | tBu | A-38 | H | A-38 | H |
| 44219 | 1 | H | m-D1 | H | m-D1 | H | tBu | A-39 | H | A-39 | H |
| 44220 | 1 | H | m-D1 | H | m-D1 | H | tBu | A-40 | H | A-40 | H |
| 44221 | 1 | H | m-D1 | H | m-D1 | H | tBu | A-41 | H | A-41 | H |
| 44222 | 1 | H | m-D1 | H | m-D1 | H | tBu | A-42 | H | A-42 | H |
| 44223 | 1 | H | m-D1 | H | m-D1 | H | tBu | A-43 | H | A-43 | H |
| 44224 | 1 | H | m-D1 | H | m-D1 | H | tBu | A-44 | H | A-44 | H |
| 44225 | 1 | H | m-D1 | H | m-D1 | H | tBu | A-45 | H | A-45 | H |
| 44226 | 1 | H | m-D1 | H | m-D1 | H | tBu | A-46 | H | A-46 | H |
| 44227 | 1 | H | m-D1 | H | m-D1 | H | tBu | A-47 | H | A-47 | H |
| 44228 | 1 | H | m-D2 | H | m-D2 | H | tBu | A-1 | H | A-1 | H |
| 44229 | 1 | H | m-D2 | H | m-D2 | H | tBu | A-2 | H | A-2 | H |
| 44230 | 1 | H | m-D2 | H | m-D2 | H | tBu | A-3 | H | A-3 | H |
| 44231 | 1 | H | m-D2 | H | m-D2 | H | tBu | A-4 | H | A-4 | H |
| 44232 | 1 | H | m-D2 | H | m-D2 | H | tBu | A-5 | H | A-5 | H |
| 44233 | 1 | H | m-D2 | H | m-D2 | H | tBu | A-6 | H | A-6 | H |
| 44234 | 1 | H | m-D2 | H | m-D2 | H | tBu | A-7 | H | A-7 | H |
| 44235 | 1 | H | m-D2 | H | m-D2 | H | tBu | A-8 | H | A-8 | H |
| 44236 | 1 | H | m-D2 | H | m-D2 | H | tBu | A-9 | H | A-9 | H |
| 44237 | 1 | H | m-D2 | H | m-D2 | H | tBu | A-10 | H | A-10 | H |
| 44238 | 1 | H | m-D2 | H | m-D2 | H | tBu | A-11 | H | A-11 | H |
| 44239 | 1 | H | m-D2 | H | m-D2 | H | tBu | A-12 | H | A-12 | H |
| 44240 | 1 | H | m-D2 | H | m-D2 | H | tBu | A-13 | H | A-13 | H |
| 44241 | 1 | H | m-D2 | H | m-D2 | H | tBu | A-14 | H | A-14 | H |
| 44242 | 1 | H | m-D2 | H | m-D2 | H | tBu | A-15 | H | A-15 | H |
| 44243 | 1 | H | m-D2 | H | m-D2 | H | tBu | A-16 | H | A-16 | H |
| 44244 | 1 | H | m-D2 | H | m-D2 | H | tBu | A-17 | H | A-17 | H |
| 44245 | 1 | H | m-D2 | H | m-D2 | H | tBu | A-18 | H | A-18 | H |
| 44246 | 1 | H | m-D2 | H | m-D2 | H | tBu | A-19 | H | A-19 | H |
| 44247 | 1 | H | m-D2 | H | m-D2 | H | tBu | A-20 | H | A-20 | H |
| 44248 | 1 | H | m-D2 | H | m-D2 | H | tBu | A-21 | H | A-21 | H |
| 44249 | 1 | H | m-D2 | H | m-D2 | H | tBu | A-22 | H | A-22 | H |
| 44250 | 1 | H | m-D2 | H | m-D2 | H | tBu | A-23 | H | A-23 | H |
| 44251 | 1 | H | m-D2 | H | m-D2 | H | tBu | A-24 | H | A-24 | H |
| 44252 | 1 | H | m-D2 | H | m-D2 | H | tBu | A-25 | H | A-25 | H |
| 44253 | 1 | H | m-D2 | H | m-D2 | H | tBu | A-26 | H | A-26 | H |
| 44254 | 1 | H | m-D2 | H | m-D2 | H | tBu | A-27 | H | A-27 | H |
| 44255 | 1 | H | m-D2 | H | m-D2 | H | tBu | A-28 | H | A-28 | H |
| 44256 | 1 | H | m-D2 | H | m-D2 | H | tBu | A-29 | H | A-29 | H |
| 44257 | 1 | H | m-D2 | H | m-D2 | H | tBu | A-30 | H | A-30 | H |
| 44258 | 1 | H | m-D2 | H | m-D2 | H | tBu | A-31 | H | A-31 | H |
| 44259 | 1 | H | m-D2 | H | m-D2 | H | tBu | A-32 | H | A-32 | H |
| 44260 | 1 | H | m-D2 | H | m-D2 | H | tBu | A-33 | H | A-33 | H |
| 44261 | 1 | H | m-D2 | H | m-D2 | H | tBu | A-34 | H | A-34 | H |
| 44262 | 1 | H | m-D2 | H | m-D2 | H | tBu | A-35 | H | A-35 | H |
| 44263 | 1 | H | m-D2 | H | m-D2 | H | tBu | A-36 | H | A-36 | H |
| 44264 | 1 | H | m-D2 | H | m-D2 | H | tBu | A-37 | H | A-37 | H |
| 44265 | 1 | H | m-D2 | H | m-D2 | H | tBu | A-38 | H | A-38 | H |
| 44266 | 1 | H | m-D2 | H | m-D2 | H | tBu | A-39 | H | A-39 | H |
| 44267 | 1 | H | m-D2 | H | m-D2 | H | tBu | A-40 | H | A-40 | H |
| 44268 | 1 | H | m-D2 | H | m-D2 | H | tBu | A-41 | H | A-41 | H |
| 44269 | 1 | H | m-D2 | H | m-D2 | H | tBu | A-42 | H | A-42 | H |
| 44270 | 1 | H | m-D2 | H | m-D2 | H | tBu | A-43 | H | A-43 | H |
| 44271 | 1 | H | m-D2 | H | m-D2 | H | tBu | A-44 | H | A-44 | H |
| 44272 | 1 | H | m-D2 | H | m-D2 | H | tBu | A-45 | H | A-45 | H |
| 44273 | 1 | H | m-D2 | H | m-D2 | H | tBu | A-46 | H | A-46 | H |
| 44274 | 1 | H | m-D2 | H | m-D2 | H | tBu | A-47 | H | A-47 | H |
| 44275 | 1 | H | m-D3 | H | m-D3 | H | tBu | A-1 | H | A-1 | H |
| 44276 | 1 | H | m-D3 | H | m-D3 | H | tBu | A-2 | H | A-2 | H |
| 44277 | 1 | H | m-D3 | H | m-D3 | H | tBu | A-3 | H | A-3 | H |
| 44278 | 1 | H | m-D3 | H | m-D3 | H | tBu | A-4 | H | A-4 | H |
| 44279 | 1 | H | m-D3 | H | m-D3 | H | tBu | A-5 | H | A-5 | H |
| 44280 | 1 | H | m-D3 | H | m-D3 | H | tBu | A-6 | H | A-6 | H |
| 44281 | 1 | H | m-D3 | H | m-D3 | H | tBu | A-7 | H | A-7 | H |
| 44282 | 1 | H | m-D3 | H | m-D3 | H | tBu | A-8 | H | A-8 | H |
| 44283 | 1 | H | m-D3 | H | m-D3 | H | tBu | A-9 | H | A-9 | H |
| 44284 | 1 | H | m-D3 | H | m-D3 | H | tBu | A-10 | H | A-10 | H |
| 44285 | 1 | H | m-D3 | H | m-D3 | H | tBu | A-11 | H | A-11 | H |
| 44286 | 1 | H | m-D3 | H | m-D3 | H | tBu | A-12 | H | A-12 | H |
| 44287 | 1 | H | m-D3 | H | m-D3 | H | tBu | A-13 | H | A-13 | H |
| 44288 | 1 | H | m-D3 | H | m-D3 | H | tBu | A-14 | H | A-14 | H |
| 44289 | 1 | H | m-D3 | H | m-D3 | H | tBu | A-15 | H | A-15 | H |
| 44290 | 1 | H | m-D3 | H | m-D3 | H | tBu | A-16 | H | A-16 | H |
| 44291 | 1 | H | m-D3 | H | m-D3 | H | tBu | A-17 | H | A-17 | H |
| 44292 | 1 | H | m-D3 | H | m-D3 | H | tBu | A-18 | H | A-18 | H |
| 44293 | 1 | H | m-D3 | H | m-D3 | H | tBu | A-19 | H | A-19 | H |
| 44294 | 1 | H | m-D3 | H | m-D3 | H | tBu | A-20 | H | A-20 | H |
| 44295 | 1 | H | m-D3 | H | m-D3 | H | tBu | A-21 | H | A-21 | H |
| 44296 | 1 | H | m-D3 | H | m-D3 | H | tBu | A-22 | H | A-22 | H |
| 44297 | 1 | H | m-D3 | H | m-D3 | H | tBu | A-23 | H | A-23 | H |
| 44298 | 1 | H | m-D3 | H | m-D3 | H | tBu | A-24 | H | A-24 | H |
| 44299 | 1 | H | m-D3 | H | m-D3 | H | tBu | A-25 | H | A-25 | H |
| 44300 | 1 | H | m-D3 | H | m-D3 | H | tBu | A-26 | H | A-26 | H |
| 44301 | 1 | H | m-D3 | H | m-D3 | H | tBu | A-27 | H | A-27 | H |
| 44302 | 1 | H | m-D3 | H | m-D3 | H | tBu | A-28 | H | A-28 | H |
| 44303 | 1 | H | m-D3 | H | m-D3 | H | tBu | A-29 | H | A-29 | H |
| 44304 | 1 | H | m-D3 | H | m-D3 | H | tBu | A-30 | H | A-30 | H |
| 44305 | 1 | H | m-D3 | H | m-D3 | H | tBu | A-31 | H | A-31 | H |
| 44306 | 1 | H | m-D3 | H | m-D3 | H | tBu | A-32 | H | A-32 | H |
| 44307 | 1 | H | m-D3 | H | m-D3 | H | tBu | A-33 | H | A-33 | H |
| 44308 | 1 | H | m-D3 | H | m-D3 | H | tBu | A-34 | H | A-34 | H |
| 44309 | 1 | H | m-D3 | H | m-D3 | H | tBu | A-35 | H | A-35 | H |
| 44310 | 1 | H | m-D3 | H | m-D3 | H | tBu | A-36 | H | A-36 | H |
| 44311 | 1 | H | m-D3 | H | m-D3 | H | tBu | A-37 | H | A-37 | H |
| 44312 | 1 | H | m-D3 | H | m-D3 | H | tBu | A-38 | H | A-38 | H |
| 44313 | 1 | H | m-D3 | H | m-D3 | H | tBu | A-39 | H | A-39 | H |
| 44314 | 1 | H | m-D3 | H | m-D3 | H | tBu | A-40 | H | A-40 | H |
| 44315 | 1 | H | m-D3 | H | m-D3 | H | tBu | A-41 | H | A-41 | H |
| 44316 | 1 | H | m-D3 | H | m-D3 | H | tBu | A-42 | H | A-42 | H |
| 44317 | 1 | H | m-D3 | H | m-D3 | H | tBu | A-43 | H | A-43 | H |
| 44318 | 1 | H | m-D3 | H | m-D3 | H | tBu | A-44 | H | A-44 | H |
| 44319 | 1 | H | m-D3 | H | m-D3 | H | tBu | A-45 | H | A-45 | H |
| 44320 | 1 | H | m-D3 | H | m-D3 | H | tBu | A-46 | H | A-46 | H |
| 44321 | 1 | H | m-D3 | H | m-D3 | H | tBu | A-47 | H | A-47 | H |
| 44322 | 1 | H | m-D4 | H | m-D4 | H | tBu | A-1 | H | A-1 | H |
| 44323 | 1 | H | m-D4 | H | m-D4 | H | tBu | A-2 | H | A-2 | H |
| 44324 | 1 | H | m-D4 | H | m-D4 | H | tBu | A-3 | H | A-3 | H |
| 44325 | 1 | H | m-D4 | H | m-D4 | H | tBu | A-4 | H | A-4 | H |
| 44326 | 1 | H | m-D4 | H | m-D4 | H | tBu | A-5 | H | A-5 | H |
| 44327 | 1 | H | m-D4 | H | m-D4 | H | tBu | A-6 | H | A-6 | H |
| 44328 | 1 | H | m-D4 | H | m-D4 | H | tBu | A-7 | H | A-7 | H |
| 44329 | 1 | H | m-D4 | H | m-D4 | H | tBu | A-8 | H | A-8 | H |
| 44330 | 1 | H | m-D4 | H | m-D4 | H | tBu | A-9 | H | A-9 | H |
| 44331 | 1 | H | m-D4 | H | m-D4 | H | tBu | A-10 | H | A-10 | H |
| 44332 | 1 | H | m-D4 | H | m-D4 | H | tBu | A-11 | H | A-11 | H |
| 44333 | 1 | H | m-D4 | H | m-D4 | H | tBu | A-12 | H | A-12 | H |
| 44334 | 1 | H | m-D4 | H | m-D4 | H | tBu | A-13 | H | A-13 | H |
| 44335 | 1 | H | m-D4 | H | m-D4 | H | tBu | A-14 | H | A-14 | H |
| 44336 | 1 | H | m-D4 | H | m-D4 | H | tBu | A-15 | H | A-15 | H |
| 44337 | 1 | H | m-D4 | H | m-D4 | H | tBu | A-16 | H | A-16 | H |
| 44338 | 1 | H | m-D4 | H | m-D4 | H | tBu | A-17 | H | A-17 | H |
| 44339 | 1 | H | m-D4 | H | m-D4 | H | tBu | A-18 | H | A-18 | H |
| 44340 | 1 | H | m-D4 | H | m-D4 | H | tBu | A-19 | H | A-19 | H |
| 44341 | 1 | H | m-D4 | H | m-D4 | H | tBu | A-20 | H | A-20 | H |
| 44342 | 1 | H | m-D4 | H | m-D4 | H | tBu | A-21 | H | A-21 | H |
| 44343 | 1 | H | m-D4 | H | m-D4 | H | tBu | A-22 | H | A-22 | H |
| 44344 | 1 | H | m-D4 | H | m-D4 | H | tBu | A-23 | H | A-23 | H |
| 44345 | 1 | H | m-D4 | H | m-D4 | H | tBu | A-24 | H | A-24 | H |
| 44346 | 1 | H | m-D4 | H | m-D4 | H | tBu | A-25 | H | A-25 | H |
| 44347 | 1 | H | m-D4 | H | m-D4 | H | tBu | A-26 | H | A-26 | H |
| 44348 | 1 | H | m-D4 | H | m-D4 | H | tBu | A-27 | H | A-27 | H |
| 44349 | 1 | H | m-D4 | H | m-D4 | H | tBu | A-28 | H | A-28 | H |
| 44350 | 1 | H | m-D4 | H | m-D4 | H | tBu | A-29 | H | A-29 | H |
| 44351 | 1 | H | m-D4 | H | m-D4 | H | tBu | A-30 | H | A-30 | H |
| 44352 | 1 | H | m-D4 | H | m-D4 | H | tBu | A-31 | H | A-31 | H |
| 44353 | 1 | H | m-D4 | H | m-D4 | H | tBu | A-32 | H | A-32 | H |
| 44354 | 1 | H | m-D4 | H | m-D4 | H | tBu | A-33 | H | A-33 | H |
| 44355 | 1 | H | m-D4 | H | m-D4 | H | tBu | A-34 | H | A-34 | H |
| 44356 | 1 | H | m-D4 | H | m-D4 | H | tBu | A-35 | H | A-35 | H |
| 44357 | 1 | H | m-D4 | H | m-D4 | H | tBu | A-36 | H | A-36 | H |
| 44358 | 1 | H | m-D4 | H | m-D4 | H | tBu | A-37 | H | A-37 | H |
| 44359 | 1 | H | m-D4 | H | m-D4 | H | tBu | A-38 | H | A-38 | H |
| 44360 | 1 | H | m-D4 | H | m-D4 | H | tBu | A-39 | H | A-39 | H |
| 44361 | 1 | H | m-D4 | H | m-D4 | H | tBu | A-40 | H | A-40 | H |
| 44362 | 1 | H | m-D4 | H | m-D4 | H | tBu | A-41 | H | A-41 | H |
| 44363 | 1 | H | m-D4 | H | m-D4 | H | tBu | A-42 | H | A-42 | H |
| 44364 | 1 | H | m-D4 | H | m-D4 | H | tBu | A-43 | H | A-43 | H |
| 44365 | 1 | H | m-D4 | H | m-D4 | H | tBu | A-44 | H | A-44 | H |
| 44366 | 1 | H | m-D4 | H | m-D4 | H | tBu | A-45 | H | A-45 | H |
| 44367 | 1 | H | m-D4 | H | m-D4 | H | tBu | A-46 | H | A-46 | H |
| 44368 | 1 | H | m-D4 | H | m-D4 | H | tBu | A-47 | H | A-47 | H |
| 44369 | 1 | H | m-D5 | H | m-D5 | H | tBu | A-1 | H | A-1 | H |
| 44370 | 1 | H | m-D5 | H | m-D5 | H | tBu | A-2 | H | A-2 | H |
| 44371 | 1 | H | m-D5 | H | m-D5 | H | tBu | A-3 | H | A-3 | H |
| 44372 | 1 | H | m-D5 | H | m-D5 | H | tBu | A-4 | H | A-4 | H |
| 44373 | 1 | H | m-D5 | H | m-D5 | H | tBu | A-5 | H | A-5 | H |
| 44374 | 1 | H | m-D5 | H | m-D5 | H | tBu | A-6 | H | A-6 | H |
| 44375 | 1 | H | m-D5 | H | m-D5 | H | tBu | A-7 | H | A-7 | H |
| 44376 | 1 | H | m-D5 | H | m-D5 | H | tBu | A-8 | H | A-8 | H |
| 44377 | 1 | H | m-D5 | H | m-D5 | H | tBu | A-9 | H | A-9 | H |
| 44378 | 1 | H | m-D5 | H | m-D5 | H | tBu | A-10 | H | A-10 | H |
| 44379 | 1 | H | m-D5 | H | m-D5 | H | tBu | A-11 | H | A-11 | H |
| 44380 | 1 | H | m-D5 | H | m-D5 | H | tBu | A-12 | H | A-12 | H |
| 44381 | 1 | H | m-D5 | H | m-D5 | H | tBu | A-13 | H | A-13 | H |
| 44382 | 1 | H | m-D5 | H | m-D5 | H | tBu | A-14 | H | A-14 | H |
| 44383 | 1 | H | m-D5 | H | m-D5 | H | tBu | A-15 | H | A-15 | H |
| 44384 | 1 | H | m-D5 | H | m-D5 | H | tBu | A-16 | H | A-16 | H |
| 44385 | 1 | H | m-D5 | H | m-D5 | H | tBu | A-17 | H | A-17 | H |
| 44386 | 1 | H | m-D5 | H | m-D5 | H | tBu | A-18 | H | A-18 | H |
| 44387 | 1 | H | m-D5 | H | m-D5 | H | tBu | A-19 | H | A-19 | H |
| 44388 | 1 | H | m-D5 | H | m-D5 | H | tBu | A-20 | H | A-20 | H |
| 44389 | 1 | H | m-D5 | H | m-D5 | H | tBu | A-21 | H | A-21 | H |
| 44390 | 1 | H | m-D5 | H | m-D5 | H | tBu | A-22 | H | A-22 | H |
| 44391 | 1 | H | m-D5 | H | m-D5 | H | tBu | A-23 | H | A-23 | H |
| 44392 | 1 | H | m-D5 | H | m-D5 | H | tBu | A-24 | H | A-24 | H |
| 44393 | 1 | H | m-D5 | H | m-D5 | H | tBu | A-25 | H | A-25 | H |
| 44394 | 1 | H | m-D5 | H | m-D5 | H | tBu | A-26 | H | A-26 | H |
| 44395 | 1 | H | m-D5 | H | m-D5 | H | tBu | A-27 | H | A-27 | H |
| 44396 | 1 | H | m-D5 | H | m-D5 | H | tBu | A-28 | H | A-28 | H |
| 44397 | 1 | H | m-D5 | H | m-D5 | H | tBu | A-29 | H | A-29 | H |
| 44398 | 1 | H | m-D5 | H | m-D5 | H | tBu | A-30 | H | A-30 | H |
| 44399 | 1 | H | m-D5 | H | m-D5 | H | tBu | A-31 | H | A-31 | H |
| 44400 | 1 | H | m-D5 | H | m-D5 | H | tBu | A-32 | H | A-32 | H |
| 44401 | 1 | H | m-D5 | H | m-D5 | H | tBu | A-33 | H | A-33 | H |
| 44402 | 1 | H | m-D5 | H | m-D5 | H | tBu | A-34 | H | A-34 | H |
| 44403 | 1 | H | m-D5 | H | m-D5 | H | tBu | A-35 | H | A-35 | H |
| 44404 | 1 | H | m-D5 | H | m-D5 | H | tBu | A-36 | H | A-36 | H |
| 44405 | 1 | H | m-D5 | H | m-D5 | H | tBu | A-37 | H | A-37 | H |
| 44406 | 1 | H | m-D5 | H | m-D5 | H | tBu | A-38 | H | A-38 | H |
| 44407 | 1 | H | m-D5 | H | m-D5 | H | tBu | A-39 | H | A-39 | H |
| 44408 | 1 | H | m-D5 | H | m-D5 | H | tBu | A-40 | H | A-40 | H |
| 44409 | 1 | H | m-D5 | H | m-D5 | H | tBu | A-41 | H | A-41 | H |
| 44410 | 1 | H | m-D5 | H | m-D5 | H | tBu | A-42 | H | A-42 | H |
| 44411 | 1 | H | m-D5 | H | m-D5 | H | tBu | A-43 | H | A-43 | H |
| 44412 | 1 | H | m-D5 | H | m-D5 | H | tBu | A-44 | H | A-44 | H |
| 44413 | 1 | H | m-D5 | H | m-D5 | H | tBu | A-45 | H | A-45 | H |
| 44414 | 1 | H | m-D5 | H | m-D5 | H | tBu | A-46 | H | A-46 | H |
| 44415 | 1 | H | m-D5 | H | m-D5 | H | tBu | A-47 | H | A-47 | H |
| 44416 | 1 | H | m-D6 | H | m-D6 | H | tBu | A-1 | H | A-1 | H |
| 44417 | 1 | H | m-D6 | H | m-D6 | H | tBu | A-2 | H | A-2 | H |
| 44418 | 1 | H | m-D6 | H | m-D6 | H | tBu | A-3 | H | A-3 | H |
| 44419 | 1 | H | m-D6 | H | m-D6 | H | tBu | A-4 | H | A-4 | H |
| 44420 | 1 | H | m-D6 | H | m-D6 | H | tBu | A-5 | H | A-5 | H |
| 44421 | 1 | H | m-D6 | H | m-D6 | H | tBu | A-6 | H | A-6 | H |
| 44422 | 1 | H | m-D6 | H | m-D6 | H | tBu | A-7 | H | A-7 | H |
| 44423 | 1 | H | m-D6 | H | m-D6 | H | tBu | A-8 | H | A-8 | H |
| 44424 | 1 | H | m-D6 | H | m-D6 | H | tBu | A-9 | H | A-9 | H |
| 44425 | 1 | H | m-D6 | H | m-D6 | H | tBu | A-10 | H | A-10 | H |
| 44426 | 1 | H | m-D6 | H | m-D6 | H | tBu | A-11 | H | A-11 | H |
| 44427 | 1 | H | m-D6 | H | m-D6 | H | tBu | A-12 | H | A-12 | H |
| 44428 | 1 | H | m-D6 | H | m-D6 | H | tBu | A-13 | H | A-13 | H |
| 44429 | 1 | H | m-D6 | H | m-D6 | H | tBu | A-14 | H | A-14 | H |
| 44430 | 1 | H | m-D6 | H | m-D6 | H | tBu | A-15 | H | A-15 | H |
| 44431 | 1 | H | m-D6 | H | m-D6 | H | tBu | A-16 | H | A-16 | H |
| 44432 | 1 | H | m-D6 | H | m-D6 | H | tBu | A-17 | H | A-17 | H |
| 44433 | 1 | H | m-D6 | H | m-D6 | H | tBu | A-18 | H | A-18 | H |
| 44434 | 1 | H | m-D6 | H | m-D6 | H | tBu | A-19 | H | A-19 | H |
| 44435 | 1 | H | m-D6 | H | m-D6 | H | tBu | A-20 | H | A-20 | H |
| 44436 | 1 | H | m-D6 | H | m-D6 | H | tBu | A-21 | H | A-21 | H |
| 44437 | 1 | H | m-D6 | H | m-D6 | H | tBu | A-22 | H | A-22 | H |
| 44438 | 1 | H | m-D6 | H | m-D6 | H | tBu | A-23 | H | A-23 | H |
| 44439 | 1 | H | m-D6 | H | m-D6 | H | tBu | A-24 | H | A-24 | H |
| 44440 | 1 | H | m-D6 | H | m-D6 | H | tBu | A-25 | H | A-25 | H |
| 44441 | 1 | H | m-D6 | H | m-D6 | H | tBu | A-26 | H | A-26 | H |
| 44442 | 1 | H | m-D6 | H | m-D6 | H | tBu | A-27 | H | A-27 | H |
| 44443 | 1 | H | m-D6 | H | m-D6 | H | tBu | A-28 | H | A-28 | H |
| 44444 | 1 | H | m-D6 | H | m-D6 | H | tBu | A-29 | H | A-29 | H |
| 44445 | 1 | H | m-D6 | H | m-D6 | H | tBu | A-30 | H | A-30 | H |
| 44446 | 1 | H | m-D6 | H | m-D6 | H | tBu | A-31 | H | A-31 | H |
| 44447 | 1 | H | m-D6 | H | m-D6 | H | tBu | A-32 | H | A-32 | H |
| 44448 | 1 | H | m-D6 | H | m-D6 | H | tBu | A-33 | H | A-33 | H |
| 44449 | 1 | H | m-D6 | H | m-D6 | H | tBu | A-34 | H | A-34 | H |
| 44450 | 1 | H | m-D6 | H | m-D6 | H | tBu | A-35 | H | A-35 | H |
| 44451 | 1 | H | m-D6 | H | m-D6 | H | tBu | A-36 | H | A-36 | H |
| 44452 | 1 | H | m-D6 | H | m-D6 | H | tBu | A-37 | H | A-37 | H |
| 44453 | 1 | H | m-D6 | H | m-D6 | H | tBu | A-38 | H | A-38 | H |
| 44454 | 1 | H | m-D6 | H | m-D6 | H | tBu | A-39 | H | A-39 | H |
| 44455 | 1 | H | m-D6 | H | m-D6 | H | tBu | A-40 | H | A-40 | H |
| 44456 | 1 | H | m-D6 | H | m-D6 | H | tBu | A-41 | H | A-41 | H |
| 44457 | 1 | H | m-D6 | H | m-D6 | H | tBu | A-42 | H | A-42 | H |
| 44458 | 1 | H | m-D6 | H | m-D6 | H | tBu | A-43 | H | A-43 | H |
| 44459 | 1 | H | m-D6 | H | m-D6 | H | tBu | A-44 | H | A-44 | H |
| 44460 | 1 | H | m-D6 | H | m-D6 | H | tBu | A-45 | H | A-45 | H |
| 44461 | 1 | H | m-D6 | H | m-D6 | H | tBu | A-46 | H | A-46 | H |
| 44462 | 1 | H | m-D6 | H | m-D6 | H | tBu | A-47 | H | A-47 | H |
| 44463 | 1 | H | m-D7 | H | m-D7 | H | tBu | A-1 | H | A-1 | H |
| 44464 | 1 | H | m-D7 | H | m-D7 | H | tBu | A-2 | H | A-2 | H |
| 44465 | 1 | H | m-D7 | H | m-D7 | H | tBu | A-3 | H | A-3 | H |
| 44466 | 1 | H | m-D7 | H | m-D7 | H | tBu | A-4 | H | A-4 | H |
| 44467 | 1 | H | m-D7 | H | m-D7 | H | tBu | A-5 | H | A-5 | H |
| 44468 | 1 | H | m-D7 | H | m-D7 | H | tBu | A-6 | H | A-6 | H |
| 44469 | 1 | H | m-D7 | H | m-D7 | H | tBu | A-7 | H | A-7 | H |
| 44470 | 1 | H | m-D7 | H | m-D7 | H | tBu | A-8 | H | A-8 | H |
| 44471 | 1 | H | m-D7 | H | m-D7 | H | tBu | A-9 | H | A-9 | H |
| 44472 | 1 | H | m-D7 | H | m-D7 | H | tBu | A-10 | H | A-10 | H |
| 44473 | 1 | H | m-D7 | H | m-D7 | H | tBu | A-11 | H | A-11 | H |
| 44474 | 1 | H | m-D7 | H | m-D7 | H | tBu | A-12 | H | A-12 | H |
| 44475 | 1 | H | m-D7 | H | m-D7 | H | tBu | A-13 | H | A-13 | H |
| 44476 | 1 | H | m-D7 | H | m-D7 | H | tBu | A-14 | H | A-14 | H |
| 44477 | 1 | H | m-D7 | H | m-D7 | H | tBu | A-15 | H | A-15 | H |
| 44478 | 1 | H | m-D7 | H | m-D7 | H | tBu | A-16 | H | A-16 | H |
| 44479 | 1 | H | m-D7 | H | m-D7 | H | tBu | A-17 | H | A-17 | H |
| 44480 | 1 | H | m-D7 | H | m-D7 | H | tBu | A-18 | H | A-18 | H |
| 44481 | 1 | H | m-D7 | H | m-D7 | H | tBu | A-19 | H | A-19 | H |
| 44482 | 1 | H | m-D7 | H | m-D7 | H | tBu | A-20 | H | A-20 | H |
| 44483 | 1 | H | m-D7 | H | m-D7 | H | tBu | A-21 | H | A-21 | H |
| 44484 | 1 | H | m-D7 | H | m-D7 | H | tBu | A-22 | H | A-22 | H |
| 44485 | 1 | H | m-D7 | H | m-D7 | H | tBu | A-23 | H | A-23 | H |
| 44486 | 1 | H | m-D7 | H | m-D7 | H | tBu | A-24 | H | A-24 | H |
| 44487 | 1 | H | m-D7 | H | m-D7 | H | tBu | A-25 | H | A-25 | H |
| 44488 | 1 | H | m-D7 | H | m-D7 | H | tBu | A-26 | H | A-26 | H |
| 44489 | 1 | H | m-D7 | H | m-D7 | H | tBu | A-27 | H | A-27 | H |
| 44490 | 1 | H | m-D7 | H | m-D7 | H | tBu | A-28 | H | A-28 | H |
| 44491 | 1 | H | m-D7 | H | m-D7 | H | tBu | A-29 | H | A-29 | H |
| 44492 | 1 | H | m-D7 | H | m-D7 | H | tBu | A-30 | H | A-30 | H |
| 44493 | 1 | H | m-D7 | H | m-D7 | H | tBu | A-31 | H | A-31 | H |
| 44494 | 1 | H | m-D7 | H | m-D7 | H | tBu | A-32 | H | A-32 | H |
| 44495 | 1 | H | m-D7 | H | m-D7 | H | tBu | A-33 | H | A-33 | H |
| 44496 | 1 | H | m-D7 | H | m-D7 | H | tBu | A-34 | H | A-34 | H |
| 44497 | 1 | H | m-D7 | H | m-D7 | H | tBu | A-35 | H | A-35 | H |
| 44498 | 1 | H | m-D7 | H | m-D7 | H | tBu | A-36 | H | A-36 | H |
| 44499 | 1 | H | m-D7 | H | m-D7 | H | tBu | A-37 | H | A-37 | H |
| 44500 | 1 | H | m-D7 | H | m-D7 | H | tBu | A-38 | H | A-38 | H |
| 44501 | 1 | H | m-D7 | H | m-D7 | H | tBu | A-39 | H | A-39 | H |
| 44502 | 1 | H | m-D7 | H | m-D7 | H | tBu | A-40 | H | A-40 | H |
| 44503 | 1 | H | m-D7 | H | m-D7 | H | tBu | A-41 | H | A-41 | H |
| 44504 | 1 | H | m-D7 | H | m-D7 | H | tBu | A-42 | H | A-42 | H |
| 44505 | 1 | H | m-D7 | H | m-D7 | H | tBu | A-43 | H | A-43 | H |
| 44506 | 1 | H | m-D7 | H | m-D7 | H | tBu | A-44 | H | A-44 | H |
| 44507 | 1 | H | m-D7 | H | m-D7 | H | tBu | A-45 | H | A-45 | H |
| 44508 | 1 | H | m-D7 | H | m-D7 | H | tBu | A-46 | H | A-46 | H |
| 44509 | 1 | H | m-D7 | H | m-D7 | H | tBu | A-47 | H | A-47 | H |
| 44510 | 1 | H | m-D8 | H | m-D8 | H | tBu | A-1 | H | A-1 | H |
| 44511 | 1 | H | m-D8 | H | m-D8 | H | tBu | A-2 | H | A-2 | H |
| 44512 | 1 | H | m-D8 | H | m-D8 | H | tBu | A-3 | H | A-3 | H |
| 44513 | 1 | H | m-D8 | H | m-D8 | H | tBu | A-4 | H | A-4 | H |
| 44514 | 1 | H | m-D8 | H | m-D8 | H | tBu | A-5 | H | A-5 | H |
| 44515 | 1 | H | m-D8 | H | m-D8 | H | tBu | A-6 | H | A-6 | H |
| 44516 | 1 | H | m-D8 | H | m-D8 | H | tBu | A-7 | H | A-7 | H |
| 44517 | 1 | H | m-D8 | H | m-D8 | H | tBu | A-8 | H | A-8 | H |
| 44518 | 1 | H | m-D8 | H | m-D8 | H | tBu | A-9 | H | A-9 | H |
| 44519 | 1 | H | m-D8 | H | m-D8 | H | tBu | A-10 | H | A-10 | H |
| 44520 | 1 | H | m-D8 | H | m-D8 | H | tBu | A-11 | H | A-11 | H |
| 44521 | 1 | H | m-D8 | H | m-D8 | H | tBu | A-12 | H | A-12 | H |
| 44522 | 1 | H | m-D8 | H | m-D8 | H | tBu | A-13 | H | A-13 | H |
| 44523 | 1 | H | m-D8 | H | m-D8 | H | tBu | A-14 | H | A-14 | H |
| 44524 | 1 | H | m-D8 | H | m-D8 | H | tBu | A-15 | H | A-15 | H |
| 44525 | 1 | H | m-D8 | H | m-D8 | H | tBu | A-16 | H | A-16 | H |
| 44526 | 1 | H | m-D8 | H | m-D8 | H | tBu | A-17 | H | A-17 | H |
| 44527 | 1 | H | m-D8 | H | m-D8 | H | tBu | A-18 | H | A-18 | H |
| 44528 | 1 | H | m-D8 | H | m-D8 | H | tBu | A-19 | H | A-19 | H |
| 44529 | 1 | H | m-D8 | H | m-D8 | H | tBu | A-20 | H | A-20 | H |
| 44530 | 1 | H | m-D8 | H | m-D8 | H | tBu | A-21 | H | A-21 | H |
| 44531 | 1 | H | m-D8 | H | m-D8 | H | tBu | A-22 | H | A-22 | H |
| 44532 | 1 | H | m-D8 | H | m-D8 | H | tBu | A-23 | H | A-23 | H |
| 44533 | 1 | H | m-D8 | H | m-D8 | H | tBu | A-24 | H | A-24 | H |
| 44534 | 1 | H | m-D8 | H | m-D8 | H | tBu | A-25 | H | A-25 | H |
| 44535 | 1 | H | m-D8 | H | m-D8 | H | tBu | A-26 | H | A-26 | H |
| 44536 | 1 | H | m-D8 | H | m-D8 | H | tBu | A-27 | H | A-27 | H |
| 44537 | 1 | H | m-D8 | H | m-D8 | H | tBu | A-28 | H | A-28 | H |
| 44538 | 1 | H | m-D8 | H | m-D8 | H | tBu | A-29 | H | A-29 | H |
| 44539 | 1 | H | m-D8 | H | m-D8 | H | tBu | A-30 | H | A-30 | H |
| 44540 | 1 | H | m-D8 | H | m-D8 | H | tBu | A-31 | H | A-31 | H |
| 44541 | 1 | H | m-D8 | H | m-D8 | H | tBu | A-32 | H | A-32 | H |
| 44542 | 1 | H | m-D8 | H | m-D8 | H | tBu | A-33 | H | A-33 | H |
| 44543 | 1 | H | m-D8 | H | m-D8 | H | tBu | A-34 | H | A-34 | H |
| 44544 | 1 | H | m-D8 | H | m-D8 | H | tBu | A-35 | H | A-35 | H |
| 44545 | 1 | H | m-D8 | H | m-D8 | H | tBu | A-36 | H | A-36 | H |
| 44546 | 1 | H | m-D8 | H | m-D8 | H | tBu | A-37 | H | A-37 | H |
| 44547 | 1 | H | m-D8 | H | m-D8 | H | tBu | A-38 | H | A-38 | H |
| 44548 | 1 | H | m-D8 | H | m-D8 | H | tBu | A-39 | H | A-39 | H |
| 44549 | 1 | H | m-D8 | H | m-D8 | H | tBu | A-40 | H | A-40 | H |
| 44550 | 1 | H | m-D8 | H | m-D8 | H | tBu | A-41 | H | A-41 | H |
| 44551 | 1 | H | m-D8 | H | m-D8 | H | tBu | A-42 | H | A-42 | H |
| 44552 | 1 | H | m-D8 | H | m-D8 | H | tBu | A-43 | H | A-43 | H |
| 44553 | 1 | H | m-D8 | H | m-D8 | H | tBu | A-44 | H | A-44 | H |
| 44554 | 1 | H | m-D8 | H | m-D8 | H | tBu | A-45 | H | A-45 | H |
| 44555 | 1 | H | m-D8 | H | m-D8 | H | tBu | A-46 | H | A-46 | H |
| 44556 | 1 | H | m-D8 | H | m-D8 | H | tBu | A-47 | H | A-47 | H |
| 44557 | 1 | H | m-D9 | H | m-D9 | H | tBu | A-1 | H | A-1 | H |
| 44558 | 1 | H | m-D9 | H | m-D9 | H | tBu | A-2 | H | A-2 | H |
| 44559 | 1 | H | m-D9 | H | m-D9 | H | tBu | A-3 | H | A-3 | H |
| 44560 | 1 | H | m-D9 | H | m-D9 | H | tBu | A-4 | H | A-4 | H |
| 44561 | 1 | H | m-D9 | H | m-D9 | H | tBu | A-5 | H | A-5 | H |
| 44562 | 1 | H | m-D9 | H | m-D9 | H | tBu | A-6 | H | A-6 | H |
| 44563 | 1 | H | m-D9 | H | m-D9 | H | tBu | A-7 | H | A-7 | H |
| 44564 | 1 | H | m-D9 | H | m-D9 | H | tBu | A-8 | H | A-8 | H |
| 44565 | 1 | H | m-D9 | H | m-D9 | H | tBu | A-9 | H | A-9 | H |
| 44566 | 1 | H | m-D9 | H | m-D9 | H | tBu | A-10 | H | A-10 | H |
| 44567 | 1 | H | m-D9 | H | m-D9 | H | tBu | A-11 | H | A-11 | H |
| 44568 | 1 | H | m-D9 | H | m-D9 | H | tBu | A-12 | H | A-12 | H |
| 44569 | 1 | H | m-D9 | H | m-D9 | H | tBu | A-13 | H | A-13 | H |
| 44570 | 1 | H | m-D9 | H | m-D9 | H | tBu | A-14 | H | A-14 | H |
| 44571 | 1 | H | m-D9 | H | m-D9 | H | tBu | A-15 | H | A-15 | H |
| 44572 | 1 | H | m-D9 | H | m-D9 | H | tBu | A-16 | H | A-16 | H |
| 44573 | 1 | H | m-D9 | H | m-D9 | H | tBu | A-17 | H | A-17 | H |
| 44574 | 1 | H | m-D9 | H | m-D9 | H | tBu | A-18 | H | A-18 | H |
| 44575 | 1 | H | m-D9 | H | m-D9 | H | tBu | A-19 | H | A-19 | H |
| 44576 | 1 | H | m-D9 | H | m-D9 | H | tBu | A-20 | H | A-20 | H |
| 44577 | 1 | H | m-D9 | H | m-D9 | H | tBu | A-21 | H | A-21 | H |
| 44578 | 1 | H | m-D9 | H | m-D9 | H | tBu | A-22 | H | A-22 | H |
| 44579 | 1 | H | m-D9 | H | m-D9 | H | tBu | A-23 | H | A-23 | H |
| 44580 | 1 | H | m-D9 | H | m-D9 | H | tBu | A-24 | H | A-24 | H |
| 44581 | 1 | H | m-D9 | H | m-D9 | H | tBu | A-25 | H | A-25 | H |
| 44582 | 1 | H | m-D9 | H | m-D9 | H | tBu | A-26 | H | A-26 | H |
| 44583 | 1 | H | m-D9 | H | m-D9 | H | tBu | A-27 | H | A-27 | H |
| 44584 | 1 | H | m-D9 | H | m-D9 | H | tBu | A-28 | H | A-28 | H |
| 44585 | 1 | H | m-D9 | H | m-D9 | H | tBu | A-29 | H | A-29 | H |
| 44586 | 1 | H | m-D9 | H | m-D9 | H | tBu | A-30 | H | A-30 | H |
| 44587 | 1 | H | m-D9 | H | m-D9 | H | tBu | A-31 | H | A-31 | H |
| 44588 | 1 | H | m-D9 | H | m-D9 | H | tBu | A-32 | H | A-32 | H |
| 44589 | 1 | H | m-D9 | H | m-D9 | H | tBu | A-33 | H | A-33 | H |
| 44590 | 1 | H | m-D9 | H | m-D9 | H | tBu | A-34 | H | A-34 | H |
| 44591 | 1 | H | m-D9 | H | m-D9 | H | tBu | A-35 | H | A-35 | H |
| 44592 | 1 | H | m-D9 | H | m-D9 | H | tBu | A-36 | H | A-36 | H |
| 44593 | 1 | H | m-D9 | H | m-D9 | H | tBu | A-37 | H | A-37 | H |
| 44594 | 1 | H | m-D9 | H | m-D9 | H | tBu | A-38 | H | A-38 | H |
| 44595 | 1 | H | m-D9 | H | m-D9 | H | tBu | A-39 | H | A-39 | H |
| 44596 | 1 | H | m-D9 | H | m-D9 | H | tBu | A-40 | H | A-40 | H |
| 44597 | 1 | H | m-D9 | H | m-D9 | H | tBu | A-41 | H | A-41 | H |
| 44598 | 1 | H | m-D9 | H | m-D9 | H | tBu | A-42 | H | A-42 | H |
| 44599 | 1 | H | m-D9 | H | m-D9 | H | tBu | A-43 | H | A-43 | H |
| 44600 | 1 | H | m-D9 | H | m-D9 | H | tBu | A-44 | H | A-44 | H |
| 44601 | 1 | H | m-D9 | H | m-D9 | H | tBu | A-45 | H | A-45 | H |
| 44602 | 1 | H | m-D9 | H | m-D9 | H | tBu | A-46 | H | A-46 | H |
| 44603 | 1 | H | m-D9 | H | m-D9 | H | tBu | A-47 | H | A-47 | H |
| 44604 | 1 | H | m-D10 | H | m-D10 | H | tBu | A-1 | H | A-1 | H |
| 44605 | 1 | H | m-D10 | H | m-D10 | H | tBu | A-2 | H | A-2 | H |
| 44606 | 1 | H | m-D10 | H | m-D10 | H | tBu | A-3 | H | A-3 | H |
| 44607 | 1 | H | m-D10 | H | m-D10 | H | tBu | A-4 | H | A-4 | H |
| 44608 | 1 | H | m-D10 | H | m-D10 | H | tBu | A-5 | H | A-5 | H |
| 44609 | 1 | H | m-D10 | H | m-D10 | H | tBu | A-6 | H | A-6 | H |
| 44610 | 1 | H | m-D10 | H | m-D10 | H | tBu | A-7 | H | A-7 | H |
| 44611 | 1 | H | m-D10 | H | m-D10 | H | tBu | A-8 | H | A-8 | H |
| 44612 | 1 | H | m-D10 | H | m-D10 | H | tBu | A-9 | H | A-9 | H |
| 44613 | 1 | H | m-D10 | H | m-D10 | H | tBu | A-10 | H | A-10 | H |
| 44614 | 1 | H | m-D10 | H | m-D10 | H | tBu | A-11 | H | A-11 | H |
| 44615 | 1 | H | m-D10 | H | m-D10 | H | tBu | A-12 | H | A-12 | H |
| 44616 | 1 | H | m-D10 | H | m-D10 | H | tBu | A-13 | H | A-13 | H |
| 44617 | 1 | H | m-D10 | H | m-D10 | H | tBu | A-14 | H | A-14 | H |
| 44618 | 1 | H | m-D10 | H | m-D10 | H | tBu | A-15 | H | A-15 | H |
| 44619 | 1 | H | m-D10 | H | m-D10 | H | tBu | A-16 | H | A-16 | H |
| 44620 | 1 | H | m-D10 | H | m-D10 | H | tBu | A-17 | H | A-17 | H |
| 44621 | 1 | H | m-D10 | H | m-D10 | H | tBu | A-18 | H | A-18 | H |
| 44622 | 1 | H | m-D10 | H | m-D10 | H | tBu | A-19 | H | A-19 | H |
| 44623 | 1 | H | m-D10 | H | m-D10 | H | tBu | A-20 | H | A-20 | H |
| 44624 | 1 | H | m-D10 | H | m-D10 | H | tBu | A-21 | H | A-21 | H |
| 44625 | 1 | H | m-D10 | H | m-D10 | H | tBu | A-22 | H | A-22 | H |
| 44626 | 1 | H | m-D10 | H | m-D10 | H | tBu | A-23 | H | A-23 | H |
| 44627 | 1 | H | m-D10 | H | m-D10 | H | tBu | A-24 | H | A-24 | H |
| 44628 | 1 | H | m-D10 | H | m-D10 | H | tBu | A-25 | H | A-25 | H |
| 44629 | 1 | H | m-D10 | H | m-D10 | H | tBu | A-26 | H | A-26 | H |
| 44630 | 1 | H | m-D10 | H | m-D10 | H | tBu | A-27 | H | A-27 | H |
| 44631 | 1 | H | m-D10 | H | m-D10 | H | tBu | A-28 | H | A-28 | H |
| 44632 | 1 | H | m-D10 | H | m-D10 | H | tBu | A-29 | H | A-29 | H |
| 44633 | 1 | H | m-D10 | H | m-D10 | H | tBu | A-30 | H | A-30 | H |
| 44634 | 1 | H | m-D10 | H | m-D10 | H | tBu | A-31 | H | A-31 | H |
| 44635 | 1 | H | m-D10 | H | m-D10 | H | tBu | A-32 | H | A-32 | H |
| 44636 | 1 | H | m-D10 | H | m-D10 | H | tBu | A-33 | H | A-33 | H |
| 44637 | 1 | H | m-D10 | H | m-D10 | H | tBu | A-34 | H | A-34 | H |
| 44638 | 1 | H | m-D10 | H | m-D10 | H | tBu | A-35 | H | A-35 | H |
| 44639 | 1 | H | m-D10 | H | m-D10 | H | tBu | A-36 | H | A-36 | H |
| 44640 | 1 | H | m-D10 | H | m-D10 | H | tBu | A-37 | H | A-37 | H |
| 44641 | 1 | H | m-D10 | H | m-D10 | H | tBu | A-38 | H | A-38 | H |
| 44642 | 1 | H | m-D10 | H | m-D10 | H | tBu | A-39 | H | A-39 | H |
| 44643 | 1 | H | m-D10 | H | m-D10 | H | tBu | A-40 | H | A-40 | H |
| 44644 | 1 | H | m-D10 | H | m-D10 | H | tBu | A-41 | H | A-41 | H |
| 44645 | 1 | H | m-D10 | H | m-D10 | H | tBu | A-42 | H | A-42 | H |
| 44646 | 1 | H | m-D10 | H | m-D10 | H | tBu | A-43 | H | A-43 | H |
| 44647 | 1 | H | m-D10 | H | m-D10 | H | tBu | A-44 | H | A-44 | H |
| 44648 | 1 | H | m-D10 | H | m-D10 | H | tBu | A-45 | H | A-45 | H |
| 44649 | 1 | H | m-D10 | H | m-D10 | H | tBu | A-46 | H | A-46 | H |
| 44650 | 1 | H | m-D10 | H | m-D10 | H | tBu | A-47 | H | A-47 | H |
| 44651 | 1 | H | m-D11 | H | m-D11 | H | tBu | A-1 | H | A-1 | H |
| 44652 | 1 | H | m-D11 | H | m-D11 | H | tBu | A-2 | H | A-2 | H |
| 44653 | 1 | H | m-D11 | H | m-D11 | H | tBu | A-3 | H | A-3 | H |
| 44654 | 1 | H | m-D11 | H | m-D11 | H | tBu | A-4 | H | A-4 | H |
| 44655 | 1 | H | m-D11 | H | m-D11 | H | tBu | A-5 | H | A-5 | H |
| 44656 | 1 | H | m-D11 | H | m-D11 | H | tBu | A-6 | H | A-6 | H |
| 44657 | 1 | H | m-D11 | H | m-D11 | H | tBu | A-7 | H | A-7 | H |
| 44658 | 1 | H | m-D11 | H | m-D11 | H | tBu | A-8 | H | A-8 | H |
| 44659 | 1 | H | m-D11 | H | m-D11 | H | tBu | A-9 | H | A-9 | H |
| 44660 | 1 | H | m-D11 | H | m-D11 | H | tBu | A-10 | H | A-10 | H |
| 44661 | 1 | H | m-D11 | H | m-D11 | H | tBu | A-11 | H | A-11 | H |
| 44662 | 1 | H | m-D11 | H | m-D11 | H | tBu | A-12 | H | A-12 | H |
| 44663 | 1 | H | m-D11 | H | m-D11 | H | tBu | A-13 | H | A-13 | H |
| 44664 | 1 | H | m-D11 | H | m-D11 | H | tBu | A-14 | H | A-14 | H |
| 44665 | 1 | H | m-D11 | H | m-D11 | H | tBu | A-15 | H | A-15 | H |
| 44666 | 1 | H | m-D11 | H | m-D11 | H | tBu | A-16 | H | A-16 | H |
| 44667 | 1 | H | m-D11 | H | m-D11 | H | tBu | A-17 | H | A-17 | H |
| 44668 | 1 | H | m-D11 | H | m-D11 | H | tBu | A-18 | H | A-18 | H |
| 44669 | 1 | H | m-D11 | H | m-D11 | H | tBu | A-19 | H | A-19 | H |
| 44670 | 1 | H | m-D11 | H | m-D11 | H | tBu | A-20 | H | A-20 | H |
| 44671 | 1 | H | m-D11 | H | m-D11 | H | tBu | A-21 | H | A-21 | H |
| 44672 | 1 | H | m-D11 | H | m-D11 | H | tBu | A-22 | H | A-22 | H |
| 44673 | 1 | H | m-D11 | H | m-D11 | H | tBu | A-23 | H | A-23 | H |
| 44674 | 1 | H | m-D11 | H | m-D11 | H | tBu | A-24 | H | A-24 | H |
| 44675 | 1 | H | m-D11 | H | m-D11 | H | tBu | A-25 | H | A-25 | H |
| 44676 | 1 | H | m-D11 | H | m-D11 | H | tBu | A-26 | H | A-26 | H |
| 44677 | 1 | H | m-D11 | H | m-D11 | H | tBu | A-27 | H | A-27 | H |
| 44678 | 1 | H | m-D11 | H | m-D11 | H | tBu | A-28 | H | A-28 | H |
| 44679 | 1 | H | m-D11 | H | m-D11 | H | tBu | A-29 | H | A-29 | H |
| 44680 | 1 | H | m-D11 | H | m-D11 | H | tBu | A-30 | H | A-30 | H |
| 44681 | 1 | H | m-D11 | H | m-D11 | H | tBu | A-31 | H | A-31 | H |
| 44682 | 1 | H | m-D11 | H | m-D11 | H | tBu | A-32 | H | A-32 | H |
| 44683 | 1 | H | m-D11 | H | m-D11 | H | tBu | A-33 | H | A-33 | H |
| 44684 | 1 | H | m-D11 | H | m-D11 | H | tBu | A-34 | H | A-34 | H |
| 44685 | 1 | H | m-D11 | H | m-D11 | H | tBu | A-35 | H | A-35 | H |
| 44686 | 1 | H | m-D11 | H | m-D11 | H | tBu | A-36 | H | A-36 | H |
| 44687 | 1 | H | m-D11 | H | m-D11 | H | tBu | A-37 | H | A-37 | H |
| 44688 | 1 | H | m-D11 | H | m-D11 | H | tBu | A-38 | H | A-38 | H |
| 44689 | 1 | H | m-D11 | H | m-D11 | H | tBu | A-39 | H | A-39 | H |
| 44690 | 1 | H | m-D11 | H | m-D11 | H | tBu | A-40 | H | A-40 | H |
| 44691 | 1 | H | m-D11 | H | m-D11 | H | tBu | A-41 | H | A-41 | H |
| 44692 | 1 | H | m-D11 | H | m-D11 | H | tBu | A-42 | H | A-42 | H |
| 44693 | 1 | H | m-D11 | H | m-D11 | H | tBu | A-43 | H | A-43 | H |
| 44694 | 1 | H | m-D11 | H | m-D11 | H | tBu | A-44 | H | A-44 | H |
| 44695 | 1 | H | m-D11 | H | m-D11 | H | tBu | A-45 | H | A-45 | H |
| 44696 | 1 | H | m-D11 | H | m-D11 | H | tBu | A-46 | H | A-46 | H |
| 44697 | 1 | H | m-D11 | H | m-D11 | H | tBu | A-47 | H | A-47 | H |
| 44698 | 1 | H | m-D12 | H | m-D12 | H | tBu | A-1 | H | A-1 | H |
| 44699 | 1 | H | m-D12 | H | m-D12 | H | tBu | A-2 | H | A-2 | H |
| 44700 | 1 | H | m-D12 | H | m-D12 | H | tBu | A-3 | H | A-3 | H |
| 44701 | 1 | H | m-D12 | H | m-D12 | H | tBu | A-4 | H | A-4 | H |
| 44702 | 1 | H | m-D12 | H | m-D12 | H | tBu | A-5 | H | A-5 | H |
| 44703 | 1 | H | m-D12 | H | m-D12 | H | tBu | A-6 | H | A-6 | H |
| 44704 | 1 | H | m-D12 | H | m-D12 | H | tBu | A-7 | H | A-7 | H |
| 44705 | 1 | H | m-D12 | H | m-D12 | H | tBu | A-8 | H | A-8 | H |
| 44706 | 1 | H | m-D12 | H | m-D12 | H | tBu | A-9 | H | A-9 | H |
| 44707 | 1 | H | m-D12 | H | m-D12 | H | tBu | A-10 | H | A-10 | H |
| 44708 | 1 | H | m-D12 | H | m-D12 | H | tBu | A-11 | H | A-11 | H |
| 44709 | 1 | H | m-D12 | H | m-D12 | H | tBu | A-12 | H | A-12 | H |
| 44710 | 1 | H | m-D12 | H | m-D12 | H | tBu | A-13 | H | A-13 | H |
| 44711 | 1 | H | m-D12 | H | m-D12 | H | tBu | A-14 | H | A-14 | H |
| 44712 | 1 | H | m-D12 | H | m-D12 | H | tBu | A-15 | H | A-15 | H |
| 44713 | 1 | H | m-D12 | H | m-D12 | H | tBu | A-16 | H | A-16 | H |
| 44714 | 1 | H | m-D12 | H | m-D12 | H | tBu | A-17 | H | A-17 | H |
| 44715 | 1 | H | m-D12 | H | m-D12 | H | tBu | A-18 | H | A-18 | H |
| 44716 | 1 | H | m-D12 | H | m-D12 | H | tBu | A-19 | H | A-19 | H |
| 44717 | 1 | H | m-D12 | H | m-D12 | H | tBu | A-20 | H | A-20 | H |
| 44718 | 1 | H | m-D12 | H | m-D12 | H | tBu | A-21 | H | A-21 | H |
| 44719 | 1 | H | m-D12 | H | m-D12 | H | tBu | A-22 | H | A-22 | H |
| 44720 | 1 | H | m-D12 | H | m-D12 | H | tBu | A-23 | H | A-23 | H |
| 44721 | 1 | H | m-D12 | H | m-D12 | H | tBu | A-24 | H | A-24 | H |
| 44722 | 1 | H | m-D12 | H | m-D12 | H | tBu | A-25 | H | A-25 | H |
| 44723 | 1 | H | m-D12 | H | m-D12 | H | tBu | A-26 | H | A-26 | H |
| 44724 | 1 | H | m-D12 | H | m-D12 | H | tBu | A-27 | H | A-27 | H |
| 44725 | 1 | H | m-D12 | H | m-D12 | H | tBu | A-28 | H | A-28 | H |
| 44726 | 1 | H | m-D12 | H | m-D12 | H | tBu | A-29 | H | A-29 | H |
| 44727 | 1 | H | m-D12 | H | m-D12 | H | tBu | A-30 | H | A-30 | H |
| 44728 | 1 | H | m-D12 | H | m-D12 | H | tBu | A-31 | H | A-31 | H |
| 44729 | 1 | H | m-D12 | H | m-D12 | H | tBu | A-32 | H | A-32 | H |
| 44730 | 1 | H | m-D12 | H | m-D12 | H | tBu | A-33 | H | A-33 | H |
| 44731 | 1 | H | m-D12 | H | m-D12 | H | tBu | A-34 | H | A-34 | H |
| 44732 | 1 | H | m-D12 | H | m-D12 | H | tBu | A-35 | H | A-35 | H |
| 44733 | 1 | H | m-D12 | H | m-D12 | H | tBu | A-36 | H | A-36 | H |
| 44734 | 1 | H | m-D12 | H | m-D12 | H | tBu | A-37 | H | A-37 | H |
| 44735 | 1 | H | m-D12 | H | m-D12 | H | tBu | A-38 | H | A-38 | H |
| 44736 | 1 | H | m-D12 | H | m-D12 | H | tBu | A-39 | H | A-39 | H |
| 44737 | 1 | H | m-D12 | H | m-D12 | H | tBu | A-40 | H | A-40 | H |
| 44738 | 1 | H | m-D12 | H | m-D12 | H | tBu | A-41 | H | A-41 | H |
| 44739 | 1 | H | m-D12 | H | m-D12 | H | tBu | A-42 | H | A-42 | H |
| 44740 | 1 | H | m-D12 | H | m-D12 | H | tBu | A-43 | H | A-43 | H |
| 44741 | 1 | H | m-D12 | H | m-D12 | H | tBu | A-44 | H | A-44 | H |
| 44742 | 1 | H | m-D12 | H | m-D12 | H | tBu | A-45 | H | A-45 | H |
| 44743 | 1 | H | m-D12 | H | m-D12 | H | tBu | A-46 | H | A-46 | H |
| 44744 | 1 | H | m-D12 | H | m-D12 | H | tBu | A-47 | H | A-47 | H |
| 44745 | 1 | H | m-D13 | H | m-D13 | H | tBu | A-1 | H | A-1 | H |
| 44746 | 1 | H | m-D13 | H | m-D13 | H | tBu | A-2 | H | A-2 | H |
| 44747 | 1 | H | m-D13 | H | m-D13 | H | tBu | A-3 | H | A-3 | H |
| 44748 | 1 | H | m-D13 | H | m-D13 | H | tBu | A-4 | H | A-4 | H |
| 44749 | 1 | H | m-D13 | H | m-D13 | H | tBu | A-5 | H | A-5 | H |
| 44750 | 1 | H | m-D13 | H | m-D13 | H | tBu | A-6 | H | A-6 | H |
| 44751 | 1 | H | m-D13 | H | m-D13 | H | tBu | A-7 | H | A-7 | H |
| 44752 | 1 | H | m-D13 | H | m-D13 | H | tBu | A-8 | H | A-8 | H |
| 44753 | 1 | H | m-D13 | H | m-D13 | H | tBu | A-9 | H | A-9 | H |
| 44754 | 1 | H | m-D13 | H | m-D13 | H | tBu | A-10 | H | A-10 | H |
| 44755 | 1 | H | m-D13 | H | m-D13 | H | tBu | A-11 | H | A-11 | H |
| 44756 | 1 | H | m-D13 | H | m-D13 | H | tBu | A-12 | H | A-12 | H |
| 44757 | 1 | H | m-D13 | H | m-D13 | H | tBu | A-13 | H | A-13 | H |
| 44758 | 1 | H | m-D13 | H | m-D13 | H | tBu | A-14 | H | A-14 | H |
| 44759 | 1 | H | m-D13 | H | m-D13 | H | tBu | A-15 | H | A-15 | H |
| 44760 | 1 | H | m-D13 | H | m-D13 | H | tBu | A-16 | H | A-16 | H |
| 44761 | 1 | H | m-D13 | H | m-D13 | H | tBu | A-17 | H | A-17 | H |
| 44762 | 1 | H | m-D13 | H | m-D13 | H | tBu | A-18 | H | A-18 | H |
| 44763 | 1 | H | m-D13 | H | m-D13 | H | tBu | A-19 | H | A-19 | H |
| 44764 | 1 | H | m-D13 | H | m-D13 | H | tBu | A-20 | H | A-20 | H |
| 44765 | 1 | H | m-D13 | H | m-D13 | H | tBu | A-21 | H | A-21 | H |
| 44766 | 1 | H | m-D13 | H | m-D13 | H | tBu | A-22 | H | A-22 | H |
| 44767 | 1 | H | m-D13 | H | m-D13 | H | tBu | A-23 | H | A-23 | H |
| 44768 | 1 | H | m-D13 | H | m-D13 | H | tBu | A-24 | H | A-24 | H |
| 44769 | 1 | H | m-D13 | H | m-D13 | H | tBu | A-25 | H | A-25 | H |
| 44770 | 1 | H | m-D13 | H | m-D13 | H | tBu | A-26 | H | A-26 | H |
| 44771 | 1 | H | m-D13 | H | m-D13 | H | tBu | A-27 | H | A-27 | H |
| 44772 | 1 | H | m-D13 | H | m-D13 | H | tBu | A-28 | H | A-28 | H |
| 44773 | 1 | H | m-D13 | H | m-D13 | H | tBu | A-29 | H | A-29 | H |
| 44774 | 1 | H | m-D13 | H | m-D13 | H | tBu | A-30 | H | A-30 | H |
| 44775 | 1 | H | m-D13 | H | m-D13 | H | tBu | A-31 | H | A-31 | H |
| 44776 | 1 | H | m-D13 | H | m-D13 | H | tBu | A-32 | H | A-32 | H |
| 44777 | 1 | H | m-D13 | H | m-D13 | H | tBu | A-33 | H | A-33 | H |
| 44778 | 1 | H | m-D13 | H | m-D13 | H | tBu | A-34 | H | A-34 | H |
| 44779 | 1 | H | m-D13 | H | m-D13 | H | tBu | A-35 | H | A-35 | H |
| 44780 | 1 | H | m-D13 | H | m-D13 | H | tBu | A-36 | H | A-36 | H |
| 44781 | 1 | H | m-D13 | H | m-D13 | H | tBu | A-37 | H | A-37 | H |
| 44782 | 1 | H | m-D13 | H | m-D13 | H | tBu | A-38 | H | A-38 | H |
| 44783 | 1 | H | m-D13 | H | m-D13 | H | tBu | A-39 | H | A-39 | H |
| 44784 | 1 | H | m-D13 | H | m-D13 | H | tBu | A-40 | H | A-40 | H |
| 44785 | 1 | H | m-D13 | H | m-D13 | H | tBu | A-41 | H | A-41 | H |
| 44786 | 1 | H | m-D13 | H | m-D13 | H | tBu | A-42 | H | A-42 | H |
| 44787 | 1 | H | m-D13 | H | m-D13 | H | tBu | A-43 | H | A-43 | H |
| 44788 | 1 | H | m-D13 | H | m-D13 | H | tBu | A-44 | H | A-44 | H |
| 44789 | 1 | H | m-D13 | H | m-D13 | H | tBu | A-45 | H | A-45 | H |
| 44790 | 1 | H | m-D13 | H | m-D13 | H | tBu | A-46 | H | A-46 | H |
| 44791 | 1 | H | m-D13 | H | m-D13 | H | tBu | A-47 | H | A-47 | H |
| 44792 | 1 | H | m-D14 | H | m-D14 | H | tBu | A-1 | H | A-1 | H |
| 44793 | 1 | H | m-D14 | H | m-D14 | H | tBu | A-2 | H | A-2 | H |
| 44794 | 1 | H | m-D14 | H | m-D14 | H | tBu | A-3 | H | A-3 | H |
| 44795 | 1 | H | m-D14 | H | m-D14 | H | tBu | A-4 | H | A-4 | H |
| 44796 | 1 | H | m-D14 | H | m-D14 | H | tBu | A-5 | H | A-5 | H |
| 44797 | 1 | H | m-D14 | H | m-D14 | H | tBu | A-6 | H | A-6 | H |
| 44798 | 1 | H | m-D14 | H | m-D14 | H | tBu | A-7 | H | A-7 | H |
| 44799 | 1 | H | m-D14 | H | m-D14 | H | tBu | A-8 | H | A-8 | H |
| 44800 | 1 | H | m-D14 | H | m-D14 | H | tBu | A-9 | H | A-9 | H |
| 44801 | 1 | H | m-D14 | H | m-D14 | H | tBu | A-10 | H | A-10 | H |
| 44802 | 1 | H | m-D14 | H | m-D14 | H | tBu | A-11 | H | A-11 | H |
| 44803 | 1 | H | m-D14 | H | m-D14 | H | tBu | A-12 | H | A-12 | H |
| 44804 | 1 | H | m-D14 | H | m-D14 | H | tBu | A-13 | H | A-13 | H |
| 44805 | 1 | H | m-D14 | H | m-D14 | H | tBu | A-14 | H | A-14 | H |
| 44806 | 1 | H | m-D14 | H | m-D14 | H | tBu | A-15 | H | A-15 | H |
| 44807 | 1 | H | m-D14 | H | m-D14 | H | tBu | A-16 | H | A-16 | H |
| 44808 | 1 | H | m-D14 | H | m-D14 | H | tBu | A-17 | H | A-17 | H |
| 44809 | 1 | H | m-D14 | H | m-D14 | H | tBu | A-18 | H | A-18 | H |
| 44810 | 1 | H | m-D14 | H | m-D14 | H | tBu | A-19 | H | A-19 | H |
| 44811 | 1 | H | m-D14 | H | m-D14 | H | tBu | A-20 | H | A-20 | H |
| 44812 | 1 | H | m-D14 | H | m-D14 | H | tBu | A-21 | H | A-21 | H |
| 44813 | 1 | H | m-D14 | H | m-D14 | H | tBu | A-22 | H | A-22 | H |
| 44814 | 1 | H | m-D14 | H | m-D14 | H | tBu | A-23 | H | A-23 | H |
| 44815 | 1 | H | m-D14 | H | m-D14 | H | tBu | A-24 | H | A-24 | H |
| 44816 | 1 | H | m-D14 | H | m-D14 | H | tBu | A-25 | H | A-25 | H |
| 44817 | 1 | H | m-D14 | H | m-D14 | H | tBu | A-26 | H | A-26 | H |
| 44818 | 1 | H | m-D14 | H | m-D14 | H | tBu | A-27 | H | A-27 | H |
| 44819 | 1 | H | m-D14 | H | m-D14 | H | tBu | A-28 | H | A-28 | H |
| 44820 | 1 | H | m-D14 | H | m-D14 | H | tBu | A-29 | H | A-29 | H |
| 44821 | 1 | H | m-D14 | H | m-D14 | H | tBu | A-30 | H | A-30 | H |
| 44822 | 1 | H | m-D14 | H | m-D14 | H | tBu | A-31 | H | A-31 | H |
| 44823 | 1 | H | m-D14 | H | m-D14 | H | tBu | A-32 | H | A-32 | H |
| 44824 | 1 | H | m-D14 | H | m-D14 | H | tBu | A-33 | H | A-33 | H |
| 44825 | 1 | H | m-D14 | H | m-D14 | H | tBu | A-34 | H | A-34 | H |
| 44826 | 1 | H | m-D14 | H | m-D14 | H | tBu | A-35 | H | A-35 | H |
| 44827 | 1 | H | m-D14 | H | m-D14 | H | tBu | A-36 | H | A-36 | H |
| 44828 | 1 | H | m-D14 | H | m-D14 | H | tBu | A-37 | H | A-37 | H |
| 44829 | 1 | H | m-D14 | H | m-D14 | H | tBu | A-38 | H | A-38 | H |
| 44830 | 1 | H | m-D14 | H | m-D14 | H | tBu | A-39 | H | A-39 | H |
| 44831 | 1 | H | m-D14 | H | m-D14 | H | tBu | A-40 | H | A-40 | H |
| 44832 | 1 | H | m-D14 | H | m-D14 | H | tBu | A-41 | H | A-41 | H |
| 44833 | 1 | H | m-D14 | H | m-D14 | H | tBu | A-42 | H | A-42 | H |
| 44834 | 1 | H | m-D14 | H | m-D14 | H | tBu | A-43 | H | A-43 | H |
| 44835 | 1 | H | m-D14 | H | m-D14 | H | tBu | A-44 | H | A-44 | H |
| 44836 | 1 | H | m-D14 | H | m-D14 | H | tBu | A-45 | H | A-45 | H |
| 44837 | 1 | H | m-D14 | H | m-D14 | H | tBu | A-46 | H | A-46 | H |
| 44838 | 1 | H | m-D14 | H | m-D14 | H | tBu | A-47 | H | A-47 | H |
| 44839 | 1 | H | m-D1 | H | A-1 | H | tBu | A-1 | H | m-D1 | H |
| 44840 | 1 | H | m-D1 | H | A-2 | H | tBu | A-2 | H | m-D1 | H |
| 44841 | 1 | H | m-D1 | H | A-3 | H | tBu | A-3 | H | m-D1 | H |
| 44842 | 1 | H | m-D1 | H | A-4 | H | tBu | A-4 | H | m-D1 | H |
| 44843 | 1 | H | m-D1 | H | A-5 | H | tBu | A-5 | H | m-D1 | H |
| 44844 | 1 | H | m-D1 | H | A-6 | H | tBu | A-6 | H | m-D1 | H |
| 44845 | 1 | H | m-D1 | H | A-7 | H | tBu | A-7 | H | m-D1 | H |
| 44846 | 1 | H | m-D1 | H | A-8 | H | tBu | A-8 | H | m-D1 | H |
| 44847 | 1 | H | m-D1 | H | A-9 | H | tBu | A-9 | H | m-D1 | H |
| 44848 | 1 | H | m-D1 | H | A-10 | H | tBu | A-10 | H | m-D1 | H |
| 44849 | 1 | H | m-D1 | H | A-11 | H | tBu | A-11 | H | m-D1 | H |
| 44850 | 1 | H | m-D1 | H | A-12 | H | tBu | A-12 | H | m-D1 | H |
| 44851 | 1 | H | m-D1 | H | A-13 | H | tBu | A-13 | H | m-D1 | H |
| 44852 | 1 | H | m-D1 | H | A-14 | H | tBu | A-14 | H | m-D1 | H |
| 44853 | 1 | H | m-D1 | H | A-15 | H | tBu | A-15 | H | m-D1 | H |
| 44854 | 1 | H | m-D1 | H | A-16 | H | tBu | A-16 | H | m-D1 | H |
| 44855 | 1 | H | m-D1 | H | A-17 | H | tBu | A-17 | H | m-D1 | H |
| 44856 | 1 | H | m-D1 | H | A-18 | H | tBu | A-18 | H | m-D1 | H |
| 44857 | 1 | H | m-D1 | H | A-19 | H | tBu | A-19 | H | m-D1 | H |
| 44858 | 1 | H | m-D1 | H | A-20 | H | tBu | A-20 | H | m-D1 | H |
| 44859 | 1 | H | m-D1 | H | A-21 | H | tBu | A-21 | H | m-D1 | H |
| 44860 | 1 | H | m-D1 | H | A-22 | H | tBu | A-22 | H | m-D1 | H |
| 44861 | 1 | H | m-D1 | H | A-23 | H | tBu | A-23 | H | m-D1 | H |
| 44862 | 1 | H | m-D1 | H | A-24 | H | tBu | A-24 | H | m-D1 | H |
| 44863 | 1 | H | m-D1 | H | A-25 | H | tBu | A-25 | H | m-D1 | H |
| 44864 | 1 | H | m-D1 | H | A-26 | H | tBu | A-26 | H | m-D1 | H |
| 44865 | 1 | H | m-D1 | H | A-27 | H | tBu | A-27 | H | m-D1 | H |
| 44866 | 1 | H | m-D1 | H | A-28 | H | tBu | A-28 | H | m-D1 | H |
| 44867 | 1 | H | m-D1 | H | A-29 | H | tBu | A-29 | H | m-D1 | H |
| 44868 | 1 | H | m-D1 | H | A-30 | H | tBu | A-30 | H | m-D1 | H |
| 44869 | 1 | H | m-D1 | H | A-31 | H | tBu | A-31 | H | m-D1 | H |
| 44870 | 1 | H | m-D1 | H | A-32 | H | tBu | A-32 | H | m-D1 | H |
| 44871 | 1 | H | m-D1 | H | A-33 | H | tBu | A-33 | H | m-D1 | H |
| 44872 | 1 | H | m-D1 | H | A-34 | H | tBu | A-34 | H | m-D1 | H |
| 44873 | 1 | H | m-D1 | H | A-35 | H | tBu | A-35 | H | m-D1 | H |
| 44874 | 1 | H | m-D1 | H | A-36 | H | tBu | A-36 | H | m-D1 | H |
| 44875 | 1 | H | m-D1 | H | A-37 | H | tBu | A-37 | H | m-D1 | H |
| 44876 | 1 | H | m-D1 | H | A-38 | H | tBu | A-38 | H | m-D1 | H |
| 44877 | 1 | H | m-D1 | H | A-39 | H | tBu | A-39 | H | m-D1 | H |
| 44878 | 1 | H | m-D1 | H | A-40 | H | tBu | A-40 | H | m-D1 | H |
| 44879 | 1 | H | m-D1 | H | A-41 | H | tBu | A-41 | H | m-D1 | H |
| 44880 | 1 | H | m-D1 | H | A-42 | H | tBu | A-42 | H | m-D1 | H |
| 44881 | 1 | H | m-D1 | H | A-43 | H | tBu | A-43 | H | m-D1 | H |
| 44882 | 1 | H | m-D1 | H | A-44 | H | tBu | A-44 | H | m-D1 | H |
| 44883 | 1 | H | m-D1 | H | A-45 | H | tBu | A-45 | H | m-D1 | H |
| 44884 | 1 | H | m-D1 | H | A-46 | H | tBu | A-46 | H | m-D1 | H |
| 44885 | 1 | H | m-D1 | H | A-47 | H | tBu | A-47 | H | m-D1 | H |
| 44886 | 1 | H | m-D2 | H | A-1 | H | tBu | A-1 | H | m-D2 | H |
| 44887 | 1 | H | m-D2 | H | A-2 | H | tBu | A-2 | H | m-D2 | H |
| 44888 | 1 | H | m-D2 | H | A-3 | H | tBu | A-3 | H | m-D2 | H |
| 44889 | 1 | H | m-D2 | H | A-4 | H | tBu | A-4 | H | m-D2 | H |
| 44890 | 1 | H | m-D2 | H | A-5 | H | tBu | A-5 | H | m-D2 | H |
| 44891 | 1 | H | m-D2 | H | A-6 | H | tBu | A-6 | H | m-D2 | H |
| 44892 | 1 | H | m-D2 | H | A-7 | H | tBu | A-7 | H | m-D2 | H |
| 44893 | 1 | H | m-D2 | H | A-8 | H | tBu | A-8 | H | m-D2 | H |
| 44894 | 1 | H | m-D2 | H | A-9 | H | tBu | A-9 | H | m-D2 | H |
| 44895 | 1 | H | m-D2 | H | A-10 | H | tBu | A-10 | H | m-D2 | H |
| 44896 | 1 | H | m-D2 | H | A-11 | H | tBu | A-11 | H | m-D2 | H |
| 44897 | 1 | H | m-D2 | H | A-12 | H | tBu | A-12 | H | m-D2 | H |
| 44898 | 1 | H | m-D2 | H | A-13 | H | tBu | A-13 | H | m-D2 | H |
| 44899 | 1 | H | m-D2 | H | A-14 | H | tBu | A-14 | H | m-D2 | H |
| 44900 | 1 | H | m-D2 | H | A-15 | H | tBu | A-15 | H | m-D2 | H |
| 44901 | 1 | H | m-D2 | H | A-16 | H | tBu | A-16 | H | m-D2 | H |
| 44902 | 1 | H | m-D2 | H | A-17 | H | tBu | A-17 | H | m-D2 | H |
| 44903 | 1 | H | m-D2 | H | A-18 | H | tBu | A-18 | H | m-D2 | H |
| 44904 | 1 | H | m-D2 | H | A-19 | H | tBu | A-19 | H | m-D2 | H |
| 44905 | 1 | H | m-D2 | H | A-20 | H | tBu | A-20 | H | m-D2 | H |
| 44906 | 1 | H | m-D2 | H | A-21 | H | tBu | A-21 | H | m-D2 | H |
| 44907 | 1 | H | m-D2 | H | A-22 | H | tBu | A-22 | H | m-D2 | H |
| 44908 | 1 | H | m-D2 | H | A-23 | H | tBu | A-23 | H | m-D2 | H |
| 44909 | 1 | H | m-D2 | H | A-24 | H | tBu | A-24 | H | m-D2 | H |
| 44910 | 1 | H | m-D2 | H | A-25 | H | tBu | A-25 | H | m-D2 | H |
| 44911 | 1 | H | m-D2 | H | A-26 | H | tBu | A-26 | H | m-D2 | H |
| 44912 | 1 | H | m-D2 | H | A-27 | H | tBu | A-27 | H | m-D2 | H |
| 44913 | 1 | H | m-D2 | H | A-28 | H | tBu | A-28 | H | m-D2 | H |
| 44914 | 1 | H | m-D2 | H | A-29 | H | tBu | A-29 | H | m-D2 | H |
| 44915 | 1 | H | m-D2 | H | A-30 | H | tBu | A-30 | H | m-D2 | H |
| 44916 | 1 | H | m-D2 | H | A-31 | H | tBu | A-31 | H | m-D2 | H |
| 44917 | 1 | H | m-D2 | H | A-32 | H | tBu | A-32 | H | m-D2 | H |
| 44918 | 1 | H | m-D2 | H | A-33 | H | tBu | A-33 | H | m-D2 | H |
| 44919 | 1 | H | m-D2 | H | A-34 | H | tBu | A-34 | H | m-D2 | H |
| 44920 | 1 | H | m-D2 | H | A-35 | H | tBu | A-35 | H | m-D2 | H |
| 44921 | 1 | H | m-D2 | H | A-36 | H | tBu | A-36 | H | m-D2 | H |
| 44922 | 1 | H | m-D2 | H | A-37 | H | tBu | A-37 | H | m-D2 | H |
| 44923 | 1 | H | m-D2 | H | A-38 | H | tBu | A-38 | H | m-D2 | H |
| 44924 | 1 | H | m-D2 | H | A-39 | H | tBu | A-39 | H | m-D2 | H |
| 44925 | 1 | H | m-D2 | H | A-40 | H | tBu | A-40 | H | m-D2 | H |
| 44926 | 1 | H | m-D2 | H | A-41 | H | tBu | A-41 | H | m-D2 | H |
| 44927 | 1 | H | m-D2 | H | A-42 | H | tBu | A-42 | H | m-D2 | H |
| 44928 | 1 | H | m-D2 | H | A-43 | H | tBu | A-43 | H | m-D2 | H |
| 44929 | 1 | H | m-D2 | H | A-44 | H | tBu | A-44 | H | m-D2 | H |
| 44930 | 1 | H | m-D2 | H | A-45 | H | tBu | A-45 | H | m-D2 | H |
| 44931 | 1 | H | m-D2 | H | A-46 | H | tBu | A-46 | H | m-D2 | H |
| 44932 | 1 | H | m-D2 | H | A-47 | H | tBu | A-47 | H | m-D2 | H |
| 44933 | 1 | H | m-D3 | H | A-1 | H | tBu | A-1 | H | m-D3 | H |
| 44934 | 1 | H | m-D3 | H | A-2 | H | tBu | A-2 | H | m-D3 | H |
| 44935 | 1 | H | m-D3 | H | A-3 | H | tBu | A-3 | H | m-D3 | H |
| 44936 | 1 | H | m-D3 | H | A-4 | H | tBu | A-4 | H | m-D3 | H |
| 44937 | 1 | H | m-D3 | H | A-5 | H | tBu | A-5 | H | m-D3 | H |
| 44938 | 1 | H | m-D3 | H | A-6 | H | tBu | A-6 | H | m-D3 | H |
| 44939 | 1 | H | m-D3 | H | A-7 | H | tBu | A-7 | H | m-D3 | H |
| 44940 | 1 | H | m-D3 | H | A-8 | H | tBu | A-8 | H | m-D3 | H |
| 44941 | 1 | H | m-D3 | H | A-9 | H | tBu | A-9 | H | m-D3 | H |
| 44942 | 1 | H | m-D3 | H | A-10 | H | tBu | A-10 | H | m-D3 | H |
| 44943 | 1 | H | m-D3 | H | A-11 | H | tBu | A-11 | H | m-D3 | H |
| 44944 | 1 | H | m-D3 | H | A-12 | H | tBu | A-12 | H | m-D3 | H |
| 44945 | 1 | H | m-D3 | H | A-13 | H | tBu | A-13 | H | m-D3 | H |
| 44946 | 1 | H | m-D3 | H | A-14 | H | tBu | A-14 | H | m-D3 | H |
| 44947 | 1 | H | m-D3 | H | A-15 | H | tBu | A-15 | H | m-D3 | H |
| 44948 | 1 | H | m-D3 | H | A-16 | H | tBu | A-16 | H | m-D3 | H |
| 44949 | 1 | H | m-D3 | H | A-17 | H | tBu | A-17 | H | m-D3 | H |
| 44950 | 1 | H | m-D3 | H | A-18 | H | tBu | A-18 | H | m-D3 | H |
| 44951 | 1 | H | m-D3 | H | A-19 | H | tBu | A-19 | H | m-D3 | H |
| 44952 | 1 | H | m-D3 | H | A-20 | H | tBu | A-20 | H | m-D3 | H |
| 44953 | 1 | H | m-D3 | H | A-21 | H | tBu | A-21 | H | m-D3 | H |
| 44954 | 1 | H | m-D3 | H | A-22 | H | tBu | A-22 | H | m-D3 | H |
| 44955 | 1 | H | m-D3 | H | A-23 | H | tBu | A-23 | H | m-D3 | H |
| 44956 | 1 | H | m-D3 | H | A-24 | H | tBu | A-24 | H | m-D3 | H |
| 44957 | 1 | H | m-D3 | H | A-25 | H | tBu | A-25 | H | m-D3 | H |
| 44958 | 1 | H | m-D3 | H | A-26 | H | tBu | A-26 | H | m-D3 | H |
| 44959 | 1 | H | m-D3 | H | A-27 | H | tBu | A-27 | H | m-D3 | H |
| 44960 | 1 | H | m-D3 | H | A-28 | H | tBu | A-28 | H | m-D3 | H |
| 44961 | 1 | H | m-D3 | H | A-29 | H | tBu | A-29 | H | m-D3 | H |
| 44962 | 1 | H | m-D3 | H | A-30 | H | tBu | A-30 | H | m-D3 | H |
| 44963 | 1 | H | m-D3 | H | A-31 | H | tBu | A-31 | H | m-D3 | H |
| 44964 | 1 | H | m-D3 | H | A-32 | H | tBu | A-32 | H | m-D3 | H |
| 44965 | 1 | H | m-D3 | H | A-33 | H | tBu | A-33 | H | m-D3 | H |
| 44966 | 1 | H | m-D3 | H | A-34 | H | tBu | A-34 | H | m-D3 | H |
| 44967 | 1 | H | m-D3 | H | A-35 | H | tBu | A-35 | H | m-D3 | H |
| 44968 | 1 | H | m-D3 | H | A-36 | H | tBu | A-36 | H | m-D3 | H |
| 44969 | 1 | H | m-D3 | H | A-37 | H | tBu | A-37 | H | m-D3 | H |
| 44970 | 1 | H | m-D3 | H | A-38 | H | tBu | A-38 | H | m-D3 | H |
| 44971 | 1 | H | m-D3 | H | A-39 | H | tBu | A-39 | H | m-D3 | H |
| 44972 | 1 | H | m-D3 | H | A-40 | H | tBu | A-40 | H | m-D3 | H |
| 44973 | 1 | H | m-D3 | H | A-41 | H | tBu | A-41 | H | m-D3 | H |
| 44974 | 1 | H | m-D3 | H | A-42 | H | tBu | A-42 | H | m-D3 | H |
| 44975 | 1 | H | m-D3 | H | A-43 | H | tBu | A-43 | H | m-D3 | H |
| 44976 | 1 | H | m-D3 | H | A-44 | H | tBu | A-44 | H | m-D3 | H |
| 44977 | 1 | H | m-D3 | H | A-45 | H | tBu | A-45 | H | m-D3 | H |
| 44978 | 1 | H | m-D3 | H | A-46 | H | tBu | A-46 | H | m-D3 | H |
| 44979 | 1 | H | m-D3 | H | A-47 | H | tBu | A-47 | H | m-D3 | H |
| 44980 | 1 | H | m-D4 | H | A-1 | H | tBu | A-1 | H | m-D4 | H |
| 44981 | 1 | H | m-D4 | H | A-2 | H | tBu | A-2 | H | m-D4 | H |
| 44982 | 1 | H | m-D4 | H | A-3 | H | tBu | A-3 | H | m-D4 | H |
| 44983 | 1 | H | m-D4 | H | A-4 | H | tBu | A-4 | H | m-D4 | H |
| 44984 | 1 | H | m-D4 | H | A-5 | H | tBu | A-5 | H | m-D4 | H |
| 44985 | 1 | H | m-D4 | H | A-6 | H | tBu | A-6 | H | m-D4 | H |
| 44986 | 1 | H | m-D4 | H | A-7 | H | tBu | A-7 | H | m-D4 | H |
| 44987 | 1 | H | m-D4 | H | A-8 | H | tBu | A-8 | H | m-D4 | H |
| 44988 | 1 | H | m-D4 | H | A-9 | H | tBu | A-9 | H | m-D4 | H |
| 44989 | 1 | H | m-D4 | H | A-10 | H | tBu | A-10 | H | m-D4 | H |
| 44990 | 1 | H | m-D4 | H | A-11 | H | tBu | A-11 | H | m-D4 | H |
| 44991 | 1 | H | m-D4 | H | A-12 | H | tBu | A-12 | H | m-D4 | H |
| 44992 | 1 | H | m-D4 | H | A-13 | H | tBu | A-13 | H | m-D4 | H |
| 44993 | 1 | H | m-D4 | H | A-14 | H | tBu | A-14 | H | m-D4 | H |
| 44994 | 1 | H | m-D4 | H | A-15 | H | tBu | A-15 | H | m-D4 | H |
| 44995 | 1 | H | m-D4 | H | A-16 | H | tBu | A-16 | H | m-D4 | H |
| 44996 | 1 | H | m-D4 | H | A-17 | H | tBu | A-17 | H | m-D4 | H |
| 44997 | 1 | H | m-D4 | H | A-18 | H | tBu | A-18 | H | m-D4 | H |
| 44998 | 1 | H | m-D4 | H | A-19 | H | tBu | A-19 | H | m-D4 | H |
| 44999 | 1 | H | m-D4 | H | A-20 | H | tBu | A-20 | H | m-D4 | H |
| 45000 | 1 | H | m-D4 | H | A-21 | H | tBu | A-21 | H | m-D4 | H |
| 45001 | 1 | H | m-D4 | H | A-22 | H | tBu | A-22 | H | m-D4 | H |
| 45002 | 1 | H | m-D4 | H | A-23 | H | tBu | A-23 | H | m-D4 | H |
| 45003 | 1 | H | m-D4 | H | A-24 | H | tBu | A-24 | H | m-D4 | H |
| 45004 | 1 | H | m-D4 | H | A-25 | H | tBu | A-25 | H | m-D4 | H |
| 45005 | 1 | H | m-D4 | H | A-26 | H | tBu | A-26 | H | m-D4 | H |
| 45006 | 1 | H | m-D4 | H | A-27 | H | tBu | A-27 | H | m-D4 | H |
| 45007 | 1 | H | m-D4 | H | A-28 | H | tBu | A-28 | H | m-D4 | H |
| 45008 | 1 | H | m-D4 | H | A-29 | H | tBu | A-29 | H | m-D4 | H |
| 45009 | 1 | H | m-D4 | H | A-30 | H | tBu | A-30 | H | m-D4 | H |
| 45010 | 1 | H | m-D4 | H | A-31 | H | tBu | A-31 | H | m-D4 | H |
| 45011 | 1 | H | m-D4 | H | A-32 | H | tBu | A-32 | H | m-D4 | H |
| 45012 | 1 | H | m-D4 | H | A-33 | H | tBu | A-33 | H | m-D4 | H |
| 45013 | 1 | H | m-D4 | H | A-34 | H | tBu | A-34 | H | m-D4 | H |
| 45014 | 1 | H | m-D4 | H | A-35 | H | tBu | A-35 | H | m-D4 | H |
| 45015 | 1 | H | m-D4 | H | A-36 | H | tBu | A-36 | H | m-D4 | H |
| 45016 | 1 | H | m-D4 | H | A-37 | H | tBu | A-37 | H | m-D4 | H |
| 45017 | 1 | H | m-D4 | H | A-38 | H | tBu | A-38 | H | m-D4 | H |
| 45018 | 1 | H | m-D4 | H | A-39 | H | tBu | A-39 | H | m-D4 | H |
| 45019 | 1 | H | m-D4 | H | A-40 | H | tBu | A-40 | H | m-D4 | H |
| 45020 | 1 | H | m-D4 | H | A-41 | H | tBu | A-41 | H | m-D4 | H |
| 45021 | 1 | H | m-D4 | H | A-42 | H | tBu | A-42 | H | m-D4 | H |
| 45022 | 1 | H | m-D4 | H | A-43 | H | tBu | A-43 | H | m-D4 | H |
| 45023 | 1 | H | m-D4 | H | A-44 | H | tBu | A-44 | H | m-D4 | H |
| 45024 | 1 | H | m-D4 | H | A-45 | H | tBu | A-45 | H | m-D4 | H |
| 45025 | 1 | H | m-D4 | H | A-46 | H | tBu | A-46 | H | m-D4 | H |
| 45026 | 1 | H | m-D4 | H | A-47 | H | tBu | A-47 | H | m-D4 | H |
| 45027 | 1 | H | m-D5 | H | A-1 | H | tBu | A-1 | H | m-D5 | H |
| 45028 | 1 | H | m-D5 | H | A-2 | H | tBu | A-2 | H | m-D5 | H |
| 45029 | 1 | H | m-D5 | H | A-3 | H | tBu | A-3 | H | m-D5 | H |
| 45030 | 1 | H | m-D5 | H | A-4 | H | tBu | A-4 | H | m-D5 | H |
| 45031 | 1 | H | m-D5 | H | A-5 | H | tBu | A-5 | H | m-D5 | H |
| 45032 | 1 | H | m-D5 | H | A-6 | H | tBu | A-6 | H | m-D5 | H |
| 45033 | 1 | H | m-D5 | H | A-7 | H | tBu | A-7 | H | m-D5 | H |
| 45034 | 1 | H | m-D5 | H | A-8 | H | tBu | A-8 | H | m-D5 | H |
| 45035 | 1 | H | m-D5 | H | A-9 | H | tBu | A-9 | H | m-D5 | H |
| 45036 | 1 | H | m-D5 | H | A-10 | H | tBu | A-10 | H | m-D5 | H |
| 45037 | 1 | H | m-D5 | H | A-11 | H | tBu | A-11 | H | m-D5 | H |
| 45038 | 1 | H | m-D5 | H | A-12 | H | tBu | A-12 | H | m-D5 | H |
| 45039 | 1 | H | m-D5 | H | A-13 | H | tBu | A-13 | H | m-D5 | H |
| 45040 | 1 | H | m-D5 | H | A-14 | H | tBu | A-14 | H | m-D5 | H |
| 45041 | 1 | H | m-D5 | H | A-15 | H | tBu | A-15 | H | m-D5 | H |
| 45042 | 1 | H | m-D5 | H | A-16 | H | tBu | A-16 | H | m-D5 | H |
| 45043 | 1 | H | m-D5 | H | A-17 | H | tBu | A-17 | H | m-D5 | H |
| 45044 | 1 | H | m-D5 | H | A-18 | H | tBu | A-18 | H | m-D5 | H |
| 45045 | 1 | H | m-D5 | H | A-19 | H | tBu | A-19 | H | m-D5 | H |
| 45046 | 1 | H | m-D5 | H | A-20 | H | tBu | A-20 | H | m-D5 | H |
| 45047 | 1 | H | m-D5 | H | A-21 | H | tBu | A-21 | H | m-D5 | H |
| 45048 | 1 | H | m-D5 | H | A-22 | H | tBu | A-22 | H | m-D5 | H |
| 45049 | 1 | H | m-D5 | H | A-23 | H | tBu | A-23 | H | m-D5 | H |
| 45050 | 1 | H | m-D5 | H | A-24 | H | tBu | A-24 | H | m-D5 | H |
| 45051 | 1 | H | m-D5 | H | A-25 | H | tBu | A-25 | H | m-D5 | H |
| 45052 | 1 | H | m-D5 | H | A-26 | H | tBu | A-26 | H | m-D5 | H |
| 45053 | 1 | H | m-D5 | H | A-27 | H | tBu | A-27 | H | m-D5 | H |
| 45054 | 1 | H | m-D5 | H | A-28 | H | tBu | A-28 | H | m-D5 | H |
| 45055 | 1 | H | m-D5 | H | A-29 | H | tBu | A-29 | H | m-D5 | H |
| 45056 | 1 | H | m-D5 | H | A-30 | H | tBu | A-30 | H | m-D5 | H |
| 45057 | 1 | H | m-D5 | H | A-31 | H | tBu | A-31 | H | m-D5 | H |
| 45058 | 1 | H | m-D5 | H | A-32 | H | tBu | A-32 | H | m-D5 | H |
| 45059 | 1 | H | m-D5 | H | A-33 | H | tBu | A-33 | H | m-D5 | H |
| 45060 | 1 | H | m-D5 | H | A-34 | H | tBu | A-34 | H | m-D5 | H |
| 45061 | 1 | H | m-D5 | H | A-35 | H | tBu | A-35 | H | m-D5 | H |
| 45062 | 1 | H | m-D5 | H | A-36 | H | tBu | A-36 | H | m-D5 | H |
| 45063 | 1 | H | m-D5 | H | A-37 | H | tBu | A-37 | H | m-D5 | H |
| 45064 | 1 | H | m-D5 | H | A-38 | H | tBu | A-38 | H | m-D5 | H |
| 45065 | 1 | H | m-D5 | H | A-39 | H | tBu | A-39 | H | m-D5 | H |
| 45066 | 1 | H | m-D5 | H | A-40 | H | tBu | A-40 | H | m-D5 | H |
| 45067 | 1 | H | m-D5 | H | A-41 | H | tBu | A-41 | H | m-D5 | H |
| 45068 | 1 | H | m-D5 | H | A-42 | H | tBu | A-42 | H | m-D5 | H |
| 45069 | 1 | H | m-D5 | H | A-43 | H | tBu | A-43 | H | m-D5 | H |
| 45070 | 1 | H | m-D5 | H | A-44 | H | tBu | A-44 | H | m-D5 | H |
| 45071 | 1 | H | m-D5 | H | A-45 | H | tBu | A-45 | H | m-D5 | H |
| 45072 | 1 | H | m-D5 | H | A-46 | H | tBu | A-46 | H | m-D5 | H |
| 45073 | 1 | H | m-D5 | H | A-47 | H | tBu | A-47 | H | m-D5 | H |
| 45074 | 1 | H | m-D6 | H | A-1 | H | tBu | A-1 | H | m-D6 | H |
| 45075 | 1 | H | m-D6 | H | A-2 | H | tBu | A-2 | H | m-D6 | H |
| 45076 | 1 | H | m-D6 | H | A-3 | H | tBu | A-3 | H | m-D6 | H |
| 45077 | 1 | H | m-D6 | H | A-4 | H | tBu | A-4 | H | m-D6 | H |
| 45078 | 1 | H | m-D6 | H | A-5 | H | tBu | A-5 | H | m-D6 | H |
| 45079 | 1 | H | m-D6 | H | A-6 | H | tBu | A-6 | H | m-D6 | H |
| 45080 | 1 | H | m-D6 | H | A-7 | H | tBu | A-7 | H | m-D6 | H |
| 45081 | 1 | H | m-D6 | H | A-8 | H | tBu | A-8 | H | m-D6 | H |
| 45082 | 1 | H | m-D6 | H | A-9 | H | tBu | A-9 | H | m-D6 | H |
| 45083 | 1 | H | m-D6 | H | A-10 | H | tBu | A-10 | H | m-D6 | H |
| 45084 | 1 | H | m-D6 | H | A-11 | H | tBu | A-11 | H | m-D6 | H |
| 45085 | 1 | H | m-D6 | H | A-12 | H | tBu | A-12 | H | m-D6 | H |
| 45086 | 1 | H | m-D6 | H | A-13 | H | tBu | A-13 | H | m-D6 | H |
| 45087 | 1 | H | m-D6 | H | A-14 | H | tBu | A-14 | H | m-D6 | H |
| 45088 | 1 | H | m-D6 | H | A-15 | H | tBu | A-15 | H | m-D6 | H |
| 45089 | 1 | H | m-D6 | H | A-16 | H | tBu | A-16 | H | m-D6 | H |
| 45090 | 1 | H | m-D6 | H | A-17 | H | tBu | A-17 | H | m-D6 | H |
| 45091 | 1 | H | m-D6 | H | A-18 | H | tBu | A-18 | H | m-D6 | H |
| 45092 | 1 | H | m-D6 | H | A-19 | H | tBu | A-19 | H | m-D6 | H |
| 45093 | 1 | H | m-D6 | H | A-20 | H | tBu | A-20 | H | m-D6 | H |
| 45094 | 1 | H | m-D6 | H | A-21 | H | tBu | A-21 | H | m-D6 | H |
| 45095 | 1 | H | m-D6 | H | A-22 | H | tBu | A-22 | H | m-D6 | H |
| 45096 | 1 | H | m-D6 | H | A-23 | H | tBu | A-23 | H | m-D6 | H |
| 45097 | 1 | H | m-D6 | H | A-24 | H | tBu | A-24 | H | m-D6 | H |
| 45098 | 1 | H | m-D6 | H | A-25 | H | tBu | A-25 | H | m-D6 | H |
| 45099 | 1 | H | m-D6 | H | A-26 | H | tBu | A-26 | H | m-D6 | H |
| 45100 | 1 | H | m-D6 | H | A-27 | H | tBu | A-27 | H | m-D6 | H |
| 45101 | 1 | H | m-D6 | H | A-28 | H | tBu | A-28 | H | m-D6 | H |
| 45102 | 1 | H | m-D6 | H | A-29 | H | tBu | A-29 | H | m-D6 | H |
| 45103 | 1 | H | m-D6 | H | A-30 | H | tBu | A-30 | H | m-D6 | H |
| 45104 | 1 | H | m-D6 | H | A-31 | H | tBu | A-31 | H | m-D6 | H |
| 45105 | 1 | H | m-D6 | H | A-32 | H | tBu | A-32 | H | m-D6 | H |
| 45106 | 1 | H | m-D6 | H | A-33 | H | tBu | A-33 | H | m-D6 | H |
| 45107 | 1 | H | m-D6 | H | A-34 | H | tBu | A-34 | H | m-D6 | H |
| 45108 | 1 | H | m-D6 | H | A-35 | H | tBu | A-35 | H | m-D6 | H |
| 45109 | 1 | H | m-D6 | H | A-36 | H | tBu | A-36 | H | m-D6 | H |
| 45110 | 1 | H | m-D6 | H | A-37 | H | tBu | A-37 | H | m-D6 | H |
| 45111 | 1 | H | m-D6 | H | A-38 | H | tBu | A-38 | H | m-D6 | H |
| 45112 | 1 | H | m-D6 | H | A-39 | H | tBu | A-39 | H | m-D6 | H |
| 45113 | 1 | H | m-D6 | H | A-40 | H | tBu | A-40 | H | m-D6 | H |
| 45114 | 1 | H | m-D6 | H | A-41 | H | tBu | A-41 | H | m-D6 | H |
| 45115 | 1 | H | m-D6 | H | A-42 | H | tBu | A-42 | H | m-D6 | H |
| 45116 | 1 | H | m-D6 | H | A-43 | H | tBu | A-43 | H | m-D6 | H |
| 45117 | 1 | H | m-D6 | H | A-44 | H | tBu | A-44 | H | m-D6 | H |
| 45118 | 1 | H | m-D6 | H | A-45 | H | tBu | A-45 | H | m-D6 | H |
| 45119 | 1 | H | m-D6 | H | A-46 | H | tBu | A-46 | H | m-D6 | H |
| 45120 | 1 | H | m-D6 | H | A-47 | H | tBu | A-47 | H | m-D6 | H |
| 45121 | 1 | H | m-D7 | H | A-1 | H | tBu | A-1 | H | m-D7 | H |
| 45122 | 1 | H | m-D7 | H | A-2 | H | tBu | A-2 | H | m-D7 | H |
| 45123 | 1 | H | m-D7 | H | A-3 | H | tBu | A-3 | H | m-D7 | H |
| 45124 | 1 | H | m-D7 | H | A-4 | H | tBu | A-4 | H | m-D7 | H |
| 45125 | 1 | H | m-D7 | H | A-5 | H | tBu | A-5 | H | m-D7 | H |
| 45126 | 1 | H | m-D7 | H | A-6 | H | tBu | A-6 | H | m-D7 | H |
| 45127 | 1 | H | m-D7 | H | A-7 | H | tBu | A-7 | H | m-D7 | H |
| 45128 | 1 | H | m-D7 | H | A-8 | H | tBu | A-8 | H | m-D7 | H |
| 45129 | 1 | H | m-D7 | H | A-9 | H | tBu | A-9 | H | m-D7 | H |
| 45130 | 1 | H | m-D7 | H | A-10 | H | tBu | A-10 | H | m-D7 | H |
| 45131 | 1 | H | m-D7 | H | A-11 | H | tBu | A-11 | H | m-D7 | H |
| 45132 | 1 | H | m-D7 | H | A-12 | H | tBu | A-12 | H | m-D7 | H |
| 45133 | 1 | H | m-D7 | H | A-13 | H | tBu | A-13 | H | m-D7 | H |
| 45134 | 1 | H | m-D7 | H | A-14 | H | tBu | A-14 | H | m-D7 | H |
| 45135 | 1 | H | m-D7 | H | A-15 | H | tBu | A-15 | H | m-D7 | H |
| 45136 | 1 | H | m-D7 | H | A-16 | H | tBu | A-16 | H | m-D7 | H |
| 45137 | 1 | H | m-D7 | H | A-17 | H | tBu | A-17 | H | m-D7 | H |
| 45138 | 1 | H | m-D7 | H | A-18 | H | tBu | A-18 | H | m-D7 | H |
| 45139 | 1 | H | m-D7 | H | A-19 | H | tBu | A-19 | H | m-D7 | H |
| 45140 | 1 | H | m-D7 | H | A-20 | H | tBu | A-20 | H | m-D7 | H |
| 45141 | 1 | H | m-D7 | H | A-21 | H | tBu | A-21 | H | m-D7 | H |
| 45142 | 1 | H | m-D7 | H | A-22 | H | tBu | A-22 | H | m-D7 | H |
| 45143 | 1 | H | m-D7 | H | A-23 | H | tBu | A-23 | H | m-D7 | H |
| 45144 | 1 | H | m-D7 | H | A-24 | H | tBu | A-24 | H | m-D7 | H |
| 45145 | 1 | H | m-D7 | H | A-25 | H | tBu | A-25 | H | m-D7 | H |
| 45146 | 1 | H | m-D7 | H | A-26 | H | tBu | A-26 | H | m-D7 | H |
| 45147 | 1 | H | m-D7 | H | A-27 | H | tBu | A-27 | H | m-D7 | H |
| 45148 | 1 | H | m-D7 | H | A-28 | H | tBu | A-28 | H | m-D7 | H |
| 45149 | 1 | H | m-D7 | H | A-29 | H | tBu | A-29 | H | m-D7 | H |
| 45150 | 1 | H | m-D7 | H | A-30 | H | tBu | A-30 | H | m-D7 | H |
| 45151 | 1 | H | m-D7 | H | A-31 | H | tBu | A-31 | H | m-D7 | H |
| 45152 | 1 | H | m-D7 | H | A-32 | H | tBu | A-32 | H | m-D7 | H |
| 45153 | 1 | H | m-D7 | H | A-33 | H | tBu | A-33 | H | m-D7 | H |
| 45154 | 1 | H | m-D7 | H | A-34 | H | tBu | A-34 | H | m-D7 | H |
| 45155 | 1 | H | m-D7 | H | A-35 | H | tBu | A-35 | H | m-D7 | H |
| 45156 | 1 | H | m-D7 | H | A-36 | H | tBu | A-36 | H | m-D7 | H |
| 45157 | 1 | H | m-D7 | H | A-37 | H | tBu | A-37 | H | m-D7 | H |
| 45158 | 1 | H | m-D7 | H | A-38 | H | tBu | A-38 | H | m-D7 | H |
| 45159 | 1 | H | m-D7 | H | A-39 | H | tBu | A-39 | H | m-D7 | H |
| 45160 | 1 | H | m-D7 | H | A-40 | H | tBu | A-40 | H | m-D7 | H |
| 45161 | 1 | H | m-D7 | H | A-41 | H | tBu | A-41 | H | m-D7 | H |
| 45162 | 1 | H | m-D7 | H | A-42 | H | tBu | A-42 | H | m-D7 | H |
| 45163 | 1 | H | m-D7 | H | A-43 | H | tBu | A-43 | H | m-D7 | H |
| 45164 | 1 | H | m-D7 | H | A-44 | H | tBu | A-44 | H | m-D7 | H |
| 45165 | 1 | H | m-D7 | H | A-45 | H | tBu | A-45 | H | m-D7 | H |
| 45166 | 1 | H | m-D7 | H | A-46 | H | tBu | A-46 | H | m-D7 | H |
| 45167 | 1 | H | m-D7 | H | A-47 | H | tBu | A-47 | H | m-D7 | H |
| 45168 | 1 | H | m-D8 | H | A-1 | H | tBu | A-1 | H | m-D8 | H |
| 45169 | 1 | H | m-D8 | H | A-2 | H | tBu | A-2 | H | m-D8 | H |
| 45170 | 1 | H | m-D8 | H | A-3 | H | tBu | A-3 | H | m-D8 | H |
| 45171 | 1 | H | m-D8 | H | A-4 | H | tBu | A-4 | H | m-D8 | H |
| 45172 | 1 | H | m-D8 | H | A-5 | H | tBu | A-5 | H | m-D8 | H |
| 45173 | 1 | H | m-D8 | H | A-6 | H | tBu | A-6 | H | m-D8 | H |
| 45174 | 1 | H | m-D8 | H | A-7 | H | tBu | A-7 | H | m-D8 | H |
| 45175 | 1 | H | m-D8 | H | A-8 | H | tBu | A-8 | H | m-D8 | H |
| 45176 | 1 | H | m-D8 | H | A-9 | H | tBu | A-9 | H | m-D8 | H |
| 45177 | 1 | H | m-D8 | H | A-10 | H | tBu | A-10 | H | m-D8 | H |
| 45178 | 1 | H | m-D8 | H | A-11 | H | tBu | A-11 | H | m-D8 | H |
| 45179 | 1 | H | m-D8 | H | A-12 | H | tBu | A-12 | H | m-D8 | H |
| 45180 | 1 | H | m-D8 | H | A-13 | H | tBu | A-13 | H | m-D8 | H |
| 45181 | 1 | H | m-D8 | H | A-14 | H | tBu | A-14 | H | m-D8 | H |
| 45182 | 1 | H | m-D8 | H | A-15 | H | tBu | A-15 | H | m-D8 | H |
| 45183 | 1 | H | m-D8 | H | A-16 | H | tBu | A-16 | H | m-D8 | H |
| 45184 | 1 | H | m-D8 | H | A-17 | H | tBu | A-17 | H | m-D8 | H |
| 45185 | 1 | H | m-D8 | H | A-18 | H | tBu | A-18 | H | m-D8 | H |
| 45186 | 1 | H | m-D8 | H | A-19 | H | tBu | A-19 | H | m-D8 | H |
| 45187 | 1 | H | m-D8 | H | A-20 | H | tBu | A-20 | H | m-D8 | H |
| 45188 | 1 | H | m-D8 | H | A-21 | H | tBu | A-21 | H | m-D8 | H |
| 45189 | 1 | H | m-D8 | H | A-22 | H | tBu | A-22 | H | m-D8 | H |
| 45190 | 1 | H | m-D8 | H | A-23 | H | tBu | A-23 | H | m-D8 | H |
| 45191 | 1 | H | m-D8 | H | A-24 | H | tBu | A-24 | H | m-D8 | H |
| 45192 | 1 | H | m-D8 | H | A-25 | H | tBu | A-25 | H | m-D8 | H |
| 45193 | 1 | H | m-D8 | H | A-26 | H | tBu | A-26 | H | m-D8 | H |
| 45194 | 1 | H | m-D8 | H | A-27 | H | tBu | A-27 | H | m-D8 | H |
| 45195 | 1 | H | m-D8 | H | A-28 | H | tBu | A-28 | H | m-D8 | H |
| 45196 | 1 | H | m-D8 | H | A-29 | H | tBu | A-29 | H | m-D8 | H |
| 45197 | 1 | H | m-D8 | H | A-30 | H | tBu | A-30 | H | m-D8 | H |
| 45198 | 1 | H | m-D8 | H | A-31 | H | tBu | A-31 | H | m-D8 | H |
| 45199 | 1 | H | m-D8 | H | A-32 | H | tBu | A-32 | H | m-D8 | H |
| 45200 | 1 | H | m-D8 | H | A-33 | H | tBu | A-33 | H | m-D8 | H |
| 45201 | 1 | H | m-D8 | H | A-34 | H | tBu | A-34 | H | m-D8 | H |
| 45202 | 1 | H | m-D8 | H | A-35 | H | tBu | A-35 | H | m-D8 | H |
| 45203 | 1 | H | m-D8 | H | A-36 | H | tBu | A-36 | H | m-D8 | H |
| 45204 | 1 | H | m-D8 | H | A-37 | H | tBu | A-37 | H | m-D8 | H |
| 45205 | 1 | H | m-D8 | H | A-38 | H | tBu | A-38 | H | m-D8 | H |
| 45206 | 1 | H | m-D8 | H | A-39 | H | tBu | A-39 | H | m-D8 | H |
| 45207 | 1 | H | m-D8 | H | A-40 | H | tBu | A-40 | H | m-D8 | H |
| 45208 | 1 | H | m-D8 | H | A-41 | H | tBu | A-41 | H | m-D8 | H |
| 45209 | 1 | H | m-D8 | H | A-42 | H | tBu | A-42 | H | m-D8 | H |
| 45210 | 1 | H | m-D8 | H | A-43 | H | tBu | A-43 | H | m-D8 | H |
| 45211 | 1 | H | m-D8 | H | A-44 | H | tBu | A-44 | H | m-D8 | H |
| 45212 | 1 | H | m-D8 | H | A-45 | H | tBu | A-45 | H | m-D8 | H |
| 45213 | 1 | H | m-D8 | H | A-46 | H | tBu | A-46 | H | m-D8 | H |
| 45214 | 1 | H | m-D8 | H | A-47 | H | tBu | A-47 | H | m-D8 | H |
| 45215 | 1 | H | m-D9 | H | A-1 | H | tBu | A-1 | H | m-D9 | H |
| 45216 | 1 | H | m-D9 | H | A-2 | H | tBu | A-2 | H | m-D9 | H |
| 45217 | 1 | H | m-D9 | H | A-3 | H | tBu | A-3 | H | m-D9 | H |
| 45218 | 1 | H | m-D9 | H | A-4 | H | tBu | A-4 | H | m-D9 | H |
| 45219 | 1 | H | m-D9 | H | A-5 | H | tBu | A-5 | H | m-D9 | H |
| 45220 | 1 | H | m-D9 | H | A-6 | H | tBu | A-6 | H | m-D9 | H |
| 45221 | 1 | H | m-D9 | H | A-7 | H | tBu | A-7 | H | m-D9 | H |
| 45222 | 1 | H | m-D9 | H | A-8 | H | tBu | A-8 | H | m-D9 | H |
| 45223 | 1 | H | m-D9 | H | A-9 | H | tBu | A-9 | H | m-D9 | H |
| 45224 | 1 | H | m-D9 | H | A-10 | H | tBu | A-10 | H | m-D9 | H |
| 45225 | 1 | H | m-D9 | H | A-11 | H | tBu | A-11 | H | m-D9 | H |
| 45226 | 1 | H | m-D9 | H | A-12 | H | tBu | A-12 | H | m-D9 | H |
| 45227 | 1 | H | m-D9 | H | A-13 | H | tBu | A-13 | H | m-D9 | H |
| 45228 | 1 | H | m-D9 | H | A-14 | H | tBu | A-14 | H | m-D9 | H |
| 45229 | 1 | H | m-D9 | H | A-15 | H | tBu | A-15 | H | m-D9 | H |
| 45230 | 1 | H | m-D9 | H | A-16 | H | tBu | A-16 | H | m-D9 | H |
| 45231 | 1 | H | m-D9 | H | A-17 | H | tBu | A-17 | H | m-D9 | H |
| 45232 | 1 | H | m-D9 | H | A-18 | H | tBu | A-18 | H | m-D9 | H |
| 45233 | 1 | H | m-D9 | H | A-19 | H | tBu | A-19 | H | m-D9 | H |
| 45234 | 1 | H | m-D9 | H | A-20 | H | tBu | A-20 | H | m-D9 | H |
| 45235 | 1 | H | m-D9 | H | A-21 | H | tBu | A-21 | H | m-D9 | H |
| 45236 | 1 | H | m-D9 | H | A-22 | H | tBu | A-22 | H | m-D9 | H |
| 45237 | 1 | H | m-D9 | H | A-23 | H | tBu | A-23 | H | m-D9 | H |
| 45238 | 1 | H | m-D9 | H | A-24 | H | tBu | A-24 | H | m-D9 | H |
| 45239 | 1 | H | m-D9 | H | A-25 | H | tBu | A-25 | H | m-D9 | H |
| 45240 | 1 | H | m-D9 | H | A-26 | H | tBu | A-26 | H | m-D9 | H |
| 45241 | 1 | H | m-D9 | H | A-27 | H | tBu | A-27 | H | m-D9 | H |
| 45242 | 1 | H | m-D9 | H | A-28 | H | tBu | A-28 | H | m-D9 | H |
| 45243 | 1 | H | m-D9 | H | A-29 | H | tBu | A-29 | H | m-D9 | H |
| 45244 | 1 | H | m-D9 | H | A-30 | H | tBu | A-30 | H | m-D9 | H |
| 45245 | 1 | H | m-D9 | H | A-31 | H | tBu | A-31 | H | m-D9 | H |
| 45246 | 1 | H | m-D9 | H | A-32 | H | tBu | A-32 | H | m-D9 | H |
| 45247 | 1 | H | m-D9 | H | A-33 | H | tBu | A-33 | H | m-D9 | H |
| 45248 | 1 | H | m-D9 | H | A-34 | H | tBu | A-34 | H | m-D9 | H |
| 45249 | 1 | H | m-D9 | H | A-35 | H | tBu | A-35 | H | m-D9 | H |
| 45250 | 1 | H | m-D9 | H | A-36 | H | tBu | A-36 | H | m-D9 | H |
| 45251 | 1 | H | m-D9 | H | A-37 | H | tBu | A-37 | H | m-D9 | H |
| 45252 | 1 | H | m-D9 | H | A-38 | H | tBu | A-38 | H | m-D9 | H |
| 45253 | 1 | H | m-D9 | H | A-39 | H | tBu | A-39 | H | m-D9 | H |
| 45254 | 1 | H | m-D9 | H | A-40 | H | tBu | A-40 | H | m-D9 | H |
| 45255 | 1 | H | m-D9 | H | A-41 | H | tBu | A-41 | H | m-D9 | H |
| 45256 | 1 | H | m-D9 | H | A-42 | H | tBu | A-42 | H | m-D9 | H |
| 45257 | 1 | H | m-D9 | H | A-43 | H | tBu | A-43 | H | m-D9 | H |
| 45258 | 1 | H | m-D9 | H | A-44 | H | tBu | A-44 | H | m-D9 | H |
| 45259 | 1 | H | m-D9 | H | A-45 | H | tBu | A-45 | H | m-D9 | H |
| 45260 | 1 | H | m-D9 | H | A-46 | H | tBu | A-46 | H | m-D9 | H |
| 45261 | 1 | H | m-D9 | H | A-47 | H | tBu | A-47 | H | m-D9 | H |
| 45262 | 1 | H | m-D10 | H | A-1 | H | tBu | A-1 | H | m-D10 | H |
| 45263 | 1 | H | m-D10 | H | A-2 | H | tBu | A-2 | H | m-D10 | H |
| 45264 | 1 | H | m-D10 | H | A-3 | H | tBu | A-3 | H | m-D10 | H |
| 45265 | 1 | H | m-D10 | H | A-4 | H | tBu | A-4 | H | m-D10 | H |
| 45266 | 1 | H | m-D10 | H | A-5 | H | tBu | A-5 | H | m-D10 | H |
| 45267 | 1 | H | m-D10 | H | A-6 | H | tBu | A-6 | H | m-D10 | H |
| 45268 | 1 | H | m-D10 | H | A-7 | H | tBu | A-7 | H | m-D10 | H |
| 45269 | 1 | H | m-D10 | H | A-8 | H | tBu | A-8 | H | m-D10 | H |
| 45270 | 1 | H | m-D10 | H | A-9 | H | tBu | A-9 | H | m-D10 | H |
| 45271 | 1 | H | m-D10 | H | A-10 | H | tBu | A-10 | H | m-D10 | H |
| 45272 | 1 | H | m-D10 | H | A-11 | H | tBu | A-11 | H | m-D10 | H |
| 45273 | 1 | H | m-D10 | H | A-12 | H | tBu | A-12 | H | m-D10 | H |
| 45274 | 1 | H | m-D10 | H | A-13 | H | tBu | A-13 | H | m-D10 | H |
| 45275 | 1 | H | m-D10 | H | A-14 | H | tBu | A-14 | H | m-D10 | H |
| 45276 | 1 | H | m-D10 | H | A-15 | H | tBu | A-15 | H | m-D10 | H |
| 45277 | 1 | H | m-D10 | H | A-16 | H | tBu | A-16 | H | m-D10 | H |
| 45278 | 1 | H | m-D10 | H | A-17 | H | tBu | A-17 | H | m-D10 | H |
| 45279 | 1 | H | m-D10 | H | A-18 | H | tBu | A-18 | H | m-D10 | H |
| 45280 | 1 | H | m-D10 | H | A-19 | H | tBu | A-19 | H | m-D10 | H |
| 45281 | 1 | H | m-D10 | H | A-20 | H | tBu | A-20 | H | m-D10 | H |
| 45282 | 1 | H | m-D10 | H | A-21 | H | tBu | A-21 | H | m-D10 | H |
| 45283 | 1 | H | m-D10 | H | A-22 | H | tBu | A-22 | H | m-D10 | H |
| 45284 | 1 | H | m-D10 | H | A-23 | H | tBu | A-23 | H | m-D10 | H |
| 45285 | 1 | H | m-D10 | H | A-24 | H | tBu | A-24 | H | m-D10 | H |
| 45286 | 1 | H | m-D10 | H | A-25 | H | tBu | A-25 | H | m-D10 | H |
| 45287 | 1 | H | m-D10 | H | A-26 | H | tBu | A-26 | H | m-D10 | H |
| 45288 | 1 | H | m-D10 | H | A-27 | H | tBu | A-27 | H | m-D10 | H |
| 45289 | 1 | H | m-D10 | H | A-28 | H | tBu | A-28 | H | m-D10 | H |
| 45290 | 1 | H | m-D10 | H | A-29 | H | tBu | A-29 | H | m-D10 | H |
| 45291 | 1 | H | m-D10 | H | A-30 | H | tBu | A-30 | H | m-D10 | H |
| 45292 | 1 | H | m-D10 | H | A-31 | H | tBu | A-31 | H | m-D10 | H |
| 45293 | 1 | H | m-D10 | H | A-32 | H | tBu | A-32 | H | m-D10 | H |
| 45294 | 1 | H | m-D10 | H | A-33 | H | tBu | A-33 | H | m-D10 | H |
| 45295 | 1 | H | m-D10 | H | A-34 | H | tBu | A-34 | H | m-D10 | H |
| 45296 | 1 | H | m-D10 | H | A-35 | H | tBu | A-35 | H | m-D10 | H |
| 45297 | 1 | H | m-D10 | H | A-36 | H | tBu | A-36 | H | m-D10 | H |
| 45298 | 1 | H | m-D10 | H | A-37 | H | tBu | A-37 | H | m-D10 | H |
| 45299 | 1 | H | m-D10 | H | A-38 | H | tBu | A-38 | H | m-D10 | H |
| 45300 | 1 | H | m-D10 | H | A-39 | H | tBu | A-39 | H | m-D10 | H |
| 45301 | 1 | H | m-D10 | H | A-40 | H | tBu | A-40 | H | m-D10 | H |
| 45302 | 1 | H | m-D10 | H | A-41 | H | tBu | A-41 | H | m-D10 | H |
| 45303 | 1 | H | m-D10 | H | A-42 | H | tBu | A-42 | H | m-D10 | H |
| 45304 | 1 | H | m-D10 | H | A-43 | H | tBu | A-43 | H | m-D10 | H |
| 45305 | 1 | H | m-D10 | H | A-44 | H | tBu | A-44 | H | m-D10 | H |
| 45306 | 1 | H | m-D10 | H | A-45 | H | tBu | A-45 | H | m-D10 | H |
| 45307 | 1 | H | m-D10 | H | A-46 | H | tBu | A-46 | H | m-D10 | H |
| 45308 | 1 | H | m-D10 | H | A-47 | H | tBu | A-47 | H | m-D10 | H |
| 45309 | 1 | H | m-D11 | H | A-1 | H | tBu | A-1 | H | m-D11 | H |
| 45310 | 1 | H | m-D11 | H | A-2 | H | tBu | A-2 | H | m-D11 | H |
| 45311 | 1 | H | m-D11 | H | A-3 | H | tBu | A-3 | H | m-D11 | H |
| 45312 | 1 | H | m-D11 | H | A-4 | H | tBu | A-4 | H | m-D11 | H |
| 45313 | 1 | H | m-D11 | H | A-5 | H | tBu | A-5 | H | m-D11 | H |
| 45314 | 1 | H | m-D11 | H | A-6 | H | tBu | A-6 | H | m-D11 | H |
| 45315 | 1 | H | m-D11 | H | A-7 | H | tBu | A-7 | H | m-D11 | H |
| 45316 | 1 | H | m-D11 | H | A-8 | H | tBu | A-8 | H | m-D11 | H |
| 45317 | 1 | H | m-D11 | H | A-9 | H | tBu | A-9 | H | m-D11 | H |
| 45318 | 1 | H | m-D11 | H | A-10 | H | tBu | A-10 | H | m-D11 | H |
| 45319 | 1 | H | m-D11 | H | A-11 | H | tBu | A-11 | H | m-D11 | H |
| 45320 | 1 | H | m-D11 | H | A-12 | H | tBu | A-12 | H | m-D11 | H |
| 45321 | 1 | H | m-D11 | H | A-13 | H | tBu | A-13 | H | m-D11 | H |
| 45322 | 1 | H | m-D11 | H | A-14 | H | tBu | A-14 | H | m-D11 | H |
| 45323 | 1 | H | m-D11 | H | A-15 | H | tBu | A-15 | H | m-D11 | H |
| 45324 | 1 | H | m-D11 | H | A-16 | H | tBu | A-16 | H | m-D11 | H |
| 45325 | 1 | H | m-D11 | H | A-17 | H | tBu | A-17 | H | m-D11 | H |
| 45326 | 1 | H | m-D11 | H | A-18 | H | tBu | A-18 | H | m-D11 | H |
| 45327 | 1 | H | m-D11 | H | A-19 | H | tBu | A-19 | H | m-D11 | H |
| 45328 | 1 | H | m-D11 | H | A-20 | H | tBu | A-20 | H | m-D11 | H |
| 45329 | 1 | H | m-D11 | H | A-21 | H | tBu | A-21 | H | m-D11 | H |
| 45330 | 1 | H | m-D11 | H | A-22 | H | tBu | A-22 | H | m-D11 | H |
| 45331 | 1 | H | m-D11 | H | A-23 | H | tBu | A-23 | H | m-D11 | H |
| 45332 | 1 | H | m-D11 | H | A-24 | H | tBu | A-24 | H | m-D11 | H |
| 45333 | 1 | H | m-D11 | H | A-25 | H | tBu | A-25 | H | m-D11 | H |
| 45334 | 1 | H | m-D11 | H | A-26 | H | tBu | A-26 | H | m-D11 | H |
| 45335 | 1 | H | m-D11 | H | A-27 | H | tBu | A-27 | H | m-D11 | H |
| 45336 | 1 | H | m-D11 | H | A-28 | H | tBu | A-28 | H | m-D11 | H |
| 45337 | 1 | H | m-D11 | H | A-29 | H | tBu | A-29 | H | m-D11 | H |
| 45338 | 1 | H | m-D11 | H | A-30 | H | tBu | A-30 | H | m-D11 | H |
| 45339 | 1 | H | m-D11 | H | A-31 | H | tBu | A-31 | H | m-D11 | H |
| 45340 | 1 | H | m-D11 | H | A-32 | H | tBu | A-32 | H | m-D11 | H |
| 45341 | 1 | H | m-D11 | H | A-33 | H | tBu | A-33 | H | m-D11 | H |
| 45342 | 1 | H | m-D11 | H | A-34 | H | tBu | A-34 | H | m-D11 | H |
| 45343 | 1 | H | m-D11 | H | A-35 | H | tBu | A-35 | H | m-D11 | H |
| 45344 | 1 | H | m-D11 | H | A-36 | H | tBu | A-36 | H | m-D11 | H |
| 45345 | 1 | H | m-D11 | H | A-37 | H | tBu | A-37 | H | m-D11 | H |
| 45346 | 1 | H | m-D11 | H | A-38 | H | tBu | A-38 | H | m-D11 | H |
| 45347 | 1 | H | m-D11 | H | A-39 | H | tBu | A-39 | H | m-D11 | H |
| 45348 | 1 | H | m-D11 | H | A-40 | H | tBu | A-40 | H | m-D11 | H |
| 45349 | 1 | H | m-D11 | H | A-41 | H | tBu | A-41 | H | m-D11 | H |
| 45350 | 1 | H | m-D11 | H | A-42 | H | tBu | A-42 | H | m-D11 | H |
| 45351 | 1 | H | m-D11 | H | A-43 | H | tBu | A-43 | H | m-D11 | H |
| 45352 | 1 | H | m-D11 | H | A-44 | H | tBu | A-44 | H | m-D11 | H |
| 45353 | 1 | H | m-D11 | H | A-45 | H | tBu | A-45 | H | m-D11 | H |
| 45354 | 1 | H | m-D11 | H | A-46 | H | tBu | A-46 | H | m-D11 | H |
| 45355 | 1 | H | m-D11 | H | A-47 | H | tBu | A-47 | H | m-D11 | H |
| 45356 | 1 | H | m-D12 | H | A-1 | H | tBu | A-1 | H | m-D12 | H |
| 45357 | 1 | H | m-D12 | H | A-2 | H | tBu | A-2 | H | m-D12 | H |
| 45358 | 1 | H | m-D12 | H | A-3 | H | tBu | A-3 | H | m-D12 | H |
| 45359 | 1 | H | m-D12 | H | A-4 | H | tBu | A-4 | H | m-D12 | H |
| 45360 | 1 | H | m-D12 | H | A-5 | H | tBu | A-5 | H | m-D12 | H |
| 45361 | 1 | H | m-D12 | H | A-6 | H | tBu | A-6 | H | m-D12 | H |
| 45362 | 1 | H | m-D12 | H | A-7 | H | tBu | A-7 | H | m-D12 | H |
| 45363 | 1 | H | m-D12 | H | A-8 | H | tBu | A-8 | H | m-D12 | H |
| 45364 | 1 | H | m-D12 | H | A-9 | H | tBu | A-9 | H | m-D12 | H |
| 45365 | 1 | H | m-D12 | H | A-10 | H | tBu | A-10 | H | m-D12 | H |
| 45366 | 1 | H | m-D12 | H | A-11 | H | tBu | A-11 | H | m-D12 | H |
| 45367 | 1 | H | m-D12 | H | A-12 | H | tBu | A-12 | H | m-D12 | H |
| 45368 | 1 | H | m-D12 | H | A-13 | H | tBu | A-13 | H | m-D12 | H |
| 45369 | 1 | H | m-D12 | H | A-14 | H | tBu | A-14 | H | m-D12 | H |
| 45370 | 1 | H | m-D12 | H | A-15 | H | tBu | A-15 | H | m-D12 | H |
| 45371 | 1 | H | m-D12 | H | A-16 | H | tBu | A-16 | H | m-D12 | H |
| 45372 | 1 | H | m-D12 | H | A-17 | H | tBu | A-17 | H | m-D12 | H |
| 45373 | 1 | H | m-D12 | H | A-18 | H | tBu | A-18 | H | m-D12 | H |
| 45374 | 1 | H | m-D12 | H | A-19 | H | tBu | A-19 | H | m-D12 | H |
| 45375 | 1 | H | m-D12 | H | A-20 | H | tBu | A-20 | H | m-D12 | H |
| 45376 | 1 | H | m-D12 | H | A-21 | H | tBu | A-21 | H | m-D12 | H |
| 45377 | 1 | H | m-D12 | H | A-22 | H | tBu | A-22 | H | m-D12 | H |
| 45378 | 1 | H | m-D12 | H | A-23 | H | tBu | A-23 | H | m-D12 | H |
| 45379 | 1 | H | m-D12 | H | A-24 | H | tBu | A-24 | H | m-D12 | H |
| 45380 | 1 | H | m-D12 | H | A-25 | H | tBu | A-25 | H | m-D12 | H |
| 45381 | 1 | H | m-D12 | H | A-26 | H | tBu | A-26 | H | m-D12 | H |
| 45382 | 1 | H | m-D12 | H | A-27 | H | tBu | A-27 | H | m-D12 | H |
| 45383 | 1 | H | m-D12 | H | A-28 | H | tBu | A-28 | H | m-D12 | H |
| 45384 | 1 | H | m-D12 | H | A-29 | H | tBu | A-29 | H | m-D12 | H |
| 45385 | 1 | H | m-D12 | H | A-30 | H | tBu | A-30 | H | m-D12 | H |
| 45386 | 1 | H | m-D12 | H | A-31 | H | tBu | A-31 | H | m-D12 | H |
| 45387 | 1 | H | m-D12 | H | A-32 | H | tBu | A-32 | H | m-D12 | H |
| 45388 | 1 | H | m-D12 | H | A-33 | H | tBu | A-33 | H | m-D12 | H |
| 45389 | 1 | H | m-D12 | H | A-34 | H | tBu | A-34 | H | m-D12 | H |
| 45390 | 1 | H | m-D12 | H | A-35 | H | tBu | A-35 | H | m-D12 | H |
| 45391 | 1 | H | m-D12 | H | A-36 | H | tBu | A-36 | H | m-D12 | H |
| 45392 | 1 | H | m-D12 | H | A-37 | H | tBu | A-37 | H | m-D12 | H |
| 45393 | 1 | H | m-D12 | H | A-38 | H | tBu | A-38 | H | m-D12 | H |
| 45394 | 1 | H | m-D12 | H | A-39 | H | tBu | A-39 | H | m-D12 | H |
| 45395 | 1 | H | m-D12 | H | A-40 | H | tBu | A-40 | H | m-D12 | H |
| 45396 | 1 | H | m-D12 | H | A-41 | H | tBu | A-41 | H | m-D12 | H |
| 45397 | 1 | H | m-D12 | H | A-42 | H | tBu | A-42 | H | m-D12 | H |
| 45398 | 1 | H | m-D12 | H | A-43 | H | tBu | A-43 | H | m-D12 | H |
| 45399 | 1 | H | m-D12 | H | A-44 | H | tBu | A-44 | H | m-D12 | H |
| 45400 | 1 | H | m-D12 | H | A-45 | H | tBu | A-45 | H | m-D12 | H |
| 45401 | 1 | H | m-D12 | H | A-46 | H | tBu | A-46 | H | m-D12 | H |
| 45402 | 1 | H | m-D12 | H | A-47 | H | tBu | A-47 | H | m-D12 | H |
| 45403 | 1 | H | m-D13 | H | A-1 | H | tBu | A-1 | H | m-D13 | H |
| 45404 | 1 | H | m-D13 | H | A-2 | H | tBu | A-2 | H | m-D13 | H |
| 45405 | 1 | H | m-D13 | H | A-3 | H | tBu | A-3 | H | m-D13 | H |
| 45406 | 1 | H | m-D13 | H | A-4 | H | tBu | A-4 | H | m-D13 | H |
| 45407 | 1 | H | m-D13 | H | A-5 | H | tBu | A-5 | H | m-D13 | H |
| 45408 | 1 | H | m-D13 | H | A-6 | H | tBu | A-6 | H | m-D13 | H |
| 45409 | 1 | H | m-D13 | H | A-7 | H | tBu | A-7 | H | m-D13 | H |
| 45410 | 1 | H | m-D13 | H | A-8 | H | tBu | A-8 | H | m-D13 | H |
| 45411 | 1 | H | m-D13 | H | A-9 | H | tBu | A-9 | H | m-D13 | H |
| 45412 | 1 | H | m-D13 | H | A-10 | H | tBu | A-10 | H | m-D13 | H |
| 45413 | 1 | H | m-D13 | H | A-11 | H | tBu | A-11 | H | m-D13 | H |
| 45414 | 1 | H | m-D13 | H | A-12 | H | tBu | A-12 | H | m-D13 | H |
| 45415 | 1 | H | m-D13 | H | A-13 | H | tBu | A-13 | H | m-D13 | H |
| 45416 | 1 | H | m-D13 | H | A-14 | H | tBu | A-14 | H | m-D13 | H |
| 45417 | 1 | H | m-D13 | H | A-15 | H | tBu | A-15 | H | m-D13 | H |
| 45418 | 1 | H | m-D13 | H | A-16 | H | tBu | A-16 | H | m-D13 | H |
| 45419 | 1 | H | m-D13 | H | A-17 | H | tBu | A-17 | H | m-D13 | H |
| 45420 | 1 | H | m-D13 | H | A-18 | H | tBu | A-18 | H | m-D13 | H |
| 45421 | 1 | H | m-D13 | H | A-19 | H | tBu | A-19 | H | m-D13 | H |
| 45422 | 1 | H | m-D13 | H | A-20 | H | tBu | A-20 | H | m-D13 | H |
| 45423 | 1 | H | m-D13 | H | A-21 | H | tBu | A-21 | H | m-D13 | H |
| 45424 | 1 | H | m-D13 | H | A-22 | H | tBu | A-22 | H | m-D13 | H |
| 45425 | 1 | H | m-D13 | H | A-23 | H | tBu | A-23 | H | m-D13 | H |
| 45426 | 1 | H | m-D13 | H | A-24 | H | tBu | A-24 | H | m-D13 | H |
| 45427 | 1 | H | m-D13 | H | A-25 | H | tBu | A-25 | H | m-D13 | H |
| 45428 | 1 | H | m-D13 | H | A-26 | H | tBu | A-26 | H | m-D13 | H |
| 45429 | 1 | H | m-D13 | H | A-27 | H | tBu | A-27 | H | m-D13 | H |
| 45430 | 1 | H | m-D13 | H | A-28 | H | tBu | A-28 | H | m-D13 | H |
| 45431 | 1 | H | m-D13 | H | A-29 | H | tBu | A-29 | H | m-D13 | H |
| 45432 | 1 | H | m-D13 | H | A-30 | H | tBu | A-30 | H | m-D13 | H |
| 45433 | 1 | H | m-D13 | H | A-31 | H | tBu | A-31 | H | m-D13 | H |
| 45434 | 1 | H | m-D13 | H | A-32 | H | tBu | A-32 | H | m-D13 | H |
| 45435 | 1 | H | m-D13 | H | A-33 | H | tBu | A-33 | H | m-D13 | H |
| 45436 | 1 | H | m-D13 | H | A-34 | H | tBu | A-34 | H | m-D13 | H |
| 45437 | 1 | H | m-D13 | H | A-35 | H | tBu | A-35 | H | m-D13 | H |
| 45438 | 1 | H | m-D13 | H | A-36 | H | tBu | A-36 | H | m-D13 | H |
| 45439 | 1 | H | m-D13 | H | A-37 | H | tBu | A-37 | H | m-D13 | H |
| 45440 | 1 | H | m-D13 | H | A-38 | H | tBu | A-38 | H | m-D13 | H |
| 45441 | 1 | H | m-D13 | H | A-39 | H | tBu | A-39 | H | m-D13 | H |
| 45442 | 1 | H | m-D13 | H | A-40 | H | tBu | A-40 | H | m-D13 | H |
| 45443 | 1 | H | m-D13 | H | A-41 | H | tBu | A-41 | H | m-D13 | H |
| 45444 | 1 | H | m-D13 | H | A-42 | H | tBu | A-42 | H | m-D13 | H |
| 45445 | 1 | H | m-D13 | H | A-43 | H | tBu | A-43 | H | m-D13 | H |
| 45446 | 1 | H | m-D13 | H | A-44 | H | tBu | A-44 | H | m-D13 | H |
| 45447 | 1 | H | m-D13 | H | A-45 | H | tBu | A-45 | H | m-D13 | H |
| 45448 | 1 | H | m-D13 | H | A-46 | H | tBu | A-46 | H | m-D13 | H |
| 45449 | 1 | H | m-D13 | H | A-47 | H | tBu | A-47 | H | m-D13 | H |
| 45450 | 1 | H | m-D14 | H | A-1 | H | tBu | A-1 | H | m-D14 | H |
| 45451 | 1 | H | m-D14 | H | A-2 | H | tBu | A-2 | H | m-D14 | H |
| 45452 | 1 | H | m-D14 | H | A-3 | H | tBu | A-3 | H | m-D14 | H |
| 45453 | 1 | H | m-D14 | H | A-4 | H | tBu | A-4 | H | m-D14 | H |
| 45454 | 1 | H | m-D14 | H | A-5 | H | tBu | A-5 | H | m-D14 | H |
| 45455 | 1 | H | m-D14 | H | A-6 | H | tBu | A-6 | H | m-D14 | H |
| 45456 | 1 | H | m-D14 | H | A-7 | H | tBu | A-7 | H | m-D14 | H |
| 45457 | 1 | H | m-D14 | H | A-8 | H | tBu | A-8 | H | m-D14 | H |
| 45458 | 1 | H | m-D14 | H | A-9 | H | tBu | A-9 | H | m-D14 | H |
| 45459 | 1 | H | m-D14 | H | A-10 | H | tBu | A-10 | H | m-D14 | H |
| 45460 | 1 | H | m-D14 | H | A-11 | H | tBu | A-11 | H | m-D14 | H |
| 45461 | 1 | H | m-D14 | H | A-12 | H | tBu | A-12 | H | m-D14 | H |
| 45462 | 1 | H | m-D14 | H | A-13 | H | tBu | A-13 | H | m-D14 | H |
| 45463 | 1 | H | m-D14 | H | A-14 | H | tBu | A-14 | H | m-D14 | H |
| 45464 | 1 | H | m-D14 | H | A-15 | H | tBu | A-15 | H | m-D14 | H |
| 45465 | 1 | H | m-D14 | H | A-16 | H | tBu | A-16 | H | m-D14 | H |
| 45466 | 1 | H | m-D14 | H | A-17 | H | tBu | A-17 | H | m-D14 | H |
| 45467 | 1 | H | m-D14 | H | A-18 | H | tBu | A-18 | H | m-D14 | H |
| 45468 | 1 | H | m-D14 | H | A-19 | H | tBu | A-19 | H | m-D14 | H |
| 45469 | 1 | H | m-D14 | H | A-20 | H | tBu | A-20 | H | m-D14 | H |
| 45470 | 1 | H | m-D14 | H | A-21 | H | tBu | A-21 | H | m-D14 | H |
| 45471 | 1 | H | m-D14 | H | A-22 | H | tBu | A-22 | H | m-D14 | H |
| 45472 | 1 | H | m-D14 | H | A-23 | H | tBu | A-23 | H | m-D14 | H |
| 45473 | 1 | H | m-D14 | H | A-24 | H | tBu | A-24 | H | m-D14 | H |
| 45474 | 1 | H | m-D14 | H | A-25 | H | tBu | A-25 | H | m-D14 | H |
| 45475 | 1 | H | m-D14 | H | A-26 | H | tBu | A-26 | H | m-D14 | H |
| 45476 | 1 | H | m-D14 | H | A-27 | H | tBu | A-27 | H | m-D14 | H |
| 45477 | 1 | H | m-D14 | H | A-28 | H | tBu | A-28 | H | m-D14 | H |
| 45478 | 1 | H | m-D14 | H | A-29 | H | tBu | A-29 | H | m-D14 | H |
| 45479 | 1 | H | m-D14 | H | A-30 | H | tBu | A-30 | H | m-D14 | H |
| 45480 | 1 | H | m-D14 | H | A-31 | H | tBu | A-31 | H | m-D14 | H |
| 45481 | 1 | H | m-D14 | H | A-32 | H | tBu | A-32 | H | m-D14 | H |
| 45482 | 1 | H | m-D14 | H | A-33 | H | tBu | A-33 | H | m-D14 | H |
| 45483 | 1 | H | m-D14 | H | A-34 | H | tBu | A-34 | H | m-D14 | H |
| 45484 | 1 | H | m-D14 | H | A-35 | H | tBu | A-35 | H | m-D14 | H |
| 45485 | 1 | H | m-D14 | H | A-36 | H | tBu | A-36 | H | m-D14 | H |
| 45486 | 1 | H | m-D14 | H | A-37 | H | tBu | A-37 | H | m-D14 | H |
| 45487 | 1 | H | m-D14 | H | A-38 | H | tBu | A-38 | H | m-D14 | H |
| 45488 | 1 | H | m-D14 | H | A-39 | H | tBu | A-39 | H | m-D14 | H |
| 45489 | 1 | H | m-D14 | H | A-40 | H | tBu | A-40 | H | m-D14 | H |
| 45490 | 1 | H | m-D14 | H | A-41 | H | tBu | A-41 | H | m-D14 | H |
| 45491 | 1 | H | m-D14 | H | A-42 | H | tBu | A-42 | H | m-D14 | H |
| 45492 | 1 | H | m-D14 | H | A-43 | H | tBu | A-43 | H | m-D14 | H |
| 45493 | 1 | H | m-D14 | H | A-44 | H | tBu | A-44 | H | m-D14 | H |
| 45494 | 1 | H | m-D14 | H | A-45 | H | tBu | A-45 | H | m-D14 | H |
| 45495 | 1 | H | m-D14 | H | A-46 | H | tBu | A-46 | H | m-D14 | H |
| 45496 | 1 | H | m-D14 | H | A-47 | H | tBu | A-47 | H | m-D14 | H |
| 45497 | 1 | H | m-D1 | H | A-1 | H | tBu | m-D1 | H | A-1 | H |
| 45498 | 1 | H | m-D1 | H | A-2 | H | tBu | m-D1 | H | A-2 | H |
| 45499 | 1 | H | m-D1 | H | A-3 | H | tBu | m-D1 | H | A-3 | H |
| 45500 | 1 | H | m-D1 | H | A-4 | H | tBu | m-D1 | H | A-4 | H |
| 45501 | 1 | H | m-D1 | H | A-5 | H | tBu | m-D1 | H | A-5 | H |
| 45502 | 1 | H | m-D1 | H | A-6 | H | tBu | m-D1 | H | A-6 | H |
| 45503 | 1 | H | m-D1 | H | A-7 | H | tBu | m-D1 | H | A-7 | H |
| 45504 | 1 | H | m-D1 | H | A-8 | H | tBu | m-D1 | H | A-8 | H |
| 45505 | 1 | H | m-D1 | H | A-9 | H | tBu | m-D1 | H | A-9 | H |
| 45506 | 1 | H | m-D1 | H | A-10 | H | tBu | m-D1 | H | A-10 | H |
| 45507 | 1 | H | m-D1 | H | A-11 | H | tBu | m-D1 | H | A-11 | H |
| 45508 | 1 | H | m-D1 | H | A-12 | H | tBu | m-D1 | H | A-12 | H |
| 45509 | 1 | H | m-D1 | H | A-13 | H | tBu | m-D1 | H | A-13 | H |
| 45510 | 1 | H | m-D1 | H | A-14 | H | tBu | m-D1 | H | A-14 | H |
| 45511 | 1 | H | m-D1 | H | A-15 | H | tBu | m-D1 | H | A-15 | H |
| 45512 | 1 | H | m-D1 | H | A-16 | H | tBu | m-D1 | H | A-16 | H |
| 45513 | 1 | H | m-D1 | H | A-17 | H | tBu | m-D1 | H | A-17 | H |
| 45514 | 1 | H | m-D1 | H | A-18 | H | tBu | m-D1 | H | A-18 | H |
| 45515 | 1 | H | m-D1 | H | A-19 | H | tBu | m-D1 | H | A-19 | H |
| 45516 | 1 | H | m-D1 | H | A-20 | H | tBu | m-D1 | H | A-20 | H |
| 45517 | 1 | H | m-D1 | H | A-21 | H | tBu | m-D1 | H | A-21 | H |
| 45518 | 1 | H | m-D1 | H | A-22 | H | tBu | m-D1 | H | A-22 | H |
| 45519 | 1 | H | m-D1 | H | A-23 | H | tBu | m-D1 | H | A-23 | H |
| 45520 | 1 | H | m-D1 | H | A-24 | H | tBu | m-D1 | H | A-24 | H |
| 45521 | 1 | H | m-D1 | H | A-25 | H | tBu | m-D1 | H | A-25 | H |
| 45522 | 1 | H | m-D1 | H | A-26 | H | tBu | m-D1 | H | A-26 | H |
| 45523 | 1 | H | m-D1 | H | A-27 | H | tBu | m-D1 | H | A-27 | H |
| 45524 | 1 | H | m-D1 | H | A-28 | H | tBu | m-D1 | H | A-28 | H |
| 45525 | 1 | H | m-D1 | H | A-29 | H | tBu | m-D1 | H | A-29 | H |
| 45526 | 1 | H | m-D1 | H | A-30 | H | tBu | m-D1 | H | A-30 | H |
| 45527 | 1 | H | m-D1 | H | A-31 | H | tBu | m-D1 | H | A-31 | H |
| 45528 | 1 | H | m-D1 | H | A-32 | H | tBu | m-D1 | H | A-32 | H |
| 45529 | 1 | H | m-D1 | H | A-33 | H | tBu | m-D1 | H | A-33 | H |
| 45530 | 1 | H | m-D1 | H | A-34 | H | tBu | m-D1 | H | A-34 | H |
| 45531 | 1 | H | m-D1 | H | A-35 | H | tBu | m-D1 | H | A-35 | H |
| 45532 | 1 | H | m-D1 | H | A-36 | H | tBu | m-D1 | H | A-36 | H |
| 45533 | 1 | H | m-D1 | H | A-37 | H | tBu | m-D1 | H | A-37 | H |
| 45534 | 1 | H | m-D1 | H | A-38 | H | tBu | m-D1 | H | A-38 | H |
| 45535 | 1 | H | m-D1 | H | A-39 | H | tBu | m-D1 | H | A-39 | H |
| 45536 | 1 | H | m-D1 | H | A-40 | H | tBu | m-D1 | H | A-40 | H |
| 45537 | 1 | H | m-D1 | H | A-41 | H | tBu | m-D1 | H | A-41 | H |
| 45538 | 1 | H | m-D1 | H | A-42 | H | tBu | m-D1 | H | A-42 | H |
| 45539 | 1 | H | m-D1 | H | A-43 | H | tBu | m-D1 | H | A-43 | H |
| 45540 | 1 | H | m-D1 | H | A-44 | H | tBu | m-D1 | H | A-44 | H |
| 45541 | 1 | H | m-D1 | H | A-45 | H | tBu | m-D1 | H | A-45 | H |
| 45542 | 1 | H | m-D1 | H | A-46 | H | tBu | m-D1 | H | A-46 | H |
| 45543 | 1 | H | m-D1 | H | A-47 | H | tBu | m-D1 | H | A-47 | H |
| 45544 | 1 | H | m-D2 | H | A-1 | H | tBu | m-D2 | H | A-1 | H |
| 45545 | 1 | H | m-D2 | H | A-2 | H | tBu | m-D2 | H | A-2 | H |
| 45546 | 1 | H | m-D2 | H | A-3 | H | tBu | m-D2 | H | A-3 | H |
| 45547 | 1 | H | m-D2 | H | A-4 | H | tBu | m-D2 | H | A-4 | H |
| 45548 | 1 | H | m-D2 | H | A-5 | H | tBu | m-D2 | H | A-5 | H |
| 45549 | 1 | H | m-D2 | H | A-6 | H | tBu | m-D2 | H | A-6 | H |
| 45550 | 1 | H | m-D2 | H | A-7 | H | tBu | m-D2 | H | A-7 | H |
| 45551 | 1 | H | m-D2 | H | A-8 | H | tBu | m-D2 | H | A-8 | H |
| 45552 | 1 | H | m-D2 | H | A-9 | H | tBu | m-D2 | H | A-9 | H |
| 45553 | 1 | H | m-D2 | H | A-10 | H | tBu | m-D2 | H | A-10 | H |
| 45554 | 1 | H | m-D2 | H | A-11 | H | tBu | m-D2 | H | A-11 | H |
| 45555 | 1 | H | m-D2 | H | A-12 | H | tBu | m-D2 | H | A-12 | H |
| 45556 | 1 | H | m-D2 | H | A-13 | H | tBu | m-D2 | H | A-13 | H |
| 45557 | 1 | H | m-D2 | H | A-14 | H | tBu | m-D2 | H | A-14 | H |
| 45558 | 1 | H | m-D2 | H | A-15 | H | tBu | m-D2 | H | A-15 | H |
| 45559 | 1 | H | m-D2 | H | A-16 | H | tBu | m-D2 | H | A-16 | H |
| 45560 | 1 | H | m-D2 | H | A-17 | H | tBu | m-D2 | H | A-17 | H |
| 45561 | 1 | H | m-D2 | H | A-18 | H | tBu | m-D2 | H | A-18 | H |
| 45562 | 1 | H | m-D2 | H | A-19 | H | tBu | m-D2 | H | A-19 | H |
| 45563 | 1 | H | m-D2 | H | A-20 | H | tBu | m-D2 | H | A-20 | H |
| 45564 | 1 | H | m-D2 | H | A-21 | H | tBu | m-D2 | H | A-21 | H |
| 45565 | 1 | H | m-D2 | H | A-22 | H | tBu | m-D2 | H | A-22 | H |
| 45566 | 1 | H | m-D2 | H | A-23 | H | tBu | m-D2 | H | A-23 | H |
| 45567 | 1 | H | m-D2 | H | A-24 | H | tBu | m-D2 | H | A-24 | H |
| 45568 | 1 | H | m-D2 | H | A-25 | H | tBu | m-D2 | H | A-25 | H |
| 45569 | 1 | H | m-D2 | H | A-26 | H | tBu | m-D2 | H | A-26 | H |
| 45570 | 1 | H | m-D2 | H | A-27 | H | tBu | m-D2 | H | A-27 | H |
| 45571 | 1 | H | m-D2 | H | A-28 | H | tBu | m-D2 | H | A-28 | H |
| 45572 | 1 | H | m-D2 | H | A-29 | H | tBu | m-D2 | H | A-29 | H |
| 45573 | 1 | H | m-D2 | H | A-30 | H | tBu | m-D2 | H | A-30 | H |
| 45574 | 1 | H | m-D2 | H | A-31 | H | tBu | m-D2 | H | A-31 | H |
| 45575 | 1 | H | m-D2 | H | A-32 | H | tBu | m-D2 | H | A-32 | H |
| 45576 | 1 | H | m-D2 | H | A-33 | H | tBu | m-D2 | H | A-33 | H |
| 45577 | 1 | H | m-D2 | H | A-34 | H | tBu | m-D2 | H | A-34 | H |
| 45578 | 1 | H | m-D2 | H | A-35 | H | tBu | m-D2 | H | A-35 | H |
| 45579 | 1 | H | m-D2 | H | A-36 | H | tBu | m-D2 | H | A-36 | H |
| 45580 | 1 | H | m-D2 | H | A-37 | H | tBu | m-D2 | H | A-37 | H |
| 45581 | 1 | H | m-D2 | H | A-38 | H | tBu | m-D2 | H | A-38 | H |
| 45582 | 1 | H | m-D2 | H | A-39 | H | tBu | m-D2 | H | A-39 | H |
| 45583 | 1 | H | m-D2 | H | A-40 | H | tBu | m-D2 | H | A-40 | H |
| 45584 | 1 | H | m-D2 | H | A-41 | H | tBu | m-D2 | H | A-41 | H |
| 45585 | 1 | H | m-D2 | H | A-42 | H | tBu | m-D2 | H | A-42 | H |
| 45586 | 1 | H | m-D2 | H | A-43 | H | tBu | m-D2 | H | A-43 | H |
| 45587 | 1 | H | m-D2 | H | A-44 | H | tBu | m-D2 | H | A-44 | H |
| 45588 | 1 | H | m-D2 | H | A-45 | H | tBu | m-D2 | H | A-45 | H |
| 45589 | 1 | H | m-D2 | H | A-46 | H | tBu | m-D2 | H | A-46 | H |
| 45590 | 1 | H | m-D2 | H | A-47 | H | tBu | m-D2 | H | A-47 | H |
| 45591 | 1 | H | m-D3 | H | A-1 | H | tBu | m-D3 | H | A-1 | H |
| 45592 | 1 | H | m-D3 | H | A-2 | H | tBu | m-D3 | H | A-2 | H |
| 45593 | 1 | H | m-D3 | H | A-3 | H | tBu | m-D3 | H | A-3 | H |
| 45594 | 1 | H | m-D3 | H | A-4 | H | tBu | m-D3 | H | A-4 | H |
| 45595 | 1 | H | m-D3 | H | A-5 | H | tBu | m-D3 | H | A-5 | H |
| 45596 | 1 | H | m-D3 | H | A-6 | H | tBu | m-D3 | H | A-6 | H |
| 45597 | 1 | H | m-D3 | H | A-7 | H | tBu | m-D3 | H | A-7 | H |
| 45598 | 1 | H | m-D3 | H | A-8 | H | tBu | m-D3 | H | A-8 | H |
| 45599 | 1 | H | m-D3 | H | A-9 | H | tBu | m-D3 | H | A-9 | H |
| 45600 | 1 | H | m-D3 | H | A-10 | H | tBu | m-D3 | H | A-10 | H |
| 45601 | 1 | H | m-D3 | H | A-11 | H | tBu | m-D3 | H | A-11 | H |
| 45602 | 1 | H | m-D3 | H | A-12 | H | tBu | m-D3 | H | A-12 | H |
| 45603 | 1 | H | m-D3 | H | A-13 | H | tBu | m-D3 | H | A-13 | H |
| 45604 | 1 | H | m-D3 | H | A-14 | H | tBu | m-D3 | H | A-14 | H |
| 45605 | 1 | H | m-D3 | H | A-15 | H | tBu | m-D3 | H | A-15 | H |
| 45606 | 1 | H | m-D3 | H | A-16 | H | tBu | m-D3 | H | A-16 | H |
| 45607 | 1 | H | m-D3 | H | A-17 | H | tBu | m-D3 | H | A-17 | H |
| 45608 | 1 | H | m-D3 | H | A-18 | H | tBu | m-D3 | H | A-18 | H |
| 45609 | 1 | H | m-D3 | H | A-19 | H | tBu | m-D3 | H | A-19 | H |
| 45610 | 1 | H | m-D3 | H | A-20 | H | tBu | m-D3 | H | A-20 | H |
| 45611 | 1 | H | m-D3 | H | A-21 | H | tBu | m-D3 | H | A-21 | H |
| 45612 | 1 | H | m-D3 | H | A-22 | H | tBu | m-D3 | H | A-22 | H |
| 45613 | 1 | H | m-D3 | H | A-23 | H | tBu | m-D3 | H | A-23 | H |
| 45614 | 1 | H | m-D3 | H | A-24 | H | tBu | m-D3 | H | A-24 | H |
| 45615 | 1 | H | m-D3 | H | A-25 | H | tBu | m-D3 | H | A-25 | H |
| 45616 | 1 | H | m-D3 | H | A-26 | H | tBu | m-D3 | H | A-26 | H |
| 45617 | 1 | H | m-D3 | H | A-27 | H | tBu | m-D3 | H | A-27 | H |
| 45618 | 1 | H | m-D3 | H | A-28 | H | tBu | m-D3 | H | A-28 | H |
| 45619 | 1 | H | m-D3 | H | A-29 | H | tBu | m-D3 | H | A-29 | H |
| 45620 | 1 | H | m-D3 | H | A-30 | H | tBu | m-D3 | H | A-30 | H |
| 45621 | 1 | H | m-D3 | H | A-31 | H | tBu | m-D3 | H | A-31 | H |
| 45622 | 1 | H | m-D3 | H | A-32 | H | tBu | m-D3 | H | A-32 | H |
| 45623 | 1 | H | m-D3 | H | A-33 | H | tBu | m-D3 | H | A-33 | H |
| 45624 | 1 | H | m-D3 | H | A-34 | H | tBu | m-D3 | H | A-34 | H |
| 45625 | 1 | H | m-D3 | H | A-35 | H | tBu | m-D3 | H | A-35 | H |
| 45626 | 1 | H | m-D3 | H | A-36 | H | tBu | m-D3 | H | A-36 | H |
| 45627 | 1 | H | m-D3 | H | A-37 | H | tBu | m-D3 | H | A-37 | H |
| 45628 | 1 | H | m-D3 | H | A-38 | H | tBu | m-D3 | H | A-38 | H |
| 45629 | 1 | H | m-D3 | H | A-39 | H | tBu | m-D3 | H | A-39 | H |
| 45630 | 1 | H | m-D3 | H | A-40 | H | tBu | m-D3 | H | A-40 | H |
| 45631 | 1 | H | m-D3 | H | A-41 | H | tBu | m-D3 | H | A-41 | H |
| 45632 | 1 | H | m-D3 | H | A-42 | H | tBu | m-D3 | H | A-42 | H |
| 45633 | 1 | H | m-D3 | H | A-43 | H | tBu | m-D3 | H | A-43 | H |
| 45634 | 1 | H | m-D3 | H | A-44 | H | tBu | m-D3 | H | A-44 | H |
| 45635 | 1 | H | m-D3 | H | A-45 | H | tBu | m-D3 | H | A-45 | H |
| 45636 | 1 | H | m-D3 | H | A-46 | H | tBu | m-D3 | H | A-46 | H |
| 45637 | 1 | H | m-D3 | H | A-47 | H | tBu | m-D3 | H | A-47 | H |
| 45638 | 1 | H | m-D4 | H | A-1 | H | tBu | m-D4 | H | A-1 | H |
| 45639 | 1 | H | m-D4 | H | A-2 | H | tBu | m-D4 | H | A-2 | H |
| 45640 | 1 | H | m-D4 | H | A-3 | H | tBu | m-D4 | H | A-3 | H |
| 45641 | 1 | H | m-D4 | H | A-4 | H | tBu | m-D4 | H | A-4 | H |
| 45642 | 1 | H | m-D4 | H | A-5 | H | tBu | m-D4 | H | A-5 | H |
| 45643 | 1 | H | m-D4 | H | A-6 | H | tBu | m-D4 | H | A-6 | H |
| 45644 | 1 | H | m-D4 | H | A-7 | H | tBu | m-D4 | H | A-7 | H |
| 45645 | 1 | H | m-D4 | H | A-8 | H | tBu | m-D4 | H | A-8 | H |
| 45646 | 1 | H | m-D4 | H | A-9 | H | tBu | m-D4 | H | A-9 | H |
| 45647 | 1 | H | m-D4 | H | A-10 | H | tBu | m-D4 | H | A-10 | H |
| 45648 | 1 | H | m-D4 | H | A-11 | H | tBu | m-D4 | H | A-11 | H |
| 45649 | 1 | H | m-D4 | H | A-12 | H | tBu | m-D4 | H | A-12 | H |
| 45650 | 1 | H | m-D4 | H | A-13 | H | tBu | m-D4 | H | A-13 | H |
| 45651 | 1 | H | m-D4 | H | A-14 | H | tBu | m-D4 | H | A-14 | H |
| 45652 | 1 | H | m-D4 | H | A-15 | H | tBu | m-D4 | H | A-15 | H |
| 45653 | 1 | H | m-D4 | H | A-16 | H | tBu | m-D4 | H | A-16 | H |
| 45654 | 1 | H | m-D4 | H | A-17 | H | tBu | m-D4 | H | A-17 | H |
| 45655 | 1 | H | m-D4 | H | A-18 | H | tBu | m-D4 | H | A-18 | H |
| 45656 | 1 | H | m-D4 | H | A-19 | H | tBu | m-D4 | H | A-19 | H |
| 45657 | 1 | H | m-D4 | H | A-20 | H | tBu | m-D4 | H | A-20 | H |
| 45658 | 1 | H | m-D4 | H | A-21 | H | tBu | m-D4 | H | A-21 | H |
| 45659 | 1 | H | m-D4 | H | A-22 | H | tBu | m-D4 | H | A-22 | H |
| 45660 | 1 | H | m-D4 | H | A-23 | H | tBu | m-D4 | H | A-23 | H |
| 45661 | 1 | H | m-D4 | H | A-24 | H | tBu | m-D4 | H | A-24 | H |
| 45662 | 1 | H | m-D4 | H | A-25 | H | tBu | m-D4 | H | A-25 | H |
| 45663 | 1 | H | m-D4 | H | A-26 | H | tBu | m-D4 | H | A-26 | H |
| 45664 | 1 | H | m-D4 | H | A-27 | H | tBu | m-D4 | H | A-27 | H |
| 45665 | 1 | H | m-D4 | H | A-28 | H | tBu | m-D4 | H | A-28 | H |
| 45666 | 1 | H | m-D4 | H | A-29 | H | tBu | m-D4 | H | A-29 | H |
| 45667 | 1 | H | m-D4 | H | A-30 | H | tBu | m-D4 | H | A-30 | H |
| 45668 | 1 | H | m-D4 | H | A-31 | H | tBu | m-D4 | H | A-31 | H |
| 45669 | 1 | H | m-D4 | H | A-32 | H | tBu | m-D4 | H | A-32 | H |
| 45670 | 1 | H | m-D4 | H | A-33 | H | tBu | m-D4 | H | A-33 | H |
| 45671 | 1 | H | m-D4 | H | A-34 | H | tBu | m-D4 | H | A-34 | H |
| 45672 | 1 | H | m-D4 | H | A-35 | H | tBu | m-D4 | H | A-35 | H |
| 45673 | 1 | H | m-D4 | H | A-36 | H | tBu | m-D4 | H | A-36 | H |
| 45674 | 1 | H | m-D4 | H | A-37 | H | tBu | m-D4 | H | A-37 | H |
| 45675 | 1 | H | m-D4 | H | A-38 | H | tBu | m-D4 | H | A-38 | H |
| 45676 | 1 | H | m-D4 | H | A-39 | H | tBu | m-D4 | H | A-39 | H |
| 45677 | 1 | H | m-D4 | H | A-40 | H | tBu | m-D4 | H | A-40 | H |
| 45678 | 1 | H | m-D4 | H | A-41 | H | tBu | m-D4 | H | A-41 | H |
| 45679 | 1 | H | m-D4 | H | A-42 | H | tBu | m-D4 | H | A-42 | H |
| 45680 | 1 | H | m-D4 | H | A-43 | H | tBu | m-D4 | H | A-43 | H |
| 45681 | 1 | H | m-D4 | H | A-44 | H | tBu | m-D4 | H | A-44 | H |
| 45682 | 1 | H | m-D4 | H | A-45 | H | tBu | m-D4 | H | A-45 | H |
| 45683 | 1 | H | m-D4 | H | A-46 | H | tBu | m-D4 | H | A-46 | H |
| 45684 | 1 | H | m-D4 | H | A-47 | H | tBu | m-D4 | H | A-47 | H |
| 45685 | 1 | H | m-D5 | H | A-1 | H | tBu | m-D5 | H | A-1 | H |
| 45686 | 1 | H | m-D5 | H | A-2 | H | tBu | m-D5 | H | A-2 | H |
| 45687 | 1 | H | m-D5 | H | A-3 | H | tBu | m-D5 | H | A-3 | H |
| 45688 | 1 | H | m-D5 | H | A-4 | H | tBu | m-D5 | H | A-4 | H |
| 45689 | 1 | H | m-D5 | H | A-5 | H | tBu | m-D5 | H | A-5 | H |
| 45690 | 1 | H | m-D5 | H | A-6 | H | tBu | m-D5 | H | A-6 | H |
| 45691 | 1 | H | m-D5 | H | A-7 | H | tBu | m-D5 | H | A-7 | H |
| 45692 | 1 | H | m-D5 | H | A-8 | H | tBu | m-D5 | H | A-8 | H |
| 45693 | 1 | H | m-D5 | H | A-9 | H | tBu | m-D5 | H | A-9 | H |
| 45694 | 1 | H | m-D5 | H | A-10 | H | tBu | m-D5 | H | A-10 | H |
| 45695 | 1 | H | m-D5 | H | A-11 | H | tBu | m-D5 | H | A-11 | H |
| 45696 | 1 | H | m-D5 | H | A-12 | H | tBu | m-D5 | H | A-12 | H |
| 45697 | 1 | H | m-D5 | H | A-13 | H | tBu | m-D5 | H | A-13 | H |
| 45698 | 1 | H | m-D5 | H | A-14 | H | tBu | m-D5 | H | A-14 | H |
| 45699 | 1 | H | m-D5 | H | A-15 | H | tBu | m-D5 | H | A-15 | H |
| 45700 | 1 | H | m-D5 | H | A-16 | H | tBu | m-D5 | H | A-16 | H |
| 45701 | 1 | H | m-D5 | H | A-17 | H | tBu | m-D5 | H | A-17 | H |
| 45702 | 1 | H | m-D5 | H | A-18 | H | tBu | m-D5 | H | A-18 | H |
| 45703 | 1 | H | m-D5 | H | A-19 | H | tBu | m-D5 | H | A-19 | H |
| 45704 | 1 | H | m-D5 | H | A-20 | H | tBu | m-D5 | H | A-20 | H |
| 45705 | 1 | H | m-D5 | H | A-21 | H | tBu | m-D5 | H | A-21 | H |
| 45706 | 1 | H | m-D5 | H | A-22 | H | tBu | m-D5 | H | A-22 | H |
| 45707 | 1 | H | m-D5 | H | A-23 | H | tBu | m-D5 | H | A-23 | H |
| 45708 | 1 | H | m-D5 | H | A-24 | H | tBu | m-D5 | H | A-24 | H |
| 45709 | 1 | H | m-D5 | H | A-25 | H | tBu | m-D5 | H | A-25 | H |
| 45710 | 1 | H | m-D5 | H | A-26 | H | tBu | m-D5 | H | A-26 | H |
| 45711 | 1 | H | m-D5 | H | A-27 | H | tBu | m-D5 | H | A-27 | H |
| 45712 | 1 | H | m-D5 | H | A-28 | H | tBu | m-D5 | H | A-28 | H |
| 45713 | 1 | H | m-D5 | H | A-29 | H | tBu | m-D5 | H | A-29 | H |
| 45714 | 1 | H | m-D5 | H | A-30 | H | tBu | m-D5 | H | A-30 | H |
| 45715 | 1 | H | m-D5 | H | A-31 | H | tBu | m-D5 | H | A-31 | H |
| 45716 | 1 | H | m-D5 | H | A-32 | H | tBu | m-D5 | H | A-32 | H |
| 45717 | 1 | H | m-D5 | H | A-33 | H | tBu | m-D5 | H | A-33 | H |
| 45718 | 1 | H | m-D5 | H | A-34 | H | tBu | m-D5 | H | A-34 | H |
| 45719 | 1 | H | m-D5 | H | A-35 | H | tBu | m-D5 | H | A-35 | H |
| 45720 | 1 | H | m-D5 | H | A-36 | H | tBu | m-D5 | H | A-36 | H |
| 45721 | 1 | H | m-D5 | H | A-37 | H | tBu | m-D5 | H | A-37 | H |
| 45722 | 1 | H | m-D5 | H | A-38 | H | tBu | m-D5 | H | A-38 | H |
| 45723 | 1 | H | m-D5 | H | A-39 | H | tBu | m-D5 | H | A-39 | H |
| 45724 | 1 | H | m-D5 | H | A-40 | H | tBu | m-D5 | H | A-40 | H |
| 45725 | 1 | H | m-D5 | H | A-41 | H | tBu | m-D5 | H | A-41 | H |
| 45726 | 1 | H | m-D5 | H | A-42 | H | tBu | m-D5 | H | A-42 | H |
| 45727 | 1 | H | m-D5 | H | A-43 | H | tBu | m-D5 | H | A-43 | H |
| 45728 | 1 | H | m-D5 | H | A-44 | H | tBu | m-D5 | H | A-44 | H |
| 45729 | 1 | H | m-D5 | H | A-45 | H | tBu | m-D5 | H | A-45 | H |
| 45730 | 1 | H | m-D5 | H | A-46 | H | tBu | m-D5 | H | A-46 | H |
| 45731 | 1 | H | m-D5 | H | A-47 | H | tBu | m-D5 | H | A-47 | H |
| 45732 | 1 | H | m-D6 | H | A-1 | H | tBu | m-D6 | H | A-1 | H |
| 45733 | 1 | H | m-D6 | H | A-2 | H | tBu | m-D6 | H | A-2 | H |
| 45734 | 1 | H | m-D6 | H | A-3 | H | tBu | m-D6 | H | A-3 | H |
| 45735 | 1 | H | m-D6 | H | A-4 | H | tBu | m-D6 | H | A-4 | H |
| 45736 | 1 | H | m-D6 | H | A-5 | H | tBu | m-D6 | H | A-5 | H |
| 45737 | 1 | H | m-D6 | H | A-6 | H | tBu | m-D6 | H | A-6 | H |
| 45738 | 1 | H | m-D6 | H | A-7 | H | tBu | m-D6 | H | A-7 | H |
| 45739 | 1 | H | m-D6 | H | A-8 | H | tBu | m-D6 | H | A-8 | H |
| 45740 | 1 | H | m-D6 | H | A-9 | H | tBu | m-D6 | H | A-9 | H |
| 45741 | 1 | H | m-D6 | H | A-10 | H | tBu | m-D6 | H | A-10 | H |
| 45742 | 1 | H | m-D6 | H | A-11 | H | tBu | m-D6 | H | A-11 | H |
| 45743 | 1 | H | m-D6 | H | A-12 | H | tBu | m-D6 | H | A-12 | H |
| 45744 | 1 | H | m-D6 | H | A-13 | H | tBu | m-D6 | H | A-13 | H |
| 45745 | 1 | H | m-D6 | H | A-14 | H | tBu | m-D6 | H | A-14 | H |
| 45746 | 1 | H | m-D6 | H | A-15 | H | tBu | m-D6 | H | A-15 | H |
| 45747 | 1 | H | m-D6 | H | A-16 | H | tBu | m-D6 | H | A-16 | H |
| 45748 | 1 | H | m-D6 | H | A-17 | H | tBu | m-D6 | H | A-17 | H |
| 45749 | 1 | H | m-D6 | H | A-18 | H | tBu | m-D6 | H | A-18 | H |
| 45750 | 1 | H | m-D6 | H | A-19 | H | tBu | m-D6 | H | A-19 | H |
| 45751 | 1 | H | m-D6 | H | A-20 | H | tBu | m-D6 | H | A-20 | H |
| 45752 | 1 | H | m-D6 | H | A-21 | H | tBu | m-D6 | H | A-21 | H |
| 45753 | 1 | H | m-D6 | H | A-22 | H | tBu | m-D6 | H | A-22 | H |
| 45754 | 1 | H | m-D6 | H | A-23 | H | tBu | m-D6 | H | A-23 | H |
| 45755 | 1 | H | m-D6 | H | A-24 | H | tBu | m-D6 | H | A-24 | H |
| 45756 | 1 | H | m-D6 | H | A-25 | H | tBu | m-D6 | H | A-25 | H |
| 45757 | 1 | H | m-D6 | H | A-26 | H | tBu | m-D6 | H | A-26 | H |
| 45758 | 1 | H | m-D6 | H | A-27 | H | tBu | m-D6 | H | A-27 | H |
| 45759 | 1 | H | m-D6 | H | A-28 | H | tBu | m-D6 | H | A-28 | H |
| 45760 | 1 | H | m-D6 | H | A-29 | H | tBu | m-D6 | H | A-29 | H |
| 45761 | 1 | H | m-D6 | H | A-30 | H | tBu | m-D6 | H | A-30 | H |
| 45762 | 1 | H | m-D6 | H | A-31 | H | tBu | m-D6 | H | A-31 | H |
| 45763 | 1 | H | m-D6 | H | A-32 | H | tBu | m-D6 | H | A-32 | H |
| 45764 | 1 | H | m-D6 | H | A-33 | H | tBu | m-D6 | H | A-33 | H |
| 45765 | 1 | H | m-D6 | H | A-34 | H | tBu | m-D6 | H | A-34 | H |
| 45766 | 1 | H | m-D6 | H | A-35 | H | tBu | m-D6 | H | A-35 | H |
| 45767 | 1 | H | m-D6 | H | A-36 | H | tBu | m-D6 | H | A-36 | H |
| 45768 | 1 | H | m-D6 | H | A-37 | H | tBu | m-D6 | H | A-37 | H |
| 45769 | 1 | H | m-D6 | H | A-38 | H | tBu | m-D6 | H | A-38 | H |
| 45770 | 1 | H | m-D6 | H | A-39 | H | tBu | m-D6 | H | A-39 | H |
| 45771 | 1 | H | m-D6 | H | A-40 | H | tBu | m-D6 | H | A-40 | H |
| 45772 | 1 | H | m-D6 | H | A-41 | H | tBu | m-D6 | H | A-41 | H |
| 45773 | 1 | H | m-D6 | H | A-42 | H | tBu | m-D6 | H | A-42 | H |
| 45774 | 1 | H | m-D6 | H | A-43 | H | tBu | m-D6 | H | A-43 | H |
| 45775 | 1 | H | m-D6 | H | A-44 | H | tBu | m-D6 | H | A-44 | H |
| 45776 | 1 | H | m-D6 | H | A-45 | H | tBu | m-D6 | H | A-45 | H |
| 45777 | 1 | H | m-D6 | H | A-46 | H | tBu | m-D6 | H | A-46 | H |
| 45778 | 1 | H | m-D6 | H | A-47 | H | tBu | m-D6 | H | A-47 | H |
| 45779 | 1 | H | m-D7 | H | A-1 | H | tBu | m-D7 | H | A-1 | H |
| 45780 | 1 | H | m-D7 | H | A-2 | H | tBu | m-D7 | H | A-2 | H |
| 45781 | 1 | H | m-D7 | H | A-3 | H | tBu | m-D7 | H | A-3 | H |
| 45782 | 1 | H | m-D7 | H | A-4 | H | tBu | m-D7 | H | A-4 | H |
| 45783 | 1 | H | m-D7 | H | A-5 | H | tBu | m-D7 | H | A-5 | H |
| 45784 | 1 | H | m-D7 | H | A-6 | H | tBu | m-D7 | H | A-6 | H |
| 45785 | 1 | H | m-D7 | H | A-7 | H | tBu | m-D7 | H | A-7 | H |
| 45786 | 1 | H | m-D7 | H | A-8 | H | tBu | m-D7 | H | A-8 | H |
| 45787 | 1 | H | m-D7 | H | A-9 | H | tBu | m-D7 | H | A-9 | H |
| 45788 | 1 | H | m-D7 | H | A-10 | H | tBu | m-D7 | H | A-10 | H |
| 45789 | 1 | H | m-D7 | H | A-11 | H | tBu | m-D7 | H | A-11 | H |
| 45790 | 1 | H | m-D7 | H | A-12 | H | tBu | m-D7 | H | A-12 | H |
| 45791 | 1 | H | m-D7 | H | A-13 | H | tBu | m-D7 | H | A-13 | H |
| 45792 | 1 | H | m-D7 | H | A-14 | H | tBu | m-D7 | H | A-14 | H |
| 45793 | 1 | H | m-D7 | H | A-15 | H | tBu | m-D7 | H | A-15 | H |
| 45794 | 1 | H | m-D7 | H | A-16 | H | tBu | m-D7 | H | A-16 | H |
| 45795 | 1 | H | m-D7 | H | A-17 | H | tBu | m-D7 | H | A-17 | H |
| 45796 | 1 | H | m-D7 | H | A-18 | H | tBu | m-D7 | H | A-18 | H |
| 45797 | 1 | H | m-D7 | H | A-19 | H | tBu | m-D7 | H | A-19 | H |
| 45798 | 1 | H | m-D/ | H | A-20 | H | tBu | m-D7 | H | A-20 | H |
| 45799 | 1 | H | m-D7 | H | A-21 | H | tBu | m-D7 | H | A-21 | H |
| 45800 | 1 | H | m-D7 | H | A-22 | H | tBu | m-D7 | H | A-22 | H |
| 45801 | 1 | H | m-D7 | H | A-23 | H | tBu | m-D7 | H | A-23 | H |
| 45802 | 1 | H | m-D7 | H | A-24 | H | tBu | m-D7 | H | A-24 | H |
| 45803 | 1 | H | m-D7 | H | A-25 | H | tBu | m-D7 | H | A-25 | H |
| 45804 | 1 | H | m-D7 | H | A-26 | H | tBu | m-D7 | H | A-26 | H |
| 45805 | 1 | H | m-D7 | H | A-27 | H | tBu | m-D7 | H | A-27 | H |
| 45806 | 1 | H | m-D7 | H | A-28 | H | tBu | m-D7 | H | A-28 | H |
| 45807 | 1 | H | m-D7 | H | A-29 | H | tBu | m-D7 | H | A-29 | H |
| 45808 | 1 | H | m-D7 | H | A-30 | H | tBu | m-D7 | H | A-30 | H |
| 45809 | 1 | H | m-D7 | H | A-31 | H | tBu | m-D7 | H | A-31 | H |
| 45810 | 1 | H | m-D7 | H | A-32 | H | tBu | m-D7 | H | A-32 | H |
| 45811 | 1 | H | m-D7 | H | A-33 | H | tBu | m-D7 | H | A-33 | H |
| 45812 | 1 | H | m-D7 | H | A-34 | H | tBu | m-D7 | H | A-34 | H |
| 45813 | 1 | H | m-D7 | H | A-35 | H | tBu | m-D7 | H | A-35 | H |
| 45814 | 1 | H | m-D7 | H | A-36 | H | tBu | m-D7 | H | A-36 | H |
| 45815 | 1 | H | m-D7 | H | A-37 | H | tBu | m-D7 | H | A-37 | H |
| 45816 | 1 | H | m-D7 | H | A-38 | H | tBu | m-D7 | H | A-38 | H |
| 45817 | 1 | H | m-D7 | H | A-39 | H | tBu | m-D7 | H | A-39 | H |
| 45818 | 1 | H | m-D7 | H | A-40 | H | tBu | m-D7 | H | A-40 | H |
| 45819 | 1 | H | m-D7 | H | A-41 | H | tBu | m-D7 | H | A-41 | H |
| 45820 | 1 | H | m-D7 | H | A-42 | H | tBu | m-D7 | H | A-42 | H |
| 45821 | 1 | H | m-D7 | H | A-43 | H | tBu | m-D7 | H | A-43 | H |
| 45822 | 1 | H | m-D7 | H | A-44 | H | tBu | m-D7 | H | A-44 | H |
| 45823 | 1 | H | m-D7 | H | A-45 | H | tBu | m-D7 | H | A-45 | H |
| 45824 | 1 | H | m-D7 | H | A-46 | H | tBu | m-D7 | H | A-46 | H |
| 45825 | 1 | H | m-D7 | H | A-47 | H | tBu | m-D7 | H | A-47 | H |
| 45826 | 1 | H | m-D8 | H | A-1 | H | tBu | m-D8 | H | A-1 | H |
| 45827 | 1 | H | m-D8 | H | A-2 | H | tBu | m-D8 | H | A-2 | H |
| 45828 | 1 | H | m-D8 | H | A-3 | H | tBu | m-D8 | H | A-3 | H |
| 45829 | 1 | H | m-D8 | H | A-4 | H | tBu | m-D8 | H | A-4 | H |
| 45830 | 1 | H | m-D8 | H | A-5 | H | tBu | m-D8 | H | A-5 | H |
| 45831 | 1 | H | m-D8 | H | A-6 | H | tBu | m-D8 | H | A-6 | H |
| 45832 | 1 | H | m-D8 | H | A-7 | H | tBu | m-D8 | H | A-7 | H |
| 45833 | 1 | H | m-D8 | H | A-8 | H | tBu | m-D8 | H | A-8 | H |
| 45834 | 1 | H | m-D8 | H | A-9 | H | tBu | m-D8 | H | A-9 | H |
| 45835 | 1 | H | m-D8 | H | A-10 | H | tBu | m-D8 | H | A-10 | H |
| 45836 | 1 | H | m-D8 | H | A-11 | H | tBu | m-D8 | H | A-11 | H |
| 45837 | 1 | H | m-D8 | H | A-12 | H | tBu | m-D8 | H | A-12 | H |
| 45838 | 1 | H | m-D8 | H | A-13 | H | tBu | m-D8 | H | A-13 | H |
| 45839 | 1 | H | m-D8 | H | A-14 | H | tBu | m-D8 | H | A-14 | H |
| 45840 | 1 | H | m-D8 | H | A-15 | H | tBu | m-D8 | H | A-15 | H |
| 45841 | 1 | H | m-D8 | H | A-16 | H | tBu | m-D8 | H | A-16 | H |
| 45842 | 1 | H | m-D8 | H | A-17 | H | tBu | m-D8 | H | A-17 | H |
| 45843 | 1 | H | m-D8 | H | A-18 | H | tBu | m-D8 | H | A-18 | H |
| 45844 | 1 | H | m-D8 | H | A-19 | H | tBu | m-D8 | H | A-19 | H |
| 45845 | 1 | H | m-D8 | H | A-20 | H | tBu | m-D8 | H | A-20 | H |
| 45846 | 1 | H | m-D8 | H | A-21 | H | tBu | m-D8 | H | A-21 | H |
| 45847 | 1 | H | m-D8 | H | A-22 | H | tBu | m-D8 | H | A-22 | H |
| 45848 | 1 | H | m-D8 | H | A-23 | H | tBu | m-D8 | H | A-23 | H |
| 45849 | 1 | H | m-D8 | H | A-24 | H | tBu | m-D8 | H | A-24 | H |
| 45850 | 1 | H | m-D8 | H | A-25 | H | tBu | m-D8 | H | A-25 | H |
| 45851 | 1 | H | m-D8 | H | A-26 | H | tBu | m-D8 | H | A-26 | H |
| 45852 | 1 | H | m-D8 | H | A-27 | H | tBu | m-D8 | H | A-27 | H |
| 45853 | 1 | H | m-D8 | H | A-28 | H | tBu | m-D8 | H | A-28 | H |
| 45854 | 1 | H | m-D8 | H | A-29 | H | tBu | m-D8 | H | A-29 | H |
| 45855 | 1 | H | m-D8 | H | A-30 | H | tBu | m-D8 | H | A-30 | H |
| 45856 | 1 | H | m-D8 | H | A-31 | H | tBu | m-D8 | H | A-31 | H |
| 45857 | 1 | H | m-D8 | H | A-32 | H | tBu | m-D8 | H | A-32 | H |
| 45858 | 1 | H | m-D8 | H | A-33 | H | tBu | m-D8 | H | A-33 | H |
| 45859 | 1 | H | m-D8 | H | A-34 | H | tBu | m-D8 | H | A-34 | H |
| 45860 | 1 | H | m-D8 | H | A-35 | H | tBu | m-D8 | H | A-35 | H |
| 45861 | 1 | H | m-D8 | H | A-36 | H | tBu | m-D8 | H | A-36 | H |
| 45862 | 1 | H | m-D8 | H | A-37 | H | tBu | m-D8 | H | A-37 | H |
| 45863 | 1 | H | m-D8 | H | A-38 | H | tBu | m-D8 | H | A-38 | H |
| 45864 | 1 | H | m-D8 | H | A-39 | H | tBu | m-D8 | H | A-39 | H |
| 45865 | 1 | H | m-D8 | H | A-40 | H | tBu | m-D8 | H | A-40 | H |
| 45866 | 1 | H | m-D8 | H | A-41 | H | tBu | m-D8 | H | A-41 | H |
| 45867 | 1 | H | m-D8 | H | A-42 | H | tBu | m-D8 | H | A-42 | H |
| 45868 | 1 | H | m-D8 | H | A-43 | H | tBu | m-D8 | H | A-43 | H |
| 45869 | 1 | H | m-D8 | H | A-44 | H | tBu | m-D8 | H | A-44 | H |
| 45870 | 1 | H | m-D8 | H | A-45 | H | tBu | m-D8 | H | A-45 | H |
| 45871 | 1 | H | m-D8 | H | A-46 | H | tBu | m-D8 | H | A-46 | H |
| 45872 | 1 | H | m-D8 | H | A-47 | H | tBu | m-D8 | H | A-47 | H |
| 45873 | 1 | H | m-D9 | H | A-1 | H | tBu | m-D9 | H | A-1 | H |
| 45874 | 1 | H | m-D9 | H | A-2 | H | tBu | m-D9 | H | A-2 | H |
| 45875 | 1 | H | m-D9 | H | A-3 | H | tBu | m-D9 | H | A-3 | H |
| 45876 | 1 | H | m-D9 | H | A-4 | H | tBu | m-D9 | H | A-4 | H |
| 45877 | 1 | H | m-D9 | H | A-5 | H | tBu | m-D9 | H | A-5 | H |
| 45878 | 1 | H | m-D9 | H | A-6 | H | tBu | m-D9 | H | A-6 | H |
| 45879 | 1 | H | m-D9 | H | A-7 | H | tBu | m-D9 | H | A-7 | H |
| 45880 | 1 | H | m-D9 | H | A-8 | H | tBu | m-D9 | H | A-8 | H |
| 45881 | 1 | H | m-D9 | H | A-9 | H | tBu | m-D9 | H | A-9 | H |
| 45882 | 1 | H | m-D9 | H | A-10 | H | tBu | m-D9 | H | A-10 | H |
| 45883 | 1 | H | m-D9 | H | A-11 | H | tBu | m-D9 | H | A-11 | H |
| 45884 | 1 | H | m-D9 | H | A-12 | H | tBu | m-D9 | H | A-12 | H |
| 45885 | 1 | H | m-D9 | H | A-13 | H | tBu | m-D9 | H | A-13 | H |
| 45886 | 1 | H | m-D9 | H | A-14 | H | tBu | m-D9 | H | A-14 | H |
| 45887 | 1 | H | m-D9 | H | A-15 | H | tBu | m-D9 | H | A-15 | H |
| 45888 | 1 | H | m-D9 | H | A-16 | H | tBu | m-D9 | H | A-16 | H |
| 45889 | 1 | H | m-D9 | H | A-17 | H | tBu | m-D9 | H | A-17 | H |
| 45890 | 1 | H | m-D9 | H | A-18 | H | tBu | m-D9 | H | A-18 | H |
| 45891 | 1 | H | m-D9 | H | A-19 | H | tBu | m-D9 | H | A-19 | H |
| 45892 | 1 | H | m-D9 | H | A-20 | H | tBu | m-D9 | H | A-20 | H |
| 45893 | 1 | H | m-D9 | H | A-21 | H | tBu | m-D9 | H | A-21 | H |
| 45894 | 1 | H | m-D9 | H | A-22 | H | tBu | m-D9 | H | A-22 | H |
| 45895 | 1 | H | m-D9 | H | A-23 | H | tBu | m-D9 | H | A-23 | H |
| 45896 | 1 | H | m-D9 | H | A-24 | H | tBu | m-D9 | H | A-24 | H |
| 45897 | 1 | H | m-D9 | H | A-25 | H | tBu | m-D9 | H | A-25 | H |
| 45898 | 1 | H | m-D9 | H | A-26 | H | tBu | m-D9 | H | A-26 | H |
| 45899 | 1 | H | m-D9 | H | A-27 | H | tBu | m-D9 | H | A-27 | H |
| 45900 | 1 | H | m-D9 | H | A-28 | H | tBu | m-D9 | H | A-28 | H |
| 45901 | 1 | H | m-D9 | H | A-29 | H | tBu | m-D9 | H | A-29 | H |
| 45902 | 1 | H | m-D9 | H | A-30 | H | tBu | m-D9 | H | A-30 | H |
| 45903 | 1 | H | m-D9 | H | A-31 | H | tBu | m-D9 | H | A-31 | H |
| 45904 | 1 | H | m-D9 | H | A-32 | H | tBu | m-D9 | H | A-32 | H |
| 45905 | 1 | H | m-D9 | H | A-33 | H | tBu | m-D9 | H | A-33 | H |
| 45906 | 1 | H | m-D9 | H | A-34 | H | tBu | m-D9 | H | A-34 | H |
| 45907 | 1 | H | m-D9 | H | A-35 | H | tBu | m-D9 | H | A-35 | H |
| 45908 | 1 | H | m-D9 | H | A-36 | H | tBu | m-D9 | H | A-36 | H |
| 45909 | 1 | H | m-D9 | H | A-37 | H | tBu | m-D9 | H | A-37 | H |
| 45910 | 1 | H | m-D9 | H | A-38 | H | tBu | m-D9 | H | A-38 | H |
| 45911 | 1 | H | m-D9 | H | A-39 | H | tBu | m-D9 | H | A-39 | H |
| 45912 | 1 | H | m-D9 | H | A-40 | H | tBu | m-D9 | H | A-40 | H |
| 45913 | 1 | H | m-D9 | H | A-41 | H | tBu | m-D9 | H | A-41 | H |
| 45914 | 1 | H | m-D9 | H | A-42 | H | tBu | m-D9 | H | A-42 | H |
| 45915 | 1 | H | m-D9 | H | A-43 | H | tBu | m-D9 | H | A-43 | H |
| 45916 | 1 | H | m-D9 | H | A-44 | H | tBu | m-D9 | H | A-44 | H |
| 45917 | 1 | H | m-D9 | H | A-45 | H | tBu | m-D9 | H | A-45 | H |
| 45918 | 1 | H | m-D9 | H | A-46 | H | tBu | m-D9 | H | A-46 | H |
| 45919 | 1 | H | m-D9 | H | A-47 | H | tBu | m-D9 | H | A-47 | H |
| 45920 | 1 | H | m-D10 | H | A-1 | H | tBu | m-D10 | H | A-1 | H |
| 45921 | 1 | H | m-D10 | H | A-2 | H | tBu | m-D10 | H | A-2 | H |
| 45922 | 1 | H | m-D10 | H | A-3 | H | tBu | m-D10 | H | A-3 | H |
| 45923 | 1 | H | m-D10 | H | A-4 | H | tBu | m-D10 | H | A-4 | H |
| 45924 | 1 | H | m-D10 | H | A-5 | H | tBu | m-D10 | H | A-5 | H |
| 45925 | 1 | H | m-D10 | H | A-6 | H | tBu | m-D10 | H | A-6 | H |
| 45926 | 1 | H | m-D10 | H | A-7 | H | tBu | m-D10 | H | A-7 | H |
| 45927 | 1 | H | m-D10 | H | A-8 | H | tBu | m-D10 | H | A-8 | H |
| 45928 | 1 | H | m-D10 | H | A-9 | H | tBu | m-D10 | H | A-9 | H |
| 45929 | 1 | H | m-D10 | H | A-10 | H | tBu | m-D10 | H | A-10 | H |
| 45930 | 1 | H | m-D10 | H | A-11 | H | tBu | m-D10 | H | A-11 | H |
| 45931 | 1 | H | m-D10 | H | A-12 | H | tBu | m-D10 | H | A-12 | H |
| 45932 | 1 | H | m-D10 | H | A-13 | H | tBu | m-D10 | H | A-13 | H |
| 45933 | 1 | H | m-D10 | H | A-14 | H | tBu | m-D10 | H | A-14 | H |
| 45934 | 1 | H | m-D10 | H | A-15 | H | tBu | m-D10 | H | A-15 | H |
| 45935 | 1 | H | m-D10 | H | A-16 | H | tBu | m-D10 | H | A-16 | H |
| 45936 | 1 | H | m-D10 | H | A-17 | H | tBu | m-D10 | H | A-17 | H |
| 45937 | 1 | H | m-D10 | H | A-18 | H | tBu | m-D10 | H | A-18 | H |
| 45938 | 1 | H | m-D10 | H | A-19 | H | tBu | m-D10 | H | A-19 | H |
| 45939 | 1 | H | m-D10 | H | A-20 | H | tBu | m-D10 | H | A-20 | H |
| 45940 | 1 | H | m-D10 | H | A-21 | H | tBu | m-D10 | H | A-21 | H |
| 45941 | 1 | H | m-D10 | H | A-22 | H | tBu | m-D10 | H | A-22 | H |
| 45942 | 1 | H | m-D10 | H | A-23 | H | tBu | m-D10 | H | A-23 | H |
| 45943 | 1 | H | m-D10 | H | A-24 | H | tBu | m-D10 | H | A-24 | H |
| 45944 | 1 | H | m-D10 | H | A-25 | H | tBu | m-D10 | H | A-25 | H |
| 45945 | 1 | H | m-D10 | H | A-26 | H | tBu | m-D10 | H | A-26 | H |
| 45946 | 1 | H | m-D10 | H | A-27 | H | tBu | m-D10 | H | A-27 | H |
| 45947 | 1 | H | m-D10 | H | A-28 | H | tBu | m-D10 | H | A-28 | H |
| 45948 | 1 | H | m-D10 | H | A-29 | H | tBu | m-D10 | H | A-29 | H |
| 45949 | 1 | H | m-D10 | H | A-30 | H | tBu | m-D10 | H | A-30 | H |
| 45950 | 1 | H | m-D10 | H | A-31 | H | tBu | m-D10 | H | A-31 | H |
| 45951 | 1 | H | m-D10 | H | A-32 | H | tBu | m-D10 | H | A-32 | H |
| 45952 | 1 | H | m-D10 | H | A-33 | H | tBu | m-D10 | H | A-33 | H |
| 45953 | 1 | H | m-D10 | H | A-34 | H | tBu | m-D10 | H | A-34 | H |
| 45954 | 1 | H | m-D10 | H | A-35 | H | tBu | m-D10 | H | A-35 | H |
| 45955 | 1 | H | m-D10 | H | A-36 | H | tBu | m-D10 | H | A-36 | H |
| 45956 | 1 | H | m-D10 | H | A-37 | H | tBu | m-D10 | H | A-37 | H |
| 45957 | 1 | H | m-D10 | H | A-38 | H | tBu | m-D10 | H | A-38 | H |
| 45958 | 1 | H | m-D10 | H | A-39 | H | tBu | m-D10 | H | A-39 | H |
| 45959 | 1 | H | m-D10 | H | A-40 | H | tBu | m-D10 | H | A-40 | H |
| 45960 | 1 | H | m-D10 | H | A-41 | H | tBu | m-D10 | H | A-41 | H |
| 45961 | 1 | H | m-D10 | H | A-42 | H | tBu | m-D10 | H | A-42 | H |
| 45962 | 1 | H | m-D10 | H | A-43 | H | tBu | m-D10 | H | A-43 | H |
| 45963 | 1 | H | m-D10 | H | A-44 | H | tBu | m-D10 | H | A-44 | H |
| 45964 | 1 | H | m-D10 | H | A-45 | H | tBu | m-D10 | H | A-45 | H |
| 45965 | 1 | H | m-D10 | H | A-46 | H | tBu | m-D10 | H | A-46 | H |
| 45966 | 1 | H | m-D10 | H | A-47 | H | tBu | m-D10 | H | A-47 | H |
| 45967 | 1 | H | m-D11 | H | A-1 | H | tBu | m-D11 | H | A-1 | H |
| 45968 | 1 | H | m-D11 | H | A-2 | H | tBu | m-D11 | H | A-2 | H |
| 45969 | 1 | H | m-D11 | H | A-3 | H | tBu | m-D11 | H | A-3 | H |
| 45970 | 1 | H | m-D11 | H | A-4 | H | tBu | m-D11 | H | A-4 | H |
| 45971 | 1 | H | m-D11 | H | A-5 | H | tBu | m-D11 | H | A-5 | H |
| 45972 | 1 | H | m-D11 | H | A-6 | H | tBu | m-D11 | H | A-6 | H |
| 45973 | 1 | H | m-D11 | H | A-7 | H | tBu | m-D11 | H | A-7 | H |
| 45974 | 1 | H | m-D11 | H | A-8 | H | tBu | m-D11 | H | A-8 | H |
| 45975 | 1 | H | m-D11 | H | A-9 | H | tBu | m-D11 | H | A-9 | H |
| 45976 | 1 | H | m-D11 | H | A-10 | H | tBu | m-D11 | H | A-10 | H |
| 45977 | 1 | H | m-D11 | H | A-11 | H | tBu | m-D11 | H | A-11 | H |
| 45978 | 1 | H | m-D11 | H | A-12 | H | tBu | m-D11 | H | A-12 | H |
| 45979 | 1 | H | m-D11 | H | A-13 | H | tBu | m-D11 | H | A-13 | H |
| 45980 | 1 | H | m-D11 | H | A-14 | H | tBu | m-D11 | H | A-14 | H |
| 45981 | 1 | H | m-D11 | H | A-15 | H | tBu | m-D11 | H | A-15 | H |
| 45982 | 1 | H | m-D11 | H | A-16 | H | tBu | m-D11 | H | A-16 | H |
| 45983 | 1 | H | m-D11 | H | A-17 | H | tBu | m-D11 | H | A-17 | H |
| 45984 | 1 | H | m-D11 | H | A-18 | H | tBu | m-D11 | H | A-18 | H |
| 45985 | 1 | H | m-D11 | H | A-19 | H | tBu | m-D11 | H | A-19 | H |
| 45986 | 1 | H | m-D11 | H | A-20 | H | tBu | m-D11 | H | A-20 | H |
| 45987 | 1 | H | m-D11 | H | A-21 | H | tBu | m-D11 | H | A-21 | H |
| 45988 | 1 | H | m-D11 | H | A-22 | H | tBu | m-D11 | H | A-22 | H |
| 45989 | 1 | H | m-D11 | H | A-23 | H | tBu | m-D11 | H | A-23 | H |
| 45990 | 1 | H | m-D11 | H | A-24 | H | tBu | m-D11 | H | A-24 | H |
| 45991 | 1 | H | m-D11 | H | A-25 | H | tBu | m-D11 | H | A-25 | H |
| 45992 | 1 | H | m-D11 | H | A-26 | H | tBu | m-D11 | H | A-26 | H |
| 45993 | 1 | H | m-D11 | H | A-27 | H | tBu | m-D11 | H | A-27 | H |
| 45994 | 1 | H | m-D11 | H | A-28 | H | tBu | m-D11 | H | A-28 | H |
| 45995 | 1 | H | m-D11 | H | A-29 | H | tBu | m-D11 | H | A-29 | H |
| 45996 | 1 | H | m-D11 | H | A-30 | H | tBu | m-D11 | H | A-30 | H |
| 45997 | 1 | H | m-D11 | H | A-31 | H | tBu | m-D11 | H | A-31 | H |
| 45998 | 1 | H | m-D11 | H | A-32 | H | tBu | m-D11 | H | A-32 | H |
| 45999 | 1 | H | m-D11 | H | A-33 | H | tBu | m-D11 | H | A-33 | H |
| 46000 | 1 | H | m-D11 | H | A-34 | H | tBu | m-D11 | H | A-34 | H |
| 46001 | 1 | H | m-D11 | H | A-35 | H | tBu | m-D11 | H | A-35 | H |
| 46002 | 1 | H | m-D11 | H | A-36 | H | tBu | m-D11 | H | A-36 | H |
| 46003 | 1 | H | m-D11 | H | A-37 | H | tBu | m-D11 | H | A-37 | H |
| 46004 | 1 | H | m-D11 | H | A-38 | H | tBu | m-D11 | H | A-38 | H |
| 46005 | 1 | H | m-D11 | H | A-39 | H | tBu | m-D11 | H | A-39 | H |
| 46006 | 1 | H | m-D11 | H | A-40 | H | tBu | m-D11 | H | A-40 | H |
| 46007 | 1 | H | m-D11 | H | A-41 | H | tBu | m-D11 | H | A-41 | H |
| 46008 | 1 | H | m-D11 | H | A-42 | H | tBu | m-D11 | H | A-42 | H |
| 46009 | 1 | H | m-D11 | H | A-43 | H | tBu | m-D11 | H | A-43 | H |
| 46010 | 1 | H | m-D11 | H | A-44 | H | tBu | m-D11 | H | A-44 | H |
| 46011 | 1 | H | m-D11 | H | A-45 | H | tBu | m-D11 | H | A-45 | H |
| 46012 | 1 | H | m-D11 | H | A-46 | H | tBu | m-D11 | H | A-46 | H |
| 46013 | 1 | H | m-D11 | H | A-47 | H | tBu | m-D11 | H | A-47 | H |
| 46014 | 1 | H | m-D12 | H | A-1 | H | tBu | m-D12 | H | A-1 | H |
| 46015 | 1 | H | m-D12 | H | A-2 | H | tBu | m-D12 | H | A-2 | H |
| 46016 | 1 | H | m-D12 | H | A-3 | H | tBu | m-D12 | H | A-3 | H |
| 46017 | 1 | H | m-D12 | H | A-4 | H | tBu | m-D12 | H | A-4 | H |
| 46018 | 1 | H | m-D12 | H | A-5 | H | tBu | m-D12 | H | A-5 | H |
| 46019 | 1 | H | m-D12 | H | A-6 | H | tBu | m-D12 | H | A-6 | H |
| 46020 | 1 | H | m-D12 | H | A-7 | H | tBu | m-D12 | H | A-7 | H |
| 46021 | 1 | H | m-D12 | H | A-8 | H | tBu | m-D12 | H | A-8 | H |
| 46022 | 1 | H | m-D12 | H | A-9 | H | tBu | m-D12 | H | A-9 | H |
| 46023 | 1 | H | m-D12 | H | A-10 | H | tBu | m-D12 | H | A-10 | H |
| 46024 | 1 | H | m-D12 | H | A-11 | H | tBu | m-D12 | H | A-11 | H |
| 46025 | 1 | H | m-D12 | H | A-12 | H | tBu | m-D12 | H | A-12 | H |
| 46026 | 1 | H | m-D12 | H | A-13 | H | tBu | m-D12 | H | A-13 | H |
| 46027 | 1 | H | m-D12 | H | A-14 | H | tBu | m-D12 | H | A-14 | H |
| 46028 | 1 | H | m-D12 | H | A-15 | H | tBu | m-D12 | H | A-15 | H |
| 46029 | 1 | H | m-D12 | H | A-16 | H | tBu | m-D12 | H | A-16 | H |
| 46030 | 1 | H | m-D12 | H | A-17 | H | tBu | m-D12 | H | A-17 | H |
| 46031 | 1 | H | m-D12 | H | A-18 | H | tBu | m-D12 | H | A-18 | H |
| 46032 | 1 | H | m-D12 | H | A-19 | H | tBu | m-D12 | H | A-19 | H |
| 46033 | 1 | H | m-D12 | H | A-20 | H | tBu | m-D12 | H | A-20 | H |
| 46034 | 1 | H | m-D12 | H | A-21 | H | tBu | m-D12 | H | A-21 | H |
| 46035 | 1 | H | m-D12 | H | A-22 | H | tBu | m-D12 | H | A-22 | H |
| 46036 | 1 | H | m-D12 | H | A-23 | H | tBu | m-D12 | H | A-23 | H |
| 46037 | 1 | H | m-D12 | H | A-24 | H | tBu | m-D12 | H | A-24 | H |
| 46038 | 1 | H | m-D12 | H | A-25 | H | tBu | m-D12 | H | A-25 | H |
| 46039 | 1 | H | m-D12 | H | A-26 | H | tBu | m-D12 | H | A-26 | H |
| 46040 | 1 | H | m-D12 | H | A-27 | H | tBu | m-D12 | H | A-27 | H |
| 46041 | 1 | H | m-D12 | H | A-28 | H | tBu | m-D12 | H | A-28 | H |
| 46042 | 1 | H | m-D12 | H | A-29 | H | tBu | m-D12 | H | A-29 | H |
| 46043 | 1 | H | m-D12 | H | A-30 | H | tBu | m-D12 | H | A-30 | H |
| 46044 | 1 | H | m-D12 | H | A-31 | H | tBu | m-D12 | H | A-31 | H |
| 46045 | 1 | H | m-D12 | H | A-32 | H | tBu | m-D12 | H | A-32 | H |
| 46046 | 1 | H | m-D12 | H | A-33 | H | tBu | m-D12 | H | A-33 | H |
| 46047 | 1 | H | m-D12 | H | A-34 | H | tBu | m-D12 | H | A-34 | H |
| 46048 | 1 | H | m-D12 | H | A-35 | H | tBu | m-D12 | H | A-35 | H |
| 46049 | 1 | H | m-D12 | H | A-36 | H | tBu | m-D12 | H | A-36 | H |
| 46050 | 1 | H | m-D12 | H | A-37 | H | tBu | m-D12 | H | A-37 | H |
| 46051 | 1 | H | m-D12 | H | A-38 | H | tBu | m-D12 | H | A-38 | H |
| 46052 | 1 | H | m-D12 | H | A-39 | H | tBu | m-D12 | H | A-39 | H |
| 46053 | 1 | H | m-D12 | H | A-40 | H | tBu | m-D12 | H | A-40 | H |
| 46054 | 1 | H | m-D12 | H | A-41 | H | tBu | m-D12 | H | A-41 | H |
| 46055 | 1 | H | m-D12 | H | A-42 | H | tBu | m-D12 | H | A-42 | H |
| 46056 | 1 | H | m-D12 | H | A-43 | H | tBu | m-D12 | H | A-43 | H |
| 46057 | 1 | H | m-D12 | H | A-44 | H | tBu | m-D12 | H | A-44 | H |
| 46058 | 1 | H | m-D12 | H | A-45 | H | tBu | m-D12 | H | A-45 | H |
| 46059 | 1 | H | m-D12 | H | A-46 | H | tBu | m-D12 | H | A-46 | H |
| 46060 | 1 | H | m-D12 | H | A-47 | H | tBu | m-D12 | H | A-47 | H |
| 46061 | 1 | H | m-D13 | H | A-1 | H | tBu | m-D13 | H | A-1 | H |
| 46062 | 1 | H | m-D13 | H | A-2 | H | tBu | m-D13 | H | A-2 | H |
| 46063 | 1 | H | m-D13 | H | A-3 | H | tBu | m-D13 | H | A-3 | H |
| 46064 | 1 | H | m-D13 | H | A-4 | H | tBu | m-D13 | H | A-4 | H |
| 46065 | 1 | H | m-D13 | H | A-5 | H | tBu | m-D13 | H | A-5 | H |
| 46066 | 1 | H | m-D13 | H | A-6 | H | tBu | m-D13 | H | A-6 | H |
| 46067 | 1 | H | m-D13 | H | A-7 | H | tBu | m-D13 | H | A-7 | H |
| 46068 | 1 | H | m-D13 | H | A-8 | H | tBu | m-D13 | H | A-8 | H |
| 46069 | 1 | H | m-D13 | H | A-9 | H | tBu | m-D13 | H | A-9 | H |
| 46070 | 1 | H | m-D13 | H | A-10 | H | tBu | m-D13 | H | A-10 | H |
| 46071 | 1 | H | m-D13 | H | A-11 | H | tBu | m-D13 | H | A-11 | H |
| 46072 | 1 | H | m-D13 | H | A-12 | H | tBu | m-D13 | H | A-12 | H |
| 46073 | 1 | H | m-D13 | H | A-13 | H | tBu | m-D13 | H | A-13 | H |
| 46074 | 1 | H | m-D13 | H | A-14 | H | tBu | m-D13 | H | A-14 | H |
| 46075 | 1 | H | m-D13 | H | A-15 | H | tBu | m-D13 | H | A-15 | H |
| 46076 | 1 | H | m-D13 | H | A-16 | H | tBu | m-D13 | H | A-16 | H |
| 46077 | 1 | H | m-D13 | H | A-17 | H | tBu | m-D13 | H | A-17 | H |
| 46078 | 1 | H | m-D13 | H | A-18 | H | tBu | m-D13 | H | A-18 | H |
| 46079 | 1 | H | m-D13 | H | A-19 | H | tBu | m-D13 | H | A-19 | H |
| 46080 | 1 | H | m-D13 | H | A-20 | H | tBu | m-D13 | H | A-20 | H |
| 46081 | 1 | H | m-D13 | H | A-21 | H | tBu | m-D13 | H | A-21 | H |
| 46082 | 1 | H | m-D13 | H | A-22 | H | tBu | m-D13 | H | A-22 | H |
| 46083 | 1 | H | m-D13 | H | A-23 | H | tBu | m-D13 | H | A-23 | H |
| 46084 | 1 | H | m-D13 | H | A-24 | H | tBu | m-D13 | H | A-24 | H |
| 46085 | 1 | H | m-D13 | H | A-25 | H | tBu | m-D13 | H | A-25 | H |
| 46086 | 1 | H | m-D13 | H | A-26 | H | tBu | m-D13 | H | A-26 | H |
| 46087 | 1 | H | m-D13 | H | A-27 | H | tBu | m-D13 | H | A-27 | H |
| 46088 | 1 | H | m-D13 | H | A-28 | H | tBu | m-D13 | H | A-28 | H |
| 46089 | 1 | H | m-D13 | H | A-29 | H | tBu | m-D13 | H | A-29 | H |
| 46090 | 1 | H | m-D13 | H | A-30 | H | tBu | m-D13 | H | A-30 | H |
| 46091 | 1 | H | m-D13 | H | A-31 | H | tBu | m-D13 | H | A-31 | H |
| 46092 | 1 | H | m-D13 | H | A-32 | H | tBu | m-D13 | H | A-32 | H |
| 46093 | 1 | H | m-D13 | H | A-33 | H | tBu | m-D13 | H | A-33 | H |
| 46094 | 1 | H | m-D13 | H | A-34 | H | tBu | m-D13 | H | A-34 | H |
| 46095 | 1 | H | m-D13 | H | A-35 | H | tBu | m-D13 | H | A-35 | H |
| 46096 | 1 | H | m-D13 | H | A-36 | H | tBu | m-D13 | H | A-36 | H |
| 46097 | 1 | H | m-D13 | H | A-37 | H | tBu | m-D13 | H | A-37 | H |
| 46098 | 1 | H | m-D13 | H | A-38 | H | tBu | m-D13 | H | A-38 | H |
| 46099 | 1 | H | m-D13 | H | A-39 | H | tBu | m-D13 | H | A-39 | H |
| 46100 | 1 | H | m-D13 | H | A-40 | H | tBu | m-D13 | H | A-40 | H |
| 46101 | 1 | H | m-D13 | H | A-41 | H | tBu | m-D13 | H | A-41 | H |
| 46102 | 1 | H | m-D13 | H | A-42 | H | tBu | m-D13 | H | A-42 | H |
| 46103 | 1 | H | m-D13 | H | A-43 | H | tBu | m-D13 | H | A-43 | H |
| 46104 | 1 | H | m-D13 | H | A-44 | H | tBu | m-D13 | H | A-44 | H |
| 46105 | 1 | H | m-D13 | H | A-45 | H | tBu | m-D13 | H | A-45 | H |
| 46106 | 1 | H | m-D13 | H | A-46 | H | tBu | m-D13 | H | A-46 | H |
| 46107 | 1 | H | m-D13 | H | A-47 | H | tBu | m-D13 | H | A-47 | H |
| 46108 | 1 | H | m-D14 | H | A-1 | H | tBu | m-D14 | H | A-1 | H |
| 46109 | 1 | H | m-D14 | H | A-2 | H | tBu | m-D14 | H | A-2 | H |
| 46110 | 1 | H | m-D14 | H | A-3 | H | tBu | m-D14 | H | A-3 | H |
| 46111 | 1 | H | m-D14 | H | A-4 | H | tBu | m-D14 | H | A-4 | H |
| 46112 | 1 | H | m-D14 | H | A-5 | H | tBu | m-D14 | H | A-5 | H |
| 46113 | 1 | H | m-D14 | H | A-6 | H | tBu | m-D14 | H | A-6 | H |
| 46114 | 1 | H | m-D14 | H | A-7 | H | tBu | m-D14 | H | A-7 | H |
| 46115 | 1 | H | m-D14 | H | A-8 | H | tBu | m-D14 | H | A-8 | H |
| 46116 | 1 | H | m-D14 | H | A-9 | H | tBu | m-D14 | H | A-9 | H |
| 46117 | 1 | H | m-D14 | H | A-10 | H | tBu | m-D14 | H | A-10 | H |
| 46118 | 1 | H | m-D14 | H | A-11 | H | tBu | m-D14 | H | A-11 | H |
| 46119 | 1 | H | m-D14 | H | A-12 | H | tBu | m-D14 | H | A-12 | H |
| 46120 | 1 | H | m-D14 | H | A-13 | H | tBu | m-D14 | H | A-13 | H |
| 46121 | 1 | H | m-D14 | H | A-14 | H | tBu | m-D14 | H | A-14 | H |
| 46122 | 1 | H | m-D14 | H | A-15 | H | tBu | m-D14 | H | A-15 | H |
| 46123 | 1 | H | m-D14 | H | A-16 | H | tBu | m-D14 | H | A-16 | H |
| 46124 | 1 | H | m-D14 | H | A-17 | H | tBu | m-D14 | H | A-17 | H |
| 46125 | 1 | H | m-D14 | H | A-18 | H | tBu | m-D14 | H | A-18 | H |
| 46126 | 1 | H | m-D14 | H | A-19 | H | tBu | m-D14 | H | A-19 | H |
| 46127 | 1 | H | m-D14 | H | A-20 | H | tBu | m-D14 | H | A-20 | H |
| 46128 | 1 | H | m-D14 | H | A-21 | H | tBu | m-D14 | H | A-21 | H |
| 46129 | 1 | H | m-D14 | H | A-22 | H | tBu | m-D14 | H | A-22 | H |
| 46130 | 1 | H | m-D14 | H | A-23 | H | tBu | m-D14 | H | A-23 | H |
| 46131 | 1 | H | m-D14 | H | A-24 | H | tBu | m-D14 | H | A-24 | H |
| 46132 | 1 | H | m-D14 | H | A-25 | H | tBu | m-D14 | H | A-25 | H |
| 46133 | 1 | H | m-D14 | H | A-26 | H | tBu | m-D14 | H | A-26 | H |
| 46134 | 1 | H | m-D14 | H | A-27 | H | tBu | m-D14 | H | A-27 | H |
| 46135 | 1 | H | m-D14 | H | A-28 | H | tBu | m-D14 | H | A-28 | H |
| 46136 | 1 | H | m-D14 | H | A-29 | H | tBu | m-D14 | H | A-29 | H |
| 46137 | 1 | H | m-D14 | H | A-30 | H | tBu | m-D14 | H | A-30 | H |
| 46138 | 1 | H | m-D14 | H | A-31 | H | tBu | m-D14 | H | A-31 | H |
| 46139 | 1 | H | m-D14 | H | A-32 | H | tBu | m-D14 | H | A-32 | H |
| 46140 | 1 | H | m-D14 | H | A-33 | H | tBu | m-D14 | H | A-33 | H |
| 46141 | 1 | H | m-D14 | H | A-34 | H | tBu | m-D14 | H | A-34 | H |
| 46142 | 1 | H | m-D14 | H | A-35 | H | tBu | m-D14 | H | A-35 | H |
| 46143 | 1 | H | m-D14 | H | A-36 | H | tBu | m-D14 | H | A-36 | H |
| 46144 | 1 | H | m-D14 | H | A-37 | H | tBu | m-D14 | H | A-37 | H |
| 46145 | 1 | H | m-D14 | H | A-38 | H | tBu | m-D14 | H | A-38 | H |
| 46146 | 1 | H | m-D14 | H | A-39 | H | tBu | m-D14 | H | A-39 | H |
| 46147 | 1 | H | m-D14 | H | A-40 | H | tBu | m-D14 | H | A-40 | H |
| 46148 | 1 | H | m-D14 | H | A-41 | H | tBu | m-D14 | H | A-41 | H |
| 46149 | 1 | H | m-D14 | H | A-42 | H | tBu | m-D14 | H | A-42 | H |
| 46150 | 1 | H | m-D14 | H | A-43 | H | tBu | m-D14 | H | A-43 | H |
| 46151 | 1 | H | m-D14 | H | A-44 | H | tBu | m-D14 | H | A-44 | H |
| 46152 | 1 | H | m-D14 | H | A-45 | H | tBu | m-D14 | H | A-45 | H |
| 46153 | 1 | H | m-D14 | H | A-46 | H | tBu | m-D14 | H | A-46 | H |
| 46154 | 1 | H | m-D14 | H | A-47 | H | tBu | m-D14 | H | A-47 | H |

Specific examples of the compounds represented by the general formula (D) include compounds represented by the following general formula (D4) in which X¹¹ to X¹⁵, and A¹¹ each represent a group shown in the following Table 14.

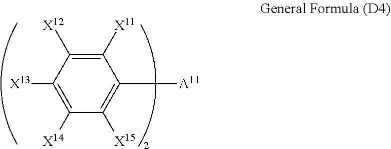

TABLE 14

| No. | x11 | x12 | x13 | x14 | x15 | A11 |
|---|---|---|---|---|---|---|
| 50001 | m-D1 | H | H | H | H | A-6 |
| 50002 | m-D1 | H | H | H | H | A-7 |
| 50003 | m-D1 | H | H | H | H | A-10 |
| 50004 | m-D1 | H | H | H | H | A-12 |
| 50005 | m-D1 | H | H | H | H | A-14 |
| 50006 | m-D1 | H | H | H | H | A-21 |
| 50007 | m-D1 | H | H | H | H | A-27 |
| 50008 | m-D1 | H | H | H | H | A-30 |
| 50009 | m-D1 | H | H | H | H | A-31 |
| 50010 | m-D1 | H | H | H | H | A-34 |
| 50011 | m-D1 | H | H | H | H | A-38 |
| 50012 | m-D1 | H | H | H | H | A-39 |
| 50013 | m-D1 | H | H | H | H | A-40 |
| 50014 | m-D1 | H | H | H | H | A-43 |
| 50015 | m-D1 | H | H | H | H | A-44 |
| 50016 | m-D1 | H | H | H | H | A-45 |
| 50017 | m-D2 | H | H | H | H | A-6 |
| 50018 | m-D2 | H | H | H | H | A-7 |
| 50019 | m-D2 | H | H | H | H | A-10 |
| 50020 | m-D2 | H | H | H | H | A-12 |
| 50021 | m-D2 | H | H | H | H | A-14 |
| 50022 | m-D2 | H | H | H | H | A-21 |
| 50023 | m-D2 | H | H | H | H | A-27 |
| 50024 | m-D2 | H | H | H | H | A-30 |
| 50025 | m-D2 | H | H | H | H | A-31 |
| 50026 | m-D2 | H | H | H | H | A-34 |
| 50027 | m-D2 | H | H | H | H | A-38 |
| 50028 | m-D2 | H | H | H | H | A-39 |
| 50029 | m-D2 | H | H | H | H | A-40 |
| 50030 | m-D2 | H | H | H | H | A-43 |
| 50031 | m-D2 | H | H | H | H | A-44 |
| 50032 | m-D2 | H | H | H | H | A-45 |
| 50033 | m-D3 | H | H | H | H | A-6 |
| 50034 | m-D3 | H | H | H | H | A-7 |
| 50035 | m-D3 | H | H | H | H | A-10 |
| 50036 | m-D3 | H | H | H | H | A-12 |
| 50037 | m-D3 | H | H | H | H | A-14 |
| 50038 | m-D3 | H | H | H | H | A-21 |
| 50039 | m-D3 | H | H | H | H | A-27 |
| 50040 | m-D3 | H | H | H | H | A-30 |
| 50041 | m-D3 | H | H | H | H | A-31 |
| 50042 | m-D3 | H | H | H | H | A-34 |
| 50043 | m-D3 | H | H | H | H | A-38 |
| 50044 | m-D3 | H | H | H | H | A-39 |
| 50045 | m-D3 | H | H | H | H | A-40 |
| 50046 | m-D3 | H | H | H | H | A-43 |
| 50047 | m-D3 | H | H | H | H | A-44 |
| 50048 | m-D3 | H | H | H | H | A-45 |
| 50049 | m-D4 | H | H | H | H | A-6 |
| 50050 | m-D4 | H | H | H | H | A-7 |
| 50051 | m-D4 | H | H | H | H | A-10 |
| 50052 | m-D4 | H | H | H | H | A-12 |
| 50053 | m-D4 | H | H | H | H | A-14 |
| 50054 | m-D4 | H | H | H | H | A-21 |
| 50055 | m-D4 | H | H | H | H | A-27 |
| 50056 | m-D4 | H | H | H | H | A-30 |
| 50057 | m-D4 | H | H | H | H | A-31 |
| 50058 | m-D4 | H | H | H | H | A-34 |
| 50059 | m-D4 | H | H | H | H | A-38 |
| 50060 | m-D4 | H | H | H | H | A-39 |
| 50061 | m-D4 | H | H | H | H | A-40 |
| 50062 | m-D4 | H | H | H | H | A-43 |
| 50063 | m-D4 | H | H | H | H | A-44 |
| 50064 | m-D4 | H | H | H | H | A-45 |
| 50065 | m-D5 | H | H | H | H | A-6 |
| 50066 | m-D5 | H | H | H | H | A-7 |
| 50067 | m-D5 | H | H | H | H | A-10 |
| 50068 | m-D5 | H | H | H | H | A-12 |
| 50069 | m-D5 | H | H | H | H | A-14 |
| 50070 | m-D5 | H | H | H | H | A-21 |
| 50071 | m-D5 | H | H | H | H | A-27 |
| 50072 | m-D5 | H | H | H | H | A-30 |
| 50073 | m-D5 | H | H | H | H | A-31 |
| 50074 | m-D5 | H | H | H | H | A-34 |
| 50075 | m-D5 | H | H | H | H | A-38 |
| 50076 | m-D5 | H | H | H | H | A-39 |
| 50077 | m-D5 | H | H | H | H | A-40 |
| 50078 | m-D5 | H | H | H | H | A-43 |
| 50079 | m-D5 | H | H | H | H | A-44 |
| 50080 | m-D5 | H | H | H | H | A-45 |
| 50081 | m-D6 | H | H | H | H | A-6 |
| 50082 | m-D6 | H | H | H | H | A-7 |
| 50083 | m-D6 | H | H | H | H | A-10 |
| 50084 | m-D6 | H | H | H | H | A-12 |
| 50085 | m-D6 | H | H | H | H | A-14 |
| 50086 | m-D6 | H | H | H | H | A-21 |
| 50087 | m-D6 | H | H | H | H | A-27 |
| 50088 | m-D6 | H | H | H | H | A-30 |
| 50089 | m-D6 | H | H | H | H | A-31 |
| 50090 | m-D6 | H | H | H | H | A-34 |
| 50091 | m-D6 | H | H | H | H | A-38 |
| 50092 | m-D6 | H | H | H | H | A-39 |
| 50093 | m-D6 | H | H | H | H | A-40 |
| 50094 | m-D6 | H | H | H | H | A-43 |
| 50095 | m-D6 | H | H | H | H | A-44 |
| 50096 | m-D6 | H | H | H | H | A-45 |
| 50097 | m-D7 | H | H | H | H | A-6 |
| 50098 | m-D7 | H | H | H | H | A-7 |
| 50099 | m-D7 | H | H | H | H | A-10 |
| 50100 | m-D7 | H | H | H | H | A-12 |
| 50101 | m-D7 | H | H | H | H | A-14 |
| 50102 | m-D7 | H | H | H | H | A-21 |
| 50103 | m-D7 | H | H | H | H | A-27 |
| 50104 | m-D7 | H | H | H | H | A-30 |
| 50105 | m-D7 | H | H | H | H | A-31 |
| 50106 | m-D7 | H | H | H | H | A-34 |
| 50107 | m-D7 | H | H | H | H | A-38 |
| 50108 | m-D7 | H | H | H | H | A-39 |
| 50109 | m-D7 | H | H | H | H | A-40 |
| 50110 | m-D7 | H | H | H | H | A-43 |
| 50111 | m-D7 | H | H | H | H | A-44 |
| 50112 | m-D7 | H | H | H | H | A-45 |
| 50113 | m-D8 | H | H | H | H | A-6 |
| 50114 | m-D8 | H | H | H | H | A-7 |
| 50115 | m-D8 | H | H | H | H | A-10 |
| 50116 | m-D8 | H | H | H | H | A-12 |
| 50117 | m-D8 | H | H | H | H | A-14 |
| 50118 | m-D8 | H | H | H | H | A-21 |
| 50119 | m-D8 | H | H | H | H | A-27 |
| 50120 | m-D8 | H | H | H | H | A-30 |
| 50121 | m-D8 | H | H | H | H | A-31 |
| 50122 | m-D8 | H | H | H | H | A-34 |
| 50123 | m-D8 | H | H | H | H | A-38 |
| 50124 | m-D8 | H | H | H | H | A-39 |
| 50125 | m-D8 | H | H | H | H | A-40 |
| 50126 | m-D8 | H | H | H | H | A-43 |
| 50127 | m-D8 | H | H | H | H | A-44 |
| 50128 | m-D8 | H | H | H | H | A-45 |
| 50129 | m-D9 | H | H | H | H | A-6 |
| 50130 | m-D9 | H | H | H | H | A-7 |
| 50131 | m-D9 | H | H | H | H | A-10 |
| 50132 | m-D9 | H | H | H | H | A-12 |
| 50133 | m-D9 | H | H | H | H | A-14 |
| 50134 | m-D9 | H | H | H | H | A-21 |
| 50135 | m-D9 | H | H | H | H | A-27 |
| 50136 | m-D9 | H | H | H | H | A-30 |
| 50137 | m-D9 | H | H | H | H | A-31 |
| 50138 | m-D9 | H | H | H | H | A-34 |
| 50139 | m-D9 | H | H | H | H | A-38 |
| 50140 | m-D9 | H | H | H | H | A-39 |
| 50141 | m-D9 | H | H | H | H | A-40 |
| 50142 | m-D9 | H | H | H | H | A-43 |
| 50143 | m-D9 | H | H | H | H | A-44 |
| 50144 | m-D9 | H | H | H | H | A-45 |
| 50145 | m-D10 | H | H | H | H | A-6 |
| 50146 | m-D10 | H | H | H | H | A-7 |
| 50147 | m-D10 | H | H | H | H | A-10 |
| 50148 | m-D10 | H | H | H | H | A-12 |
| 50149 | m-D10 | H | H | H | H | A-14 |
| 50150 | m-D10 | H | H | H | H | A-21 |
| 50151 | m-D10 | H | H | H | H | A-27 |
| 50152 | m-D10 | H | H | H | H | A-30 |
| 50153 | m-D10 | H | H | H | H | A-31 |
| 50154 | m-D10 | H | H | H | H | A-34 |
| 50155 | m-D10 | H | H | H | H | A-38 |
| 50156 | m-D10 | H | H | H | H | A-39 |
| 50157 | m-D10 | H | H | H | H | A-40 |
| 50158 | m-D10 | H | H | H | H | A-43 |
| 50159 | m-D10 | H | H | H | H | A-44 |
| 50160 | m-D10 | H | H | H | H | A-45 |
| 50161 | m-D11 | H | H | H | H | A-6 |
| 50162 | m-D11 | H | H | H | H | A-7 |
| 50163 | m-D11 | H | H | H | H | A-10 |
| 50164 | m-D11 | H | H | H | H | A-12 |
| 50165 | m-D11 | H | H | H | H | A-14 |
| 50166 | m-D11 | H | H | H | H | A-21 |
| 50167 | m-D11 | H | H | H | H | A-27 |
| 50168 | m-D11 | H | H | H | H | A-30 |
| 50169 | m-D11 | H | H | H | H | A-31 |
| 50170 | m-D11 | H | H | H | H | A-34 |
| 50171 | m-D11 | H | H | H | H | A-38 |
| 50172 | m-D11 | H | H | H | H | A-39 |
| 50173 | m-D11 | H | H | H | H | A-40 |
| 50174 | m-D11 | H | H | H | H | A-43 |
| 50175 | m-D11 | H | H | H | H | A-44 |
| 50176 | m-D11 | H | H | H | H | A-45 |
| 50177 | m-D12 | H | H | H | H | A-6 |
| 50178 | m-D12 | H | H | H | H | A-7 |
| 50179 | m-D12 | H | H | H | H | A-10 |
| 50180 | m-D12 | H | H | H | H | A-12 |
| 50181 | m-D12 | H | H | H | H | A-14 |
| 50182 | m-D12 | H | H | H | H | A-21 |
| 50183 | m-D12 | H | H | H | H | A-27 |
| 50184 | m-D12 | H | H | H | H | A-30 |
| 50185 | m-D12 | H | H | H | H | A-31 |
| 50186 | m-D12 | H | H | H | H | A-34 |
| 50187 | m-D12 | H | H | H | H | A-38 |
| 50188 | m-D12 | H | H | H | H | A-39 |
| 50189 | m-D12 | H | H | H | H | A-40 |
| 50190 | m-D12 | H | H | H | H | A-43 |
| 50191 | m-D12 | H | H | H | H | A-44 |
| 50192 | m-D12 | H | H | H | H | A-45 |
| 50193 | m-D13 | H | H | H | H | A-6 |
| 50194 | m-D13 | H | H | H | H | A-7 |
| 50195 | m-D13 | H | H | H | H | A-10 |
| 50196 | m-D13 | H | H | H | H | A-12 |
| 50197 | m-D13 | H | H | H | H | A-14 |
| 50198 | m-D13 | H | H | H | H | A-21 |
| 50199 | m-D13 | H | H | H | H | A-27 |
| 50200 | m-D13 | H | H | H | H | A-30 |
| 50201 | m-D13 | H | H | H | H | A-31 |
| 50202 | m-D13 | H | H | H | H | A-34 |
| 50203 | m-D13 | H | H | H | H | A-38 |
| 50204 | m-D13 | H | H | H | H | A-39 |
| 50205 | m-D13 | H | H | H | H | A-40 |
| 50206 | m-D13 | H | H | H | H | A-43 |
| 50207 | m-D13 | H | H | H | H | A-44 |
| 50208 | m-D13 | H | H | H | H | A-45 |
| 50209 | m-D14 | H | H | H | H | A-6 |
| 50210 | m-D14 | H | H | H | H | A-7 |
| 50211 | m-D14 | H | H | H | H | A-10 |
| 50212 | m-D14 | H | H | H | H | A-12 |
| 50213 | m-D14 | H | H | H | H | A-14 |
| 50214 | m-D14 | H | H | H | H | A-21 |
| 50215 | m-D14 | H | H | H | H | A-27 |
| 50216 | m-D14 | H | H | H | H | A-30 |
| 50217 | m-D14 | H | H | H | H | A-31 |
| 50218 | m-D14 | H | H | H | H | A-34 |
| 50219 | m-D14 | H | H | H | H | A-38 |
| 50220 | m-D14 | H | H | H | H | A-39 |
| 50221 | m-D14 | H | H | H | H | A-40 |
| 50222 | m-D14 | H | H | H | H | A-43 |
| 50223 | m-D14 | H | H | H | H | A-44 |
| 50224 | m-D14 | H | H | H | H | A-45 |
| 50225 | H | m-D1 | H | H | H | A-6 |
| 50226 | H | m-D1 | H | H | H | A-7 |
| 50227 | H | m-D1 | H | H | H | A-10 |
| 50228 | H | m-D1 | H | H | H | A-12 |
| 50229 | H | m-D1 | H | H | H | A-14 |
| 50230 | H | m-D1 | H | H | H | A-21 |
| 50231 | H | m-D1 | H | H | H | A-27 |
| 50232 | H | m-D1 | H | H | H | A-30 |
| 50233 | H | m-D1 | H | H | H | A-31 |
| 50234 | H | m-D1 | H | H | H | A-34 |
| 50235 | H | m-D1 | H | H | H | A-38 |
| 50236 | H | m-D1 | H | H | H | A-39 |
| 50237 | H | m-D1 | H | H | H | A-40 |
| 50238 | H | m-D1 | H | H | H | A-43 |
| 50239 | H | m-D1 | H | H | H | A-44 |
| 50240 | H | m-D1 | H | H | H | A-45 |
| 50241 | H | m-D2 | H | H | H | A-6 |
| 50242 | H | m-D2 | H | H | H | A-7 |
| 50243 | H | m-D2 | H | H | H | A-10 |
| 50244 | H | m-D2 | H | H | H | A-12 |
| 50245 | H | m-D2 | H | H | H | A-14 |
| 50246 | H | m-D2 | H | H | H | A-21 |
| 50247 | H | m-D2 | H | H | H | A-27 |
| 50248 | H | m-D2 | H | H | H | A-30 |
| 50249 | H | m-D2 | H | H | H | A-31 |
| 50250 | H | m-D2 | H | H | H | A-34 |
| 50251 | H | m-D2 | H | H | H | A-38 |
| 50252 | H | m-D2 | H | H | H | A-39 |
| 50253 | H | m-D2 | H | H | H | A-40 |
| 50254 | H | m-D2 | H | H | H | A-43 |
| 50255 | H | m-D2 | H | H | H | A-44 |
| 50256 | H | m-D2 | H | H | H | A-45 |
| 50257 | H | m-D3 | H | H | H | A-6 |
| 50258 | H | m-D3 | H | H | H | A-7 |
| 50259 | H | m-D3 | H | H | H | A-10 |
| 50260 | H | m-D3 | H | H | H | A-12 |
| 50261 | H | m-D3 | H | H | H | A-14 |
| 50262 | H | m-D3 | H | H | H | A-21 |
| 50263 | H | m-D3 | H | H | H | A-27 |
| 50264 | H | m-D3 | H | H | H | A-30 |
| 50265 | H | m-D3 | H | H | H | A-31 |
| 50266 | H | m-D3 | H | H | H | A-34 |
| 50267 | H | m-D3 | H | H | H | A-38 |
| 50268 | H | m-D3 | H | H | H | A-39 |
| 50269 | H | m-D3 | H | H | H | A-40 |
| 50270 | H | m-D3 | H | H | H | A-43 |
| 50271 | H | m-D3 | H | H | H | A-44 |
| 50272 | H | m-D3 | H | H | H | A-45 |
| 50273 | H | m-D4 | H | H | H | A-6 |
| 50274 | H | m-D4 | H | H | H | A-7 |
| 50275 | H | m-D4 | H | H | H | A-10 |
| 50276 | H | m-D4 | H | H | H | A-12 |
| 50277 | H | m-D4 | H | H | H | A-14 |
| 50278 | H | m-D4 | H | H | H | A-21 |
| 50279 | H | m-D4 | H | H | H | A-27 |
| 50280 | H | m-D4 | H | H | H | A-30 |
| 50281 | H | m-D4 | H | H | H | A-31 |
| 50282 | H | m-D4 | H | H | H | A-34 |
| 50283 | H | m-D4 | H | H | H | A-38 |
| 50284 | H | m-D4 | H | H | H | A-39 |
| 50285 | H | m-D4 | H | H | H | A-40 |
| 50286 | H | m-D4 | H | H | H | A-43 |
| 50287 | H | m-D4 | H | H | H | A-44 |
| 50288 | H | m-D4 | H | H | H | A-45 |
| 50289 | H | m-D5 | H | H | H | A-6 |
| 50290 | H | m-D5 | H | H | H | A-7 |
| 50291 | H | m-D5 | H | H | H | A-10 |
| 50292 | H | m-D5 | H | H | H | A-12 |
| 50293 | H | m-D5 | H | H | H | A-14 |
| 50294 | H | m-D5 | H | H | H | A-21 |
| 50295 | H | m-D5 | H | H | H | A-27 |
| 50296 | H | m-D5 | H | H | H | A-30 |
| 50297 | H | m-D5 | H | H | H | A-31 |
| 50298 | H | m-D5 | H | H | H | A-34 |
| 50299 | H | m-D5 | H | H | H | A-38 |
| 50300 | H | m-D5 | H | H | H | A-39 |
| 50301 | H | m-D5 | H | H | H | A-40 |
| 50302 | H | m-D5 | H | H | H | A-43 |
| 50303 | H | m-D5 | H | H | H | A-44 |
| 50304 | H | m-D5 | H | H | H | A-45 |
| 50305 | H | m-D6 | H | H | H | A-6 |
| 50306 | H | m-D6 | H | H | H | A-7 |
| 50307 | H | m-D6 | H | H | H | A-10 |
| 50308 | H | m-D6 | H | H | H | A-12 |
| 50309 | H | m-D6 | H | H | H | A-14 |
| 50310 | H | m-D6 | H | H | H | A-21 |
| 50311 | H | m-D6 | H | H | H | A-27 |
| 50312 | H | m-D6 | H | H | H | A-30 |
| 50313 | H | m-D6 | H | H | H | A-31 |
| 50314 | H | m-D6 | H | H | H | A-34 |
| 50315 | H | m-D6 | H | H | H | A-38 |
| 50316 | H | m-D6 | H | H | H | A-39 |
| 50317 | H | m-D6 | H | H | H | A-40 |
| 50318 | H | m-D6 | H | H | H | A-43 |
| 50319 | H | m-D6 | H | H | H | A-44 |
| 50320 | H | m-D6 | H | H | H | A-45 |
| 50321 | H | m-D7 | H | H | H | A-6 |
| 50322 | H | m-D7 | H | H | H | A-7 |
| 50323 | H | m-D7 | H | H | H | A-10 |
| 50324 | H | m-D7 | H | H | H | A-12 |
| 50325 | H | m-D7 | H | H | H | A-14 |
| 50326 | H | m-D7 | H | H | H | A-21 |
| 50327 | H | m-D7 | H | H | H | A-27 |
| 50328 | H | m-D7 | H | H | H | A-30 |
| 50329 | H | m-D7 | H | H | H | A-31 |
| 50330 | H | m-D7 | H | H | H | A-34 |
| 50331 | H | m-D7 | H | H | H | A-38 |
| 50332 | H | m-D7 | H | H | H | A-39 |
| 50333 | H | m-D7 | H | H | H | A-40 |
| 50334 | H | m-D7 | H | H | H | A-43 |
| 50335 | H | m-D7 | H | H | H | A-44 |
| 50336 | H | m-D7 | H | H | H | A-45 |
| 50337 | H | m-D8 | H | H | H | A-6 |
| 50338 | H | m-D8 | H | H | H | A-7 |
| 50339 | H | m-D8 | H | H | H | A-10 |
| 50340 | H | m-D8 | H | H | H | A-12 |
| 50341 | H | m-D8 | H | H | H | A-14 |
| 50342 | H | m-D8 | H | H | H | A-21 |
| 50343 | H | m-D8 | H | H | H | A-27 |
| 50344 | H | m-D8 | H | H | H | A-30 |
| 50345 | H | m-D8 | H | H | H | A-31 |
| 50346 | H | m-D8 | H | H | H | A-34 |
| 50347 | H | m-D8 | H | H | H | A-38 |
| 50348 | H | m-D8 | H | H | H | A-39 |
| 50349 | H | m-D8 | H | H | H | A-40 |
| 50350 | H | m-D8 | H | H | H | A-43 |
| 50351 | H | m-D8 | H | H | H | A-44 |
| 50352 | H | m-D8 | H | H | H | A-45 |
| 50353 | H | m-D9 | H | H | H | A-6 |
| 50354 | H | m-D9 | H | H | H | A-7 |
| 50355 | H | m-D9 | H | H | H | A-10 |
| 50356 | H | m-D9 | H | H | H | A-12 |
| 50357 | H | m-D9 | H | H | H | A-14 |
| 50358 | H | m-D9 | H | H | H | A-21 |
| 50359 | H | m-D9 | H | H | H | A-27 |
| 50360 | H | m-D9 | H | H | H | A-30 |
| 50361 | H | m-D9 | H | H | H | A-31 |
| 50362 | H | m-D9 | H | H | H | A-34 |
| 50363 | H | m-D9 | H | H | H | A-38 |
| 50364 | H | m-D9 | H | H | H | A-39 |
| 50365 | H | m-D9 | H | H | H | A-40 |
| 50366 | H | m-D9 | H | H | H | A-43 |
| 50367 | H | m-D9 | H | H | H | A-44 |
| 50368 | H | m-D9 | H | H | H | A-45 |
| 50369 | H | m-D10 | H | H | H | A-6 |
| 50370 | H | m-D10 | H | H | H | A-7 |
| 50371 | H | m-D10 | H | H | H | A-10 |
| 50372 | H | m-D10 | H | H | H | A-12 |
| 50373 | H | m-D10 | H | H | H | A-14 |
| 50374 | H | m-D10 | H | H | H | A-21 |
| 50375 | H | m-D10 | H | H | H | A-27 |
| 50376 | H | m-D10 | H | H | H | A-30 |
| 50377 | H | m-D10 | H | H | H | A-31 |
| 50378 | H | m-D10 | H | H | H | A-34 |
| 50379 | H | m-D10 | H | H | H | A-38 |
| 50380 | H | m-D10 | H | H | H | A-39 |
| 50381 | H | m-D10 | H | H | H | A-40 |
| 50382 | H | m-D10 | H | H | H | A-43 |
| 50383 | H | m-D10 | H | H | H | A-44 |
| 50384 | H | m-D10 | H | H | H | A-45 |
| 50385 | H | m-D11 | H | H | H | A-6 |
| 50386 | H | m-D11 | H | H | H | A-7 |
| 50387 | H | m-D11 | H | H | H | A-10 |
| 50388 | H | m-D11 | H | H | H | A-12 |
| 50389 | H | m-D11 | H | H | H | A-14 |
| 50390 | H | m-D11 | H | H | H | A-21 |
| 50391 | H | m-D11 | H | H | H | A-27 |
| 50392 | H | m-D11 | H | H | H | A-30 |
| 50393 | H | m-D11 | H | H | H | A-31 |
| 50394 | H | m-D11 | H | H | H | A-34 |
| 50395 | H | m-D11 | H | H | H | A-38 |
| 50396 | H | m-D11 | H | H | H | A-39 |
| 50397 | H | m-D11 | H | H | H | A-40 |
| 50398 | H | m-D11 | H | H | H | A-43 |
| 50399 | H | m-D11 | H | H | H | A-44 |
| 50400 | H | m-D11 | H | H | H | A-45 |
| 50401 | H | m-D12 | H | H | H | A-6 |
| 50402 | H | m-D12 | H | H | H | A-7 |
| 50403 | H | m-D12 | H | H | H | A-10 |
| 50404 | H | m-D12 | H | H | H | A-12 |
| 50405 | H | m-D12 | H | H | H | A-14 |
| 50406 | H | m-D12 | H | H | H | A-21 |
| 50407 | H | m-D12 | H | H | H | A-27 |
| 50408 | H | m-D12 | H | H | H | A-30 |
| 50409 | H | m-D12 | H | H | H | A-31 |
| 50410 | H | m-D12 | H | H | H | A-34 |
| 50411 | H | m-D12 | H | H | H | A-38 |
| 50412 | H | m-D12 | H | H | H | A-39 |
| 50413 | H | m-D12 | H | H | H | A-40 |
| 50414 | H | m-D12 | H | H | H | A-43 |
| 50415 | H | m-D12 | H | H | H | A-44 |
| 50416 | H | m-D12 | H | H | H | A-45 |
| 50417 | H | m-D13 | H | H | H | A-6 |
| 50418 | H | m-D13 | H | H | H | A-7 |
| 50419 | H | m-D13 | H | H | H | A-10 |
| 50420 | H | m-D13 | H | H | H | A-12 |
| 50421 | H | m-D13 | H | H | H | A-14 |
| 50422 | H | m-D13 | H | H | H | A-21 |
| 50423 | H | m-D13 | H | H | H | A-27 |
| 50424 | H | m-D13 | H | H | H | A-30 |
| 50425 | H | m-D13 | H | H | H | A-31 |
| 50426 | H | m-D13 | H | H | H | A-34 |
| 50427 | H | m-D13 | H | H | H | A-38 |
| 50428 | H | m-D13 | H | H | H | A-39 |
| 50429 | H | m-D13 | H | H | H | A-40 |
| 50430 | H | m-D13 | H | H | H | A-43 |
| 50431 | H | m-D13 | H | H | H | A-44 |
| 50432 | H | m-D13 | H | H | H | A-45 |
| 50433 | H | m-D14 | H | H | H | A-6 |
| 50434 | H | m-D14 | H | H | H | A-7 |
| 50435 | H | m-D14 | H | H | H | A-10 |
| 50436 | H | m-D14 | H | H | H | A-12 |
| 50437 | H | m-D14 | H | H | H | A-14 |
| 50438 | H | m-D14 | H | H | H | A-21 |
| 50439 | H | m-D14 | H | H | H | A-27 |
| 50440 | H | m-D14 | H | H | H | A-30 |
| 50441 | H | m-D14 | H | H | H | A-31 |
| 50442 | H | m-D14 | H | H | H | A-34 |
| 50443 | H | m-D14 | H | H | H | A-38 |
| 50444 | H | m-D14 | H | H | H | A-39 |
| 50445 | H | m-D14 | H | H | H | A-40 |
| 50446 | H | m-D14 | H | H | H | A-43 |
| 50447 | H | m-D14 | H | H | H | A-44 |
| 50448 | H | m-D14 | H | H | H | A-45 |
| 50449 | H | H | m-D1 | H | H | A-6 |
| 50450 | H | H | m-D1 | H | H | A-7 |
| 50451 | H | H | m-D1 | H | H | A-10 |
| 50452 | H | H | m-D1 | H | H | A-12 |
| 50453 | H | H | m-D1 | H | H | A-14 |
| 50454 | H | H | m-D1 | H | H | A-21 |
| 50455 | H | H | m-D1 | H | H | A-27 |
| 50456 | H | H | m-D1 | H | H | A-30 |
| 50457 | H | H | m-D1 | H | H | A-31 |
| 50458 | H | H | m-D1 | H | H | A-34 |
| 50459 | H | H | m-D1 | H | H | A-38 |
| 50460 | H | H | m-D1 | H | H | A-39 |
| 50461 | H | H | m-D1 | H | H | A-40 |
| 50462 | H | H | m-D1 | H | H | A-43 |
| 50463 | H | H | m-D1 | H | H | A-44 |
| 50464 | H | H | m-D1 | H | H | A-45 |
| 50465 | H | H | m-D2 | H | H | A-6 |
| 50466 | H | H | m-D2 | H | H | A-7 |
| 50467 | H | H | m-D2 | H | H | A-10 |
| 50468 | H | H | m-D2 | H | H | A-12 |
| 50469 | H | H | m-D2 | H | H | A-14 |
| 50470 | H | H | m-D2 | H | H | A-21 |
| 50471 | H | H | m-D2 | H | H | A-27 |
| 50472 | H | H | m-D2 | H | H | A-30 |
| 50473 | H | H | m-D2 | H | H | A-31 |
| 50474 | H | H | m-D2 | H | H | A-34 |
| 50475 | H | H | m-D2 | H | H | A-38 |
| 50476 | H | H | m-D2 | H | H | A-39 |
| 50477 | H | H | m-D2 | H | H | A-40 |
| 50478 | H | H | m-D2 | H | H | A-43 |
| 50479 | H | H | m-D2 | H | H | A-44 |
| 50480 | H | H | m-D2 | H | H | A-45 |
| 50481 | H | H | m-D3 | H | H | A-6 |
| 50482 | H | H | m-D3 | H | H | A-7 |
| 50483 | H | H | m-D3 | H | H | A-10 |
| 50484 | H | H | m-D3 | H | H | A-12 |
| 50485 | H | H | m-D3 | H | H | A-14 |
| 50486 | H | H | m-D3 | H | H | A-21 |
| 50487 | H | H | m-D3 | H | H | A-27 |
| 50488 | H | H | m-D3 | H | H | A-30 |
| 50489 | H | H | m-D3 | H | H | A-31 |
| 50490 | H | H | m-D3 | H | H | A-34 |
| 50491 | H | H | m-D3 | H | H | A-38 |
| 50492 | H | H | m-D3 | H | H | A-39 |
| 50493 | H | H | m-D3 | H | H | A-40 |
| 50494 | H | H | m-D3 | H | H | A-43 |
| 50495 | H | H | m-D3 | H | H | A-44 |
| 50496 | H | H | m-D3 | H | H | A-45 |
| 50497 | H | H | m-D4 | H | H | A-6 |
| 50498 | H | H | m-D4 | H | H | A-7 |
| 50499 | H | H | m-D4 | H | H | A-10 |
| 50500 | H | H | m-D4 | H | H | A-12 |
| 50501 | H | H | m-D4 | H | H | A-14 |
| 50502 | H | H | m-D4 | H | H | A-21 |
| 50503 | H | H | m-D4 | H | H | A-27 |
| 50504 | H | H | m-D4 | H | H | A-30 |
| 50505 | H | H | m-D4 | H | H | A-31 |
| 50506 | H | H | m-D4 | H | H | A-34 |
| 50507 | H | H | m-D4 | H | H | A-38 |
| 50508 | H | H | m-D4 | H | H | A-39 |
| 50509 | H | H | m-D4 | H | H | A-40 |
| 50510 | H | H | m-D4 | H | H | A-43 |
| 50511 | H | H | m-D4 | H | H | A-44 |
| 50512 | H | H | m-D4 | H | H | A-45 |
| 50513 | H | H | m-D5 | H | H | A-6 |
| 50514 | H | H | m-D5 | H | H | A-7 |
| 50515 | H | H | m-D5 | H | H | A-10 |
| 50516 | H | H | m-D5 | H | H | A-12 |
| 50517 | H | H | m-D5 | H | H | A-14 |
| 50518 | H | H | m-D5 | H | H | A-21 |
| 50519 | H | H | m-D5 | H | H | A-27 |
| 50520 | H | H | m-D5 | H | H | A-30 |
| 50521 | H | H | m-D5 | H | H | A-31 |
| 50522 | H | H | m-D5 | H | H | A-34 |
| 50523 | H | H | m-D5 | H | H | A-38 |
| 50524 | H | H | m-D5 | H | H | A-39 |
| 50525 | H | H | m-D5 | H | H | A-40 |
| 50526 | H | H | m-D5 | H | H | A-43 |
| 50527 | H | H | m-D5 | H | H | A-44 |
| 50528 | H | H | m-D5 | H | H | A-45 |
| 50529 | H | H | m-D6 | H | H | A-6 |
| 50530 | H | H | m-D6 | H | H | A-7 |
| 50531 | H | H | m-D6 | H | H | A-10 |
| 50532 | H | H | m-D6 | H | H | A-12 |
| 50533 | H | H | m-D6 | H | H | A-14 |
| 50534 | H | H | m-D6 | H | H | A-21 |
| 50535 | H | H | m-D6 | H | H | A-27 |
| 50536 | H | H | m-D6 | H | H | A-30 |
| 50537 | H | H | m-D6 | H | H | A-31 |
| 50538 | H | H | m-D6 | H | H | A-34 |
| 50539 | H | H | m-D6 | H | H | A-38 |
| 50540 | H | H | m-D6 | H | H | A-39 |
| 50541 | H | H | m-D6 | H | H | A-40 |
| 50542 | H | H | m-D6 | H | H | A-43 |
| 50543 | H | H | m-D6 | H | H | A-44 |
| 50544 | H | H | m-D6 | H | H | A-45 |
| 50545 | H | H | m-D7 | H | H | A-6 |
| 50546 | H | H | m-D7 | H | H | A-7 |
| 50547 | H | H | m-D7 | H | H | A-10 |
| 50548 | H | H | m-D7 | H | H | A-12 |
| 50549 | H | H | m-D7 | H | H | A-14 |
| 50550 | H | H | m-D7 | H | H | A-21 |
| 50551 | H | H | m-D7 | H | H | A-27 |
| 50552 | H | H | m-D7 | H | H | A-30 |
| 50553 | H | H | m-D7 | H | H | A-31 |
| 50554 | H | H | m-D7 | H | H | A-34 |
| 50555 | H | H | m-D7 | H | H | A-38 |
| 50556 | H | H | m-D7 | H | H | A-39 |
| 50557 | H | H | m-D7 | H | H | A-40 |
| 50558 | H | H | m-D7 | H | H | A-43 |
| 50559 | H | H | m-D7 | H | H | A-44 |
| 50560 | H | H | m-D7 | H | H | A-45 |
| 50561 | H | H | m-D8 | H | H | A-6 |
| 50562 | H | H | m-D8 | H | H | A-7 |
| 50563 | H | H | m-D8 | H | H | A-10 |
| 50564 | H | H | m-D8 | H | H | A-12 |
| 50565 | H | H | m-D8 | H | H | A-14 |
| 50566 | H | H | m-D8 | H | H | A-21 |
| 50567 | H | H | m-D8 | H | H | A-27 |
| 50568 | H | H | m-D8 | H | H | A-30 |
| 50569 | H | H | m-D8 | H | H | A-31 |
| 50570 | H | H | m-D8 | H | H | A-34 |
| 50571 | H | H | m-D8 | H | H | A-38 |
| 50572 | H | H | m-D8 | H | H | A-39 |
| 50573 | H | H | m-D8 | H | H | A-40 |
| 50574 | H | H | m-D8 | H | H | A-43 |
| 50575 | H | H | m-D8 | H | H | A-44 |
| 50576 | H | H | m-D8 | H | H | A-45 |
| 50577 | H | H | m-D9 | H | H | A-6 |
| 50578 | H | H | m-D9 | H | H | A-7 |
| 50579 | H | H | m-D9 | H | H | A-10 |
| 50580 | H | H | m-D9 | H | H | A-12 |
| 50581 | H | H | m-D9 | H | H | A-14 |
| 50582 | H | H | m-D9 | H | H | A-21 |
| 50583 | H | H | m-D9 | H | H | A-27 |
| 50584 | H | H | m-D9 | H | H | A-30 |
| 50585 | H | H | m-D9 | H | H | A-31 |
| 50586 | H | H | m-D9 | H | H | A-34 |
| 50587 | H | H | m-D9 | H | H | A-38 |
| 50588 | H | H | m-D9 | H | H | A-39 |
| 50589 | H | H | m-D9 | H | H | A-40 |
| 50590 | H | H | m-D9 | H | H | A-43 |
| 50591 | H | H | m-D9 | H | H | A-44 |
| 50592 | H | H | m-D9 | H | H | A-45 |
| 50593 | H | H | m-D10 | H | H | A-6 |
| 50594 | H | H | m-D10 | H | H | A-7 |
| 50595 | H | H | m-D10 | H | H | A-10 |
| 50596 | H | H | m-D10 | H | H | A-12 |
| 50597 | H | H | m-D10 | H | H | A-14 |
| 50598 | H | H | m-D10 | H | H | A-21 |
| 50599 | H | H | m-D10 | H | H | A-27 |
| 50600 | H | H | m-D10 | H | H | A-30 |
| 50601 | H | H | m-D10 | H | H | A-31 |
| 50602 | H | H | m-D10 | H | H | A-34 |
| 50603 | H | H | m-D10 | H | H | A-38 |
| 50604 | H | H | m-D10 | H | H | A-39 |
| 50605 | H | H | m-D10 | H | H | A-40 |
| 50606 | H | H | m-D10 | H | H | A-43 |
| 50607 | H | H | m-D10 | H | H | A-44 |
| 50608 | H | H | m-D10 | H | H | A-45 |
| 50609 | H | H | m-D11 | H | H | A-6 |
| 50610 | H | H | m-D11 | H | H | A-7 |
| 50611 | H | H | m-D11 | H | H | A-10 |
| 50612 | H | H | m-D11 | H | H | A-12 |
| 50613 | H | H | m-D11 | H | H | A-14 |
| 50614 | H | H | m-D11 | H | H | A-21 |
| 50615 | H | H | m-D11 | H | H | A-27 |
| 50616 | H | H | m-D11 | H | H | A-30 |
| 50617 | H | H | m-D11 | H | H | A-31 |
| 50618 | H | H | m-D11 | H | H | A-34 |
| 50619 | H | H | m-D11 | H | H | A-38 |
| 50620 | H | H | m-D11 | H | H | A-39 |
| 50621 | H | H | m-D11 | H | H | A-40 |
| 50622 | H | H | m-D11 | H | H | A-43 |
| 50623 | H | H | m-D11 | H | H | A-44 |
| 50624 | H | H | m-D11 | H | H | A-45 |
| 50625 | H | H | m-D12 | H | H | A-6 |
| 50626 | H | H | m-D12 | H | H | A-7 |
| 50627 | H | H | m-D12 | H | H | A-10 |
| 50628 | H | H | m-D12 | H | H | A-12 |
| 50629 | H | H | m-D12 | H | H | A-14 |
| 50630 | H | H | m-D12 | H | H | A-21 |
| 50631 | H | H | m-D12 | H | H | A-27 |
| 50632 | H | H | m-D12 | H | H | A-30 |
| 50633 | H | H | m-D12 | H | H | A-31 |
| 50634 | H | H | m-D12 | H | H | A-34 |
| 50635 | H | H | m-D12 | H | H | A-38 |
| 50636 | H | H | m-D12 | H | H | A-39 |
| 50637 | H | H | m-D12 | H | H | A-40 |
| 50638 | H | H | m-D12 | H | H | A-43 |
| 50639 | H | H | m-D12 | H | H | A-44 |
| 50640 | H | H | m-D12 | H | H | A-45 |
| 50641 | H | H | m-D13 | H | H | A-6 |
| 50642 | H | H | m-D13 | H | H | A-7 |
| 50643 | H | H | m-D13 | H | H | A-10 |
| 50644 | H | H | m-D13 | H | H | A-12 |
| 50645 | H | H | m-D13 | H | H | A-14 |
| 50646 | H | H | m-D13 | H | H | A-21 |
| 50647 | H | H | m-D13 | H | H | A-27 |
| 50648 | H | H | m-D13 | H | H | A-30 |
| 50649 | H | H | m-D13 | H | H | A-31 |
| 50650 | H | H | m-D13 | H | H | A-34 |
| 50651 | H | H | m-D13 | H | H | A-38 |
| 50652 | H | H | m-D13 | H | H | A-39 |
| 50653 | H | H | m-D13 | H | H | A-40 |
| 50654 | H | H | m-D13 | H | H | A-43 |
| 50655 | H | H | m-D13 | H | H | A-44 |
| 50656 | H | H | m-D13 | H | H | A-45 |
| 50657 | H | H | m-D14 | H | H | A-6 |
| 50658 | H | H | m-D14 | H | H | A-7 |
| 50659 | H | H | m-D14 | H | H | A-10 |
| 50660 | H | H | m-D14 | H | H | A-12 |
| 50661 | H | H | m-D14 | H | H | A-14 |
| 50662 | H | H | m-D14 | H | H | A-21 |
| 50663 | H | H | m-D14 | H | H | A-27 |
| 50664 | H | H | m-D14 | H | H | A-30 |
| 50665 | H | H | m-D14 | H | H | A-31 |
| 50666 | H | H | m-D14 | H | H | A-34 |
| 50667 | H | H | m-D14 | H | H | A-38 |
| 50668 | H | H | m-D14 | H | H | A-39 |
| 50669 | H | H | m-D14 | H | H | A-40 |
| 50670 | H | H | m-D14 | H | H | A-43 |
| 50671 | H | H | m-D14 | H | H | A-44 |
| 50672 | H | H | m-D14 | H | H | A-45 |
| 50673 | m-D1 | m-D1 | H | H | H | A-6 |
| 50674 | m-D1 | m-D1 | H | H | H | A-7 |
| 50675 | m-D1 | m-D1 | H | H | H | A-10 |
| 50676 | m-D1 | m-D1 | H | H | H | A-12 |
| 50677 | m-D1 | m-D1 | H | H | H | A-14 |
| 50678 | m-D1 | m-D1 | H | H | H | A-21 |
| 50679 | m-D1 | m-D1 | H | H | H | A-27 |
| 50680 | m-D1 | m-D1 | H | H | H | A-30 |
| 50681 | m-D1 | m-D1 | H | H | H | A-31 |
| 50682 | m-D1 | m-D1 | H | H | H | A-34 |
| 50683 | m-D1 | m-D1 | H | H | H | A-38 |
| 50684 | m-D1 | m-D1 | H | H | H | A-39 |
| 50685 | m-D1 | m-D1 | H | H | H | A-40 |
| 50686 | m-D1 | m-D1 | H | H | H | A-43 |
| 50687 | m-D1 | m-D1 | H | H | H | A-44 |
| 50688 | m-D1 | m-D1 | H | H | H | A-45 |
| 50689 | m-D2 | m-D2 | H | H | H | A-6 |
| 50690 | m-D2 | m-D2 | H | H | H | A-7 |
| 50691 | m-D2 | m-D2 | H | H | H | A-10 |
| 50692 | m-D2 | m-D2 | H | H | H | A-12 |
| 50693 | m-D2 | m-D2 | H | H | H | A-14 |
| 50694 | m-D2 | m-D2 | H | H | H | A-21 |
| 50695 | m-D2 | m-D2 | H | H | H | A-27 |
| 50696 | m-D2 | m-D2 | H | H | H | A-30 |
| 50697 | m-D2 | m-D2 | H | H | H | A-31 |
| 50698 | m-D2 | m-D2 | H | H | H | A-34 |
| 50699 | m-D2 | m-D2 | H | H | H | A-38 |
| 50700 | m-D2 | m-D2 | H | H | H | A-39 |
| 50701 | m-D2 | m-D2 | H | H | H | A-40 |
| 50702 | m-D2 | m-D2 | H | H | H | A-43 |
| 50703 | m-D2 | m-D2 | H | H | H | A-44 |
| 50704 | m-D2 | m-D2 | H | H | H | A-45 |
| 50705 | m-D3 | m-D3 | H | H | H | A-6 |
| 50706 | m-D3 | m-D3 | H | H | H | A-7 |
| 50707 | m-D3 | m-D3 | H | H | H | A-10 |
| 50708 | m-D3 | m-D3 | H | H | H | A-12 |
| 50709 | m-D3 | m-D3 | H | H | H | A-14 |
| 50710 | m-D3 | m-D3 | H | H | H | A-21 |
| 50711 | m-D3 | m-D3 | H | H | H | A-27 |
| 50712 | m-D3 | m-D3 | H | H | H | A-30 |
| 50713 | m-D3 | m-D3 | H | H | H | A-31 |
| 50714 | m-D3 | m-D3 | H | H | H | A-34 |
| 50715 | m-D3 | m-D3 | H | H | H | A-38 |
| 50716 | m-D3 | m-D3 | H | H | H | A-39 |
| 50717 | m-D3 | m-D3 | H | H | H | A-40 |
| 50718 | m-D3 | m-D3 | H | H | H | A-43 |
| 50719 | m-D3 | m-D3 | H | H | H | A-44 |
| 50720 | m-D3 | m-D3 | H | H | H | A-45 |
| 50721 | m-D4 | m-D4 | H | H | H | A-6 |
| 50722 | m-D4 | m-D4 | H | H | H | A-7 |
| 50723 | m-D4 | m-D4 | H | H | H | A-10 |
| 50724 | m-D4 | m-D4 | H | H | H | A-12 |
| 50725 | m-D4 | m-D4 | H | H | H | A-14 |
| 50726 | m-D4 | m-D4 | H | H | H | A-21 |
| 50727 | m-D4 | m-D4 | H | H | H | A-27 |
| 50728 | m-D4 | m-D4 | H | H | H | A-30 |
| 50729 | m-D4 | m-D4 | H | H | H | A-31 |
| 50730 | m-D4 | m-D4 | H | H | H | A-34 |
| 50731 | m-D4 | m-D4 | H | H | H | A-38 |
| 50732 | m-D4 | m-D4 | H | H | H | A-39 |
| 50733 | m-D4 | m-D4 | H | H | H | A-40 |
| 50734 | m-D4 | m-D4 | H | H | H | A-43 |
| 50735 | m-D4 | m-D4 | H | H | H | A-44 |
| 50736 | m-D4 | m-D4 | H | H | H | A-45 |
| 50737 | m-D5 | m-D5 | H | H | H | A-6 |
| 50738 | m-D5 | m-D5 | H | H | H | A-7 |
| 50739 | m-D5 | m-D5 | H | H | H | A-10 |
| 50740 | m-D5 | m-D5 | H | H | H | A-12 |
| 50741 | m-D5 | m-D5 | H | H | H | A-14 |
| 50742 | m-D5 | m-D5 | H | H | H | A-21 |
| 50743 | m-D5 | m-D5 | H | H | H | A-27 |
| 50744 | m-D5 | m-D5 | H | H | H | A-30 |
| 50745 | m-D5 | m-D5 | H | H | H | A-31 |
| 50746 | m-D5 | m-D5 | H | H | H | A-34 |
| 50747 | m-D5 | m-D5 | H | H | H | A-38 |
| 50748 | m-D5 | m-D5 | H | H | H | A-39 |
| 50749 | m-D5 | m-D5 | H | H | H | A-40 |
| 50750 | m-D5 | m-D5 | H | H | H | A-43 |
| 50751 | m-D5 | m-D5 | H | H | H | A-44 |
| 50752 | m-D5 | m-D5 | H | H | H | A-45 |
| 50753 | m-D6 | m-D6 | H | H | H | A-6 |
| 50754 | m-D6 | m-D6 | H | H | H | A-7 |
| 50755 | m-D6 | m-D6 | H | H | H | A-10 |
| 50756 | m-D6 | m-D6 | H | H | H | A-12 |
| 50757 | m-D6 | m-D6 | H | H | H | A-14 |
| 50758 | m-D6 | m-D6 | H | H | H | A-21 |
| 50759 | m-D6 | m-D6 | H | H | H | A-27 |
| 50760 | m-D6 | m-D6 | H | H | H | A-30 |
| 50761 | m-D6 | m-D6 | H | H | H | A-31 |
| 50762 | m-D6 | m-D6 | H | H | H | A-34 |
| 50763 | m-D6 | m-D6 | H | H | H | A-38 |
| 50764 | m-D6 | m-D6 | H | H | H | A-39 |
| 50765 | m-D6 | m-D6 | H | H | H | A-40 |
| 50766 | m-D6 | m-D6 | H | H | H | A-43 |
| 50767 | m-D6 | m-D6 | H | H | H | A-44 |
| 50768 | m-D6 | m-D6 | H | H | H | A-45 |
| 50769 | m-D7 | m-D7 | H | H | H | A-6 |
| 50770 | m-D7 | m-D7 | H | H | H | A-7 |
| 50771 | m-D7 | m-D7 | H | H | H | A-10 |
| 50772 | m-D7 | m-D7 | H | H | H | A-12 |
| 50773 | m-D7 | m-D7 | H | H | H | A-14 |
| 50774 | m-D7 | m-D7 | H | H | H | A-21 |
| 50775 | m-D7 | m-D7 | H | H | H | A-27 |
| 50776 | m-D7 | m-D7 | H | H | H | A-30 |
| 50777 | m-D7 | m-D7 | H | H | H | A-31 |
| 50778 | m-D7 | m-D7 | H | H | H | A-34 |
| 50779 | m-D7 | m-D7 | H | H | H | A-38 |
| 50780 | m-D7 | m-D7 | H | H | H | A-39 |
| 50781 | m-D7 | m-D7 | H | H | H | A-40 |
| 50782 | m-D7 | m-D7 | H | H | H | A-43 |
| 50783 | m-D7 | m-D7 | H | H | H | A-44 |
| 50784 | m-D7 | m-D7 | H | H | H | A-45 |
| 50785 | m-D8 | m-D8 | H | H | H | A-6 |
| 50786 | m-D8 | m-D8 | H | H | H | A-7 |
| 50787 | m-D8 | m-D8 | H | H | H | A-10 |
| 50788 | m-D8 | m-D8 | H | H | H | A-12 |
| 50789 | m-D8 | m-D8 | H | H | H | A-14 |
| 50790 | m-D8 | m-D8 | H | H | H | A-21 |
| 50791 | m-D8 | m-D8 | H | H | H | A-27 |
| 50792 | m-D8 | m-D8 | H | H | H | A-30 |
| 50793 | m-D8 | m-D8 | H | H | H | A-31 |
| 50794 | m-D8 | m-D8 | H | H | H | A-34 |
| 50795 | m-D8 | m-D8 | H | H | H | A-38 |
| 50796 | m-D8 | m-D8 | H | H | H | A-39 |
| 50797 | m-D8 | m-D8 | H | H | H | A-40 |
| 50798 | m-D8 | m-D8 | H | H | H | A-43 |
| 50799 | m-D8 | m-D8 | H | H | H | A-44 |
| 50800 | m-D8 | m-D8 | H | H | H | A-45 |
| 50801 | m-D9 | m-D9 | H | H | H | A-6 |
| 50802 | m-D9 | m-D9 | H | H | H | A-7 |
| 50803 | m-D9 | m-D9 | H | H | H | A-10 |
| 50804 | m-D9 | m-D9 | H | H | H | A-12 |
| 50805 | m-D9 | m-D9 | H | H | H | A-14 |
| 50806 | m-D9 | m-D9 | H | H | H | A-21 |
| 50807 | m-D9 | m-D9 | H | H | H | A-27 |
| 50808 | m-D9 | m-D9 | H | H | H | A-30 |
| 50809 | m-D9 | m-D9 | H | H | H | A-31 |
| 50810 | m-D9 | m-D9 | H | H | H | A-34 |
| 50811 | m-D9 | m-D9 | H | H | H | A-38 |
| 50812 | m-D9 | m-D9 | H | H | H | A-39 |
| 50813 | m-D9 | m-D9 | H | H | H | A-40 |
| 50814 | m-D9 | m-D9 | H | H | H | A-43 |
| 50815 | m-D9 | m-D9 | H | H | H | A-44 |
| 50816 | m-D9 | m-D9 | H | H | H | A-45 |
| 50817 | m-D10 | m-D10 | H | H | H | A-6 |
| 50818 | m-D10 | m-D10 | H | H | H | A-7 |
| 50819 | m-D10 | m-D10 | H | H | H | A-10 |
| 50820 | m-D10 | m-D10 | H | H | H | A-12 |
| 50821 | m-D10 | m-D10 | H | H | H | A-14 |
| 50822 | m-D10 | m-D10 | H | H | H | A-21 |
| 50823 | m-D10 | m-D10 | H | H | H | A-27 |
| 50824 | m-D10 | m-D10 | H | H | H | A-30 |
| 50825 | m-D10 | m-D10 | H | H | H | A-31 |
| 50826 | m-D10 | m-D10 | H | H | H | A-34 |
| 50827 | m-D10 | m-D10 | H | H | H | A-38 |
| 50828 | m-D10 | m-D10 | H | H | H | A-39 |
| 50829 | m-D10 | m-D10 | H | H | H | A-40 |
| 50830 | m-D10 | m-D10 | H | H | H | A-43 |
| 50831 | m-D10 | m-D10 | H | H | H | A-44 |
| 50832 | m-D10 | m-D10 | H | H | H | A-45 |
| 50833 | m-D11 | m-D11 | H | H | H | A-6 |
| 50834 | m-D11 | m-D11 | H | H | H | A-7 |
| 50835 | m-D11 | m-D11 | H | H | H | A-10 |
| 50836 | m-D11 | m-D11 | H | H | H | A-12 |
| 50837 | m-D11 | m-D11 | H | H | H | A-14 |
| 50838 | m-D11 | m-D11 | H | H | H | A-21 |
| 50839 | m-D11 | m-D11 | H | H | H | A-27 |
| 50840 | m-D11 | m-D11 | H | H | H | A-30 |
| 50841 | m-D11 | m-D11 | H | H | H | A-31 |
| 50842 | m-D11 | m-D11 | H | H | H | A-34 |
| 50843 | m-D11 | m-D11 | H | H | H | A-38 |
| 50844 | m-D11 | m-D11 | H | H | H | A-39 |
| 50845 | m-D11 | m-D11 | H | H | H | A-40 |
| 50846 | m-D11 | m-D11 | H | H | H | A-43 |
| 50847 | m-D11 | m-D11 | H | H | H | A-44 |
| 50848 | m-D11 | m-D11 | H | H | H | A-45 |
| 50849 | m-D12 | m-D12 | H | H | H | A-6 |
| 50850 | m-D12 | m-D12 | H | H | H | A-7 |
| 50851 | m-D12 | m-D12 | H | H | H | A-10 |
| 50852 | m-D12 | m-D12 | H | H | H | A-12 |
| 50853 | m-D12 | m-D12 | H | H | H | A-14 |
| 50854 | m-D12 | m-D12 | H | H | H | A-21 |
| 50855 | m-D12 | m-D12 | H | H | H | A-27 |
| 50856 | m-D12 | m-D12 | H | H | H | A-30 |
| 50857 | m-D12 | m-D12 | H | H | H | A-31 |
| 50858 | m-D12 | m-D12 | H | H | H | A-34 |
| 50859 | m-D12 | m-D12 | H | H | H | A-38 |
| 50860 | m-D12 | m-D12 | H | H | H | A-39 |
| 50861 | m-D12 | m-D12 | H | H | H | A-40 |
| 50862 | m-D12 | m-D12 | H | H | H | A-43 |
| 50863 | m-D12 | m-D12 | H | H | H | A-44 |
| 50864 | m-D12 | m-D12 | H | H | H | A-45 |
| 50865 | m-D13 | m-D13 | H | H | H | A-6 |
| 50866 | m-D13 | m-D13 | H | H | H | A-7 |
| 50867 | m-D13 | m-D13 | H | H | H | A-10 |
| 50868 | m-D13 | m-D13 | H | H | H | A-12 |
| 50869 | m-D13 | m-D13 | H | H | H | A-14 |
| 50870 | m-D13 | m-D13 | H | H | H | A-21 |
| 50871 | m-D13 | m-D13 | H | H | H | A-27 |
| 50872 | m-D13 | m-D13 | H | H | H | A-30 |
| 50873 | m-D13 | m-D13 | H | H | H | A-31 |
| 50874 | m-D13 | m-D13 | H | H | H | A-34 |
| 50875 | m-D13 | m-D13 | H | H | H | A-38 |
| 50876 | m-D13 | m-D13 | H | H | H | A-39 |
| 50877 | m-D13 | m-D13 | H | H | H | A-40 |
| 50878 | m-D13 | m-D13 | H | H | H | A-43 |
| 50879 | m-D13 | m-D13 | H | H | H | A-44 |
| 50880 | m-D13 | m-D13 | H | H | H | A-45 |
| 50881 | m-D14 | m-D14 | H | H | H | A-6 |
| 50882 | m-D14 | m-D14 | H | H | H | A-7 |
| 50883 | m-D14 | m-D14 | H | H | H | A-10 |
| 50884 | m-D14 | m-D14 | H | H | H | A-12 |
| 50885 | m-D14 | m-D14 | H | H | H | A-14 |
| 50886 | m-D14 | m-D14 | H | H | H | A-21 |
| 50887 | m-D14 | m-D14 | H | H | H | A-27 |
| 50888 | m-D14 | m-D14 | H | H | H | A-30 |
| 50889 | m-D14 | m-D14 | H | H | H | A-31 |
| 50890 | m-D14 | m-D14 | H | H | H | A-34 |
| 50891 | m-D14 | m-D14 | H | H | H | A-38 |
| 50892 | m-D14 | m-D14 | H | H | H | A-39 |
| 50893 | m-D14 | m-D14 | H | H | H | A-40 |
| 50894 | m-D14 | m-D14 | H | H | H | A-43 |
| 50895 | m-D14 | m-D14 | H | H | H | A-44 |
| 50896 | m-D14 | m-D14 | H | H | H | A-45 |
| 50897 | m-D1 | H | m-D1 | H | H | A-6 |
| 50898 | m-D1 | H | m-D1 | H | H | A-7 |
| 50899 | m-D1 | H | m-D1 | H | H | A-10 |
| 50900 | m-D1 | H | m-D1 | H | H | A-12 |
| 50901 | m-D1 | H | m-D1 | H | H | A-14 |
| 50902 | m-D1 | H | m-D1 | H | H | A-21 |
| 50903 | m-D1 | H | m-D1 | H | H | A-27 |
| 50904 | m-D1 | H | m-D1 | H | H | A-30 |
| 50905 | m-D1 | H | m-D1 | H | H | A-31 |
| 50906 | m-D1 | H | m-D1 | H | H | A-34 |
| 50907 | m-D1 | H | m-D1 | H | H | A-38 |
| 50908 | m-D1 | H | m-D1 | H | H | A-39 |
| 50909 | m-D1 | H | m-D1 | H | H | A-40 |
| 50910 | m-D1 | H | m-D1 | H | H | A-43 |
| 50911 | m-D1 | H | m-D1 | H | H | A-44 |
| 50912 | m-D1 | H | m-D1 | H | H | A-45 |
| 50913 | m-D2 | H | m-D2 | H | H | A-6 |
| 50914 | m-D2 | H | m-D2 | H | H | A-7 |
| 50915 | m-D2 | H | m-D2 | H | H | A-10 |
| 50916 | m-D2 | H | m-D2 | H | H | A-12 |
| 50917 | m-D2 | H | m-D2 | H | H | A-14 |
| 50918 | m-D2 | H | m-D2 | H | H | A-21 |
| 50919 | m-D2 | H | m-D2 | H | H | A-27 |
| 50920 | m-D2 | H | m-D2 | H | H | A-30 |
| 50921 | m-D2 | H | m-D2 | H | H | A-31 |
| 50922 | m-D2 | H | m-D2 | H | H | A-34 |
| 50923 | m-D2 | H | m-D2 | H | H | A-38 |
| 50924 | m-D2 | H | m-D2 | H | H | A-39 |
| 50925 | m-D2 | H | m-D2 | H | H | A-40 |
| 50926 | m-D2 | H | m-D2 | H | H | A-43 |
| 50927 | m-D2 | H | m-D2 | H | H | A-44 |
| 50928 | m-D2 | H | m-D2 | H | H | A-45 |
| 50929 | m-D3 | H | m-D3 | H | H | A-6 |
| 50930 | m-D3 | H | m-D3 | H | H | A-7 |
| 50931 | m-D3 | H | m-D3 | H | H | A-10 |
| 50932 | m-D3 | H | m-D3 | H | H | A-12 |
| 50933 | m-D3 | H | m-D3 | H | H | A-14 |
| 50934 | m-D3 | H | m-D3 | H | H | A-21 |
| 50935 | m-D3 | H | m-D3 | H | H | A-27 |
| 50936 | m-D3 | H | m-D3 | H | H | A-30 |
| 50937 | m-D3 | H | m-D3 | H | H | A-31 |
| 50938 | m-D3 | H | m-D3 | H | H | A-34 |
| 50939 | m-D3 | H | m-D3 | H | H | A-38 |
| 50940 | m-D3 | H | m-D3 | H | H | A-39 |
| 50941 | m-D3 | H | m-D3 | H | H | A-40 |
| 50942 | m-D3 | H | m-D3 | H | H | A-43 |
| 50943 | m-D3 | H | m-D3 | H | H | A-44 |
| 50944 | m-D3 | H | m-D3 | H | H | A-45 |
| 50945 | m-D4 | H | m-D4 | H | H | A-6 |
| 50946 | m-D4 | H | m-D4 | H | H | A-7 |
| 50947 | m-D4 | H | m-D4 | H | H | A-10 |
| 50948 | m-D4 | H | m-D4 | H | H | A-12 |
| 50949 | m-D4 | H | m-D4 | H | H | A-14 |
| 50950 | m-D4 | H | m-D4 | H | H | A-21 |
| 50951 | m-D4 | H | m-D4 | H | H | A-27 |
| 50952 | m-D4 | H | m-D4 | H | H | A-30 |
| 50953 | m-D4 | H | m-D4 | H | H | A-31 |
| 50954 | m-D4 | H | m-D4 | H | H | A-34 |
| 50955 | m-D4 | H | m-D4 | H | H | A-38 |
| 50956 | m-D4 | H | m-D4 | H | H | A-39 |
| 50957 | m-D4 | H | m-D4 | H | H | A-40 |
| 50958 | m-D4 | H | m-D4 | H | H | A-43 |
| 50959 | m-D4 | H | m-D4 | H | H | A-44 |
| 50960 | m-D4 | H | m-D4 | H | H | A-45 |
| 50961 | m-D5 | H | m-D5 | H | H | A-6 |
| 50962 | m-D5 | H | m-D5 | H | H | A-7 |
| 50963 | m-D5 | H | m-D5 | H | H | A-10 |
| 50964 | m-D5 | H | m-D5 | H | H | A-12 |
| 50965 | m-D5 | H | m-D5 | H | H | A-14 |
| 50966 | m-D5 | H | m-D5 | H | H | A-21 |
| 50967 | m-D5 | H | m-D5 | H | H | A-27 |
| 50968 | m-D5 | H | m-D5 | H | H | A-30 |
| 50969 | m-D5 | H | m-D5 | H | H | A-31 |
| 50970 | m-D5 | H | m-D5 | H | H | A-34 |
| 50971 | m-D5 | H | m-D5 | H | H | A-38 |
| 50972 | m-D5 | H | m-D5 | H | H | A-39 |
| 50973 | m-D5 | H | m-D5 | H | H | A-40 |
| 50974 | m-D5 | H | m-D5 | H | H | A-43 |
| 50975 | m-D5 | H | m-D5 | H | H | A-44 |
| 50976 | m-D5 | H | m-D5 | H | H | A-45 |
| 50977 | m-D6 | H | m-D6 | H | H | A-6 |
| 50978 | m-D6 | H | m-D6 | H | H | A-7 |
| 50979 | m-D6 | H | m-D6 | H | H | A-10 |
| 50980 | m-D6 | H | m-D6 | H | H | A-12 |
| 50981 | m-D6 | H | m-D6 | H | H | A-14 |
| 50982 | m-D6 | H | m-D6 | H | H | A-21 |
| 50983 | m-D6 | H | m-D6 | H | H | A-27 |
| 50984 | m-D6 | H | m-D6 | H | H | A-30 |
| 50985 | m-D6 | H | m-D6 | H | H | A-31 |
| 50986 | m-D6 | H | m-D6 | H | H | A-34 |
| 50987 | m-D6 | H | m-D6 | H | H | A-38 |
| 50988 | m-D6 | H | m-D6 | H | H | A-39 |
| 50989 | m-D6 | H | m-D6 | H | H | A-40 |
| 50990 | m-D6 | H | m-D6 | H | H | A-43 |
| 50991 | m-D6 | H | m-D6 | H | H | A-44 |
| 50992 | m-D6 | H | m-D6 | H | H | A-45 |
| 50993 | m-D7 | H | m-D7 | H | H | A-6 |
| 50994 | m-D7 | H | m-D7 | H | H | A-7 |
| 50995 | m-D7 | H | m-D7 | H | H | A-10 |
| 50996 | m-D7 | H | m-D7 | H | H | A-12 |
| 50997 | m-D7 | H | m-D7 | H | H | A-14 |
| 50998 | m-D7 | H | m-D7 | H | H | A-21 |
| 50999 | m-D7 | H | m-D7 | H | H | A-27 |
| 51000 | m-D7 | H | m-D7 | H | H | A-30 |
| 51001 | m-D7 | H | m-D7 | H | H | A-31 |
| 51002 | m-D7 | H | m-D7 | H | H | A-34 |
| 51003 | m-D7 | H | m-D7 | H | H | A-38 |
| 51004 | m-D7 | H | m-D7 | H | H | A-39 |
| 51005 | m-D7 | H | m-D7 | H | H | A-40 |
| 51006 | m-D7 | H | m-D7 | H | H | A-43 |
| 51007 | m-D7 | H | m-D7 | H | H | A-44 |
| 51008 | m-D7 | H | m-D7 | H | H | A-45 |
| 51009 | m-D8 | H | m-D8 | H | H | A-6 |
| 51010 | m-D8 | H | m-D8 | H | H | A-7 |
| 51011 | m-D8 | H | m-D8 | H | H | A-10 |
| 51012 | m-D8 | H | m-D8 | H | H | A-12 |
| 51013 | m-D8 | H | m-D8 | H | H | A-14 |
| 51014 | m-D8 | H | m-D8 | H | H | A-21 |
| 51015 | m-D8 | H | m-D8 | H | H | A-27 |
| 51016 | m-D8 | H | m-D8 | H | H | A-30 |
| 51017 | m-D8 | H | m-D8 | H | H | A-31 |
| 51018 | m-D8 | H | m-D8 | H | H | A-34 |
| 51019 | m-D8 | H | m-D8 | H | H | A-38 |
| 51020 | m-D8 | H | m-D8 | H | H | A-39 |
| 51021 | m-D8 | H | m-D8 | H | H | A-40 |
| 51022 | m-D8 | H | m-D8 | H | H | A-43 |
| 51023 | m-D8 | H | m-D8 | H | H | A-44 |
| 51024 | m-D8 | H | m-D8 | H | H | A-45 |
| 51025 | m-D9 | H | m-D9 | H | H | A-6 |
| 51026 | m-D9 | H | m-D9 | H | H | A-7 |
| 51027 | m-D9 | H | m-D9 | H | H | A-10 |
| 51028 | m-D9 | H | m-D9 | H | H | A-12 |
| 51029 | m-D9 | H | m-D9 | H | H | A-14 |
| 51030 | m-D9 | H | m-D9 | H | H | A-21 |
| 51031 | m-D9 | H | m-D9 | H | H | A-27 |
| 51032 | m-D9 | H | m-D9 | H | H | A-30 |
| 51033 | m-D9 | H | m-D9 | H | H | A-31 |
| 51034 | m-D9 | H | m-D9 | H | H | A-34 |
| 51035 | m-D9 | H | m-D9 | H | H | A-38 |
| 51036 | m-D9 | H | m-D9 | H | H | A-39 |
| 51037 | m-D9 | H | m-D9 | H | H | A-40 |
| 51038 | m-D9 | H | m-D9 | H | H | A-43 |
| 51039 | m-D9 | H | m-D9 | H | H | A-44 |
| 51040 | m-D9 | H | m-D9 | H | H | A-45 |
| 51041 | m-D10 | H | m-D10 | H | H | A-6 |
| 51042 | m-D10 | H | m-D10 | H | H | A-7 |
| 51043 | m-D10 | H | m-D10 | H | H | A-10 |
| 51044 | m-D10 | H | m-D10 | H | H | A-12 |
| 51045 | m-D10 | H | m-D10 | H | H | A-14 |
| 51046 | m-D10 | H | m-D10 | H | H | A-21 |
| 51047 | m-D10 | H | m-D10 | H | H | A-27 |
| 51048 | m-D10 | H | m-D10 | H | H | A-30 |
| 51049 | m-D10 | H | m-D10 | H | H | A-31 |
| 51050 | m-D10 | H | m-D10 | H | H | A-34 |
| 51051 | m-D10 | H | m-D10 | H | H | A-38 |
| 51052 | m-D10 | H | m-D10 | H | H | A-39 |
| 51053 | m-D10 | H | m-D10 | H | H | A-40 |
| 51054 | m-D10 | H | m-D10 | H | H | A-43 |
| 51055 | m-D10 | H | m-D10 | H | H | A-44 |
| 51056 | m-D10 | H | m-D10 | H | H | A-45 |
| 51057 | m-D11 | H | m-D11 | H | H | A-6 |
| 51058 | m-D11 | H | m-D11 | H | H | A-7 |
| 51059 | m-D11 | H | m-D11 | H | H | A-10 |
| 51060 | m-D11 | H | m-D11 | H | H | A-12 |
| 51061 | m-D11 | H | m-D11 | H | H | A-14 |
| 51062 | m-D11 | H | m-D11 | H | H | A-21 |
| 51063 | m-D11 | H | m-D11 | H | H | A-27 |
| 51064 | m-D11 | H | m-D11 | H | H | A-30 |
| 51065 | m-D11 | H | m-D11 | H | H | A-31 |
| 51066 | m-D11 | H | m-D11 | H | H | A-34 |
| 51067 | m-D11 | H | m-D11 | H | H | A-38 |
| 51068 | m-D11 | H | m-D11 | H | H | A-39 |
| 51069 | m-D11 | H | m-D11 | H | H | A-40 |
| 51070 | m-D11 | H | m-D11 | H | H | A-43 |
| 51071 | m-D11 | H | m-D11 | H | H | A-44 |
| 51072 | m-D11 | H | m-D11 | H | H | A-45 |
| 51073 | m-D12 | H | m-D12 | H | H | A-6 |
| 51074 | m-D12 | H | m-D12 | H | H | A-7 |
| 51075 | m-D12 | H | m-D12 | H | H | A-10 |
| 51076 | m-D12 | H | m-D12 | H | H | A-12 |
| 51077 | m-D12 | H | m-D12 | H | H | A-14 |
| 51078 | m-D12 | H | m-D12 | H | H | A-21 |
| 51079 | m-D12 | H | m-D12 | H | H | A-27 |
| 51080 | m-D12 | H | m-D12 | H | H | A-30 |
| 51081 | m-D12 | H | m-D12 | H | H | A-31 |
| 51082 | m-D12 | H | m-D12 | H | H | A-34 |
| 51083 | m-D12 | H | m-D12 | H | H | A-38 |
| 51084 | m-D12 | H | m-D12 | H | H | A-39 |
| 51085 | m-D12 | H | m-D12 | H | H | A-40 |
| 51086 | m-D12 | H | m-D12 | H | H | A-43 |
| 51087 | m-D12 | H | m-D12 | H | H | A-44 |
| 51088 | m-D12 | H | m-D12 | H | H | A-45 |
| 51089 | m-D13 | H | m-D13 | H | H | A-6 |
| 51090 | m-D13 | H | m-D13 | H | H | A-7 |
| 51091 | m-D13 | H | m-D13 | H | H | A-10 |
| 51092 | m-D13 | H | m-D13 | H | H | A-12 |
| 51093 | m-D13 | H | m-D13 | H | H | A-14 |
| 51094 | m-D13 | H | m-D13 | H | H | A-21 |
| 51095 | m-D13 | H | m-D13 | H | H | A-27 |
| 51096 | m-D13 | H | m-D13 | H | H | A-30 |
| 51097 | m-D13 | H | m-D13 | H | H | A-31 |
| 51098 | m-D13 | H | m-D13 | H | H | A-34 |
| 51099 | m-D13 | H | m-D13 | H | H | A-38 |
| 51100 | m-D13 | H | m-D13 | H | H | A-39 |
| 51101 | m-D13 | H | m-D13 | H | H | A-40 |
| 51102 | m-D13 | H | m-D13 | H | H | A-43 |
| 51103 | m-D13 | H | m-D13 | H | H | A-44 |
| 51104 | m-D13 | H | m-D13 | H | H | A-45 |
| 51105 | m-D14 | H | m-D14 | H | H | A-6 |
| 51106 | m-D14 | H | m-D14 | H | H | A-7 |
| 51107 | m-D14 | H | m-D14 | H | H | A-10 |
| 51108 | m-D14 | H | m-D14 | H | H | A-12 |
| 51109 | m-D14 | H | m-D14 | H | H | A-14 |
| 51110 | m-D14 | H | m-D14 | H | H | A-21 |
| 51111 | m-D14 | H | m-D14 | H | H | A-27 |
| 51112 | m-D14 | H | m-D14 | H | H | A-30 |
| 51113 | m-D14 | H | m-D14 | H | H | A-31 |
| 51114 | m-D14 | H | m-D14 | H | H | A-34 |
| 51115 | m-D14 | H | m-D14 | H | H | A-38 |
| 51116 | m-D14 | H | m-D14 | H | H | A-39 |
| 51117 | m-D14 | H | m-D14 | H | H | A-40 |
| 51118 | m-D14 | H | m-D14 | H | H | A-43 |
| 51119 | m-D14 | H | m-D14 | H | H | A-44 |
| 51120 | m-D14 | H | m-D14 | H | H | A-45 |
| 51121 | m-D1 | H | H | m-D1 | H | A-6 |
| 51122 | m-D1 | H | H | m-D1 | H | A-7 |
| 51123 | m-D1 | H | H | m-D1 | H | A-10 |
| 51124 | m-D1 | H | H | m-D1 | H | A-12 |
| 51125 | m-D1 | H | H | m-D1 | H | A-14 |
| 51126 | m-D1 | H | H | m-D1 | H | A-21 |
| 51127 | m-D1 | H | H | m-D1 | H | A-27 |
| 51128 | m-D1 | H | H | m-D1 | H | A-30 |
| 51129 | m-D1 | H | H | m-D1 | H | A-31 |
| 51130 | m-D1 | H | H | m-D1 | H | A-34 |
| 51131 | m-D1 | H | H | m-D1 | H | A-38 |
| 51132 | m-D1 | H | H | m-D1 | H | A-39 |
| 51133 | m-D1 | H | H | m-D1 | H | A-40 |
| 51134 | m-D1 | H | H | m-D1 | H | A-43 |
| 51135 | m-D1 | H | H | m-D1 | H | A-44 |
| 51136 | m-D1 | H | H | m-D1 | H | A-45 |
| 51137 | m-D2 | H | H | m-D2 | H | A-6 |
| 51138 | m-D2 | H | H | m-D2 | H | A-7 |
| 51139 | m-D2 | H | H | m-D2 | H | A-10 |
| 51140 | m-D2 | H | H | m-D2 | H | A-12 |
| 51141 | m-D2 | H | H | m-D2 | H | A-14 |
| 51142 | m-D2 | H | H | m-D2 | H | A-21 |
| 51143 | m-D2 | H | H | m-D2 | H | A-27 |
| 51144 | m-D2 | H | H | m-D2 | H | A-30 |
| 51145 | m-D2 | H | H | m-D2 | H | A-31 |
| 51146 | m-D2 | H | H | m-D2 | H | A-34 |
| 51147 | m-D2 | H | H | m-D2 | H | A-38 |
| 51148 | m-D2 | H | H | m-D2 | H | A-39 |
| 51149 | m-D2 | H | H | m-D2 | H | A-40 |
| 51150 | m-D2 | H | H | m-D2 | H | A-43 |
| 51151 | m-D2 | H | H | m-D2 | H | A-44 |
| 51152 | m-D2 | H | H | m-D2 | H | A-45 |
| 51153 | m-D3 | H | H | m-D3 | H | A-6 |
| 51154 | m-D3 | H | H | m-D3 | H | A-7 |
| 51155 | m-D3 | H | H | m-D3 | H | A-10 |
| 51156 | m-D3 | H | H | m-D3 | H | A-12 |
| 51157 | m-D3 | H | H | m-D3 | H | A-14 |
| 51158 | m-D3 | H | H | m-D3 | H | A-21 |
| 51159 | m-D3 | H | H | m-D3 | H | A-27 |
| 51160 | m-D3 | H | H | m-D3 | H | A-30 |
| 51161 | m-D3 | H | H | m-D3 | H | A-31 |
| 51162 | m-D3 | H | H | m-D3 | H | A-34 |
| 51163 | m-D3 | H | H | m-D3 | H | A-38 |
| 51164 | m-D3 | H | H | m-D3 | H | A-39 |
| 51165 | m-D3 | H | H | m-D3 | H | A-40 |
| 51166 | m-D3 | H | H | m-D3 | H | A-43 |
| 51167 | m-D3 | H | H | m-D3 | H | A-44 |
| 51168 | m-D3 | H | H | m-D3 | H | A-45 |
| 51169 | m-D4 | H | H | m-D4 | H | A-6 |
| 51170 | m-D4 | H | H | m-D4 | H | A-7 |
| 51171 | m-D4 | H | H | m-D4 | H | A-10 |
| 51172 | m-D4 | H | H | m-D4 | H | A-12 |
| 51173 | m-D4 | H | H | m-D4 | H | A-14 |
| 51174 | m-D4 | H | H | m-D4 | H | A-21 |
| 51175 | m-D4 | H | H | m-D4 | H | A-27 |
| 51176 | m-D4 | H | H | m-D4 | H | A-30 |
| 51177 | m-D4 | H | H | m-D4 | H | A-31 |
| 51178 | m-D4 | H | H | m-D4 | H | A-34 |
| 51179 | m-D4 | H | H | m-D4 | H | A-38 |
| 51180 | m-D4 | H | H | m-D4 | H | A-39 |
| 51181 | m-D4 | H | H | m-D4 | H | A-40 |
| 51182 | m-D4 | H | H | m-D4 | H | A-43 |
| 51183 | m-D4 | H | H | m-D4 | H | A-44 |
| 51184 | m-D4 | H | H | m-D4 | H | A-45 |
| 51185 | m-D5 | H | H | m-D5 | H | A-6 |
| 51186 | m-D5 | H | H | m-D5 | H | A-7 |
| 51187 | m-D5 | H | H | m-D5 | H | A-10 |
| 51188 | m-D5 | H | H | m-D5 | H | A-12 |
| 51189 | m-D5 | H | H | m-D5 | H | A-14 |
| 51190 | m-D5 | H | H | m-D5 | H | A-21 |
| 51191 | m-D5 | H | H | m-D5 | H | A-27 |
| 51192 | m-D5 | H | H | m-D5 | H | A-30 |
| 51193 | m-D5 | H | H | m-D5 | H | A-31 |
| 51194 | m-D5 | H | H | m-D5 | H | A-34 |
| 51195 | m-D5 | H | H | m-D5 | H | A-38 |
| 51196 | m-D5 | H | H | m-D5 | H | A-39 |
| 51197 | m-D5 | H | H | m-D5 | H | A-40 |
| 51198 | m-D5 | H | H | m-D5 | H | A-43 |
| 51199 | m-D5 | H | H | m-D5 | H | A-44 |
| 51200 | m-D5 | H | H | m-D5 | H | A-45 |
| 51201 | m-D6 | H | H | m-D6 | H | A-6 |
| 51202 | m-D6 | H | H | m-D6 | H | A-7 |
| 51203 | m-D6 | H | H | m-D6 | H | A-10 |
| 51204 | m-D6 | H | H | m-D6 | H | A-12 |
| 51205 | m-D6 | H | H | m-D6 | H | A-14 |
| 51206 | m-D6 | H | H | m-D6 | H | A-21 |
| 51207 | m-D6 | H | H | m-D6 | H | A-27 |
| 51208 | m-D6 | H | H | m-D6 | H | A-30 |
| 51209 | m-D6 | H | H | m-D6 | H | A-31 |
| 51210 | m-D6 | H | H | m-D6 | H | A-34 |
| 51211 | m-D6 | H | H | m-D6 | H | A-38 |
| 51212 | m-D6 | H | H | m-D6 | H | A-39 |
| 51213 | m-D6 | H | H | m-D6 | H | A-40 |
| 51214 | m-D6 | H | H | m-D6 | H | A-43 |
| 51215 | m-D6 | H | H | m-D6 | H | A-44 |
| 51216 | m-D6 | H | H | m-D6 | H | A-45 |
| 51217 | m-D7 | H | H | m-D7 | H | A-6 |
| 51218 | m-D7 | H | H | m-D7 | H | A-7 |
| 51219 | m-D7 | H | H | m-D7 | H | A-10 |
| 51220 | m-D7 | H | H | m-D7 | H | A-12 |
| 51221 | m-D7 | H | H | m-D7 | H | A-14 |
| 51222 | m-D7 | H | H | m-D7 | H | A-21 |
| 51223 | m-D7 | H | H | m-D7 | H | A-27 |
| 51224 | m-D7 | H | H | m-D7 | H | A-30 |
| 51225 | m-D7 | H | H | m-D7 | H | A-31 |
| 51226 | m-D7 | H | H | m-D7 | H | A-34 |
| 51227 | m-D7 | H | H | m-D7 | H | A-38 |
| 51228 | m-D7 | H | H | m-D7 | H | A-39 |
| 51229 | m-D7 | H | H | m-D7 | H | A-40 |
| 51230 | m-D7 | H | H | m-D7 | H | A-43 |
| 51231 | m-D7 | H | H | m-D7 | H | A-44 |
| 51232 | m-D7 | H | H | m-D7 | H | A-45 |
| 51233 | m-D8 | H | H | m-D8 | H | A-6 |
| 51234 | m-D8 | H | H | m-D8 | H | A-7 |
| 51235 | m-D8 | H | H | m-D8 | H | A-10 |
| 51236 | m-D8 | H | H | m-D8 | H | A-12 |
| 51237 | m-D8 | H | H | m-D8 | H | A-14 |
| 51238 | m-D8 | H | H | m-D8 | H | A-21 |
| 51239 | m-D8 | H | H | m-D8 | H | A-27 |
| 51240 | m-D8 | H | H | m-D8 | H | A-30 |
| 51241 | m-D8 | H | H | m-D8 | H | A-31 |
| 51242 | m-D8 | H | H | m-D8 | H | A-34 |
| 51243 | m-D8 | H | H | m-D8 | H | A-38 |
| 51244 | m-D8 | H | H | m-D8 | H | A-39 |
| 51245 | m-D8 | H | H | m-D8 | H | A-40 |
| 51246 | m-D8 | H | H | m-D8 | H | A-43 |
| 51247 | m-D8 | H | H | m-D8 | H | A-44 |
| 51248 | m-D8 | H | H | m-D8 | H | A-45 |
| 51249 | m-D9 | H | H | m-D9 | H | A-6 |
| 51250 | m-D9 | H | H | m-D9 | H | A-7 |
| 51251 | m-D9 | H | H | m-D9 | H | A-10 |
| 51252 | m-D9 | H | H | m-D9 | H | A-12 |
| 51253 | m-D9 | H | H | m-D9 | H | A-14 |
| 51254 | m-D9 | H | H | m-D9 | H | A-21 |
| 51255 | m-D9 | H | H | m-D9 | H | A-27 |
| 51256 | m-D9 | H | H | m-D9 | H | A-30 |
| 51257 | m-D9 | H | H | m-D9 | H | A-31 |
| 51258 | m-D9 | H | H | m-D9 | H | A-34 |
| 51259 | m-D9 | H | H | m-D9 | H | A-38 |
| 51260 | m-D9 | H | H | m-D9 | H | A-39 |
| 51261 | m-D9 | H | H | m-D9 | H | A-40 |
| 51262 | m-D9 | H | H | m-D9 | H | A-43 |
| 51263 | m-D9 | H | H | m-D9 | H | A-44 |
| 51264 | m-D9 | H | H | m-D9 | H | A-45 |
| 51265 | m-D10 | H | H | m-D10 | H | A-6 |
| 51266 | m-D10 | H | H | m-D10 | H | A-7 |
| 51267 | m-D10 | H | H | m-D10 | H | A-10 |
| 51268 | m-D10 | H | H | m-D10 | H | A-12 |
| 51269 | m-D10 | H | H | m-D10 | H | A-14 |
| 51270 | m-D10 | H | H | m-D10 | H | A-21 |
| 51271 | m-D10 | H | H | m-D10 | H | A-27 |
| 51272 | m-D10 | H | H | m-D10 | H | A-30 |
| 51273 | m-D10 | H | H | m-D10 | H | A-31 |
| 51274 | m-D10 | H | H | m-D10 | H | A-34 |
| 51275 | m-D10 | H | H | m-D10 | H | A-38 |
| 51276 | m-D10 | H | H | m-D10 | H | A-39 |
| 51277 | m-D10 | H | H | m-D10 | H | A-40 |
| 51278 | m-D10 | H | H | m-D10 | H | A-43 |
| 51279 | m-D10 | H | H | m-D10 | H | A-44 |
| 51280 | m-D10 | H | H | m-D10 | H | A-45 |
| 51281 | m-D11 | H | H | m-D11 | H | A-6 |
| 51282 | m-D11 | H | H | m-D11 | H | A-7 |
| 51283 | m-D11 | H | H | m-D11 | H | A-10 |
| 51284 | m-D11 | H | H | m-D11 | H | A-12 |
| 51285 | m-D11 | H | H | m-D11 | H | A-14 |
| 51286 | m-D11 | H | H | m-D11 | H | A-21 |
| 51287 | m-D11 | H | H | m-D11 | H | A-27 |
| 51288 | m-D11 | H | H | m-D11 | H | A-30 |
| 51289 | m-D11 | H | H | m-D11 | H | A-31 |
| 51290 | m-D11 | H | H | m-D11 | H | A-34 |
| 51291 | m-D11 | H | H | m-D11 | H | A-38 |
| 51292 | m-D11 | H | H | m-D11 | H | A-39 |
| 51293 | m-D11 | H | H | m-D11 | H | A-40 |
| 51294 | m-D11 | H | H | m-D11 | H | A-43 |
| 51295 | m-D11 | H | H | m-D11 | H | A-44 |
| 51296 | m-D11 | H | H | m-D11 | H | A-45 |
| 51297 | m-D12 | H | H | m-D12 | H | A-6 |
| 51298 | m-D12 | H | H | m-D12 | H | A-7 |
| 51299 | m-D12 | H | H | m-D12 | H | A-10 |
| 51300 | m-D12 | H | H | m-D12 | H | A-12 |
| 51301 | m-D12 | H | H | m-D12 | H | A-14 |
| 51302 | m-D12 | H | H | m-D12 | H | A-21 |
| 51303 | m-D12 | H | H | m-D12 | H | A-27 |
| 51304 | m-D12 | H | H | m-D12 | H | A-30 |
| 51305 | m-D12 | H | H | m-D12 | H | A-31 |
| 51306 | m-D12 | H | H | m-D12 | H | A-34 |
| 51307 | m-D12 | H | H | m-D12 | H | A-38 |
| 51308 | m-D12 | H | H | m-D12 | H | A-39 |
| 51309 | m-D12 | H | H | m-D12 | H | A-40 |
| 51310 | m-D12 | H | H | m-D12 | H | A-43 |
| 51311 | m-D12 | H | H | m-D12 | H | A-44 |
| 51312 | m-D12 | H | H | m-D12 | H | A-45 |
| 51313 | m-D13 | H | H | m-D13 | H | A-6 |
| 51314 | m-D13 | H | H | m-D13 | H | A-7 |
| 51315 | m-D13 | H | H | m-D13 | H | A-10 |
| 51316 | m-D13 | H | H | m-D13 | H | A-12 |
| 51317 | m-D13 | H | H | m-D13 | H | A-14 |
| 51318 | m-D13 | H | H | m-D13 | H | A-21 |
| 51319 | m-D13 | H | H | m-D13 | H | A-27 |
| 51320 | m-D13 | H | H | m-D13 | H | A-30 |
| 51321 | m-D13 | H | H | m-D13 | H | A-31 |
| 51322 | m-D13 | H | H | m-D13 | H | A-34 |
| 51323 | m-D13 | H | H | m-D13 | H | A-38 |
| 51324 | m-D13 | H | H | m-D13 | H | A-39 |
| 51325 | m-D13 | H | H | m-D13 | H | A-40 |
| 51326 | m-D13 | H | H | m-D13 | H | A-43 |
| 51327 | m-D13 | H | H | m-D13 | H | A-44 |
| 51328 | m-D13 | H | H | m-D13 | H | A-45 |
| 51329 | m-D14 | H | H | m-D14 | H | A-6 |
| 51330 | m-D14 | H | H | m-D14 | H | A-7 |
| 51331 | m-D14 | H | H | m-D14 | H | A-10 |
| 51332 | m-D14 | H | H | m-D14 | H | A-12 |
| 51333 | m-D14 | H | H | m-D14 | H | A-14 |
| 51334 | m-D14 | H | H | m-D14 | H | A-21 |
| 51335 | m-D14 | H | H | m-D14 | H | A-27 |
| 51336 | m-D14 | H | H | m-D14 | H | A-30 |
| 51337 | m-D14 | H | H | m-D14 | H | A-31 |
| 51338 | m-D14 | H | H | m-D14 | H | A-34 |
| 51339 | m-D14 | H | H | m-D14 | H | A-38 |
| 51340 | m-D14 | H | H | m-D14 | H | A-39 |
| 51341 | m-D14 | H | H | m-D14 | H | A-40 |
| 51342 | m-D14 | H | H | m-D14 | H | A-43 |
| 51343 | m-D14 | H | H | m-D14 | H | A-44 |
| 51344 | m-D14 | H | H | m-D14 | H | A-45 |
| 51345 | m-D1 | H | H | H | m-D1 | A-6 |
| 51346 | m-D1 | H | H | H | m-D1 | A-7 |
| 51347 | m-D1 | H | H | H | m-D1 | A-10 |
| 51348 | m-D1 | H | H | H | m-D1 | A-12 |
| 51349 | m-D1 | H | H | H | m-D1 | A-14 |
| 51350 | m-D1 | H | H | H | m-D1 | A-21 |
| 51351 | m-D1 | H | H | H | m-D1 | A-27 |
| 51352 | m-D1 | H | H | H | m-D1 | A-30 |
| 51353 | m-D1 | H | H | H | m-D1 | A-31 |
| 51354 | m-D1 | H | H | H | m-D1 | A-34 |
| 51355 | m-D1 | H | H | H | m-D1 | A-38 |
| 51356 | m-D1 | H | H | H | m-D1 | A-39 |
| 51357 | m-D1 | H | H | H | m-D1 | A-40 |
| 51358 | m-D1 | H | H | H | m-D1 | A-43 |
| 51359 | m-D1 | H | H | H | m-D1 | A-44 |
| 51360 | m-D1 | H | H | H | m-D1 | A-45 |
| 51361 | m-D2 | H | H | H | m-D2 | A-6 |
| 51362 | m-D2 | H | H | H | m-D2 | A-7 |
| 51363 | m-D2 | H | H | H | m-D2 | A-10 |
| 51364 | m-D2 | H | H | H | m-D2 | A-12 |
| 51365 | m-D2 | H | H | H | m-D2 | A-14 |
| 51366 | m-D2 | H | H | H | m-D2 | A-21 |
| 51367 | m-D2 | H | H | H | m-D2 | A-27 |
| 51368 | m-D2 | H | H | H | m-D2 | A-30 |
| 51369 | m-D2 | H | H | H | m-D2 | A-31 |
| 51370 | m-D2 | H | H | H | m-D2 | A-34 |
| 51371 | m-D2 | H | H | H | m-D2 | A-38 |
| 51372 | m-D2 | H | H | H | m-D2 | A-39 |
| 51373 | m-D2 | H | H | H | m-D2 | A-40 |
| 51374 | m-D2 | H | H | H | m-D2 | A-43 |
| 51375 | m-D2 | H | H | H | m-D2 | A-44 |
| 51376 | m-D2 | H | H | H | m-D2 | A-45 |
| 51377 | m-D3 | H | H | H | m-D3 | A-6 |
| 51378 | m-D3 | H | H | H | m-D3 | A-7 |
| 51379 | m-D3 | H | H | H | m-D3 | A-10 |
| 51380 | m-D3 | H | H | H | m-D3 | A-12 |
| 51381 | m-D3 | H | H | H | m-D3 | A-14 |
| 51382 | m-D3 | H | H | H | m-D3 | A-21 |
| 51383 | m-D3 | H | H | H | m-D3 | A-27 |
| 51384 | m-D3 | H | H | H | m-D3 | A-30 |
| 51385 | m-D3 | H | H | H | m-D3 | A-31 |
| 51386 | m-D3 | H | H | H | m-D3 | A-34 |
| 51387 | m-D3 | H | H | H | m-D3 | A-38 |
| 51388 | m-D3 | H | H | H | m-D3 | A-39 |
| 51389 | m-D3 | H | H | H | m-D3 | A-40 |
| 51390 | m-D3 | H | H | H | m-D3 | A-43 |
| 51391 | m-D3 | H | H | H | m-D3 | A-44 |
| 51392 | m-D3 | H | H | H | m-D3 | A-45 |
| 51393 | m-D4 | H | H | H | m-D4 | A-6 |
| 51394 | m-D4 | H | H | H | m-D4 | A-7 |
| 51395 | m-D4 | H | H | H | m-D4 | A-10 |
| 51396 | m-D4 | H | H | H | m-D4 | A-12 |
| 51397 | m-D4 | H | H | H | m-D4 | A-14 |
| 51398 | m-D4 | H | H | H | m-D4 | A-21 |
| 51399 | m-D4 | H | H | H | m-D4 | A-27 |
| 51400 | m-D4 | H | H | H | m-D4 | A-30 |
| 51401 | m-D4 | H | H | H | m-D4 | A-31 |
| 51402 | m-D4 | H | H | H | m-D4 | A-34 |
| 51403 | m-D4 | H | H | H | m-D4 | A-38 |
| 51404 | m-D4 | H | H | H | m-D4 | A-39 |
| 51405 | m-D4 | H | H | H | m-D4 | A-40 |
| 51406 | m-D4 | H | H | H | m-D4 | A-43 |
| 51407 | m-D4 | H | H | H | m-D4 | A-44 |
| 51408 | m-D4 | H | H | H | m-D4 | A-45 |
| 51409 | m-D5 | H | H | H | m-D5 | A-6 |
| 51410 | m-D5 | H | H | H | m-D5 | A-7 |
| 51411 | m-D5 | H | H | H | m-D5 | A-10 |
| 51412 | m-D5 | H | H | H | m-D5 | A-12 |
| 51413 | m-D5 | H | H | H | m-D5 | A-14 |
| 51414 | m-D5 | H | H | H | m-D5 | A-21 |
| 51415 | m-D5 | H | H | H | m-D5 | A-27 |
| 51416 | m-D5 | H | H | H | m-D5 | A-30 |
| 51417 | m-D5 | H | H | H | m-D5 | A-31 |
| 51418 | m-D5 | H | H | H | m-D5 | A-34 |
| 51419 | m-D5 | H | H | H | m-D5 | A-38 |
| 51420 | m-D5 | H | H | H | m-D5 | A-39 |
| 51421 | m-D5 | H | H | H | m-D5 | A-40 |
| 51422 | m-D5 | H | H | H | m-D5 | A-43 |
| 51423 | m-D5 | H | H | H | m-D5 | A-44 |
| 51424 | m-D5 | H | H | H | m-D5 | A-45 |
| 51425 | m-D6 | H | H | H | m-D6 | A-6 |
| 51426 | m-D6 | H | H | H | m-D6 | A-7 |
| 51427 | m-D6 | H | H | H | m-D6 | A-10 |
| 51428 | m-D6 | H | H | H | m-D6 | A-12 |
| 51429 | m-D6 | H | H | H | m-D6 | A-14 |
| 51430 | m-D6 | H | H | H | m-D6 | A-21 |
| 51431 | m-D6 | H | H | H | m-D6 | A-27 |
| 51432 | m-D6 | H | H | H | m-D6 | A-30 |
| 51433 | m-D6 | H | H | H | m-D6 | A-31 |
| 51434 | m-D6 | H | H | H | m-D6 | A-34 |
| 51435 | m-D6 | H | H | H | m-D6 | A-38 |
| 51436 | m-D6 | H | H | H | m-D6 | A-39 |
| 51437 | m-D6 | H | H | H | m-D6 | A-40 |
| 51438 | m-D6 | H | H | H | m-D6 | A-43 |
| 51439 | m-D6 | H | H | H | m-D6 | A-44 |
| 51440 | m-D6 | H | H | H | m-D6 | A-45 |
| 51441 | m-D7 | H | H | H | m-D7 | A-6 |
| 51442 | m-D7 | H | H | H | m-D7 | A-7 |
| 51443 | m-D7 | H | H | H | m-D7 | A-10 |
| 51444 | m-D7 | H | H | H | m-D7 | A-12 |
| 51445 | m-D7 | H | H | H | m-D7 | A-14 |
| 51446 | m-D7 | H | H | H | m-D7 | A-21 |
| 51447 | m-D7 | H | H | H | m-D7 | A-27 |
| 51448 | m-D7 | H | H | H | m-D7 | A-30 |
| 51449 | m-D7 | H | H | H | m-D7 | A-31 |
| 51450 | m-D7 | H | H | H | m-D7 | A-34 |
| 51451 | m-D7 | H | H | H | m-D7 | A-38 |
| 51452 | m-D7 | H | H | H | m-D7 | A-39 |
| 51453 | m-D7 | H | H | H | m-D7 | A-40 |
| 51454 | m-D7 | H | H | H | m-D7 | A-43 |
| 51455 | m-D7 | H | H | H | m-D7 | A-44 |
| 51456 | m-D7 | H | H | H | m-D7 | A-45 |
| 51457 | m-D8 | H | H | H | m-D8 | A-6 |
| 51458 | m-D8 | H | H | H | m-D8 | A-7 |
| 51459 | m-D8 | H | H | H | m-D8 | A-10 |
| 51460 | m-D8 | H | H | H | m-D8 | A-12 |
| 51461 | m-D8 | H | H | H | m-D8 | A-14 |
| 51462 | m-D8 | H | H | H | m-D8 | A-21 |
| 51463 | m-D8 | H | H | H | m-D8 | A-27 |
| 51464 | m-D8 | H | H | H | m-D8 | A-30 |
| 51465 | m-D8 | H | H | H | m-D8 | A-31 |
| 51466 | m-D8 | H | H | H | m-D8 | A-34 |
| 51467 | m-D8 | H | H | H | m-D8 | A-38 |
| 51468 | m-D8 | H | H | H | m-D8 | A-39 |
| 51469 | m-D8 | H | H | H | m-D8 | A-40 |
| 51470 | m-D8 | H | H | H | m-D8 | A-43 |
| 51471 | m-D8 | H | H | H | m-D8 | A-44 |
| 51472 | m-D8 | H | H | H | m-D8 | A-45 |
| 51473 | m-D9 | H | H | H | m-D9 | A-6 |
| 51474 | m-D9 | H | H | H | m-D9 | A-7 |
| 51475 | m-D9 | H | H | H | m-D9 | A-10 |
| 51476 | m-D9 | H | H | H | m-D9 | A-12 |
| 51477 | m-D9 | H | H | H | m-D9 | A-14 |
| 51478 | m-D9 | H | H | H | m-D9 | A-21 |
| 51479 | m-D9 | H | H | H | m-D9 | A-27 |
| 51480 | m-D9 | H | H | H | m-D9 | A-30 |
| 51481 | m-D9 | H | H | H | m-D9 | A-31 |
| 51482 | m-D9 | H | H | H | m-D9 | A-34 |
| 51483 | m-D9 | H | H | H | m-D9 | A-38 |
| 51484 | m-D9 | H | H | H | m-D9 | A-39 |
| 51485 | m-D9 | H | H | H | m-D9 | A-40 |
| 51486 | m-D9 | H | H | H | m-D9 | A-43 |
| 51487 | m-D9 | H | H | H | m-D9 | A-44 |
| 51488 | m-D9 | H | H | H | m-D9 | A-45 |
| 51489 | m-D10 | H | H | H | m-D10 | A-6 |
| 51490 | m-D10 | H | H | H | m-D10 | A-7 |
| 51491 | m-D10 | H | H | H | m-D10 | A-10 |
| 51492 | m-D10 | H | H | H | m-D10 | A-12 |
| 51493 | m-D10 | H | H | H | m-D10 | A-14 |
| 51494 | m-D10 | H | H | H | m-D10 | A-21 |
| 51495 | m-D10 | H | H | H | m-D10 | A-27 |
| 51496 | m-D10 | H | H | H | m-D10 | A-30 |
| 51497 | m-D10 | H | H | H | m-D10 | A-31 |
| 51498 | m-D10 | H | H | H | m-D10 | A-34 |
| 51499 | m-D10 | H | H | H | m-D10 | A-38 |
| 51500 | m-D10 | H | H | H | m-D10 | A-39 |
| 51501 | m-D10 | H | H | H | m-D10 | A-40 |
| 51502 | m-D10 | H | H | H | m-D10 | A-43 |
| 51503 | m-D10 | H | H | H | m-D10 | A-44 |
| 51504 | m-D10 | H | H | H | m-D10 | A-45 |
| 51505 | m-D11 | H | H | H | m-D11 | A-6 |
| 51506 | m-D11 | H | H | H | m-D11 | A-7 |
| 51507 | m-D11 | H | H | H | m-D11 | A-10 |
| 51508 | m-D11 | H | H | H | m-D11 | A-12 |
| 51509 | m-D11 | H | H | H | m-D11 | A-14 |
| 51510 | m-D11 | H | H | H | m-D11 | A-21 |
| 51511 | m-D11 | H | H | H | m-D11 | A-27 |
| 51512 | m-D11 | H | H | H | m-D11 | A-30 |
| 51513 | m-D11 | H | H | H | m-D11 | A-31 |
| 51514 | m-D11 | H | H | H | m-D11 | A-34 |
| 51515 | m-D11 | H | H | H | m-D11 | A-38 |
| 51516 | m-D11 | H | H | H | m-D11 | A-39 |
| 51517 | m-D11 | H | H | H | m-D11 | A-40 |
| 51518 | m-D11 | H | H | H | m-D11 | A-43 |
| 51519 | m-D11 | H | H | H | m-D11 | A-44 |
| 51520 | m-D11 | H | H | H | m-D11 | A-45 |
| 51521 | m-D12 | H | H | H | m-D12 | A-6 |
| 51522 | m-D12 | H | H | H | m-D12 | A-7 |
| 51523 | m-D12 | H | H | H | m-D12 | A-10 |
| 51524 | m-D12 | H | H | H | m-D12 | A-12 |
| 51525 | m-D12 | H | H | H | m-D12 | A-14 |
| 51526 | m-D12 | H | H | H | m-D12 | A-21 |
| 51527 | m-D12 | H | H | H | m-D12 | A-27 |
| 51528 | m-D12 | H | H | H | m-D12 | A-30 |
| 51529 | m-D12 | H | H | H | m-D12 | A-31 |
| 51530 | m-D12 | H | H | H | m-D12 | A-34 |
| 51531 | m-D12 | H | H | H | m-D12 | A-38 |
| 51532 | m-D12 | H | H | H | m-D12 | A-39 |
| 51533 | m-D12 | H | H | H | m-D12 | A-40 |
| 51534 | m-D12 | H | H | H | m-D12 | A-43 |
| 51535 | m-D12 | H | H | H | m-D12 | A-44 |
| 51536 | m-D12 | H | H | H | m-D12 | A-45 |
| 51537 | m-D13 | H | H | H | m-D13 | A-6 |
| 51538 | m-D13 | H | H | H | m-D13 | A-7 |
| 51539 | m-D13 | H | H | H | m-D13 | A-10 |
| 51540 | m-D13 | H | H | H | m-D13 | A-12 |
| 51541 | m-D13 | H | H | H | m-D13 | A-14 |
| 51542 | m-D13 | H | H | H | m-D13 | A-21 |
| 51543 | m-D13 | H | H | H | m-D13 | A-27 |
| 51544 | m-D13 | H | H | H | m-D13 | A-30 |
| 51545 | m-D13 | H | H | H | m-D13 | A-31 |
| 51546 | m-D13 | H | H | H | m-D13 | A-34 |
| 51547 | m-D13 | H | H | H | m-D13 | A-38 |
| 51548 | m-D13 | H | H | H | m-D13 | A-39 |
| 51549 | m-D13 | H | H | H | m-D13 | A-40 |
| 51550 | m-D13 | H | H | H | m-D13 | A-43 |
| 51551 | m-D13 | H | H | H | m-D13 | A-44 |
| 51552 | m-D13 | H | H | H | m-D13 | A-45 |
| 51553 | m-D14 | H | H | H | m-D14 | A-6 |
| 51554 | m-D14 | H | H | H | m-D14 | A-7 |
| 51555 | m-D14 | H | H | H | m-D14 | A-10 |
| 51556 | m-D14 | H | H | H | m-D14 | A-12 |
| 51557 | m-D14 | H | H | H | m-D14 | A-14 |
| 51558 | m-D14 | H | H | H | m-D14 | A-21 |
| 51559 | m-D14 | H | H | H | m-D14 | A-27 |
| 51560 | m-D14 | H | H | H | m-D14 | A-30 |
| 51561 | m-D14 | H | H | H | m-D14 | A-31 |
| 51562 | m-D14 | H | H | H | m-D14 | A-34 |
| 51563 | m-D14 | H | H | H | m-D14 | A-38 |
| 51564 | m-D14 | H | H | H | m-D14 | A-39 |
| 51565 | m-D14 | H | H | H | m-D14 | A-40 |
| 51566 | m-D14 | H | H | H | m-D14 | A-43 |
| 51567 | m-D14 | H | H | H | m-D14 | A-44 |
| 51568 | m-D14 | H | H | H | m-D14 | A-45 |
| 51569 | H | m-D1 | m-D1 | H | H | A-6 |
| 51570 | H | m-D1 | m-D1 | H | H | A-7 |
| 51571 | H | m-D1 | m-D1 | H | H | A-10 |
| 51572 | H | m-D1 | m-D1 | H | H | A-12 |
| 51573 | H | m-D1 | m-D1 | H | H | A-14 |
| 51574 | H | m-D1 | m-D1 | H | H | A-21 |
| 51575 | H | m-D1 | m-D1 | H | H | A-27 |
| 51576 | H | m-D1 | m-D1 | H | H | A-30 |
| 51577 | H | m-D1 | m-D1 | H | H | A-31 |
| 51578 | H | m-D1 | m-D1 | H | H | A-34 |
| 51579 | H | m-D1 | m-D1 | H | H | A-38 |
| 51580 | H | m-D1 | m-D1 | H | H | A-39 |
| 51581 | H | m-D1 | m-D1 | H | H | A-40 |
| 51582 | H | m-D1 | m-D1 | H | H | A-43 |
| 51583 | H | m-D1 | m-D1 | H | H | A-44 |
| 51584 | H | m-D1 | m-D1 | H | H | A-45 |
| 51585 | H | m-D2 | m-D2 | H | H | A-6 |
| 51586 | H | m-D2 | m-D2 | H | H | A-7 |
| 51587 | H | m-D2 | m-D2 | H | H | A-10 |
| 51588 | H | m-D2 | m-D2 | H | H | A-12 |
| 51589 | H | m-D2 | m-D2 | H | H | A-14 |
| 51590 | H | m-D2 | m-D2 | H | H | A-21 |
| 51591 | H | m-D2 | m-D2 | H | H | A-27 |
| 51592 | H | m-D2 | m-D2 | H | H | A-30 |
| 51593 | H | m-D2 | m-D2 | H | H | A-31 |
| 51594 | H | m-D2 | m-D2 | H | H | A-34 |
| 51595 | H | m-D2 | m-D2 | H | H | A-38 |
| 51596 | H | m-D2 | m-D2 | H | H | A-39 |
| 51597 | H | m-D2 | m-D2 | H | H | A-40 |
| 51598 | H | m-D2 | m-D2 | H | H | A-43 |
| 51599 | H | m-D2 | m-D2 | H | H | A-44 |
| 51600 | H | m-D2 | m-D2 | H | H | A-45 |
| 51601 | H | m-D3 | m-D3 | H | H | A-6 |
| 51602 | H | m-D3 | m-D3 | H | H | A-7 |
| 51603 | H | m-D3 | m-D3 | H | H | A-10 |
| 51604 | H | m-D3 | m-D3 | H | H | A-12 |
| 51605 | H | m-D3 | m-D3 | H | H | A-14 |
| 51606 | H | m-D3 | m-D3 | H | H | A-21 |
| 51607 | H | m-D3 | m-D3 | H | H | A-27 |
| 51608 | H | m-D3 | m-D3 | H | H | A-30 |
| 51609 | H | m-D3 | m-D3 | H | H | A-31 |
| 51610 | H | m-D3 | m-D3 | H | H | A-34 |
| 51611 | H | m-D3 | m-D3 | H | H | A-38 |
| 51612 | H | m-D3 | m-D3 | H | H | A-39 |
| 51613 | H | m-D3 | m-D3 | H | H | A-40 |
| 51614 | H | m-D3 | m-D3 | H | H | A-43 |
| 51615 | H | m-D3 | m-D3 | H | H | A-44 |
| 51616 | H | m-D3 | m-D3 | H | H | A-45 |
| 51617 | H | m-D4 | m-D4 | H | H | A-6 |
| 51618 | H | m-D4 | m-D4 | H | H | A-7 |
| 51619 | H | m-D4 | m-D4 | H | H | A-10 |
| 51620 | H | m-D4 | m-D4 | H | H | A-12 |
| 51621 | H | m-D4 | m-D4 | H | H | A-14 |
| 51622 | H | m-D4 | m-D4 | H | H | A-21 |
| 51623 | H | m-D4 | m-D4 | H | H | A-27 |
| 51624 | H | m-D4 | m-D4 | H | H | A-30 |
| 51625 | H | m-D4 | m-D4 | H | H | A-31 |
| 51626 | H | m-D4 | m-D4 | H | H | A-34 |
| 51627 | H | m-D4 | m-D4 | H | H | A-38 |
| 51628 | H | m-D4 | m-D4 | H | H | A-39 |
| 51629 | H | m-D4 | m-D4 | H | H | A-40 |
| 51630 | H | m-D4 | m-D4 | H | H | A-43 |
| 51631 | H | m-D4 | m-D4 | H | H | A-44 |
| 51632 | H | m-D4 | m-D4 | H | H | A-45 |
| 51633 | H | m-D5 | m-D5 | H | H | A-6 |
| 51634 | H | m-D5 | m-D5 | H | H | A-7 |
| 51635 | H | m-D5 | m-D5 | H | H | A-10 |
| 51636 | H | m-D5 | m-D5 | H | H | A-12 |
| 51637 | H | m-D5 | m-D5 | H | H | A-14 |
| 51638 | H | m-D5 | m-D5 | H | H | A-21 |
| 51639 | H | m-D5 | m-D5 | H | H | A-27 |
| 51640 | H | m-D5 | m-D5 | H | H | A-30 |
| 51641 | H | m-D5 | m-D5 | H | H | A-31 |
| 51642 | H | m-D5 | m-D5 | H | H | A-34 |
| 51643 | H | m-D5 | m-D5 | H | H | A-38 |
| 51644 | H | m-D5 | m-D5 | H | H | A-39 |
| 51645 | H | m-D5 | m-D5 | H | H | A-40 |
| 51646 | H | m-D5 | m-D5 | H | H | A-43 |
| 51647 | H | m-D5 | m-D5 | H | H | A-44 |
| 51648 | H | m-D5 | m-D5 | H | H | A-45 |
| 51649 | H | m-D6 | m-D6 | H | H | A-6 |
| 51650 | H | m-D6 | m-D6 | H | H | A-7 |
| 51651 | H | m-D6 | m-D6 | H | H | A-10 |
| 51652 | H | m-D6 | m-D6 | H | H | A-12 |
| 51653 | H | m-D6 | m-D6 | H | H | A-14 |
| 51654 | H | m-D6 | m-D6 | H | H | A-21 |
| 51655 | H | m-D6 | m-D6 | H | H | A-27 |
| 51656 | H | m-D6 | m-D6 | H | H | A-30 |
| 51657 | H | m-D6 | m-D6 | H | H | A-31 |
| 51658 | H | m-D6 | m-D6 | H | H | A-34 |
| 51659 | H | m-D6 | m-D6 | H | H | A-38 |
| 51660 | H | m-D6 | m-D6 | H | H | A-39 |
| 51661 | H | m-D6 | m-D6 | H | H | A-40 |
| 51662 | H | m-D6 | m-D6 | H | H | A-43 |
| 51663 | H | m-D6 | m-D6 | H | H | A-44 |
| 51664 | H | m-D6 | m-D6 | H | H | A-45 |
| 51665 | H | m-D7 | m-D7 | H | H | A-6 |
| 51666 | H | m-D7 | m-D7 | H | H | A-7 |
| 51667 | H | m-D7 | m-D7 | H | H | A-10 |
| 51668 | H | m-D7 | m-D7 | H | H | A-12 |
| 51669 | H | m-D7 | m-D7 | H | H | A-14 |
| 51670 | H | m-D7 | m-D7 | H | H | A-21 |
| 51671 | H | m-D7 | m-D7 | H | H | A-27 |
| 51672 | H | m-D7 | m-D7 | H | H | A-30 |
| 51673 | H | m-D7 | m-D7 | H | H | A-31 |
| 51674 | H | m-D7 | m-D7 | H | H | A-34 |
| 51675 | H | m-D7 | m-D7 | H | H | A-38 |
| 51676 | H | m-D7 | m-D7 | H | H | A-39 |
| 51677 | H | m-D7 | m-D7 | H | H | A-40 |
| 51678 | H | m-D7 | m-D7 | H | H | A-43 |
| 51679 | H | m-D7 | m-D7 | H | H | A-44 |
| 51680 | H | m-D7 | m-D7 | H | H | A-45 |
| 51681 | H | m-D8 | m-D8 | H | H | A-6 |
| 51682 | H | m-D8 | m-D8 | H | H | A-7 |
| 51683 | H | m-D8 | m-D8 | H | H | A-10 |
| 51684 | H | m-D8 | m-D8 | H | H | A-12 |
| 51685 | H | m-D8 | m-D8 | H | H | A-14 |
| 51686 | H | m-D8 | m-D8 | H | H | A-21 |
| 51687 | H | m-D8 | m-D8 | H | H | A-27 |
| 51688 | H | m-D8 | m-D8 | H | H | A-30 |
| 51689 | H | m-D8 | m-D8 | H | H | A-31 |
| 51690 | H | m-D8 | m-D8 | H | H | A-34 |
| 51691 | H | m-D8 | m-D8 | H | H | A-38 |
| 51692 | H | m-D8 | m-D8 | H | H | A-39 |
| 51693 | H | m-D8 | m-D8 | H | H | A-40 |
| 51694 | H | m-D8 | m-D8 | H | H | A-43 |
| 51695 | H | m-D8 | m-D8 | H | H | A-44 |
| 51696 | H | m-D8 | m-D8 | H | H | A-45 |
| 51697 | H | m-D9 | m-D9 | H | H | A-6 |
| 51698 | H | m-D9 | m-D9 | H | H | A-7 |
| 51699 | H | m-D9 | m-D9 | H | H | A-10 |
| 51700 | H | m-D9 | m-D9 | H | H | A-12 |
| 51701 | H | m-D9 | m-D9 | H | H | A-14 |
| 51702 | H | m-D9 | m-D9 | H | H | A-21 |
| 51703 | H | m-D9 | m-D9 | H | H | A-27 |
| 51704 | H | m-D9 | m-D9 | H | H | A-30 |
| 51705 | H | m-D9 | m-D9 | H | H | A-31 |
| 51706 | H | m-D9 | m-D9 | H | H | A-34 |
| 51707 | H | m-D9 | m-D9 | H | H | A-38 |
| 51708 | H | m-D9 | m-D9 | H | H | A-39 |
| 51709 | H | m-D9 | m-D9 | H | H | A-40 |
| 51710 | H | m-D9 | m-D9 | H | H | A-43 |
| 51711 | H | m-D9 | m-D9 | H | H | A-44 |
| 51712 | H | m-D9 | m-D9 | H | H | A-45 |
| 51713 | H | m-D10 | m-D10 | H | H | A-6 |
| 51714 | H | m-D10 | m-D10 | H | H | A-7 |
| 51715 | H | m-D10 | m-D10 | H | H | A-10 |
| 51716 | H | m-D10 | m-D10 | H | H | A-12 |
| 51717 | H | m-D10 | m-D10 | H | H | A-14 |
| 51718 | H | m-D10 | m-D10 | H | H | A-21 |
| 51719 | H | m-D10 | m-D10 | H | H | A-27 |
| 51720 | H | m-D10 | m-D10 | H | H | A-30 |
| 51721 | H | m-D10 | m-D10 | H | H | A-31 |
| 51722 | H | m-D10 | m-D10 | H | H | A-34 |
| 51723 | H | m-D10 | m-D10 | H | H | A-38 |
| 51724 | H | m-D10 | m-D10 | H | H | A-39 |
| 51725 | H | m-D10 | m-D10 | H | H | A-40 |
| 51726 | H | m-D10 | m-D10 | H | H | A-43 |
| 51727 | H | m-D10 | m-D10 | H | H | A-44 |
| 51728 | H | m-D10 | m-D10 | H | H | A-45 |
| 51729 | H | m-D11 | m-D11 | H | H | A-6 |
| 51730 | H | m-D11 | m-D11 | H | H | A-7 |
| 51731 | H | m-D11 | m-D11 | H | H | A-10 |
| 51732 | H | m-D11 | m-D11 | H | H | A-12 |
| 51733 | H | m-D11 | m-D11 | H | H | A-14 |
| 51734 | H | m-D11 | m-D11 | H | H | A-21 |
| 51735 | H | m-D11 | m-D11 | H | H | A-27 |
| 51736 | H | m-D11 | m-D11 | H | H | A-30 |
| 51737 | H | m-D11 | m-D11 | H | H | A-31 |
| 51738 | H | m-D11 | m-D11 | H | H | A-34 |
| 51739 | H | m-D11 | m-D11 | H | H | A-38 |
| 51740 | H | m-D11 | m-D11 | H | H | A-39 |
| 51741 | H | m-D11 | m-D11 | H | H | A-40 |
| 51742 | H | m-D11 | m-D11 | H | H | A-43 |
| 51743 | H | m-D11 | m-D11 | H | H | A-44 |
| 51744 | H | m-D11 | m-D11 | H | H | A-45 |
| 51745 | H | m-D12 | m-D12 | H | H | A-6 |
| 51746 | H | m-D12 | m-D12 | H | H | A-7 |
| 51747 | H | m-D12 | m-D12 | H | H | A-10 |
| 51748 | H | m-D12 | m-D12 | H | H | A-12 |
| 51749 | H | m-D12 | m-D12 | H | H | A-14 |
| 51750 | H | m-D12 | m-D12 | H | H | A-21 |
| 51751 | H | m-D12 | m-D12 | H | H | A-27 |
| 51752 | H | m-D12 | m-D12 | H | H | A-30 |
| 51753 | H | m-D12 | m-D12 | H | H | A-31 |
| 51754 | H | m-D12 | m-D12 | H | H | A-34 |
| 51755 | H | m-D12 | m-D12 | H | H | A-38 |
| 51756 | H | m-D12 | m-D12 | H | H | A-39 |
| 51757 | H | m-D12 | m-D12 | H | H | A-40 |
| 51758 | H | m-D12 | m-D12 | H | H | A-43 |
| 51759 | H | m-D12 | m-D12 | H | H | A-44 |
| 51760 | H | m-D12 | m-D12 | H | H | A-45 |
| 51761 | H | m-D13 | m-D13 | H | H | A-6 |
| 51762 | H | m-D13 | m-D13 | H | H | A-7 |
| 51763 | H | m-D13 | m-D13 | H | H | A-10 |
| 51764 | H | m-D13 | m-D13 | H | H | A-12 |
| 51765 | H | m-D13 | m-D13 | H | H | A-14 |
| 51766 | H | m-D13 | m-D13 | H | H | A-21 |
| 51767 | H | m-D13 | m-D13 | H | H | A-27 |
| 51768 | H | m-D13 | m-D13 | H | H | A-30 |
| 51769 | H | m-D13 | m-D13 | H | H | A-31 |
| 51770 | H | m-D13 | m-D13 | H | H | A-34 |
| 51771 | H | m-D13 | m-D13 | H | H | A-38 |
| 51772 | H | m-D13 | m-D13 | H | H | A-39 |
| 51773 | H | m-D13 | m-D13 | H | H | A-40 |
| 51774 | H | m-D13 | m-D13 | H | H | A-43 |
| 51775 | H | m-D13 | m-D13 | H | H | A-44 |
| 51776 | H | m-D13 | m-D13 | H | H | A-45 |
| 51777 | H | m-D14 | m-D14 | H | H | A-6 |
| 51778 | H | m-D14 | m-D14 | H | H | A-7 |
| 51779 | H | m-D14 | m-D14 | H | H | A-10 |
| 51780 | H | m-D14 | m-D14 | H | H | A-12 |
| 51781 | H | m-D14 | m-D14 | H | H | A-14 |
| 51782 | H | m-D14 | m-D14 | H | H | A-21 |
| 51783 | H | m-D14 | m-D14 | H | H | A-27 |
| 51784 | H | m-D14 | m-D14 | H | H | A-30 |
| 51785 | H | m-D14 | m-D14 | H | H | A-31 |
| 51786 | H | m-D14 | m-D14 | H | H | A-34 |
| 51787 | H | m-D14 | m-D14 | H | H | A-38 |
| 51788 | H | m-D14 | m-D14 | H | H | A-39 |
| 51789 | H | m-D14 | m-D14 | H | H | A-40 |
| 51790 | H | m-D14 | m-D14 | H | H | A-43 |
| 51791 | H | m-D14 | m-D14 | H | H | A-44 |
| 51792 | H | m-D14 | m-D14 | H | H | A-45 |
| 51793 | H | m-D1 | H | m-D1 | H | A-6 |
| 51794 | H | m-D1 | H | m-D1 | H | A-7 |
| 51795 | H | m-D1 | H | m-D1 | H | A-10 |
| 51796 | H | m-D1 | H | m-D1 | H | A-12 |
| 51797 | H | m-D1 | H | m-D1 | H | A-14 |
| 51798 | H | m-D1 | H | m-D1 | H | A-21 |
| 51799 | H | m-D1 | H | m-D1 | H | A-27 |
| 51800 | H | m-D1 | H | m-D1 | H | A-30 |
| 51801 | H | m-D1 | H | m-D1 | H | A-31 |
| 51802 | H | m-D1 | H | m-D1 | H | A-34 |
| 51803 | H | m-D1 | H | m-D1 | H | A-38 |
| 51804 | H | m-D1 | H | m-D1 | H | A-39 |
| 51805 | H | m-D1 | H | m-D1 | H | A-40 |
| 51806 | H | m-D1 | H | m-D1 | H | A-43 |
| 51807 | H | m-D1 | H | m-D1 | H | A-44 |
| 51808 | H | m-D1 | H | m-D1 | H | A-45 |
| 51809 | H | m-D2 | H | m-D2 | H | A-6 |
| 51810 | H | m-D2 | H | m-D2 | H | A-7 |
| 51811 | H | m-D2 | H | m-D2 | H | A-10 |
| 51812 | H | m-D2 | H | m-D2 | H | A-12 |
| 51813 | H | m-D2 | H | m-D2 | H | A-14 |
| 51814 | H | m-D2 | H | m-D2 | H | A-21 |
| 51815 | H | m-D2 | H | m-D2 | H | A-27 |
| 51816 | H | m-D2 | H | m-D2 | H | A-30 |
| 51817 | H | m-D2 | H | m-D2 | H | A-31 |
| 51818 | H | m-D2 | H | m-D2 | H | A-34 |
| 51819 | H | m-D2 | H | m-D2 | H | A-38 |
| 51820 | H | m-D2 | H | m-D2 | H | A-39 |
| 51821 | H | m-D2 | H | m-D2 | H | A-40 |
| 51822 | H | m-D2 | H | m-D2 | H | A-43 |
| 51823 | H | m-D2 | H | m-D2 | H | A-44 |
| 51824 | H | m-D2 | H | m-D2 | H | A-45 |
| 51825 | H | m-D3 | H | m-D3 | H | A-6 |
| 51826 | H | m-D3 | H | m-D3 | H | A-7 |
| 51827 | H | m-D3 | H | m-D3 | H | A-10 |
| 51828 | H | m-D3 | H | m-D3 | H | A-12 |
| 51829 | H | m-D3 | H | m-D3 | H | A-14 |
| 51830 | H | m-D3 | H | m-D3 | H | A-21 |
| 51831 | H | m-D3 | H | m-D3 | H | A-27 |
| 51832 | H | m-D3 | H | m-D3 | H | A-30 |
| 51833 | H | m-D3 | H | m-D3 | H | A-31 |
| 51834 | H | m-D3 | H | m-D3 | H | A-34 |
| 51835 | H | m-D3 | H | m-D3 | H | A-38 |
| 51836 | H | m-D3 | H | m-D3 | H | A-39 |
| 51837 | H | m-D3 | H | m-D3 | H | A-40 |
| 51838 | H | m-D3 | H | m-D3 | H | A-43 |
| 51839 | H | m-D3 | H | m-D3 | H | A-44 |
| 51840 | H | m-D3 | H | m-D3 | H | A-45 |
| 51841 | H | m-D4 | H | m-D4 | H | A-6 |
| 51842 | H | m-D4 | H | m-D4 | H | A-7 |
| 51843 | H | m-D4 | H | m-D4 | H | A-10 |
| 51844 | H | m-D4 | H | m-D4 | H | A-12 |
| 51845 | H | m-D4 | H | m-D4 | H | A-14 |
| 51846 | H | m-D4 | H | m-D4 | H | A-21 |
| 51847 | H | m-D4 | H | m-D4 | H | A-27 |
| 51848 | H | m-D4 | H | m-D4 | H | A-30 |
| 51849 | H | m-D4 | H | m-D4 | H | A-31 |
| 51850 | H | m-D4 | H | m-D4 | H | A-34 |
| 51851 | H | m-D4 | H | m-D4 | H | A-38 |
| 51852 | H | m-D4 | H | m-D4 | H | A-39 |
| 51853 | H | m-D4 | H | m-D4 | H | A-40 |
| 51854 | H | m-D4 | H | m-D4 | H | A-43 |
| 51855 | H | m-D4 | H | m-D4 | H | A-44 |
| 51856 | H | m-D4 | H | m-D4 | H | A-45 |
| 51857 | H | m-D5 | H | m-D5 | H | A-6 |
| 51858 | H | m-D5 | H | m-D5 | H | A-7 |
| 51859 | H | m-D5 | H | m-D5 | H | A-10 |
| 51860 | H | m-D5 | H | m-D5 | H | A-12 |
| 51861 | H | m-D5 | H | m-D5 | H | A-14 |
| 51862 | H | m-D5 | H | m-D5 | H | A-21 |
| 51863 | H | m-D5 | H | m-D5 | H | A-27 |
| 51864 | H | m-D5 | H | m-D5 | H | A-30 |
| 51865 | H | m-D5 | H | m-D5 | H | A-31 |
| 51866 | H | m-D5 | H | m-D5 | H | A-34 |
| 51867 | H | m-D5 | H | m-D5 | H | A-38 |
| 51868 | H | m-D5 | H | m-D5 | H | A-39 |
| 51869 | H | m-D5 | H | m-D5 | H | A-40 |
| 51870 | H | m-D5 | H | m-D5 | H | A-43 |
| 51871 | H | m-D5 | H | m-D5 | H | A-44 |
| 51872 | H | m-D5 | H | m-D5 | H | A-45 |
| 51873 | H | m-D6 | H | m-D6 | H | A-6 |
| 51874 | H | m-D6 | H | m-D6 | H | A-7 |
| 51875 | H | m-D6 | H | m-D6 | H | A-10 |
| 51876 | H | m-D6 | H | m-D6 | H | A-12 |
| 51877 | H | m-D6 | H | m-D6 | H | A-14 |
| 51878 | H | m-D6 | H | m-D6 | H | A-21 |
| 51879 | H | m-D6 | H | m-D6 | H | A-27 |
| 51880 | H | m-D6 | H | m-D6 | H | A-30 |
| 51881 | H | m-D6 | H | m-D6 | H | A-31 |
| 51882 | H | m-D6 | H | m-D6 | H | A-34 |
| 51883 | H | m-D6 | H | m-D6 | H | A-38 |
| 51884 | H | m-D6 | H | m-D6 | H | A-39 |
| 51885 | H | m-D6 | H | m-D6 | H | A-40 |
| 51886 | H | m-D6 | H | m-D6 | H | A-43 |
| 51887 | H | m-D6 | H | m-D6 | H | A-44 |
| 51888 | H | m-D6 | H | m-D6 | H | A-45 |
| 51889 | H | m-D7 | H | m-D7 | H | A-6 |
| 51890 | H | m-D7 | H | m-D7 | H | A-7 |
| 51891 | H | m-D7 | H | m-D7 | H | A-10 |
| 51892 | H | m-D7 | H | m-D7 | H | A-12 |
| 51893 | H | m-D7 | H | m-D7 | H | A-14 |
| 51894 | H | m-D7 | H | m-D7 | H | A-21 |
| 51895 | H | m-D7 | H | m-D7 | H | A-27 |
| 51896 | H | m-D7 | H | m-D7 | H | A-30 |
| 51897 | H | m-D7 | H | m-D7 | H | A-31 |
| 51898 | H | m-D7 | H | m-D7 | H | A-34 |
| 51899 | H | m-D7 | H | m-D7 | H | A-38 |
| 51900 | H | m-D7 | H | m-D7 | H | A-39 |
| 51901 | H | m-D7 | H | m-D7 | H | A-40 |
| 51902 | H | m-D7 | H | m-D7 | H | A-43 |
| 51903 | H | m-D7 | H | m-D7 | H | A-44 |
| 51904 | H | m-D7 | H | m-D7 | H | A-45 |
| 51905 | H | m-D8 | H | m-D8 | H | A-6 |
| 51906 | H | m-D8 | H | m-D8 | H | A-7 |
| 51907 | H | m-D8 | H | m-D8 | H | A-10 |
| 51908 | H | m-D8 | H | m-D8 | H | A-12 |
| 51909 | H | m-D8 | H | m-D8 | H | A-14 |
| 51910 | H | m-D8 | H | m-D8 | H | A-21 |
| 51911 | H | m-D8 | H | m-D8 | H | A-27 |
| 51912 | H | m-D8 | H | m-D8 | H | A-30 |
| 51913 | H | m-D8 | H | m-D8 | H | A-31 |
| 51914 | H | m-D8 | H | m-D8 | H | A-34 |
| 51915 | H | m-D8 | H | m-D8 | H | A-38 |
| 51916 | H | m-D8 | H | m-D8 | H | A-39 |
| 51917 | H | m-D8 | H | m-D8 | H | A-40 |
| 51918 | H | m-D8 | H | m-D8 | H | A-43 |
| 51919 | H | m-D8 | H | m-D8 | H | A-44 |
| 51920 | H | m-D8 | H | m-D8 | H | A-45 |
| 51921 | H | m-D9 | H | m-D9 | H | A-6 |
| 51922 | H | m-D9 | H | m-D9 | H | A-7 |
| 51923 | H | m-D9 | H | m-D9 | H | A-10 |
| 51924 | H | m-D9 | H | m-D9 | H | A-12 |
| 51925 | H | m-D9 | H | m-D9 | H | A-14 |
| 51926 | H | m-D9 | H | m-D9 | H | A-21 |
| 51927 | H | m-D9 | H | m-D9 | H | A-27 |
| 51928 | H | m-D9 | H | m-D9 | H | A-30 |
| 51929 | H | m-D9 | H | m-D9 | H | A-31 |
| 51930 | H | m-D9 | H | m-D9 | H | A-34 |
| 51931 | H | m-D9 | H | m-D9 | H | A-38 |
| 51932 | H | m-D9 | H | m-D9 | H | A-39 |
| 51933 | H | m-D9 | H | m-D9 | H | A-40 |
| 51934 | H | m-D9 | H | m-D9 | H | A-43 |
| 51935 | H | m-D9 | H | m-D9 | H | A-44 |
| 51936 | H | m-D9 | H | m-D9 | H | A-45 |
| 51937 | H | m-D10 | H | m-D10 | H | A-6 |
| 51938 | H | m-D10 | H | m-D10 | H | A-7 |
| 51939 | H | m-D10 | H | m-D10 | H | A-10 |
| 51940 | H | m-D10 | H | m-D10 | H | A-12 |
| 51941 | H | m-D10 | H | m-D10 | H | A-14 |
| 51942 | H | m-D10 | H | m-D10 | H | A-21 |
| 51943 | H | m-D10 | H | m-D10 | H | A-27 |
| 51944 | H | m-D10 | H | m-D10 | H | A-30 |
| 51945 | H | m-D10 | H | m-D10 | H | A-31 |
| 51946 | H | m-D10 | H | m-D10 | H | A-34 |
| 51947 | H | m-D10 | H | m-D10 | H | A-38 |
| 51948 | H | m-D10 | H | m-D10 | H | A-39 |
| 51949 | H | m-D10 | H | m-D10 | H | A-40 |
| 51950 | H | m-D10 | H | m-D10 | H | A-43 |
| 51951 | H | m-D10 | H | m-D10 | H | A-44 |
| 51952 | H | m-D10 | H | m-D10 | H | A-45 |
| 51953 | H | m-D11 | H | m-D11 | H | A-6 |
| 51954 | H | m-D11 | H | m-D11 | H | A-7 |
| 51955 | H | m-D11 | H | m-D11 | H | A-10 |
| 51956 | H | m-D11 | H | m-D11 | H | A-12 |
| 51957 | H | m-D11 | H | m-D11 | H | A-14 |
| 51958 | H | m-D11 | H | m-D11 | H | A-21 |
| 51959 | H | m-D11 | H | m-D11 | H | A-27 |
| 51960 | H | m-D11 | H | m-D11 | H | A-30 |
| 51961 | H | m-D11 | H | m-D11 | H | A-31 |
| 51962 | H | m-D11 | H | m-D11 | H | A-34 |
| 51963 | H | m-D11 | H | m-D11 | H | A-38 |
| 51964 | H | m-D11 | H | m-D11 | H | A-39 |
| 51965 | H | m-D11 | H | m-D11 | H | A-40 |
| 51966 | H | m-D11 | H | m-D11 | H | A-43 |
| 51967 | H | m-D11 | H | m-D11 | H | A-44 |
| 51968 | H | m-D11 | H | m-D11 | H | A-45 |
| 51969 | H | m-D12 | H | m-D12 | H | A-6 |
| 51970 | H | m-D12 | H | m-D12 | H | A-7 |
| 51971 | H | m-D12 | H | m-D12 | H | A-10 |
| 51972 | H | m-D12 | H | m-D12 | H | A-12 |
| 51973 | H | m-D12 | H | m-D12 | H | A-14 |
| 51974 | H | m-D12 | H | m-D12 | H | A-21 |
| 51975 | H | m-D12 | H | m-D12 | H | A-27 |
| 51976 | H | m-D12 | H | m-D12 | H | A-30 |
| 51977 | H | m-D12 | H | m-D12 | H | A-31 |
| 51978 | H | m-D12 | H | m-D12 | H | A-34 |
| 51979 | H | m-D12 | H | m-D12 | H | A-38 |
| 51980 | H | m-D12 | H | m-D12 | H | A-39 |
| 51981 | H | m-D12 | H | m-D12 | H | A-40 |
| 51982 | H | m-D12 | H | m-D12 | H | A-43 |
| 51983 | H | m-D12 | H | m-D12 | H | A-44 |
| 51984 | H | m-D12 | H | m-D12 | H | A-45 |
| 51985 | H | m-D13 | H | m-D13 | H | A-6 |
| 51986 | H | m-D13 | H | m-D13 | H | A-7 |
| 51987 | H | m-D13 | H | m-D13 | H | A-10 |
| 51988 | H | m-D13 | H | m-D13 | H | A-12 |
| 51989 | H | m-D13 | H | m-D13 | H | A-14 |
| 51990 | H | m-D13 | H | m-D13 | H | A-21 |
| 51991 | H | m-D13 | H | m-D13 | H | A-27 |
| 51992 | H | m-D13 | H | m-D13 | H | A-30 |
| 51993 | H | m-D13 | H | m-D13 | H | A-31 |
| 51994 | H | m-D13 | H | m-D13 | H | A-34 |
| 51995 | H | m-D13 | H | m-D13 | H | A-38 |
| 51996 | H | m-D13 | H | m-D13 | H | A-39 |
| 51997 | H | m-D13 | H | m-D13 | H | A-40 |
| 51998 | H | m-D13 | H | m-D13 | H | A-43 |
| 51999 | H | m-D13 | H | m-D13 | H | A-44 |
| 52000 | H | m-D13 | H | m-D13 | H | A-45 |
| 52001 | H | m-D14 | H | m-D14 | H | A-6 |
| 52002 | H | m-D14 | H | m-D14 | H | A-7 |
| 52003 | H | m-D14 | H | m-D14 | H | A-10 |
| 52004 | H | m-D14 | H | m-D14 | H | A-12 |
| 52005 | H | m-D14 | H | m-D14 | H | A-14 |
| 52006 | H | m-D14 | H | m-D14 | H | A-21 |
| 52007 | H | m-D14 | H | m-D14 | H | A-27 |
| 52008 | H | m-D14 | H | m-D14 | H | A-30 |
| 52009 | H | m-D14 | H | m-D14 | H | A-31 |
| 52010 | H | m-D14 | H | m-D14 | H | A-34 |
| 52011 | H | m-D14 | H | m-D14 | H | A-38 |
| 52012 | H | m-D14 | H | m-D14 | H | A-39 |
| 52013 | H | m-D14 | H | m-D14 | H | A-40 |
| 52014 | H | m-D14 | H | m-D14 | H | A-43 |
| 52015 | H | m-D14 | H | m-D14 | H | A-44 |
| 52016 | H | m-D14 | H | m-D14 | H | A-45 |
| 52017 | m-D1 | m-D1 | m-D1 | H | H | A-6 |
| 52018 | m-D1 | m-D1 | m-D1 | H | H | A-7 |
| 52019 | m-D1 | m-D1 | m-D1 | H | H | A-10 |
| 52020 | m-D1 | m-D1 | m-D1 | H | H | A-12 |
| 52021 | m-D1 | m-D1 | m-D1 | H | H | A-14 |
| 52022 | m-D1 | m-D1 | m-D1 | H | H | A-21 |
| 52023 | m-D1 | m-D1 | m-D1 | H | H | A-27 |
| 52024 | m-D1 | m-D1 | m-D1 | H | H | A-30 |
| 52025 | m-D1 | m-D1 | m-D1 | H | H | A-31 |
| 52026 | m-D1 | m-D1 | m-D1 | H | H | A-34 |
| 52027 | m-D1 | m-D1 | m-D1 | H | H | A-38 |
| 52028 | m-D1 | m-D1 | m-D1 | H | H | A-39 |
| 52029 | m-D1 | m-D1 | m-D1 | H | H | A-40 |
| 52030 | m-D1 | m-D1 | m-D1 | H | H | A-43 |
| 52031 | m-D1 | m-D1 | m-D1 | H | H | A-44 |
| 52032 | m-D1 | m-D1 | m-D1 | H | H | A-45 |
| 52033 | m-D2 | m-D2 | m-D2 | H | H | A-6 |
| 52034 | m-D2 | m-D2 | m-D2 | H | H | A-7 |
| 52035 | m-D2 | m-D2 | m-D2 | H | H | A-10 |
| 52036 | m-D2 | m-D2 | m-D2 | H | H | A-12 |
| 52037 | m-D2 | m-D2 | m-D2 | H | H | A-14 |
| 52038 | m-D2 | m-D2 | m-D2 | H | H | A-21 |
| 52039 | m-D2 | m-D2 | m-D2 | H | H | A-27 |
| 52040 | m-D2 | m-D2 | m-D2 | H | H | A-30 |
| 52041 | m-D2 | m-D2 | m-D2 | H | H | A-31 |
| 52042 | m-D2 | m-D2 | m-D2 | H | H | A-34 |
| 52043 | m-D2 | m-D2 | m-D2 | H | H | A-38 |
| 52044 | m-D2 | m-D2 | m-D2 | H | H | A-39 |
| 52045 | m-D2 | m-D2 | m-D2 | H | H | A-40 |
| 52046 | m-D2 | m-D2 | m-D2 | H | H | A-43 |
| 52047 | m-D2 | m-D2 | m-D2 | H | H | A-44 |
| 52048 | m-D2 | m-D2 | m-D2 | H | H | A-45 |
| 52049 | m-D3 | m-D3 | m-D3 | H | H | A-6 |
| 52050 | m-D3 | m-D3 | m-D3 | H | H | A-7 |
| 52051 | m-D3 | m-D3 | m-D3 | H | H | A-10 |
| 52052 | m-D3 | m-D3 | m-D3 | H | H | A-12 |
| 52053 | m-D3 | m-D3 | m-D3 | H | H | A-14 |
| 52054 | m-D3 | m-D3 | m-D3 | H | H | A-21 |
| 52055 | m-D3 | m-D3 | m-D3 | H | H | A-27 |
| 52056 | m-D3 | m-D3 | m-D3 | H | H | A-30 |
| 52057 | m-D3 | m-D3 | m-D3 | H | H | A-31 |
| 52058 | m-D3 | m-D3 | m-D3 | H | H | A-34 |
| 52059 | m-D3 | m-D3 | m-D3 | H | H | A-38 |
| 52060 | m-D3 | m-D3 | m-D3 | H | H | A-39 |
| 52061 | m-D3 | m-D3 | m-D3 | H | H | A-40 |
| 52062 | m-D3 | m-D3 | m-D3 | H | H | A-43 |
| 52063 | m-D3 | m-D3 | m-D3 | H | H | A-44 |
| 52064 | m-D3 | m-D3 | m-D3 | H | H | A-45 |
| 52065 | m-D4 | m-D4 | m-D4 | H | H | A-6 |
| 52066 | m-D4 | m-D4 | m-D4 | H | H | A-7 |
| 52067 | m-D4 | m-D4 | m-D4 | H | H | A-10 |
| 52068 | m-D4 | m-D4 | m-D4 | H | H | A-12 |
| 52069 | m-D4 | m-D4 | m-D4 | H | H | A-14 |
| 52070 | m-D4 | m-D4 | m-D4 | H | H | A-21 |
| 52071 | m-D4 | m-D4 | m-D4 | H | H | A-27 |
| 52072 | m-D4 | m-D4 | m-D4 | H | H | A-30 |
| 52073 | m-D4 | m-D4 | m-D4 | H | H | A-31 |
| 52074 | m-D4 | m-D4 | m-D4 | H | H | A-34 |
| 52075 | m-D4 | m-D4 | m-D4 | H | H | A-38 |
| 52076 | m-D4 | m-D4 | m-D4 | H | H | A-39 |
| 52077 | m-D4 | m-D4 | m-D4 | H | H | A-40 |
| 52078 | m-D4 | m-D4 | m-D4 | H | H | A-43 |
| 52079 | m-D4 | m-D4 | m-D4 | H | H | A-44 |
| 52080 | m-D4 | m-D4 | m-D4 | H | H | A-45 |
| 52081 | m-D5 | m-D5 | m-D5 | H | H | A-6 |
| 52082 | m-D5 | m-D5 | m-D5 | H | H | A-7 |
| 52083 | m-D5 | m-D5 | m-D5 | H | H | A-10 |
| 52084 | m-D5 | m-D5 | m-D5 | H | H | A-12 |
| 52085 | m-D5 | m-D5 | m-D5 | H | H | A-14 |
| 52086 | m-D5 | m-D5 | m-D5 | H | H | A-21 |
| 52087 | m-D5 | m-D5 | m-D5 | H | H | A-27 |
| 52088 | m-D5 | m-D5 | m-D5 | H | H | A-30 |
| 52089 | m-D5 | m-D5 | m-D5 | H | H | A-31 |
| 52090 | m-D5 | m-D5 | m-D5 | H | H | A-34 |
| 52091 | m-D5 | m-D5 | m-D5 | H | H | A-38 |
| 52092 | m-D5 | m-D5 | m-D5 | H | H | A-39 |
| 52093 | m-D5 | m-D5 | m-D5 | H | H | A-40 |
| 52094 | m-D5 | m-D5 | m-D5 | H | H | A-43 |
| 52095 | m-D5 | m-D5 | m-D5 | H | H | A-44 |
| 52096 | m-D5 | m-D5 | m-D5 | H | H | A-45 |
| 52097 | m-D6 | m-D6 | m-D6 | H | H | A-6 |
| 52098 | m-D6 | m-D6 | m-D6 | H | H | A-7 |
| 52099 | m-D6 | m-D6 | m-D6 | H | H | A-10 |
| 52100 | m-D6 | m-D6 | m-D6 | H | H | A-12 |
| 52101 | m-D6 | m-D6 | m-D6 | H | H | A-14 |
| 52102 | m-D6 | m-D6 | m-D6 | H | H | A-21 |
| 52103 | m-D6 | m-D6 | m-D6 | H | H | A-27 |
| 52104 | m-D6 | m-D6 | m-D6 | H | H | A-30 |
| 52105 | m-D6 | m-D6 | m-D6 | H | H | A-31 |
| 52106 | m-D6 | m-D6 | m-D6 | H | H | A-34 |
| 52107 | m-D6 | m-D6 | m-D6 | H | H | A-38 |
| 52108 | m-D6 | m-D6 | m-D6 | H | H | A-39 |
| 52109 | m-D6 | m-D6 | m-D6 | H | H | A-40 |
| 52110 | m-D6 | m-D6 | m-D6 | H | H | A-43 |
| 52111 | m-D6 | m-D6 | m-D6 | H | H | A-44 |
| 52112 | m-D6 | m-D6 | m-D6 | H | H | A-45 |
| 52113 | m-D7 | m-D7 | m-D7 | H | H | A-6 |
| 52114 | m-D7 | m-D7 | m-D7 | H | H | A-7 |
| 52115 | m-D7 | m-D7 | m-D7 | H | H | A-10 |
| 52116 | m-D7 | m-D7 | m-D7 | H | H | A-12 |
| 52117 | m-D7 | m-D7 | m-D7 | H | H | A-14 |
| 52118 | m-D7 | m-D7 | m-D7 | H | H | A-21 |
| 52119 | m-D7 | m-D7 | m-D7 | H | H | A-27 |
| 52120 | m-D7 | m-D7 | m-D7 | H | H | A-30 |
| 52121 | m-D7 | m-D7 | m-D7 | H | H | A-31 |
| 52122 | m-D7 | m-D7 | m-D7 | H | H | A-34 |
| 52123 | m-D7 | m-D7 | m-D7 | H | H | A-38 |
| 52124 | m-D7 | m-D7 | m-D7 | H | H | A-39 |
| 52125 | m-D7 | m-D7 | m-D7 | H | H | A-40 |
| 52126 | m-D7 | m-D7 | m-D7 | H | H | A-43 |
| 52127 | m-D7 | m-D7 | m-D7 | H | H | A-44 |
| 52128 | m-D7 | m-D7 | m-D7 | H | H | A-45 |
| 52129 | m-D8 | m-D8 | m-D8 | H | H | A-6 |
| 52130 | m-D8 | m-D8 | m-D8 | H | H | A-7 |
| 52131 | m-D8 | m-D8 | m-D8 | H | H | A-10 |
| 52132 | m-D8 | m-D8 | m-D8 | H | H | A-12 |
| 52133 | m-D8 | m-D8 | m-D8 | H | H | A-14 |
| 52134 | m-D8 | m-D8 | m-D8 | H | H | A-21 |
| 52135 | m-D8 | m-D8 | m-D8 | H | H | A-27 |
| 52136 | m-D8 | m-D8 | m-D8 | H | H | A-30 |
| 52137 | m-D8 | m-D8 | m-D8 | H | H | A-31 |
| 52138 | m-D8 | m-D8 | m-D8 | H | H | A-34 |
| 52139 | m-D8 | m-D8 | m-D8 | H | H | A-38 |
| 52140 | m-D8 | m-D8 | m-D8 | H | H | A-39 |
| 52141 | m-D8 | m-D8 | m-D8 | H | H | A-40 |
| 52142 | m-D8 | m-D8 | m-D8 | H | H | A-43 |
| 52143 | m-D8 | m-D8 | m-D8 | H | H | A-44 |
| 52144 | m-D8 | m-D8 | m-D8 | H | H | A-45 |
| 52145 | m-D9 | m-D9 | m-D9 | H | H | A-6 |
| 52146 | m-D9 | m-D9 | m-D9 | H | H | A-7 |
| 52147 | m-D9 | m-D9 | m-D9 | H | H | A-10 |
| 52148 | m-D9 | m-D9 | m-D9 | H | H | A-12 |
| 52149 | m-D9 | m-D9 | m-D9 | H | H | A-14 |
| 52150 | m-D9 | m-D9 | m-D9 | H | H | A-21 |
| 52151 | m-D9 | m-D9 | m-D9 | H | H | A-27 |
| 52152 | m-D9 | m-D9 | m-D9 | H | H | A-30 |
| 52153 | m-D9 | m-D9 | m-D9 | H | H | A-31 |
| 52154 | m-D9 | m-D9 | m-D9 | H | H | A-34 |
| 52155 | m-D9 | m-D9 | m-D9 | H | H | A-38 |
| 52156 | m-D9 | m-D9 | m-D9 | H | H | A-39 |
| 52157 | m-D9 | m-D9 | m-D9 | H | H | A-40 |
| 52158 | m-D9 | m-D9 | m-D9 | H | H | A-43 |
| 52159 | m-D9 | m-D9 | m-D9 | H | H | A-44 |
| 52160 | m-D9 | m-D9 | m-D9 | H | H | A-45 |
| 52161 | m-D10 | m-D10 | m-D10 | H | H | A-6 |
| 52162 | m-D10 | m-D10 | m-D10 | H | H | A-7 |
| 52163 | m-D10 | m-D10 | m-D10 | H | H | A-10 |
| 52164 | m-D10 | m-D10 | m-D10 | H | H | A-12 |
| 52165 | m-D10 | m-D10 | m-D10 | H | H | A-14 |
| 52166 | m-D10 | m-D10 | m-D10 | H | H | A-21 |
| 52167 | m-D10 | m-D10 | m-D10 | H | H | A-27 |
| 52168 | m-D10 | m-D10 | m-D10 | H | H | A-30 |
| 52169 | m-D10 | m-D10 | m-D10 | H | H | A-31 |
| 52170 | m-D10 | m-D10 | m-D10 | H | H | A-34 |
| 52171 | m-D10 | m-D10 | m-D10 | H | H | A-38 |
| 52172 | m-D10 | m-D10 | m-D10 | H | H | A-39 |
| 52173 | m-D10 | m-D10 | m-D10 | H | H | A-40 |
| 52174 | m-D10 | m-D10 | m-D10 | H | H | A-43 |
| 52175 | m-D10 | m-D10 | m-D10 | H | H | A-44 |
| 52176 | m-D10 | m-D10 | m-D10 | H | H | A-45 |
| 52177 | m-D11 | m-D11 | m-D11 | H | H | A-6 |
| 52178 | m-D11 | m-D11 | m-D11 | H | H | A-7 |
| 52179 | m-D11 | m-D11 | m-D11 | H | H | A-10 |
| 52180 | m-D11 | m-D11 | m-D11 | H | H | A-12 |
| 52181 | m-D11 | m-D11 | m-D11 | H | H | A-14 |
| 52182 | m-D11 | m-D11 | m-D11 | H | H | A-21 |
| 52183 | m-D11 | m-D11 | m-D11 | H | H | A-27 |
| 52184 | m-D11 | m-D11 | m-D11 | H | H | A-30 |
| 52185 | m-D11 | m-D11 | m-D11 | H | H | A-31 |
| 52186 | m-D11 | m-D11 | m-D11 | H | H | A-34 |
| 52187 | m-D11 | m-D11 | m-D11 | H | H | A-38 |
| 52188 | m-D11 | m-D11 | m-D11 | H | H | A-39 |
| 52189 | m-D11 | m-D11 | m-D11 | H | H | A-40 |
| 52190 | m-D11 | m-D11 | m-D11 | H | H | A-43 |
| 52191 | m-D11 | m-D11 | m-D11 | H | H | A-44 |
| 52192 | m-D11 | m-D11 | m-D11 | H | H | A-45 |
| 52193 | m-D12 | m-D12 | m-D12 | H | H | A-6 |
| 52194 | m-D12 | m-D12 | m-D12 | H | H | A-7 |
| 52195 | m-D12 | m-D12 | m-D12 | H | H | A-10 |
| 52196 | m-D12 | m-D12 | m-D12 | H | H | A-12 |
| 52197 | m-D12 | m-D12 | m-D12 | H | H | A-14 |
| 52198 | m-D12 | m-D12 | m-D12 | H | H | A-21 |
| 52199 | m-D12 | m-D12 | m-D12 | H | H | A-27 |
| 52200 | m-D12 | m-D12 | m-D12 | H | H | A-30 |
| 52201 | m-D12 | m-D12 | m-D12 | H | H | A-31 |
| 52202 | m-D12 | m-D12 | m-D12 | H | H | A-34 |
| 52203 | m-D12 | m-D12 | m-D12 | H | H | A-38 |
| 52204 | m-D12 | m-D12 | m-D12 | H | H | A-39 |
| 52205 | m-D12 | m-D12 | m-D12 | H | H | A-40 |
| 52206 | m-D12 | m-D12 | m-D12 | H | H | A-43 |
| 52207 | m-D12 | m-D12 | m-D12 | H | H | A-44 |
| 52208 | m-D12 | m-D12 | m-D12 | H | H | A-45 |
| 52209 | m-D13 | m-D13 | m-D13 | H | H | A-6 |
| 52210 | m-D13 | m-D13 | m-D13 | H | H | A-7 |
| 52211 | m-D13 | m-D13 | m-D13 | H | H | A-10 |
| 52212 | m-D13 | m-D13 | m-D13 | H | H | A-12 |
| 52213 | m-D13 | m-D13 | m-D13 | H | H | A-14 |
| 52214 | m-D13 | m-D13 | m-D13 | H | H | A-21 |
| 52215 | m-D13 | m-D13 | m-D13 | H | H | A-27 |
| 52216 | m-D13 | m-D13 | m-D13 | H | H | A-30 |
| 52217 | m-D13 | m-D13 | m-D13 | H | H | A-31 |
| 52218 | m-D13 | m-D13 | m-D13 | H | H | A-34 |
| 52219 | m-D13 | m-D13 | m-D13 | H | H | A-38 |
| 52220 | m-D13 | m-D13 | m-D13 | H | H | A-39 |
| 52221 | m-D13 | m-D13 | m-D13 | H | H | A-40 |
| 52222 | m-D13 | m-D13 | m-D13 | H | H | A-43 |
| 52223 | m-D13 | m-D13 | m-D13 | H | H | A-44 |
| 52224 | m-D13 | m-D13 | m-D13 | H | H | A-45 |
| 52225 | m-D14 | m-D14 | m-D14 | H | H | A-6 |
| 52226 | m-D14 | m-D14 | m-D14 | H | H | A-7 |
| 52227 | m-D14 | m-D14 | m-D14 | H | H | A-10 |
| 52228 | m-D14 | m-D14 | m-D14 | H | H | A-12 |
| 52229 | m-D14 | m-D14 | m-D14 | H | H | A-14 |
| 52230 | m-D14 | m-D14 | m-D14 | H | H | A-21 |
| 52231 | m-D14 | m-D14 | m-D14 | H | H | A-27 |
| 52232 | m-D14 | m-D14 | m-D14 | H | H | A-30 |
| 52233 | m-D14 | m-D14 | m-D14 | H | H | A-31 |
| 52234 | m-D14 | m-D14 | m-D14 | H | H | A-34 |
| 52235 | m-D14 | m-D14 | m-D14 | H | H | A-38 |
| 52236 | m-D14 | m-D14 | m-D14 | H | H | A-39 |
| 52237 | m-D14 | m-D14 | m-D14 | H | H | A-40 |
| 52238 | m-D14 | m-D14 | m-D14 | H | H | A-43 |
| 52239 | m-D14 | m-D14 | m-D14 | H | H | A-44 |
| 52240 | m-D14 | m-D14 | m-D14 | H | H | A-45 |
| 52241 | m-D1 | m-D1 | H | m-D1 | H | A-6 |
| 52242 | m-D1 | m-D1 | H | m-D1 | H | A-7 |
| 52243 | m-D1 | m-D1 | H | m-D1 | H | A-10 |
| 52244 | m-D1 | m-D1 | H | m-D1 | H | A-12 |
| 52245 | m-D1 | m-D1 | H | m-D1 | H | A-14 |
| 52246 | m-D1 | m-D1 | H | m-D1 | H | A-21 |
| 52247 | m-D1 | m-D1 | H | m-D1 | H | A-27 |
| 52248 | m-D1 | m-D1 | H | m-D1 | H | A-30 |
| 52249 | m-D1 | m-D1 | H | m-D1 | H | A-31 |
| 52250 | m-D1 | m-D1 | H | m-D1 | H | A-34 |
| 52251 | m-D1 | m-D1 | H | m-D1 | H | A-38 |
| 52252 | m-D1 | m-D1 | H | m-D1 | H | A-39 |
| 52253 | m-D1 | m-D1 | H | m-D1 | H | A-40 |
| 52254 | m-D1 | m-D1 | H | m-D1 | H | A-43 |
| 52255 | m-D1 | m-D1 | H | m-D1 | H | A-44 |
| 52256 | m-D1 | m-D1 | H | m-D1 | H | A-45 |
| 52257 | m-D2 | m-D2 | H | m-D2 | H | A-6 |
| 52258 | m-D2 | m-D2 | H | m-D2 | H | A-7 |
| 52259 | m-D2 | m-D2 | H | m-D2 | H | A-10 |
| 52260 | m-D2 | m-D2 | H | m-D2 | H | A-12 |
| 52261 | m-D2 | m-D2 | H | m-D2 | H | A-14 |
| 52262 | m-D2 | m-D2 | H | m-D2 | H | A-21 |
| 52263 | m-D2 | m-D2 | H | m-D2 | H | A-27 |
| 52264 | m-D2 | m-D2 | H | m-D2 | H | A-30 |
| 52265 | m-D2 | m-D2 | H | m-D2 | H | A-31 |
| 52266 | m-D2 | m-D2 | H | m-D2 | H | A-34 |
| 52267 | m-D2 | m-D2 | H | m-D2 | H | A-38 |
| 52268 | m-D2 | m-D2 | H | m-D2 | H | A-39 |
| 52269 | m-D2 | m-D2 | H | m-D2 | H | A-40 |
| 52270 | m-D2 | m-D2 | H | m-D2 | H | A-43 |
| 52271 | m-D2 | m-D2 | H | m-D2 | H | A-44 |
| 52272 | m-D2 | m-D2 | H | m-D2 | H | A-45 |
| 52273 | m-D3 | m-D3 | H | m-D3 | H | A-6 |
| 52274 | m-D3 | m-D3 | H | m-D3 | H | A-7 |
| 52275 | m-D3 | m-D3 | H | m-D3 | H | A-10 |
| 52276 | m-D3 | m-D3 | H | m-D3 | H | A-12 |
| 52277 | m-D3 | m-D3 | H | m-D3 | H | A-14 |
| 52278 | m-D3 | m-D3 | H | m-D3 | H | A-21 |
| 52279 | m-D3 | m-D3 | H | m-D3 | H | A-27 |
| 52280 | m-D3 | m-D3 | H | m-D3 | H | A-30 |
| 52281 | m-D3 | m-D3 | H | m-D3 | H | A-31 |
| 52282 | m-D3 | m-D3 | H | m-D3 | H | A-34 |
| 52283 | m-D3 | m-D3 | H | m-D3 | H | A-38 |
| 52284 | m-D3 | m-D3 | H | m-D3 | H | A-39 |
| 52285 | m-D3 | m-D3 | H | m-D3 | H | A-40 |
| 52286 | m-D3 | m-D3 | H | m-D3 | H | A-43 |
| 52287 | m-D3 | m-D3 | H | m-D3 | H | A-44 |
| 52288 | m-D3 | m-D3 | H | m-D3 | H | A-45 |
| 52289 | m-D4 | m-D4 | H | m-D4 | H | A-6 |
| 52290 | m-D4 | m-D4 | H | m-D4 | H | A-7 |
| 52291 | m-D4 | m-D4 | H | m-D4 | H | A-10 |
| 52292 | m-D4 | m-D4 | H | m-D4 | H | A-12 |
| 52293 | m-D4 | m-D4 | H | m-D4 | H | A-14 |
| 52294 | m-D4 | m-D4 | H | m-D4 | H | A-21 |
| 52295 | m-D4 | m-D4 | H | m-D4 | H | A-27 |
| 52296 | m-D4 | m-D4 | H | m-D4 | H | A-30 |
| 52297 | m-D4 | m-D4 | H | m-D4 | H | A-31 |
| 52298 | m-D4 | m-D4 | H | m-D4 | H | A-34 |
| 52299 | m-D4 | m-D4 | H | m-D4 | H | A-38 |
| 52300 | m-D4 | m-D4 | H | m-D4 | H | A-39 |
| 52301 | m-D4 | m-D4 | H | m-D4 | H | A-40 |
| 52302 | m-D4 | m-D4 | H | m-D4 | H | A-43 |
| 52303 | m-D4 | m-D4 | H | m-D4 | H | A-44 |
| 52304 | m-D4 | m-D4 | H | m-D4 | H | A-45 |
| 52305 | m-D5 | m-D5 | H | m-D5 | H | A-6 |
| 52306 | m-D5 | m-D5 | H | m-D5 | H | A-7 |
| 52307 | m-D5 | m-D5 | H | m-D5 | H | A-10 |
| 52308 | m-D5 | m-D5 | H | m-D5 | H | A-12 |
| 52309 | m-D5 | m-D5 | H | m-D5 | H | A-14 |
| 52310 | m-D5 | m-D5 | H | m-D5 | H | A-21 |
| 52311 | m-D5 | m-D5 | H | m-D5 | H | A-27 |
| 52312 | m-D5 | m-D5 | H | m-D5 | H | A-30 |
| 52313 | m-D5 | m-D5 | H | m-D5 | H | A-31 |
| 52314 | m-D5 | m-D5 | H | m-D5 | H | A-34 |
| 52315 | m-D5 | m-D5 | H | m-D5 | H | A-38 |
| 52316 | m-D5 | m-D5 | H | m-D5 | H | A-39 |
| 52317 | m-D5 | m-D5 | H | m-D5 | H | A-40 |
| 52318 | m-D5 | m-D5 | H | m-D5 | H | A-43 |
| 52319 | m-D5 | m-D5 | H | m-D5 | H | A-44 |
| 52320 | m-D5 | m-D5 | H | m-D5 | H | A-45 |
| 52321 | m-D6 | m-D6 | H | m-D6 | H | A-6 |
| 52322 | m-D6 | m-D6 | H | m-D6 | H | A-7 |
| 52323 | m-D6 | m-D6 | H | m-D6 | H | A-10 |
| 52324 | m-D6 | m-D6 | H | m-D6 | H | A-12 |
| 52325 | m-D6 | m-D6 | H | m-D6 | H | A-14 |
| 52326 | m-D6 | m-D6 | H | m-D6 | H | A-21 |
| 52327 | m-D6 | m-D6 | H | m-D6 | H | A-27 |
| 52328 | m-D6 | m-D6 | H | m-D6 | H | A-30 |
| 52329 | m-D6 | m-D6 | H | m-D6 | H | A-31 |
| 52330 | m-D6 | m-D6 | H | m-D6 | H | A-34 |
| 52331 | m-D6 | m-D6 | H | m-D6 | H | A-38 |
| 52332 | m-D6 | m-D6 | H | m-D6 | H | A-39 |
| 52333 | m-D6 | m-D6 | H | m-D6 | H | A-40 |
| 52334 | m-D6 | m-D6 | H | m-D6 | H | A-43 |
| 52335 | m-D6 | m-D6 | H | m-D6 | H | A-44 |
| 52336 | m-D6 | m-D6 | H | m-D6 | H | A-45 |
| 52337 | m-D7 | m-D7 | H | m-D7 | H | A-6 |
| 52338 | m-D7 | m-D7 | H | m-D7 | H | A-7 |
| 52339 | m-D7 | m-D7 | H | m-D7 | H | A-10 |
| 52340 | m-D7 | m-D7 | H | m-D7 | H | A-12 |
| 52341 | m-D7 | m-D7 | H | m-D7 | H | A-14 |
| 52342 | m-D7 | m-D7 | H | m-D7 | H | A-21 |
| 52343 | m-D7 | m-D7 | H | m-D7 | H | A-27 |
| 52344 | m-D7 | m-D7 | H | m-D7 | H | A-30 |
| 52345 | m-D7 | m-D7 | H | m-D7 | H | A-31 |
| 52346 | m-D7 | m-D7 | H | m-D7 | H | A-34 |
| 52347 | m-D7 | m-D7 | H | m-D7 | H | A-38 |
| 52348 | m-D7 | m-D7 | H | m-D7 | H | A-39 |
| 52349 | m-D7 | m-D7 | H | m-D7 | H | A-40 |
| 52350 | m-D7 | m-D7 | H | m-D7 | H | A-43 |
| 52351 | m-D7 | m-D7 | H | m-D7 | H | A-44 |
| 52352 | m-D7 | m-D7 | H | m-D7 | H | A-45 |
| 52353 | m-D8 | m-D8 | H | m-D8 | H | A-6 |
| 52354 | m-D8 | m-D8 | H | m-D8 | H | A-7 |
| 52355 | m-D8 | m-D8 | H | m-D8 | H | A-10 |
| 52356 | m-D8 | m-D8 | H | m-D8 | H | A-12 |
| 52357 | m-D8 | m-D8 | H | m-D8 | H | A-14 |
| 52358 | m-D8 | m-D8 | H | m-D8 | H | A-21 |
| 52359 | m-D8 | m-D8 | H | m-D8 | H | A-27 |
| 52360 | m-D8 | m-D8 | H | m-D8 | H | A-30 |
| 52361 | m-D8 | m-D8 | H | m-D8 | H | A-31 |
| 52362 | m-D8 | m-D8 | H | m-D8 | H | A-34 |
| 52363 | m-D8 | m-D8 | H | m-D8 | H | A-38 |
| 52364 | m-D8 | m-D8 | H | m-D8 | H | A-39 |
| 52365 | m-D8 | m-D8 | H | m-D8 | H | A-40 |
| 52366 | m-D8 | m-D8 | H | m-D8 | H | A-43 |
| 52367 | m-D8 | m-D8 | H | m-D8 | H | A-44 |
| 52368 | m-D8 | m-D8 | H | m-D8 | H | A-45 |
| 52369 | m-D9 | m-D9 | H | m-D9 | H | A-6 |
| 52370 | m-D9 | m-D9 | H | m-D9 | H | A-7 |
| 52371 | m-D9 | m-D9 | H | m-D9 | H | A-10 |
| 52372 | m-D9 | m-D9 | H | m-D9 | H | A-12 |
| 52373 | m-D9 | m-D9 | H | m-D9 | H | A-14 |
| 52374 | m-D9 | m-D9 | H | m-D9 | H | A-21 |
| 52375 | m-D9 | m-D9 | H | m-D9 | H | A-27 |
| 52376 | m-D9 | m-D9 | H | m-D9 | H | A-30 |
| 52377 | m-D9 | m-D9 | H | m-D9 | H | A-31 |
| 52378 | m-D9 | m-D9 | H | m-D9 | H | A-34 |
| 52379 | m-D9 | m-D9 | H | m-D9 | H | A-38 |
| 52380 | m-D9 | m-D9 | H | m-D9 | H | A-39 |
| 52381 | m-D9 | m-D9 | H | m-D9 | H | A-40 |
| 52382 | m-D9 | m-D9 | H | m-D9 | H | A-43 |
| 52383 | m-D9 | m-D9 | H | m-D9 | H | A-44 |
| 52384 | m-D9 | m-D9 | H | m-D9 | H | A-45 |
| 52385 | m-D10 | m-D10 | H | m-D10 | H | A-6 |
| 52386 | m-D10 | m-D10 | H | m-D10 | H | A-7 |
| 52387 | m-D10 | m-D10 | H | m-D10 | H | A-10 |
| 52388 | m-D10 | m-D10 | H | m-D10 | H | A-12 |
| 52389 | m-D10 | m-D10 | H | m-D10 | H | A-14 |
| 52390 | m-D10 | m-D10 | H | m-D10 | H | A-21 |
| 52391 | m-D10 | m-D10 | H | m-D10 | H | A-27 |
| 52392 | m-D10 | m-D10 | H | m-D10 | H | A-30 |
| 52393 | m-D10 | m-D10 | H | m-D10 | H | A-31 |
| 52394 | m-D10 | m-D10 | H | m-D10 | H | A-34 |
| 52395 | m-D10 | m-D10 | H | m-D10 | H | A-38 |
| 52396 | m-D10 | m-D10 | H | m-D10 | H | A-39 |
| 52397 | m-D10 | m-D10 | H | m-D10 | H | A-40 |
| 52398 | m-D10 | m-D10 | H | m-D10 | H | A-43 |
| 52399 | m-D10 | m-D10 | H | m-D10 | H | A-44 |
| 52400 | m-D10 | m-D10 | H | m-D10 | H | A-45 |
| 52401 | m-D11 | m-D11 | H | m-D11 | H | A-6 |
| 52402 | m-D11 | m-D11 | H | m-D11 | H | A-7 |
| 52403 | m-D11 | m-D11 | H | m-D11 | H | A-10 |
| 52404 | m-D11 | m-D11 | H | m-D11 | H | A-12 |
| 52405 | m-D11 | m-D11 | H | m-D11 | H | A-14 |
| 52406 | m-D11 | m-D11 | H | m-D11 | H | A-21 |
| 52407 | m-D11 | m-D11 | H | m-D11 | H | A-27 |
| 52408 | m-D11 | m-D11 | H | m-D11 | H | A-30 |
| 52409 | m-D11 | m-D11 | H | m-D11 | H | A-31 |
| 52410 | m-D11 | m-D11 | H | m-D11 | H | A-34 |
| 52411 | m-D11 | m-D11 | H | m-D11 | H | A-38 |
| 52412 | m-D11 | m-D11 | H | m-D11 | H | A-39 |
| 52413 | m-D11 | m-D11 | H | m-D11 | H | A-40 |
| 52414 | m-D11 | m-D11 | H | m-D11 | H | A-43 |
| 52415 | m-D11 | m-D11 | H | m-D11 | H | A-44 |
| 52416 | m-D11 | m-D11 | H | m-D11 | H | A-45 |
| 52417 | m-D12 | m-D12 | H | m-D12 | H | A-6 |
| 52418 | m-D12 | m-D12 | H | m-D12 | H | A-7 |
| 52419 | m-D12 | m-D12 | H | m-D12 | H | A-10 |
| 52420 | m-D12 | m-D12 | H | m-D12 | H | A-12 |
| 52421 | m-D12 | m-D12 | H | m-D12 | H | A-14 |
| 52422 | m-D12 | m-D12 | H | m-D12 | H | A-21 |
| 52423 | m-D12 | m-D12 | H | m-D12 | H | A-27 |
| 52424 | m-D12 | m-D12 | H | m-D12 | H | A-30 |
| 52425 | m-D12 | m-D12 | H | m-D12 | H | A-31 |
| 52426 | m-D12 | m-D12 | H | m-D12 | H | A-34 |
| 52427 | m-D12 | m-D12 | H | m-D12 | H | A-38 |
| 52428 | m-D12 | m-D12 | H | m-D12 | H | A-39 |
| 52429 | m-D12 | m-D12 | H | m-D12 | H | A-40 |
| 52430 | m-D12 | m-D12 | H | m-D12 | H | A-43 |
| 52431 | m-D12 | m-D12 | H | m-D12 | H | A-44 |
| 52432 | m-D12 | m-D12 | H | m-D12 | H | A-45 |
| 52433 | m-D13 | m-D13 | H | m-D13 | H | A-6 |
| 52434 | m-D13 | m-D13 | H | m-D13 | H | A-7 |
| 52435 | m-D13 | m-D13 | H | m-D13 | H | A-10 |
| 52436 | m-D13 | m-D13 | H | m-D13 | H | A-12 |
| 52437 | m-D13 | m-D13 | H | m-D13 | H | A-14 |
| 52438 | m-D13 | m-D13 | H | m-D13 | H | A-21 |
| 52439 | m-D13 | m-D13 | H | m-D13 | H | A-27 |
| 52440 | m-D13 | m-D13 | H | m-D13 | H | A-30 |
| 52441 | m-D13 | m-D13 | H | m-D13 | H | A-31 |
| 52442 | m-D13 | m-D13 | H | m-D13 | H | A-34 |
| 52443 | m-D13 | m-D13 | H | m-D13 | H | A-38 |
| 52444 | m-D13 | m-D13 | H | m-D13 | H | A-39 |
| 52445 | m-D13 | m-D13 | H | m-D13 | H | A-40 |
| 52446 | m-D13 | m-D13 | H | m-D13 | H | A-43 |
| 52447 | m-D13 | m-D13 | H | m-D13 | H | A-44 |
| 52448 | m-D13 | m-D13 | H | m-D13 | H | A-45 |
| 52449 | m-D14 | m-D14 | H | m-D14 | H | A-6 |
| 52450 | m-D14 | m-D14 | H | m-D14 | H | A-7 |
| 52451 | m-D14 | m-D14 | H | m-D14 | H | A-10 |
| 52452 | m-D14 | m-D14 | H | m-D14 | H | A-12 |
| 52453 | m-D14 | m-D14 | H | m-D14 | H | A-14 |
| 52454 | m-D14 | m-D14 | H | m-D14 | H | A-21 |
| 52455 | m-D14 | m-D14 | H | m-D14 | H | A-27 |
| 52456 | m-D14 | m-D14 | H | m-D14 | H | A-30 |
| 52457 | m-D14 | m-D14 | H | m-D14 | H | A-31 |
| 52458 | m-D14 | m-D14 | H | m-D14 | H | A-34 |
| 52459 | m-D14 | m-D14 | H | m-D14 | H | A-38 |
| 52460 | m-D14 | m-D14 | H | m-D14 | H | A-39 |
| 52461 | m-D14 | m-D14 | H | m-D14 | H | A-40 |
| 52462 | m-D14 | m-D14 | H | m-D14 | H | A-43 |
| 52463 | m-D14 | m-D14 | H | m-D14 | H | A-44 |
| 52464 | m-D14 | m-D14 | H | m-D14 | H | A-45 |
| 52465 | m-D1 | H | m-D1 | m-D1 | H | A-6 |
| 52466 | m-D1 | H | m-D1 | m-D1 | H | A-7 |
| 52467 | m-D1 | H | m-D1 | m-D1 | H | A-10 |
| 52468 | m-D1 | H | m-D1 | m-D1 | H | A-12 |
| 52469 | m-D1 | H | m-D1 | m-D1 | H | A-14 |
| 52470 | m-D1 | H | m-D1 | m-D1 | H | A-21 |
| 52471 | m-D1 | H | m-D1 | m-D1 | H | A-27 |
| 52472 | m-D1 | H | m-D1 | m-D1 | H | A-30 |
| 52473 | m-D1 | H | m-D1 | m-D1 | H | A-31 |
| 52474 | m-D1 | H | m-D1 | m-D1 | H | A-34 |
| 52475 | m-D1 | H | m-D1 | m-D1 | H | A-38 |
| 52476 | m-D1 | H | m-D1 | m-D1 | H | A-39 |
| 52477 | m-D1 | H | m-D1 | m-D1 | H | A-40 |
| 52478 | m-D1 | H | m-D1 | m-D1 | H | A-43 |
| 52479 | m-D1 | H | m-D1 | m-D1 | H | A-44 |
| 52480 | m-D1 | H | m-D1 | m-D1 | H | A-45 |
| 52481 | m-D2 | H | m-D2 | m-D2 | H | A-6 |
| 52482 | m-D2 | H | m-D2 | m-D2 | H | A-7 |
| 52483 | m-D2 | H | m-D2 | m-D2 | H | A-10 |
| 52484 | m-D2 | H | m-D2 | m-D2 | H | A-12 |
| 52485 | m-D2 | H | m-D2 | m-D2 | H | A-14 |
| 52486 | m-D2 | H | m-D2 | m-D2 | H | A-21 |
| 52487 | m-D2 | H | m-D2 | m-D2 | H | A-27 |
| 52488 | m-D2 | H | m-D2 | m-D2 | H | A-30 |
| 52489 | m-D2 | H | m-D2 | m-D2 | H | A-31 |
| 52490 | m-D2 | H | m-D2 | m-D2 | H | A-34 |
| 52491 | m-D2 | H | m-D2 | m-D2 | H | A-38 |
| 52492 | m-D2 | H | m-D2 | m-D2 | H | A-39 |
| 52493 | m-D2 | H | m-D2 | m-D2 | H | A-40 |
| 52494 | m-D2 | H | m-D2 | m-D2 | H | A-43 |
| 52495 | m-D2 | H | m-D2 | m-D2 | H | A-44 |
| 52496 | m-D2 | H | m-D2 | m-D2 | H | A-45 |
| 52497 | m-D3 | H | m-D3 | m-D3 | H | A-6 |
| 52498 | m-D3 | H | m-D3 | m-D3 | H | A-7 |
| 52499 | m-D3 | H | m-D3 | m-D3 | H | A-10 |
| 52500 | m-D3 | H | m-D3 | m-D3 | H | A-12 |
| 52501 | m-D3 | H | m-D3 | m-D3 | H | A-14 |
| 52502 | m-D3 | H | m-D3 | m-D3 | H | A-21 |
| 52503 | m-D3 | H | m-D3 | m-D3 | H | A-27 |
| 52504 | m-D3 | H | m-D3 | m-D3 | H | A-30 |
| 52505 | m-D3 | H | m-D3 | m-D3 | H | A-31 |
| 52506 | m-D3 | H | m-D3 | m-D3 | H | A-34 |
| 52507 | m-D3 | H | m-D3 | m-D3 | H | A-38 |
| 52508 | m-D3 | H | m-D3 | m-D3 | H | A-39 |
| 52509 | m-D3 | H | m-D3 | m-D3 | H | A-40 |
| 52510 | m-D3 | H | m-D3 | m-D3 | H | A-43 |
| 52511 | m-D3 | H | m-D3 | m-D3 | H | A-44 |
| 52512 | m-D3 | H | m-D3 | m-D3 | H | A-45 |
| 52513 | m-D4 | H | m-D4 | m-D4 | H | A-6 |
| 52514 | m-D4 | H | m-D4 | m-D4 | H | A-7 |
| 52515 | m-D4 | H | m-D4 | m-D4 | H | A-10 |
| 52516 | m-D4 | H | m-D4 | m-D4 | H | A-12 |
| 52517 | m-D4 | H | m-D4 | m-D4 | H | A-14 |
| 52518 | m-D4 | H | m-D4 | m-D4 | H | A-21 |
| 52519 | m-D4 | H | m-D4 | m-D4 | H | A-27 |
| 52520 | m-D4 | H | m-D4 | m-D4 | H | A-30 |
| 52521 | m-D4 | H | m-D4 | m-D4 | H | A-31 |
| 52522 | m-D4 | H | m-D4 | m-D4 | H | A-34 |
| 52523 | m-D4 | H | m-D4 | m-D4 | H | A-38 |
| 52524 | m-D4 | H | m-D4 | m-D4 | H | A-39 |
| 52525 | m-D4 | H | m-D4 | m-D4 | H | A-40 |
| 52526 | m-D4 | H | m-D4 | m-D4 | H | A-43 |
| 52527 | m-D4 | H | m-D4 | m-D4 | H | A-44 |
| 52528 | m-D4 | H | m-D4 | m-D4 | H | A-45 |
| 52529 | m-D5 | H | m-D5 | m-D5 | H | A-6 |
| 52530 | m-D5 | H | m-D5 | m-D5 | H | A-7 |
| 52531 | m-D5 | H | m-D5 | m-D5 | H | A-10 |
| 52532 | m-D5 | H | m-D5 | m-D5 | H | A-12 |
| 52533 | m-D5 | H | m-D5 | m-D5 | H | A-14 |
| 52534 | m-D5 | H | m-D5 | m-D5 | H | A-21 |
| 52535 | m-D5 | H | m-D5 | m-D5 | H | A-27 |
| 52536 | m-D5 | H | m-D5 | m-D5 | H | A-30 |
| 52537 | m-D5 | H | m-D5 | m-D5 | H | A-31 |
| 52538 | m-D5 | H | m-D5 | m-D5 | H | A-34 |
| 52539 | m-D5 | H | m-D5 | m-D5 | H | A-38 |
| 52540 | m-D5 | H | m-D5 | m-D5 | H | A-39 |
| 52541 | m-D5 | H | m-D5 | m-D5 | H | A-40 |
| 52542 | m-D5 | H | m-D5 | m-D5 | H | A-43 |
| 52543 | m-D5 | H | m-D5 | m-D5 | H | A-44 |
| 52544 | m-D5 | H | m-D5 | m-D5 | H | A-45 |
| 52545 | m-D6 | H | m-D6 | m-D6 | H | A-6 |
| 52546 | m-D6 | H | m-D6 | m-D6 | H | A-7 |
| 52547 | m-D6 | H | m-D6 | m-D6 | H | A-10 |
| 52548 | m-D6 | H | m-D6 | m-D6 | H | A-12 |
| 52549 | m-D6 | H | m-D6 | m-D6 | H | A-14 |
| 52550 | m-D6 | H | m-D6 | m-D6 | H | A-21 |
| 52551 | m-D6 | H | m-D6 | m-D6 | H | A-27 |
| 52552 | m-D6 | H | m-D6 | m-D6 | H | A-30 |
| 52553 | m-D6 | H | m-D6 | m-D6 | H | A-31 |
| 52554 | m-D6 | H | m-D6 | m-D6 | H | A-34 |
| 52555 | m-D6 | H | m-D6 | m-D6 | H | A-38 |
| 52556 | m-D6 | H | m-D6 | m-D6 | H | A-39 |
| 52557 | m-D6 | H | m-D6 | m-D6 | H | A-40 |
| 52558 | m-D6 | H | m-D6 | m-D6 | H | A-43 |
| 52559 | m-D6 | H | m-D6 | m-D6 | H | A-44 |
| 52560 | m-D6 | H | m-D6 | m-D6 | H | A-45 |
| 52561 | m-D7 | H | m-D7 | m-D7 | H | A-6 |
| 52562 | m-D7 | H | m-D7 | m-D7 | H | A-7 |
| 52563 | m-D7 | H | m-D7 | m-D7 | H | A-10 |
| 52564 | m-D7 | H | m-D7 | m-D7 | H | A-12 |
| 52565 | m-D7 | H | m-D7 | m-D7 | H | A-14 |
| 52566 | m-D7 | H | m-D7 | m-D7 | H | A-21 |
| 52567 | m-D7 | H | m-D7 | m-D7 | H | A-27 |
| 52568 | m-D7 | H | m-D7 | m-D7 | H | A-30 |
| 52569 | m-D7 | H | m-D7 | m-D7 | H | A-31 |
| 52570 | m-D7 | H | m-D7 | m-D7 | H | A-34 |
| 52571 | m-D7 | H | m-D7 | m-D7 | H | A-38 |
| 52572 | m-D7 | H | m-D7 | m-D7 | H | A-39 |
| 52573 | m-D7 | H | m-D7 | m-D7 | H | A-40 |
| 52574 | m-D7 | H | m-D7 | m-D7 | H | A-43 |
| 52575 | m-D7 | H | m-D7 | m-D7 | H | A-44 |
| 52576 | m-D7 | H | m-D7 | m-D7 | H | A-45 |
| 52577 | m-D8 | H | m-D8 | m-D8 | H | A-6 |
| 52578 | m-D8 | H | m-D8 | m-D8 | H | A-7 |
| 52579 | m-D8 | H | m-D8 | m-D8 | H | A-10 |
| 52580 | m-D8 | H | m-D8 | m-D8 | H | A-12 |
| 52581 | m-D8 | H | m-D8 | m-D8 | H | A-14 |
| 52582 | m-D8 | H | m-D8 | m-D8 | H | A-21 |
| 52583 | m-D8 | H | m-D8 | m-D8 | H | A-27 |
| 52584 | m-D8 | H | m-D8 | m-D8 | H | A-30 |
| 52585 | m-D8 | H | m-D8 | m-D8 | H | A-31 |
| 52586 | m-D8 | H | m-D8 | m-D8 | H | A-34 |
| 52587 | m-D8 | H | m-D8 | m-D8 | H | A-38 |
| 52588 | m-D8 | H | m-D8 | m-D8 | H | A-39 |
| 52589 | m-D8 | H | m-D8 | m-D8 | H | A-40 |
| 52590 | m-D8 | H | m-D8 | m-D8 | H | A-43 |
| 52591 | m-D8 | H | m-D8 | m-D8 | H | A-44 |
| 52592 | m-D8 | H | m-D8 | m-D8 | H | A-45 |
| 52593 | m-D9 | H | m-D9 | m-D9 | H | A-6 |
| 52594 | m-D9 | H | m-D9 | m-D9 | H | A-7 |
| 52595 | m-D9 | H | m-D9 | m-D9 | H | A-10 |
| 52596 | m-D9 | H | m-D9 | m-D9 | H | A-12 |
| 52597 | m-D9 | H | m-D9 | m-D9 | H | A-14 |
| 52598 | m-D9 | H | m-D9 | m-D9 | H | A-21 |
| 52599 | m-D9 | H | m-D9 | m-D9 | H | A-27 |
| 52600 | m-D9 | H | m-D9 | m-D9 | H | A-30 |
| 52601 | m-D9 | H | m-D9 | m-D9 | H | A-31 |
| 52602 | m-D9 | H | m-D9 | m-D9 | H | A-34 |
| 52603 | m-D9 | H | m-D9 | m-D9 | H | A-38 |
| 52604 | m-D9 | H | m-D9 | m-D9 | H | A-39 |
| 52605 | m-D9 | H | m-D9 | m-D9 | H | A-40 |
| 52606 | m-D9 | H | m-D9 | m-D9 | H | A-43 |
| 52607 | m-D9 | H | m-D9 | m-D9 | H | A-44 |
| 52608 | m-D9 | H | m-D9 | m-D9 | H | A-45 |
| 52609 | m-D10 | H | m-D10 | m-D10 | H | A-6 |
| 52610 | m-D10 | H | m-D10 | m-D10 | H | A-7 |
| 52611 | m-D10 | H | m-D10 | m-D10 | H | A-10 |
| 52612 | m-D10 | H | m-D10 | m-D10 | H | A-12 |
| 52613 | m-D10 | H | m-D10 | m-D10 | H | A-14 |
| 52614 | m-D10 | H | m-D10 | m-D10 | H | A-21 |
| 52615 | m-D10 | H | m-D10 | m-D10 | H | A-27 |
| 52616 | m-D10 | H | m-D10 | m-D10 | H | A-30 |
| 52617 | m-D10 | H | m-D10 | m-D10 | H | A-31 |
| 52618 | m-D10 | H | m-D10 | m-D10 | H | A-34 |
| 52619 | m-D10 | H | m-D10 | m-D10 | H | A-38 |
| 52620 | m-D10 | H | m-D10 | m-D10 | H | A-39 |
| 52621 | m-D10 | H | m-D10 | m-D10 | H | A-40 |
| 52622 | m-D10 | H | m-D10 | m-D10 | H | A-43 |
| 52623 | m-D10 | H | m-D10 | m-D10 | H | A-44 |
| 52624 | m-D10 | H | m-D10 | m-D10 | H | A-45 |
| 52625 | m-D11 | H | m-D11 | m-D11 | H | A-6 |
| 52626 | m-D11 | H | m-D11 | m-D11 | H | A-7 |
| 52627 | m-D11 | H | m-D11 | m-D11 | H | A-10 |
| 52628 | m-D11 | H | m-D11 | m-D11 | H | A-12 |
| 52629 | m-D11 | H | m-D11 | m-D11 | H | A-14 |
| 52630 | m-D11 | H | m-D11 | m-D11 | H | A-21 |
| 52631 | m-D11 | H | m-D11 | m-D11 | H | A-27 |
| 52632 | m-D11 | H | m-D11 | m-D11 | H | A-30 |
| 52633 | m-D11 | H | m-D11 | m-D11 | H | A-31 |
| 52634 | m-D11 | H | m-D11 | m-D11 | H | A-34 |
| 52635 | m-D11 | H | m-D11 | m-D11 | H | A-38 |
| 52636 | m-D11 | H | m-D11 | m-D11 | H | A-39 |
| 52637 | m-D11 | H | m-D11 | m-D11 | H | A-40 |
| 52638 | m-D11 | H | m-D11 | m-D11 | H | A-43 |
| 52639 | m-D11 | H | m-D11 | m-D11 | H | A-44 |
| 52640 | m-D11 | H | m-D11 | m-D11 | H | A-45 |
| 52641 | m-D12 | H | m-D12 | m-D12 | H | A-6 |
| 52642 | m-D12 | H | m-D12 | m-D12 | H | A-7 |
| 52643 | m-D12 | H | m-D12 | m-D12 | H | A-10 |
| 52644 | m-D12 | H | m-D12 | m-D12 | H | A-12 |
| 52645 | m-D12 | H | m-D12 | m-D12 | H | A-14 |
| 52646 | m-D12 | H | m-D12 | m-D12 | H | A-21 |
| 52647 | m-D12 | H | m-D12 | m-D12 | H | A-27 |
| 52648 | m-D12 | H | m-D12 | m-D12 | H | A-30 |
| 52649 | m-D12 | H | m-D12 | m-D12 | H | A-31 |
| 52650 | m-D12 | H | m-D12 | m-D12 | H | A-34 |
| 52651 | m-D12 | H | m-D12 | m-D12 | H | A-38 |
| 52652 | m-D12 | H | m-D12 | m-D12 | H | A-39 |
| 52653 | m-D12 | H | m-D12 | m-D12 | H | A-40 |
| 52654 | m-D12 | H | m-D12 | m-D12 | H | A-43 |
| 52655 | m-D12 | H | m-D12 | m-D12 | H | A-44 |
| 52656 | m-D12 | H | m-D12 | m-D12 | H | A-45 |
| 52657 | m-D13 | H | m-D13 | m-D13 | H | A-6 |
| 52658 | m-D13 | H | m-D13 | m-D13 | H | A-7 |
| 52659 | m-D13 | H | m-D13 | m-D13 | H | A-10 |
| 52660 | m-D13 | H | m-D13 | m-D13 | H | A-12 |
| 52661 | m-D13 | H | m-D13 | m-D13 | H | A-14 |
| 52662 | m-D13 | H | m-D13 | m-D13 | H | A-21 |
| 52663 | m-D13 | H | m-D13 | m-D13 | H | A-27 |
| 52664 | m-D13 | H | m-D13 | m-D13 | H | A-30 |
| 52665 | m-D13 | H | m-D13 | m-D13 | H | A-31 |
| 52666 | m-D13 | H | m-D13 | m-D13 | H | A-34 |
| 52667 | m-D13 | H | m-D13 | m-D13 | H | A-38 |
| 52668 | m-D13 | H | m-D13 | m-D13 | H | A-39 |
| 52669 | m-D13 | H | m-D13 | m-D13 | H | A-40 |
| 52670 | m-D13 | H | m-D13 | m-D13 | H | A-43 |
| 52671 | m-D13 | H | m-D13 | m-D13 | H | A-44 |
| 52672 | m-D13 | H | m-D13 | m-D13 | H | A-45 |
| 52673 | m-D14 | H | m-D14 | m-D14 | H | A-6 |
| 52674 | m-D14 | H | m-D14 | m-D14 | H | A-7 |
| 52675 | m-D14 | H | m-D14 | m-D14 | H | A-10 |
| 52676 | m-D14 | H | m-D14 | m-D14 | H | A-12 |
| 52677 | m-D14 | H | m-D14 | m-D14 | H | A-14 |
| 52678 | m-D14 | H | m-D14 | m-D14 | H | A-21 |
| 52679 | m-D14 | H | m-D14 | m-D14 | H | A-27 |
| 52680 | m-D14 | H | m-D14 | m-D14 | H | A-30 |
| 52681 | m-D14 | H | m-D14 | m-D14 | H | A-31 |
| 52682 | m-D14 | H | m-D14 | m-D14 | H | A-34 |
| 52683 | m-D14 | H | m-D14 | m-D14 | H | A-38 |
| 52684 | m-D14 | H | m-D14 | m-D14 | H | A-39 |
| 52685 | m-D14 | H | m-D14 | m-D14 | H | A-40 |
| 52686 | m-D14 | H | m-D14 | m-D14 | H | A-43 |
| 52687 | m-D14 | H | m-D14 | m-D14 | H | A-44 |
| 52688 | m-D14 | H | m-D14 | m-D14 | H | A-45 |
| 52689 | H | m-D1 | m-D1 | m-D1 | H | A-6 |
| 52690 | H | m-D1 | m-D1 | m-D1 | H | A-7 |
| 52691 | H | m-D1 | m-D1 | m-D1 | H | A-10 |
| 52692 | H | m-D1 | m-D1 | m-D1 | H | A-12 |
| 52693 | H | m-D1 | m-D1 | m-D1 | H | A-14 |
| 52694 | H | m-D1 | m-D1 | m-D1 | H | A-21 |
| 52695 | H | m-D1 | m-D1 | m-D1 | H | A-27 |
| 52696 | H | m-D1 | m-D1 | m-D1 | H | A-30 |
| 52697 | H | m-D1 | m-D1 | m-D1 | H | A-31 |
| 52698 | H | m-D1 | m-D1 | m-D1 | H | A-34 |
| 52699 | H | m-D1 | m-D1 | m-D1 | H | A-38 |
| 52700 | H | m-D1 | m-D1 | m-D1 | H | A-39 |
| 52701 | H | m-D1 | m-D1 | m-D1 | H | A-40 |
| 52702 | H | m-D1 | m-D1 | m-D1 | H | A-43 |
| 52703 | H | m-D1 | m-D1 | m-D1 | H | A-44 |
| 52704 | H | m-D1 | m-D1 | m-D1 | H | A-45 |
| 52705 | H | m-D2 | m-D2 | m-D2 | H | A-6 |
| 52706 | H | m-D2 | m-D2 | m-D2 | H | A-7 |
| 52707 | H | m-D2 | m-D2 | m-D2 | H | A-10 |
| 52708 | H | m-D2 | m-D2 | m-D2 | H | A-12 |
| 52709 | H | m-D2 | m-D2 | m-D2 | H | A-14 |
| 52710 | H | m-D2 | m-D2 | m-D2 | H | A-21 |
| 52711 | H | m-D2 | m-D2 | m-D2 | H | A-27 |
| 52712 | H | m-D2 | m-D2 | m-D2 | H | A-30 |
| 52713 | H | m-D2 | m-D2 | m-D2 | H | A-31 |
| 52714 | H | m-D2 | m-D2 | m-D2 | H | A-34 |
| 52715 | H | m-D2 | m-D2 | m-D2 | H | A-38 |
| 52716 | H | m-D2 | m-D2 | m-D2 | H | A-39 |
| 52717 | H | m-D2 | m-D2 | m-D2 | H | A-40 |
| 52718 | H | m-D2 | m-D2 | m-D2 | H | A-43 |
| 52719 | H | m-D2 | m-D2 | m-D2 | H | A-44 |
| 52720 | H | m-D2 | m-D2 | m-D2 | H | A-45 |
| 52721 | H | m-D3 | m-D3 | m-D3 | H | A-6 |
| 52722 | H | m-D3 | m-D3 | m-D3 | H | A-7 |
| 52723 | H | m-D3 | m-D3 | m-D3 | H | A-10 |
| 52724 | H | m-D3 | m-D3 | m-D3 | H | A-12 |
| 52725 | H | m-D3 | m-D3 | m-D3 | H | A-14 |
| 52726 | H | m-D3 | m-D3 | m-D3 | H | A-21 |
| 52727 | H | m-D3 | m-D3 | m-D3 | H | A-27 |
| 52728 | H | m-D3 | m-D3 | m-D3 | H | A-30 |
| 52729 | H | m-D3 | m-D3 | m-D3 | H | A-31 |
| 52730 | H | m-D3 | m-D3 | m-D3 | H | A-34 |
| 52731 | H | m-D3 | m-D3 | m-D3 | H | A-38 |
| 52732 | H | m-D3 | m-D3 | m-D3 | H | A-39 |
| 52733 | H | m-D3 | m-D3 | m-D3 | H | A-40 |
| 52734 | H | m-D3 | m-D3 | m-D3 | H | A-43 |
| 52735 | H | m-D3 | m-D3 | m-D3 | H | A-44 |
| 52736 | H | m-D3 | m-D3 | m-D3 | H | A-45 |
| 52737 | H | m-D4 | m-D4 | m-D4 | H | A-6 |
| 52738 | H | m-D4 | m-D4 | m-D4 | H | A-7 |
| 52739 | H | m-D4 | m-D4 | m-D4 | H | A-10 |
| 52740 | H | m-D4 | m-D4 | m-D4 | H | A-12 |
| 52741 | H | m-D4 | m-D4 | m-D4 | H | A-14 |
| 52742 | H | m-D4 | m-D4 | m-D4 | H | A-21 |
| 52743 | H | m-D4 | m-D4 | m-D4 | H | A-27 |
| 52744 | H | m-D4 | m-D4 | m-D4 | H | A-30 |
| 52745 | H | m-D4 | m-D4 | m-D4 | H | A-31 |
| 52746 | H | m-D4 | m-D4 | m-D4 | H | A-34 |
| 52747 | H | m-D4 | m-D4 | m-D4 | H | A-38 |
| 52748 | H | m-D4 | m-D4 | m-D4 | H | A-39 |
| 52749 | H | m-D4 | m-D4 | m-D4 | H | A-40 |
| 52750 | H | m-D4 | m-D4 | m-D4 | H | A-43 |
| 52751 | H | m-D4 | m-D4 | m-D4 | H | A-44 |
| 52752 | H | m-D4 | m-D4 | m-D4 | H | A-45 |
| 52753 | H | m-D5 | m-D5 | m-D5 | H | A-6 |
| 52754 | H | m-D5 | m-D5 | m-D5 | H | A-7 |
| 52755 | H | m-D5 | m-D5 | m-D5 | H | A-10 |
| 52756 | H | m-D5 | m-D5 | m-D5 | H | A-12 |
| 52757 | H | m-D5 | m-D5 | m-D5 | H | A-14 |
| 52758 | H | m-D5 | m-D5 | m-D5 | H | A-21 |
| 52759 | H | m-D5 | m-D5 | m-D5 | H | A-27 |
| 52760 | H | m-D5 | m-D5 | m-D5 | H | A-30 |
| 52761 | H | m-D5 | m-D5 | m-D5 | H | A-31 |
| 52762 | H | m-D5 | m-D5 | m-D5 | H | A-34 |
| 52763 | H | m-D5 | m-D5 | m-D5 | H | A-38 |
| 52764 | H | m-D5 | m-D5 | m-D5 | H | A-39 |
| 52765 | H | m-D5 | m-D5 | m-D5 | H | A-40 |
| 52766 | H | m-D5 | m-D5 | m-D5 | H | A-43 |
| 52767 | H | m-D5 | m-D5 | m-D5 | H | A-44 |
| 52768 | H | m-D5 | m-D5 | m-D5 | H | A-45 |
| 52769 | H | m-D6 | m-D6 | m-D6 | H | A-6 |
| 52770 | H | m-D6 | m-D6 | m-D6 | H | A-7 |
| 52771 | H | m-D6 | m-D6 | m-D6 | H | A-10 |
| 52772 | H | m-D6 | m-D6 | m-D6 | H | A-12 |
| 52773 | H | m-D6 | m-D6 | m-D6 | H | A-14 |
| 52774 | H | m-D6 | m-D6 | m-D6 | H | A-21 |
| 52775 | H | m-D6 | m-D6 | m-D6 | H | A-27 |
| 52776 | H | m-D6 | m-D6 | m-D6 | H | A-30 |
| 52777 | H | m-D6 | m-D6 | m-D6 | H | A-31 |
| 52778 | H | m-D6 | m-D6 | m-D6 | H | A-34 |
| 52779 | H | m-D6 | m-D6 | m-D6 | H | A-38 |
| 52780 | H | m-D6 | m-D6 | m-D6 | H | A-39 |
| 52781 | H | m-D6 | m-D6 | m-D6 | H | A-40 |
| 52782 | H | m-D6 | m-D6 | m-D6 | H | A-43 |
| 52783 | H | m-D6 | m-D6 | m-D6 | H | A-44 |
| 52784 | H | m-D6 | m-D6 | m-D6 | H | A-45 |
| 52785 | H | m-D7 | m-D7 | m-D7 | H | A-6 |
| 52786 | H | m-D7 | m-D7 | m-D7 | H | A-7 |
| 52787 | H | m-D7 | m-D7 | m-D7 | H | A-10 |
| 52788 | H | m-D7 | m-D7 | m-D7 | H | A-12 |
| 52789 | H | m-D7 | m-D7 | m-D7 | H | A-14 |
| 52790 | H | m-D7 | m-D7 | m-D7 | H | A-21 |
| 52791 | H | m-D7 | m-D7 | m-D7 | H | A-27 |
| 52792 | H | m-D7 | m-D7 | m-D7 | H | A-30 |
| 52793 | H | m-D7 | m-D7 | m-D7 | H | A-31 |
| 52794 | H | m-D7 | m-D7 | m-D7 | H | A-34 |
| 52795 | H | m-D7 | m-D7 | m-D7 | H | A-38 |
| 52796 | H | m-D7 | m-D7 | m-D7 | H | A-39 |
| 52797 | H | m-D7 | m-D7 | m-D7 | H | A-40 |
| 52798 | H | m-D7 | m-D7 | m-D7 | H | A-43 |
| 52799 | H | m-D7 | m-D7 | m-D7 | H | A-44 |
| 52800 | H | m-D7 | m-D7 | m-D7 | H | A-45 |
| 52801 | H | m-D8 | m-D8 | m-D8 | H | A-6 |
| 52802 | H | m-D8 | m-D8 | m-D8 | H | A-7 |
| 52803 | H | m-D8 | m-D8 | m-D8 | H | A-10 |
| 52804 | H | m-D8 | m-D8 | m-D8 | H | A-12 |
| 52805 | H | m-D8 | m-D8 | m-D8 | H | A-14 |
| 52806 | H | m-D8 | m-D8 | m-D8 | H | A-21 |
| 52807 | H | m-D8 | m-D8 | m-D8 | H | A-27 |
| 52808 | H | m-D8 | m-D8 | m-D8 | H | A-30 |
| 52809 | H | m-D8 | m-D8 | m-D8 | H | A-31 |
| 52810 | H | m-D8 | m-D8 | m-D8 | H | A-34 |
| 52811 | H | m-D8 | m-D8 | m-D8 | H | A-38 |
| 52812 | H | m-D8 | m-D8 | m-D8 | H | A-39 |
| 52813 | H | m-D8 | m-D8 | m-D8 | H | A-40 |
| 52814 | H | m-D8 | m-D8 | m-D8 | H | A-43 |
| 52815 | H | m-D8 | m-D8 | m-D8 | H | A-44 |
| 52816 | H | m-D8 | m-D8 | m-D8 | H | A-45 |
| 52817 | H | m-D9 | m-D9 | m-D9 | H | A-6 |
| 52818 | H | m-D9 | m-D9 | m-D9 | H | A-7 |
| 52819 | H | m-D9 | m-D9 | m-D9 | H | A-10 |
| 52820 | H | m-D9 | m-D9 | m-D9 | H | A-12 |
| 52821 | H | m-D9 | m-D9 | m-D9 | H | A-14 |
| 52822 | H | m-D9 | m-D9 | m-D9 | H | A-21 |
| 52823 | H | m-D9 | m-D9 | m-D9 | H | A-27 |
| 52824 | H | m-D9 | m-D9 | m-D9 | H | A-30 |
| 52825 | H | m-D9 | m-D9 | m-D9 | H | A-31 |
| 52826 | H | m-D9 | m-D9 | m-D9 | H | A-34 |
| 52827 | H | m-D9 | m-D9 | m-D9 | H | A-38 |
| 52828 | H | m-D9 | m-D9 | m-D9 | H | A-39 |
| 52829 | H | m-D9 | m-D9 | m-D9 | H | A-40 |
| 52830 | H | m-D9 | m-D9 | m-D9 | H | A-43 |
| 52831 | H | m-D9 | m-D9 | m-D9 | H | A-44 |
| 52832 | H | m-D9 | m-D9 | m-D9 | H | A-45 |
| 52833 | H | m-D10 | m-D10 | m-D10 | H | A-6 |
| 52834 | H | m-D10 | m-D10 | m-D10 | H | A-7 |
| 52835 | H | m-D10 | m-D10 | m-D10 | H | A-10 |
| 52836 | H | m-D10 | m-D10 | m-D10 | H | A-12 |
| 52837 | H | m-D10 | m-D10 | m-D10 | H | A-14 |
| 52838 | H | m-D10 | m-D10 | m-D10 | H | A-21 |
| 52839 | H | m-D10 | m-D10 | m-D10 | H | A-27 |
| 52840 | H | m-D10 | m-D10 | m-D10 | H | A-30 |
| 52841 | H | m-D10 | m-D10 | m-D10 | H | A-31 |
| 52842 | H | m-D10 | m-D10 | m-D10 | H | A-34 |
| 52843 | H | m-D10 | m-D10 | m-D10 | H | A-38 |
| 52844 | H | m-D10 | m-D10 | m-D10 | H | A-39 |
| 52845 | H | m-D10 | m-D10 | m-D10 | H | A-40 |
| 52846 | H | m-D10 | m-D10 | m-D10 | H | A-43 |
| 52847 | H | m-D10 | m-D10 | m-D10 | H | A-44 |
| 52848 | H | m-D10 | m-D10 | m-D10 | H | A-45 |
| 52849 | H | m-D11 | m-D11 | m-D11 | H | A-6 |
| 52850 | H | m-D11 | m-D11 | m-D11 | H | A-7 |
| 52851 | H | m-D11 | m-D11 | m-D11 | H | A-10 |
| 52852 | H | m-D11 | m-D11 | m-D11 | H | A-12 |
| 52853 | H | m-D11 | m-D11 | m-D11 | H | A-14 |
| 52854 | H | m-D11 | m-D11 | m-D11 | H | A-21 |
| 52855 | H | m-D11 | m-D11 | m-D11 | H | A-27 |
| 52856 | H | m-D11 | m-D11 | m-D11 | H | A-30 |
| 52857 | H | m-D11 | m-D11 | m-D11 | H | A-31 |
| 52858 | H | m-D11 | m-D11 | m-D11 | H | A-34 |
| 52859 | H | m-D11 | m-D11 | m-D11 | H | A-38 |
| 52860 | H | m-D11 | m-D11 | m-D11 | H | A-39 |
| 52861 | H | m-D11 | m-D11 | m-D11 | H | A-40 |
| 52862 | H | m-D11 | m-D11 | m-D11 | H | A-43 |
| 52863 | H | m-D11 | m-D11 | m-D11 | H | A-44 |
| 52864 | H | m-D11 | m-D11 | m-D11 | H | A-45 |
| 52865 | H | m-D12 | m-D12 | m-D12 | H | A-6 |
| 52866 | H | m-D12 | m-D12 | m-D12 | H | A-7 |
| 52867 | H | m-D12 | m-D12 | m-D12 | H | A-10 |
| 52868 | H | m-D12 | m-D12 | m-D12 | H | A-12 |
| 52869 | H | m-D12 | m-D12 | m-D12 | H | A-14 |
| 52870 | H | m-D12 | m-D12 | m-D12 | H | A-21 |
| 52871 | H | m-D12 | m-D12 | m-D12 | H | A-27 |
| 52872 | H | m-D12 | m-D12 | m-D12 | H | A-30 |
| 52873 | H | m-D12 | m-D12 | m-D12 | H | A-31 |
| 52874 | H | m-D12 | m-D12 | m-D12 | H | A-34 |
| 52875 | H | m-D12 | m-D12 | m-D12 | H | A-38 |
| 52876 | H | m-D12 | m-D12 | m-D12 | H | A-39 |
| 52877 | H | m-D12 | m-D12 | m-D12 | H | A-40 |
| 52878 | H | m-D12 | m-D12 | m-D12 | H | A-43 |
| 52879 | H | m-D12 | m-D12 | m-D12 | H | A-44 |
| 52880 | H | m-D12 | m-D12 | m-D12 | H | A-45 |
| 52881 | H | m-D13 | m-D13 | m-D13 | H | A-6 |
| 52882 | H | m-D13 | m-D13 | m-D13 | H | A-7 |
| 52883 | H | m-D13 | m-D13 | m-D13 | H | A-10 |
| 52884 | H | m-D13 | m-D13 | m-D13 | H | A-12 |
| 52885 | H | m-D13 | m-D13 | m-D13 | H | A-14 |
| 52886 | H | m-D13 | m-D13 | m-D13 | H | A-21 |
| 52887 | H | m-D13 | m-D13 | m-D13 | H | A-27 |
| 52888 | H | m-D13 | m-D13 | m-D13 | H | A-30 |
| 52889 | H | m-D13 | m-D13 | m-D13 | H | A-31 |
| 52890 | H | m-D13 | m-D13 | m-D13 | H | A-34 |
| 52891 | H | m-D13 | m-D13 | m-D13 | H | A-38 |
| 52892 | H | m-D13 | m-D13 | m-D13 | H | A-39 |
| 52893 | H | m-D13 | m-D13 | m-D13 | H | A-40 |
| 52894 | H | m-D13 | m-D13 | m-D13 | H | A-43 |
| 52895 | H | m-D13 | m-D13 | m-D13 | H | A-44 |
| 52896 | H | m-D13 | m-D13 | m-D13 | H | A-45 |
| 52897 | H | m-D14 | m-D14 | m-D14 | H | A-6 |
| 52898 | H | m-D14 | m-D14 | m-D14 | H | A-7 |
| 52899 | H | m-D14 | m-D14 | m-D14 | H | A-10 |
| 52900 | H | m-D14 | m-D14 | m-D14 | H | A-12 |
| 52901 | H | m-D14 | m-D14 | m-D14 | H | A-14 |
| 52902 | H | m-D14 | m-D14 | m-D14 | H | A-21 |
| 52903 | H | m-D14 | m-D14 | m-D14 | H | A-27 |
| 52904 | H | m-D14 | m-D14 | m-D14 | H | A-30 |
| 52905 | H | m-D14 | m-D14 | m-D14 | H | A-31 |
| 52906 | H | m-D14 | m-D14 | m-D14 | H | A-34 |
| 52907 | H | m-D14 | m-D14 | m-D14 | H | A-38 |
| 52908 | H | m-D14 | m-D14 | m-D14 | H | A-39 |
| 52909 | H | m-D14 | m-D14 | m-D14 | H | A-40 |
| 52910 | H | m-D14 | m-D14 | m-D14 | H | A-43 |
| 52911 | H | m-D14 | m-D14 | m-D14 | H | A-44 |
| 52912 | H | m-D14 | m-D14 | m-D14 | H | A-45 |
| 52913 | m-D1 | m-D1 | H | H | m-D1 | A-6 |
| 52914 | m-D1 | m-D1 | H | H | m-D1 | A-7 |
| 52915 | m-D1 | m-D1 | H | H | m-D1 | A-10 |
| 52916 | m-D1 | m-D1 | H | H | m-D1 | A-12 |
| 52917 | m-D1 | m-D1 | H | H | m-D1 | A-14 |
| 52918 | m-D1 | m-D1 | H | H | m-D1 | A-21 |
| 52919 | m-D1 | m-D1 | H | H | m-D1 | A-27 |
| 52920 | m-D1 | m-D1 | H | H | m-D1 | A-30 |
| 52921 | m-D1 | m-D1 | H | H | m-D1 | A-31 |
| 52922 | m-D1 | m-D1 | H | H | m-D1 | A-34 |
| 52923 | m-D1 | m-D1 | H | H | m-D1 | A-38 |
| 52924 | m-D1 | m-D1 | H | H | m-D1 | A-39 |
| 52925 | m-D1 | m-D1 | H | H | m-D1 | A-40 |
| 52926 | m-D1 | m-D1 | H | H | m-D1 | A-43 |
| 52927 | m-D1 | m-D1 | H | H | m-D1 | A-44 |
| 52928 | m-D1 | m-D1 | H | H | m-D1 | A-45 |
| 52929 | m-D2 | m-D2 | H | H | m-D2 | A-6 |
| 52930 | m-D2 | m-D2 | H | H | m-D2 | A-7 |
| 52931 | m-D2 | m-D2 | H | H | m-D2 | A-10 |
| 52932 | m-D2 | m-D2 | H | H | m-D2 | A-12 |
| 52933 | m-D2 | m-D2 | H | H | m-D2 | A-14 |
| 52934 | m-D2 | m-D2 | H | H | m-D2 | A-21 |
| 52935 | m-D2 | m-D2 | H | H | m-D2 | A-27 |
| 52936 | m-D2 | m-D2 | H | H | m-D2 | A-30 |
| 52937 | m-D2 | m-D2 | H | H | m-D2 | A-31 |
| 52938 | m-D2 | m-D2 | H | H | m-D2 | A-34 |
| 52939 | m-D2 | m-D2 | H | H | m-D2 | A-38 |
| 52940 | m-D2 | m-D2 | H | H | m-D2 | A-39 |
| 52941 | m-D2 | m-D2 | H | H | m-D2 | A-40 |
| 52942 | m-D2 | m-D2 | H | H | m-D2 | A-43 |
| 52943 | m-D2 | m-D2 | H | H | m-D2 | A-44 |
| 52944 | m-D2 | m-D2 | H | H | m-D2 | A-45 |
| 52945 | m-D3 | m-D3 | H | H | m-D3 | A-6 |
| 52946 | m-D3 | m-D3 | H | H | m-D3 | A-7 |
| 52947 | m-D3 | m-D3 | H | H | m-D3 | A-10 |
| 52948 | m-D3 | m-D3 | H | H | m-D3 | A-12 |
| 52949 | m-D3 | m-D3 | H | H | m-D3 | A-14 |
| 52950 | m-D3 | m-D3 | H | H | m-D3 | A-21 |
| 52951 | m-D3 | m-D3 | H | H | m-D3 | A-27 |
| 52952 | m-D3 | m-D3 | H | H | m-D3 | A-30 |
| 52953 | m-D3 | m-D3 | H | H | m-D3 | A-31 |
| 52954 | m-D3 | m-D3 | H | H | m-D3 | A-34 |
| 52955 | m-D3 | m-D3 | H | H | m-D3 | A-38 |
| 52956 | m-D3 | m-D3 | H | H | m-D3 | A-39 |
| 52957 | m-D3 | m-D3 | H | H | m-D3 | A-40 |
| 52958 | m-D3 | m-D3 | H | H | m-D3 | A-43 |
| 52959 | m-D3 | m-D3 | H | H | m-D3 | A-44 |
| 52960 | m-D3 | m-D3 | H | H | m-D3 | A-45 |
| 52961 | m-D4 | m-D4 | H | H | m-D4 | A-6 |
| 52962 | m-D4 | m-D4 | H | H | m-D4 | A-7 |
| 52963 | m-D4 | m-D4 | H | H | m-D4 | A-10 |
| 52964 | m-D4 | m-D4 | H | H | m-D4 | A-12 |
| 52965 | m-D4 | m-D4 | H | H | m-D4 | A-14 |
| 52966 | m-D4 | m-D4 | H | H | m-D4 | A-21 |
| 52967 | m-D4 | m-D4 | H | H | m-D4 | A-27 |
| 52968 | m-D4 | m-D4 | H | H | m-D4 | A-30 |
| 52969 | m-D4 | m-D4 | H | H | m-D4 | A-31 |
| 52970 | m-D4 | m-D4 | H | H | m-D4 | A-34 |
| 52971 | m-D4 | m-D4 | H | H | m-D4 | A-38 |
| 52972 | m-D4 | m-D4 | H | H | m-D4 | A-39 |
| 52973 | m-D4 | m-D4 | H | H | m-D4 | A-40 |
| 52974 | m-D4 | m-D4 | H | H | m-D4 | A-43 |
| 52975 | m-D4 | m-D4 | H | H | m-D4 | A-44 |
| 52976 | m-D4 | m-D4 | H | H | m-D4 | A-45 |
| 52977 | m-D5 | m-D5 | H | H | m-D5 | A-6 |
| 52978 | m-D5 | m-D5 | H | H | m-D5 | A-7 |
| 52979 | m-D5 | m-D5 | H | H | m-D5 | A-10 |
| 52980 | m-D5 | m-D5 | H | H | m-D5 | A-12 |
| 52981 | m-D5 | m-D5 | H | H | m-D5 | A-14 |
| 52982 | m-D5 | m-D5 | H | H | m-D5 | A-21 |
| 52983 | m-D5 | m-D5 | H | H | m-D5 | A-27 |
| 52984 | m-D5 | m-D5 | H | H | m-D5 | A-30 |
| 52985 | m-D5 | m-D5 | H | H | m-D5 | A-31 |
| 52986 | m-D5 | m-D5 | H | H | m-D5 | A-34 |
| 52987 | m-D5 | m-D5 | H | H | m-D5 | A-38 |
| 52988 | m-D5 | m-D5 | H | H | m-D5 | A-39 |
| 52989 | m-D5 | m-D5 | H | H | m-D5 | A-40 |
| 52990 | m-D5 | m-D5 | H | H | m-D5 | A-43 |
| 52991 | m-D5 | m-D5 | H | H | m-D5 | A-44 |
| 52992 | m-D5 | m-D5 | H | H | m-D5 | A-45 |
| 52993 | m-D6 | m-D6 | H | H | m-D6 | A-6 |
| 52994 | m-D6 | m-D6 | H | H | m-D6 | A-7 |
| 52995 | m-D6 | m-D6 | H | H | m-D6 | A-10 |
| 52996 | m-D6 | m-D6 | H | H | m-D6 | A-12 |
| 52997 | m-D6 | m-D6 | H | H | m-D6 | A-14 |
| 52998 | m-D6 | m-D6 | H | H | m-D6 | A-21 |
| 52999 | m-D6 | m-D6 | H | H | m-D6 | A-27 |
| 53000 | m-D6 | m-D6 | H | H | m-D6 | A-30 |
| 53001 | m-D6 | m-D6 | H | H | m-D6 | A-31 |
| 53002 | m-D6 | m-D6 | H | H | m-D6 | A-34 |
| 53003 | m-D6 | m-D6 | H | H | m-D6 | A-38 |
| 53004 | m-D6 | m-D6 | H | H | m-D6 | A-39 |
| 53005 | m-D6 | m-D6 | H | H | m-D6 | A-40 |
| 53006 | m-D6 | m-D6 | H | H | m-D6 | A-43 |
| 53007 | m-D6 | m-D6 | H | H | m-D6 | A-44 |
| 63008 | m-D6 | m-D6 | H | H | m-D6 | A-45 |
| 53009 | m-D7 | m-D7 | H | H | m-D7 | A-6 |
| 53010 | m-D7 | m-D7 | H | H | m-D7 | A-7 |
| 53011 | m-D7 | m-D7 | H | H | m-D7 | A-10 |
| 53012 | m-D7 | m-D7 | H | H | m-D7 | A-12 |
| 53013 | m-D7 | m-D7 | H | H | m-D7 | A-14 |
| 53014 | m-D7 | m-D7 | H | H | m-D7 | A-21 |
| 53015 | m-D7 | m-D7 | H | H | m-D7 | A-27 |
| 53016 | m-D7 | m-D7 | H | H | m-D7 | A-30 |
| 53017 | m-D7 | m-D7 | H | H | m-D7 | A-31 |
| 53018 | m-D7 | m-D7 | H | H | m-D7 | A-34 |
| 53019 | m-D7 | m-D7 | H | H | m-D7 | A-38 |
| 53020 | m-D7 | m-D7 | H | H | m-D7 | A-39 |
| 53021 | m-D7 | m-D7 | H | H | m-D7 | A-40 |
| 53022 | m-D7 | m-D7 | H | H | m-D7 | A-43 |
| 53023 | m-D7 | m-D7 | H | H | m-D7 | A-44 |
| 53024 | m-D7 | m-D7 | H | H | m-D7 | A-45 |
| 53025 | m-D8 | m-D8 | H | H | m-D8 | A-6 |
| 53026 | m-D8 | m-D8 | H | H | m-D8 | A-7 |
| 53027 | m-D8 | m-D8 | H | H | m-D8 | A-10 |
| 53028 | m-D8 | m-D8 | H | H | m-D8 | A-12 |
| 53029 | m-D8 | m-D8 | H | H | m-D8 | A-14 |
| 53030 | m-D8 | m-D8 | H | H | m-D8 | A-21 |
| 53031 | m-D8 | m-D8 | H | H | m-D8 | A-27 |
| 53032 | m-D8 | m-D8 | H | H | m-D8 | A-30 |
| 53033 | m-D8 | m-D8 | H | H | m-D8 | A-31 |
| 53034 | m-D8 | m-D8 | H | H | m-D8 | A-34 |
| 53035 | m-D8 | m-D8 | H | H | m-D8 | A-38 |
| 53036 | m-D8 | m-D8 | H | H | m-D8 | A-39 |
| 53037 | m-D8 | m-D8 | H | H | m-D8 | A-40 |
| 53038 | m-D8 | m-D8 | H | H | m-D8 | A-43 |
| 53039 | m-D8 | m-D8 | H | H | m-D8 | A-44 |
| 53040 | m-D8 | m-D8 | H | H | m-D8 | A-45 |
| 53041 | m-D9 | m-D9 | H | H | m-D9 | A-6 |
| 53042 | m-D9 | m-D9 | H | H | m-D9 | A-7 |
| 53043 | m-D9 | m-D9 | H | H | m-D9 | A-10 |
| 53044 | m-D9 | m-D9 | H | H | m-D9 | A-12 |
| 53045 | m-D9 | m-D9 | H | H | m-D9 | A-14 |
| 53046 | m-D9 | m-D9 | H | H | m-D9 | A-21 |
| 53047 | m-D9 | m-D9 | H | H | m-D9 | A-27 |
| 53048 | m-D9 | m-D9 | H | H | m-D9 | A-30 |
| 53049 | m-D9 | m-D9 | H | H | m-D9 | A-31 |
| 53050 | m-D9 | m-D9 | H | H | m-D9 | A-34 |
| 53051 | m-D9 | m-D9 | H | H | m-D9 | A-38 |
| 53052 | m-D9 | m-D9 | H | H | m-D9 | A-39 |
| 53053 | m-D9 | m-D9 | H | H | m-D9 | A-40 |
| 53054 | m-D9 | m-D9 | H | H | m-D9 | A-43 |
| 53055 | m-D9 | m-D9 | H | H | m-D9 | A-44 |
| 53056 | m-D9 | m-D9 | H | H | m-D9 | A-45 |
| 53057 | m-D10 | m-D10 | H | H | m-D10 | A-6 |
| 53058 | m-D10 | m-D10 | H | H | m-D10 | A-7 |
| 53059 | m-D10 | m-D10 | H | H | m-D10 | A-10 |
| 53060 | m-D10 | m-D10 | H | H | m-D10 | A-12 |
| 53061 | m-D10 | m-D10 | H | H | m-D10 | A-14 |
| 53062 | m-D10 | m-D10 | H | H | m-D10 | A-21 |
| 53063 | m-D10 | m-D10 | H | H | m-D10 | A-27 |
| 53064 | m-D10 | m-D10 | H | H | m-D10 | A-30 |
| 53065 | m-D10 | m-D10 | H | H | m-D10 | A-31 |
| 53066 | m-D10 | m-D10 | H | H | m-D10 | A-34 |
| 53067 | m-D10 | m-D10 | H | H | m-D10 | A-38 |
| 53068 | m-D10 | m-D10 | H | H | m-D10 | A-39 |
| 53069 | m-D10 | m-D10 | H | H | m-D10 | A-40 |
| 53070 | m-D10 | m-D10 | H | H | m-D10 | A-43 |
| 53071 | m-D10 | m-D10 | H | H | m-D10 | A-44 |
| 53072 | m-D10 | m-D10 | H | H | m-D10 | A-45 |
| 53073 | m-D11 | m-D11 | H | H | m-D11 | A-6 |
| 53074 | m-D11 | m-D11 | H | H | m-D11 | A-7 |
| 53075 | m-D11 | m-D11 | H | H | m-D11 | A-10 |
| 53076 | m-D11 | m-D11 | H | H | m-D11 | A-12 |
| 53077 | m-D11 | m-D11 | H | H | m-D11 | A-14 |
| 53078 | m-D11 | m-D11 | H | H | m-D11 | A-21 |
| 53079 | m-D11 | m-D11 | H | H | m-D11 | A-27 |
| 53080 | m-D11 | m-D11 | H | H | m-D11 | A-30 |
| 53081 | m-D11 | m-D11 | H | H | m-D11 | A-31 |
| 53082 | m-D11 | m-D11 | H | H | m-D11 | A-34 |
| 53083 | m-D11 | m-D11 | H | H | m-D11 | A-38 |
| 53084 | m-D11 | m-D11 | H | H | m-D11 | A-39 |
| 53085 | m-D11 | m-D11 | H | H | m-D11 | A-40 |
| 53086 | m-D11 | m-D11 | H | H | m-D11 | A-43 |
| 53087 | m-D11 | m-D11 | H | H | m-D11 | A-44 |
| 53088 | m-D11 | m-D11 | H | H | m-D11 | A-45 |
| 53089 | m-D12 | m-D12 | H | H | m-D12 | A-6 |
| 53090 | m-D12 | m-D12 | H | H | m-D12 | A-7 |
| 53091 | m-D12 | m-D12 | H | H | m-D12 | A-10 |
| 53092 | m-D12 | m-D12 | H | H | m-D12 | A-12 |
| 53093 | m-D12 | m-D12 | H | H | m-D12 | A-14 |
| 53094 | m-D12 | m-D12 | H | H | m-D12 | A-21 |
| 53095 | m-D12 | m-D12 | H | H | m-D12 | A-27 |
| 53096 | m-D12 | m-D12 | H | H | m-D12 | A-30 |
| 53097 | m-D12 | m-D12 | H | H | m-D12 | A-31 |
| 53098 | m-D12 | m-D12 | H | H | m-D12 | A-34 |
| 53099 | m-D12 | m-D12 | H | H | m-D12 | A-38 |
| 53100 | m-D12 | m-D12 | H | H | m-D12 | A-39 |
| 53101 | m-D12 | m-D12 | H | H | m-D12 | A-40 |
| 53102 | m-D12 | m-D12 | H | H | m-D12 | A-43 |
| 53103 | m-D12 | m-D12 | H | H | m-D12 | A-44 |
| 53104 | m-D12 | m-D12 | H | H | m-D12 | A-45 |
| 53105 | m-D13 | m-D13 | H | H | m-D13 | A-6 |
| 53106 | m-D13 | m-D13 | H | H | m-D13 | A-7 |
| 53107 | m-D13 | m-D13 | H | H | m-D13 | A-10 |
| 53108 | m-D13 | m-D13 | H | H | m-D13 | A-12 |
| 53109 | m-D13 | m-D13 | H | H | m-D13 | A-14 |
| 53110 | m-D13 | m-D13 | H | H | m-D13 | A-21 |
| 53111 | m-D13 | m-D13 | H | H | m-D13 | A-27 |
| 53112 | m-D13 | m-D13 | H | H | m-D13 | A-30 |
| 53113 | m-D13 | m-D13 | H | H | m-D13 | A-31 |
| 53114 | m-D13 | m-D13 | H | H | m-D13 | A-34 |
| 53115 | m-D13 | m-D13 | H | H | m-D13 | A-38 |
| 53116 | m-D13 | m-D13 | H | H | m-D13 | A-39 |
| 53117 | m-D13 | m-D13 | H | H | m-D13 | A-40 |
| 53118 | m-D13 | m-D13 | H | H | m-D13 | A-43 |
| 53119 | m-D13 | m-D13 | H | H | m-D13 | A-44 |
| 53120 | m-D13 | m-D13 | H | H | m-D13 | A-45 |
| 53121 | m-D14 | m-D14 | H | H | m-D14 | A-6 |
| 53122 | m-D14 | m-D14 | H | H | m-D14 | A-7 |
| 53123 | m-D14 | m-D14 | H | H | m-D14 | A-10 |
| 53124 | m-D14 | m-D14 | H | H | m-D14 | A-12 |
| 53125 | m-D14 | m-D14 | H | H | m-D14 | A-14 |
| 53126 | m-D14 | m-D14 | H | H | m-D14 | A-21 |
| 53127 | m-D14 | m-D14 | H | H | m-D14 | A-27 |
| 53128 | m-D14 | m-D14 | H | H | m-D14 | A-30 |
| 53129 | m-D14 | m-D14 | H | H | m-D14 | A-31 |
| 53130 | m-D14 | m-D14 | H | H | m-D14 | A-34 |
| 53131 | m-D14 | m-D14 | H | H | m-D14 | A-38 |
| 53132 | m-D14 | m-D14 | H | H | m-D14 | A-39 |
| 53133 | m-D14 | m-D14 | H | H | m-D14 | A-40 |
| 53134 | m-D14 | m-D14 | H | H | m-D14 | A-43 |
| 53135 | m-D14 | m-D14 | H | H | m-D14 | A-44 |
| 53136 | m-D14 | m-D14 | H | H | m-D14 | A-45 |
| 53137 | m-D1 | H | m-D1 | H | m-D1 | A-6 |
| 53138 | m-D1 | H | m-D1 | H | m-D1 | A-7 |
| 53139 | m-D1 | H | m-D1 | H | m-D1 | A-10 |
| 53140 | m-D1 | H | m-D1 | H | m-D1 | A-12 |
| 53141 | m-D1 | H | m-D1 | H | m-D1 | A-14 |
| 53142 | m-D1 | H | m-D1 | H | m-D1 | A-21 |
| 53143 | m-D1 | H | m-D1 | H | m-D1 | A-27 |
| 53144 | m-D1 | H | m-D1 | H | m-D1 | A-30 |
| 53145 | m-D1 | H | m-D1 | H | m-D1 | A-31 |
| 53146 | m-D1 | H | m-D1 | H | m-D1 | A-34 |
| 53147 | m-D1 | H | m-D1 | H | m-D1 | A-38 |
| 53148 | m-D1 | H | m-D1 | H | m-D1 | A-39 |
| 53149 | m-D1 | H | m-D1 | H | m-D1 | A-40 |
| 53150 | m-D1 | H | m-D1 | H | m-D1 | A-43 |
| 53151 | m-D1 | H | m-D1 | H | m-D1 | A-44 |
| 53152 | m-D1 | H | m-D1 | H | m-D1 | A-45 |
| 53153 | m-D2 | H | m-D2 | H | m-D2 | A-6 |
| 53154 | m-D2 | H | m-D2 | H | m-D2 | A-7 |
| 53155 | m-D2 | H | m-D2 | H | m-D2 | A-10 |
| 53156 | m-D2 | H | m-D2 | H | m-D2 | A-12 |
| 53157 | m-D2 | H | m-D2 | H | m-D2 | A-14 |
| 53158 | m-D2 | H | m-D2 | H | m-D2 | A-21 |
| 53159 | m-D2 | H | m-D2 | H | m-D2 | A-27 |
| 53160 | m-D2 | H | m-D2 | H | m-D2 | A-30 |
| 53161 | m-D2 | H | m-D2 | H | m-D2 | A-31 |
| 53162 | m-D2 | H | m-D2 | H | m-D2 | A-34 |
| 53163 | m-D2 | H | m-D2 | H | m-D2 | A-38 |
| 53164 | m-D2 | H | m-D2 | H | m-D2 | A-39 |
| 53165 | m-D2 | H | m-D2 | H | m-D2 | A-40 |
| 53166 | m-D2 | H | m-D2 | H | m-D2 | A-43 |
| 53167 | m-D2 | H | m-D2 | H | m-D2 | A-44 |
| 53168 | m-D2 | H | m-D2 | H | m-D2 | A-45 |
| 53169 | m-D3 | H | m-D3 | H | m-D3 | A-6 |
| 53170 | m-D3 | H | m-D3 | H | m-D3 | A-7 |
| 53171 | m-D3 | H | m-D3 | H | m-D3 | A-10 |
| 53172 | m-D3 | H | m-D3 | H | m-D3 | A-12 |
| 53173 | m-D3 | H | m-D3 | H | m-D3 | A-14 |
| 53174 | m-D3 | H | m-D3 | H | m-D3 | A-21 |
| 53175 | m-D3 | H | m-D3 | H | m-D3 | A-27 |
| 53176 | m-D3 | H | m-D3 | H | m-D3 | A-30 |
| 53177 | m-D3 | H | m-D3 | H | m-D3 | A-31 |
| 53178 | m-D3 | H | m-D3 | H | m-D3 | A-34 |
| 53179 | m-D3 | H | m-D3 | H | m-D3 | A-38 |
| 53180 | m-D3 | H | m-D3 | H | m-D3 | A-39 |
| 53181 | m-D3 | H | m-D3 | H | m-D3 | A-40 |
| 53182 | m-D3 | H | m-D3 | H | m-D3 | A-43 |
| 53183 | m-D3 | H | m-D3 | H | m-D3 | A-44 |
| 53184 | m-D3 | H | m-D3 | H | m-D3 | A-45 |
| 53185 | m-D4 | H | m-D4 | H | m-D4 | A-6 |
| 53186 | m-D4 | H | m-D4 | H | m-D4 | A-7 |
| 53187 | m-D4 | H | m-D4 | H | m-D4 | A-10 |
| 53188 | m-D4 | H | m-D4 | H | m-D4 | A-12 |
| 53189 | m-D4 | H | m-D4 | H | m-D4 | A-14 |
| 53190 | m-D4 | H | m-D4 | H | m-D4 | A-21 |
| 53191 | m-D4 | H | m-D4 | H | m-D4 | A-27 |
| 53192 | m-D4 | H | m-D4 | H | m-D4 | A-30 |
| 53193 | m-D4 | H | m-D4 | H | m-D4 | A-31 |
| 53194 | m-D4 | H | m-D4 | H | m-D4 | A-34 |
| 53195 | m-D4 | H | m-D4 | H | m-D4 | A-38 |
| 53196 | m-D4 | H | m-D4 | H | m-D4 | A-39 |
| 53197 | m-D4 | H | m-D4 | H | m-D4 | A-40 |
| 53198 | m-D4 | H | m-D4 | H | m-D4 | A-43 |
| 53199 | m-D4 | H | m-D4 | H | m-D4 | A-44 |
| 53200 | m-D4 | H | m-D4 | H | m-D4 | A-45 |
| 53201 | m-D5 | H | m-D5 | H | m-D5 | A-6 |
| 53202 | m-D5 | H | m-D5 | H | m-D5 | A-7 |
| 53203 | m-D5 | H | m-D5 | H | m-D5 | A-10 |
| 53204 | m-D5 | H | m-D5 | H | m-D5 | A-12 |
| 53205 | m-D5 | H | m-D5 | H | m-D5 | A-14 |
| 53206 | m-D5 | H | m-D5 | H | m-D5 | A-21 |
| 53207 | m-D5 | H | m-D5 | H | m-D5 | A-27 |
| 53208 | m-D5 | H | m-D5 | H | m-D5 | A-30 |
| 53209 | m-D5 | H | m-D5 | H | m-D5 | A-31 |
| 53210 | m-D5 | H | m-D5 | H | m-D5 | A-34 |
| 53211 | m-D5 | H | m-D5 | H | m-D5 | A-38 |
| 53212 | m-D5 | H | m-D5 | H | m-D5 | A-39 |
| 53213 | m-D5 | H | m-D5 | H | m-D5 | A-40 |
| 53214 | m-D5 | H | m-D5 | H | m-D5 | A-43 |
| 53215 | m-D5 | H | m-D5 | H | m-D5 | A-44 |
| 53216 | m-D5 | H | m-D5 | H | m-D5 | A-45 |
| 53217 | m-D6 | H | m-D6 | H | m-D6 | A-6 |
| 53218 | m-D6 | H | m-D6 | H | m-D6 | A-7 |
| 53219 | m-D6 | H | m-D6 | H | m-D6 | A-10 |
| 53220 | m-D6 | H | m-D6 | H | m-D6 | A-12 |
| 53221 | m-D6 | H | m-D6 | H | m-D6 | A-14 |
| 53222 | m-D6 | H | m-D6 | H | m-D6 | A-21 |
| 53223 | m-D6 | H | m-D6 | H | m-D6 | A-27 |
| 53224 | m-D6 | H | m-D6 | H | m-D6 | A-30 |
| 53225 | m-D6 | H | m-D6 | H | m-D6 | A-31 |
| 53226 | m-D6 | H | m-D6 | H | m-D6 | A-34 |
| 53227 | m-D6 | H | m-D6 | H | m-D6 | A-38 |
| 53228 | m-D6 | H | m-D6 | H | m-D6 | A-39 |
| 53229 | m-D6 | H | m-D6 | H | m-D6 | A-40 |
| 53230 | m-D6 | H | m-D6 | H | m-D6 | A-43 |
| 53231 | m-D6 | H | m-D6 | H | m-D6 | A-44 |
| 53232 | m-D6 | H | m-D6 | H | m-D6 | A-45 |
| 53233 | m-D7 | H | m-D7 | H | m-D7 | A-6 |
| 53234 | m-D7 | H | m-D7 | H | m-D7 | A-7 |
| 53235 | m-D7 | H | m-D7 | H | m-D7 | A-10 |
| 53236 | m-D7 | H | m-D7 | H | m-D7 | A-12 |
| 53237 | m-D7 | H | m-D7 | H | m-D7 | A-14 |
| 53238 | m-D7 | H | m-D7 | H | m-D7 | A-21 |
| 53239 | m-D7 | H | m-D7 | H | m-D7 | A-27 |
| 53240 | m-D7 | H | m-D7 | H | m-D7 | A-30 |
| 53241 | m-D7 | H | m-D7 | H | m-D7 | A-31 |
| 53242 | m-D7 | H | m-D7 | H | m-D7 | A-34 |
| 53243 | m-D7 | H | m-D7 | H | m-D7 | A-38 |
| 53244 | m-D7 | H | m-D7 | H | m-D7 | A-39 |
| 53245 | m-D7 | H | m-D7 | H | m-D7 | A-40 |
| 53246 | m-D7 | H | m-D7 | H | m-D7 | A-43 |
| 53247 | m-D7 | H | m-D7 | H | m-D7 | A-44 |
| 53248 | m-D7 | H | m-D7 | H | m-D7 | A-45 |
| 53249 | m-D8 | H | m-D8 | H | m-D8 | A-6 |
| 53250 | m-D8 | H | m-D8 | H | m-D8 | A-7 |
| 53251 | m-D8 | H | m-D8 | H | m-D8 | A-10 |
| 53252 | m-D8 | H | m-D8 | H | m-D8 | A-12 |
| 53253 | m-D8 | H | m-D8 | H | m-D8 | A-14 |
| 53254 | m-D8 | H | m-D8 | H | m-D8 | A-21 |
| 53255 | m-D8 | H | m-D8 | H | m-D8 | A-27 |
| 53256 | m-D8 | H | m-D8 | H | m-D8 | A-30 |
| 53257 | m-D8 | H | m-D8 | H | m-D8 | A-31 |
| 53258 | m-D8 | H | m-D8 | H | m-D8 | A-34 |
| 53259 | m-D8 | H | m-D8 | H | m-D8 | A-38 |
| 53260 | m-D8 | H | m-D8 | H | m-D8 | A-39 |
| 53261 | m-D8 | H | m-D8 | H | m-D8 | A-40 |
| 53262 | m-D8 | H | m-D8 | H | m-D8 | A-43 |
| 53263 | m-D8 | H | m-D8 | H | m-D8 | A-44 |
| 53264 | m-D8 | H | m-D8 | H | m-D8 | A-45 |
| 53265 | m-D9 | H | m-D9 | H | m-D9 | A-6 |
| 53266 | m-D9 | H | m-D9 | H | m-D9 | A-7 |
| 53267 | m-D9 | H | m-D9 | H | m-D9 | A-10 |
| 53268 | m-D9 | H | m-D9 | H | m-D9 | A-12 |
| 53269 | m-D9 | H | m-D9 | H | m-D9 | A-14 |
| 53270 | m-D9 | H | m-D9 | H | m-D9 | A-21 |
| 53271 | m-D9 | H | m-D9 | H | m-D9 | A-27 |
| 53272 | m-D9 | H | m-D9 | H | m-D9 | A-30 |
| 53273 | m-D9 | H | m-D9 | H | m-D9 | A-31 |
| 53274 | m-D9 | H | m-D9 | H | m-D9 | A-34 |
| 53275 | m-D9 | H | m-D9 | H | m-D9 | A-38 |
| 53276 | m-D9 | H | m-D9 | H | m-D9 | A-39 |
| 53277 | m-D9 | H | m-D9 | H | m-D9 | A-40 |
| 53278 | m-D9 | H | m-D9 | H | m-D9 | A-43 |
| 53279 | m-D9 | H | m-D9 | H | m-D9 | A-44 |
| 53280 | m-D9 | H | m-D9 | H | m-D9 | A-45 |
| 53281 | m-D10 | H | m-D10 | H | m-D10 | A-6 |
| 53282 | m-D10 | H | m-D10 | H | m-D10 | A-7 |
| 53283 | m-D10 | H | m-D10 | H | m-D10 | A-10 |
| 53284 | m-D10 | H | m-D10 | H | m-D10 | A-12 |
| 53285 | m-D10 | H | m-D10 | H | m-D10 | A-14 |
| 53286 | m-D10 | H | m-D10 | H | m-D10 | A-21 |
| 53287 | m-D10 | H | m-D10 | H | m-D10 | A-27 |
| 53288 | m-D10 | H | m-D10 | H | m-D10 | A-30 |
| 53289 | m-D10 | H | m-D10 | H | m-D10 | A-31 |
| 53290 | m-D10 | H | m-D10 | H | m-D10 | A-34 |
| 53291 | m-D10 | H | m-D10 | H | m-D10 | A-38 |
| 53292 | m-D10 | H | m-D10 | H | m-D10 | A-39 |
| 53293 | m-D10 | H | m-D10 | H | m-D10 | A-40 |
| 53294 | m-D10 | H | m-D10 | H | m-D10 | A-43 |
| 53295 | m-D10 | H | m-D10 | H | m-D10 | A-44 |
| 53296 | m-D10 | H | m-D10 | H | m-D10 | A-45 |
| 53297 | m-D11 | H | m-D11 | H | m-D11 | A-6 |
| 53298 | m-D11 | H | m-D11 | H | m-D11 | A-7 |
| 53299 | m-D11 | H | m-D11 | H | m-D11 | A-10 |
| 53300 | m-D11 | H | m-D11 | H | m-D11 | A-12 |
| 53301 | m-D11 | H | m-D11 | H | m-D11 | A-14 |
| 53302 | m-D11 | H | m-D11 | H | m-D11 | A-21 |
| 53303 | m-D11 | H | m-D11 | H | m-D11 | A-27 |
| 53304 | m-D11 | H | m-D11 | H | m-D11 | A-30 |
| 53305 | m-D11 | H | m-D11 | H | m-D11 | A-31 |
| 53306 | m-D11 | H | m-D11 | H | m-D11 | A-34 |
| 53307 | m-D11 | H | m-D11 | H | m-D11 | A-38 |
| 53308 | m-D11 | H | m-D11 | H | m-D11 | A-39 |
| 53309 | m-D11 | H | m-D11 | H | m-D11 | A-40 |
| 53310 | m-D11 | H | m-D11 | H | m-D11 | A-43 |
| 53311 | m-D11 | H | m-D11 | H | m-D11 | A-44 |
| 53312 | m-D11 | H | m-D11 | H | m-D11 | A-45 |
| 53313 | m-D12 | H | m-D12 | H | m-D12 | A-6 |
| 53314 | m-D12 | H | m-D12 | H | m-D12 | A-7 |
| 53315 | m-D12 | H | m-D12 | H | m-D12 | A-10 |
| 53316 | m-D12 | H | m-D12 | H | m-D12 | A-12 |
| 53317 | m-D12 | H | m-D12 | H | m-D12 | A-14 |
| 53318 | m-D12 | H | m-D12 | H | m-D12 | A-21 |
| 53319 | m-D12 | H | m-D12 | H | m-D12 | A-27 |
| 53320 | m-D12 | H | m-D12 | H | m-D12 | A-30 |
| 53321 | m-D12 | H | m-D12 | H | m-D12 | A-31 |
| 53322 | m-D12 | H | m-D12 | H | m-D12 | A-34 |
| 53323 | m-D12 | H | m-D12 | H | m-D12 | A-38 |
| 53324 | m-D12 | H | m-D12 | H | m-D12 | A-39 |
| 53325 | m-D12 | H | m-D12 | H | m-D12 | A-40 |
| 53326 | m-D12 | H | m-D12 | H | m-D12 | A-43 |
| 53327 | m-D12 | H | m-D12 | H | m-D12 | A-44 |
| 53328 | m-D12 | H | m-D12 | H | m-D12 | A-45 |
| 53329 | m-D13 | H | m-D13 | H | m-D13 | A-6 |
| 53330 | m-D13 | H | m-D13 | H | m-D13 | A-7 |
| 53331 | m-D13 | H | m-D13 | H | m-D13 | A-10 |
| 53332 | m-D13 | H | m-D13 | H | m-D13 | A-12 |
| 53333 | m-D13 | H | m-D13 | H | m-D13 | A-14 |
| 53334 | m-D13 | H | m-D13 | H | m-D13 | A-21 |
| 53335 | m-D13 | H | m-D13 | H | m-D13 | A-27 |
| 53336 | m-D13 | H | m-D13 | H | m-D13 | A-30 |
| 53337 | m-D13 | H | m-D13 | H | m-D13 | A-31 |
| 53338 | m-D13 | H | m-D13 | H | m-D13 | A-34 |
| 53339 | m-D13 | H | m-D13 | H | m-D13 | A-38 |
| 53340 | m-D13 | H | m-D13 | H | m-D13 | A-39 |
| 53341 | m-D13 | H | m-D13 | H | m-D13 | A-40 |
| 53342 | m-D13 | H | m-D13 | H | m-D13 | A-43 |
| 53343 | m-D13 | H | m-D13 | H | m-D13 | A-44 |
| 53344 | m-D13 | H | m-D13 | H | m-D13 | A-45 |
| 53345 | m-D14 | H | m-D14 | H | m-D14 | A-6 |
| 53346 | m-D14 | H | m-D14 | H | m-D14 | A-7 |
| 53347 | m-D14 | H | m-D14 | H | m-D14 | A-10 |
| 53348 | m-D14 | H | m-D14 | H | m-D14 | A-12 |
| 53349 | m-D14 | H | m-D14 | H | m-D14 | A-14 |
| 53350 | m-D14 | H | m-D14 | H | m-D14 | A-21 |
| 53351 | m-D14 | H | m-D14 | H | m-D14 | A-27 |
| 53352 | m-D14 | H | m-D14 | H | m-D14 | A-30 |
| 53353 | m-D14 | H | m-D14 | H | m-D14 | A-31 |
| 53354 | m-D14 | H | m-D14 | H | m-D14 | A-34 |
| 53355 | m-D14 | H | m-D14 | H | m-D14 | A-38 |
| 53356 | m-D14 | H | m-D14 | H | m-D14 | A-39 |
| 53357 | m-D14 | H | m-D14 | H | m-D14 | A-40 |
| 53358 | m-D14 | H | m-D14 | H | m-D14 | A-43 |
| 53359 | m-D14 | H | m-D14 | H | m-D14 | A-44 |
| 53360 | m-D14 | H | m-D14 | H | m-D14 | A-45 |
| 53361 | m-D1 | m-D1 | m-D1 | m-D1 | H | A-6 |
| 53362 | m-D1 | m-D1 | m-D1 | m-D1 | H | A-7 |
| 53363 | m-D1 | m-D1 | m-D1 | m-D1 | H | A-10 |
| 53364 | m-D1 | m-D1 | m-D1 | m-D1 | H | A-12 |
| 53365 | m-D1 | m-D1 | m-D1 | m-D1 | H | A-14 |
| 53366 | m-D1 | m-D1 | m-D1 | m-D1 | H | A-21 |
| 53367 | m-D1 | m-D1 | m-D1 | m-D1 | H | A-27 |
| 53368 | m-D1 | m-D1 | m-D1 | m-D1 | H | A-30 |
| 53369 | m-D1 | m-D1 | m-D1 | m-D1 | H | A-31 |
| 53370 | m-D1 | m-D1 | m-D1 | m-D1 | H | A-34 |
| 53371 | m-D1 | m-D1 | m-D1 | m-D1 | H | A-38 |
| 53372 | m-D1 | m-D1 | m-D1 | m-D1 | H | A-39 |
| 53373 | m-D1 | m-D1 | m-D1 | m-D1 | H | A-40 |
| 53374 | m-D1 | m-D1 | m-D1 | m-D1 | H | A-43 |
| 53375 | m-D1 | m-D1 | m-D1 | m-D1 | H | A-44 |
| 53376 | m-D1 | m-D1 | m-D1 | m-D1 | H | A-45 |
| 53377 | m-D2 | m-D2 | m-D2 | m-D2 | H | A-6 |
| 53378 | m-D2 | m-D2 | m-D2 | m-D2 | H | A-7 |
| 53379 | m-D2 | m-D2 | m-D2 | m-D2 | H | A-10 |
| 53380 | m-D2 | m-D2 | m-D2 | m-D2 | H | A-12 |
| 53381 | m-D2 | m-D2 | m-D2 | m-D2 | H | A-14 |
| 53382 | m-D2 | m-D2 | m-D2 | m-D2 | H | A-21 |
| 53383 | m-D2 | m-D2 | m-D2 | m-D2 | H | A-27 |
| 53384 | m-D2 | m-D2 | m-D2 | m-D2 | H | A-30 |
| 53385 | m-D2 | m-D2 | m-D2 | m-D2 | H | A-31 |
| 53386 | m-D2 | m-D2 | m-D2 | m-D2 | H | A-34 |
| 53387 | m-D2 | m-D2 | m-D2 | m-D2 | H | A-38 |
| 53388 | m-D2 | m-D2 | m-D2 | m-D2 | H | A-39 |
| 53389 | m-D2 | m-D2 | m-D2 | m-D2 | H | A-40 |
| 53390 | m-D2 | m-D2 | m-D2 | m-D2 | H | A-43 |
| 53391 | m-D2 | m-D2 | m-D2 | m-D2 | H | A-44 |
| 53392 | m-D2 | m-D2 | m-D2 | m-D2 | H | A-45 |
| 53393 | m-D3 | m-D3 | m-D3 | m-D3 | H | A-6 |
| 53394 | m-D3 | m-D3 | m-D3 | m-D3 | H | A-7 |
| 53395 | m-D3 | m-D3 | m-D3 | m-D3 | H | A-10 |
| 53396 | m-D3 | m-D3 | m-D3 | m-D3 | H | A-12 |
| 53397 | m-D3 | m-D3 | m-D3 | m-D3 | H | A-14 |
| 53398 | m-D3 | m-D3 | m-D3 | m-D3 | H | A-21 |
| 53399 | m-D3 | m-D3 | m-D3 | m-D3 | H | A-27 |
| 53400 | m-D3 | m-D3 | m-D3 | m-D3 | H | A-30 |
| 53401 | m-D3 | m-D3 | m-D3 | m-D3 | H | A-31 |
| 53402 | m-D3 | m-D3 | m-D3 | m-D3 | H | A-34 |
| 53403 | m-D3 | m-D3 | m-D3 | m-D3 | H | A-38 |
| 53404 | m-D3 | m-D3 | m-D3 | m-D3 | H | A-39 |
| 53405 | m-D3 | m-D3 | m-D3 | m-D3 | H | A-40 |
| 53406 | m-D3 | m-D3 | m-D3 | m-D3 | H | A-43 |
| 53407 | m-D3 | m-D3 | m-D3 | m-D3 | H | A-44 |
| 53408 | m-D3 | m-D3 | m-D3 | m-D3 | H | A-45 |
| 53409 | m-D4 | m-D4 | m-D4 | m-D4 | H | A-6 |
| 53410 | m-D4 | m-D4 | m-D4 | m-D4 | H | A-7 |
| 53411 | m-D4 | m-D4 | m-D4 | m-D4 | H | A-10 |
| 53412 | m-D4 | m-D4 | m-D4 | m-D4 | H | A-12 |
| 53413 | m-D4 | m-D4 | m-D4 | m-D4 | H | A-14 |
| 53414 | m-D4 | m-D4 | m-D4 | m-D4 | H | A-21 |
| 53415 | m-D4 | m-D4 | m-D4 | m-D4 | H | A-27 |
| 53416 | m-D4 | m-D4 | m-D4 | m-D4 | H | A-30 |
| 53417 | m-D4 | m-D4 | m-D4 | m-D4 | H | A-31 |
| 53418 | m-D4 | m-D4 | m-D4 | m-D4 | H | A-34 |
| 53419 | m-D4 | m-D4 | m-D4 | m-D4 | H | A-38 |
| 53420 | m-D4 | m-D4 | m-D4 | m-D4 | H | A-39 |
| 53421 | m-D4 | m-D4 | m-D4 | m-D4 | H | A-40 |
| 53422 | m-D4 | m-D4 | m-D4 | m-D4 | H | A-43 |
| 53423 | m-D4 | m-D4 | m-D4 | m-D4 | H | A-44 |
| 53424 | m-D4 | m-D4 | m-D4 | m-D4 | H | A-45 |
| 53425 | m-D5 | m-D5 | m-D5 | m-D5 | H | A-6 |
| 53426 | m-D5 | m-D5 | m-D5 | m-D5 | H | A-7 |
| 53427 | m-D5 | m-D5 | m-D5 | m-D5 | H | A-10 |
| 53428 | m-D5 | m-D5 | m-D5 | m-D5 | H | A-12 |
| 53429 | m-D5 | m-D5 | m-D5 | m-D5 | H | A-14 |
| 53430 | m-D5 | m-D5 | m-D5 | m-D5 | H | A-21 |
| 53431 | m-D5 | m-D5 | m-D5 | m-D5 | H | A-27 |
| 53432 | m-D5 | m-D5 | m-D5 | m-D5 | H | A-30 |
| 53433 | m-D5 | m-D5 | m-D5 | m-D5 | H | A-31 |
| 53434 | m-D5 | m-D5 | m-D5 | m-D5 | H | A-34 |
| 53435 | m-D5 | m-D5 | m-D5 | m-D5 | H | A-38 |
| 53436 | m-D5 | m-D5 | m-D5 | m-D5 | H | A-39 |
| 53437 | m-D5 | m-D5 | m-D5 | m-D5 | H | A-40 |
| 53438 | m-D5 | m-D5 | m-D5 | m-D5 | H | A-43 |
| 53439 | m-D5 | m-D5 | m-D5 | m-D5 | H | A-44 |
| 53440 | m-D5 | m-D5 | m-D5 | m-D5 | H | A-45 |
| 53441 | m-D6 | m-D6 | m-D6 | m-D6 | H | A-6 |
| 53442 | m-D6 | m-D6 | m-D6 | m-D6 | H | A-7 |
| 53443 | m-D6 | m-D6 | m-D6 | m-D6 | H | A-10 |
| 53444 | m-D6 | m-D6 | m-D6 | m-D6 | H | A-12 |
| 53445 | m-D6 | m-D6 | m-D6 | m-D6 | H | A-14 |
| 53446 | m-D6 | m-D6 | m-D6 | m-D6 | H | A-21 |
| 53447 | m-D6 | m-D6 | m-D6 | m-D6 | H | A-27 |
| 53448 | m-D6 | m-D6 | m-D6 | m-D6 | H | A-30 |
| 53449 | m-D6 | m-D6 | m-D6 | m-D6 | H | A-31 |
| 53450 | m-D6 | m-D6 | m-D6 | m-D6 | H | A-34 |
| 53451 | m-D6 | m-D6 | m-D6 | m-D6 | H | A-38 |
| 53452 | m-D6 | m-D6 | m-D6 | m-D6 | H | A-39 |
| 53453 | m-D6 | m-D6 | m-D6 | m-D6 | H | A-40 |
| 53454 | m-D6 | m-D6 | m-D6 | m-D6 | H | A-43 |
| 53455 | m-D6 | m-D6 | m-D6 | m-D6 | H | A-44 |
| 53456 | m-D6 | m-D6 | m-D6 | m-D6 | H | A-45 |
| 53457 | m-D7 | m-D7 | m-D7 | m-D7 | H | A-6 |
| 53458 | m-D7 | m-D7 | m-D7 | m-D7 | H | A-7 |
| 53459 | m-D7 | m-D7 | m-D7 | m-D7 | H | A-10 |
| 53460 | m-D7 | m-D7 | m-D7 | m-D7 | H | A-12 |
| 53461 | m-D7 | m-D7 | m-D7 | m-D7 | H | A-14 |
| 53462 | m-D7 | m-D7 | m-D7 | m-D7 | H | A-21 |
| 53463 | m-D7 | m-D7 | m-D7 | m-D7 | H | A-27 |
| 53464 | m-D7 | m-D7 | m-D7 | m-D7 | H | A-30 |
| 53465 | m-D7 | m-D7 | m-D7 | m-D7 | H | A-31 |
| 53466 | m-D7 | m-D7 | m-D7 | m-D7 | H | A-34 |
| 53467 | m-D7 | m-D7 | m-D7 | m-D7 | H | A-38 |
| 53468 | m-D7 | m-D7 | m-D7 | m-D7 | H | A-39 |
| 53469 | m-D7 | m-D7 | m-D7 | m-D7 | H | A-40 |
| 53470 | m-D7 | m-D7 | m-D7 | m-D7 | H | A-43 |
| 53471 | m-D7 | m-D7 | m-D7 | m-D7 | H | A-44 |
| 53472 | m-D7 | m-D7 | m-D7 | m-D7 | H | A-45 |
| 53473 | m-D8 | m-D8 | m-D8 | m-D8 | H | A-6 |
| 53474 | m-D8 | m-D8 | m-D8 | m-D8 | H | A-7 |
| 53475 | m-D8 | m-D8 | m-D8 | m-D8 | H | A-10 |
| 53476 | m-D8 | m-D8 | m-D8 | m-D8 | H | A-12 |
| 53477 | m-D8 | m-D8 | m-D8 | m-D8 | H | A-14 |
| 53478 | m-D8 | m-D8 | m-D8 | m-D8 | H | A-21 |
| 53479 | m-D8 | m-D8 | m-D8 | m-D8 | H | A-27 |
| 53480 | m-D8 | m-D8 | m-D8 | m-D8 | H | A-30 |
| 53481 | m-D8 | m-D8 | m-D8 | m-D8 | H | A-31 |
| 53482 | m-D8 | m-D8 | m-D8 | m-D8 | H | A-34 |
| 53483 | m-D8 | m-D8 | m-D8 | m-D8 | H | A-38 |
| 53484 | m-D8 | m-D8 | m-D8 | m-D8 | H | A-39 |
| 53485 | m-D8 | m-D8 | m-D8 | m-D8 | H | A-40 |
| 53486 | m-D8 | m-D8 | m-D8 | m-D8 | H | A-43 |
| 53487 | m-D8 | m-D8 | m-D8 | m-D8 | H | A-44 |
| 53488 | m-D8 | m-D8 | m-D8 | m-D8 | H | A-45 |
| 53489 | m-D9 | m-D9 | m-D9 | m-D9 | H | A-6 |
| 53490 | m-D9 | m-D9 | m-D9 | m-D9 | H | A-7 |
| 53491 | m-D9 | m-D9 | m-D9 | m-D9 | H | A-10 |
| 53492 | m-D9 | m-D9 | m-D9 | m-D9 | H | A-12 |
| 53493 | m-D9 | m-D9 | m-D9 | m-D9 | H | A-14 |
| 53494 | m-D9 | m-D9 | m-D9 | m-D9 | H | A-21 |
| 53495 | m-D9 | m-D9 | m-D9 | m-D9 | H | A-27 |
| 53496 | m-D9 | m-D9 | m-D9 | m-D9 | H | A-30 |
| 53497 | m-D9 | m-D9 | m-D9 | m-D9 | H | A-31 |
| 53498 | m-D9 | m-D9 | m-D9 | m-D9 | H | A-34 |
| 53499 | m-D9 | m-D9 | m-D9 | m-D9 | H | A-38 |
| 53500 | m-D9 | m-D9 | m-D9 | m-D9 | H | A-39 |
| 53501 | m-D9 | m-D9 | m-D9 | m-D9 | H | A-40 |
| 53502 | m-D9 | m-D9 | m-D9 | m-D9 | H | A-43 |
| 53503 | m-D9 | m-D9 | m-D9 | m-D9 | H | A-44 |
| 53504 | m-D9 | m-D9 | m-D9 | m-D9 | H | A-45 |
| 53505 | m-D10 | m-D10 | m-D10 | m-D10 | H | A-6 |
| 53506 | m-D10 | m-D10 | m-D10 | m-D10 | H | A-7 |
| 53507 | m-D10 | m-D10 | m-D10 | m-D10 | H | A-10 |
| 53508 | m-D10 | m-D10 | m-D10 | m-D10 | H | A-12 |
| 53509 | m-D10 | m-D10 | m-D10 | m-D10 | H | A-14 |
| 53510 | m-D10 | m-D10 | m-D10 | m-D10 | H | A-21 |
| 53511 | m-D10 | m-D10 | m-D10 | m-D10 | H | A-27 |
| 53512 | m-D10 | m-D10 | m-D10 | m-D10 | H | A-30 |
| 53513 | m-D10 | m-D10 | m-D10 | m-D10 | H | A-31 |
| 53514 | m-D10 | m-D10 | m-D10 | m-D10 | H | A-34 |
| 53515 | m-D10 | m-D10 | m-D10 | m-D10 | H | A-38 |
| 53516 | m-D10 | m-D10 | m-D10 | m-D10 | H | A-39 |
| 53517 | m-D10 | m-D10 | m-D10 | m-D10 | H | A-40 |
| 53518 | m-D10 | m-D10 | m-D10 | m-D10 | H | A-43 |
| 53519 | m-D10 | m-D10 | m-D10 | m-D10 | H | A-44 |
| 53520 | m-D10 | m-D10 | m-D10 | m-D10 | H | A-45 |
| 53521 | m-D11 | m-D11 | m-D11 | m-D11 | H | A-6 |
| 53522 | m-D11 | m-D11 | m-D11 | m-D11 | H | A-7 |
| 53523 | m-D11 | m-D11 | m-D11 | m-D11 | H | A-10 |
| 53524 | m-D11 | m-D11 | m-D11 | m-D11 | H | A-12 |
| 53525 | m-D11 | m-D11 | m-D11 | m-D11 | H | A-14 |
| 53526 | m-D11 | m-D11 | m-D11 | m-D11 | H | A-21 |
| 53527 | m-D11 | m-D11 | m-D11 | m-D11 | H | A-27 |
| 53528 | m-D11 | m-D11 | m-D11 | m-D11 | H | A-30 |
| 53529 | m-D11 | m-D11 | m-D11 | m-D11 | H | A-31 |
| 53530 | m-D11 | m-D11 | m-D11 | m-D11 | H | A-34 |
| 53531 | m-D11 | m-D11 | m-D11 | m-D11 | H | A-38 |
| 53532 | m-D11 | m-D11 | m-D11 | m-D11 | H | A-39 |
| 53533 | m-D11 | m-D11 | m-D11 | m-D11 | H | A-40 |
| 53534 | m-D11 | m-D11 | m-D11 | m-D11 | H | A-43 |
| 53535 | m-D11 | m-D11 | m-D11 | m-D11 | H | A-44 |
| 53536 | m-D11 | m-D11 | m-D11 | m-D11 | H | A-45 |
| 53537 | m-D12 | m-D12 | m-D12 | m-D12 | H | A-6 |
| 53538 | m-D12 | m-D12 | m-D12 | m-D12 | H | A-7 |
| 53539 | m-D12 | m-D12 | m-D12 | m-D12 | H | A-10 |
| 53540 | m-D12 | m-D12 | m-D12 | m-D12 | H | A-12 |
| 53541 | m-D12 | m-D12 | m-D12 | m-D12 | H | A-14 |
| 53542 | m-D12 | m-D12 | m-D12 | m-D12 | H | A-21 |
| 53543 | m-D12 | m-D12 | m-D12 | m-D12 | H | A-27 |
| 53544 | m-D12 | m-D12 | m-D12 | m-D12 | H | A-30 |
| 53545 | m-D12 | m-D12 | m-D12 | m-D12 | H | A-31 |
| 53546 | m-D12 | m-D12 | m-D12 | m-D12 | H | A-34 |
| 53547 | m-D12 | m-D12 | m-D12 | m-D12 | H | A-38 |
| 53548 | m-D12 | m-D12 | m-D12 | m-D12 | H | A-39 |
| 53549 | m-D12 | m-D12 | m-D12 | m-D12 | H | A-40 |
| 53550 | m-D12 | m-D12 | m-D12 | m-D12 | H | A-43 |
| 53551 | m-D12 | m-D12 | m-D12 | m-D12 | H | A-44 |
| 53552 | m-D12 | m-D12 | m-D12 | m-D12 | H | A-45 |
| 53553 | m-D13 | m-D13 | m-D13 | m-D13 | H | A-6 |
| 53554 | m-D13 | m-D13 | m-D13 | m-D13 | H | A-7 |
| 53555 | m-D13 | m-D13 | m-D13 | m-D13 | H | A-10 |
| 53556 | m-D13 | m-D13 | m-D13 | m-D13 | H | A-12 |
| 53557 | m-D13 | m-D13 | m-D13 | m-D13 | H | A-14 |
| 53558 | m-D13 | m-D13 | m-D13 | m-D13 | H | A-21 |
| 53559 | m-D13 | m-D13 | m-D13 | m-D13 | H | A-27 |
| 53560 | m-D13 | m-D13 | m-D13 | m-D13 | H | A-30 |
| 53561 | m-D13 | m-D13 | m-D13 | m-D13 | H | A-31 |
| 53562 | m-D13 | m-D13 | m-D13 | m-D13 | H | A-34 |
| 53563 | m-D13 | m-D13 | m-D13 | m-D13 | H | A-38 |
| 53564 | m-D13 | m-D13 | m-D13 | m-D13 | H | A-39 |
| 53565 | m-D13 | m-D13 | m-D13 | m-D13 | H | A-40 |
| 53566 | m-D13 | m-D13 | m-D13 | m-D13 | H | A-43 |
| 53567 | m-D13 | m-D13 | m-D13 | m-D13 | H | A-44 |
| 53568 | m-D13 | m-D13 | m-D13 | m-D13 | H | A-45 |
| 53569 | m-D14 | m-D14 | m-D14 | m-D14 | H | A-6 |
| 53570 | m-D14 | m-D14 | m-D14 | m-D14 | H | A-7 |
| 53571 | m-D14 | m-D14 | m-D14 | m-D14 | H | A-10 |
| 53572 | m-D14 | m-D14 | m-D14 | m-D14 | H | A-12 |
| 53573 | m-D14 | m-D14 | m-D14 | m-D14 | H | A-14 |
| 53574 | m-D14 | m-D14 | m-D14 | m-D14 | H | A-21 |
| 53575 | m-D14 | m-D14 | m-D14 | m-D14 | H | A-27 |
| 53576 | m-D14 | m-D14 | m-D14 | m-D14 | H | A-30 |
| 53577 | m-D14 | m-D14 | m-D14 | m-D14 | H | A-31 |
| 53578 | m-D14 | m-D14 | m-D14 | m-D14 | H | A-34 |
| 53579 | m-D14 | m-D14 | m-D14 | m-D14 | H | A-38 |
| 53580 | m-D14 | m-D14 | m-D14 | m-D14 | H | A-39 |
| 53581 | m-D14 | m-D14 | m-D14 | m-D14 | H | A-40 |
| 53582 | m-D14 | m-D14 | m-D14 | m-D14 | H | A-43 |
| 53583 | m-D14 | m-D14 | m-D14 | m-D14 | H | A-44 |
| 53584 | m-D14 | m-D14 | m-D14 | m-D14 | H | A-45 |
| 53585 | m-D1 | m-D1 | m-D1 | H | m-D1 | A-6 |
| 53586 | m-D1 | m-D1 | m-D1 | H | m-D1 | A-7 |
| 53587 | m-D1 | m-D1 | m-D1 | H | m-D1 | A-10 |
| 53588 | m-D1 | m-D1 | m-D1 | H | m-D1 | A-12 |
| 53589 | m-D1 | m-D1 | m-D1 | H | m-D1 | A-14 |
| 53590 | m-D1 | m-D1 | m-D1 | H | m-D1 | A-21 |
| 53591 | m-D1 | m-D1 | m-D1 | H | m-D1 | A-27 |
| 53592 | m-D1 | m-D1 | m-D1 | H | m-D1 | A-30 |
| 53593 | m-D1 | m-D1 | m-D1 | H | m-D1 | A-31 |
| 53594 | m-D1 | m-D1 | m-D1 | H | m-D1 | A-34 |
| 53595 | m-D1 | m-D1 | m-D1 | H | m-D1 | A-38 |
| 53596 | m-D1 | m-D1 | m-D1 | H | m-D1 | A-39 |
| 53597 | m-D1 | m-D1 | m-D1 | H | m-D1 | A-40 |
| 53598 | m-D1 | m-D1 | m-D1 | H | m-D1 | A-43 |
| 53599 | m-D1 | m-D1 | m-D1 | H | m-D1 | A-44 |
| 53600 | m-D1 | m-D1 | m-D1 | H | m-D1 | A-45 |
| 53601 | m-D2 | m-D2 | m-D2 | H | m-D2 | A-6 |
| 53602 | m-D2 | m-D2 | m-D2 | H | m-D2 | A-7 |
| 53603 | m-D2 | m-D2 | m-D2 | H | m-D2 | A-10 |
| 53604 | m-D2 | m-D2 | m-D2 | H | m-D2 | A-12 |
| 53605 | m-D2 | m-D2 | m-D2 | H | m-D2 | A-14 |
| 53606 | m-D2 | m-D2 | m-D2 | H | m-D2 | A-21 |
| 53607 | m-D2 | m-D2 | m-D2 | H | m-D2 | A-27 |
| 53608 | m-D2 | m-D2 | m-D2 | H | m-D2 | A-30 |
| 53609 | m-D2 | m-D2 | m-D2 | H | m-D2 | A-31 |
| 53610 | m-D2 | m-D2 | m-D2 | H | m-D2 | A-34 |
| 53611 | m-D2 | m-D2 | m-D2 | H | m-D2 | A-38 |
| 53612 | m-D2 | m-D2 | m-D2 | H | m-D2 | A-39 |
| 53613 | m-D2 | m-D2 | m-D2 | H | m-D2 | A-40 |
| 53614 | m-D2 | m-D2 | m-D2 | H | m-D2 | A-43 |
| 53615 | m-D2 | m-D2 | m-D2 | H | m-D2 | A-44 |
| 53616 | m-D2 | m-D2 | m-D2 | H | m-D2 | A-45 |
| 53617 | m-D3 | m-D3 | m-D3 | H | m-D3 | A-6 |
| 53618 | m-D3 | m-D3 | m-D3 | H | m-D3 | A-7 |
| 53619 | m-D3 | m-D3 | m-D3 | H | m-D3 | A-10 |
| 53620 | m-D3 | m-D3 | m-D3 | H | m-D3 | A-12 |
| 53621 | m-D3 | m-D3 | m-D3 | H | m-D3 | A-14 |
| 53622 | m-D3 | m-D3 | m-D3 | H | m-D3 | A-21 |
| 53623 | m-D3 | m-D3 | m-D3 | H | m-D3 | A-27 |
| 53624 | m-D3 | m-D3 | m-D3 | H | m-D3 | A-30 |
| 53625 | m-D3 | m-D3 | m-D3 | H | m-D3 | A-31 |
| 53626 | m-D3 | m-D3 | m-D3 | H | m-D3 | A-34 |
| 53627 | m-D3 | m-D3 | m-D3 | H | m-D3 | A-38 |
| 53628 | m-D3 | m-D3 | m-D3 | H | m-D3 | A-39 |
| 53629 | m-D3 | m-D3 | m-D3 | H | m-D3 | A-40 |
| 53630 | m-D3 | m-D3 | m-D3 | H | m-D3 | A-43 |
| 53631 | m-D3 | m-D3 | m-D3 | H | m-D3 | A-44 |
| 53632 | m-D3 | m-D3 | m-D3 | H | m-D3 | A-45 |
| 53633 | m-D4 | m-D4 | m-D4 | H | m-D4 | A-6 |
| 53634 | m-D4 | m-D4 | m-D4 | H | m-D4 | A-7 |
| 53635 | m-D4 | m-D4 | m-D4 | H | m-D4 | A-10 |
| 53636 | m-D4 | m-D4 | m-D4 | H | m-D4 | A-12 |
| 53637 | m-D4 | m-D4 | m-D4 | H | m-D4 | A-14 |
| 53638 | m-D4 | m-D4 | m-D4 | H | m-D4 | A-21 |
| 53639 | m-D4 | m-D4 | m-D4 | H | m-D4 | A-27 |
| 53640 | m-D4 | m-D4 | m-D4 | H | m-D4 | A-30 |
| 53641 | m-D4 | m-D4 | m-D4 | H | m-D4 | A-31 |
| 53642 | m-D4 | m-D4 | m-D4 | H | m-D4 | A-34 |
| 53643 | m-D4 | m-D4 | m-D4 | H | m-D4 | A-38 |
| 53644 | m-D4 | m-D4 | m-D4 | H | m-D4 | A-39 |
| 53645 | m-D4 | m-D4 | m-D4 | H | m-D4 | A-40 |
| 53646 | m-D4 | m-D4 | m-D4 | H | m-D4 | A-43 |
| 53647 | m-D4 | m-D4 | m-D4 | H | m-D4 | A-44 |
| 53648 | m-D4 | m-D4 | m-D4 | H | m-D4 | A-45 |
| 53649 | m-D5 | m-D5 | m-D5 | H | m-D5 | A-6 |
| 53650 | m-D5 | m-D5 | m-D5 | H | m-D5 | A-7 |
| 53651 | m-D5 | m-D5 | m-D5 | H | m-D5 | A-10 |
| 53652 | m-D5 | m-D5 | m-D5 | H | m-D5 | A-12 |
| 53653 | m-D5 | m-D5 | m-D5 | H | m-D5 | A-14 |
| 53654 | m-D5 | m-D5 | m-D5 | H | m-D5 | A-21 |
| 53655 | m-D5 | m-D5 | m-D5 | H | m-D5 | A-27 |
| 53656 | m-D5 | m-D5 | m-D5 | H | m-D5 | A-30 |
| 53657 | m-D5 | m-D5 | m-D5 | H | m-D5 | A-31 |
| 53658 | m-D5 | m-D5 | m-D5 | H | m-D5 | A-34 |
| 53659 | m-D5 | m-D5 | m-D5 | H | m-D5 | A-38 |
| 53660 | m-D5 | m-D5 | m-D5 | H | m-D5 | A-39 |
| 53661 | m-D5 | m-D5 | m-D5 | H | m-D5 | A-40 |
| 53662 | m-D5 | m-D5 | m-D5 | H | m-D5 | A-43 |
| 53663 | m-D5 | m-D5 | m-D5 | H | m-D5 | A-44 |
| 53664 | m-D5 | m-D5 | m-D5 | H | m-D5 | A-45 |
| 53665 | m-D6 | m-D6 | m-D6 | H | m-D6 | A-6 |
| 53666 | m-D6 | m-D6 | m-D6 | H | m-D6 | A-7 |
| 53667 | m-D6 | m-D6 | m-D6 | H | m-D6 | A-10 |
| 53668 | m-D6 | m-D6 | m-D6 | H | m-D6 | A-12 |
| 53669 | m-D6 | m-D6 | m-D6 | H | m-D6 | A-14 |
| 53670 | m-D6 | m-D6 | m-D6 | H | m-D6 | A-21 |
| 53671 | m-D6 | m-D6 | m-D6 | H | m-D6 | A-27 |
| 53672 | m-D6 | m-D6 | m-D6 | H | m-D6 | A-30 |
| 53673 | m-D6 | m-D6 | m-D6 | H | m-D6 | A-31 |
| 53674 | m-D6 | m-D6 | m-D6 | H | m-D6 | A-34 |
| 53675 | m-D6 | m-D6 | m-D6 | H | m-D6 | A-38 |
| 53676 | m-D6 | m-D6 | m-D6 | H | m-D6 | A-39 |
| 53677 | m-D6 | m-D6 | m-D6 | H | m-D6 | A-40 |
| 53678 | m-D6 | m-D6 | m-D6 | H | m-D6 | A-43 |
| 53679 | m-D6 | m-D6 | m-D6 | H | m-D6 | A-44 |
| 53680 | m-D6 | m-D6 | m-D6 | H | m-D6 | A-45 |
| 53681 | m-D7 | m-D7 | m-D7 | H | m-D7 | A-6 |
| 53682 | m-D7 | m-D7 | m-D7 | H | m-D7 | A-7 |
| 53683 | m-D7 | m-D7 | m-D7 | H | m-D7 | A-10 |
| 53684 | m-D7 | m-D7 | m-D7 | H | m-D7 | A-12 |
| 53685 | m-D7 | m-D7 | m-D7 | H | m-D7 | A-14 |
| 53686 | m-D7 | m-D7 | m-D7 | H | m-D7 | A-21 |
| 53687 | m-D7 | m-D7 | m-D7 | H | m-D7 | A-27 |
| 53688 | m-D7 | m-D7 | m-D7 | H | m-D7 | A-30 |
| 53689 | m-D7 | m-D7 | m-D7 | H | m-D7 | A-31 |
| 53690 | m-D7 | m-D7 | m-D7 | H | m-D7 | A-34 |
| 53691 | m-D7 | m-D7 | m-D7 | H | m-D7 | A-38 |
| 53692 | m-D7 | m-D7 | m-D7 | H | m-D7 | A-39 |
| 53693 | m-D7 | m-D7 | m-D7 | H | m-D7 | A-40 |
| 53694 | m-D7 | m-D7 | m-D7 | H | m-D7 | A-43 |
| 53695 | m-D7 | m-D7 | m-D7 | H | m-D7 | A-44 |
| 53696 | m-D7 | m-D7 | m-D7 | H | m-D7 | A-45 |
| 53697 | m-D8 | m-D8 | m-D8 | H | m-D8 | A-6 |
| 53698 | m-D8 | m-D8 | m-D8 | H | m-D8 | A-7 |
| 53699 | m-D8 | m-D8 | m-D8 | H | m-D8 | A-10 |
| 53700 | m-D8 | m-D8 | m-D8 | H | m-D8 | A-12 |
| 53701 | m-D8 | m-D8 | m-D8 | H | m-D8 | A-14 |
| 53702 | m-D8 | m-D8 | m-D8 | H | m-D8 | A-21 |
| 53703 | m-D8 | m-D8 | m-D8 | H | m-D8 | A-27 |
| 53704 | m-D8 | m-D8 | m-D8 | H | m-D8 | A-30 |
| 53705 | m-D8 | m-D8 | m-D8 | H | m-D8 | A-31 |
| 53706 | m-D8 | m-D8 | m-D8 | H | m-D8 | A-34 |
| 53707 | m-D8 | m-D8 | m-D8 | H | m-D8 | A-38 |
| 53708 | m-D8 | m-D8 | m-D8 | H | m-D8 | A-39 |
| 53709 | m-D8 | m-D8 | m-D8 | H | m-D8 | A-40 |
| 53710 | m-D8 | m-D8 | m-D8 | H | m-D8 | A-43 |
| 53711 | m-D8 | m-D8 | m-D8 | H | m-D8 | A-44 |
| 53712 | m-D8 | m-D8 | m-D8 | H | m-D8 | A-45 |
| 53713 | m-D9 | m-D9 | m-D9 | H | m-D9 | A-6 |
| 53714 | m-D9 | m-D9 | m-D9 | H | m-D9 | A-7 |
| 53715 | m-D9 | m-D9 | m-D9 | H | m-D9 | A-10 |
| 53716 | m-D9 | m-D9 | m-D9 | H | m-D9 | A-12 |
| 53717 | m-D9 | m-D9 | m-D9 | H | m-D9 | A-14 |
| 53718 | m-D9 | m-D9 | m-D9 | H | m-D9 | A-21 |
| 53719 | m-D9 | m-D9 | m-D9 | H | m-D9 | A-27 |
| 53720 | m-D9 | m-D9 | m-D9 | H | m-D9 | A-30 |
| 53721 | m-D9 | m-D9 | m-D9 | H | m-D9 | A-31 |
| 53722 | m-D9 | m-D9 | m-D9 | H | m-D9 | A-34 |
| 53723 | m-D9 | m-D9 | m-D9 | H | m-D9 | A-38 |
| 53724 | m-D9 | m-D9 | m-D9 | H | m-D9 | A-39 |
| 53725 | m-D9 | m-D9 | m-D9 | H | m-D9 | A-40 |
| 53726 | m-D9 | m-D9 | m-D9 | H | m-D9 | A-43 |
| 53727 | m-D9 | m-D9 | m-D9 | H | m-D9 | A-44 |
| 53728 | m-D9 | m-D9 | m-D9 | H | m-D9 | A-45 |
| 53729 | m-D10 | m-D10 | m-D10 | H | m-D10 | A-6 |
| 53730 | m-D10 | m-D10 | m-D10 | H | m-D10 | A-7 |
| 53731 | m-D10 | m-D10 | m-D10 | H | m-D10 | A-10 |
| 53732 | m-D10 | m-D10 | m-D10 | H | m-D10 | A-12 |
| 53733 | m-D10 | m-D10 | m-D10 | H | m-D10 | A-14 |
| 53734 | m-D10 | m-D10 | m-D10 | H | m-D10 | A-21 |
| 53735 | m-D10 | m-D10 | m-D10 | H | m-D10 | A-27 |
| 53736 | m-D10 | m-D10 | m-D10 | H | m-D10 | A-30 |
| 53737 | m-D10 | m-D10 | m-D10 | H | m-D10 | A-31 |
| 53738 | m-D10 | m-D10 | m-D10 | H | m-D10 | A-34 |
| 53739 | m-D10 | m-D10 | m-D10 | H | m-D10 | A-38 |
| 53740 | m-D10 | m-D10 | m-D10 | H | m-D10 | A-39 |
| 53741 | m-D10 | m-D10 | m-D10 | H | m-D10 | A-40 |
| 53742 | m-D10 | m-D10 | m-D10 | H | m-D10 | A-43 |
| 53743 | m-D10 | m-D10 | m-D10 | H | m-D10 | A-44 |
| 53744 | m-D10 | m-D10 | m-D10 | H | m-D10 | A-45 |
| 53745 | m-D11 | m-D11 | m-D11 | H | m-D11 | A-6 |
| 53746 | m-D11 | m-D11 | m-D11 | H | m-D11 | A-7 |
| 53747 | m-D11 | m-D11 | m-D11 | H | m-D11 | A-10 |
| 53748 | m-D11 | m-D11 | m-D11 | H | m-D11 | A-12 |
| 53749 | m-D11 | m-D11 | m-D11 | H | m-D11 | A-14 |
| 53750 | m-D11 | m-D11 | m-D11 | H | m-D11 | A-21 |
| 53751 | m-D11 | m-D11 | m-D11 | H | m-D11 | A-27 |
| 53752 | m-D11 | m-D11 | m-D11 | H | m-D11 | A-30 |
| 53753 | m-D11 | m-D11 | m-D11 | H | m-D11 | A-31 |
| 53754 | m-D11 | m-D11 | m-D11 | H | m-D11 | A-34 |
| 53755 | m-D11 | m-D11 | m-D11 | H | m-D11 | A-38 |
| 53756 | m-D11 | m-D11 | m-D11 | H | m-D11 | A-39 |
| 53757 | m-D11 | m-D11 | m-D11 | H | m-D11 | A-40 |
| 53758 | m-D11 | m-D11 | m-D11 | H | m-D11 | A-43 |
| 53759 | m-D11 | m-D11 | m-D11 | H | m-D11 | A-44 |
| 53760 | m-D11 | m-D11 | m-D11 | H | m-D11 | A-45 |
| 53761 | m-D12 | m-D12 | m-D12 | H | m-D12 | A-6 |
| 53762 | m-D12 | m-D12 | m-D12 | H | m-D12 | A-7 |
| 53763 | m-D12 | m-D12 | m-D12 | H | m-D12 | A-10 |
| 53764 | m-D12 | m-D12 | m-D12 | H | m-D12 | A-12 |
| 53765 | m-D12 | m-D12 | m-D12 | H | m-D12 | A-14 |
| 53766 | m-D12 | m-D12 | m-D12 | H | m-D12 | A-21 |
| 53767 | m-D12 | m-D12 | m-D12 | H | m-D12 | A-27 |
| 53768 | m-D12 | m-D12 | m-D12 | H | m-D12 | A-30 |
| 53769 | m-D12 | m-D12 | m-D12 | H | m-D12 | A-31 |
| 53770 | m-D12 | m-D12 | m-D12 | H | m-D12 | A-34 |
| 53771 | m-D12 | m-D12 | m-D12 | H | m-D12 | A-38 |
| 53772 | m-D12 | m-D12 | m-D12 | H | m-D12 | A-39 |
| 53773 | m-D12 | m-D12 | m-D12 | H | m-D12 | A-40 |
| 53774 | m-D12 | m-D12 | m-D12 | H | m-D12 | A-43 |
| 53775 | m-D12 | m-D12 | m-D12 | H | m-D12 | A-44 |
| 53776 | m-D12 | m-D12 | m-D12 | H | m-D12 | A-45 |
| 53777 | m-D13 | m-D13 | m-D13 | H | m-D13 | A-6 |
| 53778 | m-D13 | m-D13 | m-D13 | H | m-D13 | A-7 |
| 53779 | m-D13 | m-D13 | m-D13 | H | m-D13 | A-10 |
| 53780 | m-D13 | m-D13 | m-D13 | H | m-D13 | A-12 |
| 53781 | m-D13 | m-D13 | m-D13 | H | m-D13 | A-14 |
| 53782 | m-D13 | m-D13 | m-D13 | H | m-D13 | A-21 |
| 53783 | m-D13 | m-D13 | m-D13 | H | m-D13 | A-27 |
| 53784 | m-D13 | m-D13 | m-D13 | H | m-D13 | A-30 |
| 53785 | m-D13 | m-D13 | m-D13 | H | m-D13 | A-31 |
| 53786 | m-D13 | m-D13 | m-D13 | H | m-D13 | A-34 |
| 53787 | m-D13 | m-D13 | m-D13 | H | m-D13 | A-38 |
| 53788 | m-D13 | m-D13 | m-D13 | H | m-D13 | A-39 |
| 53789 | m-D13 | m-D13 | m-D13 | H | m-D13 | A-40 |
| 53790 | m-D13 | m-D13 | m-D13 | H | m-D13 | A-43 |
| 53791 | m-D13 | m-D13 | m-D13 | H | m-D13 | A-44 |
| 53792 | m-D13 | m-D13 | m-D13 | H | m-D13 | A-45 |
| 53793 | m-D14 | m-D14 | m-D14 | H | m-D14 | A-6 |
| 53794 | m-D14 | m-D14 | m-D14 | H | m-D14 | A-7 |
| 53795 | m-D14 | m-D14 | m-D14 | H | m-D14 | A-10 |
| 53796 | m-D14 | m-D14 | m-D14 | H | m-D14 | A-12 |
| 53797 | m-D14 | m-D14 | m-D14 | H | m-D14 | A-14 |
| 53798 | m-D14 | m-D14 | m-D14 | H | m-D14 | A-21 |
| 53799 | m-D14 | m-D14 | m-D14 | H | m-D14 | A-27 |
| 53800 | m-D14 | m-D14 | m-D14 | H | m-D14 | A-30 |
| 53801 | m-D14 | m-D14 | m-D14 | H | m-D14 | A-31 |
| 53802 | m-D14 | m-D14 | m-D14 | H | m-D14 | A-34 |
| 53803 | m-D14 | m-D14 | m-D14 | H | m-D14 | A-38 |
| 53804 | m-D14 | m-D14 | m-D14 | H | m-D14 | A-39 |
| 53805 | m-D14 | m-D14 | m-D14 | H | m-D14 | A-40 |
| 53806 | m-D14 | m-D14 | m-D14 | H | m-D14 | A-43 |
| 53807 | m-D14 | m-D14 | m-D14 | H | m-D14 | A-44 |
| 53808 | m-D14 | m-D14 | m-D14 | H | m-D14 | A-45 |
| 53767 | m-D12 | m-D12 | m-D12 | H | m-D12 | A-27 |
| 53768 | m-D12 | m-D12 | m-D12 | H | m-D12 | A-30 |
| 53769 | m-D12 | m-D12 | m-D12 | H | m-D12 | A-31 |
| 53770 | m-D12 | m-D12 | m-D12 | H | m-D12 | A-34 |
| 53771 | m-D12 | m-D12 | m-D12 | H | m-D12 | A-38 |
| 53772 | m-D12 | m-D12 | m-D12 | H | m-D12 | A-39 |
| 53773 | m-D12 | m-D12 | m-D12 | H | m-D12 | A-40 |
| 53774 | m-D12 | m-D12 | m-D12 | H | m-D12 | A-43 |
| 53775 | m-D12 | m-D12 | m-D12 | H | m-D12 | A-44 |
| 53776 | m-D12 | m-D12 | m-D12 | H | m-D12 | A-45 |
| 53777 | m-D13 | m-D13 | m-D13 | H | m-D13 | A-6 |
| 53778 | m-D13 | m-D13 | m-D13 | H | m-D13 | A-7 |
| 53779 | m-D13 | m-D13 | m-D13 | H | m-D13 | A-10 |
| 53780 | m-D13 | m-D13 | m-D13 | H | m-D13 | A-12 |
| 53781 | m-D13 | m-D13 | m-D13 | H | m-D13 | A-14 |
| 53782 | m-D13 | m-D13 | m-D13 | H | m-D13 | A-21 |
| 53783 | m-D13 | m-D13 | m-D13 | H | m-D13 | A-27 |
| 53784 | m-D13 | m-D13 | m-D13 | H | m-D13 | A-30 |
| 53785 | m-D13 | m-D13 | m-D13 | H | m-D13 | A-31 |
| 53786 | m-D13 | m-D13 | m-D13 | H | m-D13 | A-34 |
| 53787 | m-D13 | m-D13 | m-D13 | H | m-D13 | A-38 |
| 53788 | m-D13 | m-D13 | m-D13 | H | m-D13 | A-39 |
| 53789 | m-D13 | m-D13 | m-D13 | H | m-D13 | A-40 |
| 53790 | m-D13 | m-D13 | m-D13 | H | m-D13 | A-43 |
| 53791 | m-D13 | m-D13 | m-D13 | H | m-D13 | A-44 |
| 53792 | m-D13 | m-D13 | m-D13 | H | m-D13 | A-45 |
| 53793 | m-D14 | m-D14 | m-D14 | H | m-D14 | A-6 |
| 53794 | m-D14 | m-D14 | m-D14 | H | m-D14 | A-7 |
| 53795 | m-D14 | m-D14 | m-D14 | H | m-D14 | A-10 |
| 53796 | m-D14 | m-D14 | m-D14 | H | m-D14 | A-12 |
| 53797 | m-D14 | m-D14 | m-D14 | H | m-D14 | A-14 |
| 53798 | m-D14 | m-D14 | m-D14 | H | m-D14 | A-21 |
| 53799 | m-D14 | m-D14 | m-D14 | H | m-D14 | A-27 |
| 53800 | m-D14 | m-D14 | m-D14 | H | m-D14 | A-30 |
| 53801 | m-D14 | m-D14 | m-D14 | H | m-D14 | A-31 |
| 53802 | m-D14 | m-D14 | m-D14 | H | m-D14 | A-34 |
| 53803 | m-D14 | m-D14 | m-D14 | H | m-D14 | A-38 |
| 53804 | m-D14 | m-D14 | m-D14 | H | m-D14 | A-39 |
| 53805 | m-D14 | m-D14 | m-D14 | H | m-D14 | A-40 |
| 53806 | m-D14 | m-D14 | m-D14 | H | m-D14 | A-43 |
| 53807 | m-D14 | m-D14 | m-D14 | H | m-D14 | A-44 |
| 53808 | m-D14 | m-D14 | m-D14 | H | m-D14 | A-45 |
| 53869 | m-D4 | m-D4 | H | m-D4 | m-D4 | A-40 |
| 53870 | m-D4 | m-D4 | H | m-D4 | m-D4 | A-43 |
| 53871 | m-D4 | m-D4 | H | m-D4 | m-D4 | A-44 |
| 53872 | m-D4 | m-D4 | H | m-D4 | m-D4 | A-45 |
| 53873 | m-D5 | m-D5 | H | m-D5 | m-D5 | A-6 |
| 53874 | m-D5 | m-D5 | H | m-D5 | m-D5 | A-7 |
| 53875 | m-D5 | m-D5 | H | m-D5 | m-D5 | A-10 |
| 53876 | m-D5 | m-D5 | H | m-D5 | m-D5 | A-12 |
| 53877 | m-D5 | m-D5 | H | m-D5 | m-D5 | A-14 |
| 53878 | m-D5 | m-D5 | H | m-D5 | m-D5 | A-21 |
| 53879 | m-D5 | m-D5 | H | m-D5 | m-D5 | A-27 |
| 53880 | m-D5 | m-D5 | H | m-D5 | m-D5 | A-30 |
| 53881 | m-D5 | m-D5 | H | m-D5 | m-D5 | A-31 |
| 53882 | m-D5 | m-D5 | H | m-D5 | m-D5 | A-34 |
| 53883 | m-D5 | m-D5 | H | m-D5 | m-D5 | A-38 |
| 53884 | m-D5 | m-D5 | H | m-D5 | m-D5 | A-39 |
| 53885 | m-D5 | m-D5 | H | m-D5 | m-D5 | A-40 |
| 53886 | m-D5 | m-D5 | H | m-D5 | m-D5 | A-43 |
| 53887 | m-D5 | m-D5 | H | m-D5 | m-D5 | A-44 |
| 53888 | m-D5 | m-D5 | H | m-D5 | m-D5 | A-45 |
| 53889 | m-D6 | m-D6 | H | m-D6 | m-D6 | A-6 |
| 53890 | m-D6 | m-D6 | H | m-D6 | m-D6 | A-7 |
| 53891 | m-D6 | m-D6 | H | m-D6 | m-D6 | A-10 |
| 53892 | m-D6 | m-D6 | H | m-D6 | m-D6 | A-12 |
| 53893 | m-D6 | m-D6 | H | m-D6 | m-D6 | A-14 |
| 53894 | m-D6 | m-D6 | H | m-D6 | m-D6 | A-21 |
| 53895 | m-D6 | m-D6 | H | m-D6 | m-D6 | A-27 |
| 53896 | m-D6 | m-D6 | H | m-D6 | m-D6 | A-30 |
| 53897 | m-D6 | m-D6 | H | m-D6 | m-D6 | A-31 |
| 53898 | m-D6 | m-D6 | H | m-D6 | m-D6 | A-34 |
| 53899 | m-D6 | m-D6 | H | m-D6 | m-D6 | A-38 |
| 53900 | m-D6 | m-D6 | H | m-D6 | m-D6 | A-39 |
| 53901 | m-D6 | m-D6 | H | m-D6 | m-D6 | A-40 |
| 53902 | m-D6 | m-D6 | H | m-D6 | m-D6 | A-43 |
| 53903 | m-D6 | m-D6 | H | m-D6 | m-D6 | A-44 |
| 53904 | m-D6 | m-D6 | H | m-D6 | m-D6 | A-45 |
| 53905 | m-D7 | m-D7 | H | m-D7 | m-D7 | A-6 |
| 53906 | m-D7 | m-D7 | H | m-D7 | m-D7 | A-7 |
| 53907 | m-D7 | m-D7 | H | m-D7 | m-D7 | A-10 |
| 53908 | m-D7 | m-D7 | H | m-D7 | m-D7 | A-12 |
| 53909 | m-D7 | m-D7 | H | m-D7 | m-D7 | A-14 |
| 53910 | m-D7 | m-D7 | H | m-D7 | m-D7 | A-21 |
| 53911 | m-D7 | m-D7 | H | m-D7 | m-D7 | A-27 |
| 53912 | m-D7 | m-D7 | H | m-D7 | m-D7 | A-30 |
| 53913 | m-D7 | m-D7 | H | m-D7 | m-D7 | A-31 |
| 53914 | m-D7 | m-D7 | H | m-D7 | m-D7 | A-34 |
| 53915 | m-D7 | m-D7 | H | m-D7 | m-D7 | A-38 |
| 53916 | m-D7 | m-D7 | H | m-D7 | m-D7 | A-39 |
| 53917 | m-D7 | m-D7 | H | m-D7 | m-D7 | A-40 |
| 53918 | m-D7 | m-D7 | H | m-D7 | m-D7 | A-43 |
| 53919 | m-D7 | m-D7 | H | m-D7 | m-D7 | A-44 |
| 53920 | m-D7 | m-D7 | H | m-D7 | m-D7 | A-45 |
| 53921 | m-D8 | m-D8 | H | m-D8 | m-D8 | A-6 |
| 53922 | m-D8 | m-D8 | H | m-D8 | m-D8 | A-7 |
| 53923 | m-D8 | m-D8 | H | m-D8 | m-D8 | A-10 |
| 53924 | m-D8 | m-D8 | H | m-D8 | m-D8 | A-12 |
| 53925 | m-D8 | m-D8 | H | m-D8 | m-D8 | A-14 |
| 53926 | m-D8 | m-D8 | H | m-D8 | m-D8 | A-21 |
| 53927 | m-D8 | m-D8 | H | m-D8 | m-D8 | A-27 |
| 53928 | m-D8 | m-D8 | H | m-D8 | m-D8 | A-30 |
| 53929 | m-D8 | m-D8 | H | m-D8 | m-D8 | A-31 |
| 53930 | m-D8 | m-D8 | H | m-D8 | m-D8 | A-34 |
| 53931 | m-D8 | m-D8 | H | m-D8 | m-D8 | A-38 |
| 53932 | m-D8 | m-D8 | H | m-D8 | m-D8 | A-39 |
| 53933 | m-D8 | m-D8 | H | m-D8 | m-D8 | A-40 |
| 53934 | m-D8 | m-D8 | H | m-D8 | m-D8 | A-43 |
| 53935 | m-D8 | m-D8 | H | m-D8 | m-D8 | A-44 |
| 53936 | m-D8 | m-D8 | H | m-D8 | m-D8 | A-45 |
| 53937 | m-D9 | m-D9 | H | m-D9 | m-D9 | A-6 |
| 53938 | m-D9 | m-D9 | H | m-D9 | m-D9 | A-7 |
| 53939 | m-D9 | m-D9 | H | m-D9 | m-D9 | A-10 |
| 53940 | m-D9 | m-D9 | H | m-D9 | m-D9 | A-12 |
| 53941 | m-D9 | m-D9 | H | m-D9 | m-D9 | A-14 |
| 53942 | m-D9 | m-D9 | H | m-D9 | m-D9 | A-21 |
| 53943 | m-D9 | m-D9 | H | m-D9 | m-D9 | A-27 |
| 53944 | m-D9 | m-D9 | H | m-D9 | m-D9 | A-30 |
| 53945 | m-D9 | m-D9 | H | m-D9 | m-D9 | A-31 |
| 53946 | m-D9 | m-D9 | H | m-D9 | m-D9 | A-34 |
| 53947 | m-D9 | m-D9 | H | m-D9 | m-D9 | A-38 |
| 53948 | m-D9 | m-D9 | H | m-D9 | m-D9 | A-39 |
| 53949 | m-D9 | m-D9 | H | m-D9 | m-D9 | A-40 |
| 53950 | m-D9 | m-D9 | H | m-D9 | m-D9 | A-43 |
| 53951 | m-D9 | m-D9 | H | m-D9 | m-D9 | A-44 |
| 53952 | m-D9 | m-D9 | H | m-D9 | m-D9 | A-45 |
| 53953 | m-D10 | m-D10 | H | m-D10 | m-D10 | A-6 |
| 53954 | m-D10 | m-D10 | H | m-D10 | m-D10 | A-7 |
| 53955 | m-D10 | m-D10 | H | m-D10 | m-D10 | A-10 |
| 53956 | m-D10 | m-D10 | H | m-D10 | m-D10 | A-12 |
| 53957 | m-D10 | m-D10 | H | m-D10 | m-D10 | A-14 |
| 53958 | m-D10 | m-D10 | H | m-D10 | m-D10 | A-21 |
| 53959 | m-D10 | m-D10 | H | m-D10 | m-D10 | A-27 |
| 53960 | m-D10 | m-D10 | H | m-D10 | m-D10 | A-30 |
| 53961 | m-D10 | m-D10 | H | m-D10 | m-D10 | A-31 |
| 53962 | m-D10 | m-D10 | H | m-D10 | m-D10 | A-34 |
| 53963 | m-D10 | m-D10 | H | m-D10 | m-D10 | A-38 |
| 53964 | m-D10 | m-D10 | H | m-D10 | m-D10 | A-39 |
| 53965 | m-D10 | m-D10 | H | m-D10 | m-D10 | A-40 |
| 53966 | m-D10 | m-D10 | H | m-D10 | m-D10 | A-43 |
| 53967 | m-D10 | m-D10 | H | m-D10 | m-D10 | A-44 |
| 53968 | m-D10 | m-D10 | H | m-D10 | m-D10 | A-45 |
| 53969 | m-D11 | m-D11 | H | m-D11 | m-D11 | A-6 |
| 53970 | m-D11 | m-D11 | H | m-D11 | m-D11 | A-7 |
| 53971 | m-D11 | m-D11 | H | m-D11 | m-D11 | A-10 |
| 53972 | m-D11 | m-D11 | H | m-D11 | m-D11 | A-12 |
| 53973 | m-D11 | m-D11 | H | m-D11 | m-D11 | A-14 |
| 53974 | m-D11 | m-D11 | H | m-D11 | m-D11 | A-21 |
| 53975 | m-D11 | m-D11 | H | m-D11 | m-D11 | A-27 |
| 53976 | m-D11 | m-D11 | H | m-D11 | m-D11 | A-30 |
| 53977 | m-D11 | m-D11 | H | m-D11 | m-D11 | A-31 |
| 53978 | m-D11 | m-D11 | H | m-D11 | m-D11 | A-34 |
| 53979 | m-D11 | m-D11 | H | m-D11 | m-D11 | A-38 |
| 53980 | m-D11 | m-D11 | H | m-D11 | m-D11 | A-39 |
| 53981 | m-D11 | m-D11 | H | m-D11 | m-D11 | A-40 |
| 53982 | m-D11 | m-D11 | H | m-D11 | m-D11 | A-43 |
| 53983 | m-D11 | m-D11 | H | m-D11 | m-D11 | A-44 |
| 53984 | m-D11 | m-D11 | H | m-D11 | m-D11 | A-45 |
| 53985 | m-D12 | m-D12 | H | m-D12 | m-D12 | A-6 |
| 53986 | m-D12 | m-D12 | H | m-D12 | m-D12 | A-7 |
| 53987 | m-D12 | m-D12 | H | m-D12 | m-D12 | A-10 |
| 53988 | m-D12 | m-D12 | H | m-D12 | m-D12 | A-12 |
| 53989 | m-D12 | m-D12 | H | m-D12 | m-D12 | A-14 |
| 53990 | m-D12 | m-D12 | H | m-D12 | m-D12 | A-21 |
| 53991 | m-D12 | m-D12 | H | m-D12 | m-D12 | A-27 |
| 53992 | m-D12 | m-D12 | H | m-D12 | m-D12 | A-30 |
| 53993 | m-D12 | m-D12 | H | m-D12 | m-D12 | A-31 |
| 53994 | m-D12 | m-D12 | H | m-D12 | m-D12 | A-34 |
| 53995 | m-D12 | m-D12 | H | m-D12 | m-D12 | A-38 |
| 53996 | m-D12 | m-D12 | H | m-D12 | m-D12 | A-39 |
| 53997 | m-D12 | m-D12 | H | m-D12 | m-D12 | A-40 |
| 53998 | m-D12 | m-D12 | H | m-D12 | m-D12 | A-43 |
| 53999 | m-D12 | m-D12 | H | m-D12 | m-D12 | A-44 |
| 54000 | m-D12 | m-D12 | H | m-D12 | m-D12 | A-45 |
| 54001 | m-D13 | m-D13 | H | m-D13 | m-D13 | A-6 |
| 54002 | m-D13 | m-D13 | H | m-D13 | m-D13 | A-7 |
| 54003 | m-D13 | m-D13 | H | m-D13 | m-D13 | A-10 |
| 54004 | m-D13 | m-D13 | H | m-D13 | m-D13 | A-12 |
| 54005 | m-D13 | m-D13 | H | m-D13 | m-D13 | A-14 |
| 54006 | m-D13 | m-D13 | H | m-D13 | m-D13 | A-21 |
| 54007 | m-D13 | m-D13 | H | m-D13 | m-D13 | A-27 |
| 54008 | m-D13 | m-D13 | H | m-D13 | m-D13 | A-30 |
| 54009 | m-D13 | m-D13 | H | m-D13 | m-D13 | A-31 |
| 54010 | m-D13 | m-D13 | H | m-D13 | m-D13 | A-34 |
| 54011 | m-D13 | m-D13 | H | m-D13 | m-D13 | A-38 |
| 54012 | m-D13 | m-D13 | H | m-D13 | m-D13 | A-39 |
| 54013 | m-D13 | m-D13 | H | m-D13 | m-D13 | A-40 |
| 54014 | m-D13 | m-D13 | H | m-D13 | m-D13 | A-43 |
| 54015 | m-D13 | m-D13 | H | m-D13 | m-D13 | A-44 |
| 54016 | m-D13 | m-D13 | H | m-D13 | m-D13 | A-45 |
| 54017 | m-D14 | m-D14 | H | m-D14 | m-D14 | A-6 |
| 54018 | m-D14 | m-D14 | H | m-D14 | m-D14 | A-7 |
| 54019 | m-D14 | m-D14 | H | m-D14 | m-D14 | A-10 |
| 54020 | m-D14 | m-D14 | H | m-D14 | m-D14 | A-12 |
| 54021 | m-D14 | m-D14 | H | m-D14 | m-D14 | A-14 |
| 54022 | m-D14 | m-D14 | H | m-D14 | m-D14 | A-21 |
| 54023 | m-D14 | m-D14 | H | m-D14 | m-D14 | A-27 |
| 54024 | m-D14 | m-D14 | H | m-D14 | m-D14 | A-30 |
| 54025 | m-D14 | m-D14 | H | m-D14 | m-D14 | A-31 |
| 54026 | m-D14 | m-D14 | H | m-D14 | m-D14 | A-34 |
| 54027 | m-D14 | m-D14 | H | m-D14 | m-D14 | A-38 |
| 54028 | m-D14 | m-D14 | H | m-D14 | m-D14 | A-39 |
| 54029 | m-D14 | m-D14 | H | m-D14 | m-D14 | A-40 |
| 54030 | m-D14 | m-D14 | H | m-D14 | m-D14 | A-43 |
| 54031 | m-D14 | m-D14 | H | m-D14 | m-D14 | A-44 |
| 54032 | m-D14 | m-D14 | H | m-D14 | m-D14 | A-45 |
| 54033 | m-D1 | m-D1 | m-D1 | m-D1 | m-D1 | A-6 |
| 54034 | m-D1 | m-D1 | m-D1 | m-D1 | m-D1 | A-7 |
| 54035 | m-D1 | m-D1 | m-D1 | m-D1 | m-D1 | A-10 |
| 54036 | m-D1 | m-D1 | m-D1 | m-D1 | m-D1 | A-12 |
| 54037 | m-D1 | m-D1 | m-D1 | m-D1 | m-D1 | A-14 |
| 54038 | m-D1 | m-D1 | m-D1 | m-D1 | m-D1 | A-21 |
| 54039 | m-D1 | m-D1 | m-D1 | m-D1 | m-D1 | A-27 |
| 54040 | m-D1 | m-D1 | m-D1 | m-D1 | m-D1 | A-30 |
| 54041 | m-D1 | m-D1 | m-D1 | m-D1 | m-D1 | A-31 |
| 54042 | m-D1 | m-D1 | m-D1 | m-D1 | m-D1 | A-34 |
| 54043 | m-D1 | m-D1 | m-D1 | m-D1 | m-D1 | A-38 |
| 54044 | m-D1 | m-D1 | m-D1 | m-D1 | m-D1 | A-39 |
| 54045 | m-D1 | m-D1 | m-D1 | m-D1 | m-D1 | A-40 |
| 54046 | m-D1 | m-D1 | m-D1 | m-D1 | m-D1 | A-43 |
| 54047 | m-D1 | m-D1 | m-D1 | m-D1 | m-D1 | A-44 |
| 54048 | m-D1 | m-D1 | m-D1 | m-D1 | m-D1 | A-45 |
| 54049 | m-D2 | m-D2 | m-D2 | m-D2 | m-D2 | A-6 |
| 54050 | m-D2 | m-D2 | m-D2 | m-D2 | m-D2 | A-7 |
| 54051 | m-D2 | m-D2 | m-D2 | m-D2 | m-D2 | A-10 |
| 54052 | m-D2 | m-D2 | m-D2 | m-D2 | m-D2 | A-12 |
| 54053 | m-D2 | m-D2 | m-D2 | m-D2 | m-D2 | A-14 |
| 54054 | m-D2 | m-D2 | m-D2 | m-D2 | m-D2 | A-21 |
| 54055 | m-D2 | m-D2 | m-D2 | m-D2 | m-D2 | A-27 |
| 54056 | m-D2 | m-D2 | m-D2 | m-D2 | m-D2 | A-30 |
| 54057 | m-D2 | m-D2 | m-D2 | m-D2 | m-D2 | A-31 |
| 54058 | m-D2 | m-D2 | m-D2 | m-D2 | m-D2 | A-34 |
| 54059 | m-D2 | m-D2 | m-D2 | m-D2 | m-D2 | A-38 |
| 54060 | m-D2 | m-D2 | m-D2 | m-D2 | m-D2 | A-39 |
| 54061 | m-D2 | m-D2 | m-D2 | m-D2 | m-D2 | A-40 |
| 54062 | m-D2 | m-D2 | m-D2 | m-D2 | m-D2 | A-43 |
| 54063 | m-D2 | m-D2 | m-D2 | m-D2 | m-D2 | A-44 |
| 54064 | m-D2 | m-D2 | m-D2 | m-D2 | m-D2 | A-45 |
| 54065 | m-D3 | m-D3 | m-D3 | m-D3 | m-D3 | A-6 |
| 54066 | m-D3 | m-D3 | m-D3 | m-D3 | m-D3 | A-7 |
| 54067 | m-D3 | m-D3 | m-D3 | m-D3 | m-D3 | A-10 |
| 54068 | m-D3 | m-D3 | m-D3 | m-D3 | m-D3 | A-12 |
| 54069 | m-D3 | m-D3 | m-D3 | m-D3 | m-D3 | A-14 |
| 54070 | m-D3 | m-D3 | m-D3 | m-D3 | m-D3 | A-21 |
| 54071 | m-D3 | m-D3 | m-D3 | m-D3 | m-D3 | A-27 |
| 54072 | m-D3 | m-D3 | m-D3 | m-D3 | m-D3 | A-30 |
| 54073 | m-D3 | m-D3 | m-D3 | m-D3 | m-D3 | A-31 |
| 54074 | m-D3 | m-D3 | m-D3 | m-D3 | m-D3 | A-34 |
| 54075 | m-D3 | m-D3 | m-D3 | m-D3 | m-D3 | A-38 |
| 54076 | m-D3 | m-D3 | m-D3 | m-D3 | m-D3 | A-39 |
| 54077 | m-D3 | m-D3 | m-D3 | m-D3 | m-D3 | A-40 |
| 54078 | m-D3 | m-D3 | m-D3 | m-D3 | m-D3 | A-43 |
| 54079 | m-D3 | m-D3 | m-D3 | m-D3 | m-D3 | A-44 |
| 54080 | m-D3 | m-D3 | m-D3 | m-D3 | m-D3 | A-45 |
| 54081 | m-D4 | m-D4 | m-D4 | m-D4 | m-D4 | A-6 |
| 54082 | m-D4 | m-D4 | m-D4 | m-D4 | m-D4 | A-7 |
| 54083 | m-D4 | m-D4 | m-D4 | m-D4 | m-D4 | A-10 |
| 54084 | m-D4 | m-D4 | m-D4 | m-D4 | m-D4 | A-12 |
| 54085 | m-D4 | m-D4 | m-D4 | m-D4 | m-D4 | A-14 |
| 54086 | m-D4 | m-D4 | m-D4 | m-D4 | m-D4 | A-21 |
| 54087 | m-D4 | m-D4 | m-D4 | m-D4 | m-D4 | A-27 |
| 54088 | m-D4 | m-D4 | m-D4 | m-D4 | m-D4 | A-30 |
| 54089 | m-D4 | m-D4 | m-D4 | m-D4 | m-D4 | A-31 |
| 54090 | m-D4 | m-D4 | m-D4 | m-D4 | m-D4 | A-34 |
| 54091 | m-D4 | m-D4 | m-D4 | m-D4 | m-D4 | A-38 |
| 54092 | m-D4 | m-D4 | m-D4 | m-D4 | m-D4 | A-39 |
| 54093 | m-D4 | m-D4 | m-D4 | m-D4 | m-D4 | A-40 |
| 54094 | m-D4 | m-D4 | m-D4 | m-D4 | m-D4 | A-43 |
| 54095 | m-D4 | m-D4 | m-D4 | m-D4 | m-D4 | A-44 |
| 54096 | m-D4 | m-D4 | m-D4 | m-D4 | m-D4 | A-45 |
| 54097 | m-D5 | m-D5 | m-D5 | m-D5 | m-D5 | A-6 |
| 54098 | m-D5 | m-D5 | m-D5 | m-D5 | m-D5 | A-7 |
| 54099 | m-D5 | m-D5 | m-D5 | m-D5 | m-D5 | A-10 |
| 54100 | m-D5 | m-D5 | m-D5 | m-D5 | m-D5 | A-12 |
| 54101 | m-D5 | m-D5 | m-D5 | m-D5 | m-D5 | A-14 |
| 54102 | m-D5 | m-D5 | m-D5 | m-D5 | m-D5 | A-21 |
| 54103 | m-D5 | m-D5 | m-D5 | m-D5 | m-D5 | A-27 |
| 54104 | m-D5 | m-D5 | m-D5 | m-D5 | m-D5 | A-30 |
| 54105 | m-D5 | m-D5 | m-D5 | m-D5 | m-D5 | A-31 |
| 54106 | m-D5 | m-D5 | m-D5 | m-D5 | m-D5 | A-34 |
| 54107 | m-D5 | m-D5 | m-D5 | m-D5 | m-D5 | A-38 |
| 54108 | m-D5 | m-D5 | m-D5 | m-D5 | m-D5 | A-39 |
| 54109 | m-D5 | m-D5 | m-D5 | m-D5 | m-D5 | A-40 |
| 54110 | m-D5 | m-D5 | m-D5 | m-D5 | m-D5 | A-43 |
| 54111 | m-D5 | m-D5 | m-D5 | m-D5 | m-D5 | A-44 |
| 54112 | m-D5 | m-D5 | m-D5 | m-D5 | m-D5 | A-45 |
| 54113 | m-D6 | m-D6 | m-D6 | m-D6 | m-D6 | A-6 |
| 54114 | m-D6 | m-D6 | m-D6 | m-D6 | m-D6 | A-7 |
| 54115 | m-D6 | m-D6 | m-D6 | m-D6 | m-D6 | A-10 |
| 54116 | m-D6 | m-D6 | m-D6 | m-D6 | m-D6 | A-12 |
| 54117 | m-D6 | m-D6 | m-D6 | m-D6 | m-D6 | A-14 |
| 54118 | m-D6 | m-D6 | m-D6 | m-D6 | m-D6 | A-21 |
| 54119 | m-D6 | m-D6 | m-D6 | m-D6 | m-D6 | A-27 |
| 54120 | m-D6 | m-D6 | m-D6 | m-D6 | m-D6 | A-30 |
| 54121 | m-D6 | m-D6 | m-D6 | m-D6 | m-D6 | A-31 |
| 54122 | m-D6 | m-D6 | m-D6 | m-D6 | m-D6 | A-34 |
| 54123 | m-D6 | m-D6 | m-D6 | m-D6 | m-D6 | A-38 |
| 54124 | m-D6 | m-D6 | m-D6 | m-D6 | m-D6 | A-39 |
| 54125 | m-D6 | m-D6 | m-D6 | m-D6 | m-D6 | A-40 |
| 54126 | m-D6 | m-D6 | m-D6 | m-D6 | m-D6 | A-43 |
| 54127 | m-D6 | m-D6 | m-D6 | m-D6 | m-D6 | A-44 |
| 54128 | m-D6 | m-D6 | m-D6 | m-D6 | m-D6 | A-45 |
| 54129 | m-D7 | m-D7 | m-D7 | m-D7 | m-D7 | A-6 |
| 54130 | m-D7 | m-D7 | m-D7 | m-D7 | m-D7 | A-7 |
| 54131 | m-D7 | m-D7 | m-D7 | m-D7 | m-D7 | A-10 |
| 54132 | m-D7 | m-D7 | m-D7 | m-D7 | m-D7 | A-12 |
| 54133 | m-D7 | m-D7 | m-D7 | m-D7 | m-D7 | A-14 |
| 54134 | m-D7 | m-D7 | m-D7 | m-D7 | m-D7 | A-21 |
| 54135 | m-D7 | m-D7 | m-D7 | m-D7 | m-D7 | A-27 |
| 54136 | m-D7 | m-D7 | m-D7 | m-D7 | m-D7 | A-30 |
| 54137 | m-D7 | m-D7 | m-D7 | m-D7 | m-D7 | A-31 |
| 54138 | m-D7 | m-D7 | m-D7 | m-D7 | m-D7 | A-34 |
| 54139 | m-D7 | m-D7 | m-D7 | m-D7 | m-D7 | A-38 |
| 54140 | m-D7 | m-D7 | m-D7 | m-D7 | m-D7 | A-39 |
| 54141 | m-D7 | m-D7 | m-D7 | m-D7 | m-D7 | A-40 |
| 54142 | m-D7 | m-D7 | m-D7 | m-D7 | m-D7 | A-43 |
| 54143 | m-D7 | m-D7 | m-D7 | m-D7 | m-D7 | A-44 |
| 54144 | m-D7 | m-D7 | m-D7 | m-D7 | m-D7 | A-45 |
| 54145 | m-D8 | m-D8 | m-D8 | m-D8 | m-D8 | A-6 |
| 54146 | m-D8 | m-D8 | m-D8 | m-D8 | m-D8 | A-7 |
| 54147 | m-D8 | m-D8 | m-D8 | m-D8 | m-D8 | A-10 |
| 54148 | m-D8 | m-D8 | m-D8 | m-D8 | m-D8 | A-12 |
| 54149 | m-D8 | m-D8 | m-D8 | m-D8 | m-D8 | A-14 |
| 54150 | m-D8 | m-D8 | m-D8 | m-D8 | m-D8 | A-21 |
| 54151 | m-D8 | m-D8 | m-D8 | m-D8 | m-D8 | A-27 |
| 54152 | m-D8 | m-D8 | m-D8 | m-D8 | m-D8 | A-30 |
| 54153 | m-D8 | m-D8 | m-D8 | m-D8 | m-D8 | A-31 |
| 54154 | m-D8 | m-D8 | m-D8 | m-D8 | m-D8 | A-34 |
| 54155 | m-D8 | m-D8 | m-D8 | m-D8 | m-D8 | A-38 |
| 54156 | m-D8 | m-D8 | m-D8 | m-D8 | m-D8 | A-39 |
| 54157 | m-D8 | m-D8 | m-D8 | m-D8 | m-D8 | A-40 |
| 54158 | m-D8 | m-D8 | m-D8 | m-D8 | m-D8 | A-43 |
| 54159 | m-D8 | m-D8 | m-D8 | m-D8 | m-D8 | A-44 |
| 54160 | m-D8 | m-D8 | m-D8 | m-D8 | m-D8 | A-45 |
| 54161 | m-D9 | m-D9 | m-D9 | m-D9 | m-D9 | A-6 |
| 54162 | m-D9 | m-D9 | m-D9 | m-D9 | m-D9 | A-7 |
| 54163 | m-D9 | m-D9 | m-D9 | m-D9 | m-D9 | A-10 |
| 54164 | m-D9 | m-D9 | m-D9 | m-D9 | m-D9 | A-12 |
| 54165 | m-D9 | m-D9 | m-D9 | m-D9 | m-D9 | A-14 |
| 54166 | m-D9 | m-D9 | m-D9 | m-D9 | m-D9 | A-21 |
| 54167 | m-D9 | m-D9 | m-D9 | m-D9 | m-D9 | A-27 |
| 54168 | m-D9 | m-D9 | m-D9 | m-D9 | m-D9 | A-30 |
| 54169 | m-D9 | m-D9 | m-D9 | m-D9 | m-D9 | A-31 |
| 54170 | m-D9 | m-D9 | m-D9 | m-D9 | m-D9 | A-34 |
| 54171 | m-D9 | m-D9 | m-D9 | m-D9 | m-D9 | A-38 |
| 54172 | m-D9 | m-D9 | m-D9 | m-D9 | m-D9 | A-39 |
| 54173 | m-D9 | m-D9 | m-D9 | m-D9 | m-D9 | A-40 |
| 54174 | m-D9 | m-D9 | m-D9 | m-D9 | m-D9 | A-43 |
| 54175 | m-D9 | m-D9 | m-D9 | m-D9 | m-D9 | A-44 |
| 54176 | m-D9 | m-D9 | m-D9 | m-D9 | m-D9 | A-45 |
| 54177 | m-D10 | m-D10 | m-D10 | m-D10 | m-D10 | A-6 |
| 54178 | m-D10 | m-D10 | m-D10 | m-D10 | m-D10 | A-7 |
| 54179 | m-D10 | m-D10 | m-D10 | m-D10 | m-D10 | A-10 |
| 54180 | m-D10 | m-D10 | m-D10 | m-D10 | m-D10 | A-12 |
| 54181 | m-D10 | m-D10 | m-D10 | m-D10 | m-D10 | A-14 |
| 54182 | m-D10 | m-D10 | m-D10 | m-D10 | m-D10 | A-21 |
| 54183 | m-D10 | m-D10 | m-D10 | m-D10 | m-D10 | A-27 |
| 54184 | m-D10 | m-D10 | m-D10 | m-D10 | m-D10 | A-30 |
| 54185 | m-D10 | m-D10 | m-D10 | m-D10 | m-D10 | A-31 |
| 54186 | m-D10 | m-D10 | m-D10 | m-D10 | m-D10 | A-34 |
| 54187 | m-D10 | m-D10 | m-D10 | m-D10 | m-D10 | A-38 |
| 54188 | m-D10 | m-D10 | m-D10 | m-D10 | m-D10 | A-39 |
| 54189 | m-D10 | m-D10 | m-D10 | m-D10 | m-D10 | A-40 |
| 54190 | m-D10 | m-D10 | m-D10 | m-D10 | m-D10 | A-43 |
| 54191 | m-D10 | m-D10 | m-D10 | m-D10 | m-D10 | A-44 |
| 54192 | m-D10 | m-D10 | m-D10 | m-D10 | m-D10 | A-45 |
| 54193 | m-D11 | m-D11 | m-D11 | m-D11 | m-D11 | A-6 |
| 54194 | m-D11 | m-D11 | m-D11 | m-D11 | m-D11 | A-7 |
| 54195 | m-D11 | m-D11 | m-D11 | m-D11 | m-D11 | A-10 |
| 54196 | m-D11 | m-D11 | m-D11 | m-D11 | m-D11 | A-12 |
| 54197 | m-D11 | m-D11 | m-D11 | m-D11 | m-D11 | A-14 |
| 54198 | m-D11 | m-D11 | m-D11 | m-D11 | m-D11 | A-21 |
| 54199 | m-D11 | m-D11 | m-D11 | m-D11 | m-D11 | A-27 |
| 54200 | m-D11 | m-D11 | m-D11 | m-D11 | m-D11 | A-30 |
| 54201 | m-D11 | m-D11 | m-D11 | m-D11 | m-D11 | A-31 |
| 54202 | m-D11 | m-D11 | m-D11 | m-D11 | m-D11 | A-34 |
| 54203 | m-D11 | m-D11 | m-D11 | m-D11 | m-D11 | A-38 |
| 54204 | m-D11 | m-D11 | m-D11 | m-D11 | m-D11 | A-39 |
| 54205 | m-D11 | m-D11 | m-D11 | m-D11 | m-D11 | A-40 |
| 54206 | m-D11 | m-D11 | m-D11 | m-D11 | m-D11 | A-43 |
| 54207 | m-D11 | m-D11 | m-D11 | m-D11 | m-D11 | A-44 |
| 54208 | m-D11 | m-D11 | m-D11 | m-D11 | m-D11 | A-45 |
| 54209 | m-D12 | m-D12 | m-D12 | m-D12 | m-D12 | A-6 |
| 54210 | m-D12 | m-D12 | m-D12 | m-D12 | m-D12 | A-7 |
| 54211 | m-D12 | m-D12 | m-D12 | m-D12 | m-D12 | A-10 |
| 54212 | m-D12 | m-D12 | m-D12 | m-D12 | m-D12 | A-12 |
| 54213 | m-D12 | m-D12 | m-D12 | m-D12 | m-D12 | A-14 |
| 54214 | m-D12 | m-D12 | m-D12 | m-D12 | m-D12 | A-21 |
| 54215 | m-D12 | m-D12 | m-D12 | m-D12 | m-D12 | A-27 |
| 54216 | m-D12 | m-D12 | m-D12 | m-D12 | m-D12 | A-30 |
| 54217 | m-D12 | m-D12 | m-D12 | m-D12 | m-D12 | A-31 |
| 54218 | m-D12 | m-D12 | m-D12 | m-D12 | m-D12 | A-34 |
| 54219 | m-D12 | m-D12 | m-D12 | m-D12 | m-D12 | A-38 |
| 54220 | m-D12 | m-D12 | m-D12 | m-D12 | m-D12 | A-39 |
| 54221 | m-D12 | m-D12 | m-D12 | m-D12 | m-D12 | A-40 |
| 54222 | m-D12 | m-D12 | m-D12 | m-D12 | m-D12 | A-43 |
| 54223 | m-D12 | m-D12 | m-D12 | m-D12 | m-D12 | A-44 |
| 54224 | m-D12 | m-D12 | m-D12 | m-D12 | m-D12 | A-45 |
| 54225 | m-D13 | m-D13 | m-D13 | m-D13 | m-D13 | A-6 |
| 54226 | m-D13 | m-D13 | m-D13 | m-D13 | m-D13 | A-7 |
| 54227 | m-D13 | m-D13 | m-D13 | m-D13 | m-D13 | A-10 |
| 54228 | m-D13 | m-D13 | m-D13 | m-D13 | m-D13 | A-12 |
| 54229 | m-D13 | m-D13 | m-D13 | m-D13 | m-D13 | A-14 |
| 54230 | m-D13 | m-D13 | m-D13 | m-D13 | m-D13 | A-21 |
| 54231 | m-D13 | m-D13 | m-D13 | m-D13 | m-D13 | A-27 |
| 54232 | m-D13 | m-D13 | m-D13 | m-D13 | m-D13 | A-30 |
| 54233 | m-D13 | m-D13 | m-D13 | m-D13 | m-D13 | A-31 |
| 54234 | m-D13 | m-D13 | m-D13 | m-D13 | m-D13 | A-34 |
| 54235 | m-D13 | m-D13 | m-D13 | m-D13 | m-D13 | A-38 |
| 54236 | m-D13 | m-D13 | m-D13 | m-D13 | m-D13 | A-39 |
| 54237 | m-D13 | m-D13 | m-D13 | m-D13 | m-D13 | A-40 |
| 54238 | m-D13 | m-D13 | m-D13 | m-D13 | m-D13 | A-43 |
| 54239 | m-D13 | m-D13 | m-D13 | m-D13 | m-D13 | A-44 |
| 54240 | m-D13 | m-D13 | m-D13 | m-D13 | m-D13 | A-45 |
| 54241 | m-D14 | m-D14 | m-D14 | m-D14 | m-D14 | A-6 |
| 54242 | m-D14 | m-D14 | m-D14 | m-D14 | m-D14 | A-7 |
| 54243 | m-D14 | m-D14 | m-D14 | m-D14 | m-D14 | A-10 |
| 54244 | m-D14 | m-D14 | m-D14 | m-D14 | m-D14 | A-12 |
| 54245 | m-D14 | m-D14 | m-D14 | m-D14 | m-D14 | A-14 |
| 54246 | m-D14 | m-D14 | m-D14 | m-D14 | m-D14 | A-21 |
| 54247 | m-D14 | m-D14 | m-D14 | m-D14 | m-D14 | A-27 |
| 54248 | m-D14 | m-D14 | m-D14 | m-D14 | m-D14 | A-30 |
| 54249 | m-D14 | m-D14 | m-D14 | m-D14 | m-D14 | A-31 |
| 54250 | m-D14 | m-D14 | m-D14 | m-D14 | m-D14 | A-34 |
| 54251 | m-D14 | m-D14 | m-D14 | m-D14 | m-D14 | A-38 |
| 54252 | m-D14 | m-D14 | m-D14 | m-D14 | m-D14 | A-39 |
| 54253 | m-D14 | m-D14 | m-D14 | m-D14 | m-D14 | A-40 |
| 54254 | m-D14 | m-D14 | m-D14 | m-D14 | m-D14 | A-43 |
| 54255 | m-D14 | m-D14 | m-D14 | m-D14 | m-D14 | A-44 |
| 54256 | m-D14 | m-D14 | m-D14 | m-D14 | m-D14 | A-45 |

Specific examples of the compounds represented by the general formula (D) include compounds represented by the following general formula (D5) where Cz and A¹² each represent the group shown in the following Table 5.

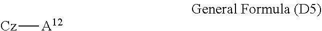

TABLE 15

| No. | Cz | A12 |
|---|---|---|
| 60001 | m-D1 | A-22 |
| 60002 | m-D1 | A-23 |
| 60003 | m-D1 | A-24 |
| 60004 | m-D1 | A-25 |
| 60005 | m-D1 | A-46 |
| 60006 | m-D1 | A-48 |
| 60007 | m-D2 | A-22 |
| 60008 | m-D2 | A-23 |
| 60009 | m-D2 | A-24 |
| 60010 | m-D2 | A-25 |
| 60011 | m-D2 | A-46 |
| 60012 | m-D2 | A-48 |
| 60013 | m-D3 | A-22 |
| 60014 | m-D3 | A-23 |
| 60015 | m-D3 | A-24 |
| 60016 | m-D3 | A-25 |
| 60017 | m-D3 | A-46 |
| 60018 | m-D3 | A-48 |
| 60019 | m-D4 | A-22 |
| 60020 | m-D4 | A-23 |
| 60021 | m-D4 | A-24 |
| 60022 | m-D4 | A-25 |
| 60023 | m-D4 | A-46 |
| 60024 | m-D4 | A-48 |
| 60025 | m-D5 | A-22 |
| 60026 | m-D5 | A-23 |
| 60027 | m-D5 | A-24 |
| 60028 | m-D5 | A-25 |
| 60029 | m-D5 | A-46 |
| 60030 | m-D5 | A-48 |
| 60031 | m-D6 | A-22 |
| 60032 | m-D6 | A-23 |
| 60033 | m-D6 | A-24 |
| 60034 | m-D6 | A-25 |
| 60035 | m-D6 | A-46 |
| 60036 | m-D6 | A-48 |
| 60037 | m-D7 | A-22 |
| 60038 | m-D7 | A-23 |
| 60039 | m-D7 | A-24 |
| 60040 | m-D7 | A-25 |
| 60041 | m-D7 | A-46 |
| 60042 | m-D7 | A-48 |
| 60043 | m-D8 | A-22 |
| 60044 | m-D8 | A-23 |
| 60045 | m-D8 | A-24 |
| 60046 | m-D8 | A-25 |
| 60047 | m-D8 | A-46 |
| 60048 | m-D8 | A-48 |
| 60049 | m-D9 | A-22 |
| 60050 | m-D9 | A-23 |
| 60051 | m-D9 | A-24 |
| 60052 | m-D9 | A-25 |
| 60053 | m-D9 | A-46 |
| 60054 | m-D9 | A-48 |
| 60055 | m-D10 | A-22 |
| 60056 | m-D10 | A-23 |
| 60057 | m-D10 | A-24 |
| 60058 | m-D10 | A-25 |
| 60059 | m-D10 | A-46 |
| 60060 | m-D10 | A-48 |
| 60061 | m-D11 | A-22 |
| 60062 | m-D11 | A-23 |
| 60063 | m-D11 | A-24 |
| 60064 | m-D11 | A-25 |
| 60065 | m-D11 | A-46 |
| 60066 | m-D11 | A-48 |
| 60067 | m-D12 | A-22 |
| 60068 | m-D12 | A-23 |
| 60069 | m-D12 | A-24 |
| 60070 | m-D12 | A-25 |
| 60071 | m-D12 | A-46 |
| 60072 | m-D12 | A-48 |
| 60073 | m-D13 | A-22 |
| 60074 | m-D13 | A-23 |
| 60075 | m-D13 | A-24 |
| 60076 | m-D13 | A-25 |
| 60077 | m-D13 | A-46 |
| 60078 | m-D13 | A-48 |
| 60079 | m-D14 | A-22 |
| 60080 | m-D14 | A-23 |
| 60081 | m-D14 | A-24 |
| 60082 | m-D14 | A-25 |
| 60083 | m-D14 | A-46 |
| 60084 | m-D14 | A-48 |

Next, compounds represented by the following general formula (E) are described below.

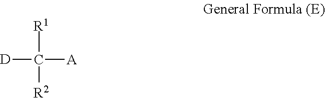

In the general formula (E), R¹ and R² each independently represent a fluoroalkyl group, D represents a substituent having a negative Hammett constant σₚ, and A represents a substituent having a positive Hammett constant UP.

As specific examples of the substituent that A includes, there are mentioned the specific examples (A-1 to A-78) of the substituent represented by A in the general formula (D).

In the following, specific examples of the compounds represented by the general formula (E) are shown.

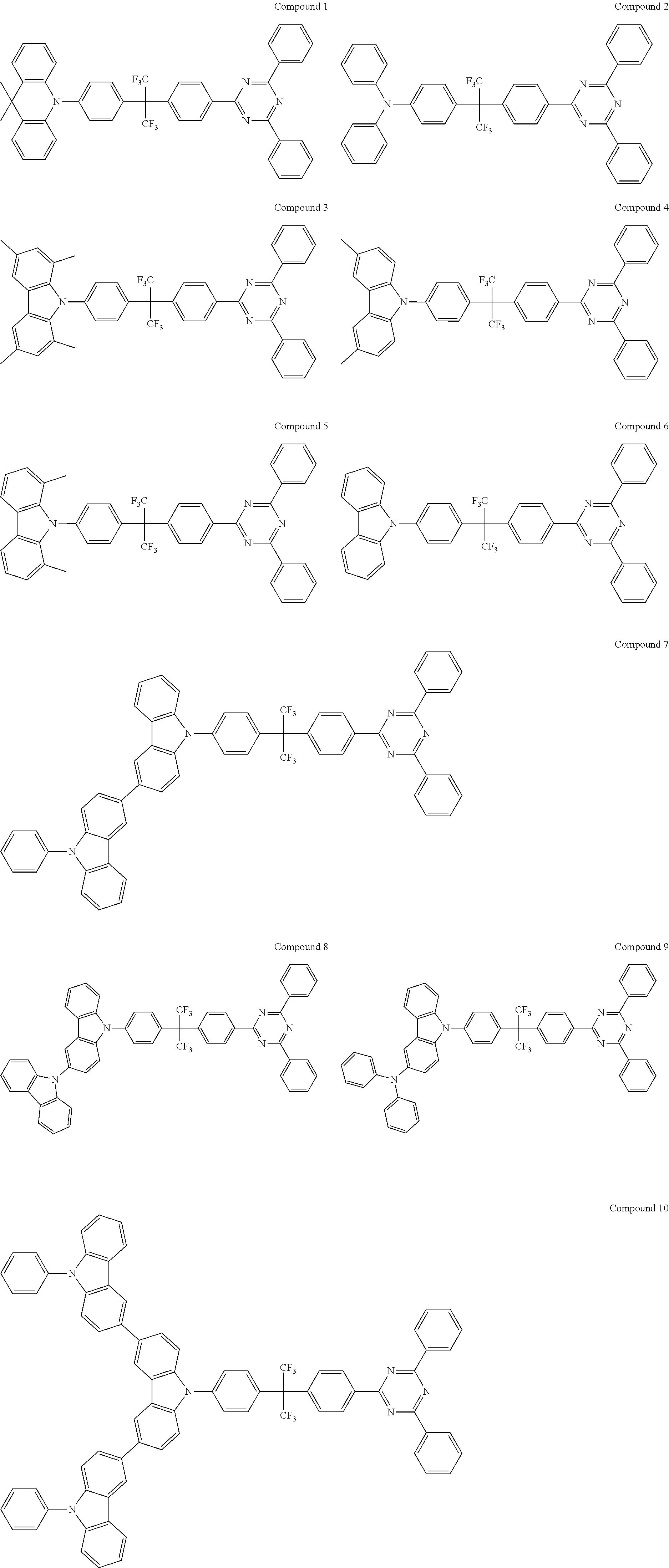

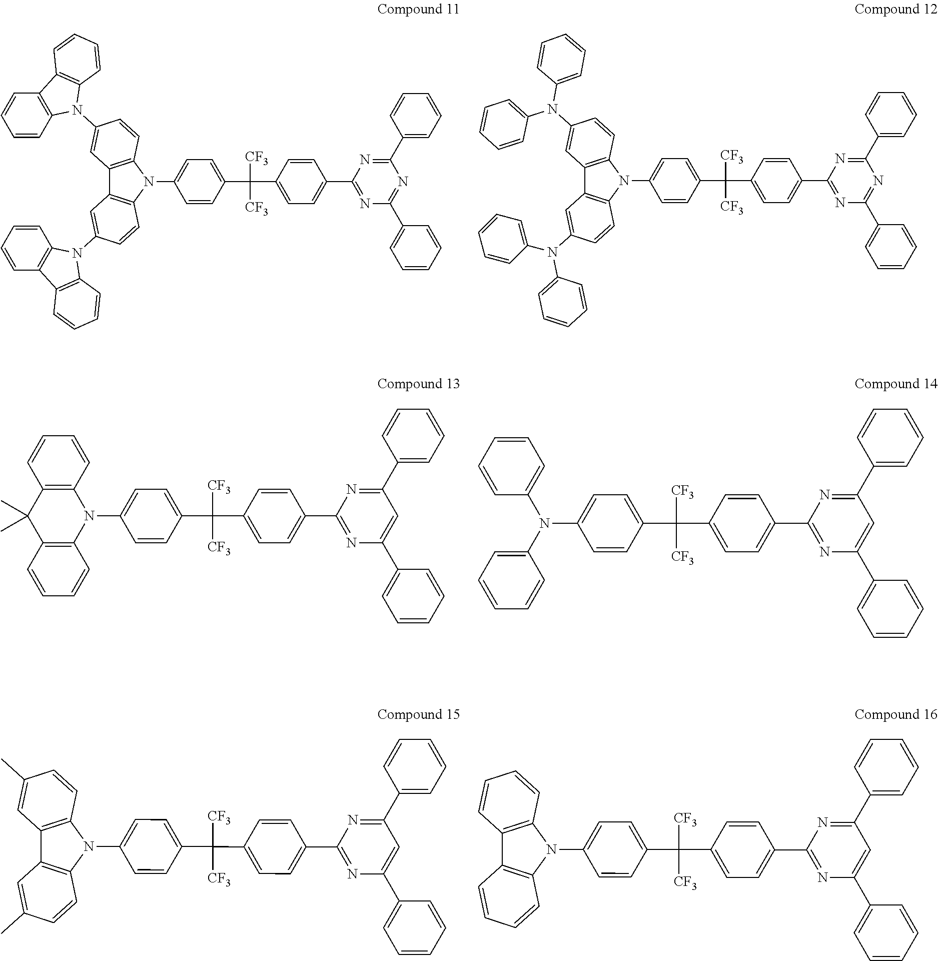

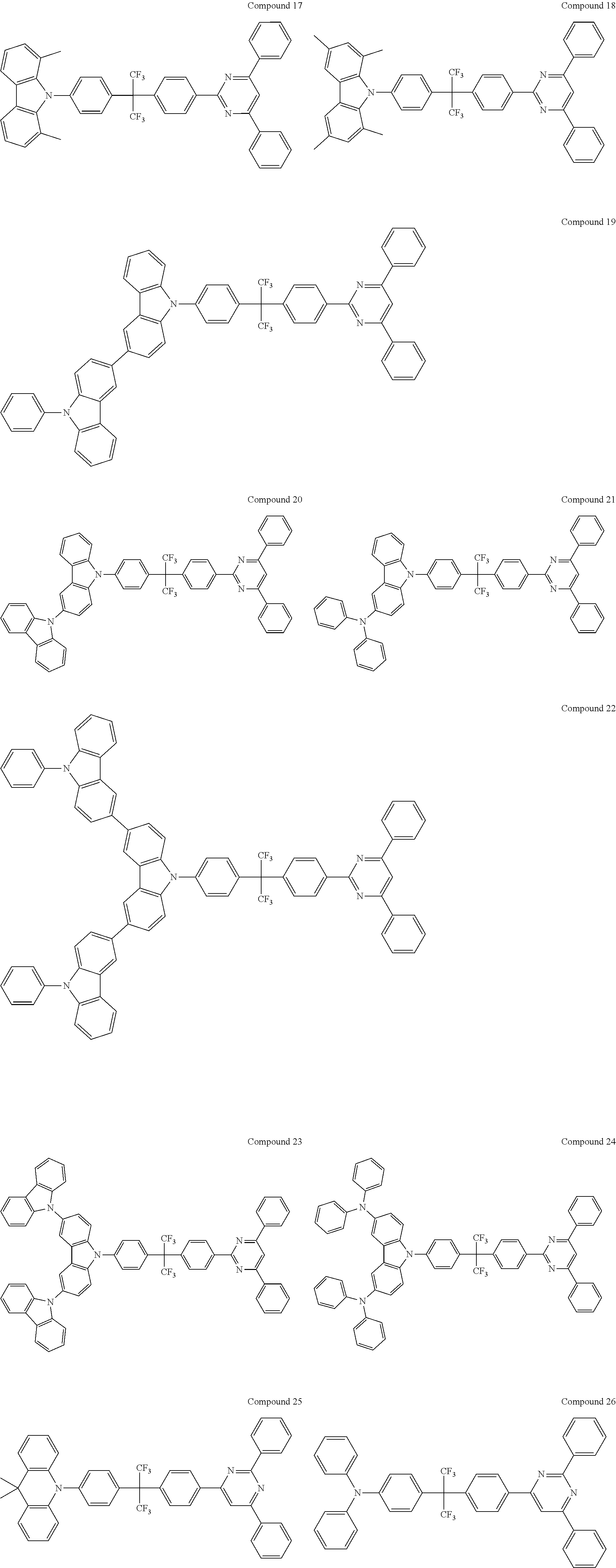

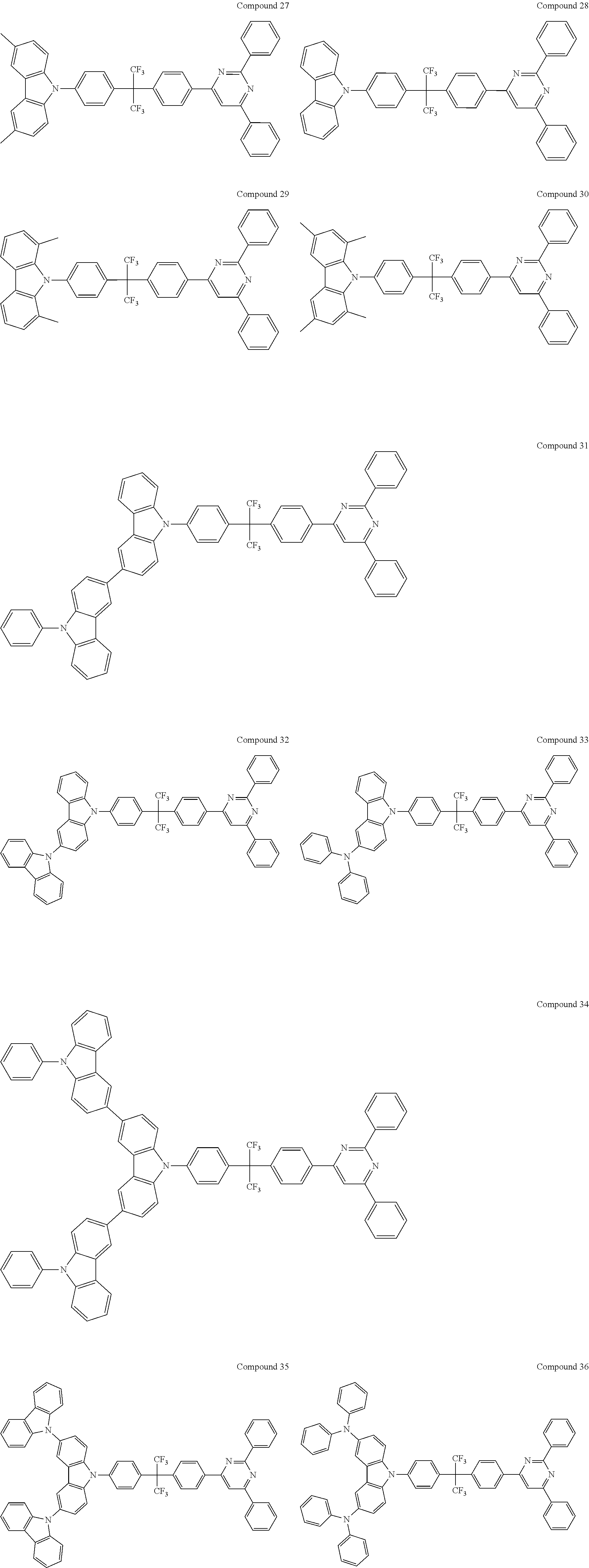

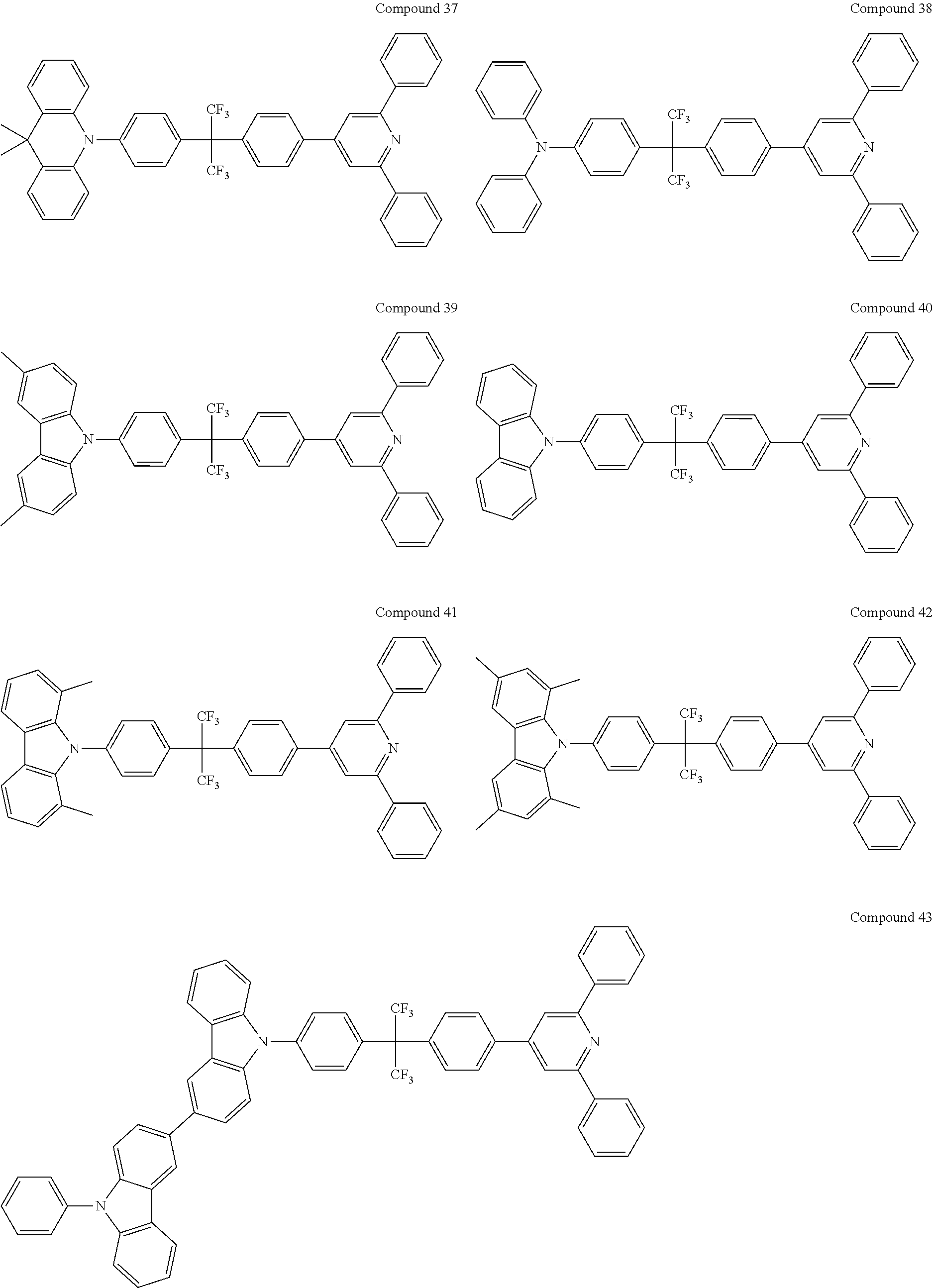

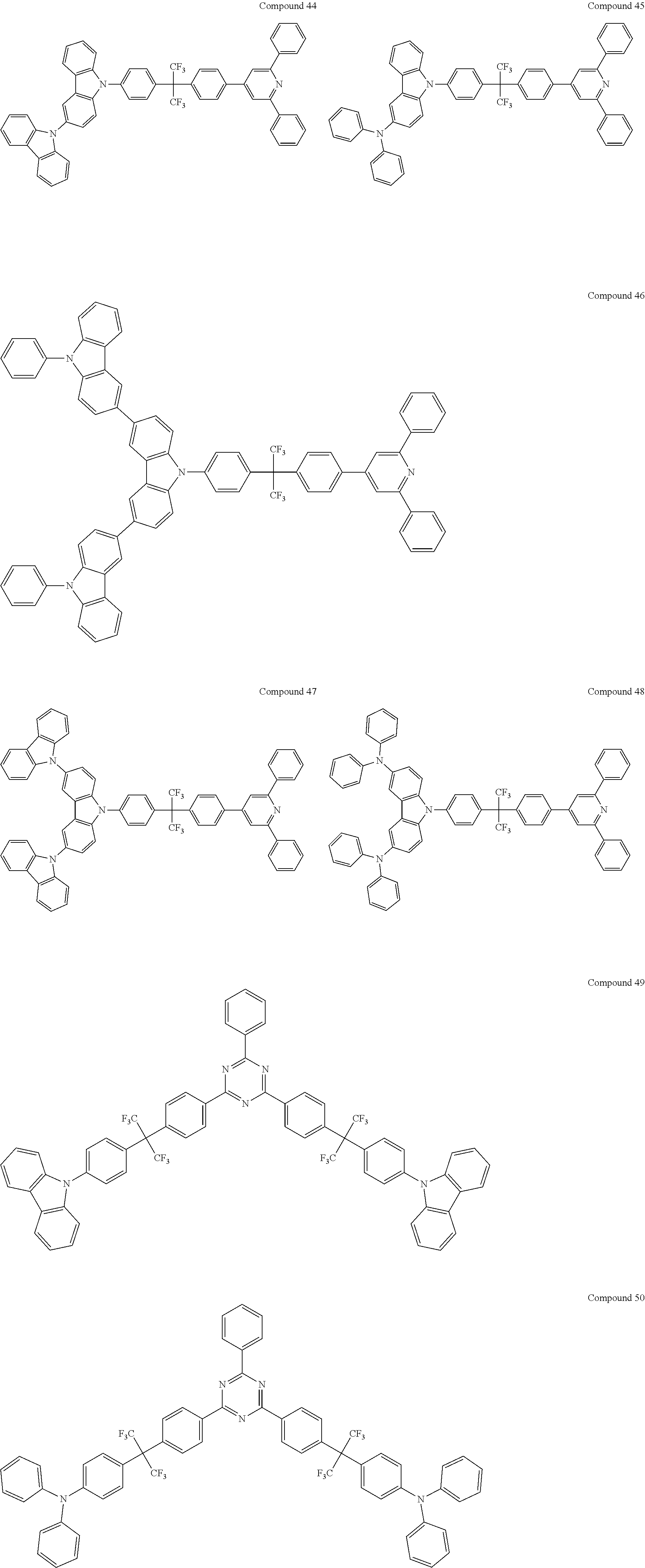

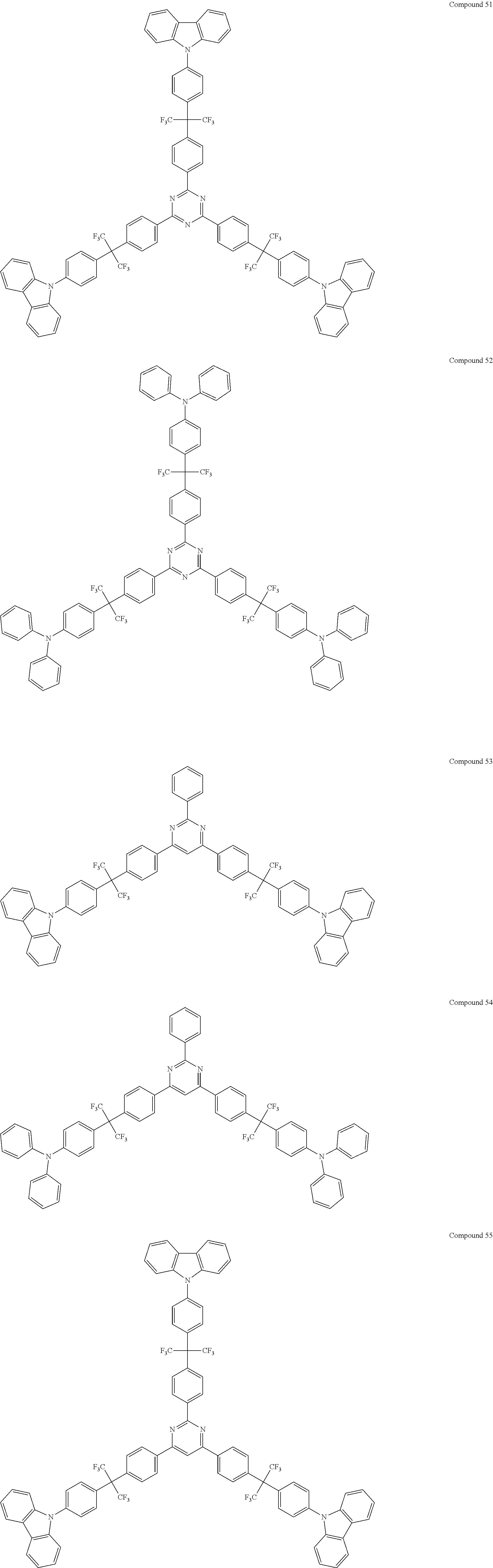

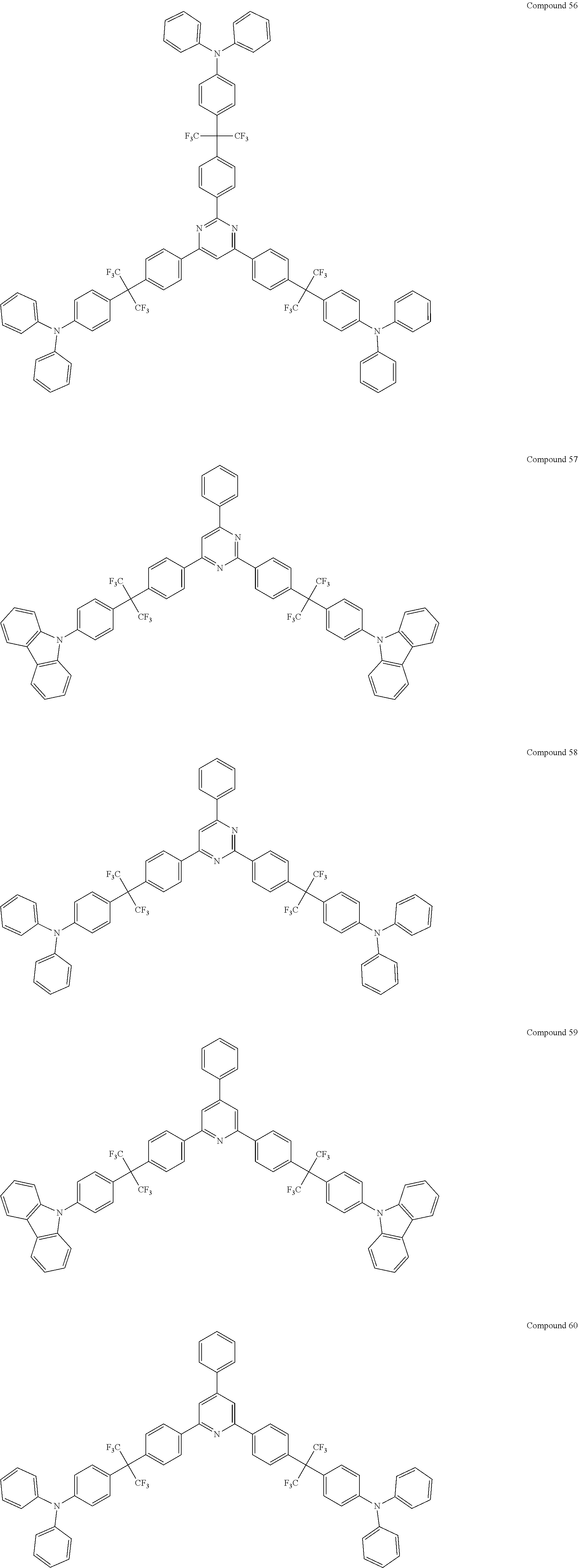

Next, compounds of the following general formula (F) are described.

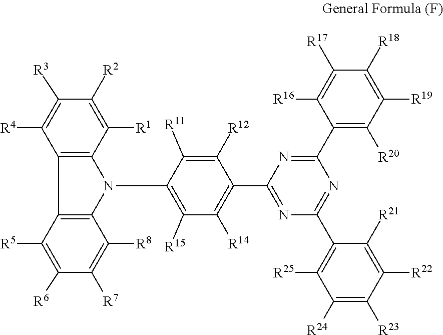

In the general formula (F), R¹ to R⁸, R¹², and R¹⁴ to R²⁵ each independently represent a hydrogen atom or a substituent, R¹¹ represents a substituted or unsubstituted alkyl group. However, at least one of R² to R⁴ is a substituted or unsubstituted alkyl group, and at least one of R⁵ to R⁷ is a substituted or unsubstituted alkyl group.

Specific examples of the compounds represented by the general formula (F) are shown below.

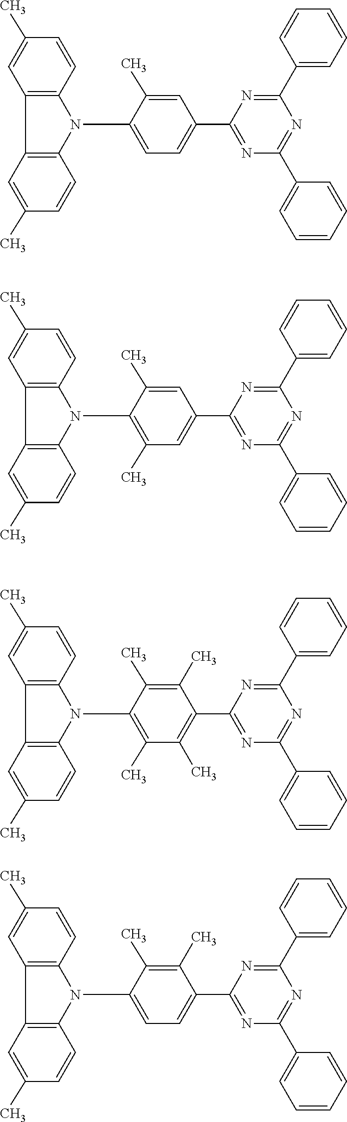

In addition to the light emitting materials represented by the above-mentioned general formulae, the following light emitting materials may also be employed.

### Light Emitting Material Group G

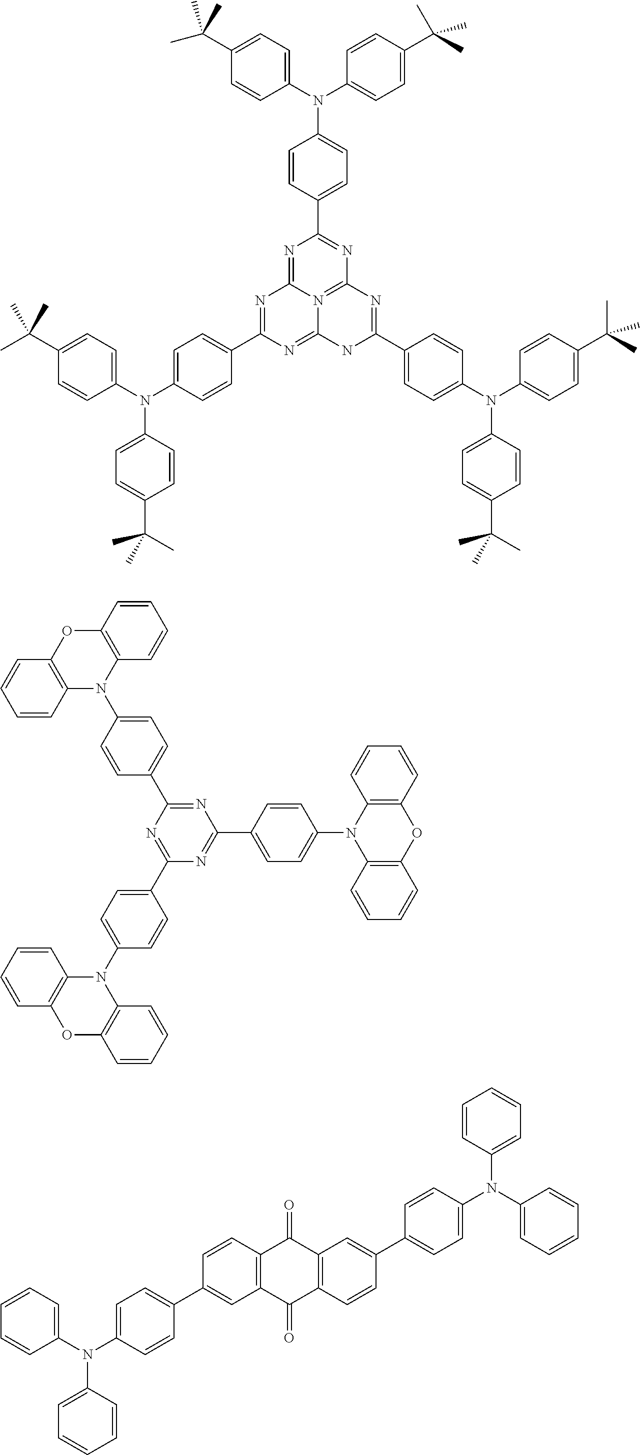

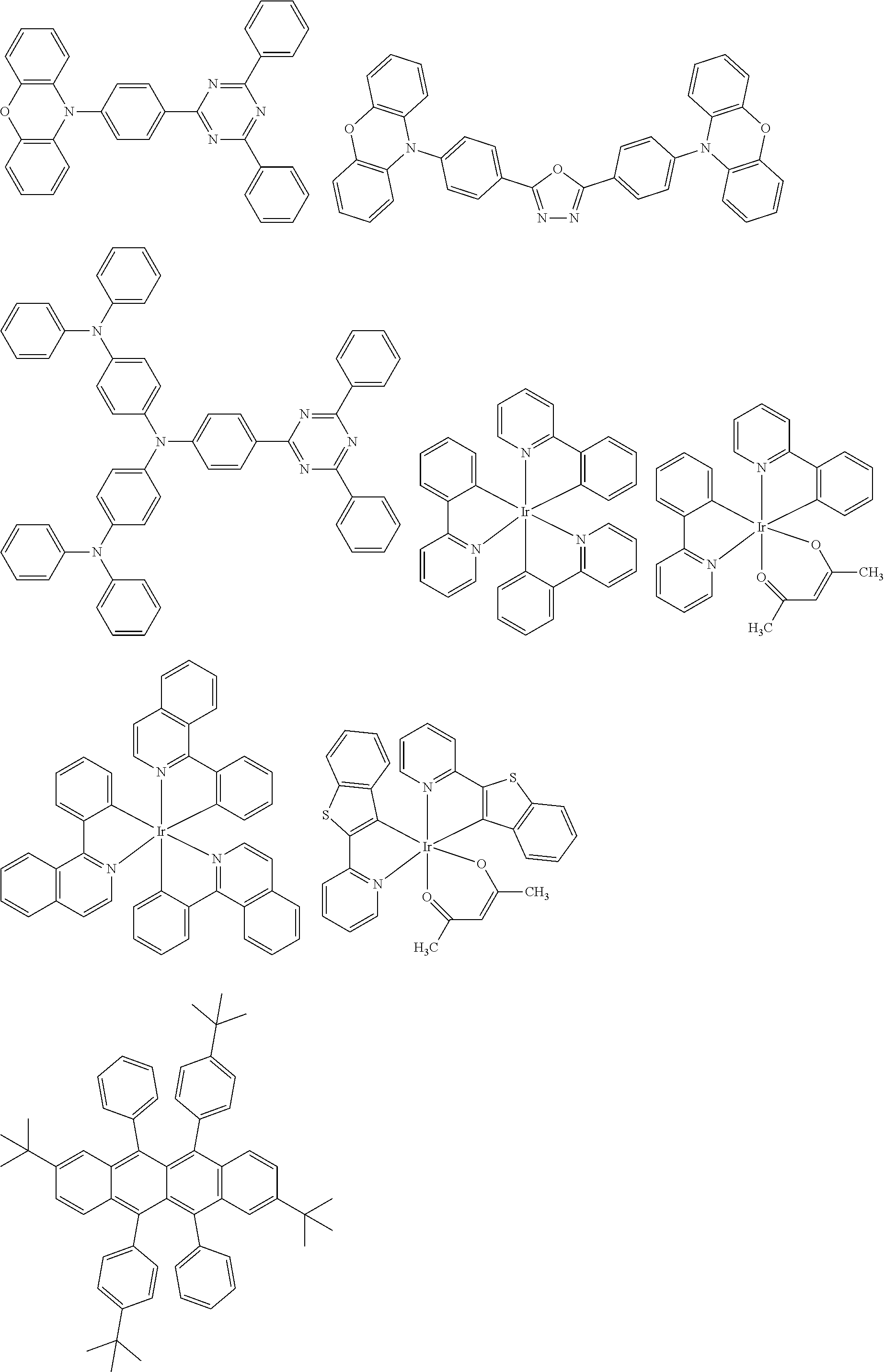

### (Injection Layer)

The injection layer is a layer that is provided between the electrode and the organic layer, for decreasing the driving voltage and enhancing the light emission luminance, and includes a hole injection layer and an electron injection layer, which may be provided between the anode and the light-emitting layer or the hole transport layer and between the cathode and the electron transport layer. The injection layer may be provided depending on necessity.

### (Blocking Layer)

The blocking layer is a layer that is capable of inhibiting charges (electrons or holes) and/or excitons present in the light-emitting layer from being diffused outside the light-emitting layer. The electron blocking layer may be disposed between the light-emitting layer and the hole transport layer, and inhibits electrons from passing through the light-emitting layer toward the hole transport layer. Similarly, the hole blocking layer may be disposed between the light-emitting layer and the electron transport layer, and inhibits holes from passing through the light-emitting layer toward the electron transport layer. The blocking layer may also be used for inhibiting excitons from being diffused outside the light-emitting layer. Thus, the electron blocking layer and the hole blocking layer each may also have a function as an exciton blocking layer. The term “the electron blocking layer” or “the exciton blocking layer” referred to herein is intended to include a layer that has both the functions of an electron blocking layer and an exciton blocking layer by one layer.

### (Hole Blocking Layer)

The hole blocking layer has the function of an electron transport layer in a broad sense. The hole blocking layer has a function of inhibiting holes from reaching the electron transport layer while transporting electrons, and thereby enhances the recombination probability of electrons and holes in the light-emitting layer. As the material for the hole blocking layer, the material for the electron transport layer to be mentioned below may be used optionally.

### (Electron Blocking Layer)

The electron blocking layer has the function of transporting holes in a broad sense. The electron blocking layer has a function of inhibiting electrons from reaching the hole transport layer while transporting holes, and thereby enhances the recombination probability of electrons and holes in the light-emitting layer.

### (Exciton Blocking Layer)

The exciton blocking layer is a layer for inhibiting excitons generated through recombination of holes and electrons in the light-emitting layer from being diffused to the charge transporting layer, and the use of the layer inserted enables effective confinement of excitons in the light-emitting layer, and thereby enhances the light emission efficiency of the device. The exciton blocking layer may be inserted adjacent to the light-emitting layer on any of the side of the anode and the side of the cathode, and on both the sides. Specifically, in the case where the exciton blocking layer is present on the side of the anode, the layer may be inserted between the hole transport layer and the light-emitting layer and adjacent to the light-emitting layer, and in the case where the layer is inserted on the side of the cathode, the layer may be inserted between the light-emitting layer and the cathode and adjacent to the light-emitting layer. Between the anode and the exciton blocking layer that is adjacent to the light-emitting layer on the side of the anode, a hole injection layer, an electron blocking layer and the like may be provided, and between the cathode and the exciton blocking layer that is adjacent to the light-emitting layer on the side of the cathode, an electron injection layer, an electron transport layer, a hole blocking layer and the like may be provided. In the case where the blocking layer is provided, preferably, at least one of the excited singlet energy and the higher than the excited singlet energy and the excited triplet energy of the light-emitting layer, respectively, of the light-emitting material.

(Hole transport Layer)

The hole transport layer is formed of a hole transport material having a function of transporting holes, and the hole transport layer may be provided as a single layer or plural layers.

The hole transport material has one of injection or transporting property of holes and blocking property of electrons, and may be any of an organic material and an inorganic material. Examples of known hole transport materials that may be used herein include a triazole derivative, an oxadiazole derivative, an imidazole derivative, a carbazole derivative, an indolocarbazole derivative, a polyarylalkane derivative, a pyrazoline derivative, a pyrazolone derivative, a phenylenediamine derivative, an arylamine derivative, an amino-substituted chalcone derivative, an oxazole derivative, a styrylanthracene derivative, a fluorenone derivative, a hydrazone derivative, a stilbene derivative, a silazane derivative, an aniline copolymer and an electroconductive polymer oligomer, particularly a thiophene oligomer. Among these, a porphyrin compound, an aromatic tertiary amine compound and a styrylamine compound are preferably used, and an aromatic tertiary amine compound is more preferably used.

(Electron transport Layer)

The electron transport layer is formed of a material having a function of transporting electrons, and the electron transport layer may be a single layer or may be formed of plural layers.

The electron transport material (often also acting as a hole blocking material) may have a function of transmitting the electrons injected from a cathode to a light-emitting layer. The electron transport layer usable here includes, for example, nitro-substituted fluorene derivatives, diphenylquinone derivatives, thiopyran dioxide derivatives, carbodiimides, fluorenylidenemethane derivatives, anthraquinodimethane and anthrone derivatives, oxadiazole derivatives, etc. Further, thiadiazole derivatives derived from the above-mentioned oxadiazole derivatives by substituting the oxygen atom in the oxadiazole ring with a sulfur atom, and quinoxaline derivatives having a quinoxaline ring known as an electron-attractive group are also usable as the electron transport material. Further, polymer materials prepared by introducing these materials into the polymer chain, or having these material in the polymer main chain are also usable.

In producing the organic electroluminescent device, the compound represented by the general formula (1) may be used not only in one organic layer (for example, light emitting layer) but also in plural organic layers. In so doing, the compound represented by the general formula (1) used in each organic layer may be the same as or different from each other. For example, the compound represented by the general formula (1) may be used in the above-mentioned injection layer, the blocking layer, the hole blocking layer, the electron blocking layer, the exciton blocking layer, the hole transport layer, and the electron transport layer in addition to the light emitting layer. The method for forming these layers is not specifically limited, and the layers may be formed according to any of a dry process or a wet process.

Preferred materials for use for the organic electroluminescent device are concretely exemplified below. However, the materials for use in the present invention are not limitatively interpreted by the following exemplary compounds. Compounds, even though exemplified as materials having a specific function, can also be used as other materials having any other function. R, R′, R₁ to R₁₀ in the structural formulae of the following exemplary compounds each independently represent a hydrogen atom or a substituent. X represent a carbon atom or a hetero atom to form the ring skeleton, n represents an integer of 3 to 5, Y represents a substituent, and m represents an integer of 0 or more.

As a host material in the light emitting layer, use of the compound represented by the general formula (1) is most preferred, but in the case where the compound represented by the general formula (1) is used as any other than a host material (for example, as a hole blocking material or an electron transport material), any other compound than those represented by the general formula (1) may be used as a host material. Examples of compounds usable as a host material are mentioned below.

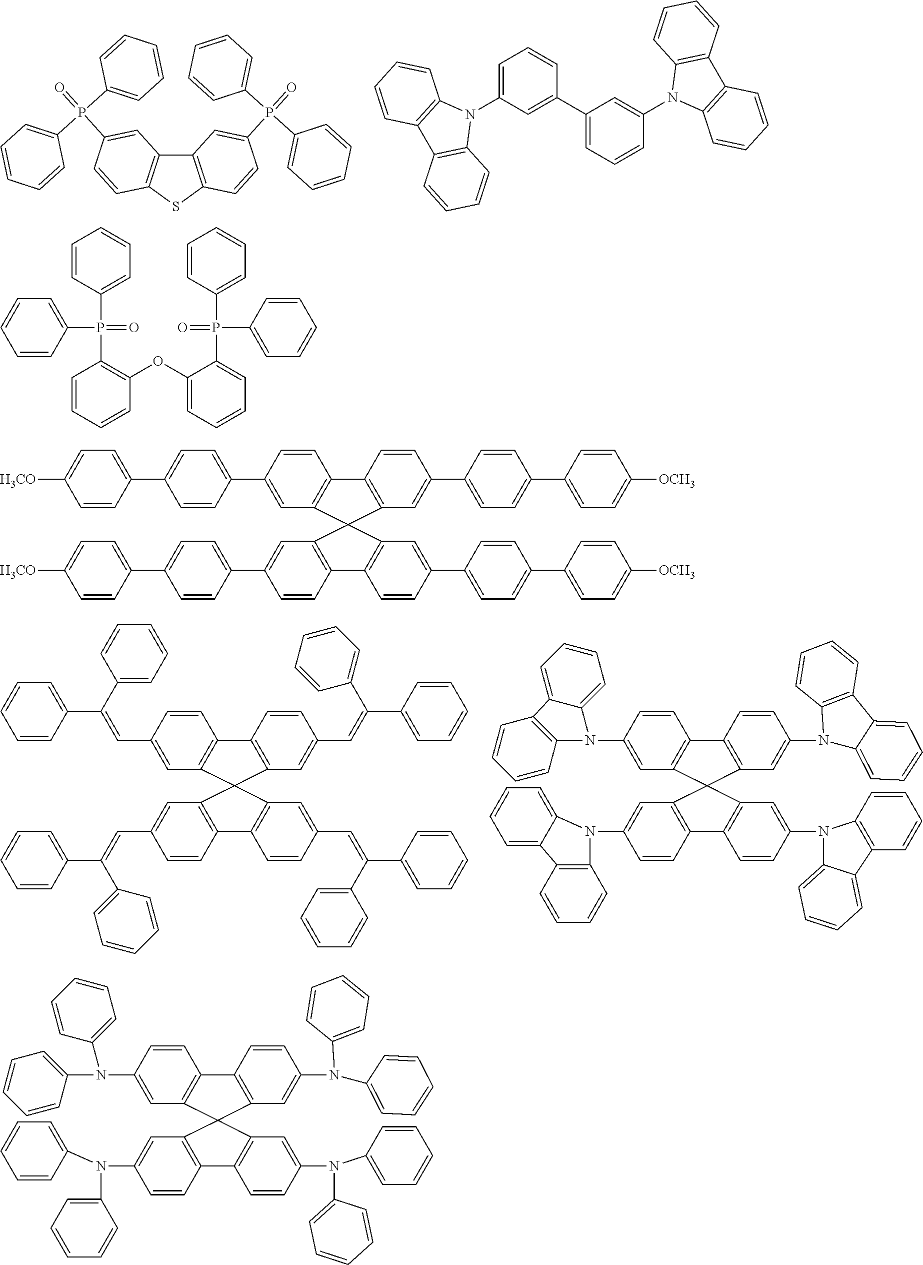

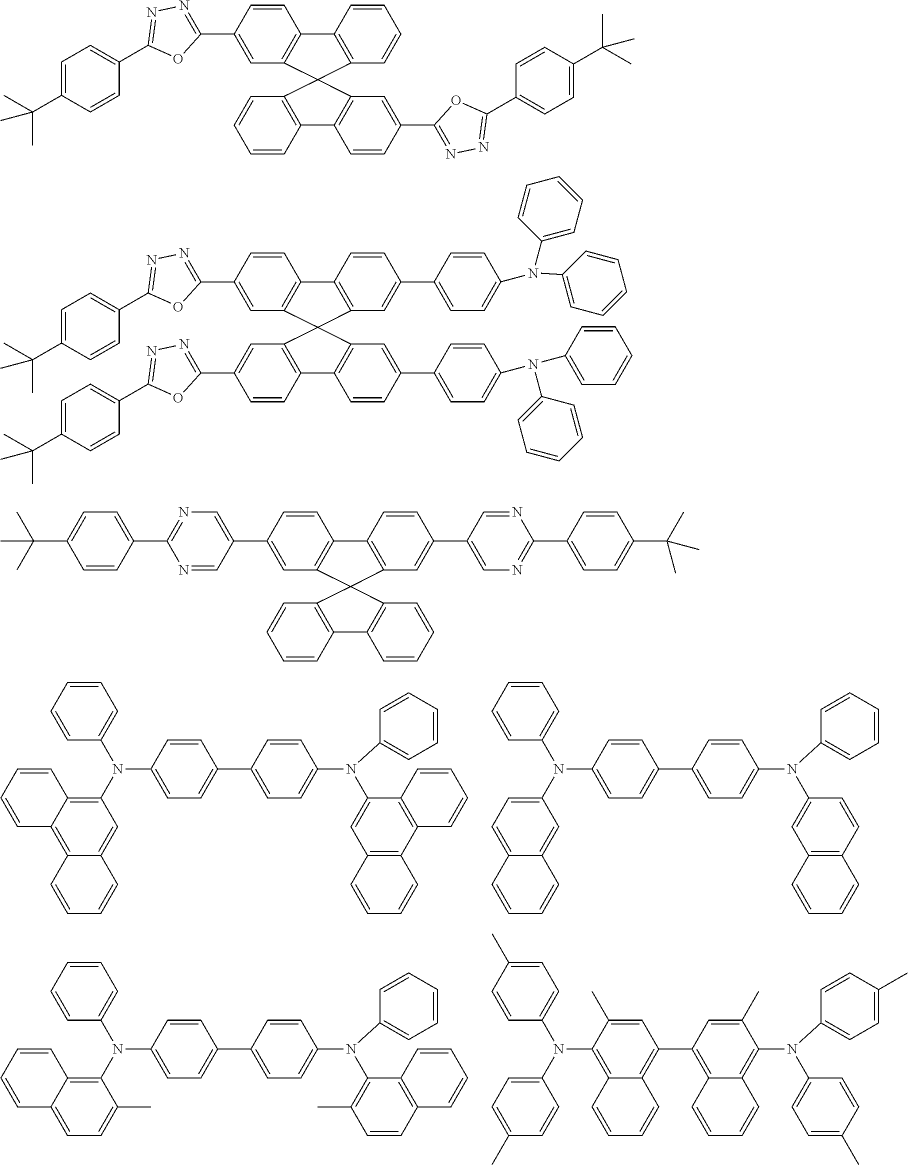

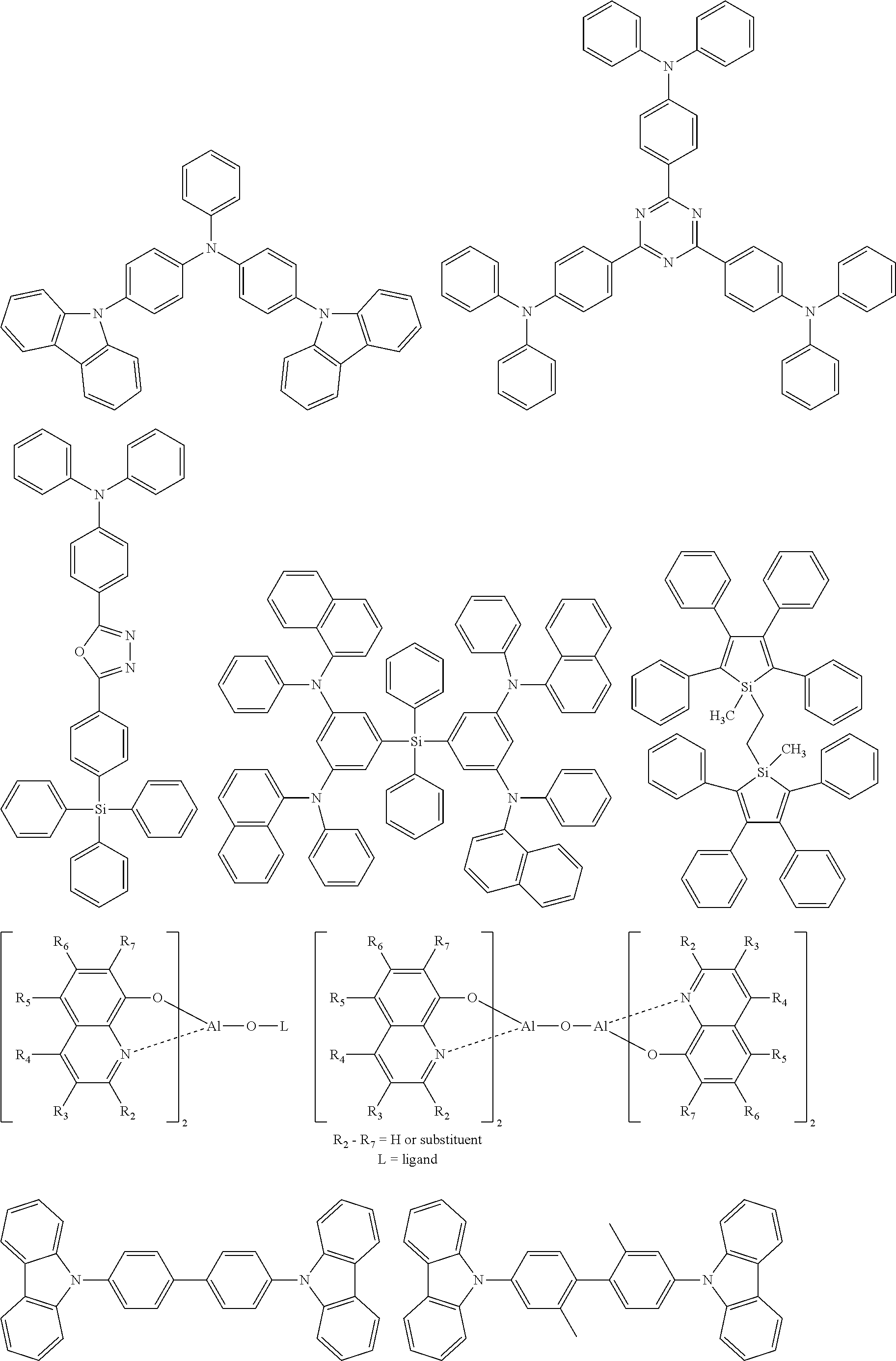

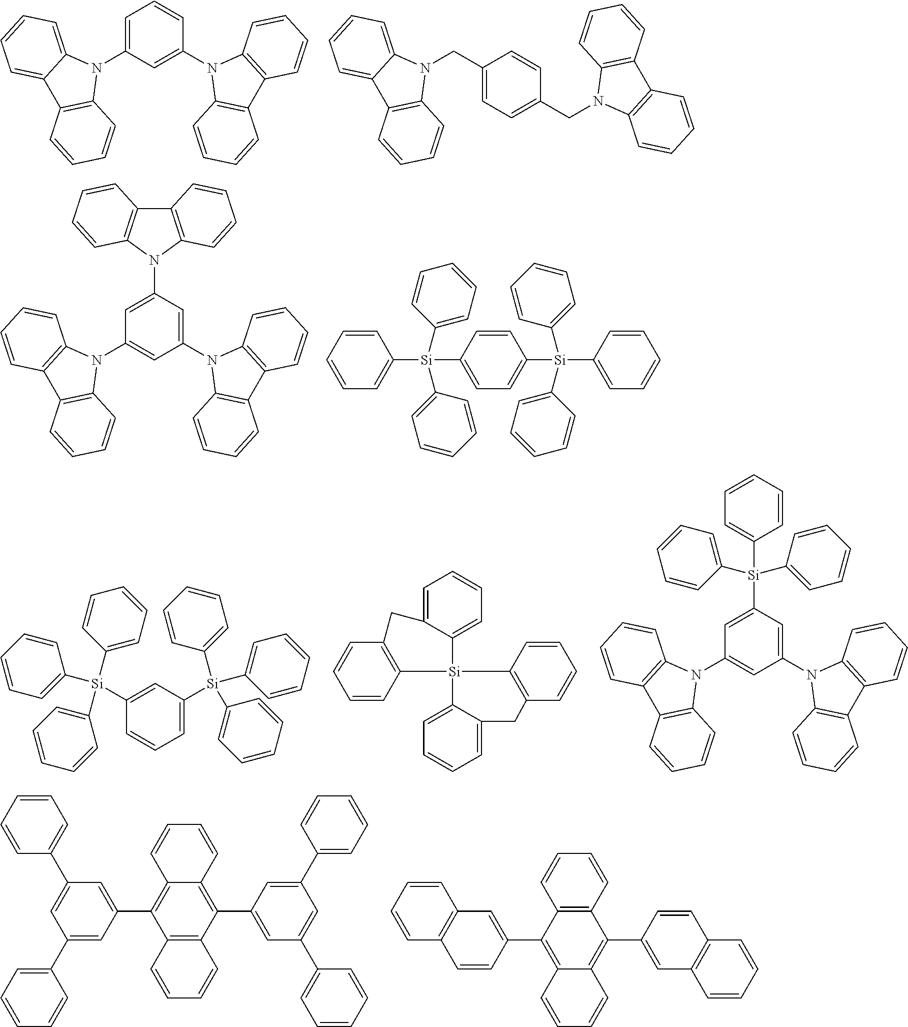

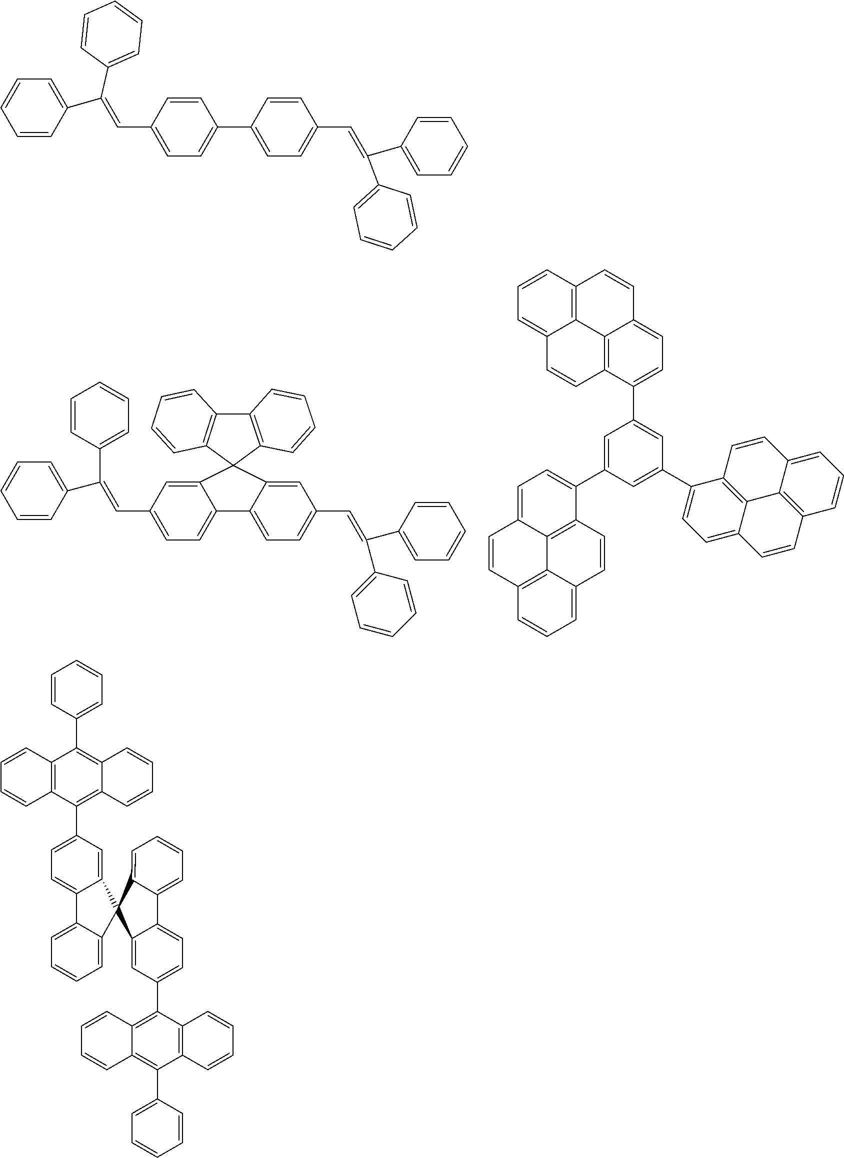

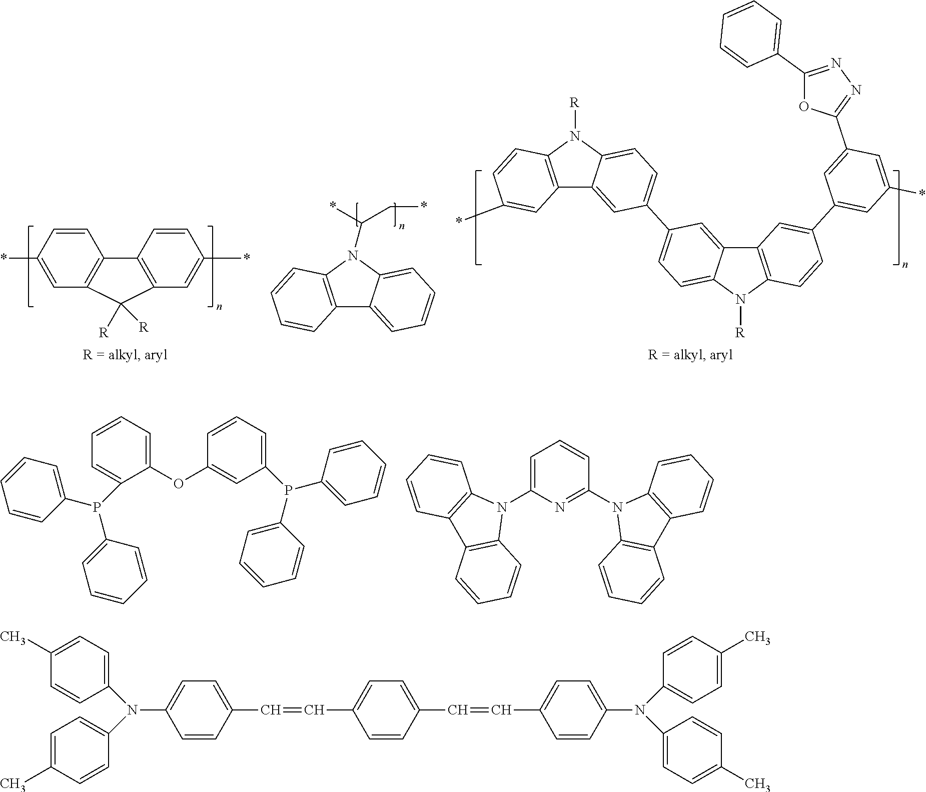

Next, preferred compounds for use as a hole injection material are mentioned below.

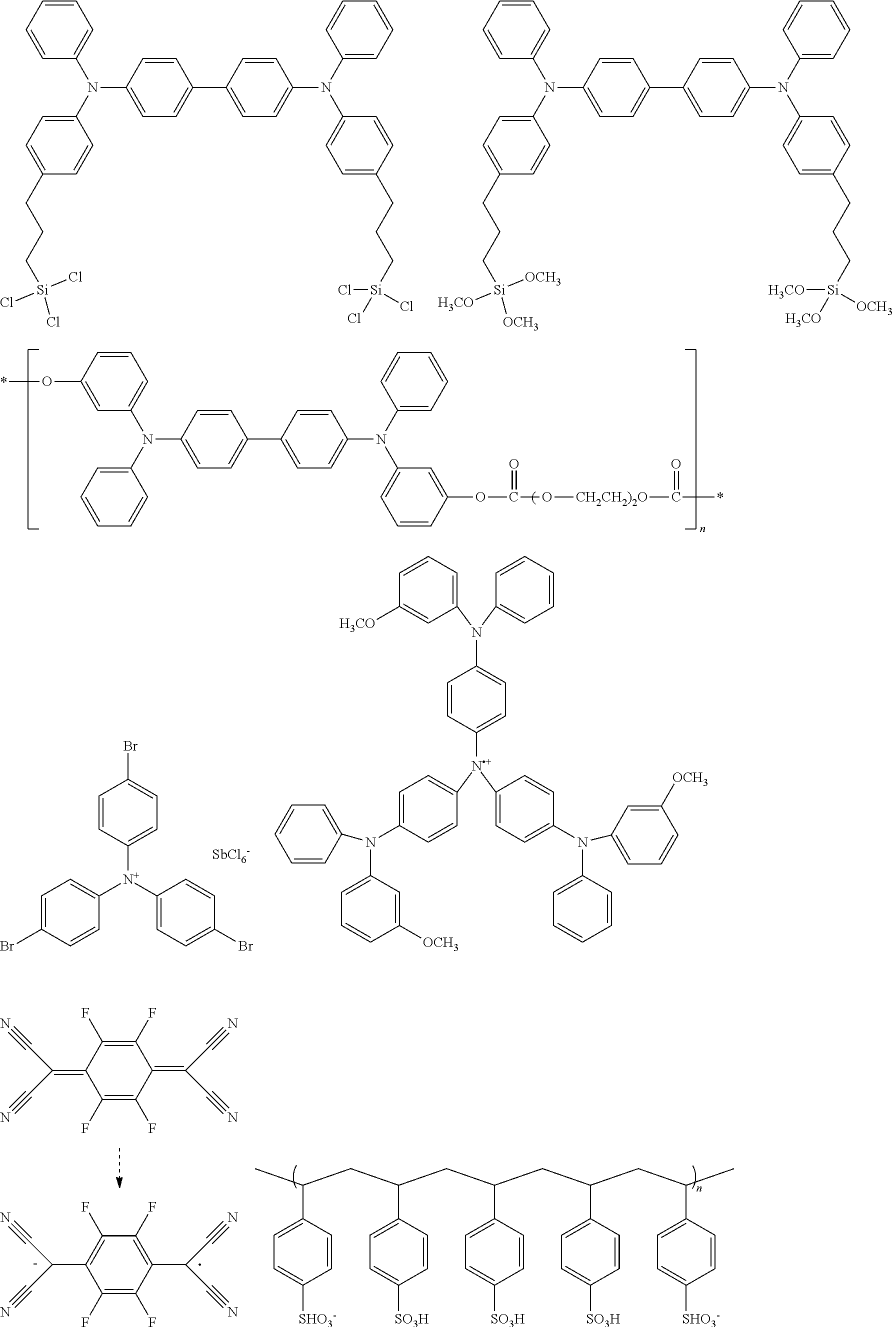

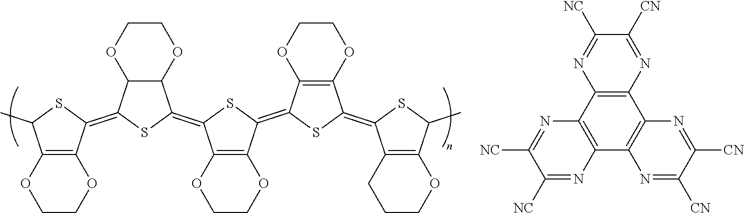

Next, preferred compounds for use as a hole transport material are mentioned below.

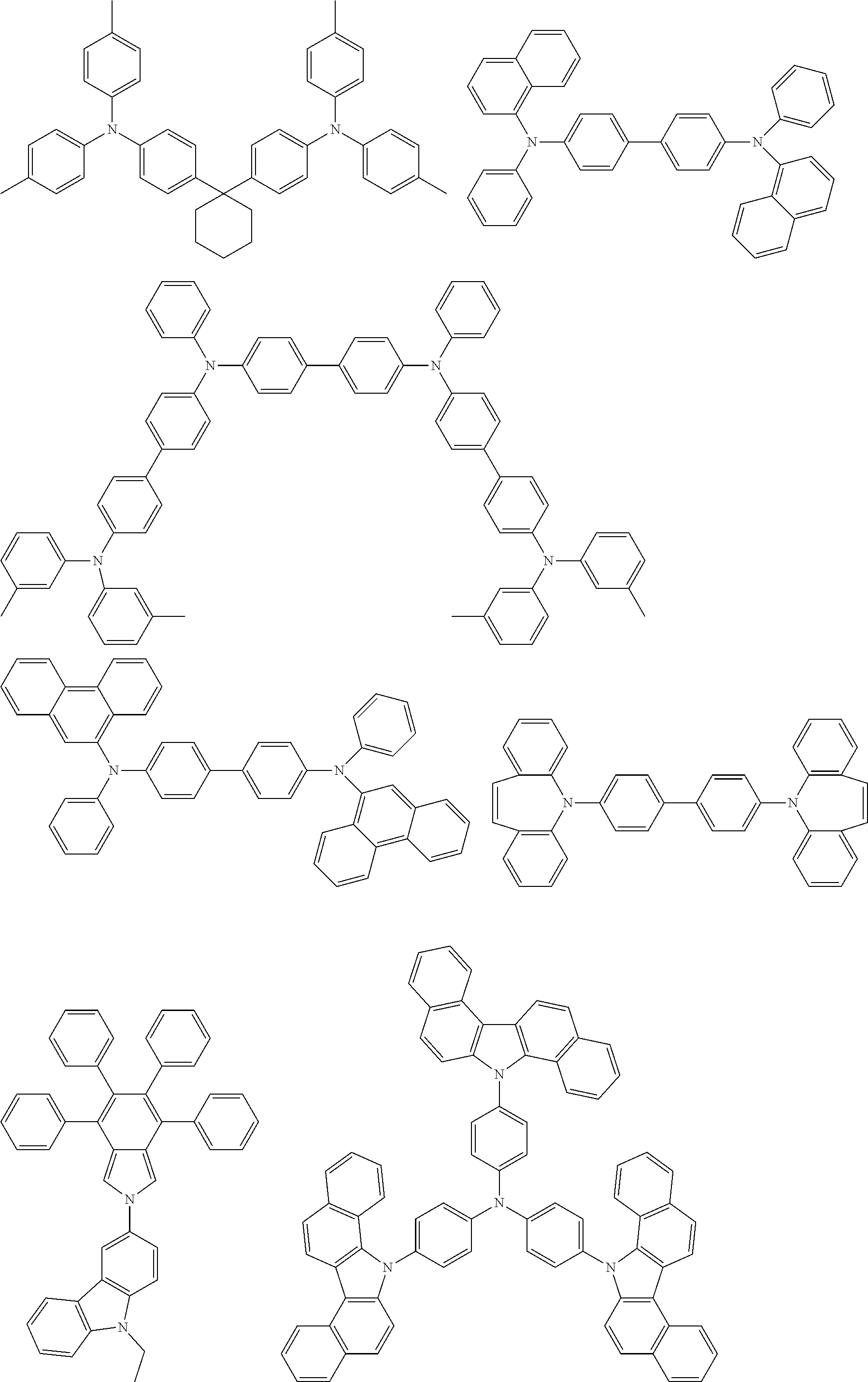

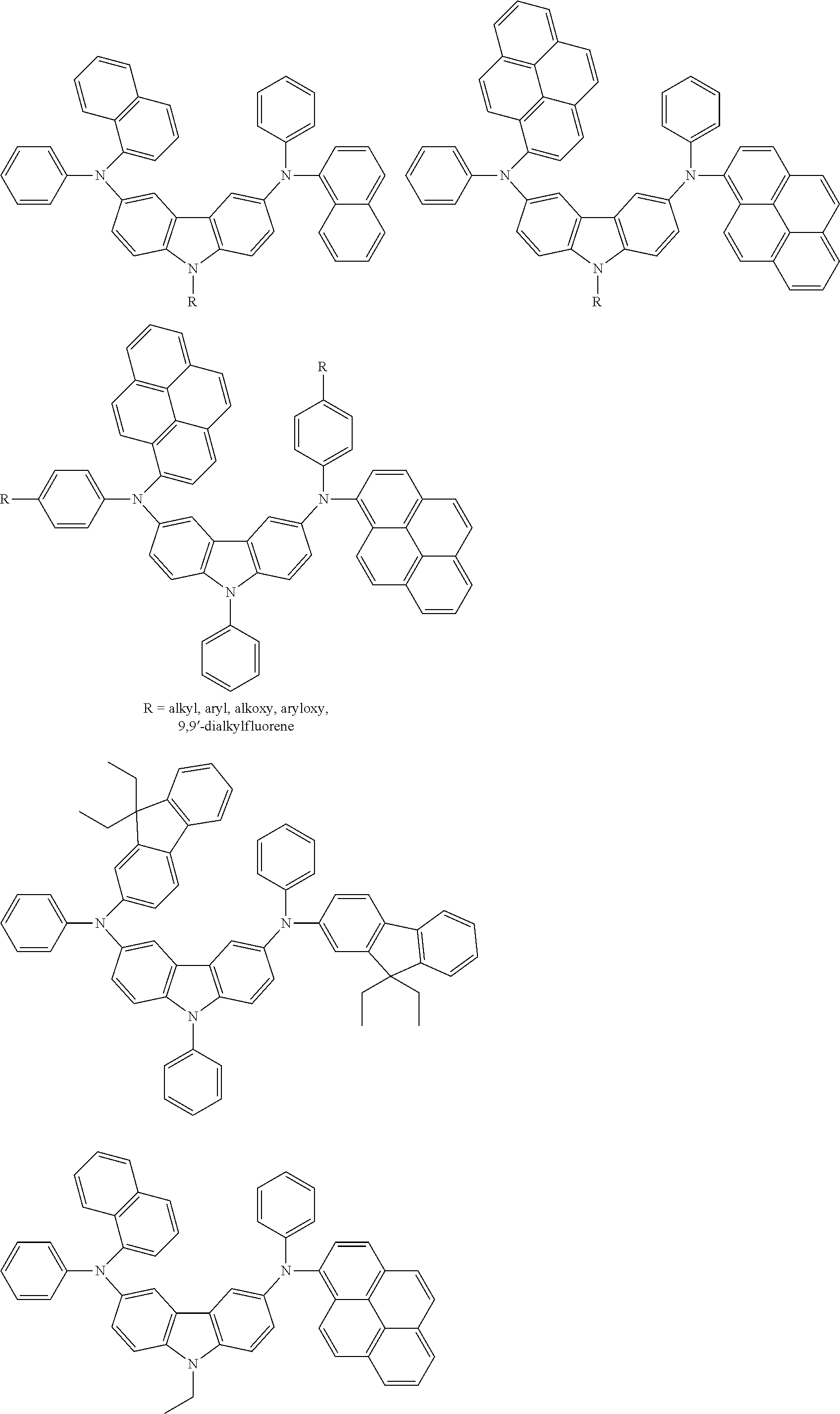

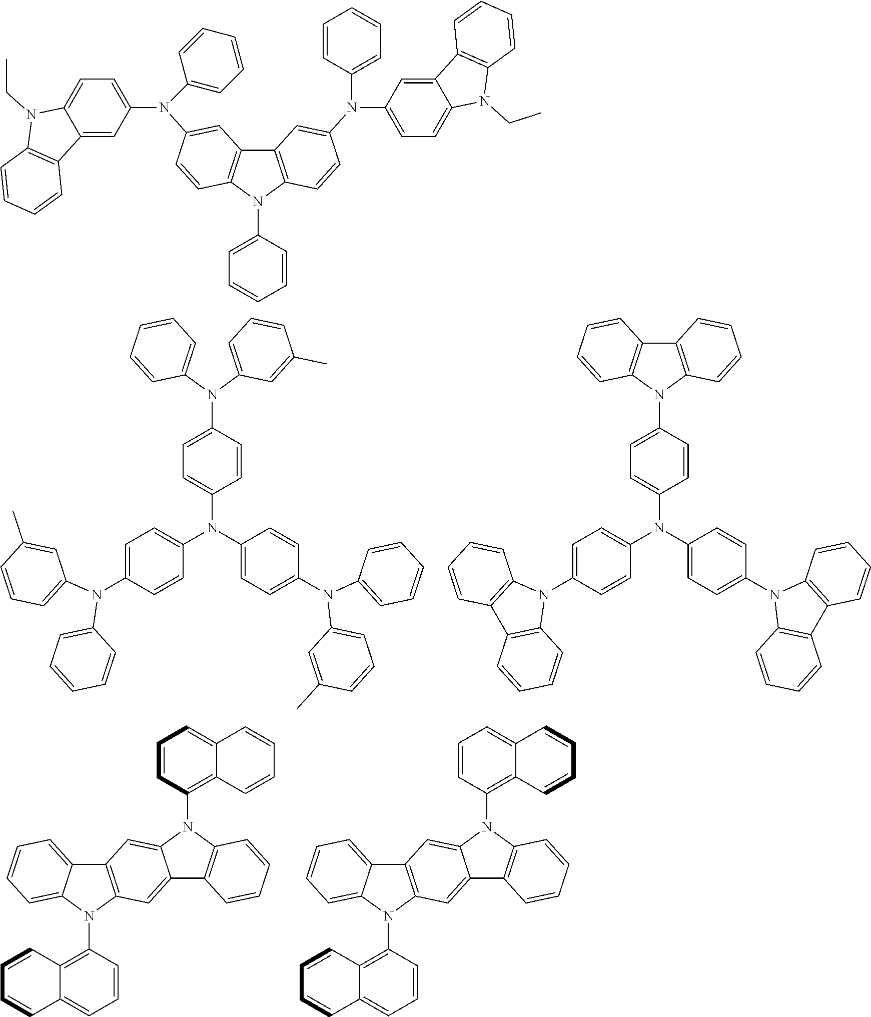

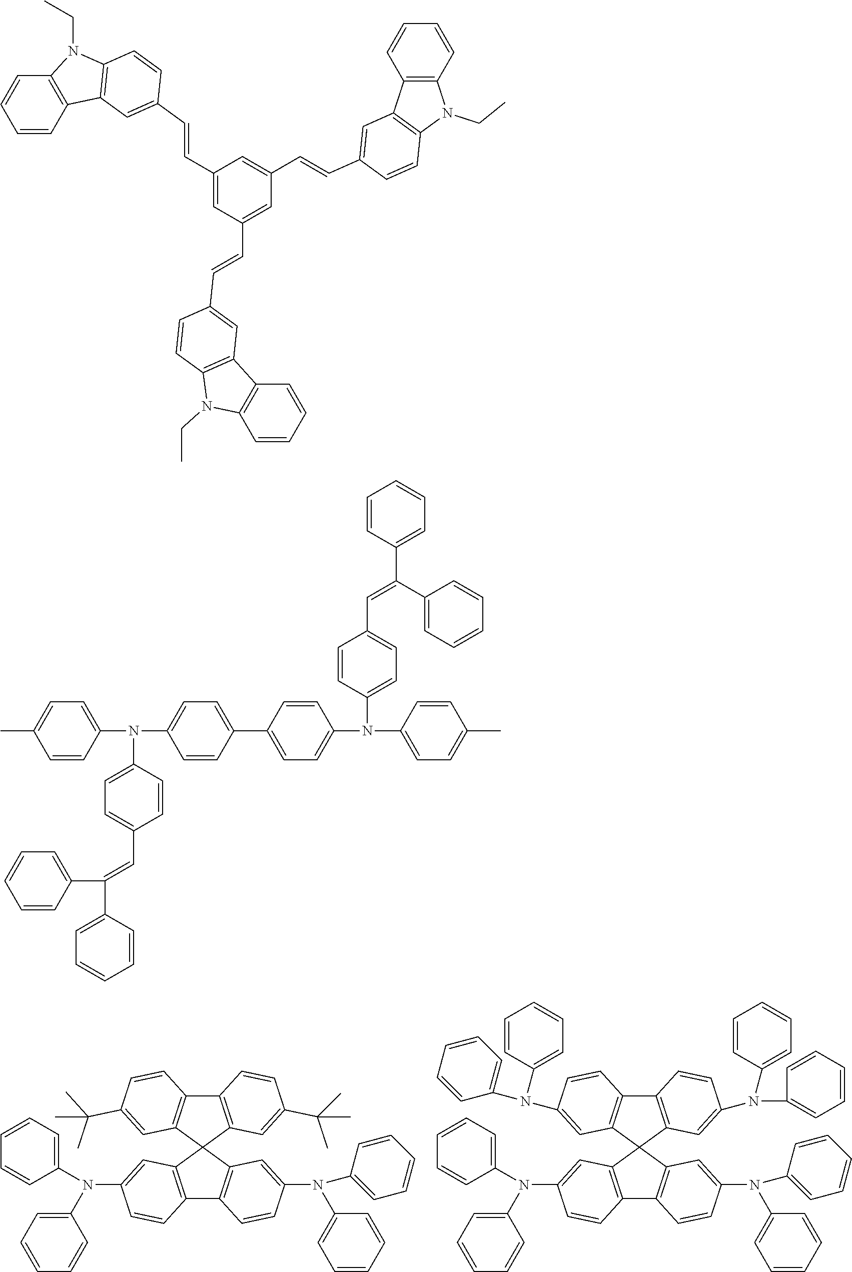

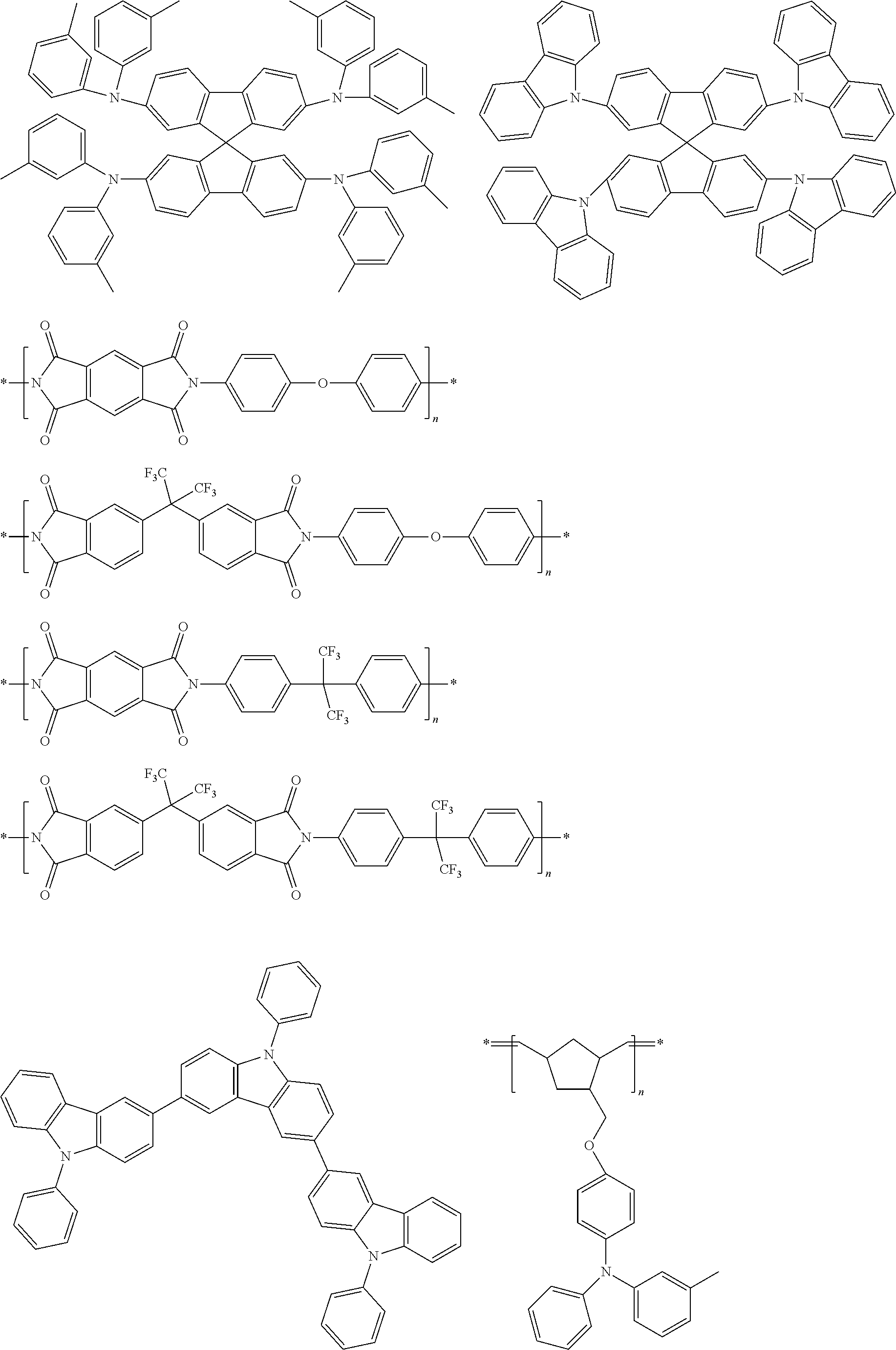

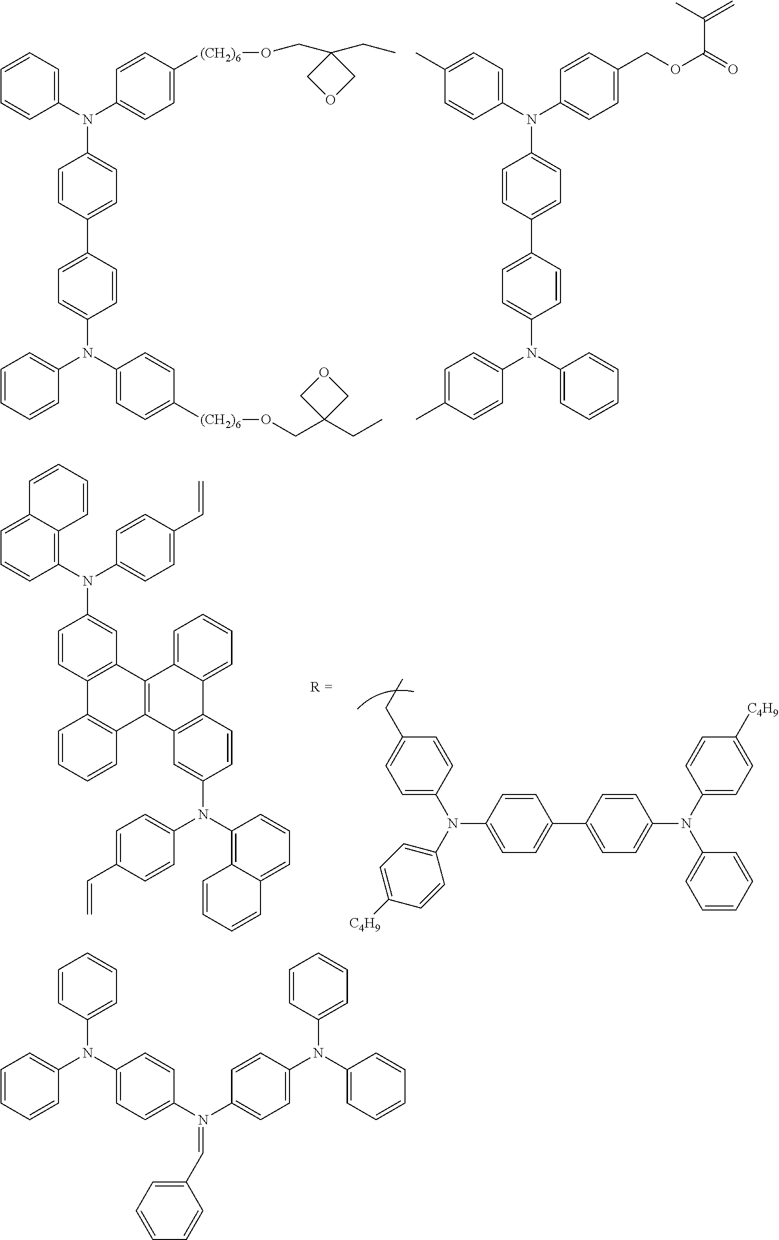

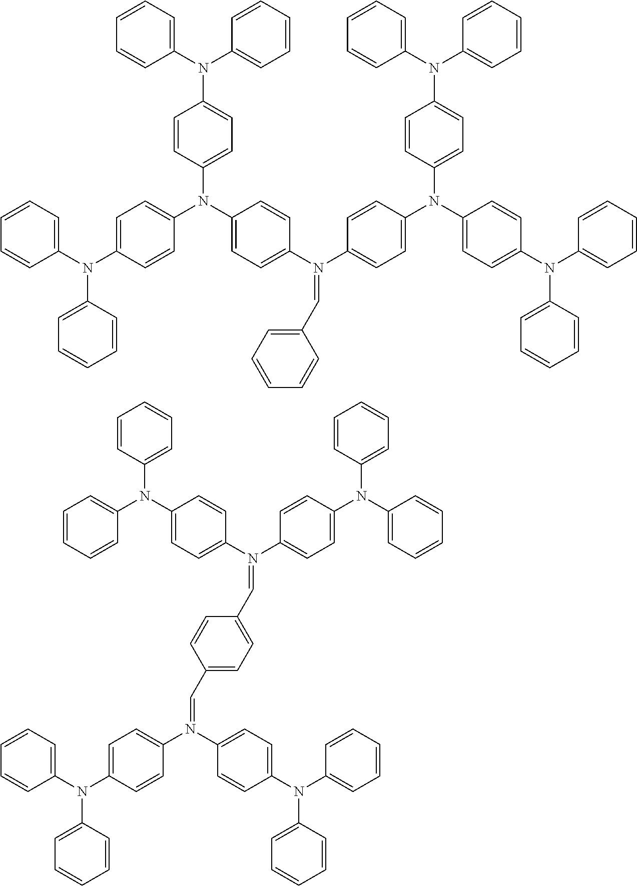

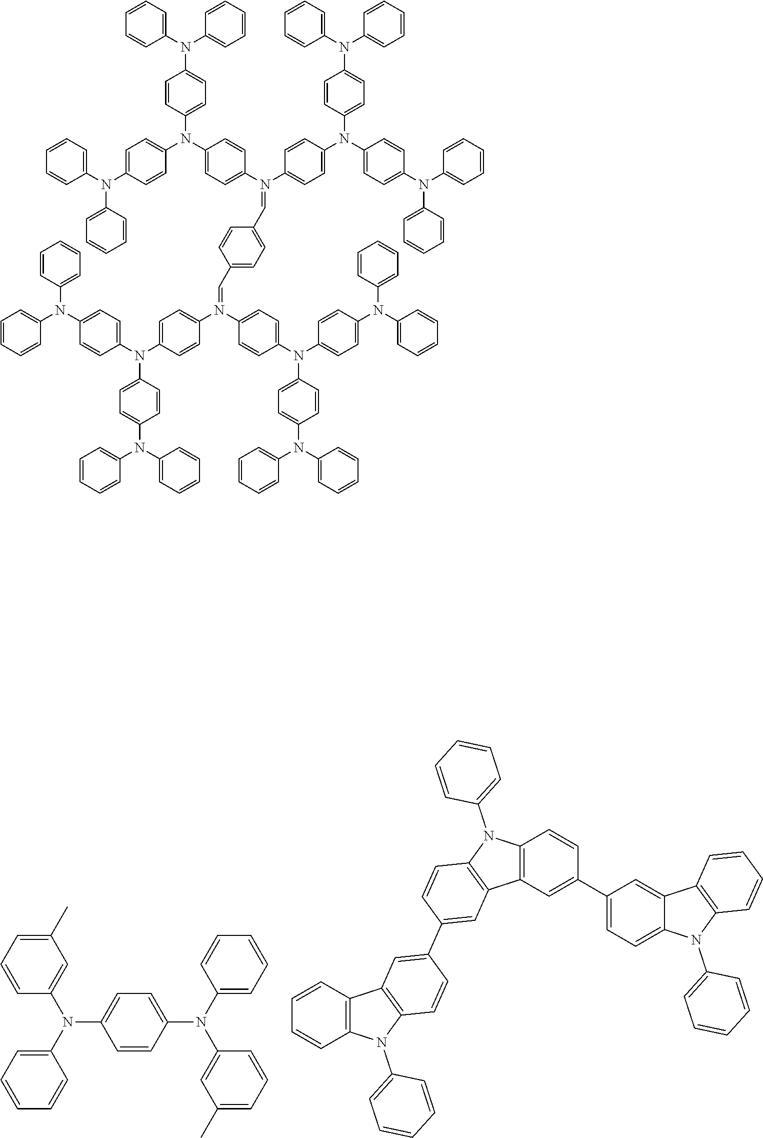

Next, preferred compounds for use as an electron blocking material are mentioned below.

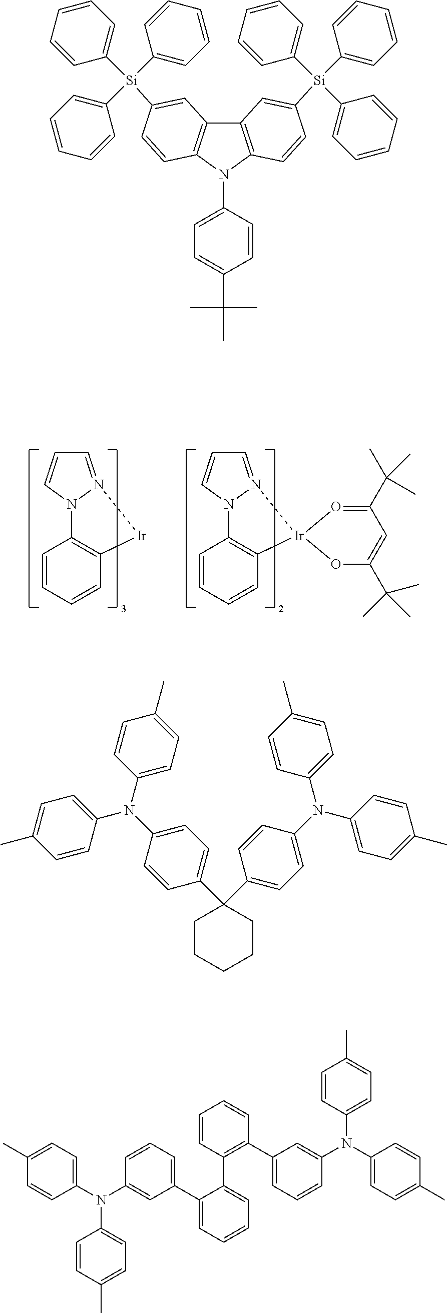

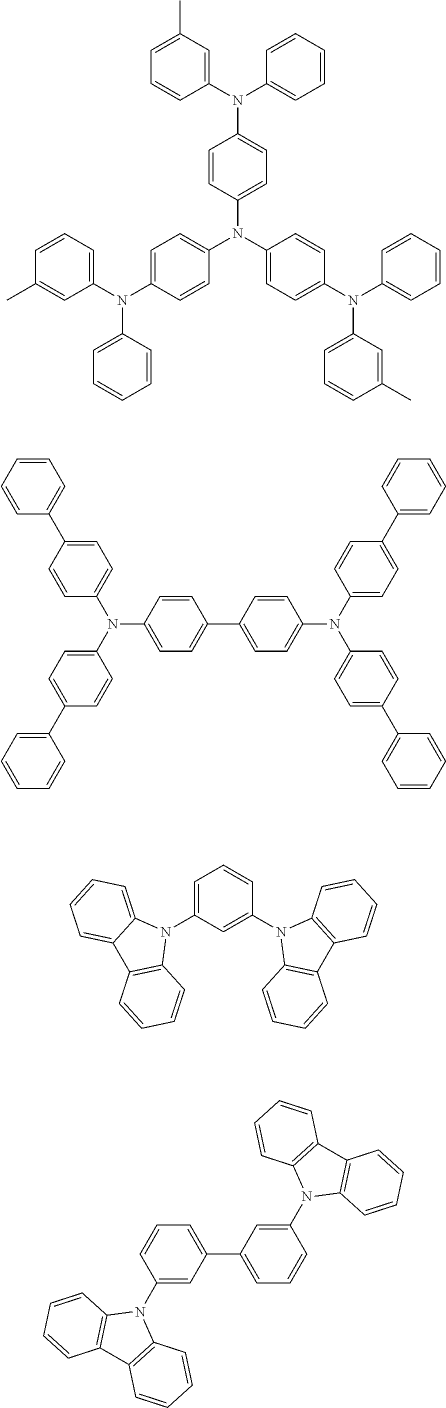 [SMILES: Cc1cccc(N(c2ccccc2)c2ccc(N(c3ccc(N(c4ccccc4)c4cccc(C)c4)cc3)c3ccc(N(c4ccccc4)c4cccc(C)c4)cc3)cc2)c1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1cc(-n2c3ccccc3c3ccccc32)cc(-n2c3ccccc3c3ccccc32)c1 ; c1cc(-c2cccc(-n3c4ccccc4c4ccccc43)c2)cc(-n2c3ccccc3c3ccccc32)c1]

As a hole blocking material, the compounds represented by the general formula (1) are preferably usable. In addition, other preferred compounds for use as a hole blocking material are mentioned below.

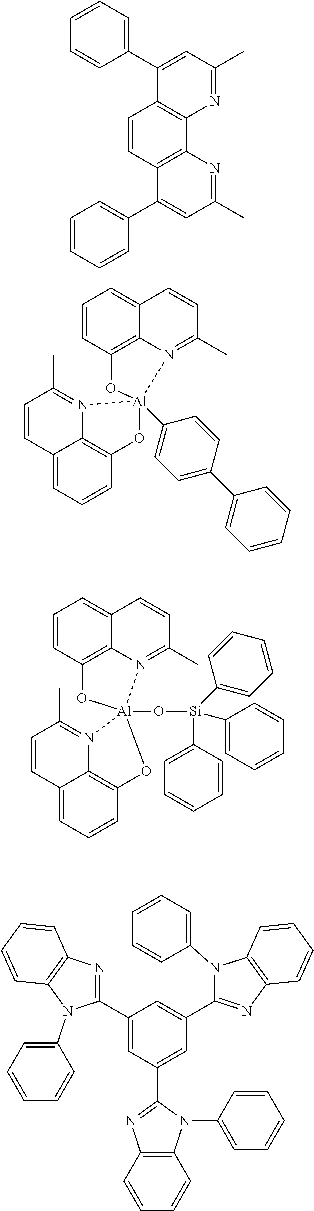

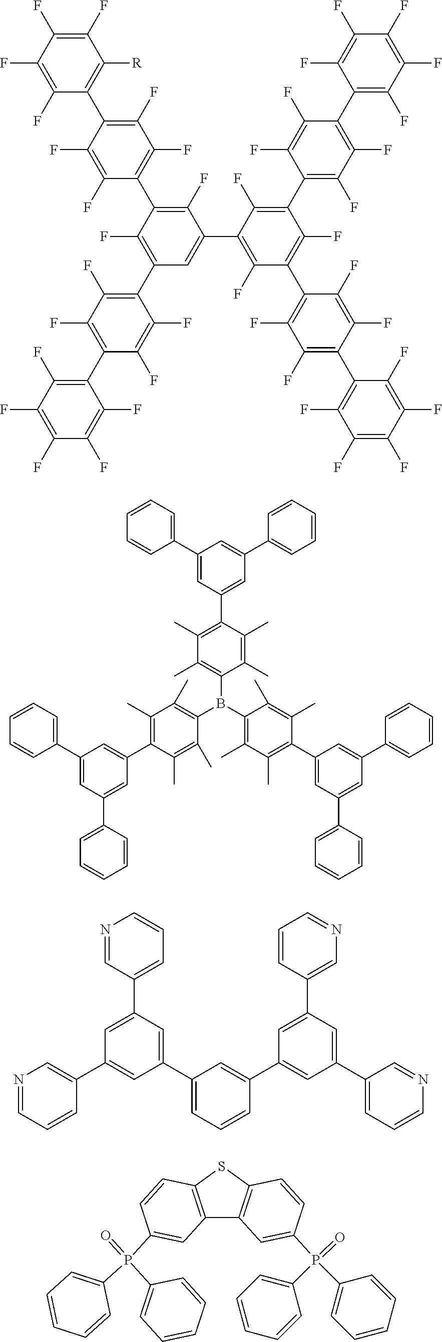

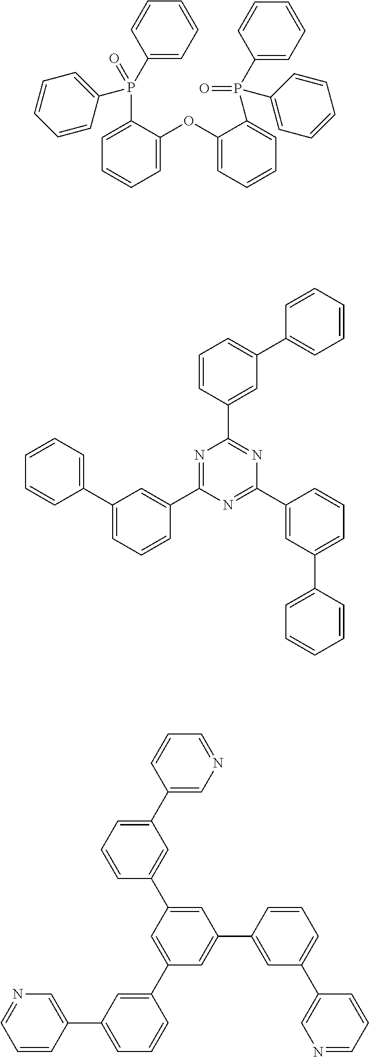

As an electron transport material, the compounds represented by the general formula (1) are preferably usable. In addition, other preferred compounds for use as an electron transport material are mentioned below.

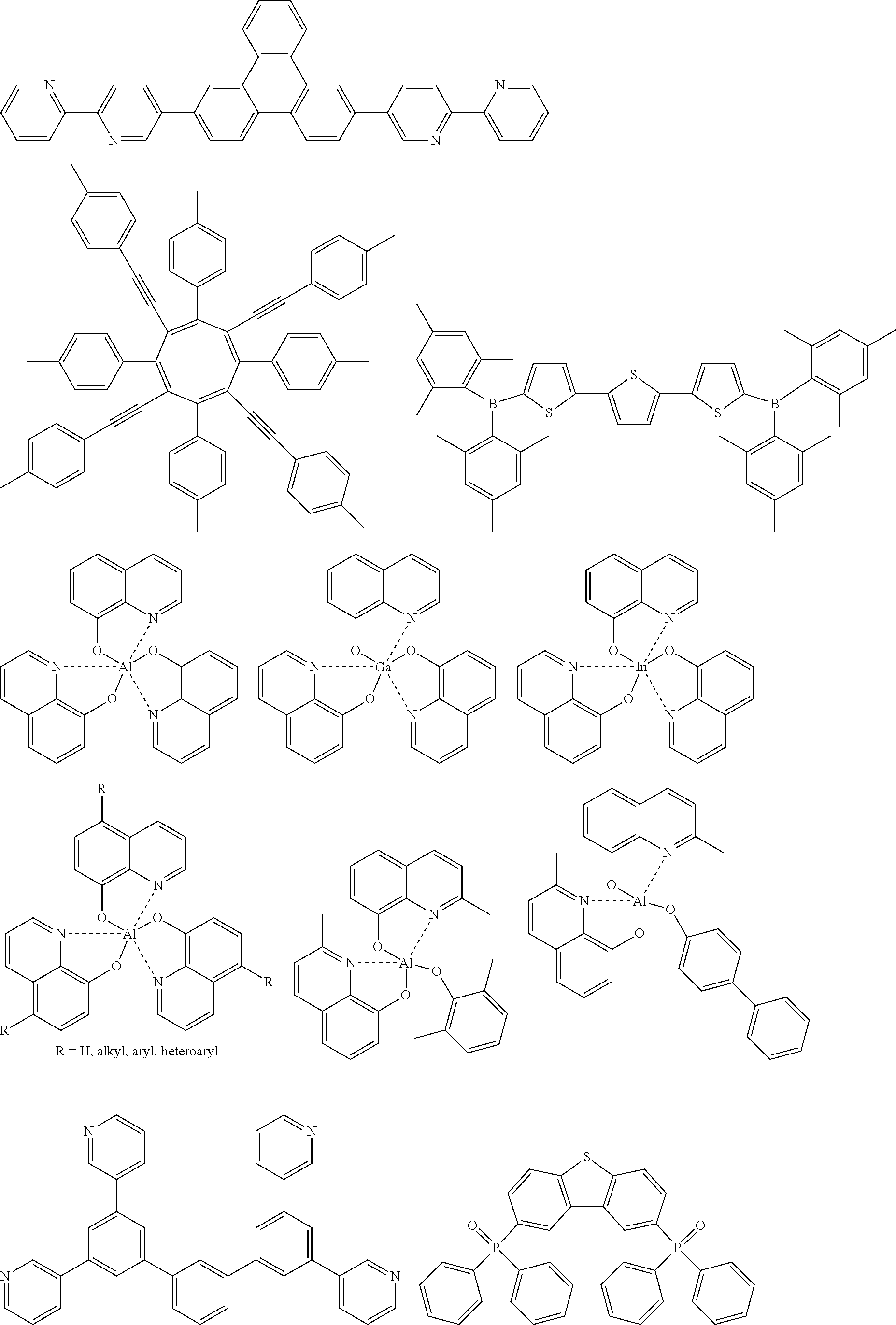

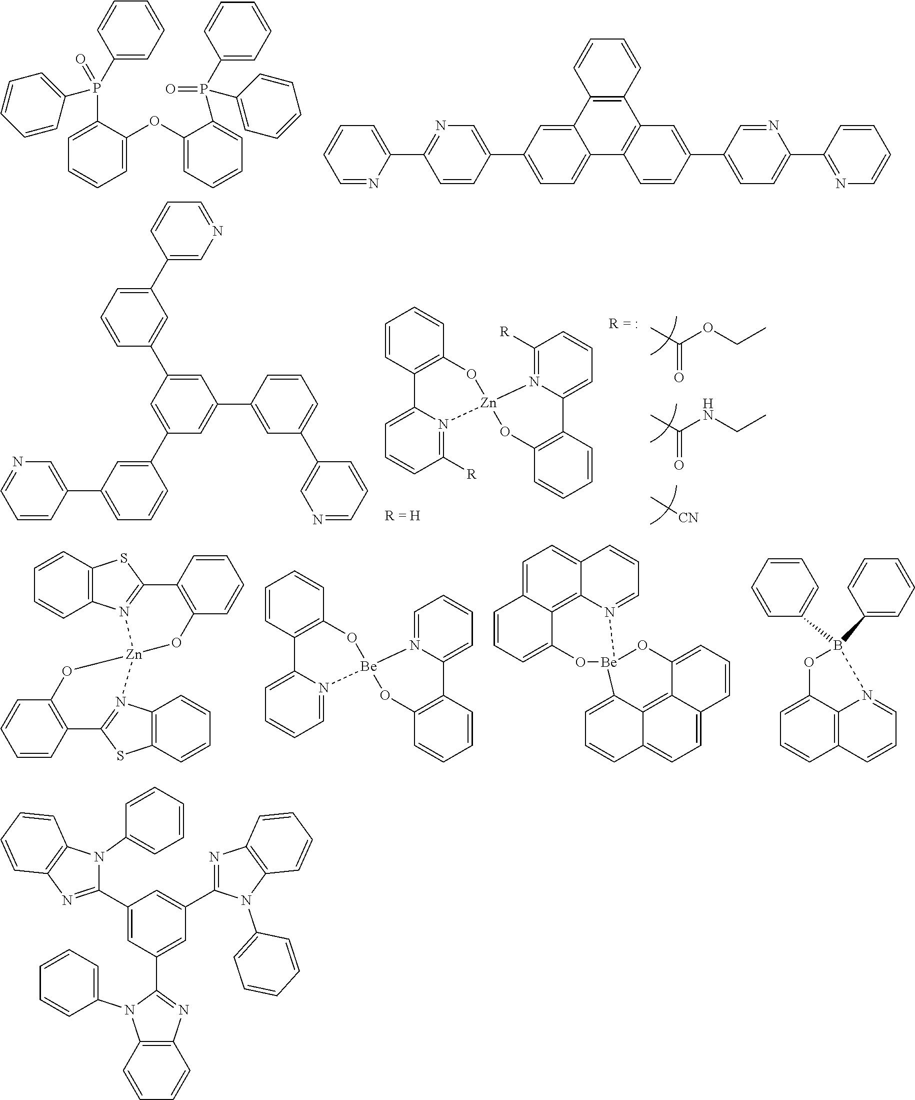

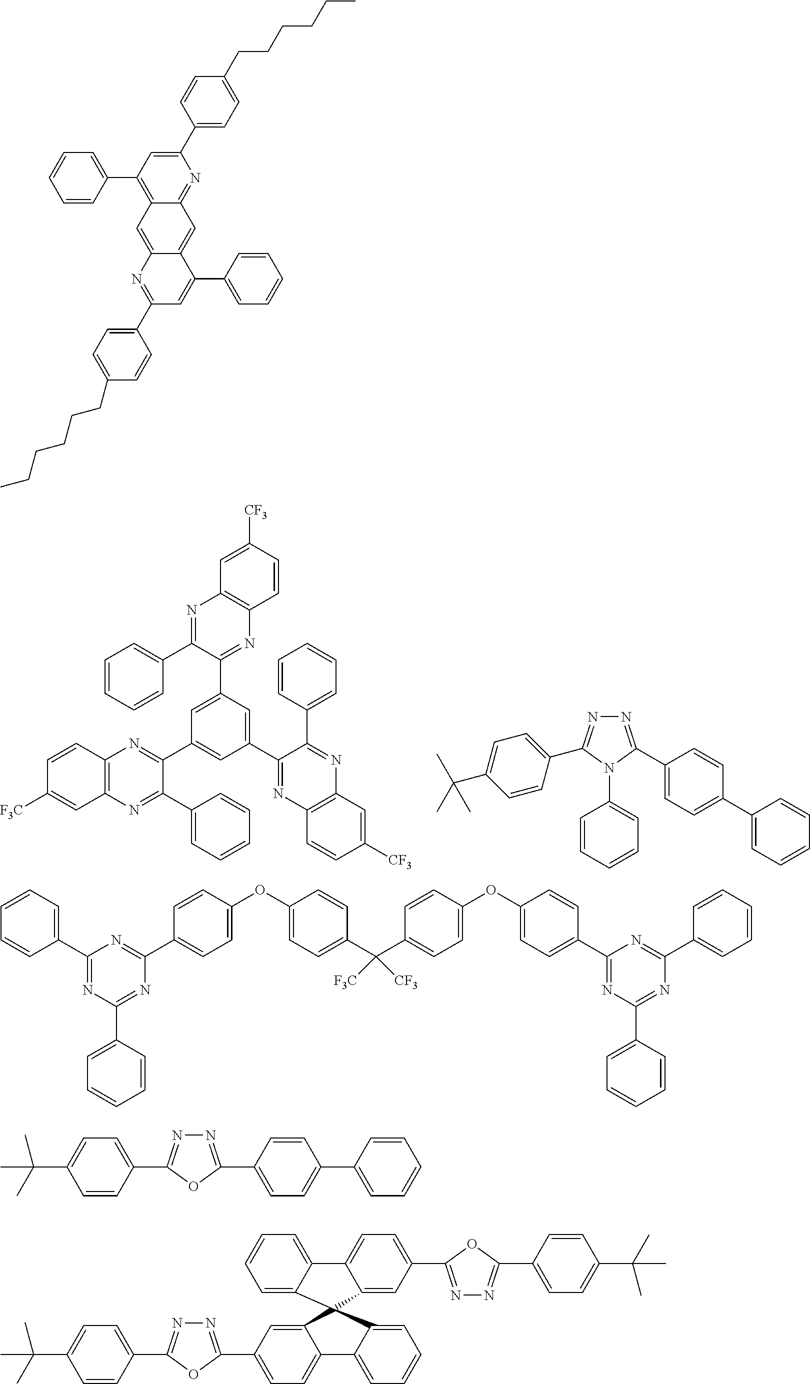

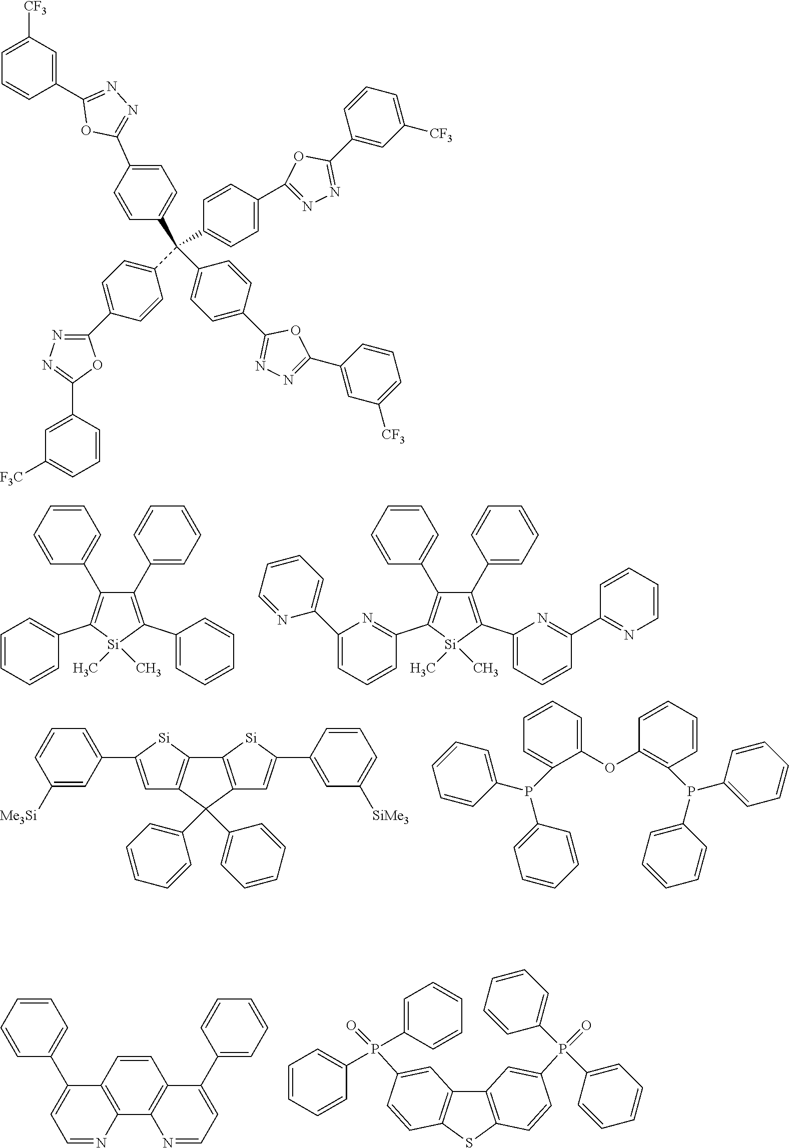

Next, preferred compounds for use as an electron injection material are mentioned below.

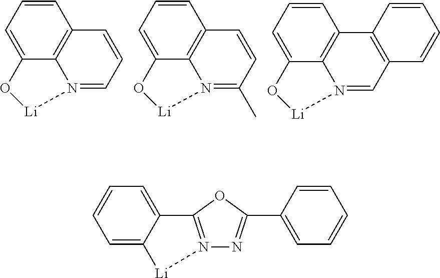

Further, preferred examples of compounds usable as an additive material are mentioned below. For example, the following compounds may be added as a stabilizer material.

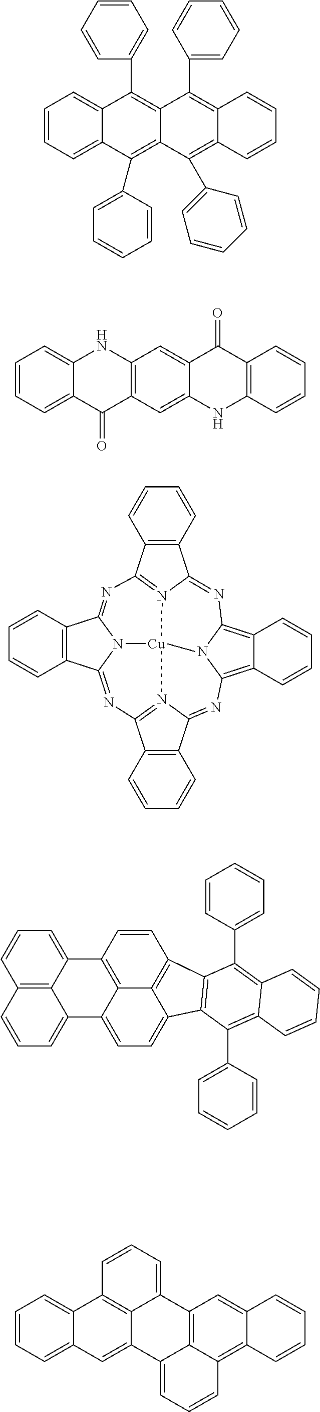

The organic electroluminescent device thus produced by the aforementioned method emits light on application of an electric field between the anode and the cathode of the device. In this case, when the light emission is caused by the excited singlet energy, light having a wavelength that corresponds to the energy level thereof may be confirmed as fluorescent light and delayed fluorescent light. When the light emission is caused by the excited triplet energy, light having a wavelength that corresponds to the energy level thereof may be confirmed as phosphorescent light. The normal fluorescent light has a shorter light emission lifetime than the delayed fluorescent light, and thus the light emission lifetime may be distinguished between the fluorescent light and the delayed fluorescent light.

On the other hand, the phosphorescent light may substantially not be observed with a normal organic compound such as the compound of the present invention at room temperature because the compound immediately deactivates since the excited triplet energy is unstable, the thermal deactivation rate constant is large, and the emission rate constant is small. The excited triplet energy of the normal organic compound may be measured by observing light emission under an extremely low temperature condition.

The organic electroluminescent device of the invention may be applied to any of a single device, a structure with plural devices disposed in an array, and a structure having anodes and cathodes disposed in an X-Y matrix. According to the present invention using the compound represented by the general formula (1) in a light-emitting layer, an organic light-emitting device having a markedly improved light emission efficiency can be obtained. The organic light-emitting device such as the organic electroluminescent device of the present invention may be applied to a further wide range of purposes. For example, an organic electroluminescent display apparatus may be produced with the organic electroluminescent device of the invention, and for the details thereof, reference may be made to S. Tokito, C. Adachi and H. Murata, “Yuki EL Display” (Organic EL Display) (Ohmsha, Ltd.). In particular, the organic electroluminescent device of the invention may be applied to organic electroluminescent illumination and backlight which are highly demanded.

### EXAMPLES

The features of the present invention will be described more specifically with reference to Synthesis Examples and Examples given below. The materials, processes, procedures and the like shown below may be appropriately modified unless they deviate from the substance of the invention. Accordingly, the scope of the invention is not construed as being limited to the specific examples shown below. The light emission characteristics were evaluated using a source meter (2400 Series, produced by Keithley Instruments Inc.), a semiconductor parameter analyzer (E5273A, produced by Agilent Technologies, Inc.), an optical power meter (1930C, produced by Newport Corporation), an optical spectrometer (USB2000, produced by Ocean Optics, Inc.), a spectroradiometer (SR-3, produced by Topcon Corporation), and a streak camera (Model C4334, produced by Hamamatsu Photonics K.K.).

### (Synthesis Example 1) Synthesis of Compound 1

### (1-1) Synthesis of Intermediate A-1

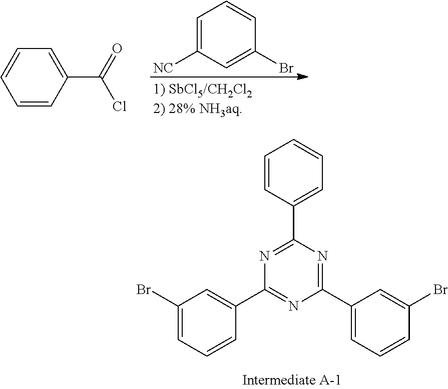 [SMILES: O=C(Cl)c1ccccc1 ; N#Cc1cccc(Br)c1 ; Brc1cccc(-c2nc(-c3ccccc3)nc(-c3cccc(Br)c3)n2)c1]

19 g (0.14 mol) of benzoyl chloride was put into a 1000-mL three-neck flask, which was purged with nitrogen, and then 50 g (0.27 mol) of 3-bromobenzonitrile was added thereto and stirred in a nitrogen stream atmosphere at 0° C. After stirring, 17 mL (0.14 mol) of antimony chloride was added, this was gradually restored from 0° C. to room temperature, and stirred at 60° C. for 1 hour. After stirring, the mixture was cooled, and 400 ml of aqueous ammonia was added and stirred at 0° C. The mixture was filtered under suction to give a solid. The resultant solid was washed with water and methanol in that order. After washing, the solid was transferred into an eggplant flask, 200 mL of N,N-dimethylformamide was added thereto and stirred at 153° C. After stirring, the mixture was filtered under suction. The filtrate was again transferred into an eggplant flask, and 100 ml of N,N-dimethylformamide was added and stirred at 153° C. After stirring, the mixture was again filtered under suction. The resultant filtrate and the precipitated solid from the filtrate were put into an eggplant flask, and evaporated under reduced pressure to thereby reduce N,N-dimethylformamide to about 100 mL. 500 mL of water was added to the mixture, stirred and filtered. The resultant solid was washed with water. The solid was added to 500 mL of methanol, irradiated with ultrasonic waves, and then filtered under suction to give a white powdery solid of the intended product (intermediate A-1: 2,4-bis(3-bromophenyl)-6-phenyl-1,3,5-triazine) at a production quantity of 4.2 g and a yield of 66%.

¹H NMR (500 Hz, CDCl₃, δ): 8.88 (t, J=1.8 Hz, 2H), 8.77-8.75 (m, 2H), 8.71-8.69 (m, 2H), 7.76-7.74 (m, 2H), 7.66-7.58 (m, 3H), 7.47 (t, J=7.8 Hz, 2H)

MS: 470.22

### (1-2) Synthesis of Compound 1

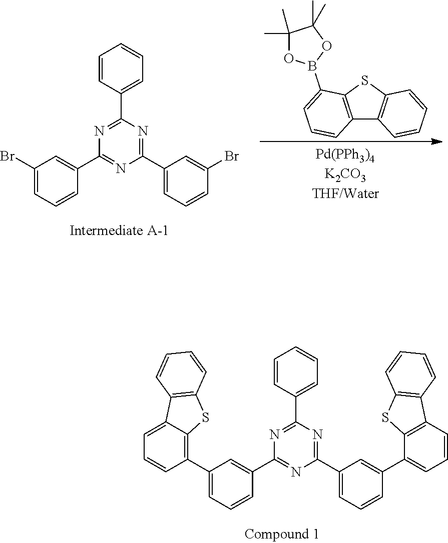 [SMILES: Brc1cccc(-c2nc(-c3ccccc3)nc(-c3cccc(Br)c3)n2)c1 ; CC1(C)OB(c2cccc3c2sc2ccccc23)OC1(C)C ; c1ccc(-c2nc(-c3cccc(-c4cccc5c4sc4ccccc45)c3)nc(-c3cccc(-c4cccc5c4sc4ccccc45)c3)n2)cc1]

1.1 g (2.4 mmol) of the intermediate A-1 (2,4-bis(3-bromophenyl)-6-phenyl-1,3,5-triazine), 1.8 g (5.8 mmol) of 2-(dibenzo[b,d]thiophen-4-yl)4,4,5,5-tetramethyl-1,3,2-dioxabororan, 0.080 g 80.069 mmol) of tetrakis(triphenylphosphine)palladium(0), and 11 g (80 mmol) of potassium carbonate were put into a 200-mL three-neck flask, which was then purged with nitrogen. 120 mL of tetrahydrofuran and 40 mL of water were added to the mixture, and stirred in a nitrogen atmosphere at 60° C. for 20 hours. After stirring, the mixture was filtered under suction to give a solid. The resultant solid was washed with water and acetone in that order to give a powdery white solid of the intended product (compound 1) at a production quantity of 1.6 g and a yield of 82%.

¹H NMR (500 Hz, CDCl₃, δ): 9.24 (s, 2H), 8.87 (d, J=7.8 Hz, 2H), 8.81 (d, J=7.0 Hz, 2H), 8.21 (d, J=7.9 Hz, 4H), 7.99 (d, J=7.3 Hz, 2H), 7.78 (d, J=7.7 Hz, 2H), 7.74 (t, J=7.8 Hz, 2H), 7.64-7.55 (m, 7H), 7.51-7.44 (m, 4H)

MS: 673.45

### (Synthesis Example 2) Synthesis of Compound 1 in Other Synthesis Route

### (2-1) Synthesis of Intermediate D-1

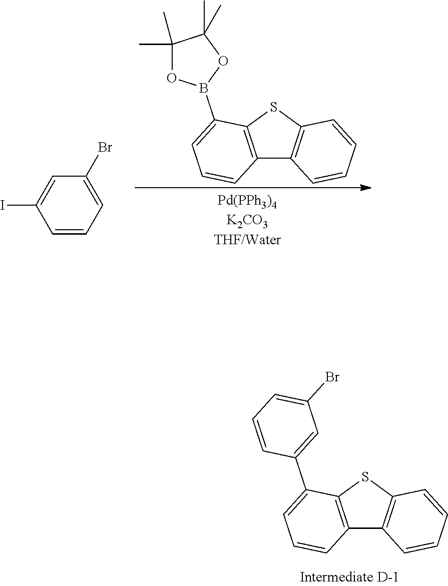 [SMILES: Brc1cccc(I)c1 ; CC1(C)OB(c2cccc3c2sc2ccccc23)OC1(C)C ; Brc1cccc(-c2cccc3c2sc2ccccc23)c1]

24 g (85 mmol) of 1-bromo-3-iodobenzene, 24 g (77 mmol) of 2-(dibenzo[b,d]thiophen-4-yl)-4,4,5,5-tetramethyl-1,3,2-dioxabororan, 2.7 g (2.3 mmol) of tetrakis(triphenylphosphine)palladium(0), and 28 g (0.20 mol) of potassium carbonate were put into a 1000-mL three-neck flask, which was then purged with nitrogen. 400 mL of tetrahydrofuran and 100 mL of water were added to the mixture, and stirred in a nitrogen atmosphere at 80° C. for 12 hours. After stirring, the mixture was put into 300 mL of chloroform, and washed with water added thereto. After washing, the organic layer and the aqueous layer were separated, and the organic layer was filtered under suction through Celite and silica gel to give a filtrate. The resultant filtrate was concentrated and purified through silica gel column chromatography. At this time, hexane was used as the developing solvent. The resultant fraction was concentrated to give a solid, which was recrystallized with a mixed solvent of chloroform and hexane to give a powdery white solid of the intended product (intermediate D-1: 4-(3-bromophenyl)dibenzo[b,d]thiophene) at a production quantity of 24 g and a yield of 90%.

¹H NMR (500 Hz, CDCl₃, δ): 8.20-8.17 (m, 2H), 7.88 (t, J=1.8 Hz, 1H), 7.85-7.83 (m, 1H), 7.70-7.68 (m, 1H), 7.58-7.54 (m, 2H), 7.50-7.41 (m, 3H), 7.38 (t, J=7.9 Hz, 1H)

MS: 339.67

### (2-2) Synthesis of Intermediate D-2

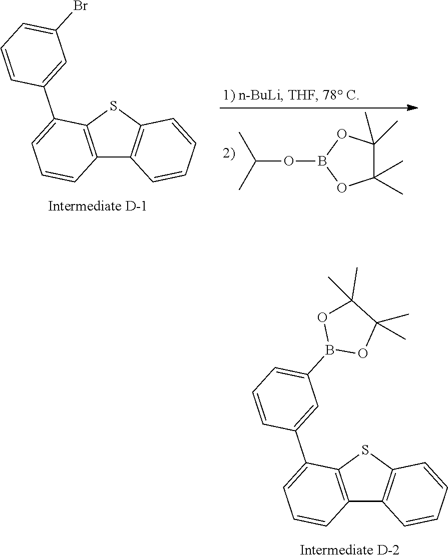

26 g (77 mmol) of the intermediate D-1 (4-(3-bromophenyl)dibenzo[b,d]thiophene) was put into a 1000-mL flasks, which was then purged with nitrogen, and 500 mL of tetrahydrofuran was added thereto and stirred in a nitrogen atmosphere at −78° C. for 1 hour. To this solution, added was 32 mL (81 mmol) of 2.5 mol/L n-butyllithium/hexane solution, and the resultant solution was stirred at −78° C. for 1 hour. After stirring, 16 g (84 mmol) of 2-isopropoxy-4,4,5,5-tetramethyl-1,3,2-dioxaborolane was added to the solution, then gradually restored from −78° C. to room temperature, and stirred at room temperature for 12 hours. After stirring, 100 mL of water and 300 mL of chloroform were added to the solution and stirred. After stirring, the aqueous layer and the organic layer were separated, and the organic layer was washed with saturated saline water. After washing, the organic layer was dried with magnesium sulfate added thereto. After drying, the mixture was filtered under suction to give a filtrate. The resultant filtrate was concentrated and purified through silica gel column chromatography. At this time, a mixed solvent of chloroform/hexane=½ was used as the developing solvent. The resultant fraction was concentrated to give a yellow liquid of the intended product (intermediate D-2: 2-[3-(dibenzo[b,d]thiophen-4-yl)phenyl]-4,4,5,5-tetramethyl-1,3,2-dioxaborolane) at a production quantity of 15 g and a yield of 52%.

¹H NMR (500 Hz, CDCl₃, δ): 8.20-8.18 (m, 1H), 8.15 (dd, J=7.5 Hz, 1.5 Hz, 1H), 8.12 (s, 1H), 7.90-7.88 (m, 2H), 7.84-7.83 (m, 1H), 7.56-7.51 (m, 3H), 7.47-7.45 (m, 2H), 1.37 (s, 12H)

MS: 386.34

### (2-3) Synthesis of Compound 1

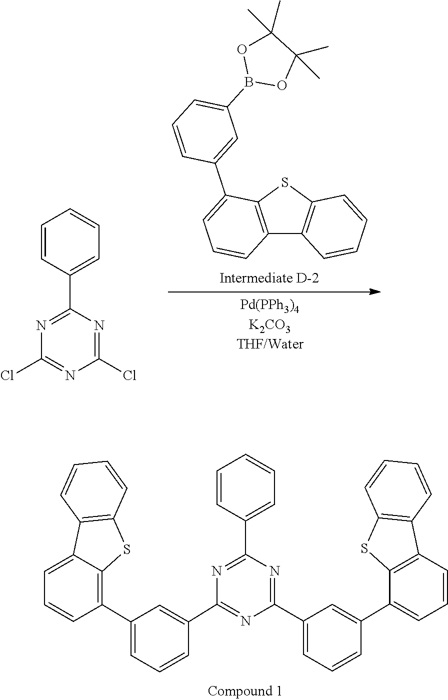 [SMILES: Clc1nc(Cl)nc(-c2ccccc2)n1 ; CC1(C)OB(c2cccc(-c3cccc4c3sc3ccccc34)c2)OC1(C)C ; c1ccc(-c2nc(-c3cccc(-c4cccc5c4sc4ccccc45)c3)nc(-c3cccc(-c4cccc5c4sc4ccccc45)c3)n2)cc1]

0.67 g (3.0 mmol) of 2,4-dichloro-6-phenyl-1,3,5-triazine, 2.8 g (7.1 mmol) of the intermediate D-2 (2-[3-(dibenzo[b,d]thiophen-4-yl)phenyl]-4,4,5,5-tetramethyl-1,3,2-dioxaborolane), 0.01 g (0.087 mmol) of tetrakis(triphenylphosphine)palladium(0), and 5.5 g (40 mmol) of potassium carbonate were put into a 200-mL three-neck flask, which was then purged with nitrogen. 60 mL of tetrahydrofuran and 20 mL of water were added to the mixture, and stirred in a nitrogen atmosphere at 95° C. for 24 hours. After stirring, the mixture was filtered under suction to give a solid. The resultant solid was washed with water and acetone in that order to give a powdery white solid of the intended product (compound 1) at a production quantity of 1.6 g and a yield of 80%.

¹H NMR (500 Hz, CDCl₃, δ): 9.24 (s, 2H), 8.87 (d, J=7.8 Hz, 2H), 8.81 (d, J=7.0 Hz, 2H), 8.21 (d, J=7.9 Hz, 4H), 7.99 (d, J=7.3 Hz, 2H), 7.78 (d, J=7.7 Hz, 2H), 7.74 (t, J=7.8 Hz, 2H), 7.64-7.55 (m, 7H), 7.51-7.44 (m, 4H)

MS: 673.45

### (Synthesis Example 3) Synthesis of Compound 2

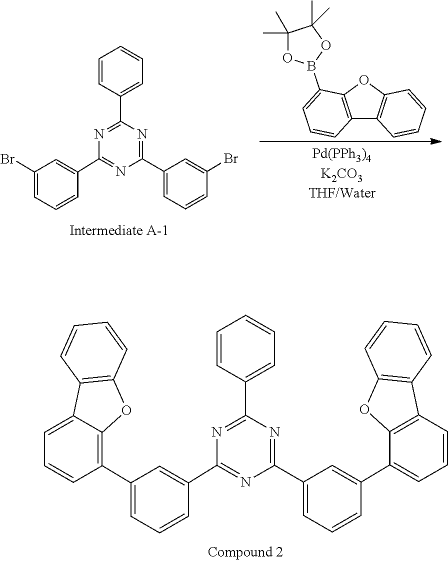 [SMILES: Brc1cccc(-c2nc(-c3ccccc3)nc(-c3cccc(Br)c3)n2)c1 ; CC1(C)OB(c2cccc3c2oc2ccccc23)OC1(C)C ; c1ccc(-c2nc(-c3cccc(-c4cccc5c4oc4ccccc45)c3)nc(-c3cccc(-c4cccc5c4oc4ccccc45)c3)n2)cc1]

1.5 g (3.1 mmol) of the intermediate A-1 (2,4-bis(3-bromophenyl)-6-phenyl-1,3,5-triazine) synthesized in the same manner as in Synthesis Example 1, 2.2 g (7.5 mmol) of 2-(dibenzo[b,d]furan-4-yl)-4,4,5,5-tetramethyl-1,3,2-dioxaborolane, 0.10 g (0.087 mmol) of tetrakis(triphenylphosphine)palladium(0), and 5.5 g (40 mmol) of potassium carbonate were put into a 200-mL three-neck flask, which was then purged with nitrogen. 60 mL of tetrahydrofuran and 20 mL of water were added to the mixture, and stirred in a nitrogen atmosphere at 60° C. for 20 hours. After stirring, the mixture was added to 200 mL of toluene, and washed with water added thereto. After washing, the organic layer and the aqueous layer were separated, and the organic layer was filtered under suction through Celite and silica gel to give a filtrate. The resultant filtrate was concentrated to give a solid, which was recrystallized with a mixed solvent of chloroform and methanol to give a powdery white solid of the intended product (compound 2) at a production quantity of 1.6 g and a yield of 80%.

¹H NMR (500 Hz, CDCl₃, δ): 9.45 (s, 2H), 8.88 (t, J=8.1 Hz, 4H), 8.20 (d, J=7.6 Hz, 2H), 8.01-7.97 (m, 4H), 7.78-7.75 (m, 4H), 7.64-7.58 (m, 5H), 7.47-7.26 (m, 6H)

MS: 641.62

### (Synthesis Example 4) Synthesis of Compound 2 in Other Synthesis Route

### (4-1) Synthesis of Intermediate D-3

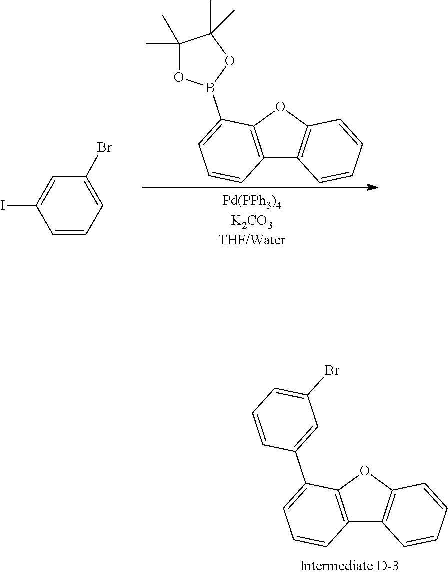 [SMILES: Brc1cccc(I)c1 ; CC1(C)OB(c2cccc3c2oc2ccccc23)OC1(C)C ; Brc1cccc(-c2cccc3c2oc2ccccc23)c1]

4.0 g (14 mmol) of 1-bromo-3-iodobenzene, 4.2 g (14 mmol) of 2-(dibenzo[b,d]furan-4-yl)-4,4,5,5-tetramethyl-1,3,2-dioxaborolane, 0.50 g (0.43 mmol) of tetrakis(triphenylphosphine)palladium(0), and 3.3 g (24 mmol) of potassium carbonate were put into a 200-mL flask, which was then purged with nitrogen. 40 mL of tetrahydrofuran and 12 mL of water were added to the mixture, and stirred in a nitrogen atmosphere at 80° C. for 24 hours. After stirring, the mixture was added to chloroform, and washed with water added thereto. After washing, the organic layer and the aqueous layer were separated, and the organic layer was filtered under suction through Celite and silica gel to give a filtrate. The resultant filtrate was concentrated and purified through silica gel column chromatography. At this time, a mixed solvent of chloroform/hexane=¼ was used as the developing solvent. The resultant fraction was concentrated to give a powder white solid of the intended product (intermediate D-3: 4-(3-bromophenyl)dibenzo[b,d]furan) at a production quantity of 4.0 g and a yield of 88%.

¹H NMR (500 Hz, CDCl₃, δ): 8.06 (t, J=1.8 Hz, 1H), 7.99 (dd, J=7.7 Hz, 1.0 Hz, 1H), 7.96 (dd, J=7.7 Hz, 1.2 Hz, 1H), 7.87-7.85 (m, 1H), 7.62 (d, J=8.2 Hz, 1H), 7.58-7.55 (m, 2H), 7.49 (td, J=8.0 Hz, 1.8 Hz 1H), 7.45-7.26 (m, 3H)

MS: 324.12

### (4-2) Synthesis of Intermediate D-4

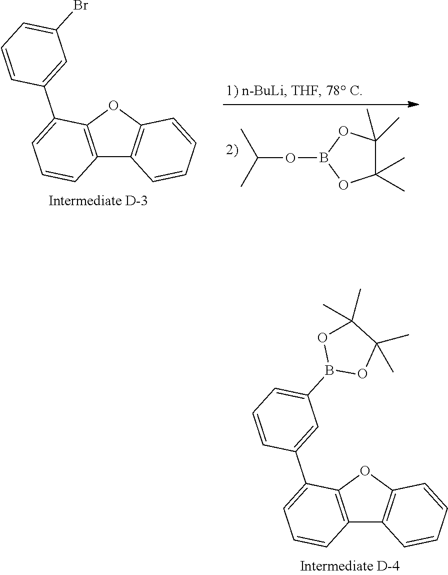 [SMILES: Brc1cccc(-c2cccc3c2oc2ccccc23)c1 ; CC(C)OB1OC(C)(C)C(C)(C)O1 ; CC1(C)OB(c2cccc(-c3cccc4c3oc3ccccc34)c2)OC1(C)C]

3.8 g (12 mmol) of the intermediate D-3 (4-(3-bromophenyl)dibenzo[b,d]furan) was put into a 200-mL three-neck flask, which was then purged with nitrogen, and 50 mL of tetrahydrofuran was added thereto, and stirred in a nitrogen atmosphere at −78° C. for 1 hour. To the solution, added was 4.9 mL (12 mmol) of 2.5 mol/L n-butyllithium/hexane solution, and the solution was stirred at −78° C. for 1 hour. After stirring, 2.4 g (13 mmol) of 2-isopropoxy-4,4,5,5-tetramethyl-1,3,2-dioxaborolane was added to the solution, then gradually restored from −78° C. to room temperature, and stirred at room temperature for 12 hours. After stirring, 100 mL of water and 100 mL of chloroform were added to the solution, and stirred. After stirring, the aqueous layer and the organic layer were separated, and the organic layer was washed with saturated saline water. After washing, the organic layer was dried with magnesium sulfate added thereto. After drying the mixture was filtered under suction to give a filtrate. The resultant filtrate was concentrated, and purified through silica gel column chromatography. At this time, a mixed solvent of chloroform/hexane=½ was used as the developing solvent. The resultant fraction was concentrated to give a transparent liquid of the intended product (intermediate D-4: 2-[3-(dibenzo[b,d]furan-4-yl)phenyl]-4,4,5,5-tetramethyl-1,3,2-dioxaborolane) at a production quantity of 2.8 g and a yield of 64%.

### (4-3) Synthesis of Compound 2

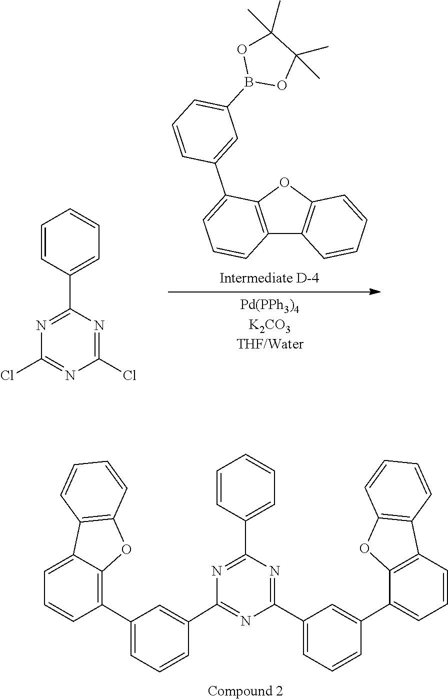 [SMILES: Clc1nc(Cl)nc(-c2ccccc2)n1 ; CC1(C)OB(c2cccc(-c3cccc4c3oc3ccccc34)c2)OC1(C)C ; c1ccc(-c2nc(-c3cccc(-c4cccc5c4oc4ccccc45)c3)nc(-c3cccc(-c4cccc5c4oc4ccccc45)c3)n2)cc1]

0.70 g (3.1 mmol) of 2,4-dichloro-6-phenyl-1,3,5-triazine, 2.8 g (7.4 mmol) of the intermediate D-4 (2-[3-(dibenzo[b,d]furan-4-yl)phenyl]-4,4,5,5-tetramethyl-1.3,2-dioxaborolane), 0.10 g (0.087 mmol) of tetrakis(triphenylphosphine)palladium(0), and 5.5 g (40 mmol) of potassium carbonate were put into a 200-mL three-neck flask, which was then purged with nitrogen. 60 mL of tetrahydrofuran and 20 mL of water were added to the mixture, and stirred in a nitrogen atmosphere at 95° C. for 24 hours. After stirring, the mixture was filtered under suction to give a solid. The resultant solid was washed with water and acetone in that order to give a powdery white solid of the intended product (compound 2) at a production quantity of 1.5 g and a yield of 75%.

¹H NMR (500 Hz, CDCl₃, δ): 9.45 (s, 2H), 8.88 (t, J=8.1 Hz, 4H), 8.20 (d, J=7.6 Hz, 2H), 8.01-7.97 (m, 4H), 7.78-7.75 (m, 4H), 7.64-7.58 (m, 5H), 7.47-7.26 (m, 6H)

MS: 641.62

### (Synthesis Example 5) Synthesis of Compound 3

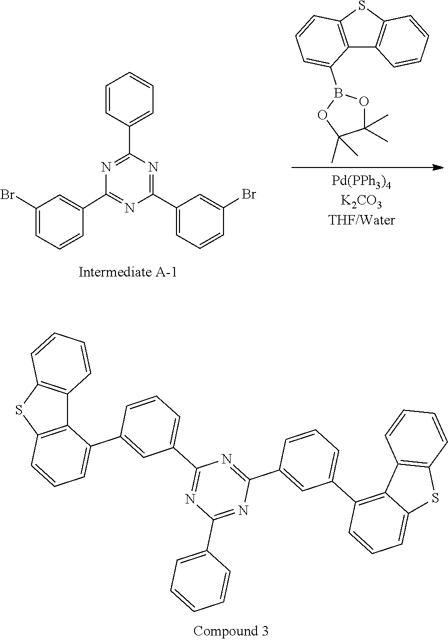 [SMILES: Brc1cccc(-c2nc(-c3ccccc3)nc(-c3cccc(Br)c3)n2)c1 ; CC1(C)OB(c2cccc3sc4ccccc4c23)OC1(C)C ; c1ccc(-c2nc(-c3cccc(-c4cccc5sc6ccccc6c45)c3)nc(-c3cccc(-c4cccc5sc6ccccc6c45)c3)n2)cc1]

1.0 g (2.1 mmol) of the intermediate A-1 (2,4-bis(3-bromophenyl)-6-phenyl-1,3,5-triazine) synthesized in the same manner as in Synthesis Example 1, 1.6 g (5.2 mmol) of 2-(dibenzo[b,d]thiophen-4-yl)-4,4,5,5-tetramethyl-1,3,2-dioxaborolane, 0.25 g (0.21 mmol) of tetrakis(triphenylphosphine)palladium(0), and 5.5 g (40 mmol) of potassium carbonate were put into a 200-mL three-neck flask, which was then purged with nitrogen. 60 mL of tetrahydrofuran and 20 mL of water were added to the mixture, and stirred in a nitrogen atmosphere at 95° C. for 24 hours. After stirring, the mixture was added to 100 mL of toluene, and washed with water added thereto. After washing, the organic layer and the aqueous layer were separated, and the organic layer was filtered under suction through Celite and silica gel to give a filtrate. The resultant filtrate was concentrated and purified through silica gel column chromatography. At this time, a mixed solvent of chloroform/hexane=3/1 was used as the developing solvent. The resultant fraction was concentrated to give a solid, which was recrystallized with a mixed solvent of chloroform and methanol to give a powdery white solid of the intended product (compound 3) at a production quantity of 1.4 g and a yield of 97%.

¹H NMR (500 Hz, CDCl₃, δ): 8.91 (d, J=6.7 Hz, 2H), 8.90 (s, 2H), 8.71 (d, J=8.5 Hz, 2H), 7.94-7.92 (m, 2H), 7.84-7.80 (m, 2H), 7.73-7.70 (m, 4H), 7.58-7.49 (m, 5H), 7.73-7.30 (m, 4H), 7.20 (d, J=8.3 Hz, 2H), 7.06-7.01 (m, 2H)

MS: 673.61

### (Synthesis Example 6) Synthesis of Compound 9

### (6-1) Synthesis of Intermediate A-2

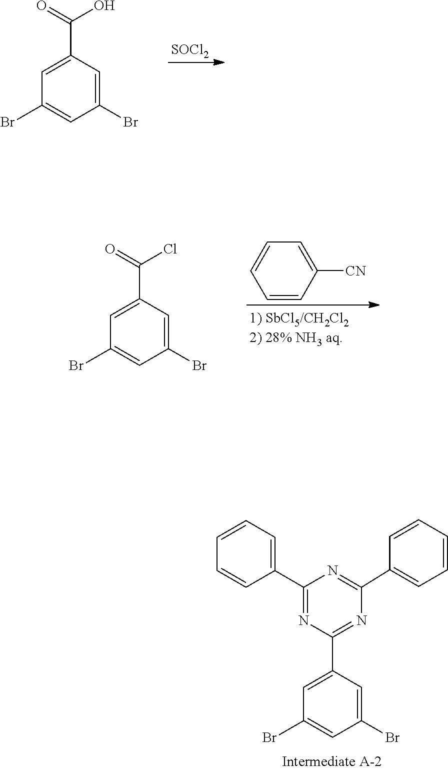 [SMILES: O=C(O)c1cc(Br)cc(Br)c1 ; "SOCl2" ; O=C(Cl)c1cc(Br)cc(Br)c1 ; N#Cc1ccccc1 ; Brc1cc(Br)cc(-c2nc(-c3ccccc3)nc(-c3ccccc3)n2)c1]

30 g (0.11 mol) of 3,5-dibromobenzoic acid was put into a 1000-mL three-neck flask, which was then purged with nitrogen, 24 mL of thionyl chloride and 3 drops of dimethylformamide were added thereto, and stirred in a nitrogen stream atmosphere at 70° C. for 3 hours. After stirring, thionyl chloride in the solution was removed through evaporation under reduced pressure, and the residue was dried for 3 hours. After drying, 22 g (0.21 mol) of benzonitrile was added, and stirred in a nitrogen stream atmosphere at 0° C. After stirring, 14 mL (0.11 mol) of antimony chloride was added, then gradually restored from 0° C. to room temperature and stirred at 60° C. for 1 hour. After stirring, the mixture was cooled, then 200 mL of aqueous ammonia was added and stirred at 0° C. The mixture was filtered under suction to give a solid. The resultant solid was washed with water and methanol in that order. After washing, the solid was transferred into an eggplant flask, then 200 mL of N,N-dimethylformamide was added thereto and stirred at 153° C. After stirring, the mixture was filtered under suction. The filtered residue was again transferred into an eggplant flask, then 100 mL of N,N-dimethylformamide was added thereto and stirred at 153° C. After stirring, the mixture was again filtered under suction. The resultant filtrate and the precipitated solid from the filtrate were put into an eggplant flask, and evaporated under reduced pressure to reduce N,N-dimethylformamide to be about 100 mL. 500 mL of water was added to the mixture, then stirred and filtered. The resultant solid was washed with water. The solid was added to 500 mL of methanol, irradiated with ultrasonic waves, and filtered under suction to give a white powder solid of the intended product (intermediate A-2: 2-(3,5-dibromophenyl)-4,6-diphenyl-1,3,5-triazine) at a production quantity of 22 g and a yield of 45%.

¹H NMR (500 Hz, CDCl₃, δ): 8.83 (d, J=2.4 Hz, 2H), 8.79-8.75 (m, 4H), 7.90 (t, J=2.0 Hz, 1H), 7.66-7.58 (m, 6H)

MS: 468.24

### (6-2) Synthesis of Compound 9

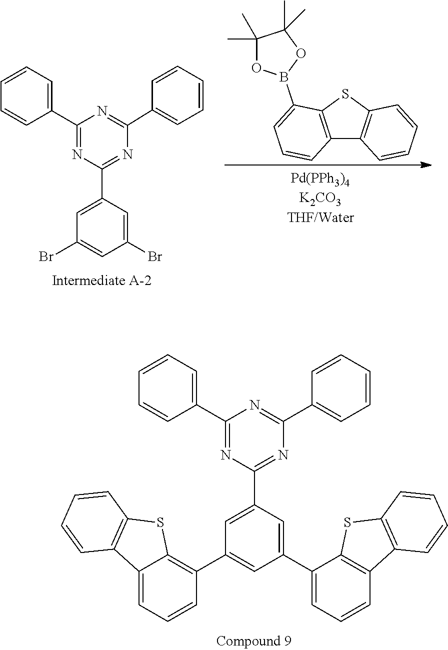 [SMILES: Brc1cc(Br)cc(-c2nc(-c3ccccc3)nc(-c3ccccc3)n2)c1 ; CC1(C)OB(c2cccc3c2sc2ccccc23)OC1(C)C ; c1ccc(-c2nc(-c3ccccc3)nc(-c3cc(-c4cccc5c4sc4ccccc45)cc(-c4cccc5c4sc4ccccc45)c3)n2)cc1]

1.1 g (2.4 mmol) of the intermediate A2 (2-(3,5-dibromophenyl)-4,6-diphenyl-1,3,5-triazine), 1.8 g (5.8 mmol) of 2-(dibenzo[b,d]thiophen-4-yl)-4,4,5,5-tetramethyl-1,3,2-dioxaborolane, 0.080 g (0.069 mmol) of tetrakis(triphenylphosphine)palladium(0), and 11 g (80 mmol) of potassium carbonate were put into a 200-mL three-neck flask, which was then purged with nitrogen. 120 mL of tetrahydrofuran and 40 mL of water were added to the mixture, and stirred in a nitrogen atmosphere at 95° C. for 24 hours. After stirring, the mixture was filtered under suction to give a solid. The resultant solid was washed with water and acetone in that order to give a powdery white solid of the intended product (compound 9) at a production quantity of 1.3 g and a yield of 82%.

¹H NMR (500 Hz, CDCl₃, δ): 9.27 (s, 2H), 8.82 (dd, J=8.2 Hz, 1.5 Hz, 4H), 8.36 (t, J=1.8 Hz, 1H), 8.27-8.24 (m, 4H), 7.89-7.87 (m, 2H), 7.75 (dd, J=7.7 Hz, 1.2 Hz, 2H), 7.68 (t, J=7.5 Hz, 2H), 7.62-7.54 (m, 6H), 7.53-7.26 (m, 4H)

MS: 673.47

### (Synthesis Example 7) Synthesis of Compound 10

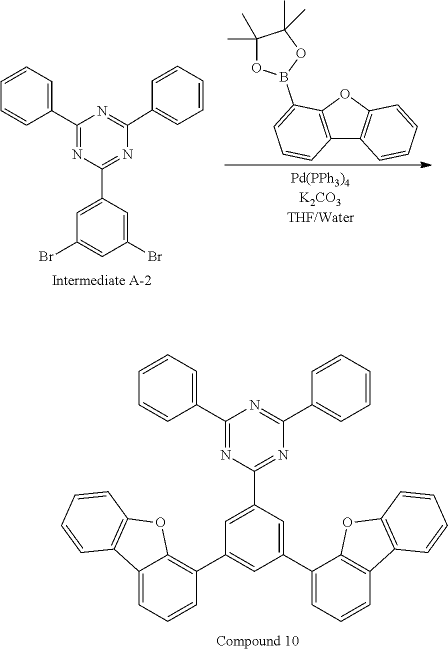 [SMILES: Brc1cc(Br)cc(-c2nc(-c3ccccc3)nc(-c3ccccc3)n2)c1 ; CC1(C)OB(c2cccc3c2oc2ccccc23)OC1(C)C ; c1ccc(-c2nc(-c3ccccc3)nc(-c3cc(-c4cccc5c4oc4ccccc45)cc(-c4cccc5c4oc4ccccc45)c3)n2)cc1]

1.5 g (3.1 mmol) of the intermediate A-2 (2-(3,5-dibromophenyl)-4,6-diphenyl-1,3,5-triazine) synthesized in the same manner as in Synthesis Example 6, 2.2 g (7.5 mmol) of 2-(dibenzo[b,d]furan-4-yl)-4,4,5,5-tetramethyl-1,3,2-dioxaborolane, 0.080 g (0.069 mmol) of tetrakis(triphenylphosphine)palladium(0), and 11 g (80 mmol) of potassium carbonate were put into a 200-mL three-neck flask, which was then purged with nitrogen. 120 mL of tetrahydrofuran and 40 mL of water were added to the mixture, and stirred in a nitrogen atmosphere at 95° C. for 24 hours. After stirring, the mixture was filtered under suction to give a solid. The resultant solid was washed with water and acetone in that order to give a powdery white solid of the intended product (compound 10) at a production quantity of 1.5 g and a yield of 75%.

¹H NMR (500 Hz, CDCl₃, δ): 9.42 (d, J=1.7 Hz, 2H), 8.86 (dd, J=8.0 Hz, 1.5 Hz, 4H), 8.72 (s, 1H), 8.07-8.05 (m, 4H), 7.98 (d, J=7.8 Hz, 2H), 7.67 (d, J=8.2 Hz, 2H), 7.63-7.55 (m, 8H), 7.51 (td, J=7.7 Hz, 1.3 Hz, 2H), 7.41 (td, J=7.7 Hz, 1.5 Hz, 2H)

MS: 642.61

### (Synthesis Example 8) Synthesis of Compound 11

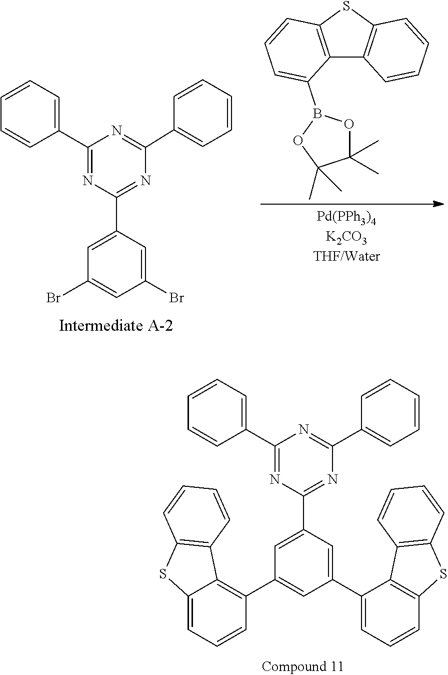 [SMILES: Brc1cc(Br)cc(-c2nc(-c3ccccc3)nc(-c3ccccc3)n2)c1 ; CC1(C)OB(c2cccc3sc4ccccc4c23)OC1(C)C ; c1ccc(-c2nc(-c3ccccc3)nc(-c3cc(-c4cccc5sc6ccccc6c45)cc(-c4cccc5sc6ccccc6c45)c3)n2)cc1]

1.0 g (2.1 mmol) of the intermediate A-2 (2-(3,5-dibromophenyl)-4,6-diphenyl-1,3,5-triazine) synthesized in the same manner as in Synthesis Example 6, 1.6 g (5.2 mmol) of 2-(dibenzo[b,d]thiophen-4-yl)-4,4,5,5-tetramethyl-1,3,2-dioxaborolane, 0.070 g (0.061 mmol) of tetrakis(triphenylphosphine)palladium(0), and 5.5 g (40 mmol) of potassium carbonate were put into a 200-mL three-neck flask, which was then purged with nitrogen. 60 mL of tetrahydrofuran and 20 mL of water were added to the mixture, and stirred in a nitrogen atmosphere at 95° C. for 24 hours. After stirring, the mixture was added to 100 mL of chloroform, and washed with water added thereto. After washing, the organic layer and the aqueous layer were separated, and the organic layer was filtered under suction through Celite and silica gel to give a filtrate. The resultant filtrate was concentrated and purified through silica gel chromatography. At this time, a mixed solvent of chloroform/hexane=3/1 was used as the developing solvent. The resultant fraction was concentrated, and the resultant solid was recrystallized in a mixed solvent of chloroform and methanol to give a powdery white solid of the intended product (compound 11) at a production quantity of 1.3 g and a yield of 90%.

¹H NMR (500 Hz, CDCl₃, δ): 9.06 (dd, J=5.8 Hz, 1.7 Hz, 2H), 8.72 (dd, J=8.3 Hz, 1.2 Hz, 4H), 7.94 (d, J=7.0 Hz, 4H), 7.93-7.86 (m, 3H), 7.84 (d, J=7.2 Hz, 1H), 7.80-7.49 (m, 9H), 7.44 (d, J=6.3 Hz, 2H), 7.37 (td, J=8.1 Hz, 1.0 Hz, 1H), 7.33 (td, J=8.1 Hz, 1.0 Hz, 1H), 7.17 (td, J=8.3 Hz, 1.0 Hz, 1H), 7.03 (td, J=8.3 Hz, 1.0 Hz, 1H)

MS: 674.62

### (Synthesis Example 9) Synthesis of Compound 4

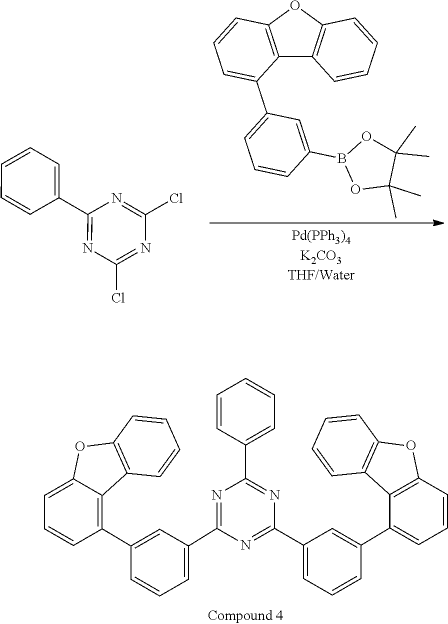 [SMILES: Clc1nc(Cl)nc(-c2ccccc2)n1 ; CC1(C)OB(c2cccc(-c3cccc4oc5ccccc5c34)c2)OC1(C)C ; c1ccc(-c2nc(-c3cccc(-c4cccc5oc6ccccc6c45)c3)nc(-c3cccc(-c4cccc5oc6ccccc6c45)c3)n2)cc1]

1.45 g (3.1 mmol) of 2,4-dichloro-6-phenyl-1,3,5-triazine, 2.75 g (7.44 mmol) of 2-(3-dibenzo[b,d]furan-1-yl)phenyl)-4,4,5,5-tetramethyl-1,3,2-dioxaborolane, 0.10 g (0.093 mmol) of tetrakis(triphenylphosphine)palladium(0), and 8.3 g (60 mmol) of potassium carbonate were put into a 200-mL three-neck flask, which was then purged with nitrogen. 90 mL of tetrahydrofuran and 30 mL of water were added to the mixture, and stirred in a nitrogen atmosphere at 90° C. for 20 hours. After stirring, a solid precipitated. The precipitated solid was recrystallized with 1,2-dichlorobenzene to give a powdery white solid of the intended product (compound 4) at a production quantity of 1.4 g and a yield of 70%.

¹H NMR (500 Hz, CDCl₃, δ): 9.05 (t, J=0.9 Hz, 2H), 8.85-8.87 (m, 2H), 8.73 (t, J=7.7 Hz, 2H), 7.51-7.58 (m, 11H), 7.35 (d, J=7.4 Hz, 2H), 7.56-7.62 (m, 2H), 7.02 (t, J=8.0 Hz, 2H)

MS: 641.66

### (Synthesis Example 10) Synthesis of Compound 12

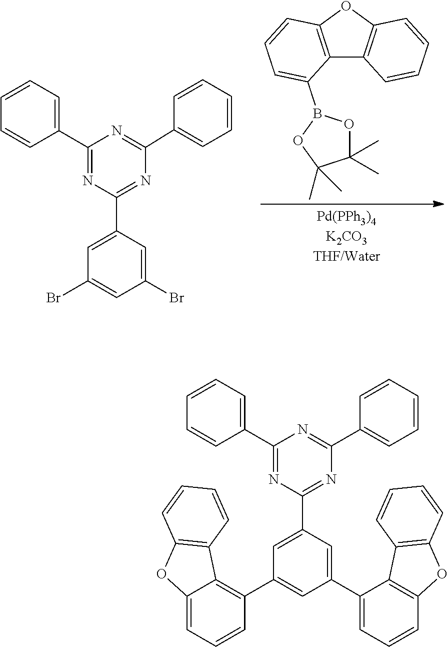 [SMILES: Brc1cc(Br)cc(-c2nc(-c3ccccc3)nc(-c3ccccc3)n2)c1 ; CC1(C)OB(c2cccc3oc4ccccc4c23)OC1(C)C ; c1ccc(-c2nc(-c3ccccc3)nc(-c3cc(-c4cccc5oc6ccccc6c45)cc(-c4cccc5oc6ccccc6c45)c3)n2)cc1]

1.45 g (3.1 mmol) of the intermediate A-2 (2-(3,5-dibromophenyl)-4,6-diphenyl-1,3,5-triazine) synthesized in the same manner as in Synthesis Example 6, 2.2 g (7.44 mmol) of 2-(dibenzo[b,d]furan-1-yl)-4,4,5,5-tetramethyl-1,3,2-dioxaborolane, 0.10 g (0.093 mmol) of tetrakis(triphenylphosphine)palladium(0), and 8.3 g (60 mmol) of potassium carbonate were put into a 200-mL three-neck flask, which was then purged with nitrogen. 90 mL of tetrahydrofuran and 30 mL of water were added to the mixture, and stirred in a nitrogen atmosphere at 90° C. for 20 hours. After stirring, the mixture was added to 100 mL of chloroform, and washed with water added thereto. After washing, a solid precipitated. The precipitated solid was recrystallized with 1,2-dichlorobenzene to give a powdery white solid of the intended product (compound 12) at a production quantity of 1.66 g and a yield of 83%.

¹H NMR (500 Hz, CDCl₃, δ): 9.18 (t, J=1.8 Hz, 2H), 8.73 (dd, J=7.2 Hz, 1.2 Hz, 2H), 8.12 (t, J=1.7 Hz, 1H), 7.81 (dd, J=7.9 Hz, 0.6 Hz, 2H), 7.66 (dd, J=7.3 Hz, 1.0 Hz, 2H), 7.62 (d, J=8.2 Hz, 2H), 7.56-7.60 (m, 4H), 7.48-7.52 (m, 6H), 7.42-7.45 (m, 2H), 7.12 (t, J=7.3 Hz, 2H)

MS: 641.66

### (Synthesis Example 11) Synthesis of Compound 80

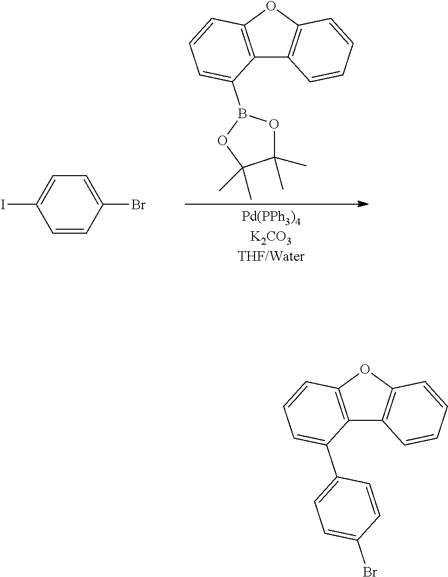 [SMILES: Brc1ccc(I)cc1 ; CC1(C)OB(c2cccc3oc4ccccc4c23)OC1(C)C ; Brc1ccc(-c2cccc3oc4ccccc4c23)cc1]

6.4 g (22.7 mmol) of 1-bromo-4-iodobenzene, 6.7 g (22.7 mmol) of 2-(dibenzo[b,d]furan-1-yl)-4,4,5,5-tetramethyl-1,3,2-dioxaborolane, 0.79 g (0.68 mmol) of tetrakis(triphenylphosphine)palladium(0), and 6.88 g (49.8 mmol) of potassium carbonate were put into a 200-mL three-neck flask, which was then purged with nitrogen. 50 mL of tetrahydrofuran and 25 mL of water were added to the mixture, and stirred in a nitrogen atmosphere at 80° C. for 12 hours. After stirring, the mixture was added to chloroform, and washed with water added thereto. After washing, the organic layer and the aqueous layer were separated, and the organic layer was filtered under suction through Celite and silica gel to give a filtrate. The resultant filtrate was concentrated and purified through silica gel column chromatography. At this time, a mixed solvent of chloroform/hexane=¼ was used as the developing solvent. The resultant fraction was concentrated to give a powdery white solid of the intended product (1-(4-bromophenyl)dibenzo[b,d]furan) at a production quantity of 5.2 g and a yield of 70.8 g.

¹H NMR (500 Hz, CDCl₃, δ): 7.67 (d, J=8.5 Hz, 2H), 7.56-7.59 (m, 2H), 7.48-7.51 (m, 4H), 7.41-7.44 (m, 1H), 7.21 (dd, J=7.5 Hz, 0.6 Hz, 1H), 7.13-7.17 (m, 1H)

MS: 323.08

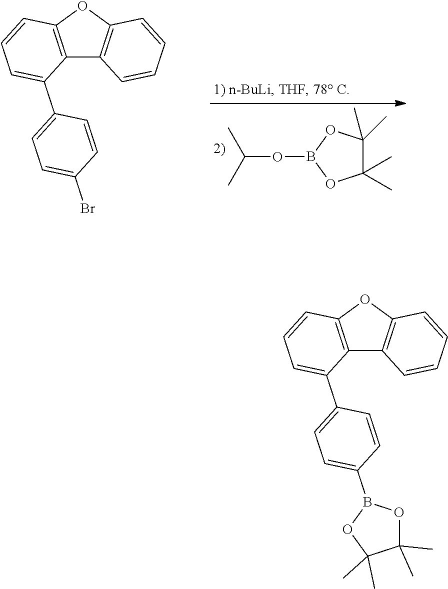 [SMILES: Brc1ccc(-c2cccc3oc4ccccc4c23)cc1 ; CC(C)OB1OC(C)(C)C(C)(C)O1 ; CC1(C)OB(c2ccc(-c3cccc4oc5ccccc5c34)cc2)OC1(C)C]

5.0 g (15.47 mmol) of (1-(4-bromophenyl)dibenzo[b,d]furan) was put into a 300-mL three-neck flask, which was then purged with nitrogen, and 80 mL of tetrahydrofuran was added thereto and stirred in a nitrogen atmosphere at −78° C. for 1 hour. To the solution, added was 10.2 mL (16.24 mmol of 1.6 mol/L n-butyllithium/hexane solution, and the solution was stirred at −78° C. for 1 hour. After stirring, 3.17 g (17.00 mmol) of 2-isopropoxy-4,4,5,5-tetramethyl-1,3,2-dioxaborolane was added to the solution, and gradually restored from −78° C. to room temperature, and stirred at room temperature for 12 hours. After stirring, 100 mL of water and 100 mL of chloroform were added to the solution and stirred. After stirring, the aqueous layer and the organic layer were separated, and the organic layer was washed with saturated saline water. After washing, the organic layer was dried with magnesium sulfate added thereto. After drying, the mixture was filtered under suction to give a filtrate. The resultant filtrate was concentrated and purified through silica gel column chromatography. At this time, a mixed solvent of chloroform/hexane=½ was used as the developing solvent. The resultant fraction was concentrated to give a transparent liquid of the intended product (2-[4-(dibenzo[b,d]furan-1-yl)phenyl]-4,4,5,5-tetramethyl-1,3,2-dioxaborolane at a production quantity of 3.4 g and a yield of 59.6%.

¹H NMR (500 Hz, CDCl₃, δ): 7.98 (d, J=7.9 Hz, 2H), 7.65 (d, J=7.9 Hz, 2H), 7.54-7.58 (m, 3H), 7.49 (t, J=7.6 Hz, 1H), 7.25 (dd, J=7.0 Hz, 0.7 Hz, 1H), 7.12 (t, J=7.5 Hz, 1H), 1.41 (s, 12H)

MS: 370.34

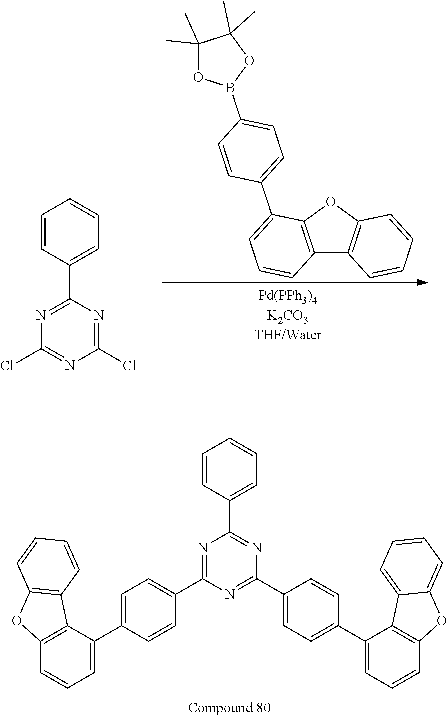 [SMILES: Clc1nc(Cl)nc(-c2ccccc2)n1 ; CC1(C)OB(c2ccc(-c3cccc4c3oc3ccccc34)cc2)OC1(C)C ; c1ccc(-c2nc(-c3ccc(-c4cccc5oc6ccccc6c45)cc3)nc(-c3ccc(-c4cccc5oc6ccccc6c45)cc3)n2)cc1]

0.70 g (3.1 mmol) of 2,4-dichloro-6-phenyl-1,3,5-triazine, 2.8 g (7.4 mmol) of (2-[4-(dibenzo[b,d]furan-1-yl)phenyl]-4,4,5,5-tetramethyl-1,3,2-dioxaborolane, 0.10 g (0.087 mmol) of tetrakis(triphenylphosphine)palladium(0), and 5.5 g (40 mmol) of potassium carbonate were put into a 200-mL three-neck flask, which was then purged with nitrogen. 90 mL of tetrahydrofuran and 30 mL of water were added to the mixture, and stirred in a nitrogen atmosphere at 95° C. for 24 hours. After stirring, the mixture was filtered under suction to give a solid. The resultant solid was washed with water and acetone in that order to give a powdery white solid of the intended product (compound 80) at a production quantity of 1.31 g and a yield of 65.5%.

¹H NMR (500 Hz, CDCl₃, δ): 9.01 (d, J=8.5 Hz, 4H), 8.89 (dd, J=7.5 Hz, 1.6 Hz, 2H), 7.90 (d, J=8.5 Hz, 4H), 7.54-7.90 (m, 11H), 7.42-7.46 (m, 2H), 7.37 (d, J=7.5 Hz, 2H), 7.17 (t, J=8.0 Hz, 2H)

MS: 641.39

### [1] Production of Organic Electroluminescent Device Using Compound 1 as Host Material in Light Emitting Layer, and Evaluation of Light Emission Characteristics Thereof

### Example 1

On a glass substrate having, formed thereon, an anode of indium-tin oxide (ITO) having a thickness of 100 nm, thin films were layered according to a vacuum evaporation method at a vacuum degree of 1×10⁻⁶ Pa. First, HAT-CN was formed on ITO to have a thickness of 10 nm. Next, Tris-PCz was formed to have a thickness of 20 nm, and mCBP was formed thereon to have a thickness of 10 nm. Next, the compound 1 and 4CzIPN were co-evaporated from different evaporation sources to form a layer having a thickness of 30 nm to be a light emitting layer. At this time, the ratio by weight of the compound 1 to 4CzIPN (compound ¼CzIPN) was 85 wt. %/15 wt. %. Next, T2T and Liq were co-evaporated from different evaporation sources to form a layer having a thickness of 10 nm. At this time, the ratio by weight of T2T to Liq (T2T/Liq) was 50 wt. %/50 wt. %. Next, Bpy-Tp2 and Liq were co-evaporated from different evaporation sources to form a layer having a thickness of 40 nm. At this time, the ratio by weight of P Bpy-Tp2 to Liq (Bpy-Tp2/Liq) was 70 wt. %/30 wt. %. Further, a layer of Liq was formed to have a thickness of 1 nm, and aluminum (Al) was vapor-deposited thereon to have a thickness of 100 nm to be a cathode, thereby producing an organic electroluminescent device.

### Comparative Example 1

An organic electroluminescent device was produced in the same manner as in Example 1 except that the compound 1 was changed to mCBP to form a layer thereof.

The layer configurations of the organic electroluminescent devices produced in Example 1 and Comparative Example 1 are shown in Table 16.

A voltage was applied to the organic electroluminescent devices produced in those Examples under the controlled condition that the brightness of each device could be 1000 cd/m² or 3000 cd/m², to measure the light emission spectrum and the external quantum efficiency thereof, and the results are shown in Table 17.

TABLE 16

| | | Hole | Hole | Electron | | | | Electron | |
|---|---|---|---|---|---|---|---|---|---|
| | | Injection | Transport | Blocking | Light | Hole | Electron | Injection | |
| No. | Anode | Layer | Layer | Layer | Emitting Layer | Blocking Layer | Transport Layer | Layer | Cathode |
| Example 1 | ITO | HAT-CN | Tris-PCz | mCBP | Compound 1:4CzIPN | T2T:Liq | Bpy-Tp2:Liq | Liq | Al |
| | | (10 nm) | (20 nm) | (10 nm) | (85 wt %:15 wt %) | (50 wt %:50 wt %) | (70 wt %:30 wt %) | (1 nm) | (100 nm) |
| | | | | | (30 nm) | (10 nm) | (40 nm) | | |
| Com- | ITO | HAT-CN | Tris-PCz | mCBP | mCBP:4CzIPN | T2T:Liq | Bpy-Tp2:Liq | Liq | Al |
| parative | | (10 nm) | (20 nm) | (10 nm) | (85 wt %:15 wt %) | (50 wt %:50 wt %) | (70 wt %:30 wt %) | (1 nm) | (100 nm) |
| Example 1 | | | | | (30 nm) | (10 nm) | (40 nm) | | |

In Table 16, “/” indicates a boundary between layers, and means that the layer on the left side of “/” and the layer on the right side of “/” are layered. The numerical value with the parenthesized unit nm shows the thickness of each layer. The same shall apply to the following Tables 19 and 20.

TABLE 17

| | Bright ness 1000 cd/m² | | Brightness 3000 cd/m² | |
|---|---|---|---|---|
| | | External | | External |
| | Peak | Quantum | Peak | Quantum |
| | Wavelength | Efficiency | Wavelength | Efficiency |
| No. | (nm) | (%) | (nm) | (%) |
| Example 1 | 535.1 | 11.60 | 533.0 | 9.93 |
| Compar- | 528.6 | 9.51 | 528.5 | 7.65 |
| ative | | | | |
| Example 1 | | | | |

As shown in Table 17, it is known that, when the compound 1 is used as the host material in the light emitting layer, an organic electroluminescent device having a high external quantum efficiency can be realized.

### [2] Thermal Stability of Compounds 1 to 4 and 9 to 12, Production of Organic Electroluminescent Devices Using any of Compounds 1 to 4 and 9 to 12 as Hole Blocking Material and Evaluation of Thermal Stability Thereof

### Experimental Example 1

Each of the compounds 1 to 4 and 9 to 12 synthesized in Synthesis Examples was analyzed through differential scanning calorimetry to measure the glass transition temperature (Tg) thereof, and the results are shown in Table 18.

TABLE 18

| | Glass Transition Temperature |
|---|---|
| Compound No. | (° C.) |
| Compound 1 | 120.7 |
| Compound 2 | 102.3 |
| Compound 3 | 136.2 |
| Compound 4 | 114.0 |
| Compound 9 | 134.5 |
| Compound 10 | not detected |
| Compound 11 | 131.8 |
| Compound 12 | 121.3 |

As shown in Table 18, the compounds 1 to 4, 9, 11 and 12 all have a glass transition temperature (Tg) of higher than 100° C., and are confirmed to hardly undergo crystallization at a high temperature and to have high thermal stability.

### Example 2

On a glass substrate having, formed thereon, an anode of indium-tin oxide (ITO) having a thickness of 100 nm, thin films were layered according to a vacuum evaporation method at a vacuum degree of 1×10⁻⁶ Pa. First, HAT-CN was vapor-deposited on ITO to have a thickness of 10 nm to be a hole injection layer. Next, Tris-PCz was vapor-deposited to have a thickness of 20 nm to be a hole transport layer, and mCBP was vapor-deposited thereon to have a thickness of 10 nm to be an electron blocking layer. Next, mCBP and 4CzIPN were co-evaporated from different evaporation sources to form a layer having a thickness of 30 nm to be a light emitting layer. At this time, the ratio by weight of mCBP to 4CzIPN (mCBP/4CzIPN) was 85 wt. %/15 wt. %. Next, the compound 1 was vapor-deposited to have a thickness of 10 nm to be a hole blocking layer. Next, Bpy-Tp2 and Liq were co-evaporated from different evaporation sources to form a layer having a thickness of 40 nm to be an electron transport layer. At this time, the ratio by weight of P Bpy-Tp2 to Liq (Bpy-Tp2/Liq) was 70 wt. %/30 wt. %. Further, a layer of Liq was formed to have a thickness of 1 nm to be an electron injection layer, and aluminum (Al) was vapor-deposited thereon to have a thickness of 100 nm to be a cathode, thereby producing an organic electroluminescent device.

### Examples 3 to 9

Organic electroluminescent devices were produced in the same manner as in Example 2 except that the compound 1 was changed to the compound shown in the column of hole blocking layer in Table 19 to form the hole blocking layer.

### Comparative Example 2

An organic electroluminescent device was produced in the same manner as in Example 2 except that the compound 1 was changed to T2T to form the hole blocking layer.

The layer configurations of the organic electroluminescent devices produced in Examples 2 to 9 and Comparative Example 2 are shown in Table 19.

TABLE 19

| | | Hole | Hole | Electron | | Hole | | Electron | |
|---|---|---|---|---|---|---|---|---|---|
| | | Injection | Transport | Blocking | Light | Blocking | Electron | Injection | |
| No. | Anode | Layer | Layer | Layer | Emitting Layer | Layer | Transport Layer | Layer | Cathode |
| Example 2 | ITO | HAT-CN | Tris-PCz | mCBP | mCBP:4CzIPN | Compound 1 | Bpy-Tp2:Liq | Liq | Al |
| | | (10 nm) | (20 nm) | (10 nm) | (85 wt %:15 wt %) | (10 nm) | (70 wt %:30 wt %) | (1 nm) | (100 nm) |
| | | | | | (30 nm) | | (40 nm) | | |
| Example 3 | ITO | HAT-CN | Tris-PCz | mCBP | mCBP:4CzIPN | Compound 2 | Bpy-Tp2:Liq | Liq | Al |
| | | (10 nm) | (20 nm) | (10 nm) | (85 wt %:15 wt %) | (10 nm) | (70 wt %:30 wt %) | (1 nm) | (100 nm) |
| | | | | | (30 nm) | | (40 nm) | | |
| Example 4 | ITO | HAT-CN | Tris-PCz | mCBP | mCBP:4CzIPN | Compound 3 | Bpy-Tp2:Liq | Liq | Al |
| | | (10 nm) | (20 nm) | (10 nm) | (85 wt %:15 wt %) | (10 nm) | (70 wt %:30 wt %) | (1 nm) | (100 nm) |
| | | | | | (30 nm) | | (40 nm) | | |
| Example 5 | ITO | HAT-CN | Tris-PCz | mCBP | mCBP:4CzIPN | Compound 4 | Bpy-Tp2:Liq | Liq | Al |
| | | (10 nm) | (20 nm) | (10 nm) | (85 wt %:15 wt %) | (10 nm) | (70 wt %:30 wt %) | (1 nm) | (100 nm) |
| | | | | | (30 nm) | | (40 nm) | | |
| Example 6 | ITO | HAT-CN | Tris-PCz | mCBP | mCBP:4CzIPN | Compound 9 | Bpy-Tp2:Liq | Liq | Al |
| | | (10 nm) | (20 nm) | (10 nm) | (85 wt %:15 wt %) | (10 nm) | (70 wt %:30 wt %) | (1 nm) | (100 nm) |
| | | | | | (30 nm) | | (40 nm) | | |
| Example 7 | ITO | HAT-CN | Tris-PCz | mCBP | mCBP:4CzIPN | Compound 10 | Bpy-Tp2:Liq | Liq | Al |
| | | (10 nm) | (20 nm) | (10 nm) | (85 wt %:15 wt %) | (10 nm) | (70 wt %:30 wt %) | (1 nm) | (100 nm) |
| | | | | | (30 nm) | | (40 nm) | | |
| Example 8 | ITO | HAT-CN | Tris-PCz | mCBP | mCBP:4CzIPN | Compound 11 | Bpy-Tp2:Liq | Liq | Al |
| | | (10 nm) | (20 nm) | (10 nm) | (85 wt %:15 wt %) | (10 nm) | (70 wt %:30 wt %) | (1 nm) | (100 nm) |
| | | | | | (30 nm) | | (40 nm) | | |
| Example 9 | ITO | HAT-CN | Tris-PCz | mCBP | mCBP:4CzIPN | Compound 12 | Bpy-Tp2:Liq | Liq | Al |
| | | (10 nm) | (20 nm) | (10 nm) | (85 wt %:15 wt %) | (10 nm) | (70 wt %:30 wt %) | (1 nm) | (100 nm) |
| | | | | | (30 nm) | | (40 nm) | | |
| Comparative | ITO | HAT-CN | Tris-PCz | mCBP | mCBP:4CzIPN | T2T | Bpy-Tp2:Liq | Liq | Al |
| Example 2 | | (10 nm) | (20 nm) | (10 nm) | (85 wt %:15 wt %) | (10 nm) | (70 wt %:30 wt %) | (1 nm) | (100 nm) |
| | | | | | (30 nm) | | (40 nm) | | |

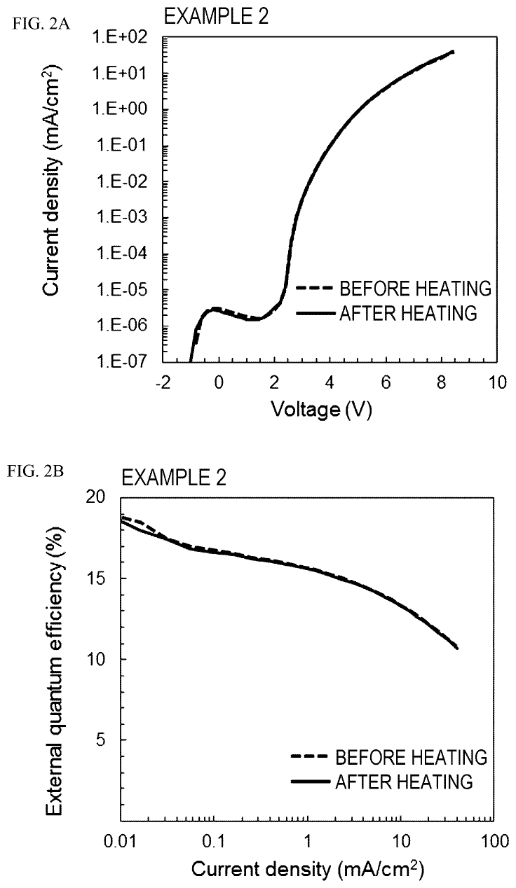

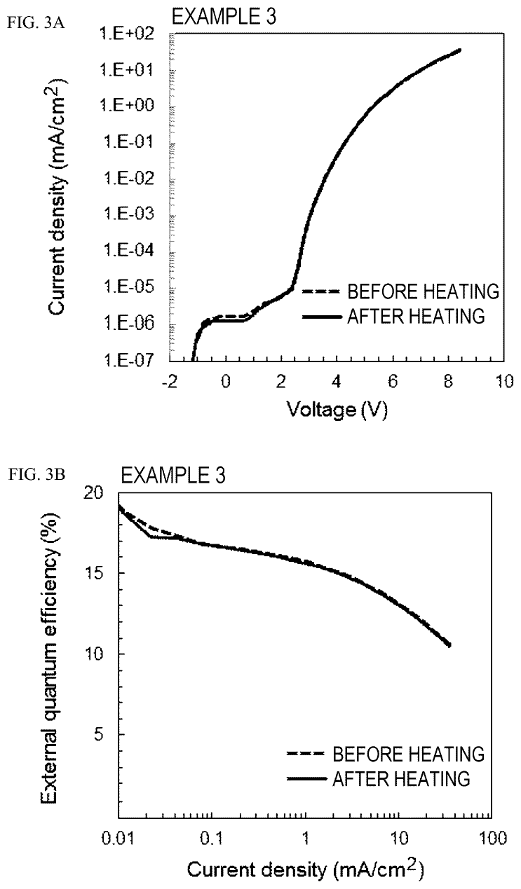

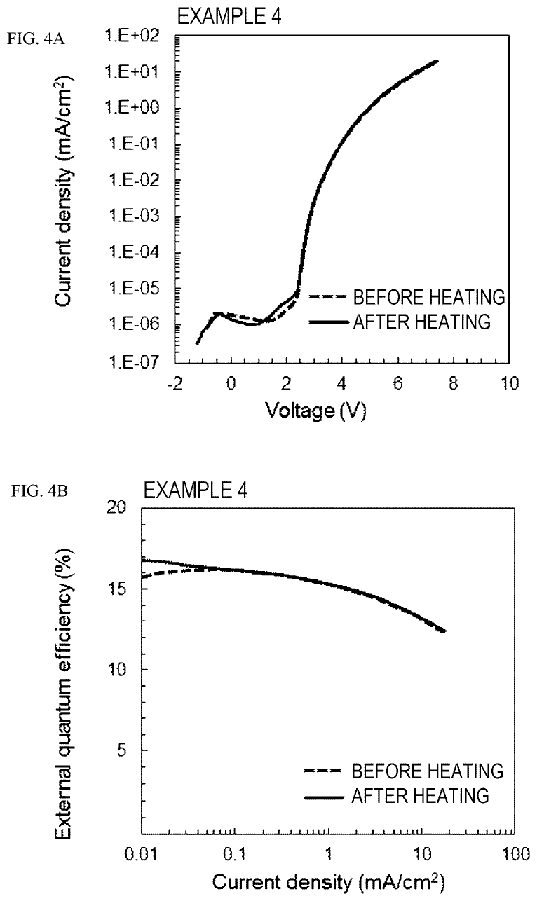

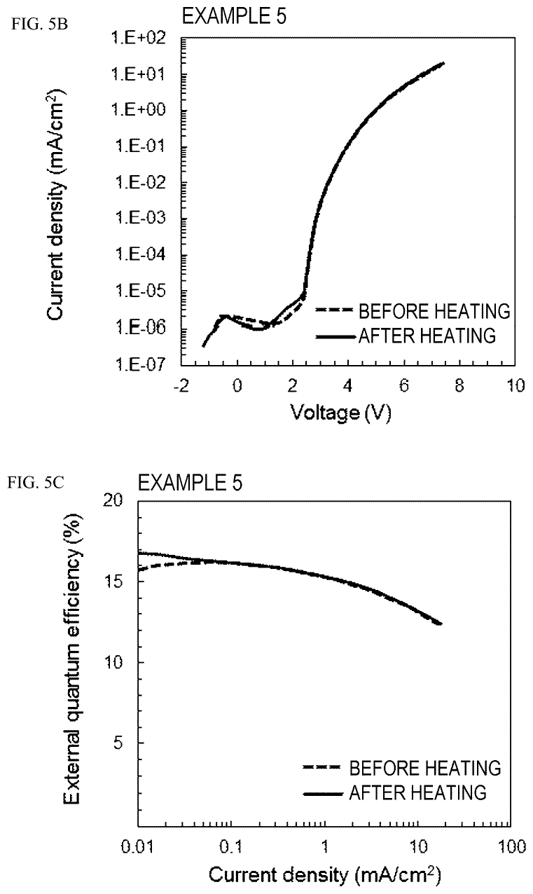

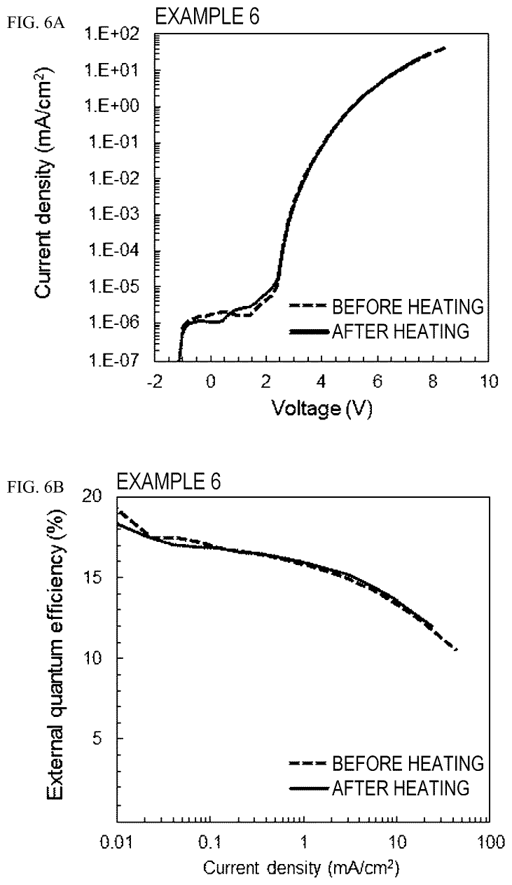

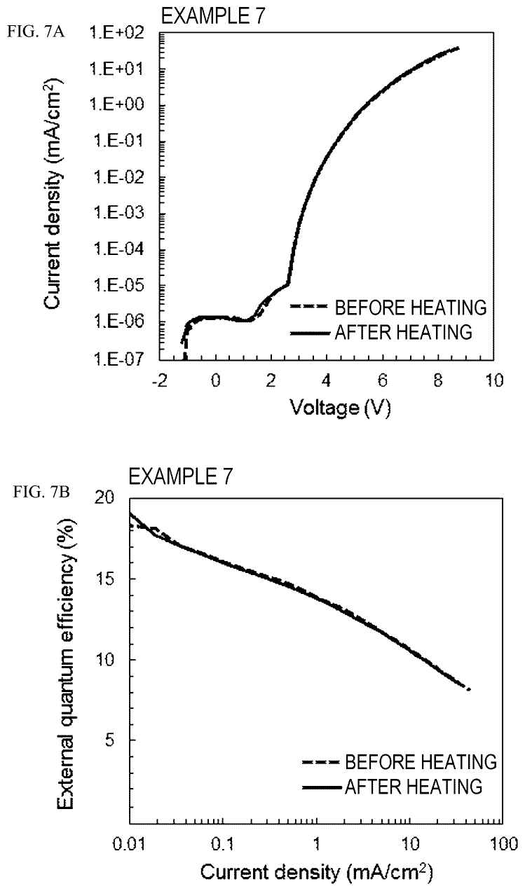

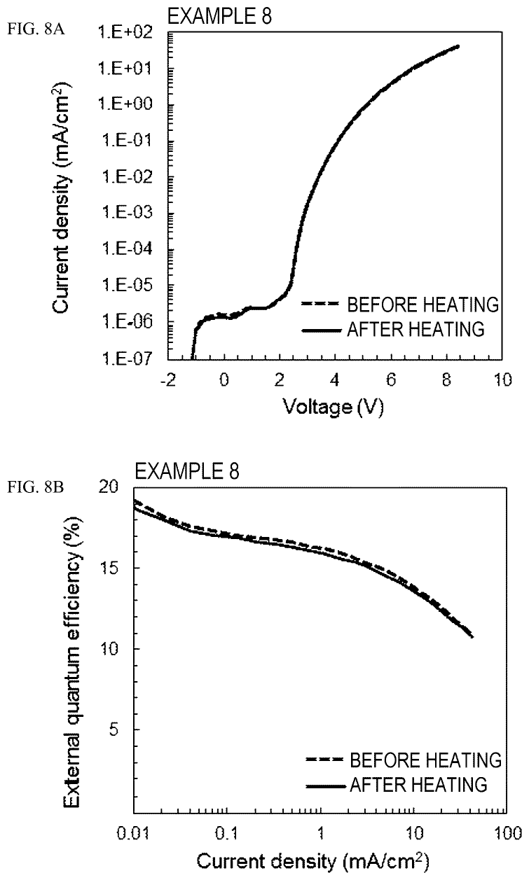

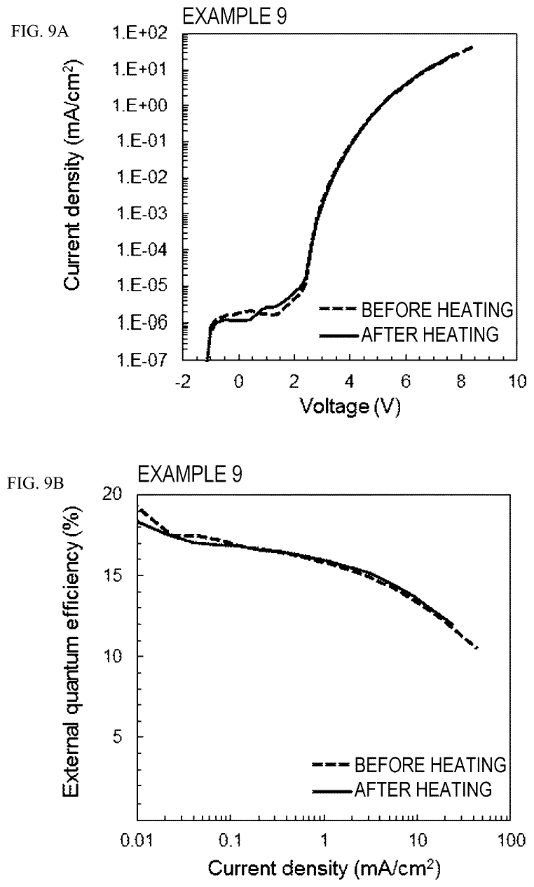

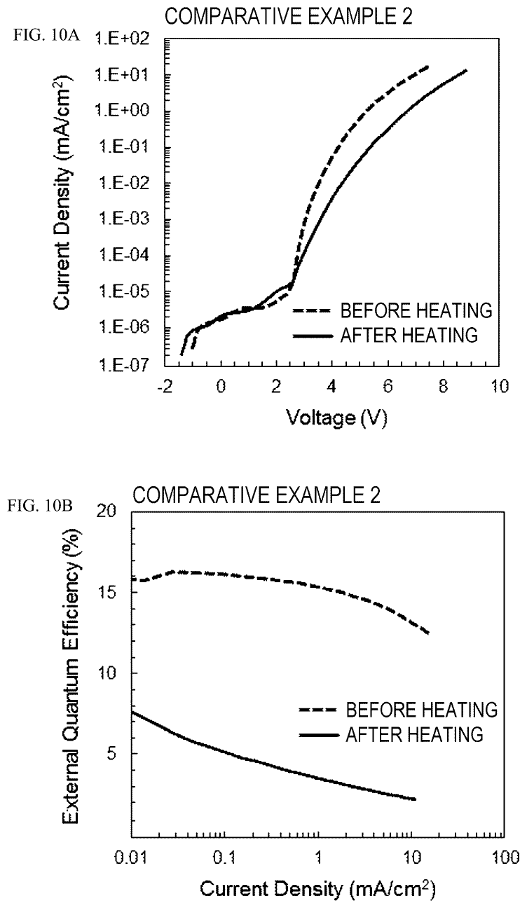

Before and after heated at 80° C. for 12 hours, the organic electroluminescent devices produced herein were driven to measure the voltage-current density characteristics and the current density-external quantum efficiency characteristics thereof. The results are shown in FIG. 2 to FIG. 10. In FIG. 2 to FIG. 10, FIGS. 2(a) and (b) each show the voltage-current density characteristics and the current density-external quantum efficiency characteristics of the organic electroluminescent device of Example 2; FIGS. 3(a) and (b) each show the voltage-current density characteristics and the current density-external quantum efficiency characteristics of the organic electroluminescent device of Example 3; FIGS. 4(a) and (b) each show the voltage-current density characteristics and the current density-external quantum efficiency characteristics of the organic electroluminescent device of Example 4; FIGS. 5B and 5C each show the voltage-current density characteristics and the current density-external quantum efficiency characteristics of the organic electroluminescent device of Example 5; FIGS. 6(a) and (b) each show the voltage-current density characteristics and the current density-external quantum efficiency characteristics of the organic electroluminescent device of Example 6; FIGS. 7(a) and (b) each show the voltage-current density characteristics and the current density-external quantum efficiency characteristics of the organic electroluminescent device of Example 7; FIGS. 8(a) and (b) each show the voltage-current density characteristics and the current density-external quantum efficiency characteristics of the organic electroluminescent device of Example 8; FIGS. 9(a) and (b) each show the voltage-current density characteristics and the current density-external quantum efficiency characteristics of the organic electroluminescent device of Example 9; FIGS. 10(a) and (b) each show the voltage-current density characteristics and the current density-external quantum efficiency characteristics of the organic electroluminescent device of Comparative Example 2.

From FIG. 10, it is recognized that the voltage-current density characteristics of the organic electroluminescent device of Comparative Example 2 using T2T worsened by heating, and the external quantum efficiency thereof tended to greatly lower. As opposed to this, FIG. 2 to FIG. 9 are referred to, in which the organic electroluminescent devices of Examples 2 to 9 using any of the compounds 1 to 4 and 9 to 12 of the present invention maintained their characteristics on the same level before and after heating, that is, the characteristics of the devices did not worsen by heating. From this, it is known that the compounds of the present invention are superior to T2T in point of enhancing the thermal stability of devices.

### [3] Production and Evaluation of Other Organic Electroluminescent Devices Using any of Compounds 1 to 4 and 9 to 12

### Examples 10 and 11

Organic electroluminescent devices were produced in the same manner as in Example 2 except that mCBP was changed to the compound 11 or 12 as described in the column of light emitting layer and 4CzIPN was changed to 4CzTPN to form the light emitting layer, and that the compound 1 was changed to T2T to form the hole blocking layer.

### Example 12

An organic electroluminescent device was produced in the same manner as in Example 2 except that the light emitting layer was formed by co-evaporation of mCBP, 4CzTPN and DBP in place of forming the light emitting layer by co-evaporation of mCBP and 4CzIPN and that the compound 1 was changed to the compound 11 to form the hole blocking layer. In forming the light emitting layer, the ratio by weight of mCBP, 4CzTPN and DBP (mCBP/4CzTPN/DBP) was 84 wt. %/15 wt. %/1 wt. %.

### Examples 13 and 14

Organic electroluminescent devices were produced in the same manner as in Example 12 except that mCBP was changed to the compound 11 or 12 described in the column of light emitting layer in Table 20 to form the light emitting layer, and that the compound 11 was changed to the compound described in the column of hole blocking layer in Table 20 to form the hole blocking layer.

### Example 15

An organic electroluminescent device was produced in the same manner as in Example 2 except that the compound 1 was changed to the compound 3 to form the hole blocking layer and that Bpy-Tp2 was changed to the compound 3 to form the electron transport layer.

### Example 16

An organic electroluminescent device was produced in the same manner as in Example 2 except that mCBP was changed to the compound 3 to form the light emitting layer, that the compound 1 was changed to the compound 3 to form the hole blocking layer and that Bpy-Tp2 was changed to the compound 3 to form the electron transport layer.

### Example 17

An organic electroluminescent device was produced in the same manner as in Example 2 except that the compound 1 was changed to the compound 4 to form the hole blocking layer and that Bpy-Tp2 was changed to the compound 4 to form the electron transport layer.

### Examples 18 to 20

Organic electroluminescent devices were produced in the same manner as in Example 2 except that mCBP was changed to the compound 4, 1 or 2 described in the column of light emitting layer in Table 20 to form the light emitting layer, that the compound 1 was changed to the compound described in the column of hole blocking layer in Table 20 to form the hole blocking layer, and that Bpy-Tp2 was changed to the compound 4, 1 or 2 described in the column of electron transport layer in Table 20 to form the electron transport layer.

The layer configurations of the organic electroluminescent devices produced in Examples 10 to 20 are shown in Table 20.

TABLE 20

| | | Hole | Hole | Electron | | Hole | | Electron | |
|---|---|---|---|---|---|---|---|---|---|
| | | Injection | Transport | Blocking | | Blocking | Electron | Injection | |
| No. | Anode | Layer | Layer | Layer | Light Emitting Layer | Layer | Transport Layer | Layer | Cathode |
| Example 10 | ITO | HAT-CN | Tris-PCz | mCBP | Compound 11:4CzTPN | T2T | Bpy-Tp2:Liq | Liq | Al |
| | | (10 nm) | (20 nm) | (10 nm) | (85 wt %:15 wt %) | (10 nm) | (70 wt %:30 wt %) | (1 nm) | (100 nm) |
| | | | | | (30 nm) | | (40 nm) | | |
| Example 11 | ITO | HAT-CN | Tris-PCz | mCBP | Compound 12:4CzTPN | T2T | Bpy-Tp2:Liq | Liq | Al |
| | | (10 nm) | (20 nm) | (10 nm) | (85 wt %:15 wt %) | (10 nm) | (70 wt %:30 wt %) | (1 nm) | (100 nm) |
| | | | | | (30 nm) | | (40 nm) | | |
| Example 12 | ITO | HAT-CN | Tris-PCz | mCBP | mCBP:4CzTPN:DBP | Com- | Bpy-Tp2:Liq | Liq | Al |
| | | (10 nm) | (20 nm) | (10 nm) | (84 wt %:15 wt %:1 wt %) | pound 11 | (70 wt %:30 wt %) | (1 nm) | (100 nm) |
| | | | | | (30 nm) | (10 nm) | (4 0nm) | | |
| Example 13 | ITO | HAT-CN | Tris-PCz | mCBP | Compound 11:4CzTPN:DBP | Com- | Bpy-Tp2:Liq | Liq | Al |
| | | (10 nm) | (20 nm) | (10 nm) | (84 wt %:15 wt %:1 wt %) | pound 11 | (70 wt %:30 wt %) | (1 nm) | (100 nm) |
| | | | | | (30 nm) | (10 nm) | (4 0 nm) | | |
| Example 14 | ITO | HAT-CN | Tris-PCz | mCBP | Compound 12:4CzTPN:DBP | Com- | Bpy-Tp2:Liq | Liq | Al |
| | | (10 nm) | (20 nm) | (10 nm) | (84 wt %:15 wt %:1 wt %) | pound 12 | (70 wt %:30 wt %) | (1 nm) | (100 nm) |
| | | | | | (30 nm) | (10 nm) | (40 nm) | | |
| Example 15 | ITO | HAT-CN | Tris-PCz | mCBP | mCBP:4CzIPN | Com- | Compound 3:Liq | Liq | Al |
| | | (10 nm) | (20 nm) | (10 nm) | (85 wt %:15 wt %) | pound 3 | (70 wt %:30 wt %) | (1 nm) | (100 nm) |
| | | | | | (30 nm) | (10 nm) | (40 nm) | | |
| Example 16 | ITO | HAT-CN | Tris-PCz | mCBP | Compound 3:4CzIPN | Com- | Compound 3:Liq | Liq | Al |
| | | (10 nm) | (20 nm) | (10 nm) | (85 wt %:15 wt %) | pound 3 | (70 wt %:30 wt %) | (1 nm) | (100 nm) |
| | | | | | (30 nm) | (10 nm) | (40 nm) | | |
| Example 17 | ITO | HAT-CN | Tris-PCz | mCBP | mCBP:4CzIPN | Com- | Compound 4:Liq | Liq | Al |
| | | (10 nm) | (20 nm) | (10 nm) | (85 wt %:15 wt %) | pound 4 | (70 wt %:30 wt %) | (1 nm) | (100 nm) |
| | | | | | (30 nm) | (10 nm) | (40 nm) | | |
| Example 18 | ITO | HAT-CN | Tris-PCz | mCBP | Compound 4:4CzIPN | Com- | Compound 4:Liq | Liq | Al |
| | | (10 nm) | (20 nm) | (10 nm) | (85 wt %:15 wt %) | pound 4 | (70 wt %:30 wt %) | (1 nm) | (100 nm) |
| | | | | | (30 nm) | (10 nm) | (40 nm) | | |
| Example 19 | ITO | HAT-CN | Tris-PCz | mCBP | Compound 1:4CzIPN | Com- | Compound 1:Liq | Liq | Al |
| | | (10 nm) | (20 nm) | (10 nm) | (85 wt %:15 wt %) | pound 1 | (70 wt %:30 wt %) | (1 nm) | (100 nm) |
| | | | | | (30 nm) | (10 nm) | (40 nm) | | |
| Example 20 | ITO | HAT-CN | Tris-PCz | mCBP | Compound 2:4CzIPN | Com- | Compound 2:Liq | Liq | Al |
| | | (10 nm) | (20 nm) | (10 nm) | (85 wt %:15 wt %) | pound 2 | (70 wt %:30 wt %) | (1 nm) | (100 nm) |
| | | | | | (30 nm) | (10 nm) | (40 nm) | | |

The organic electroluminescent devices produced in these Examples were driven to measure the external quantum efficiency thereof under the same condition as in Example 1 and to measure the thermal stability thereof under the same condition as in Example 2, which confirmed that the devices had high light emission efficiency and excellent thermal stability. In addition these organic electroluminescent devices were tested in a continuously driving test, and were confirmed to have high durability.

### [4] Evaluation of Light Emission Characteristics of Compound 80

A toluene solution of the compound 80 (10-5 mol/L) was prepared and measured for light emission spectrometry with 300 nm excitation light, which gave light emission at a peak wavelength of 392 nm. From the transient decay curves measured in the case with nitrogen bubbling and in the case without nitrogen bubbling, the fluorescence life (τ1) and the delayed fluorescence life (τ2) as shown in the following Table were determined. The results in the Table show that the compound of the present invention is useful as a delayed fluorescent material.

TABLE 21

| | τ1 | τ2 |
|---|---|---|
| with nitrogen bubbling | 0.45 ns | 4.5 ns |
| without nitrogen bubbling | 0.43 ns | 17.0 ns |

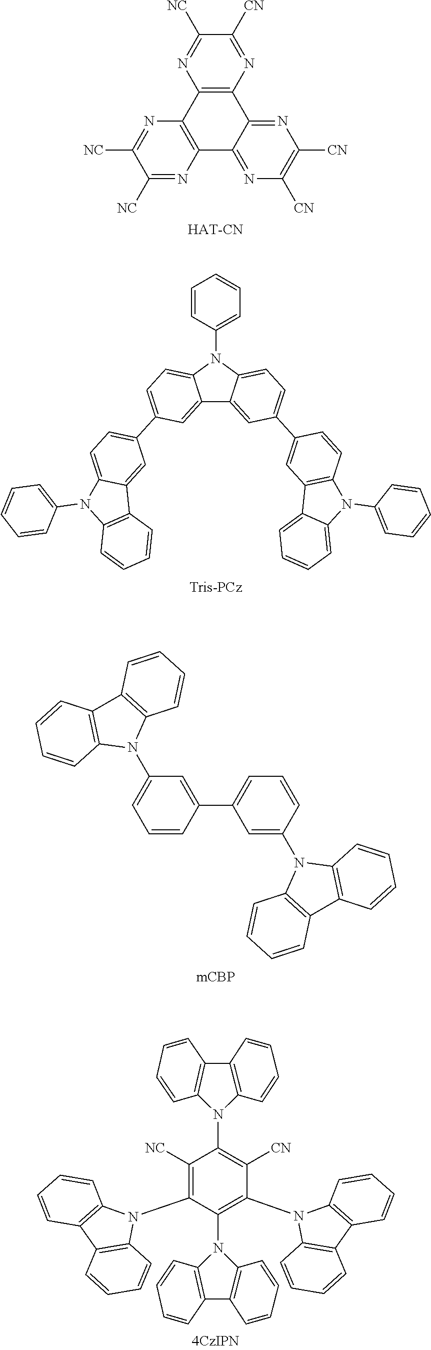 [SMILES: N#Cc1nc2c3nc(C#N)c(C#N)nc3c3nc(C#N)c(C#N)nc3c2nc1C#N ; c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; c1cc(-c2cccc(-n3c4ccccc4c4ccccc43)c2)cc(-n2c3ccccc3c3ccccc32)c1 ; N#Cc1c(-n2c3ccccc3c3ccccc32)c(C#N)c(-n2c3ccccc3c3ccccc32)c(-n2c3ccccc3c3ccccc32)c1-n1c2ccccc2c2ccccc21]

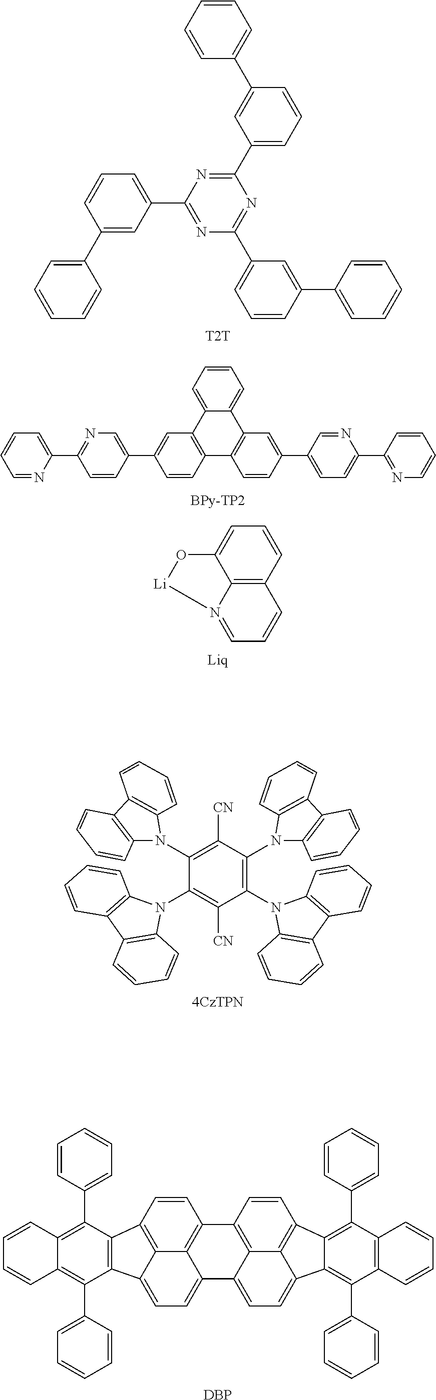

Organic electroluminescent devices were produced according to the same method as in Example 1 except that the compounds 1 to 300, and 302 to 1112 represented by the above-mentioned general formula (A) were used in place of 4CzIPN used in Example 1, and these devices are illustrated here as devices 1A to 300A, and 302A to 1112A.

Organic electroluminescent devices were produced according to the same method as in Example 1 except that the compounds 1 to 2785 represented by the above-mentioned general formula (B) were used in place of 4CzIPN used in Example 1, and these devices are illustrated here as devices 1B to 2785B.

Organic electroluminescent devices were produced according to the same method as in Example 1 except that the compounds 1 to 901 represented by the above-mentioned general formula (C) were used in place of 4CzIPN used in Example 1, and these devices are illustrated here as devices 1C to 901C.

Organic electroluminescent devices were produced according to the same method as in Example 1 except that the compounds 1 to 60084 represented by the above-mentioned general formula (D) were used in place of 4CzIPN used in Example 1, and these devices are illustrated here as devices 1D to 60084D.

Organic electroluminescent devices were produced according to the same method as in Example 1 except that the compounds 1 to 60 represented by the above-mentioned general formula (E) were used in place of 4CzIPN used in Example 1, and these devices are illustrated here as devices 1E to 60E.

Organic electroluminescent devices were produced according to the same method as in Example 1 except that four compounds represented by the above-mentioned general formula (F) were used in place of 4CzIPN used in Example 1, and these devices are illustrated here as devices 1F to 4F.

Organic electroluminescent devices were produced according to the same method as in Example 1 except that 11 compounds of the above-mentioned light emitting material group G were used in place of 4CzIPN used in Example 1, and these devices are illustrated here as devices 1G to 11G.

Organic electroluminescent devices produced according to the same method as in Example 1 except that, in place of HAT-CN used in Example 1, 8 compounds excepting HAT-CN described hereinabove as usable as a hole injection material were used are illustrated here as devices 1H to 8H.

Organic electroluminescent devices produced according to the same method as in Example 1 except that, in place of Tris-PCz used in Example 1, 36 compounds excepting Tris-PCz described hereinabove as usable as a hole transport material were used are illustrated here as devices 1I to 36I.

Organic electroluminescent devices produced according to the same method as in Example 1 except that, in place of mCBP used in Example 1, 8 compounds excepting mCBP described hereinabove as usable as an electron blocking material were used are illustrated here as devices 1J to 8J.

Organic electroluminescent devices produced according to the same method as in Example 1 except that, in place of T2T:Liq used in Example 1, 11 compounds described hereinabove as usable as a hole blocking material, and 34 compounds described hereinabove as usable as an electron transport material were used are illustrated here as devices 1K to 45K.

Organic electroluminescent devices produced according to the same method as in Example 1 except that, in place of Bpy-TP2:Liq used in Example 1, 3 compounds excepting LiF, CsF and Liq described hereinabove as usable as an electron injection material were used are illustrated here as devices 1L to 3L.

Organic electroluminescent devices produced according to the same method as in Example 1 except that the compounds 100001 to 102730 represented by the above-mentioned general formula (1) were used in place of the compound 1 used in Example 1 are illustrated here as devices 1M to 2730M.

Organic electroluminescent devices produced according to the same method as in Example 2 except that the compounds 1 to 300, and 302 to 1112 represented by the above-mentioned general formula (A) were used in place of 4CzIPN used in Example 2 are illustrated here as devices 1a to 300a, and 302a to 1112a.

Organic electroluminescent devices produced according to the same method as in Example 2 except that the compounds 1 to 2785 represented by the above-mentioned general formula (B) were used in place of 4CzIPN used in Example 2 are illustrated here as devices 1b to 2785b.

Organic electroluminescent devices produced according to the same method as in Example 2 except that the compounds 1 to 901 represented by the above-mentioned general formula (C) were used in place of 4CzIPN used in Example 2 are illustrated here as devices 1c to 901c.

Organic electroluminescent devices produced according to the same method as in Example 2 except that the compounds 1 to 60084 represented by the above-mentioned general formula (D) were used in place of 4CzIPN used in Example 2 are illustrated here as devices 1d to 60084d.

Organic electroluminescent devices produced according to the same method as in Example 2 except that the compounds 1 to 60 represented by the above-mentioned general formula (E) were used in place of 4CzIPN used in Example 2 are illustrated here as devices 1e to 60e.

Organic electroluminescent devices produced according to the same method as in Example 2 except that four compounds represented by the above-mentioned general formula (F) were used in place of 4CzIPN used in Example 2 are illustrated here as devices if to 4f.

Organic electroluminescent devices produced according to the same method as in Example 2 except that 11 compounds of the above-mentioned light emitting material group G were used in place of 4CzIPN used in Example 2 are illustrated here as devices 1g to 11g.

Organic electroluminescent devices produced according to the same method as in Example 2 except that, in place of HAT-CN used in Example 2, 8 compounds excepting HAT-CN described hereinabove as usable as a hole injection material were used are illustrated here as devices 1h to 8h.

Organic electroluminescent devices produced according to the same method as in Example 2 except that, in place of Tris-PCz used in Example 2, 36 compounds excepting Tris-PCz described hereinabove as usable as a hole transport material were used are illustrated here as devices 1i to 36i.

Organic electroluminescent devices produced according to the same method as in Example 2 except that, in place of mCBP used in Example 2, 8 compounds excepting mCBP described hereinabove as usable as an electron blocking material were used are illustrated here as devices 1j to 8j.

Organic electroluminescent devices produced according to the same method as in Example 2 except that, in place of T2T:Liq used in Example 2, 11 compounds described hereinabove as usable as a hole blocking material, and 34 compounds described hereinabove as usable as an electron transport material were used are illustrated here as devices 1k to 45k.

Organic electroluminescent devices produced according to the same method as in Example 2 except that, in place of Bpy-TP2:Liq used in Example 2, 3 compounds excepting LiF, CsF and Liq described hereinabove as usable as an electron injection material were used are illustrated here as devices 11 to 31.

Organic electroluminescent devices produced according to the same method as in Example 2 except that the compounds 100001 to 102730 represented by the above-mentioned general formula (1) were used in place of the compound 1 used in Example 2 are illustrated here as devices 1m to 2730m.

### INDUSTRIAL APPLICABILITY

The compound of the present invention is useful as a material for organic light emitting devices such as organic electroluminescent devices. For example, the compound is usable as a host material and an assist dopant for organic light emitting devices such as organic electroluminescent devices. Accordingly, the industrial applicability of the present invention is great.

### REFERENCE SIGNS LIST

1 Substrate
2 Anode
3 Hole Injection Layer
4 Hole Transport Layer
5 Light Emitting Layer
6 Electron Transport Layer
7 Cathode

LENGTHY TABLES
The patent application contains a lengthy table section. A copy of the table is available in electronic form from the USPTO web site (https://seqdata.uspto.gov/?pageRequest=docDetail&DocID=US20240260455A1). An electronic copy of the table will also be available from the USPTO upon request and payment of the fee set forth in 37 CFR 1.19(b)(3).

1. An organic light emitting device having a light emitting layer containing a delayed fluorescent material and a hole blocking layer containing a compound represented by the following general formula (1): 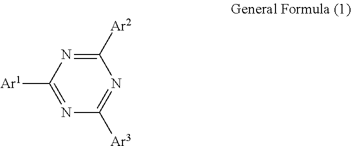
wherein Ar¹ to Ar³ each independently represent a substituted or unsubstituted aryl group or a substituted or unsubstituted heteroaryl group, and at least one of Ar¹ to Ar³ contains a skeleton represented by the following general formula (2), but Ar¹ to Ar³ do not contain a 4-(benzofuran-1-yl)carbazol-9-yl group or a 4-(benzothiophen-1-yl)carbazol-9-yl group, 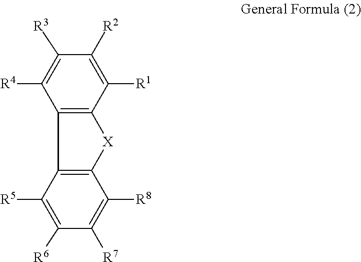
wherein X represents O or S, R¹ to R⁸ each independently represent a hydrogen atom, a substituent or a bonding position, R¹ and R², R² and R³, R³ and R⁴, R⁵ and R⁶, R⁶ and R⁷, and R⁷ and R⁸ each may bond to each other to form a cyclic structure.

2. The organic light emitting device according to claim 1, which is an organic light emitting device having a cathode, the light emitting layer, the hole blocking layer and an anode in this order.

3. The organic light emitting device according to claim 2, wherein the light emitting layer is in contact with the hole blocking layer.

4. The organic light emitting device according to claim 2, wherein an electron transport layer is provided between the hole blocking layer and the anode.

5. The organic light emitting device according to claim 1, wherein the hole blocking layer consists of a compound represented by the general formula (1).

6. The organic light emitting device according to claim 1, wherein the light emitting layer also contains a compound represented by the general formula (1).

7. The organic light emitting device according to claim 6, wherein the light emitting layer contains the compound represented by the general formula (1) in an amount of more than 50% by weight.

8. The organic light emitting device according to claim 6, wherein the light emitting layer consists of the delayed fluorescent material and the compound represented by the general formula (1).

9. The organic light emitting device according to claim 6, wherein the light emitting layer contains the delayed fluorescent material as an assist dopant, the compound represented by the general formula (1) and a light emitting material that emits delayed fluorescence.

10. The organic light emitting device according to claim 6, wherein the light emitting layer contains the delayed fluorescent material as an assist dopant, the compound represented by the general formula (1) and a light emitting material that does not emit delayed fluorescence.

11. The organic light emitting device according to claim 6, wherein the light emitting layer and the hole blocking layer contain the same compound represented by the general formula (1).

12. The organic light emitting device according to claim 1, wherein the delayed fluorescent material contained in the light emitting layer has a structure represented by the following general formula (A): 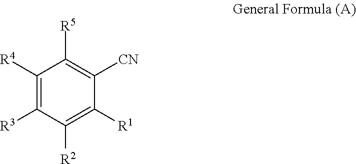
wherein at least one of R¹ to R⁵ represents a cyano group, at least one of R¹ to R⁵ represents a group represented by the following general formula (11), and the remaining R¹ to R⁵ each represent a hydrogen atom or a substituent, 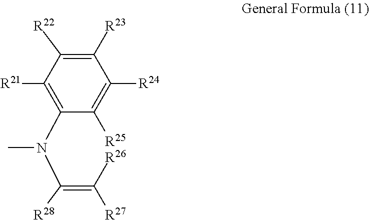
wherein R²¹ to R²⁸ each independently represent a hydrogen atom or a substituent, provided that at least one of the following <A> or <B> is satisfied:
<A> R²⁵ and R²⁶ together form a single bond, and
<B> R²⁷ and R²¹ together form an atomic group necessary for forming a substituted or unsubstituted benzene ring.

13. The organic light emitting device according to claim 1, wherein the delayed fluorescent material contained in the light emitting layer has a structure represented by the following general formula (B): 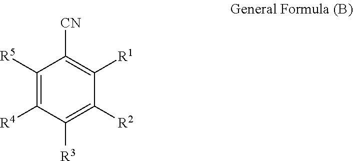
wherein one or more of R¹, R², R³, R⁴ and R⁵ each independently represent a 9-carbazolyl group having a substituent at at least one of 1-position and 8-position, a 10-phenoxazyl group having a substituent at at least one of 1-position and 9-position, or a 10-phenothiazyl group having a substituent at at least one of 1-position and 9-position; the remaining substituents each represent a hydrogen atom or a substituent, but the substituent is not a 9-carbazolyl group having a substituent at at least one of 1-position and 8-position, a 10-phenoxazyl group having a substituent at at least one of 1-position and 9-position, or a 10-phenothiazyl group having a substituent at at least one of 1-position and 9-position; and one or more carbon atoms constituting each ring skeleton of the 9-carbazolyl group, the 10-phenoxazyl group and the 10-phenothiazyl group may be substituted with a nitrogen atom.

14. The organic light emitting device according to claim 1, wherein the delayed fluorescent material contained in the light emitting layer has a structure represented by the following general formula (C): 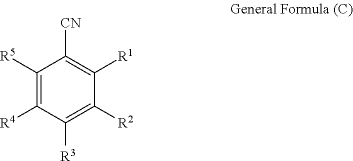
wherein three or more of R¹, R², R⁴ and R⁵ each independently represent a substituted or unsubstituted 9-carbazolyl group, a substituted or unsubstituted 10-phenoxazyl group, a substituted or unsubstituted 10-phenothiazyl group, or a cyano group; the remaining substituents each represent a hydrogen atom or a substituent, but the substituent is not a substituted or unsubstituted 9-carbazolyl group, a substituted or unsubstituted 10-phenoxazyl group, or a substituted or unsubstituted 10-phenothiazyl group; one or more carbon atoms constituting each ring skeleton of the 9-carbazolyl group, the 10-phenoxazyl group and the 10-phenothiazyl group may be substituted with a nitrogen atom; and R³ represents a hydrogen atom or a substituent, but the substituent is not a substituted or unsubstituted 9-carbazolyl group, a substituted or unsubstituted 10-phenoxazyl group, a cyano group, a substituted or unsubstituted 10-phenothiazyl group, a substituted or unsubstituted aryl group, a substituted or unsubstituted heteroaryl group or a substituted or unsubstituted alkynyl group.

15. The organic light emitting device according to claim 1, wherein the delayed fluorescent material contained in the light emitting layer has a structure represented by the following general formula (D): 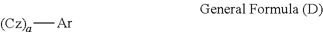
wherein Cz represents a 9-carbazolyl group having a substituent at at least one of 1-position and 8-position in which at least one carbon atom at the 1- to 8-positions constituting the ring skeleton of the carbazole ring of the 9-carbazolyl group may be substituted with a nitrogen atom, but both the 1-position and the 8-position are not substituted with a nitrogen atom, and each benzene ring constituting the 9-carbazolyl group may be condensed with any other ring,
Ar represents a benzene ring having a substituent containing a structural unit having a positive Hammett constant σₚ except a cyano group, or a biphenyl ring having a substituent containing a structural unit having a positive Hammett constant σₚ except a cyano group, and
a represents an integer of 1 or more, but is not more than the maximum number of the substituents with which the benzene ring or the biphenyl ring of Ar may be substituted, and when a is 2 or more, plural Cz's may be the same as or different from each other.

16. The organic light emitting device according to claim 1, wherein the delayed fluorescent material contained in the light emitting layer has a structure represented by the following general formula (E): 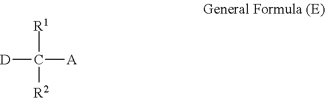
wherein R¹ and R² each independently represent a fluoroalkyl group, D represents a substituent having a negative Hammett constant σₚ, and A represents a substituent having a positive Hammett constant σₚ.

17. The organic light emitting device according to claim 1, wherein the delayed fluorescent material contained in the light emitting layer has a structure represented by the following general formula (F): 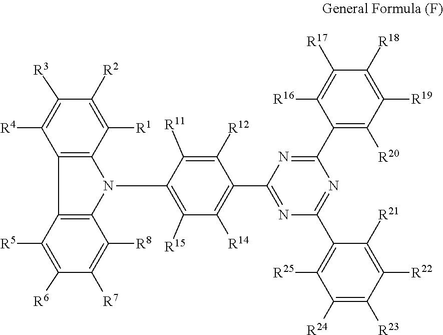
wherein R¹ to R⁸, R¹², and R¹⁴ to R²⁵ each independently represent a hydrogen atom or a substituent, and R¹¹ represents a substituted or unsubstituted alkyl group, provided that at least one of R² to R⁴ is a substituted or unsubstituted alkyl group, and at least one of R⁵ to R⁷ is a substituted or unsubstituted alkyl group.
